(19) United States
(12) Patent Application Publication
(10) Pub. No.: US 2025/0333399 A1
(43) Pub. Date: Oct. 30, 2025

(54) INHIBITORS OF FIBROBLAST GROWTH FACTOR RECEPTOR KINASES

(71) Applicant: Khora SPV 1, LLC, Boston, MA (US)

(72) Inventors: Stephen W. Kaldor, San Diego, CA (US); John Tyhonas, San Diego, CA (US); Eric A. Murphy, San Marcos, CA (US); Toufike Kanouni, Rancho Santa Fe, CA (US); Lee D. Arnold, Rancho Santa Fe, CA (US); Robert Kania, Del Mar, CA (US); Jason M. Cox, Rancho Santa Fe, CA (US)

(21) Appl. No.: 19/193,615

(22) Filed: Apr. 29, 2025

Related U.S. Application Data
(63) Continuation of application No. 17/702,444, filed on Mar. 23, 2022, now Pat. No. 12,331,039.
(60) Provisional application No. 63/035,155, filed on Jun. 5, 2020.
(60) Provisional application No. 63/106,812, filed on Oct. 28, 2020.

(51) Int. Cl. C07D 403/14; A61K 45/06; C07D 401/14; C07D 405/14; C07D 413/04; C07D 417/14; C07D 471/04; C07D 487/04

(52) U.S. Cl. CPC C07D 403/14; A61K 45/06; C07D 401/14; C07D 405/14; C07D 413/04; C07D 417/14; C07D 471/04; C07D 487/04

(57) ABSTRACT

Provided herein are heteroaryl inhibitors of fibroblast growth factor receptor kinases, pharmaceutical compositions comprising said compounds, and methods for using said compounds for the treatment of diseases.

### CROSS-REFERENCE TO RELATED APPLICATION

This is a continuation application of U.S. patent application Ser. No. 17/702,444, filed on Mar. 23, 2022, which is a continuation application of. U.S. patent application Ser. No. 17/339,123, filed on Jun. 4, 2021, and claims benefit of U.S. Patent Application No. 63/106,812, filed on Oct. 28, 2020, and U.S. Patent Application No. 63/035,155, filed on Jun. 5, 2020, all of which are hereby incorporated by reference in their entireties.

### BACKGROUND

Fibroblast growth factor receptors (FGFRs) are a subfamily of receptor tyrosine kinases (RTKs) that bind to members of the fibroblast growth factor family of proteins. Deregulation of the fibroblast growth factor/FGF receptor network occurs frequently in tumors. Accordingly, therapies that target abberant FGFR kinase activity are desired for use in the treatment of cancer and other disorders.

### BRIEF SUMMARY OF THE INVENTION

Provided herein are inhibitors of fibroblast growth factor receptor (FGFR) kinases, pharmaceutical compositions comprising said compounds, and methods for using said compounds for the treatment of diseases.

One embodiment provides a compound, or pharmaceutically acceptable salt or solvate thereof, having the structure of Formula (I):

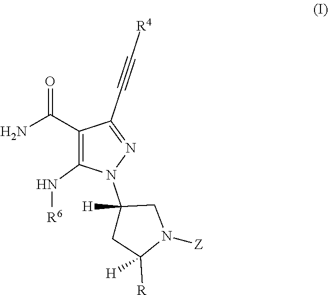

wherein,
Z is selected from a group having the structure:

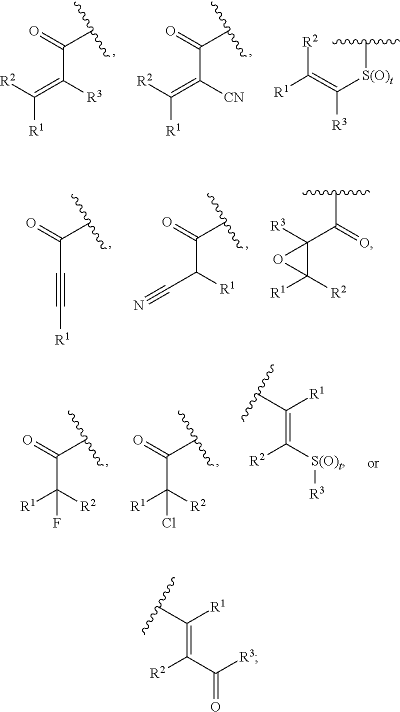

t is 1 or 2;
R¹, R², and R³ are each independently selected from hydrogen, fluoro, optionally substituted C1-C4 alkyl, or optional substituted heterocyclylalkyl;
R⁴ is an optionally substituted nitrogen-containing 9 or 10-atom heteroaryl;
R is selected from hydrogen, optionally substituted C1-C6 alkyl, optionally substituted C3-C7 carbocyclyl, optionally substituted C3-C7 carbocyclylalkyl, optionally substituted C3-C7 heterocyclyl, optionally substituted C3-C7 heterocyclylalkyl, optionally substituted C2-C7 alkenyl, —CO₂R⁵, —CONHR⁵, or —CON(R⁵)₂;
each R⁵ is independently selected from optionally substituted C1-C6 alkyl, optionally substituted C3-C7 carbocyclyl, optionally substituted C3-C7 carbocyclylalkyl, optionally substituted C3-C7 heterocyclyl, or optionally substituted C3-C7 heterocyclylalkyl; and
R⁶ is an optionally substituted alkyl, optionally substituted carbocyclylalkyl, or optionally substituted heterocyclylalkyl.

One embodiment provides a pharmaceutical composition comprising a compound of Formula (I), or pharmaceutically acceptable salt or solvate thereof, and at least one pharmaceutically acceptable excipient.

One embodiment provides a method of treating a disease or disorder in a patient in need thereof comprising administering to the patient a compound of Formula (I), or pharmaceutically acceptable salt or solvate thereof. Another embodiment provides the method wherein the disease or disorder is cancer.

### INCORPORATION BY REFERENCE

All publications, patents, and patent applications mentioned in this specification are herein incorporated by reference for the specific purposes identified herein.

### DETAILED DESCRIPTION OF THE INVENTION

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

### Definitions

As used in the specification and appended claims, unless specified to the contrary, the following terms have the meaning indicated below.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Hydrazino” refers to the ═N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C₂-C₈ alkenylene). In other embodiments, an alkenylene comprises two to five carbon atoms (e.g., C₂-C₅ alkenylene). In other embodiments, an alkenylene comprises two to four carbon atoms (e.g., C₂-C₄ alkenylene). In other embodiments, an alkenylene comprises two to three carbon atoms (e.g., C₂-C₃ alkenylene). In other embodiments, an alkenylene comprises two carbon atoms (e.g., C₂ alkenylene). In other embodiments, an alkenylene comprises five to eight carbon atoms (e.g., C₅-C₈alkenylene). In other embodiments, an alkenylene comprises three to five carbon atoms (e.g., C₃-C₅ alkenylene). Unless stated otherwise specifically in the specification, an alkenylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C₂-C₈ alkynylene). In other embodiments, an alkynylene comprises two to five carbon atoms (e.g., C₂-C₅ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atoms (e.g., C₂ alkynylene). In other embodiments, an alkynylene comprises five to eight carbon atoms (e.g., C₅-C₈alkynylene). In other embodiments, an alkynylene comprises three to five carbon atoms (e.g., C₃-C₅ alkynylene). Unless stated otherwise specifically in the specification, an alkynylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Aralkoxy” refers to a radical bonded through an oxygen atom of the formula —O—R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is saturated (i.e., containing single C—C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)OR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical is optionally substituted as defined above.

“Carbocyclylalkynyl” refers to a radical of the formula —R^{c}-carbocyclyl where R^{c} is an alkynylene chain as defined above. The alkynylene chain and the carbocyclyl radical is optionally substituted as defined above.

“Carbocyclylalkoxy” refers to a radical bonded through an oxygen atom of the formula —O—R^{c}-carbocyclyl where R^{c} is an alkylene chain as defined above. The alkylene chain and the carbocyclyl radical is optionally substituted as defined above.

As used herein, “carboxylic acid bioisostere” refers to a functional group or moiety that exhibits similar physical, biological and/or chemical properties as a carboxylic acid moiety. Examples of carboxylic acid bioisosteres include, but are not limited to,

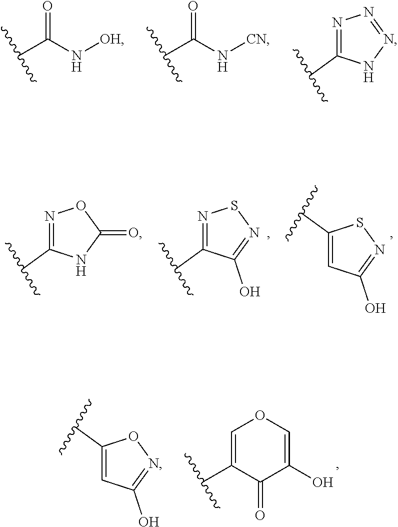

and the like.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heterocyclylalkyl” refers to a radical of the formula —R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O))ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)— or (S)—. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

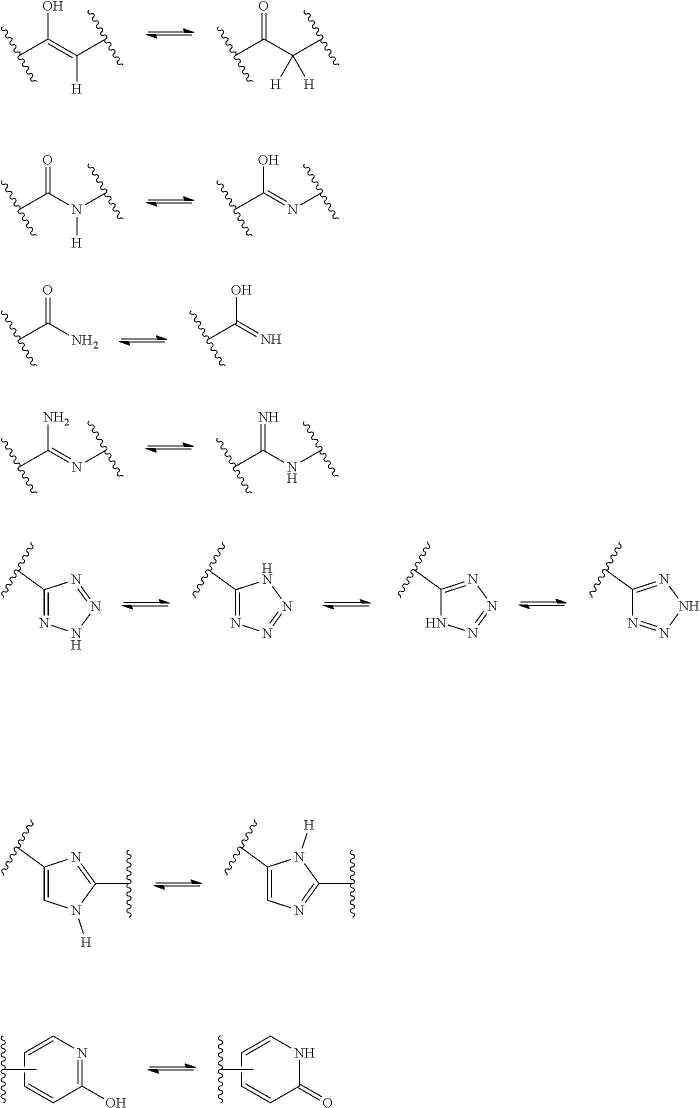

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. In some embodiments, isotopic substitution with ¹⁸F is contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

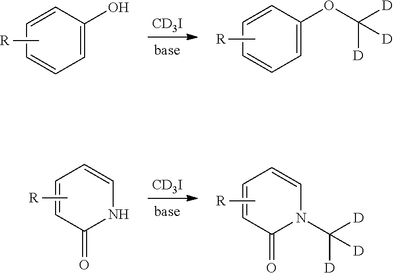

Deuterium-transfer reagents, such as lithium aluminum deuteride (LiAlD₄), are employed to transfer deuterium under reducing conditions to the reaction substrate. The use of LiAlD₄ is illustrated, by way of example only, in the reaction schemes below.

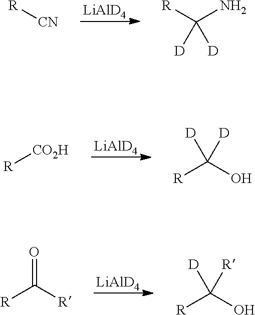

Deuterium gas and palladium catalyst are employed to reduce unsaturated carbon-carbon linkages and to perform a reductive substitution of aryl carbon-halogen bonds as illustrated, by way of example only, in the reaction schemes below.

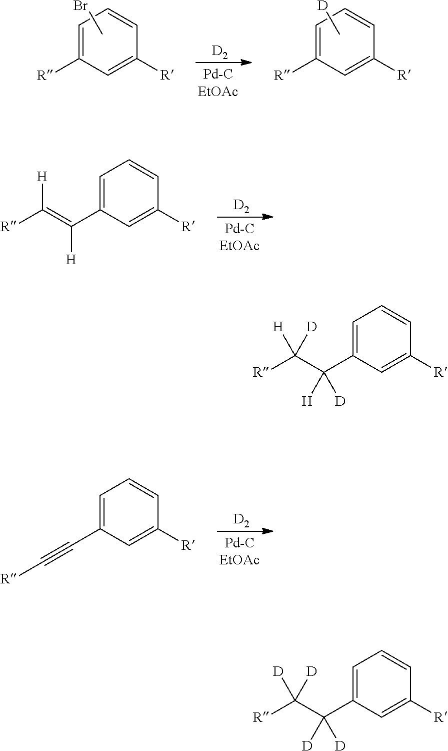

In one embodiment, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain more than six deuterium atoms. In another embodiment, the compound disclosed herein is fully substituted with deuterium atoms and contains no non-exchangeable ¹H hydrogen atoms. In one embodiment, the level of deuterium incorporation is determined by synthetic methods in which a deuterated synthetic building block is used as a starting material.

“Pharmaceutically acceptable salt” includes both acid and base addition salts. A pharmaceutically acceptable salt of any one of the inhibitor of fibroblast growth factor receptors (FGFRs) compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Pharmaceutically acceptable solvate” refers to a composition of matter that is the solvent addition form. In some embodiments, solvates contain either stoichiometric or non-stoichiometric amounts of a solvent, and are formed during the process of making with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. The compounds provided herein optionally exist in either unsolvated as well as solvated forms. The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

### Fibroblast Growth Factor Receptor (FGFR)

Fibroblast growth factor receptors (FGFRs) are a subfamily of receptor tyrosine kinases (RTKs) that bind to members of the fibroblast growth factor family of proteins. FGFR genes generally contain 18 exons, possess similar exon-intron organization, and are randomly dispersed throughout the genome with no apparent linkages to FGF gene locations. FGFRs are differentially expressed in a tissue-specific manner throughout development and into adulthood and comprise an extracellular ligand-binding domain, a single-transmembrane domain, and a split intracellular kinase domain. The extracellular region contains two to three immunoglobulin (Ig)-like domains that are involved in FGF binding. These Ig-like domains regulate both ligand affinity and ligand specificity. The intracellular region has the functional domain responsible for FGFR tyrosine kinase activity, as well as additional sites that play a role in protein binding and phosphorylation or autophosphorylation of the receptor molecule. Fibroblast grouth factor receptor pharmacology has been reviewed in the scientific literature by Porta et al. (Criticial Reviews in Oncology/Hematology 113 (2017) 256-67) and Babina and Turner (Nature Review-Cancer 2017 doi: 10.1038/nrc.2017.8).

The FGFR family comprises of four family members—FGFR1, FGFR2, FGFR3, and FGFR4, but the four members are capable of producing multiple receptor isoforms through alternative splicing of primary transcripts. A closely-related receptor which lacks the FGF signaling tyrosine kinase domain, FGFR5, (also known as FGFRL1) was recently discovered on the basis of interaction with FGFR-binding ligands, known as fibroblast growth factors (FGFs) (Trueb B. Biology of FGFRL1, the fifth fibroblast growth factor receptor. Cell Mol Life Sci. 2011; 68(6):951-964). Collectively, FGFR signaling is associated with the activation of multiple cellular cascades and responses such as cell growth, proliferation, differentiation, and survival (Thisse B et al. Functions and regulations of fibroblast growth factor signaling during embryonic development. Dev Biol. 2005; 287(2):390-402; Wesche J et al. Fibroblast growth factors and their receptors in cancer. Biochem J. 2011; 437(2):199-213; Haugsten E M et al. Roles of fibroblast growth factor receptors in carcinogenesis. Mol Cancer Res. 2010; 8(11):1439-1452).

Numerous human pathological conditions are associated with the deregulation of FGFR signaling. Aberrant FGFR signaling is largely attributed to several underlying mechanisms involving gene amplification, gain-of-function coding mutation, gene fusions, single nucleotide polymorphism (SNP), ligand availability and impaired termination program in FGF-mediated signaling (Tiong K H et al. Functional roles of fibroblast growth factor receptors (FGFRs) signaling in human cancers. Apoptosis. 2013; 18(12):1447-68). In addition, a further layer of complexity is added by the fact that FGFRs are subjected to alternative splicing, giving rise to multiple isoforms which may promote or repress tumorigenesis, under different circumstances.

### FGFR Fusions

FGFR fusions in human cancers are classified into type 1 fusions caused by chromosomal translocations in hematological malignancies, and type 2 fusions caused by chromosomal rearrangements in solid tumors (FGFR inhibitors: Effects on cancer cells, tumor microenvironment and whole-body homeostasis (Review). Int J Mol Med. 2016; 38(1):3-15). Both types of FGFR fusion proteins are endowed with oncogenic potential through the acquisition of protein-protein-interaction modules from fusion partners for ligand-independent dimerization and/or recruitment of aberrant substrates. Human FGFR fusion proteins generally consist of two main segments—the anterior being a dimerized domain from a partnering gene and tyrosine kinase domain at the posterior (Garcia-Closas M et al. Heterogeneity of breast cancer associations with five susceptibility loci by clinical and pathological characteristics. PLoS Genet. 2008; 4(4):e1000054). Unlike wild type receptors, mutant FGFRs are expressed intracellularly and retained in the cytosol, thus they escape the typical receptor degradation processes, further prolonging the activation signal.

Deregulation of the fibroblast growth factor (FGF)/FGF receptor (FGFR) network occurs frequently in tumors, resulting in the development of FGF/FGFR-targeting therapies as the focus of several basic, preclinical, and clinical studies.

### Heteroaromatic FGFR Inhibitory Compounds

In one aspect, provided herein is a heteroaromatic FGFR inhibitory compound.

One embodiment provides a compound, or pharmaceutically acceptable salt or solvate thereof, having the structure of Formula (I):

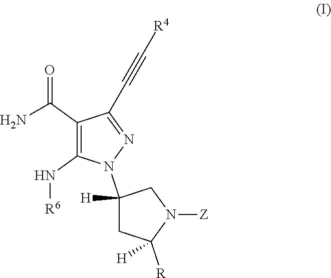

wherein,
Z is selected from a group having the structure:

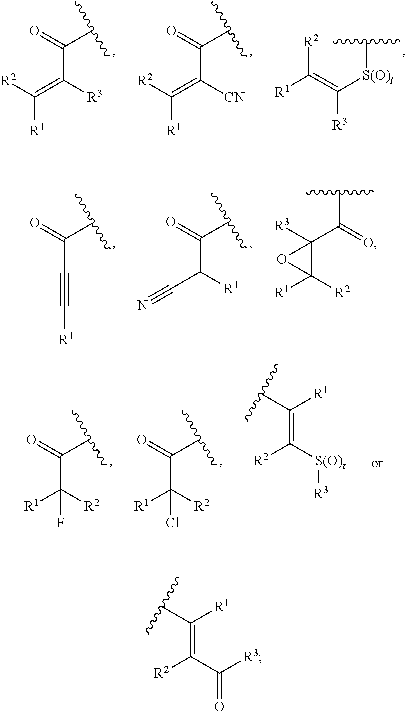

t is 1 or 2;
R¹, R², and R³ are each independently selected from hydrogen, fluoro, optionally substituted C1-C4 alkyl, or optional substituted heterocyclylalkyl;
R⁴ is an optionally substituted nitrogen-containing 9 or 10-atom heteroaryl;
R is selected from hydrogen, optionally substituted C1-C6 alkyl, optionally substituted C3-C7 carbocyclyl, optionally substituted C3-C7 carbocyclylalkyl, optionally substituted C3-C7 heterocyclyl, optionally substituted C3-C7 heterocyclylalkyl, optionally substituted C2-C7 alkenyl, —CO₂R⁵, —CONHR⁵, or —CON(R⁵)₂;
each R⁵ is independently selected from optionally substituted C1-C6 alkyl, optionally substituted C3-C7 carbocyclyl, optionally substituted C3-C7 carbocyclylalkyl, optionally substituted C3-C7 heterocyclyl, or optionally substituted C3-C7 heterocyclylalkyl; and
R⁵ is an optionally substituted alkyl, optionally substituted carbocyclylalkyl, or optionally substituted heterocyclylalkyl.

Another embodiment provides the compound of Formula (I), or a pharmaceutically acceptable salt or solvate thereof, wherein Z is

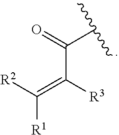

Another embodiment provides the compound, or pharmaceutically acceptable salt or solvate thereof, wherein R² is hydrogen. Another embodiment provides the compound, or pharmaceutically acceptable salt or solvate thereof, wherein R³ is hydrogen or fluoro. Another embodiment provides the compound, or pharmaceutically acceptable salt or solvate thereof, wherein R² and R³ are hydrogen. Another embodiment provides the compound, or pharmaceutically acceptable salt or solvate thereof, wherein R¹ is hydrogen. Another embodiment provides the compound, or pharmaceutically acceptable salt or solvate thereof, wherein R¹ is optionally substituted C1-C4 alkyl. Another embodiment provides the compound, or pharmaceutically acceptable salt or solvate thereof, wherein R¹ is optionally substituted C1-C2 alkyl. Another embodiment provides the compound, or pharmaceutically acceptable salt or solvate thereof, wherein R¹ is optionally substituted C1 alkyl. Another embodiment provides the compound, or pharmaceutically acceptable salt or solvate thereof, wherein the optionally substituted alkyl is substituted with an optionally substituted amino group. Another embodiment provides the compound, or pharmaceutically acceptable salt or solvate thereof, wherein the optionally substituted amino group is a dimethylamino.

Another embodiment provides the compound of Formula (I), or a pharmaceutically acceptable salt or solvate thereof, wherein R⁴ is selected from an optionally substituted nitrogen-containing 9 or 10-atom heteroaryl is selected from optionally substituted benzimidazole, optionally substituted 1H-indazole, optionally substituted 2H-indazole, optionally substituted benzotriazole, optionally substituted benzoxazole, optionally substituted imidazo[4,5-c]pyridine, or optionally substituted imidazo[4,5-b]pyridine. Another embodiment provides the compound of Formula (I), or a pharmaceutically acceptable salt or solvate thereof, wherein R⁴ is selected from an optionally substituted nitrogen-containing 9 or 10-atom heteroaryl is selected from quinoline, quinoxaline, pyrazolo[1,5-a]pyrimidine, imidazo[1,2-a]pyridine, pyrazolo[1,5-a]pyridine, imidazo[1,2-a]pyrimidine, imidazo[1,2-b]pyridazine, or pyrazolo[1,5-a]pyridine. Another embodiment provides the compound, or a pharmaceutically acceptable salt or solvate thereof, wherein R⁴ is an optionally substituted benzimidazole. Another embodiment provides the compound, or a pharmaceutically acceptable salt or solvate thereof, wherein R⁴ is an optionally substituted 1H-indazole. Another embodiment provides the compound, or a pharmaceutically acceptable salt or solvate thereof, wherein R⁴ is an optionally substituted 2H-indazole. Another embodiment provides the compound, or a pharmaceutically acceptable salt or solvate thereof, wherein R⁴ is an optionally substituted benzoxazole, optionally substituted imidazo[4,5-c]pyridine, or optionally substituted imidazo[4,5-b]pyridine. Another embodiment provides the compound, or a pharmaceutically acceptable salt or solvate thereof, wherein optionally substituted nitrogen-containing 9 or 10-atom heteroaryl is optionally substituted with alkyl, cycloalkyl, or halogen. Another embodiment provides the compound, or a pharmaceutically acceptable salt or solvate thereof, wherein the optionally substituted benzimidazole is optionally substituted with alkyl, cycloalkyl, or halogen. Another embodiment provides the compound, or a pharmaceutically acceptable salt or solvate thereof, wherein the optionally substituted 1H-indazole is optionally substituted with alkyl, cycloalkyl, or halogen. Another embodiment provides the compound, or a pharmaceutically acceptable salt or solvate thereof, wherein the optionally substituted 2H-indazole is optionally substituted with alkyl, cycloalkyl, or halogen.

Another embodiment provides the compound of Formula (I), or a pharmaceutically acceptable salt or solvate thereof, wherein R is hydrogen. Another embodiment provides the compound, or a pharmaceutically acceptable salt or solvate thereof, wherein R is optionally substituted C1-C6 alkyl. Another embodiment provides the compound, or a pharmaceutically acceptable salt or solvate thereof, wherein R is optionally substituted C3-C7 carbocyclyl. Another embodiment provides the compound, or a pharmaceutically acceptable salt or solvate thereof, wherein R is optionally substituted C3-C7 carbocyclylalkyl. Another embodiment provides the compound, or a pharmaceutically acceptable salt or solvate thereof, wherein R is optionally substituted C3-C7 heterocyclyl. Another embodiment provides the compound, or a pharmaceutically acceptable salt or solvate thereof, wherein R is optionally substituted C3-C7 heterocyclylalkyl. Another embodiment provides the compound, or a pharmaceutically acceptable salt or solvate thereof, wherein R is —CO₂R⁵. Another embodiment provides the compound, or a pharmaceutically acceptable salt or solvate thereof, wherein R is —CONHR⁵ or —CON(R⁵)₂. Another embodiment provides the compound, or a pharmaceutically acceptable salt or solvate thereof, wherein the optionally substituted C1-C6 alkyl is a C1-C3 alkyl substituted with a C1-C3 alkoxy.

Another embodiment provides the compound of Formula (I), or a pharmaceutically acceptable salt or solvate thereof, wherein R⁶ is an optionally substituted alkyl. Another embodiment provides the compound, or a pharmaceutically acceptable salt or solvate thereof, wherein R⁶ is an optionally substituted C1-C4 alkyl. Another embodiment provides the compound, or a pharmaceutically acceptable salt or solvate thereof, wherein R⁶ is an optionally substituted C1-C3 alkyl. Another embodiment provides the compound, or a pharmaceutically acceptable salt or solvate thereof, wherein R⁶ is an optionally substituted C1-C2 alkyl. Another embodiment provides the compound, or a pharmaceutically acceptable salt or solvate thereof, wherein R⁶ is an optionally substituted C1 alkyl. Another embodiment provides the compound, or a pharmaceutically acceptable salt or solvate thereof, wherein R⁶ is CH₃.

Another embodiment provides the compound of Formula (I), or a pharmaceutically acceptable salt or solvate thereof, wherein R⁶ is an optionally substituted carbocyclylalkyl. Another embodiment provides the compound, or a pharmaceutically acceptable salt or solvate thereof, wherein R⁶ is optionally substituted carbocyclylmethyl. Another embodiment provides the compound, or a pharmaceutically acceptable salt or solvate thereof, wherein R⁶ is optionally substituted (C3-C6carbocyclyl)methyl. Another embodiment provides the compound, or a pharmaceutically acceptable salt or solvate thereof, wherein R⁶ is optionally substituted cyclopropylmethyl. Another embodiment provides the compound of Formula (I), or a pharmaceutically acceptable salt or solvate thereof, wherein R⁶ is an optionally substituted heterocyclylalkyl. Another embodiment provides the compound, or a pharmaceutically acceptable salt or solvate thereof, wherein R⁶ is optionally substituted heterocyclylethyl. Another embodiment provides the compound, or a pharmaceutically acceptable salt or solvate thereof, wherein R⁶ is optionally substituted heterocyclylethyl and the heterocyclyl is a piperidinyl, pyrrolidinyl, morpholinyl, or piperazinyl.

Another embodiment provides the compound of Formula (I), or a pharmaceutically acceptable salt or solvate thereof, wherein R⁴ is an optionally substituted benzimidazole further substituted with a cycloalkyl group, and at least one halogen.

Another embodiment provides the compound of Formula (I), or a pharmaceutically acceptable salt or solvate thereof, wherein R⁴ is an optionally substituted benzimidazole further substituted with a cycloalkyl group, and at least one halogen; R is a —CH₂OCH₃ group; and R⁶ is methyl.

Another embodiment provides the compound of Formula (I), or a pharmaceutically acceptable salt or solvate thereof, wherein R⁴ is an optionally substituted benzimidazole further substituted with a cycloalkyl group, and at least one halogen; R is a hydrogen; and R⁶ is methyl.

One embodiment provides a compound, or pharmaceutically acceptable salt or solvate thereof, having the structure of Formula (II):

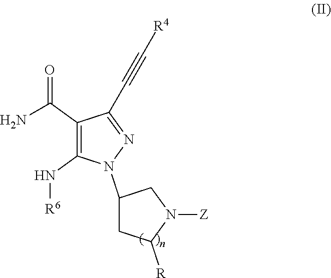

wherein,
n is 0 or 1;
Z is selected from a group having the structure:

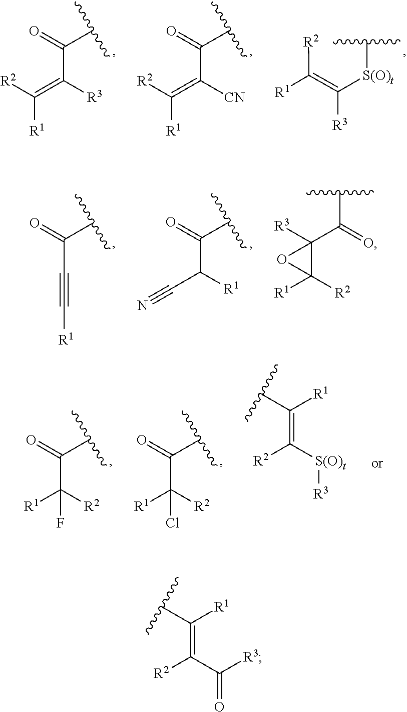

t is 1 or 2;
R¹, R², and R³ are each independently selected from hydrogen, fluoro, optionally substituted C1-C4 alkyl, or optional substituted heterocyclylalkyl;
R⁴ is an optionally substituted nitrogen-containing 9 or 10-atom heteroaryl;
R is selected from hydrogen, optionally substituted C1-C6 alkyl, optionally substituted C3-C7 carbocyclyl, optionally substituted C3-C7 carbocyclylalkyl, optionally substituted C3-C7 heterocyclyl, optionally substituted C3-C7 heterocyclylalkyl, optionally substituted C2-C7 alkenyl, —CO₂R⁵, —CONHR⁵, or —CON(R⁵)₂;
each R⁵ is independently selected from optionally substituted C1-C6 alkyl, optionally substituted C3-C7 carbocyclyl, optionally substituted C3-C7 carbocyclylalkyl, optionally substituted C3-C7 heterocyclyl, or optionally substituted C3-C7 heterocyclylalkyl; and
R⁶ is hydrogen, optionally substituted alkyl, optionally substituted carbocyclyl, optionally substituted carbocyclylalkyl, or optionally substituted heterocyclylalkyl.

Another embodiment provides the compound of Formula (II), or a pharmaceutically acceptable salt or solvate thereof, wherein n is 0.

Another embodiment provides the compound of Formula (II), or a pharmaceutically acceptable salt or solvate thereof, wherein n is 1. Another embodiment provides the compound of Formula (II), or a pharmaceutically acceptable salt or solvate thereof, wherein n is 1 and the stereochemistry across the pyrrolidine ring is cis. Another embodiment provides the compound of Formula (II), or a pharmaceutically acceptable salt or solvate thereof, wherein n is 1 and the stereochemistry across the pyrrolidine ring is trans.

Another embodiment provides the compound of Formula (II), or a pharmaceutically acceptable salt or solvate thereof, wherein Z is

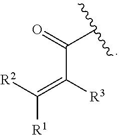

Another embodiment provides the compound, or pharmaceutically acceptable salt or solvate thereof, wherein R² is hydrogen. Another embodiment provides the compound, or pharmaceutically acceptable salt or solvate thereof, wherein R³ is hydrogen or fluoro. Another embodiment provides the compound, or pharmaceutically acceptable salt or solvate thereof, wherein R² and R³ are hydrogen. Another embodiment provides the compound, or pharmaceutically acceptable salt or solvate thereof, wherein R¹ is hydrogen. Another embodiment provides the compound, or pharmaceutically acceptable salt or solvate thereof, wherein R¹ is optionally substituted C1-C4 alkyl. Another embodiment provides the compound, or pharmaceutically acceptable salt or solvate thereof, wherein R¹ is optionally substituted C1-C2 alkyl. Another embodiment provides the compound, or pharmaceutically acceptable salt or solvate thereof, wherein R¹ is optionally substituted C1 alkyl. Another embodiment provides the compound, or pharmaceutically acceptable salt or solvate thereof, wherein the optionally substituted alkyl is substituted with an optionally substituted amino group. Another embodiment provides the compound, or pharmaceutically acceptable salt or solvate thereof, wherein the optionally substituted amino group is a dimethylamino.

Another embodiment provides the compound of Formula (II), or a pharmaceutically acceptable salt or solvate thereof, wherein R⁴ is selected from an optionally substituted nitrogen-containing 9 or 10-atom heteroaryl is selected from optionally substituted benzimidazole, optionally substituted 1H-indazole, optionally substituted 2H-indazole, optionally substituted benzotriazole, optionally substituted benzoxazole, optionally substituted imidazo[4,5-c]pyridine, or optionally substituted imidazo[4,5-b]pyridine. Another embodiment provides the compound of Formula (II), or a pharmaceutically acceptable salt or solvate thereof, wherein R⁴ is selected from an optionally substituted nitrogen-containing 9 or 10-atom heteroaryl is selected from quinoline, quinoxaline, pyrazolo[1,5-a]pyrimidine, imidazo[1,2-a]pyridine, pyrazolo[1,5-a]pyridine, imidazo[1,2-a]pyrimidine, imidazo[1,2-b]pyridazine, or pyrazolo[1,5-a]pyridine. Another embodiment provides the compound, or a pharmaceutically acceptable salt or solvate thereof, wherein R⁴ is an optionally substituted benzimidazole. Another embodiment provides the compound, or a pharmaceutically acceptable salt or solvate thereof, wherein R⁴ is an optionally substituted 1H-indazole. Another embodiment provides the compound, or a pharmaceutically acceptable salt or solvate thereof, wherein R⁴ is an optionally substituted 2H-indazole. Another embodiment provides the compound, or a pharmaceutically acceptable salt or solvate thereof, wherein R⁴ is an optionally substituted benzoxazole, optionally substituted imidazo[4,5-c]pyridine, or optionally substituted imidazo[4,5-b]pyridine. Another embodiment provides the compound, or a pharmaceutically acceptable salt or solvate thereof, wherein optionally substituted nitrogen-containing 9 or 10-atom heteroaryl is optionally substituted with alkyl, cycloalkyl, or halogen. Another embodiment provides the compound, or a pharmaceutically acceptable salt or solvate thereof, wherein the optionally substituted benzimidazole is optionally substituted with alkyl, cycloalkyl, or halogen. Another embodiment provides the compound, or a pharmaceutically acceptable salt or solvate thereof, wherein the optionally substituted 1H-indazole is optionally substituted with alkyl, cycloalkyl, or halogen. Another embodiment provides the compound, or a pharmaceutically acceptable salt or solvate thereof, wherein the optionally substituted 2H-indazole is optionally substituted with alkyl, cycloalkyl, or halogen.

Another embodiment provides the compound of Formula (II), or a pharmaceutically acceptable salt or solvate thereof, wherein R is hydrogen. Another embodiment provides the compound, or a pharmaceutically acceptable salt or solvate thereof, wherein R is optionally substituted C1-C6 alkyl. Another embodiment provides the compound, or a pharmaceutically acceptable salt or solvate thereof, wherein R is optionally substituted C3-C7 carbocyclyl. Another embodiment provides the compound, or a pharmaceutically acceptable salt or solvate thereof, wherein R is optionally substituted C3-C7 carbocyclylalkyl. Another embodiment provides the compound, or a pharmaceutically acceptable salt or solvate thereof, wherein R is optionally substituted C3-C7 heterocyclyl. Another embodiment provides the compound, or a pharmaceutically acceptable salt or solvate thereof, wherein R is optionally substituted C3-C7 heterocyclylalkyl. Another embodiment provides the compound, or a pharmaceutically acceptable salt or solvate thereof, wherein R is —CO₂R⁵. Another embodiment provides the compound, or a pharmaceutically acceptable salt or solvate thereof, wherein R is —CONHR⁵ or —CON(R⁵)₂. Another embodiment provides the compound, or a pharmaceutically acceptable salt or solvate thereof, wherein the optionally substituted C1-C6 alkyl is a C1-C3 alkyl substituted with a C1-C3 alkoxy.

Another embodiment provides the compound of Formula (II), or a pharmaceutically acceptable salt or solvate thereof, wherein R⁶ is hydrogen.

Another embodiment provides the compound of Formula (II), or a pharmaceutically acceptable salt or solvate thereof, wherein R⁶ is an optionally substituted alkyl. Another embodiment provides the compound, or a pharmaceutically acceptable salt or solvate thereof, wherein R⁶ is an optionally substituted C1-C4 alkyl. Another embodiment provides the compound, or a pharmaceutically acceptable salt or solvate thereof, wherein R⁶ is an optionally substituted C1-C3 alkyl. Another embodiment provides the compound, or a pharmaceutically acceptable salt or solvate thereof, wherein R⁶ is an optionally substituted C1-C2 alkyl. Another embodiment provides the compound, or a pharmaceutically acceptable salt or solvate thereof, wherein R⁶ is an optionally substituted C1 alkyl. Another embodiment provides the compound, or a pharmaceutically acceptable salt or solvate thereof, wherein R⁶ is CH₃.

Another embodiment provides the compound of Formula (II), or a pharmaceutically acceptable salt or solvate thereof, wherein R⁶ is an optionally substituted carbocyclylalkyl. Another embodiment provides the compound, or a pharmaceutically acceptable salt or solvate thereof, wherein R⁶ is optionally substituted carbocyclylmethyl. Another embodiment provides the compound, or a pharmaceutically acceptable salt or solvate thereof, wherein R⁶ is optionally substituted (C3-C6carbocyclyl)methyl. Another embodiment provides the compound, or a pharmaceutically acceptable salt or solvate thereof, wherein R⁶ is optionally substituted cyclopropylmethyl. Another embodiment provides the compound of Formula (II), or a pharmaceutically acceptable salt or solvate thereof, wherein R⁶ is an optionally substituted heterocyclylalkyl. Another embodiment provides the compound, or a pharmaceutically acceptable salt or solvate thereof, wherein R⁶ is optionally substituted heterocyclylethyl. Another embodiment provides the compound, or a pharmaceutically acceptable salt or solvate thereof, wherein R⁶ is optionally substituted heterocyclylethyl and the heterocyclyl is a piperidinyl, pyrrolidinyl, morpholinyl, or piperazinyl.

Another embodiment provides the compound of Formula (II), or a pharmaceutically acceptable salt or solvate thereof, wherein R⁴ is an optionally substituted benzimidazole further substituted with a cycloalkyl group, and at least one halogen.

Another embodiment provides the compound of Formula (II), or a pharmaceutically acceptable salt or solvate thereof, wherein R⁴ is an optionally substituted benzimidazole further substituted with a cycloalkyl group, and at least one halogen; R is a —CH₂OCH₃ group; and R⁶ is methyl.

Another embodiment provides the compound of Formula (II), or a pharmaceutically acceptable salt or solvate thereof, wherein R⁴ is an optionally substituted benzimidazole further substituted with a cycloalkyl group, and at least one halogen; R is a hydrogen; and R⁶ is methyl.

In some embodiments, the heteroaromatic FGFR kinase inhibitory compound disclosed herein has a structure provided in Table 1.

TABLE 1

| Synthetic | | |
|---|---|---|
| Chemistry | | |
| Example | Compound Structure | Compound Name |
| 1 | 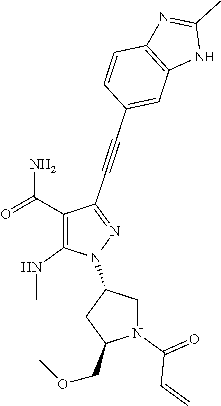 [SMILES: C=CC(=O)N1C[C@@H](n2nc(C#Cc3ccc4nc(C)[nH]c4c3)c(C(N)=O)c2NC)C[C@@H]1COC] | 1-[(3S,5R)-5-(methoxymethyl)-1- (prop-2-enoyl)pyrrolidin-3-yl]-3-[2-(2- methyl-3H-1,3-benzodiazol-5- yl)ethynyl]-5-(methylamino)pyrazole- 4-carboxamide |
| 2 | 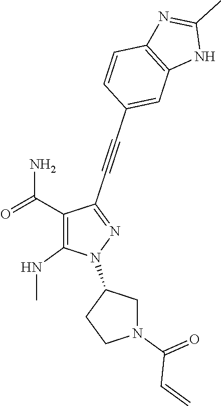 [SMILES: C=CC(=O)N1CC[C@H](n2nc(C#Cc3ccc4nc(C)[nH]c4c3)c(C(N)=O)c2NC)C1] | (S)-1-(1-acryloylpyrrolidin-3-yl)-3-((2- methyl-1H-benzo[d]imidazol-6- yl)ethynyl)-5-(methylamino)-1H- pyrazole-4-carboxamide |
| 3 | 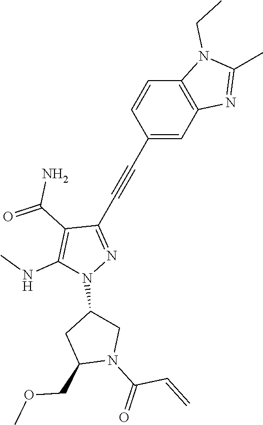 | 3-[2-(1-ethyl-2-methyl-1,3- benzodiazol-5-yl)ethynyl]-1-[(3S,5R)- 5-(methoxymethyl)-1-(prop-2- enoyl)pyrrolidin-3-yl]-5- (methylamino)pyrazole-4-carboxamide |
| 4 | 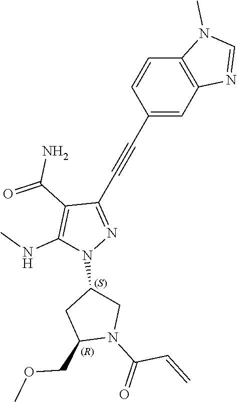 | 1-[(3S,5R)-5-(methoxymethyl)-1- (prop-2-enoyl)pyrrolidin-3-yl]-3-[2-(1- methyl-1,3-benzodiazol-5-yl)ethynyl]- 5-(methylamino)pyrazole-4- carboxamide |
| 5 | 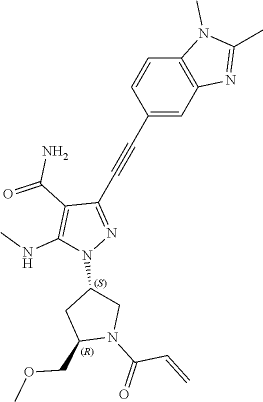 | 1-((3S,5R)-1-acryloyl-5- (methoxymethyl)pyrrolidin-3-yl)-3- ((1,2-dimethyl-1H-benzo[d]imidazol- 5-yl)ethynyl)-5-(methylamino)-1H- pyrazole-4-carboxamide |
| 6 | 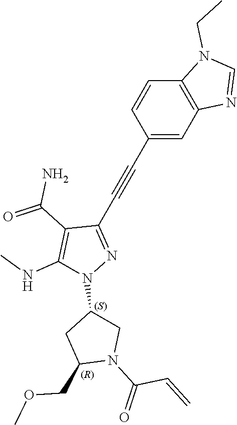 | 1-((3S,5R)-1-acryloyl-5- (methoxymethyl)pyrrolidin-3-yl)-3- ((1-ethyl-1H-benzo[d]imidazol-5- yl)ethynyl)-5-(methylamino)-1H- pyrazole-4-carboxamide |
| 7 | 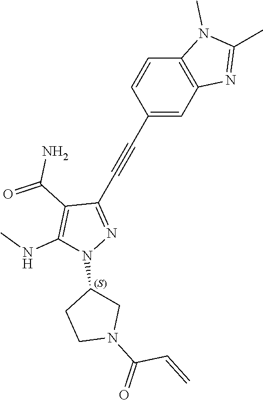 | (S)-1-(1-acryloylpyrrolidin-3-yl)-3- ((1,2-dimethyl-1H-benzo[d]imidazol- 5-yl)ethynyl)-5-(methylamino)-1H- pyrazole-4-carboxamide |
| 8 | 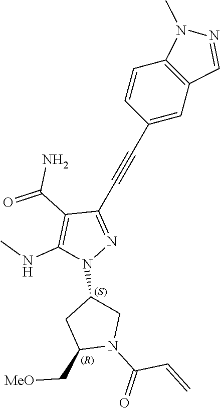 | 1-((3S,5R)-1-acryloyl-5- (methoxymethyl)pyrrolidin-3-yl)-3- ((1-methyl-1H-indazol-5-yl)ethynyl)- 5-(methylamino)-1H-pyrazole-4- carboxamide |
| 9 | 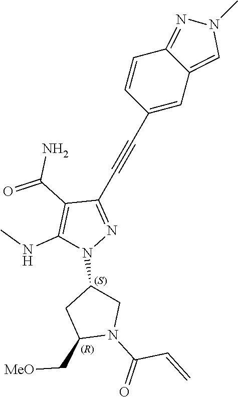 | 1-((3S,5R)-1-acryloyl-5- (methoxymethyl)pyrrolidin-3-yl)-3- ((2-methyl-2H-indazol-5-yl)ethynyl)- 5-(methylamino)-1H-pyrazole-4- carboxamide |
| 10 | 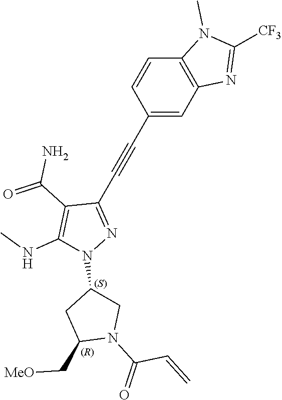 | 1-((3S,5R)-1-acryloyl-5- (methoxymethyl)pyrrolidin-3-yl)-3- ((1-methyl-2-(trifluoromethyl)-1H- benzo[d]imidazol-5-yl)ethynyl)-5- (methylamino)-1H-pyrazole-4- carboxamide |
| 11 | 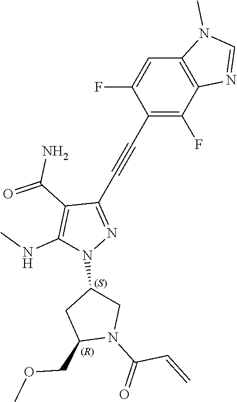 | 1-((3S,5R)-1-acryloyl-5- (methoxymethyl)pyrrolidin-3-yl)-3- ((4,6-difluoro-1-methyl-1H- benzo[d]imidazol-5-yl)ethynyl)-5- (methylamino)-1H-pyrazole-4- carboxamide |
| 12 | 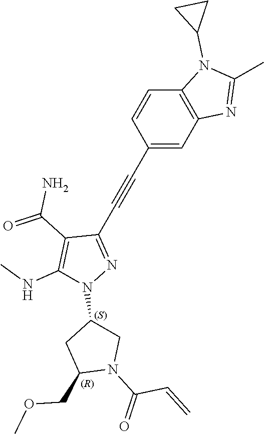 | 1-((3S,5R)-1-acryloyl-5- (methoxymethyl)pyrrolidin-3-yl)-3- ((1-cyclopropyl-2-methyl-1H- benzo[d]imidazol-5-yl)ethynyl)-5- (methylamino)-1H-pyrazole-4- carboxamide |
| 13 | 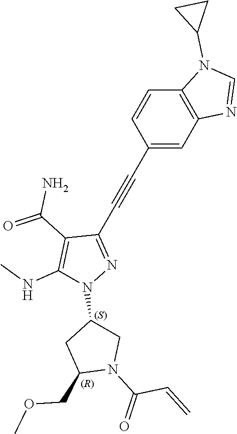 | 1-((3S,5R)-1-acryloyl-5- (methoxymethyl)pyrrolidin-3-yl)-3- ((1-cyclopropyl-1H-benzo[d]imidazol- 5-yl)ethynyl)-5-(methylamino)-1H- pyrazole-4-carboxamide |
| 14 | 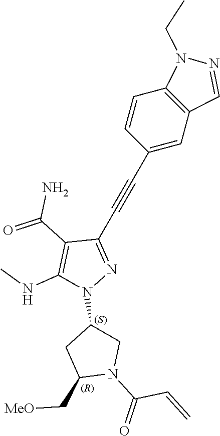 | 1-((3S,5R)-1-acryloyl-5- (methoxymethyl)pyrrolidin-3-yl)-3- ((1-ethyl-1H-indazol-5-yl)ethynyl)-5- (methylamino)-1H-pyrazole-4- carboxamide |
| 15 | 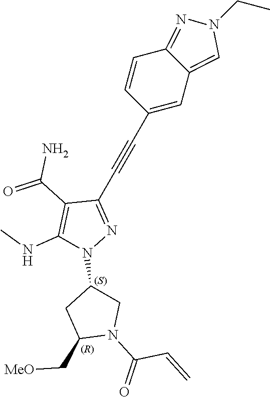 | 1-((3S,5R)-1-acryloyl-5- (methoxymethyl)pyrrolidin-3-yl)-3- ((2-ethyl-2H-indazol-5-yl)ethynyl)-5- (methylamino)-1H-pyrazole-4- carboxamide |
| 16 | 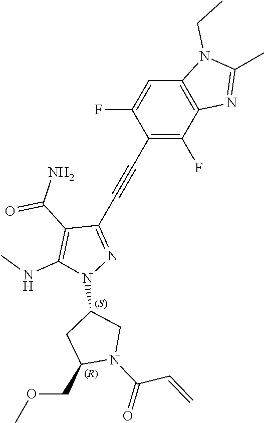 | 1-((3S,5R)-1-acryloyl-5- (methoxymethyl)pyrrolidin-3-yl)-3- ((1-ethyl-4,6-difluoro-2-methyl-1H- benzo[d]imidazol-4-yl)ethynyl)-5- (methylamino)-1H-pyrazole-4- carboxamide |
| 17 | 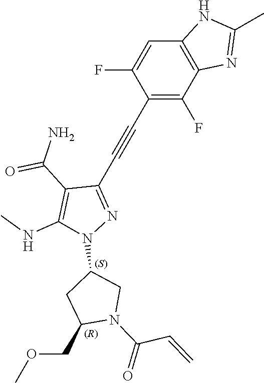 | 1-((3S,5R)-1-acryloyl-5- (methoxymethyl)pyrrolidin-3-yl)-3- ((4,6-difluoro-2-methyl-1H- benzo[d]imidazol-5-yl)ethynyl)-5- (methylamino)-1H-pyrazole-4- carboxamide |
| 18 | 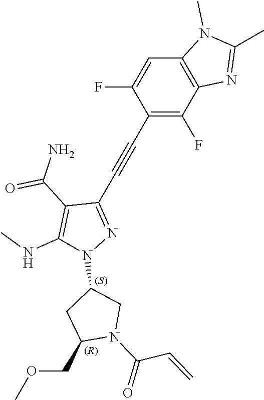 | 1-((3S,5R)-1-acryloyl-5- (methoxymethyl)pyrrolidin-3-yl)-3- ((4,6-difluoro-1,2-dimethyl-1H- benzo[d]imidazol-5-yl)ethynyl)-5- (methylamino)-1H-pyrazole-4- carboxamide |
| 19 | 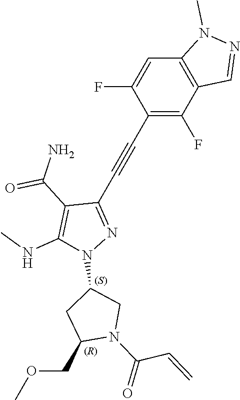 | 1-((3S,5R)-1-acryloyl-5- (methoxymethyl)pyrrolidin-3-yl)-3- ((4,6-difluoro-1-methyl-1H-indazol-5- yl)ethynyl)-5-(methylamino)-1H- pyrazole-4-carboxamide |
| 20 | 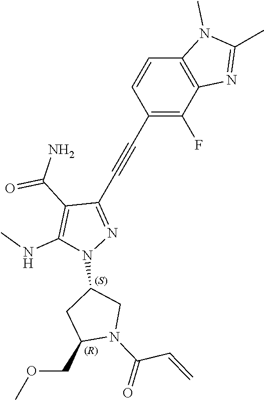 | 1-((3S,5R)-1-acryloyl-5- (methoxymethyl)pyrrolidin-3-yl)-3- ((4-fluoro-1,2-dimethyl-1H- benzo[d]imidazol-5-yl)ethynyl)-5- (methylamino)-1H-pyrazole-4- carboxamide |
| 21 | 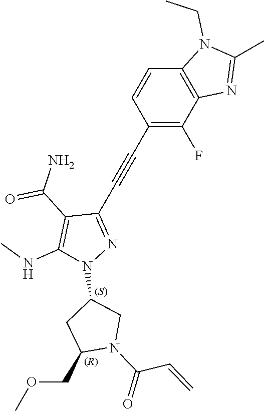 | 1-((3S,5R)-1-acryloyl-5- (methoxymethyl)pyrrolidin-3-yl)-3- ((1-ethyl-4-fluoro-2-methyl-1H- benzo[d]imidazol-5-yl)ethynyl)-5- (methylamino)-1H-pyrazole-4- carboxamide |
| 22 | 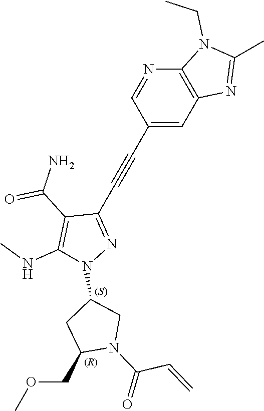 | 1-((3S,5R)-1-acryloyl-5- (methoxymethyl)pyrrolidin-3-yl)-3- ((3-ethyl-2-methyl-3H-imidazo[4,5- b]pyridin-6-yl)ethynyl)-5- (methylamino)-1H-pyrazole-4- carboxamide |
| 23 | 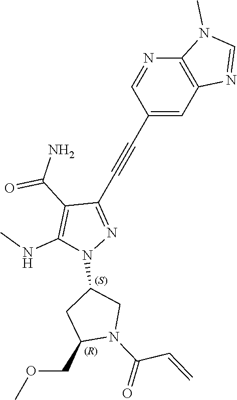 | 1-((3S,5R)-1-acryloyl-5- (methoxymethyl)pyrrolidin-3-yl)-3- ((3-methyl-3H-imidazo[4,5-b]pyridin- 6-yl)ethynyl)-5-(methylamino)-1H- pyrazole-4-carboxamide |
| 24 | 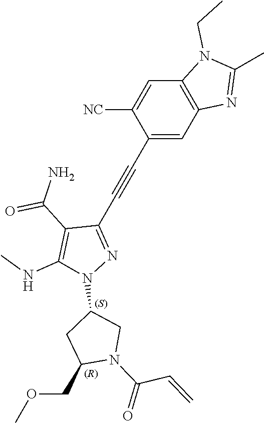 | 1-((3S,5R)-1-acryloyl-5- (methoxymethyl)pyrrolidin-3-yl)-3- ((6-cyano-1-ethyl-2-methyl-1H- benzo[d]imidazol-5-yl)ethynyl)-5- (methylamino)-1H-pyrazole-4- carboxamide |
| 25 | 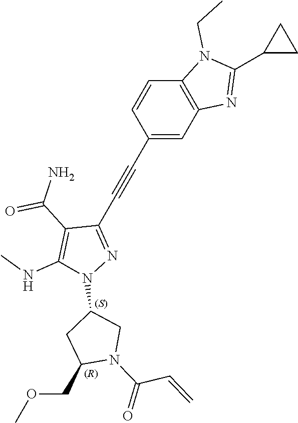 | 1-((3S,5R)-1-acryloyl-5- (methoxymethyl)pyrrolidin-3-yl)-3- ((2-cyclopropyl-1-ethyl-1H- benzo[d]imidazol-5-yl)ethynyl)-5- (methylamino)-1H-pyrazole-4- carboxamide |
| 26 | 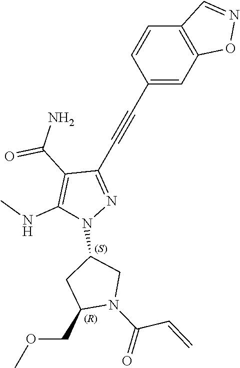 | 1-((3S,5R)-1-acryloyl-5- (methoxymethyl)pyrrolidin-3-yl)-3- (benzo[d]isoxazol-6-ylethynyl)-5- (methylamino)-1H-pyrazole-4- carboxamide |
| 27 | 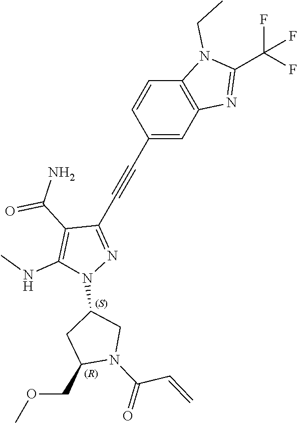 | 1-((3S,5R)-1-acryloyl-5- (methoxymethyl)pyrrolidin-3-yl)-3- ((1-ethyl-2-(trifluoromethyl)-1H- benzo[d]imidazol-5-yl)ethynyl)-5- (methylamino)-1H-pyrazole-4- carboxamide |
| 28 | 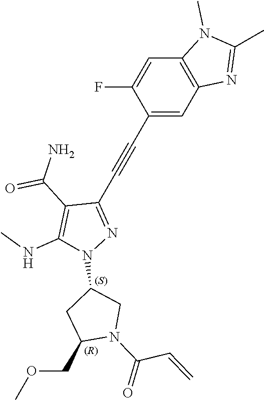 | 1-((3S,5R)-1-acryloyl-5- (methoxymethyl)pyrrolidin-3-yl)-3- ((6-fluoro-1,2-dimethyl-1H- benzo[d]imidazol-5-yl)ethynyl)-5- (methylamino)-1H-pyrazole-4- carboxamide |
| 29 | 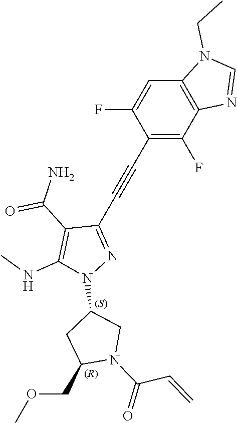 | 1-((3S,5R)-1-acryloyl-5- (methoxymethyl)pyrrolidin-3-yl)-3- ((1-ethyl-4,6-difluoro-1H- benzo[d]imidazol-5-yl)ethynyl)-5- (methylamino)-1H-pyrazole-4- carboxamide |
| 30 | 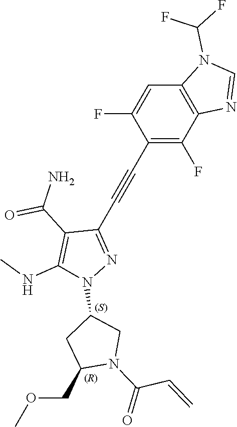 | 1-((3S,5R)-1-acryloyl-5- (methoxymethyl)pyrrolidin-3-yl)-3- ((1-(difluoromethyl)-4,6-difluoro-1H- benzo[d]imidazol-5-yl)ethynyl)-5- (methylamino)-1H-pyrazole-4- carboxamide |
| 31 | 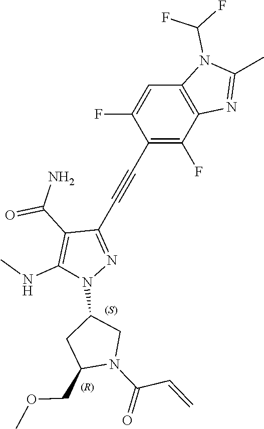 | 1-((3S,5R)-1-acryloyl-5- (methoxymethyl)pyrrolidin-3-yl)-3- ((1-(difluoromethyl)-4,6-difluoro-2- methyl-1H-benzo[d]imidazol-5- yl)ethynyl)-5-(methylamino)-1H- pyrazole-4-carboxamide |
| 32 | 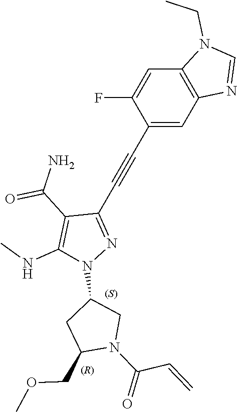 | 1-((3S,5R)-1-acryloyl-5- (methoxymethyl)pyrrolidin-3-yl)-3- ((1-ethyl-6-fluoro-1H- benzo[d]imidazol-5-yl)ethynyl)-5- (methylamino)-1H-pyrazole-4- carboxamide |
| 33 | 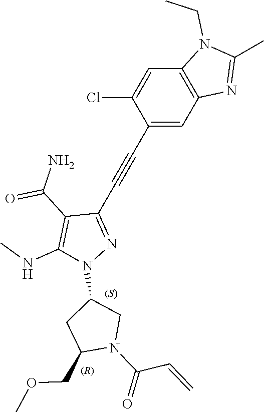 | 1-((3S,5R)-1-acryloyl-5- (methoxymethyl)pyrrolidin-3-yl)-3- ((6-chloro-1-ethyl-2-methyl-1H- benzo[d]imidazol-5-yl)ethynyl)-5- (methylamino)-1H-pyrazole-4- carboxamide |
| 34 | 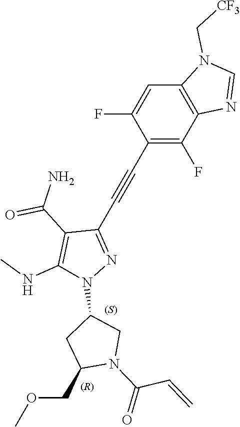 | 1-((3S,5R)-1-acryloyl-5- (methoxymethyl)pyrrolidin-3-yl)-3- ((4,6-difluoro-1-(2,2,2-trifluoroethyl)- 1H-benzo[d]imidazol-5-yl)ethynyl)-5- (methylamino)-1H-pyrazole-4- carboxamide |
| 35 | 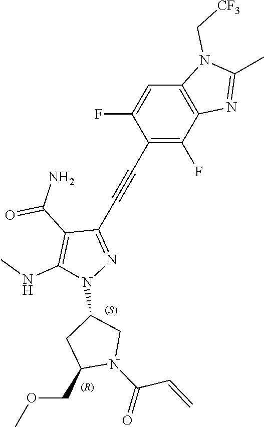 | 1-((3S,5R)-1-acryloyl-5- (methoxymethyl)pyrrolidin-3-yl)-3- ((4,6-difluoro-2-methyl-1-(2,2,2- trifluoroethyl)-1H-benzo[d]imidazol-5- yl)ethynyl)-5-(methylamino)-1H- pyrazole-4-carboxamide |
| 36 | 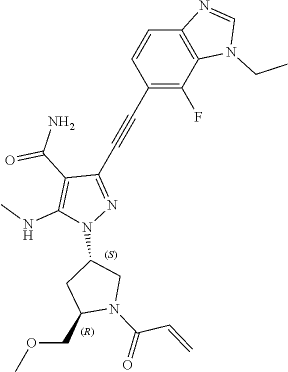 | 3-[2-(3-ethyl-4-fluoro-1,3- benzodiazol-5-yl)ethynyl]-1-[(3S,5R)- 5-(methoxymethyl)-1-(prop-2- enoyl)pyrrolidin-3-yl]-5- (methylamino)pyrazole-4-carboxamide |
| 37 | 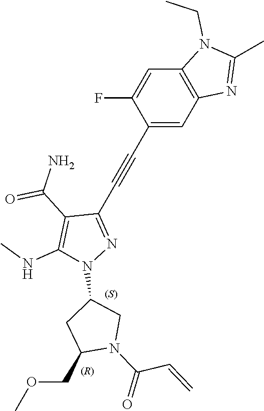 | 3-[2-(1-ethyl-6-fluoro-2-methyl-1,3- benzodiazol-5-yl)ethynyl]-1-[(3S,5R)- 5-(methoxymethyl)-1-(prop-2- enoyl)pyrrolidin-3-yl]-5- (methylamino)pyrazole-4-carboxamide |
| 38 | 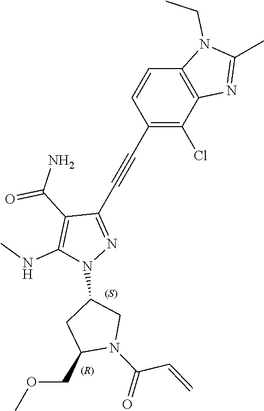 | 3-[2-(4-chloro-1-ethyl-2-methyl-1,3- benzodiazol-5-yl)ethynyl]-1-[(3S,5R)- 5-(methoxymethyl)-1-(prop-2- enoyl)pyrrolidin-3-yl]-5- (methylamino)pyrazole-4-carboxamide |
| 39 | 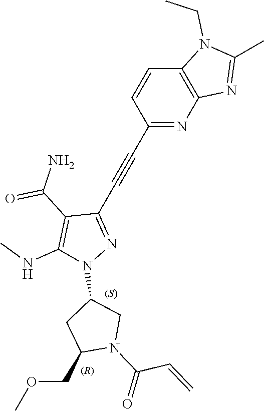 | 3-(2-[1-ethyl-2-methylimidazo[4,5- b]pyridin-5-yl]ethynyl)-1-[(3S,5R)-5- (methoxymethyl)-1-(prop-2- enoyl)pyrrolidin-3-yl]-5- (methylamino)pyrazole-4-carboxamide |
| 40 | 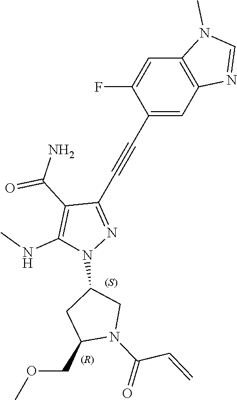 | 3-[2-(6-fluoro-2-methyl-1,3- benzodiazol-5-yl)ethynyl]-1-[(3S,5R)- 5-(methoxymethyl)-1-(prop-2- enoyl)pyrrolidin-3-yl]-5- (methylamino)pyrazole-4-carboxamide |
| 41 | 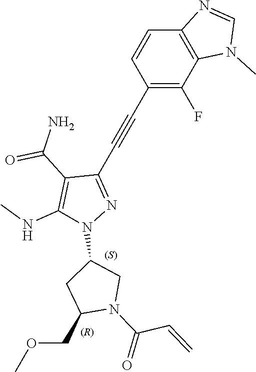 | 3-[2-(4-fluoro-1-methyl-1,3- benzodiazol-5-yl)ethynyl]-1-[(3S,5R)- 5-(methoxymethyl)-1-(prop-2- enoyl)pyrrolidin-3-yl]-5- (methylamino)pyrazole-4-carboxamide |
| 42 | 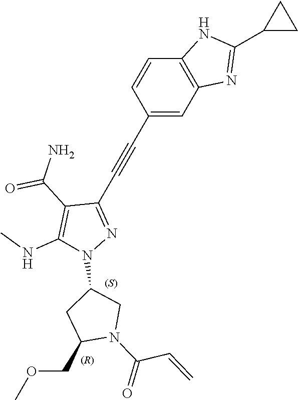 | 3-[2-(2-cyclopropyl-1H-1,3- benzodiazol-5-yl)ethynyl]-1-[(3S,5R)- 5-(methoxymethyl)-1-(prop-2- enoyl)pyrrolidin-3-yl]-5- (methylamino)pyrazole-4-carboxamide |
| 43 | 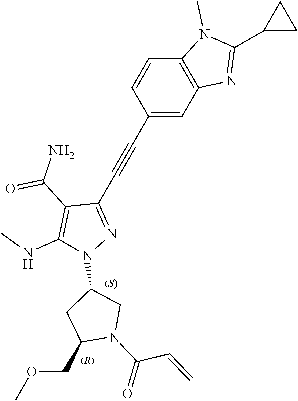 | 3-[2-(2-cyclopropyl-1-methyl-1,3- benzodiazol-5-yl)ethynyl]-1-[(3S,5R)- 5-(methoxymethyl)-1-(prop-2- enoyl)pyrrolidin-3-yl]-5- (methylamino)pyrazole-4-carboxamide |
| 44 | 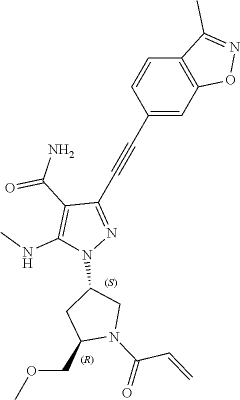 | 1-[(3S,5R)-5-(methoxymethyl)-1- (prop-2-enoyl)pyrrolidin-3-yl]-3-[2-(3- methyl-1,2-benzoxazol-6-yl)ethynyl]- 5-(methylamino)pyrazole-4- carboxamide |
| 45 | 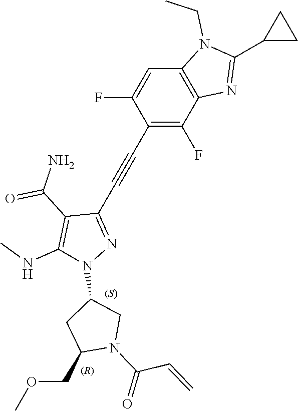 | 3-[2-(2-cyclopropyl-1-ethyl-4,6- difluoro-1,3-benzodiazol-5-yl) ethynyl]-1-[(3S,5R)-5- (methoxymethyl)-1-(prop-2-enoyl) pyrrolidin-3-yl]-5-(methylamino) pyrazole-4-carboxamide |
| 46 | 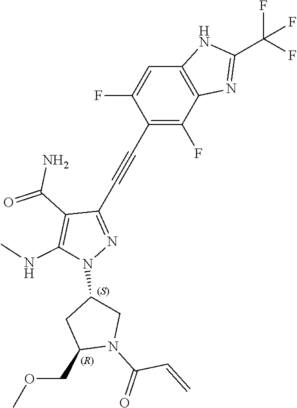 | 3-[2-[4,6-difluoro-2-(trifluoromethyl)- 1H-1,3-benzodiazol-5-yl]ethynyl]-1- [(3S,5R)-5-(methoxymethyl)-1-(prop- 2-enoyl)pyrrolidin-3-yl]-5- (methylamino)pyrazole-4-carboxamide |
| 47 | 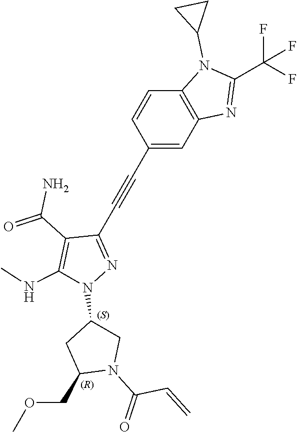 | 3-[2-[1-(difluoromethyl)-4,6-difluoro- 2-methyl-1,3-benzodiazol-5- yl]ethynyl]-1-[(3S,5R)-5- (methoxymethyl)-1-(prop-2- enoyl)pyrrolidin-3-yl]-5- (methylamino)pyrazole-4-carboxamide |
| 48 | 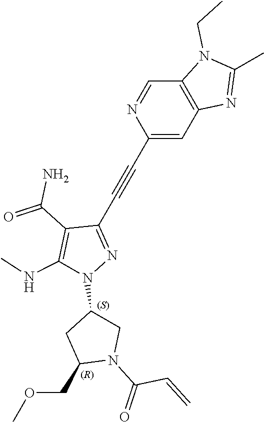 | 3-(2-[3-Ethyl-2-methylimidazo[4,5- c]pyridin-6-yl]ethynyl)-1-[(3S,5R)-5- (methoxymethyl)-1-(prop-2- enoyl)pyrrolidin-3-yl]-5- (methylamino)pyrazole-4-carboxamide |
| 49 | 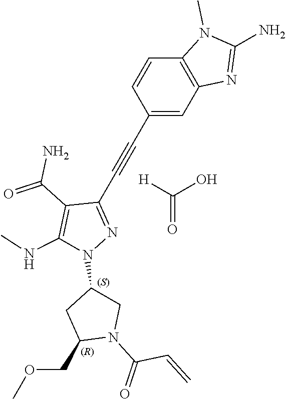 | 3-[2-(2-Amino-1-methyl-1,3- benzodiazol-5-yl)ethynyl]-1-[(3S,5R)- 5-(methoxymethyl)-1-(prop-2- enoyl)pyrrolidin-3-yl]-5- (methylamino)pyrazole-4- carboxamide; formic acid |
| 50 | 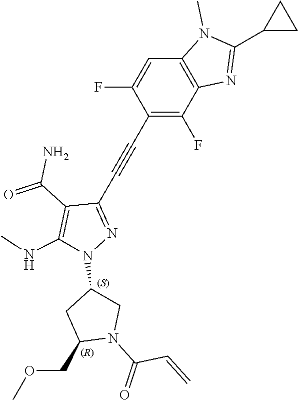 | 3-[2-(2-Cyclopropyl-4,6-difluoro-1- methyl-1,3-benzodiazol-5-yl)ethynyl]- 1-[(3S,5R)-5-(methoxymethyl)-1- (prop-2-enoyl)pyrrolidin-3-yl]-5- (methylamino)pyrazole-4-carboxamide |
| 51 | 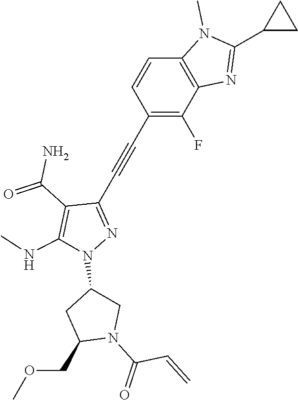 [SMILES: C=CC(=O)N1C[C@@H](n2nc(C#Cc3ccc4c(nc(C5CC5)n4C)c3F)c(C(N)=O)c2NC)C[C@@H]1COC] | 1-((3S,5R)-1-Acryloyl-5- (methoxymethyl)pyrrolidin-3-yl)-3- ((2-cyclopropyl-4-fluoro-1-methyl-1H- benzo[d]imidazol-5-yl)ethynyl)-5- (methylamino)-1H-pyrazole-4- carboxamide |
| 52 | 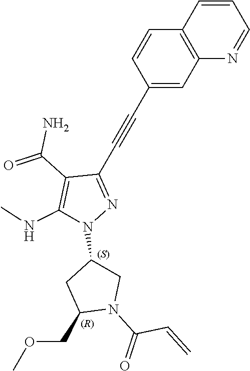 | 1-[(3S,5R)-5-(Methoxymethyl)-1- (prop-2-enoyl)pyrrolidin-3-yl]-5- (methylamino)-3-[2-(quinolin-7-yl) ethynyl]pyrazole-4-carboxamide |
| 53 | 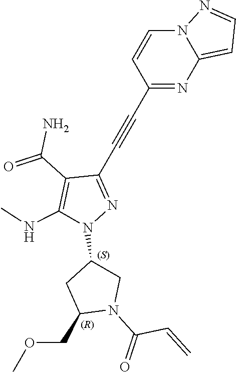 | 1-[(3S,5R)-5-(Methoxymethyl)-1- (prop-2-enoyl)pyrrolidin-3-yl]-5- (methylamino)-3-(2-[pyrazolo-[1,5- a]pyrimidin-5-yl]ethynyl)pyrazole-4- carboxamide |
| 54 | 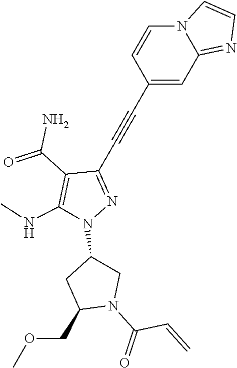 [SMILES: C=CC(=O)N1C[C@@H](n2nc(C#Cc3ccn4ccnc4c3)c(C(N)=O)c2NC)C[C@@H]1COC] | 1-((3S,5R)-1-Acryloyl-5- (methoxymethyl)pyrrolidin-3-yl)-3- (imidazo[1,2-a]pyridin-7-ylethynyl)-5- (methylamino)-1H-pyrazole-4- carboxamide |
| 55 | 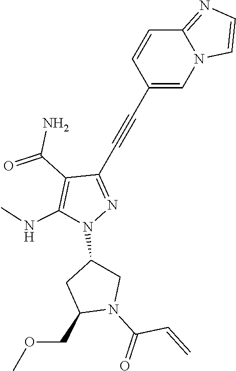 [SMILES: C=CC(=O)N1C[C@@H](n2nc(C#Cc3ccc4nccn4c3)c(C(N)=O)c2NC)C[C@@H]1COC] | 1-((3S,5R)-1-Acryloyl-5- (methoxymethyl)pyrrolidin-3-yl)-3- (imidazo[1,2-a]pyridin-6-ylethynyl)-5- (methylamino)-1H-pyrazole-4- carboxamide |
| 56 | 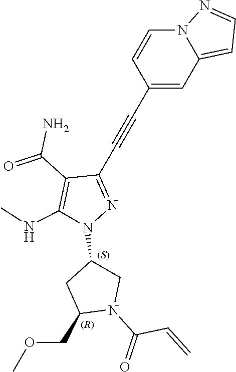 | 1-[(3S,5R)-5-(Methoxymethyl)-1- (prop-2-enoyl)pyrrolidin-3-yl]-5- (methylamino)-3-(2-[pyrazolo[1,5- a]pyridin-5-yl]ethynyl)pyrazole-4- carboxamide |
| 57 | 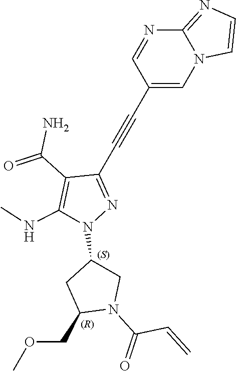 | 3-(2-[Imidazo[1,2-a]pyrimidin-6- yl]ethynyl)-1-[(3S,5R)-5- (methoxymethyl)-1-(prop-2- enoyl)pyrrolidin-3-yl]-5- (methylamino)pyrazole-4-carboxamide |
| 58 | 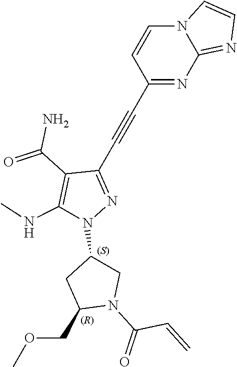 | 3-(2-[Imidazo[1,2-a]pyrimidin-7- yl]ethynyl)-1-[(3S,5R)-5- (methoxymethyl)-1-(prop-2- enoyl)pyrrolidin-3-yl]-5- (methylamino)pyrazole-4-carboxamide |
| 59 | 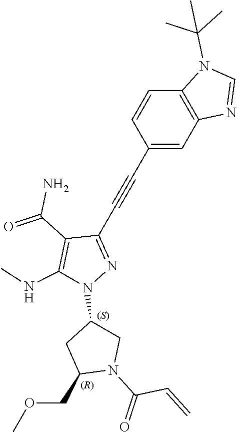 | 3-[2-(1-Tert-butyl-1,3-benzodiazol-5- yl)ethynyl]-1-[(3S,5R)-5- (methoxymethyl)-1-(prop-2- enoyl)pyrrolidin-3-yl]-5- (methylamino)pyrazole-4-carboxamide |
| 60 | 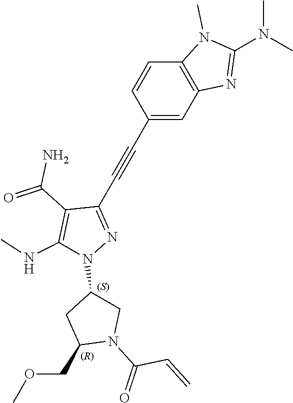 | 3-[2-[2-(Dimethylamino)-1-methyl- 1,3-benzodiazol-5-yl]ethynyl]-1- [(3S,5R)-5-(methoxymethyl)-1-(prop- 2-enoyl)pyrrolidin-3-yl]-5- (methylamino)pyrazole-4-carboxamide |
| 61 | 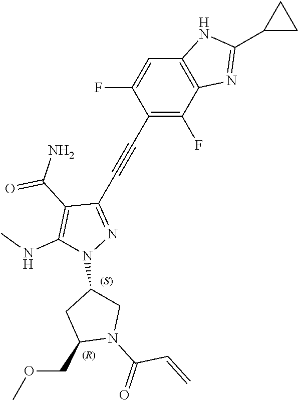 | 3-[2-(2-Cyclopropyl-4,6-difluoro-1H- 1,3-benzodiazol-5-yl)ethynyl]-1- [(3S,5R)-5-(methoxymethyl)-1-(prop- 2-enoyl)pyrrolidin-3-yl]-5- (methylamino)pyrazole-4-carboxamide |
| 62 | 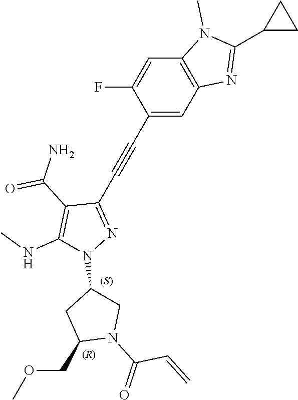 | 3-[2-(2-Cyclopropyl-6-fluoro-1- methyl-1,3-benzodiazol-5-yl)ethynyl]- 1-[(3S,5R)-5-(methoxymethyl)-1- (prop-2-enoyl)pyrrolidin-3-yl]-5- (methylamino)pyrazole-4-carboxamide |
| 63 | 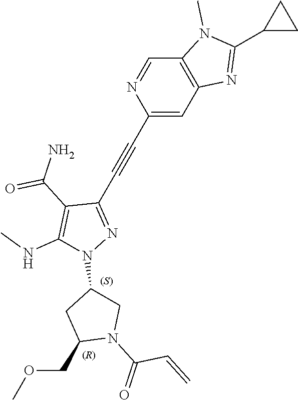 | 3-(2-[2-Cyclopropyl-3- methylimidazo[4,5-c]pyridin-6- yl]ethynyl)-1-[(3S,5R)-5- (methoxymethyl)-1-(prop-2- enoyl)pyrrolidin-3-yl]-5- (methylamino)pyrazole-4-carboxamide |
| 64 | 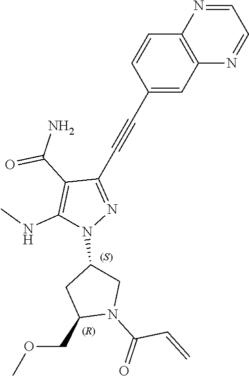 | 1-[(3S,5R)-5-(Methoxymethyl)-1- (prop-2-enoyl)pyrrolidin-3-yl]-5- (methylamino)-3-[2-(quinoxalin-6-yl) ethynyl]pyrazole-4-carboxamide |
| 65 | 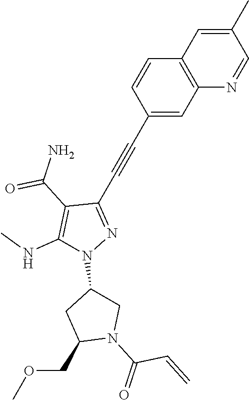 | 1-((3S,5R)-1-Acryloyl-5- (methoxymethyl)pyrrolidin-3-yl)-5- (methylamino)-3-((3-methyquinolin- 7-yl)ethynyl)-1H-pyrazole-4- carboxamide |
| 66 | 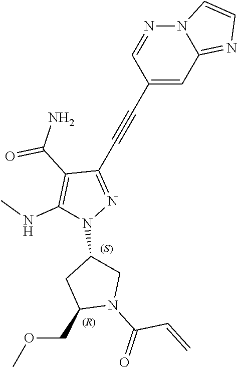 | 3-(2-[Imidazo[1,2-b]pyridazin-7- yl]ethynyl)-1-[(3S,5R)-5- (methoxymethyl)-1-(prop-2- enoyl)pyrrolidin-3-yl]-5- (methylamino)pyrazole-4-carboxamide |
| 67 | 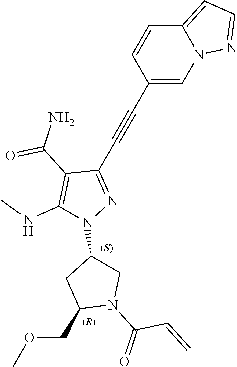 | 1-[(3S,5R)-5-(Methoxymethyl)-1- (prop-2-enoyl)pyrrolidin-3-yl]-5- (methylamino)-3-(2-[pyrazolo[1,5- a]pyridin-6-yl]ethynyl)pyrazole-4- carboxamide |
| 68 | 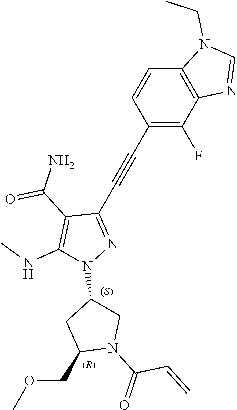 | 1-((3S,5R)-1-Acryloyl-5- (methoxymethyl)pyrrolidin-3-yl)-3- ((1-ethyl-4-fluoro-1H- benzo[d]imidazol-5-yl)ethynyl)-5- (methylamino)-1H-pyrazole-4- carboxamide |
| 69 | 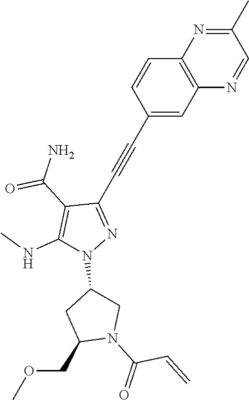 | 1-((3S,5R)-1-Acryloyl-5- (methoxymethyl)pyrrolidin-3-yl)-5- (methylamino)-3-((2- methylquinoxalin-6-yl)ethynyl)-1H- pyrazole-4-carboxamide |
| 70 | 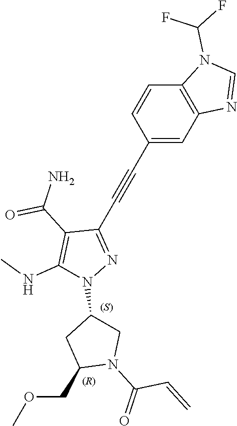 | 3-[2-[1-(Difluoromethyl)-1,3- benzodiazol-5-yl]ethynyl]-1-[(3S,5R)- 5-(methoxymethyl)-1-(prop-2- enoyl)pyrrolidin-3-yl]-5- (methylamino)pyrazole-4-carboxamide |
| 71 | 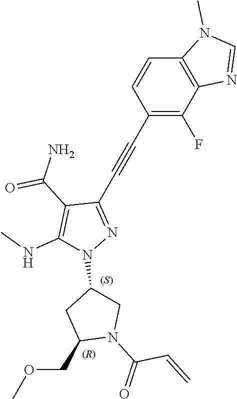 | 1-((3S,5R)-1-Acryloyl-5- (methoxymethyl)pyrrolidin-3-yl)-3- ((4-fluoro-1-methyl-1H- benzo[d]imidazol-5-yl)ethynyl)-5- (methylamino)-1H-pyrazole-4- carboxamide |
| 72 | 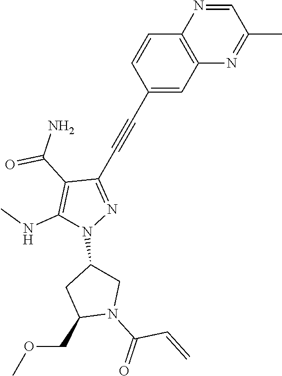 | 1-((3S,5R)-1-Acryloyl-5- (methoxymethyl)pyrrolidin-3-yl)-5- (methylamino)-3-((3- methylquinoxalin-6-yl)ethynyl)-1H- pyrazole-4-carboxamide |
| 73 | 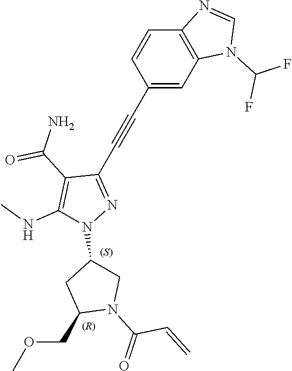 | 3-[2-[3-(Difluoromethyl)-1,3- benzodiazol-5-yl]ethynyl]-1-[(3S,5R)- 5-(methoxymethyl)-1-(prop-2- enoyl)pyrrolidin-3-yl]-5- (methylamino)pyrazole-4-carboxamide |
| 74 | 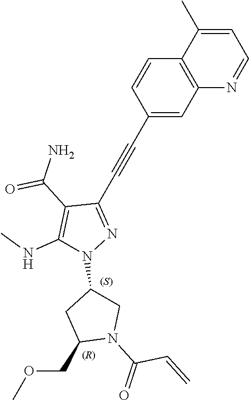 | 1-[(3S,5R)-5-(Methoxymethyl)-1- (prop-2-enoyl)pyrrolidin-3-yl]-5- (methylamino)-3-[2-(4- methylquinolin-7-yl)ethynyl]pyrazole- 4-carboxamide |
| 75 | 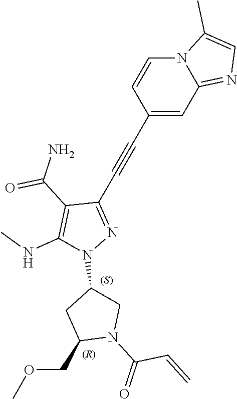 | 1-[(3S,5R)-5-(Methoxymethyl)-1- (prop-2-enoyl)pyrrolidin-3-yl]-5- (methylamino)-3-(2-[3- methylimidazo[1,2-a]pyridin-7-yl] ethynyl)pyrazole-4-carboxamide |
| 76 | 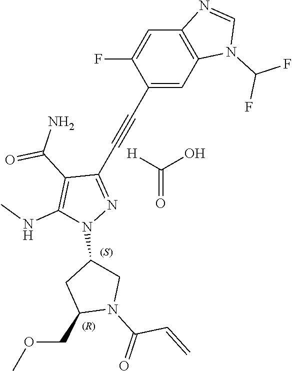 | 3-[2-[3-(Difluoromethyl)-6-fluoro-1,3- benzodiazol-5-yl]ethynyl]-1-[(3S,5R)- 5-(methoxymethyl)-1-(prop-2- enoyl)pyrrolidin-3-yl]-5- (methylamino)pyrazole-4- carboxamide; formic acid |
| 77 | 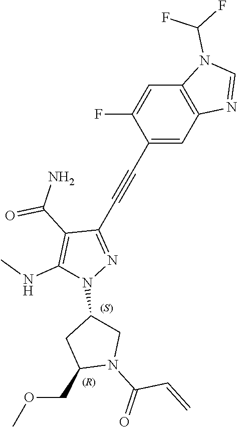 | 3-[2-[1-(Difluoromethyl)-6-fluoro-1,3- benzodiazol-5-yl]ethynyl]-1-[(3S,5R)- 5-(methoxymethyl)-1-(prop-2- enoyl)pyrrolidin-3-yl]-5- (methylamino)pyrazole-4-carboxamide |
| 78 | 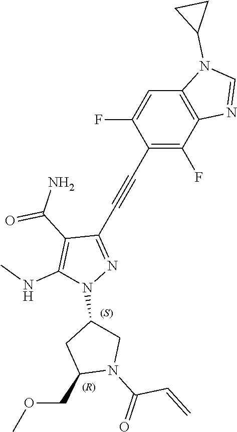 | 3-[2-(1-Cyclopropyl-4,6-difluoro-1,3- benzodiazol-5-yl)ethynyl]-1-[(3S,5R)- 5-(methoxymethyl)-1-(prop-2- enoyl)pyrrolidin-3-yl]-5- (methylamino)pyrazole-4-carboxamide |
| 79 | 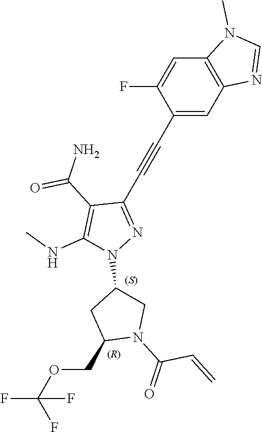 | 3-[2-(6-Fluoro-1-methyl-1,3- benzodiazol-5-yl)ethynyl]-5- (methylamino)-1-[(3S,5R)-1-(prop-2- enoyl)-5- [(trifluoromethoxy)methyl]pyrrolidin- 3-yl]pyrazole-4-carboxamide |
| 80 | 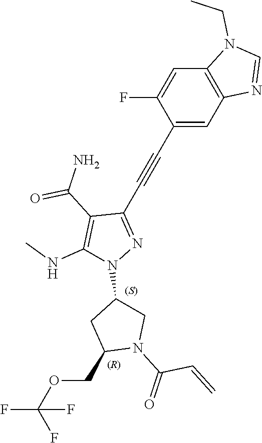 | 3-[2-(1-Ethyl-6-fluoro-1,3- benzodiazol-5-yl)ethynyl]-5- (methylamino)-1-[(3S,5R)-1-(prop-2- enoyl)-5- [(trifluoromethoxy)methyl]pyrrolidin- 3-yl]pyrazole-4-carboxamide |
| 81 | 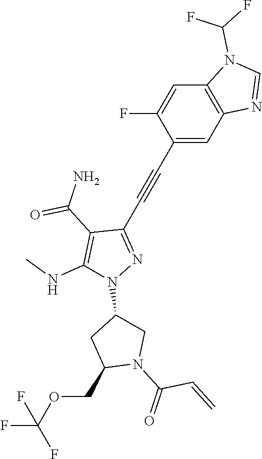 [SMILES: C=CC(=O)N1C[C@@H](n2nc(C#Cc3cc4ncn(C(F)F)c4cc3F)c(C(N)=O)c2NC)C[C@@H]1COC(F)(F)F] | 3-[2-[1-(Difluoromethyl)-6-fluoro-1,3- benzodiazol-5-yl]ethynyl]-5- (methylamino)-1-[(3S,5R)-1-(prop-2- enoyl)-5- [(trifluoromethoxy)methyl]pyrrolidin- 3-yl]pyrazole-4-carboxamide |
| 82 | 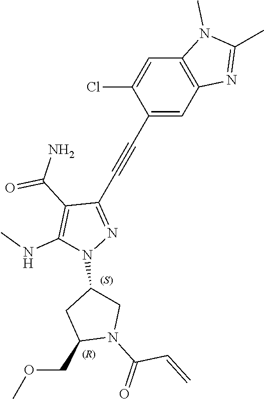 | 3-[2-(6-Chloro-1,2-dimethyl-1,3- benzodiazol-5-yl)ethynyl]-1-[(3S,5R)- 5-(methoxymethyl)-1-(prop-2- enoyl)pyrrolidin-3-yl]-5- (methylamino)pyrazole-4-carboxamide |
| 83 | 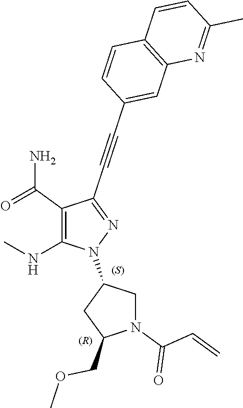 | 1-[(3S,5R)-5-(Methoxymethyl)-1- (prop-2-enoyl)pyrrolidin-3-yl]-5- (methylamino)-3-[2-(2- methylquinolin-7-yl)ethynyl]pyrazole- 4-carboxamide |
| 84 | 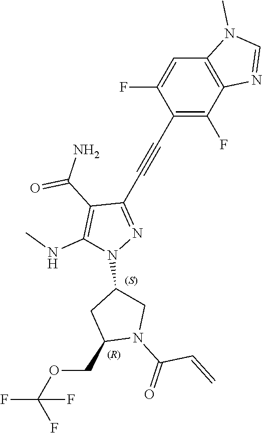 | 3-[2-(4,6-Difluoro-1-methyl-1,3- benzodiazol-5-yl)ethynyl]-5- (methylamino)-1-[(3S,5R)-1-(prop-2- enoyl)-5- [(trifluoromethoxy)methyl]pyrrolidin- 3-yl]pyrazole-4-carboxamide |
| 85 | 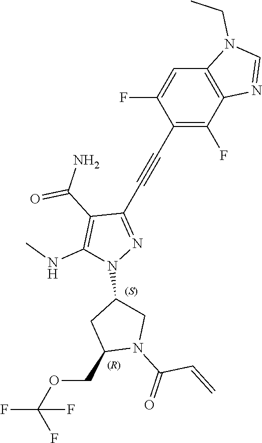 | 3-[2-(1-Ethyl-4,6-difluoro-1,3- benzodiazol-5-yl)ethynyl]-5- (methylamino)-1-[(3S,5R)-1-(prop-2- enoyl)-5- [(trifluoromethoxy)methyl]pyrrolidin- 3-yl]pyrazole-4-carboxamide |
| 86 | 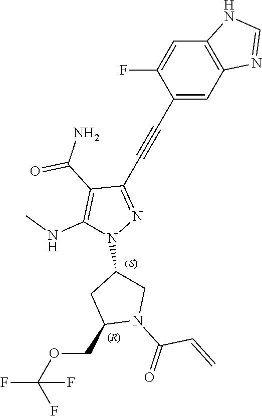 | 3-[2-(6-Fluoro-1H-1,3-benzodiazol-5- yl)ethynyl]-5-(methylamino)-1- [(3S,5R)-1-(prop-2-enoyl)-5- [(trifluoromethoxy)methyl]pyrrolidin- 3-yl]pyrazole-4-carboxamide |
| 87 | 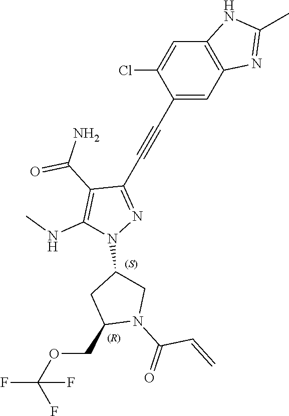 | 3-[2-(6-Chloro-2-methyl-1H-1,3- benzodiazol-5-yl)ethynyl]-5- (methylamino)-1-[(3S,5R)-1-(prop-2- enoyl)-5- [(trifluoromethoxy)methyl]pyrrolidin- 3-yl]pyrazole-4-carboxamide |
| 88 | 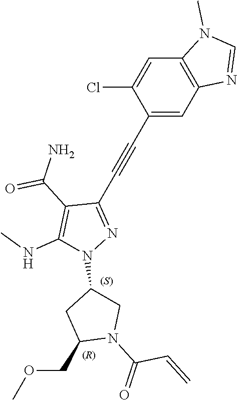 | 3-[2-(6-Chloro-1-methyl-1,3- benzodiazol-5-yl)ethynyl]-1-[(3S,5R)- 5-(methoxymethyl)-1-(prop-2- enoyl)pyrrolidin-3-yl]-5- (methylamino)pyrazole-4-carboxamide |
| 89 | 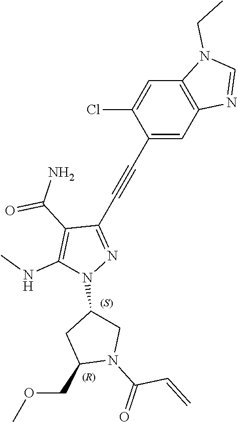 | 3-[2-(6-Chloro-1-ethyl-1,3- benzodiazol-5-yl)ethynyl]-1-[(3S,5R)- 5-(methoxymethyl)-1-(prop-2- enoyl)pyrrolidin-3-yl]-5- (methylamino)pyrazole-4-carboxamide |
| 90 | 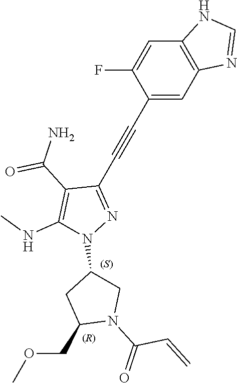 | 3-[2-(6-Fluoro-1H-1,3-benzodiazol-5- yl)ethynyl]-1-[(3S,5R)-5- (methoxymethyl)-1-(prop-2- enoyl)pyrrolidin-3-yl]-5- (methylamino)pyrazole-4-carboxamide |
| 91 | 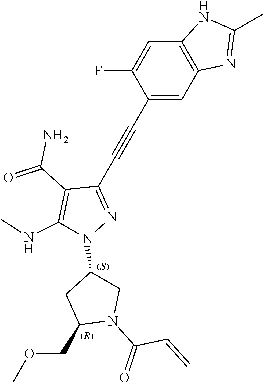 | 3-[2-(6-Fluoro-2-methyl-1H-1,3- benzodiazol-5-yl)ethynyl]-1-[(3S,5R)- 5-(methoxymethyl)-1-(prop-2- enoyl)pyrrolidin-3-yl]-5- (methylamino)pyrazole-4-carboxamide |
| 92 | 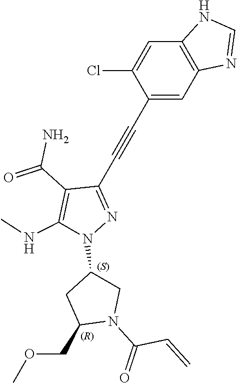 | 3-[2-(6-Chloro-1H-1,3-benzodiazol-5- yl)ethynyl]-1-[(3S,5R)-5- (methoxymethyl)-1-(prop-2- enoyl)pyrrolidin-3-yl]-5- (methylamino)pyrazole-4-carboxamide |
| 93 | 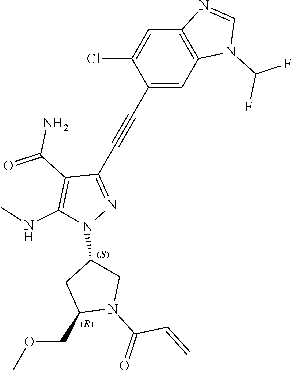 | 3-[2-[6-Chloro-3-(difluoromethyl)-1,3- benzodiazol-5-yl]ethynyl]-1-[(3S,5R)- 5-(methoxymethyl)-1-(prop-2- enoyl)pyrrolidin-3-yl]-5- (methylamino)pyrazole-4-carboxamide |
| 94 | 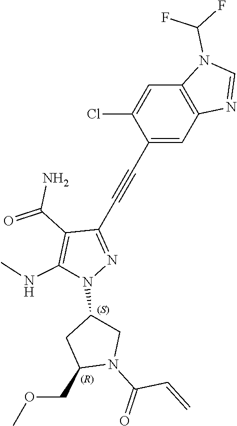 | 3-[2-[6-Chloro-1-(difluoromethyl)-1,3- benzodiazol-5-yl]ethynyl]-1-[(3S,5R)- 5-(methoxymethyl)-1-(prop-2- enoyl)pyrrolidin-3-yl]-5- (methylamino)pyrazole-4-carboxamide |
| 95 | 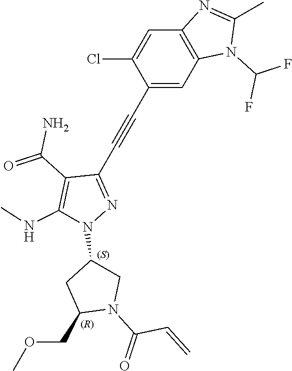 | 3-[2-[6-Chloro-3-(difluoromethyl)-2- methyl-1,3-benzodiazol-5-yl]ethynyl]- 1-[(3S,5R)-5-(methoxymethyl)-1- (prop-2-enoyl)pyrrolidin-3-yl]-5- (methylamino)pyrazole-4-carboxamide |
| 96 | 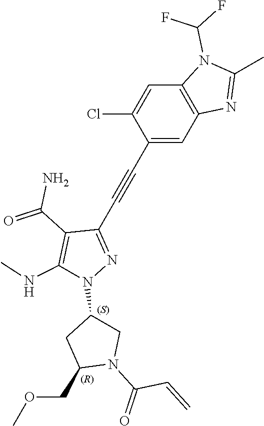 | 3-[2-[6-Chloro-1-(difluoromethyl)-2- methyl-1,3-benzodiazol-5-yl]ethynyl]- 1-[(3S,5R)-5-(methoxymethyl)-1- (prop-2-enoyl)pyrrolidin-3-yl]-5- (methylamino)pyrazole-4-carboxamide |
| 97 | 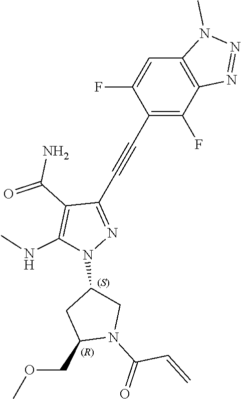 | 3-[2-(4,6-Difluoro-1-methyl-1,2,3- benzotriazol-5-yl)ethynyl]-1-[(3S,5R)- 5-(methoxymethyl)-1-(prop-2- enoyl)pyrrolidin-3-yl]-5- (methylamino)pyrazole-4-carboxamide |
| 98 | 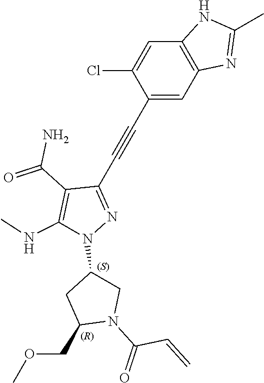 | 3-[2-(6-Chloro-2-methyl-1H-1,3- benzodiazol-5-yl)ethynyl]-1-[(3S,5R)- 5-(methoxymethyl)-1-(prop-2- enoyl)pyrrolidin-3-yl]-5- (methylamino)pyrazole-4-carboxamid |
| 99 | 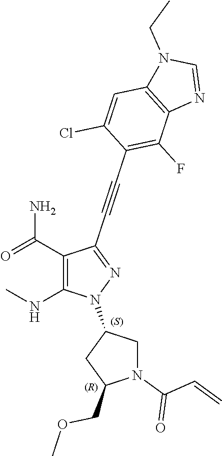 | 3-[2-(6-Chloro-1-ethyl-4-fluoro-1,3- benzodiazol-5-yl)ethynyl]-1-[(3S,5R)- 5-(methoxymethyl)-1-(prop-2- enoyl)pyrrolidin-3-yl]-5- (methylamino)pyrazole-4-carboxamide |
| 100 | 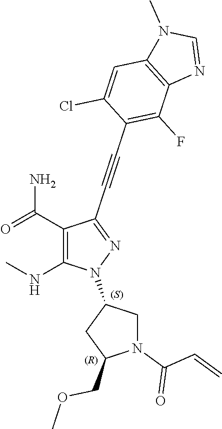 | 3-[2-(6-Chloro-4-fluoro-1-methyl-1,3- benzodiazol-5-yl)ethynyl]-1-[(3S,5R)- 5-(methoxymethyl)-1-(prop-2- enoyl)pyrrolidin-3-yl]-5- (methylamino)pyrazole-4-carboxamide |
| 101 | 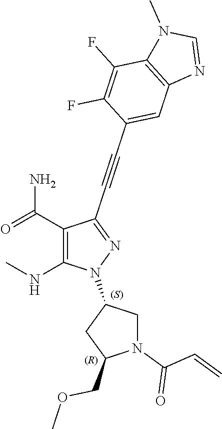 | 3-[2-(6,7-Difluoro-1-methyl-1,3- benzodiazol-5-yl)ethynyl]-1-[(3S,5R)- 5-(methoxymethyl)-1-(prop-2- enoyl)pyrrolidin-3-yl]-5- (methylamino)pyrazole-4-carboxamide |
| 102 | 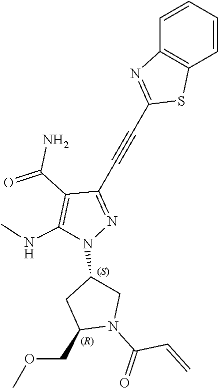 | 3-[2-(1,3-Benzothiazol-2-yl)ethynyl]- 1-[(3S,5R)-5-(methoxymethyl)-1- (prop-2-enoyl)pyrrolidin-3-yl]-5- (methylamino)pyrazole-4-carboxamide |
| 103 | 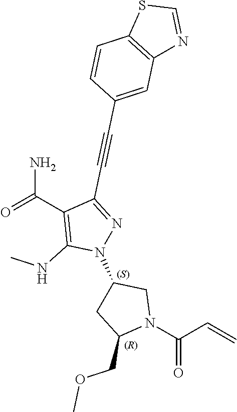 | 3-[2-(1,3-Benzothiazol-2-yl)ethynyl]- 1-[(3S,5R)-5-(methoxymethyl)-1- (prop-2-enoyl)pyrrolidin-3-yl]-5- (methylamino)pyrazole-4-carboxamide |
| 104 | 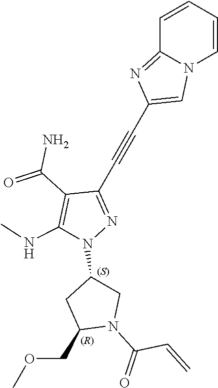 | 3-(2-[Imidazol[1,2-a]pyridin-2- yl]ethynyl)-1-[(3S,5R)-5- (methoxymethyl)-1-(prop-2- enoyl)pyrrolidin-3-yl]-5- (methylamino)pyrazole-4-carboxamide |
| 105 | 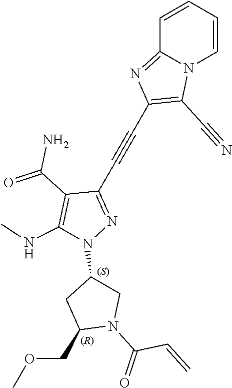 | 1-((3S,5R)-1-Acryloyl-5- (methoxymethyl)pyrrolidin-3-yl)-3- ((3-cyanoimidazo[1,2-a]pyridin-2- yl)ethynyl)-5-(methylamino)-1H- pyrazole-4-carboxamide |
| 106 | 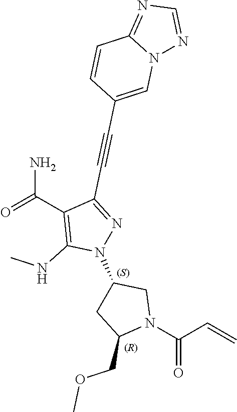 | 1-[(3S,5R)-5-(Methoxymethyl)-1- (prop-2-enoyl)pyrrolidin-3-yl]-5- (methylamino)-3-(2- [[1,2,4]triazolo[1,5-a]pyridin-6- yl]ethynyl)pyrazole-4-carboxamide |
| 107 | 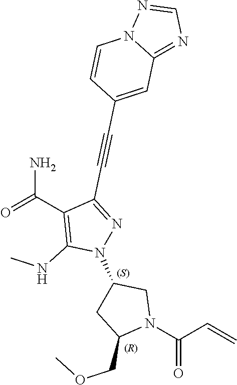 | 1-[(3S,5R)-5-(Methoxymethyl)-1- (prop-2-enoyl)pyrroldin-3-yl]-5- (methylamino)-3-(2- [[1,2,4]triazolo[1,5-a]pyridin-7- yl]ethynyl)pyrazole-4-carboxamide |
| 108 | 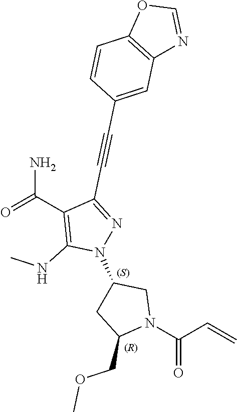 | 3-[2-(1,3-Benzoxazol-5-yl)ethynyl]-1- [(3S,5R)-5-(methoxymethyl)-1-(prop- 2-enoyl)pyrrolidin-3-yl]-5- (methylamino)pyrazole-4-carboxamide |
| 109 | 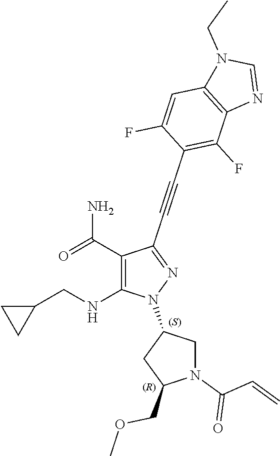 | 5-[(Cyclopropylmethyl)amino]-3-[2- (1-ethyl-4,6-difluoro-1,3-benzodiazol- 5-yl)ethynyl]-1-[(3S,5R)-5- (methoxymethyl)-1-(prop-2- enoyl)pyrrolidin-3-yl]pyrazole-4- carboxamide |
| 110 | 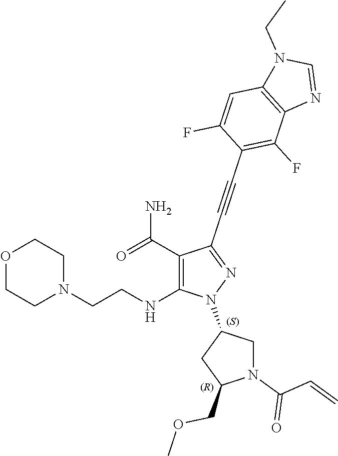 | 3-[2-[1-(Difluoromethyl)-4,6-difluoro- 2-methyl-1,3-benzodiazol-5- yl]ethynyl]-1-[(3S,5R)-5- (methoxymethyl)-1-(prop-2- enoyl)pyrrolidin-3-yl]-5- (methylamino)pyrazole-4-carboxamide |
| 111 | 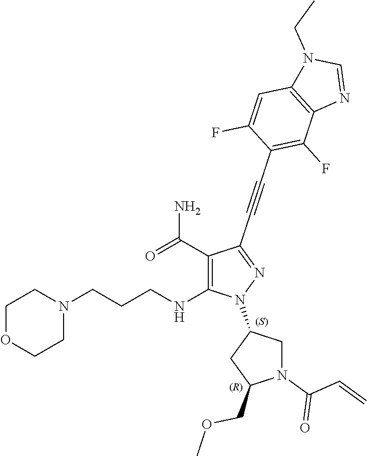 | 3-[2-(1-Ethyl-4,6-difluoro-1,3- benzodiazol-5-yl)ethynyl]-1-[(3S,5R)- 5-(methoxymethyl)-1-(prop-2- enoyl)pyrrolidin-3-yl]-5-[[3- (morpholin-4- yl)propyl]amino]pyrazole-4- carboxamide |
| 112 | 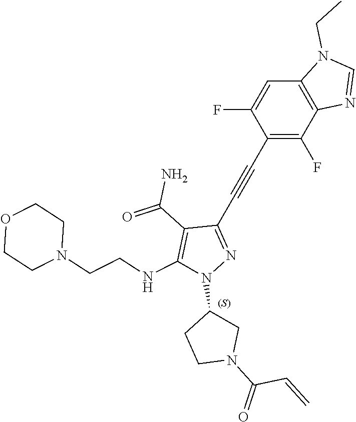 | 3-[2-(1-Ethyl-4,6-difluoro-1,3- benzodiazol-5-yl)ethynyl]-5-[[2- (morpholin-4-yl)ethyl]amino]-1-[(3S)- 1-(prop-2-enoyl)pyrrolidin-3- yl]pyrazole-4-carboxamid |
| 113 | 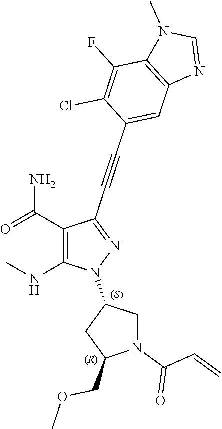 | 3-[2-(6-Chloro-7-fluoro-1-methyl-1,3- benzodiazol-5-yl)ethynyl]-1-[(3S,5R)- 5-(methoxymethyl)-1-(prop-2- enoyl)pyrrolidin-3-yl]-5- (methylamino)pyrazole-4-carboxamide |
| 114 | 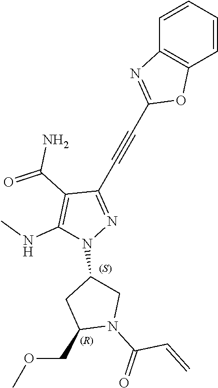 | 3-[2-(1,3-Benzoxazol-2-yl)ethynyl]-1- [(3S,5R)-5-(methoxymethyl)-1-(prop- 2-enoyl)pyrrolidin-3-yl]-5- (methylamino)pyrazole-4-carboxamide |
| 115 | 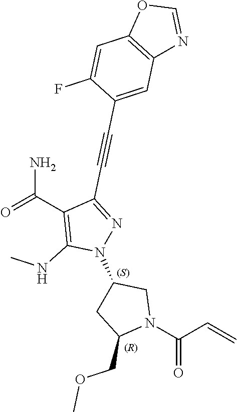 | 3-[2-(6-Fluoro-1,3-benzoxazol-5- yl)ethynyl]-1-[(3S,5R)-5- (methoxymethyl)-1-(prop-2- enoyl)pyrrolidin-3-yl]-5- (methylamino)pyrazole-4-carboxamide |
| 116 | 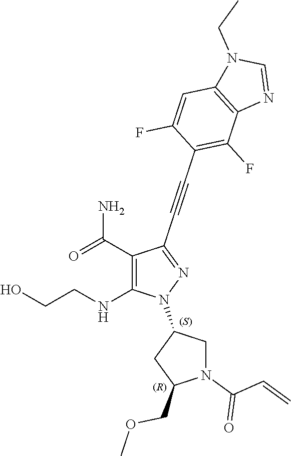 | 3-[2-(1-Ethyl-4,6-difluoro-1,3- benzodiazol-5-yl)ethynyl]-5-[(2- hydroxyethyl)amino]-1-[(3S,5R)-5- (methoxymethyl)-1-(prop-2- enoyl)pyrrolidin-3-yl]pyrazole-4- carboxamide |
| 117 | 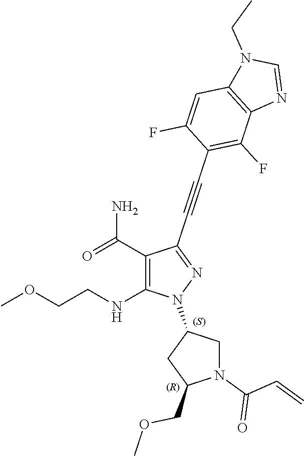 | 3-[2-(1-Ethyl-4,6-difluoro-1,3- benzodiazol-5-yl)ethynyl]-5-[(2- methoxyethyl)amino]-1-[(3S,5R)-5- (methoxymethyl)-1-(prop-2- enoyl)pyrrolidin-3-yl]pyrazole-4- carboxamide |
| 118 | 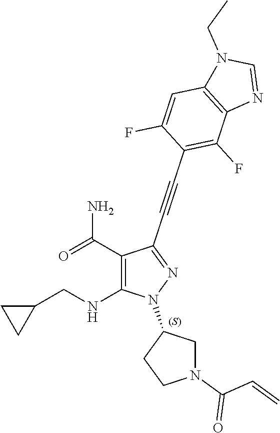 | 5-[(Cyclopropylmethyl)amino]-3-[2- (1-ethyl-4,6-difluoro-1,3-benzodiazol- 5-yl)ethynyl]-1-[(3S)-1-(prop-2- enoyl)pyrrolidin-3-yl]pyrazole-4- carboxamide |
| 119 | 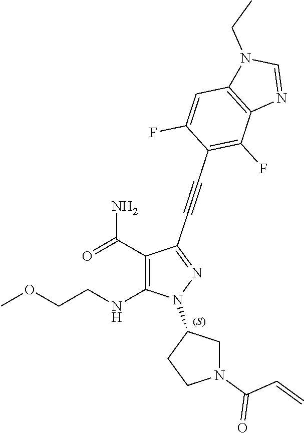 | 3-[2-(1-Ethyl-4,6-difluoro-1,3- benzodiazol-5-yl)ethynyl]-5-[(2- methoxyethyl)amino]-1-[(3S)-1-(prop- 2-enoyl)pyrrolidin-3-yl]pyrazole-4- carboxamide |
| 120 | 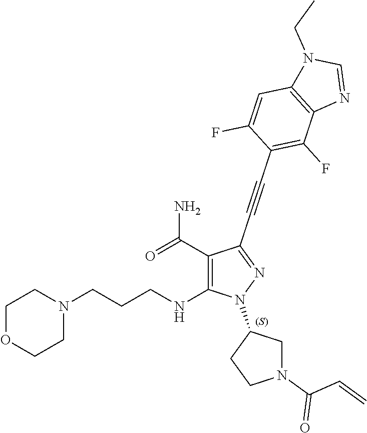 | 3-[2-(1-Ethyl-4,6-difluoro-1,3- benzodiazol-5-yl)ethynyl]-5-[[3- (morpholin-4-yl)propyl]amino]-1- [(3S)-1-(prop-2-enoyl)pyrrolidin-3- yl]pyrazole-4-carboxamide |
| 121 | 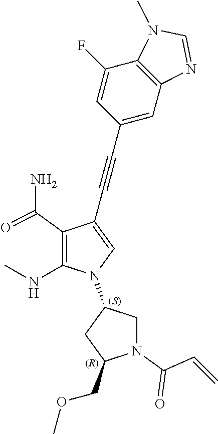 | 1-((3S,5R)-1-Acryloyl-5- (methoxymethyl)pyrrolidin-3-yl)-3- ((4-fluoro-1-methyl-1H- benzo[d]imidazol-5-yl)ethynyl)-5- (methylamino)-1H-pyrazole-4- carboxamide |
| 122 | 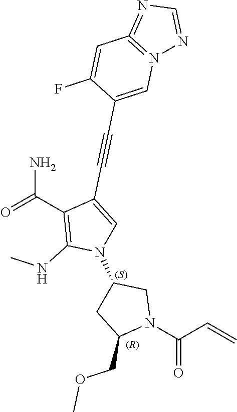 | 3-(2-[7-Fluoro-[1,2,4]triazolo[1,5- a]pyridin-6-yl]ethynyl)-1-[(3S,5R)-5- (methoxymethyl)-1-(prop-2- enoyl)pyrrolidin-3-yl]-5- (methylamino)pyrazole-4-carboxamide |
| 123 | 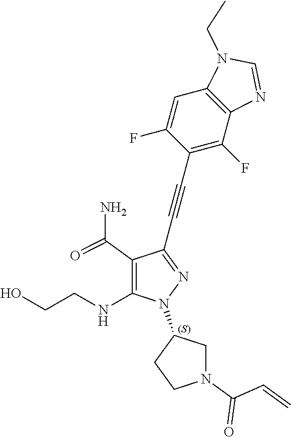 | 3-[2-(1-Ethyl-4,6-difluoro-1,3- benzodiazol-5-yl)ethynyl]-5-[(2- hydroxyethyl)amino]-1-[(3S)-1-(prop- 2-enoyl)pyrroldiin-3-yl]pyrazole-4- carboxamide |
| 124 | 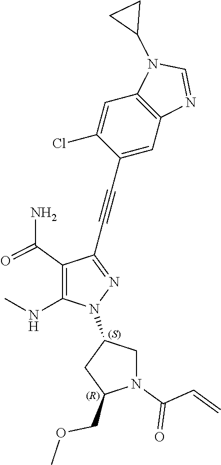 | 3-[2-(6-Chloro-1-cyclopropyl-1,3- benzodiazol-5-yl)ethynyl]-1-[(3S,5R)- 5-(methoxymethyl)-1-(prop-2- enoyl)pyrrolidin-3-yl]-5- (methylamino)pyrazole-4-carboxamide |
| 125 | 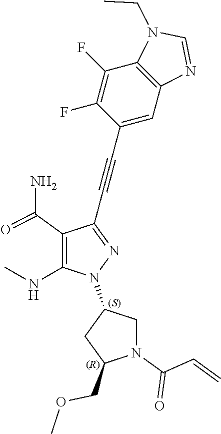 | 3-[2-(1-Ethyl-6,7-difluoro-1,3- benzodiazol-5-yl)ethynyl]-1-[(3S,5R)- 5-(methoxymethyl)-1-(prop-2- enoyl)pyrrolidin-3-yl]-5- (methylamino)pyrazole-4-carboxamide |
| 126 | 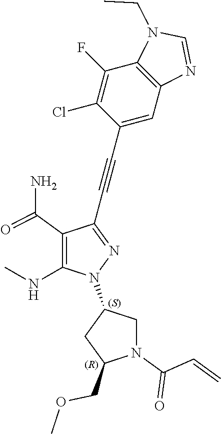 | 3-[2-(6-Chloro-1-ethyl-7-fluoro-1,3- benzodiazol-5-yl)ethynyl]-1-[(3S,5R)- 5-(methoxymethyl)-1-(prop-2- enoyl)pyrrolidin-3-yl]-5- (methylamino)pyrazole-4-carboxamide |
| 127 | 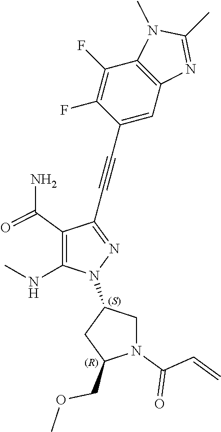 | 3-[2-(6,7-Difluoro-1,2-dimethyl-1,3- benzodiazol-5-yl)ethynyl]-1-[(3S,5R)- 5-(methoxymethyl)-1-(prop-2-enoyl) pyrrolidin-3-yl]-5-(methylamino) pyrazole-4-carboxamide |
| 128 | 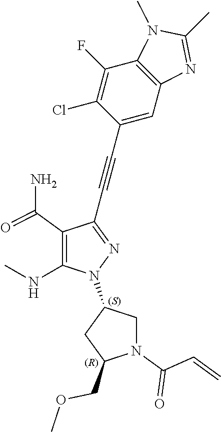 | 3-[2-(6-Chloro-7-fluoro-1,2-dimethyl- 1,3-benzodiazol-5-yl)ethynyl]-1- [(3S,5R)-5-(methoxymethyl)-1-(prop- 2-enoyl)pyrrolidin-3-yl]-5- (methylamino)pyrazole-4-carboxamide |
| 129 | 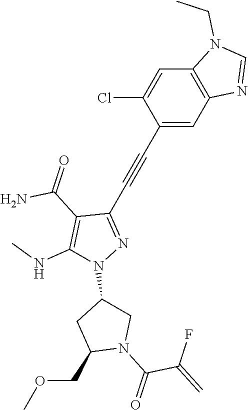 [SMILES: C=C(F)C(=O)N1C[C@@H](n2nc(C#Cc3cc4ncn(CC)c4cc3Cl)c(C(N)=O)c2NC)C[C@@H]1COC] | 3-[2-(6-Chloro-1-ethyl-1,3- benzodiazol-5-yl)ethynyl]-1-[(3S,5R)- 1-(2-fluoroprop-2-enoyl)-5- (methoxymethyl)pyrrolidin-3-yl]-5- (methylamino)pyrazole-4-carboxamide |
| 130 | 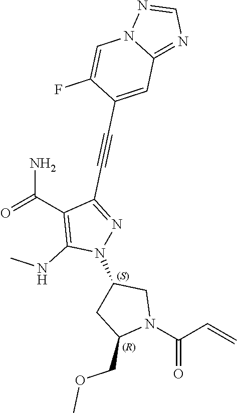 | 3-(2-[6-Fluoro-[1,2,4]triazolo[1,5- a]pyridin-7-yl]ethynyl)-1-[(3S,5R)-5- (methoxymethyl)-1-(prop-2- enoyl)pyrrolidin-3-yl]-5- (methylamino)pyrazole-4-carboxamide |
| 131 | 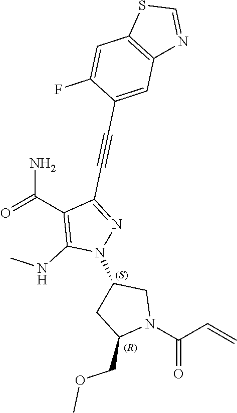 | 3-[2-(6-Fluoro-1,3-benzothiazol-5- yl)ethynyl]-1-[(3S,5R)-5- (methoxymethyl)-1-(prop-2- enoyl)pyrrolidin-3-yl]-5- (methylamino)pyrazole-4-carboxamide |
| 132 | 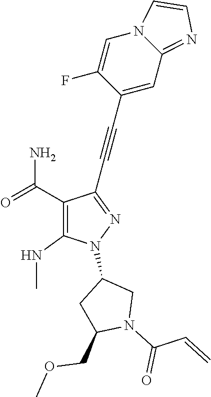 [SMILES: C=CC(=O)N1C[C@@H](n2nc(C#Cc3cc4nccn4cc3F)c(C(N)=O)c2NC)C[C@@H]1COC] | 1-((3S,5R)-1-Acryloyl-5- (methoxymethyl)pyrrolidin-3-yl)-3- ((6-fluoroimidazo[1,2-a]pyridin-7- yl)ethynyl)-5-(methylamino)-1H- pyrazole-4-carboxamide |
| 133 | 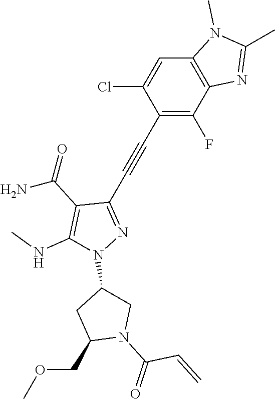 | 3-[2-(6-Chloro-4-fluoro-1,2-dimethyl- 1,3-benzodiazol-5-yl)ethynyl]-1- [(3S,5R)-5-(methoxymethyl)-1-(prop- 2-enoyl)pyrrolidin-3-yl]-5- (methylamino)pyrazole-4-carboxamide |
| 134 | 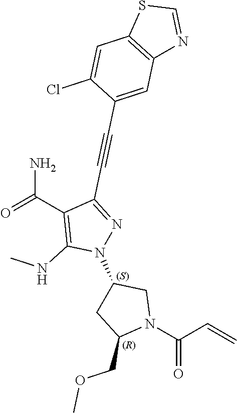 | 3-[2-(6-Chloro-1,3-benzothiazol-5- yl)ethynyl]-1-[(3S,5R)-5- (methoxymethyl)-1-(prop-2- enoyl)pyrrolidin-3-yl]-5- (methylamino)pyrazole-4-carboxamide |
| 135 | 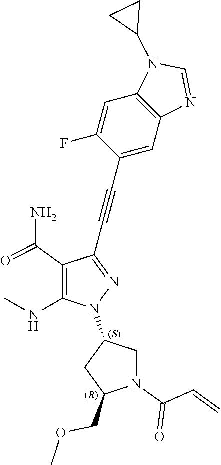 | 3-[2-(1-Cyclopropyl-6-fluoro-1,3- benzodiazol-5-yl)ethynyl]-1-[(3S,5R)- 5-(methoxymethyl)-1-(prop-2- enoyl)pyrrolidin-3-yl]-5- (methylamino)pyrazole-4-carboxamide |
| 136 | 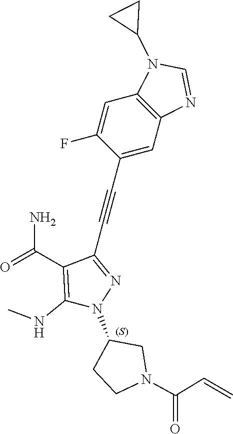 | 3-[2-(1-Cyclopropyl-6-fluoro-1,3- benzodiazol-5-yl)ethynyl]-5- (methylamino)-1-[(3S)-1-(prop-2- enoyl)pyrrolidin-3-yl]pyrazole-4- carboxamide |
| 137 | 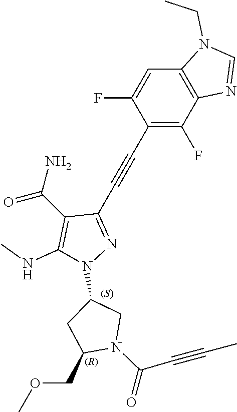 | 1-((3S,5R)-1-(but-2-ynoyl)-5- (methoxymethyl)pyrrolidin-3-yl)-3- ((1-ethyl-4,6-difluoro-1H- benzo[d]imidazol-5-yl)ethynyl)-5- (methylamino)-1H-pyrazole-4- carboxamide |
| 138 | 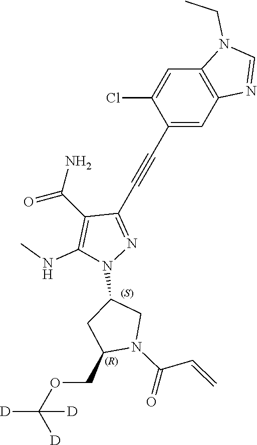 | 1-((3S,5R)-1-acryloyl-5-((methoxy- d3)methyl)pyrrolidin-3-yl)-3-((6- chloro-1-ethyl-1H-benzo[d]imidazol- 5-yl)ethynyl)-5-(methylamino)-1H- pyrazole-4-carboxamide |
| 139 | 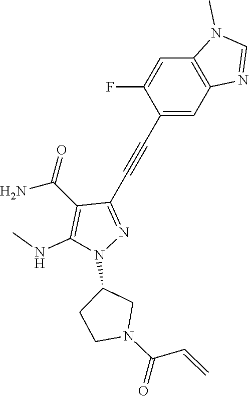 [SMILES: C=CC(=O)N1CC[C@H](n2nc(C#Cc3cc4ncn(C)c4cc3F)c(C(N)=O)c2NC)C1] | 3-[2-(6-fluoro-1-methyl-1,3- benzodiazol-5-yl)ethynyl]-5- (methylamino)-1-[(3S)-1-(prop-2- enoyl)pyrrolidin-3-yl]pyrazole-4- carboxamide |
| 140 | 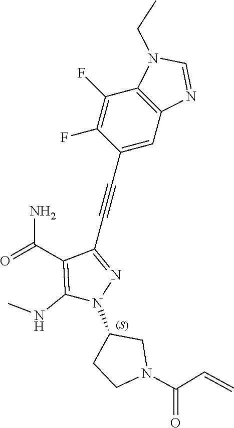 | 3-[2-(1-ethyl-6,7-difluoro-1,3- benzodiazol-5-yl)ethynyl]-5- (methylamino)-1-[(3S)-1-(prop-2- enoyl)pyrrolidin-3-yl]pyrazole-4- carboxamide |
| 141 | 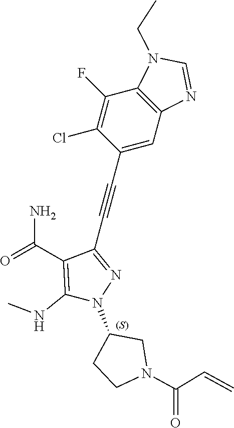 | 3-[2-(6-chloro-1-ethyl-7-fluoro-1,3- benzodiazol-5-yl)ethynyl]-5- (methylamino)-1-[(3S)-1-(prop-2- enoyl)pyrrolidin-3-yl]pyrazole-4- carboxamide |
| 142 | 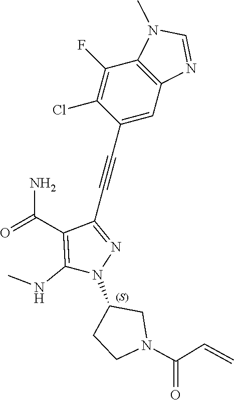 | 3-[2-(6-chloro-7-fluoro-1-methyl-1,3- benzodiazol-5-yl)ethynyl]-5- (methylamino)-1-[(3S)-1-(prop-2- enoyl)pyrrolidin-3-yl]pyrazole-4- carboxamide |
| 143 | 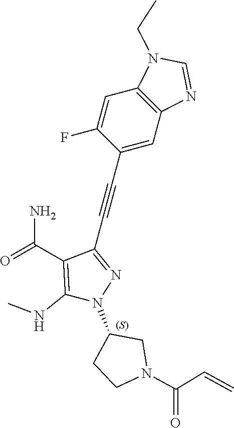 | 3-[2-(1-ethyl-6-fluoro-1,3- benzodiazol-5-yl)ethynyl]-5- (methylamino)-1-[(3S)-1-(prop-2- enoyl)pyrrolidin-3-yl]pyrazole-4- carboxamide |
| 144 | 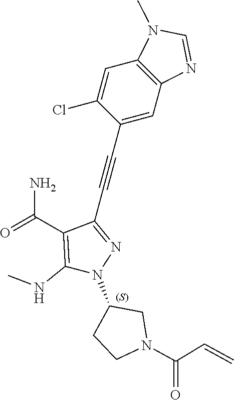 | 3-[2-(6-chloro-1-methyl-1,3- benzodiazol-5-yl)ethynyl]-5- (methylamino)-1-[(3S)-1-(prop-2- enoyl)pyrrolidin-3-yl]pyrazole-4- carboxamide |
| 145 | 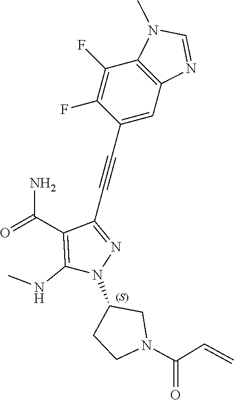 | 3-[2-(6,7-difluoro-1-methyl-1,3- benzodiazol-5-yl)ethynyl]-5- (methylamino)-1-[(3S)-1-(prop-2- enoyl)pyrrolidin-3-yl]pyrazole-4- carboxamide |
| 147 | 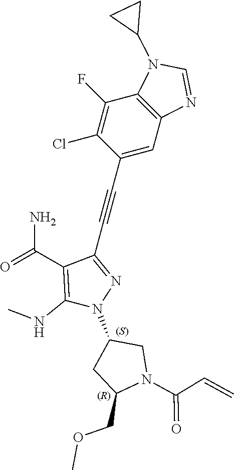 | 3-[2-(6-chloro-1-cyclopropyl-7-fluoro- 1,3-benzodiazol-5-yl)ethynyl]-1- [(3S,5R)-5-(methoxymethyl)-1-(prop- 2-enoyl)pyrrolidin-3-yl]-5- (methylamino)pyrazole-4-carboxamide |
| 148 | 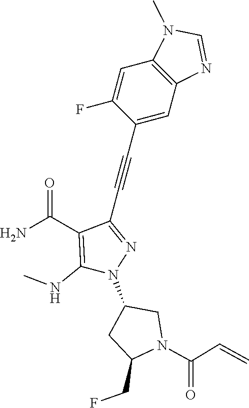 [SMILES: C=CC(=O)N1C[C@@H](n2nc(C#Cc3cc4ncn(C)c4cc3F)c(C(N)=O)c2NC)C[C@@H]1CF] | 3-[2-(6-fluoro-1-methyl-1,3- benzodiazol-5-yl)ethynyl]-1-[(3S,5R)- 5-(fluoromethyl)-1-(prop-2- enoyl)pyrrolidin-3-yl]-5- (methylamino)pyrazole-4-carboxamide |
| 149 | 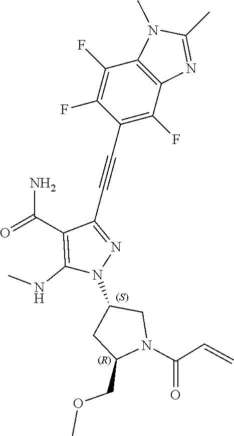 | 1-[(3S,5R)-5-(methoxymethyl)-1- (prop-2-enoyl)pyrrolidin-3-yl]-5- (methylamino)-3-[2-(4,6,7-trifluoro- 1,2-dimethyl-1,3-benzodiazol-5- yl)ethynyl]pyrazole-4-carboxamide |
| 150 | 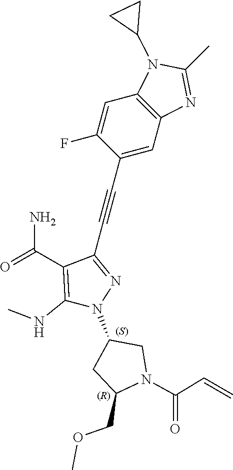 | 3-[2-(1-cyclopropyl-6-fluoro-2- methyl-1,3-benzodiazol-5-yl)ethynyl]- 1-[(3S,5R)-5-(methoxymethyl)-1- (prop-2-enoyl)pyrrolidin-3-yl]-5- (methylamino)pyrazole-4-carboxamide |
| 151 | 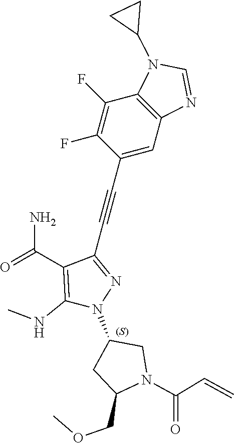 | 3-[2-(1-cyclopropyl-6,7-difluoro-1,3- benzodiazol-5-yl)ethynyl]-1-[(3S,5R)- 5-(methoxymethyl)-1-(prop-2- enoyl)pyrrolidin-3-yl]-5- (methylamino)pyrazole-4-carboxamide |
| 152 | 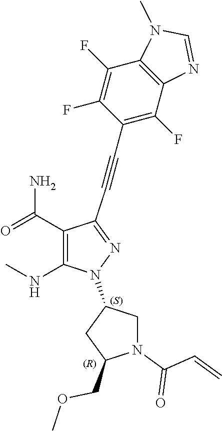 | 1-[(3S,5R)-5-(methoxymethyl)-1- (prop-2-enoyl)pyrrolidin-3-yl]-5- (methylamino)-3-[2-(4,6,7-trifluoro-1- methyl-1,3-benzodiazol-5- yl)ethynyl]pyrazole-4-carboxamide |
| 153 | 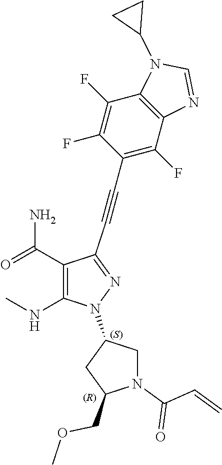 | 3-[2-(1-cyclopropyl-4,6,7-trifluoro- 1,3-benzodiazol-5-yl)ethynyl]-1- [(3S,5R)-5-(methoxymethyl)-1-(prop- 2-enoyl)pyrrolidin-3-yl]-5- (methylamino)pyrazole-4-carboxamide |
| 154 | 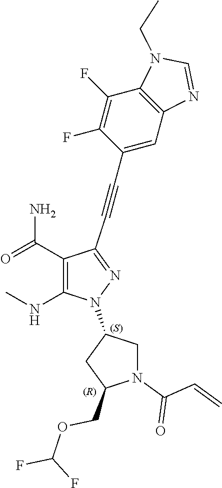 | 1-[(3S,5R)-5- [(difluoromethoxy)methyl]-1-(prop-2- enoyl)pyrrolidin-3-yl]-3-[2-(1-ethyl- 6,7-difluoro-1,3-benzodiazol-5- yl)ethynyl]-5-(methylamino)pyrazole- 4-carboxamide |
| 155 | 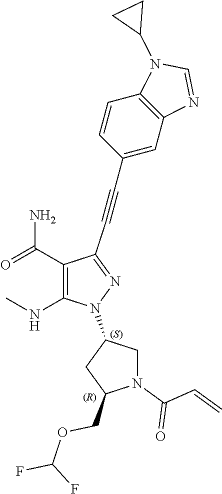 | 3-[2-(1-cyclopropyl-1,3-benzodiazol- 5-yl)ethynyl]-1-[(3S,5R)-5- [(difluoromethoxy)methyl]-1-(prop-2- enoyl)pyrrolidin-3-yl]-5- (methylamino)pyrazole-4-carboxamide |
| 156 | 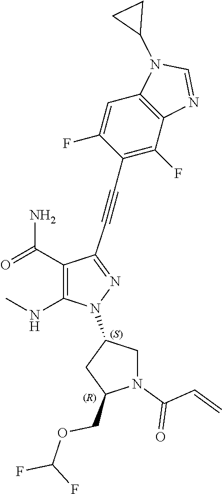 | 1-((3S,5R)-1-acryloyl-5- ((difluoromethoxy)methyl)pyrrolidin- 3-yl)-3-((1-cyclopropyl-4,6-difluoro- 1H-benzo[d]imidazol-5-yl)ethynyl)-5- (methylamino)-1H-pyrazole-4- carboxamide |
| 157 | 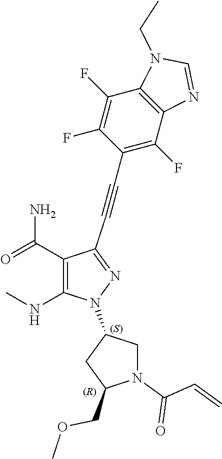 | 3-[2-(1-ethyl-4,6,7-trifluoro-1,3- benzodiazol-5-yl)ethynyl]-1-[(3S,5R)- 5-(methoxymethyl)-1-(prop-2- enoyl)pyrrolidin-3-yl]-5- (methylamino)pyrazole-4-carboxamide |
| 158 | 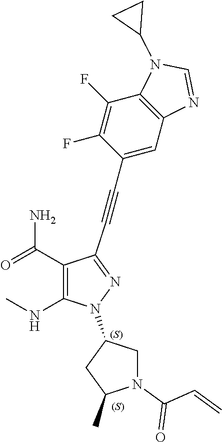 | 3-[2-(1-cyclopropyl-6,7-difluoro-1,3- benzodiazol-5-yl)ethynyl]-1-[(3S,5S)- 5-methyl-1-(prop-2-enoyl)pyrrolidin- 3-yl]-5-(methylamino)pyrazole-4- carboxamide |
| 159 | 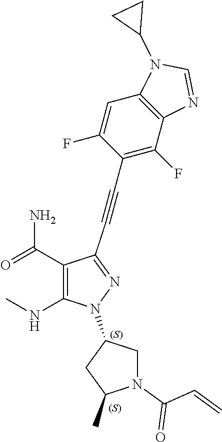 | 3-[2-(1-cyclopropyl-4,6-difluoro-1,3- benzodiazol-5-yl)ethynyl]-1-[(3S,5S)- 5-methyl-1-(prop-2-enoyl)pyrrolidin- 3-yl]-5-(methylamino)pyrazole-4- carboxamide |
| 161 | 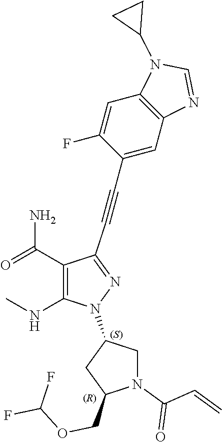 | 1-((3S,5R)-1-acryloyl-5- ((difluoromethoxy)methyl)pyrrolidin- 3-yl)-3-((1-cyclopropyl-6-fluoro-1H- benzo[d]imidazol-5-yl)ethynyl)-5- (methylamino)-1H-pyrazole-4- carboxamide |
| 162 | 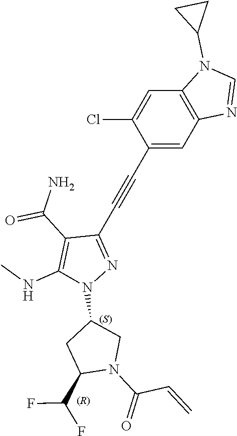 | 3-[2-(6-Chloro-1-cyclopropyl-1,3- benzodiazol-5-yl)ethynyl]-1-[(3S,5R)- 5-(difluoromethyl)-1-(prop-2- enoyl)pyrrolidin-3-yl]-5- (methylamino)pyrazole-4-carboxamide |
| 163 | 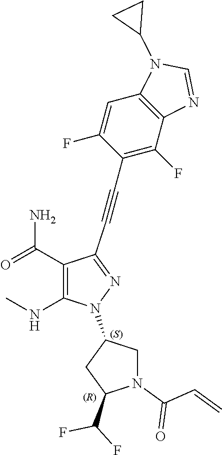 | 3-[2-(1-cyclopropyl-4,6-difluoro-1,3- benzodiazol-5-yl)ethynyl]-1-[(3S,5R)- 5-(difluoromethyl)-1-(prop-2- enoyl)pyrrolidin-3-yl]-5- (methylamino)pyrazole-4-carboxamide |
| 164 | 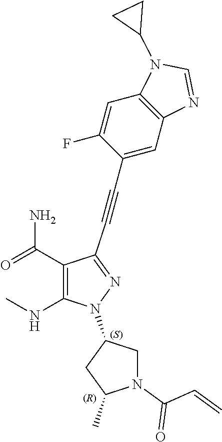 | 1-((3S,5R)-1-acryloyl-5- methylpyrrolidin-3-yl)-3-((1- cyclopropyl-6-fluoro-1H- benzo[d]imidazol-5-yl)ethynyl)-5- (methylamino)-1H-pyrazole-4- carboxamide |
| 165 | 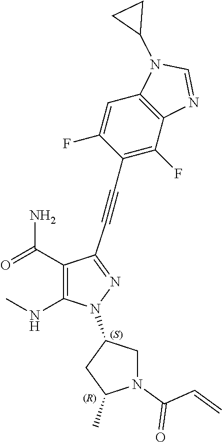 | 3-[2-(1-cyclopropyl-4,6-difluoro-1,3- benzodiazol-5-yl)ethynyl]-1-[(3S,5R)- 5-methyl-1-(prop-2-enoyl)pyrrolidin- 3-yl]-5-(methylamino)pyrazole-4- carboxamide |
| 166 | 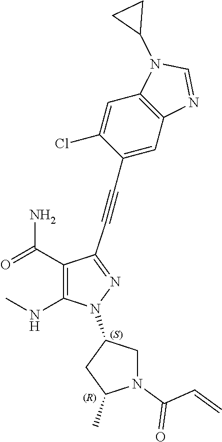 | 1-((3S,5R)-1-acryloyl-5- methylpyrrolidin-3-yl)-3-((6-chloro-1- cyclopropyl-1H-benzo[d]imidazol-5- yl)ethynyl)-5-(methylamino)-1H- pyrazole-4-carboxamide |
| 167 | 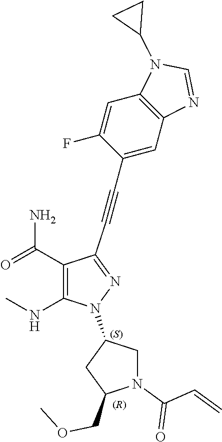 | 5-amino-3-[2-(1-cyclopropyl-6-fluoro- 1,3-benzodiazol-5-yl)ethynyl]-1- [(3S,5R)-5-(methoxymethyl)-1-(prop- 2-enoyl)pyrrolidin-3-yl]pyrazole-4- carboxamide |
| 168 | 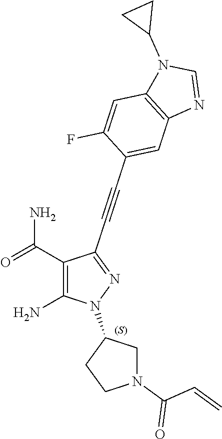 | 5-amino-3-[2-(1-cyclopropyl-6-fluoro- 1,3-benzodiazol-5-yl)ethynyl]-1-[(3S)- 1-(prop-2-enoyl)pyrrolidin-3- yl]pyrazole-4-carboxamide |
| 169 | 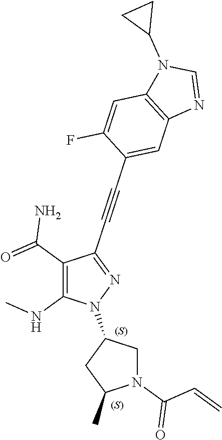 | 1-((3S,5S)-1-acryloyl-5- methylpyrrolidin-3-yl)-3-((1- cyclopropyl-6-fluoro-1H- benzo[d]imidazol-5-yl)ethynyl)-5- (methylamino)-1H-pyrazole-4- carboxamide |
| 170 | 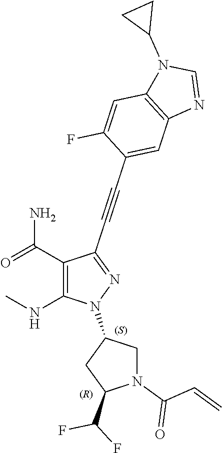 | 3-[2-(1-cyclopropyl-6-fluoro-1,3- benzodiazol-5-yl)ethynyl]-1-[(3S,5R)- 5-(difluoromethyl)-1-(prop-2- enoyl)pyrrolidin-3-yl]-5- (methylamino)pyrazole-4-carboxamide |
| 171 | 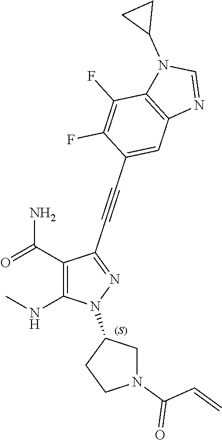 | 3-[2-(1-cyclopropyl-6,7-difluoro-1,3- benzodiazol-5-yl)ethynyl]-5- (methylamino)-1-[(3S)-1-(prop-2- enoyl)pyrrolidin-3-yl]pyrazole-4- carboxamide |
| 172 | 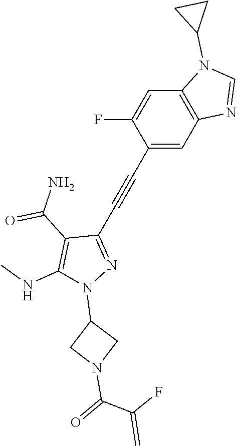 [SMILES: C=C(F)C(=O)N1CC(n2nc(C#Cc3cc4ncn(C5CC5)c4cc3F)c(C(N)=O)c2NC)C1] | 3-((1-cyclopropyl-6-fluoro-1H- benzo[d]imidazol-5-yl)ethynyl)-1-(1- (2-fluoroacryloyl)azetidin-3-yl)-5- (methylamino)-1H-pyrazole-4- carboxamide |
| 173 | 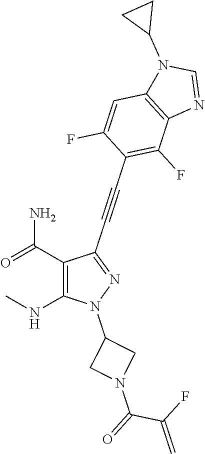 [SMILES: C=C(F)C(=O)N1CC(n2nc(C#Cc3c(F)cc4c(ncn4C4CC4)c3F)c(C(N)=O)c2NC)C1] | 3-((1-cyclopropyl-4,6-difluoro-1H- benzo[d]imidazol-5-yl)ethynyl)-1-(1- (2-fluoroacryloyl)azetidin-3-yl)-5- (methylamino)-1H-pyrazole-4- carboxamide |
| 174 | 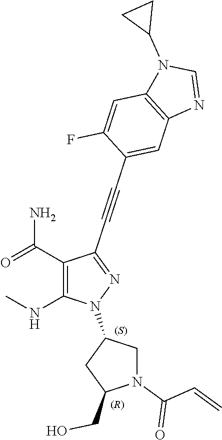 | 1-((3S,5R)-1-acryloyl-5- (hydroxymethyl)pyrrolidin-3-yl)-3-((1- cyclopropyl-6-fluoro-1H- benzo[d]imidazol-5-yl)ethynyl)-5- (methylamino)-1H-pyrazole-4- carboxamide |
| 175 | 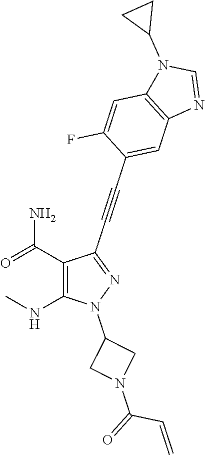 [SMILES: C=CC(=O)N1CC(n2nc(C#Cc3cc4ncn(C5CC5)c4cc3F)c(C(N)=O)c2NC)C1] | tert-butyl 3-[2-(1-cyclopropyl-6- fluoro-1,3-benzodiazol-5-yl)ethynyl]- 5-(methylamino)-1-[1-(prop-2- enoyl)azetidin-3-yl]pyrazole-4- carboxamide |
| 176 | 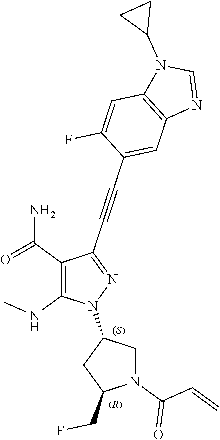 | 1-((3S,5R)-1-acryloyl-5- (fluoromethyl)pyrrolidin-3-yl)-3-((1- cyclopropyl-6-fluoro-1H- benzo[d]imidazol-5-yl)ethynyl)-5- (methylamino)-1H-pyrazole-4- carboxamide |
| 177 | 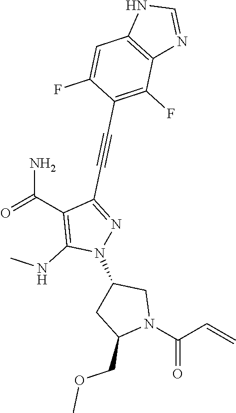 [SMILES: C=CC(=O)N1C[C@@H](n2nc(C#Cc3c(F)cc4[nH]cnc4c3F)c(C(N)=O)c2NC)C[C@@H]1COC] | 1-((3S,5R)-1-acryloyl-5- (methoxymethyl)pyrrolidin-3-yl)-3- ((4,6-difluoro-1H-benzo[d]imidazol-5- yl)ethynyl)-5-(methylamino)-1H- pyrazole-4-carboxamide |
| 178 | 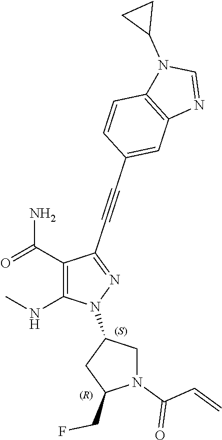 | 1-((3S,5R)-1-acryloyl-5- (fluoromethyl)pyrrolidin-3-yl)-3-((1- cyclopropyl-1H-benzo[d]imidazol-5- yl)ethynyl)-5-(methylamino)-1H- pyrazole-4-carboxamide |
| 179 | 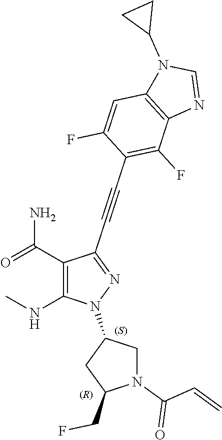 | 1-((3S,5R)-1-acryloyl-5- (fluoromethyl)pyrrolidin-3-yl)-3-((1- cyclopropyl-4,6-difluoro-1H- benzo[d]imidazol-5-yl)ethynyl)-5- (methylamino)-1H-pyrazole-4- carboxamide |
| 180 | 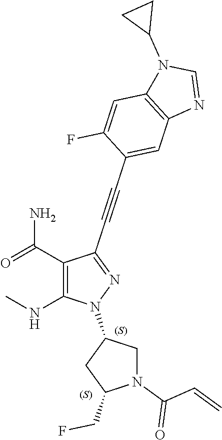 | 3-[2-(1-cyclopropyl-6-fluoro-1,3- benzodiazol-5-yl)ethynyl]-1-[(3S,5S)- 5-(fluoromethyl)-1-(prop-2- enoyl)pyrrolidin-3-yl]-5- (methylamino)pyrazole-4-carboxamide |
| 181 | 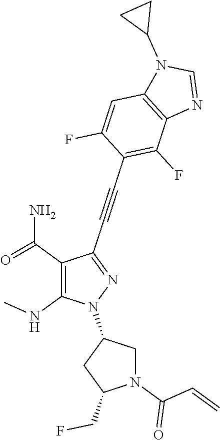 [SMILES: C=CC(=O)N1C[C@@H](n2nc(C#Cc3c(F)cc4c(ncn4C4CC4)c3F)c(C(N)=O)c2NC)C[C@H]1CF] | 3-[2-(1-cyclopropyl-4,6-difluoro-1,3- benzodiazol-5-yl)ethynyl]-1-[(3S,5S)- 5-(fluoromethyl)-1-(prop-2- enoyl)pyrrolidin-3-yl]-5- (methylamino)pyrazole-4-carboxamide |
| 182 | 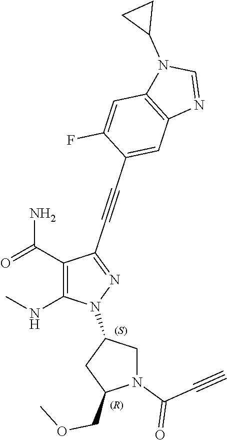 | 3-((1-cyclopropyl-6-fluoro-1H- benzo[d]imidazol-5-yl)ethynyl)-1- ((3S,5R)-5-(methoxymethyl)-1- propioloylpyrrolidin-3-yl)-5- (methylamino)-1H-pyrazole-4- carboxamide |
| 183 | 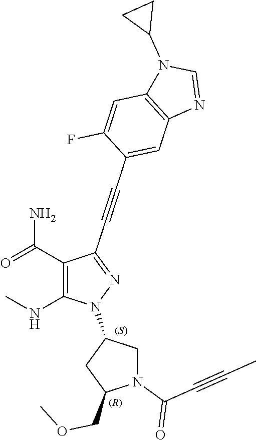 | 1-((3S,5R)-1-(but-2-ynoyl)-5- (methoxymethyl)pyrrolidin-3-yl)-3- ((1-cyclopropyl-6-fluoro-1H- benzo[d]imidazol-5-yl)ethynyl)-5- (methylamino)-1H-pyrazole-4- carboxamide |
| 184 | 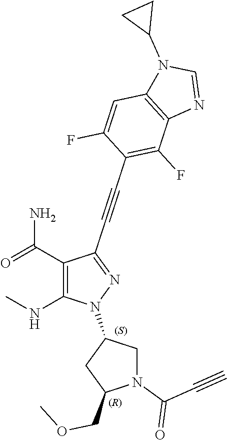 | 3-[2-(1-cyclopropyl-4,6-difluoro-1,3- benzodiazol-5-yl)ethynyl]-1-[(3S,5R)- 5-(methoxymethyl)-1-(prop-2- ynoyl)pyrrolidin-3-yl]-5- (methylamino)pyrazole-4-carboxamide |
| 185 | 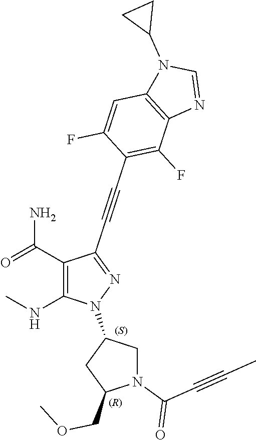 | 1-[(3S,5R)-1-(but-2-ynoyl)-5- (methoxymethyl)pyrrolidin-3-yl]-3-[2- (1-cyclopropyl-4,6-difluoro-1,3- benzodiazol-5-yl)ethynyl]-5- (methylamino)pyrazole-4-carboxamide |
| 186 | 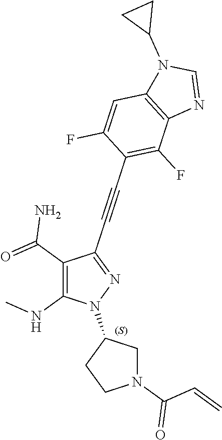 | 3-[2-(1-cyclopropyl-4,6-difluoro-1,3- benzodiazol-5-yl)ethynyl]-5- (methylamino)-1-[(3S)-1-(prop-2- enoyl)pyrrolidin-3-yl]pyrazole-4- carboxamide |
| 187 | 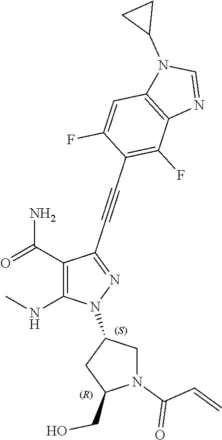 | 3-[2-(1-cyclopropyl-4,6-difluoro-1,3- benzodiazol-5-yl)ethynyl]-1-[(3S,5R)- 5-(hydroxymethyl)-1-(prop-2- enoyl)pyrrolidin-3-yl]-5- (methylamino)pyrazole-4-carboxamide |
| 188 | 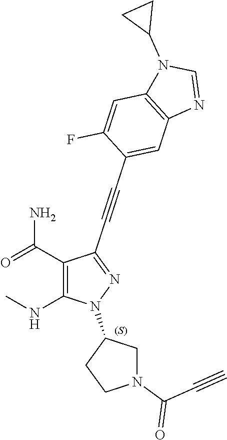 | 3-[2-(1-cyclopropyl-6-fluoro-1,3- benzodiazol-5-yl)ethynyl]-5- (methylamino)-1-[1-(prop-2- ynoyl)pyrrolidin-3-yl]pyrazole-4- carboxamide |
| 189 | 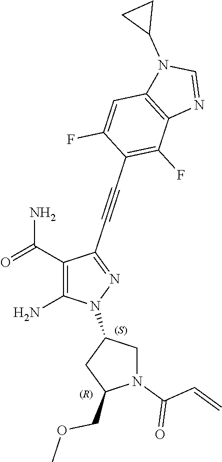 | 5-amino-3-[2-(1-cyclopropyl-4,6- difluoro-1,3-benzodiazol-5- yl)ethynyl]-1-[(3S,5R)-5- (methoxymethyl)-1-(prop-2- enoyl)pyrrolidin-3-yl]pyrazole-4- carboxamide |
| 190 | 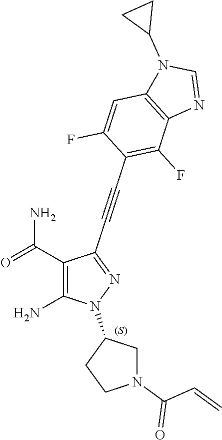 | 5-amino-3-[2-(1-cyclopropyl-4,6- difluoro-1,3-benzodiazol-5- yl)ethynyl]-1-[(3S)-1-(prop-2- enoyl)pyrrolidin-3-yl]pyrazole-4- carboxamide |
| 191 | 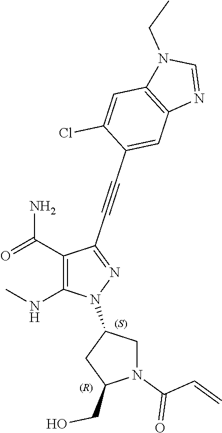 | 3-[2-(6-chloro-1-ethyl-1,3- benzodiazol-5-yl)ethynyl]-1-[(3S,5R)- 5-(hydroxymethyl)-1-(prop-2- enoyl)pyrrolidin-3-yl]-5- (methylamino)pyrazole-4-carboxamide |
| 192 | 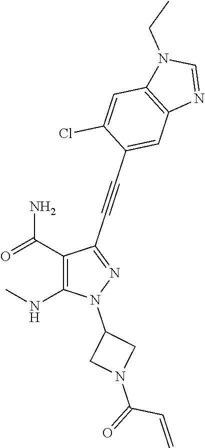 [SMILES: C=CC(=O)N1CC(n2nc(C#Cc3cc4ncn(CC)c4cc3Cl)c(C(N)=O)c2NC)C1] | 3-[2-(6-chloro-1-ethyl-1,3- benzodiazol-5-yl)ethynyl]-5- (methylamino)-1-[1-(prop-2- enoyl)azetidin-3-yl]pyrazole-4- carboxamide |
| 193 | 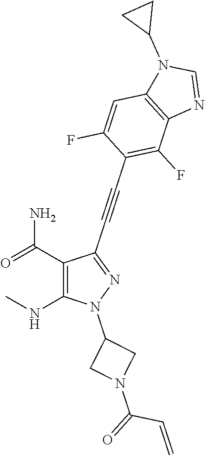 [SMILES: C=CC(=O)N1CC(n2nc(C#Cc3c(F)cc4c(ncn4C4CC4)c3F)c(C(N)=O)c2NC)C1] | 3-[2-(1-cyclopropyl-4,6-difluoro-1,3- benzodiazol-5-yl)ethynyl]-5- (methylamino)-1-[1-(prop-2- enoyl)azetidin-3-yl]pyrazole-4- carboxamide |
| 194 | 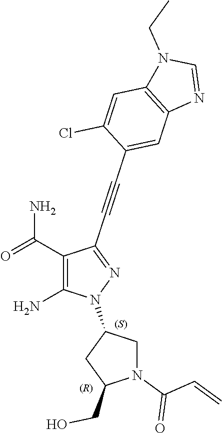 | 5-amino-3-[2-(6-chloro-1-ethyl-1,3- benzodiazol-5-yl)ethynyl]-1-[(3S,5R)- 5-(hydroxymethyl)-1-(prop-2- enoyl)pyrrolidin-3-yl]pyrazole-4- carboxamide |
| 195 | 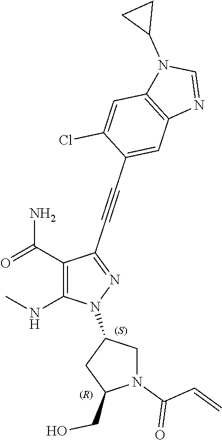 | 3-[2-(6-chloro-1-cyclopropyl-1,3- benzodiazol-5-yl)ethynyl]-1-[(3S,5R)- 5-(hydroxymethyl)-1-(prop-2- enoyl)pyrrolidin-3-yl]-5- (methylamino)pyrazole-4-carboxamide |
| 196 | 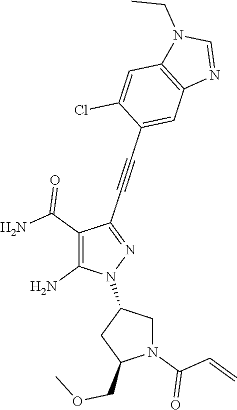 [SMILES: C=CC(=O)N1C[C@@H](n2nc(C#Cc3cc4ncn(CC)c4cc3Cl)c(C(N)=O)c2N)C[C@@H]1COC] | 5-amino-3-[2-(6-chloro-1-ethyl-1,3- benzodiazol-5-yl)ethynyl]-1-[(3S,5R)- 5-(methoxymethyl)-1-(prop-2- enoyl)pyrrolidin-3-yl]pyrazole-4- carboxamide |
| 197 | 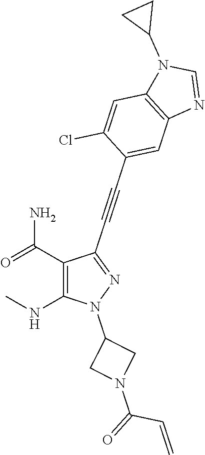 [SMILES: C=CC(=O)N1CC(n2nc(C#Cc3cc4ncn(C5CC5)c4cc3Cl)c(C(N)=O)c2NC)C1] | 3-[2-(6-chloro-1-cyclopropyl-1,3- benzodiazol-5-yl)ethynyl]-5- (methylamino)-1-[1-(prop-2- enoyl)azetidin-3-yl]pyrazole-4- carboxamide |
| 198 | 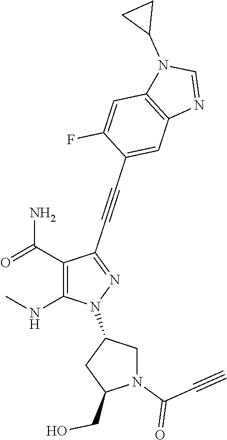 [SMILES: C#CC(=O)N1C[C@@H](n2nc(C#Cc3cc4ncn(C5CC5)c4cc3F)c(C(N)=O)c2NC)C[C@@H]1CO] | 3-[2-(1-cyclopropyl-6-fluoro-1,3- benzodiazol-5-yl)ethynyl]-1-[(3S,5R)- 5-(hydroxymethyl)-1-(prop-2- ynoyl)pyrrolidin-3-yl]-5- (methylamino)pyrazole-4-carboxamide |
| 199 | 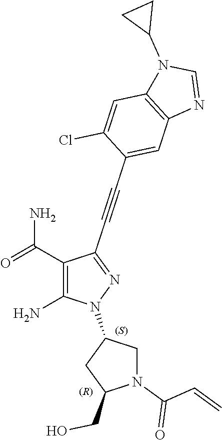 | 5-amino-3-[2-(6-chloro-1-cyclopropyl- 1,3-benzodiazol-5-yl)ethynyl]-1- [(3S,5R)-5-(hydroxymethyl)-1-(prop-2- enoyl)pyrrolidin-3-yl]pyrazole-4- carboxamide |
| 200 | 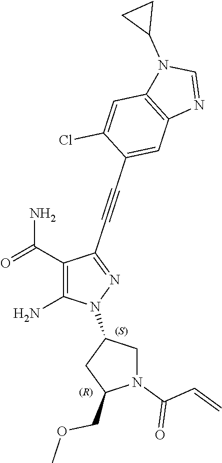 | 5-amino-3-[2-(6-chloro-1-cyclopropyl- 1,3-benzodiazol-5-yl)ethynyl]-1- [(3S,5R)-5-(methoxymethyl)-1-(prop- 2-enoyl)pyrrolidin-3-yl]pyrazole-4- carboxamide |
| 201 | 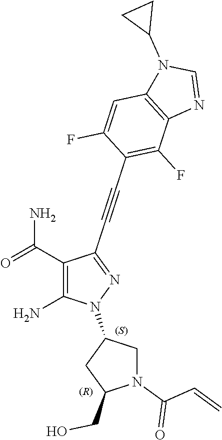 | 5-amino-3-[2-(1-cyclopropyl-4,6- difluoro-1,3-benzodiazol-5- yl)ethynyl]-1-[(3S,5R)-5- (hydroxymethyl)-1-(prop-2- enoyl)pyrrolidin-3-yl]pyrazole-4- carboxamide |
| 202 | 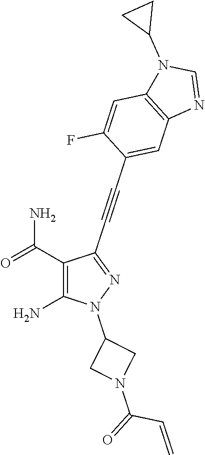 [SMILES: C=CC(=O)N1CC(n2nc(C#Cc3cc4ncn(C5CC5)c4cc3F)c(C(N)=O)c2N)C1] | 5-amino-3-[2-(1-cyclopropyl-6-fluoro- 1,3-benzodiazol-5-yl)ethynyl]-1-[1- (prop-2-enoyl)azetidin-3-yl]pyrazole- 4-carboxamide |
| 203 | 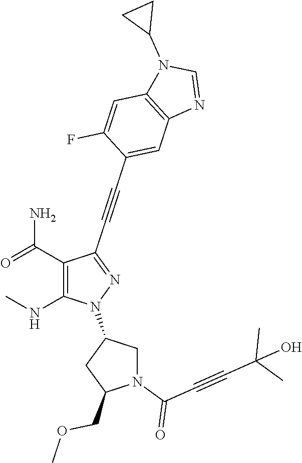 [SMILES: CNc1c(C(N)=O)c(C#Cc2cc3ncn(C4CC4)c3cc2F)nn1[C@H]1C[C@H](COC)N(C(=O)C#CC(C)(C)O)C1] | 3-((1-cyclopropyl-6-fluoro-1H- benzo[d]imidazol-5-yl)ethynyl)-1- ((3S,5R)-1-(4-hydroxy-4-methylpent- 2-ynoyl)-5- (methoxymethyl)pyrrolidin-3-yl)-5- (methylamino)-1H-pyrazole-4- carboxamide |
| 204 | 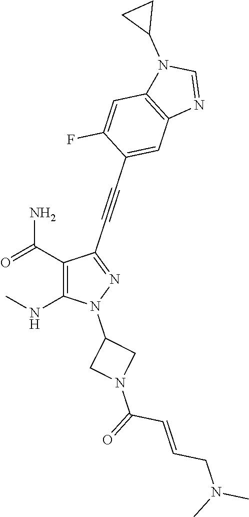 [SMILES: CNc1c(C(N)=O)c(C#Cc2cc3ncn(C4CC4)c3cc2F)nn1C1CN(C(=O)/C=C/CN(C)C)C1] | 3-[2-(1-cyclopropyl-6-fluoro-1,3- benzodiazol-5-yl)ethynyl]-1-{1-[(2E)- 4-dimethylamino)but-2- enoyl]azetidin-3-yl}-5- (methylamino)pyrazole-4-carboxamide |
| 205 | 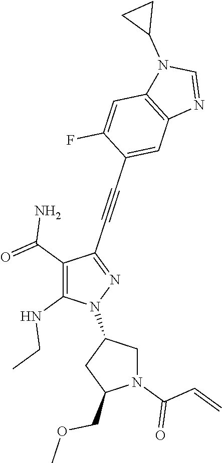 [SMILES: C=CC(=O)N1C[C@@H](n2nc(C#Cc3cc4ncn(C5CC5)c4cc3F)c(C(N)=O)c2NCC)C[C@@H]1COC] | 1-((3S,5R)-1-acryloyl-5- (methoxymethyl)pyrrolidin-3-yl)-3- ((1-cyclopropyl-6-fluoro-1H- benzo[d]imidazol-5-yl)ethynyl)-5- (ethylamino)-1H-pyrazole-4- carboxamide |
| 206 | 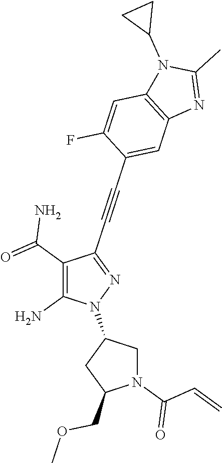 [SMILES: C=CC(=O)N1C[C@@H](n2nc(C#Cc3cc4nc(C)n(C5CC5)c4cc3F)c(C(N)=O)c2N)C[C@@H]1COC] | 5-amino-3-[2-(1-cyclopropyl-6-fluoro- 2-methyl-1,3-benzodiazol-5- yl)ethynyl]-1-[(3S,5R)-5- (methoxymethyl)-1-(prop-2- enoyl)pyrrolidin-3-yl]pyrazole-4- carboxamide |
| 207 | 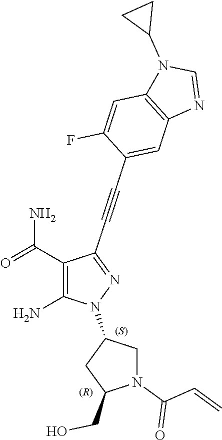 | 5-amino-3-[2-(1-cyclopropyl-6-fluoro- 1,3-benzodiazol-5-yl)ethynyl]-1- [(3S,5R)-5-(hydroxymethyl)-1-(prop-2- enoyl)pyrrolidin-3-yl]pyrazole-4- carboxamide |
| 208 | 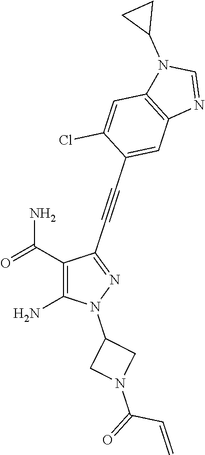 [SMILES: C=CC(=O)N1CC(n2nc(C#Cc3cc4ncn(C5CC5)c4cc3Cl)c(C(N)=O)c2N)C1] | 5-amino-3-[2-(6-chloro-1-cyclopropyl- 1,3-benzodiazol-5-yl)ethynyl]-1-[1- (prop-2-enoyl)azetidin-3-yl]pyrazole- 4-carboxamide |
| 209 | 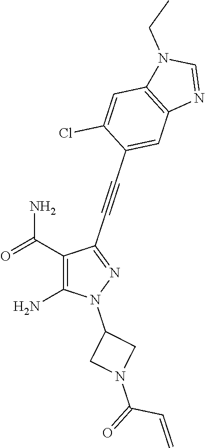 [SMILES: C=CC(=O)N1CC(n2nc(C#Cc3cc4ncn(CC)c4cc3Cl)c(C(N)=O)c2N)C1] | 5-amino-3-[2-(6-chloro-1-ethyl-1,3- benzodiazol-5-yl)ethynyl]-1-[1-(prop- 2-enoyl)azetidin-3-yl]pyrazole-4- carboxamide |
| 210 | 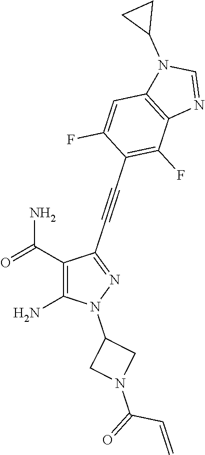 [SMILES: C=CC(=O)N1CC(n2nc(C#Cc3c(F)cc4c(ncn4C4CC4)c3F)c(C(N)=O)c2N)C1] | 5-amino-3-[2-(1-cyclopropyl-4,6- difluoro-1,3-benzodiazol-5- yl)ethynyl]-1-[1-(prop-2- enoyl)azetidin-3-yl]pyrazole-4- carboxamide |
| 211 | 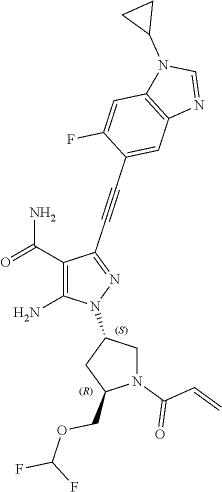 | 5-amino-3-[2-(1-cyclopropyl-6-fluoro- 1,3-benzodiazol-5-yl)ethynyl]-1- [(3S,5R)-5-[(difluoromethoxy)methyl]- 1-(prop-2-enoyl)pyrrolidin-3- yl]pyrazole-4-carboxamide |
| 212 | 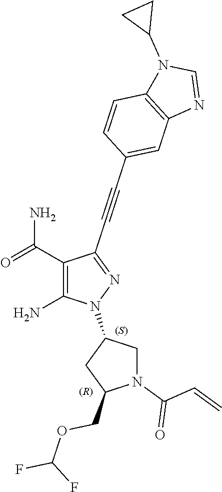 | 5-amino-3-[2-(1-cyclopropyl-1,3- benzodiazol-5-yl)ethynyl]-1-[(3S,5R)- 5-[(difluoromethoxy)methyl]-1-(prop- 2-enoyl)pyrrolidin-3-yl]pyrazole-4- carboxamide |
| 213 | 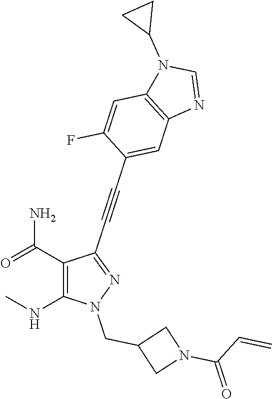 [SMILES: C=CC(=O)N1CC(Cn2nc(C#Cc3cc4ncn(C5CC5)c4cc3F)c(C(N)=O)c2NC)C1] | 3-[2-(1-cyclopropyl-6-fluoro-1,3- benzodiazol-5-yl)ethynyl]-5- (methylamino)-1-{[1-(prop-2- enoyl)azetidin-3-yl]methyl}pyrazole- 4-carboxamide |
| 214 | 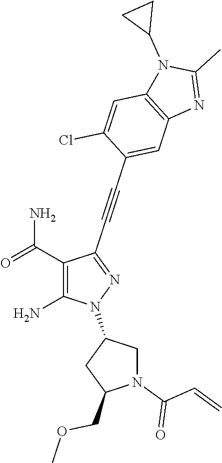 [SMILES: C=CC(=O)N1C[C@@H](n2nc(C#Cc3cc4nc(C)n(C5CC5)c4cc3Cl)c(C(N)=O)c2N)C[C@@H]1COC] | 5-amino-3-[2-(6-chloro-1-cyclopropyl- 2-methyl-1,3-benzodiazol-5- yl)ethynyl]-1-[(3S,5R)-5- (methoxymethyl)-1-(prop-2- enoyl)pyrrolidin-3-yl]pyrazole-4- carboxamide |
| 215 | 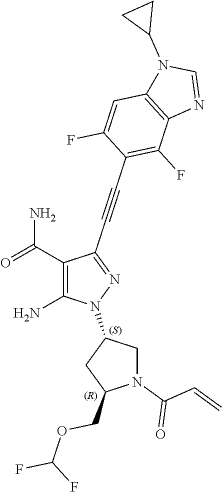 | 5-amino-3-[2-(1-cyclopropyl-4,6- difluoro-1,3-benzodiazol-5- yl)ethynyl]-1-[(3S,5R)-5- [(difluoromethoxy)methyl]-1-(prop-2- enoyl)pyrrolidin-3-yl]pyrazole-4- carboxamide |
| 216 | 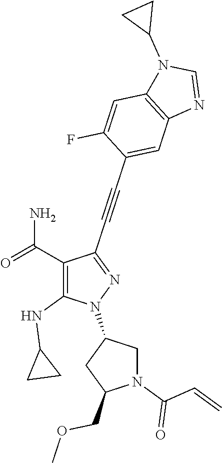 [SMILES: C=CC(=O)N1C[C@@H](n2nc(C#Cc3cc4ncn(C5CC5)c4cc3F)c(C(N)=O)c2NC2CC2)C[C@@H]1COC] | 3-[2-(1-cyclopropyl-6-fluoro-1,3- benzodiazol-5-yl)ethynyl]-5- (cyclopropylamino)-1-[(3S,5R)-5- (methoxymethyl)-1-(prop-2- enoyl)pyrrolidin-3-yl]pyrazole-4- carboxamide |
| 217 | 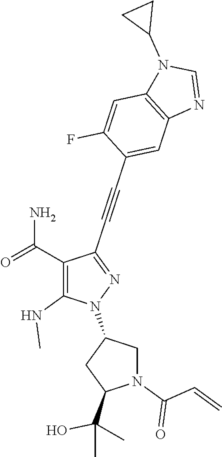 [SMILES: C=CC(=O)N1C[C@@H](n2nc(C#Cc3cc4ncn(C5CC5)c4cc3F)c(C(N)=O)c2NC)C[C@@H]1C(C)(C)O] | 3-[2-(1-cyclopropyl-6-fluoro-1,3- benzodiazol-5-yl)ethynyl]-1-[(3S,5R)- 5-(2-hydroxypropan-2-yl)-1-(prop-2- enoyl)pyrrolidin-3-yl]-5- (methylamino)pyrazole-4-carboxamide |
| 218 | 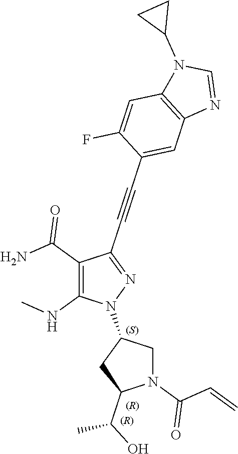 | 3-[2-(1-cyclopropyl-6-fluoro-1,3- benzodiazol-5-yl)ethynyl]-1-[(3S,5R)- 5-[(1R)-1-hydroxyethyl]-1-(prop-2- enoyl)pyrrolidin-3-yl]-5- (methylamino)pyrazole-4-carboxamide |
| 219 | 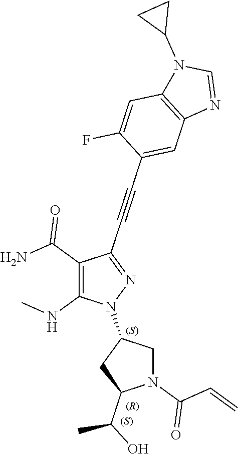 | 3-[2-(1-cyclopropyl-6-fluoro-1,3- benzodiazol-5-yl)ethynyl]-1-[(3S,5R)- 5-[(1S)-1-hydroxyethyl]-1-(prop-2- enoyl)pyrrolidin-3-yl]-5- (methylamino)pyrazole-4-carboxamide |
| 220 | 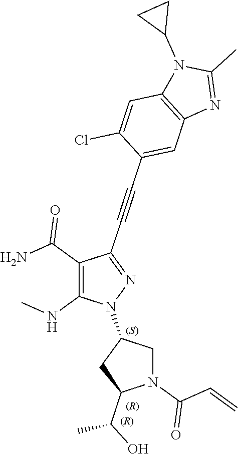 | 1-((3S,5R)-1-acryloyl-5-((R)-1- hydroxyethyl)pyrrolidin-3-yl)-3-((6- chloro-1-cyclopropyl-2-methyl-1H- benzo[d]imidazol-5-yl)ethynyl)-5- (methylamino)-1H-pyrazole-4- carboxamide |
| 221 | 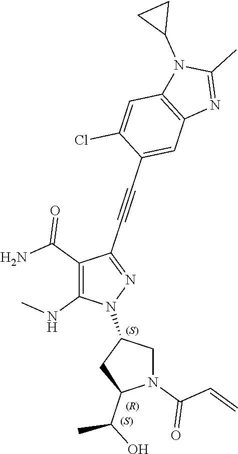 | 1-((3S,5R)-1-acryloyl-5-((S)-1- hydroxyethyl)pyrrolidin-3-yl)-3-((6- chloro-1-cyclopropyl-2-methyl-1H- benzo[d]imidazol-5-yl)ethynyl)-5- (methylamino)-1H-pyrazole-4- carboxamide |
| 222 | 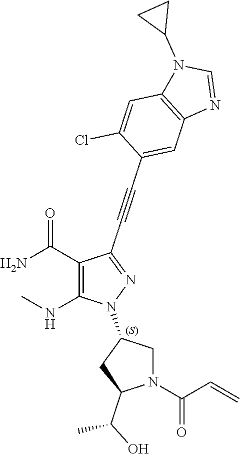 | 3-[2-(6-chloro-1-cyclopropyl-1,3- benzodiazol-5-yl)ethynyl]-1-[(3S,5R)- 5-[(1R)-1-hydroxyethyl]-1-(prop-2- enoyl)pyrrolidin-3-yl]-5- (methylamino)pyrazole-4-carboxamide |
| 223 | 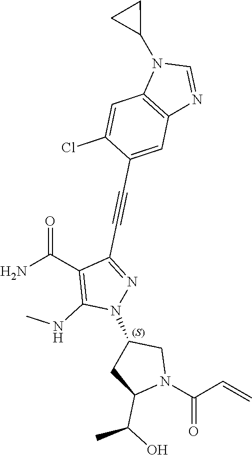 | 3-[2-(6-Chloro-1-cyclopropyl-1,3- benzodiazol-5-yl)ethynyl]-1-[(3S,5R)- 5-[(1S)-1-hydroxyethyl]-1-(prop-2- enoyl)pyrrolidin-3-yl]-5- (methylamino)pyrazole-4-carboxamide |
| 224 | 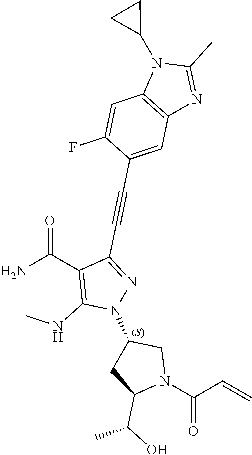 | 3-[2-(1-cyclopropyl-6-fluoro-2- methyl-1,3-benzodiazol-5-yl)ethynyl]- 1-[(3S,5R)-5-[(1R)-1-hydroxyethyl]-1- (prop-2-enoyl)pyrrolidin-3-yl]-5- (methylamino)pyrazole-4-carboxamide |
| 225 | 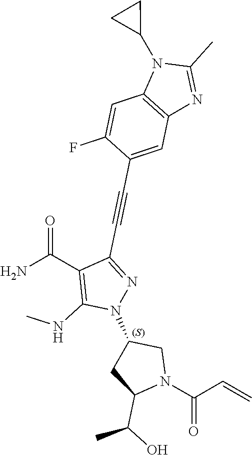 | 3-[2-(1-cyclopropyl-6-fluoro-2- methyl-1,3-benzodiazol-5-yl)ethynyl]- 1-[(3S,5R)-5-[(1S)-1-hydroxyethyl]-1- (prop-2-enoyl)pyrrolidin-3-yl]-5- (methylamino)pyrazole-4-carboxamide |
| 226 | 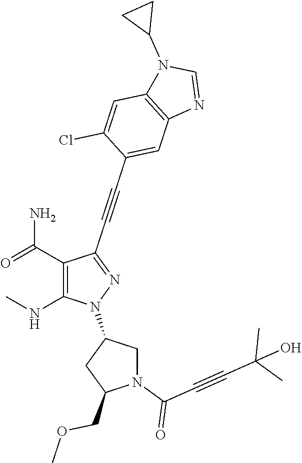 [SMILES: CNc1c(C(N)=O)c(C#Cc2cc3ncn(C4CC4)c3cc2Cl)nn1[C@H]1C[C@H](COC)N(C(=O)C#CC(C)(C)O)C1] | 3-[2-(6-chloro-1-cyclopropyl-1,3- benzodiazol-5-yl)ethynyl]-1-[(3S,5R)- 1-(4-hydroxy-4-methylpent-2-ynoyl)- 5-(methoxymethyl)pyrrolidin-3-yl]-5- (methylamino)pyrazole-4-carboxamide |
| 227 | 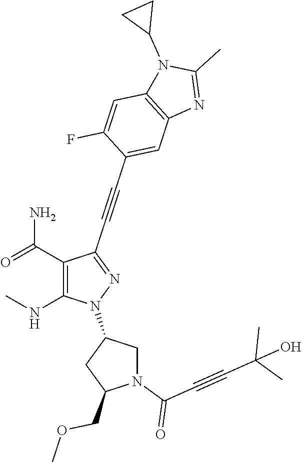 [SMILES: CNc1c(C(N)=O)c(C#Cc2cc3nc(C)n(C4CC4)c3cc2F)nn1[C@H]1C[C@H](COC)N(C(=O)C#CC(C)(C)O)C1] | 3-[2-(1-cyclopropyl-6-fluoro-2- methyl-1,3-benzodiazol-5-yl)ethynyl]- 1-[(3S,5R)-1-(4-hydroxy-4- methylpent-2-ynoyl)-5- (methoxymethyl)pyrrolidin-3-yl]-5- (methylamino)pyrazole-4-carboxamide |
| 228 | 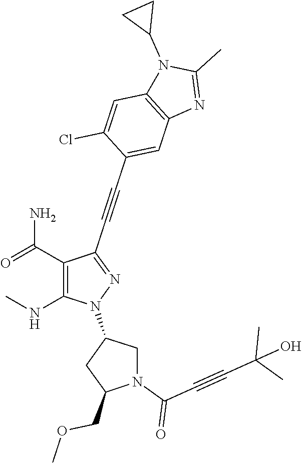 [SMILES: CNc1c(C(N)=O)c(C#Cc2cc3nc(C)n(C4CC4)c3cc2Cl)nn1[C@H]1C[C@H](COC)N(C(=O)C#CC(C)(C)O)C1] | 3-[2-(6-chloro-1-cyclopropyl-2- methyl-1,3-benzodiazol-5-yl)ethynyl]- 1-[(3S,5R)-1-(4-hydroxy-4- methylpent-2-ynoyl)-5- (methoxymethyl)pyrrolidin-3-yl]-5- (methylamino)pyrazole-4-carboxamide |
| 230 | 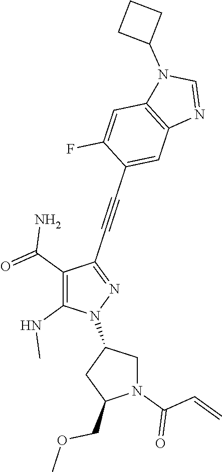 [SMILES: C=CC(=O)N1C[C@@H](n2nc(C#Cc3cc4ncn(C5CCC5)c4cc3F)c(C(N)=O)c2NC)C[C@@H]1COC] | 1-((3S,5R)-1-acryloyl-5- (methoxymethyl)pyrrolidin-3-yl)-3- ((1-cyclobutyl-6-fluoro-1H- benzo[d]imidazol-5-yl)ethynyl)-5- (methylamino)-1H-pyrazole-4- carboxamide |
| 231 | 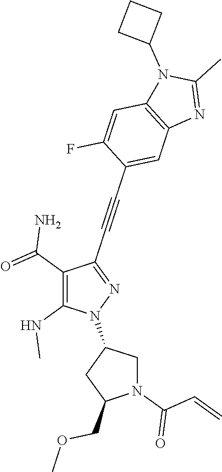 [SMILES: C=CC(=O)N1C[C@@H](n2nc(C#Cc3cc4nc(C)n(C5CCC5)c4cc3F)c(C(N)=O)c2NC)C[C@@H]1COC] | 1-((3S,5R)-1-acryloyl-5- (methoxymethyl)pyrrolidin-3-yl)-3- ((1-cyclobutyl-6-fluoro-2-methyl-1H- benzo[d]imidazol-5-yl)ethynyl)-5- (methylamino)-1H-pyrazole-4- carboxamide |
| 232 | 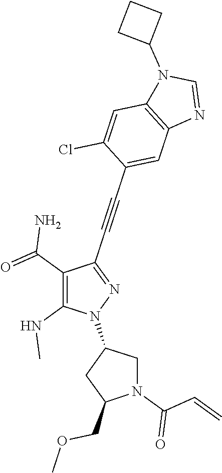 [SMILES: C=CC(=O)N1C[C@@H](n2nc(C#Cc3cc4ncn(C5CCC5)c4cc3Cl)c(C(N)=O)c2NC)C[C@@H]1COC] | 3-[2-(6-chloro-1-cyclobutyl-1,3- benzodiazol-5-yl)ethynyl]-1-[(3S,5R)- 5-(methoxymethyl)-1-(prop-2- enoyl)pyrrolidin-3-yl]-5- (methylamino)pyrazole-4-carboxamide |
| 233 | 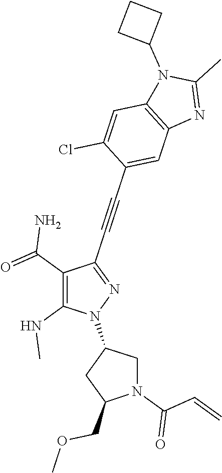 [SMILES: C=CC(=O)N1C[C@@H](n2nc(C#Cc3cc4nc(C)n(C5CCC5)c4cc3Cl)c(C(N)=O)c2NC)C[C@@H]1COC] | 1-((3S,5R)-1-acryloyl-5- (methoxymethyl)pyrrolidin-3-yl)-3- ((6-chloro-1-cyclobutyl-2-methyl-1H- benzo[d]imidazol-5-yl)ethynyl)-5- (methylamino)-1H-pyrazole-4- carboxamide |
| 234 | 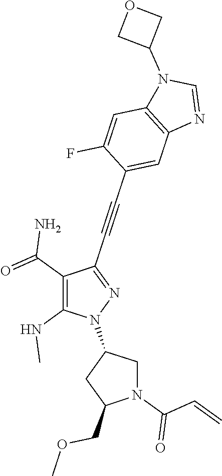 [SMILES: C=CC(=O)N1C[C@@H](n2nc(C#Cc3cc4ncn(C5COC5)c4cc3F)c(C(N)=O)c2NC)C[C@@H]1COC] | 3-{2-[6-fluoro-1-(oxetan-3-yl)-1,3- benzodiazol-5-yl]ethynyl}-1-[(3S,5R)- 5-(methoxymethyl)-1-(prop-2- enoyl)pyrrolidin-3-yl]-5- (methylamino)pyrazole-4-carboxamide |
| 235 | 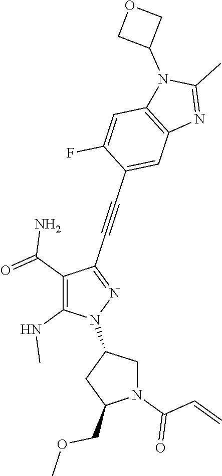 [SMILES: C=CC(=O)N1C[C@@H](n2nc(C#Cc3cc4nc(C)n(C5COC5)c4cc3F)c(C(N)=O)c2NC)C[C@@H]1COC] | 3-{2-[6-fluoro-2-methyl-1-(oxetan-3- yl)-1,3-benzodiazol-5-yl]ethynyl}-1- [(3S,5R)-5-(methoxymethyl)-1-(prop- 2-enoyl)pyrrolidin-3-yl]-5- (methylamino)pyrazole-4-carboxamide |
| 236 | 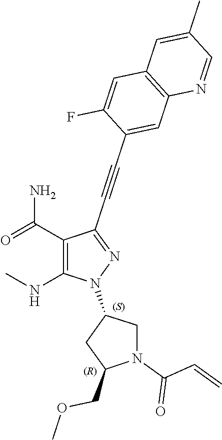 | 3-[2-(6-fluoro-3-methylquinolin-7- yl)ethynyl]-1-[(3S,5R)-5- (methoxymethyl)-1-(prop-2- enoyl)pyrrolidin-3-yl]-5- (methylamino)pyrazole-4-carboxamide |
| 237 | 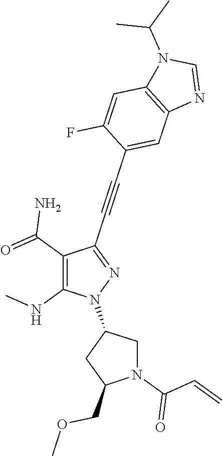 [SMILES: C=CC(=O)N1C[C@@H](n2nc(C#Cc3cc4ncn(C(C)C)c4cc3F)c(C(N)=O)c2NC)C[C@@H]1COC] | 1-((3S,5R)-1-acryloyl-5- (methoxymethyl)pyrrolidin-3-yl)-3- ((6-fluoro-1-isopropyl-1H- benzo[d]imidazol-5-yl)ethynyl)-5- (methylamino)-1H-pyrazole-4- carboxamide |
| 238 | 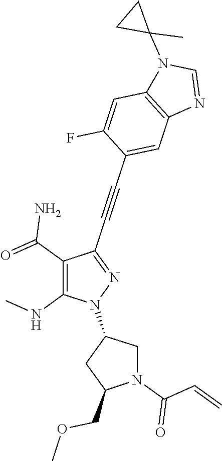 [SMILES: C=CC(=O)N1C[C@@H](n2nc(C#Cc3cc4ncn(C5(C)CC5)c4cc3F)c(C(N)=O)c2NC)C[C@@H]1COC] | 3-{2-[6-fluoro-1-(1- methylcyclopropyl)-1,3-benzodiazol-5- yl]ethynyl}-1-[(3S,5R)-5- (methoxymethyl)-1-(prop-2- enoyl)pyrrolidin-3-yl]-5- (methylamino)pyrazole-4-carboxamide |
| 239 | 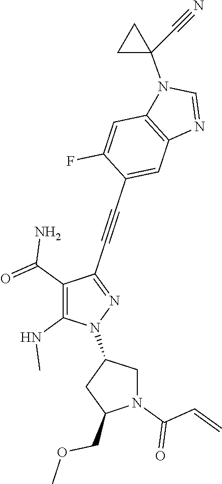 [SMILES: C=CC(=O)N1C[C@@H](n2nc(C#Cc3cc4ncn(C5(C#N)CC5)c4cc3F)c(C(N)=O)c2NC)C[C@@H]1COC] | 1-((3S,5R)-1-acryloyl-5- (methoxymethyl)pyrrolidin-3-yl)-3- ((1-(1-cyanocyclopropyl)-6-fluoro-1H- benzo[d]imidazol-5-yl)ethynyl)-5- (methylamino)-1H-pyrazole-4- carboxamide |
| 240 | 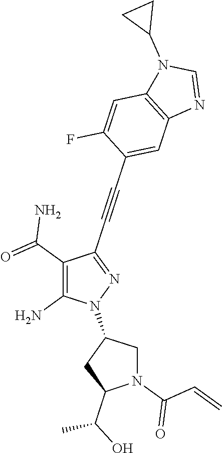 [SMILES: C=CC(=O)N1C[C@@H](n2nc(C#Cc3cc4ncn(C5CC5)c4cc3F)c(C(N)=O)c2N)C[C@@H]1[C@@H](C)O] | 5-amino-3-[2-(1-cyclopropyl-6-fluoro- 1,3-benzodiazol-5-yl)ethynyl]-1- [(3S,5R)-5-[(1R)-1-hydroxyethyl]-1- (prop-2-enoyl)pyrrolidin-3- yl]pyrazole-4-carboxamide |
| 241 | 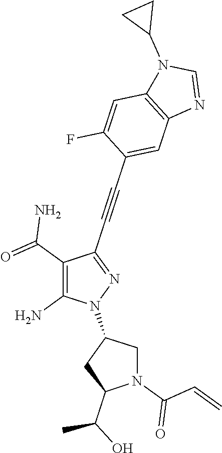 [SMILES: C=CC(=O)N1C[C@@H](n2nc(C#Cc3cc4ncn(C5CC5)c4cc3F)c(C(N)=O)c2N)C[C@@H]1[C@H](C)O] | 5-amino-3-[2-(1-cyclopropyl-6-fluoro- 1,3-benzodiazol-5-yl)ethynyl]-1- [(3S,5R)-5-[(1S)-1-hydroxyethyl]-1- (prop-2-enoyl)pyrrolidin-3- yl]pyrazole-4-carboxamide |
| 242 | 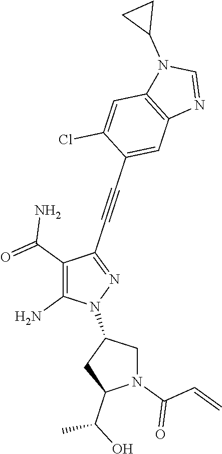 [SMILES: C=CC(=O)N1C[C@@H](n2nc(C#Cc3cc4ncn(C5CC5)c4cc3Cl)c(C(N)=O)c2N)C[C@@H]1[C@@H](C)O] | 5-amino-3-[2-(6-chloro-1-cyclopropyl- 1,3-benzodiazol-5-yl)ethynyl]-1- [(3S,5R)-5-[(1R)-1-hydroxyethyl]-1- (prop-2-enoyl)pyrrolidin-3- yl]pyrazole-4-carboxamide |
| 243 | 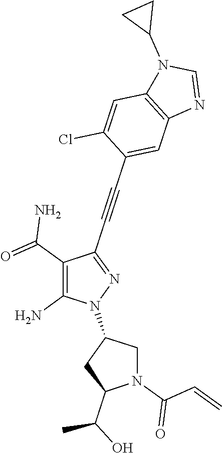 [SMILES: C=CC(=O)N1C[C@@H](n2nc(C#Cc3cc4ncn(C5CC5)c4cc3Cl)c(C(N)=O)c2N)C[C@@H]1[C@H](C)O] | 5-amino-3-[2-(6-chloro-1-cyclopropyl- 1,3-benzodiazol-5-yl)ethynyl]-1- [(3S,5R)-5-[(1S)-1-hydroxyethyl]-1- (prop-2-enoyl)pyrrolidin-3- yl]pyrazole-4-carboxamide |
| 244 | 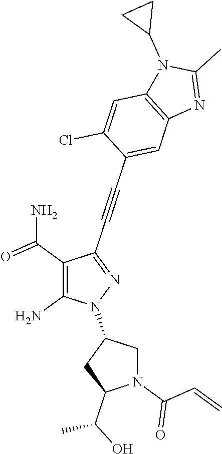 [SMILES: C=CC(=O)N1C[C@@H](n2nc(C#Cc3cc4nc(C)n(C5CC5)c4cc3Cl)c(C(N)=O)c2N)C[C@@H]1[C@@H](C)O] | 1-((3S,5R)-1-acryloyl-5-((R)-1- hydroxyethyl)pyrrolidin-3-yl)-5- amino-3-((6-chloro-1-cyclopropyl-2- methyl-1H-benzo[d]imidazol-5- yl)ethynyl)-1H-pyrazole-4- carboxamide |
| 245 | 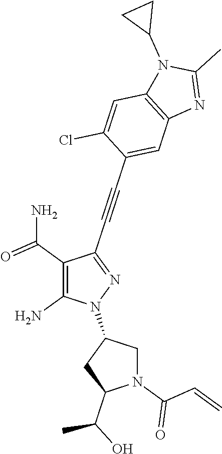 [SMILES: C=CC(=O)N1C[C@@H](n2nc(C#Cc3cc4nc(C)n(C5CC5)c4cc3Cl)c(C(N)=O)c2N)C[C@@H]1[C@H](C)O] | 1-((3S,5R)-1-Acryloyl-5-((S)-1- hydroxyethyl)pyrrolidin-3-yl)-5- amino-3-((6-chloro-1-cyclopropyl-2- methyl-1H-benzo[d]imidazol-5- yl)ethynyl)-1H-pyrazole-4- carboxamide |
| 246 | 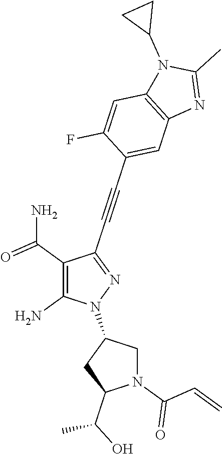 [SMILES: C=CC(=O)N1C[C@@H](n2nc(C#Cc3cc4nc(C)n(C5CC5)c4cc3F)c(C(N)=O)c2N)C[C@@H]1[C@@H](C)O] | 5-amino-3-[2-(1-cyclopropyl-6-fluoro- 2-methyl-1,3-benzodiazol-5- yl)ethynyl]-1-[(3S,5R)-5-[(1R)-1- hydroxyethyl]-1-(prop-2- enoyl)pyrrolidin-3-yl]pyrazole-4- carboxamide |
| 247 | 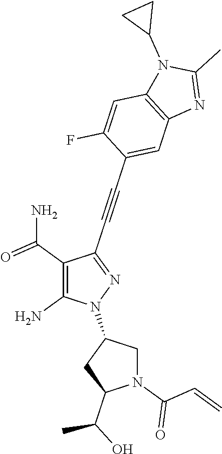 [SMILES: C=CC(=O)N1C[C@@H](n2nc(C#Cc3cc4nc(C)n(C5CC5)c4cc3F)c(C(N)=O)c2N)C[C@@H]1[C@H](C)O] | 5-amino-3-[2-(1-cyclopropyl-6-fluoro- 2-methyl-1,3-benzodiazol-5- yl)ethynyl]-1-[(3S,5R)-5-[(1S)-1- hydroxyethyl]-1-(prop-2- enoyl)pyrrolidin-3-yl]pyrazole-4- carboxamide |
| 248 | 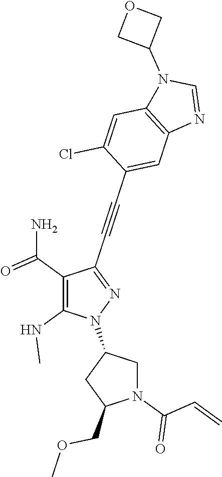 [SMILES: C=CC(=O)N1C[C@@H](n2nc(C#Cc3cc4ncn(C5COC5)c4cc3Cl)c(C(N)=O)c2NC)C[C@@H]1COC] | 3-{2-[6-chloro-1-(oxetan-3-yl)-1,3- benzodiazol-5-yl]ethynyl}-1-[3S,5R)- 5-(methoxymethyl)-1-(prop-2- enoyl)pyrrolidin-3-yl]-5- (methylamino)pyrazole-4-carboxamide |
| 249 | 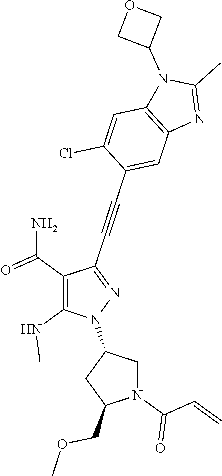 [SMILES: C=CC(=O)N1C[C@@H](n2nc(C#Cc3cc4nc(C)n(C5COC5)c4cc3Cl)c(C(N)=O)c2NC)C[C@@H]1COC] | 3-{2-[6-chloro-2-methyl-1-(oxetan-3- yl)-1,3-benzodiazol-5-yl]ethynyl}-1- [(3S,5R)-5-(methoxymethyl)-1-(prop- 2-enoyl)pyrrolidin-3-yl]-5- (methylamino)pyrazole-4-carboxamide |
| 250 | 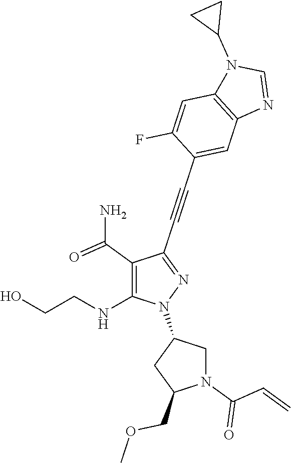 [SMILES: C=CC(=O)N1C[C@@H](n2nc(C#Cc3cc4ncn(C5CC5)c4cc3F)c(C(N)=O)c2NCCO)C[C@@H]1COC] | 3-[2-(1-cyclopropyl-6-fluoro-1,3- benzodiazol-5-yl)ethynyl]-5-[(2- hydroxyethyl)amino]-1-[(3S,5R)-5- (methoxymethyl)-1-(prop-2- enoyl)pyrrolidin-3-yl]pyrazole-4- carboxamide |
| 251 | 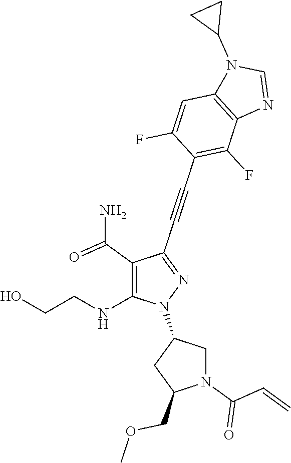 [SMILES: C=CC(=O)N1C[C@@H](n2nc(C#Cc3c(F)cc4c(ncn4C4CC4)c3F)c(C(N)=O)c2NCCO)C[C@@H]1COC] | 3-[2-(1-cyclopropyl-4,6-difluoro-1,3- benzodiazol-5-yl)ethynyl]-5-[(2- hydroxyethyl)amino]-1-[(3S,5R)-5- (methoxymethyl)-1-(prop-2- enoyl)pyrrolidin-3-yl]pyrazole-4- carboxamide |
| 252 | 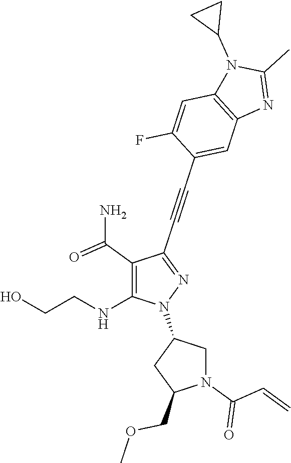 [SMILES: C=CC(=O)N1C[C@@H](n2nc(C#Cc3cc4nc(C)n(C5CC5)c4cc3F)c(C(N)=O)c2NCCO)C[C@@H]1COC] | 3-[2-(1-cyclopropyl-6-fluoro-2- methyl-1,3-benzodiazol-5-yl)ethynyl]- 5-[(2-hydroxyethyl)amino]-1-[(3S,5R)- 5-(methoxymethyl)-1-(prop-2- enoyl)pyrrolidin-3-yl]pyrazole-4- carboxamide |
| 253 | 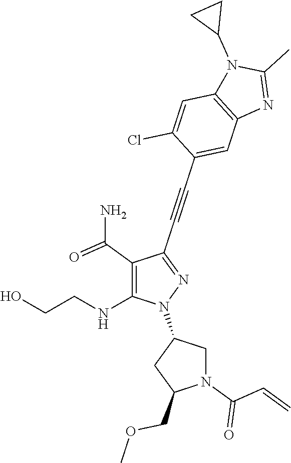 [SMILES: C=CC(=O)N1C[C@@H](n2nc(C#Cc3cc4nc(C)n(C5CC5)c4cc3Cl)c(C(N)=O)c2NCCO)C[C@@H]1COC] | 1-((3S,5R)-1-acryloyl-5- (methoxymethyl)pyrrolidin-3-yl)-3- ((6-chloro-1-cyclopropyl-2-methyl- 1H-benzo[d]imidazol-5-yl)ethynyl)-5- ((2-hydroxyethyl)amino)-1H-pyrazole- 4-carboxamide |
| 254 | 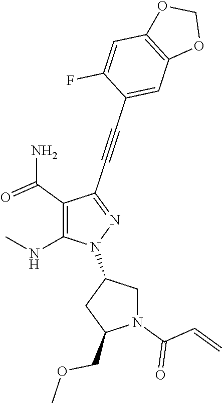 [SMILES: C=CC(=O)N1C[C@@H](n2nc(C#Cc3cc4c(cc3F)OCO4)c(C(N)=O)c2NC)C[C@@H]1COC] | 3-[2-(6-fluoro-2H-1,3-benzodioxol-5- yl)ethynyl]-1-[(3S,5R)-5- (methoxymethyl)-1-(prop-2- enoyl)pyrrolidin-3-yl]-5- (methylamino)pyrazole-4-carboxamide |
| 256 | 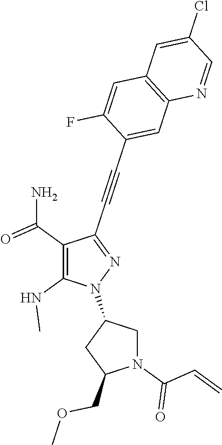 [SMILES: C=CC(=O)N1C[C@@H](n2nc(C#Cc3cc4ncc(Cl)cc4cc3F)c(C(N)=O)c2NC)C[C@@H]1COC] | 3-[2-(3-chloro-6-fluoroquinolin-7- yl)ethynyl]-1-[(3S,5R)-5- (methoxymethyl)-1-(prop-2- enoyl)pyrrolidin-3-yl]-5- (methylamino)pyrazole-4-carboxamide |
| 257 | 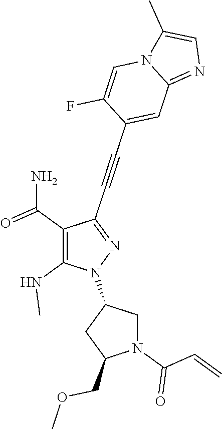 [SMILES: C=CC(=O)N1C[C@@H](n2nc(C#Cc3cc4ncc(C)n4cc3F)c(C(N)=O)c2NC)C[C@@H]1COC] | 1-((3S,5R)-1-acryloyl-5- (methoxymethyl)pyrrolidin-3-yl)-3- ((6-fluoro-3-methylimidazo[1,2- a]pyridin-7-yl)ethynyl)-5- (methylamino)-1H-pyrazole-4- carboxamide |
| 258 | 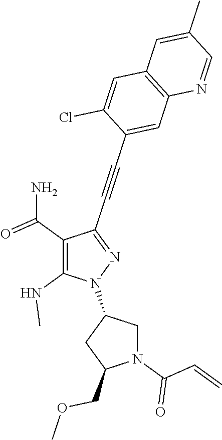 [SMILES: C=CC(=O)N1C[C@@H](n2nc(C#Cc3cc4ncc(C)cc4cc3Cl)c(C(N)=O)c2NC)C[C@@H]1COC] | 1-((3S,5R)-1-acryloyl-5- (methoxymethyl)pyrrolidin-3-yl)-3- ((6-chloro-3-methylquinolin-7- yl)ethynyl)-5-(methylamino)-1H- pyrazole-4-carboxamide |
| 259 | 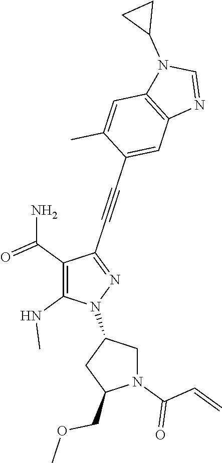 [SMILES: C=CC(=O)N1C[C@@H](n2nc(C#Cc3cc4ncn(C5CC5)c4cc3C)c(C(N)=O)c2NC)C[C@@H]1COC] | 3-[2-(1-cyclopropyl-6-methyl-1,3- benzodiazol-5-yl)ethynyl]-1-[(3S,5R)- 5-(methoxymethyl)-1-(prop-2- enoyl)pyrrolidin-3-yl]-5- (methylamino)pyrazole-4-carboxamide |
| 260 | 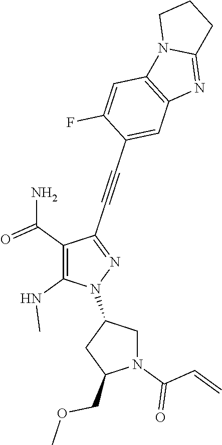 [SMILES: C=CC(=O)N1C[C@@H](n2nc(C#Cc3cc4nc5n(c4cc3F)CCC5)c(C(N)=O)c2NC)C[C@@H]1COC] | 1-((3S,5R)-1-acryloyl-5- (methoxymethyl)pyrrolidin-3-yl)-3- ((7-fluoro-2,3-dihydro-1H- benzo[d]pyrrolo[1,2-a]imidazol-6- yl)ethynyl)-5-(methylamino)-1H- pyrazole-4-carboxamide |
| 261 | 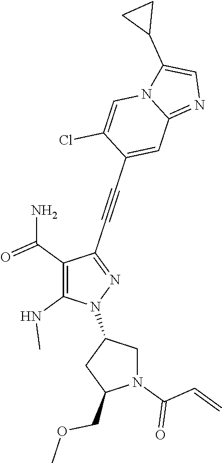 [SMILES: C=CC(=O)N1C[C@@H](n2nc(C#Cc3cc4ncc(C5CC5)n4cc3Cl)c(C(N)=O)c2NC)C[C@@H]1COC] | 3-(2-{6-chloro-3- cyclopropylimidazo[1,2-a]pyridin-7- yl}ethynyl)-1-[(3S,5R)-5- (methoxymethyl)-1-(prop-2- enoyl)pyrrolidin-3-yl]-5- (methylamino)pyrazole-4-carboxamide |
| 262 | 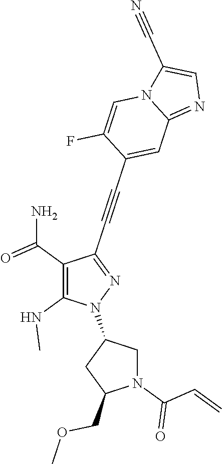 [SMILES: C=CC(=O)N1C[C@@H](n2nc(C#Cc3cc4ncc(C#N)n4cc3F)c(C(N)=O)c2NC)C[C@@H]1COC] | 3-(2-{3-cyano-6-fluoroimidazo[1,2- a]pyridin-7-yl}ethynyl)-1-[(3S,5R)-5- (methoxymethyl)-1-(prop-2- enoyl)pyrrolidin-3-yl]-5- (methylamino)pyrazole-4-carboxamide |
| 263 | 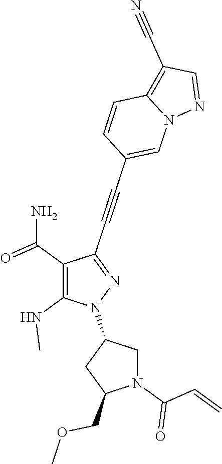 [SMILES: C=CC(=O)N1C[C@@H](n2nc(C#Cc3ccc4c(C#N)cnn4c3)c(C(N)=O)c2NC)C[C@@H]1COC] | 3-(2-{3-cyanopyrazolo[1,5-a]pyridin- 6-yl}ethynyl)-1-[(3S,5R)-5- (methoxymethyl)-1-(prop-2- enoyl)pyrrolidin-3-yl]-5- (methylamino)pyrazole-4-carboxamide |
| 264 | 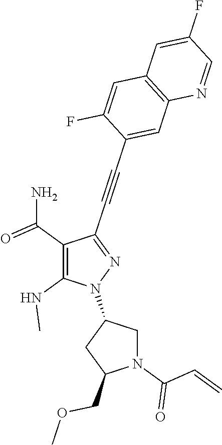 [SMILES: C=CC(=O)N1C[C@@H](n2nc(C#Cc3cc4ncc(F)cc4cc3F)c(C(N)=O)c2NC)C[C@@H]1COC] | 3-[2-(3,6-difluoroquinolin-7- yl)ethynyl]-1-[(3S,5R)-5- (methoxymethyl)-1-(prop-2- enoyl)pyrrolidin-3-yl]-5- (methylamino)pyrazole-4-carboxamide |
| 265 | 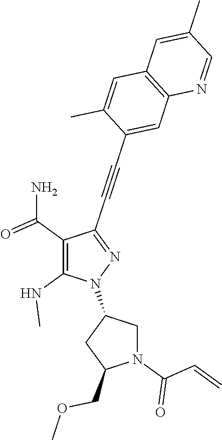 [SMILES: C=CC(=O)N1C[C@@H](n2nc(C#Cc3cc4ncc(C)cc4cc3C)c(C(N)=O)c2NC)C[C@@H]1COC] | 3-[2-(3,6-difluoroquinolin-7- yl)ethynyl]-1-[(3S,5R)-5- (methoxymethyl)-1-(prop-2- enoyl)pyrrolidin-3-yl]-5- (methylamino)pyrazole-4-carboxamide |
| 266 | 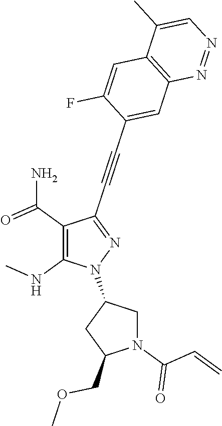 [SMILES: C=CC(=O)N1C[C@@H](n2nc(C#Cc3cc4nncc(C)c4cc3F)c(C(N)=O)c2NC)C[C@@H]1COC] | 1-((3S,5R)-1-acryloyl-5- (methoxymethyl)pyrrolidin-3-yl)-3- ((6-fluoro-4-methylcinnolin-7- yl)ethynyl)-5-(methylamino)-1H- pyrazole-4-carboxamide |
| 267 | 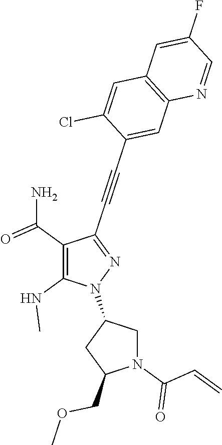 [SMILES: C=CC(=O)N1C[C@@H](n2nc(C#Cc3cc4ncc(F)cc4cc3Cl)c(C(N)=O)c2NC)C[C@@H]1COC] | 3-[2-(6-chloro-3-fluoroquinolin-7- yl)ethynyl]-1-[(3S,5R)-5- (methoxymethyl)-1-(prop-2- enoyl)pyrrolidin-3-yl]-5- (methylamino)pyrazole-4-carboxamide |
| 268 | 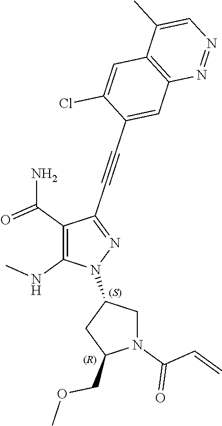 | 1-((3S,5R)-1-acryloyl-5- (methoxymethyl)pyrrolidin-3-yl)-3- ((6-chloro-4-methylcinnolin-7- yl)ethynyl)-5-(methylamino)-1H- pyrazole-4-carboxamide |
| 269 | 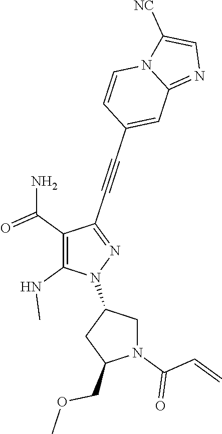 [SMILES: C=CC(=O)N1C[C@@H](n2nc(C#Cc3ccn4c(C#N)cnc4c3)c(C(N)=O)c2NC)C[C@@H]1COC] | 3-(2-{3-cyanoimidazo[1,2-a]pyridin-7- yl}ethynyl)-1-[(3S,5R)-5- (methoxymethyl)-1-(prop-2- enoyl)pyrrolidin-3-yl]-5- (methylamino)pyrazole-4-carboxamide |
| 270 | 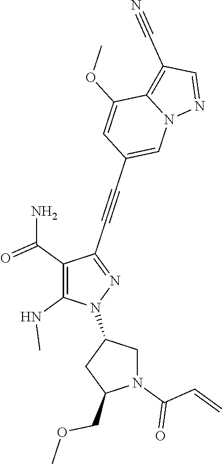 [SMILES: C=CC(=O)N1C[C@@H](n2nc(C#Cc3cc(OC)c4c(C#N)cnn4c3)c(C(N)=O)c2NC)C[C@@H]1COC] | 1-((3S,5R)-1-acryloyl-5- (methoxymethyl)pyrrolidin-3-yl)-3- ((3-cyano-4-methoxypyrazolo[1,5- a]pyridin-6-yl)ethynyl)-5- (methylamino)-1H-pyrazole-4- carboxamide |
| 271 | 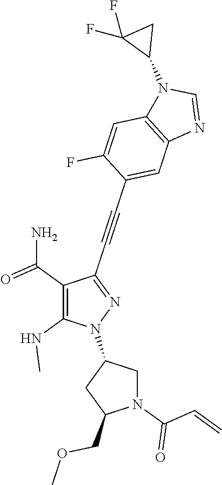 [SMILES: C=CC(=O)N1C[C@@H](n2nc(C#Cc3cc4ncn([C@H]5CC5(F)F)c4cc3F)c(C(N)=O)c2NC)C[C@@H]1COC] | 3-(2-{1-[(1S)-2,2- Difluorocyclopropyl]-6-fluoro-1,3- benzodiazol-5-yl}ethynyl)-1-[(3S,5R)- 5-(methoxymethyl)-1-(prop-2- enoyl)pyrrolidin-3-yl]-5- (methylamino)pyrazole-4-carboxamide |
| 272 | 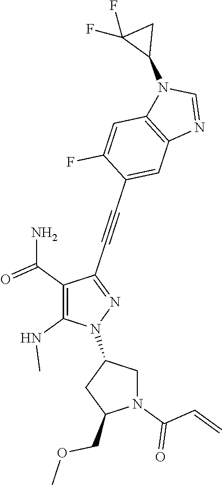 [SMILES: C=CC(=O)N1C[C@@H](n2nc(C#Cc3cc4ncn([C@@H]5CC5(F)F)c4cc3F)c(C(N)=O)c2NC)C[C@@H]1COC] | 3-(2-{1-[((R)-2,2- difluorocyclopropyl)-6-fluoro-1,3- benzodiazol-5-yl]ethynyl}-1-[(3S,5R)- 5-(methoxymethyl)-1-(prop-2- enoyl)pyrrolidin-3-yl]-5- (methylamino)pyrazole-4-carboxamide |
| 273 | 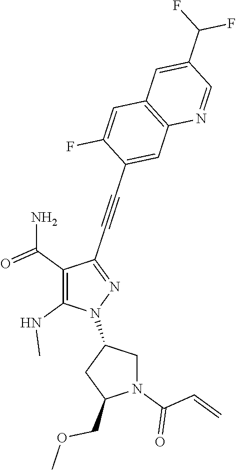 [SMILES: C=CC(=O)N1C[C@@H](n2nc(C#Cc3cc4ncc(C(F)F)cc4cc3F)c(C(N)=O)c2NC)C[C@@H]1COC] | 3-{2-[3-(difluoromethyl)-6- fluoroquinolin-7-yl]ethynyl}-1- [(3S,5R)-5-(methoxymethyl)-1-(prop- 2-enoyl)pyrrolidin-3-yl]-5- (methylamino)pyrazole-4-carboxamide |
| 274 | 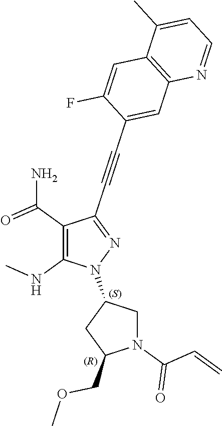 | 3-[2-(6-fluoro-4-methylquinolin-7- yl)ethynyl]-1-[(3S,5R)-5- (methoxymethyl)-1-(prop-2- enoyl)pyrrolidin-3-yl]-5- (methylamino)pyrazole-4-carboxamide |
| 275 | 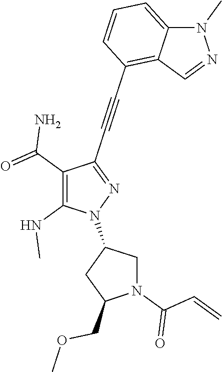 [SMILES: C=CC(=O)N1C[C@@H](n2nc(C#Cc3cccc4c3cnn4C)c(C(N)=O)c2NC)C[C@@H]1COC] | 1-((3S,5R)-1-acryloyl-5- (methoxymethyl)pyrrolidin-3-yl)-3- ((1-methyl-1H-indazol-4-yl)ethynyl)- 5-(methylamino)-1H-pyrazole-4- carboxamide |
| 276 | 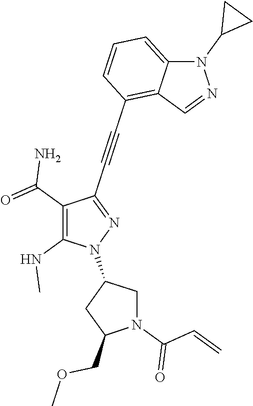 [SMILES: C=CC(=O)N1C[C@@H](n2nc(C#Cc3cccc4c3cnn4C3CC3)c(C(N)=O)c2NC)C[C@@H]1COC] | 3-[2-(1-cyclopropylindazol-4- yl)ethynyl]-1-[(3S,5R)-5- (methoxymethyl)-1-(prop-2- enoyl)pyrrolidin-3-yl]-5- (methylamino)pyrazole-4-carboxamide |
| 277 | 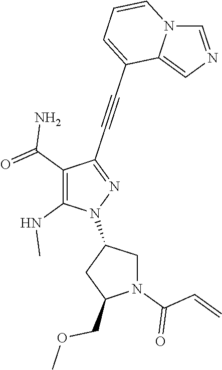 [SMILES: C=CC(=O)N1C[C@@H](n2nc(C#Cc3cccn4cncc34)c(C(N)=O)c2NC)C[C@@H]1COC] | 3-(2-{imidazo[1,5-a]pyridin-8- yl}ethynyl)-1-[(3S,5R)-5- (methoxymethyl)-1-(prop-2- enoyl)pyrrolidin-3-yl]-5- (methylamino)pyrazole-4-carboxamide |
| 278 | 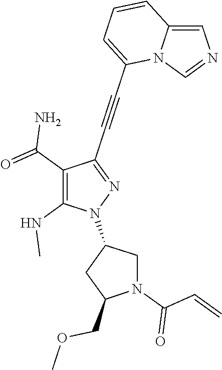 [SMILES: C=CC(=O)N1C[C@@H](n2nc(C#Cc3cccc4cncn34)c(C(N)=O)c2NC)C[C@@H]1COC] | 3-(2-{imidazo[1,5-a]pyridin-5- yl}ethynyl)-1-[(3S,5R)-5- (methoxymethyl)-1-(prop-2- enoyl)pyrrolidin-3-yl]-5- (methylamino)pyrazole-4-carboxamide |
| 279 | 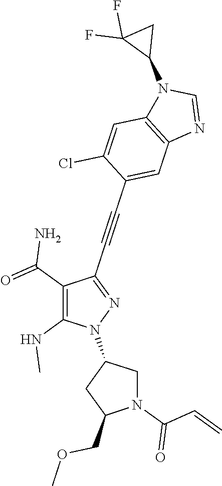 [SMILES: C=CC(=O)N1C[C@@H](n2nc(C#Cc3cc4ncn([C@@H]5CC5(F)F)c4cc3Cl)c(C(N)=O)c2NC)C[C@@H]1COC] | 3-(2-{6-chloro-1-[(1R)-2,2- difluorocyclopropyl]-1,3-benzodiazol- 5-yl}ethynyl)-1-[(3S,5R)-5- (methoxymethyl)-1-(prop-2- enoyl)pyrrolidin-3-yl]-5- (methylamino)pyrazole-4-carboxamide |
| 280 | 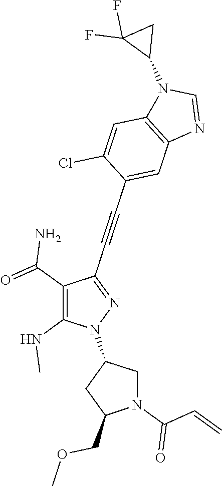 [SMILES: C=CC(=O)N1C[C@@H](n2nc(C#Cc3cc4ncn([C@H]5CC5(F)F)c4cc3Cl)c(C(N)=O)c2NC)C[C@@H]1COC] | 3-(2-{6-Chloro-1-[(1S)-2,2- difluorocyclopropyl]-1,3-benzodiazol- 5-yl}ethynyl)-1-[(3S,5R)-5- (methoxymethyl)-1-(prop-2- enoyl)pyrrolidin-3-yl]-5- (methylamino)pyrazole-4-carboxamide |
| 281 | 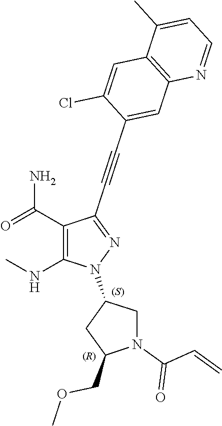 | 3-[2-(6-chloro-4-methylquinolin-7- yl)ethynyl]-1-[(3S,5R)-5- (methoxymethyl)-1-(prop-2- enoyl)pyrrolidin-3-yl]-5- (methylamino)pyrazole-4-carboxamide |
| 282 | 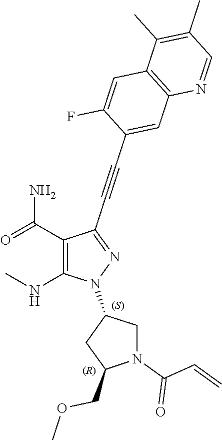 | 1-((3S,5R)-1-acryloyl-5- (methoxymethyl)pyrrolidin-3-yl)-3- ((6-fluoro-3,4-dimethylquinolin-7- yl)ethynyl)-5-(methylamino)-1H- pyrazole-4-carboxamide |
| 283 | 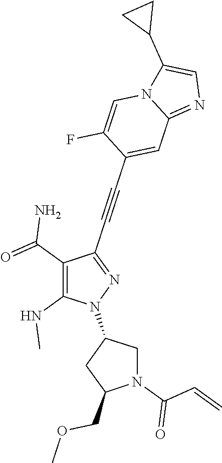 [SMILES: C=CC(=O)N1C[C@@H](n2nc(C#Cc3cc4ncc(C5CC5)n4cc3F)c(C(N)=O)c2NC)C[C@@H]1COC] | 3-(2-{3-cyclopropyl-6- fluoroimidazo[1,2-a]pyridin-7- yl}ethynyl)-1-[(3S,5R)-5- (methoxymethyl)-1-(prop-2- enoyl)pyrrolidin-3-yl]-5- (methylamino)pyrazole-4-carboxamide |
| 284 | 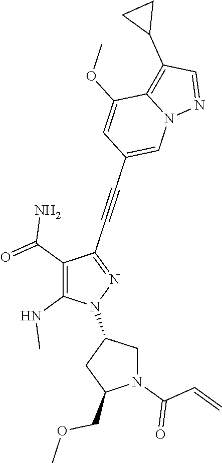 [SMILES: C=CC(=O)N1C[C@@H](n2nc(C#Cc3cc(OC)c4c(C5CC5)cnn4c3)c(C(N)=O)c2NC)C[C@@H]1COC] | 3-(2-{3-cyclopropyl-4- methoxypyrazolo[1,5-a]pyridin-6- yl}ethynyl)-1-[(3S,5R)-5- (methoxymethyl)-1-(prop-2- enoyl)pyrrolidin-3-yl]-5- (methylamino)pyrazole-4-carboxamide |
| 285 | 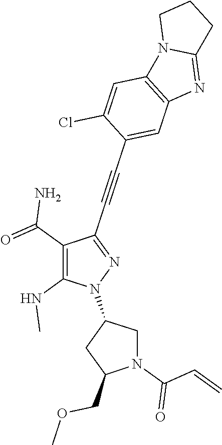 [SMILES: C=CC(=O)N1C[C@@H](n2nc(C#Cc3cc4nc5n(c4cc3Cl)CCC5)c(C(N)=O)c2NC)C[C@@H]1COC] | 1-((3S,5R)-1-acryloyl-5- (methoxymethyl)pyrrolidin-3-yl)-3- ((7-chloro-2,3-dihydro-1H- benzo[d]pyrrolo[1,2-a]imidazol-6- yl)ethynyl)-5-(methylamino)-1H- pyrazole-4-carboxamide |
| 287 | 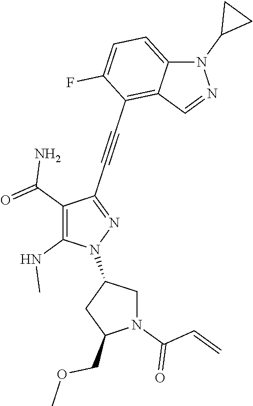 [SMILES: C=CC(=O)N1C[C@@H](n2nc(C#Cc3c(F)ccc4c3cnn4C3CC3)c(C(N)=O)c2NC)C[C@@H]1COC] | 1-((3S,5R)-1-acryloyl-5- (methoxymethyl)pyrrolidin-3-yl)-3- ((1-cyclopropyl-6-fluoro-1H-indazol- 4-yl)ethynyl)-5-(methylamino)-1H- pyrazole-4-carboxamide |
| 288 | 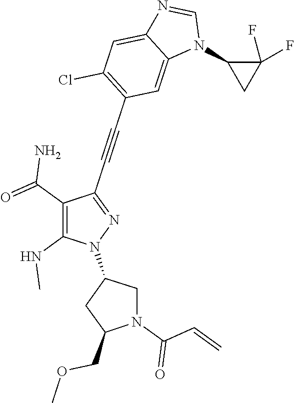 [SMILES: C=CC(=O)N1C[C@@H](n2nc(C#Cc3cc4c(cc3Cl)ncn4[C@@H]3CC3(F)F)c(C(N)=O)c2NC)C[C@@H]1COC] | 3-(2-{6-chloro-3-[(1R)-2,2- difluorocyclopropyl]-1,3-benzodiazol- 5-yl}ethynyl)-1-[(3S,5R)-5- (methoxymethyl)-1-(prop-2- enoyl)pyrrolidin-3-yl]-5- (methylamino)pyrazole-4-carboxamide |
| 289 | 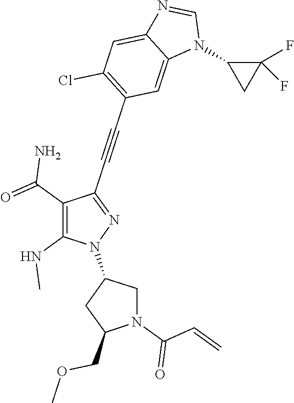 [SMILES: C=CC(=O)N1C[C@@H](n2nc(C#Cc3cc4c(cc3Cl)ncn4[C@H]3CC3(F)F)c(C(N)=O)c2NC)C[C@@H]1COC] | 3-(2-{6-chloro-3-[(1S)-2,2- difluorocyclopropyl]-1,3-benzodiazol- 5-yl}ethynyl)-1-[(3S,5R)-5- (methoxymethyl)-1-(prop-2- enoyl)pyrrolidin-3-yl]-5- (methylamino)pyrazole-4-carboxamide |
| 290 | 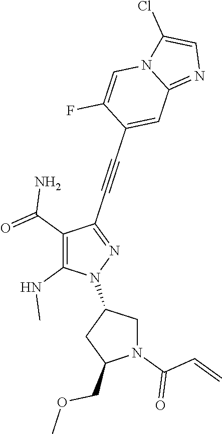 [SMILES: C=CC(=O)N1C[C@@H](n2nc(C#Cc3cc4ncc(Cl)n4cc3F)c(C(N)=O)c2NC)C[C@@H]1COC] | 3-(2-{3-chloro-6-fluoroimidazo[1,2- a]pyridin-7-yl}ethynyl)-1-[(3S,5R)-5- (methoxymethyl)-1-(prop-2- enoyl)pyrrolidin-3-yl]-5- (methylamino)pyrazole-4-carboxamide |

In some embodiments, the heteroaromatic FGFR kinase inhibitory compound disclosed herein has a structure provided in Table 2.

TABLE 2

| | |
|---|---|
| 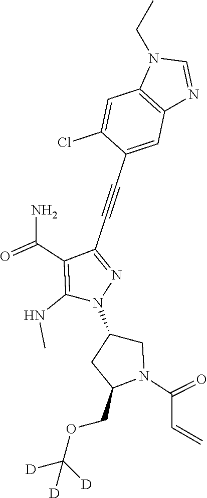 [SMILES: [2H]C([2H])([2H])OC[C@H]1C[C@H](n2nc(C#Cc3cc4ncn(CC)c4cc3Cl)c(C(N)=O)c2NC)CN1C(=O)C=C] | 1-((3S,5R)-1-acryloyl-5- ((methoxy- d3)methyl)pyrrolidin-3-yl)- 3-((6-chloro-1-ethyl-1H benzo[d]imidazol-5- yl)ethynyl)-5- (methylamino)-1H-pyrazole- 4-carboxamide |
| 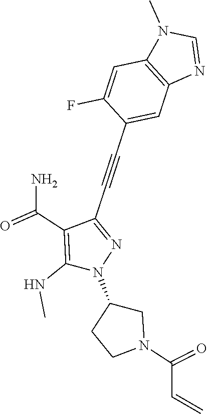 [SMILES: C=CC(=O)N1CC[C@H](n2nc(C#Cc3cc4ncn(C)c4cc3F)c(C(N)=O)c2NC)C1] | (S)-1-(1-acryloylpyrrolidin- 3-yl)-3-((6-fluoro-1-methyl- 1H-benzo[d]imidazol-5- yl)ethynyl)-5- (methylamino)-1H-pyrazole- 4-carboxamide |
| 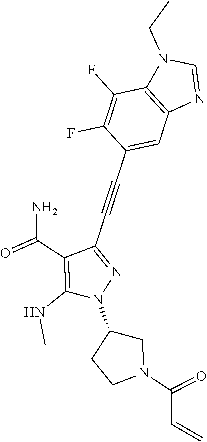 [SMILES: C=CC(=O)N1CC[C@H](n2nc(C#Cc3cc4ncn(CC)c4c(F)c3F)c(C(N)=O)c2NC)C1] | (S)-1-(1-acryloylpyrrolidin- 3-yl)-3-((1-ethyl-6,7- difluoro-1H- benzo[d]imidazol-5- yl)ethynyl)-5- (methylamino)-1H-pyrazole- 4-carboxamide |
| 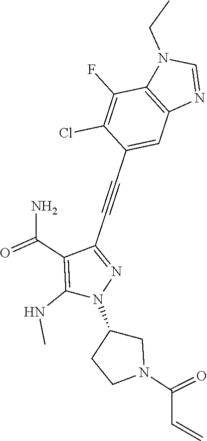 [SMILES: C=CC(=O)N1CC[C@H](n2nc(C#Cc3cc4ncn(CC)c4c(F)c3Cl)c(C(N)=O)c2NC)C1] | (S)-1-(1-acryloylpyrrolidin- 3-yl)-3-((6-chloro-1-ethyl-7- fluoro-1H-benzo[d]imidazol- 5-yl)ethynyl)-5- (methylamino)-1H-pyrazole- 4-carboxamide |
| 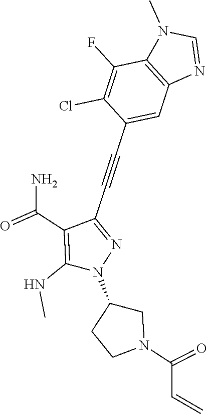 [SMILES: C=CC(=O)N1CC[C@H](n2nc(C#Cc3cc4ncn(C)c4c(F)c3Cl)c(C(N)=O)c2NC)C1] | (S)-1-(1-acryloylpyrrolidin-3- 3-yl)-3-((6-chloro-7-fluoro- 1-methyl-1H- benzo[d]imidazol-5- yl)ethynyl)-5- (methylamino)-1H-pyrazole- 4-carboxamide |
| 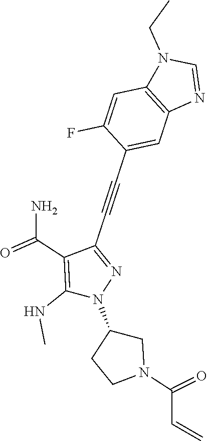 [SMILES: C=CC(=O)N1CC[C@H](n2nc(C#Cc3cc4ncn(CC)c4cc3F)c(C(N)=O)c2NC)C1] | (S)-1-(1-acryloylpyrrolidin- 3-yl)-3-((1-ethyl-6-flouro- 1H-benzo[d]imidazol-5- yl)ethynyl)-5- (methylamino)-1H-pyrazole- 4-carboxamide |
| 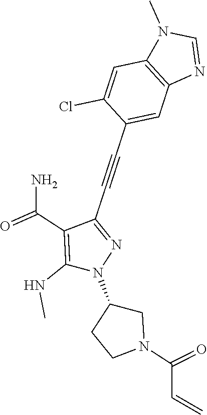 [SMILES: C=CC(=O)N1CC[C@H](n2nc(C#Cc3cc4ncn(C)c4cc3Cl)c(C(N)=O)c2NC)C1] | (S)-1-(1-acryloylpyrrolidin- 3-yl)-3-((6-chloro-1-methyl- 1H-benzo[d]imidazol-5- yl)ethynyl)-5- (methylamino)-1H-pyrazole- 4-carboxamide |
| 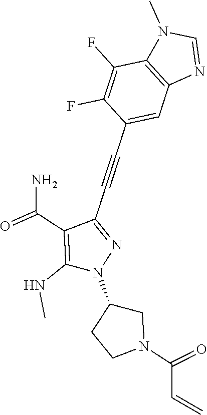 [SMILES: C=CC(=O)N1CC[C@H](n2nc(C#Cc3cc4ncn(C)c4c(F)c3F)c(C(N)=O)c2NC)C1] | (S)-1-(1-acryloylpyrrollidin- 3-yl)-3-((6,7-difluoro-1- methyl-1H- benzo[d]imidazol-5- yl)ethynyl)-5- (methylamino)-1H-pyrazole- 4-carboxamide |
| 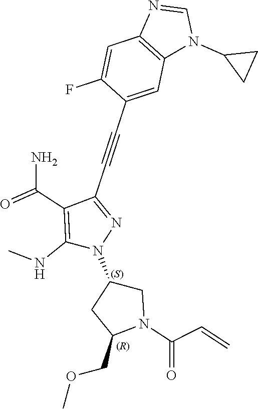 | 1-((3S,5R)-1-acryloyl-5- (methoxymethyl)pyrrolidin- 3-yl)-3-((1-cyclopropyl-5- fluoro-1H-benzo[d]imidazol- 6-yl)ethynyl)-5- (methylamino)-1H-pyrazole- 4-carboxamide |
| 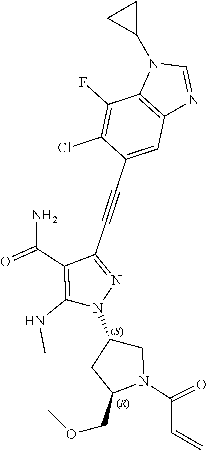 | 1-((3S,5R)-1-acryloyl-5- (methoxymethyl)pyrrolidin- 3-yl)-3-((6-chloro-1- cyclopropyl-7-fluoro-1H- benzo[d]imidazol-5- yl)ethynyl)-5- (methylamino)-1H-pyrazole- 4-carboxamide |
| 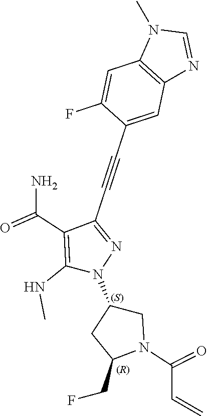 | 1-((3S,5R)-1-acryloyl-5- (fluoromethyl)pyrrolidin-3- yl)-3-((6-fluoro-1-methyl- 1H-benzo[d]imidazol-5- yl)ethynyl)-5- (methylamino)-1H-pyrazole- 4-carboxamide |
| 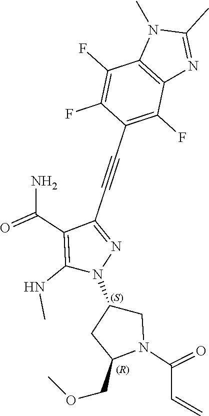 | 1-((3S,5R)-1-acryloyl-5- (methoxymethyl)pyrrolidin- 3-yl)-5-(methylamino)-3- ((4,6,7-trifluoro-1,2- dimethyl-1H- benzo[d]imidazol-5- yl)ethynyl)-1H-pyrazole-4- carboxamide |
| 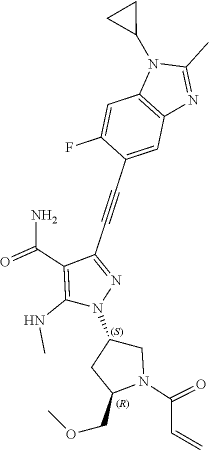 | 1-((3S,5R)-1-acryloyl-5- (methoxymethyl)pyrrolidin- 3-yl)-3-((1-cyclopropyl-6- fluoro-2-methyl-1H- benzo[d]imidazol-5- yl)ethynyl)-5- (methylamino)-1H-pyrazole- 4-carboxamide |
| 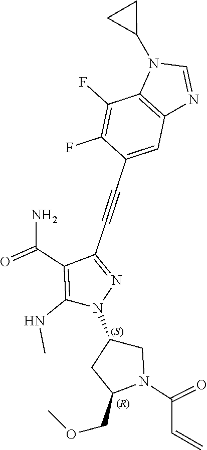 | 1-((3S,5R)-1-acryloyl-5- (methoxymethyl)pyrrolidin- 3-yl)-3-((1-cyclopropyl-6,7- difluoro-1H- benzo[d]imidazol-5- yl)ethynyl)-5- (methylamino)-1H-pyrazole- 4-carboxamide |
| 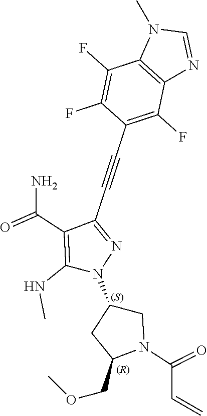 | 1-((3S,5R)-1-acryloyl-5- (methoxymethyl)pyrrolidin- 3-yl)-5-(methylamino)-3- ((4,6,7-trifluoro-1-methyl- 1H-benzo[d]imidazol-5- yl)ethynyl)-1H-pyrazole-4- carboxamide |
| 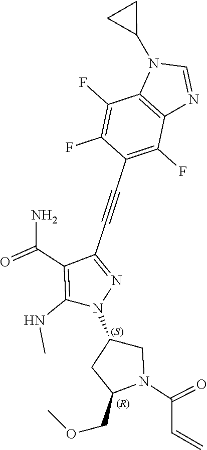 | 1-((3S,5R)-1-acryloyl-5- (methoxymethyl)pyrrolidin- 3-yl-3-((1-cyclopropyl- 4,6,7-trifluoro-1H- benzo[d]imidazol-5- yl)ethynyl)-5- (methylamino)-1H-pyrazole- 4-carboxamide |
| 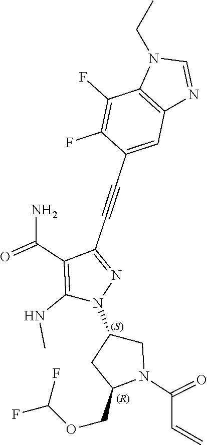 | 1-((3S,5R)-1-acryloyl-5- ((difluoromethoxy)methyl) pyrrolidin-3-yl)-3-((1-ethyl- 6,7-difluoro-1H- benzo[d]imidazol-5- yl)ethnyl)-5- (methylamino)-1H-pyrazole- 4-carboxamide |
| 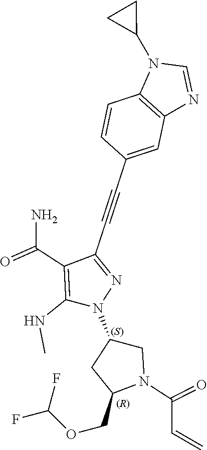 | 1-((3S,5R)-1-acryloyl-5- ((difluoromethoxy)methyl) pyrrolidin-3-yl)-3-((1- cyclopropyl-1H- benzo[d]imidazol-5- yl)ethynyl)-5- (methylamino)-1H-pyrazole- 4-carboxamide |
| 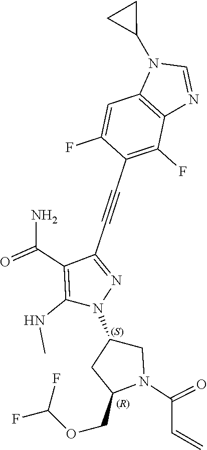 | 1-((3S,5R)-1-acryloyl-5- ((difluoromethoxy)methyl) pyrrolidin-3-yl)-3-((1- cyclopropyl-4,6-difluoro- 1H-benzo[d]imidazol-5- yl)ethynyl)-5- (methylamino)-1H-pyrazole- 4-carboxamide |
| 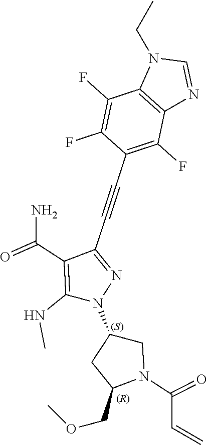 | 1-((3S,5R)-1-acryloyl-5- (methoxymethyl)pyrrolidin- 3-yl)-3-((1-ethyl-4,6,7- trifluoro-1H- benzo[d]imidazol-5- yl)ethynyl)-5- (methylamino)-1H-pyrazole- 4-carboxamide |
| 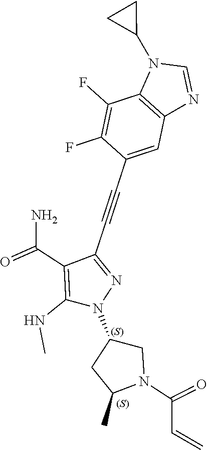 | 1-((3S,5S)-1-acryloyl-5- methylpyrrolidin-3-yl)-3-((1- cyclopropyl-6,7-difluoro- 1H-benzo[d]imidazol-5- yl)ethynyl)-5- (methylamino)-1H-pyrazole- 4-carboxamide |
| 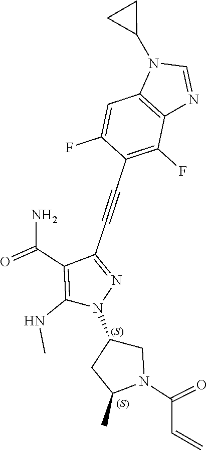 | 1-((3S,5S)-1-acryloyl-5- methylpyrrolidin-3-yl)-3-((1- cyclopropyl-4,6-difluoro- 1H-benzo[d]imidazol-5- yl)ethynyl)-5- (methylamino)-1H-pyrazole- 4-carboxamide |
| 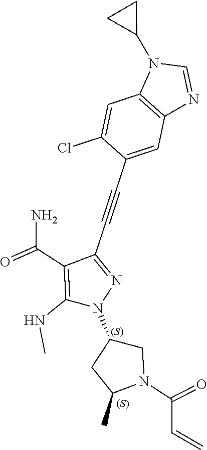 | 1-((3S,5S)-1-acryloyl-5- methylpyrrolidin-3-yl)-3-((6- chloro-1-cyclopropyl-1H- benzo[d]imidazol-5- yl)ethynyl)-5- (methylamino)-1H-pyrazole- 4-carboxamide |
| 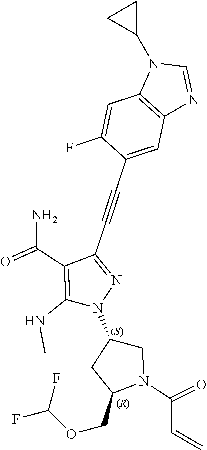 | 1-((3S,5R)-1-acryloyl-5- (difluoromethoxy)methyl) pyrrolidin-3-yl)-3-((1- cyclopropyl-6-fluoro-1H- benzo[d]imidazol-5- yl)ethynyl)-5- (methylamino)-1H-pyrazole- 4-carboxamide |
| 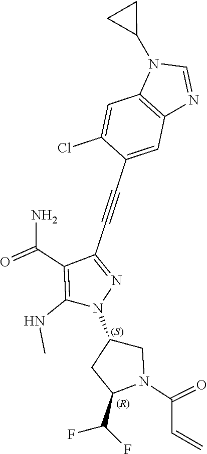 | 1-((3S,5R)-1-acryloyl-5- (difluoromethyl)pyrrolidin- 3-yl)-3-((6-chloro-1- cyclopropyl-1H- benzo[d]imidazol-5- yl)ethynyl)-5- (methylamino)-1H-pyrazole- 4-carboxamide |
| 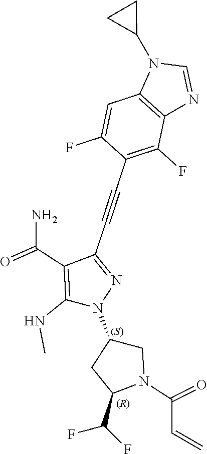 | 1-((3S,5R)-1-acryloyl-5- (difluoromethyl)pyrrolidin- 3-yl)-3-((1-cyclopropyl-4,6- difluoro-1H- benzo[d]imidazol-5- yl)ethynyl)-5- (methylamino)-1H-pyrazole- 4-carboxamide |
| 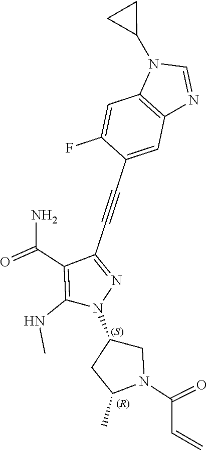 | 1-((3S,5R)-1-acryloyl-5- methylpyrrolidin-3-yl)-3-((1- cyclopropyl-6-fluoro-1H- benzo[d]imidazol-5- yl)ethynyl)-5- (methylamino)-1H-pyrazole- 4-carboxamide |
| 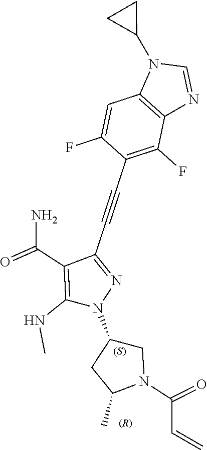 | 1-((3S,5R)-1-acryloyl-5- methylpyrrolidin-3-yl)-3-((1- cyclopropyl-4,6-difluoro- 1H-benzo[d]imidazol-5- yl)ethynyl)-5- (methylamino)-1H-pyrazole- 4-carboxamide |
| 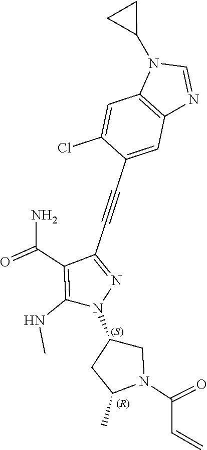 | 1-((3S,5R)-1-acryloyl-5- methylpyrrolidin-3-yl)-3-((6- chloro-1-cyclopropyl-1H- benzo[d]imidazol-5- yl)ethynyl)-5- (methylamino)-1H-pyrazole- 4-carboxamide |

### Preparation of Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line databases (contact the American Chemical Society, Washington, D.C. for more details). Chemicals that are known but not commercially available in catalogs are optionally prepared by custom chemical synthesis houses, where many of the standard chemical supply houses (e.g., those listed above) provide custom synthesis services. A reference useful for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Pharmaceutical Compositions

In certain embodiments, the heteroaromatic FGFR kinase inhibitory compound described herein is administered as a pure chemical. In other embodiments, the heteroaromatic FGFR kinase inhibitory compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Provided herein is a pharmaceutical composition comprising at least one heteroaromatic FGFR kinase inhibitory compound as described herein, or a stereoisomer, pharmaceutically acceptable salt, hydrate, or solvate thereof, together with one or more pharmaceutically acceptable carriers. The carrier(s) (or excipient(s)) is acceptable or suitable if the carrier is compatible with the other ingredients of the composition and not deleterious to the recipient (i.e., the subject or the patient) of the composition.

One embodiment provides a pharmaceutical composition comprising a pharmaceutically acceptable excipient and a compound of Formula (I), or a pharmaceutically acceptable salt or solvate thereof.

One embodiment provides a method of preparing a pharmaceutical composition comprising mixing a compound of Formula (I), or a pharmaceutically acceptable salt or solvate thereof, and a pharmaceutically acceptable carrier.

In certain embodiments, the heteroaromatic FGFR kinase inhibitory compound as described by Formula (I), or a pharmaceutically acceptable salt or solvate thereof, is substantially pure, in that it contains less than about 5%, or less than about 1%, or less than about 0.1%, of other organic small molecules, such as unreacted intermediates or synthesis by-products that are created, for example, in one or more of the steps of a synthesis method.

One embodiment provides a pharmaceutical composition comprising a pharmaceutically acceptable excipient and a compound of Formula (II), or a pharmaceutically acceptable salt or solvate thereof.

One embodiment provides a method of preparing a pharmaceutical composition comprising mixing a compound of Formula (II), or a pharmaceutically acceptable salt or solvate thereof, and a pharmaceutically acceptable carrier.

In certain embodiments, the heteroaromatic FGFR kinase inhibitory compound as described by Formula (II), or a pharmaceutically acceptable salt or solvate thereof, is substantially pure, in that it contains less than about 5%, or less than about 1%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, PA (2005)).

In some embodiments, the heteroaromatic FGFR kinase inhibitory compound as described by Formula (I) or (II), or pharmaceutically acceptable salt or solvate thereof, is formulated for administration by injection. In some instances, the injection formulation is an aqueous formulation. In some instances, the injection formulation is a non-aqueous formulation. In some instances, the injection formulation is an oil-based formulation, such as sesame oil, or the like.

The dose of the composition comprising at least one heteroaromatic FGFR kinase inhibitory compound as described herein differs depending upon the subject or patient's (e.g., human) condition. In some embodiments, such factors include general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

Oral doses typically range from about 1.0 mg to about 1000 mg, one to four times, or more, per day.

### Methods of Treatment

One embodiment provides a compound of Formula (I), or a pharmaceutically acceptable salt or solvate thereof, for use in a method of treatment of the human or animal body.

One embodiment provides a compound of Formula (I), or a pharmaceutically acceptable salt or solvate thereof, for use in a method of treatment of cancer or neoplastic disease.

One embodiment provides a use of a compound of Formula (I), or a pharmaceutically acceptable salt or solvate thereof, in the manufacture of a medicament for the treatment of cancer or neoplastic disease.

In some embodiments, described herein is a method of treating cancer in a patient in need thereof comprising administering to the patient a compound of Formula (I), or a pharmaceutically acceptable salt or solvate thereof. In some embodiments, described herein is a method of treating cancer in a patient in need thereof comprising administering to the patient a pharmaceutical composition comprising a compound of Formula (I), or a pharmaceutically acceptable salt or solvate thereof, and a pharmaceutically acceptable excipient.

One embodiment provides a compound of Formula (II), or a pharmaceutically acceptable salt or solvate thereof, for use in a method of treatment of the human or animal body.

One embodiment provides a compound of Formula (II), or a pharmaceutically acceptable salt or solvate thereof, for use in a method of treatment of cancer or neoplastic disease.

One embodiment provides a use of a compound of Formula (II), or a pharmaceutically acceptable salt or solvate thereof, in the manufacture of a medicament for the treatment of cancer or neoplastic disease.

In some embodiments, described herein is a method of treating cancer in a patient in need thereof comprising administering to the patient a compound of Formula (I), or a pharmaceutically acceptable salt or solvate thereof. In some embodiments, described herein is a method of treating cancer in a patient in need thereof comprising administering to the patient a pharmaceutical composition comprising a compound of Formula (I), or a pharmaceutically acceptable salt or solvate thereof, and a pharmaceutically acceptable excipient.

Provided herein is the method wherein the pharmaceutical composition is administered orally. Provided herein is the method wherein the pharmaceutical composition is administered by injection.

Other embodiments and uses will be apparent to one skilled in the art in light of the present disclosures. The following examples are provided merely as illustrative of various embodiments and shall not be construed to limit the invention in any way.

### EXAMPLES

### I. Chemical Synthesis

In some embodiments, the heteroaromatic FGFR kinase inhibitory compounds disclosed herein are synthesized according to the following examples. As used below, and throughout the description of the invention, the following abbreviations, unless otherwise indicated, shall be understood to have the following meanings:

| | |
|---|---|
| ° C. | degrees Celsius |
| δ_{H} | chemical shift in parts per million downfield from |
| | tetramethylsilane |
| DCM | dichloromethane (CH₂Cl₂) |
| DMF | dimethylformamide |
| DMSO | dimethylsulfoxide |
| EA | ethyl acetate |
| ESI | electrospray ionization |
| Et | ethyl |
| g | gram(s) |
| h | hour(s) |
| HPLC | high performance liquid chromatography |
| Hz | hertz |
| J | coupling constant (in NMR spectrometry) |
| LCMS | liquid chromatography mass spectrometry |
| μ | micro |
| m | multiplet (spectral); meter(s); milli |
| M | molar |
| M⁺ | parent molecular ion |
| Me | methyl |
| MHz | megahertz |
| min | minute(s) |
| mol | mole(s); molecular (as in mol wt) |
| mL | milliliter |
| MS | mass spectrometry |
| nm | nanometer(s) |
| NMR | nuclear magnetic resonance |
| pH | potential of hydrogen; a measure of the acidity or basicity |
| | solution |
| PE | petroleum ether |
| RT | room temperature |
| s | singlet (spectral) |
| t | triplet (spectral) |
| T | temperature |
| TFA | trifluoroacetic acid |
| THF | tetrahydrofuran |

### Intermediate 1: 1-((3S,5R)-1-acryloyl-5-(methoxymethyl)pyrrolidin-3-yl)-3-bromo-5-(methylamino)-1H-pyrazole-4-carboxamide

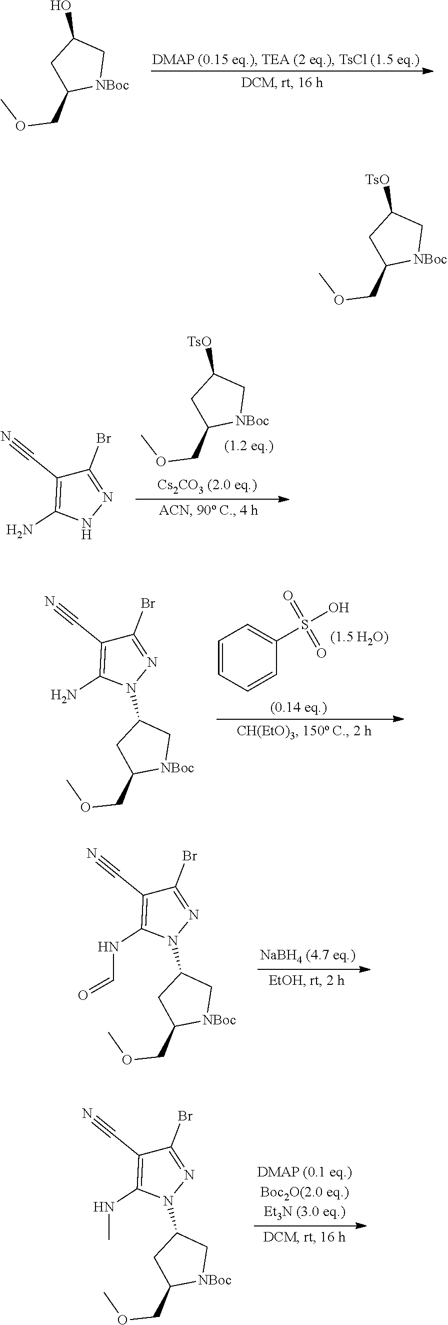

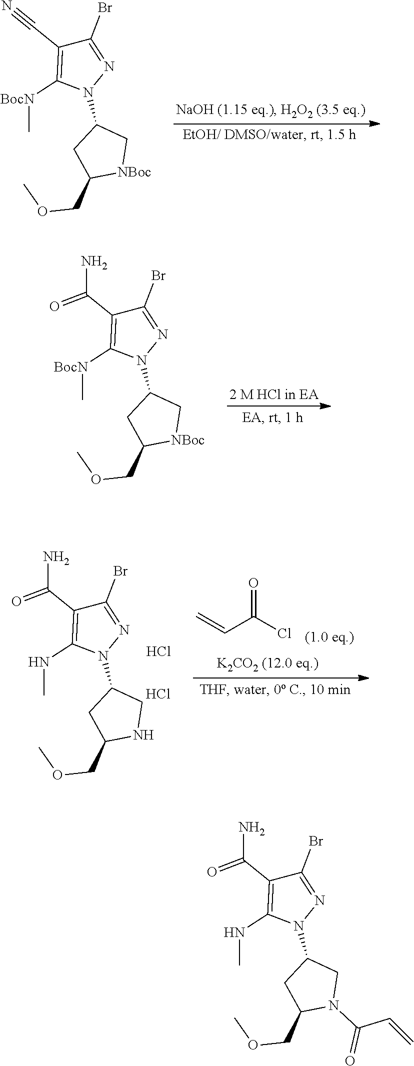

### tert-butyl (2R,4R)-2-(methoxymethyl)-4-(tosyloxy)pyrrolidine-1-carboxylate

To a stirred solution of tert-butyl (2R,4R)-4-hydroxy-2-(methoxymethyl)pyrrolidine-1-carboxylate (15.20 g, 65.72 mmol), TEA (18.27 mL, 131.44 mmol) and DMAP (1.21 g, 9.86 mmol) in DCM (150.00 mL) was added TsCl (18.79 g, 98.58 mmol) at 0° C. The reaction mixture was stirred for 16 h at room temperature under nitrogen atmosphere. The resulting mixture was diluted with water (80 mL) and extracted with DCM (3×150 mL). The combined organic layers were washed with brine (2×80 mL), dried over anhydrous Na2SO4 and filtered. The filtrate was concentrated under reduced pressure. The residue was purified by silica gel column chromatography, eluted with 33% EA in PE. The fractions that contained desired product were combined and concentrated to afford tert-butyl (2R,4R)-2-(methoxymethyl)-4-(tosyloxy)pyrrolidine-1-carboxylate (18.5 g, 73%) as a yellow oil. MS ESI calculated for C18H27NO6S [M+H]+, 386.16, found 386.10.

### tert-butyl (2R,4S)-4-(5-amino-3-bromo-4-cyano-1H-pyrazol-1-yl)-2-(methoxymethyl)pyrrolidine-1-carboxylate

To a mixture of 3-amino-5-bromo-2H-pyrazole-4-carbonitrile (7.20 g, 38.50 mmol) and Cs2CO3 (25.09 g, 77.01 mmol) in ACN (190.00 mL) was added tert-butyl (2R,4R)-2-(methoxymethyl)-4-(tosyloxy)pyrrolidine-1-carboxylate (17.81 g, 46.20 mmol). The reaction mixture was stirred for 4 h at 90° C. The reaction mixture was allowed to cool down to room temperature and filtered. The filter cake was washed with DCM (3×70 mL). The filtrate was concentrated under reduced pressure. The residue was purified by silica gel column chromatography, eluted with 46% EA in PE to get about 6.2 g product which was further purified by reverse phase-flash chromatography with the following conditions: column, C18 silica gel; mobile phase, ACN in water, 5% to 46% gradient in 30 min; detector, UV 254 nm. The fractions that contained desired product were combined and concentrated to afford tert-butyl (2R,4S)-4-(5-amino-3-bromo-4-cyano-1H-pyrazol-1-yl)-2-(methoxymethyl)pyrrolidine-1-carboxylate (5 g, 32%) as an off-white solid. MS ESI calculated for C15H22BrN5O3 [M+H]+, 400.09, 402.09; found 400.15, 402.15.

### tert-butyl (2R,4S)-4-(3-bromo-4-cyano-5-formamido-1H-pyrazol-1-yl)-2-(methoxymethyl)pyrrolidine-1-carboxylate

To a mixture of tert-butyl (2R,4S)-4-(5-amino-3-bromo-4-cyano-1H-pyrazol-1-yl)-2-(methoxymethyl)pyrrolidine-1-carboxylate (3.50 g, 8.74 mmol) in (diethoxymethoxy)ethane (70.00 mL) was added benzenesulfonic acid (0.23 g, 1.22 mmol). The reaction mixture was stirred for 2 h at 150° C. under nitrogen atmosphere. The resulting mixture was concentrated under reduced pressure. The residue was dispersed in water (50 mL). The resulting mixture was extracted with EA (3×150 mL). The combined organic layers were washed with brine (80 mL), dried over anhydrous Na2SO4 and filtered. The filtrate was concentrated to afford tert-butyl (2R,4S)-4-(3-bromo-4-cyano-5-formamido-1H-pyrazol-1-yl)-2-(methoxymethyl)pyrrolidine-1-carboxylate (3.75 g, crude) as a yellow oil which was directly used to next step without further purification. MS ESI calculated for C16H22BrN5O4 [M−H]−, 426.09, 428.09; found 426.15, 428.15.

### tert-butyl (2R,4S)-4-(3-bromo-4-cyano-5-(methylamino)-1H-pyrazol-1-yl)-2-(methoxymethyl)pyrrolidine-1-carboxylate

To a stirred solution of tert-butyl (2R,4S)-4-(3-bromo-4-cyano-5-formamido-1H-pyrazol-1-yl)-2-(methoxymethyl)pyrrolidine-1-carboxylate (3.75 g, 8.76 mmol) in EtOH (200.00 mL) was added NaBH4 (1.56 g, 41.23 mmol) at 0° C. under nitrogen atmosphere. The reaction mixture was stirred for 2 h at room temperature. The resulting mixture was quenched with brine at 0° C. The resulting mixture was extracted with EA (3×150 mL). The combined organic layers were washed with brine (70 mL), dried over anhydrous Na2SO4 and filtered. The filtrate was concentrated under reduced pressure. The residue was purified by silica gel column chromatography, eluted with 62% EA in PE. The fractions that contained desired product were combined and concentrated to afford tert-butyl (2R,4S)-4-(3-bromo-4-cyano-5-(methylamino)-1H-pyrazol-1-yl)-2-(methoxymethyl)pyrrolidine-1-carboxylate (3.3 g, 91%) as an off-white solid. MS ESI calculated for C16H24BrN5O3 [M−H]⁻, 412.11, 414.11; found 412.20, 414.20.

### tert-butyl (2R,4S)-4-(3-bromo-5-((tert-butoxycarbonyl)(methyl)amino)-4-cyano-1H-pyrazol-1-yl)-2-(methoxymethyl)pyrrolidine-1-carboxylate

To a stirred solution of tert-butyl (2R,4S)-4-[3-bromo-4-cyano-5-(methylamino)pyrazol-1-yl]-2-(methoxymethyl)pyrrolidine-1-carboxylate (1.74 g, 4.20 mmol) in DCM (40.00 mL) were added Et3N (1.75 mL, 12.61 mmol), Boc2O (2.22 g, 8.40 mmol) in DCM (2.00 mL) and DMAP (51.31 mg, 0.42 mmol) at room temperature under nitrogen atmosphere. The reaction mixture was stirred for 16 h at room temperature. The resulting mixture was concentrated under reduced pressure. The residue was purified by silica gel column chromatography, eluted with 39% EA in PE. The fractions that contained desired product were combined and concentrated to afford tert-butyl (2R,4S)-4-(3-bromo-5-((tert-butoxycarbonyl)(methyl)amino)-4-cyano-1H-pyrazol-1-yl)-2-(methoxymethyl)pyrrolidine-1-carboxylate (2.12 g, 98%) as an off-white solid. MS ESI calculated for C21H32BrN5O5 [M+H−56]+, 458.16, found 458.10.

### tert-butyl (2R,4S)-4-(3-bromo-5-((tert-butoxycarbonyl)(methyl)amino)-4-carbamoyl-1H-pyrazol-1-yl)-2-(methoxymethyl)pyrrolidine-1-carboxylate

To a stirred solution of tert-butyl (2R,4S)-4-(3-bromo-5-((tert-butoxycarbonyl)(methyl)amino)-4-cyano-1H-pyrazol-1-yl)-2-(methoxymethyl)pyrrolidine-1-carboxylate (3.17 g, 6.16 mmol) in EtOH (23.50 mL) and DMSO (4.70 mL) were added 0.5 M NaOH (8.14 mL, 7.08 mmol) and H2O2 (30%) (1.69 mL, 14.77 mmol) at 0° C. The reaction mixture was stirred for 30 min at 0° C. and stirred for 1.5 h at room temperature. The resulting mixture was extracted with EA (3×90 mL). The combined organic layers were washed with brine (2×60 mL), dried over anhydrous Na2SO4 and filtered. The filtrate was concentrated under reduced pressure. The residue was purified by silica gel column chromatography, eluted with 60% EA in PE. The fractions that contained desired product were combined and concentrated to afford tert-butyl (2R,4S)-4-(3-bromo-5-((tert-butoxycarbonyl)(methyl)amino)-4-carbamoyl-1H-pyrazol-1-yl)-2-(methoxymethyl)pyrrolidine-1-carboxylate (3.09 g, 94%) as an off-white solid. MS ESI calculated for C21H34BrN5O6 [M+H−156]+, 376.17, found 376.00.

### 3-bromo-1-((3S,5R)-5-(methoxymethyl)pyrrolidin-3-yl)-5-(methylamino)-1H-pyrazole-4-carboxamide dihydrochloride

To a stirred solution of tert-butyl (2R,4S)-4-(3-bromo-5-((tert-butoxycarbonyl)(methyl)amino)-4-carbamoyl-1H-pyrazol-1-yl)-2-(methoxymethyl)pyrrolidine-1-carboxylate (1.68 g, 3.16 mmol) in EA (8.5 mL) was added 2 M hydrogen chloride solution in EA (17 mL) dropwise at 0° C. under nitrogen atmosphere. The reaction mixture was stirred for 1 h at room temperature. The precipitated solids were collected by filtration and washed with EA (3×30 mL). The filter cake was dried to afford 3-bromo-1-((3S,5R)-5-(methoxymethyl)pyrrolidin-3-yl)-5-(methylamino)-1H-pyrazole-4-carboxamide dihydrochloride (1.3 g, crude) as an off-white solid. MS ESI calculated for C11H18BrN5O2 [M+H]+, 332.06, found 332.15.

### 1-((3S,5R)-1-acryloyl-5-(methoxymethyl)pyrrolidin-3-yl)-3-bromo-5-(methylamino)-1H-pyrazole-4-carboxamide

To a stirred mixture of 3-bromo-1-((3S,5R)-5-(methoxymethyl)pyrrolidin-3-yl)-5-(methylamino)-1H-pyrazole-4-carboxamide dihydrochloride (1.20 g, 2.96 mmol) in THF (24.00 mL) were added 2.5 M aqueous K2CO3 (14.40 mL, 36 mmol) and acryloyl chloride (0.23 g, 2.96 mmol) in THF (6.00 mL) at 0° C. The reaction mixture was stirred for 10 min at 0° C. The resulting mixture was extracted with EA (3×100 mL). The combined organic layers were washed with brine (2×60 mL), dried over anhydrous Na2SO4 and filtered. The filtrate was concentrated under reduced pressure. The residue was purified by silica gel column chromatography, eluted with CH2Cl2/MeOH (10:1). The fractions that contained desired product were combined and concentrated to afford 1-((3S,5R)-1-acryloyl-5-(methoxymethyl)pyrrolidin-3-yl)-3-bromo-5-(methylamino)-1H-pyrazole-4-carboxamide (0.89 g, 78%) as an off-white solid. MS ESI calculated for C14H20BrN5O3 [M+H]+, 386.07, found 386.05.

### Example 1: 1-[(3S,5R)-5-(methoxymethyl)-1-(prop-2-enoyl)pyrrolidin-3-yl]-3-[2-(2-methyl-3H-1,3-benzodiazol-5-yl)ethynyl]-5-(methylamino)pyrazole-4-carboxamide

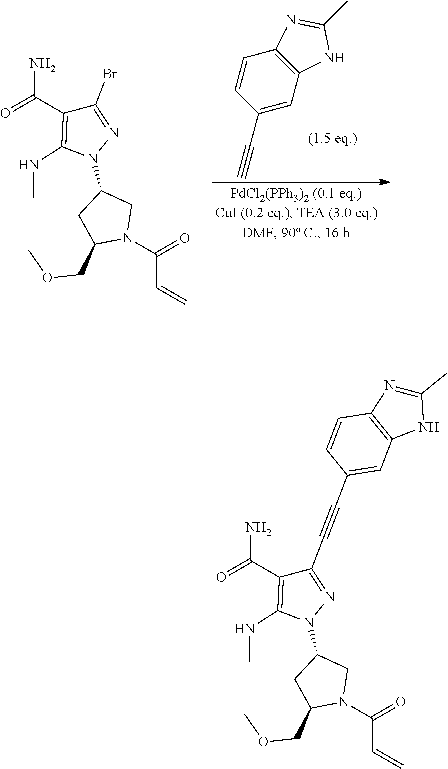 [SMILES: C=CC(=O)N1C[C@@H](n2nc(Br)c(C(N)=O)c2NC)C[C@@H]1COC ; C#Cc1ccc2nc(C)[nH]c2c1 ; C=CC(=O)N1C[C@@H](n2nc(C#Cc3ccc4nc(C)[nH]c4c3)c(C(N)=O)c2NC)C[C@@H]1COC]

To a mixture of 3-bromo-1-[(3S,5R)-5-(methoxymethyl)-1-(prop-2-enoyl)pyrrolidin-3-yl]-5-(methylamino)pyrazole-4-carboxamide (0.10 g, 0.26 mmol), 5-ethynyl-2-methyl-3H-1,3-benzodiazole (60.66 mg, 0.39 mmol), CuI (9.86 mg, 0.05 mmol) in DMF (2.50 mL) were added Pd(PPh3)2Cl2 (18.17 mg, 0.03 mmol) and TEA (0.11 mL, 1.07 mmol). The reaction mixture was degassed with nitrogen for three times and stirred for 16 h at 90° C. The resulting mixture was diluted with water (20 mL) and extracted with EtOAc (3×20 mL). The combined organic layers were washed with brine (50 mL), dried over anhydrous Na2SO4 and filtered. The filtrate was concentrated under reduced pressure. The residue was purified by Prep-HPLC with the following conditions Column: SunFire Prep C18 OBD Column, 19×150 mm 5 um 10 nm; Mobile Phase A: Water (0.1% FA), Mobile Phase B: ACN; Flow rate: 20 ml/min; Gradient: 20 B to 50 B in 4.3 min. The fractions that contained desired product were combined and concentrated to afford 1-[(3S,5R)-5-(methoxymethyl)-1-(prop-2-enoyl)pyrrolidin-3-yl]-3-[2-(2-methyl-3H-1,3-benzodiazol-5-yl)ethynyl]-5-(methylamino)pyrazole-4-carboxamide (35.8 mg, 29%) as an off-white solid. MS ESI calculated for C24H27N7O3 [M+H]+, 462.22, found 462.15. H-NMR (400 MHz, DMSO-d6+D2O): δ 7.70 (s, 1H), 7.52 (d, J=8.0 Hz, 1H), 7.34-7.32 (m, 1H), 6.73-6.55 (m, 1H), 6.19-6.14 (m, 1H), 5.71-5.67 (m, 1H), 5.23-5.10 (m, 1H), 4.59-4.35 (m, 1H), 3.90-3.85 (m, 1H), 3.76-3.61 (m, 1H), 3.59-3.31 (m, 2H), 3.30 (d, J=5.2 Hz, 3H), 2.93 (d, J=4.4 Hz, 3H), 2.56 (s, 3H), 2.51-2.33 (m, 1H), 2.31-2.27 (m, 1H).

### Example 2: (S)-1-(1-acryloylpyrrolidin-3-yl)-3-((2-methyl-1H-benzo[d]imidazol-6-yl)ethynyl)-5-(methylamino)-1H-pyrazole-4-carboxamide

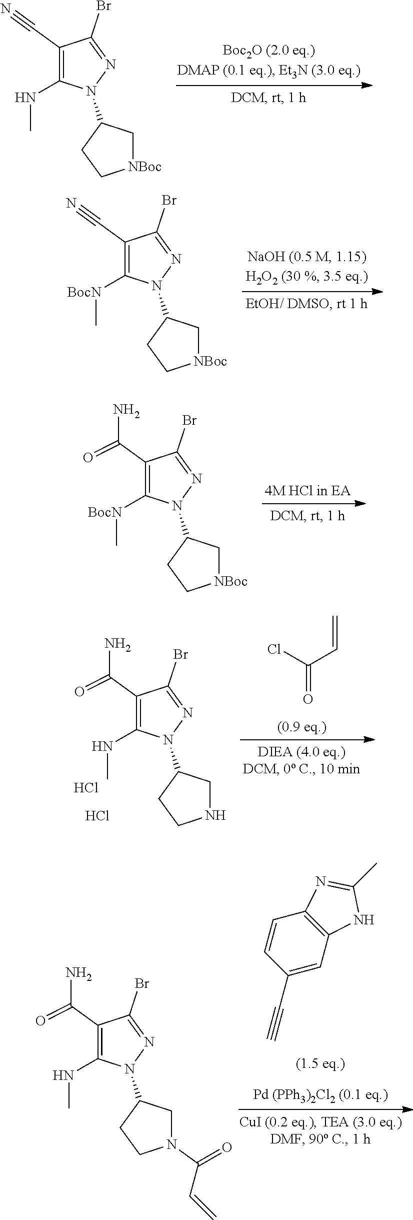

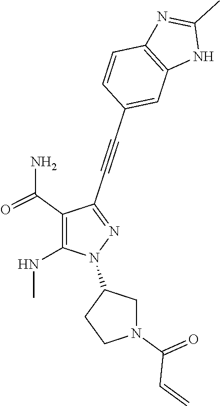

### Step 1: tert-butyl (S)-3-(3-bromo-5-((tert-butoxycarbonyl)(methyl)amino)-4-cyano-1H-pyrazol-1-yl)pyrrolidine-1-carboxylate

To a stirred solution of tert-butyl (3S)-3-[3-bromo-4-cyano-5-(methylamino)pyrazol-1-yl]pyrrolidine-1-carboxylate (0.5 g, 1.35 mmol) in DCM (10.00 mL) were added TEA (0.56 mL, 4.03 mmol), Boc2O (0.56 g, 2.70 mmol) in DCM (3.00 mL) and DMAP (16.50 mg, 0.14 mmol) at room temperature under nitrogen atmosphere. The reaction mixture was stirred for 1 h. The resulting mixture was concentrated under reduced pressure. The residue was purified by silica gel column chromatography, eluted with 31% EA in PE, The fractions that contained desired product were concentrated to afford tert-butyl (S)-3-(3-bromo-5-((tert-butoxycarbonyl)(methyl)amino)-4-cyano-1H-pyrazol-1-yl)pyrrolidine-1-carboxylate (0.6 g, 94%) as an off-white solid. MS ESI calculated for C19H28BrN5O4 [M+H−112]+, 470.13, found 358.10.

### Step 2: tert-butyl (S)-3-(3-bromo-5-((tert-butoxycarbonyl)(methyl)amino)-4-carbamoyl-1H-pyrazol-1-yl)pyrrolidine-1-carboxylate

To a stirred solution of tert-butyl (S)-3-(3-bromo-5-((tert-butoxycarbonyl)(methyl)amino)-4-cyano-1H-pyrazol-1-yl)pyrrolidine-1-carboxylate (0.5 g, 1.06 mmol) in EtOH (4.00 mL) and DMSO (0.80 mL) were added 0.5 M NaOH (2.44 mL, 1.22 mmol) and H2O2 (30%) (0.29 mL, 3.73 mmol) at 0° C. The reaction mixture was stirred for 1 h at ambient temperature. The resulting mixture was diluted with water (5 mL), extracted with EtOAc (3×20 mL). The combined organic layers were washed with brine (3×15 mL), dried over anhydrous Na2SO4 and filtered. The filtrate was concentrated under reduced pressure. The residue was purified by silica gel column chromatography, eluted with 60% EA in PE, The fractions that contained desired product were combined and concentrated to afford tert-butyl (S)-3-(3-bromo-5-((tert-butoxycarbonyl)(methyl)amino)-4-carbamoyl-1H-pyrazol-1-yl)pyrrolidine-1-carboxylate (0.56 g, 97%) as an off-white solid. MS ESI calculated for C19H30BrN5O5 [M+H−156]+, 488.13, found 332.05.

### Step 3: (S)-3-bromo-5-(methylamino)-1-(pyrrolidin-3-yl)-1H-pyrazole-4-carboxamide dihydrochloride

To a stirred solution of tert-butyl (S)-3-(3-bromo-5-((tert-butoxycarbonyl)(methyl)amino)-4-carbamoyl-1H-pyrazol-1-yl)pyrrolidine-1-carboxylate (0.25 g, 0.51 mmol) in DCM (1.50 mL) was added hydrogen chloride solution 4 M in EA (3 mL) dropwise at 0° C. under nitrogen atmosphere. The reaction mixture was stirred for 1 h at room temperature under nitrogen atmosphere. The precipitated solids were collected by filtration and washed with DCM (3×10 mL). The solid was dried under reduced pressure to afford (S)-3-bromo-5-(methylamino)-1-(pyrrolidin-3-yl)-1H-pyrazole-4-carboxamide dihydrochloride (0.20 g, crude) as an off-white solid. MS ESI calculated for C9H14BrN5O [M+H]+, 288.04, found 288.10.

### Step 4: (S)-1-(1-acryloylpyrrolidin-3-yl)-3-bromo-5-(methylamino)-1H-pyrazole-4-carboxamide

To a stirred solution of (S)-3-bromo-5-(methylamino)-1-(pyrrolidin-3-yl)-1H-pyrazole-4-carboxamide dihydrochloride (0.16 g, 0.44 mmol) in DCM (5.00 mL) were added acryloyl chloride (36.10 mg, 0.40 mmol) and DIEA (0.32 mL, 1.84 mmol) dropwise at 0° C. The reaction mixture was stirred for 10 min at 0° C. The resulting mixture was concentrated under reduced pressure. The residue was diluted with water (5 mL), extracted with EtOAc (3×10 mL). The combined organic layers were washed with brine (2×10 mL), dried over anhydrous Na2SO4 and filtered. The filtrate was concentrated under reduced pressure. The residue was purified by silica gel column chromatography, eluted with CH2Cl2/MeOH (10:1). The fractions that contained desired product were combined and concentrated to afford (S)-1-(1-acryloylpyrrolidin-3-yl)-3-bromo-5-(methylamino)-1H-pyrazole-4-carboxamide (0.12 g, 75%) as an off-white solid. MS ESI calculated for C12H18BrN5O2 [M+H]+, 342.05, found 342.00.

### Step 5: (S)-1-(1-acryloylpyrrolidin-3-yl)-3-((2-methyl-1H-benzo[d]imidazol-6-yl)ethynyl)-5-(methylamino)-1H-pyrazole-4-carboxamide

To a stirred mixture of (S)-1-(1-acryloylpyrrolidin-3-yl)-3-bromo-5-(methylamino)-1H-pyrazole-4-carboxamide (90.00 mg, 0.26 mmol), 5-ethynyl-2-methyl-3H-1,3-benzodiazole (61.62 mg, 0.40 mmol), CuI (10.02 mg, 0.05 mmol) and Pd(PPh3)2Cl2 (18.46 mg, 0.03 mmol) in DMF (2.00 mL) was added TEA (0.09 mL, 0.89 mmol). The reaction mixture was degassed with argon for three times and stirred for 1 h at 90° C. The resulting mixture was concentrated under reduced pressure. The residue was purified by Prep-HPLC with the following conditions Column: XBridge Shield RP18 OBD Column, 30*150 mm, 5 um; Mobile Phase A: Water (10 mmol/L NH4HCO3), Mobile Phase B: ACN; Flow rate: 20 mL/min; Gradient: 35 B to 70 B in 10 min; 210/254 nm; RT1: 9.75. The fractions that contained desired product were combined and concentrated to afford (S)-1-(1-acryloylpyrrolidin-3-yl)-3-((2-methyl-1H-benzo[d]imidazol-6-yl)ethynyl)-5-(methylamino)-1H-pyrazole-4-carboxamide (22 mg, 20%) as an off-white solid. MS ESI calculated for C22H23N7O2 [M+H]+, 418.19, found 418.10. H-NMR (300 MHz, CDCl3) δ 7.70 (s, 1H), 7.51-7.48 (m, 1H), 7.38-7.26 (d, J=8.3 Hz, 1H), 6.97 (s, 1H), 6.66-6.31 (m, 3H), 5.77-5.66 (m, 1H), 5.48 (s, 1H), 5.04-4.99 (m, 1H), 4.16-3.94 (m, 3H), 3.82-3.62 (m, 1H), 2.99 (t, J=6.1 Hz, 3H), 2.65 (s, 4H), 2.40 (m, 2H).

### Example 3: 3-[2-(1-ethyl-2-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

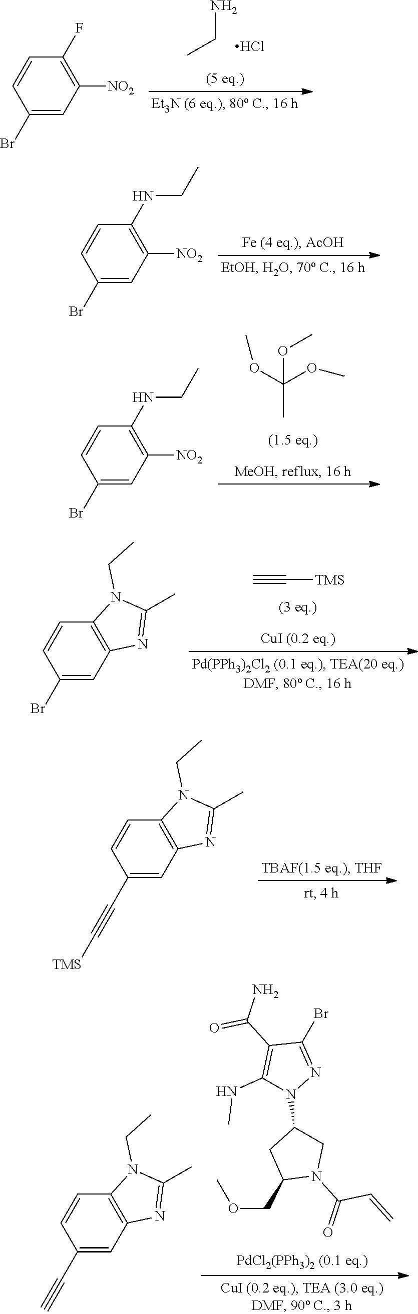

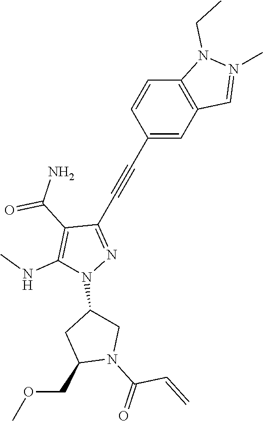

### Step 1: 4-bromo-N-ethyl-2-nitroaniline

A solution of 4-bromo-1-fluoro-2-nitrobenzene (5.00 g, 22.73 mmol) and ethylamine hydrochloride (9.27 g, 113.64 mmol) in Et3N (13.80 g, 136.38 mmol) was stirred for 16 h at 80° C. The resulting mixture was concentrated under reduced pressure. The residue was quenched with sat. NH4HCO3 (aq.) at room temperature. The resulting mixture was extracted with EtOAc (3×50 mL). The combined organic layers were dried over anhydrous Na2SO4 and filtered. The filtrate was concentrated under reduced pressure to afford 4-bromo-N-ethyl-2-nitroaniline (5 g, 89%) as a red solid which was used in the next step without further purification. MS ESI calculated for C8H9BrN202 [M+H]+, 244.98, 246.98, found 245.05, 247.05.

### Step 2: 4-bromo-N1-ethylbenzene-1,2-diamine

To a mixture of Fe (2.73 g, 48.96 mmol) in EtOH (66.00 mL) was added AcOH (3.30 mL, 54.90 mmol). The resulting mixture was stirred for 30 min at 70° C. under nitrogen atmosphere. To the above mixture was added 4-bromo-N-ethyl-2-nitroaniline (3.00 g, 12.24 mmol). The reaction mixture was stirred for 16 h at 70° C. The resulting mixture was filtered, the filter cake was washed with THF. The filtrate was concentrated under reduced pressure. The residue was quenched by the addition of sat. NH4HCO3 (aq.) (50 mL) at room temperature. The resulting mixture was extracted with EtOAc (3×50 mL), dried over anhydrous Na2SO4 and filtered. The filtrate was concentrated under reduced pressure to afford 4-bromo-N1-ethylbenzene-1,2-diamine (2 g, 75%) as a brown solid which was used in next step without further purification. MS ESI calculated for C8H11BrN2 [M+H]+, 215.01, 217.01, found 215.10, 217.10.

### Step 3: 5-bromo-1-ethyl-2-methyl-1,3-benzodiazole

To a stirred solution of 4-bromo-N1-ethylbenzene-1,2-diamine (1.30 g, 6.04 mmol) in MeOH (13.00 mL) was added 1,1,1-trimethoxyethane (1.09 g, 0.01 mmol). The reaction mixture was stirred for 16 h at 70° C. The resulting mixture was concentrated under reduced pressure. The residue was purified by silica gel column chromatography, eluted with CH2Cl2/MeOH (12:1). The fractions that contained desired product were combined and concentrated to afford 5-bromo-1-ethyl-2-methyl-1,3-benzodiazole (1.00 g, 69%) as a light-yellow solid. MS ESI calculated for C10H11BrN2 [M+H]+, 239.01, 241.01, found 238.90, 240.90.

### Step 4: 1-ethyl-2-methyl-5-[2-(trimethylsilyl) ethynyl]-1,3-benzodiazole

To a stirred mixture of 5-bromo-1-ethyl-2-methyl-1,3-benzodiazole (1.50 g 6.27 mmol), trimethylsilylacetylene (1.85 g, 0.02 mmol), CuI (0.24 g, 1.26 mmol) and Pd(PPh3)2Cl2 (0.88 g, 1.25 mmol) in DMF (15.00 mL) was added TEA (12.70 g 0.13 mmol). The reaction mixture was degassed with argon for three times and stirred for 16 h at 80° C. The resulting mixture was extracted with EtOAc (3×30 mL). The combined organic layers were washed with brine (30 mL), dried over anhydrous Na2SO4 and filtered. The filtrate was concentrated under reduced pressure. The residue was purified by silica gel column chromatography, eluted with CH2Cl2/MeOH (12:1). The fractions that contained desired product were combined and concentrated to afford 1-ethyl-2-methyl-5-[2-(trimethylsilyl) ethynyl]-1,3-benzodiazole (0.5 g, 31%) as a light-yellow solid. MS ESI calculated for C15H20N2Si [M+H]+, 257.14, found 257.15.

### Step 5: 1-ethyl-5-ethynyl-2-methyl-1,3-benzodiazole

To a solution of 1-ethyl-2-methyl-5-[2-(trimethylsilyl)ethynyl]-1,3-benzodiazole (0.5 g, 1.95 mmol) in THF (5.00 mL) was added TBAF (0.76 g, 2.92 mmol) at 0° C. under nitrogen atmosphere. The reaction mixture was stirred for 4 h at room temperature. The resulting mixture was concentrated under reduced pressure. The residue was purified by silica gel column chromatography, eluted with CH2Cl2/MeOH (12:1). The fractions that contained desired product were combined and concentrated to afford 1-ethyl-5-ethynyl-2-methyl-1,3-benzodiazole (0.30 g, 83%) as a light-yellow solid. MS ESI calculated for C12H12N2 [M+H]+, 185.10, found 185.10.

### Step 6: 3-[2-(1-ethyl-2-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a stirred mixture of 3-bromo-1-[(3S,5R)-5-(methoxymethyl)-1-(prop-2-enoyl)pyrrolidin-3-yl]-5-(methylamino)pyrazole-4-carboxamide (0.20 g, 0.52 mmol), 1-ethyl-5-ethynyl-2-methyl-1,3-benzodiazole (0.14 g 0.78 mmol), CuI (19.72 mg, 0.10 mmol) and Pd(PPh3)2Cl2 (36.34 mg, 0.05 mmol) in DMF (3 mL) was added TEA (0.16 g, 1.55 mmol). The reaction mixture was degassed with argon for three times and stirred for 3 h at 90° C. The resulting mixture was concentrated under reduced pressure. The residue was purified by Prep-HPLC with the following conditions Column: XBridge Prep C18 OBD Column, 19×150 mm 5 um; Mobile Phase A: Water (10 mmol/L NH4HCO3), Mobile Phase B: ACN; Flow rate: 20 mL/min; Gradient: 50 B to 80 B in 4.3 min; 210/254 nm; RT1:4.02. The fractions that contained desired product were concentrated to afford 3-[2-(1-ethyl-2-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (50 mg, 19%) as a white solid. MS ESI calculated for C26H31N7O3 [M+H]+, 490.25, found 490.25. H-NMR (300 MHz, CDCl3) δ 7.90 (s, 1H), 7.43 (d, J=8.2 Hz, 1H), 6.98 (s, 1H), 6.98-6.77 (m, 2H), 6.54-6.37 (m, 2H), 5.70 (dd, J=8.2, 4.2 Hz, 1H), 5.58-5.26 (m, 2H), 4.56 (d, J=9.0 Hz, 1H), 4.26-3.85 (m, 5H), 3.54-3.34 (m, 4H), 3.05-3.01 (m, 3H), 2.79-2.53 (m, 4H), 2.33-2.29 (m, 1H), 1.43 (t, J=7.2 Hz, 3H).

### Example 4: 1-[(3S,5R)-5-(methoxymethyl)-1-(prop-2-enoyl)pyrrolidin-3-yl]-3-[2-(1-methyl-1,3-benzodiazol-5-yl)ethynyl]-5-(methylamino)pyrazole-4-carboxamide

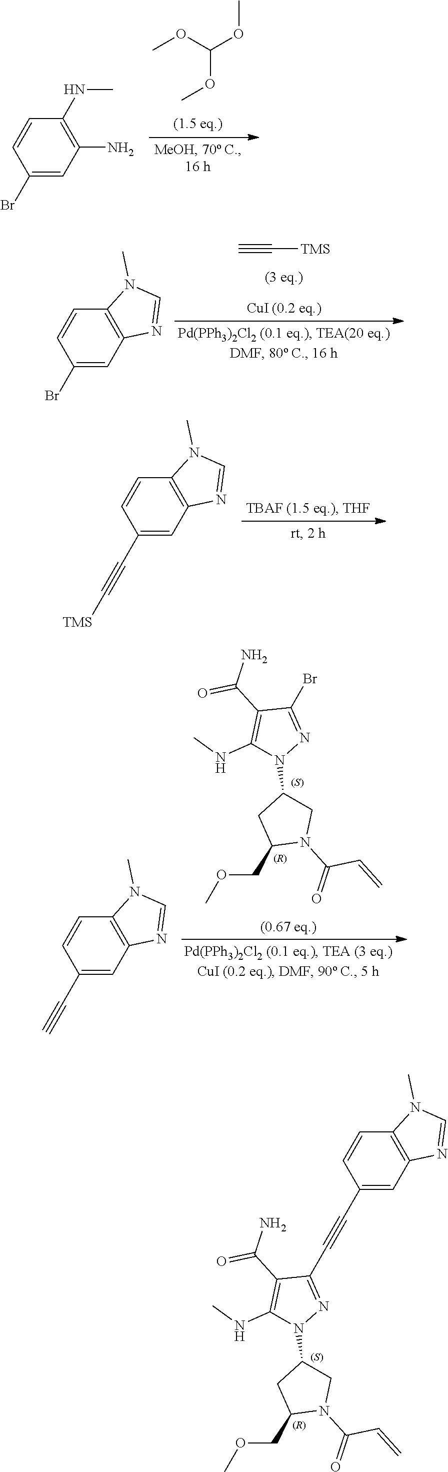

### Step 1: 5-bromo-1-methyl-1,3-benzodiazole

To a stirred solution of 4-bromo-N1-methylbenzene-1,2-diamine (1.10 g, 5.47 mmol) in MeOH (11.00 mL) was added trimethyl orthoformate (0.87 g, 8.21 mmol). The reaction mixture was stirred for 16 h at 70° C. under nitrogen atmosphere. The resulting mixture was concentrated under reduced pressure. The residue was purified by silica gel column chromatography, eluted with 4% MeOH in DCM. The fractions that contained desired product were combined and concentrated to afford 5-bromo-1-methyl-1,3-benzodiazole (1.02 g, 79%) as a brown solid. MS ESI calculated for C8H7BrN2 [M+H]+, 210.98, found 210.95.

### Step 2: 1-methyl-5-[2-(trimethylsilyl)ethynyl]-1,3-benzodiazole

To a mixture of 5-bromo-1-methyl-1,3-benzodiazole (1.00 g, 4.74 mmol), trimethylsilylacetylene (2.01 mL, 20.45 mmol), CuI (0.18 g, 0.95 mmol and Pd(PPh3)2Cl2 (0.33 g, 0.47 mmol) in DMF (20.00 mL) was added TEA (13.17 mL, 130.16 mmol). The reaction mixture was degassed with argon for three times and stirred for 16 h at 80° C. The resulting mixture was diluted with water (60 mL) and extracted with EA (3×70 mL). The combined organic layers were washed with brine (2×100 mL), dried over anhydrous Na2SO4, filtered and concentrated under reduced pressure. The residue was purified by silica gel column chromatography, eluted with 8% MeOH in DCM. The crude product was purified by reverse flash chromatography with the following conditions: column, C18 silica gel; mobile phase, NH4HCO3 in water, 30% to 70% gradient in 30 min; detector, UV 254 nm. The fractions that contained desired product were combined and concentrated to afford 1-methyl-5-[2-(trimethylsilyl)ethynyl]-1,3-benzodiazole (0.51 g, 42%) as a light brown solid. MS ESI calculated for C13H16N2Si [M+H]+, 229.11, found 229.20.

### Step 3: 5-ethynyl-1-methylindazole

To a stirred solution of 1-methyl-5-[2-(trimethylsilyl)ethynyl]indazole (0.51 g, 2.25 mmol) in THF (5.00 mL) was added TBAF (1 M in THF) (3.38 mL, 3.37 mmol) dropwise at 0° C. under nitrogen atmosphere. The resulting mixture was stirred for 2 h at room temperature. The resulting mixture was diluted with water (20 mL), extracted with EtOAc (3×20 mL). The combined organic layers were washed with brine (50 mL), dried over anhydrous Na2SO4 and filtered. The filtrate was concentrated under reduced pressure. The residue was purified by silica gel column chromatography, eluted with CH2Cl2/MeOH (20:1). The fractions that contained desired product were combined and concentrated to afford 5-ethynyl-1-methylindazole (0.32 g, 81%) as an off-white solid. MS ESI calculated for C10H8N2 [M+H]+, 157.07, found 157.10.

### Step 4: 1-[(3S,5R)-5-(methoxymethyl)-1-(prop-2-enoyl)pyrrolidin-3-yl]-3-[2-(1-methyl-1,3-benzodiazol-5-yl)ethynyl]-5-(methylamino)pyrazole-4-carboxamide

To a stirred mixture of 3-bromo-1-[(3S,5R)-5-(methoxymethyl)-1-(prop-2-enoyl)pyrrolidin-3-yl]-5-(methylamino)pyrazole-4-carboxamide (0.23 g, 0.59 mmol), 5-ethynyl-1-methyl-1,3-benzodiazole (0.14 g, 0.89 mmol), CuI (22.68 mg, 0.12 mmol) and Pd(PPh3)2Cl2 (41.80 mg, 0.06 mmol) in DMF (4.00 mL) was added TEA (0.25 mL, 2.45 mmol) dropwise at room temperature. The reaction mixture was degassed with argon for three times and stirred for 5 h at 90° C. The resulting mixture was concentrated under reduced pressure. The residue was purified by reverse flash chromatography with the following conditions: column, C18 silica gel; mobile phase, NH4HCO3 in water, 20% to 55% gradient in 30 min; detector, UV 254 nm. The crude product was purified by Prep-HPLC with the following conditions Column: SunFire Prep C18 OBD Column, 19×150 mm 5 μm 10 nm; Mobile Phase A: Water (0.1% FA), Mobile Phase B: ACN; Flow rate: 20 mL/min; Gradient: 35 B to 70 B in 4.3 min; 210/254 nm; RT1: 4.12 min. The fractions that contained desired product were combined and concentrated to afford 1-[(3S,5R)-5-(methoxymethyl)-1-(prop-2-enoyl)pyrrolidin-3-yl]-3-[2-(1-methyl-1,3-benzodiazol-5-yl)ethynyl]-5-(methylamino)pyrazole-4-carboxamide (0.11 g, 37%) as an off-white solid. MS ESI calculated for C24H27N7O3 [M+H]+, 462.22, found 462.10. H-NMR (400 MHz, DMSO-d6): δ 12.75 (brs, 1H), 8.33-8.30 (m, 1H), 8.15-8.13 (m, 1H), 7.92-7.86 (m, 1H), 7.66 (d, J=8.4 Hz, 1H), 7.47 (d, J=8.4 Hz, 1H), 7.38-7.34 (m, 1H), 6.83-6.76 (m, 1H), 6.73-6.54 (m, 1H), 6.19-6.14 (m, 1H), 5.71-5.67 (m, 1H), 5.27-5.25 (m, 1H), 4.59-4.35 (m, 1H), 4.03-4.02 (m, 1H), 3.93-3.91 (m, 4H), 3.86-3.71 (m, 1H), 3.45-3.30 (m, 3H), 2.95 (t, J=5.1 Hz, 3H), 2.61-2.51 (m, 1H), 2.33-2.24 (m, 1H).

### Example 5: 1-((3S,5R)-1-acryloyl-5-(methoxymethyl)pyrrolidin-3-yl)-3-((1,2-dimethyl-1H-benzo[d]imidazol-5-yl)ethynyl)-5-(methylamino)-1H-pyrazole-4-carboxamide

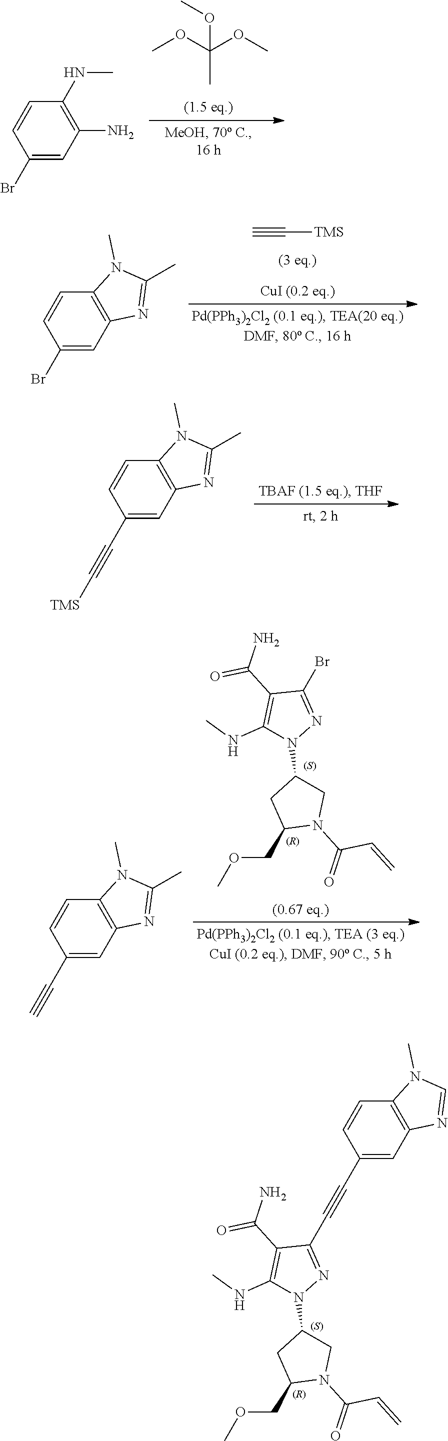

### Step 1: 5-bromo-1,2-dimethyl-1,3-benzodiazole

To a stirred solution of 4-bromo-N1-methylbenzene-1,2-diamine (4.00 g, 19.89 mmol) in MeOH (40.00 mL) was added 1,1,1-trimethoxyethane (3.59 g, 29.84 mmol). The reaction mixture was stirred for 16 h at 70° C. under nitrogen atmosphere. The resulting mixture was concentrated under reduced pressure. The residue was purified by silica gel column chromatography, eluted with 6% MeOH in DCM. The fractions contained desired product were combined and concentrated to afford 5-bromo-1,2-dimethyl-1,3-benzodiazole (3.91 g, 78%) as a light yellow solid. MS ESI calculated for C9H9BrN2 [M+H]+, 224.99, 226.99; found 225.00, 227.00.

### Step 2: 1,2-dimethyl-5-[2-(trimethylsilyl)ethynyl]-1,3-benzodiazole

To a stirred mixture of 5-bromo-1,2-dimethyl-1,3-benzodiazole (2.53 g, 11.24 mmol), trimethylsilylacetylene (4.77 mL, 48.52 mmol), CuI (428.13 mg, 2.25 mmol) and Pd(PPh₃)₂Cl₂ (788.94 mg, 1.12 mmol) in DMF (50.00 mL) was added TEA (31.25 mL, 308.79 mmol). The reaction mixture was degassed with nitrogen for three times and stirred for 16 h at 80° C. The resulting mixture was diluted with water (200 mL) and extracted with EA (3×200 mL). The combined organic layers were washed with brine (2×200 mL), dried over anhydrous Na₂SO₄, filtered and concentrated under reduced pressure. The residue was purified by silica gel column chromatography, eluted with 8% MeOH in DCM. The fractions contained desired product were combined and concentrated. The residue was purified by reverse flash chromatography with the following conditions: column, C18 silica gel; mobile phase, NH₄HCO₃ in water, 30% to 70% gradient in 35 min; detector, UV 254 nm. The fractions contained desired product were combined and concentrated to afford 1,2-dimethyl-5-[2-(trimethylsilyl)ethynyl]-1,3-benzodiazole (0.52 g, 17%) as a brown solid. MS ESI calculated for C₁₄H₁₈N₂Si [M+H]⁺, 243.12, found 243.00.

### Step 3: 5-ethynyl-1,2-dimethyl-1,3-benzodiazole

To a stirred solution of 1,2-dimethyl-5-[2-(trimethylsilyl)ethynyl]-1,3-benzodiazole (1.06 g 4.37 mmol) in THF (20.00 mL) was added TBAF (6.56 mL, 6.56 mmol) dropwise at 0° C. under nitrogen atmosphere. The reaction mixture was stirred for 2 h at room temperature. The resulting mixture was concentrated under reduced pressure. The residue was diluted with water (100 mL), extracted with EtOAc (3×100 mL). The combined organic layers were washed with brine (200 mL), dried over anhydrous Na₂SO₄ and filtered. The filtrate was concentrated under reduced pressure. The residue was purified by silica gel column chromatography, eluted with CH₂Cl₂/MeOH (20:1). The fractions contained desired product were combined and concentrated to afford 5-ethynyl-1,2-dimethyl-1,3-benzodiazole (0.68 g, 81%) as an off-white solid. MS ESI calculated for C₁₁H₁₀N₂ [M+H]⁺, 171.08, found 171.15.

### Step 4: 3-[2-(1,2-dimethyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a stirred mixture of 3-bromo-1-[(3S,5R)-5-(methoxymethyl)-1-(prop-2-enoyl)pyrrolidin-3-yl]-5-(methylamino)pyrazole-4-carboxamide (0.27 g, 0.70 mmol), 5-ethynyl-1,2-dimethyl-1,3-benzodiazole (0.18 g, 1.05 mmol), CuI (26.63 mg, 0.14 mmol) and Pd(PPh₃)₂Cl₂ (49.06 mg, 0.07 mmol) in DMF (4.50 mL) was added TEA (0.29 mL, 2.88 mmol). The reaction mixture was degassed with argon for three times and stirred for 5 h at 90° C. The resulting mixture was diluted with water (30 mL) and extracted with EA (3×50 mL). The combined organic layers were washed with brine (2×100 mL), dried over anhydrous Na₂SO₄, filtered and concentrated under reduced pressure. The residue was purified by reverse flash chromatography with the following conditions: column, C18 silica gel; mobile phase, NH₄HCO₃ in water, 25% to 40% gradient in 25 min; detector, UV 254 nm. The fractions contained desired product were combined and concentrated. The residue was purified by Prep-HPLC with the following conditions Column: XBridge Prep C18 OBD Column, 19×150 mm 5 um; Mobile Phase A: Water (10 mmol/L NH₄HCO₃), Mobile Phase B: ACN; Flow rate: 20 mL/min; Gradient: 35 B to 70 B in 4.3 min; 210/254 nm; RT1: 4.02. The fractions contained desired product were combined and concentrated to afford 3-[2-(1,2-dimethyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (0.16 g, 46%) as an off-white solid. MS ESI calculated for C₂₅H₂₉N₇O₃ [M+H]⁺, 476.23, found 476.25. H-NMR (400 MHz, DMSO-d₆): δ 7.76 (s, 1H), 7.58 (d, J=8.3 Hz, 1H), 7.43-7.38 (m, 2H), 6.76-6.59 (m, 3H), 6.19 (d, J=16.5 Hz, 1H), 5.71 (d, J=10.2 Hz, 1H), 5.27-5.21 (m, 1H), 4.48-4.35 (m, 1H), 4.11-3.82 (m, 1H), 3.77-3.71 (m, 4H), 3.67-3.38 (m, 2H), 3.34 (d, J=1.1 Hz, 3H), 2.96 (t, J=4.5 Hz, 3H), 2.66-2.50 (m, 4H), 2.29-2.21 (m, 1H).

### Example 6: 1-((3S,5R)-1-acryloyl-5-(methoxymethyl)pyrrolidin-3-yl)-3-((1-ethyl-1H-benzo[d]imidazol-5-yl)ethynyl)-5-(methylamino)-1H-pyrazole-4-carboxamide

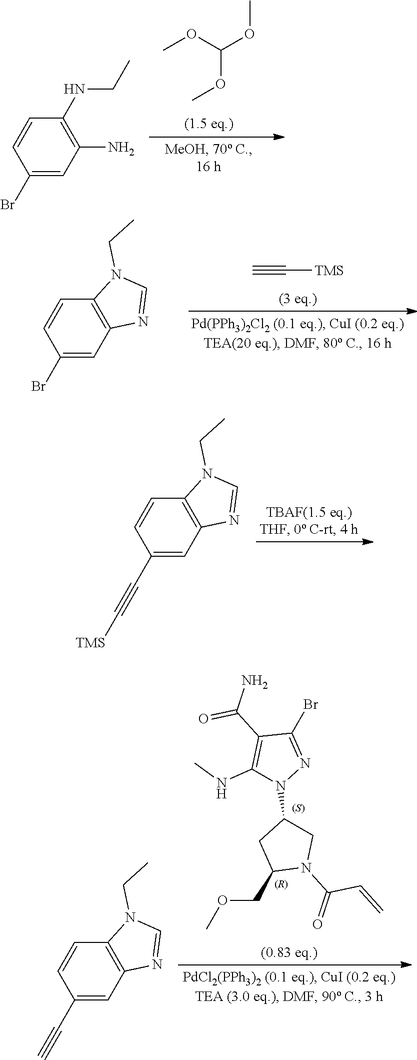

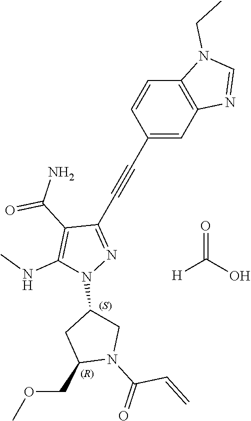

### Step 1: 5-bromo-1-methyl-1,3-benzodiazole

To a stirred solution of 4-bromo-N1-ethylbenzene-1,2-diamine (1.08 g, 5.02 mmol) in MeOH (10.00 mL) was added trimethyl orthoformate (0.80 g, 7.53 mmol). The reaction mixture was stirred for 16 h at 70° C. The resulting mixture was concentrated under reduced pressure. The residue was purified by silica gel column chromatography, eluted with CH₂Cl₂/MeOH (12:1). The fractions contained desired product were combined and concentrated to afford 5-bromo-1-ethyl-1,3-benzodiazole (1 g, 88%) as a light yellow solid. MS ESI calculated for C₉H₉BrN₂ [M+H]⁺, 224.99, 226.99; found 225.00, 227.00.

### Step 2: 1-ethyl-5-[2-(trimethylsilyl) ethynyl]-1,3-benzodiazole

To a stirred mixture of 5-bromo-1-ethyl-1,3-benzodiazole (1.00 g, 4.44 mmol), trimethylsilylacetylene (1.31 g, 13.33 mmol), CuI (0.17 g 0.89 mmol) and Pd(PPh₃)₂Cl₂ (0.62 g 0.89 mmol) in DMF (10.00 mL) was added TEA (8.99 g, 88.85 mmol). The reaction mixture was degassed with argon for three times and stirred for 16 h at 80° C. The resulting mixture was diluted with water (60 mL) and extracted with EtOAc (3×80 mL). The combined organic layers were washed with brine (150 mL), dried over anhydrous Na₂SO₄ and filtered. The filtrate was concentrated under reduced pressure. The residue was purified by silica gel column chromatography, eluted with CH₂Cl₂/MeOH (15:1). The fractions contained desired product were combined and concentrated to afford 1-ethyl-5-[2-(trimethylsilyl) ethynyl]-1,3-benzodiazole (0.80 g, 74%) as a light yellow solid. MS ESI calculated for C₁₄H₁₈N₂Si [M+H]⁺, 243.12; found 243.20.

### Step 3: 1-ethyl-5-ethynyl-1,3-benzodiazole

To a solution of 1-ethyl-5-[2-(trimethylsilyl)ethynyl]-1,3-benzodiazole (0.80 g, 3.30 mmol) in THF (8.00 mL) was added TBAF (1 M in THF, 4.95 mL, 4.95 mmol) at 0° C. under nitrogen atmosphere. The reaction mixture was stirred for 4 h at room temperature. The resulting mixture was concentrated under reduced pressure. The residue was purified by silica gel column chromatography, eluted with CH₂Cl₂/PE (15:1). The fractions contained desired product were combined and concentrated to afford 1-ethyl-5-ethynyl-1,3-benzodiazole (0.40 g, 71%) as a yellow solid. MS ESI calculated for C₁₁H₁₀N₂ [M+H]⁺, 171.08; found 171.15.

### Step 4: 3-[2-(1-ethyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide formic acid

To a stirred mixture of 3-bromo-1-[(3S,5R)-5-(methoxymethyl)-1-(prop-2-enoyl)pyrrolidin-3-yl]-5-(methylamino) pyrazole-4-carboxamide (0.20 g, 0.52 mmol), 1-ethyl-5-ethynyl-1,3-benzodiazole (0.13 g, 0.78 mmol), CuI (19.72 mg, 0.10 mmol) and Pd(PPh₃)₂Cl₂ (36.34 mg, 0.05 mmol) in DMF (3 mL) was added TEA (0.16 g, 1.55 mmol). The reaction mixture was degassed with argon for three times and stirred for 3 h at 90° C. The resulting mixture was concentrated under reduced pressure. The residue was purified by Prep-HPLC with the following conditions Column: Atlantis Prep T3 OBD Column, 19*250 mm 10 um; Mobile Phase A: Water (0.1% FA), Mobile Phase B: ACN; Flow rate: 20 mL/min; Gradient: 25 B to 60 B in 6 min; 210/254 nm; RT1: 5.88. The fractions contained desired product were combined and concentrated to afford 3-[2-(1-ethyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide formic acid (50.6 mg, 18%) as a light yellow solid. MS ESI calculated for C₂₆H₃₁N₇O₅ [M+H−FA]⁺, 476.23, found 476.15. ¹H-NMR (400 MHz, d₆-DMSO) δ 8.38 (s, 1H), 7.90 (s, 1H), 7.71 (d, J=8.4 Hz, 1H), 7.49-7.31 (m, 2H), 6.85-6.54 (m, 3H), 6.19-6.15 (m, 1H), 5.71-5.67 (m, 1H), 5.24 (dd, J=16.9, 7.5 Hz, 1H), 4.57-4.37 (m, 1H), 4.31 (q, J=7.3 Hz, 2H), 4.07-3.70 (m, 2H), 3.64-3.43 (m, 2H), 3.30 (s, 3H), 2.95 (t, J=5.2 Hz, 3H), 2.67-2.53 (m, 1H), 2.36-2.25 (m, 1H), 1.42 (t, J=7.2 Hz, 3H).

### Example 7: (S)-1-(1-acryloylpyrrolidin-3-yl)-3-((1,2-dimethyl-1H-benzo[d]imidazol-5-yl)ethynyl)-5-(methylamino)-1H-pyrazole-4-carboxamide

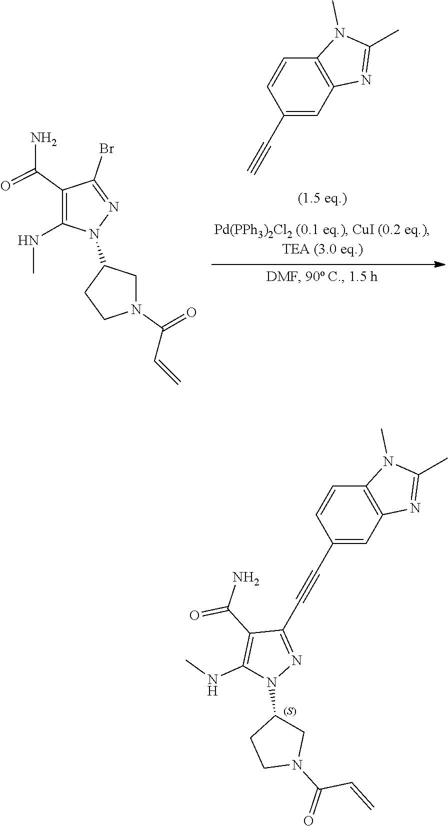

To a stirred mixture of 3-bromo-5-(methylamino)-1-[1-(prop-2-enoyl)pyrrolidin-3-yl]pyrazole-4-carboxamide (0.2 g, 0.58 mmol), 5-ethynyl-1,2-dimethyl-1,3-benzodiazole (0.15 g, 0.88 mmol), CuI (22.26 mg, 0.12 mmol) and Pd(PPh₃)₂Cl₂ (41.02 mg, 0.06 mmol) in DMF (2.00 mL) was added and TEA (0.24 mL, 2.41 mmol). The reaction mixture was degassed with argon for three times and stirred for 1.5 h at 90° C. The residue was purified by Prep-HPLC with the following conditions: Column: XBridge Prep C18 OBD Column, 19×150 mm 5 um; Mobile Phase A: Water (10 mmol/L NH₄HCO₃), Mobile Phase B: ACN; Flow rate: 20 mL/min; Gradient: 20 B to 50 B in 4.3 min; 210/254 nm; RT1: 4.02. The fractions contained desired product were combined and concentrated to afford 3-[2-(1,2-dimethyl-1,3-benzodiazol-5-yl)ethynyl]-5-(methylamino)-1-[(3S)-1-(prop-2-enoyl)pyrrolidin-3-yl]pyrazole-4-carboxamide (89 mg, 35%) as an off-white solid. MS ESI calculated for C₂₃H₂₅N₇O₂ [M+H]⁺, 431.21, found 431.20. H NMR (400 MHz, DMSO-d₆) δ 7.60 (s, 2H), 7.39-7.37 (d, J=8.3 Hz, 2H), 6.81 (s, 1H), 6.68-6.56 (m, 2H), 6.19-6.13 (m, 1H), 5.72-5.66 (m, 1H), 5.18-5.09 (m, 1H), 3.89-3.79 (m, 2H), 3.78-3.70 (m, 4H), 3.73 (m, 1H), 3.32 (s, 3H), 2.97-2.90 (m, 2H), 2.51-2.49 (m, 1H), 2.41-2.36 (m, 1H), 2.32-2.26 (m, 1H).

### Example 8: 1-((3S,5R)-1-acryloyl-5-(methoxymethyl)pyrrolidin-3-yl)-3-((1-methyl-1H-indazol-5-yl)ethynyl)-5-(methylamino)-1H-pyrazole-4-carboxamide

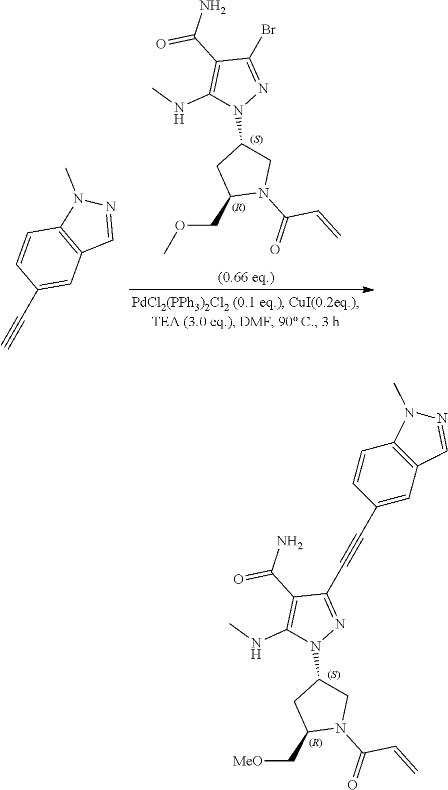

To a stirred mixture of 3-bromo-1-[(3S,5R)-5-(methoxymethyl)-1-(prop-2-enoyl)pyrrolidin-3-yl]-5-(methylamino) pyrazole-4-carboxamide (0.20 g, 0.52 mmol), 5-ethynyl-1-methylindazole (0.12 g, 0.78 mmol), CuI (19.72 mg, 0.10 mmol) and Pd(PPh₃)₂Cl₂ (36.34 mg, 0.05 mmol) in DMF (2.50 mL) was added TEA (0.16 g, 1.55 mmol). The reaction mixture was degassed with argon for three times and stirred for 3 h at 90° C. The reaction mixture was concentrated under reduced pressure. The residue was purified by Prep-HPLC with the following conditions Column: XBridge Prep C18 OBD Column, 19×150 mm 5 um; Mobile Phase A: Water (10 mmol/L NH₄HCO₃), Mobile Phase B: ACN; Flow rate: 20 mL/min; Gradient: 30 B to 60 B in 4.3 min; 254/210 nm; RT1:4.350. The fractions contained desired product were combined and concentrated to afford 1-[(3S,5R)-5-(methoxymethyl)-1-(prop-2-enoyl)pyrrolidin-3-yl]-5-(methylamino)-3-[2-(1-methylindazol-5-yl)ethynyl]pyrazole-4-carboxamide (80.0 mg, 33%) as a white solid. MS ESI calculated for C₂₄H₂₇N₇O₃ [M+H]⁺, 462.22, found 462.05. ¹H-NMR (300 MHz, d₆-DMSO) δ 8.10 (d, J=21.4 Hz, 2H), 7.74 (d, J=8.8 Hz, 1H), 7.55 (dd, J=8.7, 1.7 Hz, 1H), 7.36 (s, 1H), 6.89-6.52 (m, 3H), 6.18 (d, J=16.6 Hz, 1H), 5.71 (d, J=10.3 Hz, 1H), 5.27 (dd, J=13.6, 6.9 Hz, 1H), 4.48 (d, J=39.4 Hz, 1H), 4.14-4.00 (m, 4H), 3.96-3.84 (m, 1H), 3.81-3.57 (m, 1H), 3.50-3.48 (m, 1H), 3.37-3.29 (m, 4H), 2.96 (t, J=4.5 Hz, 3H), 2.33-2.30 (m, 1H).

### Example 9: 1-((3S,5R)-1-acryloyl-5-(methoxymethyl)pyrrolidin-3-yl)-3-((2-methyl-2H-indazol-5-yl)ethynyl)-5-(methylamino)-1H-pyrazole-4-carboxamide

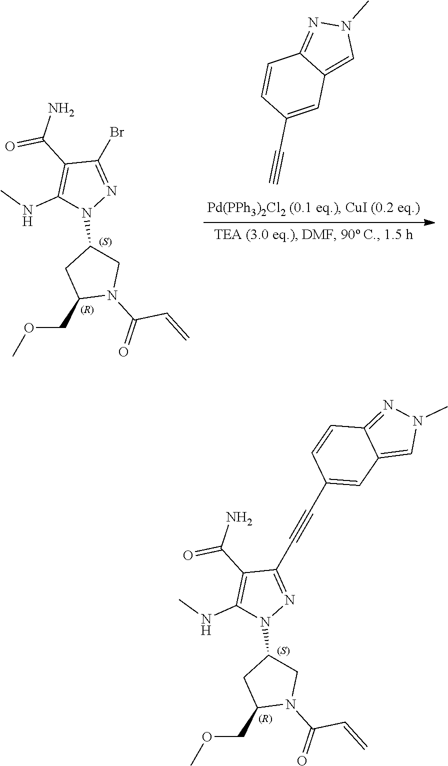

To a stirred solution of 3-bromo-1-[(3S,5R)-5-(methoxymethyl)-1-(prop-2-enoyl)pyrrolidin-3-yl]-5-(methylamino)pyrazole-4-carboxamide (0.30 g, 0.78 mmol) in DMF (5.00 mL) were added 5-ethynyl-2-methylindazole (0.18 g, 1.17 mmol), palladium chloride; bis(triphenylphosphine) (54.52 mg, 0.08 mmol), copper (I) iodide (29.58 mg, 0.16 mmol) and TEA (0.24 g, 2.33 mmol). The reaction mixture was degassed with argon for three times and stirred for 1.5 h at 90° C. The resulting mixture was directly purified by reverse phase chromatography with the following conditions: Column: Spherical C18, 20-40 um, 80 g; Mobile Phase A: Water (10 mM NH₄HCO₃), Mobile Phase B: ACN; Flow rate: 40 m/min; Gradient (B %): 0% hold 5 min, 0%-23% within 20 min, 23% hold 5 min, 23%-31% within 15 min, 31% hold 6 min, 31%-95% within 5 min, 95% hold 3 min; Detector: UV 254 & 220 nm; RT: 45 min. The fractions contained desired product were combined and concentrated under reduced pressure. The residue was further purified by Prep-HPLC with the following conditions: Column: XBridge Prep C18 OBD Column, 19×150 mm, 5 um; Mobile Phase A: Water (10 mM NH₄HCO₃), Mobile Phase B: ACN; Flow rate: 20 mL/min; Gradient: 20% B to 50% B in 4.3 min; Detector: UV 210 & 254 nm; RT: 4.02 min. The fractions contained desired product were combined and concentrated under reduced pressure to afford 1-[(3S,5R)-5-(methoxymethyl)-1-(prop-2-enoyl)pyrrolidin-3-yl]-5-(methylamino)-3-[2-(2-methylindazol-5-yl)ethynyl]pyrazole-4-carboxamide (74.0 mg, 20%) as a white solid. MS ESI calculated for C₂₄H₂₇N₇O₃ [M+H]⁺, 462.22, found 462.10. H-NMR (300 MHz, d₆-DMSO) δ 8.44 (s, 1H), 8.02 (s, 1H), 7.64 (d, J=8.9 Hz, 1H), 7.33 (brs, 1H), 7.31 (dd, J=8.9, 1.6 Hz, 1H), 6.80 (brs, 1H), 6.76-6.54 (m, 2H), 6.20-6.13 (m, 1H), 5.71-5.66 (m, 1H), 5.31-5.17 (m, 1H), 4.56-4.38 (m, 1H), 4.19 (s, 3H), 4.05-3.70 (m, 2H), 3.63-3.43 (m, 2H), 3.29 (s, 3H), 2.94-2.92 (m, 3H), 2.66-2.56 (m, 0.5H), 2.47-2.42 (m, 0.5H), 2.33-2.25 (m, 1H).

### Example 10: 1-((3S,5R)-1-acryloyl-5-(methoxymethyl)pyrrolidin-3-yl)-3-((1-methyl-2-(trifluoromethyl)-1H-benzo[d]imidazol-5-yl)ethynyl)-5-(methylamino)-1H-pyrazole-4-carboxamide

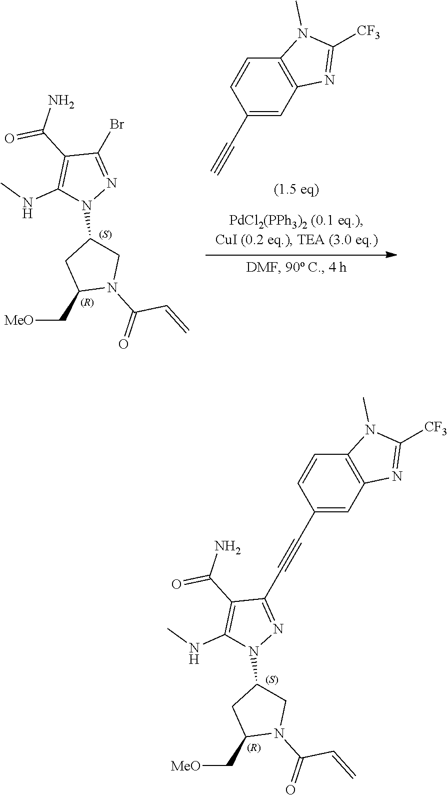

To a mixture of 3-bromo-1-[(3S,5R)-5-(methoxymethyl)-1-(prop-2-enoyl)pyrrolidin-3-yl]-5-(methylamino)pyrazole-4-carboxamide (0.86 g, 2.23 mmol), 5-ethynyl-1-methyl-2-(trifluoromethyl)-1,3-benzodiazole (1.00 g, 4.45 mmol), Pd(PPh₃)₂Cl₂ (0.16 g, 0.22 mmol) and CuI (84.81 mg, 0.45 mmol) in DMF (9.00 mL) was added TEA (0.93 mL, 9.17 mmol). The reaction mixture was degassed with nitrogen for three times and stirred for 4 h at 90° C. The resulting mixture was purified by reverse flash chromatography with the following conditions: column, C18 silica gel; mobile phase, NH₄HCO₃ in water, 10% to 50% gradient in 25 min; detector, UV 254 nm. The fractions contained desired product were combined and concentrated. The residue was purified by Prep-HPLC with the following conditions Column: XBridge Prep OBD C18 Column, 19*250 mm, 5 um; Mobile Phase A: Water (10 mmol/L NH₄HCO₃), Mobile Phase B: ACN; Flow rate: 20 mL/min; Gradient: 30 B to 50 B in 5.8 min; 210/254 nm; RT1: 5.58. The fractions contained desired product were combined and concentrated to afford 1-[(3S,5R)-5-(methoxymethyl)-1-(prop-2-enoyl)pyrrolidin-3-yl]-3-[2-[1-methyl-2-(trifluoromethyl)-1,3-benzodiazol-5-yl]ethynyl]-5-(methylamino)pyrazole-4-carboxamide (0.37 g, 30%) as an off-white solid. MS ESI calculated for C₂₅H₂₆F₃N₇O₃[M+H]⁺, 530.20, found 530.15. H-NMR (400 MHz, DMSO-d₆): δ 8.09 (d, J=1.6 Hz, 1H), 7.89 (d, J=8.6 Hz, 1H), 7.67 (dd, J=8.5, 1.5 Hz, 1H), 7.35 (s, 1H), 6.88-6.47 (m, 3H), 6.23-6.18 (m, 1H), 5.71-5.68 (m, 1H), 5.27-5.24 (m, 1H), 4.52-4.41 (m, 1H), 4.02-4.00 (m, 4H), 3.93-3.82 (m, 1H), 3.63-3.42 (m, 2H), 3.31 (d, J=5.4 Hz, 3H), 2.94 (t, J=5.1 Hz, 3H), 2.63-260 (m, 1H), 2.34-2.24 (m, 1H).

### Example 11: 1-((3S,5R)-1-acryloyl-5-(methoxymethyl)pyrrolidin-3-yl)-3-((4,6-difluoro-1-methyl-1H-benzo[d]imidazol-5-yl)ethynyl)-5-(methylamino)-1H-pyrazole-4-carboxamide

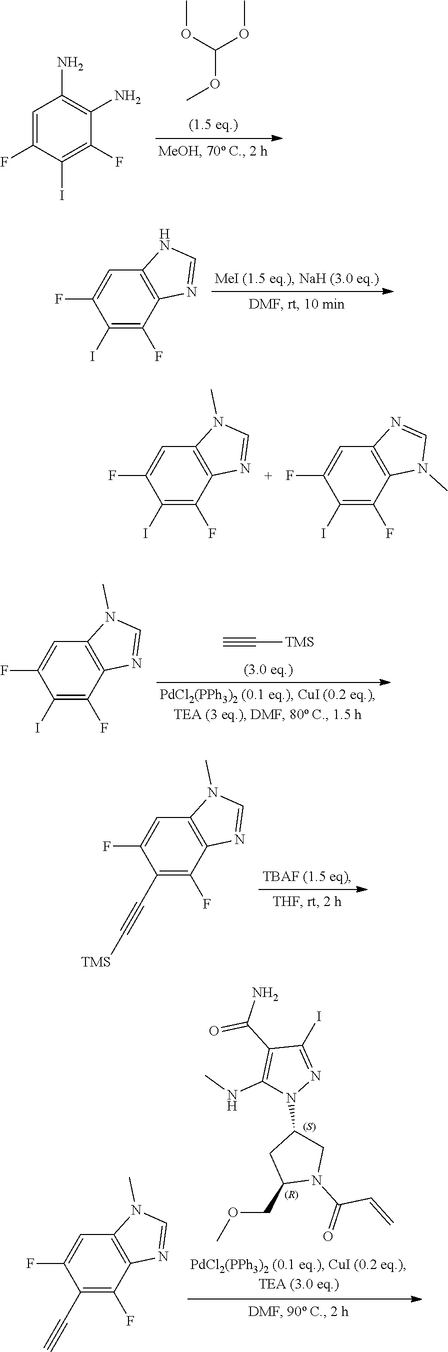

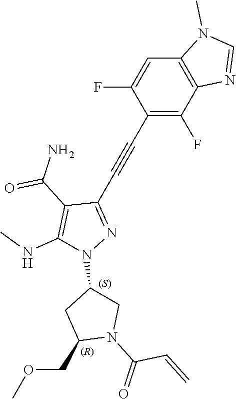

### Step 1: 4,6-difluoro-5-iodo-1H-1,3-benzodiazole

To a solution of 3,5-difluoro-4-iodobenzene-1,2-diamine (5.00 g, 18.51 mmol) in MeOH (50.00 mL) was added trimethyl orthoformate (2.94 g, 27.70 mmol) at room temperature. The reaction mixture was stirred for 2 h at 70° C. The resulting mixture was concentrated under reduced pressure. The resulting mixture was diluted with EtOAc (50.00 mL). The residue was washed with water (3×40.00 mL). The combined organic layers was dried over anhydrous Na₂SO₄ and filtered. The filtrate was concentrated under reduced pressure. The residue was purified by silica gel column chromatography, eluted with EA in PE (0-60%). The fractions contained desired product were combined and concentrated to afford 4,6-difluoro-5-iodo-1H-1,3-benzodiazole (4.9 g, 94%) as a brown solid. MS ESI calculated for C₇H₃F₂IN₂ [M+H]⁺, 280.93, found 280.95.

### Step 2: 4,6-difluoro-5-iodo-1-methyl-1,3-benzodiazole

To a solution of 4,6-difluoro-5-iodo-1H-1,3-benzodiazole (0.30 g, 1.07 mmol) in DMF (12.00 mL) was added NaH (0.13 g, 3.21 mmol, 60%) at 0° C. under nitrogen atmosphere. The reaction mixture was stirred at ambient temperature for 30 min. methyl iodide (0.23 g, 1.60 mmol) was added dropwise at 0° C. under nitrogen atmosphere. The resulting mixture was stirred for 10 min at room temperature. The resulting mixture was quenched with Water (1.00 mL) at 0° C. The resulting mixture was diluted with EtOAc (20.00 mL). The residue was washed with water (3×10.00 mL). The combined organic layers were washed with brine (20 mL), dried over anhydrous Na₂SO₄. After filtration, the filtrate was concentrated under reduced pressure. The residue was purified by reverse phase chromatography with the following conditions: Column: Spherical C18, 20-40 um, 40 g; Mobile Phase A: Water (10 mM NH₄HCO₃), Mobile Phase B: ACN; Flow rate: 50 mL/min; Gradient (B %): 5% hold 5 min, 5%-29% within 25 min; 29% hold 8 min, 29˜95% within 5 min, 95% hold 5 min; Detector: UV 254 & 220 nm; RT: 48 min. The fractions contained desired product were combined and concentrated under reduced pressure to afford 4,6-difluoro-5-iodo-1-methyl-1,3-benzodiazole (0.18 g, 57%) as a light yellow solid and 5,7-difluoro-6-iodo-1-methyl-1,3-benzodiazole (0.15 g, 47%) as a yellow solid. MS ESI calculated for C₈H₅F₂IN₂ [M+H]⁺, 294.95, found 295.00.

### Step 3: 4,6-difluoro-1-methyl-5-[2-(trimethylsilyl)ethynyl]-1,3-benzodiazole

Into a 50 mL vial were added 4,6-difluoro-5-iodo-1-methyl-1,3-benzodiazole (1.60 g, 5.44 mmol), trimethylsilylacetylene (1.60 g, 16.32 mmol), Pd(PPh₃)₂Cl₂ (0.38 g, 0.54 mmol), CuI (0.21 g, 1.09 mmol) and TEA (1.65 g, 16.32 mmol) in DMF (20.00 mL) at room temperature. The reaction mixture was degassed with argon for three times and stirred for 2 h at 80° C. The mixture was allowed to cool down to room temperature. The resulting mixture was concentrated under reduced pressure. The resulting mixture was extracted with EtOAc (3×100 mL). The combined organic layers were washed with brine (3×100 mL), dried over anhydrous Na₂SO₄ and filtered. The filtrate was concentrated under reduced pressure. The residue was purified by silica gel column chromatography, eluted with PE/EtOAc (1:9). The fractions contained desired product were combined and concentrated to afford 4,6-difluoro-1-methyl-5-[2-(trimethylsilyl)ethynyl]-1,3-benzodiazole 4,6-difluoro-1-methyl-5-[2-(trimethylsilyl)ethynyl]-1,3-benzodiazole (1.2 g, 83%) as a yellow solid. MS ESI calculated for C₁₃H₁₄F₂N₂Si [M+H]⁺, 264.08, found 265.35.

### Step 4: 5-ethynyl-4,6-difluoro-1-methyl-1,3-benzodiazole

To a stirred solution of 4,6-difluoro-1-methyl-5-[2-(trimethylsilyl)ethynyl]-1,3-benzodiazole (1.30 g, 4.92 mmol) in THF (10.00 mL) was added TBAF (1.93 g, 7.38 mmol) dropwise at room temperature. The resulting mixture was stirred for 2 h at room temperature. The resulting mixture was extracted with EtOAc (3×200 mL). The combined organic layers were washed with brine (3×50 mL), dried over anhydrous Na₂SO₄ and filtered. The filtrate was concentrated under reduced pressure. The residue was purified by silica gel column chromatography, eluted with PE/EtOAc (1:9). The fractions contained desired product were combined and concentrated to afford 5-ethynyl-4,6-difluoro-1-methyl-1,3-benzodiazole (0.90 g, 95%) as a yellow solid. MS ESI calculated for C₁₀H₆F₂N₂ [M+H]⁺, 192.04, found 193.16.

### Step 5: 3-[2-(4,6-difluoro-1-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

Into a 50 mL vial were added 3-iodo-1-[(3S,5R)-5-(methoxymethyl)-1-(prop-2-enoyl)pyrrolidin-3-yl]-5-(methylamino)pyrazole-4-carboxamide (0.70 g, 1.62 mmol), 5-ethynyl-4,6-difluoro-1-methyl-1,3-benzodiazole (0.37 g, 1.94 mmol), Pd(PPh₃)₂Cl₂ (0.11 g, 0.16 mmol), CuI (61.54 mg, 0.32 mmol) and TEA (0.49 g, 4.85 mmol) in DMF (10.00 mL) at room temperature. The reaction mixture was degassed with argon for three times and stirred for 2 h at 90° C. The mixture was allowed to cool down to room temperature. The resulting mixture was concentrated under reduced pressure. The residue was purified by silica gel column chromatography, eluted with CH₂Cl₂/MeOH (0-6%). The fractions contained desired product were combined and concentrated. The residue was purified by reverse phase flash chromatography with the following conditions: Column: Spherical C18, 20-40 um, 330 g; Mobile Phase A: Water (plus 3.2 g NH₄HCO₃); Mobile Phase B: ACN; Flow rate: 90 mL/min; Gradient: 5%-5% B, 10 min, 25% B-50% B gradient in 20 min; Detector: 254 nm. The fractions contained the desired product were collected at 44% B and concentrated under reduced pressure to afford 3-[2-(4,6-difluoro-1-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (0.51 g, 62%) as a white solid. MS ESI calculated for C₂₄H₂₅F₂N₇O₃ [M+H]⁺, 497.19, found 498.50. 1H-NMR (400 MHz, DMSO-d₆) δ 8.37 (s, 1H), 7.70-7.48 (m, 2H), 6.87-6.53 (m, 3H), 6.13-6.19 (m, 1H), 5.71-5.68 (m, 1H), 5.28-5.25 (m, 1H), 4.46 (dd, J=56.9, 4.3 Hz, 1H), 4.07-3.71 (m, 5H), 3.65-3.42 (m, 2H), 3.31 (d, J=5.5 Hz, 3H), 2.97 (t, J=5.4 Hz, 3H), 2.65-2.62 (m, 1H), 2.32-2.29 (m, 1H).

### Example 12: 1-((3S,5R)-1-acryloyl-5-(methoxymethyl)pyrrolidin-3-yl)-3-((1-cyclopropyl-2-methyl-1H-benzo[d]imidazol-5-yl)ethynyl)-5-(methylamino)-1H-pyrazole-4-carboxamide

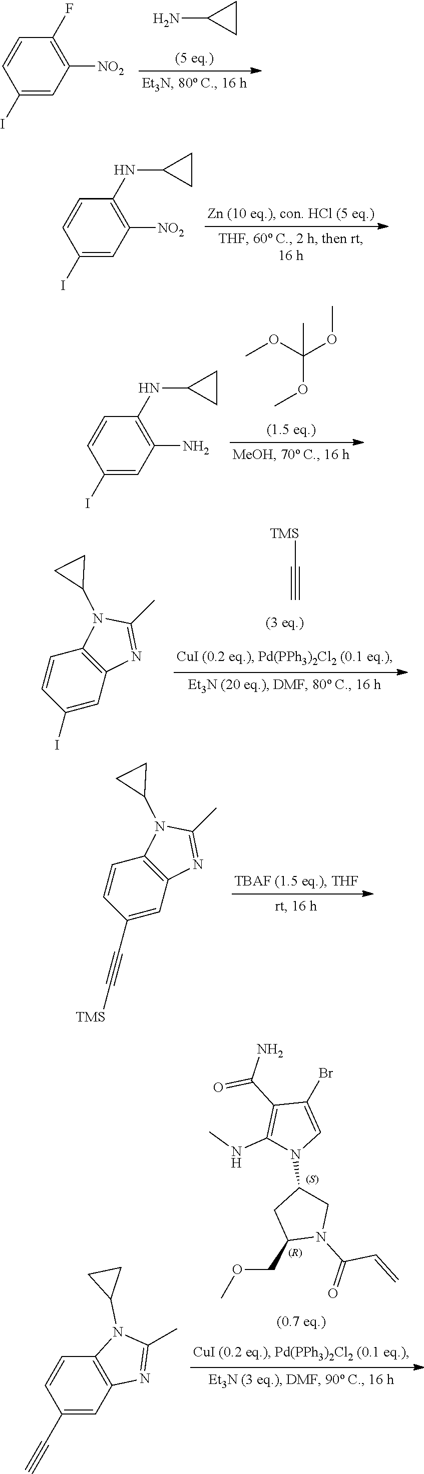

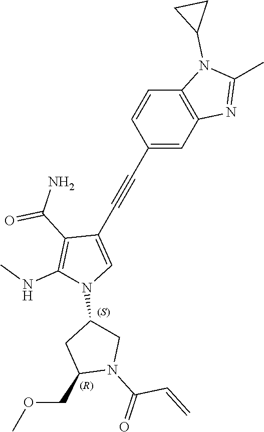

### Step 1: N-cyclopropyl-4-iodo-2-nitroaniline

To a stirred mixture of 1-fluoro-4-iodo-2-nitrobenzene (10.00 g, 37.453 mmol) in Et₃N (29.15 mL, 288.10 mmol) was added aminocyclopropane (10.69 g, 187.27 mmol) at room temperature. The reaction mixture was stirred for 16 h at 80° C. under nitrogen atmosphere. The mixture was allowed to cool down to room temperature. The resulting mixture was quenched by the addition of sat. NaHCO₃ (aq.) (300 mL), extracted with EtOAc (3×200 mL). The combined organic layers were washed with brine (300 mL), dried over anhydrous Na₂SO₄ and filtered. The filtrate was concentrated under reduced pressure to afford N-cyclopropyl-4-iodo-2-nitroaniline (10 g, 87%) as a brown solid which was used in the next step without further purification. MS ESI calculated for C₉H₉IN₂O₂[M+H]+, 304.00, found 305.00.

### Step 2: N1-cyclopropyl-4-iodobenzene-1,2-diamine

To a stirred mixture of N-cyclopropyl-4-iodo-2-nitroaniline (5.20 g, 17.10 mmol) in THF (1.00 mL) was added Zn (11.19 g, 171.00 mmol) and HCl (2.60 mL, 71.25 mmol) at room temperature. The reaction mixture was stirred for 2 h at 60° C., then 16 h at room temperature. The resulting mixture was filtered, the filter cake was washed with EtOAc (3×300 mL). The filtrate was concentrated under reduced pressure and dried to afford N¹-cyclopropyl-4-iodobenzene-1,2-diamine (5 g, crude) as a brown solid which was used in the next step without further purification. MS ESI calculated for C₉H₁₁IN₂ [M+H]⁺, 274.95, found 274.95.

### Step 3: 1-cyclopropyl-5-iodo-2-methyl-1,3-benzodiazole

To a stirred mixture of N¹-cyclopropyl-4-iodobenzene-1,2-diamine (0.20 g, 0.73 mmol) in methanol (2.00 mL) was added 1,1,1-trimethoxyethane (0.13 g, 1.09 mmol) at room temperature. The reaction mixture was stirred for 16 h at 70° C. The resulting mixture was allowed to cool down to room temperature. The resulting mixture was concentrated under reduced pressure. The residue was purified by silica gel column chromatography, eluted with PE/(EtOAc/EtOH(3:1)) (1:1). The fractions contained desired product were combined and concentrated to afford 1-cyclopropyl-5-iodo-2-methyl-1,3-benzodiazole (1.4 g, 51%) as a yellow solid. MS ESI calculated for C₁₁H₁₁IN₂ [M+H]⁺, 298.95, found 298.95.

### Step 4: 1-cyclopropyl-2-methyl-5-[2-(trimethylsilyl)ethynyl]-1,3-benzodiazole

To a stirred mixture of 1-cyclopropyl-5-iodo-2-methyl-1,3-benzodiazole (0.70 g, 2.35 mmol) and CuI (89.44 mg, 0.47 mmol) in DMF (7.00 mL) were added Pd(PPh₃)₂Cl₂ (0.16 g, 0.24 mmol), trimethylsilylacetylene (98.84 mg, 1.01 mmol) and TEA (6.53 mL, 64.51 mmol). The reaction mixture was degassed with nitrogen for three times and stirred for 16 h at 80° C. The mixture was allowed to cool down to room temperature. The resulting mixture was quenched by the addition of Water (50 mL) at room temperature, extracted with EtOAc (3×50 mL). The combined organic layers were washed with brine (100 mL), dried over anhydrous Na₂SO₄ and filtered. The filtrate was concentrated under reduced pressure. The residue was purified by silica gel column chromatography, eluted with PE/(EtOAc/EtOH(3:1)) (1:1). The fractions contained desired product were combined and concentrated to afford 1-cyclopropyl-2-methyl-5-[2-(trimethylsilyl)ethynyl]-1,3-benzodiazole (0.50 g, 79%) as a brown solid. MS ESI calculated for C₁₆H₂₀N₂Si [M+H]⁺, 269.10, found 269.10.

### Step 5: 1-cyclopropyl-5-ethynyl-2-methyl-1,3-benzodiazole

To a stirred solution of 1-cyclopropyl-2-methyl-5-[2-(trimethylsilyl)ethynyl]-1,3-benzodiazole (0.50 g, 1.86 mmol) in THF (5.00 mL) was added TBAF (2.79 mL, 2.79 mmol) at 0° C. The reaction mixture was stirred for 16 h at room temperature. The resulting mixture was quenched by the addition of Water (50 mL) at room temperature. The resulting mixture was extracted with EtOAc (3×50 mL). The combined organic layers were washed with brine (3×50 mL), dried over anhydrous Na₂SO₄ and filtered. The filtrate was concentrated under reduced pressure. The residue was purified by silica gel column chromatography, eluted with PE/EtOAc (1:4). The fractions contained desired product were combined and concentrated to afford 1-cyclopropyl-5-ethynyl-2-methyl-1,3-benzodiazole (0.28 g, 76%) as a yellow solid. MS ESI calculated for C₁₃H₁₂N₂[M+H]⁺, 197.05, found 197.05.

### Step 6: 3-[2-(1-cyclopropyl-2-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a stirred mixture of 3-bromo-1-[(3S,5R)-5-(methoxymethyl)-1-(prop-2-enoyl)pyrrolidin-3-yl]-5-(methylamino)pyrazole-4-carboxamide (0.17 g, 0.44 mmol) and 1-cyclopropyl-5-ethynyl-2-methyl-1,3-benzodiazole (0.13 g, 0.66 mmol) in DMF (2.00 mL) were added CuI (16.76 mg, 0.09 mmol), TEA (0.18 mL, 1.81 mmol) and Pd(PPh₃)₂Cl₂ (30.89 mg, 0.04 mmol) at room temperature. The reaction mixture was degassed with argon for three times and stirred for 16 h at 90° C. The mixture was allowed to cool down to room temperature. The residue was purified by reverse flash chromatography with the following conditions: column, C18 silica gel; mobile phase, ACN in water (0.1% NH₄HCO₃), 10% to 50% gradient in 20 min; detector, UV 254 nm to give crude product. Then the crude product was further purified by Prep-HPLC with the following conditions: Column: XBridge Prep C18 OBD Column, 19×150 mm 5 um; Mobile Phase A: Water (10 mM NH₄HCO₃), Mobile Phase B: ACN; Flow rate: 20 mL/min; Gradient: 40 B to 80 B in 4.3 min; 210/254 nm; RT1:4.02. The fractions contained desired product were combined and concentrated to afford 3-[2-(1-cyclopropyl-2-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (72.5 mg, 32%) as an off-white solid. MS ESI calculated for C₂₇H₃₁N₇O₃ [M+H]⁺, 501.15, found 502.1; ¹H NMR (400 MHz, DMSO-d₆) δ 7.76 (s, 1H), 7.61 (d, J=8.4 Hz, 1H), 7.44-7.35 (m, 2H), 6.91-6.58 (m, 2H), 6.21-6.18 (m, 1H), 5.69 (dd, J=10.3, 2.7 Hz, 1H), 5.29-5.18 (m, 1H), 4.56-4.36 (m, 1H), 4.10-4.01 (m, 1H), 3.91-3.83 (m, 1H), 3.61-3.58 (m, 1H), 3.51-3.43 (m, 2H), 3.12-3.02 (m, 3H), 2.94 (s, 3H), 2.62 (s, 3H), 2.48-2.43 (m, 1H), 2.36-2.27 (s, 2H), 1.25-1.14 (m, 2H), 1.13-0.97 (m, 2H).

### Example 13: 1-((3S,5R)-1-acryloyl-5-(methoxymethyl)pyrrolidin-3-yl)-3-((1-cyclopropyl-1H-benzo[d]imidazol-5-yl)ethynyl)-5-(methylamino)-1H-pyrazole-4-carboxamide

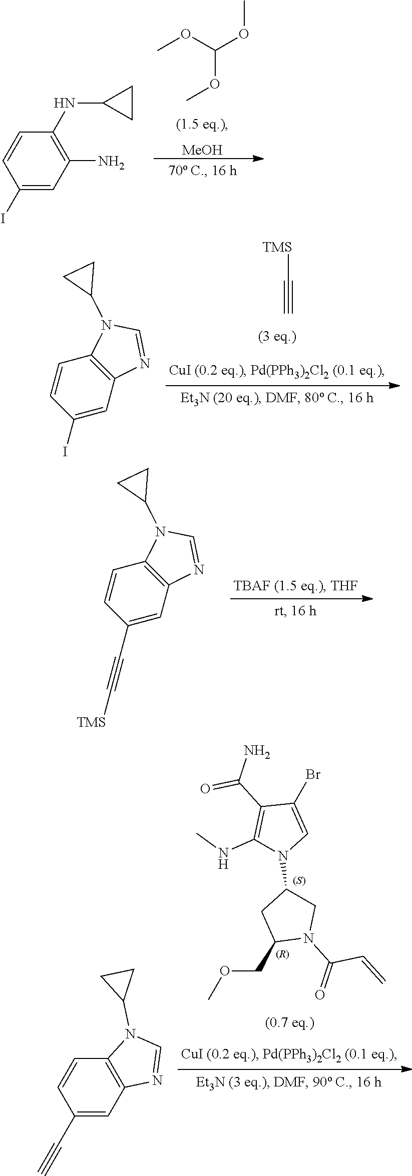

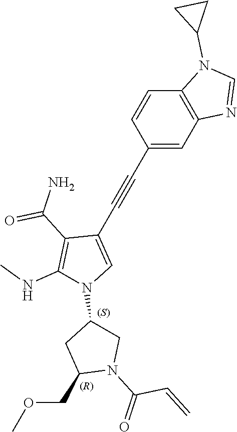

### Step 1: 1-cyclopropyl-5-iodo-1,3-benzodiazole

To a stirred mixture of N-cyclopropyl-4-iodobenzene-1,2-diamine (2.50 g, 9.12 mmol) in methanol (30.00 mL) was added trimethyl orthoformate (1.45 g, 13.68 mmol) at room temperature. The reaction mixture was stirred for 16 h at 70° C. The mixture was allowed to cool down to room temperature. The resulting mixture was concentrated under reduced pressure. The residue was purified by silica gel column chromatography, eluted with PE/(EtOAc/EtOH) (3:1)) (1:1). The fractions contained desired product were combined and concentrated to afford 1-cyclopropyl-5-iodo-1,3-benzodiazole (1.2 g, 46%) as a yellow solid. MS ESI calculated for C₁₀H₉IN₂ [M+H]⁺, 284.95, found 284.95.

### Step 2: 1-cyclopropyl-5-[2-(trimethylsilyl)ethynyl]-1,3-benzodiazole

To a stirred mixture of 1-cyclopropyl-5-iodo-1,3-benzodiazole (0.70 g, 2.46 mmol) and CuI (93.85 mg, 0.49 mmol) in DMF (7.00 mL) were added Pd(PPh3)2Cl2 (0.17 g 0.25 mmol), trimethylsilylacetylene (0.10 g, 1.06 mmol) and TEA (6.85 mL, 67.69 mmol) at room temperature. The reaction mixture was degassed with argon for three times and stirred for 16 h at 80° C. The reaction was quenched by the addition of Water (50 mL) at room temperature, extracted with EtOAc (3×50 mL). The combined organic layers were washed with brine (150 mL), dried over anhydrous Na₂SO₄ and filtered. The filtrate was concentrated under reduced pressure. The residue was purified by silica gel column chromatography, eluted with PE/(EtOAc/EtOH (3:1)) (1:1). The fractions contained desired product were combined and concentrated to afford 1-cyclopropyl-5-[2-(trimethylsilyl)ethynyl]-1,3-benzodiazole (0.58 g, 92%) as a brown oil. MS ESI calculated for C₁₅H₁₈N₂Si [M+H]⁺, 255.15, found 255.15.

### Step 3: 1-cyclopropyl-5-ethynyl-1,3-benzodiazole

To a stirred solution of 1-cyclopropyl-5-[2-(trimethylsilyl)ethynyl]-1,3-benzodiazole (0.60 g, 2.36 mmol) in THF (6.00 mL) was added TBAF (3.54 mL, 3.54 mmol) at 0° C. The reaction mixture was stirred for 16 h at room temperature. The resulting mixture was quenched by the addition of Water (70 mL) at room temperature, extracted with EtOAc (3×70 mL). The combined organic layers were washed with brine (150 mL), dried over anhydrous Na₂SO₄ and filtered. The filtrate was concentrated under reduced pressure. The residue was purified by silica gel column chromatography, eluted with PE/EtOAc (1:4). The fractions contained desired product were combined and concentrated to afford 1-cyclopropyl-5-ethynyl-1,3-benzodiazole (0.26 g, 60%) as a yellow oil. MS ESI calculated for C₁₀H₁₂N₂[M+H]⁺, 183.05, found 183.05.

### Step 4: 3-[2-(1-cyclopropyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a stirred mixture of 3-bromo-1-[(3S,5R)-5-(methoxymethyl)-1-(prop-2-enoyl)pyrrolidin-3-yl]-5-(methylamino)pyrazole-4-carboxamide (0.17 g, 0.44 mmol) and 1-cyclopropyl-5-ethynyl-1,3-benzodiazole (0.12 g, 0.66 mmol) in DMF (2.00 mL) were added CuI (16.76 mg, 0.09 mmol), TEA (0.18 mL, 1.81 mmol) and Pd(PPh₃)₂Cl₂ (30.89 mg, 0.04 mmol) at room temperature. The reaction mixture was degassed with argon for three times and stirred for 16 h at 90° C. The resulting mixture was allowed to cool down to room temperature. The residue was purified by reverse flash chromatography with the following conditions: column, C18 silica gel; mobile phase, ACN in water (0.1% NH₄HCO₃), 10% to 50% gradient in 20 min; detector, UV 254 nm. The fractions contained desired product were combined and concentrated. The residue was purified by reverse flash chromatography with the following conditions: Column: XBridge Prep C18 OBD Column, 19×150 mm 5 um; Mobile Phase A: Water (10 mmol/L NH₄HCO₃), Mobile Phase B: ACN; Flow rate: 20 mL/min; Gradient: 40 B to 80 B in 4.3 min; 210/254 nm; RT1: 4.02. The fractions contained desired product were combined and concentrated to afford 3-[2-(1-cyclopropyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (74.2 mg, 34%) as an off-white solid. MS ESI calculated for C₂₆H₂₉N₇O₃ [M+H]⁺, 488.10, found 488.10. H-NMR (400 MHz, DMSO-d₆) δ 8.36 (s, 1H), 7.90 (s, 1H), 7.70 (d, J=8.3 Hz, 1H), 7.49 (d, J=8.7 Hz, 1H), 7.37 (s, 1H), 6.85-6.52 (m, 2H), 6.17 (dd, J=16.6, 2.6 Hz, 1H), 5.69 (dd, J=10.2, 2.6 Hz, 1H), 5.33-5.18 (m, 1H), 4.42-4.38 (m, 1H), 4.07-3.98 (m, 1H), 3.92-3.83 (m, 1H), 3.65-3.51 (m, 2H), 3.49-3.42-3.38 (m, 1H), 3.31-3.30 (m, 3H), 2.94 (t, J=5.1 Hz, 3H), 2.53-2.48 (m, 2H), 2.35-2.31 (m, 1H), 1.16-1.01 (m, 4H).

### Example 14: 1-((3S,5R)-1-acryloyl-5-(methoxymethyl)pyrrolidin-3-yl)-3-((1-ethyl-1H-indazol-5-yl)ethynyl)-5-(methylamino)-1H-pyrazole-4-carboxamide

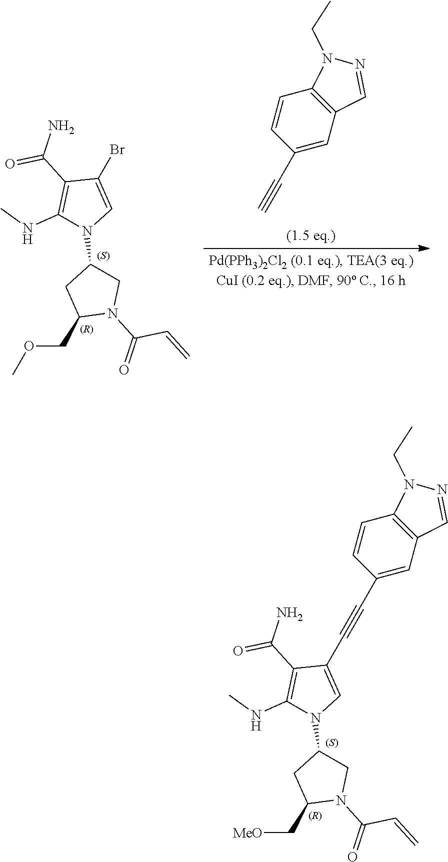

To a stirred mixture of 3-bromo-1-[(3S,5R)-5-(methoxymethyl)-1-(prop-2-enoyl)pyrrolidin-3-yl]-5-(methylamino)pyrazole-4-carboxamide (0.18 g, 0.47 mmol), 1-ethyl-5-ethynylindazole (0.12 g, 0.70 mmol), CuI (17.75 mg, 0.10 mmol) and Pd(PPh₃)₂Cl₂ (32.71 mg, 0.05 mmol) in DMF (3.00 mL) was added TEA (0.19 mL, 1.92 mmol). The reaction mixture was degassed with nitrogen for three times and stirred for 16 h at 90° C. The resulting mixture was purified by reverse flash chromatography with the following conditions: column, C18 silica gel; mobile phase, NH₄HCO₃ in water, 25% to 40% gradient in 25 min; detector, UV 254 nm. The crude product was purified by Prep-HPLC with the following conditions Column: XBridge Prep C18 OBD Column, 19×150 mm 5 um; Mobile Phase A: Water (10 mmol/L NH₄HCO₃), Mobile Phase B: ACN; Flow rate: 20 mL/min; Gradient: 40 B to 60 B in 4.3 min; 210/254 nm; RT1:4.12. The fractions contained desired product were combined and concentrated to afford 3-[2-(1-ethylindazol-5-yl)ethynyl]-1-[(3S,5R)-5-(methoxymethyl)-1-(prop-2-enoyl)pyrrolidin-3-yl]-5-(methylamino)pyrazole-4-carboxamide (57.5 mg, 25%) as an off-white solid. MS ESI calculated for C₂₅H₂₉N₇O₃ [M+H]⁺, 476.23, found 476.25. H-NMR (400 MHz, DMSO-d₆): δ 8.15-8.10 (m, 2H), 7.78 (d, J=8.6 Hz, 1H), 7.59-7.50 (m, 1H), 7.36 (s, 1H), 6.90-6.49 (m, 3H), 6.18 (d, J=16.6 Hz, 1H), 5.71 (d, J=10.4 Hz, 1H), 5.26 (s, 1H), 4.52-4.45 (m, 3H), 4.09-3.68 (m, 2H), 3.65-3.37 (m, 2H), 3.33-3.31 (m, 3H), 2.96 (t, J=4.5 Hz, 3H), 2.55-2.53 (m, 1H), 2.34-2.32 (m, 1H), 1.45-1.41 (m, 3H).

### Example 15: 1-((3S,5R)-1-acryloyl-5-(methoxymethyl)pyrrolidin-3-yl)-3-((2-ethyl-2H-indazol-5-yl)ethynyl)-5-(methylamino)-1H-pyrazole-4-carboxamide

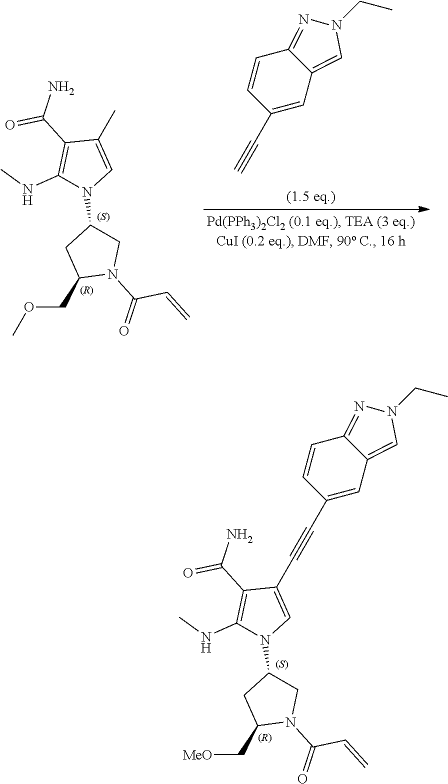

To a stirred mixture of 3-bromo-1-[(3S,5R)-5-(methoxymethyl)-1-(prop-2-enoyl)pyrrolidin-3-yl]-5-(methylamino)pyrazole-4-carboxamide (0.25 g, 0.65 mmol), 2-ethyl-5-ethynylindazole (0.16 g, 0.97 mmol), CuI (24.65 mg, 0.13 mmol) and Pd(PPh₃)₂Cl₂ (45.43 mg, 0.06 mmol) in DMF (2.50 mL) was added TEA (0.27 mL, 2.67 mmol). The reaction mixture was degassed with nitrogen for three times and stirred for 16 h at 90° C. The resulting mixture was diluted with water (40 mL) and extracted with EA (3×50 mL). The combined organic layers were washed with brine (2×100 mL), dried over anhydrous Na₂SO₄, filtered and concentrated under reduced pressure. The residue was purified by reverse flash chromatography with the following conditions: column, C18 silica gel; mobile phase, NH₄HCO₃ in water, 25% to 40% gradient in 25 min; detector, UV 254 nm. The fractions contained desired product were combined and concentrated. The residue was purified by Prep-HPLC with the following conditions Column: Xselect CSH OBD Column 30*150 mm 5 um, n; Mobile Phase A: Water (10 mmol/L NH₄HCO₃), Mobile Phase B: ACN; Flow rate: 30 mL/min; Gradient: 20 B to 60 B in 8 min; 210/254 nm; RT1: 7.78. The fractions contained desired product were combined and concentrated to afford 3-[2-(2-ethylindazol-5-yl)ethynyl]-1-[(3S,5R)-5-(methoxymethyl)-1-(prop-2-enoyl)pyrrolidin-3-yl]-5-(methylamino)pyrazole-4-carboxamide (71.4 mg, 23%) as an off-white solid. MS ESI calculated for C₂₅H₂₉N₇O₃ [M+H]⁺, 476.23, found 476.20. H-NMR (400 MHz, DMSO-d₆): 8.50 (s, 1H), 8.03 (s, 1H), 7.70-7.62 (m, 1H), 7.32 (dd, J=8.9, 1.6 Hz, 2H), 6.88-6.47 (m, 3H), 6.19-6.17 (m, 1H), 5.71-5.68 (m, 1H), 5.27-5.23 (m, 1H), 4.52-4.48 (m, 3H), 4.06-3.41 (m, 4H), 3.30 (d, J=5.4 Hz, 3H), 2.95 (t, J=5.3 Hz, 3H), 2.64-2.61 (m, 1H), 2.35-2.31 (m, 1H), 1.55-1.52 (m, 3H).

### Example 16: 1-((3S,5R)-1-acryloyl-5-(methoxymethyl)pyrrolidin-3-yl)-3-((1-ethyl-4,6-difluoro-2-methyl-1H-benzo[d]imidazol-5-yl)ethynyl)-5-(methylamino)-1H-pyrazole-4-carboxamide

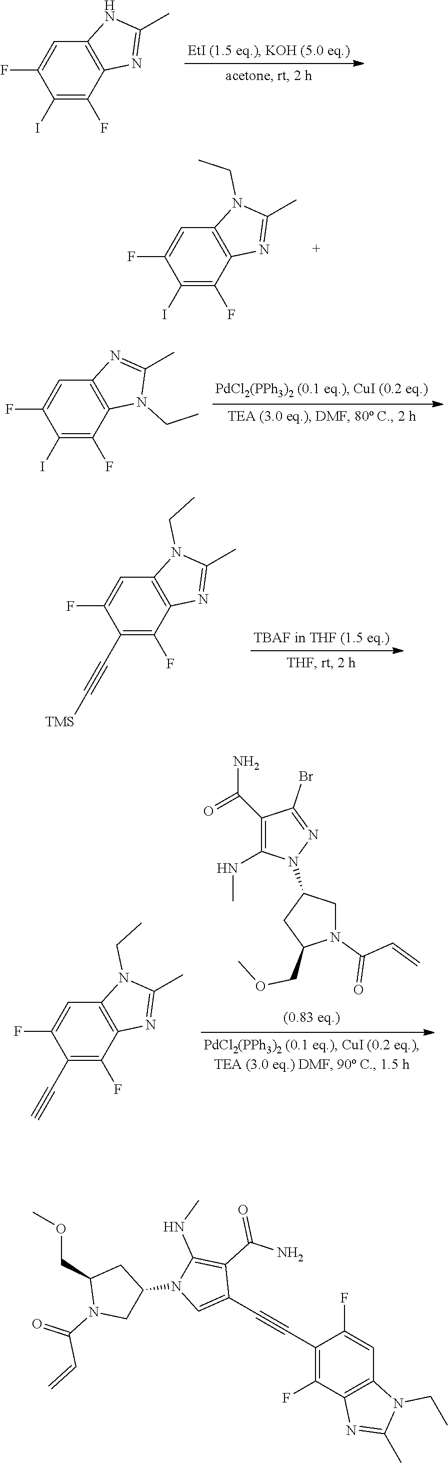

### Step 1: 1-ethyl-4,6-difluoro-5-iodo-2-methyl-1,3-benzodiazole

To a stirred solution of 4,6-difluoro-5-iodo-2-methyl-1H-1, 3-benzodiazole (3.00 g, 10.20 mmol) and KOH (2.86 g, 51.01 mmol) in THF (45.00 mL) was added ethyl iodide (2.39 g, 15.30 mmol) dropwise at room temperature. The resulting mixture was stirred for 2 h at room temperature. The resulting mixture was extracted with EtOAc (3×500 mL). The combined organic layers were washed with brine (3×50 mL), dried over anhydrous Na₂SO₄ and filtered. The filtrate was concentrated under reduced pressure. The residue was purified by silica gel column chromatography, eluted with CH₂Cl₂/EtOAc (50-89%). The fractions contained desired product were combined and concentrated to afford 1-ethyl-4,6-difluoro-5-iodo-2-methyl-1,3-benzodiazole (1.20 g, 36%) as a yellow solid and 1-ethyl-5,7-difluoro-6-iodo-2-methyl-1,3-benzodiazole (1.00 g, 30%) as a yellow solid. MS ESI calculated for C₁₀H₉F₂IN₂ [M+H]⁺, 322.97, found 323.09.

### Step 2: 1-ethyl-4,6-difluoro-2-methyl-5-[2-(trimethylsilyl)ethynyl]-1,3-benzodiazole

Into a 50 mL vial were added 1-ethyl-4,6-difluoro-5-iodo-2-methyl-1,3-benzodiazole (1.23 g, 3.82 mmol), trimethylsilylacetylene (1.13 g, 11.46 mmol), Pd(PPh₃)₂Cl₂ (0.27 g, 0.38 mmol), CuI (0.15 g, 0.76 mmol) and TEA (1.16 g, 11.46 mmol) in DMF (10.00 mL) at room temperature. The reaction mixture was degassed with argon for three times and stirred for 2 h at 80° C. The resulting mixture was allowed to cool down to room temperature. The resulting mixture was concentrated under reduced pressure. The residue was purified by silica gel column chromatography, eluted with PE/EtOAc (0-75%). The fractions contained desired product were combined and concentrated to afford 1-ethyl-4,6-difluoro-2-methyl-5-[2-(trimethylsilyl)ethynyl]-1,3-benzodiazole (1.1 g, 98%) as a yellow solid. MS ESI calculated for C₁₅H₁₈F₂N₂Si [M+H]⁺, 293.12, found 293.40.

### Step 3: 1-ethyl-5-ethynyl-4,6-difluoro-2-methyl-1,3-benzodiazole

To a stirred solution of 1-ethyl-4,6-difluoro-2-methyl-5-[2-(trimethylsilyl)ethynyl]-1,3-benzodiazole (1.20 g 4.10 mmol) in THF (10.00 mL) was added TBAF (1.61 g, 6.16 mmol) in portions at room temperature. The reaction mixture was stirred for 2 h at room temperature. The resulting mixture was concentrated under reduced pressure. The residue was diluted with water (150 mL), extracted with EtOAc (3×150 mL). The combined organic layers were washed with water (3×100 mL), dried over anhydrous Na₂SO₄ and filtered. The filtrate was concentrated under reduced pressure. The residue was purified by silica gel column chromatography, eluted with PE/EtOAc (20-60%). The fractions contained desired product were combined and concentrated to afford 1-ethyl-5-ethynyl-4, 6-difluoro-2-methyl-1,3-benzodiazole (0.67 g, 74%) as a yellow solid. MS ESI calculated for C₁₅H₁₈F₂N₂Si [M+H]⁺, 221.08, found 221.22.

### Step 4: 3-[2-(1-ethyl-4,6-difluoro-2-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

Into a 25 mL vial were added 3-bromo-1-[(3S,5R)-5-(methoxymethyl)-1-(prop-2-enoyl)pyrrolidin-3-yl]-5-(methylamino)pyrazole-4-carboxamide (0.20 g, 0.52 mmol), 1-ethyl-5-ethynyl-4,6-difluoro-2-methyl-1,3-benzodiazole (0.14 g, 0.62 mmol), Pd(PPh₃)₂Cl₂ (36.34 mg, 0.05 mmol) and CuI (19.72 mg, 0.10 mmol) in DMF (4.00 mL) was added TEA (0.16 g, 1.55 mmol) at room temperature. The reaction mixture was degassed with argon for three times and stirred for 1.5 h at 90° C. The mixture was allowed to cool down to room temperature. The resulting mixture was extracted with EtOAc (3×100 mL). The combined organic layers were washed with water (3×50 mL), dried over anhydrous Na₂SO₄ and filtered. The filtrate was concentrated under reduced pressure. The residue was purified by silica gel column chromatography, eluted with CH₂Cl₂/MeOH (0-6%). The fractions contained desired product were combined and concentrated. The crude product (160 mg) was purified by Prep-HPLC with the following conditions Column: XBridge Prep C18 OBD Column, 19×150 mm 5 um; Mobile Phase A: Water (10 mmol/L NH₄HCO₃), Mobile Phase B: ACN; Flow rate: 20 mL/min; Gradient: 23 B to 43 B in 4.3 min; 210/254 nm; RT1: 4.02. The fractions contained desired product were combined and concentrated to afford 3-[2-(1-ethyl-4,6-difluoro-2-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (85.7 mg, 31%) as a white solid. MS ESI calculated for C₂₆H₂₉F₂N₇O₃[M+H]⁺, 512.21, found 526.56. 1H-NMR (400 MHz, DMSO-d₆) δ 7.60 (t, J=11.4 Hz, 2H), 6.83-6.50 (m, 3H), 6.19-6.16 (m, 1H), 5.71-5.68 (m, 1H), 5.29-5.25 (m, 1H), 4.46 (d, J=54.4 Hz, 1H), 4.24 (q, J=7.2 Hz, 2H), 4.07-3.71 (m, 2H), 3.66-3.41 (m, 2H), 3.30 (d, J=5.6 Hz, 3H), 2.96 (t, J=5.5 Hz, 3H), 2.70-2.60 (m, 1H), 2.57 (s, 3H), 2.37-2.25 (m, 1H), 1.29 (t, J=7.2 Hz, 3H).

### Example 17: 1-((3S,5R)-1-acryloyl-5-(methoxymethyl)pyrrolidin-3-yl)-3-((4,6-difluoro-2-methyl-1H-benzo[d]imidazol-5-yl)ethynyl)-5-(methylamino)-1H-pyrazole-4-carboxamide

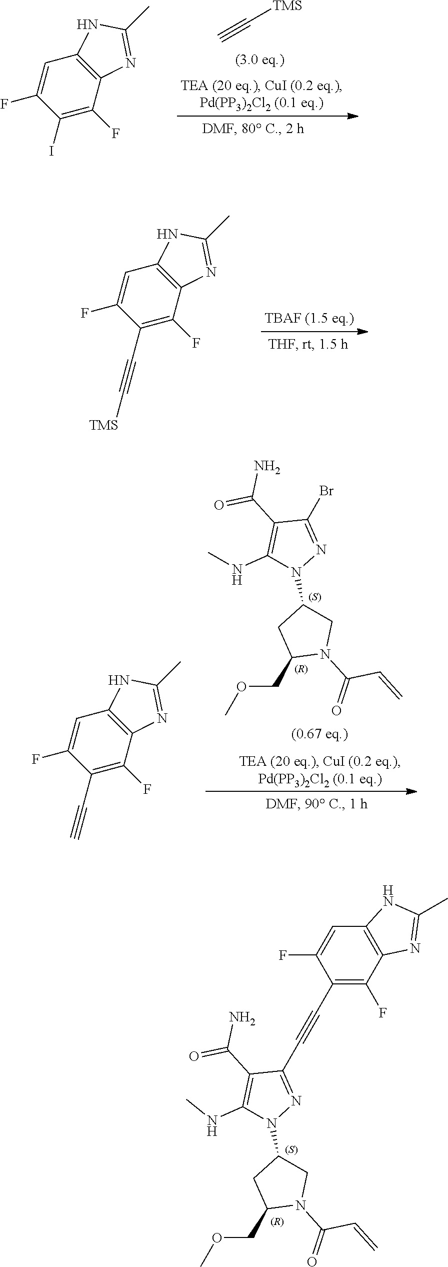

### Step 1: 4,6-difluoro-2-methyl-5-[2-(trimethylsilyl)ethynyl]-1H-1,3-benzodiazole

To a stirred mixture of 4,6-difluoro-5-iodo-2-methyl-1H-1,3-benzodiazole (0.60 g, 2.04 mmol), trimethylsilylacetylene (0.60 g, 6.12 mmol), Pd(PPh₃)₂Cl₂ (0.14 g, 0.20 mmol) and CuI (77.72 mg, 0.41 mmol) in DMF (10.00 mL) was added TEA (4.13 g, 40.81 mmol). The reaction mixture was degassed with nitrogen for three times and stirred for 2 h at 80° C. The resulting mixture was diluted with water (50 mL). The resulting mixture was extracted with EtOAc (3×50 mL). The combined organic layers was dried over anhydrous Na₂SO₄ and filtered. The filtrate was concentrated under reduced pressure. The residue was purified by silica gel column chromatography, eluted with PE/EtOAc (1:4). The fractions contained desired product were combined and concentrated to afford 4,6-difluoro-2-methyl-5-[2-(trimethylsilyl)ethynyl]-1H-1,3-benzodiazole (0.40 g, 74%) as a brown semi-solid. MS ESI calculated for C₁₃H₁₄F₂N₂Si [M+H]⁺, 265.09, found 265.10.

### Step 2: 5-ethynyl-4,6-difluoro-2-methyl-1H-1,3-benzodiazole

Into a 25 mL round-bottom flask were added 4,6-difluoro-2-methyl-5-[2-(trimethylsilyl)ethynyl]-1H-1,3-benzodiazole (0.40 g, 1.51 mmol), TBAF (2.27 mL, 2.27 mmol) and THF (4.00 mL) at 0° C. The reaction mixture was stirred for 1.5 h at room temperature. The resulting mixture was concentrated under reduced pressure. The residue was purified by silica gel column chromatography, eluted with CH₂Cl₂/MeOH (10:1). The fractions contained desired product were combined and concentrated to afford 5-ethynyl-4,6-difluoro-2-methyl-1H-1,3-benzodiazole (0.22 g, 75%) as a light yellow solid. MS ESI calculated for C₁₀H₆F₂N₂ [M+H]⁺, 193.05, found 193.15.

### Step 3: 3-[2-(4,6-difluoro-2-methyl-1H-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a mixture of 3-bromo-1-[(3S,5R)-5-(methoxymethyl)-1-(prop-2-enoyl)pyrrolidin-3-yl]-5-(methylamino)pyrazole-4-carboxamide (0.20 g, 0.52 mmol) and 5-ethynyl-4,6-difluoro-2-methyl-1H-1,3-benzodiazole (0.15 g, 0.78 mmol) in DMF (4.00 mL) were added CuI (19.72 mg, 0.10 mmol), Pd(PPh₃)₂Cl₂ (36.34 mg, 0.05 mmol) and TEA (1.05 g, 10.34 mmol). The reaction mixture was degassed with argon for three times and stirred for 1 h at 90° C. The resulting mixture was filtered, the filter cake was washed with MeOH (3×10 mL). The filtrate was concentrated under reduced pressure. The residue was purified by reverse flash chromatography with the following conditions: column, C18 silica gel; mobile phase, ACN in water (10 mM NH₄HCO₃), 5% to 35% gradient in 30 min; detector, UV 220 nm. The crude product (150 mg) was purified by Prep-HPLC with the following conditions Column: SunFire Prep C18 OBD Column, 19×150 mm 5 μm 10 nm; Mobile Phase A: Water (0.1% FA), Mobile Phase B: ACN; Flow rate: 20 mL/min; Gradient: 40 B to 80 B in 4.3 min; 210/254 nm; RT1:4.03. The fractions contained desired product were combined and concentrated to afford 3-[2-(4,6-difluoro-2-methyl-1H-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (43.3 mg, 16%) as a white solid. MS ESI calculated for C₂₄H₂₅F₂N₇O₃[M+H]⁺, 498.20, found 498.20. H-NMR (400 MHz, DMSO-d₆): δ 7.59 (s, 1H), 7.37 (d, J=9.1 Hz, 1H), 6.81-6.67 (m, 2H), 6.63-6.60 (m, 1H), 6.18-6.16 (m, 1H), 5.69-5.65 (m, 1H), 5.35-5.21 (m, 1H), 4.53-4.51 (m, 1H), 4.41-4.39 (m, 1H), 4.05-4.01 (m, 1H), 3.90-3.87 (m, 1H), 3.77-3.75 (m, 1H), 3.63-3.59 (m, 1H), 3.49-3.45 (m, 3H), 3.30 (d, J=5.6 Hz, 3H), 2.96 (t, J=5.5 Hz, 3H), 2.69-2.65 (m, 1H), 2.36-2.23 (m, 1H).

### Example 18: 1-((3S,5R)-1-acryloyl-5-(methoxymethyl)pyrrolidin-3-yl)-3-((4,6-difluoro-1,2-dimethyl-1H-benzo[d]imidazol-5-yl)ethynyl)-5-(methylamino)-1H-pyrazole-4-carboxamide

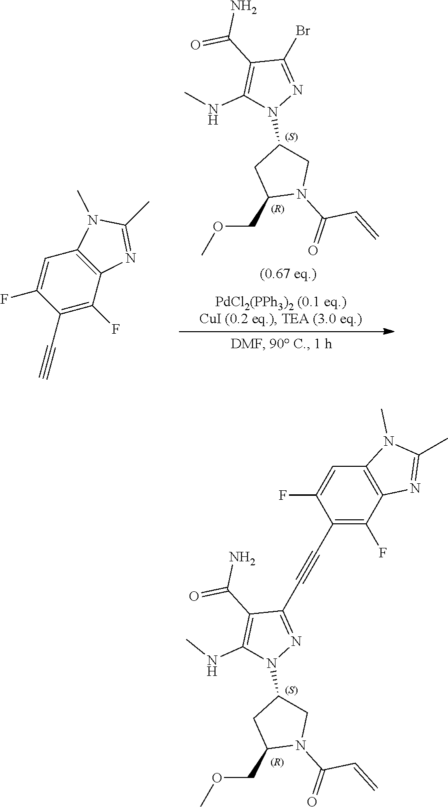

To a stirred mixture of 3-bromo-1-[(3S,5R)-5-(methoxymethyl)-1-(prop-2-enoyl)pyrrolidin-3-yl]-5-(methylamino)pyrazole-4-carboxamide (0.30 g, 0.78 mmol) in DMF (5.00 mL) were added 5-ethynyl-4,6-difluoro-1,2-dimethyl-1,3-benzodiazole (0.19 g, 0.93 mmol), Pd(PPh₃)₂Cl₂ (54.52 mg, 0.08 mmol), CuI (29.58 mg, 0.16 mmol) and TEA (0.24 g, 2.33 mmol) at room temperature. The reaction mixture was degassed with argon for three times and stirred for 1 h at 90° C. The mixture was allowed to cool down to room temperature. The resulting mixture was filtered; the filter cake was washed with DMF (3×3 mL). The residue was purified by reverse phase flash chromatography with the following conditions: Column: Spherical C18, 20-40 um, 180 g; Mobile Phase A: Water (NH₄HCO₃ 0.1 mM); Mobile Phase B: ACN; Flow rate: 90 mL/min; Gradient: 5%-5% B, 10 min, 25% B-60% B gradient in 20 min; Detector: 254 nm. The fractions containing the desired product were collected at 30% B and concentrated under reduced pressure to afford 3-[2-(4, 6-difluoro-1, 2-dimethyl-1, 3-benzodiazol-5-yl) ethynyl]-1-[(3S,5R)-5-(methoxymethyl)-1-(prop-2-enoyl) pyrrolidin-3-yl]-5-(methylamino) pyrazole-4-carboxamide as a white solid (83.7 mg, 21%) as a white solid. MS ESI calculated for C₂₅H₂₇F₂N₇O₃[M+H]⁺, 512.21, found 512.53. 1H-NMR (300 MHz, DMSO-4) S 7.58 (d, J=9.3 Hz, 2H), 6.88-6.51 (m, 3H), 6.23-6.11 (m, 1H), 5.73-5.69 (m, 1H), 5.29-5.26 (m, 1H), 4.48 (d, J=41.7 Hz, 1H), 4.10-3.83 (m, 2H), 3.76 (s, 3H), 3.68-3.43 (m, 2H), 3.35-3.32 (m, 3H), 3.00-2.95 (m, 3H), 2.69-2.63 (m, 1H), 2.56 (s, 3H), 2.39-2.24 (m, 1H).

### Example 19: 1-((3S,5R)-1-acryloyl-5-(methoxymethyl)pyrrolidin-3-yl)-3-((4,6-difluoro-1-methyl-1H-indazol-5-yl)ethynyl)-5-(methylamino)-1H-pyrazole-4-carboxamide

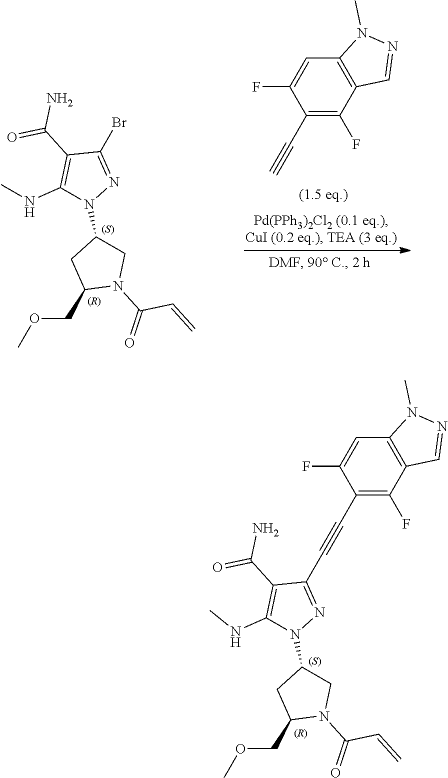

To a stirred mixture of 3-bromo-1-[(3S,5R)-5-(methoxymethyl)-1-(prop-2-enoyl)pyrrolidin-3-yl]-5-(methylamino)pyrazole-4-carboxamide (0.15 g, 0.39 mmol), 5-ethynyl-4,6-difluoro-1-methylindazole (0.11 g, 0.58 mmol), Pd(PPh₃)₂Cl₂ (27.26 mg, 0.04 mmol) and CuI (14.79 mg, 0.08 mmol) in DMF (2.00 mL) was added TEA (0.12 g, 1.17 mmol). The reaction mixture was degassed with argon for three times and stirred for 2 h at 90° C. The resulting mixture was concentrated under reduced pressure. The residue was purified by reverse flash chromatography with the following conditions: column, C18 silica gel; mobile phase ACN, NH₄HCO₃ 0.01 mmol in water, 25% to 40% gradient in 20 min; detector, UV 254 nm. The crude product was purified by Prep-HPLC with the following conditions Column: Atlantis Prep T3 OBD Column, 19*250 mm 10 u; Mobile Phase A: Water (0.1% FA), Mobile Phase B: ACN; Flow rate: 20 mL/min; Gradient: 25 B to 50 B in 6 min; 210/254 nm; RT1:5.56. The fractions contained desired product were combined and concentrated to afford 3-[2-(4,6-difluoro-1-methylindazol-5-yl)ethynyl]-1-[(3S,5R)-5-(methoxymethyl)-1-(prop-2-enoyl)pyrrolidin-3-yl]-5-(methylamino)pyrazole-4-carboxamide (27.5 mg, 14%) as a white solid. ESI calculated for C₂₄H₂₅F₂N₇O₃[M+H]⁺, 498.2; found 498.2. ¹H NMR (400 MHz, DMSO-d₆) δ 8.33 (s, 1H), 7.72 (d, J=9.5 Hz, 1H), 7.57 (s, 1H), 6.91-6.53 (m, 3H), 6.17-5.92 (m, 1H), 5.69-5.45 (m, 1H), 5.41-5.18 (m, 1H), 4.54-4.34 (m, 1H), 4.06 (s, 3H), 3.88-3.65 (m, 1H), 3.63-3.59 (m, 1H), 3.55-3.39 (m, 2H), 3.31 (s, 3H), 2.98-2.94 (m, 3H), 2.62-2.43 (m, 1H), 2.30-1.96 (m, 1H).

### Example 20: 1-((3S,5R)-1-acryloyl-5-(methoxymethyl)pyrrolidin-3-yl)-3-((4-fluoro-1,2-dimethyl-1H-benzo[d]imidazol-5-yl)ethynyl)-5-(methylamino)-1H-pyrazole-4-carboxamide

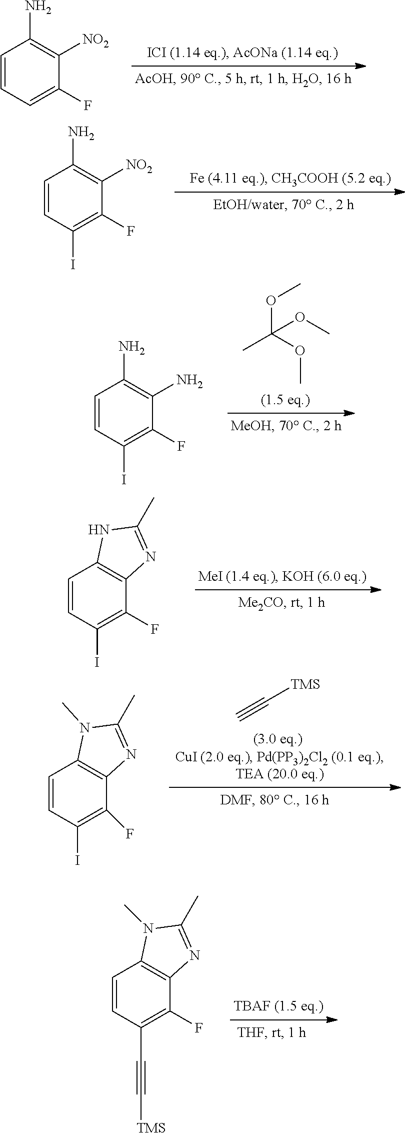

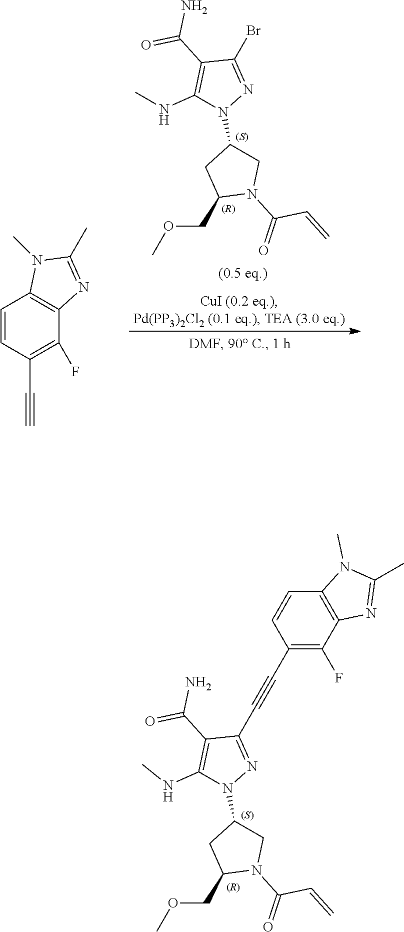

### Step 1: 3-fluoro-4-iodo-2-nitroaniline

To a stirred solution of 3-fluoro-2-nitroaniline (14.50 g, 92.88 mmol) and NaOAc (8.69 g, 105.93 mmol) in AcOH (250.00 mL) was added ICl (17.19 g, 105.88 mmol) in AcOH (50.00 mL) dropwise at room temperature under nitrogen atmosphere. The reaction mixture was stirred for additional 5 h at 90° C., then stirred for 1 h at room temperature. To the above mixture was added water (300.00 mL) at room temperature, and was stirred for 16 h. The resulting mixture was filtered, the filter cake was washed with water (3×80 mL). The residue was purified by silica gel column chromatography, eluted with PE/EtOAc (4:1), the fractions contained desired product were combined and concentrated to afford 3-fluoro-4-iodo-2-nitroaniline (13.1 g, 50%) as an orange solid.

### Step 2: 3-fluoro-4-iodobenzene-1,2-diamine

A mixture of Fe (10.66 g, 190.92 mmol) and CH₃COOH (13.84 mL, 241.53 mmol) in EtOH (150.00 mL) and water (50.00 mL) was stirred for 0.5 h at 70° C. To the above mixture was added 3-fluoro-4-iodo-2-nitroaniline (13.10 g, 46.45 mmol) in portions at 70° C. The resulting mixture was stirred for additional 2 h at 70° C. The mixture was cooled. The resulting mixture was filtered, the filter cake was washed with EA (2×150 mL). The filtrate was concentrated under reduced pressure. The residue was added water (70 mL) and extracted with EA (3×100 mL). The combined organic layers were washed with Sat. NaCl (2×70 mL), dried over anhydrous Na₂SO₄ and filtered. The residue was concentrated to afford 3-fluoro-4-iodobenzene-1,2-diamine (11.2 g, 95.7%) as a brown solid which was used in the next step without further purification.

### Step 3: 4-fluoro-5-iodo-2-methyl-1H-benzo[d]imidazole

To a stirred solution of 3-fluoro-4-iodobenzene-1,2-diamine (5.00 g 19.84 mmol) in MeOH (50.00 mL) was added 1,1,1-trimethoxyethane (3.58 g, 29.79 mmol) dropwise at room temperature under nitrogen atmosphere. The reaction mixture was stirred for 2 h at 70° C. under nitrogen atmosphere. The resulting mixture was concentrated under reduced pressure. The residue was purified by silica gel column chromatography, eluted with PE/EtOAc (1:4), the fractions contained desired product were combined and concentrated to afford 4-fluoro-5-iodo-2-methyl-1H-benzo[d]imidazole (5.15 g, 94%) as a light yellow solid. MS ESI calculated for C₈H₆FIN₂ [M+H]⁺, 276.96, found 276.95.

### Step 4: 4-fluoro-5-iodo-1,2-dimethyl-1H-benzo[d]imidazole

To a stirred solution of 4-fluoro-5-iodo-2-methyl-1H-benzo[d]imidazole (2.35 g, 8.51 mmol) and KOH (2.87 g, 51.15 mmol) in acetone (25.00 mL) was added MeI (0.74 mL, 5.23 mmol) dropwise at room temperature under nitrogen atmosphere. The reaction mixture was stirred for 40 min. The resulting mixture was extracted with EtOAc (3×60 mL). The combined organic layers were washed with brine (3×50 mL), dried over anhydrous Na₂SO₄ and filtered. The filtrate was concentrated under reduced pressure. The residue was purified by silica gel column chromatography, eluted with CH₂Cl₂/MeOH (10:1), the fractions contained desired product were combined and concentrated to afford 4-fluoro-5-iodo-1,2-dimethyl-1H-benzo[d]imidazole (1.13 g, 45%) as an off-white solid. MS ESI calculated for C₉H₈FIN₂ [M+H]⁺, 290.97, found 290.95.

### Step 5: 4-fluoro-1,2-dimethyl-5-((trimethylsilyl)ethynyl)-1H-benzo[d]imidazole

To a mixture of 4-fluoro-5-iodo-1,2-dimethyl-1H-benzo[d]imidazole (1.13 g, 3.89 mmol), trimethylsilylacetylene (1.15 g, 11.71 mmol), CuI (0.15 g, 0.78 mmol) and Pd(PPh₃)₂Cl₂ (0.27 g, 0.39 mmol) in DMF (12.00 mL) was added TEA (10.83 mL, 77.92 mmol). The reaction mixture was degassed with argon for three times and stirred for 16 h at 80° C. The resulting mixture was extracted with EtOAc (3×80 mL). The combined organic layers were washed with water (3×60 mL), dried over anhydrous Na₂SO₄ and filtered. The filtrate was concentrated under reduced pressure. The residue was purified by silica gel column chromatography, eluted with PE/EtOAc (1:2), the fractions contained desired product were combined and concentrated to afford 4-fluoro-1,2-dimethyl-5-((trimethylsilyl)ethynyl)-1H-benzo[d]imidazole (0.94 g, 92%) as a light yellow solid. MS ESI calculated for C₁₄H₁₇FN₂Si [M+H]⁺, 261.11, found 261.15.

### Step 6: 5-ethynyl-4-fluoro-1,2-dimethyl-1H-benzo[d]imidazole

To a stirred solution of 4-fluoro-1,2-dimethyl-5-((trimethylsilyl)ethynyl)-1H-benzo[d]imidazole (0.84 g, 3.23 mmol) in THF (10.00 mL) was added TBAF (4.84 mL, 4.84 mmol) dropwise at 0° C. under nitrogen atmosphere. The reaction mixture was stirred for 1 h at room temperature. The resulting mixture was concentrated under reduced pressure. The residue was purified by silica gel column chromatography, eluted with PE/EtOAc (1:2), the fractions contained desired product were combined and concentrated to afford 5-ethynyl-4-fluoro-1,2-dimethyl-1H-benzo[d]imidazole (0.67 g, 99%) as an off-white solid. MS ESI calculated for C₁₁H₉FN₂ [M+H]⁺, 189.07, found 189.20.

### Step 7: 1-((3S,5R)-1-acryloyl-5-(methoxymethyl)pyrrolidin-3-yl)-3-((4-fluoro-1,2-dimethyl-1H-benzo[d]imidazol-5-yl)ethynyl)-5-(methylamino)-1H-pyrazole-4-carboxamide

To a mixture of 3-bromo-1-[(3S,5R)-5-(methoxymethyl)-1-(prop-2-enoyl)pyrrolidin-3-yl]-5-(methylamino)pyrazole-4-carboxamide (0.2 g, 0.52 mmol), 5-ethynyl-4-fluoro-1,2-dimethyl-1H-benzo[d]imidazole (0.19 g, 1.04 mmol), CuI (19.72 mg, 0.10 mmol) and Pd(PPh₃)₂Cl₂ (36.34 mg, 0.05 mmol) in DMF (2.00 mL) was added TEA (0.22 mL, 1.58 mmol). The reaction mixture was degassed with argon for three times and stirred for 1 h at 90° C. The residue was purified by Prep-HPLC with the following conditions: Column: XBridge Prep OBD C18 Column, 19*250 mm, 5 um; Mobile Phase A: Water (10 mmol/L NH₄HCO₃), Mobile Phase B: ACN; Flow rate: 20 mL/min; Gradient: 25 B to 60 B in 5.8 min; 210/254 nm; RT1: 5.55, the fractions contained desired product were combined and concentrated to afford 1-((3S,5R)-1-acryloyl-5-(methoxymethyl)pyrrolidin-3-yl)-3-((4-fluoro-1,2-dimethyl-1H-benzo[d]imidazol-5-yl)ethynyl)-5-(methylamino)-1H-pyrazole-4-carboxamide (0.10 g, 39%) as an off-white solid. MS ESI calculated for C₂₅H₂₈FN₇O₃[M+H]⁺, 494.22, found 494.20. H NMR (400 MHz, DMSO-d₆) δ 7.43-7.38 (m, 3H), 6.76-6.56 (m, 3H), 6.19-6.14 (m, 1H), 5.70-5.67 (m, 1H), 5.28-5.26 (m, 1H), 4.53-4.39 (m, 1H), 3.79-3.75 (m, 5H), 3.62-3.44 (m, 2H), 3.32 (s, 3H), 2.97-2.94 (t, J=5.2 Hz, 3H), 2.58-2.52 (m, 4H), 2.32-2.27 (m, 1H).

### Example 21: 1-((3S,5R)-1-acryloyl-5-(methoxymethyl)pyrrolidin-3-yl)-3-((1-ethyl-4-fluoro-2-methyl-1H-benzo[d]imidazol-5-yl)ethynyl)-5-(methylamino)-1H-pyrazole-4-carboxamide

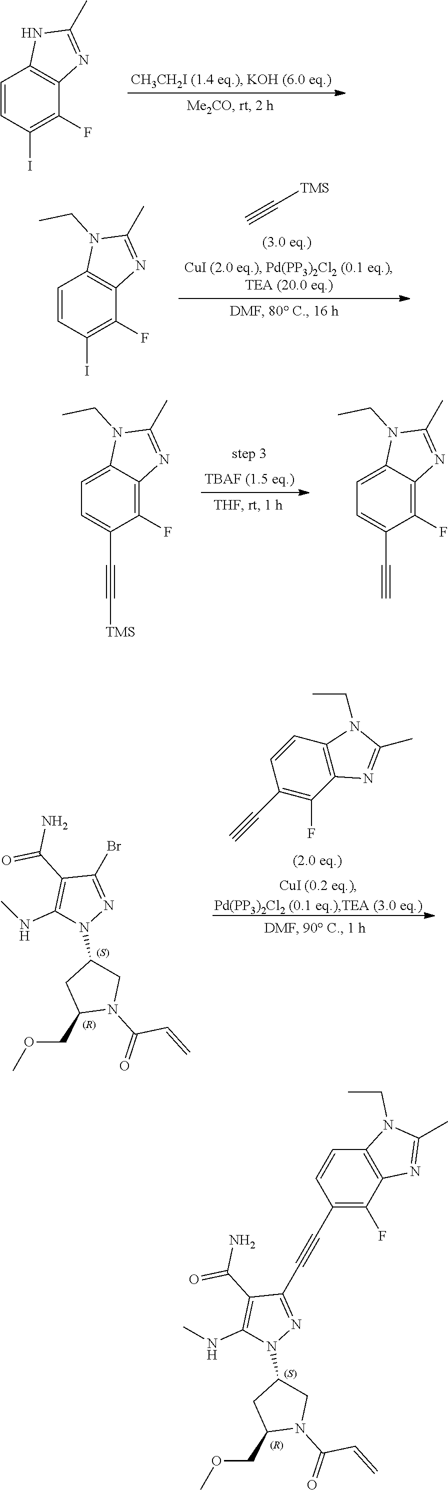

### Step 1: 1-ethyl-4-fluoro-5-iodo-2-methyl-1H-benzo[d]imidazole

To a stirred solution of 4-fluoro-5-iodo-2-methyl-1H-1,3-benzodiazole (2.35 g, 8.51 mmol) and KOH (2.87 g, 51.08 mmol) in acetone (25.00 mL) was added ethyl iodide (1.86 g, 11.93 mmol) dropwise at room temperature under nitrogen atmosphere. The resulting mixture was stirred for 2 h at room temperature under nitrogen atmosphere. The resulting mixture was extracted with EtOAc (3×60 mL). The combined organic layers were washed with brine (3×50 mL), dried over anhydrous Na₂SO₄ and filtered. The filtrate was concentrated under reduced pressure. The residue was purified by silica gel column chromatography, eluted with CH₂Cl₂/EtOAc (1:1), the fractions contained desired product were combined and concentrated to afford 1-ethyl-4-fluoro-5-iodo-2-methyl-1H-benzo[d]imidazole (1.46 g, 56.4%) as an off-white solid. MS ESI calculated for C₁₀H₁₀FIN₂ [M+H]⁺, 304.99, found 304.95.

### Step 2: 1-ethyl-4-fluoro-2-methyl-5-((trimethylsilyl)ethynyl)-1H-benzo[d]imidazole

To a mixture of 1-ethyl-4-fluoro-5-iodo-2-methyl-1H-benzo[d]imidazole (1.46 g, 4.80 mmol), trimethylsilylacetylene (1.41 g, 14.36 mmol), CuI (0.18 g, 0.96 mmol) and Pd(PPh₃)₂Cl₂ (0.34 g, 0.48 mmol) in DMF (15.00 mL) was added TEA (13.35 mL, 96.04 mmol). The reaction mixture was degassed with argon for three times and stirred for 16 h at 80° C. The resulting mixture was extracted with EtOAc (3×100 mL). The combined organic layers were washed with water (3×60 mL), dried over anhydrous Na₂SO₄ and filtered. The filtrate was concentrated under reduced pressure. The residue was purified by silica gel column chromatography, eluted with PE/EtOAc (1:2), the fractions contained desired product were combined and concentrated to afford 1-ethyl-4-fluoro-2-methyl-5-((trimethylsilyl)ethynyl)-1H-benzo[d]imidazole (1.0 g, 75%) as a light yellow solid. MS ESI calculated for C₁₅H₁₉FN₂Si [M+H]⁺, 275.13, found 275.15.

### Step 3: 1-ethyl-5-ethynyl-4-fluoro-2-methyl-1H-benzo[d]imidazole

To a stirred solution of 1-ethyl-4-fluoro-2-methyl-5-((trimethylsilyl)ethynyl)-1H-benzo[d]imidazole (0.91 g, 3.32 mmol) in THF (10.00 mL) was added TBAF (4.97 mL, 4.97 mmol) dropwise at 0° C. under nitrogen atmosphere. The resulting mixture was stirred for 1 h at room temperature under nitrogen atmosphere. The resulting mixture was concentrated under reduced pressure. The residue was purified by silica gel column chromatography, eluted with PE/EtOAc (1:2), the fractions contained desired product were combined and concentrated to afford 1-ethyl-5-ethynyl-4-fluoro-2-methyl-1H-benzo[d]imidazole (0.68 g, 96%) as an off-white solid. MS ESI calculated for C₁₂H₁₁FN₂ [M+H]⁺, 203.09, found 203.20.

### Step 4: 1-((3S,5R)-1-acryloyl-5-(methoxymethyl)pyrrolidin-3-yl)-3-((1-ethyl-4-fluoro-2-methyl-1H-benzo[d]imidazol-5-yl)ethynyl)-5-(methylamino)-1H-pyrazole-4-carboxamide

To a mixture of 3-bromo-1-[(3S,5R)-5-(methoxymethyl)-1-(prop-2-enoyl)pyrrolidin-3-yl]-5-(methylamino)pyrazole-4-carboxamide (0.2 g, 0.52 mmol), 1-ethyl-5-ethynyl-4-fluoro-2-methyl-1H-benzo[d]imidazole (0.21 g, 1.04 mmol), CuI (19.72 mg, 0.10 mmol) and Pd(PPh₃)₂Cl₂ (36.34 mg, 0.05 mmol) in DMF (2.00 mL) was added TEA (0.22 mL, 2.13 mmol). The reaction mixture was degassed with argon for three times and stirred for 1 h at 90° C. The resulting mixture was concentrated under reduced pressure. The residue was purified by Prep-HPLC with the following conditions: Column: XBridge Prep OBD C18 Column, 19*250 mm, 5 um; Mobile Phase A: Water (10 mmol/L NH₄HCO₃), Mobile Phase B: ACN; Flow rate: 20 mL/min; Gradient: 25 B to 60 B in 5.8 min; 210/254 nm; RT1: 5.56, the fractions contained desired product were combined and concentrated to afford 1-((3S,5R)-1-acryloyl-5-(methoxymethyl)pyrrolidin-3-yl)-3-((1-ethyl-4-fluoro-2-methyl-1H-benzo[d]imidazol-5-yl)ethynyl)-5-(methylamino)-1H-pyrazole-4-carboxamide (0.11 g, 41%) as an off-white solid. MS ESI calculated for C₂₆H₃₀FN₇O₃ [M+H]⁺, 508.24, found 508.25. H NMR (400 MHz, DMSO-d₆) δ 7.48-7.38 (m, 3H), 6.79-6.56 (m, 3H), 6.19-6.14 (m, 1H), 5.70-5.67 (m, 1H), 5.30-5.24 (m, 1H), 4.53-4.40 (m, 1H), 4.28-4.23 (m, 2H), 4.08-3.72 (m, 2H), 3.66-3.41 (m, 2H), 3.32-3.29 (m, 3H), 2.96 (t, J=5.2 Hz, 3H), 2.60-2.56 (m, 4H), 2.33-2.29 (m, 1H), 1.30 (t, J=7.1 Hz, 3H).

### Example 22: 1-((3S,5R)-1-acryloyl-5-(methoxymethyl)pyrrolidin-3-yl)-3-((3-ethyl-2-methyl-3H-imidazo[4,5-b]pyridin-6-yl)ethynyl)-5-(methylamino)-1H-pyrazole-4-carboxamide

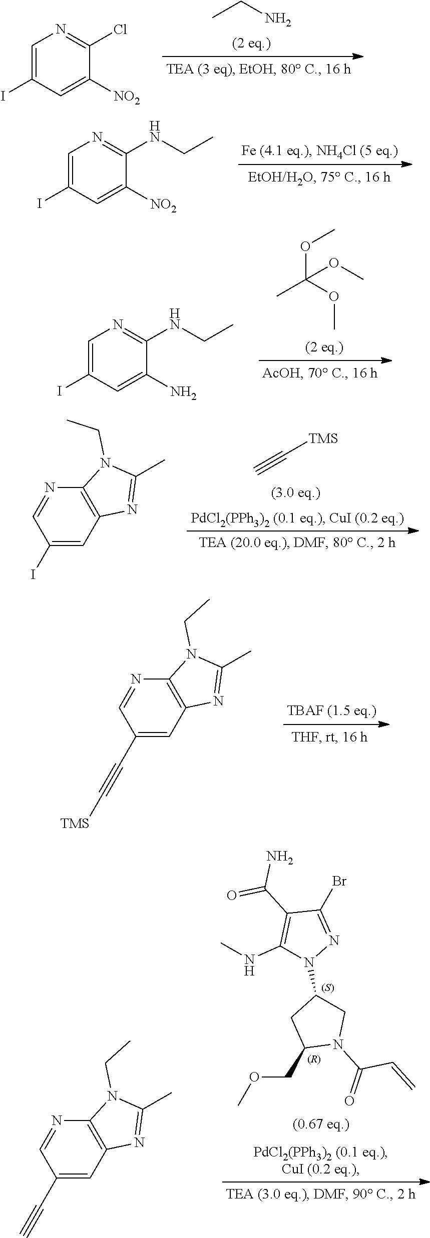

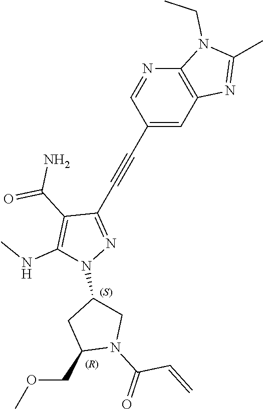

### Step 1: N-ethyl-5-iodo-3-nitropyridin-2-amine

To a stirred mixture of 2-chloro-5-iodo-3-nitropyridine (3.10 g, 10.90 mmol) and ethylamine (0.98 g, 21.8 mmol) in EtOH (30.00 mL) was added TEA (3.31 g, 32.69 mmol) at room temperature under air atmosphere. The reaction mixture was stirred for 16 h at 80° C. The resulting mixture was concentrated under reduced pressure. The residue was diluted with water (300 mL), extracted with EA (3×250 mL). The combined organic layers were washed with Sat. NaCl (500 mL), dried over anhydrous Na₂SO₄ and filtered. The residue was concentrated to afford N-ethyl-5-iodo-3-nitropyridin-2-amine (3.5 g, 99%) as an orang solid which was used in the next step without further purification. ESI calculated for C₇H₈IN₃O₂[M+H]⁺, 293.9; found 294.

### Step 2: N2-ethyl-5-iodopyridine-2,3-diamine

To a stirred mixture of N-ethyl-5-iodo-3-nitropyridin-2-amine (3.00 g, 10.24 mmol) and NH₄Cl (2.74 g, 51.18 mmol) in EtOH (52.00 mL) and water (8 mL) was added Fe (2.29 g, 40.95 mmol) at room temperature. The reaction mixture was stirred for 16 h at 75° C. under air atmosphere. The resulting mixture was filtered, the filter cake was washed with EA (2×150 mL). The filtrate was concentrated under reduced pressure. The residue was diluted with water (200 mL) and extracted with EA (3×200 mL). The combined organic layers were washed with Sat. NaCl (500 mL), dried over anhydrous Na₂SO₄ and filtered. The resulting mixture was concentrated to afford N2-ethyl-5-iodopyridine-2,3-diamine (2.6 g, 96%) as a black solid which was used in the next step without further purification. MS ESI calculated for C₇H₁₀IN₃ [M+H]⁺, 263.9; found 264.05.

### Step 3: 3-ethyl-6-iodo-2-methylimidazo[4,5-b]pyridine

To a stirred mixture of N₂-ethyl-5-iodopyridine-2,3-diamine (2.20 g, 8.36 mmol) in CH₃COOH (22.00 mL) was added 1,1,1-trimethoxyethane (2.01 g, 16.73 mmol) at room temperature under air atmosphere. The reaction mixture was stirred for 16 h at 70° C. The resulting mixture was concentrated under reduced pressure. The residue was diluted with EtOAc (100 mL). The mixture was basified to pH 8 with saturated NaHCO₃ (aq.). The resulting mixture was extracted with EtOAc (3×150 mL). The combined organic layers were washed with brine (200 mL), dried over anhydrous Na₂SO₄ and filtered. The filtrate was concentrated under reduced pressure. The residue was purified by silica gel column chromatography, eluted with CH₂Cl₂/MeOH (20:1). The fractions contained desired product were combined and concentrated to afford 3-ethyl-6-iodo-2-methylimidazo[4,5-b]pyridine (1.7 g, 70%) as a brown solid. MS ESI calculated for C₉H₁₀IN₃[M+H]⁺, 287.99; found 287.95.

### Step 4: 3-ethyl-2-methyl-6-[2-(trimethylsilyl)ethynyl]imidazo[4,5-b]pyridine

To a stirred mixture of 3-ethyl-6-iodo-2-methylimidazo[4,5-b]pyridine (1.50 g, 5.23 mmol), trimethylsilylacetylene (1.54 g, 15.67 mmol), Pd(PPh3)₂Cl₂ (0.37 g, 0.52 mmol) and CuI (0.2 g, 1.05 mmol) in DMF (14.00 mL) was added TEA (10.57 g, 104.49 mmol). The reaction mixture was degassed with argon for three times and stirred for 2 h at 80° C. The residue was diluted with water (150 mL), extracted with EtOAc (3×150 mL). The combined organic layers were washed with brine (200 mL), dried over anhydrous Na₂SO₄ and filtered. The filtrate was concentrated under reduced pressure. The residue was purified by silica gel column chromatography, eluted with CH₂Cl₂/MeOH (20:1). The fractions contained desired product were combined and concentrated to afford 3-ethyl-2-methyl-6-[2-(trimethylsilyl)ethynyl]imidazo[4,5-b]pyridine (1.1 g, 81%) as a brown solid. MS ESI calculated for C₁₄H₁₉N₃Si[M+H]⁺, 258.13; found 258.05.

### Step 5: 3-ethyl-6-ethynyl-2-methylimidazo[4,5-b]pyridine

To a stirred mixture of 3-ethyl-2-methyl-6-[2-(trimethylsilyl)ethynyl]imidazo[4,5-b]pyridine (1.00 g, 3.89 mmol) in THF (10.00 mL) was added TBAF (1.52 g, 5.83 mmol) at room temperature under air atmosphere. The reaction mixture was stirred for 16 h at room temperature. The resulting mixture was concentrated under reduced pressure. The residue was diluted with water (100 mL), extracted with EtOAc (3×150 mL). The combined organic layers were washed with brine (200 mL), dried over anhydrous Na₂SO₄ and filtered. The filtrate was concentrated under reduced pressure. The residue was purified by silica gel column chromatography, eluted with CH₂Cl₂/MeOH (10:1). The fractions contained desired product were combined and concentrated to afford 3-ethyl-6-ethynyl-2-methylimidazo[4,5-b]pyridine (0.28 g, 38%) as a brown solid. MS ESI calculated for C₁₁H₁₁N₃[M+H]⁺, 186.1; found 186.15.

### Step 6: 3-(2-[3-ethyl-2-methylimidazo[4,5-b]pyridin-6-yl]ethynyl)-1-[(3S,5R)-5-(methoxymethyl)-1-(prop-2-enoyl)pyrrolidin-3-yl]-5-(methylamino)pyrazole-4-carboxamide

To a stirred mixture of 3-bromo-1-[(3S,5R)-5-(methoxymethyl)-1-(prop-2-enoyl)pyrrolidin-3-yl]-5-(methylamino)pyrazole-4-carboxamide (0.2 g, 0.52 mmol), 3-ethyl-6-ethynyl-2-methylimidazo[4,5-b]pyridine (0.14 g, 0.78 mmol), Pd(PPh3)₂Cl₂ (36.34 mg, 0.05 mmol) and CuI (19.72 mg, 0.1 mmol) in DMF (2.00 mL) was added TEA (0.16 g, 1.55 mmol) dropwise at room temperature. The reaction mixture was degassed with argon for three times and stirred for 2 h at 90° C. The residue was purified by reverse flash chromatography with the following conditions: column, C18 silica gel; mobile phase ACN, NH₄HCO₃ 0.01 mmol in water, 20% to 40% gradient in 20 min; detector, UV 254 nm. The fractions contained desired product were combined and concentrated. The residue was purified by Prep-HPLC with the following conditions Column: XBridge Prep C18 OBD Column, 19×150 mm 5 um; Mobile Phase A: Water (10 mmol/L NH₄HCO₃), Mobile Phase B: ACN; Flow rate: 20 mL/min; Gradient: 25 B to 55 B in 6 min; 210/254 nm; RT1: 5.53. The fractions contained desired product were combined and concentrated to afford 3-(2-[3-ethyl-2-methylimidazo[4,5-b]pyridin-6-yl]ethynyl)-1-[(3S,5R)-5-(methoxymethyl)-1-(prop-2-enoyl)pyrrolidin-3-yl]-5-(methylamino)pyrazole-4-carboxamide (59 mg, 23%) as a light yellow solid. MS ESI calculated for C₂₅H₃₀N₈O₃ [M+H]⁺, 491.24; found 491.15. ¹H NMR (400 MHz, DMSO-d₆) δ 8.49 (d, J=1.9 Hz, 1H), 8.19-8.15 (m, 1H), 7.31 (s, 1H), 6.94-6.71 (m, 1H), 6.69-6.47 (m, 2H), 6.17-5.96 (m, 1H), 5.69-5.43 (m, 1H), 5.23-5.03 (m, 1H), 4.49-4.45 (m, 1H), 4.29 (q, J=7.3 Hz, 2H), 4.11-3.71 (m, 2H), 3.68-3.41 (m, 2H), 3.31 (d, J=5.4 Hz, 3H), 3.06-2.89 (m, 3H), 2.65-2.60 (m, 4H), 2.38-2.25 (m, 1H), 1.34 (t, J=7.2 Hz, 3H).

### Example 23: 1-((3S,5R)-1-acryloyl-5-(methoxymethyl)pyrrolidin-3-yl)-3-((3-methyl-3H-imidazo[4,5-b]pyridin-6-yl)ethynyl)-5-(methylamino)-1H-pyrazole-4-carboxamide

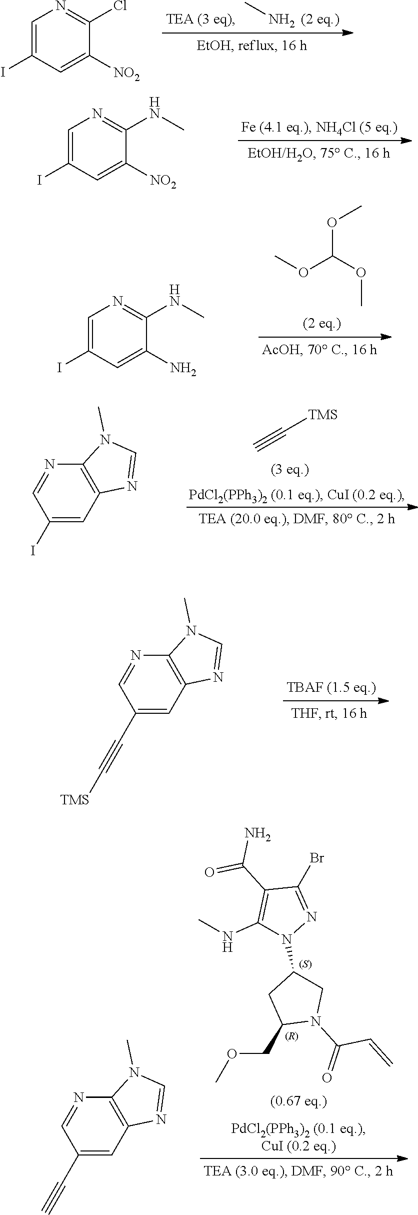

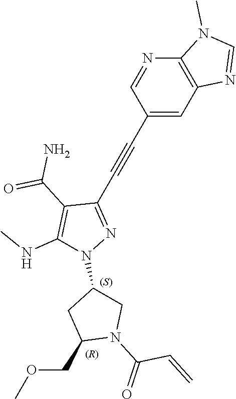

### Step 1: 5-iodo-N-methyl-3-nitropyridin-2-amine

To a stirred mixture of 2-chloro-5-iodo-3-nitropyridine (3.00 g, 10.55 mmol) and methylamine (0.65 g, 21.09 mmol) in EtOH (30.00 mL) was added TEA (3.20 g, 31.64 mmol) at room temperature under air atmosphere. The reaction mixture was stirred for 16 h at 80° C. The resulting mixture was concentrated under reduced pressure. The residue was diluted with water (300 mL), extracted with EA (3×150 mL). The combined organic layers were washed with Sat. NaCl (500 mL), dried over anhydrous Na₂SO₄ and filtered. The filtrate was concentrated and dried to afford 5-iodo-N-methyl-3-nitropyridin-2-amine (2.9 g, 98%) as an orang solid which was used in the next step directly without further purification. ESI calculated for C₆H₆IN₃O₂[M+H]⁺, 279.95; found 279.95.

### Step 2: 5-iodo-N2-methylpyridine-2,3-diamine

To a stirred mixture of 5-iodo-N-methyl-3-nitropyridin-2-amine (3.00 g, 10.75 mmol) and NH₄Cl (2.88 g, 53.75 mmol) in EtOH (52.00 mL)/H₂O (7.00 mL) was added Fe (2.40 g, 43.00 mmol) at room temperature under air atmosphere. The reaction mixture was stirred for 16 h at 75° C. The resulting mixture was filtered, the filter cake was washed with EA (2×150 mL). The filtrate was concentrated under reduced pressure. The residue was diluted with water (200 mL) and extracted with EA (3×200 mL). The combined organic layers were washed with Sat. NaCl (500 mL), dried over anhydrous Na₂SO₄ and filtered. The filtrate were combined and concentrated to afford 5-iodo-N₂-methylpyridine-2,3-diamine (1.7 g, 63%) as a black solid which was used in the next step directly without further purification. MS ESI calculated for C₆H₈IN₃ [M+H]⁺, 249.98; found 250.0.

### Step 3: 6-iodo-3-methylimidazo[4,5-b]pyridine

To a stirred mixture of 5-iodo-N2-methylpyridine-2,3-diamine (1.30 g, 5.22 mmol) in CH₃COOH (13.00 mL) was added trimethyl orthoformate (1.11 g, 0.01 mmol) at room temperature under air atmosphere. The reaction mixture was stirred for 16 h at 70° C. under air atmosphere. The resulting mixture was concentrated under reduced pressure. The residue was diluted with EtOAc (100 mL). The mixture was basified to pH 8 with saturated NaHCO₃ (aq.). The resulting mixture was extracted with EtOAc (3×150 mL). The combined organic layers were washed with brine (200 mL), dried over anhydrous Na₂SO₄ and filtered. The filtrate was concentrated under reduced pressure. The residue was purified by silica gel column chromatography, eluted with CH₂Cl₂/MeOH (20:1). The fractions contained desired product were combined and concentrated to afford 6-iodo-3-methylimidazo[4,5-b]pyridine (0.85 g, 62%) as a brown solid. MS ESI calculated for C₇H₆IN₃ [M+H]⁺, 258.96; found 259.85.

### Step 4: 3-methyl-6-[2-(trimethylsilyl)ethynyl]imidazo[4,5-b]pyridine

To a stirred mixture of 6-iodo-3-methylimidazo[4,5-b]pyridine (0.8 g, 3.08 mmol), trimethylsilylacetylene (0.9 g, 9.26 mmol), Pd(PPh3)2Cl2 (0.22 g, 0.31 mmol) and CuI (0.12 g, 0.62 mmol) in DMF (8.00 mL) was added TEA (6.25 g, 61.76 mmol) dropwise at room temperature. The reaction mixture was degassed with argon for three times and stirred for 2 h at 80° C. The residue was diluted with water (150 mL), extracted with EtOAc (3×150 mL). The combined organic layers were washed with brine (200 mL), dried over anhydrous Na2SO4 and filtered. The filtrate was concentrated under reduced pressure. The residue was purified by silica gel column chromatography, eluted with CH2Cl2/MeOH (10:1). The fractions contained desired product were combined and concentrated to afford 3-methyl-6-[2-(trimethylsilyl)ethynyl]imidazo[4,5-b]pyridine (0.70 g, 98%) as a brown solid. MS ESI calculated for C12H15N3Si [M+H]+, 230.1; found 230.0.

### Step 5: 6-ethynyl-3-methylimidazo[4,5-b]pyridine

To a stirred mixture of 3-methyl-6-[2-(trimethylsilyl)ethynyl]imidazo[4,5-b]pyridine (0.56 g, 2.44 mmol) in THF (6.00 mL) was added TBAF (0.96 g, 3.66 mmol) at room temperature under air atmosphere. The reaction mixture was stirred for 16 h at room temperature. The resulting mixture was concentrated under reduced pressure. The residue was diluted with water (100 mL), extracted with EtOAc (3×150 mL). The combined organic layers were washed with brine (200 mL), dried over anhydrous Na2SO4 and filtered. The filtrate was concentrated under reduced pressure. The residue was purified by silica gel column chromatography, eluted with CH2Cl2/MeOH (10:1). The fractions contained desired product were combined and concentrated to afford 6-ethynyl-3-methylimidazo[4,5-b]pyridine (0.30 g, 78%) as a brown solid. MS ESI calculated for C9H7N3 [M+H]+, 158.06; found 158.10.

### Step 6: 1-[(3S,5R)-5-(methoxymethyl)-1-(prop-2-enoyl)pyrrolidin-3-yl]-5-(methylamino)-3-(2-[3-methylimidazo[4,5-b]pyridin-6-yl]ethynyl)pyrazole-4-carboxamide

To a stirred mixture of 3-bromo-1-[(3S,5R)-5-(methoxymethyl)-1-(prop-2-enoyl)pyrrolidin-3-yl]-5-(methylamino)pyrazole-4-carboxamide (0.2 g, 0.52 mmol), 6-ethynyl-3-methylimidazo[4,5-b]pyridine (0.12 g, 0.78 mmol), Pd(PPh₃)₂Cl₂ (36.34 mg, 0.05 mmol) and CuI (19.72 mg, 0.11 mmol) in DMF (2.00 mL) was added TEA (0.16 g, 1.55 mmol) dropwise at room temperature. The reaction mixture was degassed with argon for three times and stirred for 2 h at 90° C. The residue was purified by reverse flash chromatography with the following conditions: column, C18 silica gel; mobile phase ACN, NH₄HCO₃ 0.01 mmol in water, 20% to 40% gradient in 20 min; detector, UV 254 nm. The fractions contained desired product were combined and concentrated. The residue was purified by Prep-HPLC with the following conditions Column: SunFire Prep C18 OBD Column, 19×150 mm 5 um 10 nm; Mobile Phase A: Water (0.1% FA), Mobile Phase B: MeOH—HPLC; Flow rate: 20 mL/min; Gradient: 30 B to 50 B in 6 min; 210/254 nm; RT1: 5.68. The fractions contained desired product were combined and concentrated to offord 1-[(3S,5R)-5-(methoxymethyl)-1-(prop-2-enoyl)pyrrolidin-3-yl]-5-(methylamino)-3-(2-[3-methylimidazo[4,5-b]pyridin-6-yl]ethynyl)pyrazole-4-carboxamide (22.5 mg, 9%) as a light yellow solid. MS ESI calculated for C₂₃H₂₆N₈O₃ [M+H]⁺, 463.21; found 463.15. ¹H NMR (300 MHz, DMSO-d₆) δ 8.64-8.57 (m, 2H), 8.35 (d, J=1.8 Hz, 1H), 7.28 (s, 1H), 6.65-6.43 (m, 2H), 6.15-5.96 (m, 1H), 5.68-5.43 (m, 1H), 5.23-5.01 (m, 1H), 4.60-4.47 (m, 1H), 4.44-4.34 (m, 1H), 4.05-4.00 (m, 1H), 3.89-3.85 (m, 4H), 3.65-3.42 (m, 2H), 3.31-3.27 (m, 3H), 2.95-2.90 (m, 3H), 2.79-2.55 (m, 1H), 2.39-2.19 (m, 1H).

### Example 24: 1-((3S,5R)-1-acryloyl-5-(methoxymethyl)pyrrolidin-3-yl)-3-((6-cyano-1-ethyl-2-methyl-1H-benzo[d]imidazol-5-yl)ethynyl)-5-(methylamino)-1H-pyrazole-4-carboxamide

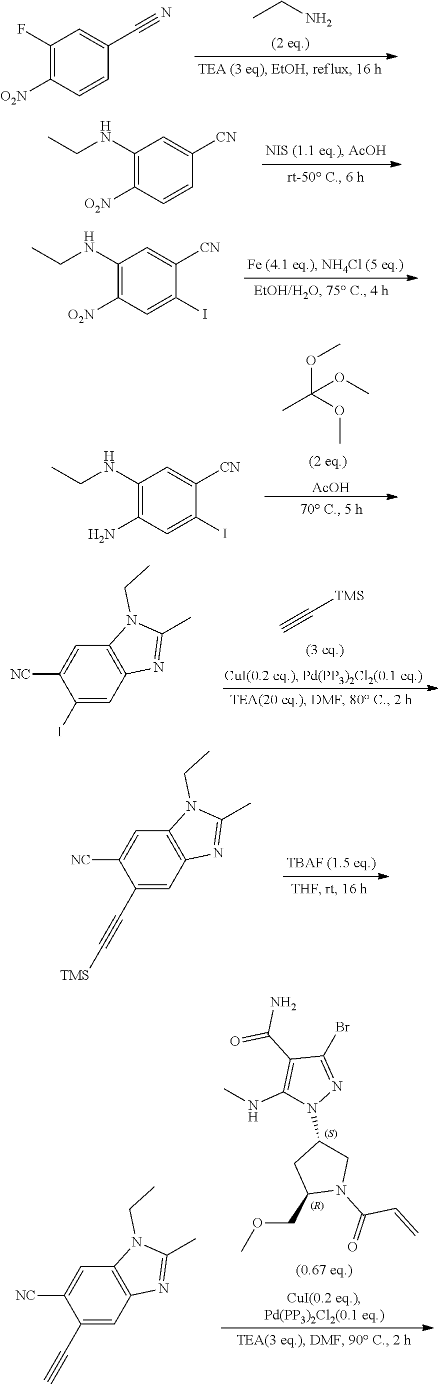

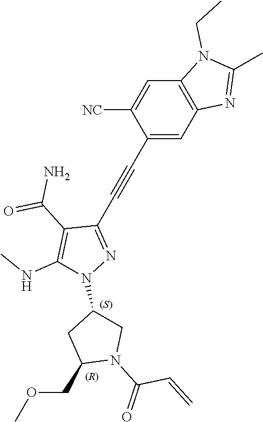

### Step 1: 3-(ethylamino)-4-nitrobenzonitrile

To a stirred mixture of 3-fluoro-4-nitrobenzonitrile (10.00 g, 60.20 mmol) and ethylamine (5.43 g, 120.40 mmol) in EtOH (90.00 mL) was added TEA (25.10 mL, 248.08 mmol) at room temperature under nitrogen atmosphere. The reaction mixture was stirred for 16 h at 50° C. The resulting mixture was concentrated under reduced pressure. The residue was diluted with NaHCO3 (300 mL). The resulting mixture was extracted with EtOAc (3×200 mL). The combined organic layers were washed with brine (300 mL), dried over anhydrous Na2SO4 and filtered. The filtrate was concentrated under reduced pressure. The residue was dried to offord 3-(ethylamino)-4-nitrobenzonitrile (11.5 g, 89%) which was used in the next step directly without further purification. H-NMR (400 MHz, Chloroform-d): δ 8.27 (d, J=8.7 Hz, 1H), 7.98 (s, 1H), 7.17 (d, J=1.7 Hz, 1H), 6.88 (dd, J=8.7, 1.7 Hz, 1H), 3.41-3.38 (m, 2H), 1.43 (t, J=7.2 Hz, 3H).

### Step 2: 5-(ethylamino)-2-iodo-4-nitrobenzonitrile

To a stirred solution of 3-(ethylamino)-4-nitrobenzonitrile (12.50 g, 65.38 mmol) in AcOH (120.00 mL) was added NIS (16.18 g, 71.92 mmol) in portions at room temperature under nitrogen atmosphere. The reaction mixture was stirred for 6 h at 50° C. The resulting mixture was concentrated under reduced pressure. The residue was diluted with EtOAc (100 mL). The resulting mixture was basified to pH 8 with saturated NaHCO₃ (aq.) and extracted with EtOAc (3×500 mL). The combined organic layers were washed with brine (3×300 mL), dried over anhydrous Na₂SO₄ and filtered. The filtrate was concentrated under reduced pressure. The residue was purified by trituration with CH₂Cl₂/MeOH (50:1) (500 mL). The precipitated solids were collected by filtration and dried to afford 5-(ethylamino)-2-iodo-4-nitrobenzonitrile (13.9 g, 60%) as a reddish brown solid. MS ESI calculated for C₉H₈IN₃O₂[M−H]⁻, 315.97, found 315.90.

### Step 3: 4-amino-5-(ethylamino)-2-iodobenzonitrile

To a stirred mixture of 5-(ethylamino)-2-iodo-4-nitrobenzonitrile (3.00 g, 9.46 mmol) and NH₄Cl (2.53 g, 47.31 mmol) in EtOH (30.00 mL) and H₂O (4.50 mL) was added Fe (2.11 g, 37.85 mmol). The reaction mixture was stirred for 4 h at 75° C. under nitrogen atmosphere. The resulting mixture was filtered, the filter cake was washed with EtOH (6×300 mL). The filtrate was concentrated under reduced pressure. The residue was diluted with water (300 mL), extracted with EtOAc (3×200 mL). The combined organic layers were washed with brine (300 mL), dried over anhydrous Na₂SO₄ and filtered. The filtrate was concentrated under reduced pressure. The crude product 4-amino-5-(ethylamino)-2-iodobenzonitrile (2.4 g, 79%) was used in the next step directly without further purification. MS ESI calculated for C₉H₁₀IN₃ [M−H]⁻, 285.99, found 286.00.

### Step 4: 3-ethyl-6-iodo-2-methyl-1,3-benzodiazole-5-carbonitrile

To a stirred solution of 4-amino-5-(ethylamino)-2-iodobenzonitrile (3.50 g 12.19 mmol) in AcOH (35.00 mL) was added 1,1,1-trimethoxyethane (2.93 g, 24.38 mmol) at room temperature under nitrogen atmosphere. The reaction mixture was stirred for 5 h at 70° C. The resulting mixture was concentrated under reduced pressure. The residue was diluted with EtOAc (100 mL). The mixture was basified to pH 8 with saturated NaHCO₃ (aq.). The resulting mixture was extracted with EtOAc (3×150 mL). The combined organic layers were washed with brine (200 mL), dried over anhydrous Na₂SO₄ and filtered. The filtrate was concentrated under reduced pressure. The residue was purified by silica gel column chromatography, eluted with CH₂Cl₂/MeOH (20:1). The fractions contained desired product were combined and concentrated to afford 3-ethyl-6-iodo-2-methyl-1,3-benzodiazole-5-carbonitrile (2.18 g, 54%) as an off-white solid. MS ESI calculated for C₁₁H₁₀IN₃ [M+H]⁺, 311.99, found 312.05.

### Step 5: 3-ethyl-2-methyl-6-[2-(trimethylsilyl)ethynyl]-1,3-benzodiazole-5-carbonitrile

To a stirred mixture of 3-ethyl-6-iodo-2-methyl-1,3-benzodiazole-5-carbonitrile (2.18 g, 7.01 mmol), trimethylsilylacetylene (2.97 mL, 30.25 mmol), CuI (0.27 g, 1.40 mmol) and Pd(PPh₃)₂Cl₂ (0.49 g, 0.70 mmol) in DMF (22.00 mL) was added TEA (19.48 mL, 192.50 mmol). The reaction mixture was degassed with nitrogen for three times and stirred for 2 h at 80° C. The resulting mixture was diluted with water (100 mL) and extracted with EA (3×100 mL). The combined organic layers were washed with brine (2×100 mL), dried over anhydrous Na₂SO₄, filtered and concentrated under reduced pressure. The residue was purified by silica gel column chromatography, eluted with CH₂Cl₂/MeOH (25:1). The fractions contained desired product were combined and concentrated to afford 3-ethyl-2-methyl-6-[2-(trimethylsilyl)ethynyl]-1,3-benzodiazole-5-carbonitrile (2 g, 94%) as a brown solid. MS ESI calculated for C₁₆H₁₉N₃Si [M+H]⁺, 282.13, found 282.15.

### Step 6: 3-ethyl-6-ethynyl-2-methyl-1,3-benzodiazole-5-carbonitrile

To a stirred solution of 3-ethyl-2-methyl-6-[2-(trimethylsilyl)ethynyl]-1,3-benzodiazole-5-carbonitrile (1.00 g, 3.55 mmol) in THF (10.00 mL) was added TBAF (5.33 mL, 5.33 mmol) dropwise at 0° C. under nitrogen atmosphere. The reaction mixture was stirred for 16 h at room temperature. The resulting mixture was diluted with water (80 mL), extracted with EtOAc (3×100 mL). The combined organic layers were washed with brine (150 mL), dried over anhydrous Na₂SO₄ and filtered. The filtrate was concentrated under reduced pressure. The residue was purified by silica gel column chromatography, eluted with CH₂Cl₂/MeOH (25:1). The fractions contained desired product were combined and concentrated to afford 3-ethyl-6-ethynyl-2-methyl-1,3-benzodiazole-5-carbonitrile (0.70 g, 84%) as a yellow solid. MS ESI calculated for C₁₃H₁₁N₃ [M+H]⁺, 210.10, found 210.20.

### Step 7: 3-[2-(6-cyano-1-ethyl-2-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a stirred mixture of 3-bromo-1-[(3S,5R)-5-(methoxymethyl)-1-(prop-2-enoyl)pyrrolidin-3-yl]-5-(methylamino)pyrazole-4-carboxamide (0.20 g, 0.52 mmol), 3-ethyl-6-ethynyl-2-methyl-1,3-benzodiazole-5-carbonitrile (0.16 g 0.78 mmol), CuI (19.72 mg, 0.10 mmol) and Pd(PPh₃)₂Cl₂ (36.34 mg, 0.05 mmol) in DMF (2.00 mL) was added TEA (0.22 mL, 2.13 mmol). The reaction mixture was degassed with argon for three times and stirred for 2 h at 90° C. The resulting mixture was concentrated under reduced pressure. The residue was purified by reverse flash chromatography with the following conditions: column, C18 silica gel; mobile phase, NH₄HCO₃ (10 mmol/L) in water, 10% to 50% gradient in 25 min; detector, UV 254 nm. The fractions contained desired product were combined and concentrated. The crude product was purified by Prep-HPLC with the following conditions Column: XBridge Prep OBD C18 Column, 19×250 mm, 5 um; Mobile Phase A: Water (10 mmol/L NH₄HCO₃), Mobile Phase B: ACN; Flow rate: 20 mL/min; Gradient: 25 B to 60 B in 5.8 min; 210/254 nm; RT1: 5.52. The fractions contained desired product were combined and concentrated to afford 3-[2-(6-cyano-1-ethyl-2-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (49.9 mg, 18%) as an off-white solid. MS ESI calculated for C₂₇H₃₀N₈O₃ [M+H]⁺, 515.24, found 515.30. H-NMR (400 MHz, DMSO-d₆): δ 8.34 (d, J=1.3 Hz, 1H), 7.98 (d, J=0.5 Hz, 1H), 7.43 (s, 1H), 6.78-6.53 (m, 3H), 6.19-6.17 (m, 1H), 5.73-5.61 (m, 1H), 5.33-5.28 (m, 1H), 4.50-4.45 (m, 1H), 4.35-4.30 (m, 2H), 4.07-3.70 (m, 2H), 3.63-3.43 (m, 2H), 3.30 (s, 3H), 2.96-2.92 (m, 3H), 2.63 (s, 3H), 2.50-2.47 (m, 1H), 2.36-2.27 (m, 1H), 1.32 (t, J=7.2 Hz, 3H).

### Example 25: 1-((3S,5R)-1-acryloyl-5-(methoxymethyl)pyrrolidin-3-yl)-3-((2-cyclopropyl-1-ethyl-1H-benzo[d]imidazol-5-yl)ethynyl)-5-(methylamino)-1H-pyrazole-4-carboxamide

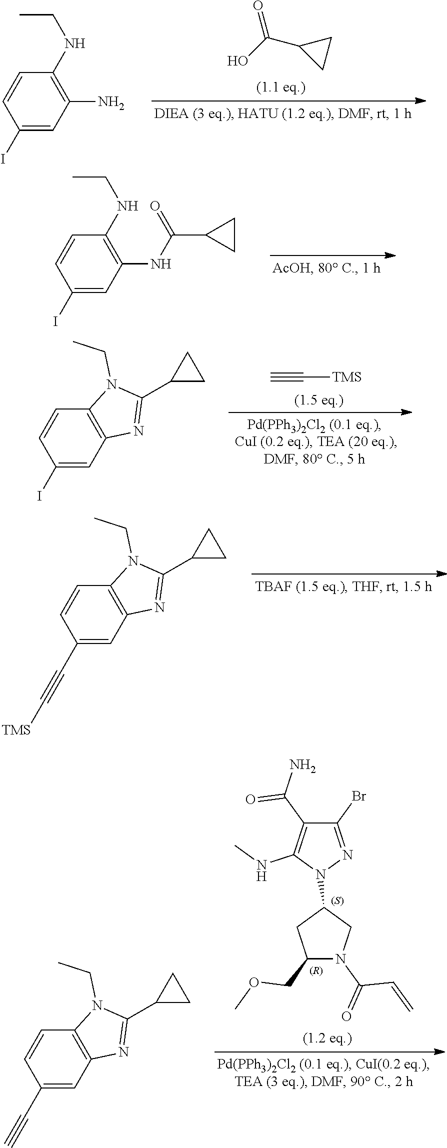

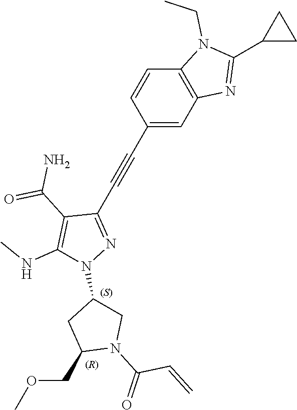

### Step 1: N-[2-(ethylamino)-5-iodophenyl]cyclopropanecarboxamide

To a stirred mixture of cyclopropanecarboxylic acid (0.16 g, 1.91 mmol) and HATU (1.09 g 2.86 mmol) in DMF (5.00 mL) was added N,N-diisopropylethylamine (0.74 g, 5.72 mmol) dropwise at room temperature. The reaction mixture was stirred for 10 min at room temperature. To the above mixture was added N1-ethyl-4-iodobenzene-1,2-diamine (0.50 g, 1.91 mmol) at room temperature. The resulting mixture was stirred for additional 1 h. The reaction was quenched with water (10 mL) and extracted with EtOAc (3×20 mL). The combined organic layers were washed with brine (3×10 mL), dried over anhydrous Na₂SO₄ and filtered. The filtrate was concentrated under reduced pressure to afford N-[2-(ethylamino)-5-iodophenyl]cyclopropanecarboxamide (0.50 g, 79%) as a dark grey solid which was used in the next step directly without further purification. ESI calculated for C₁₂H₁₅IN₂O [M+H]⁺, 331.02; found 331.05.

### Step 2: 2-Cyclopropyl-1-ethyl-5-iodo-1,3-benzodiazole

A mixture of N-[2-(ethylamino)-5-iodophenyl]cyclopropanecarboxamide (0.90 g, 2.73 mmol) in acetic acid (9.00 mL) was stirred for 1 h at 80° C. The resulting mixture was allowed to cool down to room temperature and concentrated under reduced pressure. The residue was neutralized to pH=7 with saturated NaHCO₃ (aq.) and extracted with EtOAc (3×50 mL). The combined organic layers were washed with brine (3×50 mL), dried over anhydrous Na₂SO₄ and filtered. The resulting mixture was concentrated under reduced pressure. The residue was purified by silica gel column chromatography, eluted with PE/EtOAc (3:1). The fractions contained desired product were combined and concentrated to afford 2-cyclopropyl-1-ethyl-5-iodo-1,3-benzodiazole (0.61 g, 71%) as a brown semi-solid. MS ESI calculated for C₁₂H₁₃IN₂ [M+H]⁺, 313.01; found 312.95.

### Step 3: 2-Cyclopropyl-1-ethyl-5-[2-(trimethylsilyl)ethynyl]-1,3-benzodiazole

To a mixture of 2-cyclopropyl-1-ethyl-5-iodo-1,3-benzodiazole (0.61 g, 1.95 mmol) and trimethylsilylacetylene (0.58 g, 5.86 mmol) in DMF (6.00 mL) were added Pd(PPh₃)₂Cl₂ (0.14 g, 0.19 mmol), CuI (74.43 mg, 0.39 mmol) and TEA (3.95 g, 39.08 mmol) at room temperature. The reaction mixture was degassed with nitrogen for three times and stirred for 5 h at 80° C. The mixture was allowed to cool down to room temperature. The resulting mixture was diluted with water (30 mL), extracted with EtOAc (3×50 mL). The combined organic layers were washed with brine (100 mL), dried over anhydrous Na₂SO₄ and filtered. The filtrate was concentrated under reduced pressure. The residue was purified by silica gel column chromatography, eluted with PE/EtOAc (3:1). The combined organic layers were washed with brine (300 mL), dried over anhydrous Na₂SO₄ and filtered. The filtrate was concentrated under reduced pressure to afford 2-cyclopropyl-1-ethyl-5-[2-(trimethylsilyl)ethynyl]-1,3-benzodiazole (0.54 g, 97%) as a light brown semi-solid. MS ESI calculated for C₁₇H₂₂N₂Si [M+H]⁺, 283.16; found 283.25.

### Step 4: 2-Cyclopropyl-1-ethyl-5-ethynyl-1,3-benzodiazole

To a stirred solution of 2-cyclopropyl-1-ethyl-5-[2-(trimethylsilyl)ethynyl]-1,3-benzodiazole (0.54 g, 1.91 mmol) in THF (6.00 mL) was added TBAF (0.75 g, 2.88 mmol). The reaction mixture was stirred for 1.5 h at room temperature. The reaction was quenched by the addition of water (5 mL) at room temperature. The resulting mixture was extracted with EtOAc (3×10 mL). The combined organic layers were washed with brine (3×10 mL), dried over anhydrous Na₂SO₄. After filtration, the filtrate was concentrated under reduced pressure. The residue was purified by silica gel column chromatography, eluted with PE/EtOAc (3:1). The fractions contained desired product were combined and concentrated to afford 2-cyclopropyl-1-ethyl-5-ethynyl-1,3-benzodiazole (0.35 g, 87%) as a pink semi-solid. MS ESI calculated for C₁₄H₁₄N₂[M+H]⁺, 211.12; found 211.05.

### Step 5: 3-[2-(2-Cyclopropyl-1-ethyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a mixture of 2-cyclopropyl-1-ethyl-5-ethynyl-1,3-benzodiazole (0.16 g 0.76 mmol) and 3-bromo-1-[(3S,5R)-5-(methoxymethyl)-1-(prop-2-enoyl)pyrrolidin-3-yl]-5-(methylamino)pyrazole-4-carboxamide (0.35 g, 0.91 mmol) in DMF (2.00 mL) were added Pd(PPh₃)₂Cl₂ (53.41 mg, 0.07 mmol), CuI (28.98 mg, 0.15 mmol) and TEA (0.23 g, 2.28 mmol) at room temperature. The reaction mixture was degassed with argon for three times and stirred for 2 h at 90° C. The resulting mixture was allowed to cool down to room temperature. The resulting mixture was extracted with EtOAc (3×10 mL). The combined organic layers were washed with brine (3×10 mL), dried over anhydrous Na₂SO₄ and filtered. The filtrate was concentrated under reduced pressure. The residue was purified by silica gel column chromatography, eluted with PE/EtOAc (1:1). The crude product (150 mg) was purified by Prep-HPLC with the following conditions Column: XBridge Prep C18 OBD Column, 19×150 mm 5 um; Mobile Phase A: Water (10 mmol/L NH₄HCO₃), Mobile Phase B: ACN; Flow rate: 20 mL/min; Gradient: 15 B to 60 B in 4.3 min; 254 nm; RT1:4.02. The fractions contained desired product were combined and concentrated to afford 3-[2-(2-cyclopropyl-1-ethyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (75.8 mg, 19%) as an off-white solid. MS ESI calculated for C₂₈H₃₃N₇O₃ [M+H]⁺, 516.26; found 516.15. ¹H NMR (300 MHz, DMSO-d₆) δ 7.69 (d, J=1.4 Hz, 1H), 7.57 (d, J=8.3 Hz, 1H), 7.38-7.31 (m, 2H), 6.85-6.48 (m, 3H), 6.16 (d, J=16.6 Hz, 1H), 5.68 (d, J=10.3 Hz, 1H), 5.31-5.13 (m, 1H), 4.41-4.34 (m, 3H), 3.87-3.84 (m, 1H), 3.79-3.66 (m, 1H), 3.61-3.57 (m, 1H), 3.50-3.37 (m, 2H), 3.30-3.24 (m, 3H), 2.95-2.90 (m, 3H), 2.44-2.16 (m, 2H), 1.34 (t, J=7.1 Hz, 3H), 1.10-1.05 (m, 4H).

### Example 26: 1-((3S,5R)-1-acryloyl-5-(methoxymethyl)pyrrolidin-3-yl)-3-(benzo[d]isoxazol-6-ylethynyl)-5-(methylamino)-1H-pyrazole-4-carboxamide

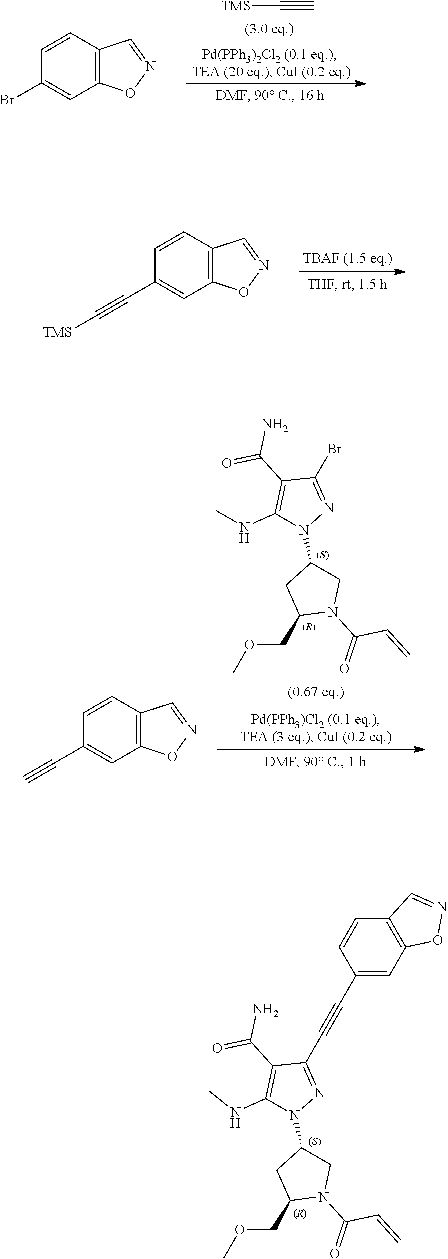

### Step 1: 6-[2-(trimethylsilyl)ethynyl]-1,2-benzoxazole

To a mixture of 6-bromo-1,2-benzoxazole (2.00 g, 10.10 mmol), trimethylsilylacetylene (1.49 g, 15.15 mmol), CuI (0.38 g, 2.02 mmol) and Pd(PPh₃)₂Cl₂ (0.71 g, 1.01 mmol) in DMF (8.00 mL) was added TEA (20.44 g, 202.00 mmol). The reaction mixture was degassed with argon for three times and stirred for 16 h at 90° C. The resulting mixture was diluted with water (100 mL). The resulting mixture was extracted with EtOAc (3×150 mL). The combined organic layers were washed with water (3×100 mL), dried over anhydrous Na₂SO₄ and filtered. The filtrate was concentrated under reduced pressure. The residue was purified by silica gel column chromatography, eluted with PE/EtOAc (1:1). The fractions contained desired product were combined and concentrated to afford 6-[2-(trimethylsilyl)ethynyl]-1,2-benzoxazole (0.80 g, 36%) as a yellow semi-solid. MS ESI calculated for C₁₂H₁₃NOSi [M−H]⁻, 214.09, found 214.15.

### Step 2: 6-ethynyl-1,2-benzoxazole

To a stirred solution of 6-[2-(trimethylsilyl)ethynyl]-1,2-benzoxazole (0.80 g, 3.72 mmol) in THF (8.00 mL) was added TBAF (5.57 mL, 5.57 mmol) dropwise at 0° C. under air atmosphere. The reaction mixture was stirred for 1.5 h at room temperature. The resulting mixture was concentrated under reduced pressure. The residue was purified by silica gel column chromatography, eluted with PE/EtOAc (1:1). The fractions contained desired product were combined and concentrated to afford 6-ethynyl-1,2-benzoxazole (0.29 g, 54%) as a light brown solid. MS ESI calculated for C₉HsNO [M−H]⁻, 142.04, found 142.10.

### Step 3: 3-[2-(1,2-benzoxazol-6-yl)ethynyl]-1-[(3S,5R)-5-(methoxymethyl)-1-(prop-2-enoyl)pyrrolidin-3-yl]-5-(methylamino)pyrazole-4-carboxamide

To a stirred mixture of 3-bromo-1-[(3S,5R)-5-(methoxymethyl)-1-(prop-2-enoyl)pyrrolidin-3-yl]-5-(methylamino)pyrazole-4-carboxamide (0.20 g, 0.52 mmol) and 6-ethynyl-1,2-benzoxazole (0.12 g 0.78 mmol) in DMF (2.00 mL) was added CuI (19.72 mg, 0.10 mmol), Pd(PPh₃)₂Cl₂ (36.34 mg, 0.05 mmol) and TEA (0.16 g, 1.55 mmol). The reaction mixture was degassed with argon for three times and stirred for 1 h at 90° C. The resulting mixture was purified by reverse flash chromatography with the following conditions: column, C18 silica gel; mobile phase, ACN in water (10 mM NH₄HCO₃), 5% to 35% gradient in 30 min; detector, UV 220 nm. The crude product (150 mg) was purified by Prep-HPLC with the following conditions Column: XBridge Prep OBD C18 Column, 19*250 mm, 5 um; Mobile Phase A: Water (10 mmol/L NH₄HCO₃), Mobile Phase B: ACN; Flow rate: 20 mL/min; Gradient: 30 B to 60 B in 5.8 min; 210/254 nm; RT1:5.65. The fractions contained desired product were combined and concentrated to afford 3-[2-(1,2-benzoxazol-6-yl)ethynyl]-1-[(3S,5R)-5-(methoxymethyl)-1-(prop-2-enoyl)pyrrolidin-3-yl]-5-(methylamino)pyrazole-4-carboxamide (37.00 mg, 16%) as a white solid. MS ESI calculated for C₂₃H₂₄N₆O₄ [M+H]⁺, 449.19, found 449.25. H-NMR (400 MHz, DMSO-d₆): δ 7.67 (d, J=7.9 Hz, 1H), 7.34 (m, 1H), 7.08 (d, J=13.3 Hz, 2H), 6.71 (s, 1H), 6.58 (m, J=16.8, 10.3 Hz, 1H), 6.52 (d, J=5.7 Hz, 1H), 6.17 (d, J=16.6 Hz, 1H), 5.69 (d, J=10.0 Hz, 1H), 5.30-5.14 (m, 1H), 4.52 (s, 1H), 4.39 (s, 1H), 4.06-3.97 (m, 1H), 3.89-3.79 (m, 1H), 3.77-3.73 (m, 1H), 3.61-3.58 (m, 1H), 3.48-3.45 (m, 3H), 2.94 (s, 3H), 2.53-2.49 (m, 1H), 2.36-2.32 (m, 1H).

### Example 27: 1-((3S,5R)-1-acryloyl-5-(methoxymethyl)pyrrolidin-3-yl)-3-((1-ethyl-2-(trifluoromethyl)-1H-benzo[d]imidazol-5-yl)ethynyl)-5-(methylamino)-1H-pyrazole-4-carboxamide

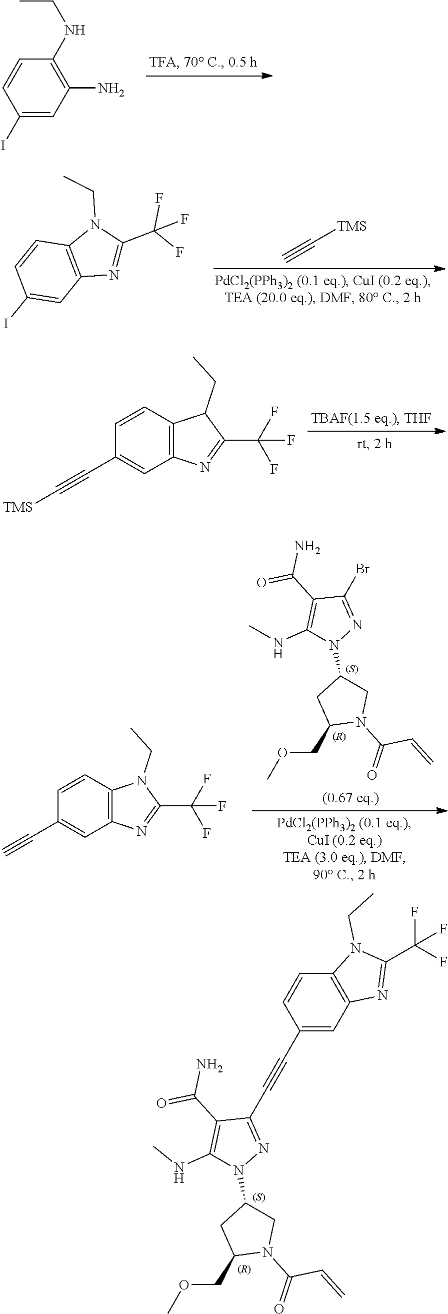

### Step 1: 1-ethyl-5-iodo-2-(trifluoromethyl)-1H-benzo[d]imidazole

A solution of N¹-ethyl-4-iodobenzene-1,2-diamine (2.00 g, 7.63 mmol) in CF₃COOH was stirred for 0.5 h at 70° C. under nitrogen atmosphere. The resulting mixture was concentrated under reduced pressure. The residue was diluted with EA (50 mL), quenched by the addition of sat. NH₃HCO₃ (aq.) (50 mL) at room temperature. The resulting mixture was extracted with EtOAc (3×150 mL). The combined organic layers were washed with brine (300 mL), dried over anhydrous Na₂SO₄ and filtered. The resulting mixture was concentrated under reduced pressure. The residue was purified by silica gel column chromatography, eluted with PE/EtOAc (5:1). The fractions contained desired product were combined and concentrated to afford 1-ethyl-5-iodo-2-(trifluoromethyl)-1H-benzo[d]imidazole (2.5 g, 96%) as an off-white solid. ESI calculated for C₁₀H₈F₃IN₂ [M+H]⁺, 340.97; found 340.80.

### Step 2: 3-ethyl-2-(trifluoromethyl)-6-((trimethylsilyl)ethynyl)-3H-indole

To a stirred mixture of 1-ethyl-5-iodo-2-(trifluoromethyl)-1H-benzo[d]imidazole (1.25 g, 3.68 mmol), Pd(PPh₃)₂Cl₂ (0.26 g, 0.37 mmol), CuI (0.14 g, 0.74 mmol) and trimethylsilylacetylene (1.08 g, 11.03 mmol) in DMF was added TEA (7.44 g, 73.51 mmol). The reaction mixture was degassed with argon for three times and stirred for 2 h at 80′° C. The resulting mixture was extracted with EtOAc (3×150 mL). The combined organic layers were washed with brine (200 mL), dried over anhydrous Na₂SO₄ and filtered. The resulting mixture was concentrated under reduced pressure. The residue was purified by silica gel column chromatography, eluted with PE/EtOAc (5:1). The fractions contained desired product were combined and concentrated to afford 3-ethyl-2-(trifluoromethyl)-6-((trimethylsilyl)ethynyl)-3H-indole (1.2 g, 96%) as a white solid. MS ESI calculated for C₁₆H₁₈F₃NSi [M+H]⁺, 310.12; found 310.25.

### Step 3: 1-ethyl-5-ethynyl-2-(trifluoromethyl)-1H-benzo[d]imidazole

To a stirred solution of 3-ethyl-2-(trifluoromethyl)-6-((trimethylsilyl)ethynyl)-3H-indole (1.10 g, 3.54 mmol) in THF was added TBAF (1.39 g, 5.32 mmol) dropwise at 0° C. under nitrogen atmosphere. The reaction mixture was stirred for 2 h at room temperature. The resulting mixture was concentrated under reduced pressure. The residue was purified by silica gel column chromatography, eluted with PE/EtOAc (5:1). The fractions contained desired product were combined and concentrated to afford 1-ethyl-5-ethynyl-2-(trifluoromethyl)-1H-benzo[d]imidazole (0.83 g, 98%) as a light yellow solid. MS ESI calculated for C₁₂H₉F₃N₂[M+H]⁺, 239.07; found 239.15.

### Step 4: 1-((3S,5R)-1-acryloyl-5-(methoxymethyl)pyrrolidin-3-yl)-3-((1-ethyl-2-(trifluoromethyl)-1H-benzo[d]imidazol-5-yl)ethynyl)-5-(methylamino)-1H-pyrazole-4-carboxamide

To a stirred mixture of 1-ethyl-5-ethynyl-2-(trifluoromethyl)-1H-benzo[d]imidazole (0.17 g, 0.70 mmol), Pd(PPh₃)₂Cl₂ (32.71 mg, 0.05 mmol), CuI (17.75 mg, 0.09 mmol) and 1-((3S,5R)-1-acryloyl-5-(methoxymethyl)pyrrolidin-3-yl)-3-bromo-5-(methylamino)-1H-pyrazole-4-carboxamide (0.18 g, 0.47 mmol) in DMF was added TEA (0.14 g, 1.40 mmol). The reaction mixture was degassed with argon for three times and stirred for 2 h at 90° C. The resulting mixture was concentrated under reduced pressure. The residue was purified by reverse flash chromatography with the following conditions: column, C18 silica gel; mobile phase, NH₄HCO₃ in water, 10% to 50% gradient in 10 min; detector, UV 254 nm. The crude product was purified by Prep-HPLC with the following conditions Column: Xselect CSH OBD Column 30*150 mm 5 um, Mobile Phase A: Water (10 mmol/L NH₄HCO₃), Mobile Phase B: ACN; Flow rate: 30 mL/min; Gradient: 20 B to 60 B in 6 min; 210/254 nm; RT: 5.59. The fractions contained desired product were combined and concentrated to afford 1-((3S,5R)-1-acryloyl-5-(methoxymethyl)pyrrolidin-3-yl)-3-((1-ethyl-2-(trifluoromethyl)-1H-benzo[d]imidazol-5-yl)ethynyl)-5-(methylamino)-1H-pyrazole-4-carboxamide (97.3 mg, 38%) as a white solid. MS ESI calculated for C₂₆H₂₈F₃N₇O₃ [M+H]⁺, 544.22; found 544.25. H-NMR (300 MHz, DMSO-d₆) δ 8.11 (s, 1H), 7.93 (d, J=8.6 Hz, 1H), 7.75-7.55 (m, 1H), 7.35 (s, 1H), 6.98-6.51 (m, 3H), 6.18 (d, J=16.6 Hz, 1H), 5.70 (d, J=10.4 Hz, 1H), 5.26 (s, 1H), 4.35-4.60 (m, 3H), 4.14-3.66 (m, 2H), 3.65-3.39 (m, 2H), 3.31-3.27 (m, 3H), 2.95 (t, J=4.4 Hz, 3H), 2.50-2.46 (m, 1H), 2.25-2.58 (m, 1H), 1.41 (t, J=7.1 Hz, 3H).

### Example 28: 1-((3S,5R)-1-acryloyl-5-(methoxymethyl)pyrrolidin-3-yl)-3-((6-fluoro-1,2-dimethyl-1H-benzo[d]imidazol-5-yl)ethynyl)-5-(methylamino)-1H-pyrazole-4-carboxamide

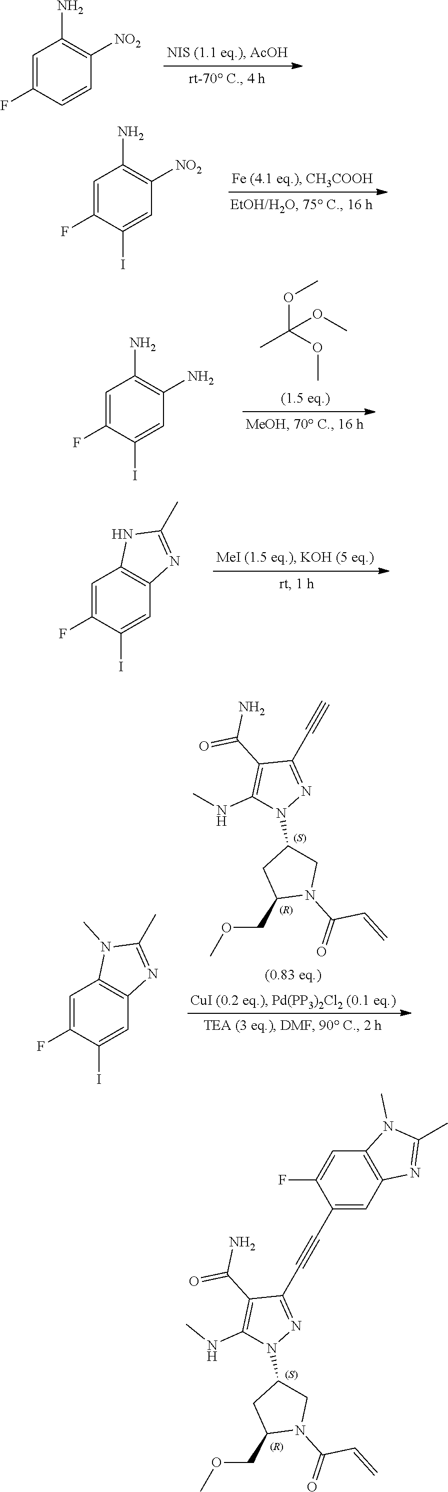

### Step 1: 5-fluoro-4-iodo-2-nitroaniline

To a mixture of 5-fluoro-2-nitroaniline (15.00 g, 96.08 mmol) in AcOH (120.00 mL) was added NIS (22.70 g, 100.88 mmol). The reaction mixture was stirred for 4 h at 70° C. The resulting mixture was concentrated under reduced pressure. The residue was diluted with EtOAc (50 mL). The mixture was basified to pH 8 with saturated NaHCO₃ (aq.). The resulting mixture was extracted with EtOAc (3×200 mL). The combined organic layers were washed with brine (300 mL), dried over anhydrous Na₂SO₄ and filtered. The filtrate was concentrated under reduced pressure. The residue was purified by silica gel column chromatography, eluted with PE/EtOAc (9:1). The fractions contained desired product were combined and concentrated to afford 5-fluoro-4-iodo-2-nitroaniline (24 g, 79%) as a yellow solid. MS ESI calculated for C₆H₄FIN₂O₂[M−H]⁻, 280.93; found 281.00.

### Step 2: 4-fluoro-5-iodobenzene-1,2-diamine

To a stirred mixture of 5-fluoro-4-iodo-2-nitroaniline (10.00 g, 35.46 mmol) and NH₄Cl (9.48 g, 177.29 mmol) in EtOH (100.00 mL) and H₂O (15.00 mL) was added Fe (8.12 g, 145.38 mmol). The reaction mixture was stirred for 16 h at 75° C. under nitrogen atmosphere. The resulting mixture was filtered, the filter cake was washed with ethanol (3×500 mL). The filtrate was concentrated under reduced pressure. The residue was diluted with water (200 mL). The resulting mixture was extracted with EtOAc (3×200 mL). The combined organic layers were washed with brine (500 mL), dried over anhydrous Na₂SO₄ and filtered. The filtrate was concentrated under reduced pressure and dried to offord 4-fluoro-5-iodobenzene-1,2-diamine (10 g, 89%) as a black solid which was used in the next step directly without further purification. MS ESI calculated for C₆H₆FIN₂ [M+H]⁺, 252.96, found 253.00.

### Step 3: 5-fluoro-6-iodo-2-methyl-3H-1,3-benzodiazole

To a solution of 4-fluoro-5-iodobenzene-1,2-diamine (3.17 g, 12.58 mmol) and in MeOH (30 mL) was added 1,1,1-trimethoxyethane (2.27 g, 18.87 mmol). The reaction mixture was stirred for overnight at 70° C. under nitrogen atmosphere. The resulting mixture was concentrated under reduced pressure. The residue was purified by silica gel column chromatography, eluted with CH₂Cl₂/MeOH (10:1). The fractions contained desired product were combined and concentrated to afford 5-fluoro-6-iodo-2-methyl-3H-1,3-benzodiazole (2.9 g, 83%) as a light yellow solid. MS ESI calculated for C₈H₆FIN₂ [M+H]⁺, 276.96, found 277.00.

### Step 4: 6-fluoro-5-iodo-1,2-dimethyl-1,3-benzodiazole

To a stirred mixture of 5-fluoro-6-iodo-2-methyl-3H-1,3-benzodiazole (1 g, 3.62 mmol) and KOH (1.01 g, 18.00 mmol) in acetone was added methyl iodide (0.36 mL) dropwise at 0° C. under nitrogen atmosphere. The reaction mixture was stirred for 1 h at room temperature. The resulting mixture was filtered, the filter cake was washed with ethyl acetate (3×10 mL). The filtrate was concentrated under reduced pressure. The residue was purified by silica gel column chromatography, eluted with CH₂Cl₂/MeOH (10:1). The fractions contained desired product were combined and concentrated. The residue was purified by Prep-SFC with the following conditions Column: CHIRALPAK IF, 5*25 cm, 5 um; Mobile Phase A: CO₂, Mobile Phase B: MeOH (0.1% 2 M NH₃-MEOH); Flow rate: 180 m/min; Gradient: 40% B; 220 nm; RT1:5.22; RT2:6.2. The fractions contained desired product were combined and concentrated to afford 6-fluoro-5-iodo-1,2-dimethyl-1,3-benzodiazole (0.47 g, 44%) as an off-white solid. MS ESI calculated for C₉H₈FIN₂ [M+H]⁺, 476.23, found 476.25.

### Step 5: 3-[2-(6-fluoro-1,2-dimethyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a stirred mixture of 3-ethynyl-1-[(3S,5R)-5-(methoxymethyl)-1-(prop-2-enoyl)pyrrolidin-3-yl]-5-(methylamino)pyrazole-4-carboxamide (0.15 g, 0.45 mmol), 6-fluoro-5-iodo-1,2-dimethyl-1,3-benzodiazole (0.16 g, 0.54 mmol), Pd(PPh₃)₂Cl₂ (31.77 mg, 0.05 mmol) and CuI (17.24 mg, 0.09 mmol) in DMF (1.50 mL, 20.52 mmol) was added TEA (0.19 mL, 1.86 mmol). The reaction mixture was degassed with argon for three times and stirred for 2 h at 90° C. The residue was purified by Prep-HPLC with the following conditions Column: XBridge Prep OBD C18 Column, 19*250 mm, 5 um; Mobile Phase A: Water (10 mmol/L NH₄HCO₃), Mobile Phase B: ACN; Flow rate: 20 mL/min; Gradient: 50 B to 80 B in 4.3 min; 210/254 nm; RT1: 4.03. The fractions contained desired product were combined and concentrated. The crude product (70 mg) was purified by Prep-HPLC with the following conditions Column: XBridge Prep C18 OBD Column, 19×150 mm 5 um; Mobile Phase A: Water (10 mmol/L NH₄HCO₃), Mobile Phase B: ACN; Flow rate: 20 mL/min; Gradient: 25 B to 50 B in 4.3 min; 210/254 nm. The fractions contained desired product were combined and concentrated to afford 3-[2-(6-fluoro-1,2-dimethyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (58 mg, 25%) as an off-white solid. MS ESI calculated for C₂₅H₂₈FN₇O₃[M+H]⁺, 494.22, found 494.30. H-NMR (400 MHz, DMSO-d₆): δ 7.78 (dd, J=6.3, 1.2 Hz, 1H), 7.61 (d, J=9.9 Hz, 1H), 7.49 (s, 1H), 6.79-6.52 (m, 3H), 6.18-6.16 (m, 1H), 5.72-5.67 (m, 1H), 5.28-5.25 (m, 1H), 4.55-4.42 (m, 1H), 4.06-3.56 (m, 5H), 3.52-3.42 (m, 2H), 3.35-3.28 (m, 3H), 2.99-2.93 (m, 3H), 2.53 (s, 3H), 2.49-2.43 (m, 1H), 2.35-2.27 (m, 1H).

### Example 29: 1-((3S,5R)-1-acryloyl-5-(methoxymethyl)pyrrolidin-3-yl)-3-((1-ethyl-4,6-difluoro-1H-benzo[d]imidazol-5-yl)ethynyl)-5-(methylamino)-1H-pyrazole-4-carboxamide

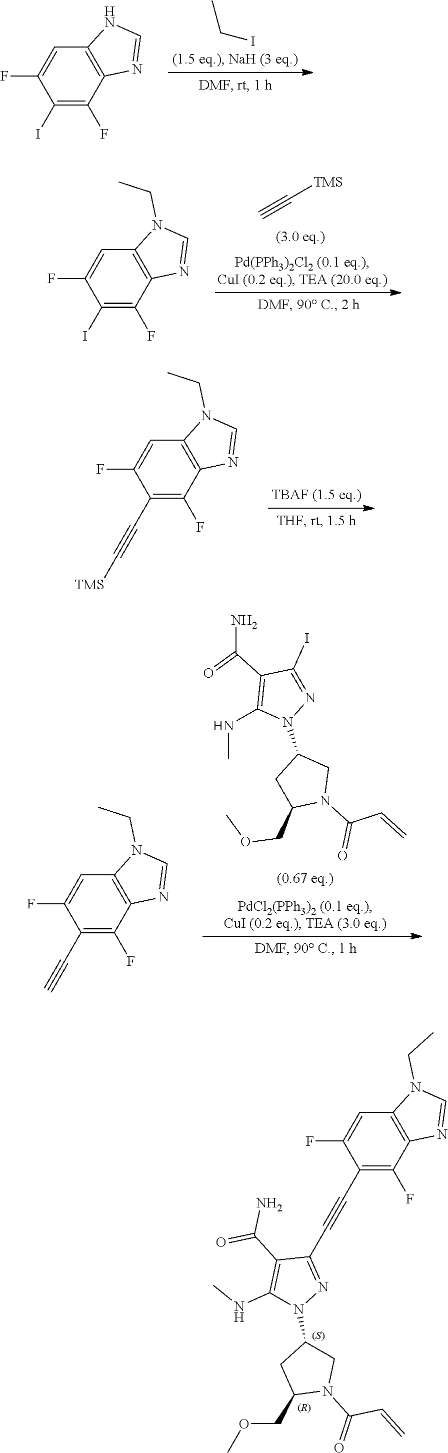

### Step 1: 1-ethyl-4,6-difluoro-5-iodo-1,3-benzodiazole

To a solution of 4,6-difluoro-5-iodo-1H-1,3-benzodiazole (1.00 g, 0.004 mmol) in DMF was added sodium hydride (60% in oil, 0.26 g) at 0° C. The mixture was stirred for 15 min. To the above mixture was added ethyl iodide (1.67 g, 0.01 mmol). The reaction mixture was stirred for 1 h at rt. The resulting mixture was quenched by water and extracted with EA (3×50 mL). The combined organic layers was dried over anhydrous Na₂SO₄ and filtered. The filtrate was concentrated under reduced pressure. The residue was purified by silica gel column chromatography, eluted with CH₂Cl₂/EtOAc (5:1). The fractions contained desired product were combined and concentrated to afford 1-ethyl-4,6-difluoro-5-iodo-1,3-benzodiazole (0.4 g, 36%) as an off-white solid. MS ESI calculated for C₉H₇F₂IN₂ [M+H]⁺, 308.96, found 308.90.

### Step 2: 1-ethyl-4,6-difluoro-5-[2-(trimethylsilyl)ethynyl]-1,3-benzodiazole

To a mixture of 1-ethyl-4,6-difluoro-5-iodo-1,3-benzodiazole (0.40 g, 1.29 mmol), trimethylsilylacetylene (0.38 g, 3.90 mmol), CuI (49.46 mg, 0.26 mmol) and Pd(PPh₃)₂Cl₂ (91.14 mg, 0.13 mmol) in DMF (4.00 mL) was added TEA (2.63 g, 25.97 mmol). The reaction mixture was degassed with argon for three times and stirred for 2 h at 90° C. The resulting mixture was diluted with water (50 mL), extracted with EtOAc (3×50 mL). The combined organic layers were washed with water (3×30 mL), dried over anhydrous Na₂SO₄ and filtered. The filtrate was concentrated under reduced pressure. The residue was purified by silica gel column chromatography, eluted with PE/EtOAc (1:1). The fractions contained desired product were combined and concentrated to afford 1-ethyl-4,6-difluoro-5-[2-(trimethylsilyl)ethynyl]-1,3-benzodiazole (0.30 g, 83%) as a yellow oil. MS ESI calculated for C₁₀H₆F₂N₂ [M+H]⁺, 279.11, found 279.25.

### Step 3: 1-ethyl-5-ethynyl-4,6-difluoro-1,3-benzodiazole

To a stirred solution of 1-ethyl-4,6-difluoro-5-[2-(trimethylsilyl)ethynyl]-1,3-benzodiazole (0.30 g, 1.08 mmol) in THF (3.00 mL) was added TBAF (1.62 mL, 1.62 mmol) dropwise at 0° C. under air atmosphere. The reaction mixture was stirred for 1.5 h at room temperature. The resulting mixture was concentrated under reduced pressure. The residue was purified by silica gel column chromatography, eluted with PE/EtOAc (1:1). The fractions contained desired product were combined and concentrated to afford 1-ethyl-5-ethynyl-4,6-difluoro-1,3-benzodiazole (0.18 g, 81%) as a light yellow solid. MS ESI calculated for C₁₁H₈F₂N₂[M+H]⁺, 207.07, found 207.05.

### Step 4: 3-[2-(1-ethyl-4,6-difluoro-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a stirred mixture of 3-iodo-1-[(3S,5R)-5-(methoxymethyl)-1-(prop-2-enoyl)pyrrolidin-3-yl]-5-(methylamino)pyrazole-4-carboxamide (0.15 g, 0.35 mmol), 1-ethyl-5-ethynyl-4,6-difluoro-1,3-benzodiazole (0.14 g 0.692 mmol), Pd(PPh₃)₂Cl₂ (24.30 mg, 0.04 mmol) and CuI (13.19 mg, 0.069 mmol) in DMF (2.00 mL) was added TEA (0.11 g, 1.04 mmol). The reaction mixture was degassed with argon for three times and stirred for 1 h at 90° C. The resulting mixture was purified by reverse flash chromatography with the following conditions: column, C18 silica gel; mobile phase, ACN in water (10 mM NH₄HCO₃), 5% to 35% gradient in 30 min; detector, UV 254 nm. The fractions contained desired product were combined and concentrated. The crude product (130 mg) was purified by Prep-HPLC with the following conditions Column: XBridge Prep OBD C18 Column, 19*250 mm, 5 um; Mobile Phase A: Water (10 mmol/L NH₄HCO₃), Mobile Phase B: ACN; Flow rate: 20 mL/min; Gradient: 30 B to 60 B in 5.8 min; 210/254 nm; RT1:5.56. The fractions contained desired product were combined and concentrated to afford 3-[2-(1-ethyl-4,6-difluoro-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (91 mg, 51%) as a white solid. MS ESI calculated for C₂₅H₂₇F₂N₇O₃[M+H]⁺, 512.21, found 512.20. H-NMR (400 MHz, DMSO-d₆): δ 8.46 (s, 1H), 7.73-7.70 (m, 1H), 7.58 (s, 1H), 6.78-6.55 (m, 3H), 6.18-6.25 (m, 1H), 5.71-5.68 (m, 1H), 5.29-5.26 (m, 1H), 4.60-4.36 (m, 1H), 4.30 (q, J=7.3 Hz, 2H), 4.08-3.69 (m, 2H), 3.65-3.41 (m, 2H), 3.35-3.30 (m, 3H), 2.97 (t, J=5.3 Hz, 3H), 2.71-2.58 (m, 1H), 2.33-2.30 (m, 1H), 1.41 (t, J=7.2 Hz, 3H).

### Example 30: 1-((3S,5R)-1-acryloyl-5-(methoxymethyl)pyrrolidin-3-yl)-3-((1-(difluoromethyl)-4,6-difluoro-1H-benzo[d]imidazol-5-yl)ethynyl)-5-(methylamino)-1H-pyrazole-4-carboxamide

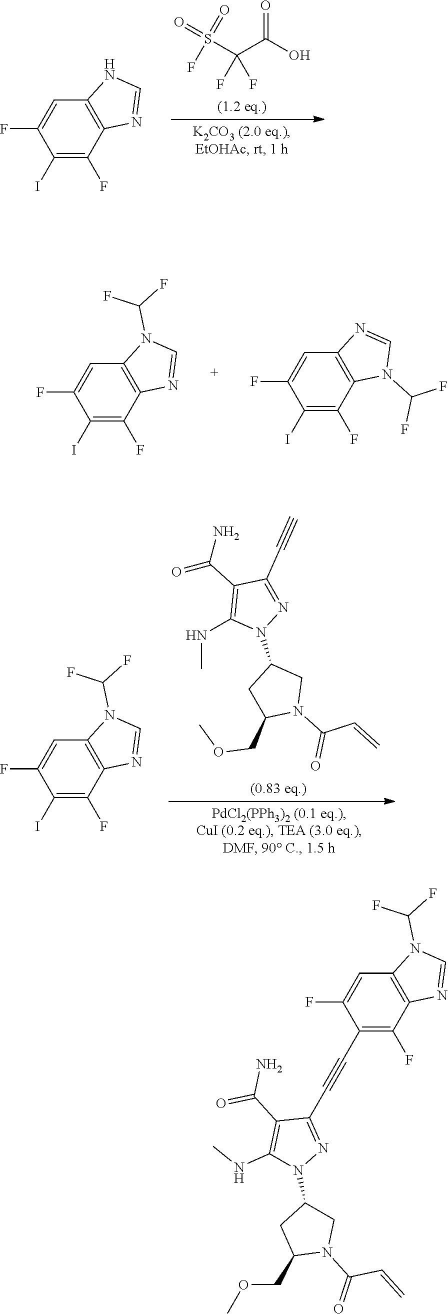

### Step 1: 1-(difluoromethyl)-4,6-difluoro-5-iodo-1,3-benzodiazole

To a stirred solution of 4,6-difluoro-5-iodo-1H-1,3-benzodiazole (1.00 g, 3.57 mmol) in EA (8.00 mL) were added difluoro(sulfo)acetic acid (0.76 g, 4.28 mmol) and K₂CO₃ (1.48 g, 10.71 mmol) at room temperature. The reaction mixture was stirred at room temperature for 1 h. The resulting mixture was quenched by saturated aqueous sodium bicarbonate (30 mL). The organic layer was separated and the aqueous layer was extracted with EA (3×45 mL). The combined organic layers were washed with brine (2×45 mL), dried over anhydrous sodium sulfate and filtered. The filtrate was concentrated under reduced pressure. The residue was purified by silica gel column chromatography, eluted with EA in DCM (0-50%). The fractions contained desired product were combined and concentrated. The crude product was further separated by Prep-Achiral-SFC with the following conditions Column: BEH 2-Ethylpyridine, 30*150 mm, 5 μm; Mobile Phase A: CO₂, Mobile Phase B: MeOH (0.1% 2 M NH₃-MeOH); Flow rate: 45 mL/min; Gradient: 10% B; Detector: UV 254 nm). The faster fractions (RT: 2.93 min) contained the desired product were combined and concentrated under reduced pressure to afford 1-(difluoromethyl)-4,6-difluoro-5-iodo-1,3-benzodiazole (0.45 g, 37%) as a white solid. The slower fractions (RT: 3.50 min) contained the desired product were combined and concentrated under reduced pressure to afford 1-(difluoromethyl)-5,7-difluoro-6-iodo-1,3-benzodiazole (0.15 g, 12%) as a white solid. MS ESI calculated for C₈H₃F₄IN₂ [M+H]⁺, 330.93, found 330.95.

### Step 2: 3-[2-[1-(difluoromethyl)-4,6-difluoro-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide

To a stirred mixture of 1-(difluoromethyl)-4,6-difluoro-5-iodo-1,3-benzodiazole (0.18 g, 0.54 mmol) and 3-ethynyl-1-[(3S,5R)-5-(methoxymethyl)-1-(prop-2-enoyl)pyrrolidin-3-yl]-5-(methylamino)pyrazole-4-carboxamide (0.15 g, 0.45 mmol) in DMF (4.00 mL) were added Pd(PPh₃)₂Cl₂ (44.29 mg, 0.05 mmol), CuI (20.66 mg, 0.11 mmol) and TEA (0.16 g, 1.63 mmol). The reaction mixture was degassed with argon for three times and stirred for 1.5 h at 90° C. The resulting mixture was concentrated under reduced pressure. The residue was purified by silica gel column chromatography, eluted with MeOH in DCM (0-4.8%). The fractions contained desired product were combined and concentrated. The crude product was further purified by Prep-HPLC with the following conditions Column: XBridge Prep C18 OBD Column, 19×150 mm, 5 μm; Mobile Phase A: Water (10 mM NH₄HCO₃), Mobile Phase B: ACN; Flow rate: 20 mL/min; Gradient: 30% B to 60% B in 4.3 min; Detector: UV 210 & 254 nm; RT: 4.02 min. The fractions contained desired product were combined and concentrated under reduced pressure to afford 3-[2-[1-(difluoromethyl)-4,6-difluoro-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide (44.8 mg, 15%) as a white solid. MS ESI calculated for C₂₄H₂₃F₄N₇O₃[M+H]⁺, 534.18, found 534.25. H-NMR (400 MHz, d₆-DMSO) δ 8.79 (s, 1H), 8.11 (t, J=58.4 Hz, 1H), 7.75 (d, J=8.4 Hz, 1H), 7.56 (brs, 1H), 6.71-6.55 (m, 3H), 6.17 (d, J=16.9 Hz, 1H), 5.69 (d, J=10.3 Hz, 1H), 5.28 (s, 1H), 4.53-4.39 (m, 1H), 4.02-3.72 (m, 2H), 3.60-3.45 (m, 2H), 3.32 (s, 3H), 2.96 (s, 3H), 2.67-2.61 (m, 1H), 2.33-2.30 (m, 1H).

### Example 31: 1-((3S,5R)-1-acryloyl-5-(methoxymethyl)pyrrolidin-3-yl)-3-((1-(difluoromethyl)-4,6-difluoro-2-methyl-1H-benzo[d]imidazol-5-yl)ethynyl)-5-(methylamino)-1H-pyrazole-4-carboxamide

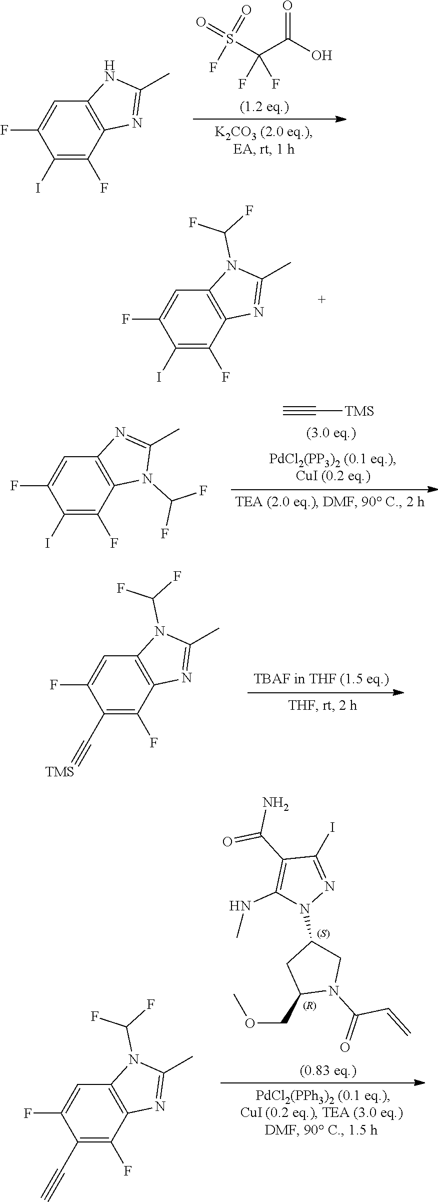

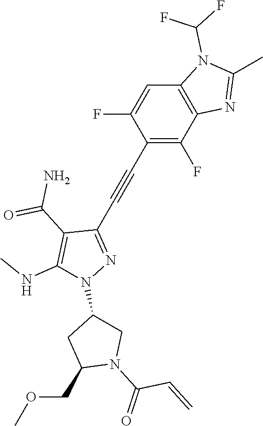

### Step 1: 1-(difluoromethyl)-4,6-difluoro-5-iodo-2-methyl-1,3-benzodiazole

To a stirred solution of 4,6-difluoro-5-iodo-2-methyl-1H-1,3-benzodiazole (0.20 g, 0.68 mmol) and K₂CO₃ (0.19 g, 1.36 mmol) in EA (3.00 mL) was added difluoro(sulfo)acetic acid (0.15 g, 0.82 mmol) dropwise at 0° C. The reaction mixture was stirred for 1 h at ambient temperature. The resulting mixture was diluted with water (10 mL) and extracted with EA (3×10 mL). The combined organic layers were washed with brine (2×10 mL), dried over anhydrous Na₂SO₄ and filtered. The filtrate was concentrated under reduced pressure. The residue was purified by silica gel column chromatography, eluted with EA in PE (10-35%). The fractions contained desired product were combined and concentrated to afford 1-(difluoromethyl)-4,6-difluoro-5-iodo-2-methyl-1,3-benzodiazole (80 mg, 34%) as a light yellow solid. MS ESI calculated for C₉H₅F₄IN₂ [M+H]⁺, 344.95, found 345.00.

### Step 2: 1-(difluoromethyl)-4,6-difluoro-2-methyl-5-[2-(trimethylsilyl)ethynyl]-1,3-benzodiazole

To a stirred mixture of 1-(difluoromethyl)-4,6-difluoro-5-iodo-2-methyl-1,3-benzodiazole (0.61 g, 1.77 mmol), trimethylsilylacetylene (0.52 g 5.32 mmol), PdCl₂(PPh₃)₂ (0.12 g, 0.18 mmol) and CuI (67.53 mg, 0.36 mmol) in DMF (8.00 mL) was added TEA (0.36 g, 3.55 mmol). The reaction mixture was degassed with argon for three times and stirred for 2 h at 90° C. After cooling down to ambient temperature, the resulting mixture was diluted with water (20 mL) and extracted with EA (3×25 mL). The combined organic layers were washed with brine (2×30 mL), dried over anhydrous Na₂SO₄ and filtered. The filtrate was concentrated under reduced pressure. The residue was purified by silica gel column chromatography, eluted of EA in PE (20-50%). The fractions contained desired product were combined and concentrated under reduced pressure to afford 1-(difluoromethyl)-4,6-difluoro-2-methyl-5-[2-(trimethylsilyl)ethynyl]-1,3-benzodiazole (0.5 g, 89%) as a yellow solid. MS ESI calculated for C₁₄H₁₄F₄N₂Si [M+H]⁺, 315.09, found 315.15.

### Step 3: 1-(difluoromethyl)-5-ethynyl-4,6-difluoro-2-methyl-1,3-benzodiazole

To a stirred solution of 1-(difluoromethyl)-4,6-difluoro-2-methyl-5-[2-(trimethylsilyl)ethynyl]-1,3-benzodiazole (0.50 g, 1.59 mmol) in THF (5.00 mL) was added TBAF (1.0 M in THF, 2.40 mL, 2.40 mmol) dropwise at 0° C. The reaction mixture was stirred for 2 h at ambient temperature under argon atmosphere. The resulting mixture was diluted with water (20 mL) and extracted with EA (3×20 mL). The combined organic layers were washed with brine (30 mL), dried over anhydrous Na₂SO₄ and filtered. The filtrate was concentrated under reduced pressure. The residue was purified by silica gel column chromatography, eluted with EA in PE (50-90%). The fractions contained desired product were combined and concentrated under reduced pressure to afford 1-(difluoromethyl)-5-ethynyl-4,6-difluoro-2-methyl-1,3-benzodiazole (0.33 g, 85%) as a yellow solid. MS ESI calculated for C₁₁H₆F₄N₂[M+H]⁺, 243.05, found 242.85.

### Step 4: 3-[2-[1-(difluoromethyl)-4,6-difluoro-2-methyl-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide

To a mixture of 3-iodo-1-[(3S,5R)-5-(methoxymethyl)-1-(prop-2-enoyl)pyrrolidin-3-yl]-5-(methylamino)pyrazole-4-carboxamide (0.13 g, 0.30 mmol), 1-(difluoromethyl)-5-ethynyl-4,6-difluoro-2-methyl-1,3-benzodiazole (87.20 mg, 0.36 mmol), PdCl₂(PPh₃)₂ (21.06 mg, 0.03 mmol) and CuI (11.43 mg, 0.06 mmol) in DMF (2.00 mL) was added TEA (60.73 mg, 0.60 mmol). The reaction mixture was degassed with argon for three times and stirred for 1.5 h at 90° C. After cooling down to ambient temperature, the resulting mixture was concentrated under reduced pressure. The residue was purified by silica gel column chromatography, eluting with MeOH in DCM (0-5.0%) to afford crude product. The crude product was further purified by Prep-HPLC with the following conditions Column: XBridge Prep C18 OBD Column, 19×150 mm 5 um; Mobile Phase A: Water (10 mM NH₄HCO₃), Mobile Phase B: ACN; Flow rate: 20 mL/min; Gradient: 25% B to 50% B in 4.3 min; Detector: UV 210 & 254 nm; RT: 4.02 min. The fractions contained desired product were combined and concentrated under reduced pressure to afford 3-[2-[1-(difluoromethyl)-4,6-difluoro-2-methyl-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide (86 mg, 52%) as a white solid. MS ESI calculated for C₂₅H₂₅F₄N₇O₃[M+H]⁺, 548.20, found 548.05. H NMR (400 MHz, d₆-DMSO) δ 8.11 (t, J=57.2 Hz, 1H), 7.67 (d, J=8.8 Hz, 1H), 7.56 (brs, 1H), 6.75-6.56 (m, 3H), 6.18-6.15 (m, 1H), 5.71-5.68 (m, 1H), 5.32-5.20 (m, 1H), 4.55-4.36 (m, 1H), 4.05-3.71 (m, 2H), 3.62-3.44 (m, 2H), 3.30 (d, J=5.7 Hz, 3H), 2.96 (t, J=5.5 Hz, 3H), 2.69 (s, 3H), 2.67-2.46 (m, 1H), 2.34-2.27 (m, 1H).

### Example 32: 1-((3S,5R)-1-acryloyl-5-(methoxymethyl)pyrrolidin-3-yl)-3-((1-ethyl-6-fluoro-1H-benzo[d]imidazol-5-yl)ethynyl)-5-(methylamino)-1H-pyrazole-4-carboxamide

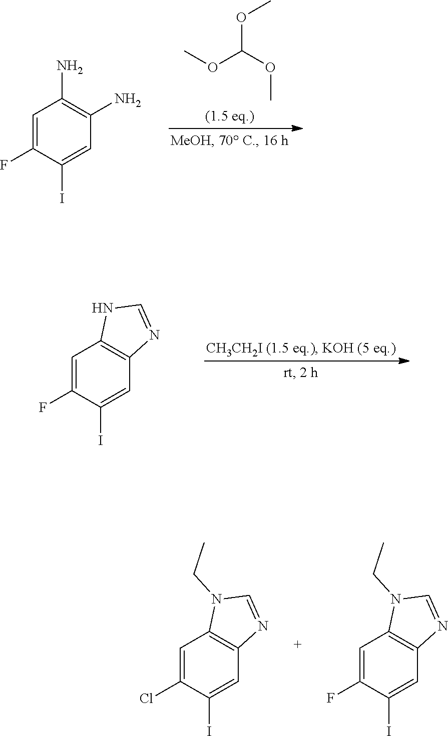

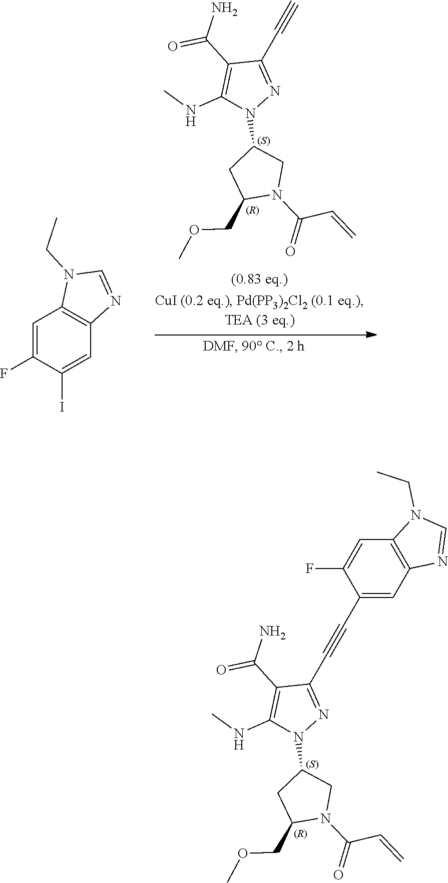

### Step 1: 5-fluoro-6-iodo-3H-1,3-benzodiazole

To a solution of 4-fluoro-5-iodobenzene-1,2-diamine (3.17 g, 12.58 mmol) in MeOH (30 mL) was added trimethyl orthoformate (2.00 g, 18.85 mmol). The reaction mixture was stirred for overnight at 70° C. under nitrogen atmosphere. The resulting mixture was concentrated under reduced pressure. The residue was purified by silica gel column chromatography, eluted with CH₂Cl₂/MeOH (10:1). The fractions contained desired product were combined and concentrated to afford 5-fluoro-6-iodo-3H-1,3-benzodiazole (2.9 g, 88%) as a light brown solid. MS ESI calculated for C₇H₄FIN₂ [M+H]⁺, 262.94; found 262.95.

### Step 2: 1-ethyl-6-fluoro-5-iodo-1,3-benzodiazole

To a stirred solution of 5-fluoro-6-iodo-3H-1,3-benzodiazole (1.10 g, 4.20 mmol) and KOH (1.18 g, 20.99 mmol) in acetone (16.50 mL) was added iodoethane (0.50 mL, 3.23 mmol) dropwise at 0° C. under nitrogen atmosphere. The reaction mixture was stirred for 2 h at room temperature. The resulting mixture was diluted with water (100 mL), extracted with EtOAc (3×100 mL). The combined organic layers were washed with brine (100 mL), dried over anhydrous Na₂SO₄ and filtered. The filtrate was concentrated under reduced pressure. The residue was purified by silica gel column chromatography, eluted with CH₂Cl₂/MeOH (15:1). The fractions contained desired product were combined and concentrated to afford 1.4 g crude product. The crude product was purified by Prep-Achiral-SFC with the following conditions Column: CHIRAL ART Cellulose-SB, 3*25 cm, 5 um; Mobile Phase A: CO₂, Mobile Phase B: MeOH (0.1% 2M NH₃-MeOH); Flow rate: 100 mL/min; Gradient: 20% B; 220 nm; RT1: 3.7; RT2: 4.3; Injection Volume: 2 ml; Number Of Runs: 20). The fractions contained desired product (last peak RT2: 4.3) were combined and concentrated to afford 1-ethyl-6-fluoro-5-iodo-1,3-benzodiazole (0.44 g, 32%) as a light brown solid.

### Step 3: 3-[2-(1-ethyl-6-fluoro-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a stirred mixture of 3-ethynyl-1-[(3S,5R)-5-(methoxymethyl)-1-(prop-2-enoyl)pyrrolidin-3-yl]-5-(methylamino)pyrazole-4-carboxamide (0.18 g, 0.54 mmol), 1-ethyl-6-fluoro-5-iodo-1,3-benzodiazole (0.19 g, 0.65 mmol), Pd(PPh₃)₂Cl₂ (38.13 mg, 0.05 mmol) and CuI (20.69 mg, 0.11 mmol) in DMF (2.00 mL) was added TEA (0.23 mL, 2.24 mmol). The reaction mixture was degassed with argon for three times and stirred for 2 h at 90° C. The resulting mixture was concentrated under reduced pressure. The residue was purified by silica gel column chromatography, eluted with 6% MeOH in DCM. The fractions contained desired product were combined and concentrated. The crude product (200 mg) was purified by Prep-HPLC with the following conditions Column: XBridge Prep C18 OBD Column, 19×150 mm 5 um; Mobile Phase A: Water (10 mmol/L NH₄HCO₃), Mobile Phase B: ACN; Flow rate: 20 mL/min; Gradient: 25 B to 55 B in 4.3 min; 210/254 nm; RT1: 4.02. The fractions contained desired product were combined and concentrated to afford 3-[2-(1-ethyl-6-fluoro-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (62.9 mg, 23%) as an off-white solid. MS ESI calculated for C₂₅H₂₈FN₇O₃[M+H]⁺, 494.22, found 494.25. H-NMR (400 MHz, DMSO-d₆): δ 8.41 (s, 1H), 7.95 (d, J=6.2 Hz, 1H), 7.76 (d, J=9.7 Hz, 1H), 7.49 (s, 1H), 6.87-6.49 (m, 3H), 6.19-6.17 (m, 1H), 5.75-5.65 (m, 1H), 5.29-5.25 (m, 1H), 4.50-4.45 (m, 1H), 4.28 (q, J=7.3 Hz, 2H), 4.07-3.44 (m, 3H), 3.35-3.29 (m, 3H), 2.96 (t, J=5.2 Hz, 3H), 2.47 (t, J=7.1 Hz, 1H), 2.45-2.44 (m, 1H), 2.35-2.24 (m, 1H), 1.41 (t, J=7.3 Hz, 3H).

### Example 33: 1-((3S,5R)-1-acryloyl-5-(methoxymethyl)pyrrolidin-3-yl)-3-((6-chloro-1-ethyl-2-methyl-1H-benzo[d]imidazol-5-yl)ethynyl)-5-(methylamino)-1H-pyrazole-4-carboxamide

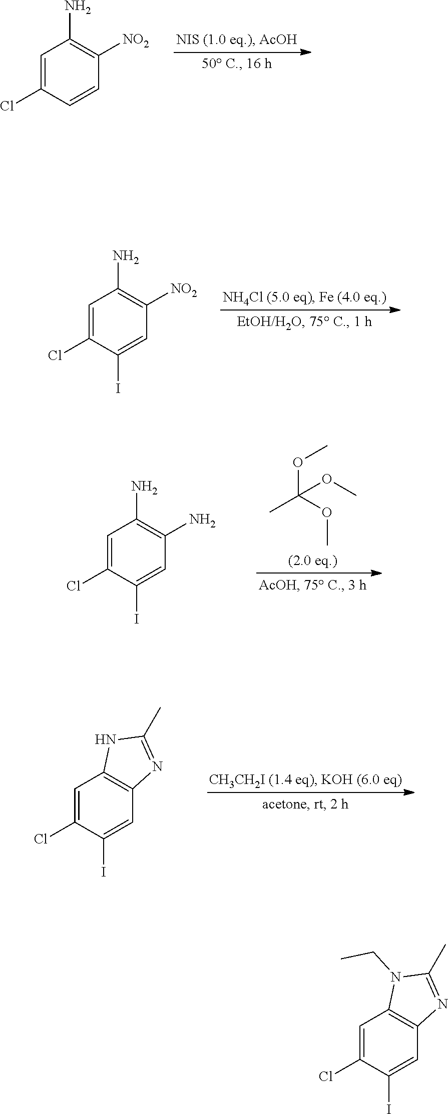

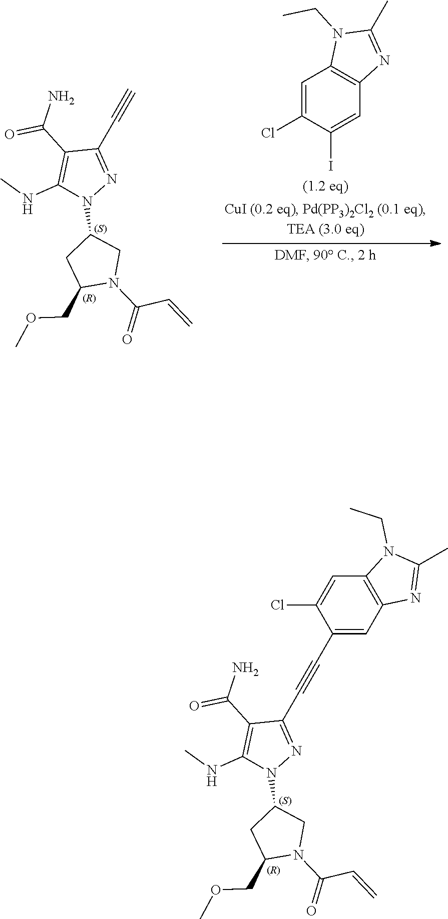

### Step 1: 5-chloro-4-iodo-2-nitroaniline

To a mixture of 5-chloro-2-nitroaniline (5.00 g, 28.97 mmol) in AcOH (50.00 mL) was added NIS (6.52 g, 28.98 mmol). The reaction mixture was stirred for overnight at 50° C. under argon atmosphere. The resulting mixture was concentrated under reduced pressure. The residue was quenched by the addition of sat. NaHCO₃ (aq.) (100 mL) at room temperature. The resulting mixture was extracted with EtOAc (2×300 mL). The combined organic layers were washed with water (2×150 mL), dried over anhydrous Na₂SO₄. and filtered. The filtrate was concentrated under reduced pressure. The residue was purified by silica gel column chromatography, eluted with PE/EtOAc (6:1). The fractions contained desired product were combined and concentrated to afford 5-chloro-4-iodo-2-nitroaniline (7.6 g, 87%) as a light yellow solid. H-NMR (400 MHz, DMSO-d₆) δ 8.36 (s, 1H), 7.60 (s, 2H), 7.27 (s, 1H).

### Step 2: 4-chloro-5-iodobenzene-1,2-diamine

To a mixture of Fe (5.61 g, 100.46 mmol) and 5-chloro-4-iodo-2-nitroaniline (7.50 g, 25.13 mmol) in EtOH (100.00 mL) and H₂O (25.00 mL) was added NH₄Cl (6.72 g, 125.63 mmol, 5.00 equiv). The reaction mixture was stirred for 1 h at 75° C. under argon atmosphere. The resulting mixture was filtered, the filter cake was washed with MeOH (3×100 mL). The filtrate was concentrated under reduced pressure. The resulting mixture was extracted with EA (2×200 mL). The combined organic layers were washed with EtOAc (2×200 mL), dried over anhydrous Na₂SO₄ and filtered. The filtrate was concentrated under reduced pressure to afford 4-chloro-5-iodobenzene-1,2-diamine (6.6 g, 97.83%) as a yellow solid which was used in the next step directly without further purification. MS ESI calculated for C₆H₆ClIN₂ [M+H]⁺, 268.93, found 268.85.

### Step 3: 5-chloro-6-iodo-2-methyl-3H-1,3-benzodiazole

To a solution of 4-chloro-5-iodobenzene-1,2-diamine (3.50 g, 13.04 mmol) in AcOH (40.00 mL) was added 1,1,1-trimethoxyethane (3.33 mL, 26.05 mmol). The reaction mixture was stirred for 3 h at 75° C. under argon atmosphere. The residue was neutralized to pH 7 with saturated NaHCO₃ (aq.). The resulting mixture was extracted with EtOAc (2×200 mL). The combined organic layers were washed with water (2×100 mL), dried over anhydrous Na₂SO₄ and filtered. The filtrate was concentrated under reduced pressure. The residue was purified by silica gel column chromatography, eluted with DCM/MeOH (20/1). The fractions contained desired product were combined and concentrated to afford 5-chloro-6-iodo-2-methyl-3H-1,3-benzodiazole (3.2 g, 83%) as an off-white solid. MS ESI calculated for C₈H₆ClIN₂ [M+H]⁺, 292.93, found 292.95.

### Step 4: 6-chloro-1-ethyl-5-iodo-2-methyl-1,3-benzodiazole

To a mixture of 5-chloro-6-iodo-2-methyl-3H-1,3-benzodiazole (3.20 g, 10.94 mmol) and KOH (3.68 g, 65.59 mmol) in acetone (40.00 mL) was added iodoethane (1.28 mL, 8.19 mmol). The reaction mixture was stirred for 2 h at room temperature under argon atmosphere. The resulting mixture was concentrated under reduced pressure. The residue was extracted with EtOAc (2×300 mL). The combined organic layers were washed with water (3×100 mL), dried over anhydrous Na₂SO₄ and filtered. The filtrate was concentrated under reduced pressure. The residue was purified by silica gel column chromatography, eluted with CH₂Cl₂/MeOH (10:1). The fractions contained desired product were combined and concentrated to afford 3.8 g of mixture. The crude product (2 g) was purified by Prep-SFC with the following conditions Column: CHIRALPAK IG, 5*25 cm, 10 um; Mobile Phase A: CO₂, Mobile Phase B: MeOH (0.1% 2M NH₃-MEOH); Flow rate: 200 mL/min; Gradient: 50% B; 220 nm; RT1: 5.99; RT2: 7.74; Injection Volume: 3 ml; Number Of Runs: 17. The fractions contained desired product were combined and concentrated to afford 6-chloro-1-ethyl-5-iodo-2-methyl-1,3-benzodiazole (0.80 g, 22%) as a light yellow solid. MS ESI calculated for C₁₀H₁₀ClIN₂ [M+H]⁺, 320.96, found 320.95.

### Step 5: 3-[2-(6-chloro-1-ethyl-2-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a mixture of 3-ethynyl-1-[(3S,5R)-5-(methoxymethyl)-1-(prop-2-enoyl)pyrrolidin-3-yl]-5-(methylamino)pyrazole-4-carboxamide (0.15 g, 0.45 mmol), 6-chloro-1-ethyl-5-iodo-2-methyl-1,3-benzodiazole (0.17 g, 0.54 mmol), CuI (17.24 mg, 0.09 mmol) and Pd(PPh₃)₂Cl₂ (31.77 mg, 0.05 mmol) in DMF (1.50 mL) was added TEA (0.19 mL, 1.87 mmol). The reaction mixture was degassed with argon for three times and stirred for 2 h at 90° C. The resulting mixture was concentrated under reduced pressure. The residue was purified by Prep-HPLC with the following conditions Column: XBridge Prep C18 OBD Column, 19×150 mm 5 um; Mobile Phase A: Water (10 mmol/L NH₄HCO₃), Mobile Phase B: ACN; Flow rate: 20 m/min; Gradient: 25 B to 50 B in 4.3 min; 210/254 nm; RT1:4.23. The fractions contained desired product were combined and concentrated to afford 3-[2-(6-chloro-1-ethyl-2-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (44.4 mg, 18.72%) as an off-white solid. MS ESI calculated for C₂₆H₃₀ClN₇O₃[M+H]⁺, 292.93, found 292.95. H-NMR (400 MHz, DMSO-4) S 7.92-7.84 (m, 2H), 7.51 (s, 1H), 6.86 (s, 1H), 6.77-6.55 (m, 2H), 6.22-6.13 (m, 1H), 5.73-5.65 (m, 1H), 5.33-5.21 (m, 1H), 4.58-4.36 (m, 1H), 4.30-4.20 (m, 2H), 4.08-3.70 (m, 2H), 3.65-3.42 (m, 2H), 3.33 (s, 1H), 3.31 (d, J=5.4 Hz, 3H), 3.00-2.93 (m, 3H), 2.56 (s, 3H), 2.31 (m, 1H), 1.29 (t, J=7.2 Hz, 3H).

### Example 34: 1-((3S,5R)-1-acryloyl-5-(methoxymethyl)pyrrolidin-3-yl)-3-((4,6-difluoro-1-(2,2,2-trifluoroethyl)-1H-benzo[d]imidazol-5-yl)ethynyl)-5-(methylamino)-1H-pyrazole-4-carboxamide

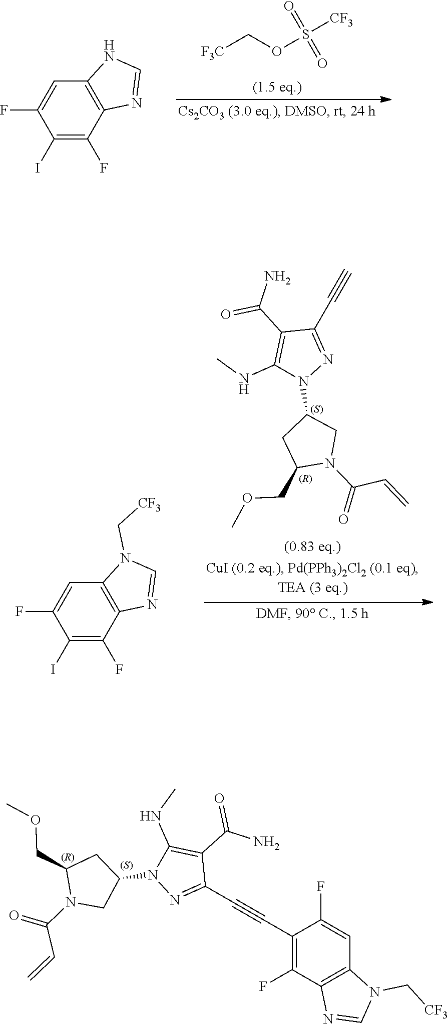

### Step 1: 4,6-difluoro-5-iodo-1-(2,2,2-trifluoroethyl)-1,3-benzodiazole

To a stirred mixture of 4,6-difluoro-5-iodo-1H-1,3-benzodiazole (0.80 g, 2.86 mmol) and trifluoro(trifluoromethanesulfonylmethoxy)methane (0.99 g, 4.28 mmol) in DMSO (8.00 mL) was added Cs₂CO₃ (2.79 g, 8.57 mmol) at room temperature. The reaction mixture was stirred for 24 h. The reaction was quenched by the addition of water (10 mL) at room temperature. The resulting mixture was extracted with EtOAc (3×20 mL). The combined organic layers were washed with brine (3×20 mL), dried over anhydrous Na₂SO₄ and filtered. The filtrate was concentrated under reduced pressure. The residue was purified by SFC to afford 4,6-difluoro-5-iodo-1-(2,2,2-trifluoroethyl)-1,3-benzodiazole (0.32 g, 30%) as a light yellow solid. MS ESI calculated for C₉H₄F₅IN₂ [M+H]⁺, 363.04, found 363.00.

### Step 2: 3-[2-[4,6-difluoro-1-(2,2,2-trifluoroethyl)-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide

To a stirred mixture of 3-ethynyl-1-[(3S,5R)-5-(methoxymethyl)-1-(prop-2-enoyl) pyrrolidin-3-yl]-5-(methylamino) pyrazole-4-carboxamide (0.15 g, 0.45 mmol), 4,6-difluoro-5-iodo-1-(2,2,2-trifluoroethyl)-1,3-benzodiazole (0.20 g, 0.54 mmol), Pd(PPh₃)₂Cl₂ (31.77 mg, 0.05 mmol) and CuI (17.24 mg, 0.09 mmol) in DMF (1.50 mL) were added TEA (0.14 g, 1.36 mmol). The reaction mixture was degassed with argon for three times and stirred for 1.5 h at 90° C. The resulting mixture was concentrated under reduced pressure. The residue was purified by Prep-HPLC Column: XBridge Prep C18 OBD Column, 19×150 mm 5 um; Mobile Phase A: Water (10 mmol/L NH₄HCO₃), Mobile Phase B: ACN; Flow rate: 20 mL/min; Gradient: 25 B to 55 B in 4.3 min; 210/254 nm; RT1: 4.12. The fractions contained desired product were combined and concentrated to afford 3-[2-[4,6-difluoro-1-(2,2,2-trifluoroethyl)-1,3-benzodiazol-5-yl]ethynyl]-1-[(3S,5R)-5-(methoxymethyl)-1-(prop-2-enoyl) pyrrolidin-3-yl]-5-(methylamino) pyrazole-4-carboxamide (0.10 g, 39%) as an off-white solid. MS ESI calculated for C₂₅H₂₄F₅N₇O₃[M+H]⁺, 566.19, found 566.20. ¹H NMR (400 MHz, d₆-DMSO) δ 8.51 (s, 1H), 7.81 (d, J=9.1 Hz, 1H), 7.56 (s, 1H), 6.75-6.56 (m, 3H), 6.17 (d, J=16.6 Hz, 1H), 5.69 (d, J=9.5 Hz, 1H), 5.43-5.27 (m, 3H), 4.57-4.37 (m, 1H), 4.08-3.83 (m, 2H), 3.79-3.53 (m, 2H), 3.52-3.34 (m, 3H), 2.98-2.94 (m, 3H), 2.51 (m, 1H), 2.33-2.29 (m, 1H).

### Example 35: 1-((3S,5R)-1-acryloyl-5-(methoxymethyl)pyrrolidin-3-yl)-3-((4,6-difluoro-2-methyl-1-(2,2,2-trifluoroethyl)-1H-benzo[d]imidazol-5-yl)ethynyl)-5-(methylamino)-1H-pyrazole-4-carboxamide

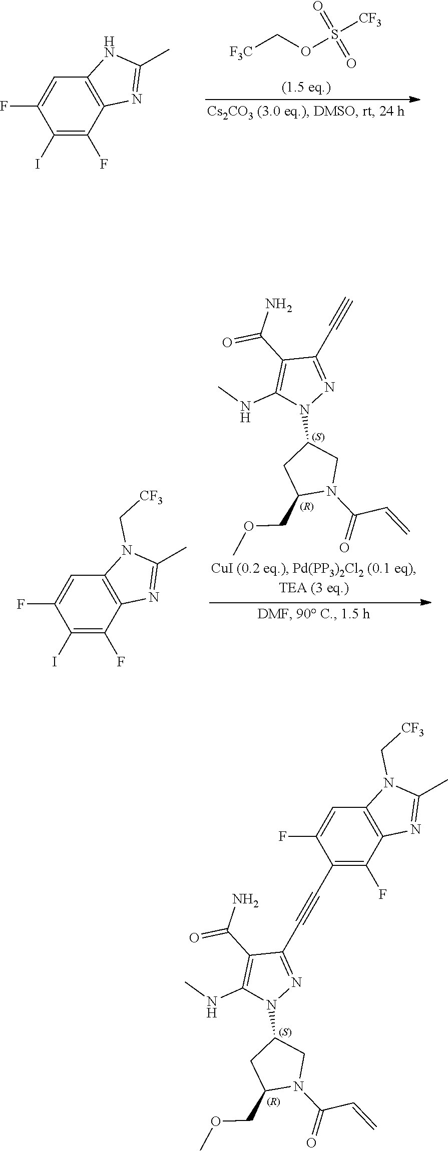

### Step 1: Tert-butyl 2-(1,1-difluoroethyl)morpholine-4-carboxylate

To a stirred solution of 4,6-difluoro-5-iodo-2-methyl-1H-1,3-benzodiazole (1.00 g, 3.40 mmol) in DMSO (10.00 mL) was added trifluoro(trifluoromethanesulfonylmethoxy)methane (1.18 g, 5.10 mmol) and Cs₂CO₃ (3.32 g, 0.01 mmol) in portions at room temperature. The reaction mixture was stirred for 24 h at room temperature. The reaction was quenched by the addition of water (10 mL) at room temperature. The resulting mixture was extracted with EtOAc (3×20 mL). The combined organic layers were washed with brine (3×20 mL), dried over anhydrous Na₂SO₄ and filtered. The filtrate was concentrated under reduced pressure. The residue was purified by SFC Column: DAICEL DCpak P4VP (02), 30*250 mm, 5 um; Mobile Phase A: CO₂, Mobile Phase B: MeOH (0.1% 2M NH₃-MeOH); Flow rate: 60 mL/min; Gradient: 18% B; 254 nm; RT2: 6.21; Injection Volume: 0.8 ml; Number of Runs: 15. The fractions contained desired product were combined and concentrated to afford 4,6-difluoro-5-iodo-2-methyl-1-(2,2,2-trifluoroethyl)-1,3-benzodiazole (0.42 g, 32%) as a light yellow solid. MS ESI calculated for C₁₀H₆F₅IN₂ [M+H]⁺, 376.95, found 377.00.

### Step 2: 3-[2-[4,6-Difluoro-2-methyl-1-(2,2,2-trifluoroethyl)-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide

To a stirred mixture of 3-ethynyl-1-[(3S,5R)-5-(methoxymethyl)-1-(prop-2-enoyl)pyrrolidin-3-yl]-5-(methylamino)pyrazole-4-carboxamide (0.15 g, 0.45 mmol), 4,6-difluoro-5-iodo-2-methyl-1-(2,2,2-trifluoroethyl)-1,3-benzodiazole (0.20 g, 0.54 mmol), Pd(PPh₃)₂Cl₂ (31.77 mg, 0.05 mmol) and CuI (17.24 mg, 0.09 mmol) in DMF (1.50 mL) was added TEA (0.14 g, 1.36 mmol) at room temperature. The reaction mixture was degassed with argon for three times and stirred for 1.5 h at 90° C. The resulting mixture was concentrated under reduced pressure. The residue was purified by Prep-HPLC Column: XBridge Prep C18 OBD Column, 19×150 mm 5 um; Mobile Phase A: Water (10 mmol/L NH₄HCO₃), Mobile Phase B: ACN; Flow rate: 20 mL/min; Gradient: 25 B to 60 B in 4.3 min; 210/254 nm; RT1: 4.02. The fractions contained desired product were combined and concentrated to afford 3-[2-[4,6-difluoro-2-methyl-1-(2,2,2-trifluoroethyl)-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide (0.10 g, 37%) as an off-white solid. MS ESI calculated for C₂₆H₂₆F₅N₇O₃[M+H]⁺, 580.20, found 580.25. ¹H NMR (400 MHz, DMSO-d₆) δ 7.70 (d, J=9.1 Hz, 1H), 7.55 (s, 1H), 6.86-6.49 (m, 3H), 6.19-6.14 (m, 1H), 5.71-5.67 (m, 1H), 5.42-5.19 (m, 3H), 4.62-4.35 (m, 1H), 4.08-3.70 (m, 2H), 3.65-3.42 (m, 2H), 3.33-3.27 (m, 3H), 2.96 (t, J=5.5 Hz, 3H), 2.60 (s, 3H), 2.52-2.48 (m, 1H), 2.37-2.25 (m, 1H).

### Example 36: 3-[2-(3-ethyl-4-fluoro-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

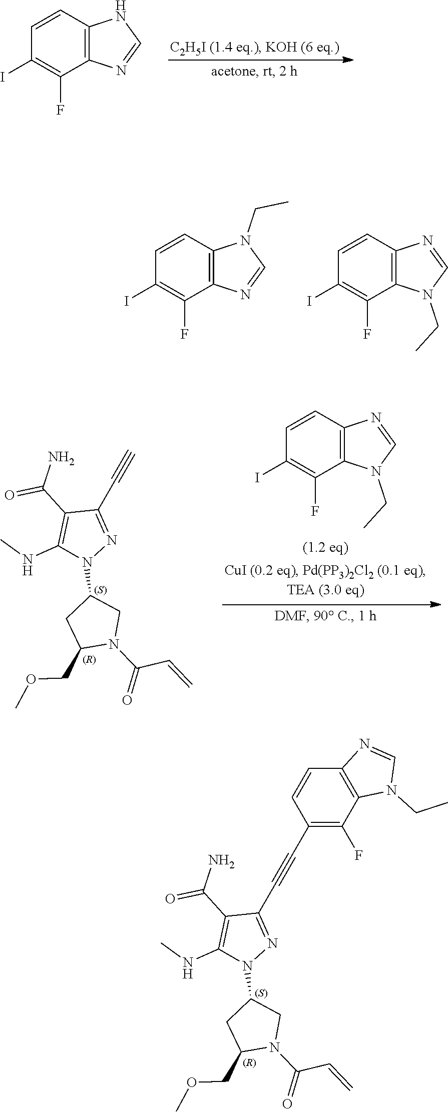

### Step 1: 1-ethyl-4-fluoro-5-iodo-1,3-benzodiazole and 1-ethyl-7-fluoro-6-iodo-1,3-benzodiazole

To a stirred mixture of 4-fluoro-5-iodo-1H-1,3-benzodiazole (2.00 g, 7.63 mmol) and KOH (2.57 g, 45.80 mmol) in acetone (20.00 mL) was added iodoethane (1.67 g, 10.69 mmol) at room temperature under air atmosphere. The reaction mixture was stirred for 2 h at room temperature. The resulting mixture was diluted with water (50 mL) and extracted with EA (3×70 mL). The combined organic layers were washed with brine (2×50 mL), dried over anhydrous Na₂SO₄ and filtered. The filtrate was concentrated under reduced pressure. The residue was purified by reverse flash chromatography with the following conditions: column, C18 silica gel; mobile phase, ACN in water (1 g/NH₄HCO₃), 25% to 55% gradient in 30 min; detector, UV 254 nm. The fraction contained desired product were combined and concentrated to afford 1-ethyl-4-fluoro-5-iodo-1,3-benzodiazole (0.80 g, 36%) as a yellow solid and 1-ethyl-7-fluoro-6-iodo-1,3-benzodiazole (0.78 g, 35%) as a yellow solid. PH-FNT-KIN-03-1434-1: MS ESI calculated for C₉H₈FIN₂ [M+H]⁺, 290.97, found 290.99. PH-FNT-KIN-03-1300-1: MS ESI calculated for C₉H₈FIN₂ [M+H]⁺, 290.97, found 290.95.

### Step 2: 3-[2-(3-ethyl-4-fluoro-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a stirred mixture of 3-ethynyl-1-[(3S,5R)-5-(methoxymethyl)-1-(prop-2-enoyl)pyrrolidin-3-yl]-5-(methylamino)pyrazole-4-carboxamide (0.18 g, 0.54 mmol), 1-ethyl-7-fluoro-6-iodo-1,3-benzodiazole (0.19 g, 0.65 mmol), CuI (20.69 mg, 0.11 mmol) and Pd(pph₃)Cl₂ (44.36 mg, 0.05 mmol) in DMF (2.00 mL) was added TEA (0.23 mL, 2.24 mmol). The reaction mixture was degassed with argon for three times and stirred for 1 h at 90° C. The resulting mixture was concentrated under reduced pressure. The residue was purified by Prep-HPLC with the following conditions Column: XBridge Prep OBD C18 Column, 19*250 mm, 5 um; Mobile Phase A: Water (10 mmol/L NH₄HCO₃), Mobile Phase B: ACN; Flow rate: 20 mL/min; Gradient: 25 B to 60 B in 5.8 min; 210/254 nm; RT1: 5.57. The fractions contained desired product were combined and concentrated to afford 3-[2-(3-ethyl-4-fluoro-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (85 mg, 31%) as an off-white solid. MS ESI calculated for C₂₅H₂₈FN₇O₃[M+H]⁺, 494.22, found 494.25. H-NMR (400 MHz, DMSO-d₆) δ 8.42 (s, 1H), 7.64-7.29 (m, 3H), 6.93-6.46 (m, 3H), 6.17 (d, J=16.4 Hz, 1H), 5.74-5.65 (m, 1H), 5.33-5.20 (m, 1H), 4.62-4.30 (m, 3H), 4.07-3.71 (m, 2H), 3.65-3.41 (m, 2H), 3.32-3.28 (m, 1H), 3.03-2.92 (m, 3H), 2.51 (s, 3H), 2.35-2.30 (m, 1H), 1.47-1.44 (m, 3H).

### Example 37: 3-[2-(1-ethyl-6-fluoro-2-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

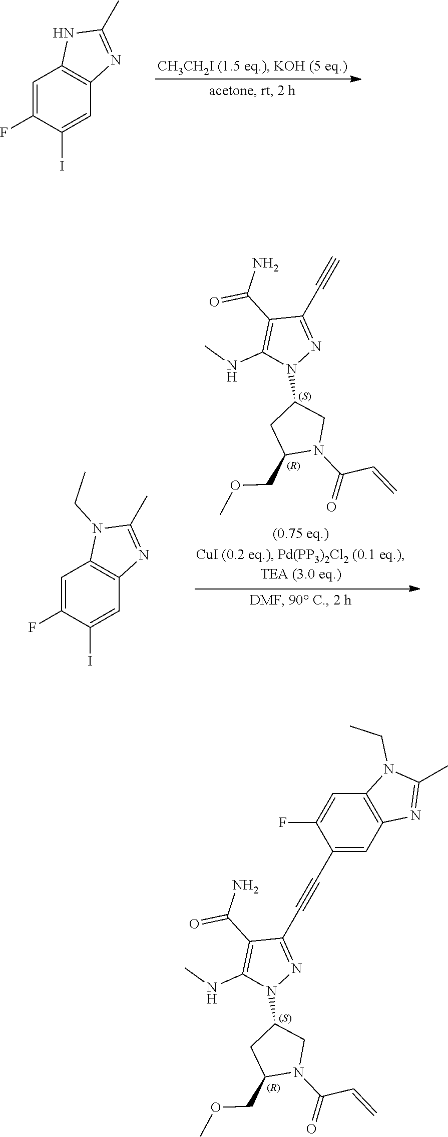

### Step 1: 1-ethyl-6-fluoro-5-iodo-2-methyl-1,3-benzodiazole

To a stirred solution of 5-fluoro-6-iodo-2-methyl-3H-1,3-benzodiazole (1.1 g, 3.98 mmol) and KOH (1.12 g, 19.92 mmol) in acetone (16.50 mL) was added iodoethane (0.48 mL, 3.07 mmol) dropwise at 0° C. under nitrogen atmosphere. The reaction mixture was stirred for 2 h at room temperature. The resulting mixture was diluted with water (100 mL), extracted with EtOAc (3×100 mL). The combined organic layers were washed with brine (100 mL), dried over anhydrous Na₂SO₄ and filtered. The filtrate was concentrated under reduced pressure. The residue was purified by silica gel column chromatography, eluted with CH₂Cl₂/MeOH (15:1). The fractions contained desired product were combined and concentrated to afford 1.29 g crude product. The crude product was purified by Prep-Achiral-SFC with the following conditions Column: CHIRAL ART Cellulose-SB, 3*25 cm, 5 um; Mobile Phase A: CO₂, Mobile Phase B: MeOH (0.1% 2M NH₃-MeOH); Flow rate: 100 mL/min; Gradient: 20% B; 220 nm; RT1: 3.7; RT2: 4.3; Injection Volume: 2 ml; Number Of Runs: 20. The fractions contained desired product (slower peak RT2:4.3) were combined and concentrated to afford 1-ethyl-6-fluoro-5-iodo-2-methyl-1,3-benzodiazole (0.48 g, 35%) as a light brown solid. ¹H NMR (300 MHz, Chloroform-d) δ 8.02 (d, J=5.6 Hz, 1H), 7.05 (d, J=7.8 Hz, 1H), 4.11 (q, J=7.3 Hz, 2H), 2.60 (s, 3H), 1.40 (t, J=7.3 Hz, 3H)

### Step 2: 3-[2-(1-ethyl-6-fluoro-2-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a stirred solution of 3-ethynyl-1-[(3S,5R)-5-(methoxymethyl)-1-(prop-2-enoyl)pyrrolidin-3-yl]-5-(methylamino)pyrazole-4-carboxamide (0.10 g, 0.31 mmol), 1-ethyl-6-fluoro-5-iodo-2-methyl-1,3-benzodiazole (0.14 g, 0.45 mmol), Pd(PPh₃)₂Cl₂ (21.18 mg, 0.03 mmol) and CuI (11.49 mg, 0.06 mmol) in DMF (1.00 mL) was added TEA (91.61 mg, 0.91 mmol). The reaction mixture was degassed with argon for three times and stirred for 2 h at 90° C. The resulting mixture was purified by reverse flash chromatography with the following conditions: column, C18 silica gel; mobile phase ACN, NH₄HCO₃ 0.01 mmol in water, 25% to 40% gradient in 20 min; detector, UV 254 nm. The fractions contained desired product were combined and concentrated. The crude product was purified by Prep-HPLC with the following conditions Column: Atlantis Prep T3 OBD Column, 19*250 mm 10 u; Mobile Phase A: Water (0.1% FA), Mobile Phase B: MeOH—HPLC; Flow rate: 20 mL/min; Gradient: 15 B to 50 B in 6 min; 210/254 nm; RT1: 5.56. The fractions contained desired product were combined and concentrated to afford 3-[2-(1-ethyl-6-fluoro-2-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (32.8 mg, 21%) as a light yellow solid. ESI calculated for C₂₆H₃₀FN₇O₃[M+H], 508.23; found 508.25. ¹H NMR (300 MHz, DMSO-d₆) δ 7.78 (d, J=6.3 Hz, 1H), 7.64 (d, J=9.9 Hz, 1H), 7.48 (d, J=8.0 Hz, 1H), 6.93-6.51 (m, 3H), 6.16-5.88 (m, 1H), 5.68-5.42 (m, 1H), 5.25-5.01 (m, 1H), 4.47-4.43 (m, 1H), 4.21 (q, J=7.2 Hz, 2H), 4.10-3.73 (m, 2H), 3.64-3.43 (m, 2H), 3.37-3.26 (m, 3H), 2.95 (d, J=4.3 Hz, 3H), 2.54 (s, 4H), 2.33-2.28 (m, 1H), 1.30-1.25 (m, 3H).

### Example 38: 3-[2-(4-chloro-1-ethyl-2-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

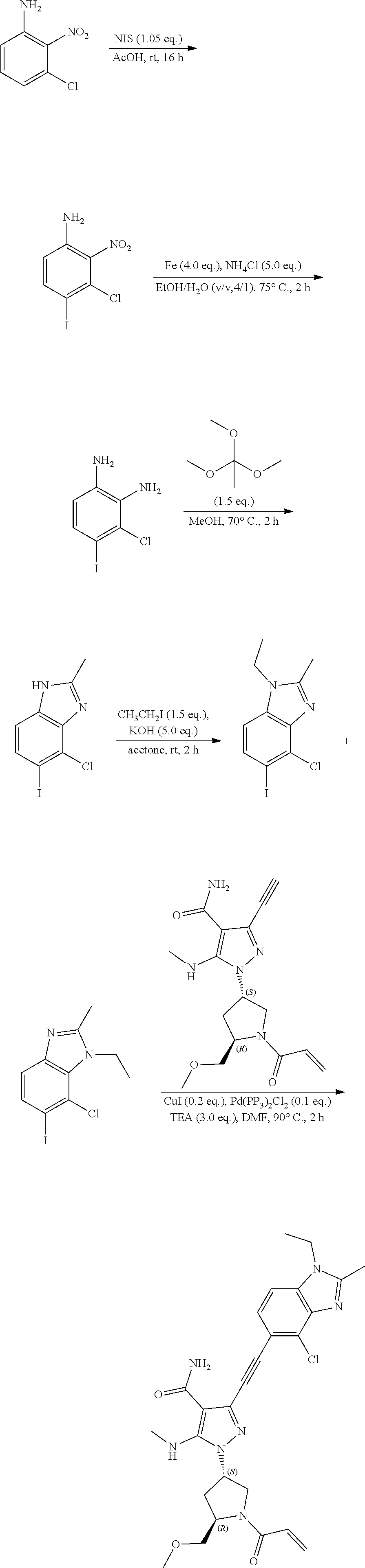

### Step 1: 3-chloro-4-iodo-2-nitroaniline

To a stirred solution of 3-chloro-2-nitroaniline (5.00 g, 28.97 mmol) in AcOH (50.00 mL) was added NIS (6.84 g, 30.40 mmol) at room temperature. The resulting reaction was stirred for 16 h at room temperature. The resulting mixture was concentrated under reduced pressure. The residue was dissolved in water (50 mL). The mixture was extracted with EA (3×100 mL) and the combined organic layers was dried over anhydrous Na₂SO₄. After filtration, the filtrate was concentrated under reduced pressure to afford 3-chloro-4-iodo-2-nitroaniline (8.50 g, 98%) as an orange solid which was used in the next step directly without further purification. MS ESI calculated for C₆H₄ClIN₂O₂[M−H]⁻, 296.89, 297.90; found 297.00, 299.00.

### Step 2: 3-chloro-4-iodobenzene-1,2-diamine

To a stirred mixture of 3-chloro-4-iodo-2-nitroaniline (1.00 g, 3.35 mmol) in EtOH (12.00 mL) and H₂O (3.00 mL) were added Fe (0.75 g, 13.43 mmol) and NH₄Cl (0.90 g, 16.75 mmol) at room temperature. The reaction mixture was stirred for 2 h at 75° C. The resulting mixture was allowed to cool down to room temperature. The resulting mixture was filtered, the filter cake was washed with EtOH (3×10 mL). The filtrate was concentrated under reduced pressure. The residue was dissolved in water (20 mL), extracted with EA (3×50 mL). The combined organic layers were washed with brine (3×10 mL), dried over anhydrous Na₂SO₄ and filtered. The filtrate was concentrated under reduced pressure to afford 3-chloro-4-iodobenzene-1,2-diamine (0.79 g, 87%) as a brown solid which was used in the next step directly without further purification. MS ESI calculated for C₆H₆ClIN₂[M+H]⁺, 268.93, 270.93; found 268.85, 270.85.

### Step 3: 3-chloro-4-iodobenzene-1,2-diamine

To a stirred solution of 3-chloro-4-iodobenzene-1,2-diamine (0.79 g 2.94 mmol) in MeOH (10.00 mL) was added 1,1,1-trimethoxyethane (0.53 g, 4.41 mmol) at room temperature. The reaction mixture was stirred for 2 h at 70° C. The resulting mixture was allowed to cool down to room temperature. The residue was purified by silica gel column chromatography, eluted with EA in PE (0-70%). The fractions contained desired product were combined and concentrated to afford 4-chloro-5-iodo-2-methyl-1H-1,3-benzodiazole (0.67 g, 77%) as a brown solid. MS ESI calculated for C₈H₆ClIN₂ [M+H]⁺, 292.93, 294.93; found 292.80, 294.80.

### Step 4: 4-chloro-1-ethyl-5-iodo-2-methyl-1,3-benzodiazole

To a stirred solution of 4-chloro-5-iodo-2-methyl-1H-1,3-benzodiazole (0.52 g 1.77 mmol) in acetone (6.00 mL) were added ethyl iodide (0.42 g, 2.66 mmol) and KOH (0.50 g, 8.88 mmol) at room temperature. The reaction mixture was stirred for 2 h at room temperature. The resulting mixture was concentrated under reduced pressure. The residue was purified by silica gel column chromatography, eluted with 3% MeOH in DCM. The fractions contained desired product were combined and concentrated to afford 4-chloro-1-ethyl-5-iodo-2-methyl-1,3-benzodiazole (0.23 g, 40%) as an off-white solid. Also eluted 4% MeOH in DCM. The fractions contained desired product were combined and concentrated to afford 7-chloro-1-ethyl-6-iodo-2-methyl-1,3-benzodiazole (0.11 mg, 19%) as a yellow solid. MS ESI calculated for C₁₀H₁₀ClIN₂ [M+H]⁺, 320.96, 322.96; found 320.85, 322.85.

### Step 5: 3-[2-(4-chloro-1-ethyl-2-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a stirred mixture of 3-ethynyl-1-[(3S,5R)-5-(methoxymethyl)-1-(prop-2-enoyl)pyrrolidin-3-yl]-5-(methylamino)pyrazole-4-carboxamide (0.13 g, 0.39 mmol) and 4-chloro-1-ethyl-5-iodo-2-methyl-1,3-benzodiazole (0.15 g, 0.47 mmol) in DMF (3.00 mL) were added Pd(PPh₃)₂Cl₂ (27.54 mg, 0.03 mmol), CuI (14.94 mg, 0.07 mmol) and TEA (0.12 g, 1.17 mmol). The reaction mixture was degassed with argon for three times and stirred for 2 h at 90° C. The resulting mixture was concentrated under reduced pressure. The residue was purified by silica gel column chromatography, eluted with MeOH in DCM (0-6.0%). The fractions contained desired product were combined and concentrated. The crude product (0.15 g) was purified by Prep-HPLC with the following conditions Column: SunFire Prep C18 OBD Column, 19×150 mm 5 um 10 nm; Mobile Phase A: Water (0.1% FA), Mobile Phase B: ACN; Flow rate: 20 mL/min; Gradient: 20 B to 50 B in 4.3 min; 210/254 nm; RT1: 4.02 min. The fractions contained desired product were combined and concentrated to afford 3-[2-(4-chloro-1-ethyl-2-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (49.30 mg, 23%) as an off-white solid. MS ESI calculated for C₂₆H₃₀ClN₇O₃[M+H]⁺, 524.22, 526.21; found 524.15. 526.15. H-NMR (300 MHz, DMSO-d₆) δ 7.81-7.41 (m, 3H), 6.88 (s, 1H), 6.65 (d, J=12.3 Hz, 1H), 6.57 (d, J=12.8 Hz, 1H), 6.16 (d, J=16.6 Hz, 1H), 5.68 (d, J=10.4 Hz, 1H), 5.29-5.25 (m, 1H), 4.61-4.21 (m, 3H), 3.98-3.94 (m, 4H), 3.29 (d, J=3.8 Hz, 3H), 2.95 (d, J=3.1 Hz, 3H), 2.63-2.59 (m, 4H), 2.33-2.30 (m, 1H), 1.30 (t, J=7.0 Hz, 3H).

### Example 39: 3-(2-[1-ethyl-2-methylimidazo[4,5-b]pyridin-5-yl]ethynyl)-1-[(3S,5R)-5-(methoxymethyl)-1-(prop-2-enoyl)pyrrolidin-3-yl]-5-(methylamino)pyrazole-4-carboxamide

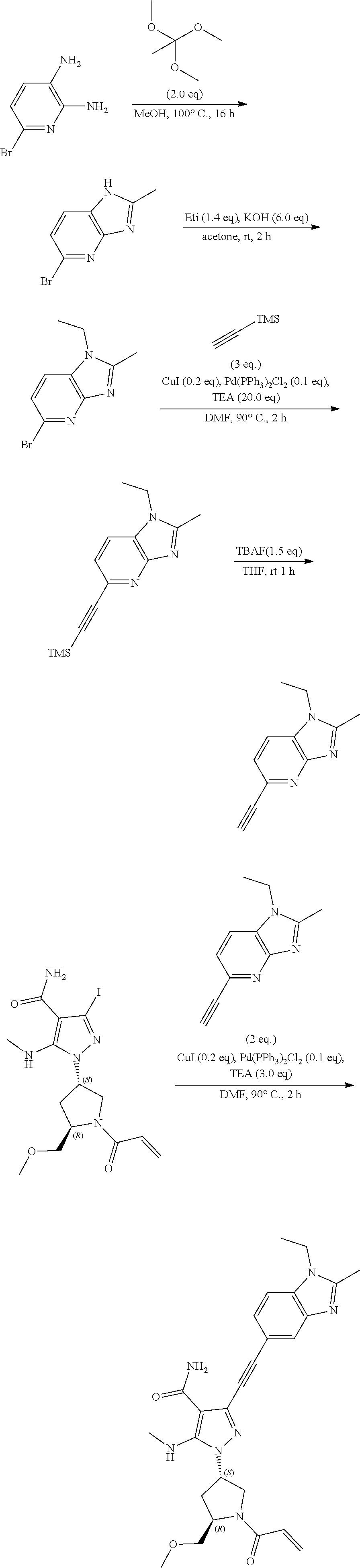

### Step 1: 5-bromo-2-methyl-1H-imidazo[4,5-b]pyridine

To a solution of 6-bromopyridine-2,3-diamine (2.70 g, 14.36 mmol) in AcOH (30.00 mL) was added 1,1,1-trimethoxyethane (3.78 mL, 31.47 mmol). The reaction mixture was stirred for overnight at 100° C. under argon atmosphere. The resulting mixture was concentrated under reduced pressure. The residue was neutralized to pH 7 with saturated NaHCO₃ (aq.). The resulting mixture was extracted with EtOAc (2×200 mL). The combined organic layers were washed with water (2×100 mL), dried over anhydrous Na₂SO₄ and filtered. The filtrate was concentrated under reduced pressure. The residue was purified by silica gel column chromatography, eluted with DCM/MeOH (20/1). The fractions contained desired product were combined and concentrated to afford 5-bromo-2-methyl-1H-imidazo[4,5-b]pyridine (2.4 g, 78%) as an orange solid. MS ESI calculated for C₇H₆BrN₃ [M+H]⁺, 211.97, found 211.90.

### Step 2: 5-bromo-1-ethyl-2-methylimidazo[4,5-b]pyridine

To a stirred mixture of 5-bromo-2-methyl-1H-imidazo[4,5-b]pyridine (2.40 g, 11.32 mmol) and KOH (3.81 g, 67.91 mmol) in acetone (25.00 mL) was added iodoethane (1.27 mL, 8.13 mmol). The reaction mixture was stirred for 2 h at room temperature under argon atmosphere. The resulting mixture was extracted with EtOAc (2×100 mL). The combined organic layers were washed with water (2×100 mL), dried over anhydrous Na₂SO₄ and filtered. The filtrate was concentrated under reduced pressure. The residue was purified by silica gel column chromatography, eluted with CH₂Cl₂/EtOAc (1:2). The fractions contained desired product were combined and concentrated to afford 5-bromo-1-ethyl-2-methylimidazo[4,5-b]pyridine (0.77 g, 28%) as a light yellow solid. MS ESI calculated for C₉H₁₀BrN₃ [M+H]⁺, 240.01, found 240.05.

### Step 3: 1-ethyl-2-methyl-5-[2-(trimethylsilyl)ethynyl]imidazo[4,5-b]pyridine

To a mixture of 5-bromo-1-ethyl-2-methylimidazo[4,5-b]pyridine (0.72 g, 3.00 mmol), trimethylsilylacetylene (0.86 mL, 8.78 mmol), CuI (0.11 g, 0.60 mmol) and Pd(PPh₃)₂Cl₂ (0.21 g, 0.30 mmol) in DMF (10.00 mL) was added TEA (1.30 mL, 12.81 mmol). The reaction mixture was degassed with argon for three times and stirred for 2 h at 90° C. The resulting mixture was concentrated under reduced pressure. The residue was purified by silica gel column chromatography, eluted with PE/EA (1/1). The fractions contained desired product were combined and concentrated to afford 1-ethyl-2-methyl-5-[2-(trimethylsilyl)ethynyl]imidazo[4,5-b]pyridine (0.70 g, 90%) as a brown solid. MS ESI calculated for C₁₄H₁₉N₃Si [M+H]⁺, 258.13, found 258.20.

### Step 4: 1-ethyl-5-ethynyl-2-methylimidazo[4,5-b]pyridine

To a stirred solution of 1-ethyl-2-methyl-5-[2-(trimethylsilyl)ethynyl]imidazo[4,5-b]pyridine (0.70 g, 2.71 mmol) in THF (10.00 mL) were added TBAF (4.06 mL, 4.06 mmol) dropwise at 0° C. under argon atmosphere. The resulting mixture was stirred for 1 h at room temperature. The resulting mixture was concentrated under reduced pressure. The residue was purified by silica gel column chromatography, eluted with CH₂Cl₂/MeOH (20:1). The fractions contained desired product were combined and concentrated to afford 1-ethyl-5-ethynyl-2-methylimidazo[4,5-b]pyridine (0.34 g, 67%) as a light yellow solid. MS ESI calculated for C₁₁H₁₁N₃[M+H]⁺, 186.10, found 186.25.

### Step 5: 3-(2-[1-ethyl-2-methylimidazo[4,5-b]pyridin-5-yl]ethynyl)-1-[(3S,5R)-5-(methoxymethyl)-1-(prop-2-enoyl)pyrrolidin-3-yl]-5-(methylamino)pyrazole-4-carboxamide

To a mixture of 3-iodo-1-[(3S,5R)-5-(methoxymethyl)-1-(prop-2-enoyl)pyrrolidin-3-yl]-5-(methylamino)pyrazole-4-carboxamide (0.15 g, 0.35 mmol), 1-ethyl-5-ethynyl-2-methylimidazo[4,5-b]pyridine (0.13 g, 0.69 mmol), CuI (13.19 mg, 0.07 mmol) and Pd(PPh₃)₂Cl₂ (24.30 mg, 0.04 mmol) in DMF (1.50 mL) was added TEA (0.14 mL, 1.01 mmol). The reaction mixture was degassed with argon for three times and stirred for 2 h at 90° C. The resulting mixture was concentrated under reduced pressure. The residue was purified by Prep-HPLC with the following conditions Column: XBridge Prep OBD C18 Column, 19*250 mm, 5 um; Mobile Phase A: Water (10 mmol/L NH₄HCO₃), Mobile Phase B: ACN; Flow rate: 20 mL/min; Gradient: 35 B to 60 B in 5.8 min; 210/254 nm; RT1: 5.56. The fractions contained desired product were combined and concentrated to afford 3-(2-[1-ethyl-2-methylimidazo[4,5-b]pyridin-5-yl]ethynyl)-1-[(3S,5R)-5-(methoxymethyl)-1-(prop-2-enoyl)pyrrolidin-3-yl]-5-(methylamino)pyrazole-4-carboxamide (43.7 mg, 25%) as an off-white solid. MS ESI calculated for C₂₅H₃₀N₈O₃ [M+H]⁺, 491.24, found 491.25. H-NMR (400 MHz, DMSO-4) S 8.04 (d, J=8.0 Hz, 1H), 7.54-7.48 (m, 2H), 6.90-6.52 (m, 3H), 6.17 (d, J=16.4 Hz, 1H), 5.69 (d, J=10.4 Hz, 1H), 5.29-5.25 (m, 1H), 4.60-4.32 (m, 1H), 4.33-4.24 (m, 2H), 4.09-3.70 (m, 2H), 3.66-3.40 (m, 2H), 3.32-3.27 (m, 1H), 2.99-2.94 (m, 3H), 2.63 (s, 3H), 2.55-2.52 (m, 3H), 2.34-2.30 (m, 1H), 1.42-1.18 (m, 3H).

### Example 40: 3-[2-(6-fluoro-1-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

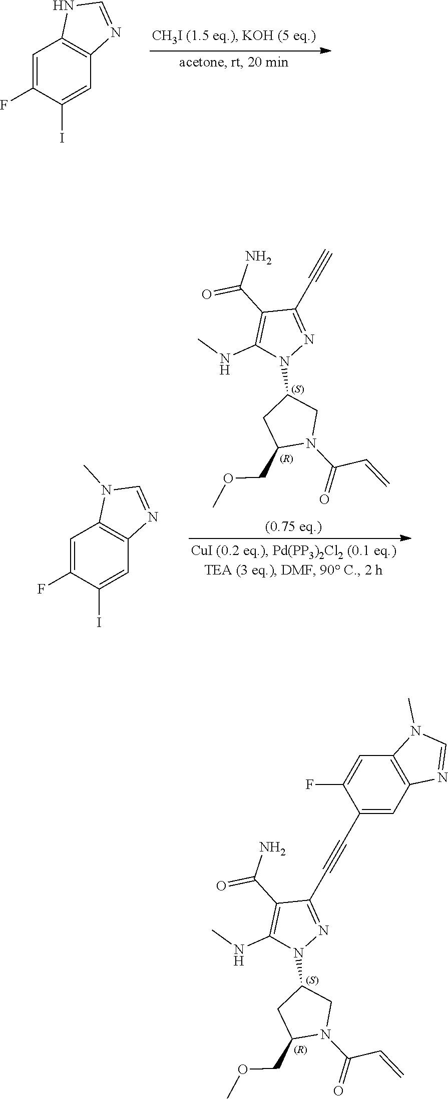

### Step 1: 6-fluoro-5-iodo-1-methyl-1,3-benzodiazole

To a stirred mixture of 5-fluoro-6-iodo-3H-1,3-benzodiazole (0.1 g, 0.38 mmol) and KOH (0.11 g, 1.91 mmol) in acetone was added methyl iodide (0.04 mL) dropwise at 0° C. under nitrogen atmosphere. The reaction mixture was stirred for 20 min at room temperature under nitrogen atmosphere. The resulting mixture was filtered, the filter cake was washed with ethyl acetate (3×10 mL). The filtrate was concentrated under reduced pressure. The residue was purified by silica gel column chromatography, eluted with CH₂Cl₂/MeOH (10:1) to afford crude product. The crude product was purified by Prep-SFC with the following conditions Column: CHIRALPAK IF, 30*250 mm, 5 um; Mobile Phase A: CO₂, Mobile Phase B: MeOH (0.5% 2 M NH₃-MeOH)-HPLC; Flow rate: 80 mL/min; Gradient: 40% B; 220 nm; RT1: 3.8; RT2: 4.59; Injection Volume: 1 ml; Number Of Runs: 40. The fractions contained desired product were combined and concentrated to afford 6-fluoro-5-iodo-1-methyl-1,3-benzodiazole (0.44 g, 41%) as a white solid. ESI calculated for C₈H₆FIN₂ [M+H]⁺, 277; found 277.

### Step 2: 3-[2-(6-fluoro-1-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a stirred mixture of 3-ethynyl-1-[(3S,5R)-5-(methoxymethyl)-1-(prop-2-enoyl)pyrrolidin-3-yl]-5-(methylamino)pyrazole-4-carboxamide (0.15 g, 0.45 mmol), 6-fluoro-5-iodo-1-methyl-1,3-benzodiazole (0.15 g, 0.54 mmol), Pd(PPh₃)₂Cl₂ (31.77 mg, 0.04 mmol) and CuI (17.24 mg, 0.09 mmolv) in DMF (2.00 mL) was added TEA (0.14 g, 1.36 mmol) at room temperature. The reaction mixture was degassed with argon for three times and stirred for 2 h at 90° C. The resulting mixture was concentrated under reduced pressure. The residue was purified by silica gel column chromatography, eluted with CH₂Cl₂/MeOH (10:1). The fractions contained desired product were combined and concentrated. The crude product was purified by Prep-HPLC with the following conditions Column: XBridge Prep C18 OBD Column, 19×150 mm 5 um; Mobile Phase A: Water (10 mmol/L NH₄HCO₃), Mobile Phase B: ACN; Flow rate: 20 mL/min; Gradient: 20 B to 50 B in 4.3 min; 210/254 nm; RT1: 4.23. The fractions contained desired product were combined and concentrated to afford 3-[2-(6-fluoro-1-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (43.6 mg, 20%) as a white solid. ESI calculated for C₂₄H₂₆FN₇O₃[M+H]⁺, 480.21; found 480.15. ¹H NMR (300 MHz, DMSO-d₆) δ 8.30 (s, 1H), 7.93 (d, J=6.2 Hz, 1H), 7.68 (d, J=9.8 Hz, 1H), 7.45 (s, 1H), 6.84-6.50 (m, 3H), 6.16 (d, J=16.9 Hz, 1H), 5.73-5.63 (m, 1H), 5.27-5.23 (m, 1H), 4.49-4.42 (m, 1H), 4.16-3.92 (m, 1H), 3.86-3.80 (m, 6H), 3.65-3.36 (m, 3H), 2.95-2.73 (m, 4H), 2.41-2.15 (m, 1H).

### Example 41: 3-[2-(4-fluoro-1-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

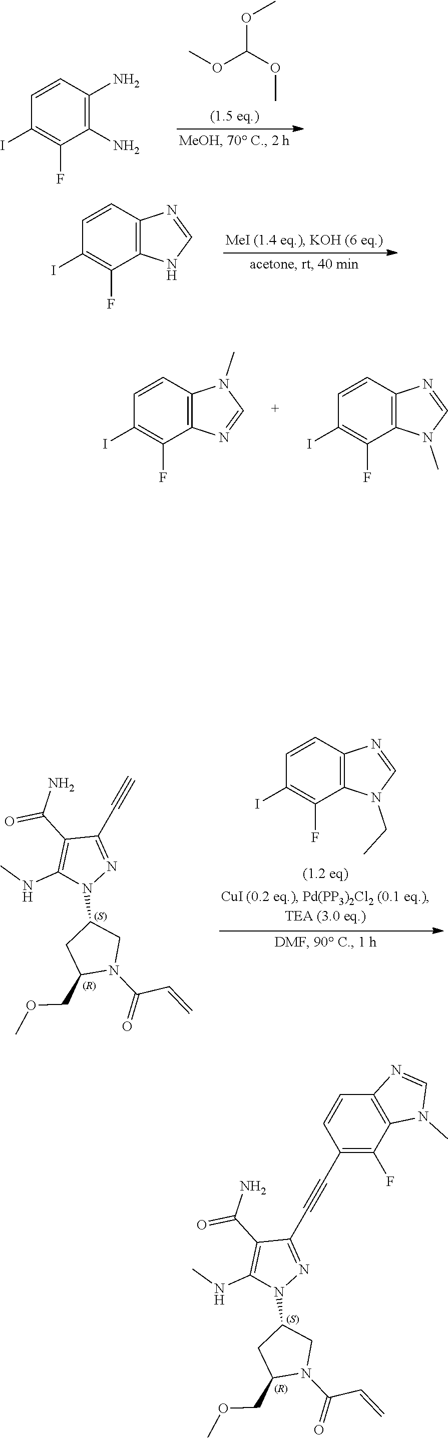

### Step 1: 4-fluoro-5-iodo-1H-1,3-benzodiazole

To a stirred solution of 3-fluoro-4-iodobenzene-1,2-diamine (5.00 g, 19.84 mmol) in MeOH (50.00 mL) was added trimethyl orthoformate (3.16 g, 29.78 mmol) dropwise at room temperature under nitrogen atmosphere. The reaction mixture was stirred for 2 h at 70° C. under nitrogen atmosphere. The resulting mixture was concentrated under reduced pressure. The residue was purified by silica gel column chromatography, eluted with DCM/MeOH (10:1). The fractions contained desired product were combined and concentrated to afford 4-fluoro-5-iodo-1H-1,3-benzodiazole (4.6 g, 88%) as a light yellow solid. MS ESI calculated for C₇H₄FIN₂ [M+H]⁺, 262.94, found 263.00.

### Step 2: 4-fluoro-5-iodo-1-methyl-1,3-benzodiazole and 7-fluoro-6-iodo-1-methyl-1,3-benzodiazole

To a stirred mixture of 4-fluoro-5-iodo-1H-1,3-benzodiazole (3.50 g, 13.36 mmol) and KOH (4.50 g, 80.21 mmol) in acetone (35.00 mL) was added CH₃I (1.16 mL, 8.20 mmol) dropwise at room temperature. The reaction mixture was stirred for 40 min at room temperature. The resulting mixture was diluted with water (40 mL) and extracted with EA (3×70 mL). The combined organic layers were washed with brine (50 mL), dried over anhydrous Na₂SO₄, filtered and concentrated under reduced pressure. The residue was purified by reverse flash chromatography with the following conditions: column, C18 silica gel; mobile phase, ACN in water (NH₄HCO₃ 1 g/L), 20% to 50% gradient in 30 min; detector, UV 254 nm. The fractions contained desired product were combined and concentrated to afford 4-fluoro-5-iodo-1-methyl-1,3-benzodiazole (1.6 g, 43%) as an off-white solid and 7-fluoro-6-iodo-1-methyl-1,3-benzodiazole (1.57 g, 42%) as an off-white solid. MS ESI calculated for C₈H₆FIN₂ [M+H]⁺, 276.96, found 277.00.

### Step 3: 3-[2-(4-fluoro-1-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a stirred mixture of 3-ethynyl-1-[(3S,5R)-5-(methoxymethyl)-1-(prop-2-enoyl)pyrrolidin-3-yl]-5-(methylamino)pyrazole-4-carboxamide (0.15 g, 0.45 mmol) and 4-fluoro-5-iodo-1-methyl-1,3-benzodiazole (0.16 g, 0.54 mmol), CuI (17.24 mg, 0.09 mmol), Pd(PPh₃)₂Cl₂ (31.77 mg, 0.045 mmol) in DMF (2.00 mL) was added TEA (137.41 mg, 1.36 mmol). The reaction mixture was degassed with argon for three times and stirred for 1 h at 90° C. The mixture was allowed to cool down to room temperature and concentrated under reduced pressure. The residue was purified by silica gel column chromatography, eluted with DCM/MeOH (20:1) to afford ˜110 mg product which was further purified was purified by Prep-HPLC with the following conditions: Column: XBridge Prep C18 OBD Column, 19×150 mm 5 um; Mobile Phase A: water (10 mmol/L NH₄HCO₃), Mobile Phase B: ACN; Flow rate: 20 mL/min; Gradient: 30 B to 60 B in 5.8 min; 210/254 nm; RT1:5.56. The fractions contained desired product were concentrated to afford 3-[2-(4-fluoro-1-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (54.7 mg, 25%) as an off-white solid. MS ESI calculated for C₂₄H₂₆FN₇O₃, 479.21, found 480.10. H-NMR (400 MHz, DMSO-d₆) δ 8.32 (s, 1H), 7.53-7.36 (m, 3H), 6.76-6.56 (m, 3H), 6.20-6.14 (m, 1H), 5.71-5.67 (m, 1H), 5.31-5.20 (m, 1H), 4.54-4.30 (m, 1H), 4.04-3.59 (m, 5H), 3.50-3.45 (m, 2H), 3.27 (d, J=2.8 Hz, 3H), 2.96 (t, J=5.2 Hz, 3H), 2.52-2.47 (m, 1H), 2.34-2.36 (m, 1H).

### Example 42: 3-[2-(2-cyclopropyl-1H-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

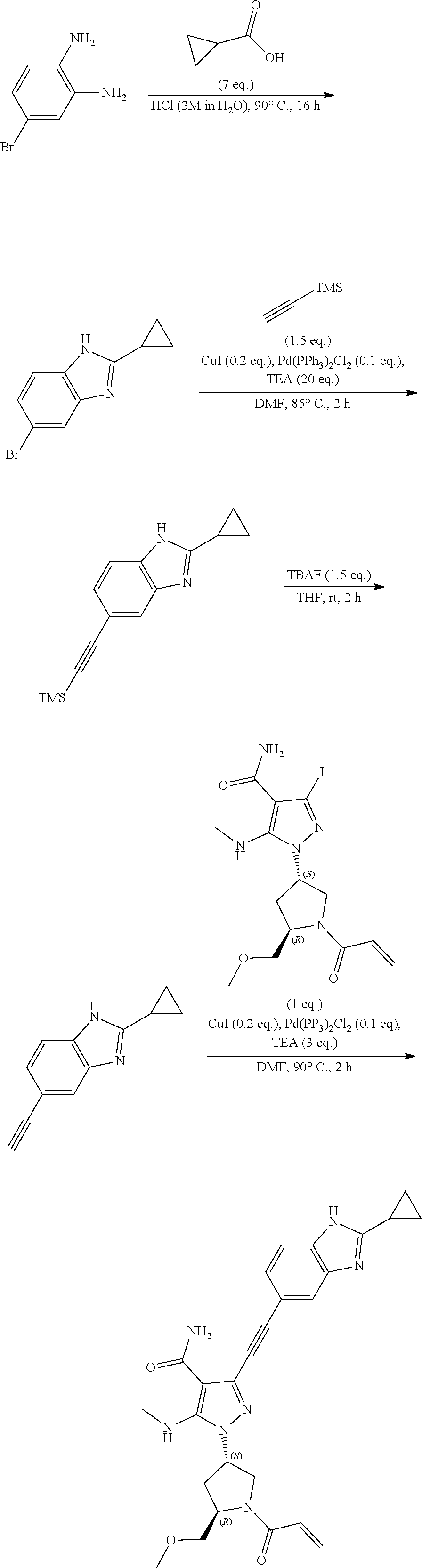

### Step 1: 5-bromo-2-cyclopropyl-1H-benzo[d]imidazole

To a mixture of 4-bromobenzene-1,2-diamine (1.00 g, 5.35 mmol) in HCl (3.00 M in H₂O, 10.00 mL) was added cyclopropanecarboxylic acid (3.22 g, 37.43 mmol). The reaction mixture was stirred for 16 h at 90° C. under nitrogen atmosphere. The resulting mixture was neutralized to pH 7 with NaOH. The mixture was extracted with DCM (3×20 mL). The combined organic layers were washed with water (3×20 mL), dried over anhydrous Na₂SO₄ and filtered. The filtrate was concentrated under reduced pressure. The residue was purified by silica gel column chromatography, eluted with EA in PE (3:2). The fractions contained desired product were combined and concentrated under reduced pressure to afford 5-bromo-2-cyclopropyl-1H-1,3-benzodiazole (1.28 g, 100%) as a yellow solid. MS ESI calculated for C₁₀H₉BrN₂ [M+H]⁺, 236.99, 238.99, found 236.90, 238.90.

### Step 2: 2-cyclopropyl-5-((trimethylsilyl)ethynyl)-1H-benzo[d]imidazole

To a stirred solution of 5-bromo-2-cyclopropyl-1H-1,3-benzodiazole (1.32 g, 5.57 mmol) in DMF (10.00 mL) were added trimethylsilylacetylene (1.64 g, 16.71 mmol), Pd(PPh₃)₂Cl₂ (0.39 g, 0.56 mmol), CuI (0.21 g, 1.11 mmol) and TEA (1.69 g, 16.70 mmol) at room temperature. The reaction mixture was degassed with nitrogen for three times and stirred for 2 h at 85° C. The resulting mixture was diluted with water (100 mL), extracted with EA (3×100 mL). The combined organic layers were washed with brine (3×80 mL), dried over anhydrous Na₂SO₄ and filtered. The filtrate was concentrated under reduced pressure. The residue was purified by silica gel column chromatography, eluted with EA in PE (5:1). The fractions contained desired product were combined and concentrated under reduced pressure to afford 2-cyclopropyl-5-[2-(trimethylsilyl)ethynyl]-1H-1,3-benzodiazole (1.05 g, 74%) as a yellow solid. MS ESI calculated for C₁₅H₁₈N₂Si [M+H]⁺, 255.12; found 255.15.

### Step 3: 2-cyclopropyl-5-ethynyl-1H-benzo[d]imidazole

To a mixture of 2-cyclopropyl-5-[2-(trimethylsilyl)ethynyl]-1H-1,3-benzodiazole (1.05 g, 4.13 mmol) in THF (10.00 mL) was added TBAF (1.62 g, 6.19 mmol). The reaction mixture was stirred for 2 h at room temperature. The resulting mixture was concentrated under reduced pressure. The residue was purified by silica gel column chromatography, eluted with EA in PE (5:1). The fractions contained desired product were combined and concentrated under reduced pressure to afford 2-cyclopropyl-5-ethynyl-1H-1,3-benzodiazole (0.60 g, 79%) as a brown solid. MS ESI calculated for C₁₂H₁₀N₂ [M+H]⁺, 183.08; found 183.00.

### Step 4: 3-[2-(2-cyclopropyl-1H-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a stirred mixture of 3-iodo-1-[(3S,5R)-5-(methoxymethyl)-1-(prop-2-enoyl)pyrrolidin-3-yl]-5-(methylamino)pyrazole-4-carboxamide (0.25 g, 0.58 mmol) and 2-cyclopropyl-5-ethynyl-1H-1,3-benzodiazole (0.11 g, 0.58 mmol) in DMF (2.00 mL) were added Pd(PPh₃)₂Cl₂ (40.50 mg, 0.06 mmol), CuI (21.98 mg, 0.12 mmol) and TEA (0.18 g, 1.73 mmol) at room temperature. The reaction mixture was degassed with argon for three times and stirred for 2 h at 90° C. The resulting mixture was concentrated under reduced pressure. The residue was purified by Prep-HPLC with the following conditions Column: XBridge Prep C18 OBD Column, 19×150 mm 5 um; Mobile Phase A: Water (10 mmol/L NH₄HCO₃), Mobile Phase B: ACN; Flow rate: 20 mL/min; Gradient: 25 B to 65 B in 5.8 min; 210/254 nm; RT1: 5.56 min. The fractions contained desired product were combined and concentrated under reduced pressure to afford 3-[2-(2-cyclopropyl-1H-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (44.20 mg, 15%) as a white solid. MS ESI calculated for C₂₆H₂₉N₇O₃ [M+H]⁺, 488.23, found 488.15. ¹H NMR (400 MHz, DMSO-d₆) δ 12.50 (s, 1H), 7.85-7.14 (m, 4H), 6.94-6.43 (m, 3H), 6.17 (d, J=16.6 Hz, 1H), 5.69 (d, J=10.2 Hz, 1H), 5.27-5.24 (m, 1H), 4.49-4.45 (m, 1H), 4.03-4.00 (m, 1H), 3.89-3.82 (m, 1H), 3.69-5.65 (m, 1H), 3.50-3.46 (m, 1H), 3.32-3.28 (m, 3H), 2.96-2.90 (m, 3H), 2.63-2.57 (m, 1H), 2.31-2.30 (m, 1H), 2.13-2.09 (m, 1H), 1.12-0.90 (m, 4H).

### Example 43: 3-[2-(2-cyclopropyl-1-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

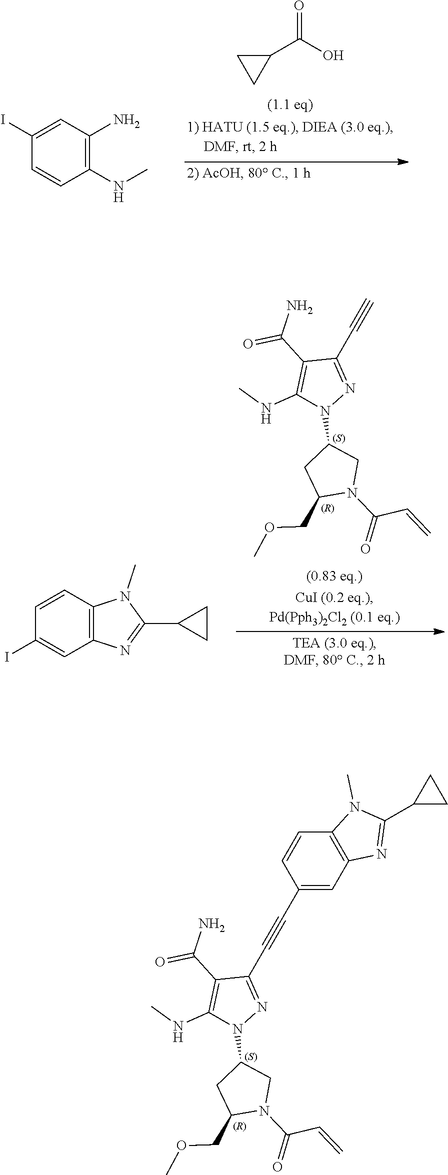

### Step 1: 2-cyclopropyl-5-iodo-1-methyl-1,3-benzodiazole

To a stirred mixture of HATU (4.60 g, 12.09 mmol) and DIEA (3.13 g, 24.18 mmol) in DMF (10.00 mL) was added cyclopropanecarboxylic acid (0.76 g, 8.86 mmol). The reaction mixture was stirred for 30 min at room temperature. To the above mixture was added 4-iodo-N1-methylbenzene-1,2-diamine (2.00 g, 8.06 mmol). The resulting mixture was stirred for 2 h at room temperature. The final reaction mixture was added 50 ml H₂O. The resulting mixture was washed with 5×30 mL of EtOAc. The organic layers was dried over Na₂SO₄ and filtered. The filtrate was concentrated under reduced pressure. To the residue was added AcOH (20.00 mL). The resulting mixture was stirred for 1 h at 80° C. The resulting mixture was concentrated under reduced pressure. The residue was basified to pH 8 with saturated NaHCO₃, extracted with EA (4×150 mL), dried over anhydrous Na₂SO₄ and filtered. The filtrate was concentrated under reduced pressure. The residue was purified by silica gel column chromatography, eluted with EA in PE (0-50%). The fractions contained desired product were combined and concentrated to afford 2-cyclopropyl-5-iodo-1H-1,3-benzodiazole (2.1 g, 87%). MS ESI calculated for C₁₂H₁₂IN [M+H]⁺, 299.00, found 298.90.

### Step 2: 3-[2-(2-cyclopropyl-1-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a stirred mixture 2-cyclopropyl-5-iodo-1-methyl-1,3-benzodiazole (0.11 g, 0.36 mmol) and 3-ethynyl-1-[(3S,5R)-5-(methoxymethyl)-1-(prop-2-enoyl)pyrrolidin-3-yl]-5-(methylamino)pyrazole-4-carboxamide (0.10 g, 0.30 mmol) in DMF (2.00 mL) were added CuI (11.49 mg, 0.06 mmol) and Pd(PPh₃)₂Cl₂ (21.18 mg, 0.03 mmol). The reaction mixture was degassed with argon for three times and stirred for 2 h at 80° C. The resulting mixture was concentrated. The residue was purified by silica gel column chromatography, eluted with DCM/MeOH (0-10%) to afford the crude. The crude was purified by reverse flash chromatography with the following conditions: Column: SunFire Prep C18 OBD Column, 19×150 mm 5 um 10 nm; Mobile Phase A: Water (0.1% FA), Mobile Phase B: ACN; Flow rate: 20 m/min; Gradient: 20 B to 30 B in 6 min; 210/254 nm; RT1: 5.58. The fractions contained desired product were combined and concentrated to afford 3-[2-(2-cyclopropyl-1-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (47.4 mg, 31%). MS ESI calculated for C₂₇H₃₁N₇O₃ [M+H]⁺, 501.25, found 502.15. H NMR (300 MHz, DMSO-d₆) δ 7.74 (d, J=1.4 Hz, 1H), 7.63 (d, J=8.4 Hz, 1H), 7.49-7.26 (m, 2H), 6.77 (s, 1H), 6.71-6.46 (m, 2H), 6.17 (d, J=16.5 Hz, 1H), 5.70 (d, J=10.3 Hz, 1H), 5.39-5.12 (m, 1H), 4.47-4.39 (m, 1H), 4.13-3.95 (m, 1H), 3.93-3.85 (m, 4H), 3.77-3.72 (m, 1H), 3.57-3.52 (m, 2H), 3.33-3.28 (m, 3H), 2.97-2.92 (m, 3H), 2.35-2.32 (m, 2H), 1.21-1.02 (m, 4H).

### Example 44: 1-[(3S,5R)-5-(methoxymethyl)-1-(prop-2-enoyl)pyrrolidin-3-yl]-3-[2-(3-methyl-1,2-benzoxazol-6-yl)ethynyl]-5-(methylamino)pyrazole-4-carboxamide

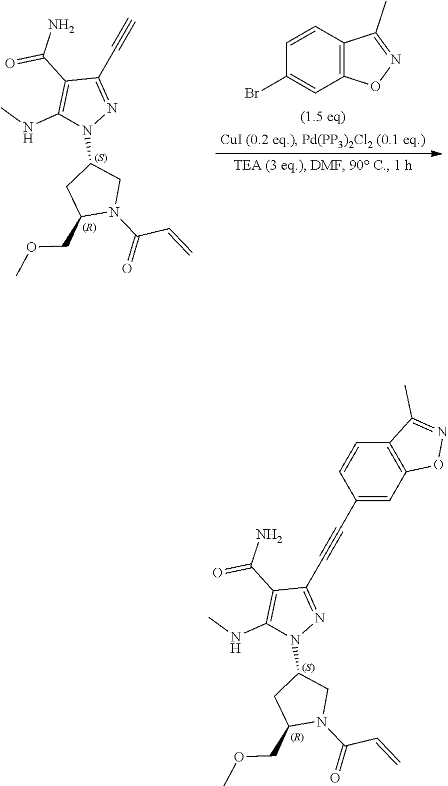

To a stirred mixture of 3-ethynyl-1-[(3S,5R)-5-(methoxymethyl)-1-(prop-2-enoyl)pyrrolidin-3-yl]-5-(methylamino)pyrazole-4-carboxamide (0.23 g, 0.69 mmol), 6-bromo-3-methyl-1,2-benzoxazole (0.22 g, 1.04 mmol), Pd(PPh₃)₂Cl₂ (48.72 mg, 0.07 mmol) and CuI (26.44 mg, 0.14 mmol) in DMF (2.50 mL) was added TEA (0.29 mL, 2.86 mmol). The reaction mixture was degassed with argon for three times and stirred for 1 h at 90° C. The resulting mixture was concentrated under reduced pressure. The residue was purified by silica gel column chromatography, eluted with 6% MeOH in DCM. The fractions contained desired product were combined and concentrated. The crude product (150 mg) was purified by Prep-HPLC with the following conditions Column: XBridge Prep C18 OBD Column, 19×150 mm 5 um; Mobile Phase A: Water (10 mmol/L NH₄HCO₃), Mobile Phase B: ACN; Flow rate: 20 mL/min; Gradient: 30 B to 60 B in 5.8 min; 210/254 nm; RT1: 5.56. The fractions contained desired product were combined and concentrated to afford 1-[(3S,5R)-5-(methoxymethyl)-1-(prop-2-enoyl)pyrrolidin-3-yl]-3-[2-(3-methyl-1,2-benzoxazol-6-yl)ethynyl]-5-(methylamino)pyrazole-4-carboxamide (44.9 mg, 13%) as an off-white solid. MS ESI calculated for C₂₄H₂₆N₆O₄ [M+H]⁺, 463.20, found 463.20. H-NMR (400 MHz, DMSO-d₆): δ 8.04-7.90 (m, 2H), 7.56 (dd, J=8.2, 1.2 Hz, 1H), 7.29-7.23 (m, 1H), 6.94-6.42 (m, 3H), 6.19-6.25 (m, 1H), 5.71-5.67 (m, 1H), 5.26-5.23 (m, 1H), 4.56-4.33 (m, 1H), 4.06-3.67 (m, 2H), 3.64-3.39 (m, 2H), 3.32 (s, 3H), 2.97-2.90 (m, 3H), 2.58 (s, 3H), 2.45-2.41 (m, 1H), 2.35-2.26 (m, 1H).

### Example 45: 3-[2-(2-cyclopropyl-1-ethyl-4,6-difluoro-1,3-benzodiazol-5-yl) ethynyl]-1-[(3S,5R)-5-(methoxymethyl)-1-(prop-2-enoyl) pyrrolidin-3-yl]-5-(methylamino) pyrazole-4-carboxamide

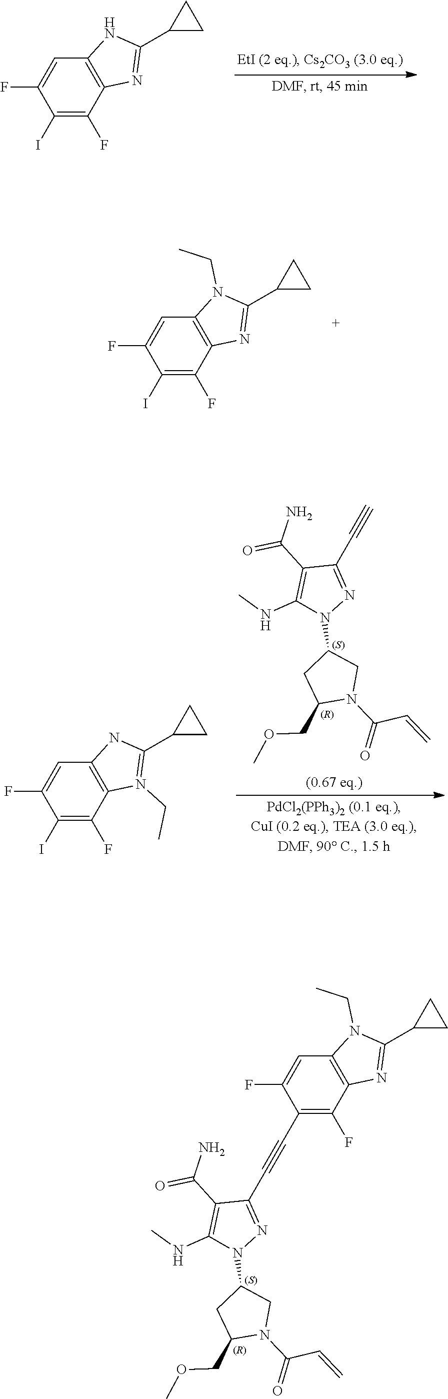

### Step 1: 2-cyclopropyl-1-ethyl-4,6-difluoro-5-iodo-1,3-benzodiazole and 2-cyclopropyl-1-ethyl-5,7-difluoro-6-iodo-1,3-benzodiazole

To a solution of 2-cyclopropyl-4,6-difluoro-5-iodo-1H-1,3-benzodiazole (1.50 g, 4.69 mmol) and iodoethane (1.46 g, 9.37 mmol) in DMF (15.00 mL) was added Cs₂CO₃ (4.58 g, 14.06 mmol) dropwise at room temperature. The reaction mixture was stirred for 45 min at room temperature.

The resulting mixture was filtered, the filtrate was concentrated under reduced pressure. The residue was purified by reverse phase chromatography with the following conditions: Column: Spherical C18, 20-40 um, 300 g; Mobile Phase A: Water (10 mM NH₄HCO₃), Mobile Phase B: ACN; Flow rate: 50 mL/min; Gradient (B %): 5%-45% within 40 min, Detector: UV 254 & 220 nm. The fractions contained desired product were combined and concentrated to afford 2-cyclopropyl-1-ethyl-4,6-difluoro-5-iodo-1,3-benzodiazole (0.50 g, 31%) as a light yellow solid and 2-cyclopropyl-1-ethyl-5,7-difluoro-6-iodo-1,3-benzodiazole (0.37 g, 23%) as a light yellow solid. MS ESI calculated for C₉H₉N₃O₂[M+H]⁺, 348.99, found 348.85.

### Step 2: 3-[2-(2-cyclopropyl-1-ethyl-4,6-difluoro-1,3-benzodiazol-5-yl) ethynyl]-1-[(3S,5R)-5-(methoxymethyl)-1-(prop-2-enoyl) pyrrolidin-3-yl]-5-(methylamino) pyrazole-4-carboxamide

To a stirred mixture of 2-cyclopropyl-1-ethyl-4,6-difluoro-5-iodo-1,3-benzodiazole (0.21 g, 0.60 mmol), 3-ethynyl-1-[(3S,5R)-5-(methoxymethyl)-1-(prop-2-enoyl)pyrrolidin-3-yl]-5-(methylamino)pyrazole-4-carboxamide (0.14 g, 0.45 mmol), CuI (22.98 mg, 0.12 mmol) and Pd(PPh₃)₂Cl₂ (42.34 mg, 0.06 mmol) in DMF (2.00 mL) was added TEA (0.18 g, 1.81 mmol). The reaction mixture was degassed with argon for three times and stirred for 1.5 h at 90° C. The resulting mixture was concentrated under reduced pressure. The residue was purified by reverse phase chromatography with the following conditions: Column: Spherical C18, 20-40 um, 40 g; Mobile Phase A: Water (10 mM NH₄HCO₃), Mobile Phase B: ACN; Flow rate: 40 mL/min; Gradient (B %): 5%-45% within 45 min, Detector: UV 254 & 220 nm. The fractions contained desired product were combined and concentrated to afford 3-[2-(2-cyclopropyl-1-ethyl-4,6-difluoro-1,3-benzodiazol-5-yl)ethynyl]-1-[(3S,5R)-5-(methoxymethyl)-1-(prop-2-enoyl) pyrrolidin-3-yl]-5-(methylamino) pyrazole-4-carboxamide (84.1 mg, 25%) as an off-white solid. MS ESI calculated for C₂₈H₃₁F₂N₇O₃[M+H]⁺, 552.25; found 552.25. ¹H NMR (300 MHz, d₆-DMSO) δ 7.66-7.52 (m, 2H), 6.83-6.52 (m, 3H), 6.19-6.15 (m, 1H), 5.71-5.68 (m, 1H), 5.30-5.27 (m, 1H), 4.58-4.32 (m, 3H), 3.90 (d, J=7.5 Hz, 2H), 3.66-3.41 (m, 2H), 3.34-3.26 (m, 4H), 2.99-2.95 (m, 3H), 2.30-2.25 (m, 2H), 1.35 (t, J=7.1 Hz, 3H), 1.16-1.03 (m, 4H).

### Example 46: 3-[2-[4,6-difluoro-2-(trifluoromethyl)-1H-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide

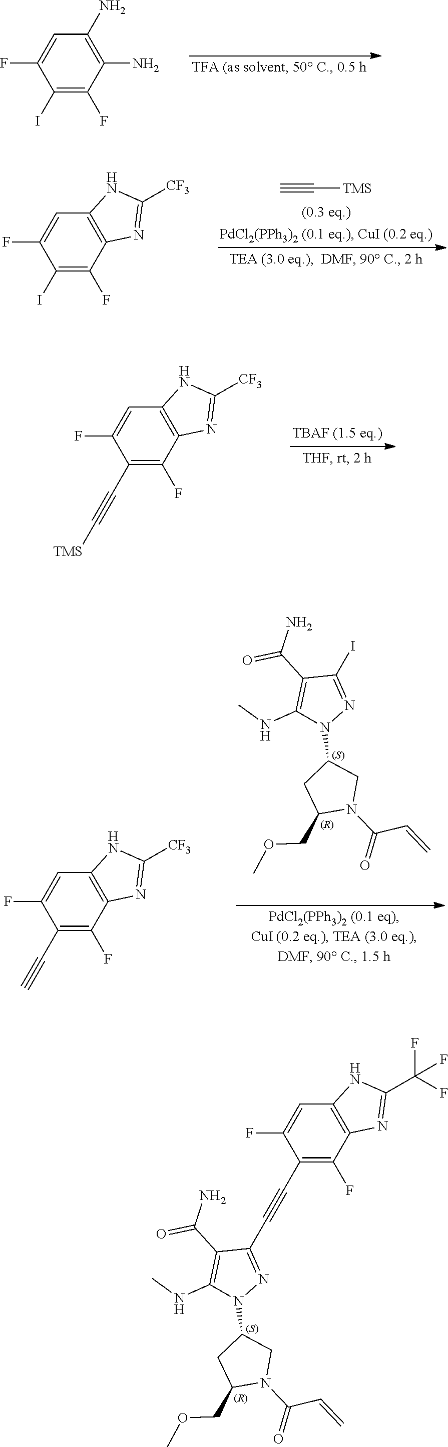

### Step 1: 4,6-difluoro-5-iodo-2-(trifluoromethyl)-1H-1,3-benzodiazole

A solution of 3,5-difluoro-4-iodobenzene-1,2-diamine (5.50 g, 20.37 mmol) in trifluoroacetic acid (55 mL) was stirred at 50° C. for 30 min. The reaction liquid is mixed with saturated NaHCO₃ to basic solution at 0° C. The resulting mixture was poured into Sodium thiosulfate (550.0 mL) and extracted with EA (3×500.0 mL). The combined organic layers were washed with brine (2×300 mL), dried over anhydrous sodium sulfate and filtered. The filtrate was concentrated under reduced pressure. The residue was purified by reverse flash chromatography with the following conditions: Column: Spherical C18, 20-40 um, 40 g; Mobile Phase A: Water (10 mM NH₄HCO₃), Mobile Phase B: ACN; Flow rate: 40 mL/min; Gradient (B %): 0% hold 5 min, 5%-60% within 40 min; 60% hold 5 min, 60%˜95% within 20 min, 95% hold 5 min; Detector: UV 254 & 210 nm; RT: 45 min). The fractions contained desired product were combined and concentrated to afford 4,6-difluoro-5-iodo-2-(trifluoromethyl)-1H-1,3-benzodiazole (1.3 g, 18%) as a light yellow solid. ESI calculated for C₈H₂F₅IN₂ [M+H]⁺, 348.92; found 348.95.

### Step 2: 4,6-Difluoro-2-(trifluoromethyl)-5-[2-(trimethylsilyl)ethynyl]-1H-1,3-benzodiazole

To a stirred solution of 4,6-difluoro-5-iodo-2-(trifluoromethyl)-1H-1,3-benzodiazole (1.00 g, 2.87 mmol) in dimethylformamide (10 mL) were added trimethylsilylacetylene (0.85 g, 8.62 mmol), palladium chloride; bis(triphenylphosphine) (0.20 g, 0.28 mmol), copper(I) iodide (0.11 g, 0.57 mmol) and TEA (0.87 g, 8.62 mmol). The reaction mixture was degassed with argon for three times and stirred for 2 h at 90° C. After cooling down to ambient temperature, the resulting mixture was poured into water (100 mL) and extracted with EA (3×100 mL). The combined organic layers were washed with brine (2×100 mL), dried over anhydrous sodium sulfate and filtered. The filtrate was concentrated under reduced pressure. The residue was purified by silica gel column chromatography, eluted with 18% EA in PE. The fractions contained desired product were combined and concentrated to afford 4,6-difluoro-2-(trifluoromethyl)-5-[2-(trimethylsilyl)ethynyl]-1H-1,3-benzodiazole (0.40 g, 44%) as a light yellow solid. MS ESI calculated for C₁₃H₁₁F₅N₂Si [M+H]⁺, 319.06; found 318.95.

### Step 3: 5-Ethynyl-4,6-difluoro-2-(trifluoromethyl)-1H-1,3-benzodiazole

To a stirred solution of 4,6-difluoro-2-(trifluoromethyl)-5-[2-(trimethylsilyl)ethynyl]-1H-1,3-benzodiazole (0.38 mg, 1.19 mmol) in tetrahydrofuran (5 mL) was added TBAF (1.0 M in THF, 1.80 mL, 1.80 mmol) dropwise at 0° C. The reaction mixture was stirred at room temperature for 2 h. The resulting mixture was poured into sodium thiosulfate (10 mL) and extracted with EA (3×20 mL). The combined organic layers were washed with brine (2×10 mL), dried over anhydrous sodium sulfate and filtered. The filtrate was concentrated under reduced pressure. The residue was purified by Carbon eighteen gel column chromatography, eluted with ACN in H₂O (0˜34%). The fractions contained desired product were combined and concentrated to afford 5-ethynyl-4,6-difluoro-2-(trifluoromethyl)-1H-1,3-benzodiazole (0.21 g, 71%) as a brown solid. MS ESI calculated for C₁₀H₃F₅N₂ [M+H]⁺, 247.02; found 247.14.

### Step 4: 3-[2-[4,6-difluoro-2-(trifluoromethyl)-1H-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide

To a stirred mixture of 5-ethynyl-4,6-difluoro-2-(trifluoromethyl)-1H-1,3-benzodiazole (88.66 mg) and 3-iodo-1-[(3S,5R)-5-(methoxymethyl)-1-(prop-2-enoyl)pyrrolidin-3-yl]-5-(methylamino)pyrazole-4-carboxamide (0.13 g) in DMF (1.3 mL) were added Pd(PPh₃)₂Cl₂ (21.06 mg), CuI (11.4 mg) and TEA (91 mg). The reaction mixture was degassed with argon for three times and stirred for 1.5 h at 90° C. The resulting mixture was purified by reverse flash chromatography with the following conditions: column, C18 silica gel; mobile phase, ACN in water, 5% to 30% gradient in 30 min; detector, UV 210 nm. The fractions contained desired product were combined and concentrated. The crude product was purified by Prep-HPLC with the following conditions Column: XBridge Prep C18 OBD Column, 19×150 mm 5 um; Mobile Phase A: Water (10 mmoL/L NH₄HCO₃), Mobile Phase B: ACN; Flow rate: 20 mL/min; Gradient: 30 B to 60 B in 4.3 min; 210/254 nm; RT1: 4.23. The fractions contained desired product were combined and concentrated to afford 3-[2-[4,6-difluoro-2-(trifluoromethyl)-1H-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide (72 mg, 43%) as a white solid. MS ESI calculated for C₂₄H₂₂F₅N₇O₃[M+H]⁺, 552.17; found 552.48. ¹H NMR (400 MHz, DMSO-d₆) δ 14.88 (s, 1H), 7.58 (brs, 2H), 6.76-6.59 (m, 3H), 6.19-6.14 (m, 1H), 5.71-5.66 (m, 1H), 5.30-5.25 (m, 1H), 4.48-4.44 (m, 1H), 3.99-3.96 (m, 2H), 3.59-3.44 (m, 2H), 3.31 (s, 3H), 2.96 (s, 3H), 2.67-2.60 (m, 1H), 2.32-2.28 (m, 1H).

### Example 47: 3-[2-[1-(difluoromethyl)-4,6-difluoro-2-methyl-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide

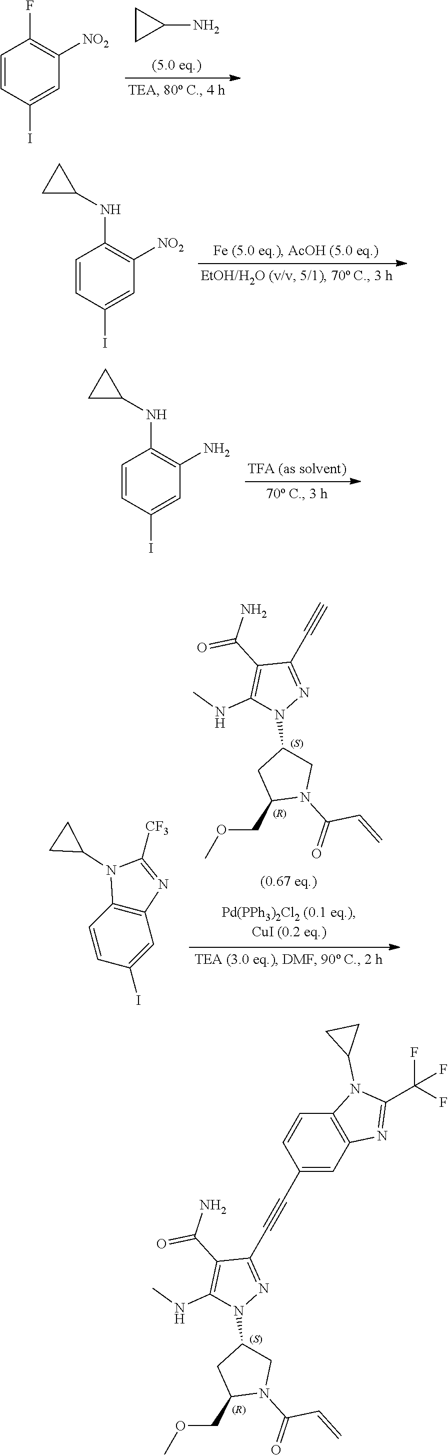

### Step 1: N-cyclopropyl-4-iodo-2-nitroaniline

To a stirred mixture of 1-fluoro-4-iodo-2-nitrobenzene (5.00 g, 18.72 mmol) in Et₃N (14.58 mL, 144.05 mmol) was added aminocyclopropane (5.35 g, 93.63 mmol) at room temperature. The reaction mixture was stirred for 4 h at 80° C. under nitrogen atmosphere. The resulting mixture was cooled. Then the mixture was quenched with sat. NaHCO₃ (30 mL) and extracted with EA (3×50 mL). The combined organic layers were washed with brine (50 mL), dried over anhydrous Na₂SO₄ and filtered. The filtrate was concentrated under reduced pressure. The fractions contained desired product were combined and concentrated under reduced pressure to afford N-cyclopropyl-4-iodo-2-nitroaniline (5.80 g, crude) as a brown solid. MS ESI calculated for C₉H₉IN₂O₂[M+H]⁺, 304.97, found 304.90.

### Step 2: N1-cyclopropyl-4-iodobenzene-1,2-diamine

To a stirred mixture of iron (5.51 g, 98.65 mmol) and AcOH (5.65 mL, 94.14 mmol) in EtOH (50.00 mL) and H₂O (10.00 mL) was added N-cyclopropyl-4-iodo-2-nitroaniline (6.00 g, 19.73 mmol) at 70° C. The resulting reaction was stirred for 3 h at 70° C. The resulting mixture was cooled. Then the mixture was filtered, the filter cake was washed with EA (2×100 mL). The filtrate was concentrated under reduced pressure. The resulting mixture was added water (50 mL) and extracted with EA (3×50 mL). The combined organic layers were washed with brine (2×50 mL), dried over anhydrous Na₂SO₄ and filtered. The filtrate was concentrated under reduced pressure. The residue was purified by silica gel column chromatography, eluted with EA in PE (20-50%). The fractions contained desired product were combined and concentrated under reduced pressure to afford N1-cyclopropyl-4-iodobenzene-1,2-diamine (3.90 g, 72%) as a brown solid. MS ESI calculated for C₉H₁₁IN₂ [M+H]⁺, 275.11, found 275.05.

### Step 3: 1-Cyclopropyl-5-iodo-2-(trifluoromethyl)-1,3-benzodiazole

A solution of N1-cyclopropyl-4-iodobenzene-1,2-diamine (2.00 g, 7.29 mmol) in TFA (15.00 mL) was stirred for 3 h at 70° C. The resulting solution was cooled and concentrated under reduced pressure. The residue was quenched with EA (30 mL) and basified to pH>7 with Sat. NaHCO₃. The mixture was extracted with EA (3×30 mL). The combined organic layers were washed with brine (20 mL), dried over anhydrous Na₂SO₄ and filtered. The filtrate was concentrated under reduced pressure. The residue was purified by silica gel column chromatography, eluted with EA in PE (0-20%). The fractions contained desired product were combined and concentrated to afford 1-cyclopropyl-5-iodo-2-(trifluoromethyl)-1,3-benzodiazole (0.60 g, 23%) as a white solid. MS ESI calculated for C₁₁H₈F₃IN₂ [M+H]⁺, 352.97, found 352.95.

### Step 4: 3-[2-[1-(difluoromethyl)-4,6-difluoro-2-methyl-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide

To a mixture of 3-ethynyl-1-[(3S,5R)-5-(methoxymethyl)-1-(prop-2-enoyl)pyrrolidin-3-yl]-5-(methylamino)pyrazole-4-carboxamide (0.15 g, 0.45 mmol) in DMF (3.00 mL) were added 1-cyclopropyl-5-iodo-2-(trifluoromethyl)-1,3-benzodiazole (0.16 g, 0.45 mmol), Pd(PPh₃)₂Cl₂ (31.77 mg, 0.04 mmol), CuI (17.24 mg, 0.09 mmol) and TEA (137.41 mg, 1.35 mmol). The reaction mixture was degassed with argon for three times and stirred for 2 h at 90° C. The residue was purified by silica gel column chromatography, eluting with MeOH in DCM (0-5%) to afford crude product. Then the crude product was further purified by Prep-HPLC with the following conditions Column: XBridge Prep C18 OBD Column, 19×150 mm 5 um; Mobile Phase A: Water (10 mM NH₄HCO₃), Mobile Phase B: ACN; Flow rate: 20 mL/min; Gradient: 35% B to 70% B in 5.8 min; 210/254 nm. The fractions contained desired product were combined and concentrated to afford 3-[2-[1-cyclopropyl-2-(trifluoromethyl)-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide (51 mg, 22%) as a white solid. MS ESI calculated for C₂₇H₂₈F₃N₇O₃[M+H]⁺, 556.22, found 556.20. ¹H NMR (400 MHz, d₆-DMSO) δ 8.09 (s, 1H), 7.84 (d, J=8.6 Hz, 1H), 7.66-7.64 (m, 1H), 7.38 (brs, 1H), 6.92-6.96 (m, 3H), 6.17-6.14 (m, 1H), 5.69-5.67 (m, 1H), 5.30-5.19 (m, 1H), 4.57-4.52 (m, 1H), 3.91-3.83 (m, 1H), 3.78-3.72 (m, 1H), 3.67-3.58 (m, 2H), 3.53-3.41 (m, 1H), 3.32-3.28 (m, 3H), 2.98-2.92 (m, 3H), 2.49-2.45 (m, 1H), 2.36-2.24 (m, 1H), 1.31-1.12 (m, 4H).

### Example 48: 3-(2-[3-Ethyl-2-methylimidazo[4,5-c]pyridin-6-yl]ethynyl)-1-[(3S,5R)-5-(methoxymethyl)-1-(prop-2-enoyl)pyrrolidin-3-yl]-5-(methylamino)pyrazole-4-carboxamide

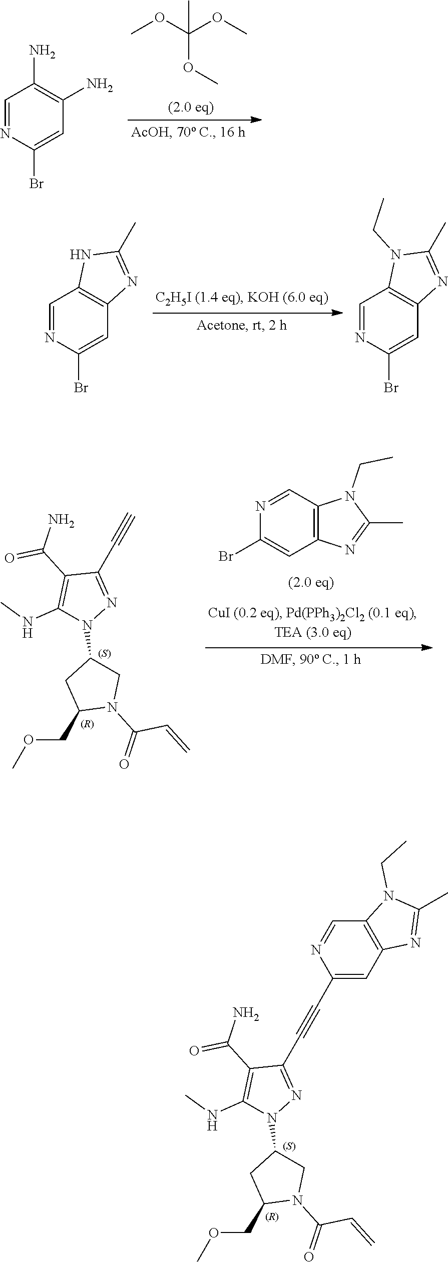

### Step 1: 6-Bromo-2-methyl-3H-imidazo[4,5-c]pyridine

To a stirred solution of 6-bromopyridine-3,4-diamine (1.00 g, 5.32 mmol) in AcOH (10.00 mL) was added 1,1,1-trimethoxyethane (1.36 mL, 10.64 mmol). The reaction mixture was stirred for 16 h at 70° C. under argon atmosphere. The resulting mixture was concentrated under reduced pressure. The residue was neutralized to pH 7 with saturated NaHCO₃ (aq.). The resulting mixture was extracted with EtOAc (2×200 mL). The combined organic layers were washed with water (2×100 mL), dried over anhydrous Na₂SO₄ and filtered. The filtrate was concentrated under reduced pressure. The residue was purified by silica gel Column chromatography, eluted with DCM/MeOH (20/1). The fractions contained desired product were combined and concentrated to afford 6-bromo-2-methyl-3H-imidazo[4,5-c]pyridine (0.86 g, 75%) as a light yellow solid. MS ESI calculated for C₇H₆BrN₃ [M+H]⁺, 211.97, found 212.00.

### Step 2: 6-Bromo-3-ethyl-2-methylimidazo[4,5-c]pyridine

To a stirred mixture of 6-bromo-2-methyl-3H-imidazo[4,5-c]pyridine (0.85 g, 4.01 mmol) and KOH (1.35 g, 24.06 mmol) in acetone (20.00 mL) was added ethyl iodide (0.45 mL, 5.63 mmol) dropwise at 0° C. The resulting mixture was stirred for 2 h at room temperature under argon atmosphere. The resulting mixture was extracted with EtOAc (2×200 mL). The combined organic layers were washed with water (2×100 mL), dried over anhydrous Na₂SO₄ and filtered. The filtrate was concentrated under reduced pressure. The residue was purified by silica gel Column chromatography, eluted with CH₂Cl₂/MeOH (20/1). The fractions contained desired product were combined and concentrated to afford 6-bromo-3-ethyl-2-methylimidazo[4,5-c]pyridine (0.32 g, 33%) as a light yellow solid. ¹H NMR (400 MHz, CDCl₃) δ 8.50 (d, J=0.8 Hz, 1H), 7.78 (d, J=0.8 Hz, 1H), 4.25 (q, J=7.2 Hz, 2H), 2.68 (s, 3H), 1.49 (t, J=7.2 Hz, 3H).

### Step 3: 3-(2-[3-Ethyl-2-methylimidazo[4,5-c]pyridin-6-yl]ethynyl)-1-[(3S,5R)-5-(methoxymethyl)-1-(prop-2-enoyl)pyrrolidin-3-yl]-5-(methylamino)pyrazole-4-carboxamide

To a stirred mixture of 3-ethynyl-1-[(3S,5R)-5-(methoxymethyl)-1-(prop-2-enoyl)pyrrolidin-3-yl]-5-(methylamino)pyrazole-4-carboxamide (0.18 g, 0.54 mmol), 6-bromo-3-ethyl-2-methylimidazo[4,5-c]pyridine (0.16 g, 0.65 mmol), CuI (20.69 mg, 0.11 mmol) and Pd(PPh₃)₂Cl₂ (38.13 mg, 0.05 mmol) in DMF (1.00 mL) was added TEA (0.24 mL, 1.73 mmol). The reaction mixture was degassed with argon for three times and stirred for 1 h at 90° C. The resulting mixture was concentrated under reduced pressure. The residue was purified by Prep-HPLC with the following conditions Column: SunFire Prep C18 OBD Column, 19×150 mm 5 μm 10 nm; Mobile Phase A: Water (0.1% FA), Mobile Phase B: MeOH; Flow rate: 20 mL/min; Gradient: 42 B to 56 B in 6 min; 210/254 nm; RT: 5.56 min. The fractions contained desired product were combined and concentrated to afford 3-(2-[3-ethyl-2-methylimidazo[4,5-c]pyridin-6-yl]ethynyl)-1-[(3S,5R)-5-(methoxymethyl)-1-(prop-2-enoyl)pyrrolidin-3-yl]-5-(methylamino)pyrazole-4-carboxamide (55.3 mg, 20%) as an off-white solid. MS ESI calculated for C₂₅H₃₀N₈O₃ [M+H]⁺, 491.24, found 491.15; ¹H NMR (400 MHz, DMSO-4) S 9.00 (s, 1H), 7.93 (s, 1H), 7.47 (s, 1H), 7.01-6.46 (m, 3H), 6.17 (d, J=16.8 Hz, 1H), 5.69 (d, J=10.4 Hz, 1H), 5.34-5.20 (m, 1H), 4.65-4.25 (m, 3H), 4.09-3.47 (m, 5H), 3.48-3.42 (m, 3H), 2.96 (d, J=4.8 Hz, 3H), 2.64 (s, 3H), 2.37-2.25 (m, 1H), 1.36 (t, J=7.2 Hz, 3H).

### Example 49: 3-[2-(2-Amino-1-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

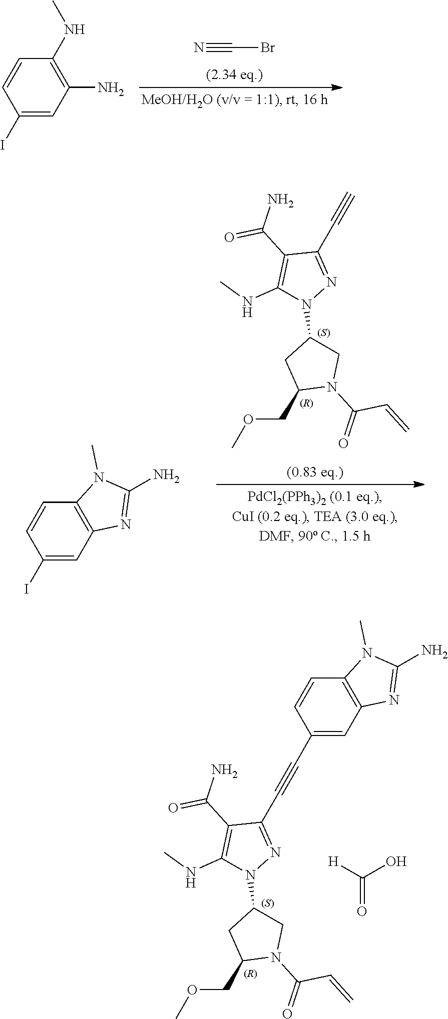

### Step 1: 5-Iodo-1-methyl-1,3-benzodiazol-2-amine

To a stirred solution of 4-iodo-N1-methylbenzene-1,2-diamine (0.60 g, 2.42 mmol) in MeOH (6.00 mL) and H2O (6.00 mL) was added cyanogen bromide (0.60 g, 5.66 mmol). The reaction mixture was stirred for 16 h at room temperature. The resulting mixture was concentrated under reduced pressure. The residue was diluted with water (10 mL) and basified to pH 8 with NaHCO₃ solid. The aqueous layer was extracted with EA (3×15 mL) and the combined organic layers was dried over anhydrous sodium sulfate and filtered. The filtrate was concentrated under reduced pressure. The residue was purified by silica gel Column chromatography, eluting with MeOH in DCM (0-5%). The fractions contained desired product were combined and concentrated under reduced pressure to afford 5-iodo-1-methyl-1,3-benzodiazol-2-amine (0.25 g, 38%). MS ESI calculated for C8H8IN3 [M+H]+, 273.98, found 274.00.

### Step 2: 3-[2-(2-Amino-1-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide; formic acid

To a stirred mixture of 3-ethynyl-1-[(3S,5R)-5-(methoxymethyl)-1-(prop-2-enoyl)pyrrolidin-3-yl]-5-(methylamino)pyrazole-4-carboxamide (0.13 g, 0.39 mmol), 5-iodo-1-methyl-1,3-benzodiazol-2-amine (0.13 g, 0.47 mmol), PdCl2(PPh3)2 (27.54 mg, 0.04 mmol) and CuI (14.94 mg, 0.08 mmol) in DMF (2.00 mL) was added TEA (0.12 g, 1.18 mmol). The reaction mixture was degassed with argon for three times and stirred for 1.5 h at 90° C. The resulting mixture was concentrated under reduced pressure. The residue was purified by silica gel Column chromatography, eluting with MeOH in DCM (0-5.0%) to afford crude product. Then the crude product was further purified by Prep-HPLC with the following conditions: Column: SunFire Prep C18 OBD Column, 19×150 mm 5 μm 10 nm; Mobile Phase A: Water (0.1% FA), Mobile Phase B: MeOH; Flow rate: 20 mL/min; Gradient: 30% B to 50% B in 6 min; 210/254 nm; RT: 5.56 min. The fractions contained desired product were combined and concentrated to afford 3-[2-(2-amino-1-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide; formic acid (30.7 mg, 15%) as a white solid. MS ESI calculated for C24H28N8O3 [M+H−FA]+, 477.24, found 477.25; 1H NMR (400 MHz, DMSO-d6) δ 8.14 (brs. 1H), 7.41-7.21 (m, 2H), 7.18-7.09 (m, 1H), 6.72 (m, 2H), 6.66-6.56 (m, 2H), 6.19-6.15 (m, 1H), 5.70-5.68 (m, 1H), 5.26-5.20 (m, 1H), 4.56-4.36 (m, 1H), 4.05-3.70 (m, 2H), 3.63-3.42 (m, 5H), 3.31-3.27 (m, 3H), 2.96-2.93 (m, 3H), 2.34-2.28 (m, 1H), 2.50-2.45 (m, 1H).

### Example 50: 3-[2-(2-Cyclopropyl-4,6-difluoro-1-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

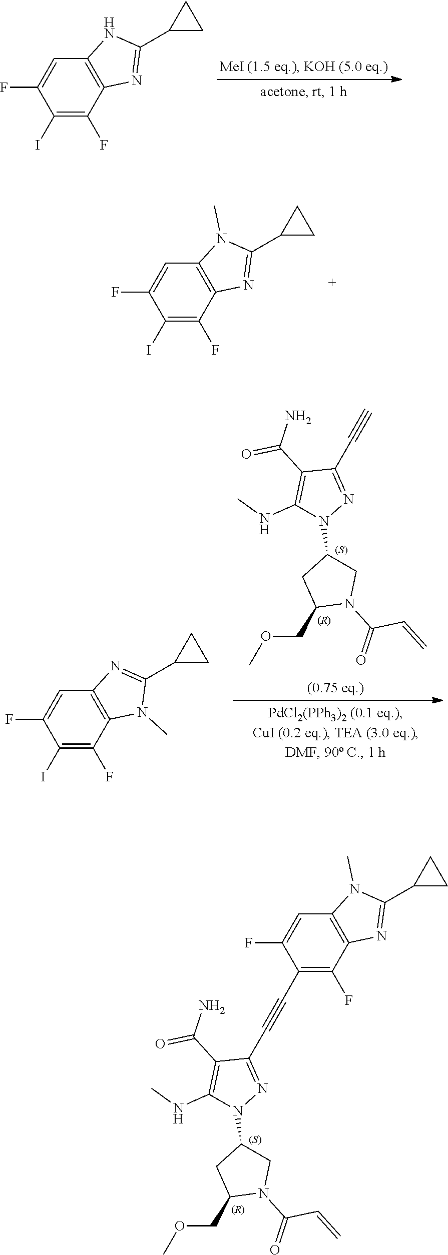

### Step 1: 2-Cyclopropyl-4,6-difluoro-5-iodo-1-methyl-1,3-benzodiazole

To a stirred mixture of 2-cyclopropyl-4,6-difluoro-5-iodo-1H-1,3-benzodiazole (1.20 g, 3.75 mmol) and KOH (1.05 g, 18.75 mmol) in acetone (60.00 mL) was added MeI (1.06 g, 7.50 mmol) dropwise at 0° C. The reaction mixture was stirred for 1 h at room temperature. The resulting mixture was quenched with water (30 mL) at room temperature, extracted with EtOAc (3×60 mL). The combined organic layers were washed with brine (2×30 mL), dried over anhydrous Na2SO4 and filtered. The filtrate was concentrated under reduced pressure. The residue was purified by reverse phase flash with the following conditions Column: Spherical C18, 20-35 μm, 330 g; Mobile Phase A: water (10 mmol/L NH4HCO3), Mobile Phase B: ACN; Flow rate: 85 mL/min; Gradient: 0%-45% within 45 min, Detector: UV 254 & 220 nm; RT: 45 min. The fractions contained desired product were combined and concentrated to afford 2-cyclopropyl-4,6-difluoro-5-iodo-1-methyl-1,3-benzodiazole (0.33 g, 26%) as a light yellow solid. MS ESI calculated for C11H9F2IN2 [M+H]+, 334.98, found 335.00.

### Step 2: 3-[2-(2-Cyclopropyl-4,6-difluoro-1-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a stirred mixture of 2-cyclopropyl-4,6-difluoro-5-iodo-1-methyl-1,3-benzodiazole (0.14 g, 0.40 mmol) in DMF (2.00 mL) were added 3-ethynyl-1-[(3S,5R)-5-(methoxymethyl)-1-(prop-2-enoyl)pyrrolidin-3-yl]-5-(methylamino)pyrazole-4-carboxamide (0.10 g, 0.30 mmol), CuI (15.39 mg, 0.08 mmol), Pd(PPh3)2Cl2 (28.36 mg, 0.04 mmol) and TEA (0.12 g, 1.21 mmol). The reaction mixture was degassed with argon for three times and stirred for 1 h at 90° C. The resulting mixture was concentrated under reduced pressure. The residue was purified by reverse phase chromatography with the following conditions: Column: Spherical C18, 20-40 μm, 40 g; Mobile Phase A: water (10 mmol/L NH4HCO3), Mobile Phase B: ACN; Flow rate: 40 mL/min; Gradient: 20%˜50% within 40 min; Detector: 254/220 nm; RT: 30 min. The fractions contained desired product were combined and concentrated to afford 3-[2-(2-cyclopropyl-4,6-difluoro-1-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (61.3 mg, 28%) as an off-white solid. MS ESI calculated for C27H29F2N703 [M+H]+, 538.23, found 538.30; 1H NMR (300 MHz, DMSO-d6) δ 7.57 (d, J=9.2 Hz, 2H), 6.84-6.54 (m, 3H), 6.18-6.17 (m, 1H), 5.69-5.65 (m, 1H), 5.28-5.26 (m, 1H), 4.58-4.30 (m, 1H), 4.04-4.02 (m, 1H), 3.91-3.57 (m, 5H), 3.49-3.45 (m, 1H), 3.31 (d, J=4.0 Hz, 3H), 2.97-2.94 (m, 3H), 2.68-2.58 (m, 1H), 2.31-2.24 (m, 2H), 1.23-1.02 (m, 4H).

### Example 51: 1-((3S,5R)-1-Acryloyl-5-(methoxymethyl)pyrrolidin-3-yl)-3-((2-cyclopropyl-4-fluoro-1-methyl-1H-benzo[d]imidazol-5-yl)ethynyl)-5-(methylamino)-1H-pyrazole-4-carboxamide

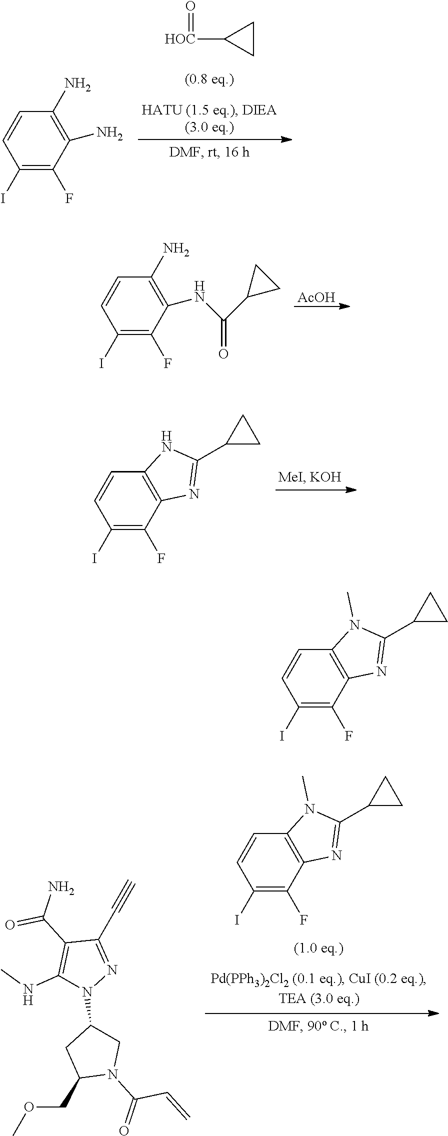

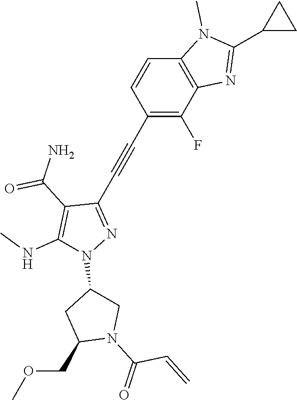 [SMILES: C=CC(=O)N1C[C@@H](n2nc(C#Cc3ccc4c(nc(C5CC5)n4C)c3F)c(C(N)=O)c2NC)C[C@@H]1COC]

### Step 1: N-(6-Amino-2-fluoro-3-iodophenyl)cyclopropanecarboxamide

To a stirred solution of 3-fluoro-4-iodobenzene-1,2-diamine (1.00 g, 3.97 mmol) and HATU (2.26 g, 5.95 mmol), DIEA (2.07 mL, 11.88 mmol) in DMF (7.00 mL) was added cyclopropanecarboxylic acid (0.17 g, 1.98 mmol) in DMF (3.00 mL) dropwise at room temperature under nitrogen atmosphere. The reaction mixture was stirred for 16 h. The resulting mixture was diluted with water (80 mL), extracted with EtOAc (3×80 mL). The combined organic layers were washed with brine (3×60 mL), dried over anhydrous Na2SO4 and filtered. The filtrate was concentrated under reduced pressure. The residue was purified by silica gel Column chromatography, eluted with PE/EtOAc (2/1). The fractions contained desired product were combined and concentrated to afford N-(6-amino-2-fluoro-3-iodophenyl)cyclopropanecarboxamide (0.63 g, 50%) as a brown solid. MS ESI calculated for C10H10FIN2O [M+H]+, 320.98, found 321.05.

### Step 2: 2-Cyclopropyl-4-fluoro-5-iodo-1H-benzo[d]imidazole

A solution of N-(6-amino-2-fluoro-3-iodophenyl)cyclopropanecarboxamide (0.63 g, 1.97 mmol) in AcOH (6.00 mL) was stirred for 5 h at 70° C. under nitrogen atmosphere. The resulting mixture was concentrated under reduced pressure. The residue was neutralized to pH 8 with saturated NaHCO₃ (aq.). The resulting mixture was extracted with EtOAc (3×40 mL). The combined organic layers were washed with brine (3×30 mL), dried over anhydrous Na2SO4 and filtered. The filtrate was concentrated under reduced pressure. The residue was purified by silica gel Column chromatography, eluted with PE/EtOAc (1/1). The fractions contained desired product were combined and concentrated to afford 2-cyclopropyl-4-fluoro-5-iodo-1H-benzo[d]imidazole (0.41 g, 69%) as an off-white solid. MS ESI calculated for C10H8FIN2 [M+H]+, 302.97, found 302.95.

### Step 3: 2-Cyclopropyl-4-fluoro-5-iodo-1-methyl-1H-benzo[d]imidazole

To a stirred mixture of 2-cyclopropyl-4-fluoro-5-iodo-1H-benzo[d]imidazole (0.38 g, 1.26 mmol) and KOH (0.42 g, 7.55 mmol) in acetone (5.00 mL) was added methyl iodide (0.25 g, 1.76 mmol) dropwise at 0° C. under nitrogen atmosphere. The reaction mixture was stirred for 40 min at room temperature. The resulting mixture was concentrated under reduced pressure. The residue was purified by reverse flash chromatography with the following conditions: Column, C18 silica gel; mobile phase A: Water (10 mmol/L NH4HCO3), Mobile Phase B: ACN; Gradient: 30 B to 60 B in 20 min; detector, UV 254 nm. The fractions contained desired product were combined and concentrated to afford 2-cyclopropyl-4-fluoro-5-iodo-1-methyl-1H-benzo[d]imidazole (0.14 g, 35%) as an off-white solid. MS ESI calculated for C11H10FIN2 [M+H]+, 316.99, found 316.95.

### Step 4: 1-((3S,5R)-1-Acryloyl-5-(methoxymethyl)pyrrolidin-3-yl)-3-((2-cyclopropyl-4-fluoro-1-methyl-1H-benzo[d]imidazol-5-yl)ethynyl)-5-(methylamino)-1H-pyrazole-4-carboxamide

To a stirred mixture of 3-ethynyl-1-[(3S,5R)-5-(methoxymethyl)-1-(prop-2-enoyl)pyrrolidin-3-yl]-5-(methylamino)pyrazole-4-carboxamide (0.13 g, 0.39 mmol), 2-cyclopropyl-4-fluoro-5-iodo-1-methyl-1H-benzo[d]imidazole (0.12 g 0.38 mmol), CuI (14.83 mg, 0.08 mmol) and Pd(PPh₃)₂Cl₂ (27.32 mg, 0.04 mmol) in DMF (1.50 mL) was added TEA (0.16 mL, 1.15 mmol). The reaction mixture was degassed with argon for three times and stirred for 1 h at 90° C. The resulting mixture was concentrated under reduced pressure. The residue was purified by Prep-HPLC with the following conditions: Column: XBridge Prep C18 OBD Column, 19×150 mm 5 μm; Mobile Phase A: Water (10 mmol/L NH4HCO3), Mobile Phase B: ACN; Flow rate: 20 mL/min; Gradient: 30 B to 60 B in 5.8 min; 210/254 nm; RT: 5.56 min. The fractions contained desired product were combined and concentrated to afford 1-((3S,5R)-1-acryloyl-5-(methoxymethyl)pyrrolidin-3-yl)-3-((2-cyclopropyl-4-fluoro-1-methyl-1H-benzo[d]imidazol-5-yl)ethynyl)-5-(methylamino)-1H-pyrazole-4-carboxamide (39.4 mg, 20%) as an off-white solid. MS ESI calculated for C27H30FN7O3 [M+H]+, 520.58, found 520.25; 1H NMR (400 MHz, DMSO-d6) δ 7.44-7.38 (m, 3H), 6.84-6.71 (m, 3H), 6.19-6.15 (m, 1H), 5.70-5.68 (m, 1H), 5.29-5.21 (m, 1H), 4.53 (d, J=54.3 Hz, 1H), 4.07-3.98 (m, 1H), 3.91-3.87 (m, 4H), 3.66-3.40 (m, 2H), 3.32-3.30 (m, 3H), 2.97 (t, J=5.2 Hz, 3H), 2.33-2.28 (m, 3H), 1.17-1.11 (m, 4H).

### Example 52: 1-[(3S,5R)-5-(Methoxymethyl)-1-(prop-2-enoyl) pyrrolidin-3-yl]-5-(methylamino)-3-[2-(quinolin-7-yl) ethynyl]pyrazole-4-carboxamide

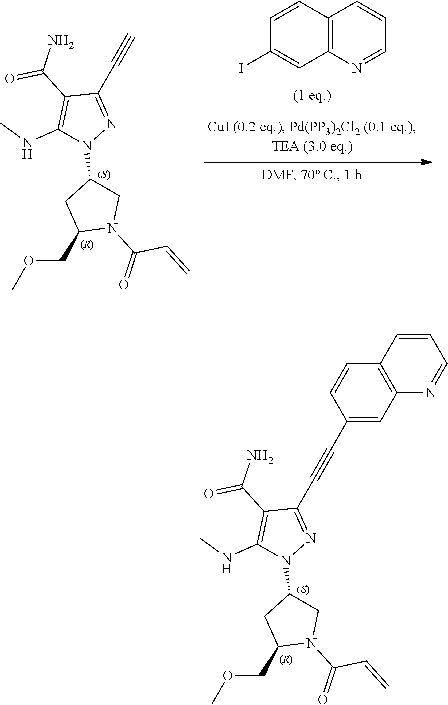

To a mixture of 7-iodoquinoline (0.1 g, 0.39 mmol), 3-ethynyl-1-[(3S,5R)-5-(methoxymethyl)-1-(prop-2-enoyl) pyrrolidin-3-yl]-5-(methylamino) pyrazole-4-carboxamide (0.13 g, 0.39 mmol), CuI (14.93 mg, 0.07 mmol) and Pd(PPh₃)₂Cl₂ (27.52 mg, 0.04 mmol) in DMF (2.00 mL) was added TEA (0.12 g, 1.17 mmol). The reaction mixture was degassed with argon for three times and stirred for 1 h at 70° C. The resulting mixture was concentrated under reduced pressure. The residue was purified by reverse phase chromatography with the following conditions: Column: Spherical C18, 20-40 μm, 40 g; Mobile Phase A: Water (10 mM NH4HCO3), Mobile Phase B: ACN; Flow rate: 40 mL/min; Gradient: 15%-40% within 30 min, Detector: UV 254/220 nm; RT: 30 min. The fractions contained desired product were combined and concentrated to afford 1-[(3S,5R)-5-(methoxymethyl)-1-(prop-2-enoyl) pyrrolidin-3-yl]-5-(methylamino)-3-[2-(quinolin-7-yl) ethynyl]pyrazole-4-carboxamide (0.10 g, 47%) as an off-white solid. MS ESI calculated for C25H26N6O3 [M+H]+, 459.21, found 459.20; 1H NMR (300 MHz, DMSO-d6) δ 8.98 (dd, J=4.2, 1.7 Hz, 1H), 8.46-8.37 (m, 1H), 8.24 (s, 1H), 8.06 (d, J=8.5 Hz, 1H), 7.73 (dd, J=8.5, 1.7 Hz, 1H), 7.60 (dd, J=8.3, 4.2 Hz, 1H), 7.37 (s, 1H), 6.92 (s, 1H), 6.80-6.48 (m, 2H), 6.18-6.15 (m, 1H), 5.73-5.68 (m, 1H), 5.30-5.21 (m, 1H), 4.49-4.45 (m 1H), 4.09-3.70 (m, 2H), 3.66-3.41 (m, 2H), 3.32 (d, J=3.6 Hz, 3H), 2.95-2.93 (m, 3H), 2.67-2.53 (m, 1H), 2.38-2.26 (m, 1H).

### Example 53: 1-[(3S,5R)-5-(Methoxymethyl)-1-(prop-2-enoyl)pyrrolidin-3-yl]-5-(methylamino)-3-(2-[pyrazolo[1,5-a]pyrimidin-5-yl]ethynyl)pyrazole-4-carboxamide

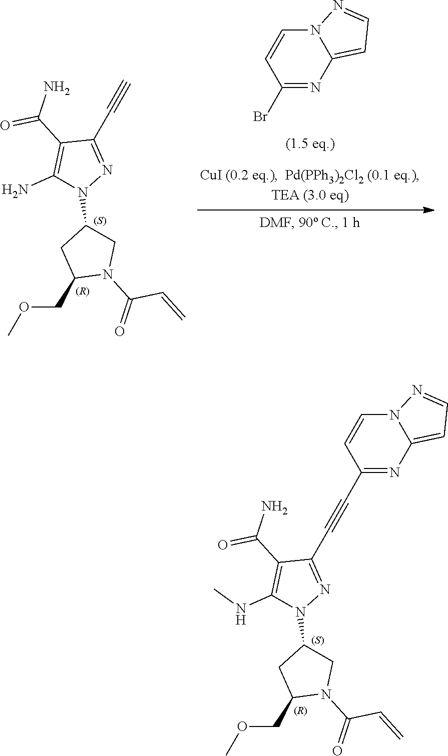

To a stirred mixture of 5-amino-3-ethynyl-1-[(3S,5R)-5-(methoxymethyl)-1-(prop-2-enoyl)pyrrolidin-3-yl]pyrazole-4-carboxamide (0.15 g, 0.47 mmol) and 5-bromopyrazolo[1,5-a]pyrimidine (0.12 g, 0.61 mmol) in DMF (2.00 mL) were added CuI (18.00 mg, 0.10 mmol), Pd(PPh3)2Cl2 (33.18 mg, 0.05 mmol) and TEA (0.14 g, 1.42 mmol) at room temperature. The reaction mixture was degassed with argon for three times and stirred for 1 h at 90° C. The resulting mixture was concentrated under reduced pressure. The residue was purified by silica gel Column chromatography, eluted with CH2Cl2/MeOH (0%-5%). The fractions contained desired product were combined and concentrated. The residue was purified by Prep-HPLC with the following conditions: Column: XBridge Prep C18 OBD Column, 19×150 mm 5 μm; Mobile Phase A: Water (10 mmol/L NH4HCO3), Mobile Phase B: ACN; Flow rate: 20 mL/min; Gradient: 35 B to 55 B in 6 min; 210/254 nm. The fractions contained desired product were combined and concentrated to afford 1-[(3S,5R)-5-(methoxymethyl)-1-(prop-2-enoyl)pyrrolidin-3-yl]-5-(methylamino)-3-(2-[pyrazolo[1,5-a]pyrimidin-5-yl]ethynyl)pyrazole-4-carboxamide (69.6 mg, 32%) as a yellow solid. MS ESI calculated for C22H24N8O3, 448.20, found 448.48; 1H NMR (400 MHz, DMSO-d6) δ 9.19 (dd, J=7.3, 1.0 Hz, 1H), 8.32 (d, J=2.4 Hz, 1H), 7.44 (s, 1H), 7.22 (dd, J=7.3, 3.6 Hz, 1H), 6.91-6.78 (m, 2H), 6.64-6.46 (m, 2H), 6.18-6.16 (m, 1H), 5.71-5.68 (m, 1H), 5.24-5.22 (m, 1H), 4.23-4.21 (m, 1H), 3.92-3.82 (m, 2H), 3.74-3.72 (m, 1H), 3.62-3.59 (m, 1H), 3.53-3.42 (m, 3H), 2.94 (t, J=5.3 Hz, 3H), 2.62-2.59 (m, 1H), 2.46-2.43 (m, 1H).

### Example 54: 1-((3S,5R)-1-Acryloyl-5-(methoxymethyl)pyrrolidin-3-yl)-3-(imidazo[1,2-a]pyridin-7-ylethynyl)-5-(methylamino)-1H-pyrazole-4-carboxamide

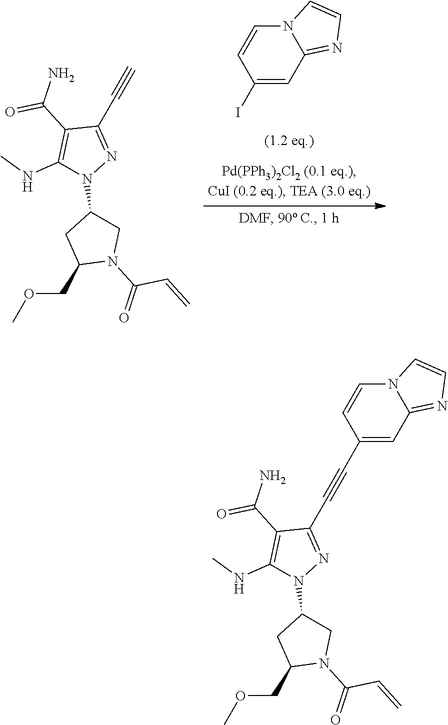 [SMILES: C#Cc1nn([C@H]2C[C@H](COC)N(C(=O)C=C)C2)c(NC)c1C(N)=O ; Ic1ccn2ccnc2c1 ; C=CC(=O)N1C[C@@H](n2nc(C#Cc3ccn4ccnc4c3)c(C(N)=O)c2NC)C[C@@H]1COC]

To a stirred mixture of 3-ethynyl-1-[(3S,5R)-5-(methoxymethyl)-1-(prop-2-enoyl)pyrrolidin-3-yl]-5-(methylamino)pyrazole-4-carboxamide (0.13 g, 0.39 mmol), 7-iodoimidazo[1,2-a]pyridine (0.12 g, 0.47 mmol), CuI (14.94 mg, 0.08 mmol) and Pd(PPh3)2Cl2 (27.54 mg, 0.04 mmol) in DMF (1.50 mL) was added TEA (0.16 mL, 1.15 mmol). The reaction mixture was degassed with argon for three times and stirred for 1 h at 90° C. The resulting mixture was diluted with water (20 mL), extracted with EA (3×20 mL). The combined organic layers were washed with brine (3×20 mL), dried over anhydrous Na2SO4 and filtered. The filtrate was concentrated under reduced pressure. The residue was purified by Prep-HPLC with the following conditions: Column: XBridge Prep C18 OBD Column, 19×150 mm 5 μm; Mobile Phase A: Water (10 mmol/L NH4HCO3), Mobile Phase B: ACN; Flow rate: 20 mL/min; Gradient: 30 B to 60 B in 6 min; 210/254 nm; RT: 5.56 min. The fractions contained desired product were combined and concentrated to afford 1-((3S,5R)-1-acryloyl-5-(methoxymethyl)pyrrolidin-3-yl)-3-(imidazo[1,2-a]pyridin-7-ylethynyl)-5-(methylamino)-1H-pyrazole-4-carboxamide (67.0 mg, 38%) as a light pink solid. MS ESI calculated for C23H25N7O3 [M+H]+, 448.20, found 448.20; 1H NMR (400 MHz, DMSO-d6) δ 8.68 (s, 1H), 8.17-7.88 (m, 3H), 7.32 (s, 1H), 7.09-6.45 (m, 4H), 6.19-6.14 (m, 1H), 5.71-5.67 (m, 1H), 5.27-5.19 (m, 1H), 4.54-4.39 (m, 1H), 4.07-3.68 (m, 2H), 3.64-3.38 (m, 2H), 3.32 (s, 3H), 2.94 (t, J=5.1 Hz, 3H), 2.63-2.56 (m, 1H), 2.34-2.24 (m, 1H).

### Example 55: 1-((3S,5R)-1-Acryloyl-5-(methoxymethyl)pyrrolidin-3-yl)-3-(imidazo[1,2-a]pyridin-6-ylethynyl)-5-(methylamino)-1H-pyrazole-4-carboxamide

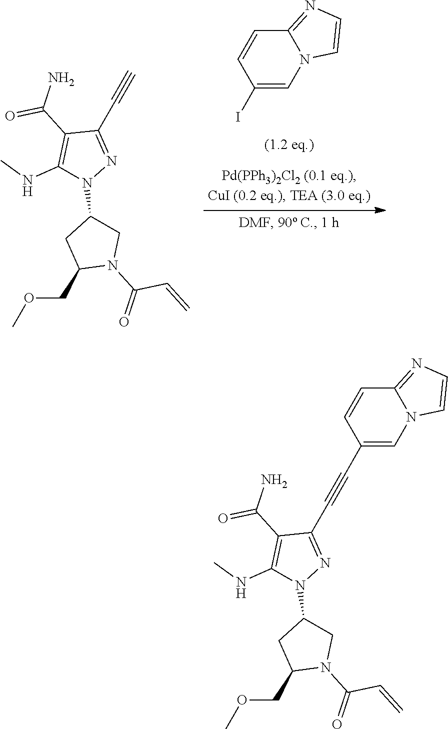 [SMILES: C#Cc1nn([C@H]2C[C@H](COC)N(C(=O)C=C)C2)c(NC)c1C(N)=O ; Ic1ccc2nccn2c1 ; C=CC(=O)N1C[C@@H](n2nc(C#Cc3ccc4nccn4c3)c(C(N)=O)c2NC)C[C@@H]1COC]

To a stirred mixture of 3-ethynyl-1-[(3S,5R)-5-(methoxymethyl)-1-(prop-2-enoyl)pyrrolidin-3-yl]-5-(methylamino)pyrazole-4-carboxamide (0.13 g, 0.39 mmol), 6-iodoimidazo[1,2-a]pyridine (0.11 g, 0.47 mmol), CuI (14.94 mg, 0.08 mmol) and Pd(PPh₃)₂Cl₂ (27.54 mg, 0.04 mmol) in DMF (1.50 mL) was added TEA (0.16 mL, 1.15 mmol). The reaction mixture was degassed with argon for three times and stirred for 1 h at 90° C. The resulting mixture was diluted with water (20 mL), extracted with EA (3×20 mL). The combined organic layers were washed with brine (3×20 mL), dried over anhydrous Na2SO4 and filtered. The filtrate was concentrated under reduced pressure. The residue was purified by Prep-HPLC with the following conditions: Column: XBridge Prep C18 OBD Column, 19×150 mm 5 μm; Mobile Phase A: Water (10 mmol/L NH4HCO3), Mobile Phase B: ACN; Flow rate: 20 mL/min; Gradient: 25 B to 55 B in 6 min; 210/254 nm; RT: 5.52 min. The fractions contained desired product were combined and concentrated to afford 1-((3S,5R)-1-acryloyl-5-(methoxymethyl)pyrrolidin-3-yl)-3-(imidazo[1,2-a]pyridin-6-ylethynyl)-5-(methylamino)-1H-pyrazole-4-carboxamide (82.8 mg, 47%) as an off-white solid. MS ESI calculated for C23H25N7O3 [M+H]+, 448.20, found 448.10; 1H NMR (400 MHz, DMSO-d6) δ 9.16 (s, 1H), 8.41-7.39 (m, 3H), 7.35 (s, 2H), 7.02-6.42 (m, 3H), 6.19-6.13 (m, 1H), 5.71-5.67 (m, 1H), 5.29-5.17 (m, 1H), 4.54-4.39 (m, 1H), 4.13-3.65 (m, 2H), 3.64-3.40 (m, 2H), 3.30 (s, 3H), 2.94 (t, J=5.2 Hz, 3H), 2.69-2.54 (m, 1H), 2.36-2.23 (m, 1H).

### Example 56: 1-[(3S,5R)-5-(Methoxymethyl)-1-(prop-2-enoyl)pyrrolidin-3-yl]-5-(methylamino)-3-(2-[pyrazolo[1,5-a]pyridin-5-yl]ethynyl)pyrazole-4-carboxamide

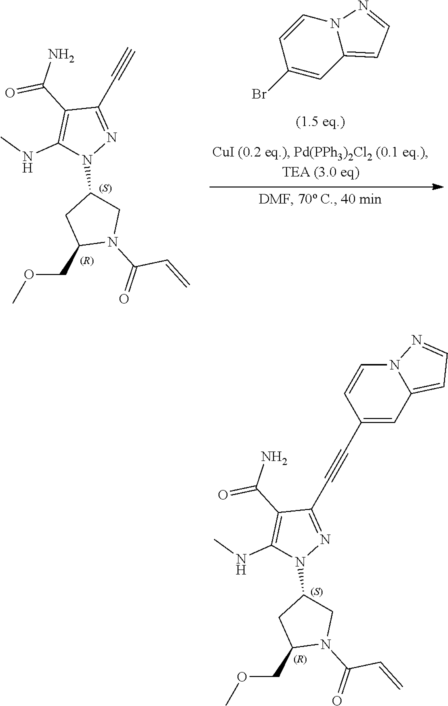

To a stirred mixture of 3-ethynyl-1-[(3S,5R)-5-(methoxymethyl)-1-(prop-2-enoyl)pyrrolidin-3-yl]-5-(methylamino)pyrazole-4-carboxamide (0.18 g, 0.54 mmol), 5-bromopyrazolo[1,5-a]pyridine (0.16 g, 0.82 mmol), Pd(PPh3)2Cl2 (38.13 mg, 0.05 mmol) and CuI (20.69 mg, 0.11 mmol) in DMF (2.00 mL) was added TEA (0.23 mL, 2.24 mmol). The reaction mixture was degassed with argon for three times and stirred for 40 min at 70° C. The resulting mixture was concentrated under reduced pressure. The residue was purified by silica gel Column chromatography, eluted with CH2Cl2/MeOH (10/1). The fractions contained desired product were combined and concentrated. The residue was purified by Prep-HPLC with the following conditions Column: XBridge Prep C18 OBD Column, 19×150 mm 5 μm; Mobile Phase A: Water (10 mmol/L NH4HCO3), Mobile Phase B: ACN; Flow rate: 20 mL/min; Gradient: 25 B to 55 B in 6 min; 210/254 nm; RT: 5.56 min. The fractions contained desired product were combined and concentrated to afford 1-[(3S,5R)-5-(methoxymethyl)-1-(prop-2-enoyl)pyrrolidin-3-yl]-5-(methylamino)-3-(2-[pyrazolo[1,5-a]pyridin-5-yl]ethynyl)pyrazole-4-carboxamide (66.6 mg, 27%) as an off-white solid. MS ESI calculated for C23H25N7O3 [M+H]+, 448.20, found 448.15; 1H NMR (400 MHz, DMSO-d6): δ 8.74 (d, J=7.3 Hz, 1H), 8.13-7.97 (m, 2H), 7.34-7.32 (m, 1H), 6.99-6.45 (m, 5H), 6.17 (d, J=16.5 Hz, 1H), 5.76-5.62 (m, 1H), 5.26-5.23 (m, 1H), 4.49-4.39 (m, 1H), 4.09-3.67 (m, 2H), 3.63-3.38 (m, 2H), 3.31 (d, J=4.1 Hz, 3H), 2.95 (d, J=4.7 Hz, 3H), 2.65-2.61 (m, 1H), 2.39-2.24 (m, 1H).

### Example 57: 3-(2-[Imidazo[1,2-a]pyrimidin-6-yl]ethynyl)-1-[(3S,5R)-5-(methoxymethyl)-1-(prop-2-enoyl)pyrrolidin-3-yl]-5-(methylamino)pyrazole-4-carboxamide

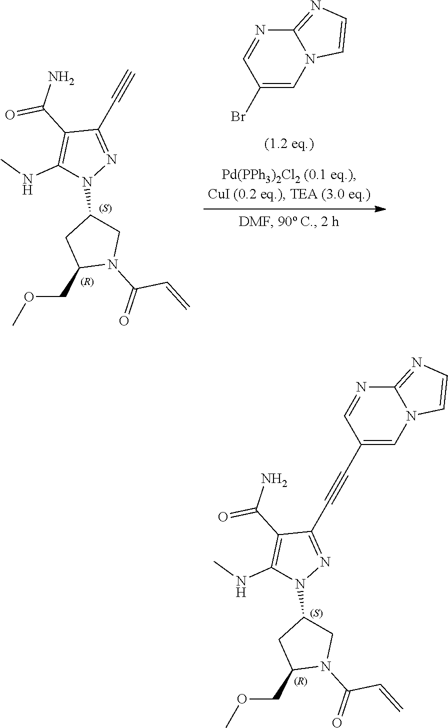

To a stirred mixture of 3-ethynyl-1-[(3S,5R)-5-(methoxymethyl)-1-(prop-2-enoyl)pyrrolidin-3-yl]-5-(methylamino)pyrazole-4-carboxamide (0.13 g, 0.39 mmol), 6-bromoimidazo[1,2-a]pyrimidine (93.22 mg, 0.47 mmol), Pd(PPh₃)₂Cl₂ (27.54 mg, 0.04 mmol) and CuI (14.94 mg, 0.08 mmol) in DMF (1.50 mL) was added TEA (0.12 g, 1.18 mmol) at room temperature. The reaction mixture was degassed with argon for three times and stirred for 2 h at 90° C. The resulting mixture was filtered and the filtrate was concentrated under reduced pressure. The residue was purified by silica gel Column chromatography, eluted with CH2Cl2/MeOH (10/1). The fractions contained desired product were combined and concentrated. The residue was purified by Prep-HPLC with the following conditions Column: XBridge Prep C18 OBD Column, 19×150 mm 5 μm; Mobile Phase A: Water (10 mmol/L NH4HCO3), Mobile Phase B: ACN; Flow rate: 20 mL/min; Gradient: 30 B to 60 B in 5.8 min; 210/254 nm; RT: 5.56 min. The fractions contained desired product were combined and concentrated to afford 3-(2-[imidazo[1,2-a]pyrimidin-6-yl]ethynyl)-1-[(3S,5R)-5-(methoxymethyl)-1-(prop-2-enoyl)pyrrolidin-3-yl]-5-(methylamino)pyrazole-4-carboxamide (36.7 mg, 20%) as a white solid. MS ESI calculated for C22H24N8O₃ [M+H]+, 449.20, found 449.15; 1H NMR (400 MHz, DMSO-d6) δ 9.37-9.36 (m, 1H), 8.66 (d, J=2.4 Hz, 1H), 7.94 (d, J=1.4 Hz, 1H), 7.91-7.76 (m, 1H), 7.48-6.80 (m, 2H), 6.77-6.47 (m, 2H), 6.17-5.96 (m, 1H), 5.69-5.43 (m, 1H), 5.25-5.23 (m, 1H), 4.63-4.34 (m, 1H), 4.11-3.71 (m, 2H), 3.67-3.38 (m, 2H), 3.30 (s, 3H), 2.96-2.93 (m, 3H), 2.71-2.54 (m, 1H), 2.44-2.24 (m, 1H).

### Example 58: 3-(2-[Imidazo[1,2-a]pyrimidin-7-yl]ethynyl)-1-[(3S,5R)-5-(methoxymethyl)-1-(prop-2-enoyl)pyrrolidin-3-yl]-5-(methylamino)pyrazole-4-carboxamide

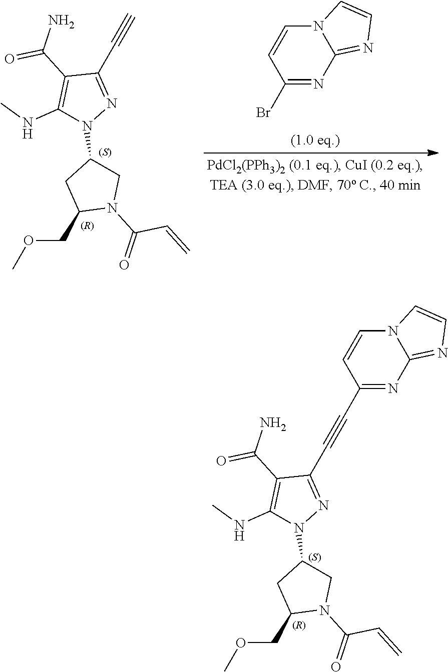

To a stirred mixture of 3-ethynyl-1-[(3S,5R)-5-(methoxymethyl)-1-(prop-2-enoyl)pyrrolidin-3-yl]-5-(methylamino)pyrazole-4-carboxamide (0.13 g, 0.39 mmol) and 7-bromoimidazo[1,2-a]pyrimidine (77.69 mg, 0.39 mmol) in DMF (2.50 mL) were added CuI (14.94 mg, 0.07 mmol), Pd(PPh₃)₂Cl₂ (27.54 mg, 0.03 mmol) and TEA (0.12 g, 1.17 mmol) at room temperature. The reaction mixture was degassed with argon for three times and stirred for 40 min at 70° C. The resulting mixture was concentrated under reduced pressure. The residue was purified by silica gel Column chromatography, eluted with MeOH in DCM (0-7%). The fractions contained desired product were combined and concentrated to afford crude product. The crude product was purified by Prep-HPLC with the following conditions Column: XBridge Shield RP18 OBD Column, 30×150 mm, 5 μm; Mobile Phase A: Water (10 mmol/L NH4HCO3), Mobile Phase B: ACN; Flow rate: 30 mL/min; Gradient: 15 B to 50 B in 6 min; 210/254 nm; RT: 5.58 min. The fractions contained desired product were combined and concentrated to afford 3-(2-[imidazo[1,2-a]pyrimidin-7-yl]ethynyl)-1-[(3S,5R)-5-(methoxymethyl)-1-(prop-2-enoyl)pyrrolidin-3-yl]-5-(methylamino)pyrazole-4-carboxamide (62.1 mg, 34%) as a light yellow solid. MS ESI calculated for C22H24N8O3 [M+H]+, 449.20, found 449.10; 1H NMR (300 MHz, DMSO-d6) δ 9.02 (d, J=7.0 Hz, 1H), 8.00 (d, J=1.4 Hz, 1H), 7.85 (s, 1H), 7.60-7.11 (m, 2H), 7.08-6.32 (m, 3H), 6.28-6.07 (m, 1H), 5.68 (d, J=10.1 Hz, 1H), 5.23-5.20 (m, 1H), 4.54 (s, 1H), 4.08-3.66 (m, 2H), 3.25 (s, 3H), 3.65-3.40 (m, 2H), 2.93-2.91 (m, 3H), 2.75-2.56 (m, 1H), 2.38-2.22 (m, 1H).

### Example 59: 3-[2-(1-Tert-butyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

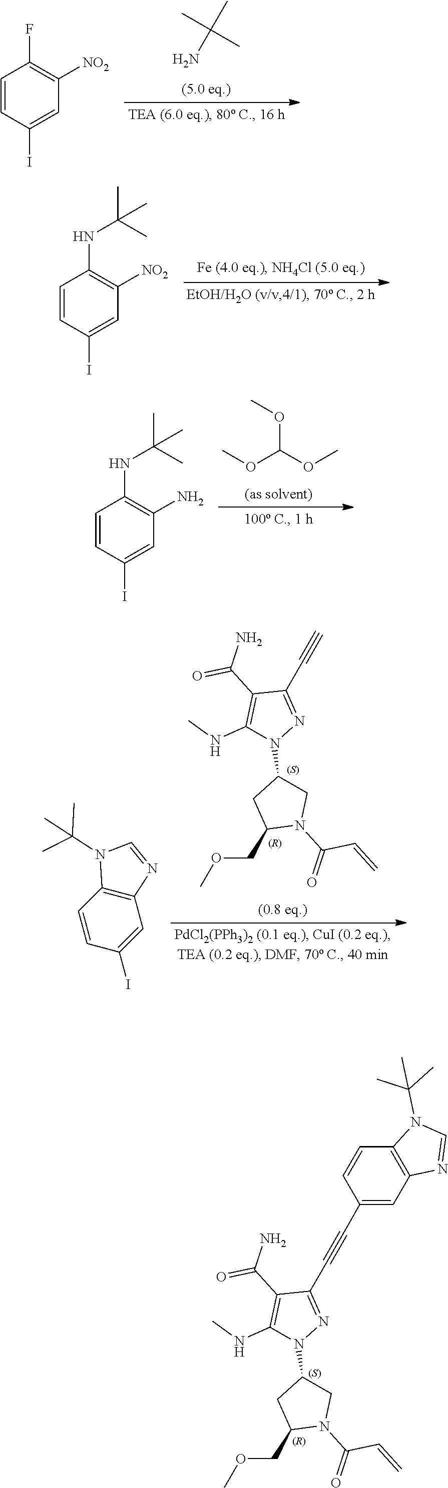

### Step 1: N-Tert-butyl-4-iodo-2-nitroaniline

To a stirred solution of 1-fluoro-4-iodo-2-nitrobenzene (2.00 g, 7.49 mmol) in Et3N (4.55 g, 44.94 mmol) was added tert-butylamine (2.74 g, 37.46 mmol) at room temperature. The reaction mixture was stirred for 16 h at 80° C. The resulting mixture was cooled down to room temperature and concentrated under reduced pressure. The residue was dissolved in water (30 mL), extracted with EtOAc (3×50 mL). The combined organic layers was dried over anhydrous Na2SO4 and filtered. The filtrate was concentrated under reduced pressure to afford N-tert-butyl-4-iodo-2-nitroaniline (2.3 g, 95%) as an off-white solid which was used in the next step directly without further purification. MS ESI calculated for C10H13IN2O2 [M+H]+, 321.00, found 321.10.

### Step 2: N1-Tert-butyl-4-iodobenzene-1,2-diamine

To a stirred mixture of N-tert-butyl-4-iodo-2-nitroaniline (2.30 g, 7.18 mmol) in EtOH (28.00 mL) and H2O (7.00 mL) were added Fe (1.60 g, 28.65 mmol) and NH4Cl (1.92 g, 35.92 mmol) at room temperature. The reaction mixture was stirred for 2 h at 70° C. The resulting mixture was filtered, the filter cake was washed with EtOH (3×70 mL). The filtrate was concentrated under reduced pressure. The residue was dissolved in water (50 mL). The resulting mixture was extracted with EtOAc (3×50 mL). The combined organic layers were washed with brine (30 mL), dried over anhydrous Na2SO4 and filtered. The filtrate was concentrated under reduced pressure to afford N1-tert-butyl-4-iodobenzene-1,2-diamine (2.1 g, crude) as a dark green solid which was used in the next step directly without further purification. MS ESI calculated for C10H15IN2[M+H]+, 291.03, found 291.05.

### Step 3: 1-Tert-butyl-5-iodo-1,3-benzodiazole

A solution of N1-tert-butyl-4-iodobenzene-1,2-diamine (1.90 g, 6.54 mmol) in trimethyl orthoformate (20.00 mL) was stirred for 1 h at 100° C. The resulting mixture was concentrated under reduced pressure. The residue was purified by silica gel Column chromatography, eluted with EA in PE (0-53%). The fractions contained desired product were combined and concentrated to afford 1-tert-butyl-5-iodo-1,3-benzodiazole (1.5 g, 76%) as a brown solid. MS ESI calculated for C11H13IN2 [M+H]+, 301.01, found 300.90.

### Step 4: 3-[2-(1-Tert-butyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a stirred mixture of 3-ethynyl-1-[(3S,5R)-5-(methoxymethyl)-1-(prop-2-enoyl)pyrrolidin-3-yl]-5-(methylamino)pyrazole-4-carboxamide (0.13 g, 0.39 mmol) and 1-tert-butyl-5-iodo-1,3-benzodiazole (0.14 g, 0.47 mmol) in DMF (2.50 mL) were added Pd(PPh₃)₂Cl₂ (27.54 mg, 0.03 mmol), CuI (14.94 mg, 0.08 mmol) and TEA (0.12 g, 1.17 mmol) at room temperature. The reaction mixture was degassed with argon for three times and stirred 40 min at 70° C. The resulting mixture was concentrated under reduced pressure. The residue was purified by silica gel Column chromatography, eluted with MeOH in DCM (0-6%) to afford crude product. The crude product was purified by reverse flash chromatography with the following conditions: Column, C18 silica gel; mobile phase, NH4HCO3 in water, 10% to 32% gradient in 10 min; detector, UV 254 nm. The fractions contained desired product were combined and concentrated to afford 3-[2-(1-tert-butyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (86.8 mg, 43%) as an off-white solid. MS ESI calculated for C27H33N7O₃ [M+H]+, 504.26, found 504.15; 1H NMR (300 MHz, DMSO) δ 8.45 (s, 1H), S 7.93-7.90 (m, 2H), 7.39 (d, J=8.6 Hz, 2H), 7.01-6.47 (m, 3H), 6.16 (d, J=16.7 Hz, 1H), 5.68 (d, J=10.4 Hz, 1H), S 5.25 (s, 1H), 4.52-4.38 (m, 1H), 4.13-3.68 (m, 2H), 3.66-3.41 (m, 2H), 3.25 (s, 3H), 2.93-2.91 (m, 3H), 2.60 (m, 1H), 2.29-2.26 (m, 1H), 1.71 (s, J=3.4 Hz, 9H).

### Example 60: 3-[2-[2-(Dimethylamino)-1-methyl-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide

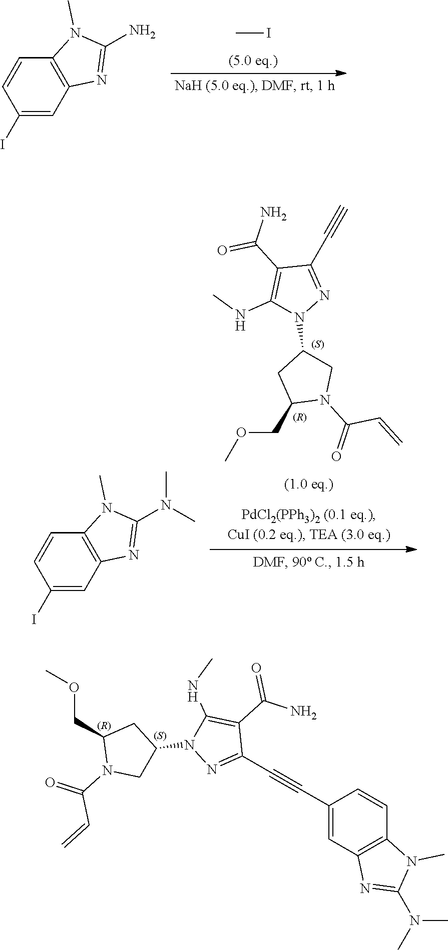

### Step 1: 5-Iodo-N,N,1-trimethyl-1,3-benzodiazol-2-amine

To a stirred solution of 5-iodo-1-methyl-1,3-benzodiazol-2-amine (0.50 g, 1.83 mmol) in DMF (5.00 mL, 64.60 mmol) was added NaH (0.18 g, 7.32 mmol) in portions at room temperature. To the above mixture was added methyl iodide (1.30 g, 9.15 mmol) dropwise at room temperature. The reaction mixture was stirred for 1 h. To the resulting mixture was added H2O (100 mL). The resulting mixture was extracted with EtOAc (5×50 mL). The combined organic layers was dried over anhydrous Na2SO4 and filtered. The filtered was concentrated under reduced pressure. The residue was purified by silica gel Column chromatography, eluted with PE/EtOAc (0-50%). The fractions contained desired product were combined and concentrated to afford 5-iodo-N,N,1-trimethyl-1,3-benzodiazol-2-amine (0.40 g, 72%) as a light yellow solid. MS ESI calculated for C10H12IN3 [M+H]+, 302.01, found 301.95.

### Step 2: 3-[2-[2-(Dimethylamino)-1-methyl-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide

To a stirred mixture of 3-ethynyl-1-[(3S,5R)-5-(methoxymethyl)-1-(prop-2-enoyl)pyrrolidin-3-yl]-5-(methylamino)pyrazole-4-carboxamide (0.13 g, 0.39 mmol) and 5-iodo-N,N,1-trimethyl-1,3-benzodiazol-2-amine (0.14 g, 0.47 mmol) in DMF (2.00 mL) were added CuI (15.00 mg, 0.08 mmol), Pd(PPh₃)₂Cl₂ (27.50 mg, 0.04 mmol) and TEA (0.12 g). The reaction mixture was degassed with argon for three times and stirred for 1.5 h at 90° C. The resulting mixture was concentrated under reduced pressure. The residue was purified by silica gel Column chromatography, eluted with PE/EtOAc (0-10%) to afford the crude. The crude was purified by reverse phase chromatography with the following conditions: Column: Spherical C18, 20-40 μm, 40 g; Mobile Phase A: Water (10 mmol/L NH₄HCO₃); Mobile Phase B: ACN; Flow rate: 40 mL/min; 5%-60% within 60 min, Detector: UV 254 & 220 nm. The fractions contained desired product were combined and concentrated to afford 3-[2-[2-(dimethylamino)-1-methyl-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide (41.3 mg, 20%) as a white solid. MS ESI calculated for C₂₆H₃₂N₈O₃ [M+H]⁺, 505.26, found 505.15; ¹H NMR (300 MHz, DMSO-d₆) δ 7.54 (d, J=1.5 Hz, 1H), 7.38 (d, J=8.2 Hz, 2H), 7.26 (d, J=8.3 Hz, 1H), 6.85-6.50 (m, 3H), 6.18 (d, J=16.7 Hz, 1H), 5.70 (d, J=10.5 Hz, 1H), 5.26 (d, J=7.9 Hz, 1H), 4.53-4.38 (m, 1H), 4.10-3.69 (m, 2H), 3.66 (s, 3H), 3.54-3.41 (m, 2H), 3.33-3.30 (m, 3H), 2.97 (s, 9H), 2.60-2.58 (m, 1H), 2.30-2.28 (m, 1H).

### Example 61: 3-[2-(2-Cyclopropyl-4,6-difluoro-1H-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

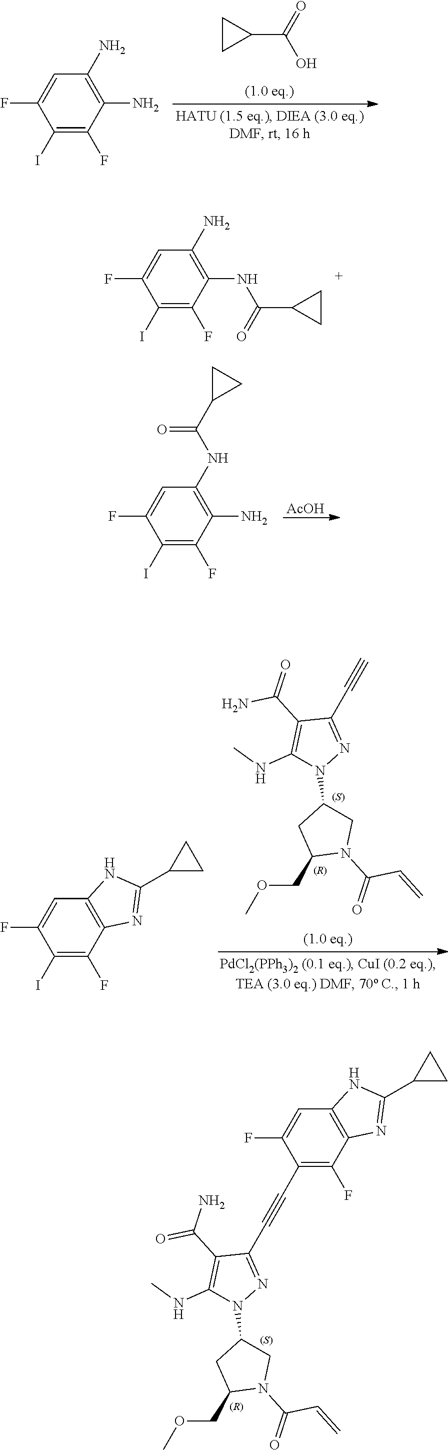

### Step 1: N-(6-Amino-2,4-difluoro-3-iodophenyl)cyclopropanecarboxamide and N-(2-amino-3,5-difluoro-4-iodophenyl)cyclopropanecarboxamide

To a solution of cyclopropanecarboxylic acid (1.91 g, 22.22 mmol) in DMF (30.00 mL) were added HATU (10.56 g, 27.77 mmol) and DIEA (7.18 g, 55.55 mmol) at room temperature. The reaction mixture was stirred for 30 min at room temperature. To the above mixture was added 3,5-difluoro-4-iodobenzene-1,2-diamine (5.00 g, 18.51 mmol) in DMF (20.00 mL). The resulting mixture was stirred for 16 h at room temperature. The resulting mixture was diluted with EA (100 mL), washed with water (3×50 mL). The combined organic layers were washed with brine (3×50 mL), dried over anhydrous Na₂SO₄ and filtered. The filtrate was concentrated under reduced pressure. The residue was purified by silica gel Column chromatography, eluted with EA in PE (0-20%). The fractions contained desired product were combined and concentrated to afford the mixture of N-(6-amino-2,4-difluoro-3-iodophenyl)cyclopropanecarboxamide and N-(2-amino-3,5-difluoro-4-iodophenyl)cyclopropanecarboxamide (4.0 g, 63%) as a yellow solid. MS ESI calculated for C₁₀H₉F₂IN₂O [M+H]⁺, 338.97, found 339.00.

### Step 2: 2-Cyclopropyl-4,6-difluoro-5-iodo-1H-1,3-benzodiazole

A solution of N-(6-amino-2,4-difluoro-3-iodophenyl)cyclopropanecarboxamide and N-(2-amino-3,5-difluoro-4-iodophenyl)cyclopropanecarboxamide (5.00 g, 14.78 mmol) in AcOH (60.00 mL) was stirred for 24 h at 70° C. The resulting mixture was cooled down to room temperature and concentrated under reduced pressure. The residue was basified to PH 8 with saturated NaHCO₃ (aq.). The resulting mixture was diluted with water (50 mL), extracted with EA (3×100 mL). The combined organic layers were washed with brine (3×50 mL), dried over anhydrous Na2SO4 and filtered. The filtrate was concentrated under reduced pressure. The residue was purified by silica gel Column chromatography, eluted with EA in PE (0-52%). The fractions contained desired product were combined and concentrated to afford 2-cyclopropyl-4,6-difluoro-5-iodo-1H-1,3-benzodiazole (2.2 g, 46%) as a yellow solid. MS ESI calculated for C10H7F2IN2 [M+H]+, 320.96, found. 320.85.

### Step 3: 3-[2-(2-Cyclopropyl-4,6-difluoro-1H-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a stirred mixture of 2-cyclopropyl-4,6-difluoro-5-iodo-1H-1,3-benzodiazole (0.12 g, 0.38 mmol), 3-ethynyl-1-[(3S,5R)-5-(methoxymethyl)-1-(prop-2-enoyl)pyrrolidin-3-yl]-5-(methylamino)pyrazole-4-carboxamide (0.12 g, 0.38 mmol), Pd(PPh₃)₂Cl₂ (26.31 mg, 0.04 mmol) and CuI (14.28 mg, 0.08 mmol) in DMF (4.00 mL) was added TEA (0.13 g, 1.13 mmol) at room temperature. The reaction mixture was degassed with argon for three times and stirred for 1 h at 70° C. The resulting mixture was concentrated under reduced pressure. The residue was purified by silica gel Column chromatography, eluted with MeOH in CH₂Cl₂ (0-5%) to afford the crude product. The crude product was purified by reverse phase flash with the following conditions: Column: Spherical C18, 20˜40 μm, 330 g; Mobile Phase A: Water (10 mmol/L NH₄HCO₃), Mobile Phase B: ACN; Flow rate: 65 mL/min; Gradient: 30%˜40%, 20 min; Detector: 254 nm; RT: 20 min. The fractions contained desired product were combined and concentrated to afford 3-[2-(2-cyclopropyl-4,6-difluoro-1H-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (0.11 g, 55%) as an off-white solid. MS ESI calculated for C₂₆H₂₇F₂N₇O₃[M+H]⁺, 524.22, found. 524.15; ¹H NMR (300 MHz, DMSO-d₆) δ 7.57 (s, 1H), 7.28 (d, J=9.2 Hz, 1H), 6.76-6.56 (m, 2H), 6.19-6.12 (m, 1H), 5.70-5.64 (m, 1H), 5.27-5.25 (m, 1H), 4.45-4.40 (m, 1H), 4.05-4.02 (m, 1H), 3.93-3.81 (m, 1H), 3.76-3.73 (m, 1H), 3.62-3.59 (m, 1H), 3.48-3.45 (m, 3H), 2.95 (t, J=3.0 Hz, 4H), 2.31-2.30 (m, 1H), 2.29-2.27 (m, 1H), 2.14-2.12 (m, 1H), 1.08-1.04 (m, 4H).

### Example 62: 3-[2-(2-Cyclopropyl-6-fluoro-1-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

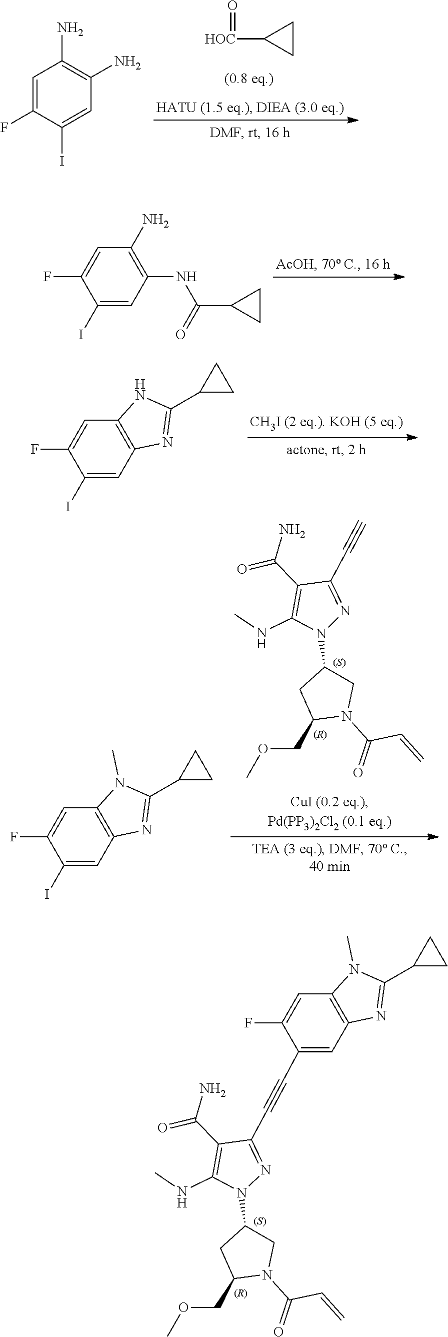

### Step 1: N-(2-Amino-4-fluoro-5-iodophenyl)cyclopropanecarboxamide

To a stirred mixture of 4-fluoro-5-iodobenzene-1,2-diamine (2.00 g, 7.94 mmol), cyclopropanecarboxylic acid (0.34 g, 3.97 mmol) and HATU (4.53 g, 11.90 mmol) in DMF (20.00 mL) was added DIEA (3.30 mL). The reaction mixture was stirred for 16 h at room temperature under nitrogen atmosphere. The resulting mixture was diluted with water (80 mL), extracted with EtOAc (3×100 mL). The combined organic layers were washed with brine (150 mL), dried over anhydrous Na2SO4 and filtered. The filtrate was concentrated under reduced pressure. The residue was purified by silica gel Column chromatography, eluted with PE/EtOAc (5/1). The fractions contained desired product were combined and concentrated to afford N-(2-amino-4-fluoro-5-iodophenyl)cyclopropanecarboxamide (1.3 g, 51%) as a white solid. MS ESI calculated for C10H10FIN2O [M+H]+, 320.98, found 321.00.

### Step 2: 2-Cyclopropyl-6-fluoro-5-iodo-1H-benzo[d]imidazole

A mixture of N-(2-amino-4-fluoro-5-iodophenyl)cyclopropanecarboxamide (1.10 g) in AcOH (20 mL) was stirred for 16 h at 70° C. under nitrogen atmosphere. The resulting mixture was concentrated under reduced pressure. The residue was basified to pH 8 with saturated NaHCO₃. The resulting mixture was extracted with EtOAc (3×100 mL). The combined organic layers were washed with brine (3×50 mL), dried over anhydrous Na2SO4 and filtered. The filtrate was concentrated under reduced pressure. The residue was purified by silica gel Column chromatography, eluted with PE/EtOAc (1/1). The fractions contained desired product were combined and concentrated to afford 2-cyclopropyl-6-fluoro-5-iodo-1H-benzo[d]imidazole as a white solid. MS ESI calculated for C10H8FIN2 [M+H]+, 302.97, found 303.05.

### Step 3: 2-Cyclopropyl-6-fluoro-5-iodo-1-methyl-1H-benzo[d]imidazole

To a stirred mixture of 2-cyclopropyl-6-fluoro-5-iodo-1H-benzo[d]imidazole (0.67 g, 2.22 mmol) and KOH (0.62 g, 11.09 mmol) in acetone (10.00 mL) was added CH3I (0.63 g, 4.44 mmol) dropwise at 0° C. under nitrogen atmosphere. The reaction mixture was stirred for 2 h at room temperature under nitrogen atmosphere. The resulting mixture was diluted with water (150 mL) and extracted with EtOAc (3×100 mL). The combined organic layers were washed with brine (3×50 mL), dried over anhydrous Na2SO4 and filtered. The filtrate was concentrated under reduced pressure. The residue was purified by silica gel Column chromatography, eluted with CH2Cl2/MeOH (10/1). The fractions contained desired product were combined and concentrated. The residue was purified by Prep-achiral-SFC with the following conditions Column: EnantioPak A1-5, 2.12×25 cm, 5 μm; Mobile Phase A: CO2, Mobile Phase B: MeOH (0.5% 2 M NH₃-MeOH); Flow rate: 40 mL/min; Gradient: 35% B; 254 nm; RT1: 4.45 min; RT2: 5.09 min; Injection volume: 1 mL; Number of runs: 28. The fractions contained desired product were combined and concentrated to afford 2-cyclopropyl-6-fluoro-5-iodo-1-methyl-1H-benzo[d]imidazole (0.54 g, 32%) as an off-white solid. MS ESI calculated for C11H10FIN2 [M+H]+, 316.99, found 316.95.

### Step 4: 3-[2-(2-Cyclopropyl-6-fluoro-1-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a stirred mixture of 2-cyclopropyl-6-fluoro-5-iodo-1-methyl-1H-benzo[d]imidazole (47.70 mg, 0.15 mmol), 3-ethynyl-1-[(3S,5R)-5-(methoxymethyl)-1-(prop-2-enoyl)pyrrolidin-3-yl]-5-(methylamino)pyrazole-4-carboxamide (50 mg, 0.15 mmol), Pd(PPh₃)₂Cl₂ (27.54 mg, 0.039 mmol) and CuI (5.75 mg, 0.03 mmol) in DMF (1.50 mL) was added TEA (0.06 mL, 0.62 mmol) dropwise at room temperature. The reaction mixture was degassed with argon for three times and stirred for 40 min at 70° C. The resulting mixture was diluted with water (50 mL), extracted with EtOAc (5×50 mL). The combined organic layers were washed with brine (3×30 mL), dried over anhydrous Na2SO4 and filtered. The filtrate was concentrated under reduced pressure. The residue was purified by silica gel Column chromatography, eluted with CH2Cl2/MeOH (10/1). The fractions contained desired product were combined and concentrated. The residue was purified by Prep-HPLC with the following conditions: Column: XBridge Prep C18 OBD Column, 19×150 mm 5 μm; Mobile Phase A: Water (10 mmol/L NH4HCO3), Mobile Phase B: ACN; Flow rate: 20 mL/min; Gradient: 30 B to 70 B in 5.8 min; 210/254 nm. The fractions contained desired product were combined and concentrated to afford the crude product. The crude product was purified by Prep-HPLC with the following conditions Column: XBridge Prep C18 OBD Column, 19×150 mm 5 μm; Mobile Phase A: Water (10 mmol/L NH4HCO3), Mobile Phase B: ACN; Flow rate: 20 mL/min; Gradient: 30 B to 70 B in 6 min; 210/254 nm. The fractions contained desired product were combined and concentrated to afford 3-[2-(2-cyclopropyl-6-fluoro-1-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (9.1 mg, 11%) as a white solid. MS ESI calculated for C27H30FN7O3 [M+H]+, 520.24, found 520.25; 1H NMR (300 MHz, DMSO) δ 7.73 (d, J=6.3 Hz, 1H), 7.61 (d, J=9.8 Hz, 1H), 7.50 (s, 1H), 6.88-6.48 (m, 2H), 6.17 (d, J=16.6 Hz, 1H), 5.90-5.58 (m, 1H), 5.27 (s, 1H), 4.49-4.46 (m, 1H), 4.03-4.01 (m, 1H), 3.91-3.70 (m, 4H), 3.63-3.59 (m, 1H), 3.52-3.38 (m, 2H), 3.31 (s, 3H), 3.00-2.86 (m, 3H), 2.69-2.67 (m, 1H), 2.29-2.25 (m, 2H), 1.26-0.95 (m, 5H).

### Example 63: 3-(2-[2-Cyclopropyl-3-methylimidazo[4,5-c]pyridin-6-yl]ethynyl)-1-[(3S,5R)-5-(methoxymethyl)-1-(prop-2-enoyl)pyrrolidin-3-yl]-5-(methylamino)pyrazole-4-carboxamide

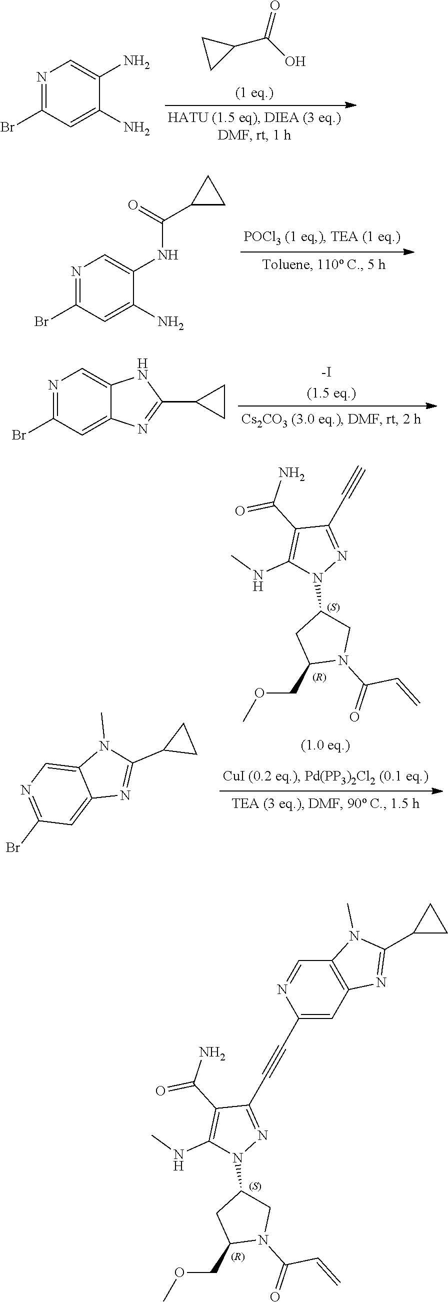

### Step 1: N-(5-Amino-2-bromopyridin-4-yl)cyclopropanecarboxamide

To a stirred mixture of 6-bromopyridine-3,4-diamine (1.00 g, 5.31 mmol) and HATU (3.03 g, 7.98 mmol) in DMF (10.00 mL) was added DIEA (2.06 g, 15.96 mmol) dropwise at room temperature. The reaction mixture was stirred for 0.5 h. To the above mixture was added cyclopropanecarboxylic acid (0.46 g, 5.32 mmol) dropwise over 3 min. The reaction mixture was stirred for additional 1 h at room temperature. The resulting mixture was quenched by the addition of water (10 mL) at room temperature and extracted with EtOAc (3×20 mL). The combined organic layers were washed with brine (3×30 mL), dried over anhydrous Na2SO4 and filtered. The filtrate was concentrated under reduced pressure. The residue was purified by silica gel column chromatography, eluted with PE/EtOAc (1/1). The fractions contained desired product were combined and concentrated to afford N-(5-amino-2-bromopyridin-4-yl)cyclopropanecarboxamide (0.96 g, 70%) as a light yellow solid. MS ESI calculated for C9H10BrN30 [M+H]+, 256.00, 258.00, found 256.05, 258.05.

### Step 2: 6-Bromo-2-cyclopropyl-3H-imidazo[4,5-c]pyridine

To a stirred solution of N-(5-amino-2-bromopyridin-4-yl)cyclopropanecarboxamide (1.20 g, 4.69 mmol) and TEA (0.47 g, 4.69 mmol) in toluene (12.00 mL) was added phosphorus oxychloride (0.72 g, 4.69 mmol) dropwise at room temperature. The reaction mixture was stirred for 5 h at 110° C. The resulting mixture was cooled down to room temperature and concentrated under reduced pressure. The residue was purified by silica gel Column chromatography, eluted with PE/EtOAc (1/3). The fractions contained desired product were combined and concentrated to afford 6-bromo-2-cyclopropyl-3H-imidazo[4,5-c]pyridine (0.71 g, 63%) as a light yellow solid. MS ESI calculated for C9H8BrN3 [M+H]+, 237.99, 239.99, found 238.05, 240.05.

### Step 3: 6-Bromo-2-cyclopropyl-3-methylimidazo[4,5-c]pyridine

To a stirred mixture of 6-bromo-2-cyclopropyl-3H-imidazo[4,5-c]pyridine (0.71 g, 2.98 mmol) and Cs2CO3 (2.91 g, 8.94 mmol) in DMF (7.00 mL) was added methyl iodide (0.63 g, 4.47 mmol) dropwise at room temperature. The reaction mixture was stirred for 2 h at room temperature. The resulting mixture was filtered, the filter cake was washed with DMF (2 mL). the filtrate was purified by reverse flash chromatography with the following conditions: Column, C18 silica gel; mobile phase, ACN in water, 5% to 70% gradient in 30 min; detector, UV 254 nm. The fractions contained desired product were combined and concentrated to afford 6-bromo-2-cyclopropyl-3-methylimidazo[4,5-c]pyridine (0.13 g, 17%) as an off-white solid. MS ESI calculated for C10H10BrN3 [M+H]+, 252.01, 254.00, found 252.10, 254.10.

### Step 4: 3-(2-[2-Cyclopropyl-3-methylimidazo[4,5-c]pyridin-6-yl]ethynyl)-1-[(3S,5R)-5-(methoxymethyl)-1-(prop-2-enoyl)pyrrolidin-3-yl]-5-(methylamino)pyrazole-4-carboxamide

To a stirred mixture of 3-ethynyl-1-[(3S,5R)-5-(methoxymethyl)-1-(prop-2-enoyl)pyrrolidin-3-yl]-5-(methylamino) pyrazole-4-carboxamide (0.15 g, 0.43 mmol), 6-bromo-2-cyclopropyl-3-methylimidazo[4,5-c]pyridine (0.11 g, 0.44 mmol), Pd(PPh3)2Cl2 (30.71 mg, 0.04 mmol) and CuI (16.67 mg, 0.08 mmol) in DMF (2.00 mL) was added TEA (0.13 g, 1.31 mmol) at room temperature. The reaction mixture was degassed with argon for three times and stirred for 1.5 h at 90° C. The resulting mixture was cooled down to room temperature and concentrated under reduced pressure. The residue was purified by silica gel Column chromatography, eluted with PE/EtOAc (1/1). The fractions contained desired product were combined and concentrated, the residue was purified by Prep-HPLC with the following conditions Column: SunFire Prep C18 OBD Column, 19×150 mm 5 μm 10 nm; Mobile Phase A: water (0.1% FA), Mobile Phase B: MeOH; Flow rate: 20 mL/min; Gradient: 15 B to 35 B in 6 min; 210/254 nm; RT: 5.56 min. The fractions contained desired product were combined and concentrated to afford 3-(2-[2-cyclopropyl-3-methylimidazo[4,5-c]pyridin-6-yl]ethynyl)-1-[(3S,5R)-5-(methoxymethyl)-1-(prop-2-enoyl)pyrrolidin-3-yl]-5(methylamino)pyrazole-4-carboxamide (49.8 mg, 22%) as an off-white solid. MS ESI calculated for C26H30N8O3 [M+H]+, 503.24, found 503.20; 1H NMR (300 MHz, DMSO-d6) δ 8.88 (d, J=1.0 Hz, 1H), 7.81 (s, 1H), 7.46 (s, 1H), 6.86-6.52 (m, 3H), 6.17 (d, J=16.6 Hz, 1H), 5.70 (d, J=10.4 Hz, 1H), 5.27 (dd, J=13.6, 6.8 Hz, 1H), 4.40-4.05 (m, 1H), 3.98-3.96 (m, 4H), 3.92-3.82 (m, 1H), 3.79-3.56 (m, 1H), 3.53-3.41 (m, 2H), 3.31 (s, 3H), 2.98-2.95 (m, 3H), 2.36-2.29 (m, 2H), 1.27-1.01 (m, 4H).

### Example 64: 1-[(3S,5R)-5-(Methoxymethyl)-1-(prop-2-enoyl) pyrrolidin-3-yl]-5-(methylamino)-3-[2-(quinoxalin-6-yl) ethynyl]pyrazole-4-carboxamide

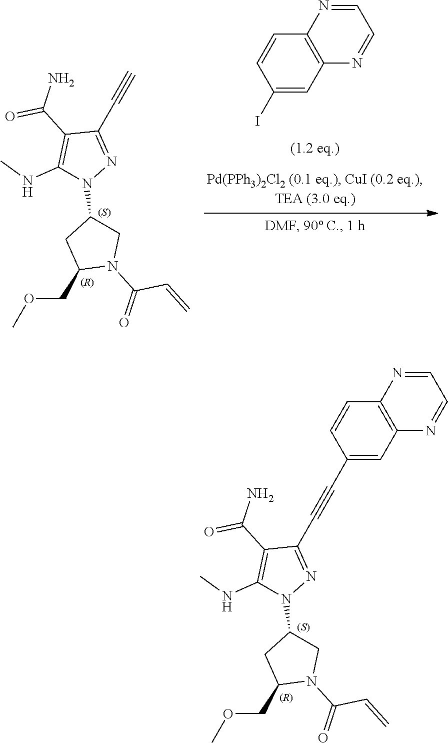

To a mixture of 6-iodoquinoxaline (0.12 g, 0.47 mmol), 3-ethynyl-1-[(3S,5R)-5-(methoxymethyl)-1-(prop-2-enoyl)pyrrolidin-3-yl]-5-(methylamino) pyrazole-4-carboxamide (0.13 g, 0.39 mmol), CuI (14.94 mg, 0.08 mmol) and Pd(PPh₃)₂Cl₂ (27.54 mg, 0.04 mmol) in DMF (2.00 mL) was added TEA (0.12 g, 1.18 mmol). The reaction mixture was degassed with argon for three times and stirred for 1 h at 90° C. The resulting mixture was concentrated under reduced pressure. The residue was purified by reverse phase chromatography with the following conditions: Column: Spherical C18, 20-40 μm, 40 g; Mobile Phase A: Water (10 mmol/L NH₄HCO₃), Mobile Phase B: ACN; Flow rate: 40 mL/min; Gradient: 20%-40% within 40 min, Detector: UV 254/220 nm; RT: 30 min. The fractions contained desired product were combined and concentrated to afford 1-[(3S,5R)-5-(methoxymethyl)-1-(prop-2-enoyl) pyrrolidin-3-yl]-5-(methylamino)-3-[2-(quinoxalin-6-yl) ethynyl]pyrazole-4-carboxamide (91.4 mg, 51%) as an off-white solid. MS ESI calculated for C₂₄H₂₅N₇O₃ [M+H]⁺, 460.20, found 460.15; ¹H NMR (300 MHz, DMSO-d₆) δ 9.04-8.98 (m, 2H), 8.34 (d, J=1.7 Hz, 1H), 8.16 (d, J=8.7 Hz, 1H), 7.98 (dd, J=8.7, 1.9 Hz, 1H), 7.99-6.93 (m, 2H), 6.79-6.47 (m, 2H), 6.21-6.18 (m, 1H), 5.73-5.68 (m, 1H), 5.32-5.20 (m, 1H), 4.51-4.48 (m, 1H), 4.09-3.70 (m, 2H), 3.64-3.44 (m, 2H), 3.31 (s, 3H), 2.95-2.92 (m, 3H), 2.67-2.58 (m, 1H), 2.39-2.23 (m, 1H).

### Example 65: 1-((3S,5R)-1-Acryloyl-5-(methoxymethyl)pyrrolidin-3-yl)-5-(methylamino)-3-((3-methylquinolin-7-yl)ethynyl)-1H-pyrazole-4-carboxamide

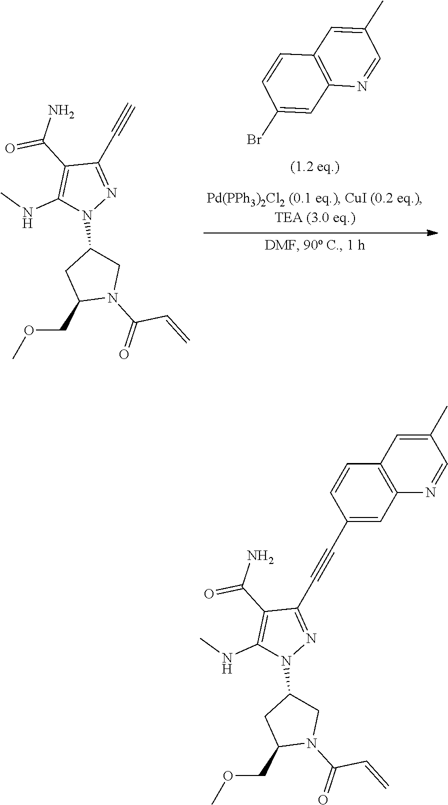

To a stirred mixture of 5-amino-3-ethynyl-1-[(3S,5R)-5-(methoxymethyl)-1-(prop-2-enoyl)pyrrolidin-3-yl]pyrazole-4-carboxamide (0.13 g, 0.41 mmol), 7-bromo-3-methylquinoline (0.11 g, 0.49 mmol), CuI (15.60 mg, 0.08 mmol) and Pd(PPh₃)₂Cl₂ (28.75 mg, 0.04 mmol) in DMF (1.50 mL) was added TEA (0.17 mL, 1.22 mmol). The reaction mixture was degassed with argon for three times and stirred for 1 h at 90° C. The resulting mixture was diluted with water (20 mL), extracted with EA (3×20 mL). The combined organic layers were washed with brine (3×20 mL), dried over anhydrous Na₂SO₄ and filtered. The filtrate was concentrated under reduced pressure. The residue was purified by Prep-HPLC with the following conditions: Column: SunFire Prep C18 OBD Column, 19×150 mm 5 μm 10 nm; Mobile Phase A: Water (0.1% FA), Mobile Phase B: MeOH; Flow rate: 20 mL/min; Gradient: 50 B to 60 B in 6 min; 210/254 nm; RT: 5.56 min. The fractions contained desired product were combined and concentrated to afford 1-((3S,5R)-1-acryloyl-5-(methoxymethyl)pyrrolidin-3-yl)-5-(methylamino)-3-((3-methylquinolin-7-yl)ethynyl)-1H-pyrazole-4-carboxamide (30.3 mg, 16%) as a yellow solid. MS ESI calculated for C₂₆H₂₈N₆O₃[M+H]⁺, 473.22, found 473.35; ¹H NMR (400 MHz, DMSO-d₆) δ 8.87 (s, 1H), 8.23-8.19 (m, 2H), 7.99 (d, J=8.5 Hz, 1H), 7.71 (d, J=8.4 Hz, 1H), 7.37 (s, 1H), 6.90 (s, 1H), 6.76-6.69 (m, 1H), 6.63-6.56 (m, 1H), 6.20-6.14 (m, 1H), 5.72-5.68 (m, 1H), 5.30-5.19 (m, 1H), 4.66-4.30 (m, 1H), 4.13-3.80 (m, 2H), 3.76-3.68 (m, 1H), 3.62-3.58 (m, 1H), 3.52-3.47 (m, 3H), 3.30 (s, 3H), 2.94 (s, 3H), 2.70-2.52 (m, 1H), 2.34-2.25 (m, 1H).

### Example 66: 3-(2-[Imidazo[1,2-b]pyridazin-7-yl]ethynyl)-1-[(3S,5R)-5-(methoxymethyl)-1-(prop-2-enoyl)pyrrolidin-3-yl]-5-(methylamino)pyrazole-4-carboxamide

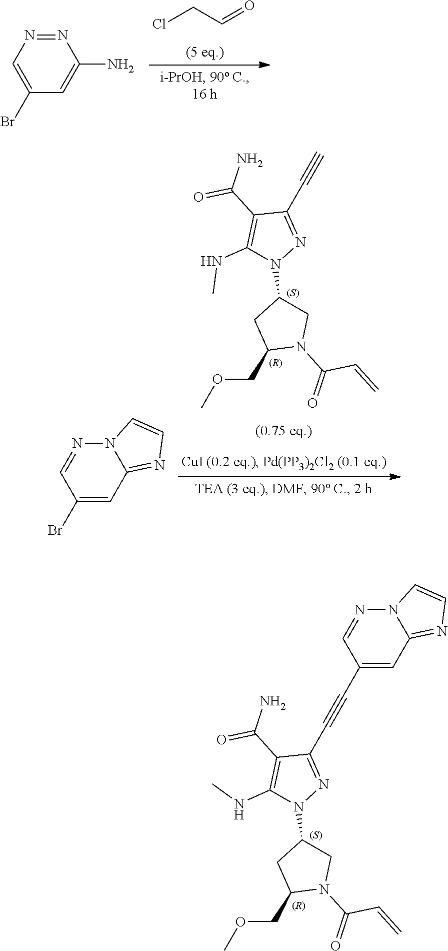

### Step 1: 7-Bromoimidazo[1,2-b]pyridazine

To a stirred solution of 5-bromopyridazin-3-amine (0.5 g, 2.87 mmol) in i-PrOH (5.50 mL) was added chloroacetaldehyde (2.37 mL, 12.07 mmol, 40%) dropwise at room temperature under argon atmosphere. The reaction mixture was stirred for 16 h at 90° C. under argon atmosphere. The resulting mixture was diluted with water (100 mL) and extracted with EtOAc (3×150 mL). The combined organic layers were washed with brine (200 mL), dried over anhydrous Na₂SO₄ and filtered. The filtrate was concentrated under reduced pressure. The residue was purified by silica gel Column chromatography, eluted with CH₂Cl₂/MeOH (10/1). The fractions contained desired product were combined and concentrated to afford 7-bromoimidazo[1,2-b]pyridazine (0.44 g, 78%) as a white solid. MS ESI calculated for C₆H₄BrN₃ [M+H]⁺, 197.96, found 198.00.

### Step 2: 3-(2-[Imidazo[1,2-b]pyridazin-7-yl]ethynyl)-1-[(3S,5R)-5-(methoxymethyl)-1-(prop-2-enoyl)pyrrolidin-3-yl]-5-(methylamino)pyrazole-4-carboxamide

To a stirred mixture of 3-ethynyl-1-[(3S,5R)-5-(methoxymethyl)-1-(prop-2-enoyl)pyrrolidin-3-yl]-5-(methylamino)pyrazole-4-carboxamide (0.17 g, 0.51 mmol), 7-bromoimidazo[1,2-b]pyridazine (0.15 g, 0.77 mmol), Pd(PPh₃)₂Cl₂ (36.01 mg, 0.05 mmol) and CuI (19.54 mg, 0.10 mmol) in DMF (2.00 mL) was added TEA (0.21 mL, 2.11 mmol) at room temperature. The reaction mixture was degassed with argon for three times and stirred for 2 h at 90° C. The resulting mixture was diluted with water (30 mL) and extracted with EtOAc (3×30 mL). The combined organic layers were washed with brine (100 mL), dried over anhydrous Na₂SO₄ and filtered. The filtrate was concentrated under reduced pressure. The residue was purified by silica gel Column chromatography, eluted with CH₂Cl₂/MeOH (10/1). The fractions contained desired product were combined and concentrated. The residue was purified by Prep-HPLC with the following conditions Column: Atlantis Prep T3 OBD Column, 19×250 mm 10μ; Mobile Phase A: Water (0.1% FA), Mobile Phase B: ACN; Flow rate: 20 mL/min; Gradient: 25 B to 55 B in 6 min; 210/254 nm; RT: 5.56 min. The fractions contained desired product were combined and concentrated to afford 3-(2-[imidazo[1,2-b]pyridazin-7-yl]ethynyl)-1-[(3S,5R)-5-(methoxymethyl)-1-(prop-2-enoyl)pyrrolidin-3-yl]-5-(methylamino)pyrazole-4-carboxamide (37.7 mg, 16%) as a yellow solid. MS ESI calculated for C₂₂H₂₄N₈O₃ [M+H]⁺, 449.20, found 449.20; ¹H NMR (400 MHz, DMSO-d₆) δ 8.67 (d, J=2.0 Hz, 1H), 8.52-8.33 (m, 2H), 7.92 (d, J=1.2 Hz, 1H), 7.30 (s, 1H), 6.90 (s, 1H), 6.82-6.45 (m, 2H), 6.17-5.96 (m, 1H), 5.69-5.43 (m, 1H), 5.25-5.23 (m, 1H), 4.63-4.32 (m, 1H), 4.17-3.70 (m, 2H), 3.68-3.41 (m, 2H), 3.30 (d, J=5.5 Hz, 3H), 2.94-2.76 (m, 3H), 2.65-2.60 (m, 1H), 2.41-2.20 (m, 1H).

### Example 67: 1-[(3S,5R)-5-(Methoxymethyl)-1-(prop-2-enoyl)pyrrolidin-3-yl]-5-(methylamino)-3-(2-[pyrazolo[1,5-a]pyridin-6-yl]ethynyl)pyrazole-4-carboxamide

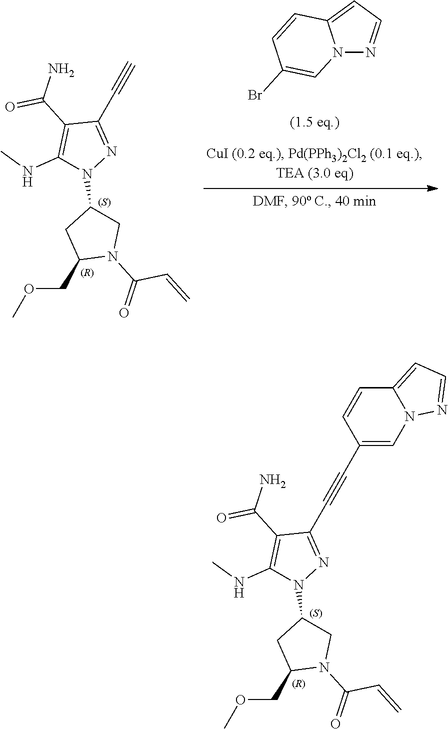

To a stirred mixture of 5-amino-3-ethynyl-1-[(3S,5R)-5-(methoxymethyl)-1-(prop-2-enoyl)pyrrolidin-3-yl]pyrazole-4-carboxamide (0.11 g, 0.35 mmol), 6-bromopyrazolo[1,5-a]pyridine (0.10 g, 0.52 mmol), Pd(PPh₃)₂Cl₂ (24.33 mg, 0.03 mmol) and CuI (13.20 mg, 0.07 mmol) in DMF (1.50 mL) was added TEA (0.14 mL, 1.01 mmol). The reaction mixture was degassed with argon for three times and stirred for 40 min at 90° C. The resulting mixture was concentrated under reduced pressure. The residue was purified by silica gel Column chromatography, eluted with 6% MeOH in DCM. The fractions contained desired product were combined and concentrated. The crude product was purified by Prep-HPLC with the following conditions Column: XBridge Prep C18 OBD Column, 19×150 mm 5 μm; Mobile Phase A: Water (10 mmol/L NH₄HCO₃), Mobile Phase B: ACN; Flow rate: 20 mL/min; Gradient: 25 B to 55 B in 6 min; 210/254 nm. The fractions contained desired product were combined and concentrated to afford 1-[(3S,5R)-5-(methoxymethyl)-1-(prop-2-enoyl)pyrrolidin-3-yl]-5-(methylamino)-3-(2-[pyrazolo[1,5-a]pyridin-6-yl]ethynyl)pyrazole-4-carboxamide (31.9 mg, 20%) as an off-white solid. MS ESI calculated for C₂₃H₂₅N₇O₃ [M+H]⁺, 448.20, found 448.20; ¹H NMR (400 MHz, DMSO-d₆): δ 9.14 (s, 1H), 8.12 (d, J=2.2 Hz, 1H), 7.78-7.70 (m, 1H), 7.30 (dd, J=9.2, 1.5 Hz, 2H), 6.88-6.48 (m, 4H), 6.22-6.13 (m, 1H), 5.73-5.68 (m, 1H), 5.26-5.22 (m, 1H), 4.55-4.38 (m, 1H), 4.10-3.68 (m, 2H), 3.64-3.39 (m, 2H), 3.33-3.30 (m, 3H), 2.95-2.94 (m, 3H), 2.53-2.51 (m, 1H), 2.39-2.35 (m, 1H).

### Example 68: 1-((3S,5R)-1-Acryloyl-5-(methoxymethyl)pyrrolidin-3-yl)-3-((1-ethyl-4-fluoro-1H-benzo[d]imidazol-5-yl)ethynyl)-5-(methylamino)-1H-pyrazole-4-carboxamide

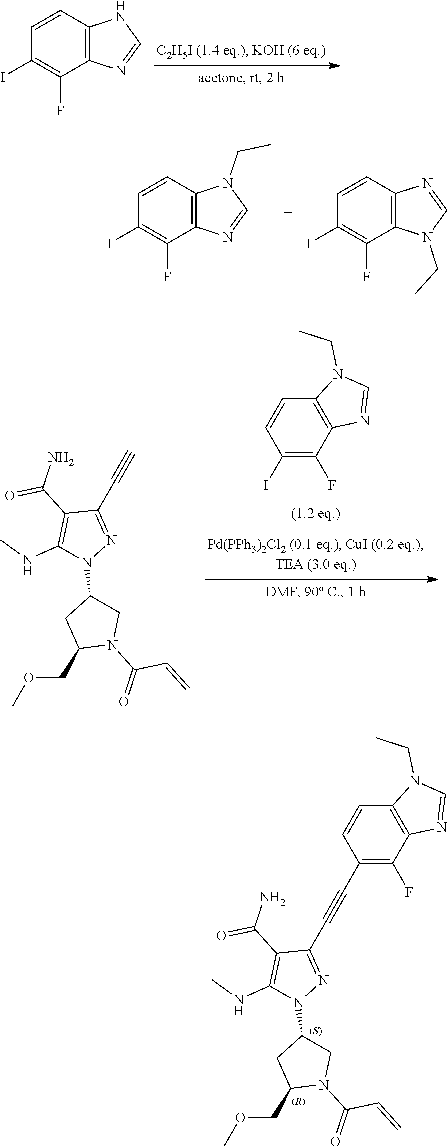

### Step 1: 1-Ethyl-4-fluoro-5-iodo-1,3-benzodiazole and 1-ethyl-7-fluoro-6-iodo-1,3-benzodiazole

To a stirred mixture of 4-fluoro-5-iodo-1H-1,3-benzodiazole (2.00 g, 7.63 mmol) and KOH (2.57 g, 45.80 mmol) in acetone (20.00 mL) was added iodoethane (1.67 g, 10.69 mmol) at room temperature. The reaction mixture was stirred for 2 h at room temperature. The resulting mixture was diluted with water (50 mL) and extracted with EA (3×70 mL). The combined organic layers were washed with brine (2×50 mL), dried over anhydrous Na₂SO₄ and filtered. The filtrate was concentrated under reduced pressure. The residue was purified by reverse flash chromatography with the following conditions: Column, C18 silica gel; mobile phase, ACN in water (10 mmol/L NH₄HCO₃), 25% to 55% gradient in 30 min; detector, UV 254 nm. The fraction contained desired product were combined and concentrated to afford 1-ethyl-4-fluoro-5-iodo-1,3-benzodiazole (0.80 g, 36%) as a yellow solid. MS ESI calculated for C₉H₈FIN₂ [M+H]⁺, 290.97, found 290.99; And also afford 1-ethyl-7-fluoro-6-iodo-1,3-benzodiazole (0.78 g, 35%) as a yellow solid. MS ESI calculated for C₉H₈FIN₂ [M+H]⁺, 290.97, found 290.95.

### Step 2: 1-((3S,5R)-1-Acryloyl-5-(methoxymethyl)pyrrolidin-3-yl)-3-((1-ethyl-4-fluoro-1H-benzo[d]imidazol-5-yl)ethynyl)-5-(methylamino)-1H-pyrazole-4-carboxamide

To a stirred mixture of 3-ethynyl-1-[(3S,5R)-5-(methoxymethyl)-1-(prop-2-enoyl)pyrrolidin-3-yl]-5-(methylamino)pyrazole-4-carboxamide (0.2 g, 0.60 mmol), 1-ethyl-4-fluoro-5-iodo-1,3-benzodiazole (0.21 g, 0.72 mmol), CuI (22.99 mg, 0.12 mmol) and Pd(PPh₃)₂Cl₂ (42.36 mg, 0.06 mmol) in DMF (2.00 mL) was added TEA (0.25 mL, 1.80 mmol). The reaction mixture was degassed with argon for three times and stirred for 1 h at 90° C. The resulting mixture was concentrated under reduced pressure. The residue was purified by Prep-HPLC with the following conditions: Column: Atlantis Prep T3 OBD Column, 19×250 mm 10 u; Mobile Phase A: Water (0.1% FA), Mobile Phase B: ACN; Flow rate: 20 mL/min; Gradient: 30 B to 50 B in 6 min; 210/254 nm; RT: 5.58 min. The fractions contained desired product were combined and concentrated to afford 1-((3S,5R)-1-acryloyl-5-(methoxymethyl)pyrrolidin-3-yl)-3-((1-ethyl-4-fluoro-1H-benzo[d]imidazol-5-yl)ethynyl)-5-(methylamino)-1H-pyrazole-4-carboxamide (60.0 mg, 20%) as a light pink solid. MS ESI calculated for C₂₅H₂₈FN₇O₃[M+H]⁺, 494.22, found 494.20; ¹H NMR (400 MHz, DMSO-d₆) δ 8.56 (s, 1H), 7.56-7.44 (m, 3H), 6.99-6.43 (m, 2H), 6.19-6.14 (m, 1H), 5.70-5.67 (m, 2H), 5.30-5.22 (m, 1H), 4.57-4.38 (m, 3H), 4.07-3.88 (m, 2H), 3.81-3.65 (m, 2H), 3.32 (s, 3H), 2.98 (s, 3H), 2.66-2.54 (m, 1H), 2.34-2.27 (m, 1H), 1.59-1.54 (m, 3H).

### Example 69: 1-((3S,5R)-1-Acryloyl-5-(methoxymethyl)pyrrolidin-3-yl)-5-(methylamino)-3-((2-methylquinoxalin-6-yl)ethynyl)-1H-pyrazole-4-carboxamide

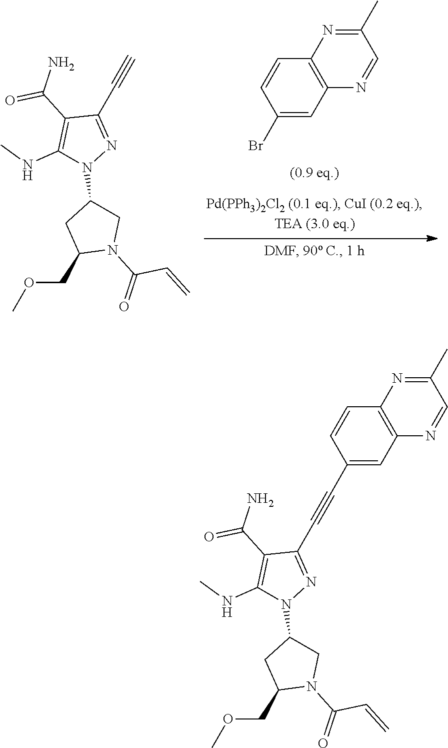

To a stirred mixture of 3-ethynyl-1-[(3S,5R)-5-(methoxymethyl)-1-(prop-2-enoyl)pyrrolidin-3-yl]-5-(methylamino)pyrazole-4-carboxamide (0.16 g, 0.48 mmol), 6-bromo-2-methylquinoxaline (96.94 mg, 0.44 mmol), CuI (18.39 mg, 0.10 mmol) and Pd(PPh₃)₂Cl₂ (33.89 mg, 0.05 mmol) in DMF (1.60 mL) was added TEA (0.20 mL, 1.99 mmol). The reaction mixture was degassed with argon for three times and stirred for 1 h at 90° C. The resulting mixture was concentrated under reduced pressure. The residue was purified by Prep-HPLC with the following conditions: Column: Atlantis Prep T3 OBD Column, 19×250 mm 10 u; Mobile Phase A: Water (0.1% FA), Mobile Phase B: ACN; Flow rate: 20 mL/min; Gradient: 25 B to 45 B in 6 min; 210/254 nm; RT: 5.56 min. The fractions contained desired product were combined and concentrated to afford 1-((3S,5R)-1-acryloyl-5-(methoxymethyl)pyrrolidin-3-yl)-5-(methylamino)-3-((2-methylquinoxalin-6-yl)ethynyl)-1H-pyrazole-4-carboxamide (39.1 mg, 17%) as a yellow solid. MS ESI calculated for C₂₅H₂₇N₇O₃ [M+H]⁺, 474.22, found 474.15; ¹H NMR (400 MHz, DMSO-d₆) δ 8.89 (s, 1H), 8.21 (s, 1H), 8.10 (d, J=8.6 Hz, 1H), 7.89 (dd, J=8.6, 1.8 Hz, 1H), 7.34 (s, 1H), 7.06-6.36 (m, 2H), 6.20-6.14 (m, 1H), 5.78-5.65 (m, 1H), 5.28-5.18 (m, 1H), 4.64-4.25 (m, 1H), 4.12-3.67 (m, 2H), 3.64-3.41 (m, 2H), 3.30 (s, 4H), 2.98-2.92 (m, 3H), 2.72 (s, 2H), 2.54 (s, 1H), 2.31-2.28 (m, 1H).

### Example 70: 3-[2-[1-(Difluoromethyl)-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide

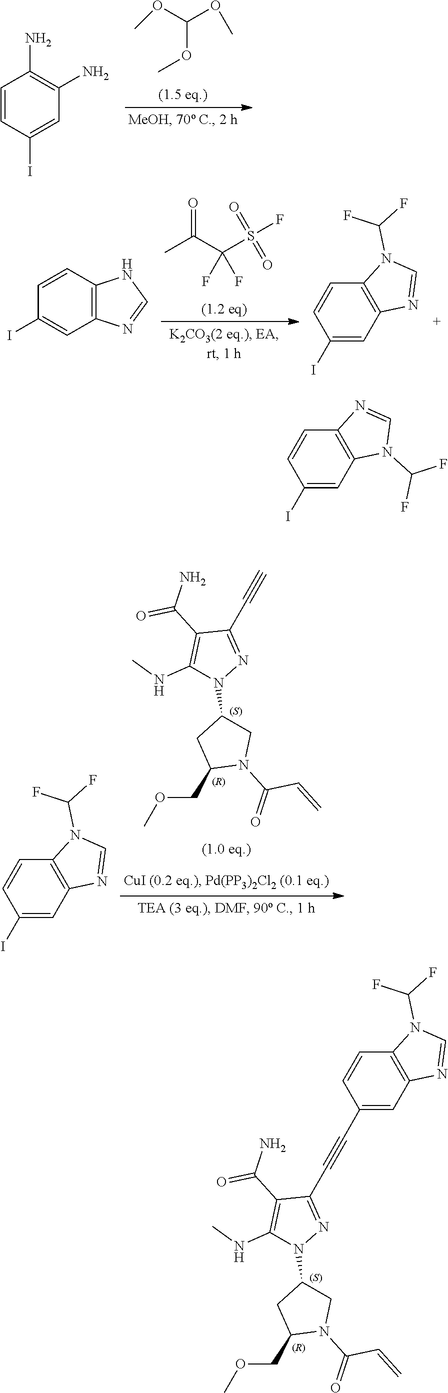

### Step 1: 5-Iodo-1H-1,3-benzodiazole

To a stirred solution of 4-iodobenzene-1,2-diamine (5.00 g, 21.36 mmol) in MeOH (50.00 mL) was added trimethyl orthoformate (6.80 g, 0.06 mmol) dropwise at room temperature under nitrogen atmosphere. The reaction mixture was stirred for 2 h at 70° C. The resulting mixture was cooled down to room temperature and concentrated under reduced pressure. The residue was purified by silica gel Column chromatography, eluted with PE/EtOAc (0˜50%). The fractions contained desired product were combined and concentrated to afford 5-iodo-1H-1,3-benzodiazole (5.5 g, 94%) as a dark yellow solid. MS ESI calculated for C₇H₅IN₂ [M+H]⁺, 244.95, found 245.00.

### Step 2: 1-(Difluoromethyl)-5-iodo-1,3-benzodiazole and 1-(difluoromethyl)-6-iodo-1,3-benzodiazole

To a stirred mixture of 5-iodo-1H-1,3-benzodiazole (5.00 g, 20.49 mmol) and K₂CO₃ (5.66 g, 0.04 mmol) in EtOAc (50.00 mL) was added 1,1-difluoro-2-oxopropane-1-sulfonyl fluoride (4.33 g, 0.03 mmol) dropwise at room temperature under nitrogen atmosphere. The reaction mixture was stirred for 1 h at room temperature. The resulting mixture was filtered. The filter cake was washed with MeOH (3×100 mL). The filtrate was concentrated under reduced pressure. The residue was purified by silica gel Column chromatography, eluted with PE/EtOAc (0˜50%). The fractions contained desired product were combined and concentrated. The crude product was purified by Prep-SFC with the following conditions Column: Chiralpak if, 30×250 mm, 5 μm; Mobile Phase A: CO₂, Mobile Phase B: MeOH (0.1% 2 M NH₃-MeOH); Flow rate: 50 mL/min; Gradient: 45% B; 220 nm; RT1: 6.2 min; RT2: 7.8 min; Injection volume: 0.5 mL; Number of runs: 60. The fractions contained desired product were combined and concentrated to afford 1-(difluoromethyl)-5-iodo-1,3-benzodiazole (1.78 g, 29%, assumed) as a yellow green solid and 1-(difluoromethyl)-6-iodo-1,3-benzodiazole (2.13 g, 35%, assumed) as a yellow green solid. MS ESI calculated for C₈H₅F₂IN₂ [M+H]⁺, 295.04, found 295.00.

### Step 3: 3-[2-[1-(Difluoromethyl)-1,3-benzodiazol-5-yl]ethynyl]-1-[(3S,5R)-5-(methoxymethyl)-1-(prop-2-enoyl) pyrrolidin-3-yl]-5-(methylamino) pyrazole-4-carboxamide

To a stirred mixture of 1-(difluoromethyl)-5-iodo-1,3-benzodiazole (0.13 g, 0.45 mmol), 3-ethynyl-1-[(3S,5R)-5-(methoxymethyl)-1-(prop-2-enoyl) pyrrolidin-3-yl]-5-(methylamino) pyrazole-4-carboxamide (0.15 g, 0.45 mmol), Pd(PPh₃)₂Cl₂ (31.80 mg, 0.05 mmol) and CuI (17.25 mg, 0.09 mmol) in DMF (2.00 mL) was added TEA (0.14 g, 1.36 mmol) at room temperature. The reaction mixture was degassed with argon for three times and stirred for 2 h at 90° C. The resulting mixture was concentrated under reduced pressure. The residue was purified by silica gel Column chromatography, eluted with CH₂Cl₂/MeOH (10/1), The fractions contained desired product were combined and concentrated. The residue was purified by reverse flash chromatography with the following conditions: Column, C18 silica gel; mobile phase, ACN in water, 5% to 70% gradient in 30 min; detector, UV 254 nm. The fractions contained desired product were combined and concentrated to afford 3-[2-[1-(difluoromethyl)-1,3-benzodiazol-5-yl]ethynyl]-1-[(3S,5R)-5-(methoxymethyl)-1-(prop-2-enoyl) pyrrolidin-3-yl]-5-(methylamino) pyrazole-4-carboxamide (80.2 mg, 33%) as an off-white solid. MS ESI calculated for C₂₄H₂₅F₂N₇O₃[M+H]⁺, 498.51, found 498.20; ¹H NMR (400 MHz, DMSO-d₆) δ 8.71 (s, 1H), 8.29-7.93 (m, 2H), 7.80 (d, J=8.4 Hz, 1H), 7.61 (dd, J=8.4, 1.5 Hz, 1H), 7.35 (s, 1H), 6.85-6.57 (m, 3H), 6.19-6.15 (m, 1H), 5.69-5.66 (m, 1H), 5.27-5.24 (m, 1H), 4.58-4.36 (m, 1H), 4.04-3.70 (m, 2H), 3.63-3.42 (m, 2H), 3.30 (d, J=5.2 Hz, 3H), 2.94 (t, J=5.1 Hz, 3H), 2.68-2.42 (m, 1H), 2.32-2.28 (m, 1H).

### Example 71: 1-((3S,5R)-1-Acryloyl-5-(methoxymethyl)pyrrolidin-3-yl)-3-((4-fluoro-1-methyl-1H-benzo[d]imidazol-5-yl)ethynyl)-5-(methylamino)-1H-pyrazole-4-carboxamide

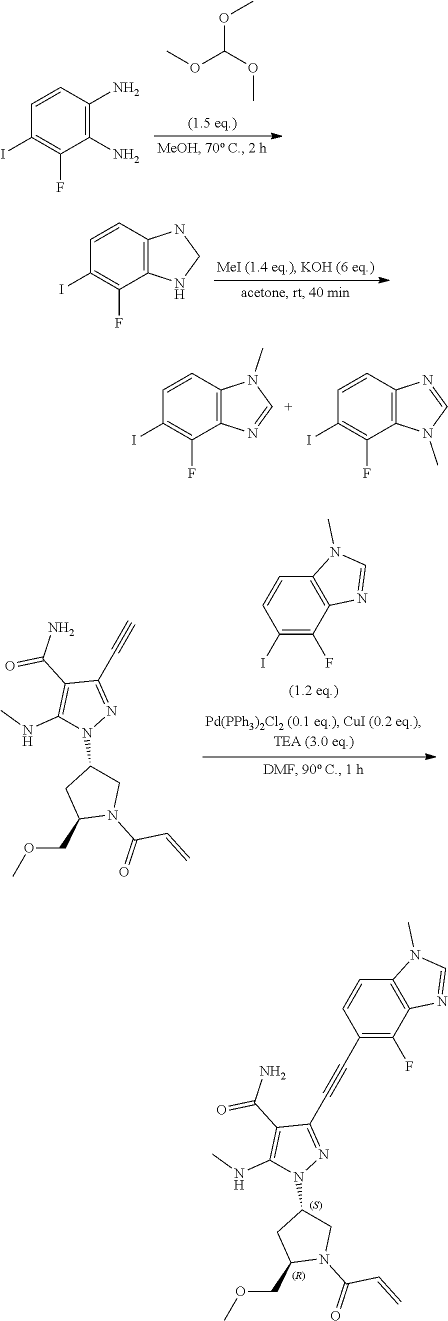

### Step 1: 4-Fluoro-5-iodo-1H-1,3-benzodiazole

To a stirred solution of 3-fluoro-4-iodobenzene-1,2-diamine (5.00 g, 19.84 mmol) in MeOH (50.00 mL) was added trimethyl orthoformate (3.16 g, 29.78 mmol) dropwise at room temperature under nitrogen atmosphere. The reaction mixture was stirred for 2 h at 70° C. under nitrogen atmosphere. The resulting mixture was concentrated under reduced pressure. The residue was purified by silica gel Column chromatography, eluted with DCM/MeOH (10/1). The fractions contained desired product were combined and concentrated to afford 4-fluoro-5-iodo-1H-1,3-benzodiazole (4.6 g, 88%) as a light yellow solid. MS ESI calculated for C₇H₄FIN₂ [M+H]⁺, 262.94, found 263.00.

### Step 2: 4-Fluoro-5-iodo-1-methyl-1,3-benzodiazole and 7-fluoro-6-iodo-1-methyl-1,3-benzodiazole

To a stirred mixture of 4-fluoro-5-iodo-1H-1,3-benzodiazole (3.50 g, 13.36 mmol) and KOH (4.50 g, 80.21 mmol) in acetone (35.00 mL) was added CH₃I (1.16 mL, 8.20 mmol) dropwise. The reaction mixture was stirred for 40 min at room temperature. The resulting mixture was diluted with water (40 mL) and extracted with EA (3×70 mL). The combined organic layers were washed with brine (50 mL), dried over anhydrous Na₂SO₄, filtered and concentrated under reduced pressure. The residue was purified by reverse flash chromatography with the following conditions: Column, C18 silica gel; mobile phase, ACN in water (10 mmol/L NH₄HCO₃), 20% to 50% gradient in 30 min; detector, UV 254 nm. The fractions contained desired product were combined and concentrated to afford 4-fluoro-5-iodo-1-methyl-1,3-benzodiazole (1.6 g, 43%) as an off-white solid. MS ESI calculated for C₈H₆FIN₂ [M+H]⁺, 276.96, found 277.00. And also afford 7-fluoro-6-iodo-1-methyl-1,3-benzodiazole (1.57 g, 42%) as an off-white solid. MS ESI calculated for C₈H₆FIN₂ [M+H]⁺, 276.96, found 277.00.

### Step 3: 1-((3S,5R)-1-Acryloyl-5-(methoxymethyl)pyrrolidin-3-yl)-3-((4-fluoro-1-methyl-1H-benzo[d]imidazol-5-yl)ethynyl)-5-(methylamino)-1H-pyrazole-4-carboxamide

To a stirred mixture of 3-ethynyl-1-[(3S,5R)-5-(methoxymethyl)-1-(prop-2-enoyl)pyrrolidin-3-yl]-5-(methylamino)pyrazole-4-carboxamide (0.2 g, 0.61 mmol), 4-fluoro-5-iodo-1-methyl-1,3-benzodiazole (0.2 g, 0.72 mmol), CuI (22.99 mg, 0.12 mmol) and Pd(PPh₃)₂Cl₂ (42.36 mg, 0.06 mmol) in DMF (2.00 mL) was added TEA (0.25 mL, 2.49 mmol). The reaction mixture was degassed with argon for three times and stirred for 1 h at 90° C. The residue was purified by Prep-HPLC with the following conditions: Column: XBridge Prep C18 OBD Column, 19×150 mm 5 μm; Mobile Phase A: Water (10 mmol/L NH₄HCO₃), Mobile Phase B: ACN; Flow rate: 20 mL/min; Gradient: 35 B to 60 B in 6 min; 210/254 nm; RT: 5.56 min. The fractions contained desired product were combined and concentrated to afford 1-((3S,5R)-1-acryloyl-5-(methoxymethyl)pyrrolidin-3-yl)-3-((4-fluoro-1-methyl-1H-benzo[d]imidazol-5-yl)ethynyl)-5-(methylamino)-1H-pyrazole-4-carboxamide (45.3 mg, 15%) as an off-white solid. MS ESI calculated for C₂₄H₂₆FN₇O₃[M+H]⁺, 480.53, found 480.20; ¹H NMR (400 MHz, DMSO-d₆) δ 8.35 (s, 1H), 7.56-7.44 (m, 3H), 6.99-6.43 (m, 3H), 6.19-6.14 (m, 1H), 5.70-5.67 (m, 1H), 5.30-5.22 (m, 1H), 4.53-4.40 (m, 1H), 4.07-3.98 (m, 1H), 3.91-3.85 (m, 4H), 3.77-3.72 (m, 1H), 3.63-3.59 (m, 1H), 3.51-3.42 (m, 3H), 2.97-2.94 (m, 3H), 2.46-2.44 (m, 1H), 2.34-2.27 (m, 1H).

### Example 72: 1-((3S,5R)-1-Acryloyl-5-(methoxymethyl)pyrrolidin-3-yl)-5-(methylamino)-3-((3-methylquinoxalin-6-yl)ethynyl)-1H-pyrazole-4-carboxamide

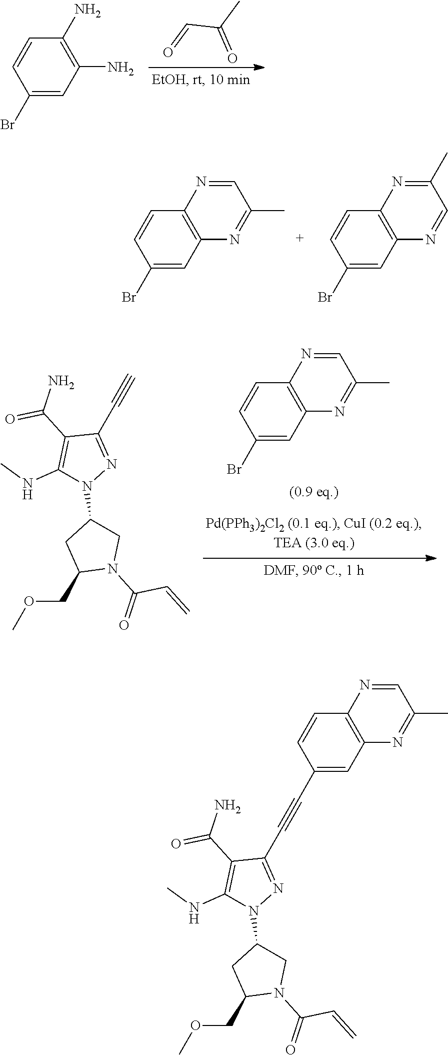

### Step 1: 7-Bromo-2-methylquinoxaline and 6-bromo-2-methylquinoxaline

To a stirred solution of 4-bromobenzene-1,2-diamine (1.00 g, 5.35 mmol) in EtOH (10.00 mL) was added 2-oxopropanal (4 mL, 40%) at room temperature under argon atmosphere. The reaction mixture was stirred for 10 min. The resulting mixture was extracted with EtOAc (3×300 mL). The combined organic layers were washed with brine (2×200 mL), dried over anhydrous Na₂SO₄ and filtered. The filtrate was concentrated under reduced pressure. The residue was purified by Prep-SFC with the following conditions Column: Chiralpak if, 30×250 mm, 5 μm; Mobile Phase A: CO₂, Mobile Phase B: IPA (0.5% 2 M NH₃-MeOH); Flow rate: 50 mL/min; Gradient: 20% B; 220 nm; RT1: 8.75 min; RT2: 10.62 min; Injection volume: 0.8 mL; Number of runs: 35. The fractions contained desired product were combined and concentrated to afford 7-bromo-2-methylquinoxaline (0.39 g, 33%) as a light brown solid. MS ESI calculated for C₉H₇BrN₂ [M+H]⁺, 222.98, found 222.95. And also afford 6-bromo-2-methylquinoxaline (0.32 g, 27%) as a light brown solid. MS ESI calculated for C₉H₇BrN₂ [M+H]⁺, 222.98, found 223.00.

### Step 2: 1-((3S,5R)-1-Acryloyl-5-(methoxymethyl)pyrrolidin-3-yl)-5-(methylamino)-3-((3-methylquinoxalin-6-yl)ethynyl)-1H-pyrazole-4-carboxamide

To a stirred mixture of 3-ethynyl-1-[(3S,5R)-5-(methoxymethyl)-1-(prop-2-enoyl)pyrrolidin-3-yl]-5-(methylamino)pyrazole-4-carboxamide (0.16 g, 0.48 mmol), 7-bromo-2-methylquinoxaline (96.94 mg, 0.44 mmol), CuI (18.39 mg, 0.10 mmol) and Pd(PPh₃)₂Cl₂ (33.89 mg, 0.05 mmol) in DMF (1.60 mL) was added TEA (0.20 mL, 1.44 mmol). The reaction mixture was degassed with argon for three times and stirred for 1 h at 90° C. The resulting mixture was concentrated under reduced pressure. The residue was purified by Prep-HPLC with the following conditions: Column: Atlantis Prep T3 OBD Column, 19×250 mm 10 u; Mobile Phase A: Water (0.1% FA), Mobile Phase B: ACN; Flow rate: 20 mL/min; Gradient: 25 B to 55 B in 6 min; 210/254 nm. The fractions contained desired product were combined and concentrated to afford 1-((3S,5R)-1-acryloyl-5-(methoxymethyl)pyrrolidin-3-yl)-5-(methylamino)-3-((3-methylquinoxalin-6-yl)ethynyl)-1H-pyrazole-4-carboxamide (52.2 mg, 23%) as a yellow solid. MS ESI calculated for C₂₅H₂₇N₇O₃ [M+H]⁺, 474.22, found 474.20; ¹H NMR (400 MHz, DMSO-d₆) δ 8.89 (s, 1H), 8.21 (s, 1H), 8.10 (d, J=8.6 Hz, 1H), 7.89-7.86 (m, 1H), 7.34 (s, 1H), 7.06-6.36 (m, 2H), 6.20-6.14 (m, 1H), 5.78-5.65 (m, 1H), 5.28-5.18 (m, 1H), 4.64-4.25 (m, 1H), 4.12-3.67 (m, 2H), 3.64-3.41 (m, 2H), 3.30 (s, 4H), 2.98-2.92 (m, 3H), 2.72 (s, 2H), 2.54 (s, 1H), 2.31-2.28 (m, 1H).

### Example 73: 3-[2-[3-(Difluoromethyl)-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide

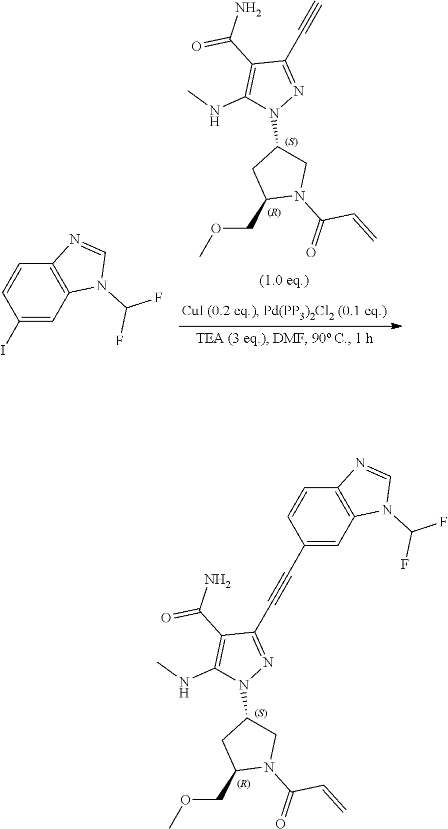

To a stirred mixture of 1-(difluoromethyl)-6-iodo-1,3-benzodiazole (0.13 g, 0.45 mmol), 3-ethynyl-1-[(3S,5R)-5-(methoxymethyl)-1-(prop-2-enoyl)pyrrolidin-3-yl]-5-(methylamino)pyrazole-4-carboxamide (0.15 g, 0.45 mmol), Pd(PPh₃)₂Cl₂ (31.77 mg, 0.05 mmol) and CuI (17.24 mg, 0.09 mmol) in DMF (2.00 mL) was added TEA (0.14 g, 1.36 mmol) at room temperature. The reaction mixture was degassed with argon for three times and stirred for 1 h at 90° C. The resulting mixture was concentrated under reduced pressure. The residue was purified by silica gel Column chromatography, eluted with CH₂Cl₂/MeOH (10/1). The fractions contained desired product were combined and concentrated. The residue was purified by reverse flash chromatography with the following conditions: Column, C18 silica gel; mobile phase, ACN in water, 5% to 70% gradient in 30 min; detector, UV 254 nm. The fractions contained desired product were combined and concentrated to afford 3-[2-[3-(difluoromethyl)-1,3-benzodiazol-5-yl]ethynyl]-1-[(3S,5R)-5-(methoxymethyl)-1-(prop-2-enoyl) pyrrolidin-3-yl]-5-(methylamino)pyrazole-4-carboxamide (88.3 mg) as an off-white solid. MS ESI calculated for C₂₄H₂₅F₂N₇O₃[M+H]⁺, 498.51, found 498.20; ¹H NMR (400 MHz, DMSO-4) S 8.73 (s, 1H), 8.30-7.94 (m, 2H), 7.84 (d, J=8.4 Hz, 1H), 7.56 (dd, J=8.4, 1.5 Hz, 1H), 7.36 (s, 1H), 6.85 (s, 1H), 6.77-6.49 (m, 2H), 6.19-6.15 (m, 1H), 5.71-5.68 (m, 1H), 5.25-5.23 (m, 1H), 4.57-4.37 (m, 1H), 4.08-3.67 (m, 2H), 3.61-3.41 (m, 2H), 3.30 (d, J=5.3 Hz, 3H), 2.94 (t, J=5.2 Hz, 3H), 2.66-2.41 (m, 1H), 2.33-2.98 (m, 1H).

### Example 74: 1-[(3S,5R)-5-(Methoxymethyl)-1-(prop-2-enoyl)pyrrolidin-3-yl]-5-(methylamino)-3-[2-(4-methylquinolin-7-yl)ethynyl]pyrazole-4-carboxamide

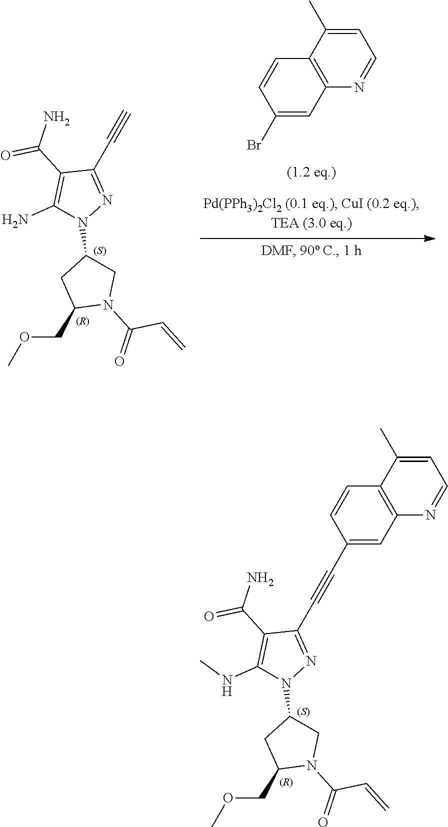

To a stirred mixture of 3-ethynyl-1-[(3S,5R)-5-(methoxymethyl)-1-(prop-2-enoyl)pyrrolidin-3-yl]-5-(methylamino)pyrazole-4-carboxamide (0.20 g, 0.60 mmol), 7-bromo-4-methylquinoline (0.16 g, 0.72 mmol), Pd(PPh₃)₂Cl₂ (42.36 mg, 0.06 mmol) and CuI (22.99 mg, 0.12 mmol) in DMF (2.00 mL) was added TEA (0.25 mL, 2.49 mmol). The reaction mixture was degassed with argon for three times and stirred for 1 h at 90° C. The resulting mixture was diluted with water (20 mL), extracted with EA (3×20 mL). The combined organic layers were washed with brine (3×20 mL), dried over anhydrous Na₂SO₄ and filtered. The filtrate was concentrated under reduced pressure. The residue was purified by silica gel Column chromatography, eluted with 6% MeOH in DCM. The fractions contained desired product were combined and concentrated. The residue was purified by Prep-HPLC with the following conditions: Column: SunFire Prep C18 OBD Column, 19×150 mm 5 μm 10 nm; Mobile Phase A: Water (0.05% TFA), Mobile Phase B: ACN; Flow rate: 20 mL/min; Gradient: 35 B to 60 B in 6 min; 210/254 nm; RT: 5.57 min. The fractions contained desired product were combined and concentrated to afford 1-[(3S,5R)-5-(methoxymethyl)-1-(prop-2-enoyl)pyrrolidin-3-yl]-5-(methylamino)-3-[2-(4-methylquinolin-7-yl)ethynyl]pyrazole-4-carboxamide (41.8 mg, 14%) as an off-white solid. MS ESI calculated for C₂₆H₂₈N₆O₃ [M+H]⁺, 473.22, found 473.30; ¹H NMR (400 MHz, DMSO-d₆): δ 8.82 (d, J=4.4 Hz, 1H), 8.26-8.07 (m, 2H), 7.74 (dd, J=8.6, 1.7 Hz, 1H), 7.44 (d, J=4.4 Hz, 2H), 6.91 (s, 1H), 6.78-6.47 (m, 2H), 6.19-6.15 (m, 1H), 5.72-5.68 (m, 1H), 5.26-5.24 (m, 1H), 4.50-4.45 (m, 1H), 4.08-3.68 (m, 2H), 3.64-3.40 (m, 2H), 3.31 (d, J=5.3 Hz, 3H), 2.95 (t, J=5.2 Hz, 3H), 2.75-2.54 (m, 4H), 2.35-2.25 (m, 1H).

### Example 75: 1-[(3S,5R)-5-(Methoxymethyl)-1-(prop-2-enoyl)pyrrolidin-3-yl]-5-(methylamino)-3-(2-[3-methylimidazo[1,2-a]pyridin-7-yl]ethynyl)pyrazole-4-carboxamide

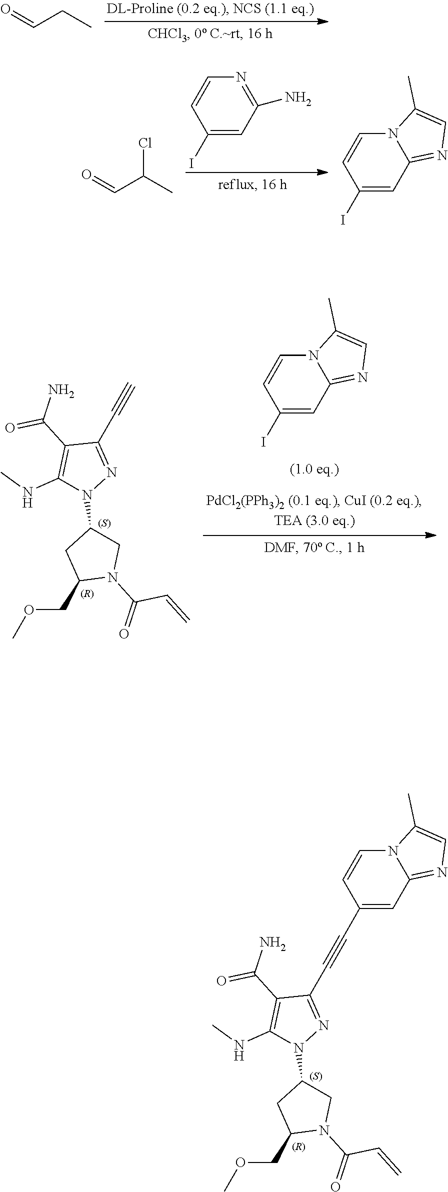

### Step 1: 2-Chloropropanal

To a stirred solution of propionaldehyde (2.50 g, 43.04 mmol) in CHCl₃ (25.00 mL) were added NCS (6.32 g, 47.35 mmol) and DL-Camphor sulfonic acid (2.00 g, 8.61 mmol) at 0° C. The reaction mixture was stirred for 16 h at room temperature. The precipitated solids were collected by filtration and washed with acetone (50 mL). The crude product 2-chloropropanal (0.85 g) was dried and used in the next step directly without further purification.

### Step 2: 7-Iodo-3-methylimidazo[1,2-a]pyridine

To a stirred solution of 2-chloropropanal (0.85 g, crude) in acetone (50.00 mL) and CHCl₃ (25.00 mL) was added 4-iodopyridin-2-amine (2.02 g, 9.19 mmol). The reaction mixture was stirred for 16 h at 70° C. under air atmosphere. The resulting mixture was concentrated under reduced pressure. The residue was purified by silica gel Column chromatography, eluted with CH₂Cl₂/MeOH (10/1). The fractions contained desired product were combined and concentrated to afford 7-iodo-3-methylimidazo[1,2-a]pyridine (80 mg, 1.43%) as a light yellow solid. MS ESI calculated for C₈H₇IN₂ [M+H]⁺, 258.97, found 259.05; ¹H NMR (400 MHz, CDCl₃) δ 8.04 (s, 1H), 7.65 (d, J=7.1 Hz, 1H), 7.38 (s, 1H), 7.10-7.07 (m, 1H), 2.47 (s, 3H).

### Step 3: 1-[(3S,5R)-5-(Methoxymethyl)-1-(prop-2-enoyl) pyrrolidin-3-yl]-5-(methylamino)-3-(2-[3-methylimidazo[1,2-a]pyridin-7-yl]ethynyl)pyrazole-4-carboxamide

To a stirred mixture of 7-iodo-3-methylimidazo[1,2-a]pyridine (0.10 g, 0.39 mmol), 3-ethynyl-1-[(3S,5R)-5-(methoxymethyl)-1-(prop-2-enoyl) pyrrolidin-3-yl]-5-(methylamino) pyrazole-4-carboxamide (0.13 g, 0.39 mmol), CuI (14.76 mg, 0.08 mmol) and Pd(PPh₃)₂Cl₂ (27.20 mg, 0.04 mmol) in DMF (2.00 mL) was added TEA (23.53 mg, 0.23 mmol). The reaction mixture was degassed with argon for three times and stirred for 1 h at 70° C. The resulting mixture was diluted with water (20 mL) and extracted with EA (3×20 mL). The combined organic layers were washed with brine (3×20 mL), dried over anhydrous Na₂SO₄ and filtered. The filtrate was concentrated under reduced pressure. The residue was purified by reverse phase chromatography with the following conditions: Column: Spherical C18, 20-40 μm, 40 g; Mobile Phase A: Water (10 mmol/L NH₄HCO₃), Mobile Phase B: ACN; Flow rate: 40 mL/min; Gradient: 5%-40% within 30 min, Detector: UV 254/220 nm; RT: 30 min. The fractions contained desired product were combined and concentrated to afford 1-[(3S,5R)-5-(methoxymethyl)-1-(prop-2-enoyl) pyrrolidin-3-yl]-5-(methylamino)-3-(2-[3-methylimidazo[1,2-a]pyridin-7-yl]ethynyl) pyrazole-4-carboxamide (67.4 mg, 37%) as an off-white solid. MS ESI calculated for C₂₄H₂₇N₇O₃ [M+H]⁺, 462.22, found 462.15; ¹H NMR (400 MHz, DMSO-d₆) δ 8.38 (s, 1H), 7.69-7.29 (m, 3H), 7.03 (d, J=7.0 Hz, 1H), 6.90-6.48 (m, 3H), 6.28-6.17 (m, 1H), 5.79-5.61 (m, 1H), 5.35-5.12 (m, 1H), 4.52-4.35 (m, 1H), 4.11-3.68 (m, 2H), 3.62-3.42 (m, 2H), 3.31 (s, 3H), 2.94 (t, J=5.2 Hz, 3H), 2.68-2.55 (m, 1H), 2.52-2.48 (m, 3H), 2.36-2.21 (m, 1H).

### Example 76: 3-[2-[3-(Difluoromethyl)-6-fluoro-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide

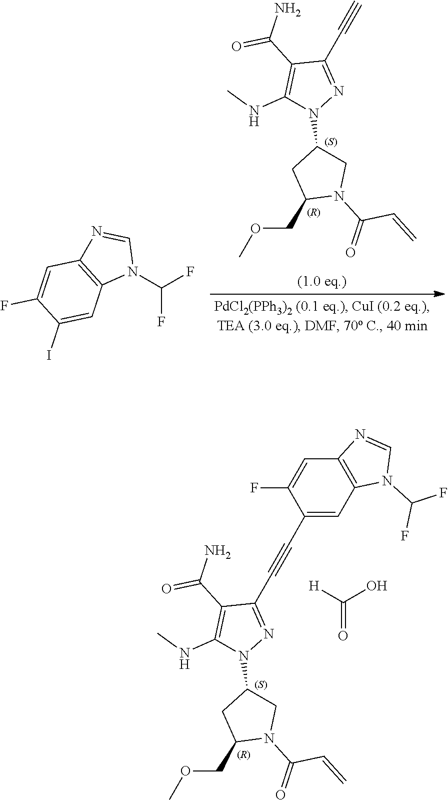

To a stirred solution of 1-(difluoromethyl)-5-fluoro-6-iodo-1,3-benzodiazole (0.10 g, 0.32 mmol), 3-ethynyl-1-[(3S,5R)-5-(methoxymethyl)-1-(prop-2-enoyl)pyrrolidin-3-yl]-5-(methylamino)pyrazole-4-carboxamide (0.11 g, 0.32 mmol), Pd(PPh₃)₂Cl₂ (22.49 mg, 0.03 mmol) and CuI (12.21 mg, 0.06 mmol) in DMF (4.00 mL) was added TEA (0.97 g, 0.96 mmol) dropwise at room temperature. The reaction mixture was degassed with argon for three times and stirred for 40 min at 70° C. The resulting mixture was cooled down and concentrated under reduced pressure. The residue was purified by silica gel Column chromatography, eluted with MeOH in DCM (0-5%) to afford the crude product. Then the crude product was purified by Prep-HPLC with the following conditions: Column: Sun-Fire Prep C18 OBD Column, 19×150 mm 5 μm 10 nm; Mobile Phase A: Water (0.1% FA), Mobile Phase B: ACN; Flow rate: 20 mL/min; Gradient: 30 B to 60 B in 6 min; 210/254 nm; RT: 5.55 min. The fractions contained desired product were combined and concentrated to afford 3-[2-[3-(difluoromethyl)-6-fluoro-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide; formic acid (0.79 g, 44%) as a white solid. MS ESI calculated for C₂₄H₂₄F₃N₇O₃[M+H]⁺, 516.19, found 516.15; ¹H NMR (400 MHz, CDCl₃) δ 8.31 (s, 1H), 7.91-7.88 (m, 1H), 7.62 (d, J=9.4 Hz, 1H), 7.43 (t, J=60.0 Hz, 1H), 7.08 (brs, 1H), 6.53-6.40 (m, 2H), 5.75-5.72 (m, 1H), 5.57-5.27 (m, 1H), 4.62-4.41 (m, 1H), 4.13-4.02 (m, 2H), 3.92-3.90 (m, 1H), 3.53-3.42 (m, 1H), 3.39 (s, 3H), 3.09-3.03 (m, 4H), 2.75-2.68 (m, 1H), 2.36-2.31 (m, 1H).

### Example 77: 3-[2-[1-(Difluoromethyl)-6-fluoro-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide

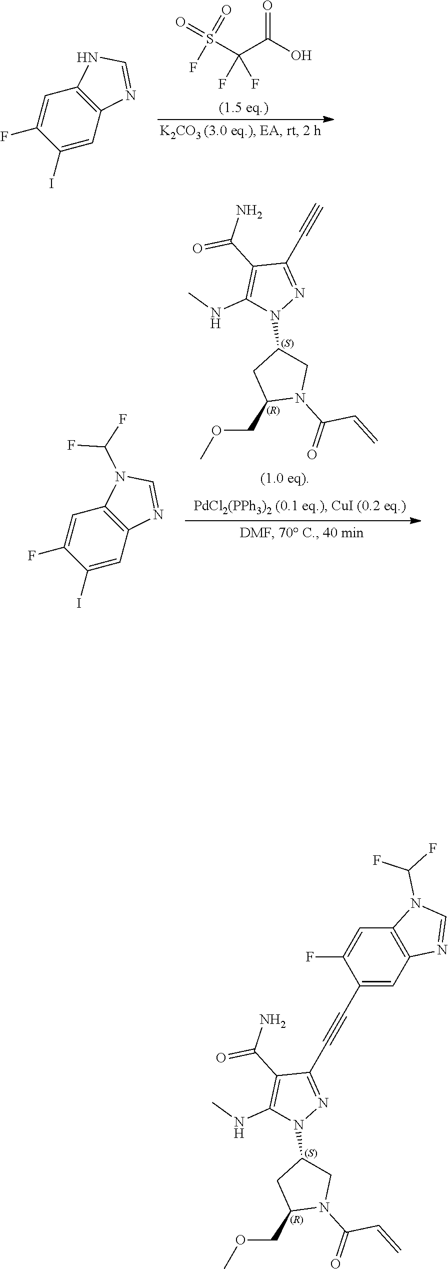

### Step 1: 1-(Difluoromethyl)-6-fluoro-5-iodo-1,3-benzodiazole

To a stirred mixture of 5-fluoro-6-iodo-3H-1,3-benzodiazole (30.00 g, 114.49 mmol) and K₂CO₃ (47.47 g, 343.48 mmol) in EA (300.00 mL) was added 2-(fluorosulfonyl) acetic acid (30.58 g, 171.74 mmol) dropwise at room temperature. The reaction mixture was stirred for 2 h at room temperature. The resulting mixture was concentrated under reduce pressure. The residue was purified by silica gel Column chromatography, eluted with EA in PE (20-80%). The fractions contained desired product were combined and concentrated. The residue was purified by Prep-SFC with the following conditions Column: Chiralpak ig, 5×25 cm, 10 μm; Mobile Phase A: CO₂, Mobile Phase B: MeOH (0.1% 2 M NH₃-MeOH); Flow rate: 200 mL/min; Gradient: 30% B; 220 nm; RT1: 4.68 min; RT2: 6.32 min; Injection volume: 6 mL; Number of runs: 33. The fractions contained desired product were combined and concentrated to afford 1-(difluoromethyl)-6-fluoro-5-iodo-1,3-benzodiazole (9.9 g, 26%) as a light yellow solid. MS ESI calculated for C₈H₄F₃IN₂ [M+H]⁺, 312.94, found 312.90; ¹H NMR (400 MHz, CDCl₃) δ 8.24 (d, J=5.5 Hz, 1H), 8.10 (s, 1H), 7.39 (d, J=7.2 Hz, 1H), 7.34 (t, J=60.0 Hz, 1H).

### Step 2: 3-[2-[1-(Difluoromethyl)-6-fluoro-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide

To a stirred mixture of 1-(difluoromethyl)-6-fluoro-5-iodo-1,3-benzodiazole (2.82 g, 9.05 mmol), 3-ethynyl-1-[(3S,5R)-5-(methoxymethyl)-1-(prop-2-enoyl)pyrrolidin-3-yl]-5-(methylamino)pyrazole-4-carboxamide (3.00 g, 9.05 mmol), Pd(PPh₃)₂Cl₂ (0.64 g, 0.90 mmol) and CuI (0.34 g, 1.81 mmol) in DMF (60.00 mL) was added TEA (2.75 g, 27.16 mmol) dropwise at room temperature. The reaction mixture was degassed with argon for three times and stirred for 40 min at 70° C. The resulting mixture was cooled down and concentrated under reduced pressure. The residue was diluted with water (200 mL) and extracted with EA (3×200 mL). The combined organic layers were washed with brine (3×200 mL), dried over anhydrous Na₂SO₄ and filtered. The filtrate was concentrated under reduced pressure. The residue was purified by silica gel Column chromatography, eluted with MeOH in DCM (0-5%). Then the crude product was purified by Prep-HPLC with the following conditions: Column: Sun-Fire Prep C18 OBD Column, 19×150 mm 5 μm 10 nm; Mobile Phase A: Water (10 mmoL/L NH₄HCO₃), Mobile Phase B: ACN; Flow rate: 100 mL/min; Gradient: 25 B to 40 B in 30 min; 220/254 nm. The fractions contained desired product were combined and concentrated to afford 3-[2-[1-(difluoromethyl)-6-fluoro-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide (1.85 g, 39%) as a white solid. MS ESI calculated for C₂₄H₂₄F₃N₇O₃[M+H]⁺, 516.19, found 516.10; ¹H NMR (400 MHz, CDCl₃) δ 8.28 (brs, 1H), 8.10 (brs, 1H), 7.47-7.40 (m, 1H), 7.34 (s, 1H), 7.05-6.69 (m, 1H), 6.31-6.33 (m, 2H), 5.73-5.69 (m, 1H), 5.63-5.25 (m, 2H), 4.51-4.42 (m, 1H), 4.23-3.84 (m, 3H), 3.63-3.41 (m, 1H), 3.37 (s, 3H), 3.07-2.98 (m, 4H), 2.76-2.68 (m, 1H), 2.42-2.30 (m, 1H).

### Example 78: 3-[2-(1-Cyclopropyl-4,6-difluoro-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

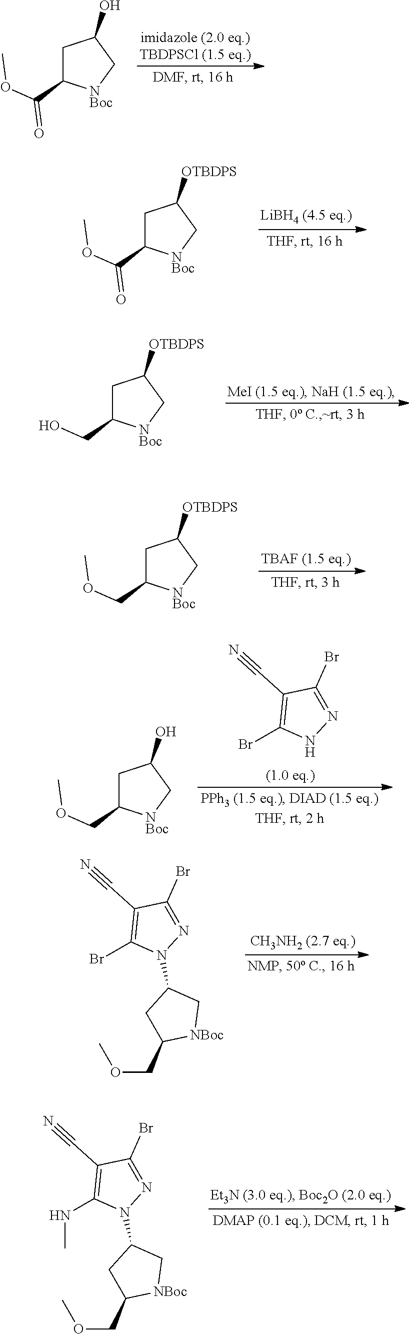

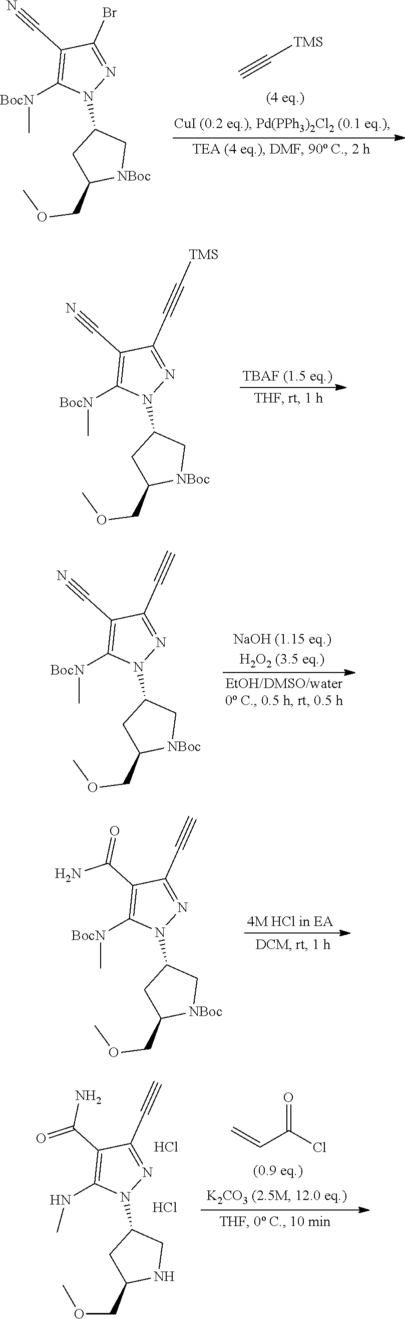

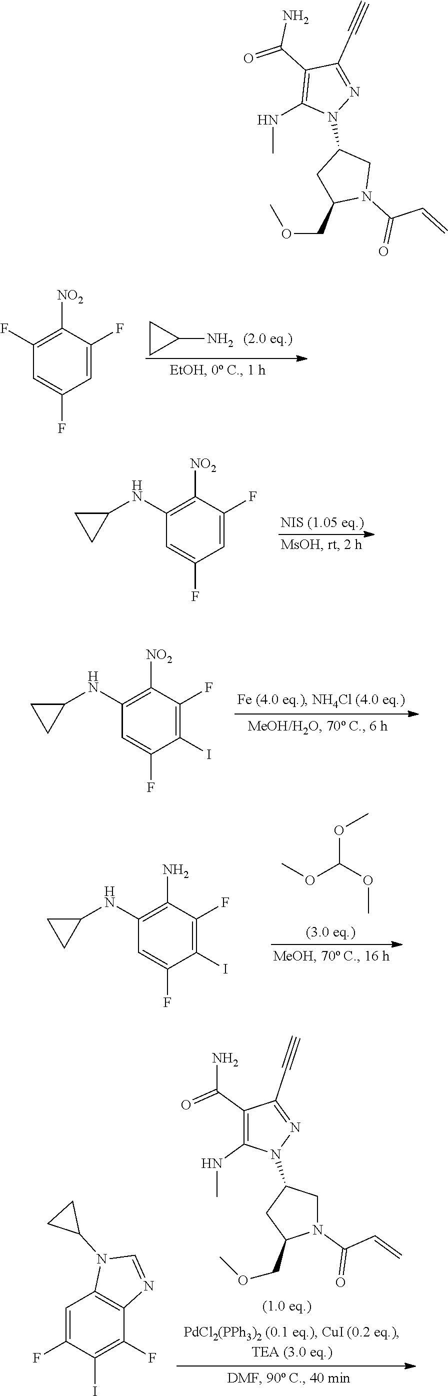

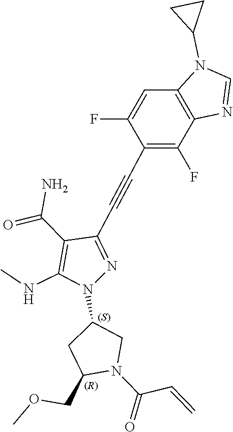

### Step 1: 1-(Tert-butyl) 2-methyl (2R,4R)-4-((tert-butyldiphenylsilyl)oxy)pyrrolidine-1,2-dicarboxylate

To a stirred solution of 1-(tert-butyl) 2-methyl (2R,4R)-4-hydroxypyrrolidine-1,2-dicarboxylate (8.00 g, 32.62 mmol) and imidazole (4.44 g, 65.23 mmol) in DMF (80.00 mL) was added tert-butyl(chloro)diphenylsilane (13.45 g, 48.93 mmol) at 0° C. over 30 min. The reaction mixture was stirred for 16 h at room temperature. The resulting mixture was diluted with water (400 mL) and extracted with EA (3×300 mL). The combined organic layers was washed with brine (5×500 mL), dried over anhydrous Na₂SO₄ and filtered. The filtrate was concentrated under reduced pressure. The residue was purified by silica gel column chromatography, eluted with PE/EA (6/1). The fractions contained desired product were combined and concentrated to afford 1-(tert-butyl) 2-methyl (2R,4R)-4-((tert-butyldiphenylsilyl)oxy)pyrrolidine-1,2-dicarboxylate (14.20 g, 90%) as a colorless oil. MS ESI calculated for C₂₇H₃₇NO₅Si [M+H]⁺, 484.24, found 484.25.

### Step 2: Tert-butyl (2R,4R)-4-[(tert-butyldiphenylsilyl)oxy]-2-(hydroxymethyl)pyrrolidine-1-carboxylate

To a stirred solution of 1-(tert-butyl) 2-methyl (2R,4R)-4-((tert-butyldiphenylsilyl)oxy)pyrrolidine-1,2-dicarboxylate (30.00 g, 62.02 mmol) in THF (300.00 mL) was added LiBH₄ (6.08 g, 0.28 mol) in portions at 0° C. under nitrogen atmosphere. The reaction mixture was stirred for 16 h at room temperature under nitrogen atmosphere. The resulting mixture was acidified to pH 5 with HCl (1M) at 0° C. and then basified to pH 8 with saturated NaHCO₃ (aq.). The resulting mixture was extracted with EA (4×500 mL). The combined organic layers was dried over anhydrous Na₂SO₄ and filtered. The filtrate was concentrated under reduced pressure. The residue was purified by silica gel column chromatography, eluted with PE/EA (5/1). The fractions contained desired product were combined and concentrated to afford tert-butyl (2R,4R)-4-[(tert-butyldiphenylsilyl)oxy]-2-(hydroxymethyl)pyrrolidine-1-carboxylate (24.00 g, 85%) as a light yellow oil. MS ESI calculated for C₂₆H₃₇NO₄Si [M+H]⁺, 456.25, found 456.30.

### Step 3: Tert-butyl (2R,4R)-4-[(tert-butyldiphenylsilyl)oxy]-2-(methoxymethyl)pyrrolidine-1-carboxylate

To a suspension of NaH (0.20 g, 8.33 mmol) in THF (18 mL) was added a solution of tert-butyl (2R,4R)-4-[(tert-butyldiphenylsilyl)oxy]-2-(hydroxymethyl)pyrrolidine-1-carboxylate (2.50 g, 5.49 mmol) in THF (64.00 mL) slowly at 0° C. under nitrogen atmosphere. After stirred at 0° C. for 1 h, to the above mixture was added CH₃I (1.17 g, 8.23 mmol) dropwise at 0° C. The reaction mixture was stirred for additional 3 h at room temperature. The resulting mixture was diluted with water (60 mL), and then extracted with EA (3×30 mL). The combined organic layers was dried over anhydrous Na₂SO₄ and filtered. The filtrate was concentrated under reduced pressure to afford tert-butyl (2R,4R)-4-[(tert-butyldiphenylsilyl)oxy]-2-(methoxymethyl)pyrrolidine-1-carboxylate (2.30 g, 89%) as a light yellow solid. MS ESI calculated for C₂₇H₃₉NO₄Si [M+H]⁺, 470.26, found 470.30.

### Step 4: Tert-butyl (2R,4R)-4-hydroxy-2-(methoxymethyl)pyrrolidine-1-carboxylate

To a stirred solution of tert-butyl (2R,4R)-4-[(tert-butyldiphenylsilyl)oxy]-2-(methoxymethyl)pyrrolidine-1-carboxylate (46.30 g, 98.57 mmol) in THF (375.00 mL) was added tetra-n-butylammonium fluoride (1 M in THF) (146.70 mL, 0.14 mol) at 0° C. The reaction mixture was stirred at room temperature for 3 h. The resulting mixture was diluted with water (1 L) and extracted with EA (3×500 mL). The combined organic layers was dried over anhydrous Na₂SO₄ and filtered. The filtrate was concentrated under reduced pressure. The residue was purified by silica gel column chromatography, eluted with PE/EA (3/1). The fractions contained desired product were combined and concentrated to afford tert-butyl (2R,4R)-4-hydroxy-2-(methoxymethyl)pyrrolidine-1-carboxylate (16.60 g, 73%) as a light yellow oil. MS ESI calculated for C₁₁H₂₁NO₄ [M+H]⁺, 232.15, found 232.20.

### Step 5: Tert-butyl (2R)-4-(3,5-dibromo-4-cyanopyrazol-1-yl)-2-(methoxymethyl)pyrrolidine-1-carboxylate

To a stirred solution of 3,5-dibromo-1H-pyrazole-4-carbonitrile (2.00 g, 7.97 mmol), tert-butyl (2R,4R)-4-hydroxy-2-(methoxymethyl)pyrrolidine-1-carboxylate (1.84 g, 7.97 mmol) and triphenylphosphine (3.14 g, 11.95 mmol) in THF (40.00 mL) was added diisopropyl azodicarboxylate (2.42 g, 11.95 mmol) dropwise at 0° C. under argon atmosphere. The reaction mixture was stirred for 2 h at room temperature. The resulting mixture was diluted with water (300 mL). The resulting mixture was extracted with EtOAc (3×200 mL). The combined organic layers was dried over anhydrous Na₂SO₄ and filtered. The filtrate was concentrated under reduced pressure. The residue was purified by silica gel column chromatography, eluted with PE/EtOAc (5/1). The fractions contained desired product were combined and concentrated to afford tert-butyl (2R)-4-(3,5-dibromo-4-cyanopyrazol-1-yl)-2-(methoxymethyl)pyrrolidine-1-carboxylate (3.50 g, 94%) as a dark yellow solid. MS ESI calculated for C₁₅H₂₀Br₂N₄O₃ [M+H−100]⁺, 361.99, 363.99, 365.99; found 362.10, 364.10, 366.10.

### Step 6: Tert-butyl (2S,4R)-4-[3-bromo-4-cyano-5-(methylamino)pyrazol-1-yl]-2-(methoxymethyl)pyrrolidine-1-carboxylate

To a stirred solution of tert-butyl (2R)-4-(3,5-dibromo-4-cyanopyrazol-1-yl)-2-(methoxymethyl)pyrrolidine-1-carboxylate (1.00 g 2.15 mmol) in NMP (10.00 mL) was added CH₃NH₂ (2.98 mL, 5.96 mmol) at room temperature under nitrogen atmosphere. The reaction mixture was stirred for 16 h at 50° C. under nitrogen atmosphere. The resulting mixture was diluted with water (30 mL) and extracted with EA (3×50 mL). The combined organic layers was washed with brine (3×20 mL), dried over anhydrous Na₂SO₄. After filtration, the filtrate was concentrated under reduced pressure. The residue was purified by silica gel column chromatography, eluted with PE/EA (3/1). The fractions contained desired product were combined and concentrated to afford tert-butyl (2S,4R)-4-[3-bromo-4-cyano-5-(methylamino)pyrazol-1-yl]-2-(methoxymethyl)pyrrolidine-1-carboxylate (0.67 g, 35%) as an off-white solid. MS ESI calculated for C₁₆H₂₄BrN₅O₃ [M+H−100]⁺, 314.11, 316.11, found 314.10, 316.10.

### Step 7: (2R,4S)-4-[3-bromo-5-[(tert-butoxycarbonyl)(methyl)amino]-4-cyanopyrazol-1-yl]-2-(methoxymethyl)pyrrolidine-1-carboxylate

To a stirred solution of tert-butyl (2R,4S)-4-[3-bromo-4-cyano-5-(methylamino)pyrazol-1-yl]-2-(methoxymethyl)pyrrolidine-1-carboxylate (20.30 g, 49.00 mmol) in DCM (300.00 mL) were added Boc₂O (20.97 mL, 98.01 mmol), DMAP (0.60 g, 4.90 mmol) and Et₃N (20.43 mL, 0.14 mol) at 0° C. under nitrogen atmosphere. The reaction mixture was stirred for 1 h at room temperature. The resulting mixture was diluted with water (3×200 mL) and extracted with DCM (3×200 mL). The combined organic layers was washed with brine (3×100 mL). The organic layer was dried over anhydrous Na₂SO₄ and filtered. The filtrate was concentrated under reduced pressure. The residue was purified by silica gel column chromatography, eluted with PE/EA (1/1). The fractions contained desired product were combined and concentrated to afford (2R,4S)-4-[3-bromo-5-[(tert-butoxycarbonyl)(methyl)amino]-4-cyanopyrazol-1-yl]-2-(methoxymethyl)pyrrolidine-1-carboxylate (24.00 g, 95%) as an off-white solid. MS ESI calculated for C₂₁H₃₂BrN₅O₅ [M+H]⁺, 514.16, 516.16, found 514.15, 516.15; ¹H NMR (400 MHz, CDCl₃) δ 4.94-4.90 (m, 1H), 4.23-4.19 (m, 1H), 3.75-3.66 (m, 3H), 3.44-3.40 (m, 1H), 3.36 (s, 3H), 3.25 (s, 3H), 2.62-2.58 (m, 1H), 2.41-2.19 (m, 1H), 1.48 (s, 18H).

### Step 8: Tert-butyl (2R,4S)-4-[5-[(tert-butoxycarbonyl)(methyl)amino]-4-cyano-3-[2-(trimethylsilyl)ethynyl]pyrazol-1-yl]-2-(methoxymethyl)pyrrolidine-1-carboxylate

To a stirred mixture of (2R,4S)-4-[3-bromo-5-[(tert-butoxycarbonyl)(methyl)amino]-4-cyanopyrazol-1-yl]-2-(methoxymethyl)pyrrolidine-1-carboxylate (24.00 g, 46.65 mmol), CuI (1.78 g, 9.33 mmol), Pd(PPh₃)₂Cl₂ (3.27 g, 4.67 mmol) and trimethylsilylacetylene (19.78 mL, 0.20 mol) in DMF (240.00 mL) was added TEA (19.45 mL, 0.19 mol). The reaction mixture was degassed with nitrogen for three times and stirred for 2 h at 90° C. The resulting mixture was concentrated under reduced pressure. The residue was diluted with water (500 mL) and extracted with EA (4×500 mL). The combined organic layers was washed with brine (2×500 mL), dried over anhydrous Na₂SO₄, filtered and the filtrate was concentrated under reduced pressure. The residue was purified by silica gel column chromatography, eluted with PE/EA (2/1). The fractions contained desired product were combined and concentrated to afford tert-butyl (2R,4S)-4-[5-[(tert-butoxycarbonyl)(methyl)amino]-4-cyano-3-[2-(trimethylsilyl)ethynyl]pyrazol-1-yl]-2-(methoxymethyl)pyrrolidine-1-carboxylate (24.00 g, 96%) as a brown solid. MS ESI calculated for C₂₆H₄₁N₅O₅Si [M+H]⁺, 532.29, found 532.40; ¹H NMR (400 MHz, CDCl₃) δ 4.93-4.89 (m, 1H), 4.23-4.17 (m, 1H), 3.68-3.52 (m, 3H), 3.42-3.37 (m, 1H), 3.35-3.33 (m, 3H), 3.25-3.20 (m, 3H), 2.63-2.58 (m, 1H), 2.33-2.13 (m, 1H), 1.46 (s, 18H), 0.27 (s, 9H).

### Step 9: Tert-butyl (2R,4S)-4-[5-[(tert-butoxycarbonyl)(methyl)amino]-4-cyano-3-ethynylpyrazol-1-yl]-2-(methoxymethyl)pyrrolidine-1-carboxylate

To a stirred solution of tert-butyl (2R,4S)-4-[5-[(tert-butoxycarbonyl)(methyl)amino]-4-cyano-3-[2-(trimethylsilyl)ethynyl]pyrazol-1-yl]-2-(methoxymethyl)pyrrolidine-1-carboxylate (24.00 g, 45.14 mmol) in THF (200.00 mL) was added TBAF (67.70 mL, 67.70 mmol, 1 M in THF) at 0° C. The reaction mixture was stirred for 1 h at room temperature. The resulting mixture was concentrated under reduced pressure. The residue was diluted with water (500 mL) and extracted with EA (3×500 mL). The combined organic layers was washed with brine (2×500 mL), dried over anhydrous Na₂SO₄, filtered and concentrated under reduced pressure. The residue was purified by silica gel column chromatography, eluted with PE/EA (2/1). The fractions contained desired product were combined and concentrated to afford tert-butyl (2R,4S)-4-[5-[(tert-butoxycarbonyl)(methyl)amino]-4-cyano-3-ethynylpyrazol-1-yl]-2-(methoxymethyl)pyrrolidine-1-carboxylate (17.40 g, 83%) as an off-white solid. MS ESI calculated for C₂₃H₃₃N₅O₅ [M+H]⁺, 460.25, found 460.40; ¹H NMR (400 MHz, CDCl₃) δ 4.93-4.89 (m, 1H), 4.22-4.18 (m, 1H), 3.84-3.46 (m, 3H), 3.42-3.37 (m, 1H), 3.37-3.31 (m, 4H), 3.24 (s, 3H), 2.62-2.59 (m, 1H), 2.28-2.24 (m, 1H), 1.46 (s, 18H).

### Step 10: Tert-butyl (2R,4S)-4-[5-[(tert-butoxycarbonyl)(methyl)amino]-4-carbamoyl-3-ethynylpyrazol-1-yl]-2-(methoxymethyl)pyrrolidine-1-carboxylate

To a stirred solution of tert-butyl (2R,4S)-4-[5-[(tert-butoxycarbonyl)(methyl)amino]-4-cyano-3-ethynylpyrazol-1-yl]-2-(methoxymethyl)pyrrolidine-1-carboxylate (17.40 g, 37.86 mmol) in DMSO (30.00 mL) and EtOH (150.00 mL) were added 0.5 M NaOH (87.09 mL, 43.54 mmol) and H₂O₂ (10.26 mL, 0.13 mol) at 0° C. The reaction mixture was stirred for 0.5 h at 0° C. Then the reaction mixture was warmed up to room temperature and stirred for another 0.5 h at room temperature. The resulting mixture was diluted with water (500 mL) and extracted with EA (3×500 mL). The combined organic layers was washed with brine (2×300 mL), dried over anhydrous Na₂SO₄, filtered and concentrated under reduced pressure. The residue was purified by silica gel column chromatography, eluted with PE/EA (1/2). The fractions contained desired product were combined and concentrated to afford tert-butyl (2R,4S)-4-[5-[(tert-butoxycarbonyl)(methyl)amino]-4-carbamoyl-3-ethynylpyrazol-1-yl]-2-(methoxymethyl)pyrrolidine-1-carboxylate (17.20 g, 95%) as an off-white solid. MS ESI calculated for C₂₃H₃₅N₅O₆ [M+H]⁺, 478.26, found 478.25; ¹H NMR (300 MHz, CDCl₃) 6.80-6.74 (m, 1H), 5.69-5.62 (m, 1H), 5.04-5.00 (m, 1H), 4.23-4.19 (m, 1H), 3.75-3.67 (m, 3H), 3.49-3.42 (m, 1H), 3.39-3.32 (m, 3H), 3.14 (s, 3H), 2.72-2.60 (m, 1H), 2.32-2.21 (m, 1H), 1.62-1.31 (m, 18H).

### Step 11: 3-Ethynyl-1-[(3S,5R)-5-(methoxymethyl)pyrrolidin-3-yl]-5-(methylamino)pyrazole-4-carboxamide dihydrochloride

To a stirred mixture of tert-butyl (2R,4S)-4-[5-[(tert-butoxycarbonyl)(methyl)amino]-4-carbamoyl-3-ethynylpyrazol-1-yl]-2-(methoxymethyl)pyrrolidine-1-carboxylate (17.20 g, 36.02 mmol) in DCM (170.00 mL) was added HCl (180.08 mL, 0.72 mol, 4 M in EA). The reaction mixture was stirred for 1 h at room temperature under argon atmosphere. The resulting mixture was concentrated and dried to afford 3-ethynyl-1-[(3S,5R)-5-(methoxymethyl)pyrrolidin-3-yl]-5-(methylamino)pyrazole-4-carboxamide dihydrochloride (12.50 g, crude) as an off-white solid which was used in the next step directly without further purification. MS ESI calculated for C₁₃H₂₁Cl₂N₅O₂ [M+H−2 HCl]⁺, 278.15, found 278.05.

### Step 12: 3-Ethynyl-1-[(3S,5R)-5-(methoxymethyl)-1-(prop-2-enoyl)pyrrolidin-3-yl]-5-(methylamino)pyrazole-4-carboxamide

To a stirred mixture of 3-ethynyl-1-[(3S,5R)-5-(methoxymethyl)pyrrolidin-3-yl]-5-(methylamino)pyrazole-4-carboxamide dihydrochloride (12.50 g, 35.69 mmol) and K₂CO₃ (172 mL, 0.43 mol, 2.5 M) in THF (250.00 mL) was added acryloyl chloride (2.89 g, 32.15 mmol) dropwise at 0° C. under argon atmosphere. The reaction mixture was stirred for 10 min at 0° C. The resulting mixture was diluted with water (500 mL) and extracted with EA (3×500 mL). The combined organic layers was washed with brine (2×300 mL), dried over anhydrous Na₂SO₄, filtered and concentrated under reduced pressure. The residue was purified by silica gel column chromatography, eluted with DCM/MeOH (10/1). The fractions contained desired product were combined and concentrated to afford 3-ethynyl-1-[(3S,5R)-5-(methoxymethyl)-1-(prop-2-enoyl)pyrrolidin-3-yl]-5-(methylamino)pyrazole-4-carboxamide (11.10 g, 84%) as an off-white solid. MS ESI calculated for C₁₆H₂₁N₅O₃ [M+H]⁺, 332.16, found 332.20; ¹H NMR (400 MHz, CDCl₃) δ 6.76 (s, 1H), 6.60-6.36 (m, 2H), 5.74-5.68 (m, 1H), 5.50-5.20 (m, 2H), 4.55-4.39 (m, 1H), 4.06-3.83 (m, 3H), 3.53-3.40 (m, 2H), 3.36-3.35 (m, 3H), 3.03-2.99 (m, 3H), 2.68-2.60 (m, 1H), 2.37-2.23 (m, 1H).

### Step 13: N-cyclopropyl-3,5-difluoro-2-nitroaniline

To a stirred solution of 1,3,5-trifluoro-2-nitrobenzene (4.50 g, 25.41 mmol) in EtOH (45.00 mL) was added aminocyclopropane (2.90 g, 50.82 mmol) dropwise at 0° C. under nitrogen atmosphere. The reaction mixture was stirred for 1 h at 0° C. The resulting mixture was diluted with water (100 mL) and extracted with EA (3×100 mL). The combined organic layers was washed with brine (2×50 mL), dried over anhydrous Na₂SO₄ and filtered. The filtrate was concentrated under reduced pressure. The residue was purified by silica gel column chromatography, eluted with PE/EA (2/1). The fractions contained desired product were combined and concentrated to afford N-cyclopropyl-3,5-difluoro-2-nitroaniline (4.11 g, 85%) as a yellow solid. ¹H NMR (400 MHz, CDCl₃) δ 7.64 (s, 1H), 6.80-6.74 (m, 1H), 6.30-6.27 (m, 1H), 2.59-2.54 (m, 1H), 1.01-0.83 (m, 2H), 0.76-0.61 (m, 2H).

### Step 14: N-cyclopropyl-3,5-difluoro-4-iodo-2-nitroaniline

To a stirred mixture of N-cyclopropyl-3,5-difluoro-2-nitroaniline (4.11 g, 19.19 mmol) in methanesulfonic acid (45.00 mL) was added NIS (4.53 g, 20.15 mmol) in portions at 0° C. The reaction mixture was stirred for 2 h at room temperature. The resulting mixture was quenched with ice/water (100 mL) at 0° C. The resulting mixture was basified to pH 8 with sat. NaOH and extracted with EA (3×100 mL). The combined organic layers was washed with brine (3×100 mL), dried over anhydrous Na₂SO₄ and filtered. The filtrate was concentrated under reduced pressure. The residue was purified by silica gel column chromatography, eluted with PE/EA (1/1). The fractions contained desired product were combined and concentrated to afford N-cyclopropyl-3,5-difluoro-4-iodo-2-nitroaniline (4.50 g, 69%) a yellow solid. ¹H NMR (400 MHz, CDCl₃) δ 7.53 (s, 1H), 6.90-6.88 (m, 1H), 2.57-2.54 (m, 1H), 1.07-0.85 (m, 2H), 0.83-0.58 (m, 2H).

### Step 15: N1-cyclopropyl-3,5-difluoro-4-iodobenzene-1,2-diamine

To a stirred mixture of N-cyclopropyl-3,5-difluoro-4-iodo-2-nitroaniline (4.40 g, 12.94 mmol) and NH₄Cl (2.77 g, 51.76 mmol) in EtOH (44.00 mL) and H₂O (8.80 mL) was added Fe (2.89 g, 51.76 mmol). The reaction mixture was stirred at 70° C. for 6 h. The resulting mixture was diluted with water (150 mL) and extracted with EA (3×100 mL). The combined organic layers was washed with brine (2×50 mL), dried over anhydrous Na₂SO₄ and filtered. The filtrate was concentrated under reduced pressure to afford N¹-cyclopropyl-3,5-difluoro-4-iodobenzene-1,2-diamine (3.30 g, crude) as a brown oil which was used in the next step directly without further purification. MS ESI calculated for C₉H₉F₂IN₂ [M+H]⁺, 310.98, found 311.00; ¹H NMR (300 MHz, CDCl₃) δ 6.68-6.61 (m, 1H), 2.50-2.41 (m, 1H), 0.89-0.73 (m, 2H), 0.78-0.51 (m, 2H).

### Step 16: 1-Cyclopropyl-4,6-difluoro-5-iodo-1,3-benzodiazole

To a stirred solution of N¹-cyclopropyl-3,5-difluoro-4-iodobenzene-1,2-diamine (3.30 g, 10.64 mmol) in MeOH (33.00 mL) was added trimethyl orthoformate (3.39 g, 31.92 mmol) at room temperature under nitrogen atmosphere. The reaction mixture was stirred for 16 h at 70° C. The resulting mixture was cooled down to room temperature and concentrated under reduced pressure. The residue was purified by silica gel column chromatography, eluted with PE/EA (1/2). The fractions contained desired product were combined and concentrated to afford 1-cyclopropyl-4,6-difluoro-5-iodo-1,3-benzodiazole (1.70 g, 50%) as a yellow solid. MS ESI calculated for C₁₀H₇F₂IN₂ [M+H]⁺, 320.96, found 321.00; ¹H NMR (300 MHz, CDCl₃) δ 7.91 (s, 1H), 7.22-7.18 (m, 1H), 3.41-3.36 (m, 1H), 1.31-1.02 (m, 4H).

### Step 17: 3-[2-(1-Cyclopropyl-4,6-difluoro-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a stirred solution of 1-cyclopropyl-4,6-difluoro-5-iodo-1,3-benzodiazole (0.97 g, 3.02 mmol), 3-ethynyl-1-[(3S,5R)-5-(methoxymethyl)-1-(prop-2-enoyl)pyrrolidin-3-yl]-5-(methylamino)pyrazole-4-carboxamide (1.00 g, 3.02 mmol), Pd(PPh₃)₂Cl₂ (0.21 g, 0.30 mmol) and CuI (0.11 g, 0.60 mmol) in DMF (15.00 mL) was added TEA (0.92 g, 9.05 mmol) dropwise at room temperature. The reaction mixture was degassed with argon for three times and stirred for 40 min at 90° C. The resulting mixture was cooled down to room temperature and concentrated under reduced pressure. The residue was purified by silica gel column chromatography, eluted with DCM/MeOH (10/1) to afford the crude product. Then the crude product was further purified by reverse phase flash with the following conditions: column: C18 silica gel; mobile phase: ACN in water (10 mmol/L NH₃HCO₃), 10% to 50% gradient in 30 min; detector: UV 254 nm. The fractions contained desired product were combined and concentrated to afford 3-[2-(1-cyclopropyl-4,6-difluoro-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (0.51 g, 33%) as a white solid. MS ESI calculated for C₂₆H₂₇F₂N₇O₃[M+H]⁺, 524.21, found 524.35; ¹H NMR (300 MHz, CDCl₃) δ 7.99 (s, 1H), 7.30-7.12 (m, 2H), 6.83 (brs, 1H), 6.67-6.32 (m, 2H), 5.84-5.73 (m, 1H), 5.64-5.12 (m, 2H), 4.71-4.38 (m, 1H), 4.25-3.84 (m, 3H), 3.60-3.33 (m, 5H), 3.20-3.08 (m, 3H), 2.86-2.70 (m, 1H), 2.37-2.31 (m, 1H), 1.40-0.89 (m, 4H).

### Example 79: 3-[2-(6-Fluoro-1-methyl-1,3-benzodiazol-5-yl)ethynyl]-5-(methylamino)-1-[(3S,5R)-1-(prop-2-enoyl)-5-[(trifluoromethoxy)methyl]pyrrolidin-3-yl]pyrazole-4-carboxamide

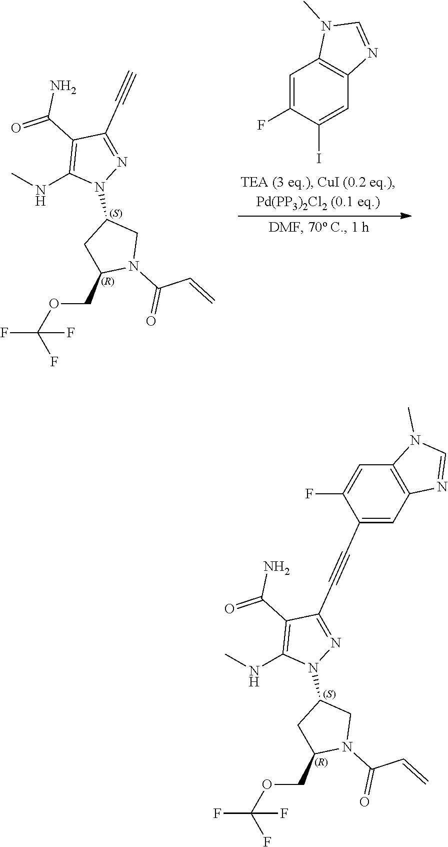

To a stirred mixture of 3-ethynyl-5-(methylamino)-1-[(3S,5R)-1-(prop-2-enoyl)-5-[(trifluoromethoxy)methyl]pyrrolidin-3-yl]pyrazole-4-carboxamide (2.50 g, 6.49 mmol), 6-fluoro-5-iodo-1-methyl-1,3-benzodiazole (1.79 g, 6.49 mmol), Pd(PPh₃)₂Cl₂ (0.46 g, 0.65 mmol) and CuI (0.25 g, 1.29 mmol) in DMF (25.00 mL) was added TEA (2.71 mL, 19.46 mmol). The reaction mixture was degassed with argon for three times and stirred for 1 h at 70° C. The resulting mixture was diluted with water (200 mL) and extracted with EA (3×200 mL). The combined organic layers were washed with brine (2×300 mL), dried over anhydrous Na₂SO₄ and filtered. The filtrate was concentrated under reduced pressure. The residue was purified by silica gel Column chromatography, eluted with 5% MeOH in DCM. The fractions contained desired product were combined and concentrated. The crude product was purified by Prep-HPLC with the following conditions: Column: XBridge Prep C18 OBD Column, Mobile Phase A: Water (10 mmol/L NH₄HCO₃), Mobile Phase B: ACN; Flow rate: 100 mL/min; Gradient: 15 B to 45 B in 30 min; 210/254 nm. The fractions contained desired product were combined and concentrated to afford 3-[2-(6-fluoro-1-methyl-1,3-benzodiazol-5-yl)ethynyl]-5-(methylamino)-1-[(3S,5R)-1-(prop-2-enoyl)-5-[(trifluoromethoxy)methyl]pyrrolidin-3-yl]pyrazole-4-carboxamide (1.54 g, 44%) as an off-white solid. MS ESI calculated for C₂₄H₂₃F₄N₇O₃[M+H]⁺, 534.18, found 534.15; ¹H NMR (400 MHz, DMSO-d₆): δ 8.35 (s, 1H), 7.96 (s, 1H), 7.71 (d, J=9.7 Hz, 1H), 7.49 (s, 1H), 6.81-6.55 (m, 3H), 6.20 (dd, J=16.7, 2.3 Hz, 1H), 5.78-5.68 (m, 1H), 5.31-5.21 (m, 1H), 4.83-4.37 (m, 2H), 4.28-4.24 (m, 1H), 4.03-3.98 (m, 2H), 3.85 (s, 3H), 3.02-2.94 (m, 3H), 2.76-2.54 (m, 1H), 2.38-2.31 (m, 1H).

### Example 80: 3-[2-(1-Ethyl-6-fluoro-1,3-benzodiazol-5-yl)ethynyl]-5-(methylamino)-1-[(3S,5R)-1-(prop-2-enoyl)-5-[(trifluoromethoxy)methyl]pyrrolidin-3-yl]pyrazole-4-carboxamide

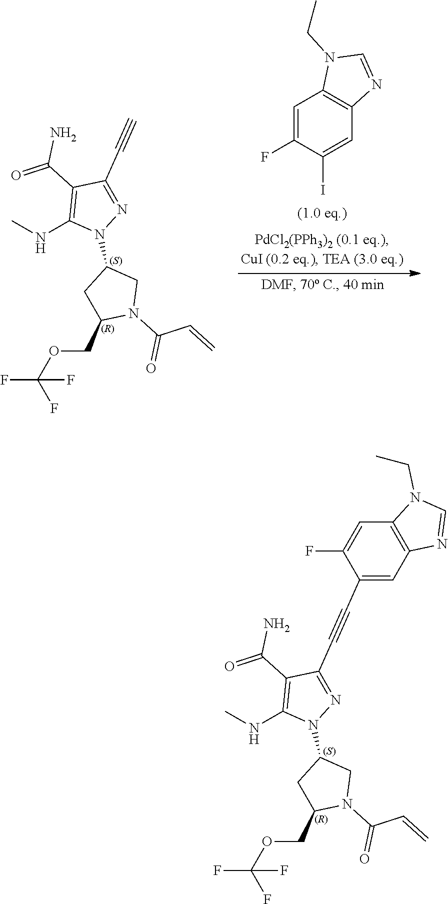

To a stirred mixture of 3-ethynyl-5-(methylamino)-1-[(3S,5R)-1-(prop-2-enoyl)-5-[(trifluoromethoxy)methyl]pyrrolidin-3-yl]pyrazole-4-carboxamide (2.40 g, 6.22 mmol) and 1-ethyl-6-fluoro-5-iodo-1,3-benzodiazole (1.81 g, 6.24 mmol) in DMF (48.00 mL) were added Pd(PPh₃)₂Cl₂ (0.44 g, 0.62 mmol), CuI (0.24 g, 1.24 mmol) and TEA (1.89 g, 18.68 mmol) dropwise over 10 min under argon atmosphere. The reaction mixture was degassed with argon for three times and stirred for 40 min at 70° C. The resulting mixture was diluted with water (300 mL) and extracted with EA (3×200 mL). The combined organic layers were washed with brine (2×300 mL), dried over anhydrous Na₂SO₄ and filtered. The filtrate was concentrated under reduced pressure. The residue was purified by silica gel Column chromatography, eluted with MeOH/DCM (0-3%). The fractions contained desired product were combined and concentrated. The residue was purified by reverse flash chromatography with the following conditions: Column, C18 silica gel; mobile phase, NH₄HCO₃ (10 mmol/L) in water, 5% to 41% gradient in 30 min; detector, UV 254 nm. The fractions contained desired product were combined and concentrated to afford 3-[2-(1-ethyl-6-fluoro-1,3-benzodiazol-5-yl)ethynyl]-5-(methylamino)-1-[(3S,5R)-1-(prop-2-enoyl)-5-[(trifluoromethoxy)methyl]pyrrolidin-3-yl]pyrazole-4-carboxamide (1.50 g, 43%) as an off-white solid. MS ESI calculated for C₂₅H₂₅F₄N₇O₃ [M+H]⁺, 548.20, found 548.10; ¹H NMR (300 MHz, DMSO-d₆) δ 8.41 (s, 1H), 7.96 (d, J=6.3 Hz, 1H), 7.76 (d, J=9.8 Hz, 1H), 7.50 (s, 1H), 6.97-6.53 (m, 3H), 6.33-6.10 (m, 1H), 5.74 (d, J=10.2 Hz, 1H), 5.25 (d, J=6.7 Hz, 1H), 4.87-4.42 (m, 2H), 4.29-4.25 (m, 3H), 4.11-3.76 (m, 2H), 3.14-2.89 (m, 3H), 2.81-2.58 (m, 1H), 2.44-2.27 (m, 1H), 1.42 (t, J=7.2 Hz, 3H).

### Example 81: 3-[2-[1-(Difluoromethyl)-6-fluoro-1,3-benzodiazol-5-yl]ethynyl]-5-(methylamino)-1-[(3S,5R)-1-(prop-2-enoyl)-5-[(trifluoromethoxy)methyl]pyrrolidin-3-yl]pyrazole-4-carboxamide

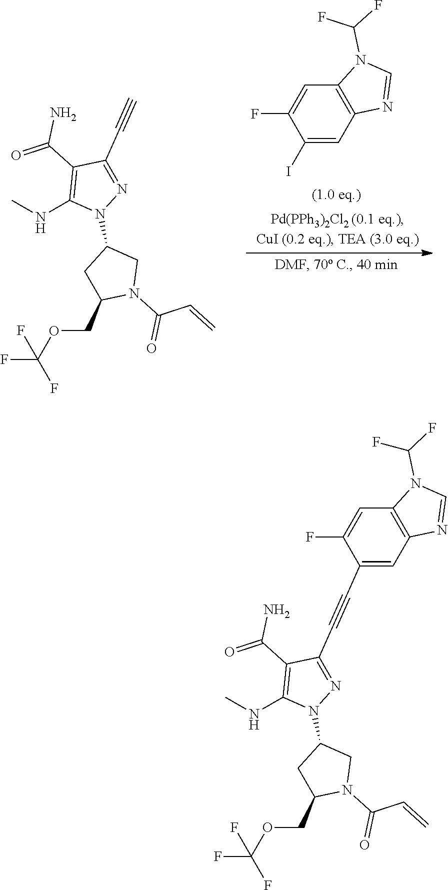 [SMILES: C#Cc1nn([C@H]2C[C@H](COC(F)(F)F)N(C(=O)C=C)C2)c(NC)c1C(N)=O ; Fc1cc2c(cc1I)ncn2C(F)F ; C=CC(=O)N1C[C@@H](n2nc(C#Cc3cc4ncn(C(F)F)c4cc3F)c(C(N)=O)c2NC)C[C@@H]1COC(F)(F)F]

To a stirred mixture of 1-[(3S,5R)-5-[(1,1-difluoroethoxy)methyl]-1-(prop-2-enoyl)pyrrolidin-3-yl]-3-ethynyl-5-(methylamino)pyrazole-4-carboxamide (0.30 g, 0.78 mmol), 1-(difluoromethyl)-6-fluoro-5-iodo-1,3-benzodiazole (0.25 g, 0.78 mmol), Pd(PPh₃)₂Cl₂ (57.32 mg, 0.08 mmol) and CuI (31.11 mg, 0.16 mmol) in DMF (2.00 mL) was added TEA (0.25 g, 2.45 mmol). The reaction mixture was degassed with argon for three times and stirred for 40 min at 70° C. The resulting mixture was diluted with water (20 mL) and extracted with EA (3×20 mL). The combined organic layers were washed with brine (3×20 mL), dried over anhydrous Na₂SO₄ and filtered. The filtrate was concentrated under reduced pressure. The residue was purified by silica gel Column chromatography, eluted with MeOH/DCM (0-5%), the fractions contained desired product were combined and concentrated. Then the crude product was purified by Prep-HPLC with the following conditions: Column: Sun-Fire Prep C18 OBD Column, 19×150 mm 5 μm 10 nm; Mobile Phase A: Water (10 mmoL/L NH₄HCO₃), Mobile Phase B: ACN; Flow rate: 20 mL/min; Gradient: 25 B to 40 B in 30 min; 220/254 nm. The fractions contained desired product were combined and concentrated to afford 3-[2-[1-(difluoromethyl)-6-fluoro-1,3-benzodiazol-5-yl]ethynyl]-5-(methylamino)-1-[(3S,5R)-1-(prop-2-enoyl)-5-[(trifluoromethoxy)methyl]pyrrolidin-3-yl]pyrazole-4-carboxamide (0.33 g, 75%) as a white solid. MS ESI calculated for C₂₄H₂₁F₆N₇O₃[M+H]⁺, 570.16, found 570.05; ¹H NMR (400 MHz, DMSO-d₆) δ 8.73 (s, 1H), 8.18-7.82 (m, 2H), 7.81-7.80 (m, 1H), 7.48 (s, 1H), 6.76-6.38 (m, 3H), 6.21-6.16 (m, 1H), 5.83-5.61 (m, 1H), 5.27-5.22 (m, 1H), 4.78-4.50 (m, 1H), 4.44-4.40 (m, 1H), 4.30-4.20 (m, 1H), 4.06-3.93 (m, 2H), 2.97-2.96 (m, 3H), 2.72-2.63 (m, 1H), 2.38-2.33 (m, 1H).

### Example 82: 3-[2-(6-Chloro-1,2-dimethyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

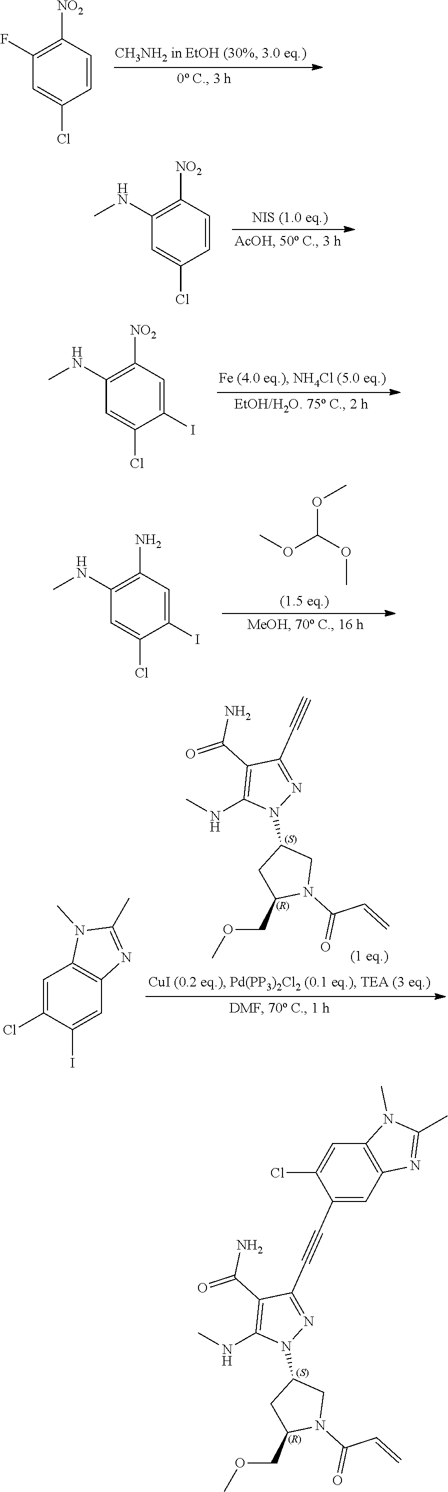

### Step 1: 5-Chloro-N-methyl-2-nitroaniline

To a stirred solution of 4-chloro-2-fluoro-1-nitrobenzene (35.00 g, 199.38 mmol) in EtOH (350.00 mL) was added methylamine (61.92 g, 598.15 mmol, 30% in ethanol) dropwise at 0° C. under N₂ atmosphere. The reaction mixture was stirred for 3 h at 0° C. The resulting mixture was quenched with water (300 mL) at 0° C. The precipitated solids were collected by filtration and washed with water (3×300 mL). The filter cake was dried to afford 5-chloro-N-methyl-2-nitroaniline (36 g, 96%) as a yellow solid which was used in the next step directly without further purification. MS ESI calculated for C₇H₇ClN₂O₂[M−H]⁻, 185.02, 187.02, found 185.00, 187.00; ¹H NMR (400 MHz, CDCl₃) δ 8.12-8.07 (m, 2H), 6.83 (d, J=2.2 Hz, 1H), 6.61 (dd, J=9.1, 2.1 Hz, 1H), 3.02 (d, J=5.1 Hz, 3H).

### Step 2: 5-Chloro-4-iodo-N-methyl-2-nitroaniline

To a stirred mixture of 5-chloro-N-methyl-2-nitroaniline (42.00 g, 225.08 mmol) in CH₃COOH (420.00 mL) was added NIS (50.64 g, 225.08 mmol) at room temperature. The reaction mixture was stirred for 3 h at 50° C. The resulting mixture was cooled down and concentrated under reduced pressure. The residue was diluted with water (400 mL) and extracted with EA (3×500 mL). The combined organic layers were washed with water (2×300 mL), dried over anhydrous Na₂SO₄ and filtered. The filtrate was concentrated under reduced pressure. The residue was purified by silica gel Column chromatography, eluted with EA in PE (0-40%). The fractions contained desired product were combined and concentrated to afford 5-chloro-4-iodo-N-methyl-2-nitroaniline (70 g, 99%) as an orange solid. MS ESI calculated for C₇H₆ClIN₂O₂[M−H]⁻, 310.92, 312.92, found 310.85, 312.85; ¹H NMR (400 MHz, CDCl₃) δ 8.74-8.52 (m, 1H), 7.99 (brs, 1H), 7.00 (d, J=1.8 Hz, 1H), 3.03 (d, J=4.7 Hz, 3H).

### Step 3: 5-Chloro-4-iodo-N1-methylbenzene-1,2-diamine

To a stirred mixture of 5-chloro-4-iodo-N-methyl-2-nitroaniline (65.00 g, 208.01 mmol) and NH₄Cl (55.63 g, 1040.03 mmol) in EtOH (650.00 mL) and H₂O (130.00 mL) was added Fe (46.46 g, 832.02 mmol). The reaction mixture was stirred for 2 h at 75° C. The resulting mixture was cooled down and filtered, the filter cake was washed with EA (2×100 mL). The filtrate was concentrated under reduced pressure. The residue was diluted with water (500 mL) and extracted with EA (3×600 mL). The combined organic layers were washed with brine (2×400 mL), dried over anhydrous Na₂SO₄ and filtered. The filtrate was concentrated and dried to afford 5-chloro-4-iodo-N¹-methylbenzene-1,2-diamine (60 g, crude) as a brown solid which was used in the next step directly without further purification. MS ESI calculated for C₇H₈ClIN₂ [M−H]⁻, 280.94, 282.94, found 281.00, 283.00; ¹H NMR (400 MHz, CDCl₃) δ 7.11 (s, 1H), 6.70 (s, 1H), 3.25 (brs, 3H), 2.84 (s, 3H).

### Step 4: 6-Chloro-5-iodo-1,2-dimethyl-1H-benzo[d]imidazole

To a stirred solution of 5-chloro-4-iodo-N¹-methylbenzene-1,2-diamine (27.80 g, 98.40 mmol) in MeOH (280.00 mL) was added 1,1,1-trimethoxyethane (18.87 mL, 147.61 mmol). The reaction mixture was stirred for 16 h at 70° C. under argon atmosphere. The resulting mixture was concentrated under reduced pressure. The residue was purified by silica gel Column chromatography, eluted with CH₂Cl₂/MeOH (10/1). The fractions contained desired product were combined and concentrated to afford 6-chloro-5-iodo-1,2-dimethyl-1H-benzo[d]imidazole (28.1 g, 93%) as a brown solid. MS ESI calculated for C₉H₈ClIN₂ [M+H]⁺, 306.94, found 307.30; ¹H NMR (400 MHz, DMSO-d₆) δ 8.06 (s, 1H), 7.87 (s, 1H), 3.71 (s, 3H), 2.52 (s, 3H).

### Step 5: 3-[2-(6-Chloro-1,2-dimethyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a stirred mixture of 3-ethynyl-1-[(3S,5R)-5-(methoxymethyl)-1-(prop-2-enoyl)pyrrolidin-3-yl]-5-(methylamino)pyrazole-4-carboxamide (2.00 g, 6.03 mmol), 6-chloro-5-iodo-1,2-dimethyl-1H-benzo[d]imidazole (1.85 g, 6.04 mmol), Pd(PPh₃)₂Cl₂ (0.42 g, 0.60 mmol) and CuI (0.23 g, 1.21 mmol) in DMF (20.00 mL) was added TEA (2.52 mL, 24.87 mmol) dropwise at room temperature. The reaction mixture was degassed with argon for three times and stirred for 1 h at 70° C. The resulting mixture was concentrated under reduced pressure. The residue was purified by silica gel Column chromatography, eluted with CH₂Cl₂/MeOH (10/1). The fractions contained desired product were combined and concentrated. The residue was purified by reverse flash chromatography with the following conditions: Column, C18 silica gel; mobile phase A: ACN, B: Water (10 mmol/L NH₄HCO₃), 10% to 40% gradient in 30 min; detector, UV 254 nm. The fractions contained desired product were combined and concentrated to afford 3-[2-(6-chloro-1,2-dimethyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (1.79 g, 58%) as a white solid. ESI calculated for C₂₅H₂₈ClN₇O₃[M+H]⁺, 510.19, found 510.15; ¹H NMR (400 MHz, DMSO-d₆) δ 7.85 (d, J=1.5 Hz, 2H), 7.51 (s, 1H), 6.86 (s, 1H), 6.78-6.54 (m, 2H), 6.17-5.96 (m, 1H), 5.69-5.45 (m, 1H), 5.27-5.03 (m, 1H), 4.52-4.41 (m, 1H), 4.04-3.95 (m, 1H), 3.89-3.65 (m, 1H), 3.75 (s, 3H), 3.61-3.56 (m, 1H), 3.52-3.42 (m, 1H), 3.33-3.30 (m, 3H), 3.00-2.93 (m, 3H), 2.64-2.58 (m, 1H), 2.54 (s, 3H), 2.29-2.12 (m, 1H).

### Example 83: 1-[(3S,5R)-5-(Methoxymethyl)-1-(prop-2-enoyl)pyrrolidin-3-yl]-5-(methylamino)-3-[2-(2-methylquinolin-7-yl)ethynyl]pyrazole-4-carboxamide

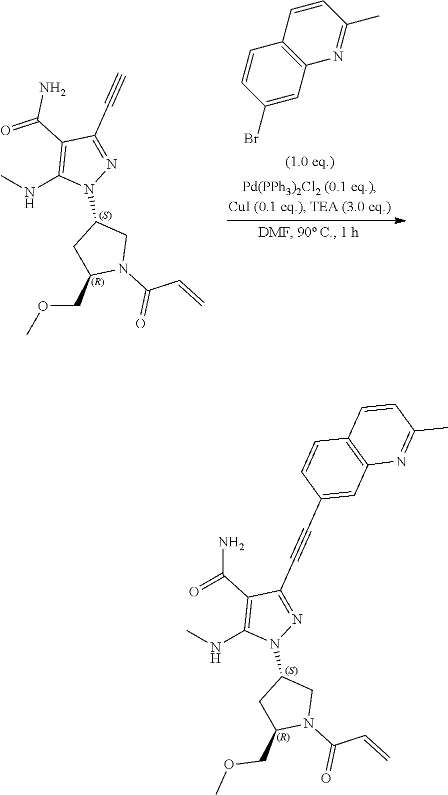

To a stirred mixture of 3-ethynyl-1-[(3S,5R)-5-(methoxymethyl)-1-(prop-2-enoyl)pyrrolidin-3-yl]-5-(methylamino)pyrazole-4-carboxamide (0.30 g, 0.9 mmol), 7-bromo-2-methylquinoline (0.20 g, 0.91 mmol), Pd(PPh₃)₂Cl₂ (63.54 mg, 0.09 mmol) and CuI (34.48 mg, 0.18 mmol) in DMF (2.00 mL) was added TEA (0.27 g, 2.72 mmol) dropwise at room temperature. The reaction mixture was degassed with argon for three times and stirred for 1 h at 90° C. The resulting mixture was concentrated under reduced pressure. The residue was purified by silica gel Column chromatography, eluted with CH₂Cl₂/MeOH (10/1). The fractions contained desired product were combined and concentrated. The residue was purified by reverse flash chromatography with the following conditions: Column, C18 silica gel; mobile phase ACN, Water (10 mmol/L, NH₄HCO₃), 10% to 40% gradient in 30 min; detector, UV 254 nm. The fractions contained desired product were combined and concentrated to afford 1-[(3S,5R)-5-(methoxymethyl)-1-(prop-2-enoyl)pyrrolidin-3-yl]-5-(methylamino)-3-[2-(2-methylquinolin-7-yl)ethynyl]pyrazole-4-carboxamide (0.15 g, 35%) as a white solid. MS ESI calculated for C₂₆H₂₈N₆O₃[M+H]⁺, 473.20, found 473.25; ¹H NMR (300 MHz, DMSO-d₆) δ 8.28 (d, J=8.5 Hz, 1H), 8.10 (s, 1H), 7.98 (d, J=8.5 Hz, 1H), 7.63 (d, J=8.4 Hz, 1H), 7.47 (d, J=8.4 Hz, 1H), 7.36 (s, 1H), 6.86 (s, 1H), 6.72-6.67 (m, 1H), 6.64-6.52 (m, 1H), 6.16 (d, J=16.6 Hz, 1H), 5.68 (d, J=10.2 Hz, 1H), 5.25 (d, J=7.5 Hz, 1H), 4.47 (d, J=39.4 Hz, 1H), 4.02-3.96 (m, 1H), 3.86 (d, J=6.7 Hz, 1H), 3.64-3.55 (m, 1H), 3.53-3.40 (m, 1H), 3.30 (d, J=5.2 Hz, 3H), 2.94-2.75 (m, 3H), 2.69-2.65 (m, 4H), 2.33-2.30 (m, 1H).

### Example 84: 3-[2-(4,6-Difluoro-1-methyl-1,3-benzodiazol-5-yl)ethynyl]-5-(methylamino)-1-[(3S,5R)-1-(prop-2-enoyl)-5-[(trifluoromethoxy)methyl]pyrrolidin-3-yl]pyrazole-4-carboxamide

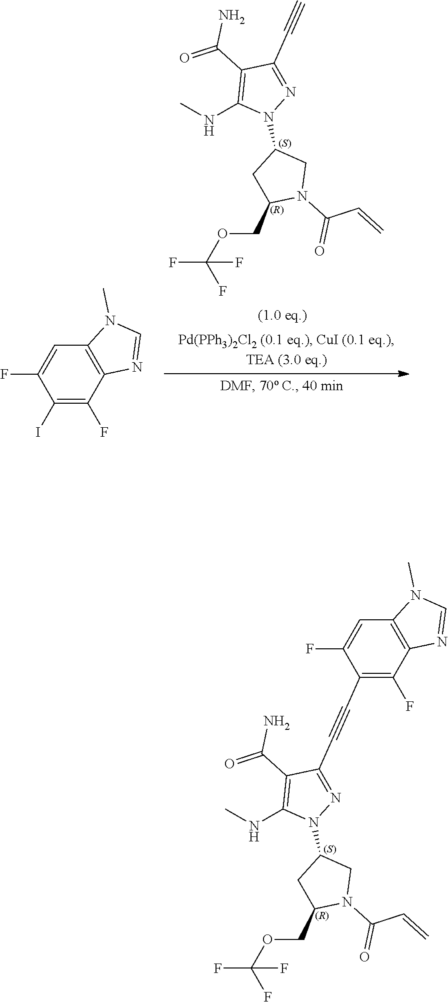

To a stirred mixture of 4,6-difluoro-5-iodo-1-methyl-1,3-benzodiazole (0.23 g, 0.78 mmol), 3-ethynyl-5-(methylamino)-1-[(3S,5R)-1-(prop-2-enoyl)-5-[(trifluoromethoxy)methyl]pyrrolidin-3-yl]pyrazole-4-carboxamide (0.30 g, 0.78 mmol), Pd(PPh₃)₂Cl₂ (54.90 mg, 0.08 mmol), CuI (29.79 mg, 0.16 mmol) in DMF (3.00 mL) was added TEA (0.24 g, 2.34 mmol) at room temperature. The reaction mixture was degassed with argon for three times and stirred for 40 min at 70° C. The resulting mixture was concentrated under reduced pressure. The residue was purified by silica gel Column chromatography, eluted with MeOH in DCM (0-4%) to afford the crude product. The crude product was purified by Prep-HPLC with the following conditions Column: XBridge Prep C18 OBD Column, 19×150 mm 5 μm; Mobile Phase A: Water (10 mmol/L NH₄HCO₃), Mobile Phase B: ACN; Flow rate: 20 mL/min; Gradient: 30 B to 60 B in 6.0 min; 210/254 nm; RT: 5.58. The fractions contained desired product were combined and concentrated to afford 3-[2-(4,6-difluoro-1-methyl-1,3-benzodiazol-5-yl)ethynyl]-5-(methylamino)-1-[(3S,5R)-1-(prop-2-enoyl)-5-[(trifluoromethoxy)methyl]pyrrolidin-3-yl]pyrazole-4-carboxamide (53.0 mg, 12%) as a white solid. MS ESI calculated for C₂₄H₂₂F₅N₇O₃ [M+H]⁺, 552.17, found 551.90; ¹H NMR (400 MHz, CDCl₃) δ 7.90 (s, 1H), 7.10-6.94 (m, 2H), 6.84 (s, 1H), 6.42 (d, J=6.1 Hz, 2H), 5.75 (t, J=6.1 Hz, 1H), 5.38-5.35 (m, 2H), 4.74-4.58 (m, 2H), 4.23-4.01 (m, 3H), 3.85 (s, 3H), 3.03 (s, 3H), 2.89-2.87 (m, 1H), 2.37-2.34 (m, 1H).

### Example 85: 3-[2-(1-Ethyl-4,6-difluoro-1,3-benzodiazol-5-yl)ethynyl]-5-(methylamino)-1-[(3S,5R)-1-(prop-2-enoyl)-5-[(trifluoromethoxy)methyl]pyrrolidin-3-yl]pyrazole-4-carboxamide

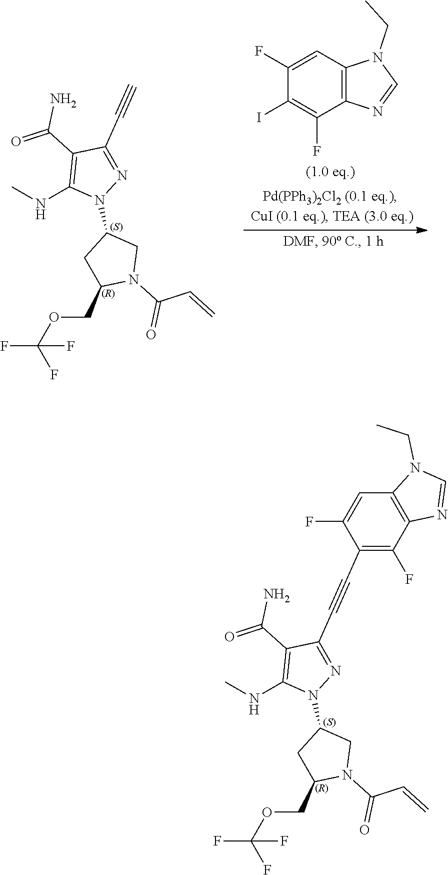

To a stirred solution of 1-ethyl-4,6-difluoro-5-iodo-1,3-benzodiazole (96.00 mg, 0.31 mmol) and 3-ethynyl-5-(methylamino)-1-[(3S,5R)-1-(prop-2-enoyl)-5-[(trifluoromethoxy)methyl]pyrrolidin-3-yl]pyrazole-4-carboxamide (0.12 g, 0.31 mmol) in DMF (3.00 mL) were added Pd(PPh₃)₂Cl₂ (21.87 mg, 0.03 mmol), CuI (11.87 mg, 0.06 mmol) and TEA (94.60 mg, 0.94 mmol) at room temperature. The reaction mixture was degassed with argon for three times and stirred for 1 h at 90° C. The resulting mixture was cooled down and concentrated under reduced pressure. The residue was purified by silica gel Column chromatography, eluted with CH₂Cl₂/MeOH (10/1). The fractions contained desired product were combined and concentrated. The residue was purified by reverse flash chromatography with the following conditions: Column, C18 silica gel; mobile phase, NH₄HCO₃ in water, 5% to 70% gradient in 30 min; detector, UV 254 nm. The fractions contained desired product were combined and concentrated to afford 3-[2-(1-ethyl-4,6-difluoro-1,3-benzodiazol-5-yl)ethynyl]-5-(methylamino)-1-[(3S,5R)-1-(prop-2-enoyl)-5-[(trifluoromethoxy)methyl]pyrrolidin-3-yl]pyrazole-4-carboxamide (64 mg, 36%) as an off-white solid. MS ESI calculated for C₂₅H₂₄F₅N₇O₃[M+H]⁺, 566.19, found 566.15; ¹H NMR (400 MHz, CDCl₃) δ 8.10 (s, 1H), 7.05 (d, J=8.3 Hz, 2H), 6.52-6.42 (m, 2H), 5.77 (t, J=6.1 Hz, 1H), 5.53-5.16 (m, 2H), 4.71-4.58 (m, 2H), 4.30-4.03 (m, 5H), 3.05 (s, 3H), 2.90-2.85 (m, 1H), 2.39-2.35 (m, 1H), 1.59 (t, J=7.3 Hz, 3H).

### Example 86: 3-[2-(6-Fluoro-1H-1,3-benzodiazol-5-yl)ethynyl]-5-(methylamino)-1-[(3S,5R)-1-(prop-2-enoyl)-5-[(trifluoromethoxy)methyl]pyrrolidin-3-yl]pyrazole-4-carboxamide

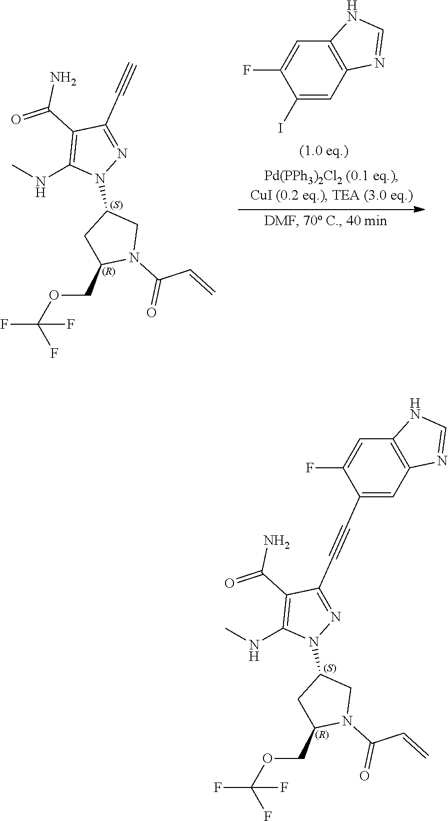

To a stirred mixture of 3-ethynyl-5-(methylamino)-1-[(3S,5R)-1-(prop-2-enoyl)-5-[(trifluoromethoxy)methyl]pyrrolidin-3-yl]pyrazole-4-carboxamide (0.15 g, 0.39 mmol), 5-fluoro-6-iodo-3H-1,3-benzodiazole (0.10 g, 0.39 mmol), Pd(PPh₃)₂Cl₂ (27.32 mg, 0.04 mmol), CuI (14.83 mg, 0.08 mmol) in DMF (4.5 mL) was added TEA (0.12 g, 1.17 mmol) at room temperature. The reaction mixture was degassed with argon for three times and stirred for 40 min at 70° C. The resulting mixture was concentrated under reduced pressure. The residue was purified by silica gel Column chromatography, eluted with MeOH in DCM (0-10%) to afford the crude product. The crude product was purified by Prep-HPLC with the following conditions Column: Atlantis HILIC OBD Column, 19×150 mm 5 μm; Mobile Phase A: Water (10 mmol/L NH₄HCO₃), Mobile Phase B: ACN; Flow rate: 20 mL/min; Gradient: 35 B to 70 B in 5.8 min; 210/254 nm; RT: 5.58 min. The fractions contained desired product were combined and concentrated to afford 3-[2-(6-fluoro-1H-1,3-benzodiazol-5-yl)ethynyl]-5-(methylamino)-1-[(3S,5R)-1-(prop-2-enoyl)-5-[(trifluoromethoxy)methyl]pyrrolidin-3-yl]pyrazole-4-carboxamide (33.3 mg, 16%) as an off-white solid. MS ESI calculated for C₂₃H₂₁F₄N₇O₃[M+H]⁺, 520.16, found 520.10; ¹H NMR (400 MHz, CDCl₃) δ 8.15 (s, 1H), 7.82 (s, 1H), 7.42 (s, 1H), 7.12 (s, 1H), 6.86 (d, J=6.1 Hz, 1H), 6.53-6.33 (m, 2H), 5.76 (dd, J=7.3, 4.9 Hz, 1H), 5.40-5.32 (m, 2H), 4.71-4.55 (m, 2H), 4.19-3.92 (m, 3H), 3.03 (d, J=5.9 Hz, 3H), 2.97-2.73 (m, 1H), 2.39-2.35 (m, 1H).

### Example 87: 3-[2-(6-Chloro-2-methyl-1H-1,3-benzodiazol-5-yl)ethynyl]-5-(methylamino)-1-[(3S,5R)-1-(prop-2-enoyl)-5-[(trifluoromethoxy)methyl]pyrrolidin-3-yl]pyrazole-4-carboxamide

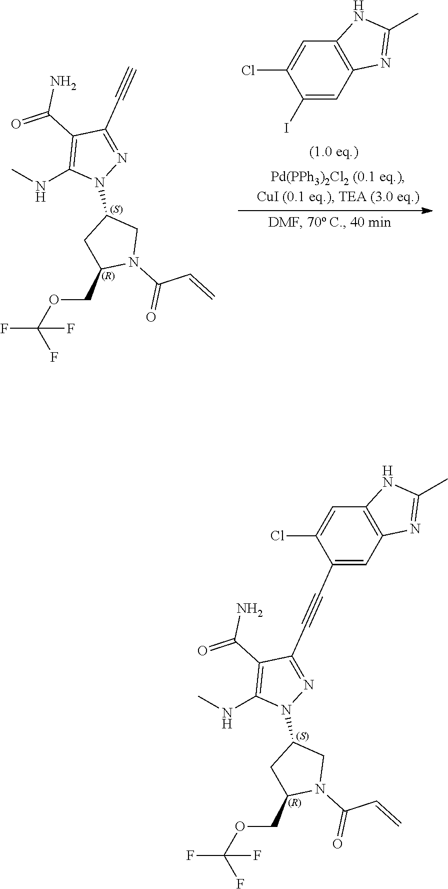

To a stirred mixture of 3-ethynyl-5-(methylamino)-1-[(3S,5R)-1-(prop-2-enoyl)-5-[(trifluoromethoxy)methyl]pyrrolidin-3-yl]pyrazole-4-carboxamide (0.15 g, 0.39 mmol), 5-chloro-6-iodo-2-methyl-3H-1,3-benzodiazole (0.11 g, 0.39 mmol), Pd(PPh₃)₂Cl₂ (27.32 mg, 0.04 mmol) and CuI (14.83 mg, 0.08 mmol) in DMF (1.50 mL, 19.38 mmol) was added TEA (0.16 mL, 1.17 mmol). The reaction mixture was degassed with argon for three times and stirred for 40 min at 70° C. The resulting mixture was concentrated under reduced pressure. The residue was purified by silica gel Column chromatography, eluted with 4% MeOH in DCM. The fractions contained desired product were combined and concentrated. The residue was purified by reverse flash chromatography with the following conditions: Column: XBridge Prep C18 OBD Column, 19×150 mm 5 μm; Mobile Phase A: Water (10 mmol/L NH₄HCO₃), Mobile Phase B: ACN; Flow rate: 20 mL/min; Gradient: 40 B to 60 B in 6 min; 210/254 nm; RT: 5.56 min. The fractions contained desired product were combined and concentrated to afford 3-[2-(6-chloro-2-methyl-1H-1,3-benzodiazol-5-yl)ethynyl]-5-(methylamino)-1-[(3S,5R)-1-(prop-2-enoyl)-5-[(trifluoromethoxy)methyl]pyrrolidin-3-yl]pyrazole-4-carboxamide (77.5 mg, 35%) as an off-white solid. MS ESI calculated for C₂₄H₂₃ClF₃N₇O₃ [M+H]⁺, 550.15, found 550.10; ¹H NMR (400 MHz, DMSO-d₆) δ 7.72 (d, J=37.9 Hz, 2H), 6.88-6.84 (m, 1H), 6.51-6.36 (m, 2H), 5.78-5.76 (m, 1H), 5.42-5.37 (m, 2H), 4.80-4.51 (m, 2H), 4.22-3.97 (m, 3H), 3.05 (d, J=5.9 Hz, 4H), 2.94-2.76 (m, 1H), 2.66 (brs, 3H), 2.40-2.35 (m, 1H).

### Example 88: 3-[2-(6-Chloro-1-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

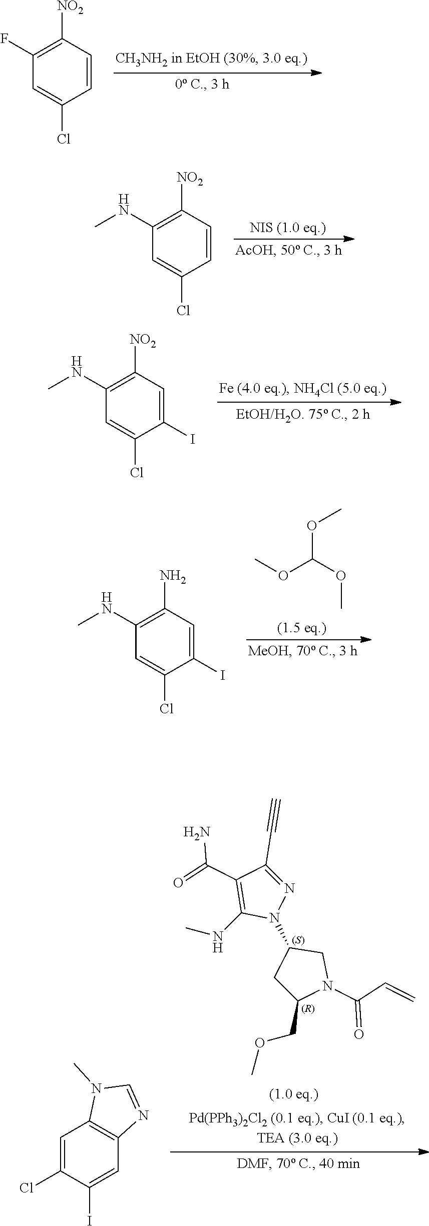

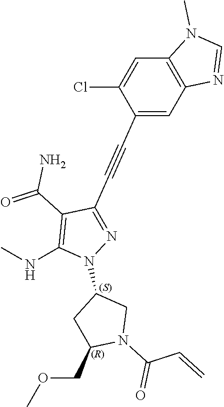

### Step 1: 5-Chloro-N-methyl-2-nitroaniline

To a stirred solution of 4-chloro-2-fluoro-1-nitrobenzene (35.00 g, 199.38 mmol) in EtOH (350.00 mL) was added methylamine in ethanol (61.92 g, 598.15 mmol, 30% in ethanol) dropwise at 0° C. under N₂ atmosphere. The reaction mixture was stirred for 3 h. The resulting mixture was quenched with water (300 mL) at 0° C. The precipitated solids were collected by filtration and washed with water (3×300 mL). The filter cake was dried to afford 5-chloro-N-methyl-2-nitroaniline (36.00 g, 96%) as a yellow solid. MS ESI calculated for C₇H₇ClN₂O₂[M−H]⁻, 185.02, found 185.00; ¹H NMR (400 MHz, CDCl₃) δ 8.12-8.07 (m, 2H), 6.83 (d, J=2.2 Hz, 1H), 6.61 (dd, J=9.1, 2.1 Hz, 1H), 3.02 (d, J=5.1 Hz, 3H).

### Step 2: 5-Chloro-4-iodo-N-methyl-2-nitroaniline

To a stirred mixture of 5-chloro-N-methyl-2-nitroaniline (42.00 g, 225.08 mmol) in CH₃COOH (420.00 mL) was added NIS (50.64 g, 225.08 mmol) at room temperature. The reaction mixture was stirred for 3 h at 50° C. The resulting mixture was cooled down and concentrated under reduced pressure. The residue was diluted with water (400 mL) and extracted with EA (3×500 mL). The combined organic layers were washed with brine (2×300 mL), dried over anhydrous Na₂SO₄ and filtered. The filtrate was concentrated under reduced pressure. The residue was purified by silica gel Column chromatography, eluted with EA in PE (0-40%). The fractions contained desired product were combined and concentrated to afford 5-chloro-4-iodo-N-methyl-2-nitroaniline (70 g, 99%) as an orange solid. MS ESI calculated for C₇H₆ClIN₂O₂[M−H]⁻, 310.92, found 310.85; ¹H NMR (400 MHz, CDCl₃) δ 8.74-8.52 (m, 1H), 7.99 (brs, 1H), 7.00 (d, J=1.8 Hz, 1H), 3.03 (d, J=4.7 Hz, 3H).

### Step 3: 5-Chloro-4-iodo-N1-methylbenzene-1,2-diamine

To a stirred mixture of 5-chloro-4-iodo-N-methyl-2-nitroaniline (65.00 g, 208.01 mmol) and NH₄Cl (55.63 g, 1040.03 mmol) in EtOH (650.00 mL) and H₂O (130.00 mL) was added Fe (46.46 g, 832.02 mmol). The reaction mixture was stirred for 2 h at 75° C. The resulting mixture was cooled down and filtered, the filter cake was washed with EA (2×100 mL). The filtrate was concentrated under reduced pressure. The residue was diluted with water (500 mL) and extracted with EA (3×600 mL). The combined organic layers were washed with brine (2×400 mL), dried over anhydrous Na₂SO₄ and filtered, The filtrate was concentrated and dried to afford 5-chloro-4-iodo-N¹-methylbenzene-1,2-diamine (60 g, 99%) as a brown solid which was used in the next step directly without further purification. MS ESI calculated for C₇H₈ClIN₂ [M−H]⁻, 280.94, found 281.00; ¹H NMR (400 MHz, CDCl₃) δ 7.11 (s, 1H), 6.70 (s, 1H), 3.25 (brs, 3H), 2.84 (s, 3H).

### Step 4: 6-Chloro-5-iodo-1-methyl-1,3-benzodiazole

To a stirred solution of 5-chloro-4-iodo-N¹-methylbenzene-1,2-diamine (60.00 g, 212.38 mmol) in MeOH (600.00 mL) was added trimethyl orthoformate (33.81 g, 318.57 mmol). The reaction mixture was stirred for 3 h at 70° C. The resulting mixture was concentrated under reduced pressure. The residue was purified by silica gel Column chromatography, eluted with EA in PE (50-100%). The fractions contained desired product were combined and concentrated to afford 6-chloro-5-iodo-1-methyl-1,3-benzodiazole (65 g, 90%) as a yellow solid. MS ESI calculated for C₈H₆ClIN₂ [M+H]⁺, 292.93, found 292.95; ¹H NMR (400 MHz, CDCl₃) δ 8.29 (s, 1H), 7.83 (s, 1H), 7.54 (s, 1H), 3.81 (s, 3H).

### Step 5: 3-[2-(6-Chloro-1-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a stirred mixture of 6-chloro-5-iodo-1-methyl-1,3-benzodiazole (0.33 g, 1.12 mmol), 3-ethynyl-1-[(3S,5R)-5-(methoxymethyl)-1-(prop-2-enoyl)pyrrolidin-3-yl]-5-(methylamino)pyrazole-4-carboxamide (0.37 g, 1.12 mmol), Pd(PPh₃)₂Cl₂ (78.37 mg, 0.11 mmol) and CuI (42.53 mg, 0.22 mmol) in DMF (5.00 mL) was added TEA (0.34 g, 3.35 mmol) dropwise at room temperature. The reaction mixture was degassed with argon for three times and stirred for 40 min at 70° C. The resulting mixture was cooled down and concentrated under reduced pressure. The residue was purified by silica gel Column chromatography, eluted with MeOH in DCM (0-5%) to afford the crude product. Then the crude product was purified by Prep-HPLC with the following conditions: Column: XBridge Prep C18 OBD Column, 19×150 mm 5 μm; Mobile Phase A: Water (10 mmol/L NH₄HCO₃), Mobile Phase B: ACN; Flow rate: 20 mL/min; Gradient: 35 B to 50 B in 6 min; 210/254 nm; RT: 5.58 min. The fractions contained desired product were combined and concentrated to afford 3-[2-(6-chloro-1-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (0.21 g, 38%) as a white solid. MS ESI calculated for C₂₄H₂₆ClN₇O₃[M+H]⁺, 496.18, found 496.05; ¹H NMR (400 MHz, CDCl₃) δ 8.10 (brs, 1H), 7.98 (brs, 1H), 7.51 (brs, 1H), 7.18 (brs, 1H), 6.81 (brs, 1H), 6.57-6.35 (m, 2H), 5.79-5.21 (m, 3H), 4.60-4.37 (m, 1H), 4.18-3.77 (m, 6H), 3.57-3.26 (m, 4H), 3.08-3.01 (m, 3H), 2.2.76-71 (m, 1H), 2.34-2.27 (m, 1H).

### Example 89: 3-[2-(6-Chloro-1-ethyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

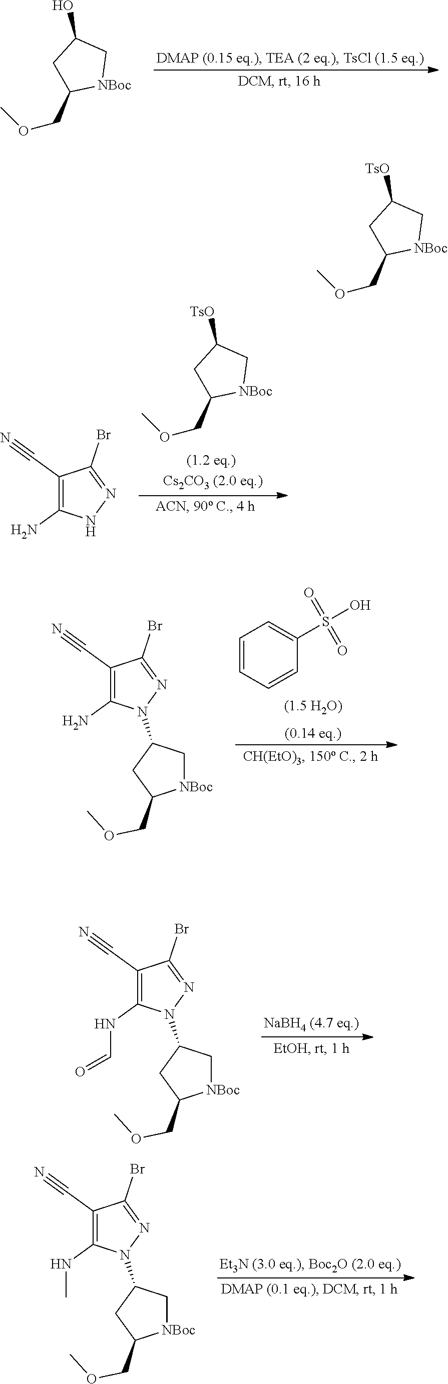

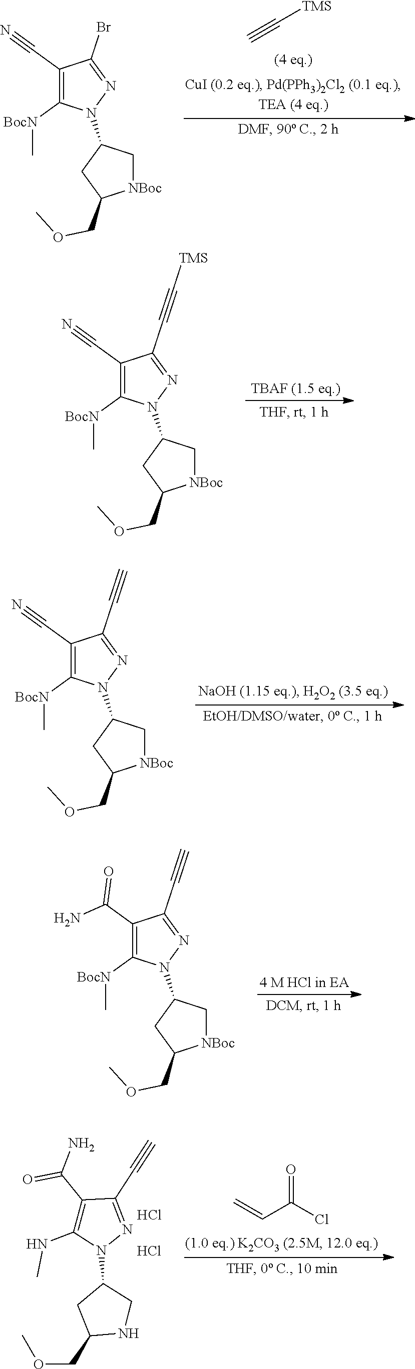

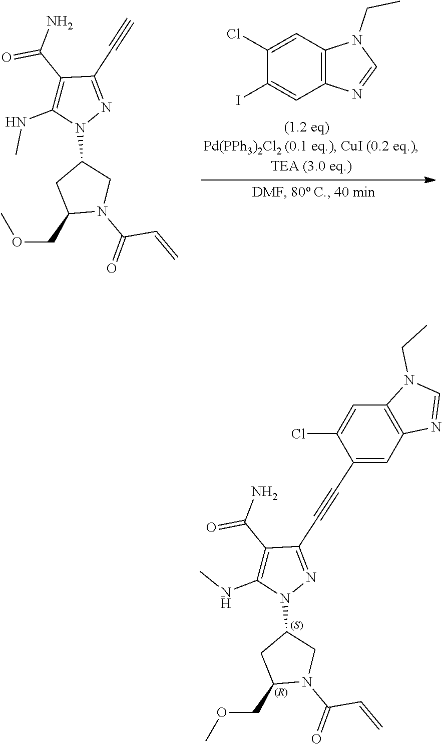

### Step 1: Tert-butyl (2R,4R)-2-(methoxymethyl)-4-(tosyloxy)pyrrolidine-1-carboxylate

To a stirred solution of tert-butyl (2R,4R)-4-hydroxy-2-(methoxymethyl)pyrrolidine-1-carboxylate (15.20 g, 65.72 mmol), TEA (18.27 mL, 131.4 mmol) and DMAP (1.21 g, 9.86 mmol) in DCM (150.00 mL) was added TsCl (18.79 g, 98.58 mmol) at 0° C. The reaction mixture was stirred for 16 h at room temperature under nitrogen atmosphere. The resulting mixture was diluted with water (80 mL) and extracted with DCM (3×150 mL). The combined organic layers were washed with brine (2×80 mL), dried over anhydrous Na₂SO₄ and filtered. The filtrate was concentrated under reduced pressure. The residue was purified by silica gel Column chromatography, eluted with 33% EA in PE. The fractions contained desired product were combined and concentrated to afford tert-butyl (2R,4R)-2-(methoxymethyl)-4-(tosyloxy)pyrrolidine-1-carboxylate (18.5 g, 73%) as a yellow oil. MS ESI calculated for C₁₈H₂₇NO₆S [M+H]⁺, 386.16, found 386.10; ¹H NMR (400 MHz, CDCl₃) δ 7.88-7.72 (m, 2H), 7.38-7.35 (m, 2H), 5.04-5.48 (m, 1H), 3.99-3.95 (m, 1H), 3.65-3.61 (m, 2H), 3.39-3.26 (m, 5H), 2.46 (s, 3H), 2.25-2.23 (m, 1H), 2.18-2.15 (m, 1H), 1.44 (s, 9H).

### Step 2: Tert-butyl (2R,4S)-4-(5-amino-3-bromo-4-cyano-1H-pyrazol-1-yl)-2-(methoxymethyl)pyrrolidine-1-carboxylate

To a mixture of 3-amino-5-bromo-2H-pyrazole-4-carbonitrile (7.20 g, 38.50 mmol) and Cs₂CO₃ (25.09 g, 77.01 mmol) in ACN (190.00 mL) was added tert-butyl (2R,4R)-2-(methoxymethyl)-4-(tosyloxy)pyrrolidine-1-carboxylate (17.81 g, 46.20 mmol). The reaction mixture was stirred for 4 h at 90° C. The resulting mixture was cooled down to room temperature and filtered. The filter cake was washed with DCM (3×70 mL). The filtrate was concentrated under reduced pressure. The residue was purified by silica gel Column chromatography, eluted with 46% EA in PE. The fractions contained desired product were concentrated. The residue was purified by reverse phase-flash chromatography with the following conditions: Column, C18 silica gel; mobile phase, ACN in water (10 mmol/L NH₄HCO₃), 5% to 46% gradient in 30 min; detector, UV 254 nm. The fractions contained desired product were combined and concentrated to afford tert-butyl (2R,4S)-4-(5-amino-3-bromo-4-cyano-1H-pyrazol-1-yl)-2-(methoxymethyl)pyrrolidine-1-carboxylate (5 g, 32%) as an off-white solid. MS ESI calculated for C₁₅H₂₂BrN₅O₃ [M+H]⁺, 400.09, 402.09, found 400.15, 402.15; ¹H NMR (400 MHz, CDCl₃) δ 4.76-4.73 (m, 1H), 4.53-4.49 (m, 2H), 4.22-4.15 (m, 1H), 3.74-3.69 (m, 3H), 3.41-3.38 (m, 1H), 3.37 (s, 3H), 2.62-2.59 (m, 1H), 2.25-2.21 (m, 1H), 1.46 (s, 9H).

### Step 3: Tert-butyl (2R,4S)-4-(3-bromo-4-cyano-5-formamido-1H-pyrazol-1-yl)-2-(methoxymethyl)pyrrolidine-1-carboxylate

To a mixture of tert-butyl (2R,4S)-4-(5-amino-3-bromo-4-cyano-1H-pyrazol-1-yl)-2-(methoxymethyl)pyrrolidine-1-carboxylate (3.50 g, 8.74 mmol) in (diethoxymethoxy)ethane (70.00 mL) was added benzenesulfonic acid (0.23 g, 1.22 mmol). The reaction mixture was stirred for 2 h at 150° C. under nitrogen atmosphere. The resulting mixture was cooled down and concentrated under reduced pressure. The residue was diluted with water (50 mL). The resulting mixture was extracted with EA (3×150 mL). The combined organic layers were washed with brine (80 mL), dried over anhydrous Na₂SO₄ and filtered. The filtrate was concentrated and dried to afford tert-butyl (2R,4S)-4-(3-bromo-4-cyano-5-formamido-1H-pyrazol-1-yl)-2-(methoxymethyl)pyrrolidine-1-carboxylate (3.75 g, crude) as a yellow oil which was used in the next step directly without further purification. MS ESI calculated for C₁₆H₂₂BrN₅O₄ [M−H]⁻, 426.09, 428.09, found 426.15, 428.15.

### Step 4: Tert-butyl (2R,4S)-4-(3-bromo-4-cyano-5-(methylamino)-1H-pyrazol-1-yl)-2-(methoxymethyl)pyrrolidine-1-carboxylate

To a stirred solution of tert-butyl (2R,4S)-4-(3-bromo-4-cyano-5-formamido-1H-pyrazol-1-yl)-2-(methoxymethyl)pyrrolidine-1-carboxylate (3.75 g, 8.76 mmol) in EtOH (200.00 mL) was added NaBH₄ (1.56 g, 41.23 mmol) at 0° C. under nitrogen atmosphere. The reaction mixture was stirred for 1 h at room temperature. The resulting mixture was quenched with brine at 0° C. and extracted with EA (3×150 mL). The combined organic layers were washed with brine (70 mL), dried over anhydrous Na₂SO₄ and filtered. The filtrate was concentrated under reduced pressure. The residue was purified by silica gel Column chromatography, eluted with 62% EA in PE. The fractions contained desired product were combined and concentrated to afford tert-butyl (2R,4S)-4-(3-bromo-4-cyano-5-(methylamino)-1H-pyrazol-1-yl)-2-(methoxymethyl)pyrrolidine-1-carboxylate (3.3 g, 91%) as an off-white solid. MS ESI calculated for C₁₆H₂₄BrN₅O₃ [M−H]⁻, 412.11, 414.11, found 412.20, 414.20; ¹H NMR (400 MHz, CDCl₃): 4.64-4.61 (m, 1H), 4.43-4.20 (m, 2H), 3.71-3.63 (m, 3H), 3.40-3.37 (m, 1H), 3.36 (s, 3H), 3.21 (s, 3H), 2.62-2.58 (m, 1H), 2.22-2.17 (m, 1H), 1.46 (s, 9H).

### Step 5: Tert-butyl (2R,4S)-4-[3-bromo-5-[(tert-butoxycarbonyl)(methyl)amino]-4-cyanopyrazol-1-yl]-2-(methoxymethyl)pyrrolidine-1-carboxylate

To a stirred solution of tert-butyl (2R,4S)-4-[3-bromo-4-cyano-5-(methylamino)pyrazol-1-yl]-2-(methoxymethyl)pyrrolidine-1-carboxylate (20.30 g, 49.00 mmol) in DCM (300.00 mL) were added Boc₂O (20.97 mL, 98.01 mmol), DMAP (0.60 g, 4.90 mmol) and Et₃N (20.43 mL, 146.98 mmol) at 0° C. under nitrogen atmosphere. The reaction mixture was stirred for 1 h at rt. The resulting mixture was washed with water (3×200 mL) and brine (200 mL). The organic layer was dried over anhydrous Na₂SO₄ and filtered. The filtrate was concentrated under reduced pressure. The residue was purified by silica gel Column chromatography, eluted with 39% EA in PE. The fractions contained desired product were combined and concentrated to afford tert-butyl (2R,4S)-4-[3-bromo-5-[(tert-butoxycarbonyl)(methyl)amino]-4-cyanopyrazol-1-yl]-2-(methoxymethyl)pyrrolidine-1-carboxylate (24.0 g, 95%) as an off-white solid. MS ESI calculated for C₂₁H₃₂BrN₅O₅ [M+H]⁺, 516.16, found 516.15; ¹H NMR (400 MHz, CDCl₃) δ 4.94-4.90 (m, 1H), 4.23-4.19 (m, 1H), 3.75-3.66 (m, 3H), 3.44-3.40 (m, 1H), 3.36 (s, 3H), 3.25 (s, 3H), 2.62-2.58 (m, 1H), 2.41-2.19 (m, 1H), 1.48 (s, 18H).

### Step 6: Tert-butyl (2R,4S)-4-[5-[(tert-butoxycarbonyl)(methyl)amino]-4-cyano-3-[2-(trimethylsilyl)ethynyl]pyrazol-1-yl]-2-(methoxymethyl)pyrrolidine-1-carboxylate

To a stirred mixture of tert-butyl (2R,4S)-4-[3-bromo-5-[(tert-butoxycarbonyl)(methyl)amino]-4-cyanopyrazol-1-yl]-2-(methoxymethyl)pyrrolidine-1-carboxylate (24.00 g, 46.65 mmol), CuI (1.78 g, 9.33 mmol), Pd(PPh₃)₂Cl₂ (3.27 g 4.67 mmol) and trimethylsilylacetylene (19.78 mL, 201.39 mmol) in DMF (240 mL) was added TEA (19.45 mL, 192.26 mmol) at room temperature. The reaction mixture was degassed with nitrogen for three times and stirred for 2 h at 90° C. The resulting mixture was concentrated under reduced pressure. The residue was diluted with water (500 mL) and extracted with EA (4×500 mL). The combined organic layers were washed with brine (2×500 mL), dried over anhydrous Na₂SO₄, filtered and concentrated under reduced pressure. The residue was purified by silica gel Column chromatography, eluted with 25% EA in PE. The fractions contained desired product were combined and concentrated to afford tert-butyl (2R,4S)-4-[5-[(tert-butoxycarbonyl)(methyl)amino]-4-cyano-3-[2-(trimethylsilyl)ethynyl]pyrazol-1-yl]-2-(methoxymethyl)pyrrolidine-1-carboxylate (24 g, 96%) as a brown solid. MS ESI calculated for C₂₆H₄₁N₅O₅Si [M+H]⁺, 532.29, found 532.40; ¹H NMR (400 MHz, CDCl₃) δ 4.93-4.89 (m, 1H), 4.23-4.17 (m, 1H), 3.68-3.52 (m, 3H), 3.42-3.37 (m, 1H), 3.35-3.33 (m, 3H), 3.25-3.20 (m, 3H), 2.63-2.58 (m, 1H), 2.33-2.13 (m, 1H), 1.46 (s, 18H), 0.27 (s, 9H).

### Step 7: Tert-butyl (2R,4S)-4-[5-[(tert-butoxycarbonyl)(methyl)amino]-4-cyano-3-ethynylpyrazol-1-yl]-2-(methoxymethyl)pyrrolidine-1-carboxylate

To a stirred solution of tert-butyl (2R,4S)-4-[5-[(tert-butoxycarbonyl)(methyl)amino]-4-cyano-3-[2-(trimethylsilyl)ethynyl]pyrazol-1-yl]-2-(methoxymethyl)pyrrolidine-1-carboxylate (24.00 g, 45.14 mmol) in THF (200.00 mL) was added TBAF (67.70 mL, 67.70 mmol, 1 M in THF) at 0° C. The reaction mixture was stirred for 1 h at room temperature. The resulting mixture was concentrated under reduced pressure. The residue was diluted with water (500 mL) and extracted with EA (3×500 mL). The combined organic layers were washed with brine (2×500 mL), dried over anhydrous Na₂SO₄, filtered and concentrated under reduced pressure. The residue was purified by silica gel Column chromatography, eluted with 30% EA in PE. The fractions contained desired product were combined and concentrated to afford tert-butyl (2R,4S)-4-[5-[(tert-butoxycarbonyl)(methyl)amino]-4-cyano-3-ethynylpyrazol-1-yl]-2-(methoxymethyl)pyrrolidine-1-carboxylate (17.4 g, 83%) as an off-white solid. MS ESI calculated for C₂₃H₃₃N₅O₅ [M+H]⁺, 460.25, found 460.40; ¹H NMR (400 MHz, CDCl₃) δ 4.93-4.89 (m, 1H), 4.22-4.18 (m, 1H), 3.84-3.46 (m, 3H), 3.42-3.37 (m, 1H), 3.37-3.31 (m, 4H), 3.24 (s, 3H), 2.62-2.59 (m, 1H), 2.28-2.24 (m, 1H), 1.46 (s, 18H).

### Step 8: Tert-butyl (2R,4S)-4-[5-[(tert-butoxycarbonyl)(methyl)amino]-4-carbamoyl-3-ethynylpyrazol-1-yl]-2-(methoxymethyl)pyrrolidine-1-carboxylate

To a stirred solution of tert-butyl (2R,4S)-4-[5-[(tert-butoxycarbonyl)(methyl)amino]-4-cyano-3-ethynylpyrazol-1-yl]-2-(methoxymethyl)pyrrolidine-1-carboxylate (17.4 g, 37.86 mmol) in DMSO (30.00 mL) and EtOH (150.00 mL) were added 0.5 M NaOH (87.09 mL, 43.54 mmol) and H₂O₂ (10.26 mL, 132.14 mmol) at 0° C. The reaction mixture was stirred for 0.5 h at 0° C. and 0.5 h at room temperature. The resulting mixture was diluted with water (500 mL) and extracted with EA (3×500 mL). The combined organic layers were washed with brine (2×300 mL), dried over anhydrous Na₂SO₄, filtered and concentrated under reduced pressure. The residue was purified by silica gel Column chromatography, eluted with 65% EA in PE. The fractions contained desired product were combined and concentrated to afford tert-butyl (2R,4S)-4-[5-[(tert-butoxycarbonyl)(methyl)amino]-4-carbamoyl-3-ethynylpyrazol-1-yl]-2-(methoxymethyl)pyrrolidine-1-carboxylate (17.2 g, 95%) as an off-white solid. MS ESI calculated for C₂₃H₃₅N₅O₆ [M+H]⁺, 478.26, found 478.25; ¹H NMR (300 MHz, CDCl₃) 6.80 (brs, 1H), 5.67 (brs, 1H), 5.04-5.00 (m, 1H), 4.23-4.19 (m, 1H), 3.75-3.67 (m, 3H), 3.49-3.42 (m, 1H), 3.39-3.32 (m, 3H), 3.14 (s, 3H), 2.72-2.60 (m, 1H), 2.32-2.21 (m, 1H), 1.62-1.31 (m, 18H).

### Step 9: 3-Ethynyl-1-[(3S,5R)-5-(methoxymethyl)pyrrolidin-3-yl]-5-(methylamino)pyrazole-4-carboxamide dihydrochloride

To a stirred mixture of tert-butyl (2R,4S)-4-[5-[(tert-butoxycarbonyl)(methyl)amino]-4-carbamoyl-3-ethynylpyrazol-1-yl]-2-(methoxymethyl)pyrrolidine-1-carboxylate (17.2 g, 36.02 mmol) in DCM (170.00 mL) was added HCl (180.08 mL, 720.32 mmol, 4 M in EA). The reaction mixture was stirred for 1 h at room temperature under argon atmosphere. The resulting mixture was concentrated and dried to afford 3-ethynyl-1-[(3S,5R)-5-(methoxymethyl)pyrrolidin-3-yl]-5-(methylamino)pyrazole-4-carboxamide dihydrochloride (12.5 g, 99%) as an off-white solid which was used in the next step directly without further purification. MS ESI calculated for C₁₃H₂₁Cl₂N₅O₂ [M+H−2 HCl]⁺, 278.15, found 278.05.

### Step 10: 3-Ethynyl-1-[(3S,5R)-5-(methoxymethyl)-1-(prop-2-enoyl)pyrrolidin-3-yl]-5-(methylamino)pyrazole-4-carboxamide

To a stirred mixture of 3-ethynyl-1-[(3S,5R)-5-(methoxymethyl)pyrrolidin-3-yl]-5-(methylamino)pyrazole-4-carboxamide dihydrochloride (12.50 g, 35.69 mmol) and K₂CO₃ (172 mL, 430 mmol, 2.5 M) in THF (250.00 mL) was added acryloyl chloride ((2.91 mL, 32.15 mmol) in THF (15.00 mL) dropwise at 0° C. under argon atmosphere. The reaction mixture was stirred for 10 min at 0° C. The resulting mixture was diluted with water (500 mL) and extracted with EA (3×500 mL). The combined organic layers were washed with brine (2×300 mL), dried over anhydrous Na₂SO₄, filtered and concentrated under reduced pressure. The residue was purified by silica gel Column chromatography, eluted with 5% DCM in MeOH. The fractions contained desired product were combined and concentrated to afford 3-ethynyl-1-[(3S,5R)-5-(methoxymethyl)-1-(prop-2-enoyl)pyrrolidin-3-yl]-5-(methylamino)pyrazole-4-carboxamide (11.1 g, 84%) as an off-white solid. MS ESI calculated for C₁₆H₂₁N₅O₃ [M+H]⁺, 332.16, found 332.20; ¹H NMR (400 MHz, CDCl₃) δ 6.76 (s, 1H), 6.60-6.36 (m, 2H), 5.74-5.68 (m, 1H), 5.50-5.20 (m, 2H), 4.55-4.39 (m, 1H), 4.06-3.83 (m, 3H), 3.53-3.40 (m, 2H), 3.36-3.35 (m, 3H), 3.03-2.99 (m, 3H), 2.68-2.60 (m, 1H), 2.37-2.23 (m, 1H).

### Step 11: 3-[2-(6-Chloro-1-ethyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a stirred mixture of 3-ethynyl-1-[(3S,5R)-5-(methoxymethyl)-1-(prop-2-enoyl)pyrrolidin-3-yl]-5-(methylamino)pyrazole-4-carboxamide (0.45 g, 1.36 mmol) and 6-chloro-1-ethyl-5-iodo-1,3-benzodiazole (0.50 g, 1.63 mmol) in DMF (5.00 mL) was added CuI (51.73 mg, 0.27 mmol), Pd(PPh₃)₂Cl₂ (95.32 mg, 0.14 mmol) and TEA (0.41 g, 4.07 mmol) dropwise at room temperature. The reaction mixture was degassed with argon for three times and stirred for 40 min at 80° C. The resulting mixture was concentrated under reduced pressure. The residue was purified by silica gel Column chromatography, eluted with CH₂Cl₂/MeOH (0-10%) to afford the crude product. The crude product was purified by reverse flash chromatography with the following conditions: Column: XBridge Prep C18 OBD Column, 5×25 cm 5 μm; Mobile Phase A: Water (10 mmol/L NH₄HCO₃), Mobile Phase B: ACN; Flow rate: 40 mL/min; Gradient: 5 B to 55 B in 60 min; 210/254 nm. The fractions contained desired product were combined and concentrated to afford 3-[2-(6-chloro-1-ethyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (0.35 g, 50%) as an off-white solid. MS ESI calculated for C₂₅H₂₈ClN₇O₃[M+H]⁺, 510.19, found 510.10; ¹H NMR (300 MHz, CDCl₃) δ 8.02-7.96 (m, 2H), 7.21 (s, 1H), 6.83 (d, J=6.2 Hz, 1H), 6.47-6.37 (m, 2H), 5.72 (dd, J=8.2, 4.2 Hz, 1H), 5.62-5.31 (m, 2H), 4.60-4.56 (m, 1H), 4.28-4.24 (m, 2H), 4.19-4.02 (m, 2H), 3.94-3.89 (m, 1H), 3.42-3.36 (m, 4H), 3.10-3.04 (m, 3H), 2.76-2.73 (m, 1H), 2.46-2.26 (m, 1H), 1.59-1.55 (m, 3H).

### Example 90: 3-[2-(6-Fluoro-1H-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

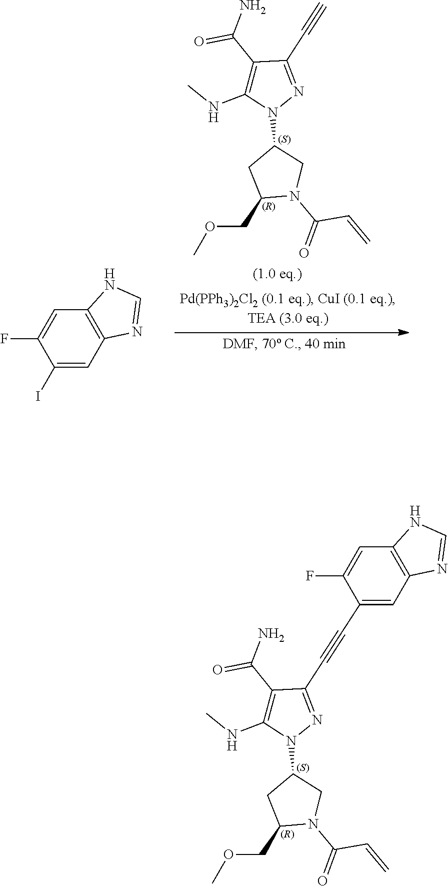

To a stirred mixture of 3-ethynyl-1-[(3S,5R)-5-(methoxymethyl)-1-(prop-2-enoyl)pyrrolidin-3-yl]-5-(methylamino)pyrazole-4-carboxamide (0.20 g, 0.60 mmol), 5-fluoro-6-iodo-3H-1,3-benzodiazole (0.19 g, 0.72 mmol), Pd(PPh₃)₂Cl₂ (42.36 mg, 0.06 mmol), CuI (22.99 mg, 0.12 mmol) in DMF (4.00 mL) was added TEA (0.18 g, 1.81 mmol) dropwise at room temperature. The reaction mixture was degassed with argon for three times and stirred for 40 min at 70° C. The resulting mixture was concentrated under reduced pressure. The residue was purified by silica gel Column chromatography, eluted with MeOH in DCM (0-5%) to afford the crude product. The crude product was purified by Prep-HPLC with the following conditions: Column: XBridge Prep C18 OBD Column, 19×150 mm 5 μm; Mobile Phase A: Water (10 mmol/L NH₄HCO₃), Mobile Phase B: ACN; Flow rate: 20 mL/min; Gradient: 5 B to 50 B in 5.8 min; 210/254 nm; RT: 5.75 min. The fractions contained desired product were combined and concentrated to afford 3-[2-(6-fluoro-1H-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (69.60 mg, 24%) as an off-white solid. MS ESI calculated for C₂₃H₂₄FN₇O₃ [M+H]⁺, 466.19, found 466.05; ¹H NMR (400 MHz, CDCl₃) δ 11.35 (d, J=184.5 Hz, 1H), 8.17 (s, 1H), 7.69 (d, J=73.3 Hz, 1H), 7.17 (s, 1H), 6.88-6.70 (m, 1H), 6.57-6.25 (m, 2H), 5.74 (d, J=10.9 Hz, 1H), 5.66-5.15 (m, 2H), 4.70-4.38 (m, 1H), 4.15-4.07 (m, 2H), 3.90 (d, J=9.2 Hz, 1H), 3.60-3.17 (m, 5H), 3.06-3.01 (m, 3H), 2.76-2.68 (m, 1H), 2.35-2.07 (m, 1H).

### Example 91: 3-[2-(6-Fluoro-2-methyl-1H-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

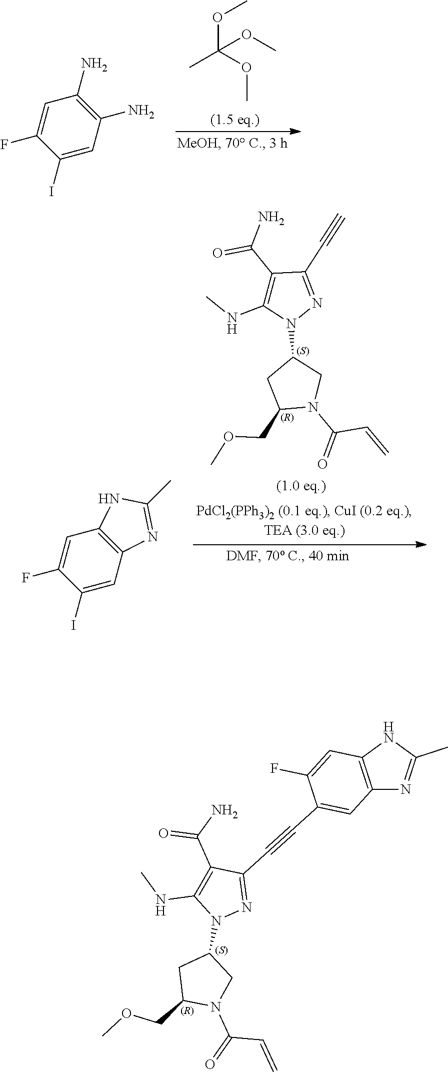

### Step 1:5-Fluoro-6-iodo-2-methyl-3H-1,3-benzodiazole

To a stirred solution of 4-fluoro-5-iodobenzene-1,2-diamine (5.00 g, 19.84 mmol) in MeOH (50.00 mL) was added 1,1,1-trimethoxyethane (3.58 g, 29.76 mmol) at room temperature. The reaction mixture was stirred for 3 h at 70° C. under argon atmosphere. The resulting mixture was cooled down and concentrated under reduced pressure. The residue was purified by silica gel Column chromatography, eluted with EA in PE (0-80%). The fractions contained desired product were combined and concentrated to afford 5-fluoro-6-iodo-2-methyl-3H-1,3-benzodiazole (4.96 g, 90%) as a brown semi-solid. MS ESI calculated for C₈H₆FN₂ [M+H]⁺, 276.96, found 276.85; ¹H NMR (400 MHZ, CDCl₃) δ 7.89 (d, J=5.3 Hz, 1H), 7.30 (s, 1H), 2.64 (s, 3H).

### Step 2:3-[2-(6-Fluoro-2-methyl-1H-1,3-benzodiazol-5-yl) ethynyl]-1-[(3S,5R)-5-(methoxymethyl)-1-(prop-2-enoyl)pyrrolidin-3-yl]-5-(methylamino)pyrazole-4-carboxamide

To a stirred solution of 3-ethynyl-1-[(3S,5R)-5-(methoxymethyl)-1-(prop-2-enoyl)pyrrolidin-3-yl]-5-(methylamino)pyrazole-4-carboxamide (0.20 g, 0.60 mmol), 5-fluoro-6-iodo-2-methyl-3H-1,3-benzodiazole (0.18 g, 0.66 mmol), Pd(PPh₃)₂Cl₂ (42.36 mg, 0.06 mmol) and CuI (22.99 mg, 0.12 mmol) in DMF (6.00 mL) was added TEA (0.18 g, 1.81 mmol) at room temperature under argon atmosphere. The reaction mixture was degassed with argon for three times and stirred for 40 minutes at 70° C. The resulting mixture was concentrated under reduced pressure. The residue was purified by silica gel Column chromatography, eluted with CH₂Cl₂/MeOH (10/1). The fractions contained desired product were combined and concentrated. The crude product was purified by reverse flash chromatography with the following conditions: Column: XBridge Prep C18 OBD Column, 19×150 mm 5 μm; Mobile Phase A: Water (10 mmol/L NH₄HCO₃), Mobile Phase B: ACN; Flow rate: 20 mL/min; Gradient: 5 B to 50 B in 5.8 min; 210/254 nm; RT: 5.58 min. The fractions contained desired product were combined and concentrated to afford 3-[2-(6-fluoro-2-methyl-1H-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (0.12 g, 40%) as an off-white solid. MS ESI calculated for C₂₄H₂₆FN₇O₃[M+H]⁺, 480.22, found 480.10; ¹H NMR (400 MHz, CDCl₃) δ 7.67-7.58 (m, 1H), 7.27 (d, J=23.5 Hz, 1H), 7.18 (s, 1H), 6.83-6.81 (m, 1H), 6.62-6.35 (m, 2H), 5.78-5.72 (m, 1H), 5.55-5.26 (m, 2H), 4.58-4.45 (m, 1H), 4.18-4.10 (m, 1H), 4.08-3.95 (m, 1H), 3.90-3.85 (m, 1H), 3.45-3.42 (m, 1H), 3.39-3.35 (m, 3H), 3.04-3.01 (m, 3H), 2.79-2.62 (m, 1H), 2.65 (s, 3H), 2.45-2.29 (m, 1H).

### Example 92: 3-[2-(6-Chloro-1H-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

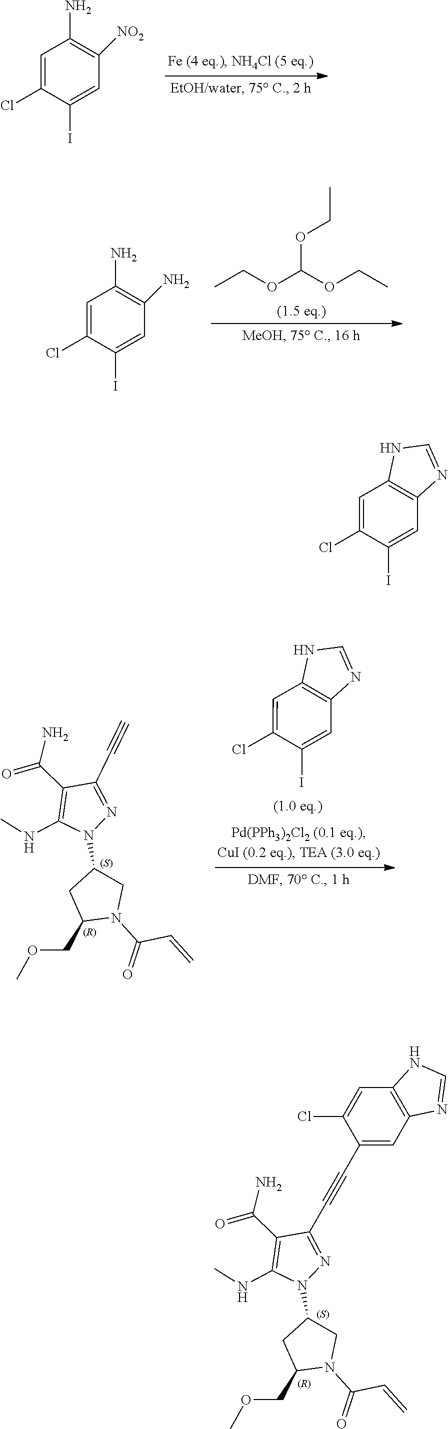

### Step 1: 4-Chloro-5-iodobenzene-1,2-diamine

To a stirred mixture of 5-chloro-4-iodo-2-nitroaniline (3.00 g, 10.05 mmol) and Fe (0.22 g, 4.01 mmol) in EtOH (10.50 mL) and H₂O (1.50 mL) was added NH₄Cl (0.27 g, 5.01 mmol) at room temperature. The reaction mixture was stirred for 2 h at 75° C. The resulting mixture was filtered, the filter cake was washed with EtOH (3×200 mL). The filtrate was concentrated under reduced pressure. The residue was diluted with water (300 mL) and extracted with EtOAc (3×200 mL). The combined organic layers were washed with brine (2×300 mL), dried over anhydrous Na₂SO₄ and filtered. The filtrate was concentrated under reduced pressure. The residue was purified by silica gel Column chromatography, eluted with 50% PE in EA. The fractions contained desired product were combined and concentrated to afford 4-chloro-5-iodobenzene-1,2-diamine (1.13 g, 41%) as a light yellow solid. MS ESI calculated for C₆H₆ClIN₂ [M+H]⁺, 268.93, found 268.90; ¹H NMR (400 MHz, CDCl₃) δ 7.11 (s, 1H), 6.80 (s, 1H).

### Step 2: 5-Chloro-6-iodo-3H-1,3-benzodiazole

To a stirred mixture of 4-chloro-5-iodobenzene-1,2-diamine (1.00 g, 3.73 mmol) in MeOH (15.00 mL) was added trimethyl orthoformate (0.59 g, 5.59 mmol). The reaction mixture was stirred for 16 h at 75° C., The resulting mixture was concentrated under reduced pressure. The residue was purified by silica gel Column chromatography, eluted with 5% MeOH in DCM. The fractions contained desired product were combined and concentrated to afford 5-chloro-6-iodo-3H-1,3-benzodiazole (0.66 g, 63%) as a light brown solid. MS ESI calculated for C₇H₄CₗIN₂[M+H]⁺, 278.91, found 278.95.

### Step 3: 3-[2-(6-Chloro-1H-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a stirred mixture of 3-ethynyl-1-[(3S,5R)-5-(methoxymethyl)-1-(prop-2-enoyl) pyrrolidin-3-yl]-5-(methylamino) pyrazole-4-carboxamide (0.24 g, 0.72 mmol), 5-chloro-6-iodo-3H-1,3-benzodiazole (0.20 g, 0.72 mmol), CuI (27.20 mg, 0.14 mmol) and Pd(PPh₃)₂Cl₂ (50.41 mg, 0.07 mmol) in DMF (2.00 mL) was added TEA (0.40 mL, 2.88 mmol) at room temperature. The reaction mixture was degassed with argon for three times and stirred for 1 h at 70° C. The resulting mixture was concentrated under reduced pressure. The residue was purified by silica gel Column chromatography, eluted with 6% MeOH in DCM. The fractions contained desired product were combined and concentrated. The crude product was purified by Prep-HPLC with the following conditions: Column: X Bridge Prep C18 OBD Column, 19×150 mm 5 μm; Mobile Phase A: Water (10 mmol/L NH₄HCO₃), Mobile Phase B: ACN; Flow rate: 20 mL/min; Gradient: 35 B to 70 B in 5.8 min, 210/254 nm; RT: 5.58 min. The fractions contained desired product were combined and concentrated to afford 3-[2-(6-chloro-1H-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (0.20 g, 57%) as a brown solid. MS ESI calculated for C₂₃H₂₄ClN₇O₃[M+H]⁺, 482.16, found 482.05; ¹H NMR (300 MHz, DMSO-d₆) δ 12.84 (s, 1H), 8.41 (s, 1H), 7.91 (d, J=37.6 Hz, 2H), 7.52 (s, 1H), 7.04-6.49 (m, 3H), 6.18 (d, J=16.6 Hz, 1H), 5.87-5.62 (m, 1H), 5.39-5.19 (m, 1H), 4.49-4.45 (m, 1H), 4.07-3.56 (m, 4H), 3.34-3.29 (m, 3H), 2.97-2.94 (m, 3H), 2.56-2.54 (m, 1H), 2.33-2.29 (m, 1H).

### Example 93: 3-[2-[6-Chloro-3-(difluoromethyl)-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide

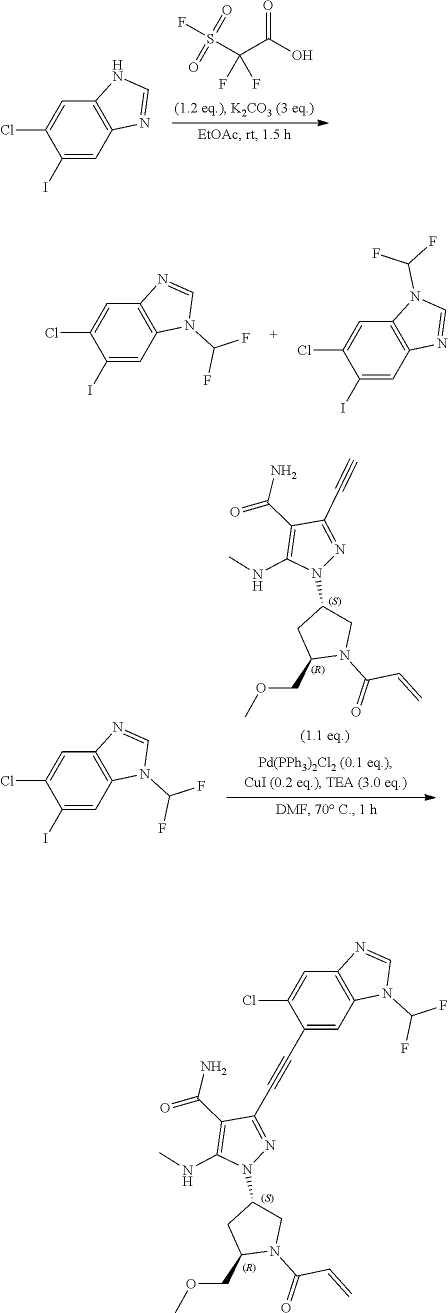

### Step 1: 5-Chloro-1-(difluoromethyl)-6-iodo-1,3-benzodiazole and 6-chloro-1-(difluoromethyl)-5-iodo-1,3-benzodiazole

To a stirred mixture of 5-chloro-6-iodo-3H-1,3-benzodiazole (3.20 g, 11.49 mmol) and K₂CO₃ (4.76 g, 34.47 mmol) in EA (32.00 mL) was added difluoro(sulfo)acetic acid (2.46 g, 13.79 mmol) dropwise at 0° C. under nitrogen atmosphere. The reaction mixture was stirred for 1.5 h at room temperature. The resulting mixture was concentrated under reduced pressure. The residue was purified by silica gel Column chromatography, eluted with 39% EA in PE. The fractions contained desired product were combined and concentrated. The crude product was purified by Prep-SFC with the following conditions Column: Chiralpak AD-H, 5×25 cm, 5 μm; Mobile Phase A: CO₂, Mobile Phase B: MeOH (0.1% 2 M NH₃-MeOH); Flow rate: 200 mL/min; Gradient: 50% B, 220 nm; RT1: 4.98 min; RT2: 6.49 min; Injection volume: 2.5 mL; Number of runs: 20. The faster peak contained desired product were combined and concentrated to afford 5-chloro-1-(difluoromethyl)-6-iodo-1,3-benzodiazole (0.97 g, 24%) as an off-white solid. MS ESI calculated for C₈H₄ClF₂IN₂ [M+H]⁺, 328.91, found 328.95; ¹H NMR (400 MHz, CDCl₃) δ 8.14 (s, 1H), 8.09 (s, 1H), 7.96 (s, 1H), 7.28 (t, J=60.1 Hz, 1H).

The slower peak contained desired product were combined and concentrated to afford 6-chloro-1-(difluoromethyl)-5-iodo-1,3-benzodiazole (0.99 g, 24%) as an off-white solid. MS ESI calculated for C₈H₄ClF₂IN₂ [M+H]⁺, 328.91, found 328.95; ¹H NMR (400 MHz, CDCl₃) δ 8.35 (s, 1H), 8.08 (s, 1H), 7.78 (s, 1H), 7.29 (t, J=60.1 Hz, 1H).

### Step 2: 3-[2-[6-Chloro-3-(difluoromethyl)-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide

To a stirred mixture of 5-chloro-1-(difluoromethyl)-6-iodo-1,3-benzodiazole (0.3 g, 0.91 mmol), 3-ethynyl-1-[(3S,5R)-5-(methoxymethyl)-1-(prop-2-enoyl)pyrrolidin-3-yl]-5-(methylamino)pyrazole-4-carboxamide (0.33 g, 1.01 mmol), CuI (34.79 mg, 0.18 mmol) and Pd(PPh₃)₂Cl₂ (64.10 mg, 0.09 mmol) in DMF (6.00 mL) was added TEA (0.38 mL, 3.76 mmol). The reaction mixture was degassed with argon for three times and stirred for 1 h at 70° C. The resulting mixture was diluted with EtOAc (300 mL), washed with water (3×100 mL), dried over anhydrous Na₂SO₄ and filtered. The filtrate was concentrated under reduced pressure. The residue was purified by Prep-HPLC with the following conditions: Column: XBridge Prep C18 OBD Column, 19×150 mm 5 μm; Mobile Phase A: Water (10 mmol/L NH₄HCO₃), Mobile Phase B: ACN; Flow rate: 20 mL/min; Gradient: 35 B to 70 B in 5.8 min; 210 nm; RT: 5.58 min. The fractions contained desired product were combined and concentrated to afford 3-[2-[6-chloro-3-(difluoromethyl)-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide (0.30 g, 62%) as an off-white solid. MS ESI calculated for C₂₄H₂₄ClF₂N₇O₃ [M+H]⁺, 532.16, found 532.20; ¹H NMR (400 MHz, CDCl₃) δ 8.22 (s, 1H), 7.98 (d, J=8.0 Hz, 1H), 7.93 (d, J=2.0 Hz, 1H), 7.34 (s, 1H), 7.16 (s, 1H), 6.59-6.36 (m, 2H), 5.78-5.70 (m, 1H), 5.61-5.26 (m, 2H), 4.65-4.35 (m, 1H), 4.15-3.96 (m, 2H), 3.94-3.83 (m, 1H), 3.55-3.42 (m, 1H), 3.39 (d, J=5.6 Hz, 3H), 3.05 (d, J=14.2 Hz, 3H), 2.78-2.66 (m, 1H), 2.45-2.29 (m, 1H).

### Example 94: 3-[2-[6-Chloro-1-(difluoromethyl)-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide

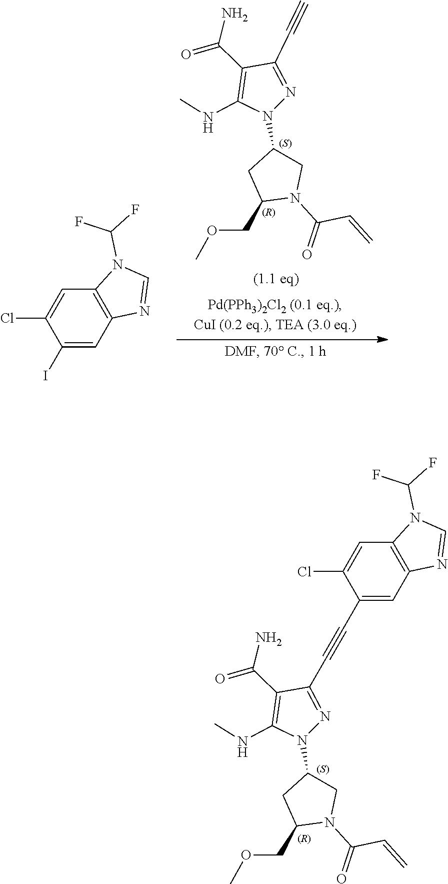

To a stirred mixture of 6-chloro-1-(difluoromethyl)-5-iodo-1,3-benzodiazole (0.3 g, 0.91 mmol), 3-ethynyl-1-[(3S,5R)-5-(methoxymethyl)-1-(prop-2-enoyl)pyrrolidin-3-yl]-5-(methylamino)pyrazole-4-carboxamide (0.33 g, 1.01 mmol), CuI (34.79 mg, 0.18 mmol) and Pd(PPh₃)₂Cl₂ (64.10 mg, 0.09 mmol) in DMF (6.00 mL) was added TEA (0.38 mL, 3.76 mmol). The reaction mixture was degassed with argon for three times and stirred for 1 h at 70° C. The resulting mixture was diluted with EtOAc (300 mL), washed with water (3×100 mL), dried over anhydrous Na₂SO₄ and filtered. The filtrate was concentrated under reduced pressure. The residue was purified by Prep-HPLC with the following conditions: Column: Atlantis HILIC OBD Column, 19×150 mm×5 μm; Mobile Phase A: Water (10 mmol/L NH₄HCO₃), Mobile Phase B: ACN; Flow rate: 20 mL/min; Gradient: 35 B to 70 B in 5.8 min; 210/254 nm. The fractions contained desired product were combined and concentrated to afford 3-[2-[6-chloro-1-(difluoromethyl)-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide (0.23 g, 47%) as an off-white solid. MS ESI calculated for C₂₄H₂₄ClF₂N₇O₃ [M+H]⁺, 532.16 found 532.20; ¹H NMR (400 MHz, CDCl₃) δ 8.33-8.09 (m, 2H), 7.79 (s, 1H), 7.50-7.31 (m, 1H), 7.23-7.06 (m, 1H), 6.57-6.38 (m, 2H), 5.77-5.70 (m, 1H), 5.61-5.25 (m, 2H), 4.63-4.39 (m, 1H), 4.17-4.00 (m, 2H), 3.95-3.85 (m, 1H), 3.55-3.43 (m, 1H), 3.40-3.39 (m, 3H), 3.07-3.04 (m, 3H), 2.79-2.67 (m, 1H), 2.45-2.28 (m, 1H).

### Example 95: 3-[2-[6-Chloro-3-(difluoromethyl)-2-methyl-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide

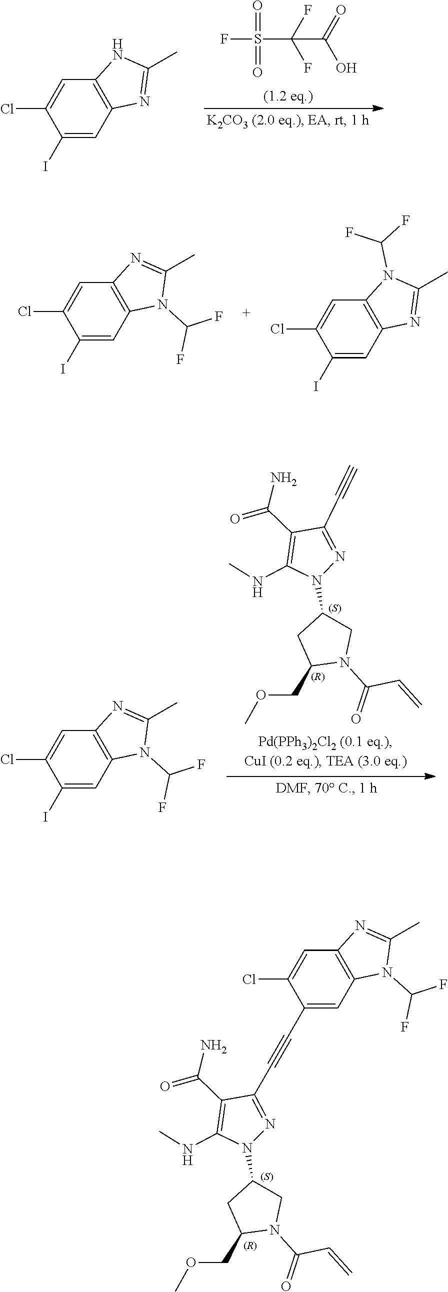

### Step 1: 5-Chloro-1-(difluoromethyl)-6-iodo-2-methyl-1H-benzo[d]imidazole and 6-chloro-1-(difluoromethyl)-5-iodo-2-methyl-1H-benzo[d]imidazole

To a stirred mixture of 5-chloro-6-iodo-2-methyl-3H-1,3-benzodiazole (0.30 g, 1.03 mmol) and K₂CO₃ (0.28 g, 2.05 mmol) in EtOAc (5.00 mL) was added difluoro(sulfo)acetic acid (0.22 g, 1.23 mmol) at 0° C. The reaction mixture was stirred for 1 h at room temperature. The resulting mixture was concentrated under reduced pressure. The residue was purified by silica gel Column chromatography, eluted with CH₂Cl₂/MeOH (10/1) to afford the crude product. The crude product (1.8 g) was purified by Prep-SFC with the following conditions Column: Chiralpak IG, 5×25 cm, 10 μm; Mobile Phase A: CO₂, Mobile Phase B: MeOH (0.1% 2 M NH₃-MeOH); Flow rate: 200 mL/min; Gradient: 50% B; 220 nm; RT1: 4.1 min; RT2: 9.07 min; Injection volume: 4.8 mL; Number of runs: 10. The faster peak contained desired product were combined and concentrated to afford 6-chloro-1-(difluoromethyl)-5-iodo-2-methyl-1H-benzo[d]imidazole (0.82 g, 35%) as a white solid. ¹H NMR (300 MHz, CDCl₃) δ 8.19 (s, 1H), 7.72 (s, 1H), 7.48-7.00 (m, 1H), 2.71 (s, 3H).

The slower peak contained desired product were combined and concentrated to afford 5-chloro-1-(difluoromethyl)-6-iodo-2-methyl-1H-benzo[d]imidazole (0.85 g, 36%) as a white solid. ¹H NMR (300 MHz, CDCl₃) δ 8.07 (s, 1H), 7.82 (s, 1H), 7.45-6.95 (m, 1H), 2.71 (s, 3H).

### Step 2: 3-[2-[6-Chloro-3-(difluoromethyl)-2-methyl-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide

To a stirred mixture of 5-chloro-1-(difluoromethyl)-6-iodo-2-methyl-1,3-benzodiazole (0.3 g, 0.88 mmol), 3-ethynyl-1-[(3S,5R)-5-(methoxymethyl)-1-(prop-2-enoyl)pyrrolidin-3-yl]-5-(methylamino)pyrazole-4-carboxamide (0.32 g, 0.96 mmol), CuI (33.36 mg, 0.18 mmol) and Pd(PPh₃)₂Cl₂ (61.48 mg, 0.09 mmol) in DMF (6.00 mL) was added TEA (0.37 mL, 3.61 mmol). The reaction mixture was degassed with argon for three times and stirred for 1 h at 70° C. The resulting mixture was diluted with EtOAc (300 mL), washed with water (3×100 mL), dried over anhydrous Na₂SO₄ and filtered. The filtrate was concentrated under reduced pressure. The residue was purified by Prep-HPLC with the following conditions: Column: XBridge Prep C18 OBD Column, 19×150 mm×5 μm; Mobile Phase A: Water (10 mmol/L NH₄HCO₃), Mobile Phase B: ACN; Flow rate: 20 mL/min; Gradient: 25 B to 60 B in 5.8 min; 210 nm; RT: 5.58 min; The fractions contained desired product were combined and concentrated to afford 3-[2-[6-chloro-3-(difluoromethyl)-2-methyl-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide (0.27 g, 56%) as an off-white solid. MS ESI calculated for C₂₅H₂₆ClF₂N₇O₃ [M+H]⁺, 546.18, found 546.25; ¹H NMR (400 MHz, CDCl₃) δ 7.91 (d, J=8.0 Hz, 1H), 7.78 (d, J=1.6 Hz, 1H), 7.16 (d, J=17.2 Hz, 1H), 6.59-6.36 (m, 2H), 5.78-5.70 (m, 1H), 5.60-5.20 (m, 2H), 4.62-4.38 (m, 1H), 4.15-3.96 (m, 2H), 3.94-3.86 (m, 1H), 3.55-3.42 (m, 1H), 3.39 (d, J=5.2 Hz, 3H), 3.05 (d, J=14.4 Hz, 3H), 2.75 (s, 3H), 2.78-2.66 (m, 1H), 2.45-2.28 (m, 1H).

### Example 96: 3-[2-[6-Chloro-1-(difluoromethyl)-2-methyl-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide

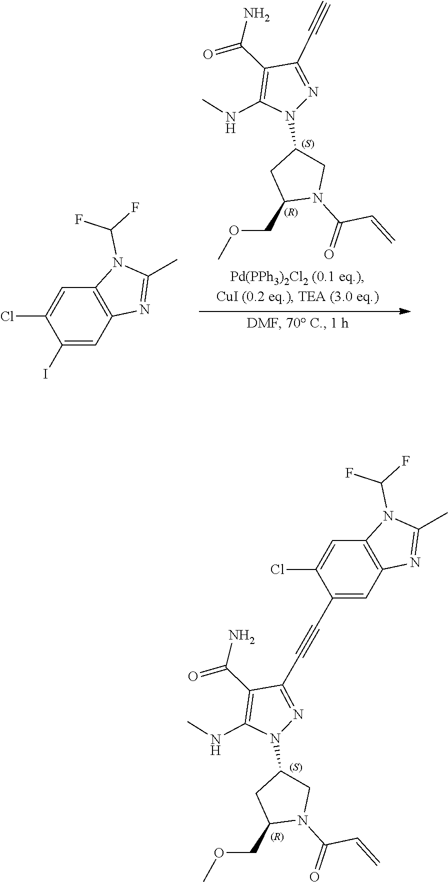

To a stirred mixture of 6-chloro-1-(difluoromethyl)-5-iodo-2-methyl-1,3-benzodiazole (0.3 g, 0.88 mmol), 3-ethynyl-1-[(3S,5R)-5-(methoxymethyl)-1-(prop-2-enoyl)pyrrolidin-3-yl]-5-(methylamino)pyrazole-4-carboxamide (0.32 g, 0.96 mmol), CuI (33.36 mg, 0.18 mmol) and Pd(PPh₃)₂Cl₂ (61.48 mg, 0.09 mmol) in DMF (6.00 mL) was added TEA (0.37 mL, 3.61 mmol). The reaction mixture was degassed with argon for three times and stirred for 1 h at 70° C. The resulting mixture was diluted with EtOAc (300 mL), washed with water (3×100 mL), dried over anhydrous Na₂SO₄ and filtered. The filtrate was concentrated under reduced pressure. The residue was purified by Prep-HPLC with the following conditions: Column: XBridge Prep C18 OBD Column, 19×150 mm×5 μm; Mobile Phase A: Water (10 mmol/L NH₄HCO₃), Mobile Phase B: ACN; Flow rate: 20 mL/min; Gradient: 25 B to 60 B in 5.8 min; 210/254 nm; RT: 5.58 min. The fractions contained desired product were combined and concentrated to afford 3-[2-[6-chloro-1-(difluoromethyl)-2-methyl-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide (0.25 g, 52%) as an off-white solid. MS ESI calculated for C₂₅H₂₆ClF₂N₇O₃ [M+H]⁺, 546.18, found 546.25; ¹H NMR (400 MHz, CDCl₃) δ 7.99 (d, J=5.2 Hz, 1H), 7.69 (s, 1H), 7.14 (s, 1H), 6.59-6.36 (m, 2H), 5.77-5.69 (m, 1H), 5.59-5.26 (m, 2H), 4.63-4.36 (m, 1H), 4.17-3.99 (m, 2H), 3.95-3.85 (m, 1H), 3.55-3.42 (m, 1H), 3.39 (d, J=5.2 Hz, 3H), 3.05 (d, J=15.2 Hz, 3H), 2.74 (s, 3H), 2.74-2.66 (m, 1H), 2.44-2.28 (m, 1H).

### Example 97: 3-[2-(4,6-Difluoro-1-methyl-1,2,3-benzotriazol-5-yl)ethynyl]-1-[(3S,5R)-5-(methoxymethyl)-1-(prop-2-enoyl)pyrrolidin-3-yl]-5-(methylamino)pyrazole-4-carboxamide

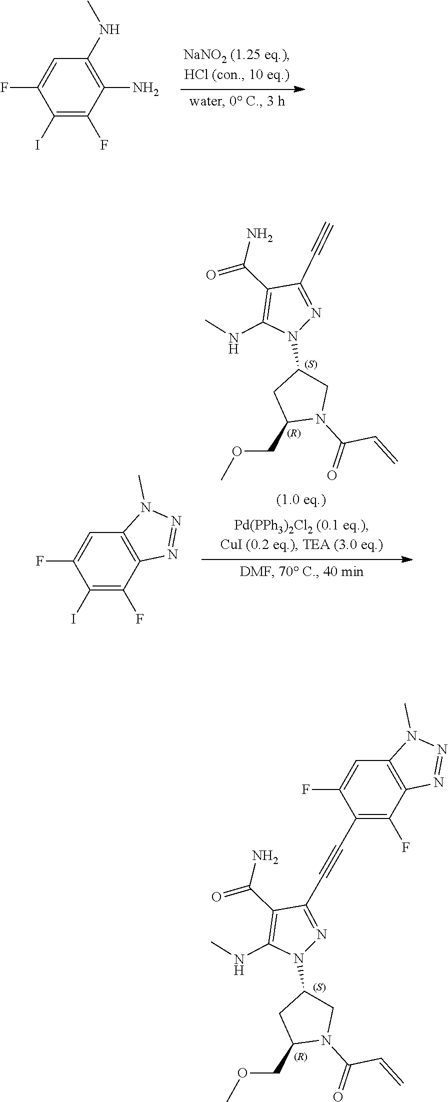

### Step 1: 4,6-Difluoro-5-iodo-1-methyl-1H-benzo[d][1,2,3]triazole

To a stirred solution of 3,5-difluoro-4-iodo-N¹-methylbenzene-1,2-diamine (0.54 g, 1.90 mmol) in con. HCl (1.58 mL, 19.01 mmol) and H₂O (6.32 mL) was added NaNO₂ (0.16 g, 2.39 mmol) in water (2 mL) dropwise at 0° C. The reaction mixture was stirred for 3 h at 0° C. The resulting mixture was neutralized to pH 7 with saturated KOH (0.5 M). The resulting mixture was washed with water (3×5 mL). The precipitated solids were collected by filtration, washed with water (3×10 mL) and dried to afford 4,6-difluoro-5-iodo-1-methyl-1H-benzo[d][1,2,3]triazole (0.36 g, 64%) as a brown solid. MS ESI calculated for C₇H₄F₂IN₃ [M+H]⁺, 295.94, found 296.00; ¹H NMR (400 MHz, CDCl₃) δ 7.11 (s, 1H), 4.31 (s, 3H).

### Step 2: 3-[2-(4,6-Difluoro-1-methyl-1,2,3-benzotriazol-5-yl)ethynyl]-1-[(3S,5R)-5-(methoxymethyl)-1-(prop-2-enoyl)pyrrolidin-3-yl]-5-(methylamino)pyrazole-4-carboxamide

To a stirred solution of 4,6-difluoro-5-iodo-1-methyl-1H-benzo[d][1,2,3]triazole (0.2 g, 0.67 mmol) and 3-ethynyl-1-[(3S,5R)-5-(methoxymethyl)-1-(prop-2-enoyl)pyrrolidin-3-yl]-5-(methylamino)pyrazole-4-carboxamide (0.18 g, 0.56 mmol) in DMF (2.00 mL, 25.84 mmol) were added Pd(PPh₃)₂Cl₂ (47.58 mg, 0.07 mmol), CuI (25.82 mg, 0.13 mmol) and TEA (0.20 g, 2.03 mmol) dropwise at room temperature. The reaction mixture was degassed with argon for three times and stirred for 40 min at 70° C. The resulting mixture was concentrated under reduced pressure. The residue was purified by silica gel Column chromatography, eluted with CH₂Cl₂/MeOH (0-10%) to afford the crude. The crude product was purified by reverse flash chromatography with the following conditions: Column: XBridge Prep C18 OBD Column, 19×150 mm 5 μm; Mobile Phase A: Water (10 mmol/L NH₄HCO₃), Mobile Phase B: ACN; Flow rate: 40 mL/min; Gradient: 5 B to 35 B in 30 min; 210/254 nm. The fractions contained desired product were combined and concentrated to afford 3-[2-(4,6-difluoro-1-methyl-1,2,3-benzotriazol-5-yl)ethynyl]-1-[(3S,5R)-5-(methoxymethyl)-1-(prop-2-enoyl)pyrrolidin-3-yl]-5-(methylamino)pyrazole-4-carboxamide (55.5 mg, 16%) as a white solid. MS ESI calculated for C₂₃H₂₄F₂N₈O₃[M+H]⁺, 499.19, found 499.10; ¹H NMR (300 MHz, CDCl₃) δ 7.13 (d, J=7.4 Hz, 1H), 6.45 (d, J=8.8 Hz, 2H), 5.73 (m, 1H), 5.59-5.22 (m, 2H), 4.58-4.45 (m, 1H), 4.32 (s, 3H), 4.11-4.09 (m, 2H), 3.92-3.89 (m, 1H), 3.50-3.48 (m, 1H), 3.39 (d, J=3.9 Hz, 3H), 3.06 (d, J=11.3 Hz, 3H), 2.72-3.69 (m, 1H), 2.33-2.30 (m, 1H).

### Example 98: 3-[2-(6-Chloro-2-methyl-1H-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

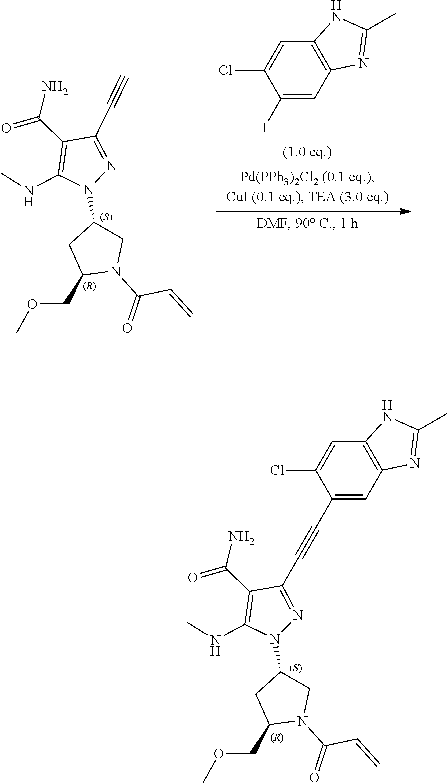

To a stirred solution of 3-ethynyl-1-[(3S,5R)-5-(methoxymethyl)-1-(prop-2-enoyl)pyrrolidin-3-yl]-5-(methylamino)pyrazole-4-carboxamide (0.15 g, 0.45 mmol), 5-chloro-6-iodo-2-methyl-3H-1,3-benzodiazole (0.13 g, 0.45 mmol), Pd(PPh₃)₂Cl₂ (31.77 mg, 0.05 mmol) and CuI (17.24 mg, 0.09 mmol) in DMF (0.50 mL) was added TEA (0.14 g, 1.36 mmol) dropwise at room temperature. The reaction mixture was degassed with argon for three times and stirred for 1 h at 90° C. The resulting mixture was concentrated under reduced pressure. The residue was purified by silica gel Column chromatography, eluted with CH₂Cl₂/MeOH (10/1). The fractions contained desired product were combined and concentrated. The crude product was purified by Prep-HPLC with the following conditions: Column: XBridge Prep C18 OBD Column, 19×150 mm 5 μm; Mobile Phase A: Water (10 mmol/L NH₄HCO₃), Mobile Phase B: ACN; Flow rate: 20 mL/min; Gradient: 30 B to 60 B in 5.8 min; 210/254 nm; RT: 5.8 min. The fractions contained desired product were combined and concentrated to afford 3-[2-(6-chloro-2-methyl-1H-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (0.11 g, 50%) as a white solid. MS ESI calculated for C₂₄H₂₆ClN₇O₃[M+H]⁺, 496.20, found 496.10; ¹H NMR (400 MHz, DMSO-d₆) δ 12.67 (s, 1H), 7.82 (s, 1H), 7.70 (s, 1H), 7.53 (s, 1H), 6.86 (s, 1H), 6.76-6.46 (m, 2H), 6.17-5.86 (m, 1H), 5.70-5.46 (m, 1H), 5.28-4.96 (m, 1H), 4.59-4.34 (m, 1H), 4.06-3.74 (m, 2H), 3.65-3.40 (m, 2H), 3.31 (s, 3H), 2.96 (t, J=5.1 Hz, 3H), 2.66-2.57 (m, 1H), 2.51 (s, 3H), 2.36-2.27 (m, 1H).

### Example 99: 3-[2-(6-Chloro-1-ethyl-4-fluoro-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

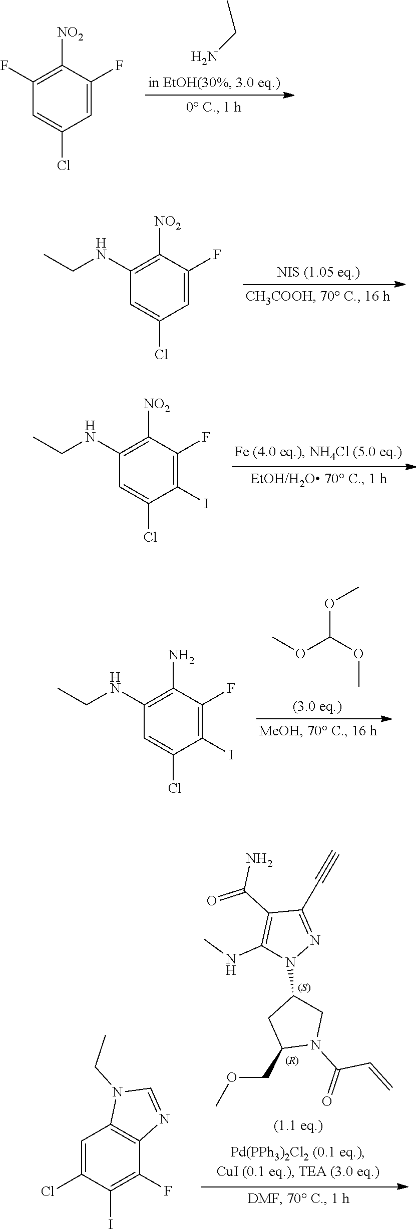

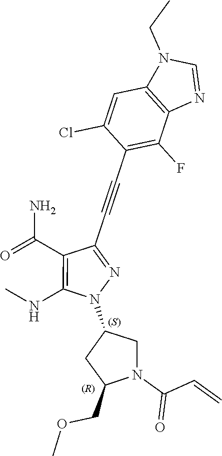

### Step 1: 5-Chloro-N-ethyl-3-fluoro-2-nitroaniline

To a stirred solution of 5-chloro-1,3-difluoro-2-nitrobenzene (4.50 g, 23.25 mmol) in EtOH (90.00 mL) was added ethylamine in EtOH (13.27 mL, 294.33 mmol, 30%) dropwise at 0° C. under argon atmosphere. The reaction mixture was stirred for additional 1 h at 0° C. The resulting mixture was diluted with water (100 mL) and filtered, the filter cake was washed with water (3×100 mL). The filter cake was dried to afford 5-chloro-N-ethyl-3-fluoro-2-nitroaniline (5.0 g, 98%) as a red solid which was used in the next step directly without further purification. MS ESI calculated for C₈H₈ClFN₂O₂[M+H]⁺, 219.03, found 219.10.

### Step 2: 5-Chloro-N-ethyl-3-fluoro-4-iodo-2-nitroaniline

To a stirred solution of 5-chloro-N-ethyl-3-fluoro-2-nitroaniline (5.00 g, 22.87 mmol) in AcOH (100.00 mL) was added and NIS (5.40 g, 24.00 mmol). The reaction mixture was stirred for 16 h at 70° C. under argon atmosphere. The resulting mixture was concentrated under reduced pressure. The residue was neutralized to pH 7 with saturated NaHCO₃ (aq.). The resulting mixture was extracted with EtOAc (3×500 mL). The combined organic layers were washed with water (3×200 mL), dried over anhydrous Na₂SO₄ and filtered. The filtrate was concentrated under reduced pressure. The residue was purified by silica gel Column chromatography, eluted with 15% EA in PE. The fractions contained desired product were combined and concentrated to afford 5-chloro-N-ethyl-3-fluoro-4-iodo-2-nitroaniline (7.5 g, 95%) as a red solid. MS ESI calculated for C₈H₇ClFIN₂O₂[M−H]⁻, 342.92, found 342.90; ¹H NMR (400 MHz, CDCl₃) δ 7.12 (s, 1H), 6.85 (d, J=2.0 Hz, 1H), 3.35-3.23 (m, 2H), 1.37 (t, J=7.2 Hz, 3H).

### Step 3: 5-Chloro-N1-ethyl-3-fluoro-4-iodobenzene-1,2-diamine

To a stirred mixture of 5-chloro-N-ethyl-3-fluoro-4-iodo-2-nitroaniline (7.30 g, 21.19 mmol) and NH₄Cl (5.67 g, 106.00 mmol) in EtOH (80.00 mL) and H₂O (20.00 mL) was added Fe (4.73 g, 84.70 mmol). The reaction mixture was stirred for 1 h at 70° C. under argon atmosphere. The resulting mixture was filtered, the filter cake was washed with EtOAc (3×400 mL). The combined organic layers were washed with water (4×200 mL), dried over anhydrous Na₂SO₄ and filtered. The filtrate was concentrated under reduced pressure to afford 5-chloro-N¹-ethyl-3-fluoro-4-iodobenzene-1,2-diamine (6.8 g, 96%) as a red oil which was used in the next step directly without further purification. MS ESI calculated for C₈H₉ClFIN₂ [M+H]⁺, 314.95, found 314.90.

### Step 4: 6-Chloro-1-ethyl-4-fluoro-5-iodo-1,3-benzodiazole

To a stirred solution of 5-chloro-N¹-ethyl-3-fluoro-4-iodobenzene-1,2-diamine (3.00 g, 9.54 mmol) in MeOH (60.00 mL) was added trimethyl orthoformate (3.13 mL, 28.61 mmol). The reaction mixture was stirred for 16 h at 70° C. under argon atmosphere. The resulting mixture was concentrated under reduced pressure. The residue was purified by silica gel Column chromatography, eluted with PE/EtOAc (1/2). The fractions contained desired product were combined and concentrated to afford 6-chloro-1-ethyl-4-fluoro-5-iodo-1,3-benzodiazole (2.3 g, 74%) as a green solid. MS ESI calculated for C₉H₇ClFIN₂ [M+H]⁺, 324.93, found 324.95; ¹H NMR (400 MHz, CDCl₃) δ 7.91 (s, 1H), 7.44 (d, J=1.2 Hz, 1H), 4.22 (q, J=7.2 Hz, 2H), 1.57 (t, J=7.2 Hz, 3H).

### Step 5: 3-[2-(6-Chloro-1-ethyl-4-fluoro-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a stirred mixture of 6-chloro-1-ethyl-4-fluoro-5-iodo-1,3-benzodiazole (0.3 g, 0.92 mmol), 3-ethynyl-1-[(3S,5R)-5-(methoxymethyl)-1-(prop-2-enoyl)pyrrolidin-3-yl]-5-(methylamino)pyrazole-4-carboxamide (0.34 g, 1.02 mmol), CuI (35.21 mg, 0.19 mmol) and Pd(PPh₃)₂Cl₂ (64.89 mg, 0.09 mmol) in DMF (6.00 mL) was added TEA (0.39 mL, 3.81 mmol). The reaction mixture was degassed with argon for three times and stirred for 1 h at 70° C. The resulting mixture was diluted with EtOAc (300 mL), washed with water (3×100 mL), dried over anhydrous Na₂SO₄ and filtered. The filtrate was concentrated under reduced pressure. The residue was purified by Prep-HPLC with the following conditions: Column: XBridge Prep C18 OBD Column, 19×150 mm 5 μm; Mobile Phase A: Water (10 mmol/L NH₄HCO₃), Mobile Phase B: ACN; Flow rate: 20 mL/min; Gradient: 15 B to 50 B in 6 min; 210/254 nm; RT: 5.75 min. The fractions contained desired product were combined and concentrated to afford 3-[2-(6-chloro-1-ethyl-4-fluoro-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (0.20 g, 42%) as an off-white solid. MS ESI calculated for C₂₅H₂₇ClFN₇O₃[M+H]⁺, 528.18, found 528.25; ¹H NMR (400 MHz, CDCl₃) δ 7.98 (s, 1H), 7.35 (s, 1H), 7.14 (s, 1H), 6.57-6.36 (m, 2H), 5.82-5.67 (m, 1H), 5.56-5.25 (m, 2H), 4.63-4.39 (m, 1H), 4.31-4.19 (m, 2H), 4.19-4.07 (m, 1H), 4.07-3.97 (m, 1H), 4.00-3.87 (m, 1H), 3.55-3.42 (m, 1H), 3.39 (d, J=5.2 Hz, 3H), 3.04 (d, J=15.6 Hz, 3H), 2.79-2.67 (m, 1H), 2.43-2.26 (m, 1H), 1.58 (t, J=7.2 Hz, 3H).

### Example 100: 3-[2-(6-Chloro-4-fluoro-1-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

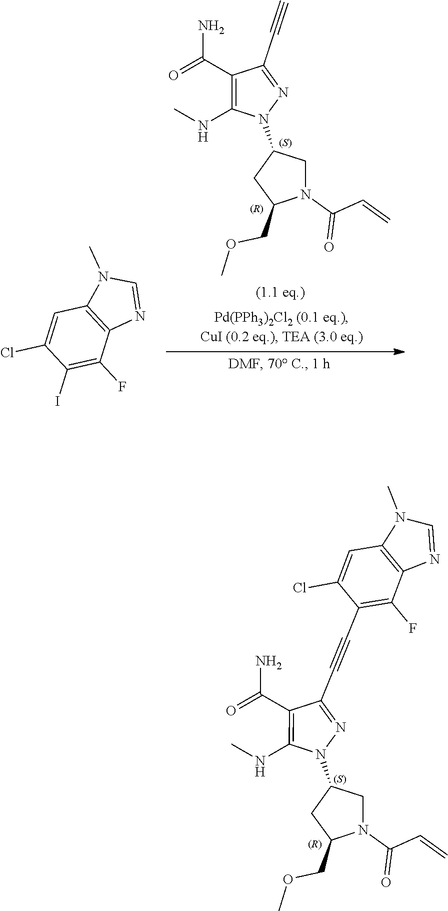

To a stirred mixture of 6-chloro-4-fluoro-5-iodo-1-methyl-1,3-benzodiazole (0.5 g, 1.13 mmol, 70%), 3-ethynyl-1-[(3S,5R)-5-(methoxymethyl)-1-(prop-2-enoyl)pyrrolidin-3-yl]-5-(methylamino)pyrazole-4-carboxamide (0.41 g, 1.24 mmol), CuI (42.94 mg, 0.23 mmol) and Pd(PPh₃)₂Cl₂ (79.12 mg, 0.11 mmol) in DMF (5.00 mL) was added TEA (0.50 mL, 4.94 mmol). The reaction mixture was degassed with argon for three times and stirred for 1 h at 70° C. The resulting mixture was diluted with EtOAc (300 mL), washed with water (3×100 mL), dried over anhydrous Na₂SO₄ and filtered. The filtrate was concentrated under reduced pressure. The residue was purified by Prep-HPLC with the following conditions Column: SunFire Prep C18 OBD Column, 19×150 mm×5 μm 10 nm; Mobile Phase A: Water (0.1% FA), Mobile Phase B: ACN; Flow rate: 20 mL/min; Gradient: 25 B to 45 B in 8 min; 254/210 nm; RT: 6.5 min. The fractions contained desired product were combined and concentrated to afford 3-[2-(6-chloro-4-fluoro-1-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (0.20 g, 34%) as an off-white solid. MS ESI calculated for C₂₄H₂₅ClFN₇O₃[M+H]⁺, 514.17, found 514.20; ¹H NMR (400 MHz, CDCl₃) δ 8.07 (s, 1H), 7.43 (s, 1H), 7.11 (s, 1H), 6.80 (s, 1H), 6.46 (d, J=11.2 Hz, 2H), 5.73 (d, J=9.2 Hz, 1H), 5.61-5.19 (m, 2H), 4.66-4.37 (m, 1H), 4.20-3.99 (m, 2H), 3.92 (s, 4H), 3.55-3.43 (m, 1H), 3.39 (d, J=4.8 Hz, 3H), 3.06 (d, J−15.6 Hz, 3H), 2.77-2.67 (m, 1H), 2.39-2.30 (m, 1H).

### Example 101: 3-[2-(6,7-Difluoro-1-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

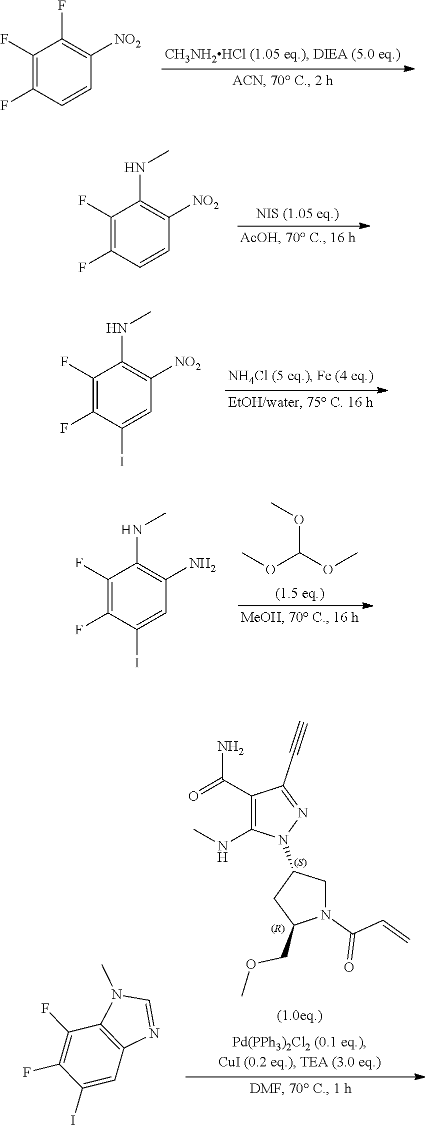

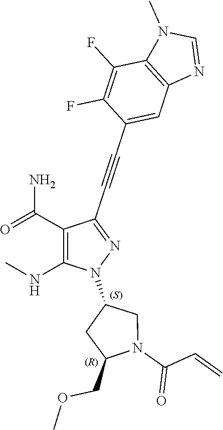

### Step 1: 2,3-Difluoro-N-methyl-6-nitroaniline

To a stirred mixture of 1,2,3-trifluoro-4-nitrobenzene (3.00 g, 16.94 mmol) in ACN (30.00 mL) was added CH₃NH₂HCl (1.20 g, 17.78 mmol) and DIEA (14.75 mL) at rt. The reaction mixture was stirred for 2 h at 70° C. under nitrogen atmosphere. The resulting mixture was concentrated under reduced pressure. The residue was diluted with water (100 mL) and extracted with EA (3×100 mL). The combined organic layers were washed with brine (2×150 mL), dried over anhydrous Na₂SO₄ and filtered. The filtrate was concentrated under reduced pressure to afford 2,3-difluoro-N-methyl-6-nitroaniline (3 g, 94%) as a light yellow solid which was used in the next step directly without further purification. ¹H NMR (300 MHz, CDCl₃) δ 7.99-7.95 (m, 1H), 6.46-6.44 (m, 1H), 3.29 (d, J=7.5 Hz, 3H).

### Step 2: 2,3-Difluoro-4-iodo-N-methyl-6-nitroaniline

To a stirred mixture of 2,3-difluoro-N-methyl-6-nitroaniline (3.23 g, 17.16 mmol) in AcOH (30.00 mL) was added and NIS (4.06 g, 18.04 mmol). The reaction mixture was stirred for 16 h at 70° C. under nitrogen atmosphere. The resulting mixture was concentrated under reduce pressure. The residue was neutralized to pH 7 with saturated NaHCO₃ (aq.). The resulting mixture was diluted with water (150 mL) and extracted with EA (3×150 mL). The combined organic layers were washed with brine (2×150 mL), dried over anhydrous Na₂SO₄ and filtered. The filtrate was concentrated under reduced pressure. The residue was purified by silica gel Column chromatography, eluted with 25% DCM in PE. The fractions contained desired product were combined and concentrated to afford 2,3-difluoro-4-iodo-N-methyl-6-nitroaniline (3.66 g, 67%) as a yellow solid. MS ESI calculated for C₇H₅F₂IN₂O₂[M+H]⁺, 315.03, found 315.00; ¹H NMR (300 MHz, CDCl₃) δ 8.38 (dd, J=6.3, 2.4 Hz, 1H), 7.98 (s, 1H), 3.29 (dd, J=7.5, 5.5 Hz, 3H).

### Step 3: 5,6-Difluoro-4-iodo-N1-methylbenzene-1,2-diamine

To a stirred mixture of 2,3-difluoro-4-iodo-N-methyl-6-nitroaniline (0.50 g, 1.59 mmol) and NH₄Cl (0.43 g, 7.96 mmol) in EtOH (8.75 ml) and water (1.25 ml) was added Fe (0.36 g, 6.36 mmol). The reaction mixture was stirred for 16 h at 75° C. The resulting mixture was cooled down and filtered, the filter cake was washed with EtOH (3×150 mL). The filtrate was concentrated under reduced pressure. The residue was diluted with water (100 mL) and extracted with EA (3×100 mL). The combined organic layers were washed with brine (150 mL), dried over anhydrous Na₂SO₄ and filtered. The filtrate was concentrated under reduced pressure to afford 5,6-difluoro-4-iodo-N¹-methylbenzene-1,2-diamine (0.22 g, 48%) as a yellow solid which was used in the next step directly without further purification. MS ESI calculated for C₇H₇F₂IN₂ [M+H]⁺, 284.96, found 285.00.

### Step 4: 6,7-Difluoro-5-iodo-1-methyl-1,3-benzodiazole

To a stirred solution of 5,6-difluoro-4-iodo-N¹-methylbenzene-1,2-diamine (0.22 g, 0.77 mmol) in MeOH (2.20 mL) was added trimethyl orthoformate (0.12 g, 1.16 mmol). The reaction mixture was stirred for 16 h at 70° C. under argon atmosphere. The resulting mixture was concentrated under reduce pressure. The residue was purified by silica gel Column chromatography, eluted with 8% MeOH in DCM. The fractions contained desired product were combined and concentrated to afford 6,7-difluoro-5-iodo-1-methyl-1,3-benzodiazole (0.20 g, 87%) as a light brown solid. MS ESI calculated for C₈H₅F₂IN₂ [M+H]⁺, 295.04, found 294.95; ¹H NMR (300 MHz, CDCl₃) δ 8.44 (s, 1H), 8.11-8.04 (m, 1H), 4.14 (s, 3H).

### Step 5: 3-[2-(6,7-Difluoro-1-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a stirred mixture of 3-ethynyl-1-[(3S,5R)-5-(methoxymethyl)-1-(prop-2-enoyl)pyrrolidin-3-yl]-5-(methylamino)pyrazole-4-carboxamide (0.15 g, 0.45 mmol), 6,7-difluoro-5-iodo-1-methyl-1,3-benzodiazole (0.13 g, 0.45 mmol), CuI (17.24 mg, 0.09 mmol) and Pd(PPh₃)₂Cl₂ (31.77 mg, 0.04 mmol) in DMF (1.50 mL) was added TEA (0.19 mL, 1.86 mmol). The reaction mixture was degassed with argon for three times and stirred for 1 h at 70° C. The resulting mixture was concentrated under reduce pressure. The residue was purified by silica gel Column chromatography, eluted with 8% MeOH in DCM. The fractions contained desired product were combined and concentrated. The crude product was purified by Prep-HPLC with the following conditions: Column: XBridge Prep C18 OBD Column, 19×150 mm 5 μm; Mobile Phase A: Water (10 mmol/L NH₄HCO₃), Mobile Phase B: ACN; Flow rate: 20 mL/min; Gradient: 10 B to 50 B in 5.8 min, 254 nm; RT: 5.85 min. The fractions contained desired product were combined and concentrated to afford 3-[2-(6,7-difluoro-1-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (75.0 mg, 33%) as a white solid. MS ESI calculated for C₂₄H₂₅F₂N₇O₃[M+H]⁺, 498.20, found 498.15; ¹H NMR (300 MHz, DMSO-d₆) δ 8.33 (s, 1H), 7.80 (d, J=5.2 Hz, 1H), 7.45 (s, 1H), 6.81-6.49 (m, 3H), 6.16 (d, J=16.5 Hz, 1H), 5.82-5.53 (m, 1H), 5.25-5.20 (m, 1H), 4.46-4.32 (m, 1H), 3.99-3.95 (m, 4H), 3.92-3.75 (m, 1H), 3.47-3.42 (m, 2H), 3.31 (s, 3H), 2.95-2.91 (m, 3H), 2.46-2.43 (m, 1H), 2.31-2.29 (m, 1H).

### Example 102: 3-[2-(1,3-Benzothiazol-2-yl)ethynyl]-1-[(3S,5R)-5-(methoxymethyl)-1-(prop-2-enoyl)pyrrolidin-3-yl]-5-(methylamino)pyrazole-4-carboxamide

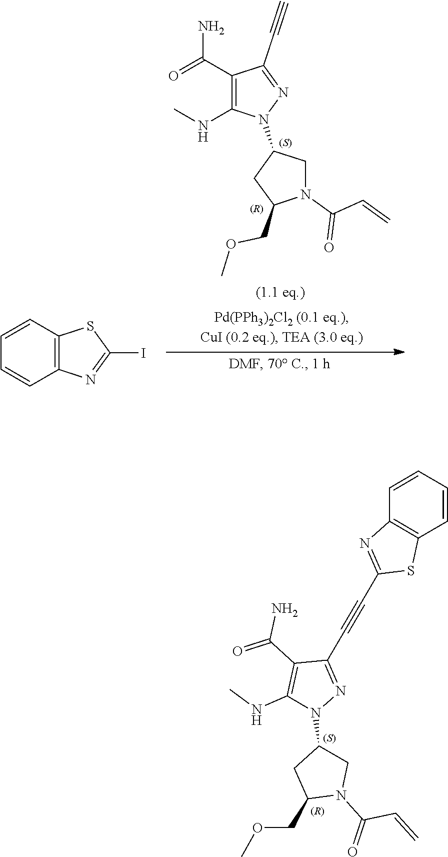

To a stirred mixture of 2-iodo-1,3-benzothiazole (0.3 g, 1.15 mmol), 3-ethynyl-1-[(3S,5R)-5-(methoxymethyl)-1-(prop-2-enoyl)pyrrolidin-3-yl]-5-(methylamino)pyrazole-4-carboxamide (0.42 g, 1.26 mmol), CuI (43.77 mg, 0.23 mmol) and Pd(PPh₃)₂Cl₂ (80.65 mg, 0.12 mmol) in DMF (6.00 mL) was added TEA (0.48 mL, 4.74 mmol). The reaction mixture was degassed with argon for three times and stirred for 1 h at 70° C. The resulting mixture was diluted with EtOAc (300 mL), washed with water (3×100 mL), dried over anhydrous Na₂SO₄ and filtered. The filtrate was concentrated under reduced pressure. The residue was purified by reverse flash chromatography with the following conditions: Column, C18 silica gel; mobile phase, ACN in water, 15% to 45% gradient in 40 min; detector, UV 254 nm. The fractions contained desired product were combined and concentrated to afford 3-[2-(1,3-benzothiazol-2-yl)ethynyl]-1-[(3S,5R)-5-(methoxymethyl)-1-(prop-2-enoyl)pyrrolidin-3-yl]-5-(methylamino)pyrazole-4-carboxamide (0.30 g, 56%) as a light yellow solid. MS ESI calculated for C₂₃H₂₄N₆O₃S [M+H]⁺, 465.16, found 465.25; ¹H NMR (400 MHz, CDCl₃) δ 8.15-8.08 (m, 1H), 7.95-7.88 (m, 1H), 7.62-7.46 (m, 2H), 6.91-6.82 (m, 1H), 6.76 (s, 1H), 6.60-6.38 (m, 2H), 5.82-5.73 (m, 1H), 5.61-5.21 (m, 2H), 4.64-4.40 (m, 1H), 4.16-3.98 (m, 2H), 3.95-3.89 (m, 1H), 3.55-3.44 (m, 1H), 3.39 (d, J=4.8 Hz, 3H), 3.06 (m, 3H), 2.77-2.63 (m, 1H), 2.46-2.30 (m, 1H).

### Example 103: 3-[2-(1,3-Benzothiazol-5-yl)ethynyl]-1-[(3S,5R)-5-(methoxymethyl)-1-(prop-2-enoyl)pyrrolidin-3-yl]-5-(methylamino)pyrazole-4-carboxamide

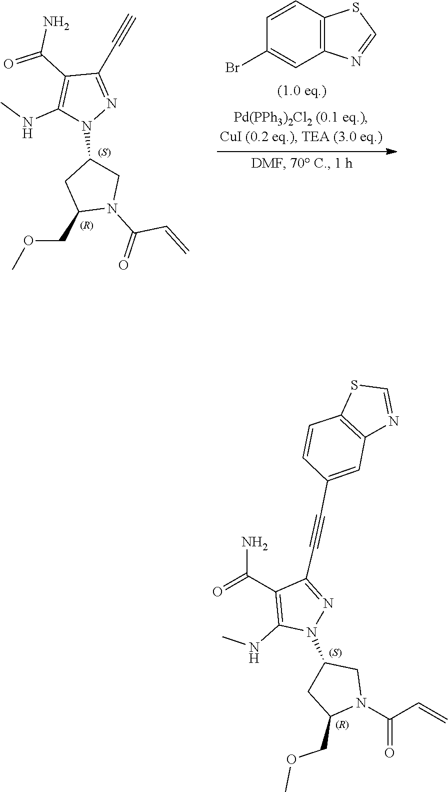

To a stirred mixture of 5-bromo-1,3-benzothiazole (64.60 mg, 0.30 mmol), 3-ethynyl-1-[(3S,5R)-5-(methoxymethyl)-1-(prop-2-enoyl) pyrrolidin-3-yl]-5-(methylamino) pyrazole-4-carboxamide (0.10 g, 0.30 mmol), Pd(PPh₃)₂Cl₂ (21.18 mg, 0.03 mmol) and CuI (11.49 mg, 0.06 mmol) in DMF (3.00 mL) was added TEA (91.61 mg, 0.91 mmol) at room temperature. The reaction mixture was degassed with argon for three times and stirred for 1 h at 70° C. under argon atmosphere. The resulting mixture was cooled down to room temperature and concentrated under reduced pressure. The residue was purified by silica gel Column chromatography, eluted with CH₂Cl₂/MeOH (10/1). The fractions contained desired product were combined and concentrated. The crude product was purified by Prep-HPLC with the following conditions: Column: XBridge Prep C18 OBD Column, 19×150 mm 5 μm; Mobile Phase A: Water (10 mmol/L NH₄HCO₃, Mobile Phase B: ACN; Flow rate: 20 mL/min; Gradient: 10 B to 50 B in 5.8 min; 210/254 nm; RT: 5.59 min. The fractions contained desired product were combined and concentrated to afford 3-[2-(1,3-benzothiazol-5-yl)ethynyl]-1-[(3S,5R)-5-(methoxymethyl)-1-(prop-2-enoyl)pyrrolidin-3-yl]-5-(methylamino)pyrazole-4-carboxamide (0.05 g, 32%) as an off-white solid. MS ESI calculated for C₂₃H₂₄N₆O₃S [M+H]⁺, 465.16, found 465.05; ¹H NMR (300 MHz, CDCl₃) δ 9.09 (s, 1H), 8.34 (s, 1H), 7.99 (d, J=8.3 Hz, 1H), 7.63 (d, J=8.4 Hz, 1H), 6.82 (s, 2H), 6.49-6.40 (m, 2H), 5.73 (dd, J=8.2, 4.4 Hz, 1H), 5.53 (s, 2H), 4.58-4.45 (m, 1H), 4.08-4.05 (m, 2H), 3.92-3.89 (m, 1H), 3.56-3.42 (m, 1H), 3.39-3.36 (m, 3H), 3.06-3.03 (m, 3H), 2.73-2.70 (m, 1H), 2.33-2.30 (m, 1H).

### Example 104: 3-(2-[Imidazo[1,2-a]pyridin-2-yl]ethynyl)-1-[(3S,5R)-5-(methoxymethyl)-1-(prop-2-enoyl)pyrrolidin-3-yl]-5-(methylamino)pyrazole-4-carboxamide

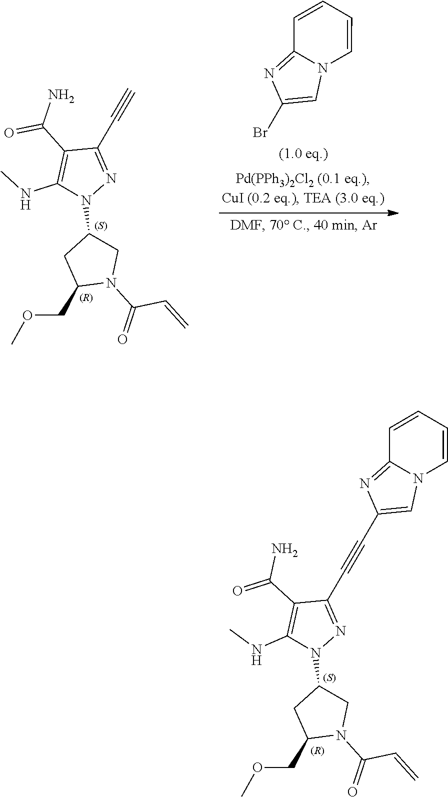

To a stirred mixture of 3-ethynyl-1-[(3S,5R)-5-(methoxymethyl)-1-(prop-2-enoyl)pyrrolidin-3-yl]-5-(methylamino)pyrazole-4-carboxamide (0.30 g, 0.90 mmol), 2-bromoimidazo[1,2-a]pyridine (0.18 g, 0.91 mmol), Pd(PPh₃)₂Cl₂ (63.54 mg, 0.09 mmol) and CuI (34.48 mg, 0.18 mmol) in DMF (4.00 mL) was added TEA (0.27 g, 2.72 mmol). The reaction mixture was degassed with argon for three times and stirred for 40 min at 70° C. The resulting mixture was concentrated under reduced pressure. The residue was purified by silica gel Column chromatography, eluted with MeOH in DCM (0-5%) to afford the crude product. The crude product was purified by Prep-HPLC with the following conditions: Column: SunFire Prep C18 OBD Column, 19×150 mm 5 μm 10 nm; Mobile Phase A: Water (0.1% FA), Mobile Phase B: ACN; Flow rate: 20 mL/min; Gradient: 5 B to 50 B in 5.8 min; 210/254 nm; RT: 5.58 min. The fractions contained desired product were combined and concentrated to afford 3-(2-[imidazo[1,2-a]pyridin-2-yl]ethynyl)-1-[(3S,5R)-5-(methoxymethyl)-1-(prop-2-enoyl)pyrrolidin-3-yl]-5-(methylamino)pyrazole-4-carboxamide (17.90 mg, 4%) as an orange solid. MS ESI calculated for C₂₃H₂₅N₇O₃ [M+H]⁺, 448.20, found 448.10; ¹H NMR (400 MHz, CDCl₃) δ 8.12 (d, J=6.8 Hz, 1H), 7.87 (s, 1H), 7.62 (d, J=9.2 Hz, 1H), 7.26 (d, J=8.3 Hz, 1H), 7.01 (s, 1H), 6.89 (d, J=6.8 Hz, 1H), 6.82-6.64 (m, 1H), 6.59-6.36 (m, 2H), 5.73-5.70 (m, 1H), 5.57-5.24 (m, 2H), 4.65-4.38 (m, 1H), 4.17-3.97 (m, 2H), 3.92-3.88 (m, 1H), 3.53-3.44 (m, 1H), 3.39 (d, J=4.1 Hz, 3H), 3.06-3.01 (m, 3H), 2.72-2.66 (m, 1H), 2.34-2.29 (m, 1H).

### Example 105: 1-((3S,5R)-1-Acryloyl-5-(methoxymethyl)pyrrolidin-3-yl)-3-((3-cyanoimidazo[1,2-a]pyridin-2-yl)ethynyl)-5-(methylamino)-1H-pyrazole-4-carboxamide

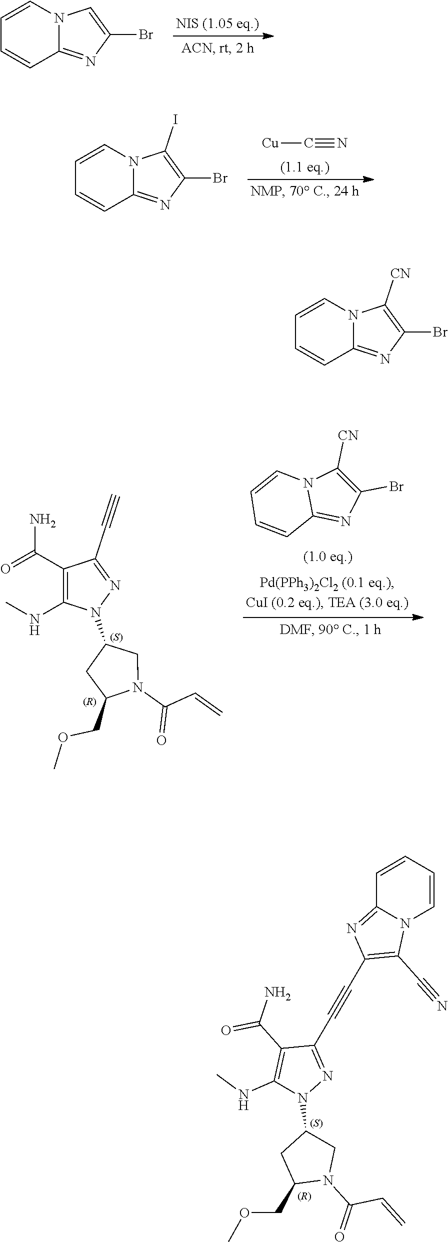

### Step 1: 2-Bromo-3-iodoimidazo[1,2-a]pyridine

To a stirred solution of 2-bromoimidazo[1,2-a]pyridine (1.60 g, 8.12 mmol) in ACN (16.00 mL) was added NIS (1.92 g, 8.53 mmol) in portions at room temperature under nitrogen atmosphere. The reaction mixture was stirred for 2 h at room temperature. The resulting mixture was concentrated under reduced pressure. The residue was purified by silica gel Column chromatography, eluted with CH₂Cl₂/MeOH (10/1), the fractions contained desired product were combined and concentrated to afford 2-bromo-3-iodoimidazo[1,2-a]pyridine (2.3 g, 87%) as a brown solid. MS ESI calculated for C₇H₄BrIN₂ [M+H]⁺, 322.86, 324.86, found 322.85, 324.85; ¹H NMR (400 MHz, CDCl₃) δ 8.10-8.07 (m, 1H), 7.61-7.52 (m, 1H), 7.31-7.23 (m, 1H), 6.98-6.96 (m, 1H).

### Step 2: 2-Bromoimidazo[1,2-a]pyridine-3-carbonitrile

To a stirred solution of 2-bromo-3-iodoimidazo[1,2-a]pyridine (1.00 g, 3.09 mmol) in NMP (10 mL) was added cuprous cyanide (0.31 g, 3.46 mmol) in portions at room temperature under nitrogen atmosphere. The reaction mixture was stirred for 24 h at 70° C. The resulting mixture was quenched by the addition of edta disodium salt dihydrate (120.00 mL, 0.32 mol) at room temperature and diluted with EtOAc (150 mL). The resulting mixture was stirred for additional 2 h at room temperature. The resulting mixture was extracted with EtOAc (3×100 mL). The combined organic layers were washed with brine (3×80 mL), dried over anhydrous Na₂SO₄ and filtered. The filtrate was concentrated under reduced pressure. The residue was purified by silica gel Column chromatography, eluted with CH₂Cl₂/MeOH (10/1). The fractions contained desired product were combined and concentrated to afford 2-bromoimidazo[1,2-a]pyridine-3-carbonitrile (0.14 g, 20%) as an off-white solid. MS ESI calculated for C₈H₄BrN₃ [M+H]⁺, 221.96, 223.96, found 222.00, 224.00; ¹H NMR (400 MHz, CDCl₃) δ 8.33-8.30 (m, 1H), 7.75-7.71 (m, 1H), 7.53-7.48 (m, 1H), 7.18-7.15 (m, 1H).

### Step 3: 1-((3S,5R)-1-Acryloyl-5-(methoxymethyl)pyrrolidin-3-yl)-3-((3-cyanoimidazo[1,2-a]pyridin-2-yl)ethynyl)-5-(methylamino)-1H-pyrazole-4-carboxamide

To a stirred mixture of 3-ethynyl-1-[(3S,5R)-5-(methoxymethyl)-1-(prop-2-enoyl)pyrrolidin-3-yl]-5-(methylamino)pyrazole-4-carboxamide (0.19 g, 0.57 mmol), 2-bromoimidazo[1,2-a]pyridine-3-carbonitrile (0.13 g, 0.57 mmol), CuI (0.02 g, 0.12 mmol) and Pd(PPh₃)₂Cl₂ (0.04 g, 0.05 mmol) in DMF (2.00 mL) was added TEA (0.24 mL, 1.72 mmol). The reaction mixture was degassed with argon for three times and stirred for 1 h at 90° C. The resulting mixture was diluted with water (20 mL), extracted with EA (3×20 mL). The combined organic layers were washed with brine (3×20 mL), dried over anhydrous Na₂SO₄ and filtered. The filtrate was concentrated under reduced pressure. The residue was purified by Prep-HPLC with the following conditions: Column: XBridge Prep C18 OBD Column, 19×150 mm 5 μm; Mobile Phase A: Water (10 mmol/L NH₄HCO₃), Mobile Phase B: ACN; Flow rate: 20 mL/min; Gradient: 25 B to 50 B in 6 min; 210/254 nm; RT: 5.58 min. The fractions contained desired product were combined and concentrated to afford 1-((3S,5R)-1-acryloyl-5-(methoxymethyl)pyrrolidin-3-yl)-3-((3-cyanoimidazo[1,2-a]pyridin-2-yl)ethynyl)-5-(methylamino)-1H-pyrazole-4-carboxamide (87.8 mg, 32%) as an off-white solid. MS ESI calculated for C₂₄H₂₄N₅O₃ [M+H]⁺, 473.20, found 473.15; ¹H NMR (400 MHz, DMSO-d₆) δ 8.68-8.65 (m, 1H), 7.86-7.82 (m, 1H), 7.69-7.64 (m, 1H), 7.49 (s, 1H), 7.33-7.29 (m, 1H), 6.97-6.40 (m, 3H), 6.18-7.15 (m, 1H), 5.69-5.66 (m, 1H), 5.28-5.23 (m, 1H), 4.48-4.44 (m, 1H), 4.13-3.67 (m, 2H), 3.66-3.41 (m, 2H), 3.31 (s, 3H), 2.94 (t, J=5.6 Hz, 3H), 2.64-2.61 (m, 1H), 2.34-2.25 (m, 1H).

### Example 106: 1-[(3S,5R)-5-(Methoxymethyl)-1-(prop-2-enoyl)pyrrolidin-3-yl]-5-(methylamino)-3-(2-[[1,2,4]triazolo[1,5-a]pyridin-6-yl]ethynyl)pyrazole-4-carboxamide

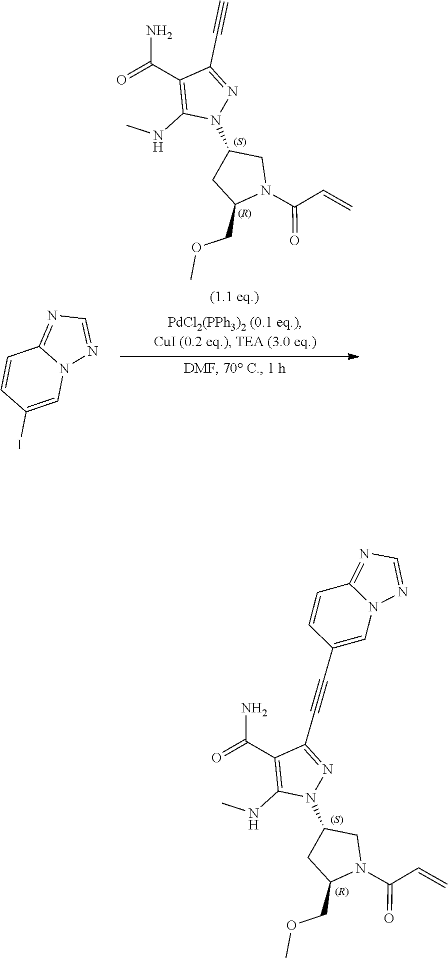

To a stirred mixture of 6-iodo-[1,2,4]triazolo[1,5-a]pyridine (0.15 g, 0.61 mmol), 3-ethynyl-1-[(3S,5R)-5-(methoxymethyl)-1-(prop-2-enoyl)pyrrolidin-3-yl]-5-(methylamino)pyrazole-4-carboxamide (0.22 g, 0.67 mmol), CuI (23.32 mg, 0.12 mmol) and Pd(PPh₃)₂Cl₂ (42.97 mg, 0.06 mmol) in DMF (2.00 mL) was added TEA (0.26 mL, 2.52 mmol). The reaction mixture was degassed with argon for three times and stirred for 1 h at 70° C. The resulting mixture was diluted with EtOAc (300 mL), washed with water (3×100 mL), dried over anhydrous Na₂SO₄ and filtered. The filtrate was concentrated under reduced pressure. The residue was purified by Prep-HPLC with the following conditions: Column: XBridge Prep C18 OBD Column, 19×150 mm 5 μm; Mobile Phase A: Water (10 mmol/L NH₄HCO₃), Mobile Phase B: ACN; Flow rate: 20 mL/min; Gradient: 15 B to 50 B in 6 min, 210/254 nm; RT: 5.8 min. The fractions contained desired product were combined and concentrated to afford 1-[(3S,5R)-5-(methoxymethyl)-1-(prop-2-enoyl)pyrrolidin-3-yl]-5-(methylamino)-3-(2-[[1,2,4]triazolo[1,5-a]pyridin-6-yl]ethynyl)pyrazole-4-carboxamide (0.11 g, 40%) as an off-white solid. MS ESI calculated for C₂₂H₂₄N₈O₃ [M+H]⁺, 449.20, found 449.30; ¹H NMR (400 MHz, CDCl₃) δ 8.86 (s, 1H), 8.44 (s, 1H), 7.85-7.78 (m, 1H), 7.65 (dd, J=9.2, 1.6 Hz, 1H), 6.64 (s, 1H), 6.59-6.37 (m, 2H), 5.80-5.69 (m, 1H), 5.61-5.23 (m, 2H), 4.66-4.35 (m, 1H), 4.14-3.95 (m, 2H), 3.95-3.89 (m, 1H), 3.55-3.42 (m, 1H), 3.39 (d, J=5.2 Hz, 3H), 3.06 (d, J=14.6 Hz, 3H), 2.76-2.64 (m, 1H), 2.46-2.29 (m, 1H).

### Example 107: 1-[(3S,5R)-5-(Methoxymethyl)-1-(prop-2-enoyl)pyrrolidin-3-yl]-5-(methylamino)-3-(2-[[1,2,4]triazolo[1,5-a]pyridin-7-yl]ethynyl)pyrazole-4-carboxamide

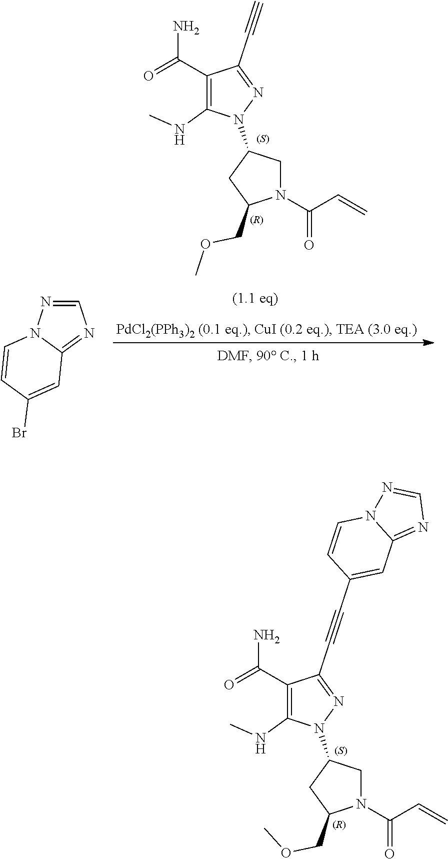

To a stirred mixture of 7-bromo-[1,2,4]triazolo[1,5-a]pyridine (0.15 g, 0.76 mmol), 3-ethynyl-1-[(3S,5R)-5-(methoxymethyl)-1-(prop-2-enoyl)pyrrolidin-3-yl]-5-(methylamino)pyrazole-4-carboxamide (0.27 g, 0.83 mmol), CuI (28.85 mg, 0.15 mmol) and Pd(PPh₃)₂Cl₂ (53.17 mg, 0.08 mmol) in DMF (2.00 mL) was added TEA (0.32 mL, 3.12 mmol). The reaction mixture was degassed with argon for three times and stirred for 1 h at 90° C. The resulting mixture was diluted with EtOAc (300 mL), washed with water (3×100 mL), dried over anhydrous Na₂SO₄ and filtered. The filtrate was concentrated under reduced pressure. The residue was purified by Prep-HPLC with the following conditions: Column: Atlantis HILIC OBD Column, 19×150 mm×5 μm; Mobile Phase A: Water (10 mmol/L NH₄HCO₃), Mobile Phase B: ACN; Flow rate: 20 mL/min; Gradient: 15 B to 50 B in 6 min; 210/254 nm; RT: 5.8 min. The fractions contained desired product were combined and concentrated to afford 1-[(3S,5R)-5-(methoxymethyl)-1-(prop-2-enoyl)pyrrolidin-3-yl]-5-(methylamino)-3-(2-[[1,2,4]triazolo[1,5-a]pyridin-7-yl]ethynyl)pyrazole-4-carboxamide (0.14 g, 40%) as an off-white solid. MS ESI calculated for C₂₂H₂₄N₅O₃ [M+H]⁺, 449.20, found 449.10; ¹H NMR (400 MHz, CDCl₃) δ 8.62 (d, J=7.2 Hz, 1H), 8.44 (s, 1H), 7.98 (s, 1H), 7.16 (dd, J=7.2, 1.2 Hz, 1H), 6.88-6.57 (m, 1H), 6.53-6.38 (m, 2H), 5.78-5.69 (m, 1H), 5.62-5.22 (m, 2H), 4.68-4.36 (m, 1H), 4.14-4.03 (m, 2H), 4.02-3.88 (m, 1H), 3.57-3.43 (m, 1H), 3.39 (d, J=5.2 Hz, 3H), 3.06 (d, J=14.4 Hz, 3H), 2.76-2.64 (m, 1H), 2.46-2.29 (m, 1H).

### Example 108: 3-[2-(1,3-Benzoxazol-5-yl)ethynyl]-1-[(3S,5R)-5-(methoxymethyl)-1-(prop-2-enoyl)pyrrolidin-3-yl]-5-(methylamino)pyrazole-4-carboxamide

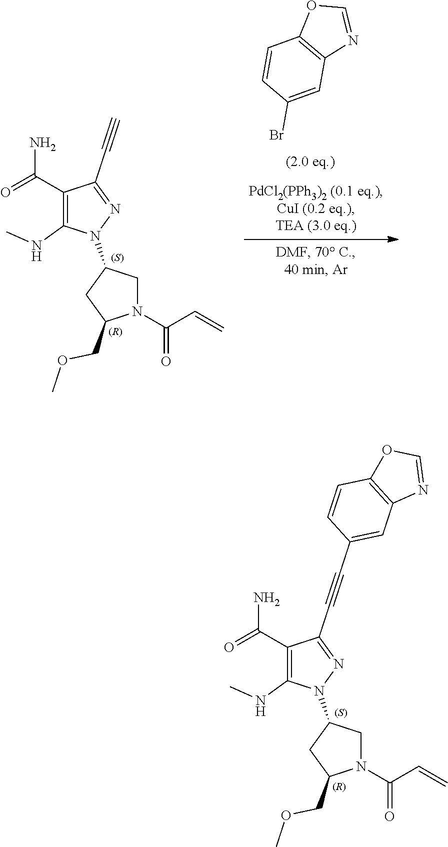

To a stirred solution of 3-ethynyl-1-[(3S,5R)-5-(methoxymethyl)-1-(prop-2-enoyl)pyrrolidin-3-yl]-5-(methylamino)pyrazole-4-carboxamide (0.12 g, 0.36 mmol), 5-bromo-1,3-benzoxazole (0.14 g, 0.72 mmol), Pd(PPh₃)₂Cl₂ (25.42 mg, 0.04 mmol) and CuI (13.79 mg, 0.07 mmol) in DMF (0.50 mL) was added TEA (0.11 g, 1.08 mmol) dropwise at room temperature. The reaction mixture was degassed with argon for three times and stirred for 40 minutes at 70° C. The resulting mixture was concentrated under reduced pressure. The residue was purified by silica gel Column chromatography, eluted with CH₂Cl₂/MeOH (10/1). The fractions contained desired product were combined and concentrated. The crude product was purified by Prep-HPLC with the following conditions: Column: XBridge Prep C18 OBD Column, 19×150 mm 5 μm; Mobile Phase A: Water (10 mmol/L NH₄HCO₃), Mobile Phase B: ACN; Flow rate: 20 mL/min; Gradient: 35 B to 55 B in 6 min; 210/254 nm. The fractions contained desired product were combined and concentrated to afford 3-[2-(1,3-benzoxazol-5-yl)ethynyl]-1-[(3S,5R)-5-(methoxymethyl)-1-(prop-2-enoyl)pyrrolidin-3-yl]-5-(methylamino)pyrazole-4-carboxamide (14.50 mg, 9%) as a white solid. MS ESI calculated for C₂₃H₂₄N₆O₄ [M+H]⁺, 449.20, found 449.15; ¹H NMR (300 MHz, CDCl₃) δ 8.16 (s, 1H), 7.99 (s, 1H), 7.60 (d, J=1.2 Hz, 2H), 6.78 (d, J=6.3 Hz, 2H), 6.57-6.26 (m, 2H), 5.70-5.51 (m, 1H), 5.49-4.45 (m, 2H), 4.56-4.45 (m, 1H), 4.15-3.96 (m, 2H), 3.90-3.67 (m, 1H), 3.59-3.28 (m, 4H), 3.04-2.87 (m, 3H), 2.78-2.59 (m, 1H), 2.34-2.12 (m, 1H).

### Example 109: 5-[(Cyclopropylmethyl)amino]-3-[2-(1-ethyl-4,6-difluoro-1,3-benzodiazol-5-yl)ethynyl]-1-[(3S,5R)-5-(methoxymethyl)-1-(prop-2-enoyl)pyrrolidin-3-yl]pyrazole-4-carboxamide

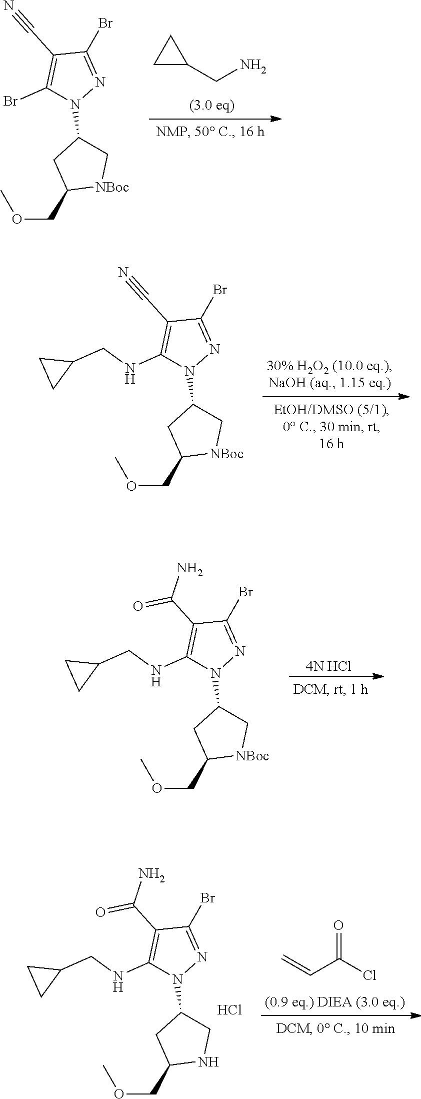

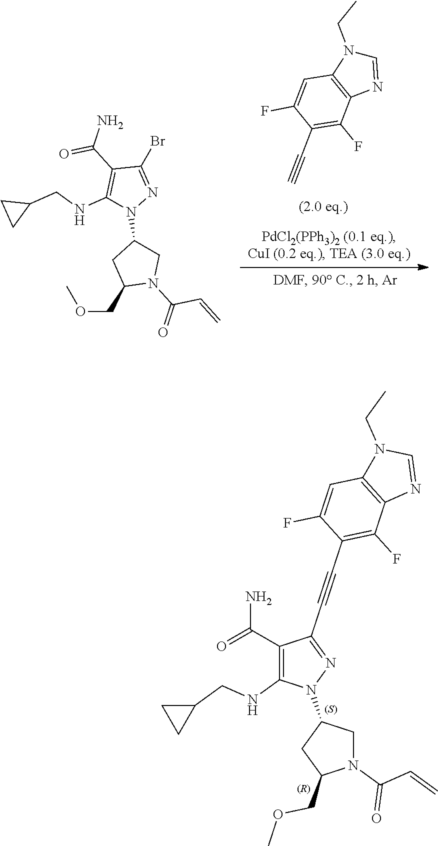

### Step 1: Tert-butyl (2R,4S)-4-[3-bromo-4-cyano-5-[(cyclopropylmethyl)amino]pyrazol-1-yl]-2-(methoxymethyl)pyrrolidine-1-carboxylate

To a stirred mixture of tert-butyl (2R,4S)-4-(3,5-dibromo-4-cyanopyrazol-1-yl)-2-(methoxymethyl)pyrrolidine-1-carboxylate (1.00 g, 2.15 mmol) in NMP (10.00 mL) was added 1-cyclopropylmethanamine (0.46 g, 6.46 mmol). The reaction mixture was stirred for 16 h at 50° C. The resulting mixture was poured into water (50 mL) and extracted with EtOAc (3×50 mL). The combined organic layers were washed with water (4×30 mL), dried over anhydrous Na₂SO₄ and filtrated. The filtrate was concentrated under reduced pressure to afford tert-butyl (2R,4S)-4-[3-bromo-4-cyano-5-[(cyclopropylmethyl)amino]pyrazol-1-yl]-2-(methoxymethyl)pyrrolidine-1-carboxylate (1.0 g, 99%) as a yellow oil which was used in the next step directly without further purification. MS ESI calculated for C₁₉H₂₈BrN₅O₃ [M+H−56]⁺, 398.14, 400.14. found 398.20, 400.20; ¹H NMR (400 MHz, CDCl₃) δ 6.40 (s, 1H), 4.99-4.93 (m, 1H), 4.72 (s, 1H), 4.26-4.14 (m, 2H), 3.78-3.56 (m, 4H), 3.47-3.30 (m, 2H), 2.59 (s, 1H), 2.25 (s, 1H), 2.06 (s, 1H), 1.28 (m, 9H), 1.20-1.09 (m, 1H), 0.72-0.58 (m, 2H), 0.32 (m, 2H).

### Step 2: Tert-butyl (2R,4S)-4-[3-bromo-4-carbamoyl-5-[(cyclopropylmethyl)amino]pyrazol-1-yl]-2-(methoxymethyl)pyrrolidine-1-carboxylate

To a stirred solution of tert-butyl (2R,4S)-4-[3-bromo-4-cyano-5-[(cyclopropylmethyl)amino]pyrazol-1-yl]-2-(methoxymethyl)pyrrolidine-1-carboxylate (1.00 g, 2.20 mmol) in EtOH (10.00 mL) and DMSO (2.00 mL) were added NaOH (0.5 M in water) (0.10 mL, 2.53 mmol) and H₂O₂ (1.71 mL, 22.01 mmol, 30%) dropwise at 0° C. The reaction mixture was stirred for 30 min at 0° C. and 16 h at room temperature. The resulting mixture was diluted with water (20 mL) and extracted with EA (3×20 mL). The combined organic layers were washed with water (2×20 mL), dried over anhydrous Na₂SO₄ and filtered. The filtrate was concentrated under reduced pressure. The residue was purified by silica gel Column chromatography, eluted with EA in PE (20-70%). The fractions contained desired product were combined and concentrated to afford tert-butyl (2R,4S)-4-[3-bromo-4-carbamoyl-5-[(cyclopropylmethyl)amino]pyrazol-1-yl]-2-(methoxymethyl)pyrrolidine-1-carboxylate (0.80 g, 77%) as a yellow solid. MS ESI calculated for C₁₉H₃₀BrN₅O₄ [M+H]⁺, 472.15, 474.15, found 472.10, 474.10; ¹H NMR (400 MHz, CDCl₃) δ 6.69 (d, J=100.4 Hz, 2H), 6.06-5.29 (m, 1H), 5.13 (brs, 1H), 4.24-4.04 (m, 1H), 3.79-3.62 (m, 3H), 3.48-3.28 (m, 3H), 3.05-3.02 (m, 2H), 2.59-2.56 (m, 1H), 2.32-1.98 (m, 1H), 1.59-1.38 (m, 9H), 1.37-1.01 (m, 2H), 0.58-0.52 (m, 2H), 0.29-0.25 (m, 2H).

### Step 3: 3-Bromo-5-[(cyclopropylmethyl)amino]-1-[(3S,5R)-5-(methoxymethyl)pyrrolidin-3-yl]pyrazole-4-carboxamide hydrochloride

To a stirred solution of tert-butyl (2R,4S)-4-[3-bromo-4-carbamoyl-5-[(cyclopropylmethyl)amino]pyrazol-1-yl]-2-(methoxymethyl)pyrrolidine-1-carboxylate (0.80 g, 1.69 mmol) in DCM (8.00 mL) was added HCl (4 M in EA) (4.00 mL) dropwise at 0° C. The reaction mixture was stirred for 1 h at room temperature. The resulting mixture was concentrated under reduced pressure to afford 3-bromo-5-[(cyclopropylmethyl)amino]-1-[(3S,5R)-5-(methoxymethyl)pyrrolidin-3-yl]pyrazole-4-carboxamide hydrochloride (0.70 g, 99%) as a yellow solid which was used in the next step directly without further purification. MS ESI calculated for C₁₄H₂₂BrN₅O₂ [M+H]⁺, 372.10, 374.10, found 372.15, 374.15; ¹H NMR (400 MHz, CD₃OD) δ 5.51-5.32 (m, 1H), 4.36-4.25 (m, 2H), 3.81-3.68 (m, 3H), 3.67-3.55 (m, 1H), 3.47 (s, 3H), 3.18-3.05 (m, 2H), 2.41-2.39 (m, 2H), 2.04 (s, 2H), 1.20-1.18 (m, 1H), 1.16-1.02 (m, 1H), 0.63-0.52 (m, 2H), 0.32-0.24 (m, 2H).

### Step 4: 1-[(2R,4S)-4-[4-(1-Aminoethenyl)-3-bromo-5-[(cyclopropylmethyl)amino]pyrazol-1-yl]-2-(methoxymethyl)pyrrolidin-1-yl]prop-2-en-1-one

To a stirred solution of 3-bromo-5-[(cyclopropylmethyl)amino]-1-[(3S,5R)-5-(methoxymethyl)pyrrolidin-3-yl]pyrazole-4-carboxamide hydrochloride (0.35 g, 0.86 mmol) and DIEA (0.33 g, 2.57 mmol) in DCM (4.00 mL) was added acryloyl chloride (69.75 mg, 0.77 mmol) dropwise at 0° C. under N₂ atmosphere. The reaction mixture was stirred for 10 min at 0° C. The resulting mixture was quenched with water (8 mL) and extracted with EA (3×10 mL). The combined organic layers were washed with water (20 mL), dried over anhydrous Na₂SO₄ and filtered. The filtrate was concentrated under reduced pressure. The residue was purified by silica gel Column chromatography, eluted with MeOH in DCM (0-10%). The fractions contained desired product were combined and concentrated to afford 1-[(2R,4S)-4-[4-(1-aminoethenyl)-3-bromo-5-[(cyclopropylmethyl)amino]pyrazol-1-yl]-2-(methoxymethyl)pyrrolidin-1-yl]prop-2-en-1-one (0.20 g, 55%) as a white solid. MS ESI calculated for C₁₇H₂₄BrN₅O₃ [M+H]⁺, 426.11, 428.11, found 426.20, 428.20; ¹H NMR (400 MHz, CDCl₃) δ 6.57 (s, 1H), 6.42-6.41 (m, 2H), 5.73-5.71 (m, 1H), 5.43-5.30 (m, 1H), 4.54-4.50 (m, 1H), 3.98 (d, J=8.2 Hz, 2H), 3.95-3.80 (m, 1H), 3.52-3.40 (m, 1H), 3.18-3.08 (m, 4H), 3.12-2.98 (m, 2H), 2.62-2.20 (m, 2H), 1.27-1.12 (m, 2H), 0.68-0.54 (m, 2H), 0.36-0.24 (m, 2H).

### Step 5: 5-[(Cyclopropylmethyl)amino]-3-[2-(1-ethyl-4,6-difluoro-1,3-benzodiazol-5-yl)ethynyl]-1-[(3S,5R)-5-(methoxymethyl)-1-(prop-2-enoyl)pyrrolidin-3-yl]pyrazole-4-carboxamide

To a stirred solution of 3-bromo-5-[(cyclopropylmethyl)amino]-1-[(3S,5R)-5-(methoxymethyl)-1-(prop-2-enoyl)pyrrolidin-3-yl]pyrazole-4-carboxamide (0.15 g, 0.35 mmol), 1-ethyl-5-ethynyl-4,6-difluoro-1,3-benzodiazole (0.15 g, 0.70 mmol), CuI (13.40 mg, 0.07 mmol) and Pd(PPh₃)₂Cl₂ (24.70 mg, 0.04 mmol) in DMF (4.00 mL) was added TEA (0.11 g, 1.06 mmol). The reaction mixture was degassed with argon for three times and stirred for 2 h at 90° C. The resulting mixture was cooled down and concentrated under reduced pressure. The residue was purified by silica gel Column chromatography, eluted with MeOH in DCM (0-5%) to afford the crude product. Then the crude product was purified by Prep-HPLC with the following conditions: Column: X-Bridge Prep C18 OBD Column, 19×150 mm 5 μm; Mobile Phase A: Water (10 mmol/L NH₄HCO₃), Mobile Phase B: ACN; Flow rate: 20 mL/min; Gradient: 10 B to 50 B in 5.8 min, 210/254 nm; RT: 5.58 min. The fractions contained desired product were combined and concentrated to afford 5-[(cyclopropylmethyl)amino]-3-[2-(1-ethyl-4,6-difluoro-1,3-benzodiazol-5-yl)ethynyl]-1-[(3S,5R)-5-(methoxymethyl)-1-(prop-2-enoyl)pyrrolidin-3-yl]pyrazole-4-carboxamide (43.4 mg, 22%) as a white solid. MS ESI calculated for C₂₈H₃₁F₂N₇O₃[M+H]⁺, 552.25, found 552.20; ¹H NMR (400 MHz, CDCl₃) δ 8.05 (s, 1H), 7.04 (d, J=8.2 Hz, 2H), 6.66-6.34 (m, 2H), 5.72-5.69 (m, 1H), 5.54-5.17 (m, 2H), 4.64-4.36 (m, 1H), 4.25-4.24 (m, 2H), 4.09-4.06 (m, 2H), 3.92-3.91 (m, 1H), 3.58-3.44 (m, 1H), 3.39 (d, J=5.0 Hz, 3H), 3.22-2.90 (m, 2H), 2.84-2.63 (m, 1H), 2.41-2.30 (m, 1H), 1.71-1.59 (m, 4H), 1.25-1.06 (m, 1H), 0.65-0.61 (m, 2H), 0.33-0.29 (m, 2H).

### Example 110: 3-[2-[1-(Difluoromethyl)-4,6-difluoro-2-methyl-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide

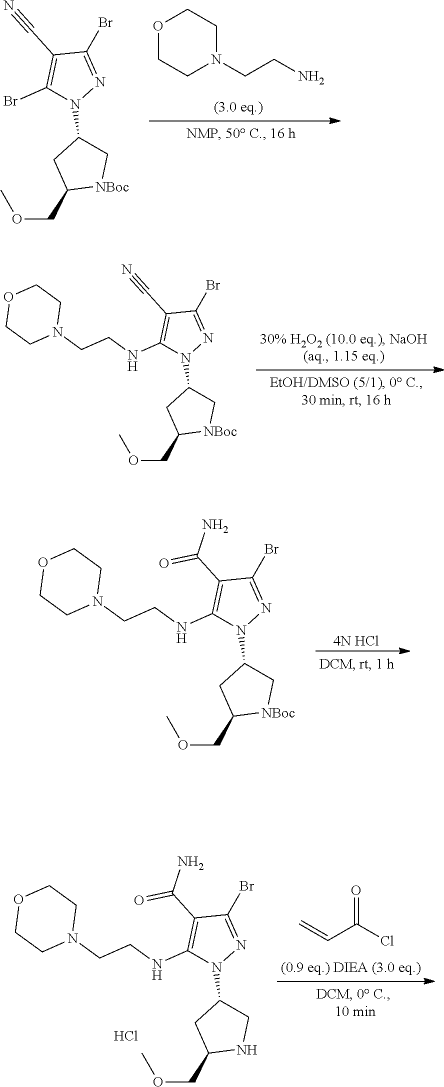

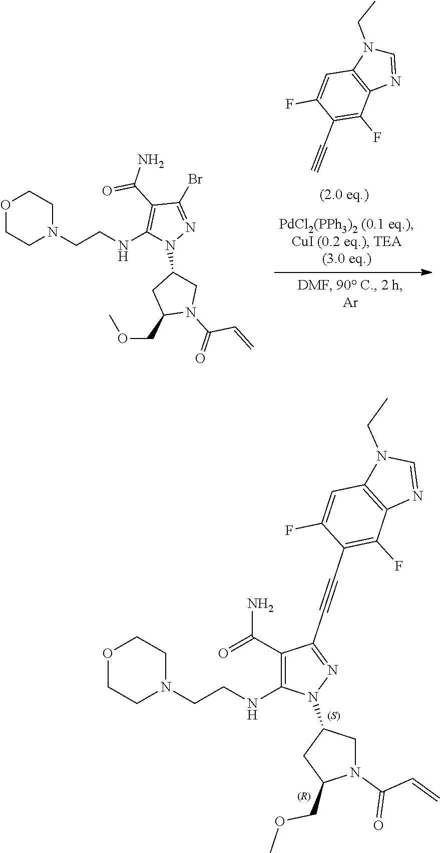

### Step 1: Tert-butyl (2R,4S)-4-(3-bromo-4-cyano-5-[[2-(morpholin-4-yl)ethyl]amino]pyrazol-1-yl)-2-(methoxymethyl)pyrrolidine-1-carboxylate

To a stirred mixture of tert-butyl (2R,4S)-4-(3,5-dibromo-4-cyanopyrazol-1-yl)-2-(methoxymethyl)pyrrolidine-1-carboxylate (1.00 g, 2.15 mmol) in NMP (10.00 mL) was added N-aminoethylmorpholine (0.84 g, 6.46 mmol). The reaction mixture was stirred for 16 h at 50° C. The resulting mixture was poured into water (100 mL) and extracted with EA (3×150 mL). The combined organic layers were washed with brine (2×200 mL), dried over anhydrous Na₂SO₄ and filtrated. The filtrate was concentrated under reduced pressure to afford tert-butyl (2R,4S)-4-(3-bromo-4-cyano-5-[[2-(morpholin-4-yl)ethyl]amino]pyrazol-1-yl)-2-(methoxymethyl)pyrrolidine-1-carboxylate (1.1 g, 99%) as a yellow oil which was used in the next step directly without further purification. MS ESI calculated for C₂₁H₃₃BrN₆O₄[M+H]⁺, 513.17, 515.17, found 513.25, 515.25; ¹H NMR (400 MHz, CDCl₃) δ 6.37 (s, 1H), 5.32 (s, 1H), 5.02-5.00 (m, 1H), 4.73 (s, 1H), 4.16 (d, J=7.2 Hz, 1H), 3.83-3.56 (m, 7H), 3.52-3.40 (m, 2H), 2.89-2.83 (m, 3H), 2.79-2.50 (m, 2H), 2.40-2.38 (m, 3H), 2.33-2.19 (m, 1H), 2.10-1.99 (m, 1H), 1.32-1.29 (m, 9H).

### Step 2: Tert-butyl (2R,4S)-4-(3-bromo-4-formyl-5-[[2-(morpholin-4-yl)ethyl]amino]pyrazol-1-yl)-2-(methoxymethyl)pyrrolidine-1-carboxylate

To a stirred solution of tert-butyl (2R,4S)-4-(3-bromo-4-methyl-5-[[2-(morpholin-4-yl)ethyl]amino]pyrazol-1-yl)-2-(methoxymethyl)pyrrolidine-1-carboxylate (0.95 g, 1.89 mmol) in EtOH (10.00 mL) and DMSO (2.00 mL) were added NaOH (0.5 M in water) (86.97 mg, 2.17 mmol) and H₂O₂ (1.47 mL, 43.17 mmol, 30%) dropwise at 0° C. The reaction mixture was stirred for 30 min at 0° C. and 16 h at room temperature. The resulting mixture was diluted with water (200 mL) and extracted with EA (3×150 mL). The combined organic layers were washed with water (2×200 mL), dried over anhydrous Na₂SO₄ and filtered. The filtrate was concentrated under reduced pressure. The residue was purified by silica gel Column chromatography, eluted with EA in PE (50-100%). The fractions contained desired product were combined and concentrated to afford tert-butyl (2R,4S)-4-(3-bromo-4-formyl-5-[[2-(morpholin-4-yl)ethyl]amino]pyrazol-1-yl)-2-(methoxymethyl)pyrrolidine-1-carboxylate (0.65 g, 66%) as a white solid. MS ESI calculated for C₂₁H₃₅BrN₆O₅ [M+H]⁺, 531.19, 533.19, found 531.20, 533.20; ¹H NMR (400 MHz, CDCl₃) δ 7.09-6.96 (m, 1H), 6.59 (s, 1H), 5.48 (s, 1H), 5.25-5.03 (m, 1H), 4.16 (s, 1H), 3.78-2.75 (m, 5H), 3.56 (s, 1H), 3.50 (s, 2H), 3.45-3.37 (m, 1H), 3.38-3.34 (m, 3H), 3.31 (d, J=8.1 Hz, 2H), 2.64-2.60 (m, 2H), 2.70-2.50 (m, 3H), 2.24-2.21 (m, 1H), 1.79-1.75 (m, 1H), 1.46-1.48 (m, 9H).

### Step 3: 3-Bromo-1-[(3S,5R)-5-(methoxymethyl)pyrrolidin-3-yl]-5-[[2-(morpholin-4-yl)ethyl]amino]pyrazole-4-carboxamide hydrochloride

To a stirred mixture of tert-butyl (2R,4S)-4-(3-bromo-4-carbamoyl-5-[[2-(morpholin-4-yl)ethyl]amino]pyrazol-1-yl)-2-(methoxymethyl)pyrrolidine-1-carboxylate (0.65 g, 1.22 mmol) in DCM (6.00 mL) was added HCl (4 M in EA) (3.00 mL) dropwise at 0° C. The reaction mixture was stirred for 1 h at room temperature. The resulting mixture was concentrated under reduced pressure to afford 3-bromo-1-[(3S,5R)-5-(methoxymethyl)pyrrolidin-3-yl]-5-[[2-(morpholin-4-yl)ethyl]amino]pyrazole-4-carboxamide hydrochloride (0.60 g, 99%) as a yellow solid which was used in the next step directly without further purification. MS ESI calculated for C₁₆H₂₇BrN₆O₃ [M+H]⁺, 431.13, 433.13, found 431.15, 433.15; ¹H NMR (400 MHz, CD₃OD) δ 5.51 (s, 1H), 5.42 (s, 1H), 4.35-4.32 m, 1H), 4.08 (d, J=13.0 Hz, 2H), 4.00-3.90 (m, 2H), 3.84-3.81 (m, 1H), 3.77-3.71 (m, 2H), 3.69-3.58 (m, 4H), 3.61-3.53 (m, 2H), 3.47 (s, 3H), 3.41-3.34 (m, 2H), 3.26-3.22 (m, 2H), 2.52-2.34 (m, 2H), 2.04-2.02 (m, 1H), 1.34-1.16 (m, 1H).

### Step 4: 3-[2-[1-(Difluoromethyl)-4,6-difluoro-2-methyl-1,3-benzodiazol-5-yl]ethynyl]-1-[(3S,5R)-5-(methoxymethyl)-1-(prop-2-enoyl)pyrrolidin-3-yl]-5-(methylamino)pyrazole-4-carboxamide

To a stirred mixture of 3-bromo-1-[(3S,5R)-5-(methoxymethyl)pyrrolidin-3-yl]-5-[[2-(morpholin-4-yl)ethyl]amino]pyrazole-4-carboxamide hydrochloride (0.60 g, 1.28 mmol) and DIEA (0.67 mL, 5.18 mmol) in DCM (6.00 mL) was added acryloyl chloride (0.11 g, 1.15 mmol) dropwise at 0° C. under N₂ atmosphere. The reaction mixture was stirred for 10 min at 0° C. The resulting mixture was quenched with water (8 mL) and extracted with EA (3×20 mL). The combined organic layers were washed with brine (20 mL), dried over anhydrous Na₂SO₄ and filtered. The filtrate was concentrated under reduced pressure. The residue was purified by silica gel Column chromatography, eluted with MeOH in DCM (0-5%). The fractions contained desired product were combined and concentrated to afford 3-bromo-1-[(3S,5R)-5-(methoxymethyl)-1-(prop-2-enoyl)pyrrolidin-3-yl]-5-[[2-(morpholin-4-yl)ethyl]amino]pyrazole-4-carboxamide (0.37 g, 59%) as a yellow solid. MS ESI calculated for C₁₉H₂₉BrN₆O₄ [M+H]⁺, 485.14, 487.14, found 485.20, 487.20; ¹H NMR (300 MHz, CDCl₃) δ 7.10 (s, 1H), 6.60-6.38 (m, 2H), 5.75-5.72 (m, 1H), 5.39-5.35 (m, 1H), 4.55 (d, J=9.2 Hz, 1H), 3.99 (d, J=8.3 Hz, 2H), 3.95-3.81 (m, 1H), 3.78-3.74 (m, 4H), 3.49-3.45 (m, 1H), 3.46-3.27 (m, 5H), 2.66-2.64 (m, 2H), 2.70-2.54 (m, 1H), 2.54-2.51 (m, 4H), 2.32-2.19 (m, 1H), 1.30-1.28 (m, 1H).

### Step 5: 3-[2-(1-Ethyl-4,6-difluoro-1,3-benzodiazol-5-yl)ethynyl]-1-[(3S,5R)-5-(methoxymethyl)-1-(prop-2-enoyl)pyrrolidin-3-yl]-5-[[2-(morpholin-4-yl)ethyl]amino]pyrazole-4-carboxamide

To a stirred solution of 3-bromo-1-[(3S,5R)-5-(methoxymethyl)-1-(prop-2-enoyl)pyrrolidin-3-yl]-5-[[2-(morpholin-4-yl)ethyl]amino]pyrazole-4-carboxamide (0.19 g, 0.39 mmol), 1-ethyl-5-ethynyl-4,6-difluoro-1,3-benzodiazole (0.16 g, 0.78 mmol), CuI (14.91 mg, 0.08 mmol) and Pd(PPh₃)₂Cl₂ (27.48 mg, 0.04 mmol) in DMF (4.00 mL) was added TEA (0.12 g, 1.17 mmol) dropwise at room temperature. The reaction mixture was degassed with argon for three times and stirred for 2 h at 90° C. The resulting mixture was cooled down and concentrated under reduced pressure. The residue was purified by silica gel Column chromatography, eluted with MOH in DCM (0-5%) to afford the crude product. Then the crude product was purified by Prep-HPLC with the following conditions: Column: X-Bridge Prep C18 OBD Column, 19×150 mm 5 μm; Mobile Phase A: Water (10 mmoL/L NH₄HCO₃), Mobile Phase B: ACN; Flow rate: 20 mL/min; Gradient: 25 B to 50 B in 6 min, 210/254 nm; RT: 5.58 min. The fractions contained desired product were combined and concentrated to afford 3-[2-(1-ethyl-4,6-difluoro-1,3-benzodiazol-5-yl)ethynyl]-1-[(3S,5R)-5-(methoxymethyl)-1-(prop-2-enoyl)pyrrolidin-3-yl]-5-[[2-(morpholin-4-yl)ethyl]amino]pyrazole-4-carboxamide (84.6 mg, 35%) as a white solid. MS ESI calculated for C₃₀H₃₆F₂N₈O₄[M+H]+, 611.28, found 611.25; ¹H NMR (300 MHz, CDCl₃) δ 7.94 (s, 1H), 7.15-6.96 (m, 2H), 6.81 (d, J=45.9 Hz, 1H), 6.57-6.37 (m, 2H), 5.71-5.68 (m, 1H), 5.50-5.19 (m, 2H), 4.64-4.38 (m, 1H), 4.20-4.16 (m, 2H), 4.15-3.84 (m, 3H), 3.79-3.74 (m, 4H), 3.50-3.47 (m, 1H), 3.37-3.32 (m, 5H), 2.79-2.58 (m, 3H), 2.55-2.51 (m, 4H), 2.43-2.21 (m, 1H), 1.60-1.54 (m, 3H).

### Example 111: 3-[2-(1-Ethyl-4,6-difluoro-1,3-benzodiazol-5-yl)ethynyl]-1-[(3S,5R)-5-(methoxymethyl)-1-(prop-2-enoyl)pyrrolidin-3-yl]-5-[[3-(morpholin-4-yl)propyl]amino]pyrazole-4-carboxamide

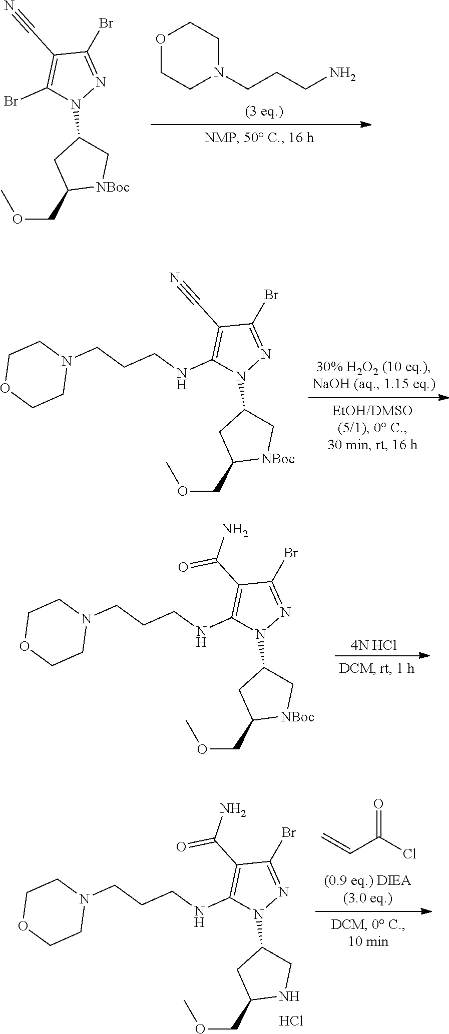

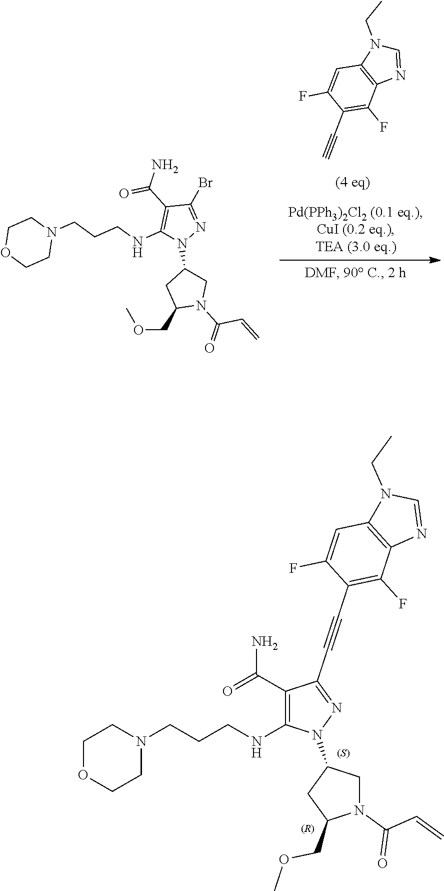

### Step 1: Tert-butyl (2R,4S)-4-(3-bromo-4-cyano-5-[[3-(morpholin-4-yl)propyl]amino]pyrazol-1-yl)-2-(methoxymethyl)pyrrolidine-1-carboxylate

To a stirred mixture of tert-butyl (2R,4S)-4-(3,5-dibromo-4-cyanopyrazol-1-yl)-2-(methoxymethyl)pyrrolidine-1-carboxylate (1.00 g, 2.15 mmol) in NMP (10.00 mL) was added 4-morpholinepropanamine (0.93 g, 6.46 mmol) dropwise at room temperature. The reaction mixture was stirred for 16 h at 50° C. The resulting mixture was poured into water (100 mL) and extracted with EA (3×50 mL). The combined organic layers were washed with brine (2×100 mL), dried over anhydrous Na₂SO₄, filtrated and concentrated under reduced pressure to afford tert-butyl (2R,4S)-4-(3-bromo-4-cyano-5-[[3-(morpholin-4-yl)propyl]amino]pyrazol-1-yl)-2-(methoxymethyl)pyrrolidine-1-carboxylate (1.0 g, 88%) as a yellow oil which was used in the next step directly without further purification. MS ESI calculated for C₂₂H₃₅BrN₆O₄ [M+H]⁺, 526.19, found 527.25; ¹H NMR (400 MHz, CDCl₃) δ 6.50 (s, 1H), 3.75-3.70 (m, 5H), 3.63-3.58 (m, 5H), 3.38-3.34 (m, 5H), 2.40-2.35 (m, 8H), 2.00-1.75 (m, 2H), 1.46 (s, 9H).

### Step 2: Tert-butyl (2R,4S)-4-(3-bromo-4-carbamoyl-5-[[3-(morpholin-4-yl)propyl]amino]pyrazol-1-yl)-2-(methoxymethyl)pyrrolidine-1-carboxylate

To a stirred solution of tert-butyl (2R,4S)-4-(3-bromo-4-cyano-5-[[3-(morpholin-4-yl)propyl]amino]pyrazol-1-yl)-2-(methoxymethyl)pyrrolidine-1-carboxylate (1.00 g, 1.90 mmol) in EtOH (10.00 mL) and DMSO (2.00 mL) were added NaOH (0.5 M in water) (4.36 mL, 2.18 mmol) and H₂O₂ (0.64 g, 18.96 mmol, 30%) dropwise at 0° C. The reaction mixture was stirred for 30 min at 0° C. and 16 h at room temperature. The resulting mixture was diluted with water (100 mL) and extracted with EA (3×100 mL). The combined organic layers were washed with brine (100 mL), dried over anhydrous Na₂SO₄ and filtered. The filtrate was concentrated under reduced pressure. The residue was purified by silica gel Column chromatography, eluted with MeOH in DCM (0-5%). The fractions contained desired product were combined and concentrated to afford tert-butyl (2R,4S)-4-(3-bromo-4-carbamoyl-5-[[3-(morpholin-4-yl)propyl]amino]pyrazol-1-yl)-2-(methoxymethyl)pyrrolidine-1-carboxylate (1 g, 97%) as a yellow solid. MS ESI calculated for C₂₂H₃₇BrN₆O₅ [M+H]⁺, 545.20, 547.20, found 545.25, 547.25; ¹H NMR (400 MHz, CDCl₃) δ 6.74 (s, 1H), 6.59 (s, 1H), 6.44 (s, 1H), 4.18 (d, J=18.4 Hz, 1H), 3.73-3.69 (m, 5H), 3.49 (s, 1H), 3.42-3.34 (m, 1H), 3.37 (s, 3H), 3.29-3.25 (m, 2H), 2.86-2.82 (m, 2H), 2.47-2.43 (m, 5H), 2.39-2.31 (m, 1H), 2.25-2.18 (m, 1H), 2.08-1.96 (m, 1H), 1.84-1.76 (m, 2H), 1.47 (s, 9H).

### Step 3: 3-Bromo-1-[(3S,5R)-5-(methoxymethyl)pyrrolidin-3-yl]-5-[[3-(morpholin-4-yl)propyl]amino]pyrazole-4-carboxamide hydrochloride

To a stirred solution of tert-butyl (2R,4S)-4-(3-bromo-4-carbamoyl-5-[[3-(morpholin-4-yl)propyl]amino]pyrazol-1-yl)-2-(methoxymethyl)pyrrolidine-1-carboxylate (1.00 g, 1.83 mmol) in DCM (10.00 mL) was added HCl (4 M in EA) (5.00 mL) dropwise at 0° C. The reaction mixture was stirred for 1 h at room temperature. The resulting mixture was concentrated under reduced pressure to afford 3-bromo-1-[(3S,5R)-5-(methoxymethyl)pyrrolidin-3-yl]-5-[[3-(morpholin-4-yl)propyl]amino]pyrazole-4-carboxamide hydrochloride (0.8 g, 91%) as a yellow solid which was used in the next step directly without further purification. MS ESI calculated for C₁₇H₂₉BrN₆O₃ [M+H]⁺, 445.15, 447.15, found 445.20, 447.20; ¹H NMR (300 MHz, CDCl₃) δ 6.37 (s, 1H), 5.57 (s, 3H), 5.30 (s, 1H), 4.99-4.95 (m, 1H), 4.12-4.09 (m, 1H), 3.45-3.42 (m, 2H), 2.90-2.85 (m, 2H), 2.56-2.51 (m, 1H), 2.08-2.05 (m, 1H), 2.07-2.02 (m, 5H), 1.27-1.21 (m, 10H).

### Step 4: 3-Bromo-1-[(3S,5R)-5-(methoxymethyl)-1-(prop-2-enoyl)pyrrolidin-3-yl]-5-[[3-(morpholin-4-yl)propyl]amino]pyrazole-4-carboxamide

To a stirred mixture of 3-bromo-1-[(3S,5R)-5-(methoxymethyl)pyrrolidin-3-yl]-5-[[3-(morpholin-4-yl)propyl]amino]pyrazole-4-carboxamide hydrochloride (0.80 g, 1.66 mmol) and DIEA (0.64 g, 4.98 mmol) in DCM (10.00 mL) was added acryloyl chloride (5.97 mL, 1.45 mmol, 0.25 M in DCM) dropwise at 0° C. under N₂ atmosphere. The reaction mixture was stirred for 10 min at 0° C. The resulting mixture was diluted with water (50 mL) and extracted with EA (3×50 mL). The combined organic layers were washed with water (100 mL), dried over anhydrous Na₂SO₄ and filtered. The filtrate was concentrated under reduced pressure. The residue was purified by silica gel Column chromatography, eluted with MeOH in DCM (0-10%). The fractions contained desired product were combined and concentrated to afford 3-bromo-1-[(3S,5R)-5-(methoxymethyl)-1-(prop-2-enoyl)pyrrolidin-3-yl]-5-[[3-(morpholin-4-yl)propyl]amino]pyrazole-4-carboxamide (0.25 g, 30%) as an off-white solid. MS ESI calculated for C₂₀H₃₁BrN₆O₄ [M+H]⁺, 499.16, found 499.30; ¹H NMR (400 MHz, CDCl₃) δ 6.83-6.81 (m, 2H), 6.71 (s, 1H), 5.39-5.26 (m, 2H), 5.15-5.12 (m, 1H), 4.53-4.50 (m, 2H), 4.39 (s, 1H), 3.97-3.92 (m, 4H), 3.95-3.78 (m, 3H), 3.66-3.62 (m, 1H), 3.51-3.39 (m, 3H), 3.10-3.08 (m, 1H), 2.25-2.22 (m, 2H), 1.85-1.82 (m, 4H), 1.43-1.41 (m, 2H), 1.25-1.22 (m, 2H).

### Step 5: 3-[2-(1-Ethyl-4,6-difluoro-1,3-benzodiazol-5-yl)ethynyl]-1-[(3S,5R)-5-(methoxymethyl)-1-(prop-2-enoyl)pyrrolidin-3-yl]-5-[[3-(morpholin-4-yl)propyl]amino]pyrazole-4-carboxamide

To a stirred solution of 3-bromo-1-[(3S,5R)-5-(methoxymethyl)-1-(prop-2-enoyl)pyrrolidin-3-yl]-5-[[3-(morpholin-4-yl)propyl]amino]pyrazole-4-carboxamide (0.15 g, 0.30 mmol), 1-ethyl-5-ethynyl-4,6-difluoro-1,3-benzodiazole (0.12 g, 0.60 mmol), Pd(PPh₃)₂Cl₂ (21.08 mg, 0.03 mmol) and CuI (11.44 mg, 0.06 mmol) in DMF (5.00 mL) was added TEA (91.18 mg, 0.90 mmol) dropwise at room temperature. The reaction mixture was degassed with argon for three times and stirred for 2 h at 90° C. The resulting mixture was cooled down and concentrated under reduced pressure. The residue was purified by silica gel Column chromatography, eluted with MeOH in DCM (0-10%) to afford the crude product. Then the crude product was purified by Prep-HPLC with the following conditions: Column: X-Bridge Prep C18 OBD Column, 19×150 mm 5 μm; Mobile Phase A: Water (10 mmoL/L NH₄HCO₃), Mobile Phase B: ACN; Flow rate: 20 mL/min; Gradient: 20 B to 50 B in 6 min; 210/254 nm. The fractions contained desired product were combined and concentrated to afford 3-[2-(1-ethyl-4,6-difluoro-1,3-benzodiazol-5-yl)ethynyl]-1-[(3S,5R)-5-(methoxymethyl)-1-(prop-2-enoyl)pyrrolidin-3-yl]-5-[[3-(morpholin-4-yl)propyl]amino]pyrazole-4-carboxamide (68 mg, 36%) as an off-white solid. MS ESI calculated for C₃₁H₃₈F₂N₅O₄ [M+H]⁺, 625.30, found 625.25; ¹H NMR (300 MHz, CDCl₃) δ 7.94 (s, 1H), 7.13-6.93 (m, 2H), 6.63-6.60 (m, 1H), 6.50-6.30 (m, 2H), 5.71-5.68 (m, 1H), 5.50-5.12 (m, 2H), 4.62-4.35 (m, 1H), 4.20-4.18 (m, 2H), 4.10-3.94 (m, 2H), 3.89-3.85 (m, 1H), 3.71-3.68 (m, 4H), 3.53-3.40 (m, 1H), 3.38-3.17 (m, 5H), 3.01-2.63 (m, 1H), 2.46 (d, J=6.5 Hz, 6H), 2.30-2.25 (m, 1H), 1.84-1.80 (m, 2H), 1.67 (s, 3H).

### Example 112: 3-[2-(1-Ethyl-4,6-difluoro-1,3-benzodiazol-5-yl)ethynyl]-5-[[2-(morpholin-4-yl)ethyl]amino]-1-[(3S)-1-(prop-2-enoyl)pyrrolidin-3-yl]pyrazole-4-carboxamide

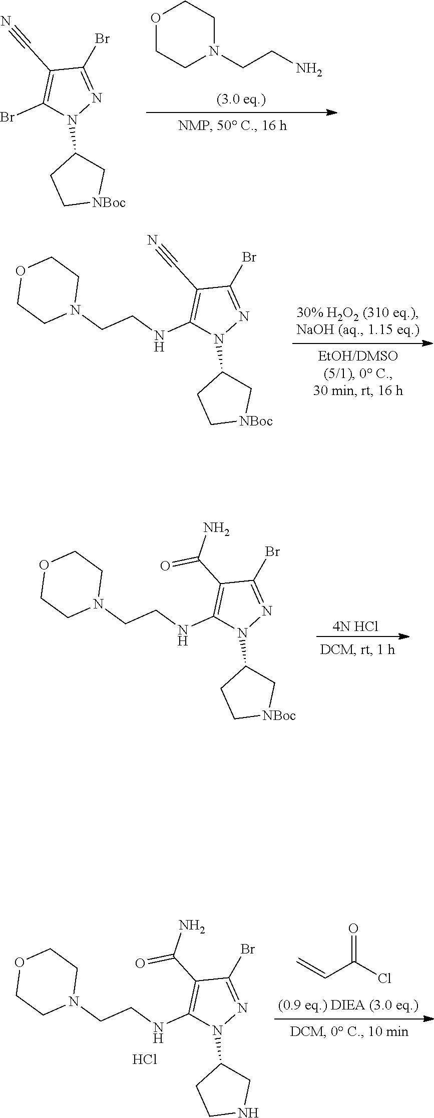

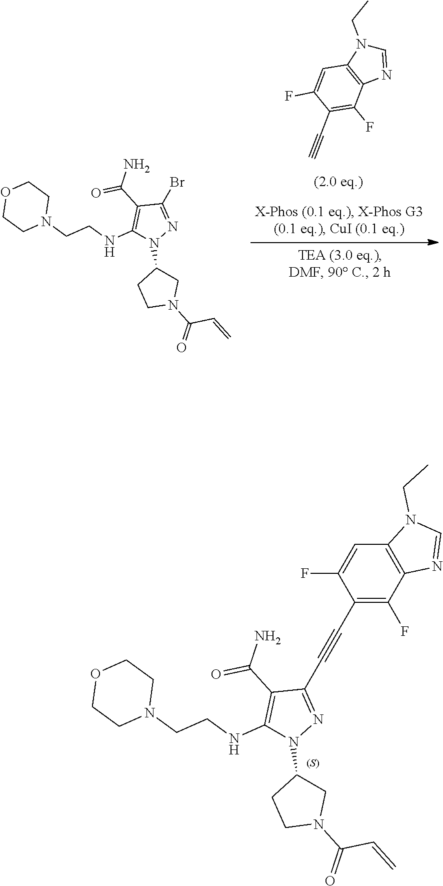

### Step 2: Tert-butyl (3S)-3-(3-bromo-4-carbamoyl-5-[[2-(morpholin-4-yl)ethyl]amino]pyrazol-1-yl)pyrrolidine-1-carboxylate

To a stirred solution of tert-butyl (2R,4S)-4-[3-bromo-4-cyano-5-[(cyclopropylmethyl)amino]pyrazol-1-yl]-2-(methoxymethyl)pyrrolidine-1-carboxylate (1.00 g, 2.20 mmol) in EtOH (10.00 mL) and DMSO (2.00 mL) were added NaOH (0.5 M in water) (0.11 mL, 2.70 mmol) and H₂O₂ (1.82 mL, 53.50 mmol, 30%) dropwise at 0° C. The reaction mixture was stirred for 30 min at 0° C. and 16 h at room temperature. The resulting mixture was diluted with water (20 mL) and extracted with EA (3×20 mL). The combined organic layers were washed with water (2×20 mL), dried over anhydrous Na₂SO₄ and filtered. The filtrate was concentrated under reduced pressure. The residue was purified by silica gel Column chromatography, eluted with EA in PE (20-70%). The fractions contained desired product were combined and concentrated to afford tert-butyl (3S)-3-(3-bromo-4-carbamoyl-5-[[2-(morpholin-4-yl)ethyl]amino]pyrazol-1-yl)pyrrolidine-1-carboxylate (0.37 g, 32%) as a yellow solid. MS ESI calculated for C₂₁H₃₅BrN₆O₅ [M+H]⁺, 487.16, 489.16, found 487.15, 489.15; ¹H NMR (400 MHz, CDCl₃) δ 6.92 (s, 1H), 6.55 (d, J=45.4 Hz, 1H), 5.49 (s, 1H), 5.09-4.81 (m, 1H), 3.92-3.67 (m, 8H), 3.56-3.41 (m, 1H), 3.29-3.27 (m, 2H), 3.00 (s, 1H), 2.59-2.54 (m, 7H), 2.28-2.25 (m, 1H), 1.50-1.47 (m, 9H), 1.29-1.25 (m, 2H).

### Step 3: 3-Bromo-5-[[2-(morpholin-4-yl)ethyl]amino]-1-[(3S)-pyrrolidin-3-yl]pyrazole-4-carboxamide hydrochloride

To a stirred solution of tert-butyl (3S)-3-(3-bromo-4-carbamoyl-5-[[2-(morpholin-4-yl)ethyl]amino]pyrazol-1-yl)pyrrolidine-1-carboxylate (0.36 g, 0.74 mmol) in DCM (4.00 mL) was added HCl (4 M in EA) (2.00 mL) dropwise at 0° C. The reaction mixture was stirred for 1 h at room temperature. The resulting mixture was concentrated under reduced pressure to afford 3-bromo-5-[[2-(morpholin-4-yl)ethyl]amino]-1-[(3S)-pyrrolidin-3-yl]pyrazole-4-carboxamide hydrochloride (0.32 g, 99%) as a light yellow solid which was used in the next step directly without further purification. MS ESI calculated for C₁₄H₂₃BrN₆O₂ [M+H]⁺, 387.16, 389.16, found 387.15, 389.15; ¹H NMR (400 MHz, CD₃OD) δ 5.51 (s, 2H), 5.45-5.39 (m, 1H), 4.17-3.89 (m, 3H), 3.86-3.69 (m, 2H), 3.73-3.65 (m, 1H), 3.65 (d, J=1.9 Hz, 1H), 3.65-3.57 (m, 1H), 3.61-3.54 (m, 1H), 3.57-3.48 (m, 1H), 3.41-3.36 (m, 2H), 3.30-3.18 (m, 2H), 2.70-2.65 (m, 1H), 2.59-2.56 (m, 1H), 2.37-2.31 (m, 1H), 2.06-2.03 (m, 1H), 1.64-1.61 (m, 1H), 1.33-1.16 (m, 1H).

### Step 4: 3-Bromo-5-[[2-(morpholin-4-yl)ethyl]amino]-1-[(3S)-1-(prop-2-enoyl)pyrrolidin-3-yl]pyrazole-4-carboxamide

To a stirred solution of 3-bromo-5-[[2-(morpholin-4-yl)ethyl]amino]-1-[(3S)-pyrrolidin-3-yl]pyrazole-4-carboxamide hydrochloride (0.32 g, 0.75 mmol) and DIEA (0.39 mL, 3.05 mmol) in DCM (4.00 mL) was added acryloyl chloride (61.52 mg, 0.68 mmol) dropwise at 0° C. under N₂ atmosphere. The reaction mixture was stirred for 10 min at 0° C. The resulting mixture was diluted with water (10 mL) and extracted with EA (3×20 mL). The combined organic layers were washed with water (20 mL), dried over anhydrous Na₂SO₄ and filtered. The filtrate was concentrated under reduced pressure. The residue was purified by silica gel Column chromatography, eluted with MeOH in DCM (0-10%). The fractions contained desired product were combined and concentrated to afford 3-bromo-5-[[2-(morpholin-4-yl)ethyl]amino]-1-[(3S)-1-(prop-2-enoyl)pyrrolidin-3-yl]pyrazole-4-carboxamide (0.20 g, 60%) as a yellow solid. MS ESI calculated for C₁₇H₂₅BrN₆O₃ [M+H]⁺, 441.12, 443.12, found 441.25, 443.25; ¹H NMR (300 MHz, CDCl₃) δ 6.96 (d, J=6.1 Hz, 1H), 6.58-6.41 (m, 1H), 6.45-6.36 (m, 1H), 5.79-5.67 (m, 1H), 5.46 (s, 1H), 5.04-4.93 (m, 1H), 4.11-3.83 (m, 3H), 3.88-3.51 (s, 1H), 3.31 (d, J=6.0 Hz, 2H), 2.68-2.65 (m, 2H), 2.64-2.62 (m, 1H), 2.55-2.52 (m, 4H), 2.52-2.28 (m, 1H), 1.60-1.57 (m, 1H), 1.49-1.46 (m, 1H), 1.28-1.27 (m, 1H), 1.26-1.24 (m, 1H), 0.93-0.82 (m, 1H).

### Step 5: 3-[2-(1-Ethyl-4,6-difluoro-1,3-benzodiazol-5-yl)ethynyl]-5-[[2-(morpholin-4-yl)ethyl]amino]-1-[(3S)-1-(prop-2-enoyl)pyrrolidin-3-yl]pyrazole-4-carboxamide

To a stirred mixture of 3-bromo-5-[[2-(morpholin-4-yl)ethyl]amino]-1-[(3S)-1-(prop-2-enoyl)pyrrolidin-3-yl]pyrazole-4-carboxamide (0.10 g, 0.23 mmol), 1-ethyl-5-ethynyl-4,6-difluoro-1,3-benzodiazole (93.44 mg, 0.45 mmol), CuI (4.32 mg, 0.02 mmol), X-Phos Pd G3 (19.18 mg, 0.02 mmol) and X-Phos (10.80 mg, 0.02 mmol) in DMF (2.00 mL) was added TEA (68.79 mg, 0.68 mmol) dropwise at room temperature. The reaction mixture was degassed with argon for three times and stirred for 2 h at 90° C. The resulting mixture was cooled down and concentrated under reduced pressure. The residue was purified by silica gel Column chromatography, eluted with MeOH in DCM (0-5%) to afford the crude product. Then the crude product was purified by Prep-HPLC with the following conditions: Column: X-Bridge Prep C18 OBD Column, 19×150 mm 5 μm; Mobile Phase A: Water (10 mmol/L NH₄HCO₃), Mobile Phase B: ACN; Flow rate: 20 mL/min; Gradient: 15 B to 50 B in 6 min. The fractions contained desired product were combined and concentrated to afford 3-[2-(1-ethyl-4,6-difluoro-1,3-benzodiazol-5-yl)ethynyl]-5-[[2-(morpholin-4-yl)ethyl]amino]-1-[(3S)-1-(prop-2-enoyl)pyrrolidin-3-yl]pyrazole-4-carboxamide (30.4 mg, 23%) as a white solid. MS ESI calculated for C₂₈H₃₂F₂N₈O₃[M+H]⁺, 567.26, found 567.25; ¹H NMR (400 MHz, CDCl₃) δ 7.96 (s, 1H), 7.21-6.95 (m, 2H), 6.76 (d, J=7.0 Hz, 1H), 6.62-6.33 (m, 2H), 5.79-5.65 (m, 1H), 5.44 (s, 1H), 5.10-5.07 (m, 1H), 4.25-4.20 (m, 2H), 4.17-3.86 (m, 3H), 3.78-3.76 (m, 5H), 3.31-3.27 (m, 2H), 2.83-2.27 (m, 8H), 1.60-1.56 (m, 2H), 1.33-1.27 (m, 1H).

### Example 113: 3-[2-(6-Chloro-7-fluoro-1-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

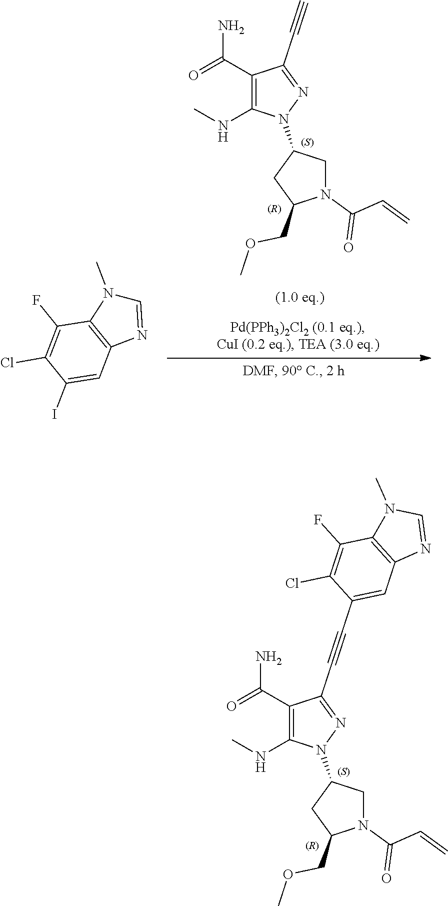

To a stirred mixture of 3-ethynyl-1-[(3S,5R)-5-(methoxymethyl)-1-(prop-2-enoyl)pyrrolidin-3-yl]-5-(methylamino)pyrazole-4-carboxamide (0.15 g, 0.45 mmol), 6-chloro-7-fluoro-5-iodo-1-methyl-1,3-benzodiazole (0.14 g, 0.45 mmol), CuI (17.24 mg, 0.09 mmol) and Pd(PPh₃)₂Cl₂ (31.77 mg, 0.04 mmol) in DMF (2.00 mL) was added TEA (0.14 g, 1.36 mmol). The reaction mixture was degassed with argon for three times and stirred for 2 h at 90° C. The resulting mixture was cooled down and concentrated under reduced pressure. The residue was purified by silica gel Column chromatography, eluted with MeOH in DCM (0-5%) to afford the crude product. Then the crude product was purified by Prep-HPLC with the following conditions: Column: X-Bridge Prep C18 OBD Column, 19×150 mm 5 μm; Mobile Phase A: Water (10 mmol/L NH₄HCO₃), Mobile Phase B: ACN; Flow rate: 20 mL/min; Gradient: 20 B to 50 B in 6 min. The fractions contained desired product were combined and concentrated to afford 3-[2-(6-chloro-7-fluoro-1-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (70.1 mg, 30%) as a white solid. MS ESI calculated for C₂₄H₂₅ClFN₇O₃[M+H]⁺, 514.17, found 514.15; ¹H NMR (300 MHz, CDCl₃) δ 7.89 (d, J=10.6 Hz, 2H), 7.11 (s, 1H), 6.83 (d, J=6.1 Hz, 1H), 6.56-6.35 (m, 2H), 5.72 (m, 1H), 5.61-5.46 (m, 2H), 4.60-4.57 (m, 1H), 4.18-3.88 (m, 6H), 3.41-3.58 (m, 1H), 3.56-3.45 (m, 3H), 3.02-2.98 (m, 3H), 2.81-2.64 (m, 1H), 2.41-2.35 (m, 1H).

### Example 114: 3-[2-(1,3-Benzoxazol-2-yl)ethynyl]-1-[(3S,5R)-5-(methoxymethyl)-1-(prop-2-enoyl)pyrrolidin-3-yl]-5-(methylamino)pyrazole-4-carboxamide

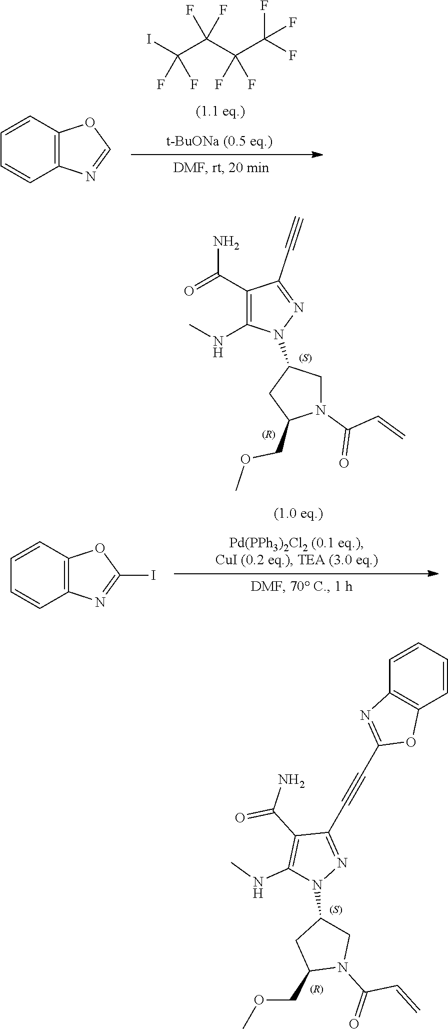

### Step 1: 2-Iodo-1,3-benzoxazole

To a stirred solution of benzoxazole (1 g, 8.40 mmol) in DMF (42.00 mL) were added 1,1,1,2,2,3,3,4,4-nonafluoro-4-iodobutane (1.59 mL, 9.24 mmol) and sodium 2-methylpropan-2-olate (0.40 g, 4.16 mmol) at room temperature. The reaction mixture was stirred for 20 min at room temperature. The resulting mixture was poured into water and extracted with EA (3×70 mL). The combined organic layers were washed with brine (5×50 mL), dried over anhydrous Na₂SO₄, filtered and concentrated under reduced pressure. The residue was purified by silica gel Column chromatography, eluted with 8% EA in PE. The fractions contained desired product were combined and concentrated to afford 2-iodo-1,3-benzoxazole (1.88 g, 91%) as a white solid. MS ESI calculated for C₇H₄INO [M+H]⁺, 245.93, found 245.95; ¹H NMR (400 MHz, CDCl₃) δ 7.74-7.68 (m, 1H), 7.57-7.52 (m, 1H), 7.35-7.28 (m, 2H).

### Step 2: 3-[2-(1,3-Benzoxazol-2-yl)ethynyl]-1-[(3S,5R)-5-(methoxymethyl)-1-(prop-2-enoyl)pyrrolidin-3-yl]-5-(methylamino)pyrazole-4-carboxamide

To a stirred mixture of 2-iodo-1,3-benzoxazole (0.11 g, 0.45 mmol), 3-ethynyl-1-[(3S,5R)-5-(methoxymethyl)-1-(prop-2-enoyl)pyrrolidin-3-yl]-5-(methylamino)pyrazole-4-carboxamide (0.15 g, 0.45 mmol), Pd(PPh₃)₂Cl₂ (31.51 mg, 0.05 mmol) and CuI (17.10 mg, 0.09 mmol) in DMF (1 mL) was added TEA (0.19 mL, 1.85 mmol). The reaction mixture was degassed with argon for three times and stirred for 1 h at 70° C. The resulting mixture was diluted with water (15 mL) and extracted with EA (3×30 mL). The combined organic layers were washed with brine (5×20 mL), dried over anhydrous Na₂SO₄ and filtered. The filtrate was concentrated under reduced pressure. The residue was purified by silica gel Column chromatography, eluted with CH₂Cl₂/MeOH (10/1). The fractions contained desired product were combined and concentrated. The crude product was purified by Prep-HPLC with the following conditions: Column: XBridge Prep C18 OBD Column, 19×150 mm 5 μm; Mobile Phase A: Water (10 mmol/L NH₄HCO₃), Mobile Phase B: ACN; Flow rate: 20 mL/min; Gradient: 15 B to 50 B in 6 min; 210/254 nm; RT: 5.75 min. The fractions contained desired product were combined and concentrated to afford 3-[2-(1,3-benzoxazol-2-yl)ethynyl]-1-[(3S,5R)-5-(methoxymethyl)-1-(prop-2-enoyl)pyrrolidin-3-yl]-5-(methylamino)pyrazole-4-carboxamide (0.07 g, 36%) as an off-white solid. MS ESI calculated for C₂₃H₂₄N₆O₄ [M+H]⁺, 448.47, found 448.19; ¹H NMR (400 MHz, CDCl₃) δ 7.81-7.79 (m, 1H), 7.58 (d, J=7.6 Hz, 1H), 7.50-7.41 (m, 2H), 6.86 (s, 1H), 6.68-6.44 (m, 2H), 5.76-5.72 (m, 1H), 5.56-5.52 (m, 2H), 4.60-4.58 (m, 1H), 4.41-3.91 (m, 3H), 3.54-3.39 (m, 4H), 3.07 (d, J=15.2 Hz, 3H), 2.98-2.65 (m, 1H), 2.37-2.32 (m, 1H).

### Example 115: 3-[2-(6-Fluoro-1,3-benzoxazol-5-yl)ethynyl]-1-[(3S,5R)-5-(methoxymethyl)-1-(prop-2-enoyl)pyrrolidin-3-yl]-5-(methylamino)pyrazole-4-carboxamide

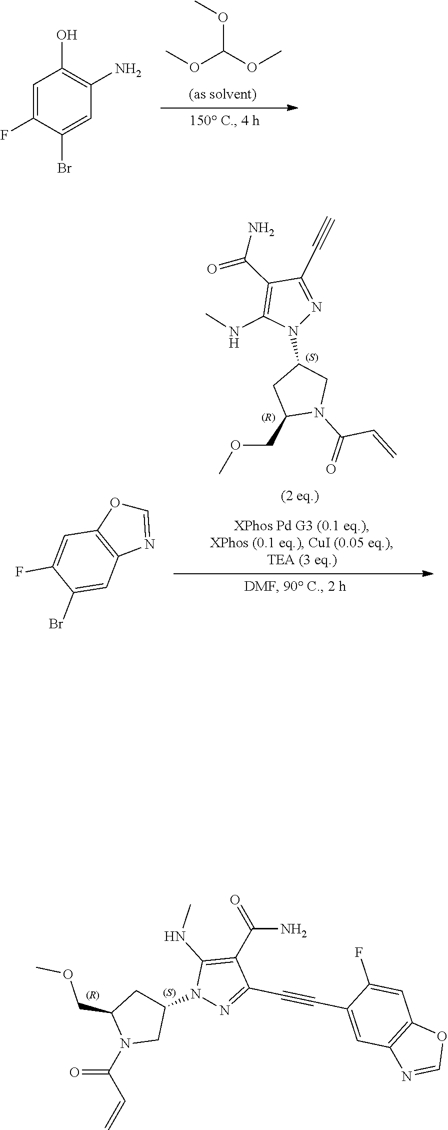

### Step 1: 5-Bromo-6-fluoro-1,3-benzoxazole

A solution of 2-amino-4-bromo-5-fluorophenol (1.70 g, 8.25 mmol) in trimethyl orthoformate (17.00 mL) was stirred for 4 h at 150° C. The resulting mixture was concentrated under reduced pressure. The residue was purified by silica gel Column chromatography, eluted with 8% EA in PE. The fractions contained desired product were combined and concentrated to afford 5-bromo-6-fluoro-1,3-benzoxazole (1.4 g, 78%) as a light pink solid. MS ESI calculated for C₇H₃BrFNO [M+H]⁺, 215.94, found 215.95; ¹H NMR (400 MHz, CDCl₃) δ 8.10 (s, 1H), 8.00 (d, J=6.3 Hz, 1H), 7.41 (d, J=7.4 Hz, 1H).

### Step 2: 3-[2-(6-Fluoro-1,3-benzoxazol-5-yl)ethynyl]-1-[(3S,5R)-5-(methoxymethyl)-1-(prop-2-enoyl)pyrrolidin-3-yl]-5-(methylamino)pyrazole-4-carboxamide

To a stirred mixture of 5-bromo-6-fluoro-1,3-benzoxazole (0.05 g, 0.23 mmol), 3-ethynyl-1-[(3S,5R)-5-(methoxymethyl)-1-(prop-2-enoyl)pyrrolidin-3-yl]-5-(methylamino)pyrazole-4-carboxamide (0.08 g, 0.23 mmol), XPhos Pd G₃ (39.20 mg, 0.05 mmol), XPhos (22 mg, 0.05 mmol) and CuI (2.20 mg, 0.01 mmol) in DMF (0.50 mL) was added TEA (0.10 mL, 0.95 mmol) dropwise at room temperature. The reaction mixture was degassed with argon for three times and stirred for 2 h at 90° C. The resulting mixture was diluted with water (15 mL) and extracted with EA (3×30 mL). The combined organic layers were washed with water (5×20 mL), dried over anhydrous Na₂SO₄ and filtered. The filtrate was concentrated under reduced pressure. The residue was purified by silica gel Column chromatography, eluted with CH₂Cl₂/MeOH (10/1). The fractions contained desired product were combined and concentrated. The crude product was purified by Prep-HPLC with the following conditions: Column: XBridge Prep C₁₈ OBD Column, 19×150 mm 5 μm; Mobile Phase A: Water (10 mmol/L NH₄HCO₃), Mobile Phase B: ACN; Flow rate: 20 mL/min; Gradient: 15 B to 50 B in 6 min; 210/254 nm; RT: 5.75 min. The fractions contained desired product were combined and concentrated to afford 3-[2-(6-fluoro-1,3-benzoxazol-5-yl)ethynyl]-1-[(3S,5R)-5-(methoxymethyl)-1-(prop-2-enoyl)pyrrolidin-3-yl]-5-(methylamino)pyrazole-4-carboxamide (0.07 g, 31%) as an off-white solid. MS ESI calculated for C₂₃H₂₃FN₆O₄ [M+H]⁺, 466.47, found 466.18; ¹H NMR (400 MHz, CDCl₃) δ 8.16 (s, 1H), 8.01 (t, J=16 Hz, 1H), 7.42 (d, J=8 Hz, 1H), 7.00 (s, 1H), 6.53-6.40 (m, 2H), 5.75-5.71 (m, 1H), 5.57-5.51 (m, 1H), 5.40-5.31 (m, 1H), 4.59-4.57 (m, 1H), 4.13-4.01 (m, 2H), 3.93-3.90 (m, 1H), 3.53-3.44 (m, 1H), 3.40 (d, J=4 Hz, 3H), 3.07-3.03 (m, 3H), 2.76-2.68 (m, 1H), 2.35-2.30 (m, 1H).

### Example 116: 3-[2-(1-Ethyl-4,6-difluoro-1,3-benzodiazol-5-yl)ethynyl]-5-[(2-hydroxyethyl)amino]-1-[(3S,5R)-5-(methoxymethyl)-1-(prop-2-enoyl)pyrrolidin-3-yl]pyrazole-4-carboxamide

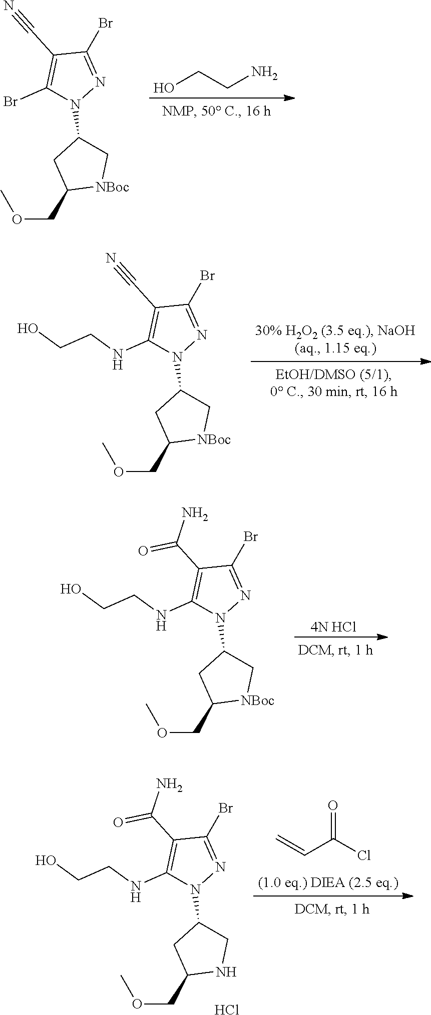

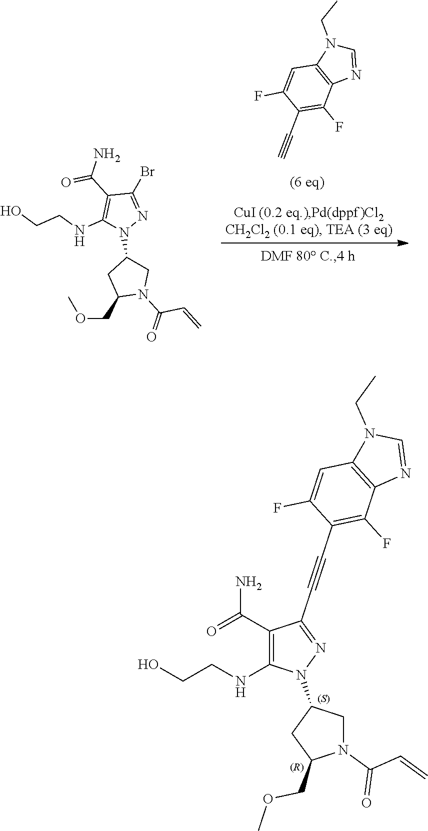

### Step 1: Tert-butyl (2R,4S)-4-[3-bromo-4-cyano-5-[(2-hydroxyethyl)amino]pyrazol-1-yl]-2-(methoxymethyl)pyrrolidine-1-carboxylate

To a stirred solution of tert-butyl (2R,4S)-4-(3,5-dibromo-4-cyanopyrazol-1-yl)-2-(methoxymethyl)pyrrolidine-1-carboxylate (1.00 g, 2.15 mmol) in NMP (10.00 mL, 0.10 mol) was added ethanolamine (0.39 g, 6.46 mmol). The reaction mixture was stirred for 16 h at 50° C. The resulting mixture was diluted with water (300 mL), extracted with EtOAc (3×300 mL), dried over anhydrous Na₂SO₄ and filtered. The filtrate was concentrated under reduced pressure to afford tert-butyl (2R,4S)-4-[3-bromo-4-cyano-5-[(2-hydroxyethyl)amino]pyrazol-1-yl]-2-(methoxymethyl)pyrrolidine-1-carboxylate (0.95 g, crude) which was used in the next step directly without further purification. MS ESI calculated for C₁₇H₂₈BrN₅O₅ [M+H]⁺, 444.12, found 444.20.

### Step 2: Tert-butyl (2R,4S)-4-[3-bromo-4-carbamoyl-5-[(2-hydroxyethyl)amino]pyrazol-1-yl]-2-(methoxymethyl)pyrrolidine-1-carboxylate

To a stirred solution tert-butyl (2R,4S)-4-[3-bromo-4-cyano-5-[(2-hydroxyethyl)amino]pyrazol-1-yl]-2-(methoxymethyl)pyrrolidine-1-carboxylate (0.95 g, 2.15 mmol) in DMSO (2.00 mL, 28.15 mmol) and EtOH (10.00 mL, 0.17 mol) was added H₂O₂ (1.67 mL, 71.68 mmol, 30%) and NaOH (4.95 mL, 2.47 mmol, 0.5 M) dropwise at 0° C. The reaction mixture was stirred for 30 min at 0° C. and 16 h at room temperature. The resulting mixture was diluted with water (300 mL), extracted with EtOAc (3×300 mL), dried over anhydrous Na₂SO₄ and filtered. The filtrate was concentrated under reduced pressure. The residue was purified by silica gel Column chromatography, eluted with PE/EtOAc (0-50%). The fractions contained desired product were combined and concentrated to afford tert-butyl (2R,4S)-4-[3-bromo-4-carbamoyl-5-[(2-hydroxyethyl)amino]pyrazol-1-yl]-2-(methoxymethyl)pyrrolidine-1-carboxylate (0.65 g, 65%) as an off-white oil. MS ESI calculated for C₁₇H₂₈BrN₅O₅ [M+H]⁺, 462.13, found 462.15.

### Step 3: 3-Bromo-5-[(2-hydroxyethyl)amino]-1-[(3S,5R)-5-(methoxymethyl)pyrrolidin-3-yl]pyrazole-4-carboxamide

To a stirred solution of tert-butyl (2R,4S)-4-[3-bromo-4-carbamoyl-5-[(2-hydroxyethyl)amino]pyrazol-1-yl]-2-(methoxymethyl)pyrrolidine-1-carboxylate (0.65 g, 1.40 mmol) in DCM (7.00 mL) was added HCl (4 M in EA) (7.73 mL, 30.93 mmol) dropwise at 0° C. The reaction mixture was stirred for 1 h at room temperature. The resulting mixture was concentrated under reduced pressure to afford 3-bromo-5-[(2-hydroxyethyl)amino]-1-[(3S,5R)-5-(methoxymethyl)pyrrolidin-3-yl]pyrazole-4-carboxamide (0.5 g, crude) which was used in the next step directly without further purification. MS ESI calculated for C₁₂H₂₀BrN₅O₃ [M+H−HCl]⁺, 362.07, found 362.10.

### Step 4: 3-Bromo-5-[(2-hydroxyethyl)amino]-1-[(3S,5R)-5-(methoxymethyl)-1-(prop-2-enoyl)pyrrolidin-3-yl]pyrazole-4-carboxamide

To a stirred mixture of 3-bromo-5-[(2-hydroxyethyl)amino]-1-[(3S,5R)-5-(methoxymethyl)pyrrolidin-3-yl]pyrazole-4-carboxamide (0.5 g, 1.38 mmol) and DIEA (0.60 mL, 4.65 mmol) in DCM (5.00 mL) was added acryloyl chloride (5.02 mL, 1.25 mmol) dropwise at 0° C. The reaction mixture was stirred for 1 h at room temperature. The resulting mixture was quenched with H₂O (1 mL) and concentrated under reduced pressure. The residue was purified by silica gel Column chromatography, eluted with CH₂Cl₂/MeOH (0-10%). The fractions contained desired product were combined and concentrated to afford 3-bromo-5-[(2-hydroxyethyl)amino]-1-[(3S,5R)-5-(methoxymethyl)-1-(prop-2-enoyl)pyrrolidin-3-yl]pyrazole-4-carboxamide (0.55 g, 95%) as an off-white oil. MS ESI calculated for C₁₅H₂₂BrN₅O₄ [M+H]⁺, 416.09, found 416.15; ¹H NMR (300 MHz, CDCl₃) δ 6.73 (s, 1H), 6.57-6.33 (m, 2H), 5.71-5.68 (m, 1H), 5.31 (s, 2H), 4.03-3.81 (m, 2H), 3.68-3.62 (m, 5H), 3.42-3.31 (m, 4H), 3.11-3.08 (m, 3H).

### Step 5: 3-[2-(1-Ethyl-4,6-difluoro-1,3-benzodiazol-5-yl)ethynyl]-5-[(2-hydroxyethyl)amino]-1-[(3S,5R)-5-(methoxymethyl)-1-(prop-2-enoyl)pyrrolidin-3-yl]pyrazole-4-carboxamide

To a stirred mixture of 3-bromo-5-[(2-hydroxyethyl)amino]-1-[(3S,5R)-5-(methoxymethyl)-1-(prop-2-enoyl)pyrrolidin-3-yl]pyrazole-4-carboxamide (0.19 g, 0.45 mmol), 1-ethyl-5-ethynyl-4,6-difluoro-1,3-benzodiazole (0.56 g, 2.73 mmol), CuI (17.39 mg, 0.09 mmol) and Pd(dppf)Cl₂CH₂Cl₂ (37.18 mg, 0.05 mmol) in DMF (6.00 mL) was added TEA (0.14 g, 1.36 mmol) dropwise at room temperature. The reaction mixture was degassed with argon for three times and stirred for 4 h at 80° C. The resulting mixture was concentrated under reduced pressure. The residue was purified by silica gel Column chromatography, eluted with CH₂Cl₂/MeOH (0-10%) to afford the crude. The crude was purified by reverse flash chromatography with the following conditions: Column: XBridge Prep C18 OBD Column, 19×150 mm 5 μm; Mobile Phase A: Water (10 mmol/L NH₄HCO₃), Mobile Phase B: ACN; Flow rate: 20 mL/min; Gradient: 5 B to 40 B in 6 min; 210/254 nm; RT: 5.58 min. The fractions contained desired product were combined and concentrated to afford 3-[2-(1-ethyl-4,6-difluoro-1,3-benzodiazol-5-yl)ethynyl]-5-[(2-hydroxyethyl)amino]-1-[(3S,5R)-5-(methoxymethyl)-1-(prop-2-enoyl)pyrrolidin-3-yl]pyrazole-4-carboxamide (62.2 mg, 25%) as a white solid. MS ESI calculated for C₂₆H₂₉F₂N₇O₄[M+H]⁺, 542.22, found 542.20; ¹H NMR (300 MHz, DMSO-d₆) δ 8.46 (s, 1H), 7.72 (d, J=9.2 Hz, 1H), 7.59 (s, 1H), 6.81-6.51 (m, 3H), 6.18 (d, J=16.8 Hz, 1H), 5.70-5.65 (m, 1H), 5.34-5.23 (m, 1H), 4.90-4.88 (m, 1H), 4.48 (d, J=41.8 Hz, 1H), 4.31 (q, J=7.2 Hz, 2H), 4.04-4.01 (m, 1H), 3.86 (m, 2H), 3.61-3.43 (m, 4H), 2.46-2.41 (m, 1H), 2.33-2.30 (m, 1H), 1.42 (t, J=7.2 Hz, 3H).

### Example 117: 3-[2-(1-Ethyl-4,6-difluoro-1,3-benzodiazol-5-yl)ethynyl]-5-[(2-methoxyethyl)amino]-1-[(3S,5R)-5-(methoxymethyl)-1-(prop-2-enoyl)pyrrolidin-3-yl]pyrazole-4-carboxamide

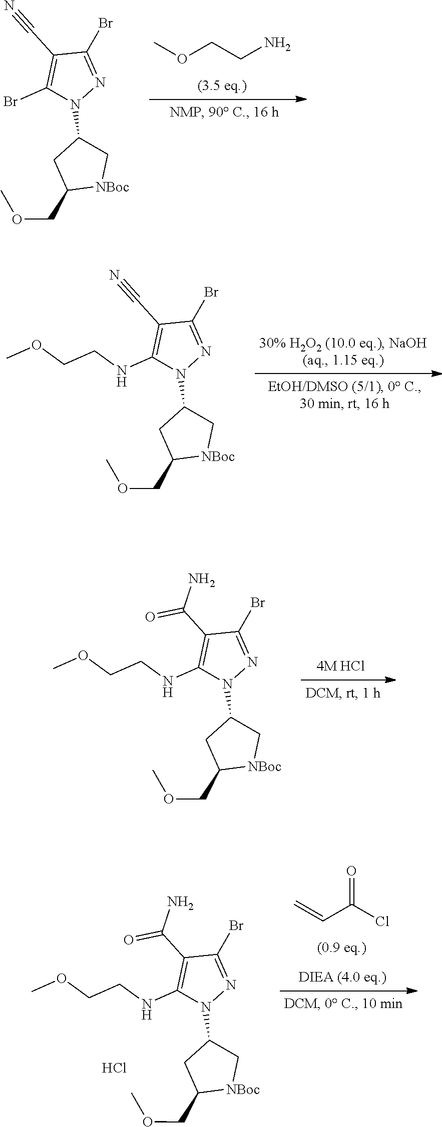

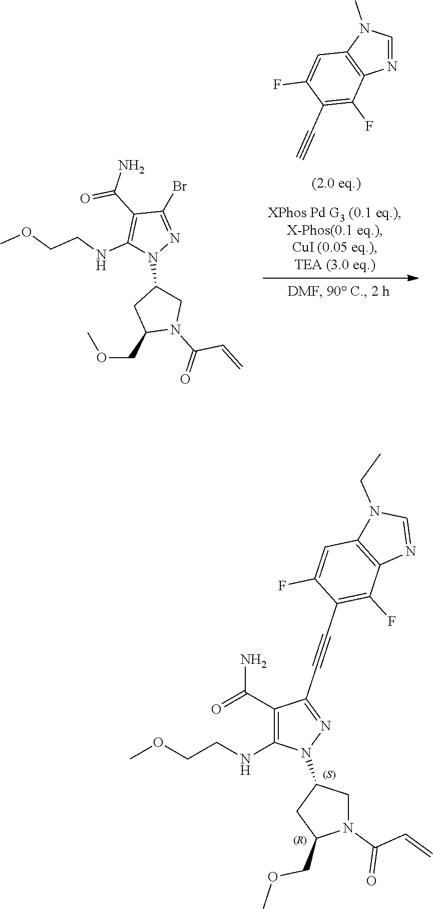

### Step 1: Tert-butyl (2R,4S)-4-[3-bromo-4-cyano-5-[(2-methoxyethyl)amino]pyrazol-1-yl]-2-(methoxymethyl)pyrrolidine-1-carboxylate

To a stirred solution of tert-butyl (2R,4S)-4-(3,5-dibromo-4-cyanopyrazol-1-yl)-2-(methoxymethyl)pyrrolidine-1-carboxylate (1.00 g, 2.15 mmol) in NMP (10.00 mL) was added ethanamine 2-methoxy (0.57 g, 7.58 mmol). The reaction mixture was stirred for 16 h at 90° C. The resulting mixture was cooled down, diluted with water (30 mL) and extracted with EtOAc (3×30 mL). The combined organic layers were washed with brine (3×30 mL), dried over anhydrous Na₂SO₄ and filtered. The filtrate was concentrated under reduced pressure to afford tert-butyl (2R,4S)-4-[3-bromo-4-cyano-5-[(2-methoxyethyl)amino]pyrazol-1-yl]-2-(methoxymethyl)pyrrolidine-1-carboxylate (0.98 g, 99%) as a yellow oil which was used in the next step directly without further purification. MS ESI calculated for C₁₈H₂₈BrN₅O₄ [M+H−56]⁺, 402.13, 404.13, found 401.95, 403.95; ¹H NMR (300 MHz, CDCl₃) δ 6.37 (s, 1H), 4.99-4.96 (m, 1H), 4.14-4.12 (m, 1H), 3.78-3.66 (m, 4H), 3.62-3.59 (m, 2H), 3.45-3.59 (m, 7H), 2.66-2.63 (m, 1H), 2.19-2.15 (m, 1H), 1.29 (s, 9H).

### Step 2: Tert-butyl (2R,4S)-4-[3-bromo-4-carbamoyl-5-[(2-methoxyethyl)amino]pyrazol-1-yl]-2-(methoxymethyl)pyrrolidine-1-carboxylate

To a stirred solution of tert-butyl (2R,4S)-4-[3-bromo-4-cyano-5-[(2-methoxyethyl)amino]pyrazol-1-yl]-2-(methoxymethyl)pyrrolidine-1-carboxylate (0.98 g, 2.13 mmol) and NaOH (98.34 mg, 2.45 mmol, 0.5 M) in EtOH (10.00 mL) and DMSO (2.00 mL) was added H₂O₂ (0.72 g, 21.38 mmol, 30%) dropwise at 0° C. The reaction mixture was stirred for 30 min at 0° C. and 16 h at room temperature. The resulting mixture was diluted with water (30 mL) and extracted with EtOAc (3×30 mL). The combined organic layers were washed with brine (3×30 mL), dried over anhydrous Na₂SO₄ and filtered. The filtrate was concentrated under reduced pressure. The residue was purified by silica gel Column chromatography, eluted with EA in PE (0-64%). The fractions contained desired product were combined and concentrated to afford tert-butyl (2R,4S)-4-[3-bromo-4-carbamoyl-5-[(2-methoxyethyl)amino]pyrazol-1-yl]-2-(methoxymethyl)pyrrolidine-1-carboxylate (0.72 g, 70%) as an off-white solid. MS ESI calculated for C₁₈H₃₀BrN₅O₅ [M+H]⁺, 420.15, 422.15, found, 419.95, 421.95; ¹H NMR (300 MHz, CDCl₃) δ 6.57 (s, 1H), 5.17 (s, 1H), 4.15 (s, 1H), 3.87-3.45 (m, 5H), 3.40-3.35 (m, 8H), 2.62-2.59 (m, 1H), 2.23-2.21 (m, 1H), 1.66-1.10 (m, 9H).

### Step 3: 3-Bromo-5-[(2-methoxyethyl)amino]-1-[(3S,5R)-5-(methoxymethyl)pyrrolidin-3-yl]pyrazole-4-carboxamide hydrochloride

To a stirred solution of tert-butyl (2R,4S)-4-[3-bromo-4-carbamoyl-5-[(2-methoxyethyl)amino]pyrazol-1-yl]-2-(methoxymethyl)pyrrolidine-1-carboxylate (0.72 g, 1.51 mmol) in DCM (8.00 mL) was added HCl (gas) in 1,4-dioxane (8.00 mL, 140.14 mmol, 4 M) dropwise at 0° C. The reaction mixture was stirred for 1 h at room temperature. The resulting mixture was concentrated under reduced pressure to afford 3-bromo-5-[(2-methoxyethyl)amino]-1-[(3S,5R)-5-(methoxymethyl)pyrrolidin-3-yl]pyrazole-4-carboxamide hydrochloride (0.61 g, 98%) as an off-white solid which was used in the next step directly without further purification. MS ESI calculated for C₁₃H₂₂BrN₅O₃ [M+H]⁺, 376.10, 378.10, found 376.10, 378.10.

### Step 4: 3-Bromo-5-[(2-methoxyethyl)amino]-1-[(3S,5R)-5-(methoxymethyl)-1-(prop-2-enoyl)pyrrolidin-3-yl]pyrazole-4-carboxamide 4-chloro-1-ethyl-5-iodo-2-methyl-1,3-benzodiazole

To a stirred solution of 3-bromo-5-[(2-methoxyethyl)amino]-1-[(3S,5R)-5-(methoxymethyl)pyrrolidin-3-yl]pyrazole-4-carboxamide hydrochloride (0.51 g, 1.23 mmol) in DCM (6.00 mL) were added DIEA (0.63 g, 4.94 mmol) and acryloyl chloride (4.45 mL, 1.11 mmol) dropwise at 0° C. under nitrogen atmosphere. The reaction mixture was diluted with water (30 mL) and extracted with CH₂Cl₂ (3×30 mL). The combined organic layers were washed with brine (30 mL), dried over anhydrous Na₂SO₄ and filtered. The filtrate was concentrated under reduced pressure. The residue was purified by silica gel Column chromatography, eluted with MeOH in DCM (0-4%). The fractions contained desired product were combined and concentrated to afford 3-bromo-5-[(2-methoxyethyl)amino]-1-[(3S,5R)-5-(methoxymethyl)-1-(prop-2-enoyl)pyrrolidin-3-yl]pyrazole-4-carboxamide (0.47 g, 88%) as an off-white solid. MS ESI calculated for C₁₆H₂₄BrN₅O₄ [M+H]⁺, 430.11, 432.11, found 430.20, 432.20; ¹H NMR (300 MHz, CDCl₃) δ 6.92 (s, 1H), 6.56-6.33 (m, 2H), 5.72-5.69 (m, 1H), 5.55-5.11 (m, 2H), 4.69-4.29 (m, 1H), 4.06-3.77 (m, 3H), 3.57 (q, J=5.6 Hz, 2H), 3.51-3.28 (m, 9H), 2.72-2.50 (m, 1H), 2.46-2.14 (m, 1H).

### Step 5: 3-[2-(1-Ethyl-4,6-difluoro-1,3-benzodiazol-5-yl)ethynyl]-5-[(2-methoxyethyl)amino]-1-[(3S,5R)-5-(methoxymethyl)-1-(prop-2-enoyl)pyrrolidin-3-yl]pyrazole-4-carboxamide

To a stirred solution of 3-bromo-5-[(2-methoxyethyl)amino]-1-[(3S,5R)-5-(methoxymethyl)-1-(prop-2-enoyl)pyrrolidin-3-yl]pyrazole-4-carboxamide (0.15 g, 0.34 mmol) and 1-ethyl-5-ethynyl-4,6-difluoro-1,3-benzodiazole (0.14 g, 0.69 mmol) in DMF (2.50 mL) were added XPhos Pd G₃ (29.51 mg, 0.04 mmol), X-Phos (16.62 mg, 0.03 mmol), CuI (3.32 mg, 0.01 mmol) and TEA (0.10 g, 1.04 mmol) dropwise at room temperature. The reaction mixture was degassed with argon for three times and stirred for 2 h at 90° C. The resulting mixture was cooled and concentrated under reduced pressure. The residue was purified by silica gel Column chromatography, eluted with MeOH in DCM (0-4%) to afford crude product. The crude product was purified by Prep-HPLC with the following conditions: Column: XBridge Prep Phenyl OBD Column, 19×150 mm 5 μm 13 nm; Mobile Phase A: Water (10 mmol/L NH₄HCO₃), Mobile Phase B: ACN; Flow rate: 20 mL/min; Gradient: 25 B to 50 B in 6 min, 210/254 nm; RT: 5.58 min. The fractions contained desired product were combined and concentrated to afford 3-[2-(1-ethyl-4,6-difluoro-1,3-benzodiazol-5-yl)ethynyl]-5-[(2-methoxyethyl)amino]-1-[(3S,5R)-5-(methoxymethyl)-1-(prop-2-enoyl)pyrrolidin-3-yl]pyrazole-4-carboxamide (99.4 mg, 50%) as an off-white solid. MS ESI calculated for C₂₇H₃₁F₂N₇O₄[M+H]⁺, 556.25, found 556.20; ¹H NMR (300 MHz, CDCl₃) δ 7.96 (s, 1H), 7.06-7.02 (m, 2H), 6.76 (s, 1H), 6.62-6.33 (m, 2H), 5.72 (dd, J=7.9, 4.5 Hz, 1H), 5.62-5.24 (m, 2H), 4.66-4.38 (m, 1H), 4.22 (q, J=7.3 Hz, 2H), 4.08-4.42 (m, 2H), 3.89-3.85 (m, 1H), 3.61 (q, J=5.8 Hz, 2H), 3.54-3.29 (m, 9H), 3.01-2.61 (m, 1H), 2.33-2.30 (m, 1H), 1.57 (t, J=7.3 Hz, 3H).

### Example 118: 5-[(Cyclopropylmethyl)amino]-3-[2-(1-ethyl-4,6-difluoro-1,3-benzodiazol-5-yl)ethynyl]-1-[(3S)-1-(prop-2-enoyl)pyrrolidin-3-yl]pyrazole-4-carboxamide

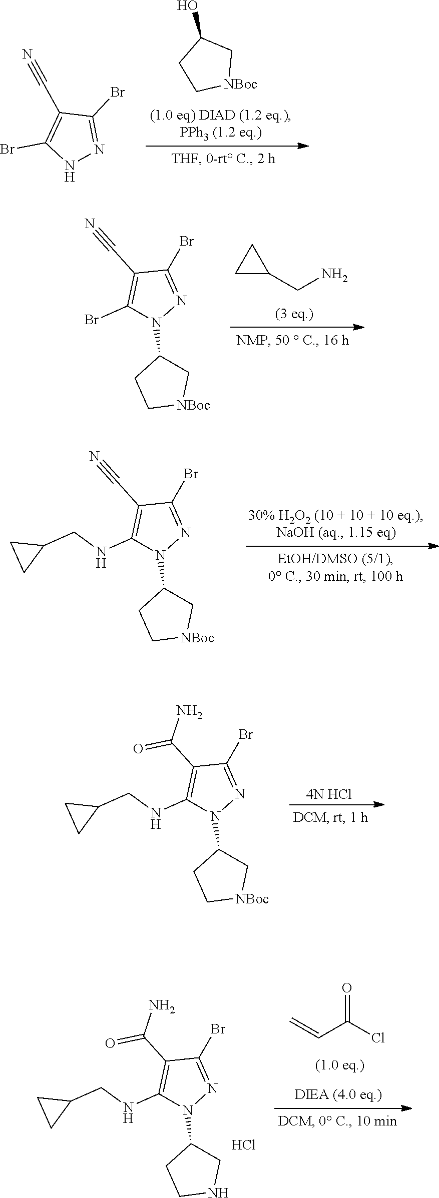

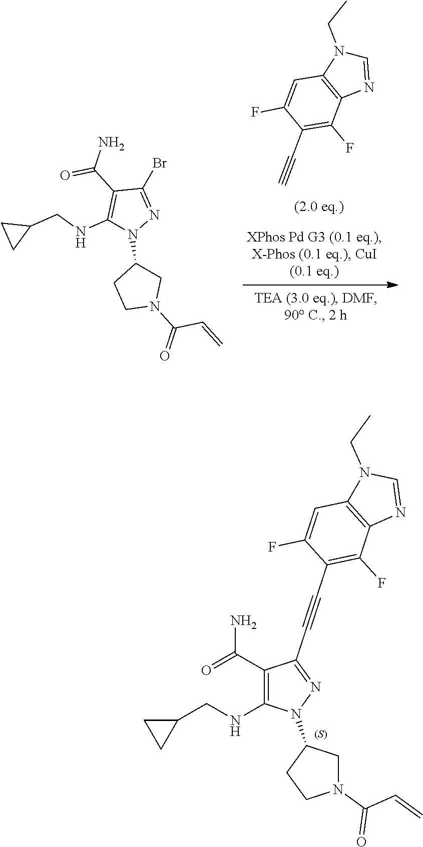

### Step 1: Tert-butyl (3S)-3-(3,5-dibromo-4-cyanopyrazol-1-yl)pyrrolidine-1-carboxylate

To a stirred solution of 3,5-dibromo-1H-pyrazole-4-carbonitrile (6.90 g, 27.50 mmol) and tert-butyl (3R)-3-hydroxypyrrolidine-1-carboxylate (5.15 g, 27.50 mmol) in THF (120.00 mL) were added PPh₃ (8.66 g, 33.00 mmol) and DIAD (6.67 g, 33.00 mmol) dropwise at 0° C. under nitrogen atmosphere. The reaction mixture was stirred for 2 h at room temperature. The resulting mixture was concentrated under reduced pressure. The residue was diluted with water (100 mL) and extracted with EtOAc (3×150 mL). The combined organic layers were washed with brine (100 mL), dried over anhydrous Na₂SO₄ and filtered. The filtrate was concentrated under reduced pressure. The residue was purified by silica gel Column chromatography, eluted with EA in PE (0-21%). The fractions contained desired product were combined and concentrated to afford tert-butyl (3S)-3-(3,5-dibromo-4-cyanopyrazol-1-yl)pyrrolidine-1-carboxylate (11.4 g, 98%) as an off-white solid. MS ESI calculated for C₁₃H₁₆Br₂N₄O₂ [M+H−56]⁺, 362.96, 364.96, found 362.90, 364.90; ¹H NMR (400 MHz, CDCl₃) δ 6.37-6.35 (m, 1H), 50.5-4.92 (m, 1H), 3.78-3.73 (m, 2H), 3.52-3.49 (m, 1H), 2.70-2.16 (m, 1H), 1.69-1.68 (m, 1H), 1.48-1.26 (m, 9H).

### Step 2: Tert-butyl (3S)-3-[3-bromo-4-cyano-5-[(cyclopropylmethyl)amino]pyrazol-1-yl]pyrrolidine-1-carboxylate

To a stirred mixture of tert-butyl (3S)-3-(3,5-dibromo-4-cyanopyrazol-1-yl)pyrrolidine-1-carboxylate (50.00 mg, 0.12 mmol) in NMP (0.5 mL) was added 1-cyclopropylmethanamine (16.93 mg, 0.24 mmol) dropwise at room temperature. The reaction mixture was stirred for 16 h at 50° C. under argon atmosphere. The resulting mixture was diluted with water (100 mL) and extracted with EtOAc (3×100 mL). The combined organic layers were washed with brine (8×50 mL), dried over anhydrous Na₂SO₄ and filtered. The filtrate was concentrated under reduced pressure to afford tert-butyl (3S)-3-[3-bromo-4-cyano-5-[(cyclopropylmethyl)amino]pyrazol-1-yl]pyrrolidine-1-carboxylate (0.90 g, 92%) as a yellow oil which was used in the next step directly without further purification. MS ESI calculated for C₁₇H₂₄BrN₅O₂ [M−H]⁻, 408.11, found 408.00.

### Step 3: Tert-butyl (3S)-3-[3-bromo-4-carbamoyl-5-[(cyclopropylmethyl)amino]pyrazol-1-yl]pyrrolidine-1-carboxylate

To a stirred mixture of tert-butyl (3S)-3-[3-bromo-4-cyano-5-[(cyclopropylmethyl)amino]pyrazol-1-yl]pyrrolidine-1-carboxylate (0.85 g, 2.07 mmol) in EtOH (7.00 mL) and DMSO (1.40 mL) were added NaOH (0.10 g, 2.38 mmol, 0.5 M) and H₂O₂ (2.35 g, 20.72 mmol, 30%) dropwise at 0° C. under nitrogen atmosphere. The reaction mixture was stirred for 0.5 h at 0° C. and 100 h at room temperature under nitrogen atmosphere. The resulting mixture was quenched by the addition of water (10 mL) at 0° C. and extracted with EtOAc (3×20 mL). The combined organic layers were washed with brine (3×50 mL), dried over anhydrous Na₂SO₄ and filtered. The filtrate was concentrated under reduced pressure. The residue was purified by silica gel Column chromatography, eluted with PE/EtOAc (1/2). The fractions contained desired product were combined and concentrated. The residue was purified by reverse flash chromatography with the following conditions: Column, C18 silica gel; mobile phase, MeCN in water (10 mmol/L, NH₄HCO₃), 10% to 50% gradient in 30 min; detector, UV 254 nm. The fractions contained desired product were combined and concentrated to afford tert-butyl (3S)-3-[3-bromo-4-carbamoyl-5-[(cyclopropylmethyl)amino]pyrazol-1-yl]pyrrolidine-1-carboxylate (0.18 g, 20%) as an off-white semi-solid. MS ESI calculated for C₁₇H₂₆BrN₅O₃ [M+H]⁺, 428.13, found 428.15; ¹H NMR (400 MHz, CDCl₃) δ 6.76 (s, 1H), 6.61 (s, 1H), 4.83 (d, J=7.8 Hz, 1H), 3.69-3.65 (m, 1H), 3.47-3.45 (m, 1H), 3.05 (t, J=6.6 Hz, 2H), 2.52 (s, 1H), 2.19-2.07 (m, 1H), 1.64 (s, 2H), 1.49 (s, 9H), 1.28-1.26 (m, 1H), 1.09 (s, 1H), 0.64-0.57 (m, 2H), 0.27-0.25 (m, 2H).

### Step 4: 3-Bromo-5-[(cyclopropylmethyl)amino]-1-[(3S)-pyrrolidin-3-yl]pyrazole-4-carboxamide hydrochloride

To a stirred mixture of tert-butyl (3S)-3-[3-bromo-4-carbamoyl-5-[(cyclopropylmethyl)amino]pyrazol-1-yl]pyrrolidine-1-carboxylate (0.18 g, 0.42 mmol) in DCM (2.00 mL) was added HCl (4 M in EtOAc) (2.00 mL) dropwise at 0° C. The reaction mixture was stirred for 1 h at room temperature. The resulting mixture was concentrated under reduced pressure to afford 3-bromo-5-[(cyclopropylmethyl)amino]-1-[(3S)-pyrrolidin-3-yl]pyrazole-4-carboxamide hydrochloride (0.20 g, 99%) as an off-white solid which was used in the next step directly without further purification. MS ESI calculated for C₁₂H₁₈BrN₅O [M+H]⁺, 328.07, 330.07, found 328.00, 330.00.

### Step 5: 3-Bromo-5-[(cyclopropylmethyl)amino]-1-[(3S)-1-(prop-2-enoyl)pyrrolidin-3-yl]pyrazole-4-carboxamide

To a stirred mixture of 3-bromo-5-[(cyclopropylmethyl)amino]-1-[(3S)-pyrrolidin-3-yl]pyrazole-4-carboxamide hydrochloride (0.18 g, 0.49 mmol) and DIEA (28.35 mg, 0.22 mmol) in DCM (4.50 mL) was added acryloyl chloride (1.82 mL, 0.45 mmol) dropwise at 0° C. The reaction mixture was stirred for 10 min at 0° C. under nitrogen atmosphere. The resulting mixture was quenched with water (10 mL) at 0° C. and extracted with CH₂Cl₂ (3×10 mL). The combined organic layers was dried over anhydrous Na₂SO₄ and filtered. The filtrate was concentrated under reduced pressure. The residue was purified by silica gel Column chromatography, eluted with CH₂Cl₂/MeOH (10/1). The fractions contained desired product were combined and concentrated to afford 3-bromo-5-[(cyclopropylmethyl)amino]-1-[(3S)-1-(prop-2-enoyl)pyrrolidin-3-yl]pyrazole-4-carboxamide (0.15 g, 79%) as a light yellow solid. MS ESI calculated for C₁₅H₂₀BrN₅O₂ [M+H]⁺, 382.09, found 382.00.

### Step 6: 5-[(Cyclopropylmethyl)amino]-3-[2-(1-ethyl-4,6-difluoro-1,3-benzodiazol-5-yl)ethynyl]-1-[(3S)-1-(prop-2-enoyl)pyrrolidin-3-yl]pyrazole-4-carboxamide

To a stirred mixture of 3-bromo-5-[(cyclopropylmethyl)amino]-1-[(3S)-1-(prop-2-enoyl)pyrrolidin-3-yl]pyrazole-4-carboxamide (0.15 g, 0.39 mmol) and 1-ethyl-5-ethynyl-4,6-difluoro-1,3-benzodiazole (0.16 mg, 0.78 mmol) in DMF (4.00 mL) were added X-Phos (18.71 mg, 0.04 mmol), XPhos Pd G3 (33.21 mg, 0.04 mmol), CuI (7.47 mg, 0.04 mmol) and TEA (0.12 g, 1.18 mmol) dropwise at room temperature. The reaction mixture was degassed with argon for three times and stirred for 2 h at 90° C. The resulting mixture was cooled down and concentrated under reduced pressure. The residue was purified by silica gel Column chromatography, eluted with CH₂Cl₂/MeOH (10/1). The fractions contained desired product were combined and concentrated. The residue was purified by reverse flash chromatography with the following conditions: Column: SunFire Prep C18 OBD Column, 19×150 mm 5 μm 10 nm; Mobile Phase A: Water (0.05% FA), Mobile Phase B: ACN; Flow rate: 20 mL/min; Gradient: 25 B to 50 B in 6 min; 210/254 nm; RT: 5.58 min. The fractions contained desired product were combined and concentrated to afford 5-[(cyclopropylmethyl)amino]-3-[2-(1-ethyl-4,6-difluoro-1,3-benzodiazol-5-yl)ethynyl]-1-[(3S)-1-(prop-2-enoyl)pyrrolidin-3-yl]pyrazole-4-carboxamide (80.6 mg, 39%) as an off-white solid. MS ESI calculated for C₂₆H₂₇F₂N₇O₂[M+H]⁺, 508.32, found 508.30; ¹H NMR (300 MHz, CDCl₃) δ 7.97 (s, 1H), 7.23-6.98 (m, 2H), 6.61-6.37 (m, 3H), 5.80-5.68 (m, 1H), 5.45 (s, 1H), 5.01 (dd, J=13.2, 6.8 Hz, 1H), 4.32-3.83 (m, 5H), 3.75-3.68 (m, 1H), 3.05 (q, J=6.9 Hz, 2H), 2.84-2.50 (m, 1H), 2.37 (s, 1H), 1.58 (t, J=7.3 Hz, 3H), 1.12 (s, 1H), 0.67-0.57 (m, 2H), 0.29-0.27 (m, 2H).

### Example 119: 3-[2-(1-Ethyl-4,6-difluoro-1,3-benzodiazol-5-yl)ethynyl]-5-[(2-methoxyethyl)amino]-1-[(3S)-1-(prop-2-enoyl)pyrrolidin-3-yl]pyrazole-4-carboxamide

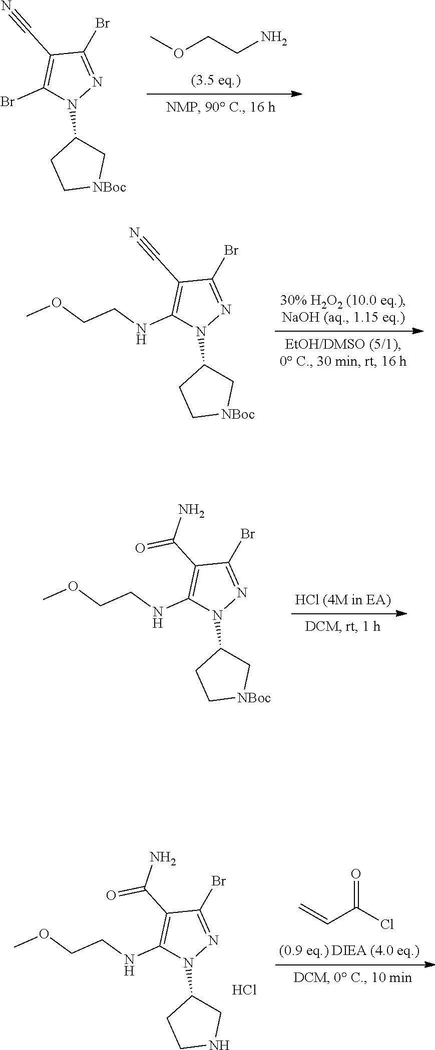

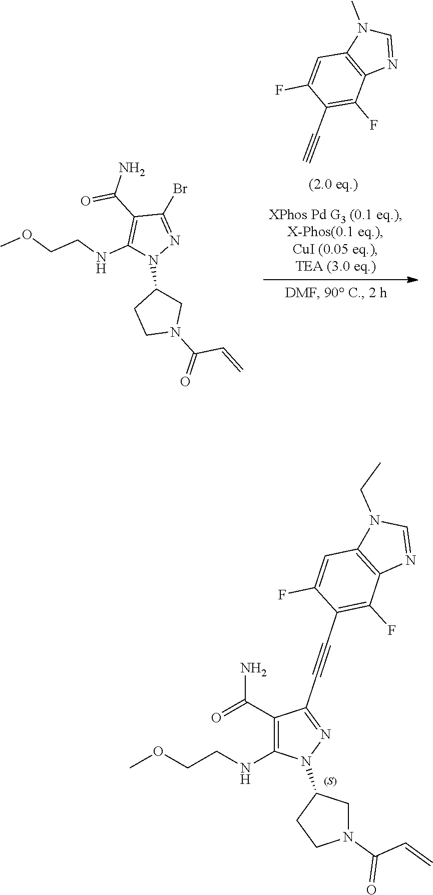

### Step 1: Tert-butyl (3S)-3-[3-bromo-4-cyano-5-[(2-methoxyethyl)amino]pyrazol-1-yl]pyrrolidine-1-carboxylate

To a stirred solution of tert-butyl (3S)-3-(3,5-dibromo-4-cyanopyrazol-1-yl)pyrrolidine-1-carboxylate (1.00 g, 2.38 mmol) in NMP (10.00 mL) was added 2-methoxyethan-1-amine (0.63 g, 8.38 mmol). The reaction mixture was stirred for 16 h at 90° C. The resulting mixture was cooled down, diluted with water (30 mL) and extracted with EtOAc (3×30 mL). The combined organic layers were washed with brine (3×30 mL), dried over anhydrous Na₂SO₄ and filtered. The filtrate was concentrated under reduced pressure to afford tert-butyl (3S)-3-[3-bromo-4-cyano-5-[(2-methoxyethyl)amino]pyrazol-1-yl]pyrrolidine-1-carboxylate (0.98 g, 99%) as a yellow oil which was used in the next step directly without further purification. MS ESI calculated for C₁₆H₂₄BrN₅O₃ [M+H−56]⁺, 358.11, 360.11, found 357.95, 359.95; ¹H NMR (300 MHz, CDCl₃) δ 6.41 (s, 1H), 4.99-4.95 (m, 1H), 4.78-4.05 (m, 2H), 3.86-3.57 (m, 5H), 3.43 (s, 3H), 2.62-1.98 (m, 2H), 1.29 (s, 9H).

### Step 2: Tert-butyl (3S)-3-[3-bromo-4-carbamoyl-5-[(2-methoxyethyl)amino]pyrazol-1-yl]pyrrolidine-1-carboxylate

To a stirred solution of tert-butyl (3S)-3-[3-bromo-4-cyano-5-[(2-methoxyethyl)amino]pyrazol-1-yl]pyrrolidine-1-carboxylate (0.98 g, 2.36 mmol) and NaOH (0.11 g, 2.72 mmol, 0.5 M) in EtOH (10.00 mL) and DMSO (2.00 mL) was added H₂O₂ (0.80 g, 23.65 mmol, 30%) dropwise at 0° C. The reaction mixture was stirred for 30 min at 0° C. and 16 h at room temperature. The resulting mixture was diluted with water (30 mL) and extracted with EtOAc (3×30 mL). The combined organic layers were washed with brine (3×30 mL), dried over anhydrous Na₂SO₄ and filtered. The filtrate was concentrated under reduced pressure. The residue was purified by silica gel Column chromatography, eluted with EA in PE (0-82%). The fractions contained desired product were combined and concentrated to afford tert-butyl (3S)-3-[3-bromo-4-carbamoyl-5-[(2-methoxyethyl)amino]pyrazol-1-yl]pyrrolidine-1-carboxylate (0.55 g, 53%) as an off-white solid. MS ESI calculated for C₁₆H₂₆BrN₅O₄ [M+H]⁺, 432.12, 434.12, found 432.15, 434.15.

### Step 3: 3-Bromo-5-[(2-methoxyethyl)amino]-1-[(3S)-pyrrolidin-3-yl]pyrazole-4-carboxamide hydrochloride

To a stirred solution of tert-butyl (3S)-3-[3-bromo-4-carbamoyl-5-[(2-methoxyethyl)amino]pyrazol-1-yl]pyrrolidine-1-carboxylate (0.55 g, 1.27 mmol) in DCM (6.00 mL) was added HCl (gas) in 1,4-dioxane (6.00 mL, 105.10 mmol) dropwise at 0° C. The reaction mixture was stirred for 1 h at room temperature. The resulting mixture was concentrated under reduced pressure to afford 3-bromo-5-[(2-methoxyethyl)amino]-1-[(3S)-pyrrolidin-3-yl]pyrazole-4-carboxamide hydrochloride (0.47 g, 99%) as an off-white solid which was used in the next step directly without further purification. MS ESI calculated for C₁₁H₁₈BrN₅O₂ [M+H]⁺, 332.07, 334.07, found 332.00, 334.00.

### Step 4: 3-Bromo-5-[(2-methoxyethyl)amino]-1-[(3S)-1-(prop-2-enoyl)pyrrolidin-3-yl]pyrazole-4-carboxamide

To a stirred solution of 3-bromo-5-[(2-methoxyethyl)amino]-1-[(3S)-pyrrolidin-3-yl]pyrazole-4-carboxamide hydrochloride (0.41 g, 1.11 mmol) in DCM (6.00 mL) were added DIEA (0.57 g, 4.44 mmol) and acryloyl chloride (4.00 mL, 1.00 mmol) dropwise at 0° C. under nitrogen atmosphere. The reaction mixture was diluted with water (30 mL) and extracted with CH₂Cl₂ (3×30 mL). The combined organic layers were washed with brine (30 mL), dried over anhydrous Na₂SO₄ and filtered. The filtrate was concentrated under reduced pressure. The residue was purified by silica gel Column chromatography, eluted with MeOH in DCM (0-4%). The fractions contained desired product were combined and concentrated to afford 3-bromo-5-[(2-methoxyethyl)amino]-1-[(3S)-1-(prop-2-enoyl)pyrrolidin-3-yl]pyrazole-4-carboxamide (0.37 g, 86%) as an off-white solid. MS ESI calculated for C₁₄H₂₀BrN₅O₃ [M+H]⁺, 386.08, 388.08, found 386.10, 388.10; ¹H NMR (300 MHz, CDCl₃) δ 6.61 (s, 1H), 6.54-6.32 (m, 2H), 5.79-5.69 (m, 1H), 5.51 (s, 1H), 5.21-4.93 (m, 1H), 4.13-3.84 (m, 3H), 3.79-3.50 (m, 3H), 3.48-3.15 (m, 5H), 2.68-2.48 (m, 1H), 2.45-2.25 (m, 1H).

### Step 5: 3-[2-(1-Ethyl-4,6-difluoro-1,3-benzodiazol-5-yl)ethynyl]-5-[(2-methoxyethyl)amino]-1-[(3S)-1-(prop-2-enoyl)pyrrolidin-3-yl]pyrazole-4-carboxamide

To a stirred solution of 3-bromo-5-[(2-methoxyethyl)amino]-1-[(3S)-1-(prop-2-enoyl)pyrrolidin-3-yl]pyrazole-4-carboxamide (0.15 g, 0.38 mmol) and 1-ethyl-5-ethynyl-4,6-difluoro-1,3-benzodiazole (0.16 g, 0.77 mmol) in DMF (2.50 mL) were added XPhos Pd G₃ (32.87 mg, 0.03 mmol), X-Phos (18.51 mg, 0.03 mmol), CuI (3.70 mg, 0.01 mmol) and TEA (0.11 g, 1.16 mmol) dropwise at room temperature. The reaction mixture was degassed with argon for three times and stirred for 2 h at 90° C. The resulting mixture was cooled down and concentrated under reduced pressure. The residue was purified by silica gel Column chromatography, eluted with MeOH in DCM (0-5%) to afford crude product. The crude product was purified by Prep-HPLC with the following conditions: Column: XBridge Prep Phenyl OBD Column, 19×150 mm 5 μm 13 nm; Mobile Phase A: Water (10 mmol/L NH₄HCO₃), Mobile Phase B: ACN; Flow rate: 20 mL/min; Gradient: 25 B to 50 B in 6 min, 210/254 nm; RT: 5.58 min. The fractions contained desired product were combined and concentrated to afford 3-[2-(1-ethyl-4,6-difluoro-1,3-benzodiazol-5-yl)ethynyl]-5-[(2-methoxyethyl)amino]-1-[(3S)-1-(prop-2-enoyl)pyrrolidin-3-yl]pyrazole-4-carboxamide (86.3 mg, 43%) as an off-white solid. MS ESI calculated for C₂₅H₂₇F₂N₇O₃[M+H]⁺, 512.21, found 512.15; ¹H NMR (300 MHz, CDCl₃) δ 7.97 (s, 1H), 7.23-6.97 (m, 2H), 6.65-6.26 (m, 2H), 5.86-5.66 (m, 1H), 5.47 (s, 1H), 5.27-4.98 (m, 1H), 4.23 (q, J=7.3 Hz, 2H), 4.16-3.86 (m, 3H), 3.84-3.65 (m, 1H), 3.65-3.53 (m, 2H), 3.48-3.24 (m, 5H), 2.77-2.51 (m, 1H), 2.40-2.35 (m, 1H), 1.58 (t, J=7.3 Hz, 3H).

### Example 120: 3-[2-(1-Ethyl-4,6-difluoro-1,3-benzodiazol-5-yl)ethynyl]-5-[[3-(morpholin-4-yl)propyl]amino]-1-[(3S)-1-(prop-2-enoyl)pyrrolidin-3-yl]pyrazole-4-carboxamide

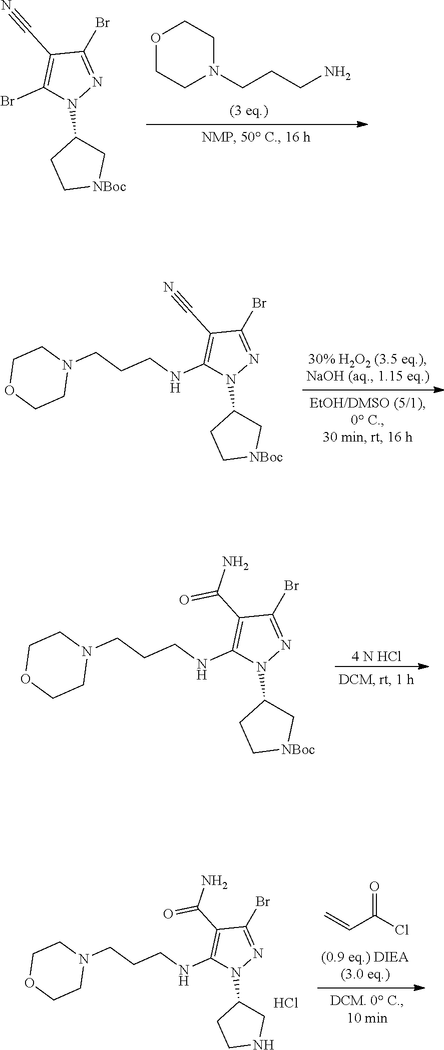

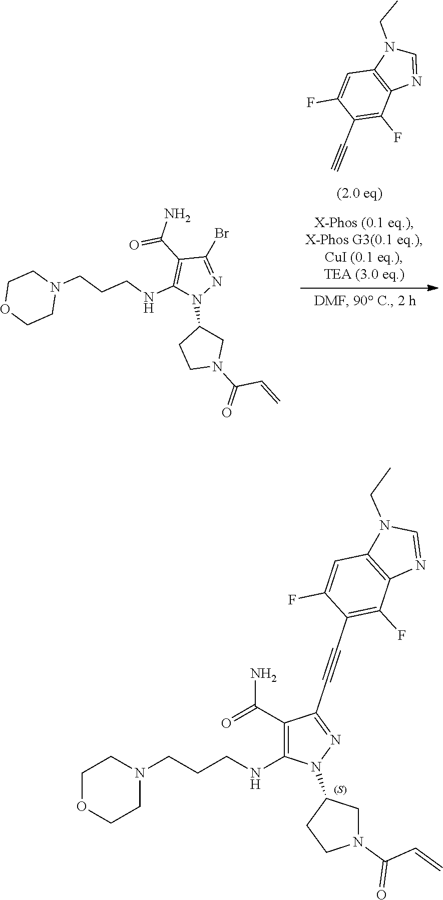

### Step 1: Tert-butyl (3S)-3-(3-bromo-4-cyano-5-[[3-(morpholin-4-yl)propyl]amino]pyrazol-1-yl)pyrrolidine-1-carboxylate

To a stirred mixture of tert-butyl (3S)-3-(3,5-dibromo-4-cyanopyrazol-1-yl)pyrrolidine-1-carboxylate (1.00 g, 2.38 mmol) in NMP (10.00 mL) was added 4-morpholinepropanamine (1.03 g, 7.14 mmol) dropwise at room temperature. The reaction mixture was stirred for 16 h at 50° C. under argon atmosphere. The resulting mixture was poured into water (100 mL) and extracted with EA (3×50 mL). The combined organic layers were washed with brine (2×100 mL), dried over anhydrous Na₂SO₄, filtrated and concentrated under reduced pressure to afford tert-butyl (3S)-3-(3-bromo-4-cyano-5-[[3-(morpholin-4-yl)propyl]amino]pyrazol-1-yl)pyrrolidine-1-carboxylate (1.0 g, 87%) as a light yellow oil which was used in the next step directly without further purification. MS ESI calculated for C₂₀H₃₁BrN₆O₃ [M+H]⁺, 483.16, found 483.30.

### Step 2: Tert-butyl (3S)-3-(3-bromo-4-carbamoyl-5-[[3-(morpholin-4-yl)propyl]amino]pyrazol-1-yl)pyrrolidine-1-carboxylate

To a stirred solution of tert-butyl (3S)-3-(3-bromo-4-cyano-5-[[3-(morpholin-4-yl)propyl]amino]pyrazol-1-yl)pyrrolidine-1-carboxylate (0.75 g, 1.55 mmol) and NaOH (0.5 M in water) (3.57 mL, 1.78 mmol) in DMSO (1.60 mL) and EtOH (8.00 mL) was added H₂O₂ (1.20 mL, 51.51 mmol, 30%) dropwise at 0° C. The reaction mixture was stirred for 30 min at 0° C. and 16 h at room temperature. The resulting mixture was diluted with water (100 mL) and extracted with EA (3×100 mL). The combined organic layers were washed with brine (100 mL), dried over anhydrous Na₂SO₄ and filtered. The filtrate was concentrated under reduced pressure. The residue was purified by silica gel Column chromatography, eluted with MeOH in DCM (0-5%). The fractions contained desired product were combined and concentrated to afford tert-butyl (3S)-3-(3-bromo-4-carbamoyl-5-[[3-(morpholin-4-yl)propyl]amino]pyrazol-1-yl)pyrrolidine-1-carboxylate (0.48 g, 62%) as an off-white oil. MS ESI calculated for C₂₀H₃₃BrN₆O₄ [M+H]⁺, 501.17, found 501.25.

### Step 3: 3-Bromo-5-[[3-(morpholin-4-yl)propyl]amino]-1-[(3S)-pyrrolidin-3-yl]pyrazole-4-carboxamide hydrochloride

To a stirred solution of tert-butyl (3S)-3-(3-bromo-4-carbamoyl-5-[[3-(morpholin-4-yl)propyl]amino]pyrazol-1-yl)pyrrolidine-1-carboxylate (0.48 mg, 0.96 mmol) in DCM (5.00 mL) was added HCl (4 M in EA) (5.00 mL) dropwise at 0° C. under nitrogen atmosphere. The reaction mixture was stirred for 1 h at room temperature. The resulting mixture was concentrated under reduced pressure to afford 3-bromo-5-[[3-(morpholin-4-yl)propyl]amino]-1-[(3S)-pyrrolidin-3-yl]pyrazole-4-carboxamide hydrochloride (0.4 g, 95%) as a yellow solid which was used in the next step directly without further purification. MS ESI calculated for C₁₅H₂₆BrClN₆O₂ [M+H]⁺, 401.12, found 401.15; ¹H NMR (400 MHz, CD₃OD) δ 5.49 (s, 1H), 5.37 (d, J=6.5 Hz, 2H), 4.09-4.05 (m, 1H), 3.88-3.72 (m, 4H), 3.74-3.71 (m, 2H), 3.68-3.63 (m, 1H), 3.62-3.59 (m, 2H), 3.49-3.45 (m, 1H), 3.42-3.33 (m, 4H), 3.17-3.15 (m, 2H), 2.66-2.55 (m, 1H), 2.34-2.26 (m, 1H), 2.10-2.08 (m, 2H), 2.02-2.19 (m, 1H).

### Step 4: 3-Bromo-5-[[3-(morpholin-4-yl)propyl]amino]-1-[(3S)-1-(prop-2-enoyl)pyrrolidin-3-yl]pyrazole-4-carboxamide

To a stirred solution of 3-bromo-5-[[3-(morpholin-4-yl)propyl]amino]-1-[(3S)-pyrrolidin-3-yl]pyrazole-4-carboxamide hydrochloride (0.40 g, 0.91 mmol) and DIEA (0.35 g, 2.741 mmol) in DCM (4.00 mL) was added acryloyl chloride (3.29 mL, 0.82 mmol, 0.25 M in DCM) dropwise at 0° C. The reaction mixture was stirred for 10 min at 0° C. under argon atmosphere. The resulting mixture was diluted with water (20 mL) and extracted with EA (3×20 mL). The combined organic layers were washed with brine (20 mL), dried over anhydrous Na₂SO₄ and filtered. The filtrate was concentrated under reduced pressure. The residue was purified by silica gel Column chromatography, eluted with MeOH in DCM (0-10%). The fractions contained desired product were combined and concentrated to afford 3-bromo-5-[[3-(morpholin-4-yl)propyl]amino]-1-[(3S)-1-(prop-2-enoyl)pyrrolidin-3-yl]pyrazole-4-carboxamide (0.2 g, 48%) as a white solid. MS ESI calculated for C₁₈H₂₇BrN₆O₃ [M+H]⁺, 457.13, found 457.15; ¹H NMR (400 MHz, CDCl₃) δ 6.64 (s, 2H), 6.43-6.35 (m, 1H), 5.76-5.66 (m, 1H), 5.45 (s, 1H), 4.99 (m, 1H), 4.04 (d, J=6.9 Hz, 1H), 4.03-3.98 (m, 1H), 3.98-3.95 (m, 1H), 3.96-3.85 (m, 1H), 3.85-3.81 (m, 4H), 3.69-3.66 (m, 3H), 1.93 (s, 3H), 1.56-1.46 (m, 4H), 1.26-1.23 (m, 2H), 0.90-0.81 (m, 1H).

### Step 5: 3-[2-(1-Ethyl-4,6-difluoro-1,3-benzodiazol-5-yl)ethynyl]-5-[[3-(morpholin-4-yl)propyl]amino]-1-[(3S)-1-(prop-2-enoyl)pyrrolidin-3-yl]pyrazole-4-carboxamide

To a stirred mixture of 3-bromo-5-[[3-(morpholin-4-yl)propyl]amino]-1-[(3S)-1-(prop-2-enoyl)pyrrolidin-3-yl]pyrazole-4-carboxamide (0.15 g, 0.33 mmol), 1-ethyl-5-ethynyl-4,6-difluoro-1,3-benzodiazole (0.14 g, 0.66 mmol), X-Phos (15.70 mg, 0.03 mmol), XPhos Pd G3 (27.88 mg, 0.03 mmol) and CuI (6.27 mg, 0.03 mmol) in DMF (4.00 mL) was added TEA (0.10 g, 0.99 mmol) dropwise at room temperature. The reaction mixture was degassed with argon for three times and stirred for 2 h at 90° C. The resulting mixture was cooled down and concentrated under reduced pressure. The residue was purified by silica gel Column chromatography, eluted with MeOH in DCM (0-10%) to afford the crude product. Then the crude product was purified by Prep-HPLC with the following conditions: Column: X-Bridge Prep C18 OBD Column, 19×150 mm 5 μm; Mobile Phase A: Water (10 mmol/L NH₄HCO₃), Mobile Phase B: ACN; Flow rate: 20 mL/min; Gradient: 15 B to 50 B in 5.8 min, 210/254 nm. The fractions contained desired product were combined and concentrated to afford 3-[2-(1-ethyl-4,6-difluoro-1,3-benzodiazol-5-yl)ethynyl]-5-[[3-(morpholin-4-yl)propyl]amino]-1-[(3S)-1-(prop-2-enoyl)pyrrolidin-3-yl]pyrazole-4-carboxamide (30.2 mg, 16%) as an off-white solid. MS ESI calculated for C₂₉H₃₄F₂N₈O₃[M+H]⁺, 581.27, found 581.30; ¹H NMR (300 MHz, CDCl₃) δ 7.94 (s, 1H), 7.20-6.93 (m, 2H), 6.58-6.31 (m, 3H), 5.83-5.65 (m, 1H), 5.41 (s, 1H), 5.05-5.00 (m, 1H), 4.26-4.21 (m, 2H), 4.12-3.87 (m, 3H), 3.72-3.69 (m, 5H), 3.26-3.24 (m, 2H), 2.78-2.57 (m, 1H), 2.46-2.44 (m, 7H), 1.85-1.75 (m, 2H), 1.66 (s, 3H).

### Example 121: 1-((3S,5R)-1-Acryloyl-5-(methoxymethyl)pyrrolidin-3-yl)-3-((7-fluoro-1-methyl-1H-benzo[d]imidazol-5-yl)ethynyl)-5-(methylamino)-1H-pyrazole-4-carboxamide

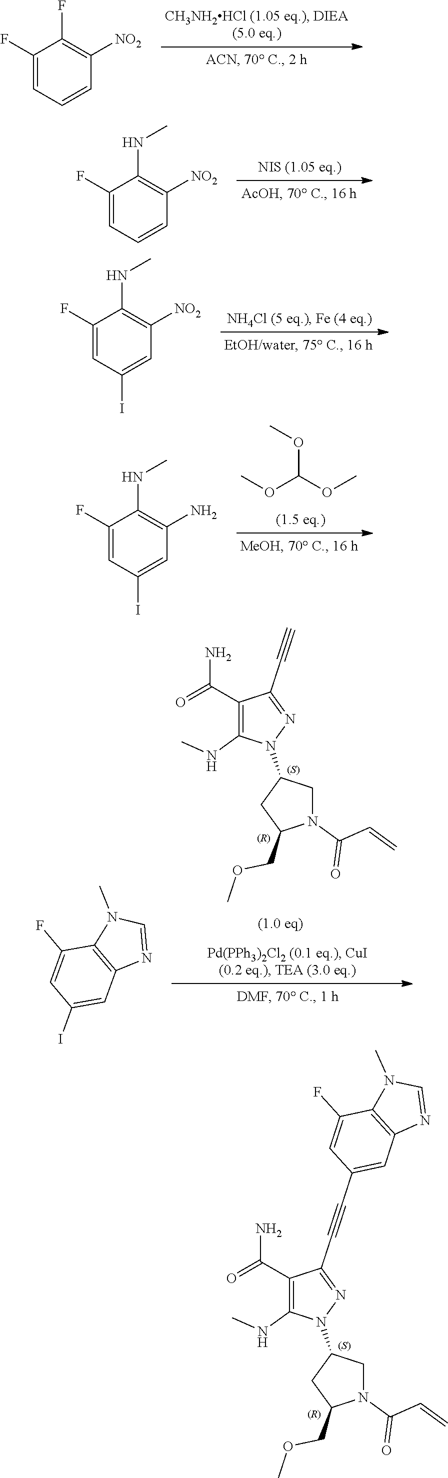

### Step 1: 2-Fluoro-N-methyl-6-nitroaniline

To a stirred mixture of 1,2-difluoro-3-nitrobenzene (1.00 g, 6.28 mmol) in ACN (9.00 mL) were added CH₃NH₂HCl (0.45 g, 6.66 mmol) and DIEA (5.47 mL, 42.35 mmol). The reaction mixture was stirred for 2 h at 70° C. The resulting mixture was cooled down and concentrated under reduced pressure. The residue was diluted with water (20 mL) and extracted with EA (3×20 mL). The combined organic layers were washed with brine (3×20 mL), dried over anhydrous Na₂SO₄ and filtered. The filtrate was concentrated under reduced pressure to afford 2-fluoro-N-methyl-6-nitroaniline (0.98 g, crude) as a yellow solid which was used in the next step directly without further purification. ¹H NMR (300 MHz, CDCl₃) δ 7.94-7.83 (m, 2H), 7.21-7.14 (m, 1H), 6.59-6.52 (m, 1H), 3.27-3.23 (m, 3H).

### Step 2: 2-Fluoro-4-iodo-N-methyl-6-nitroaniline

To a stirred solution of 2-fluoro-N-methyl-6-nitroaniline (0.96 g, 5.6 mmol) in AcOH (9.60 mL) was added NIS (1.33 g, 5.92 mmol) at 70° C. The reaction mixture was stirred for 16 h at 70° C. under nitrogen atmosphere. The resulting mixture was concentrated under reduced pressure. The residue was diluted with water (100 mL), adjusted to pH 8 with NaHCO₃ and extracted with EtOAc (3×100 mL). The combined organic layers were washed with brine (3×100 mL), dried over anhydrous Na₂SO₄ and filtered. The filtrate was concentrated under reduced pressure. The residue was purified by silica gel Column chromatography, eluted with 17% DCM in PE. The fractions contained desired product were combined and concentrated to afford 2-fluoro-4-iodo-N-methyl-6-nitroaniline (1.48 g, 88%) as a yellow solid. ¹H NMR (300 MHz, CDCl₃) δ 8.26 (s, 1H), 7.87 (s, 1H), 7.44-7.39 (m, 1H), 3.25-3.23 (m, 3H)

### Step 3: 6-Fluoro-4-iodo-N1-methylbenzene-1,2-diamine

To a stirred mixture of 2-fluoro-4-iodo-N-methyl-6-nitroaniline (0.80 g, 2.70 mmol) and Fe (0.60 g, 10.80 mmol) in EtOH (14.00 mL) and water (2.00 mL) was added NH₄Cl (0.72 g, 13.51 mmol). The reaction mixture was stirred for 16 h at 75° C. The resulting mixture was cooled and filtered, the filter cake was washed with EtOH (3×50 mL). The filtrate was concentrated under reduced pressure. The residue was diluted with water (50 mL) and extracted with EA (3×50 mL). The combined organic layers were washed with brine (3×50 mL), dried over anhydrous Na₂SO₄ and filtered. The filtrate was concentrated under reduced pressure to afford 6-fluoro-4-iodo-N¹-methylbenzene-1,2-diamine (0.69 g, crude) as a brown solid which was used in the next step directly without further purification. MS ESI calculated for C₇H₈FIN₂ [M+H]⁺, 266.98, found 266.95.

### Step 4: 7-Fluoro-5-iodo-1-methyl-1,3-benzodiazole

To a stirred mixture of 6-fluoro-4-iodo-N¹-methylbenzene-1,2-diamine (0.69 g, 2.60 mmol) in MeOH (7.00 mL) was added trimethyl orthoformate (0.41 g, 3.90 mmol). The reaction mixture was stirred for 16 h at 70° C. The resulting mixture was concentrated under reduced pressure. The residue was purified by silica gel Column chromatography, eluted with 8% MeOH in DCM. The fractions contained desired product were combined and concentrated to afford 7-fluoro-5-iodo-1-methyl-1,3-benzodiazole (0.20 g, 87%) as a light brown solid. MS ESI calculated for C₈H₆FIN₂ [M+H]⁺, 276.96, found 277.00; ¹H NMR (300 MHz, CDCl₃) δ 7.92 (d, J=1.3 Hz, 1H), 7.73 (s, 1H), 7.34-7.21 (m, 1H), 4.00 (s, 3H).

### Step 5: 1-((3S,5R)-1-Acryloyl-5-(methoxymethyl)pyrrolidin-3-yl)-3-((7-fluoro-1-methyl-1H-benzo[d]imidazol-5-yl)ethynyl)-5-(methylamino)-1H-pyrazole-4-carboxamide

To a stirred mixture of 7-fluoro-5-iodo-1-methyl-1,3-benzodiazole (0.12 g, 0.45 mmol), 3-ethynyl-1-[(3S,5R)-5-(methoxymethyl)-1-(prop-2-enoyl) pyrrolidin-3-yl]-5-(methylamino) pyrazole-4-carboxamide (0.15 g, 0.45 mmol), CuI (17.24 mg, 0.09 mmol) and Pd(PPh₃)₂Cl₂ (31.77 mg, 0.04 mmol) in DMF (1.2 mL) was added TEA (0.19 mL, 1.86 mmol) at room temperature. The reaction mixture was degassed with argon for three times and stirred for 1 h at 70° C. The resulting mixture was concentrated under reduced pressure. The residue was purified by silica gel Column chromatography, eluted with 8% MeOH in DCM. The fractions contained desired product were combined and concentrated. The crude product was purified by Prep-HPLC with the following conditions: Column: XBridge Prep C18 OBD Column, 19×150 mm 5 μm; Mobile Phase A: Water (10 mmol/L NH₄HCO₃), Mobile Phase B: ACN; Flow rate: 20 mL/min; Gradient: 20 B to 50 B in 5.8 min, 210/254 nm. The fractions contained desired product were combined and concentrated to afford 1-((3S,5R)-1-acryloyl-5-(methoxymethyl)pyrrolidin-3-yl)-3-((7-fluoro-1-methyl-1H-benzo[d]imidazol-5-yl)ethynyl)-5-(methylamino)-1H-pyrazole-4-carboxamide (79.6 mg, 36%) as an off-white solid. MS ESI calculated for C₂₄H₂₆FN₇O₃[M+H]⁺, 480.21, found 480.30; ¹H NMR (300 MHz, DMSO-d₆) δ 8.36 (s, 1H), 7.75 (d, J=1.2 Hz, 1H), 7.39-7.18 (m, 2H), 6.65-6.61 (m, 2H), 6.21-6.11 (m, 1H), 5.68-5.65 (m, 1H), 5.32-5.15 (m, 1H), 4.46-4.32 (m, 1H), 4.00-3.97 (m, 3H), 3.92-3.46 (m, 4H), 3.29 (m, 3H), 2.94 (d, J=3.1 Hz, 3H), 2.43-2.41 (m, 1H), 2.31-2.27 (m, 1H).

### Example 122: 3-(2-[7-Fluoro-[1,2,4]triazolo[1,5-a]pyridin-6-yl]ethynyl)-1-[(3S,5R)-5-(methoxymethyl)-1-(prop-2-enoyl)pyrrolidin-3-yl]-5-(methylamino)pyrazole-4-carboxamide

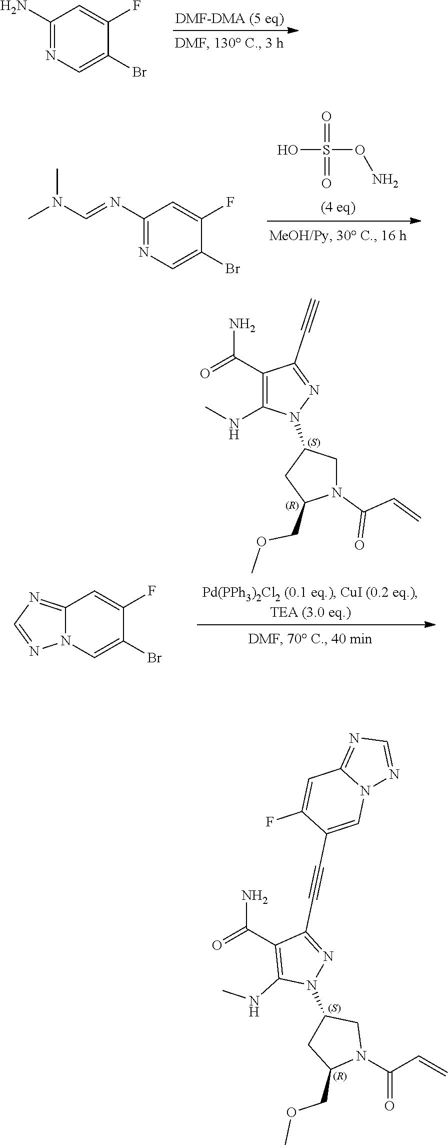

### Step 1: (E)-N¹-(5-Bromo-4-fluoropyridin-2-yl)-N,N-dimethylmethanimidamide

To a stirred solution of 5-bromo-4-fluoropyridin-2-amine (3.00 g, 15.70 mmol) in DMF (10.00 mL) was added DMF-DMA (9.36 g, 78.53 mmol) at room temperature. The reaction mixture was stirred for 3 h at 130° C. under argon atmosphere. The resulting mixture was concentrated under reduced pressure. The residue was purified by silica gel column chromatography, eluted with PE/EA (2/1). The fractions contained desired product were combined and concentrated to afford (E)-N′-(5-bromo-4-fluoropyridin-2-yl)-N,N-dimethylmethanimidamide (3 g, 77%) as a white solid. MS ESI calculated for C₈H₉BrFN₃ [M+H]⁺, 246.00, found 246.05; ¹H NMR (400 MHz, CDCl₃) δ 8.44 (s, 1H), 8.27 (d, J=9.7 Hz, 1H), 6.70 (d, J=10.1 Hz, 1H), 3.11-2.95 (m, 6H).

### Step 2: 6-Bromo-7-fluoro-[1,2,4]triazolo[1,5-a]pyridine

To a stirred solution of (E)-N′-(5-bromo-4-fluoropyridin-2-yl)-N,N-dimethyl methanimidamide (3.00 g, 12.19 mmol) in MeOH (60.00 mL) and Pyridine (4.82 g, 60.95 mmol) was added aminooxysulfonic acid (5.51 g, 48.76 mmol) at 0° C. The reaction mixture was stirred for 16 h at 30° C. under nitrogen atmosphere. The resulting mixture was concentrated under reduced pressure. The residue was purified by silica gel column chromatography, eluted with PE/EA (2/1). The fractions contained desired product were combined and concentrated to afford 6-bromo-7-fluoro-[1,2,4]triazolo[1,5-a]pyridine (0.47 g, 17%) as a white solid. MS ESI calculated for C₆H₃BrFN₃ [M+H]⁺, 215.95, 217.95, found 216.00, 218.00; ¹H NMR (400 MHz, DMSO-d₆) δ 9.61-9.23 (m, 1H), 8.55 (s, 1H), 8.02 (d, J=8.9 Hz, 1H).

### Step 3: 3-(2-[7-Fluoro-[1,2,4]triazolo[1,5-a]pyridin-6-yl]ethynyl)-1-[(3S,5R)-5-(methoxymethyl)-1-(prop-2-enoyl)pyrrolidin-3-yl]-5-(methylamino)pyrazole-4-carboxamide

To a stirred solution of 3-ethynyl-1-[(3S,5R)-5-(methoxymethyl)-1-(prop-2-enoyl)pyrrolidin-3-yl]-5-(methylamino)pyrazole-4-carboxamide (0.2 g, 0.60 mmol), 6-bromo-7-fluoro-[1,2,4]triazolo[1,5-a]pyridine (0.13 g, 0.60 mmol), Pd(PPh₃)₂Cl₂ (42.36 mg, 0.06 mmol) and CuI (22.99 mg, 0.12 mmol) in DMF (2.00 mL) was added TEA (0.18 g, 1.81 mmol) dropwise at room temperature. The reaction mixture was degassed with argon for three times and stirred for 40 min at 70° C. The resulting mixture was concentrated under reduced pressure. The residue was purified by silica gel column chromatography, eluted with CH₂Cl₂/MeOH (10/1). The fractions contained desired product were combined and concentrated. The crude product was purified by Prep-HPLC with the following conditions: Column: XBridge Prep C18 OBD Column, 19×150 mm 5 μm; Mobile Phase A: Water (10 mmol/L NH₄HCO₃), Mobile Phase B: ACN; Flow rate: 20 mL/min; Gradient: 35 B to 55 B in 6 min; 210/254 nm. The fractions contained desired product were combined and concentrated to afford 3-(2-[7-fluoro-[1,2,4]triazolo[1,5-a]pyridin-6-yl]ethynyl)-1-[(3S,5R)-5-(methoxymethyl)-1-(prop-2-enoyl)pyrrolidin-3-yl]-5-(methylamino)pyrazole-4-carboxamide (89.4 mg, 31%) as a white solid. MS ESI calculated for C₂₂H₂₃FN₃O₃[M+H]⁺, 467.20, found 467.20; ¹H NMR (400 MHz, CDCl₃) δ 8.87 (d, J=6.1 Hz, 1H), 8.41 (s, 1H), 7.51 (d, J=8.8 Hz, 1H), 7.12-6.52 (m, 1H), 6.50-6.35 (m, 2H), 5.85-5.65 (m, 1H), 5.61-5.25 (m, 2H), 4.65-4.34 (m, 1H), 4.23-3.85 (m, 3H), 3.55-3.42 (m, 1H), 3.39 (d, J=5.5 Hz, 3H), 3.06 (d, J=14.2 Hz, 3H), 2.96-2.61 (m, 1H), 2.38-2.13 (m, 1H).

### Example 123: 3-[2-(1-Ethyl-4,6-difluoro-1,3-benzodiazol-5-yl)ethynyl]-5-[(2-hydroxyethyl)amino]-1-[(3S)-1-(prop-2-enoyl)pyrrolidin-3-yl]pyrazole-4-carboxamide

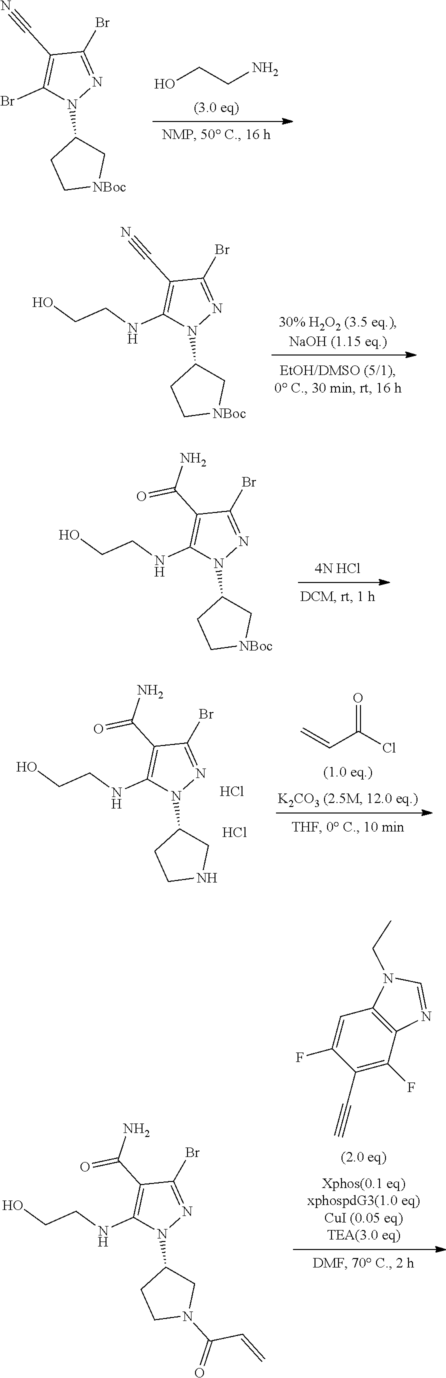

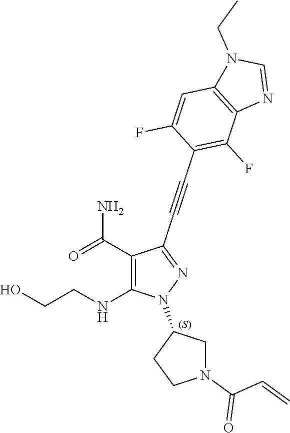

### Step 1: Tert-butyl (3S)-3-[3-bromo-4-cyano-5-[(2-hydroxyethyl)amino]pyrazol-1-yl]pyrrolidine-1-carboxylate

To a stirred solution of tert-butyl (3S)-3-(3,5-dibromo-4-cyanopyrazol-1-yl) pyrrolidine-1-carboxylate (3.00 g, 7.14 mmol) in NMP (30.00 mL) was added ethanolamine (1.32 g, 21.64 mmol) dropwise. The reaction mixture was stirred for 16 h at 50° C. under argon atmosphere. The resulting mixture was cooled down and concentrated under reduced pressure. The residue was diluted with water (200 mL) and extracted with EA (3×200 mL). The combined organic layers were washed with brine (400 mL), dried over anhydrous Na₂SO₄ and filtered. The filtrate was concentrated under reduced pressure to afford tert-butyl (3S)-3-[3-bromo-4-cyano-5-[(2-hydroxyethyl)amino]pyrazol-1-yl]pyrrolidine-1-carboxylate (3.03 g, crude) as a light yellow oil which was used in the next step directly without further purification. MS ESI calculated for C₁₅H₂₂BrN₅O₃ [M+H]⁺, 400.09, found 400.10; ¹H-NMR (300 MHz, CDCl₃) δ 6.44 (s, 1H), 5.03-4.99 (m, 2H), 4.31-3.39 (m, 5H), 2.66-2.20 (m, 2H), 2.07 (s, 1H), 1.48 (s, 2H) 1.28 (d, J=6.2 Hz, 9H).

### Step 2: Tert-butyl (3S)-3-[3-bromo-4-carbamoyl-5-[(2-hydroxyethyl)amino]pyrazol-1-yl]pyrrolidine-1-carboxylate

To a stirred solution of tert-butyl (3S)-3-[3-bromo-4-cyano-5-[(2-hydroxyethyl)amino]pyrazol-1-yl]pyrrolidine-1-carboxylate (1.50 g, 3.75 mmol) in EtOH (15.00 mL) and DMSO (3.00 mL) were added NaOH (8.61 mL, 4.31 mmol, 0.5 M) and H₂O₂ (0.90 mL, 13.31 mmol, 30%) at 0° C. The reaction mixture was stirred for 30 min at 0° C. and 16 h at room temperature. The resulting mixture was diluted with water (100 mL) and extracted with DCM (3×100 mL). The combined organic layers were washed with brine (2×100 mL), dried over anhydrous Na₂SO₄ and filtered. The filtrate was concentrated under reduced pressure. The residue was purified by silica gel column chromatography, eluted with PE/(EA:EtOH (3:1)). The fractions contained desired product were combined and concentrated to afford tert-butyl (3S)-3-[3-bromo-4-carbamoyl-5-[(2-hydroxyethyl)amino]pyrazol-1-yl]pyrrolidine-1-carboxylate (0.58 g, 37%) as a light yellow solid. MS ESI calculated for C₁₅H₂₄BrN₅O₄ [M+H]⁺, 416.10, found 416.15; ¹H-NMR (300 MHz, CDCl₃) δ 6.77 (s, 1H), 5.68 (s, 1H), 4.90 (t, J=7.4 Hz, 1H), 4.18-3.59 (m, 5H), 3.52-3.18 (m, 3H), 2.55-2.16 (m, 2H), 2.06 (d, J=10.9 Hz, 1H), 1.47 (s, 9H), 1.26 (t, J=7.2 Hz, 1H).

### Step 3: 3-Bromo-5-[(2-hydroxyethyl)amino]-1-[(3S)-pyrrolidin-3-yl]pyrazole-4-carboxamidedihydrochloride

To a stirred solution of tert-butyl (3S)-3-[3-bromo-4-carbamoyl-5-[(2-hydroxyethyl)amino]pyrazol-1-yl]pyrrolidine-1-carboxylate (0.58 g, 1.38 mmol) in DCM (10 mL) was added HCl (6 mL, 4 M in EA) at 0° C. The reaction mixture was stirred for 1 h at rt. The resulting mixture was concentrated under reduced pressure to afford 3-bromo-5-[(2-hydroxyethyl)amino]-1-[(3S)-pyrrolidin-3-yl]pyrazole-4-carboxamide dihydrochloride (0.72 g, crude) which was used in the next step directly without further purification. MS ESI calculated for C₁₀H₁₆BrN₅O₂ [M+H]⁺, 318.18, found 318.15.

### Step 4: 3-Bromo-5-[(2-hydroxyethyl)amino]-1-[(3S)-1-(prop-2-enoyl)pyrrolidin-3-yl]pyrazole-4-carboxamide (EB2000269-024)

To a stirred mixture of 3-bromo-5-[(2-hydroxyethyl)amino]-1-[(3S)-pyrrolidin-3-yl]pyrazole-4-carboxamide dihydrochloride (0.54 g, 1.38 mmol) and K₂CO₃ (2.28 g, 16.54 mmol) in THF (5.50 mL) was added acryloyl chloride (6.66 mL, 16.66 mmol, 2.5 M) in THF (10.00 mL) dropwise at 0° C. under argon atmosphere. The reaction mixture was stirred for 10 min at 0° C. under argon atmosphere. The resulting mixture was diluted with water (80 mL) and extracted with DCM (3×80 mL). The combined organic layers were washed with brine (2×50 mL), dried over anhydrous Na₂SO₄ and filtered. The filtrate was concentrated under reduced pressure. The residue was purified by silica gel column chromatography, eluted with 8% MeOH in DCM. The fractions contained desired product were combined and concentrated to afford 3-bromo-5-[(2-hydroxyethyl)amino]-1-[(3S)-1-(prop-2-enoyl)pyrrolidin-3-yl]pyrazole-4-carboxamide (0.32 g, 62%) as a white solid. MS ESI calculated for C₁₃H₁₈BrN₅O₃ [M+H]⁺, 372.22, found 372.10; ¹H-NMR (300 MHz, CDCl₃) δ 6.78 (s, 1H), 6.58-6.37 (m, 2H), 5.81-5.58 (m, 2H), 5.04 (dd, J=14.7, 7.3 Hz, 1H), 4.13-3.56 (m, 7H), 3.29 (d, J=4.7 Hz, 2H), 2.72-2.34 (m, 3H).

### Step 5: 3-[2-(1-Ethyl-4,6-difluoro-1,3-benzodiazol-5-yl)ethynyl]-5-[(2-hydroxyethyl)amino]-1-[(3S)-1-(prop-2-enoyl)pyrrolidin-3-yl]pyrazole-4-carboxamide

To a stirred mixture of 3-bromo-5-[(2-hydroxyethyl) amino]-1-[(3S)-1-(prop-2-enoyl) pyrrolidin-3-yl]pyrazole-4-carboxamide (0.18 g, 0.47 mmol), 1-ethyl-5-ethynyl-4,6-difluoro-1,3-benzodiazole (0.19 g, 0.94 mmol), CuI (4.48 mg, 0.02 mmol), X-Phos (22.41 mg, 0.05 mmol) and XPhos Pd G3 (39.80 mg, 0.05 mmol) in DMF (1.75 mL) was added TEA (0.20 mL, 1.94 mmol). The reaction mixture was degassed with argon for three times and stirred for 2 h at 70° C. The resulting mixture was concentrated under reduced pressure. The residue was purified by silica gel column chromatography, eluted with 8% MeOH in DCM. The fractions contained desired product were combined and concentrated. The crude product was purified by Prep-HPLC with the following conditions: Column: X Bridge Prep C18 OBD Column, 19×150 mm 5 μm; Mobile Phase A: Water (10 mmol/L NH₄HCO₃), Mobile Phase B: ACN; Flow rate: 20 mL/min; Gradient: 35 B to 70 B in 5.8 min, 210/254 nm; RT: 5.58 min. The fractions contained desired product were combined and concentrated to afford 3-[2-(1-ethyl-4,6-difluoro-1,3-benzodiazol-5-yl)ethynyl]-5-[(2-hydroxyethyl)amino]-1-[(3S)-1-(prop-2-enoyl)pyrrolidin-3-yl]pyrazole-4-carboxamide (79.6 mg, 34%) as a white solid. MS ESI calculated for C₂₄H₂₅F₂N₇O₃[M+H]⁺ 498.20, found 498.25; ¹H-NMR (400 MHz, DMSO-d₆) δ 8.45 (s, 1H), 7.76-7.42 (m, 2H), 6.87-6.52 (m, 3H), 6.17-6.15 (m, 1H), 5.70-5.68 (m, 1H), 5.28-5.07 (m, 1H), 4.87-4.82 (m, 1H), 4.30 (q, J=7.3 Hz, 2H), 3.86-3.81 (m, 2H), 3.79-3.63 (m, 2H), 3.56-3.51 (m, 2H), 3.33-3.27 (m, 2H), 2.36-2.31 (m, 2H), 1.41 (t, J=7.2 Hz, 3H).

### Example 124: 3-[2-(6-Chloro-1-cyclopropyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

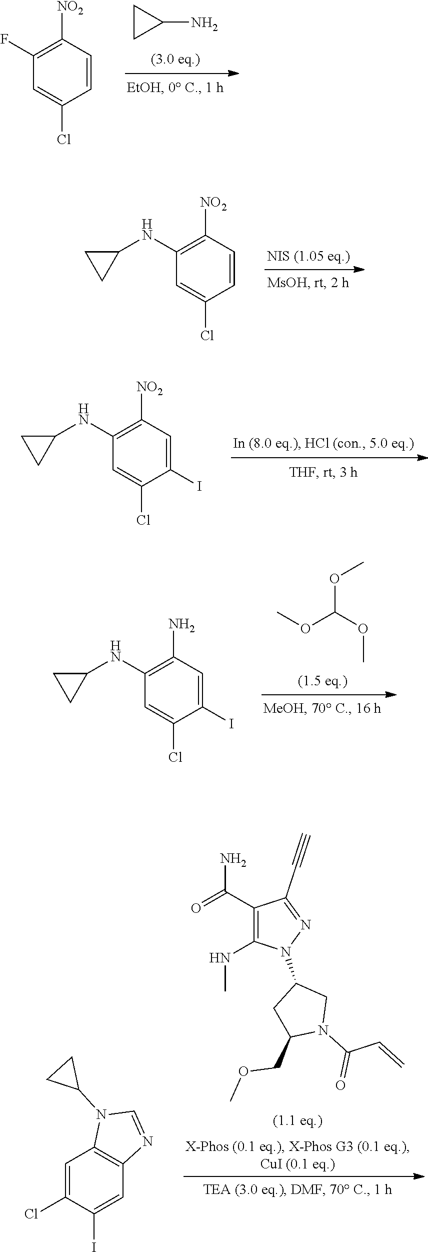

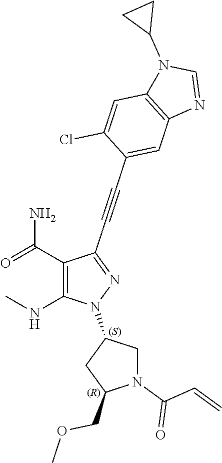

### Step 1: 5-Chloro-N-cyclopropyl-2-nitroaniline

To a stirred solution of 4-chloro-2-fluoro-1-nitrobenzene (10.00 g, 56.97 mmol) in EtOH (100.00 mL) was added aminocyclopropane (9.76 g, 170.90 mmol) dropwise at 0° C. The resulting mixture was stirred for 1 h at 0° C. The resulting mixture was filtered, the filter cake was washed with water (3×100 mL) and dried to afford 5-chloro-N-cyclopropyl-2-nitroaniline (12 g, 99%) as a yellow solid which was used in the next step directly without further purification. MS ESI calculated for C₉H₉ClN₂O₂[M+H]⁺, 213.04, 215.04, found 213.10, 215.10; ¹H NMR (400 MHz, CDCl₃) δ 8.13 (s, 1H), 8.12 (d, J=9.1 Hz, 1H), 7.32 (d, J=2.2 Hz, 1H), 6.68 (dd, J=9.1, 2.2 Hz, 1H), 2.62-2.56 (m, 1H), 1.04-0.93 (m, 2H), 0.74-0.66 (m, 2H).

### Step 2: 5-Chloro-N-cyclopropyl-4-iodo-2-nitroaniline

To a stirred mixture of 5-chloro-N-cyclopropyl-2-nitroaniline (12.00 g, 56.44 mmol) in methanesulfonic acid (60 mL) was added NIS (13.33 g, 59.26 mmol) in portions at 0° C. The reaction mixture was stirred for 2 h at room temperature. The resulting mixture was quenched with ice/water (100 mL) at 0° C., adjusted to pH 8 with NaOH and extracted with EtOAc (3×100 mL). The combined organic layers were washed with brine (3×100 mL), dried over anhydrous Na₂SO₄ and filtered. The filtrate was concentrated under reduced pressure. The residue was purified by silica gel Column chromatography, eluted with PE/EA. The fractions contained desired product were combined and concentrated to afford 5-chloro-N-cyclopropyl-4-iodo-2-nitroaniline (10 g, 52%) as an orange solid. MS ESI calculated for C₉H₈ClN₂O₂[M−H]⁻, 336.93, 338.93, found 336.90, 338.85; ¹H NMR (300 MHz, CDCl₃) δ 8.60 (s, 1H), 8.03 (s, 1H), 7.47 (s, 1H), 2.62-2.51 (m, 1H), 1.07-0.87 (m, 2H), 0.81-0.64 (m, 2H).

### Step 3: 5-Chloro-N1-cyclopropyl-4-iodobenzene-1,2-diamine

To a stirred mixture of 5-chloro-N-cyclopropyl-4-iodo-2-nitroaniline (9.00 g, 26.59 mmol) and In (24.42 g, 212.68 mmol) in THF (60.00 mL) was added con. HCl (11.08 mL, 132.96 mmol) dropwise at 0° C. The reaction mixture was stirred for 3 h at room temperature under nitrogen atmosphere. The resulting mixture was concentrated under reduced pressure. The residue was purified by silica gel Column chromatography, eluted with PE/EtOAc (1/1). The fractions contained desired product were combined and concentrated to afford 5-chloro-N¹-cyclopropyl-4-iodobenzene-1,2-diamine (3 g, 36%) as a light brown solid. MS ESI calculated for C₉H₁₀ClIN₂ [M+H]⁺, 308.96, 310.96, found 308.95, 310.95.

### Step 4: 6-Chloro-1-cyclopropyl-5-iodo-1,3-benzodiazole

To a stirred solution of 5-chloro-N¹-cyclopropyl-4-iodobenzene-1,2-diamine (3.00 g, 9.72 mmol) in MeOH (30.00 mL) was added trimethyl orthoformate (1.55 g, 14.58 mmol). The reaction mixture was stirred for 16 h at 70° C. The resulting mixture was cooled down and concentrated under reduced pressure. The residue was purified by silica gel Column chromatography, eluted with PE/EtOAc (1/1). The fractions contained desired product were combined and concentrated to afford 6-chloro-1-cyclopropyl-5-iodo-1,3-benzodiazole (0.90 g, 29%) as a light brown solid. MS ESI calculated for C₁₀H₈ClN₂ [M+H]⁺, 318.95, 320.95, found 318.95, 320.95; ¹H NMR (300 MHz, CDCl₃) δ 8.30 (s, 1H), 7.92 (s, 1H), 7.74 (s, 1H), 3.39-3.33 (m, 1H), 1.24-1.18 (m, 2H), 1.10-1.01 (m, 2H).

### Step 5: 3-[2-(6-Chloro-1-cyclopropyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a stirred mixture of 6-chloro-1-cyclopropyl-5-iodo-1,3-benzodiazole (50.00 mg, 0.16 mmol) and 3-ethynyl-1-[(3S,5R)-5-(methoxymethyl)-1-(prop-2-enoyl)pyrrolidin-3-yl]-5-(methylamino)pyrazole-4-carboxamide (57.22 mg, 0.17 mmol) in DMF (1.50 mL) were added XPhos Pd G3 (13.29 mg, 0.02 mmol), XPhos Pd G3 (13.29 mg, 0.02 mmol), CuI (2.99 mg, 0.02 mmol) and TEA (47.65 mg, 0.47 mmol). The reaction mixture was degassed with argon for three times and stirred for 1 h at 70° C. The resulting mixture was cooled down and concentrated under reduced pressure. The residue was purified by silica gel Column chromatography, eluted with CH₂Cl₂/MeOH (10/1) to afford crude. The crude product was purified by Prep-HPLC with the following conditions: Column: SunFire Prep C18 OBD Column, 19×150 mm 5 μm 10 nm; Mobile Phase A: Water (10 mmol/L, NH₄HCO₃), Mobile Phase B: ACN; Flow rate: 20 mL/min; Gradient: 25 B to 50 B in 6 min; 210/254 nm; RT: 5.58 min. The fractions contained desired product were combined and concentrated to afford 3-[2-(6-chloro-1-cyclopropyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (41.5 mg, 50%) as an off-white solid. MS ESI calculated for C₂₆H₂₈ClN₇O₃[M+H]⁺, 522.19, found 522.25; ¹H NMR (400 MHz, CDCl₃) δ 8.07 (d, J=5.9 Hz, 2H), 7.68 (s, 1H), 7.20 (s, 1H), 6.82 (s, 1H), 6.59-6.36 (m, 2H), 5.73-5.70 (m, 1H), 5.57-5.40 (m, 2H), 4.59-4.45 (m, 1H), 4.17-3.95 (m, 2H), 3.93-3.89 (m, 1H), 3.50-3.36 (m, 5H), 3.06 (s, 3H), 2.79-2.67 (m, 1H), 2.34-2.31 (m, 1H), 1.28-1.17 (m, 2H), 1.19-1.05 (m, 2H).

### Example 125: 3-[2-(1-Ethyl-6,7-difluoro-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

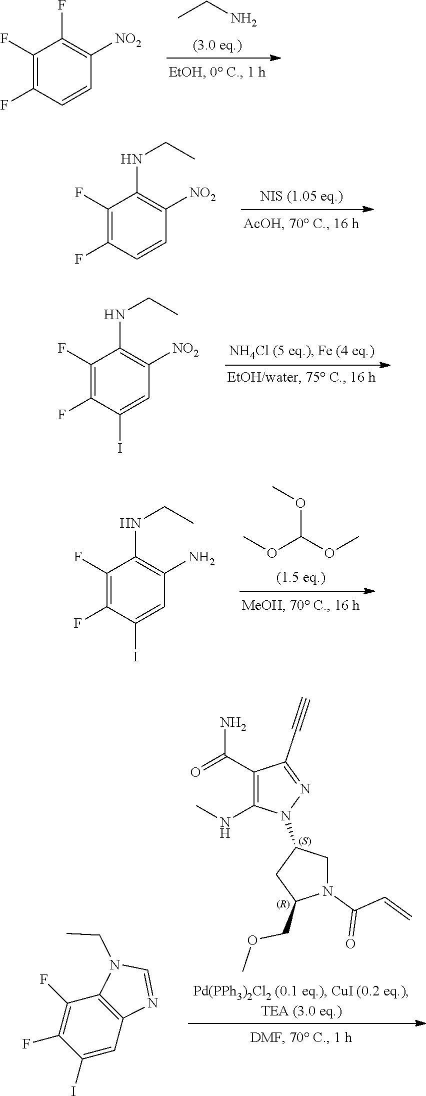

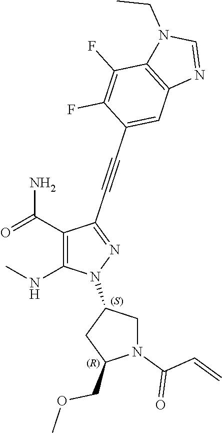

### Step 1: N-Ethyl-2,3-difluoro-6-nitroaniline

To a solution of 1,2,3-trifluoro-4-nitrobenzene (5 g, 28.23 mmol) in EtOH (40 mL) was added ethylamine (3.82 g, 84.70 mmol). The reaction mixture was stirred for 1 h 0° C. The precipitated solids were collected by filtration and washed with water (4×100 mL) and dried to afford N-ethyl-2,3-difluoro-6-nitroaniline (5.3 g, 92%) as a yellow solid which was used in the next step directly without further purification. MS ESI calculated for C₂₅H₂₄ClN₇O₂ [M−H]⁻, 201.06, found 201.05; ¹H NMR (300 MHz, CDCl₃) δ 8.00-7.92 (m, J=9.8, 5.6, 2.3 Hz, 2H), 6.53-6.39 (m, 1H), 3.71-3.62 (m, 4.7 Hz, 2H), 1.36-1.24 (m, 0.9 Hz, 3H).

### Step 2: N-Ethyl-2,3-difluoro-4-iodo-6-nitroaniline

To a stirred mixture of N-ethyl-2,3-difluoro-6-nitroaniline (5.30 g, 26.21 mmol) in AcOH (53 mL) was added NIS (6.20 g, 103.24 mmol). The reaction mixture was stirred for 16 h at 70° C. under nitrogen atmosphere. The resulting mixture was concentrated under reduced pressure. The residue was basified to pH 8 with sat. NaHCO₃ (aq.) (100 mL) at room temperature. The resulting mixture was extracted with EtOAc (2×300 mL). The combined organic layers were washed with water (2×150 mL), dried over anhydrous Na₂SO₄ and filtered. The filtrate was concentrated under reduced pressure. The residue was purified by silica gel Column chromatography, eluted with CH₂Cl₂/PE (6/1). The fractions contained desired product were combined and concentrated to afford N-ethyl-2,3-difluoro-4-iodo-6-nitroaniline (7.3 g, 84%) as a light yellow solid. MS ESI calculated for C₈H₇F₂N₂O₂[M−H]⁻, 326.95, found 326.70; H NMR (300 MHz, CDCl₃) δ 8.39 (dd, J=6.3, 2.4 Hz, 1H), 7.92 (s, 1H), 3.74-3.59 (m, 2H), 1.39-1.22 (m, 3H).

### Step 3: N1-Ethyl-5,6-difluoro-4-iodobenzene-1,2-diamine

To a stirred mixture of N-ethyl-2,3-difluoro-4-iodo-6-nitroaniline (2.00 g, 6.09 mmol) and NH₄Cl (1.63 g, 30.47 mmol) in EtOH (20.00 mL) and water (4.00 mL) was added Fe (1.36 g, 24.35 mmol). The reaction mixture was stirred for 16 h at 75° C. The resulting mixture was filtered, the filter cake was washed with EtOAc (3×100 mL). The filtrate was concentrated under reduced pressure. The residue was dissolved in water (200 mL), extracted with EA (3×100 mL). The combined organic layers were washed brine (3×100 mL), dried over anhydrous Na₂SO₄, filtered and concentrated under reduced pressure to afford N¹-ethyl-5,6-difluoro-4-iodobenzene-1,2-diamine (1.7 g, 93%) as a dark grey semi-solid which was used in the next step directly without further purification. MS ESI calculated for C₈H₉F₂IN₂[M−H]⁻, 296.98, found 296.95.

### Step 4: 1-Ethyl-6,7-difluoro-5-iodo-1,3-benzodiazole

To a stirred solution of N¹-ethyl-5,6-difluoro-4-iodobenzene-1,2-diamine (1.70 g, 5.70 mmol) in MeOH (17.00 mL) was added trimethyl orthoformate (0.91 g, 8.55 mmol) at room temperature. The reaction mixture was stirred for 16 h at 70° C. The resulting mixture was concentrated under reduced pressure. The residue was purified by silica gel Column chromatography, eluted with PE/EtOAc (1/1). The fractions contained desired product were combined and concentrated to afford 1-ethyl-6,7-difluoro-5-iodo-1,3-benzodiazole (1.3 g, 73%) as a brown yellow solid. MS ESI calculated for C₉H₇F₂IN₂[M+H]⁺, 308.96, found 309.00; H NMR (400 MHz, CDCl₃) δ 7.95 (dd, J=4.5, 1.7 Hz, 1H), 7.88 (s, 1H), 4.38 (q, J=7.3 Hz, 2H), 1.58 (t, J=7.3 Hz, 3H).

### Step 5: 3-[2-(1-Ethyl-6,7-difluoro-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a stirred mixture of 1-ethyl-6,7-difluoro-5-iodo-1,3-benzodiazole (0.13 g, 0.45 mmol), 3-ethynyl-1-[(3S,5R)-5-(methoxymethyl)-1-(prop-2-enoyl)pyrrolidin-3-yl]-5-(methylamino)pyrazole-4-carboxamide (0.15 g, 0.45 mmol), Pd(PPh₃)₂Cl₂ (31.77 mg, 0.04 mmol) and CuI (17.24 mg, 0.09 mmol) in DMF (3 mL) was added TEA (0.13 g, 1.35 mmol) dropwise at room temperature. The reaction mixture was degassed with argon for three times and stirred for 1 h at 70° C. The resulting mixture was concentrated under reduced pressure. The residue was purified by silica gel Column chromatography, eluted with CH₂Cl₂/MeOH (10/1). The fractions contained desired product were combined and concentrated. The crude product was purified by reverse flash chromatography with the following conditions: Column: XBridge Prep C18 OBD Column, 19×150 mm 5 μm; Mobile Phase A: Water (10 mmol/L NH₄HCO₃), Mobile Phase B: ACN; Flow rate: 20 mL/min; Gradient: 20 B to 50 B in 6 min, 210/254 nm. The fractions contained desired product were combined and concentrated to afford 3-[2-(1-ethyl-6,7-difluoro-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (0.12 g, 53%) as a white solid. MS ESI calculated for C₂₅H₂₇F₂N₇O₃[M+H]⁺, 512.21, found 512.20; ¹H NMR (400 MHz, CDCl₃) δ 7.92 (d, J=82.3 Hz, 2H), 6.83-6.81 (m, 2H), 6.54-6.36 (m, 2H), 5.70-5.69 (m, 1H), 5.52-5.49 (m, 1H), 5.42-5.40 (m, 1H), 4.56 (d, J=9.0 Hz, 1H), 4.39-4.33 (m, 2H), 4.10 (t, J=9.1 Hz, 1H), 4.06-3.96 (m, 1H), 3.90-3.88 (m, 1H), 3.53-3.40 (m, 1H), 3.37-3.35 (m, 3H), 3.03-3.01 (m, 3H), 2.76-2.64 (m, 1H), 2.30-2.28 (m, 1H), 1.5-1.55 (m, 3H).

### Example 126: 3-[2-(6-Chloro-1-ethyl-7-fluoro-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

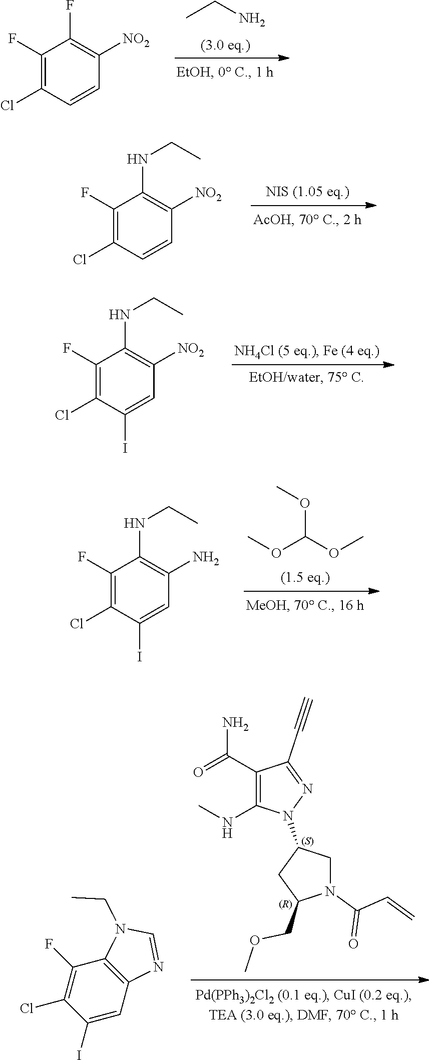

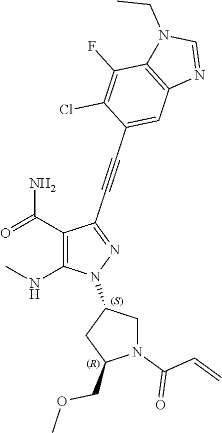

### Step 1: 3-Chloro-N-ethyl-2-fluoro-6-nitroaniline

To a stirred solution of 1-chloro-2,3-difluoro-4-nitrobenzene (3.30 g, 17.05 mmol) in EtOH (25.00 mL) was added ethylamine in EtOH (7.69 g, 51.15 mmol, 30%) dropwise at 0° C. under nitrogen atmosphere. The reaction mixture was stirred for 1 h at 0° C. under nitrogen atmosphere. The resulting mixture was filtered, the filter cake was washed with water (3×50 mL). The filter cake was dried to afford 3-chloro-N-ethyl-2-fluoro-6-nitroaniline (3.4 g, 91%) as a yellow solid which was used in the next step directly without further purification. MS ESI calculated for C₈H₈ClFN₂O₂[M+H]⁺, 219.03, found 219.05; ¹H NMR (400 MHz, CDCl₃) δ 7.93 (dd, J=9.4, 2.0 Hz, 1H), 6.64 (dd, J=9.4, 6.5 Hz, 1H), 3.72-3.61 (m, 2H), 1.33 (t, J=7.2 Hz, 3H).

### Step 2: 3-Chloro-N-ethyl-2-fluoro-4-iodo-6-nitroaniline

To a stirred solution of 3-chloro-N-ethyl-2-fluoro-6-nitroaniline (3.40 g, 15.55 mmol) in AcOH (34.00 mL) was added NIS (4.02 g, 17.88 mmol) in portions at room temperature under nitrogen atmosphere. The reaction mixture was stirred for 2 h at 70° C. under nitrogen atmosphere. The resulting mixture was concentrated under reduced pressure. The residue was diluted with EtOAc (100 mL). The resulting mixture was basified to pH 8 with saturated NaHCO₃ (aq.) and extracted with EtOAc (2×150 mL). The combined organic layers were washed with brine (100 mL), dried over anhydrous Na₂SO₄ and filtered. The filtrate was concentrated under reduced pressure. The residue was purified by silica gel column chromatography, eluted with PE/DCM (4/1). The fractions contained desired product were combined and concentrated to afford 3-chloro-N-ethyl-2-fluoro-4-iodo-6-nitroaniline (5 g, 93%) as a yellow solid. MS ESI calculated for C₈H₇ClFIN₂O₂ [M+H]⁺, 345.51, found 345.00; ¹H NMR (300 MHz, CDCl₃) δ 8.45 (d, J=2.1 Hz, 1H), 7.84 (s, 1H), 3.65 (s, 2H), 1.33 (t, J=7.2 Hz, 3H).

### Step 3: 5-Chloro-N1-ethyl-6-fluoro-4-iodobenzene-1,2-diamine

To a stirred mixture of 5-chloro-N-ethyl-3-fluoro-4-iodo-2-nitroaniline (5.00 g, 14.51 mmol) and NH₄Cl (3.88 g, 72.56 mmol) in EtOH (50.00 mL) and water (10.00 mL) was added Fe (3.24 g, 58.05 mmol) in portions at room temperature. The reaction mixture was stirred for 16 h at 75° C. under nitrogen atmosphere. The resulting mixture was filtered, the filter cake was washed with EtOAc (3×150 mL). The filtrate was diluted with water (100 mL). The resulting mixture was extracted with EtOAc (3×200 mL). The combined organic layers were washed with brine (3×80 mL), dried over anhydrous Na₂SO₄ and filtered. The filtrate was concentrated under reduced pressure to afford 5-chloro-N¹-ethyl-6-fluoro-4-iodobenzene-1,2-diamine (4.5 g, 98%) as a brown solid which was used in the next step directly without further purification. MS ESI calculated for C₈H₉ClFIN₂ [M+H]⁺, 314.95, found 315.00.

### Step 4: 6-Chloro-1-ethyl-7-fluoro-5-iodo-1H-benzo[d]imidazole

To a stirred solution of 5-chloro-N¹-ethyl-6-fluoro-4-iodobenzene-1,2-diamine (4.50 g, 14.31 mmol) in MeOH (45.00 mL) was added trimethyl orthoformate (2.28 g, 21.46 mmol) dropwise at room temperature under nitrogen atmosphere. The reaction mixture was stirred for 16 h at 70° C. under nitrogen atmosphere. The resulting mixture was concentrated under reduced pressure The residue was purified by silica gel column chromatography, eluted with CH₂Cl₂/MeOH (10/1). The fractions contained desired product were combined and concentrated to afford 6-chloro-1-ethyl-7-fluoro-5-iodo-1H-benzo[d]imidazole (4 g, 86%) as a brown solid. MS ESI calculated for C₉H₇ClFIN₂ [M+H]⁺, 324.93, found 325.00; ¹H NMR (400 MHz, CDCl₃) δ 8.11 (d, J=1.2 Hz, 1H), 7.84 (s, 1H), 4.36 (q, J=7.2 Hz, 2H), 1.57-1.54 (m, 3H).

### Step 5: 3-[2-(6-Chloro-1-ethyl-7-fluoro-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a stirred solution of 6-chloro-1-ethyl-7-fluoro-5-iodo-1,3-benzodiazole (0.15 g, 0.45 mmol), 3-ethynyl-1-[(3S,5R)-5-(methoxymethyl)-1-(prop-2-enoyl)pyrrolidin-3-yl]-5-(methylamino)pyrazole-4-carboxamide (0.15 g, 0.45 mmol), Pd(PPh₃)₂Cl₂ (31.75 mg, 0.05 mmol) and CuI (17.23 mg, 0.09 mmol) in DMF (1.50 mL) was added TEA (0.19 mL, 1.86 mmol) dropwise at room temperature. The reaction mixture was degassed with argon for three times and stirred for 1 h at 70° C. The resulting mixture was diluted with water (150 mL) and extracted with EA (3×50 mL). The combined organic layers were washed with water (5×30 mL), dried over anhydrous Na₂SO₄ and filtered. The filtrate was concentrated under reduced pressure. The residue was purified by silica gel column chromatography, eluted with CH₂Cl₂/MeOH (10/1). The fractions contained desired product were combined and concentrated. Then the crude product was purified by Prep-HPLC with the following conditions: Column: XBridge Prep C18 OBD Column, 19×150 mm 5 μm; Mobile Phase A: Water (10 mmol/L NH₄HCO₃), Mobile Phase B: ACN; Flow rate: 20 mL/min; Gradient: 15 B to 50 B in 6 min; 210/254 nm; RT: 5.75 min. The fractions contained desired product were combined and concentrated to afford 3-[2-(6-chloro-1-ethyl-7-fluoro-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (0.1 g, 43%) as a white solid. MS ESI calculated for C₂₅H₂₇ClFN₇O₃[M+H]⁺, 528.18, found 528.30; ¹H-NMR (400 MHz, CDCl₃) δ 7.95 (d, J=12 Hz, 2H), 7.29-6.53 (m, 1H), 6.51-6.43 (m, 2H), 5.75-5.71 (m, 1H), 5.56-5.41 (m, 2H), 4.60-4.37 (m, 3H), 4.16-3.90 (m, 3H), 3.53-3.39 (m, 4H), 3.05 (d, J=16 Hz, 3H), 2.78-2.29 (m, 2H), 1.62-1.57 (m, 4H).

### Example 127: 3-[2-(6,7-Difluoro-1,2-dimethyl-1,3-benzodiazol-5-yl)ethynyl]-1-[(3S,5R)-5-(methoxymethyl)-1-(prop-2-enoyl) pyrrolidin-3-yl]-5-(methylamino) pyrazole-4-carboxamide

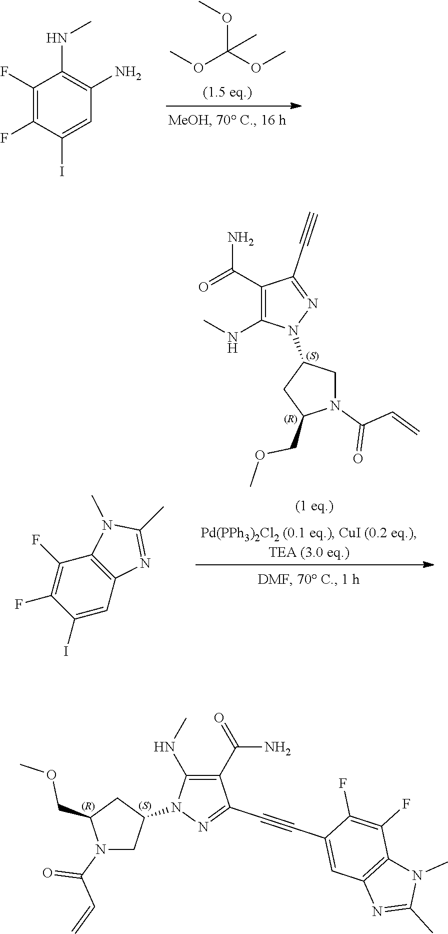

### Step 1: 6,7-Difluoro-5-iodo-1,2-dimethyl-1H-benzo[d]imidazole

To a stirred solution of 5,6-difluoro-4-iodo-N¹-methylbenzene-1,2-diamine (1.64 g, 5.77 mmol) in MeOH (18.00 mL) was added 1,1,1-trimethoxyethane (1.04 g, 8.66 mmol) at room temperature. The reaction mixture was stirred for 16 h at 70° C. The resulting mixture was concentrated under reduced pressure. The residue was purified by silica gel Column chromatography, eluted with CH₂Cl₂/MeOH (10/1). The fractions contained desired product were combined and concentrated to afford 6,7-difluoro-5-iodo-1,2-dimethyl-1H-benzo[d]imidazole (1.88 g, 82%) as a brown solid. ESI calculated for C₉H₇F₂IN₂ [M+H]⁺, 308.96, found 309.10.

### Step 2: 3-[2-(6,7-Difluoro-1,2-dimethyl-1,3-benzodiazol-5-yl)ethynyl]-1-[(3S,5R)-5-(methoxymethyl)-1-(prop-2-enoyl) pyrrolidin-3-yl]-5-(methylamino) pyrazole-4-carboxamide

To a stirred solution of 6,7-difluoro-5-iodo-1,2-dimethyl-1,3-benzodiazole (0.15 g, 0.49 mmol), 3-ethynyl-1-[(3S,5R)-5-(methoxymethyl)-1-(prop-2-enoyl) pyrrolidin-3-yl]-5-(methylamino) pyrazole-4-carboxamide (0.16 g, 0.49 mmol), Pd(PPh₃)₂Cl₂ (34.18 mg, 0.05 mmol), and CuI (18.55 mg, 0.10 mmol) in DMF (1.50 mL) was added TEA (0.20 mL, 2.01 mmol) dropwise at room temperature. The reaction mixture was degassed with argon for three times and stirred for 1 h at 70° C. The resulting mixture was diluted with EA (100 mL), washed with water (3×30 mL) and brine (50 mL), dried over anhydrous Na₂SO₄ and filtered. The filtrate was concentrated under reduced pressure. The residue was purified by silica gel Column chromatography, eluted with CH₂Cl₂/MeOH (10/1). The fractions contained desired product were combined and concentrated. The crude product was purified by Prep-HPLC with the following conditions: Column: XBridge Prep C18 OBD Column, 19×150 mm 5 μm; Mobile Phase A: Water (10 mmol/L NH₄HCO₃), Mobile Phase B: ACN; Flow rate: 20 mL/min; Gradient: 15 B to 50 B in 6 min; 210/254 nm; RT: 5.75 min. The fractions contained desired product were combined and concentrated to afford 3-[2-(6,7-difluoro-1,2-dimethyl-1,3-benzodiazol-5-yl)ethynyl]-1-[(3S,5R)-5-(methoxymethyl)-1-(prop-2-enoyl) pyrrolidin-3-yl]-5-(methylamino) pyrazole-4-carboxamide (58.5 mg, 23%) as a white solid. MS ESI calculated for C₂₅H₂₇F₂N₇O₃[M+H]⁺, 511.53, found 511.21; ¹H NMR (400 MHz, CDCl₃) δ 7.66 (d, J=4 Hz, 1H), 7.02 (s, 1H), 6.85 (s, 1H), 6.53-6.43 (m, 2H), 5.74-5.71 (m, 1H), 5.55-5.50 (m, 1H), 5.38-5.3 (m, 1H), 4.57-4.45 (m, 1H), 4.14-3.97 (m, 2H), 3.93 (s, 3H), 3.92-3.90 (m, 1H), 3.53-3.44 (m, 1H), 3.39-3.35 (m, 3H), 3.07-3.03 (m, 3H), 2.76-2.68 (m, 1H), 2.64 (s, 3H), 2.34-2.31 (m, 1H).

### Example 128: 3-[2-(6-Chloro-7-fluoro-1,2-dimethyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

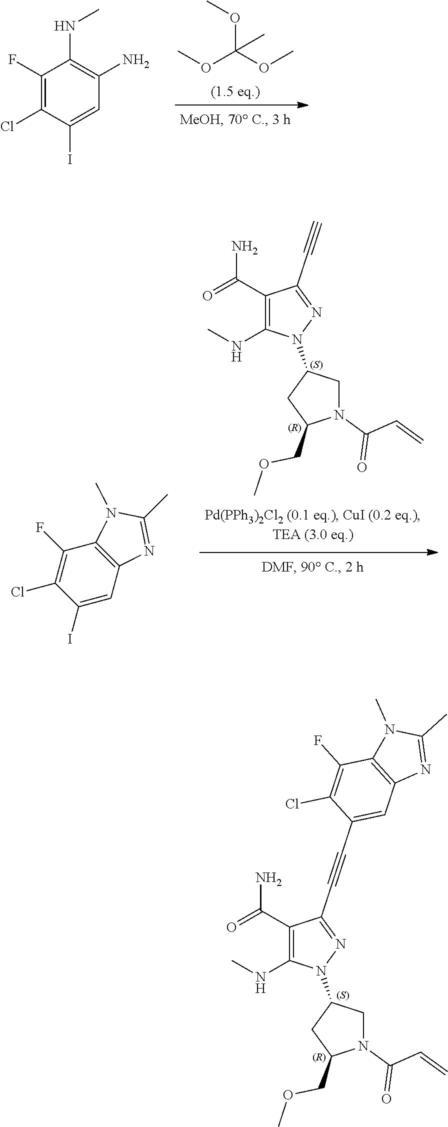

### Step 1: 6-Chloro-7-fluoro-5-iodo-1,2-dimethyl-1,3-benzodiazole

To a solution of 5-chloro-6-fluoro-4-iodo-N¹-methylbenzene-1,2-diamine (1.50 g, 4.99 mmol) in MeOH (60.00 mL) was added 1,1,1-trimethoxyethane (1.80 g, 14.98 mmol) at room temperature. The reaction mixture was stirred for 3 h at 70° C. The resulting mixture was concentrated under reduced pressure. The residue was diluted with EA (50 mL), washed with water (2×25 mL), dried over anhydrous Na₂SO₄ and filtered. The filtrate was concentrated under reduced pressure. The residue was purified by silica gel Column chromatography, eluted with EA in PE (40-100%). The fractions contained desired product were combined and concentrated to afford 6-chloro-7-fluoro-5-iodo-1,2-dimethyl-1,3-benzodiazole (0.24 g, 15%) as a yellow solid. MS ESI calculated for C₉H₇ClFIN₂ [M+H]⁺, 324.93, found 324.95; ¹H NMR (400 MHz, CDCl₃) δ 7.96 (d, J=1.2 Hz, 1H), 3.92-3.85 (m, 3H), 2.59 (s, 3H).

### Step 2: 3-[2-(6-Chloro-7-fluoro-1,2-dimethyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a stirred solution of 6-chloro-7-fluoro-5-iodo-1,2-dimethyl-1,3-benzodiazole (0.15 g, 0.45 mmol), 3-ethynyl-1-[(3S,5R)-5-(methoxymethyl)-1-(prop-2-enoyl)pyrrolidin-3-yl]-5-(methylamino)pyrazole-4-carboxamide (0.15 g, 0.45 mmol), Pd(PPh₃)₂Cl₂ (31.77 mg, 0.05 mmol) and CuI (17.24 mg, 0.09 mmol) in DMF (4.00 mL) was added TEA (0.14 g, 1.36 mmol) dropwise at room temperature. The reaction mixture was degassed with argon for three times and stirred for 2 h at 90° C. The resulting mixture was cooled down and concentrated under reduced pressure. The residue was purified by silica gel Column chromatography, eluted with MeOH in DCM (0-5%) to afford the crude product. Then the crude product was purified by Prep-HPLC with the following conditions: Column: SunFire Prep C18 OBD Column, 19×150 mm 5 μm 10 nm; Mobile Phase A: Water (0.1% FA), Mobile Phase B: ACN; Flow rate: 20 mL/min; Gradient: 25 B to 50 B in 5.8 min. The fractions contained desired product were combined and concentrated to afford 3-[2-(6-chloro-7-fluoro-1,2-dimethyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (50.1 mg, 21%) as a white solid. MS ESI calculated for C₂₅H₂₇ClFN₇O₃[M+H]⁺, 528.18, found 528.20; ¹H NMR (400 MHz, CDCl₃) δ 7.78 (s, 1H), 7.19-6.61 (m, 2H), 6.63-6.29 (m, 2H), 5.80-5.54 (m, 1H), 4.57-4.51 (m, 1H), 4.24-3.83 (m, 6H), 3.57-3.32 (m, 4H), 3.11-2.96 (m, 3H), 2.81-2.58 (m, 4H), 2.41-2.21 (m, 1H).

### Example 129: 3-[2-(6-Chloro-1-ethyl-1,3-benzodiazol-5-yl)ethynyl]-1-[(3S,5R)-1-(2-fluoroprop-2-enoyl)-5-(methoxymethyl)pyrrolidin-3-yl]-5-(methylamino)pyrazole-4-carboxamide

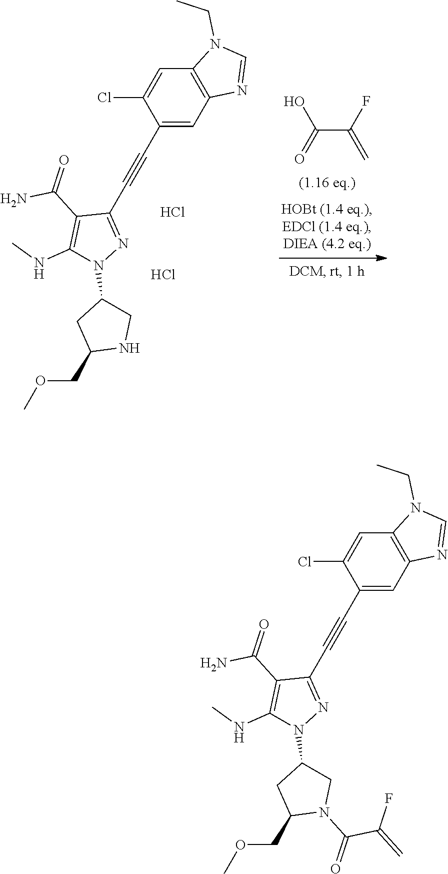

To a stirred mixture of 3-[2-(6-chloro-1-ethyl-1,3-benzodiazol-5-yl)ethynyl]-1-[(3S,5R)-5-(methoxymethyl)pyrrolidin-3-yl]-5-(methylamino)pyrazole- 4-carboxamide (90.00 mg, 0.20 mmol), 2-fluoroprop-2-enoic acid (20.62 mg, 0.23 mmol), HOBT (37.34 mg, 0.28 mmol) and EDCI (52.98 mg, 0.28 mmol) in DCM (2.00 mL) was added DIEA (0.11 g, 0.83 mmol) dropwise at 0° C. The reaction mixture was stirred for 1 h at room temperature under argon atmosphere. The resulting mixture was diluted with water (40 mL) and extracted with CH₂Cl₂ (3×30 mL). The combined organic layers were washed with brine (2×20 mL), dried over anhydrous Na₂SO₄ and filtered. The filtrate was concentrated under reduced pressure. The residue was purified by Prep-HPLC with the following conditions: Column: XBridge Prep C18 OBD Column, 19×150 mm 5 μm; Mobile Phase A: Water (10 mmol/L NH₄HCO₃), Mobile Phase B: ACN; Flow rate: 20 mL/min; Gradient: 14 B to 55 B in 6 min; 210/254 nm; RT: 5.8 min. The fractions contained desired product were combined and concentrated to afford 3-[2-(6-chloro-1-ethyl-1,3-benzodiazol-5-yl)ethynyl]-1-[(3S,5R)-1-(2-fluoroprop-2-enoyl)-5-(methoxymethyl)pyrrolidin-3-yl]-5-(methylamino)pyrazole-4-carboxamide (50.1 mg, 47%) as a white solid. MS ESI calculated for C₂₅H₂₇ClFN₇O₃[M+H]⁺, 528.18, found 528.20; ¹H NMR (400 MHz, DMSO-d₆) δ 8.52 (s, 1H), 8.02 (d, J=5.3 Hz, 2H), 7.49 (s, 1H), 6.86 (s, 1H), 5.54-5.15 (m, 3H), 4.53-4.42 (m, 1H), 4.32-4.29 (m, 2H), 4.00-3.89 (m, 2H), 3.54-3.49 (m, 2H), 3.30 (s, 3H), 2.95 (s, 3H), 2.45-2.41 (m, 1H), 2.34-2.31 (m, 1H), 1.42 (t, J=7.2 Hz, 3H).

### Example 130: 3-(2-[6-Fluoro-[1,2,4]triazolo[1,5-a]pyridin-7-yl]ethynyl)-1-[(3S,5R)-5-(methoxymethyl)-1-(prop-2-enoyl)pyrrolidin-3-yl]-5-(methylamino)pyrazole-4-carboxamide

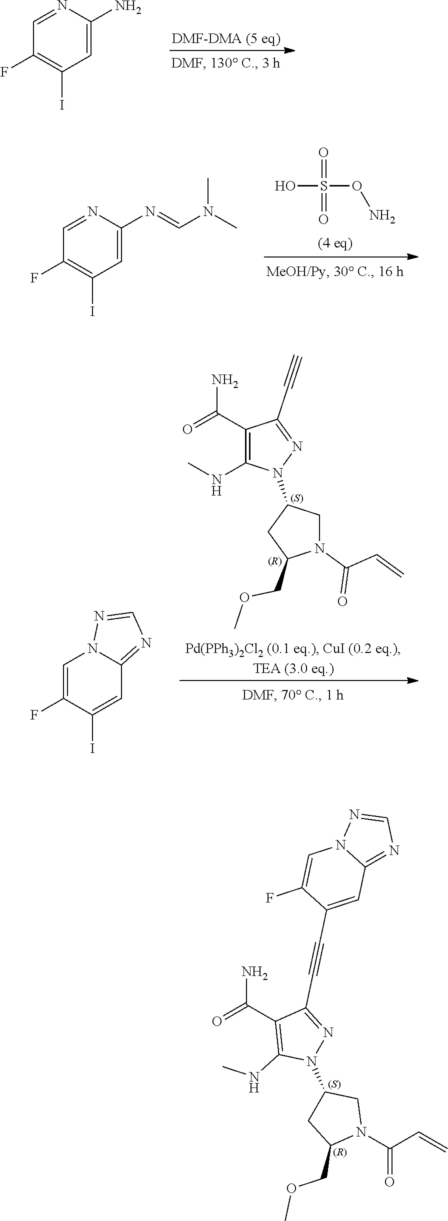

### Step 1: (E)-N-(5-Fluoro-4-iodopyridin-2-yl)-N,N-dimethylmethanimidamide

To a stirred solution of 5-fluoro-4-iodopyridin-2-amine (2.4 g, 10.25 mmol) in DMF (24.40 mL) was added DMF-DMA (6.8 mL, 51.08 mmol) at room temperature under nitrogen atmosphere. The reaction mixture was stirred for 3 h at 130° C. The resulting mixture was concentrated under reduced pressure. The residue was purified by silica gel Column chromatography, eluted with PE/EA (2/1). The fractions contained desired product were combined and concentrated to afford (E)-N-(5-fluoro-4-iodopyridin-2-yl)-N,N-dimethylmethanimidamide (2.86 g, 95%) as a light yellow oil. MS ESI calculated for C₈H₉FIN₃ [M+H]+, 293.99, found 294.35; ¹H NMR (300 MHz, CDCl₃) δ 8.36 (s, 1H), 7.98 (s, 1H), 7.45 (d, J=4.5 Hz, 1H), 3.12 (d, J=3.8 Hz, 6H).

### Step 2: 6-Fluoro-7-iodo-[1,2,4]triazolo[1,5-a]pyridine

To a stirred solution of (E)-N-(5-fluoro-4-iodopyridin-2-yl)-N,N-dimethylmethanimidamide (2.86 g, 9.76 mmol) and aminooxysulfonic acid (4.41 g, 39.03 mmol) in MeOH (28.60 mL) was added Pyridine (3.93 mL, 49.65 mmol) dropwise at 0° C. under nitrogen atmosphere. The reaction mixture was stirred for 16 h at room temperature. The resulting mixture was concentrated under reduced pressure. The residue was purified by Prep-TLC (CH₂Cl₂/MeOH 10/1). The fractions contained desired product were combined and concentrated to afford 6-fluoro-7-iodo-[1,2,4]triazolo[1,5-a]pyridine (0.30 g, 11%) as a light yellow solid. MS ESI calculated for C₆H₃FIN₃ [M+H]⁺, 263.94, found 264.00; ¹H NMR (300 MHz, CDCl₃) δ 8.56 (d, J=2.4 Hz, 1H), 8.38 (s, 1H), 8.30 (d, J=5.7 Hz, 1H).

### Step 3: 3-(2-[6-Fluoro-[1,2,4]triazolo[1,5-a]pyridin-7-yl]ethynyl)-1-[(3S,5R)-5-(methoxymethyl)-1-(prop-2-enoyl)pyrrolidin-3-yl]-5-(methylamino)pyrazole-4-carboxamide

To a stirred solution of 1-((3S,5R)-1-acryloyl-5-(methoxymethyl)pyrrolidin-3-yl)-3-ethynyl-5-(methylamino)-1H-pyrazole-4-carboxamide (0.15 g, 0.45 mmol), 6-fluoro-7-iodo-[1,2,4]triazolo[1,5-a]pyridine (0.12 g, 0.45 mmol), Pd(PPh₃)₂Cl₂ (31.59 mg, 0.05 mmol) and CuI (17.14 mg, 0.09 mmol) in DMF (2.00 mL) was added TEA (0.14 g, 1.35 mmol) at room temperature. The reaction mixture was degassed with argon for three times and stirred for 1 h at 70° C. The resulting mixture was cooled down and concentrated under reduced pressure. The residue was purified by silica gel Column chromatography, eluted with CH₂Cl₂/MeOH (10/1). The fractions contained desired product were combined and concentrated to afford crude. The crude product was purified by reverse phase flash with the following conditions Column: C18 spherical Column, 20-35 μm; Mobile Phase A: Water (10 mmol/L NH₄HCO₃), Mobile Phase B: ACN; Flow rate: 40 mL/min; Gradient: 5 B to 50 B in 20 min. The fractions contained desired product were combined and concentrated to afford 3-(2-[6-fluoro-[1,2,4]triazolo[1,5-a]pyridin-7-yl]ethynyl)-1-[(3S,5R)-5-(methoxymethyl)-1-(prop-2-enoyl)pyrrolidin-3-yl]-5-(methylamino)pyrazole-4-carboxamide (39.6 mg, 44%) as an off-white solid. MS ESI calculated for C₂₂H₂₃FN₈O₃ [M+H]⁺, 467.19, found 467.15; ¹H NMR (400 MHz, CDCl₃) δ 8.66 (s, 1H), 8.45 (s, 1H), 8.02 (s, 1H), 6.79-6.70 (m, 2H), 6.55-6.38 (m, 2H), 5.80-5.69 (m, 1H), 5.60-5.44 (m, 2H), 4.63-4.55 (m, 1H), 4.18-3.87 (m, 3H), 3.55-3.34 (m, 4H), 3.12-3.03 (m, 3H), 2.76-2.63 (m, 1H), 2.39-2.29 (m, 1H).

### Example 131: 3-[2-(6-Fluoro-1,3-benzothiazol-5-yl)ethynyl]-1-[(3S,5R)-5-(methoxymethyl)-1-(prop-2-enoyl)pyrrolidin-3-yl]-5-(methylamino)pyrazole-4-carboxamide

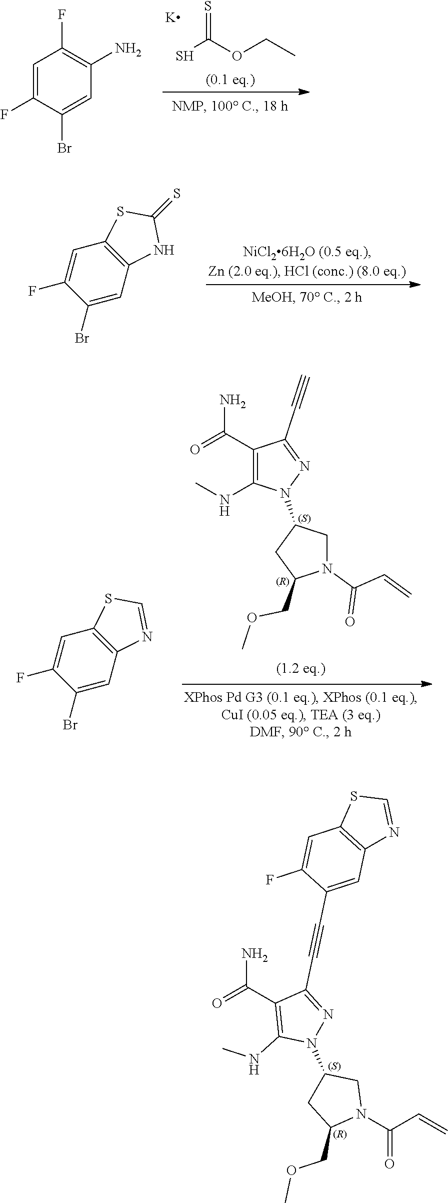

### Step 1: 5-Bromo-6-fluoro-3H-1,3-benzothiazole-2-thione

To a solution of 5-bromo-2,4-difluoroaniline (0.50 g, 2.404 mmol) in NMP (5.00 mL) was added ethoxy(potassiosulfanyl)methanethione (0.46 g, 2.885 mmol) at room temperature. The reaction mixture was stirred for 18 h at 100° C. The resulting mixture was diluted with EtOAc (10.00 mL). The residue was washed with water (3×10 mL). The combined organic layers were washed with brine (2×10 mL), dried over anhydrous Na₂SO₄ and filtered. The filtrate was concentrated under reduced pressure to afford 5-bromo-6-fluoro-3H-1,3-benzothiazole-2-thione (0.5 g, 78%) as a pink solid. MS ESI calculated for C₇H₃BrFNS₂ [M−H]⁻, 261.89, 263.89; found 262.00, 264.00; ¹H-NMR (300 MHz, CDCl₃) δ 11.41 (s, 1H), 7.25 (d, J=7.5 Hz, 2H).

### Step 2: 5-Bromo-6-fluoro-1,3-benzothiazole

To a solution of 5-bromo-6-fluoro-3H-1,3-benzothiazole-2-thione (2.60 g, 9.84 mmol), Zn (3.22 g, 49.22 mmol) and NiCl₂·6H₂O (2.46 g, 10.34 mmol) in MeOH (26.00 mL) were added con. HCl (20.00 mL) dropwise at room temperature under argon atmosphere. The reaction mixture was stirred for 2 h at 70° C. under argon atmosphere. The resulting mixture was filtered, the filter cake was washed with EtOAc (200.00 mL). The filtrate was concentrated under reduced pressure. The residue was diluted with water (200.00 mL) and basified to pH 9 with saturated NaHCO₃ (aq.). The resulting mixture was extracted with EtOAc (3×200 mL). The combined organic layers were washed with brine (300 mL), dried over anhydrous Na₂SO₄ and filtered. The filtrate was concentrated under reduced pressure. The residue was purified by silica gel column chromatography, eluted with EA in PE (0-50%). The fractions contained desired product were combined and concentrated to afford 5-bromo-6-fluoro-1,3-benzothiazole (0.41 g, 17%) as a yellow solid. ¹H NMR (300 MHz, CDCl₃) δ 8.90 (s, 1H), 8.27 (d, J=6.1 Hz, 1H), 7.64 (d, J=7.7 Hz, 1H).

### Step 3: 3-[2-(6-Fluoro-1,3-benzothiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a solution of 5-bromo-6-fluoro-1,3-benzothiazole (0.15 g, 0.64 mmol) XPhos Pd G3 (54.71 mg, 0.07 mmol), X-Phos (30.81 mg, 0.06 mmol), CuI (6.15 mg, 0.03 mmol) and 3-ethynyl-1-[(3S,5R)-5-(methoxymethyl)-1-(prop-2-enoyl)pyrrolidin-3-yl]-5-(methylamino)pyrazole-4-carboxamide (0.26 g, 0.77 mmol) in DMF (4.50 mL) was added TEA (0.20 g, 1.94 mmol) dropwise at room temperature. The reaction mixture was degassed with argon for three times and stirred for 2 h at 90° C. The resulting mixture was concentrated under reduced pressure. The residue was purified by silica gel column chromatography, eluted with MeOH in DCM (0-10%) to afford the crude product. The crude product was purified by reverse phase flash with the following conditions: Column: Spherical C18, 20-40 μm, 40 g; Mobile Phase A: Water (10 mmol/mL NH₄HCO₃); Mobile Phase B: ACN; Flow rate: 60 mL/min; Gradient: 5%-35% within 30 min; Detector: UV 254/220 nm; RT: 48 min. The fractions contained desired product were combined and concentrated to afford 3-[2-(6-fluoro-1,3-benzothiazol-5-yl)ethynyl]-1-[(3S,5R)-5-(methoxymethyl)-1-(prop-2-enoyl)pyrrolidin-3-yl]-5-(methylamino)pyrazole-4-carboxamide (95.2 mg, 30%) as a white solid. MS ESI calculated for C₂₃H₂₃FN₆O₃S [M+H]⁺, 483.15; found 483.10; ¹H-NMR (300 MHz, CDCl₃) δ 9.05 (s, 1H), 8.34 (d, J=6.1 Hz, 1H), 7.74 (d, J=8.6 Hz, 1H), 7.04 (s, 1H), 6.59-6.36 (m, 2H), 5.75-5.71 (m, 1H), 5.63-5.24 (m, 2H), 4.69-4.37 (m, 1H), 4.19-3.91 (m, 3H), 3.53-3.44 (m, 1H), 3.39 (d, J=3.6 Hz, 3H), 3.06 (d, J=11.2 Hz, 3H), 2.77-2.62 (m, 1H), 2.36-2.30 (m, 1H).

### Example 132: 1-((3S,5R)-1-Acryloyl-5-(methoxymethyl)pyrrolidin-3-yl)-3-((6-fluoroimidazo[1,2-a]pyridin-7-yl)ethynyl)-5-(methylamino)-1H-pyrazole-4-carboxamide

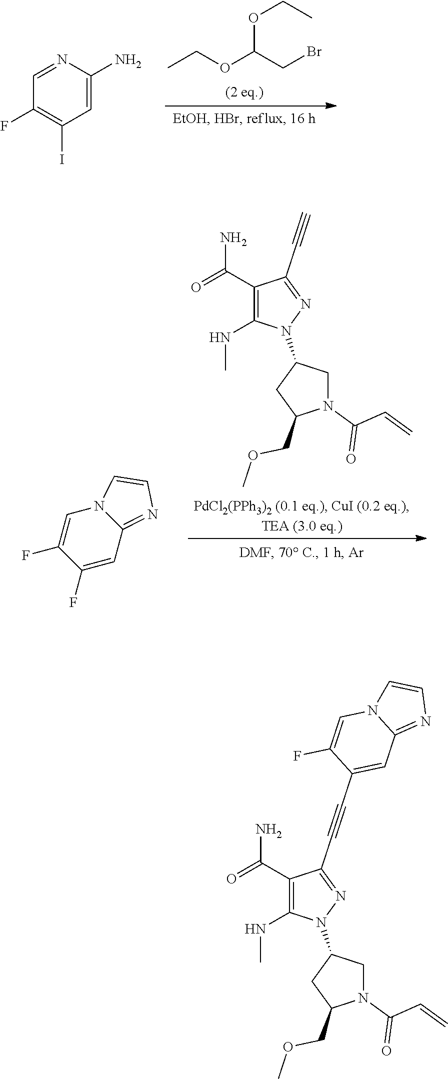

### Step 1: 6-Fluoro-7-iodoimidazo[1,2-a]pyridine

To a stirred mixture of 5-fluoro-4-iodopyridin-2-amine (0.10 g, 0.42 mmol) and 2-bromo-1,1-diethoxyethane (0.17 g, 0.84 mmol) in EtOH (0.50 mL) was added HBr (0.05 mL) dropwise at 0° C. The resulting mixture was stirred for 16 h at 80° C. The resulting mixture was cooled down to room temperature. The resulting mixture was quenched with NaHCO₃ (aq.) and extracted with EtOAc (3×60 mL). The combined organic layers were washed with brine (40 mL), dried over anhydrous Na₂SO₄ and filtered. The filtrate was concentrated under reduced pressure. The residue was purified by silica gel Column chromatography, eluted with PE/(EtOAc:EtOH (3:1)) (1/3). The fractions contained desired product were combined and concentrated to afford 6-fluoro-7-iodoimidazo[1,2-a]pyridine (1.78 g, 81%) as a brown solid. ESI calculated for C₇H₄FIN₂ [M+H]⁺, 262.94, found 262.90; ¹H NMR (400 MHz, CDCl₃) δ 8.12 (dd, J=10.0, 4.4 Hz, 2H), 7.69 (s, 1H), 7.62 (s, 1H).

### Step 2: 1-((3S,5R)-1-Acryloyl-5-(methoxymethyl)pyrrolidin-3-yl)-3-((6-fluoroimidazo[1,2-a]pyridin-7-yl)ethynyl)-5-(methylamino)-1H-pyrazole-4-carboxamide

To a stirred mixture of 6-fluoro-7-iodoimidazo[1,2-a]pyridine (0.12 g, 0.45 mmol), 3-ethynyl-1-[(3S,5R)-5-(methoxymethyl)-1-(prop-2-enoyl)pyrrolidin-3-yl]-5-(methylamino)pyrazole-4-carboxamide (0.15 g, 0.45 mmol), CuI (17.24 mg, 0.09 mmol) and Pd(PPh₃)₂Cl₂ (31.77 mg, 0.05 mmol) in DMF (1.5 mL) was added TEA (0.14 g, 1.36 mmol) dropwise at room temperature. The reaction mixture was degassed with argon for three times and stirred for 1 h at 70° C. The resulting mixture was concentrated under reduced pressure. The residue was purified by silica gel Column chromatography, eluted with CH₂Cl₂/MeOH (10/1) to afford the crude product. The crude product was purified by Prep-HPLC with the following conditions: Column: SunFire Prep C18 OBD Column, 19×150 mm 5 μm 10 nm; Mobile Phase A: Water (0.1% FA), Mobile Phase B: ACN; Flow rate: 20 mL/min; Gradient: 25 B to 50 B in 6 min; 210/254 nm; RT: 5.58 min. The fractions contained desired product were combined and concentrated to afford 1-((3S,5R)-1-acryloyl-5-(methoxymethyl)pyrrolidin-3-yl)-3-((6-fluoroimidazo[1,2-a]pyridin-7-yl)ethynyl)-5-(methylamino)-1H-pyrazole-4-carboxamide (0.15 g, 73%) as a light yellow solid. MS ESI calculated for C₂₃H₂₄FN₇O₃[M+H]⁺, 466.19, found 466.30; ¹H NMR (300 MHz, CDCl₃) δ 8.29-8.15 (m, 1H), 7.99 (d, J=6.3 Hz, 1H), 7.83 (s, 1H), 7.73 (d, J=6.6 Hz, 1H), 6.89-6.70 (m, 2H), 6.55-6.36 (m, 2H), 5.73 (dd, J=8.0, 4.3 Hz, 1H), 5.61-5.22 (m, 2H), 4.65-4.35 (m, 1H), 4.20-3.81 (m, 3H), 3.60-3.30 (m, 4H), 3.06 (d, J=10.6 Hz, 3H), 2.61-2.78 (m, 1H), 2.35-2.30 (m, 1H).

### Example 133: 3-[2-(6-Chloro-4-fluoro-1,2-dimethyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

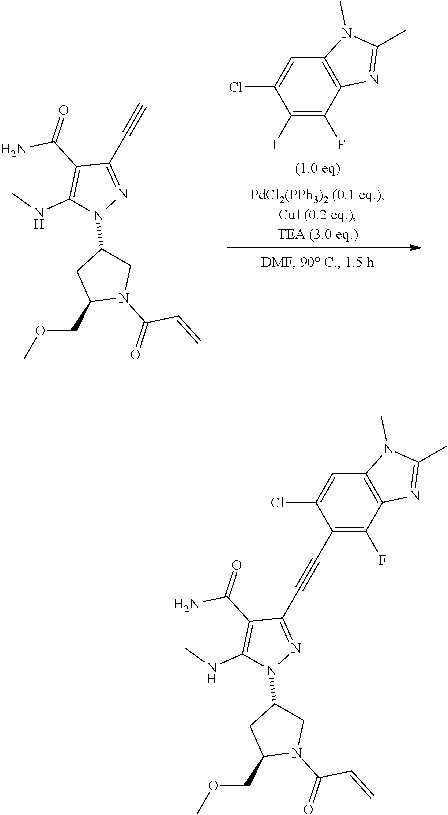

To a stirred mixture of 3-ethynyl-1-[(3S,5R)-5-(methoxymethyl)-1-(prop-2-enoyl)pyrrolidin-3-yl]-5-(methylamino)pyrazole-4-carboxamide (0.20 g, 0.60 mmol), CuI (22.99 mg, 0.12 mmol), Pd(PPh₃)₂Cl₂ (42.36 mg, 0.06 mmol) and 6-chloro-4-fluoro-5-iodo-1,2-dimethyl-1,3-benzodiazole (0.22 g, 0.66 mmol) in DMF (2 mL) was added TEA (0.25 mL, 1.81 mmol) dropwise at room temperature. The reaction mixture was degassed with argon for three times and stirred for 1.5 h at 90° C. The resulting mixture was diluted with water (150 mL) and extracted with EtOAc (3×100 mL). The combined organic layers were washed with brine (3×50 mL), dried over anhydrous Na₂SO₄ and filtered. The filtrate was concentrated under reduced pressure. The residue was purified by silica gel column chromatography, eluted with CH₂Cl₂/MeOH (10/1) to afford the crude product. The crude product was purified by Prep-HPLC with the following conditions: Column: XBridge Prep C18 OBD Column, 19×150 mm 5 μm; Mobile Phase A: Water (10 mmol/L NH₄HCO₃), Mobile Phase B: ACN; Flow rate: 20 mL/min; Gradient: 20 B to 50 B in 6 min; 210/254 nm; RT: 5.58 min. The fractions contained desired product were combined and concentrated to afford 3-[2-(6-chloro-4-fluoro-1,2-dimethyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (60.0 mg, 18%) as a white solid. MS ESI calculated for C₂₅H₂₇ClFN₇O₃[M+H]⁺, 528.18; found 528.30; ¹H NMR (300 MHz, DMSO-d₆) δ 7.80 (s, 1H), 7.58 (s, 1H), 6.95-6.49 (m, 3H), 6.18 (d, J=16.6 Hz, 1H), 5.70 (d, J=10.2 Hz, 1H), 5.50-5.00 (m, 1H), 4.47-4.35 (m, 1H), 4.13-3.98 (m, 1H), 3.97-3.82 (m, 1H), 3.77-3.74 (m, 3H), 3.62 (m, 1H), 3.47-3.42 (m, 1H), 3.38-3.27 (m, 3H), 2.98-2.95 (m, 3H), 2.57 (d, J=1.2 Hz, 3H), 2.53-2.49 (m, 1H), 2.33-2.29 (m, 1H).

### Example 134: 3-[2-(6-Chloro-1,3-benzothiazol-5-yl)ethynyl]-1-[(3S,5R)-5-(methoxymethyl)-1-(prop-2-enoyl)pyrrolidin-3-yl]-5-(methylamino)pyrazole-4-carboxamide

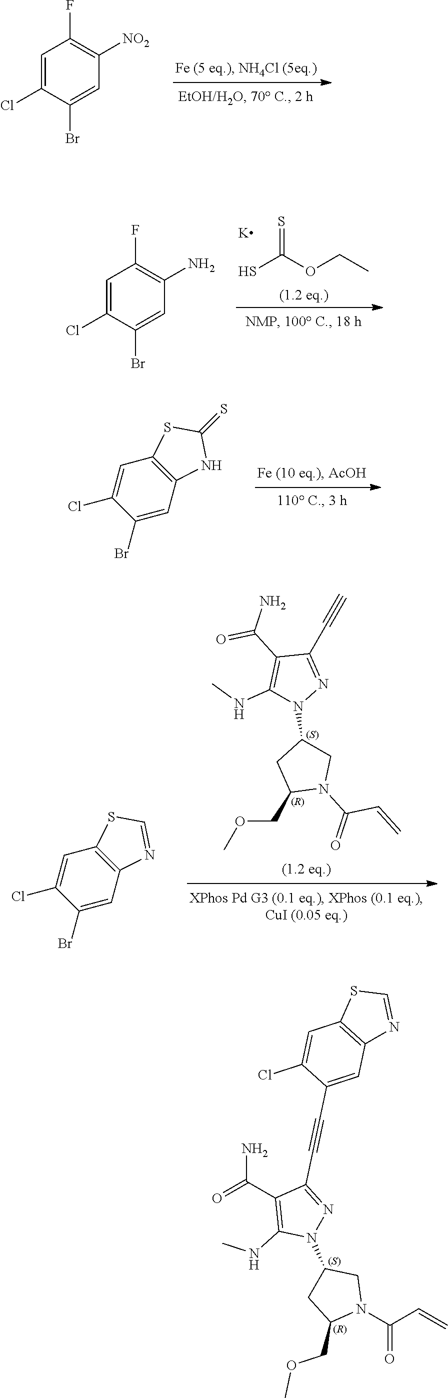

### Step 1: 5-Bromo-4-chloro-2-fluoroaniline

To a solution of 1-bromo-2-chloro-4-fluoro-5-nitrobenzene (1.00 g, 3.93 mmol) in EtOH (10.00 mL) and H₂O (2 mL) were added Fe (1.10 g, 19.65 mmol) and NH₄Cl (1.05 g, 19.65 mmol) at room temperature. The reaction mixture was stirred for 2 h at 70° C. The resulting mixture was filtered, the filter cake was washed with EA (3×150 mL). The filtrate was concentrated under reduced pressure. The residue was diluted with water (200 mL). The resulting mixture was extracted with EtOAc (3×150 mL). The combined organic layers were washed with brine (300 mL), dried over anhydrous Na₂SO₄ and filtered. The filtrate was concentrated under reduced pressure to afford 5-bromo-4-chloro-2-fluoroaniline (0.7 g, 79%) as a light yellow solid which was used in the next step directly without further purification. MS ESI calculated for C₆H₄BrClFN [M+H]⁺, 223.92, 225.92, found 223.95, 225.95; ¹H-NMR (400 MHz, CDCl₃) δ 7.12 (d, J=10.5 Hz, 1H), 7.04 (d, J=8.7 Hz, 1H).

### Step 2: 5-Bromo-6-chloro-3H-1,3-benzothiazole-2-thione

To a solution of 5-bromo-4-chloro-2-fluoroaniline (0.7 g, 3.12 mmol) in NMP (10.00 mL) was added ethoxy(potassiosulfanyl)methanethione (0.60 g, 3.74 mmol) at room temperature. The reaction mixture was stirred for 18 h at 100° C. The resulting mixture was diluted with EtOAc (150 mL) and washed with water (3×100 mL). The combined organic layers were washed with brine (200 mL), dried over anhydrous Na₂SO₄ and filtered. The filtrate was concentrated under reduced pressure. The residue was purified by silica gel column chromatography, eluted with EA in PE (0-100%). The fractions contained desired product were combined and concentrated to afford 5-bromo-6-chloro-3H-1,3-benzothiazole-2-thione (0.8 g, 91%) as an off-white solid. MS ESI calculated for C₇H₃BrClNS₂ [M−H]⁻, 277.86, 279.86, found 277.95, 279.95; ¹H NMR (400 MHz, CDCl₃) δ 17.81 (s, 1H), 8.05 (s, 1H), 7.54 (s, 1H).

### Step 3: 5-Bromo-6-chloro-1,3-benzothiazole

To a solution of 5-bromo-6-chloro-3H-1,3-benzothiazole-2-thione (0.5 g, 1.78 mmol) in AcOH (20.00 mL) was added Fe (1.00 g, 17.82 mmol) at room temperature. The reaction mixture was stirred for 3 h at 110° C. The resulting mixture was filtered; the filter cake was washed with EA (150.00 mL). The filtrate was concentrated under reduced pressure. The residue was basified to pH 9 with saturated NaHCO₃ (aq.). The resulting mixture was extracted with EtOAc (3×150 mL). The combined organic layers were washed with brine (300 mL), dried over anhydrous Na₂SO₄ and filtered. The filtrate was concentrated under reduced pressure. The residue was purified by silica gel column chromatography, eluted with EA in PE (0-80%). The fractions contained desired product were combined and concentrated to afford 5-bromo-6-chloro-1,3-benzothiazole (0.28 g, 64%) as a white solid. ¹H NMR (400 MHz, CDCl₃) δ 9.02 (s, 1H), 8.43 (s, 1H), 8.09 (s, 1H).

### Step 4: 3-[2-(6-Chloro-1,3-benzothiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a solution of 5-bromo-6-chloro-1,3-benzothiazole (0.15 g, 0.60 mmol) and 3-ethynyl-1-[(3S,5R)-5-(methoxymethyl)-1-(prop-2-enoyl)pyrrolidin-3-yl]-5-(methylamino)pyrazole-4-carboxamide (0.24 g, 0.73 mmol) in DMF (4.5 mL) were added X-Phos (28.77 mg, 0.06 mmol), XPhos Pd G3 (51.09 mg, 0.06 mmol), CuI (5.75 mg, 0.03 mmol) and TEA (0.18 g, 1.81 mmol) dropwise at room temperature. The reaction mixture was degassed with argon for three times and stirred for 2 h at 90° C. The resulting mixture was concentrated under reduced pressure. The residue was purified by silica gel column chromatography, eluted with MeOH in DCM (0-5%) to afford the crude product. The crude product was purified by Prep-HPLC with the following conditions: Column: Spherical C18, 20-40 μm, 40 g Mobile Phase A: Water (10 mmol/mL NH₄HCO₃), Mobile Phase B: ACN; Flow rate: 50 mL/min; Gradient: 5%-34% within 30 min; Detector: UV 254/220 nm; RT: 50 min. The fractions contained desired product were combined and concentrated to afford 3-[2-(6-chloro-1,3-benzothiazol-5-yl)ethynyl]-1-[(3S,5R)-5-(methoxymethyl)-1-(prop-2-enoyl)pyrrolidin-3-yl]-5-(methylamino)pyrazole-4-carboxamide (72.30 mg, 23%) as a white solid. MS ESI calculated for C₂₃H₂₃ClN₆O₃S [M+H]⁺, 499.12, 501.12, found 499.10, 501.10; ¹H-NMR (400 MHz, CDCl₃) δ 9.06 (d, J=1.6 Hz, 1H), 8.41 (d, J=5.5 Hz, 1H), 8.06 (d, J=2.3 Hz, 1H), 7.12 (s, 1H), 6.88-6.70 (m, 1H), 6.54-6.32 (m, 2H), 5.74-5.50 (m, 1H), 5.59-5.24 (m, 2H), 4.59-4.57 (m, 1H), 4.15-3.99 (m, 2H), 3.93-3.90 (m, 1H), 3.56-3.44 (m, 1H), 3.39-3.36 (m, 3H), 3.07-3.02 (m, 3H), 2.74-2.68 (m, 1H), 2.35-2.30 (m, 1H).

### Example 135: 3-[2-(1-Cyclopropyl-6-fluoro-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

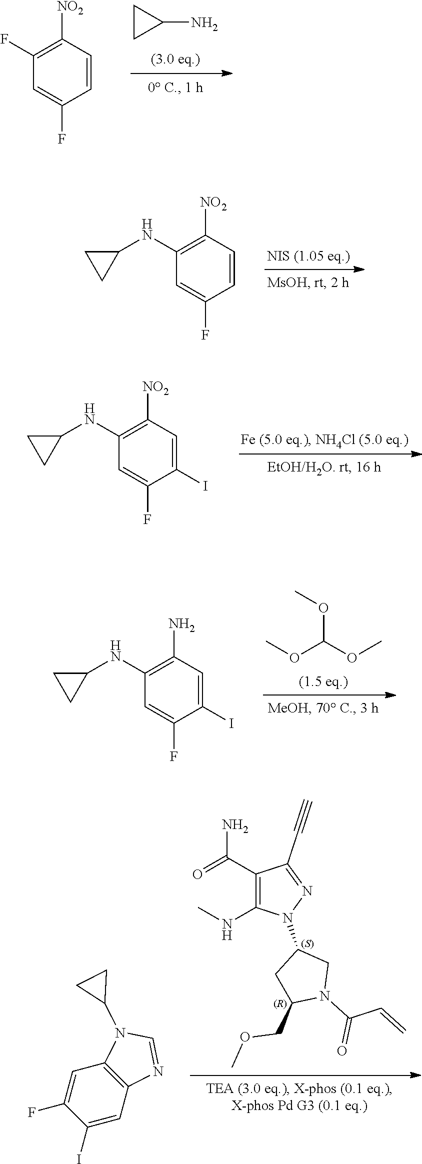

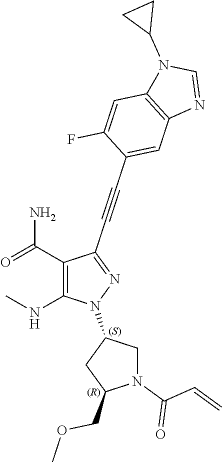

### Step 1: N-cyclopropyl-5-fluoro-2-nitroaniline

To a stirred solution of 2,4-difluoro-1-nitrobenzene (20 g, 125.71 mmol) in EtOH (200.00 mL) was added aminocyclopropane (21.53 g, 377.14 mmol) dropwise at 0° C. The reaction mixture was stirred for 1 h at 0° C. The resulting mixture was diluted with H2O (400 mL) and filtered. The filter cake was washed with H2O (3×200 mL). The filter cake was dried under reduced pressure to afford N-cyclopropyl-5-fluoro-2-nitroaniline (25.5 g, crude) as a yellow solid which was used in the next step directly without further purification. MS ESI calculated for C9H9FN2O2 [M+H]+, 197.06, found 196.95.

### Step 2: N-cyclopropyl-5-fluoro-4-iodo-2-nitroaniline

To a stirred solution of N-cyclopropyl-5-fluoro-2-nitroaniline (25.5 g, 129.98 mmol) in methanesulfonic acid (100 mL) was added NIS (30.71 g, 136.48 mmol) at 0° C. The reaction mixture was stirred for 2 h at room temperature. The resulting mixture was diluted with water (500 mL) and extracted with EtOAc (3×300 mL). The combined organic layers was washed with brine (500 mL), dried over anhydrous Na₂SO₄ and filtered. The filtrate was concentrated under reduced pressure. The residue was purified by silica gel column chromatography, eluted with EA in PE (0-10%). The fractions contained desired product were combined and concentrated to afford N-cyclopropyl-5-fluoro-4-iodo-2-nitroaniline (27 g, 64%) as a black solid. MS ESI calculated for C9H8FIN2O2 [M+H]+, 322.96, found, 322.95.

### Step 3: N1-cyclopropyl-5-fluoro-4-iodobenzene-1,2-diamine

To a stirred mixture of N-cyclopropyl-5-fluoro-4-iodo-2-nitroaniline (27.0 g, 83.83 mmol) and NH4Cl (22.42 g, 419.16 mmol) in EtOH (150 mL) and H2O (150 mL) was added Fe (23.41 g, 419.16 mmol) at room temperature. The reaction mixture was stirred for 16 h at room temperature. The resulting mixture was filtered. The filter cake was washed with MeOH (3×100 mL). The filtrate was concentrated under reduced pressure to afford N1-cyclopropyl-5-fluoro-4-iodobenzene-1,2-diamine (23 g, crude) as a light brown solid which was used in the next step directly without further purification. MS ESI calculated for C9H10FIN2 [M+H]+, 292.99, found 292.95.

### Step 4: 1-Cyclopropyl-6-fluoro-5-iodo-1,3-benzodiazole

To a stirred solution of N1-cyclopropyl-5-fluoro-4-iodobenzene-1,2-diamine (23 g, 78.74 mmol) in MeOH (230 mL) was added trimethyl orthoformate (25.07 mL, 236.22 mmol) at room temperature under argon atmosphere. The reaction mixture was stirred for 3 h at 70° C. The resulting mixture was concentrated under reduced pressure. The residue was purified by silica gel column chromatography, eluted with EA in PE (50%-100%). The fractions contained desired product were combined and concentrated to afford 1-cyclopropyl-6-fluoro-5-iodo-1,3-benzodiazole (8 g, 34%) as a yellow solid. MS ESI calculated for C10H8FIN2 [M+H]+, 302.97, found 302.85. 1H NMR (400 MHz, CDCl3) δ 8.15 (d, J=5.6 Hz, 1H), 7.92 (s, 1H), 7.32 (d, J=7.6 Hz, 1H), 3.36-3.31 (m, 1H), 1.26-1.13 (m, 2H), 1.15-1.01 (m, 2H).

### Step 5: 3-[2-(1-Cyclopropyl-6-fluoro-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

To a stirred solution of 3-ethynyl-1-[(3S,5R)-5-(methoxymethyl)-1-(prop-2-enoyl)pyrrolidin-3-yl]-5-(methylamino)pyrazole-4-carboxamide (0.77 g, 2.32 mmol) and 1-cyclopropyl-6-fluoro-5-iodo-1,3-benzodiazole (0.70 g, 2.32 mmol) in DMF (20 mL) were added XPhos (0.11 g, 0.23 mmol), X-Phos Pd G3 (0.20 g, 0.23 mmol), CuI (44.13 mg, 0.23 mmol) at room temperature under nitrogen atmosphere. To the above mixture was added TEA (0.10 g, 0.99 mmol) dropwise over 1 min at room temperature. The reaction mixture was degassed with nitrogen for three times and stirred for 1 h at 90° C. The resulting mixture was cooled down to room temperature. The residue was purified by silica gel column chromatography, eluted with CH2Cl2/MeOH (10/1) to afford crude product. The crude product was further purified by reverse flash chromatography with the following conditions: column, C18 silica gel; mobile phase, ACN in water, 10% to 50% gradient in 30 min; detector, UV 254 nm. The fractions contained desired product were combined and concentrated to afford 3-[2-(1-cyclopropyl-6-fluoro-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide (0.63 g, 52%) as an off-white solid. MS ESI calculated for C26H28FN7O3 [M+H]+, 506.23; found 506.20. 1H NMR (300 MHz, DMSO-d6) δ 8.38 (s, 1H), 7.97 (d, J=6.3 Hz, 1H), 7.66 (d, J=9.5 Hz, 1H), 7.50 (s, 1H), 6.92-6.45 (m, 3H), 6.24-6.13 (m, 1H), 5.70 (dd, J=10.2, 2.7 Hz, 1H), 5.30-5.26 (m, 1H), 4.48-4.42 (m, 1H), 4.05-4.01 (m, 1H), 3.91-3.87 (m, 1H), 3.66-3.43 (m, 3H), 3.32 (d, J=3.9 Hz, 3H), 2.98-2.91 (m, 3H), 2.68-2.62 (m, 1H), 2.33-2.28 (m, 1H), 1.13-1.03 (m, 4H).

### Example 136: 3-[2-(1-Cyclopropyl-6-fluoro-1,3-benzodiazol-5-yl)ethynyl]-5-(methylamino)-1-[(3S)-1-(prop-2-enoyl)pyrrolidin-3-yl]pyrazole-4-carboxamide

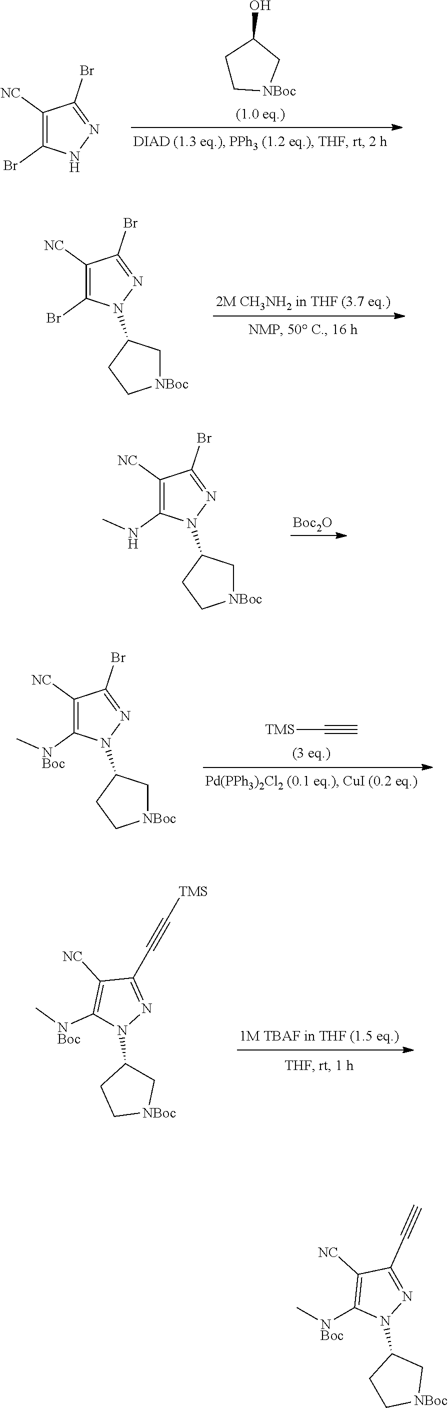

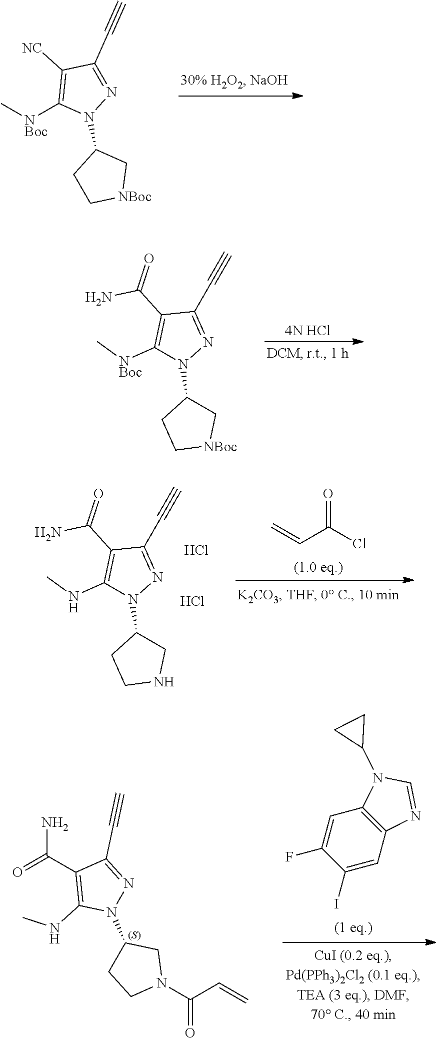

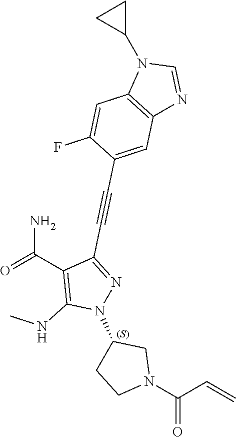

### Step 1: Tert-butyl(3S)-3-(3,5-dibromo-4-cyanopyrazol-1-yl)pyrrolidine-1-carboxylate

To a stirred solution of 3,5-dibromo-1H-pyrazole-4-carbonitrile (5 g, 19.93 mmol) and tert-butyl(R)-3-hydroxypyrrolidine-1-carboxylate (4.48 g, 23.91 mmol) in THF (100 mL) was added PPh3 (7.84 g, 29.89 mmol) and DIAD (6.04 g, 29.89 mmol) dropwise at 0° C. under argon atmosphere. The reaction mixture was degassed with argon for three times and stirred for 2 h at room temperature. The resulting mixture was concentrated under reduced pressure. The residue was purified by silica gel column chromatography, eluted with PE/EA (2/1). The fractions contained desired product were combined and concentrated to afford tert-butyl(3S)-3-(3,5-dibromo-4-cyanopyrazol-1-yl)pyrrolidine-1-carboxylate (8 g, 95%) as a white solid. MS ESI calculated for C13H16Br2N4O2 [M+H−56]+, 362.96, 364.96, 366.96, found 362.95, 364.95, 366.95; 1H NMR (400 MHz, CDCl3) δ 3.90-3.31 (m, 4H), 2.52-2.26 (m, 2H), 2.09-2.00 (m, 1H), 1.46 (s, 9H).

### Step 2: Tert-butyl(3S)-3-[3-bromo-4-cyano-5-(methylamino)pyrazol-1-yl]pyrrolidine-1-carboxylate

To a stirred solution of tert-butyl(3S)-3-(3,5-dibromo-4-cyanopyrazol-1-yl)pyrrolidine-1-carboxylate (9 g, 21.42 mmol) in NMP (90 mL) was added CH3NH2 in THF (2.46 g, 79.26 mmol) at room temperature. The reaction mixture was stirred for 16 h at 50° C. The resulting mixture was concentrated under reduced pressure. The residue was purified by reverse flash chromatography with the following conditions: column, C18 silica gel; mobile phase, MeCN in water (NH4HCO3), 30% to 60% gradient in 30 min; detector, UV 254 nm. The fractions contained desired product were combined and concentrated to afford tert-butyl(3S)-3-[3-bromo-4-cyano-5-(methylamino)pyrazol-1-yl]pyrrolidine-1-carboxylate (2.7 g, 34%) as a white solid. MS ESI calculated for C14H20BrN5O2 [M−H]−, 368.08, 370.08, found 368.10, 370.10; 1H NMR (400 MHz, CDCl3) δ 5.11 (s, 1H), 4.53-4.33 (m, 1H), 3.76-3.60 (m, 2H), 3.58 (d, J=9.9 Hz, 1H), 3.51-3.36 (m, 1H), 3.18 (s, 3H), 2.49-2.45 (m, 1H), 2.24-1.96 (m, 1H), 1.46 (s, 9H).

### Step 3: Tert-butyl(3S)-3-{3-bromo-5-[(tert-butoxycarbonyl)(methyl)amino]-4-cyanopyrazol-1-yl}pyrrolidine-1-carboxylate

To a stirred solution of tert-butyl(3S)-3-[3-bromo-4-cyano-5-(methylamino)pyrazol-1-yl]pyrrolidine-1-carboxylate (6.3 g, 17.01 mmol) and TEA (2.58 g, 25.52 mmol) in DCM (60 mL) were added DMAP (0.21 g, 1.70 mmol) and Boc₂O (7.43 g, 34.03 mmol) dropwise at room temperature. The reaction mixture was stirred for 1 h at room temperature. The resulting mixture was concentrated under reduced pressure. The residue was purified by silica gel column chromatography, eluted with PE/EA (2/1). The fractions contained desired product were combined and concentrated to afford tert-butyl(3S)-3-{3-bromo-5-[(tert-butoxycarbonyl)(methyl)amino]-4-cyanopyrazol-1-yl}pyrrolidine-1-carboxylate (7.60 g, 94%) as a white solid. MS ESI calculated for C19H28BrN5O4 [M+H]+, 470.13, 472.13, found 470.25, 472.25; 1H NMR (400 MHz, CDCl3) δ 3.72-3.62 (m, 3H), 3.52-3.26 (m, 2H), 3.21 (s, 3H), 2.47-2.16 (m, 2H), 1.44 (s, 18H).

### Step 4: Tert-butyl(3S)-3-{5-[(tert-butoxycarbonyl)(methyl)amino]-4-cyano-3-[2-(trimethylsilyl)ethynyl]pyrazol-1-yl}pyrrolidine-1-carboxylate

To a stirred solution of tert-butyl(3S)-3-{3-bromo-5-[(tert-butoxycarbonyl)(methyl)amino]-4-cyanopyrazol-1-yl}pyrrolidine-1-carboxylate (7 g, 14.88 mmol), Pd(PPh3)2Cl2 (1.04 g, 1.48 mmol) and CuI (0.57 g, 2.97 mmol) in DMF (70 mL) were added TEA (6.21 mL, 44.64 mmol) and trimethylsilylacetylene (4.39 g, 44.64 mmol) dropwise at room temperature. The reaction mixture was degassed with argon for three times and stirred for 2 h at 90° C. The resulting mixture was concentrated under reduced pressure. The residue was purified by silica gel column chromatography, eluted with PE/EA (2/1). The fractions contained desired product were combined and concentrated to afford tert-butyl (3S)-3-{5-[(tert-butoxycarbonyl)(methyl)amino]-4-cyano-3-[2-(trimethylsilyl)ethynyl]pyrazol-1-yl}pyrrolidine-1-carboxylate (7 g, 96%) as a brown solid. MS ESI calculated for C24H37N5O4Si [M+H]+, 488.26, found 488.35; 1H NMR (300 MHz, CDCl3) δ 3.68-3.33 (m, 4H), 3.22-2.96 (m, 3H), 2.64-1.90 (m, 3H), 1.44 (s, 18H), 0.27 (s, 9H).

### Step 5: Tert-butyl(3S)-3-{5-[(tert-butoxycarbonyl)(methyl)amino]-4-cyano-3-ethynylpyrazol-1-yl}pyrrolidine-1-carboxylate

To a stirred solution of tert-butyl(3S)-3-{5-[(tert-butoxycarbonyl)(methyl)amino]-4-cyano-3-[2-(trimethylsilyl)ethynyl]pyrazol-1-yl}pyrrolidine-1-carboxylate (7.00 g, 14.35 mmol) in THF (70 mL) was added TBAF (21.53 mL, 21.53 mmol, 1 M in THF) at room temperature. The reaction mixture was stirred for 1 h at room temperature. The resulting mixture was concentrated under reduced pressure. The residue was purified by silica gel column chromatography, eluted with PE/EA (3/1). The fractions contained desired product were combined and concentrated to afford tert-butyl(3S)-3-{5-[(tert-butoxycarbonyl)(methyl)amino]-4-cyano-3-ethynylpyrazol-1-yl}pyrrolidine-1-carboxylate (4.30 g, 72%) as a white solid. MS ESI calculated for C21H29N5O4 [M+H]+, 416.22, found 416.30; 1H NMR (400 MHz, CDCl3) δ 3.77-3.73 (m, 2H), 3.60-3.50 (m, 1H), 3.51-3.47 (m, 2H), 3.38 (s, 1H), 3.23 (s, 3H), 2.43-2.39 (m, 1H), 2.32-2.28 (m, 1H), 1.46 (s, 18H).

### Step 6: Tert-butyl(3S)-3-{5-[(tert-butoxycarbonyl)(methyl)amino]-4-carbamoyl-3-ethynylpyrazol-1-yl}pyrrolidine-1-carboxylate

To a stirred solution of tert-butyl(3S)-3-{5-[(tert-butoxycarbonyl)(methyl)amino]-4-cyano-3-ethynylpyrazol-1-yl}pyrrolidine-1-carboxylate (3.70 g, 8.90 mmol) and NaOH (20.48 mL, 10.24 mmol, 0.5 M) in EtOH (30 mL) and DMSO (6 mL) was added H₂O₂ (0.73 mL, 31.16 mmol, 30%) dropwise at 0° C. The reaction mixture was stirred for 30 min at 0° C. then 40 min at room temperature. The resulting mixture was concentrated under reduced pressure. The residue was purified by silica gel column chromatography, eluted with CH2Cl2/MeOH (10/1). The fractions contained desired product were combined and concentrated to afford tert-butyl(3S)-3-{5-[(tert-butoxycarbonyl)(methyl)amino]-4-carbamoyl-3-ethynylpyrazol-1-yl}pyrrolidine-1-carboxylate (3.30 g, 85%) as a white solid. MS ESI calculated for C21H31N5O5 [M+H]+, 434.24, found 434.50; 1H NMR (300 MHz, CDCl3) δ 6.81 (brs, 1H), 6.16 (brs, 1H), 4.86-4.82 (m, 1H), 3.74-3.69 (m, 2H), 3.51-3.45 (m, 1H), 3.45-3.40 (m, 1H), 3.12 (s, 3H), 2.98-2.93 (m, 2H), 2.27-2.22 (m, 1H), 1.53-1.29 (m, 18H).

### Step 7: 3-Ethynyl-5-(methylamino)-1-[(3S)-pyrrolidin-3-yl]pyrazole-4-carboxamide dihydrochloride

To a stirred mixture of tert-butyl(3S)-3-{5-[(tert-butoxycarbonyl)(methyl)amino]-4-carbamoyl-3-ethynylpyrazol-1-yl}pyrrolidine-1-carboxylate (3.00 g, 6.92 mmol) in DCM (30 mL) was added HCl (gas) in 1,4-dioxane (15 mL) dropwise at room temperature. The resulting mixture was stirred for 1 h at room temperature. The resulting mixture was filtered. The filter cake was washed with DCM (3×20 mL) and dried to afford 3-ethynyl-5-(methylamino)-1-[(3S)-pyrrolidin-3-yl]pyrazole-4-carboxamide dihydrochloride (2.30 g, crude) as an off-white solid. MS ESI calculated for C11H17Cl2N5O [M+H]+, 306.08, found 306.60.

### Step 8: 3-Ethynyl-5-(methylamino)-1-[(3S)-1-(prop-2-enoyl)pyrrolidin-3-yl]pyrazole-4-carboxamide

To a stirred solution of 3-ethynyl-5-(methylamino)-1-[(3S)-pyrrolidin-3-yl]pyrazole-4-carboxamide dihydrochloride (2.20 g, 7.18 mmol) and K2CO3 (34.49 mL, 86.22 mmol, 2.5 M) in THF (22 mL) was added acryloyl chloride (0.59 g, 6.46 mmol) dropwise at 0° C. The reaction mixture was stirred for 10 min at 0° C. The resulting mixture was diluted with water (50 mL) and extracted with EtOAc (3×50 mL). The combined organic layers was washed with brine (150 mL), dried over anhydrous Na2SO4, filtered and concentrated under reduced pressure. The residue was purified by silica gel column chromatography, eluted with CH2Cl2/MeOH (10/1). The fractions contained desired product were combined and concentrated to afford 3-ethynyl-5-(methylamino)-1-[(3S)-1-(prop-2-enoyl)pyrrolidin-3-yl]pyrazole-4-carboxamide (1.20 g, 58%) as a white solid. MS ESI calculated for C14H17N5O2 [M+H]+, 288.14, found 288.05; 1H NMR (400 MHz, DMSO-d6) δ 6.69-6.47 (m, 2H), 6.15-5.96 (m, 1H), 5.68-5.42 (m, 1H), 5.24-5.01 (m, 1H), 4.57-4.21 (m, 1H), 3.88-3.65 (m, 3H), 3.62-3.50 (m, 1H), 2.52-2.48 (m, 4H), 2.41-2.20 (m, 2H).

### Step 9: 3-[2-(1-Cyclopropyl-6-fluoro-1,3-benzodiazol-5-yl)ethynyl]-5-(methylamino)-1-[(3S)-1-(prop-2-enoyl)pyrrolidin-3-yl]pyrazole-4-carboxamide

To a stirred mixture of 3-ethynyl-5-(methylamino)-1-[(3S)-1-(prop-2-enoyl)pyrrolidin-3-yl]pyrazole-4-carboxamide (0.13 g, 0.45 mmol), 1-cyclopropyl-6-fluoro-5-iodo-1,3-benzodiazole (0.13 g, 0.45 mmol), Pd(PPh3)2Cl2 (31.76 mg, 0.04 mmol) and CuI (17.23 mg, 0.09 mmol) in DMF (1.5 mL) was added TEA (0.13 g, 1.35 mmol) dropwise at room temperature under argon atmosphere. The reaction mixture was degassed with argon for three times and stirred for 40 min at 70° C. The resulting mixture was concentrated under reduced pressure. The residue was purified by silica gel column chromatography, eluted with CH2Cl2/MeOH (10/1) to afford the crude product which was purified by Prep-HPLC with the following conditions: Column: XBridge Prep C18 OBD Column, 19×150 mm 5 μm; Mobile Phase A: water (10 mmol/L NH4HCO3), Mobile Phase B: ACN; Flow rate: 20 mL/min; Gradient: 20 B to 50 B in 6 min; 210/254 nm; RT: 5.58 min; The fractions contained desired product were combined and concentrated to afford 3-[2-(1-cyclopropyl-6-fluoro-1,3-benzodiazol-5-yl)ethynyl]-5-(methylamino)-1-[(3S)-1-(prop-2-enoyl)pyrrolidin-3-yl]pyrazole-4-carboxamide (0.10 g, 50%) as a white solid. MS ESI calculated for C24H24FN7O2 [M+H]+, 462.20, found 462.15; 1H NMR (300 MHz, DMSO-d6) δ 8.37 (s, 1H), 7.98-7.75 (m, 1H), 7.65-7.51 (m, 1H), 7.49 (s, 1H), 6.92-6.51 (m, 3H), 6.25-6.11 (m, 1H), 5.70-5.42 (m, 1H), 5.19-4.96 (m, 1H), 4.14-3.44 (m, 5H), 2.97-2.54 (m, 3H), 2.36-1.96 (m, 2H), 1.18-1.00 (m, 4H).

### Example 137: 1-[(3S,5R)-1-(but-2-ynoyl)-5-(methoxymethyl)pyrrolidin-3-yl]-3-[2-(1-ethyl-4,6-difluoro-1,3-benzodiazol-5-yl)ethynyl]-5-(methylamino)pyrazole-4-carboxamide

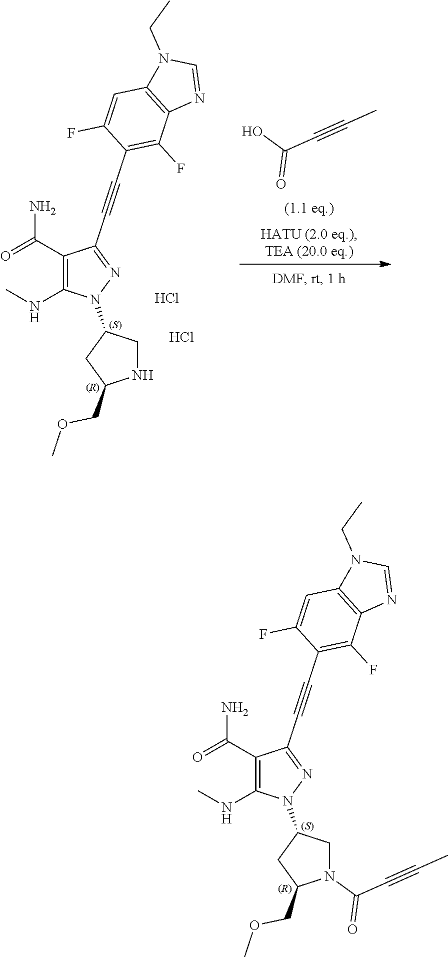

1-[(3S,5R)-1-(but-2-ynoyl)-5-(methoxymethyl)pyrrolidin-3-yl]-3-[2-(1-ethyl-4,6-difluoro-1,3-benzodiazol-5-yl)ethynyl]-5-(methylamino)pyrazole-4-carboxamide. MS ESI calculated for C₂₆H₂₇F₂N₇O₃[M+H]⁺, 524.21, found 524.35; ¹H NMR (300 MHz, CD₃OD) δ 8.33 (s, 1H), 7.45 (d, J=8.9 Hz, 1H), 5.48-5.34 (m, 1H), 4.64-4.49 (m, 1H), 4.35-4.30 (m, 2H), 4.12-3.68 (m, 3H), 3.68-3.58 (m, 1H), 3.43-3.40 (m, 1H), 3.42-3.39 (m, 3H), 3.04-3.01 (m, 3H), 2.84-2.61 (m, 1H), 2.47-2.43 (m, 1H), 2.06-2.03 (m, 3H), 1.53 (t, J=7.3 Hz, 3H).

### Example 138: 1-((3S,5R)-1-acryloyl-5-((methoxy-d3)methyl)pyrrolidin-3-yl)-3-((6-chloro-1-ethyl-1H-benzo[d]imidazol-5-yl)ethynyl)-5-(methylamino)-1H-pyrazole-4-carboxamide

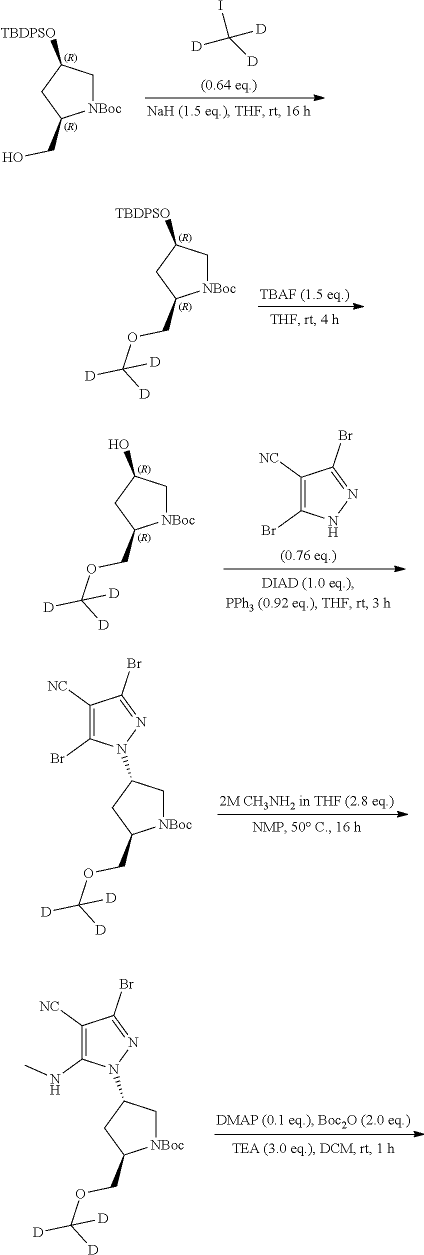

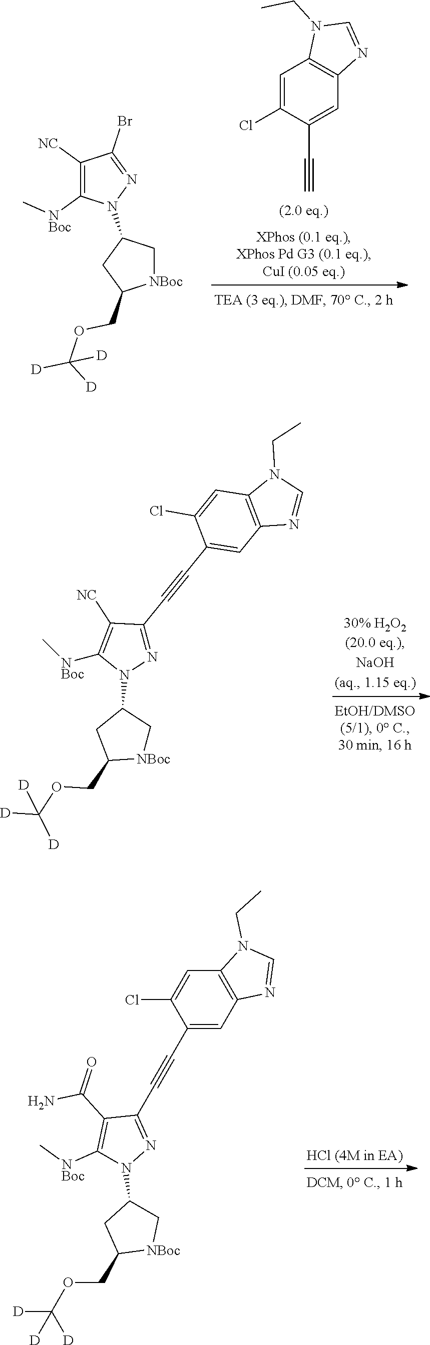 [SMILES: [2H]C([2H])([2H])OC[C@H]1C[C@H](n2nc(Br)c(C#N)c2N(C)C(=O)OC(C)(C)C)CN1C(=O)OC(C)(C)C ; C#Cc1cc2ncn(CC)c2cc1Cl ; [2H]C([2H])([2H])OC[C@H]1C[C@H](n2nc(C#Cc3cc4ncn(CC)c4cc3Cl)c(C#N)c2N(C)C(=O)OC(C)(C)C)CN1C(=O)OC(C)(C)C ; [2H]C([2H])([2H])OC[C@H]1C[C@H](n2nc(C#Cc3cc4ncn(CC)c4cc3Cl)c(C(N)=O)c2N(C)C(=O)OC(C)(C)C)CN1C(=O)OC(C)(C)C]

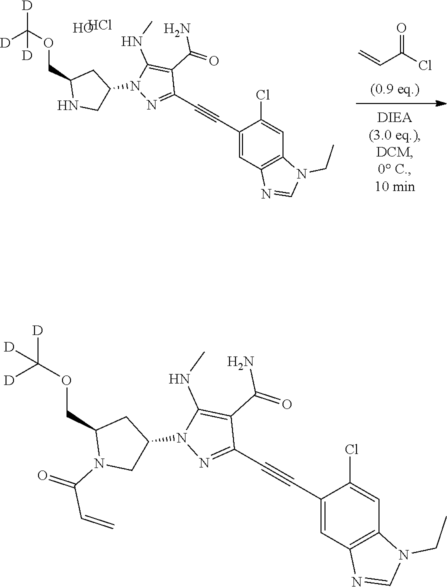

1-((3S,5R)-1-acryloyl-5-((methoxy-d₃)methyl)pyrrolidin-3-yl)-3-((6-chloro-1-ethyl-1H-benzo[d]imidazol-5-yl)ethynyl)-5-(methylamino)-1H-pyrazole-4-carboxamide. MS ESI calculated for C₂₅H₂₅D₃ClN₇O₃[M+H]+, 531.21, found 531.25; ¹H NMR (400 MHz, CDCl₃) δ 8.10 (d, J=5.1 Hz, 1H), 8.00 (s, 1H), 7.51 (s, 1H), 6.82 (s, 1H), 6.57-6.36 (m, 2H), 5.72 (dd, J=8.6, 3.6 Hz, 1H), 5.59-5.25 (m, 2H), 4.57-4.45 (m, 1H), 4.29-3.87 (m, 4H), 3.55-3.42 (m, 1H), 3.09-2.95 (m, 3H), 2.73-2.69 (m, 1H), 2.36-2.31 (m, 1H), 1.58-1.50 (m, 3H).

### Example 139: 3-[2-(6-Fluoro-1-methyl-1,3-benzodiazol-5-yl)ethynyl]-5-(methylamino)-1-[(3 S)-1-(prop-2-enoyl)pyrrolidin-3-yl]pyrazole-4-carboxamide

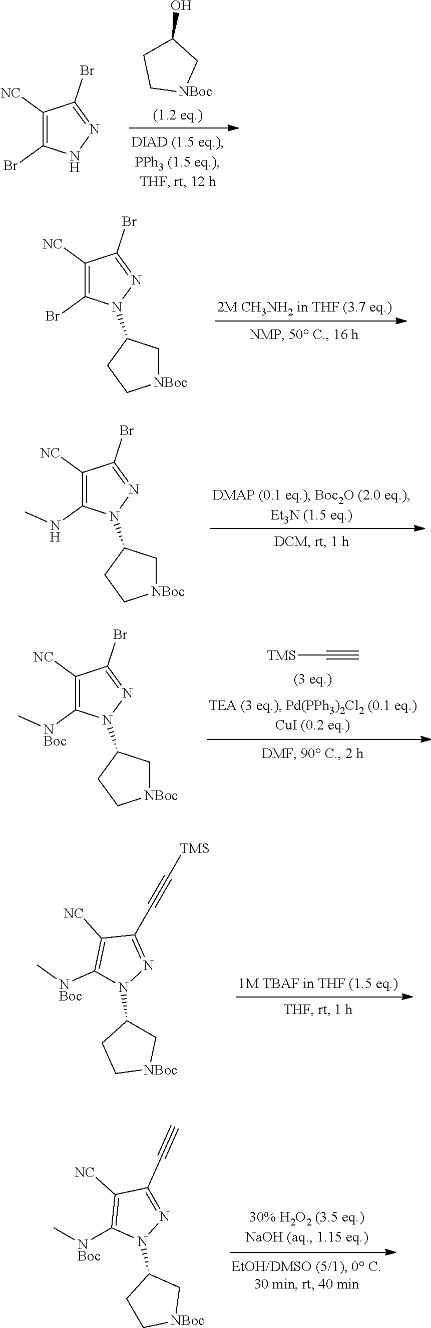

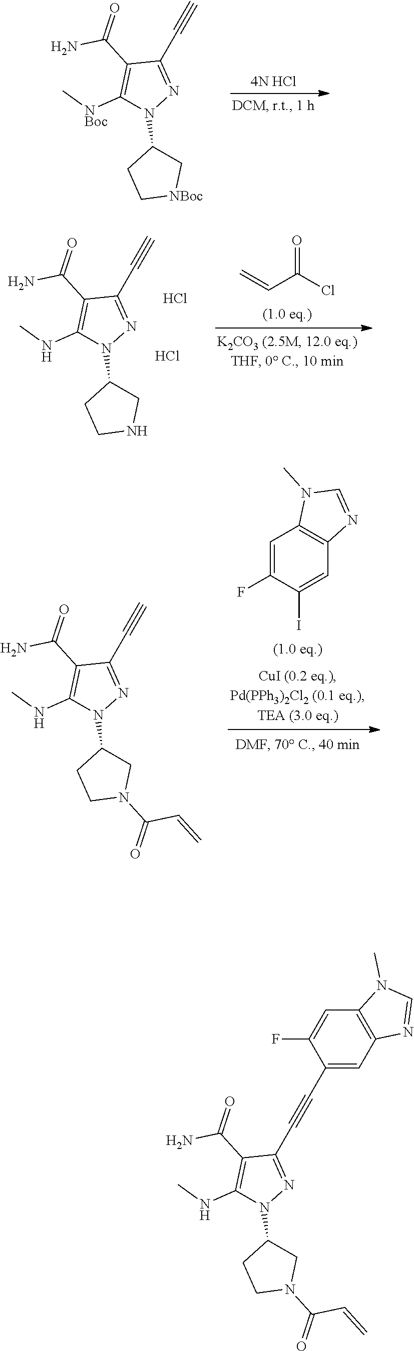

3-[2-(6-fluoro-1-methyl-1,3-benzodiazol-5-yl)ethynyl]-5-(methylamino)-1-[(3S)-1-(prop-2-enoyl)pyrrolidin-3-yl]pyrazole-4-carboxamide MS ESI calculated for C₂₂H₂₂FN₇O₂ [M+H]⁺, 436.20, found 436.20; ¹H NMR (400 MHz, DMSO-d₆) δ 8.31 (s, 1H), 7.96-7.73 (m, 1H), 7.68 (d, J=9.7 Hz, 1H), 7.48 (s, 1H), 6.80 (s, 1H), 6.68 (d, J=6.1, 4.6 Hz, 1H), 6.68-6.54 (m, 1H), 6.17-5.89 (m, 1H), 5.69-5.43 (in, 2.4 Hz, 1H), 5.25-5.08 (m, 1H), 4.10-3.99 (m, 1H), 3.92-3.83 (m, 1H), 3.84 (s, 3H), 3.82-3.71 (m, 1H), 3.73-3.50 (m, 1H), 2.96-2.73 (m, 3H), 2.40-2.35 (m, 1H), 2.29-2.10 (m, 1H).

### Example 140: 3-[2-(1-Ethyl-6,7-difluoro-1,3-benzodiazol-5-yl)ethynyl]-5-(methylamino)-1-[(3 S)-1-(prop-2-enoyl)pyrrolidin-3-yl]pyrazole-4-carboxamide

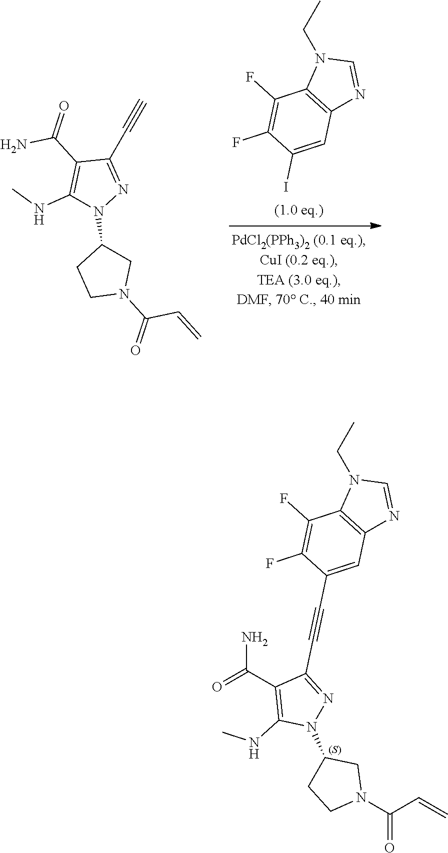

3-[2-(1-ethyl-6,7-difluoro-1,3-benzodiazol-5-yl)ethynyl]-5-(methylamino)-1-[(3S)-1-(prop-2-enoyl)pyrrolidin-3-yl]pyrazole-4-carboxamide. MS ESI calculated for C₂₃H₂₃F₂N₇O₂ [M+H]⁺, 468.20, found 468.10; ¹H NMR (300 MHz, DMSO) δ 8.44 (s, 1H), 7.85 (d, J=5.3 Hz, 1H), 7.47 (s, 1H), 6.92-6.55 (m, 3H), 6.25-6.12 (m, 1H), 5.71-5.62 (m, 1H), 5.18-5.12 (m, 1H), 4.40 (q, J=7.2 Hz, 2H), 4.12-3.60 (m, 4H), 2.97-2.92 (m, 3H), 2.42-2.24 (m, 2H), 1.47 (t, J=7.2 Hz, 3H).

### Example 141: 3-[2-(6-Chloro-1-ethyl-7-fluoro-1,3-benzodiazol-5-yl)ethynyl]-5-(methylamino)-1-[(3 S)-1-(prop-2-enoyl)pyrrolidin-3-yl]pyrazole-4-carboxamide

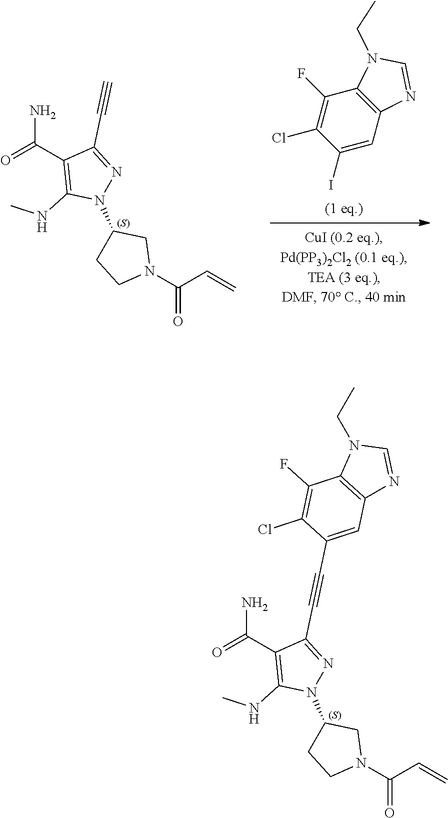

3-[2-(6-chloro-1-ethyl-7-fluoro-1,3-benzodiazol-5-yl)ethynyl]-5-(methylamino)-1-[(3S)-1-(prop-2-enoyl)pyrrolidin-3-yl]pyrazole-4-carboxamide. MS ESI calculated for C₂₃H₂₃ClFN₇O₂ [M+H]⁺, 484.20, found 484.05; ¹H NMR (400 MHz, DMSO-d₆) δ 8.46 (di, J=1.2 Hz, 1H), 7.93 (d, J=1.6 Hz, 1H), 7.49 (s, 1H), 6.85 (s, 1H), 6.71-6.55 (m, 2H), 6.17-5.96 (m, 1H), 5.70-5.46 (m, 1H), 5.25-5.08 (m, 1H), 4.39-4.12 (m, 2H), 3.92-3.50 (m, 4H), 2.96-2.64 (m, 3H), 2.41-2.21 (m, 1H), 2.34-2.26 (m, 1H), 1.45 (m, 3H).

### Example 142: 3-[2-(6-Chloro-7-fluoro-1-methyl-1,3-benzodiazol-5-yl)ethynyl]-5-(methylamino)-1-[(3 S)-1-(prop-2-enoyl)pyrrolidin-3-yl]pyrazole-4-carboxamide

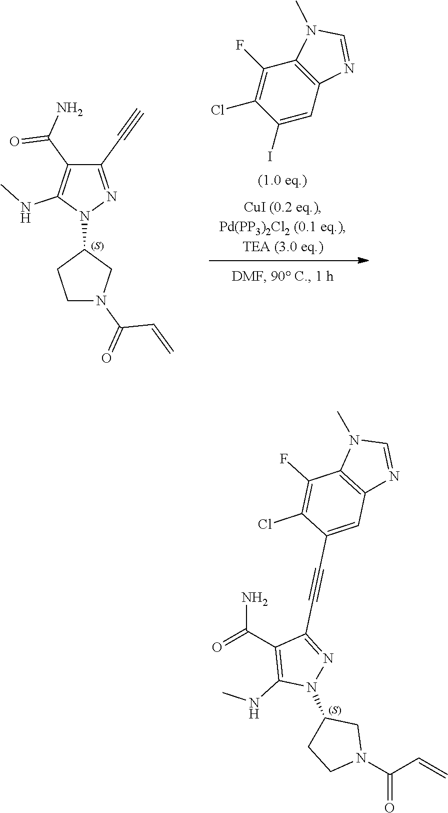

3-[2-(6-chloro-7-fluoro-1-methyl-1,3-benzodiazol-5-yl)ethynyl]-5-(methylamino)-1-[(3S)-1-(prop-2-enoyl)pyrrolidin-3-yl]pyrazole-4-carboxamide. MS ESI calculated for C₂₂H₂₁ClFN₇O₂ [M+H]⁺, 470.14, found 470.10; ¹H NMR (400 MHz, DMSO) δ 8.38 (s, 1H), 7.92 (s, 1H), 7.48 (s, 1H), 6.85 (s, 1H), 6.69-6.57 (m, 2H), 6.20-6.14 (m, 1H), 5.73-5.67 (m, 1H), 5.22-5.10 (m, 1H), 4.06-3.83 (m, 5H), 3.76-3.33 (m, 2H), 2.97 (s, 3H), 2.43-2.38 (m, 1H), 2.32-2.30 (m, 1H).

### Example 143: 3-[2-(1-Ethyl-6-fluoro-1,3-benzodiazol-5-yl)ethynyl]-5-(methylamino)-1-[(3 S)-1-(prop-2-enoyl)pyrrolidin-3-yl]pyrazole-4-carboxamide

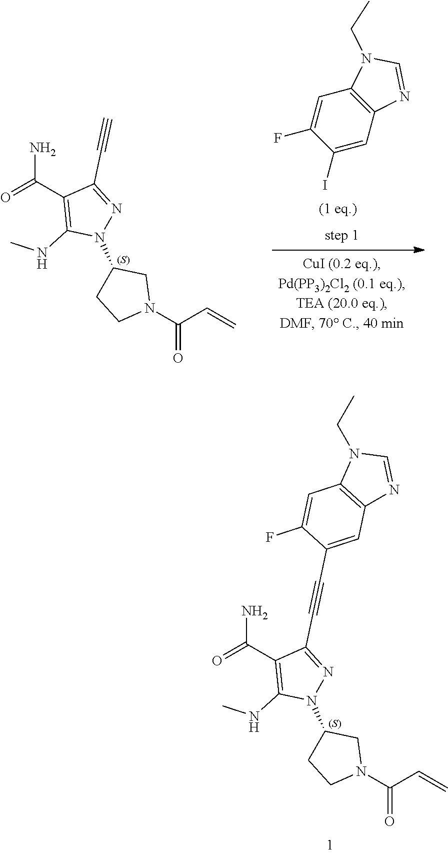

3-[2-(1-ethyl-6-fluoro-1,3-benzodiazol-5-yl)ethynyl]-5-(methylamino)-1-[(3S)-1-(prop-2-enoyl)pyrrolidin-3-yl]pyrazole-4-carboxamide. MS ESI calculated for C₂₃H₂₄FN₇O₂ [M+H]⁺, 450.20, found 450.15; ¹H NMR (300 MHz, DMSO-d₆) δ 8.41 (s, 1H), 7.97-7.77 (m, 1H), 7.76-7.56 (m, 1H), 7.49 (s, 1H), 6.81 (s, 1H), 6.70 (s, 1H), 6.74-6.54 (m, 1H), 6.25-6.11 (m, 1H), 5.71-5.43 (m, 1H), 5.18-4.96 (m, 1H), 4.29 (q, J=7.3 Hz, 2H), 4.12-3.51 (m, 4H), 2.97-2.69 (m, 3H), 2.45-2.27 (m, 2H), 1.42-1.23 (m, 3H).

### Example 144: 3-[2-(6-Chloro-1-methyl-1,3-benzodiazol-5-yl)ethynyl]-5-(methylamino)-1-[(3 S)-1-(prop-2-enoyl)pyrrolidin-3-yl]pyrazole-4-carboxamide

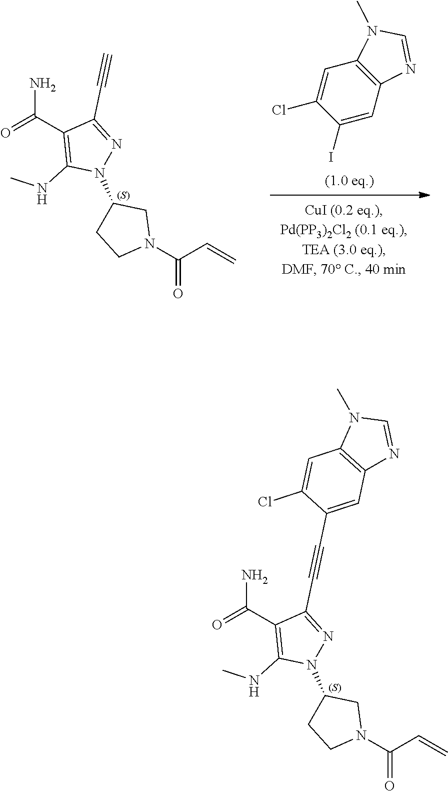

3-[2-(6-chloro-1-methyl-1,3-benzodiazol-5-yl)ethynyl]-5-(methylamino)-1-[(3S)-1-(prop-2-enoyl)pyrrolidin-3-yl]pyrazole-4-carboxamide. MS ESI calculated for C₂₂H₂₂ClN₇O₂ [M+H]⁺, 452.16, found 452.15; ¹H NMR (300 MHz, DMSO) δ 8.36 (s, 1H), 8.04 (s, 1H), 7.95 (s, 1H), 7.51 (s, 1H), 6.88 (s, 1H), 6.78-6.54 (m, 2H), 6.18-6.13 (m, 1H), 5.70-5.65 (m, 1H), 5.18-5.13 (m, 1H), 3.87-3.81 (m, 4H), 3.85-3.49 (m, 3H), 2.97-2.93 (m, 3H), 2.38-2.32 (m, 2H).

### Example 145: 3-[2-(6,7-Difluoro-1-methyl-1,3-benzodiazol-5-yl)ethynyl]-5-(methylamino)-1-[(3 S)-1-(prop-2-enoyl)pyrrolidin-3-yl]pyrazole-4-carboxamide

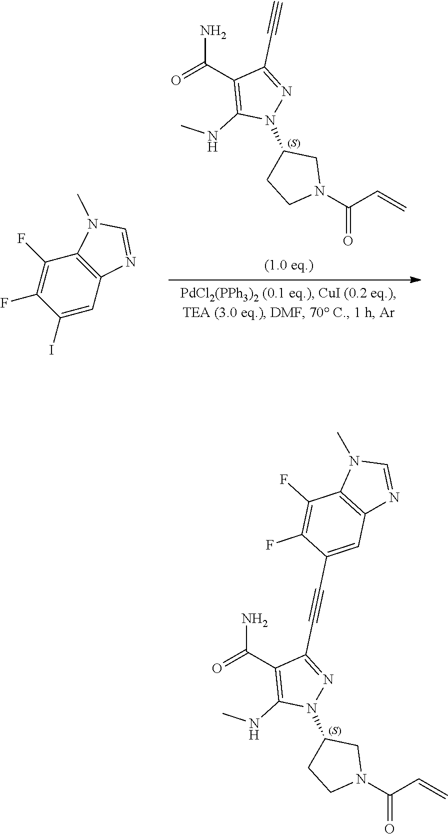

3-[2-(6,7-difluoro-1-methyl-1,3-benzodiazol-5-yl)ethynyl]-5-(methylamino)-1-[(3S)-1-(prop-2-enoyl)pyrrolidin-3-yl]pyrazole-4-carboxamide. MS ESI calculated for C₂₂H₂₁F₂N₇O₂ [M+H]⁺, 454.17, found 454.15; ¹H NMR (300 MHz, DMSO-d₆) δ 8.35 (s, 1H), 7.83 (d, J=5.2 Hz, 1H), 7.47 (s, 1H), 6.71-6.57 (m, 3H), 6.21-6.14 (m, 1H), 5.74-5.67 (m, 1H), 5.22-5.13 (m, 1H), 4.07 (s, 3H), 3.91-3.58 (m, 4H), 2.97-2.92 (m, 3H), 2.52-2.28 (in, 2H).

### Example 147: 3-[2-(6-Chloro-1-cyclopropyl-7-fluoro-1,3-benzodiazol-5-yl)ethynyl]-1-[(3 S,5R)-5-(methoxymethyl)-1-(prop-2-enoyl)pyrrolidin-3-yl]-5-(methylamino)pyrazole-4-carboxamide

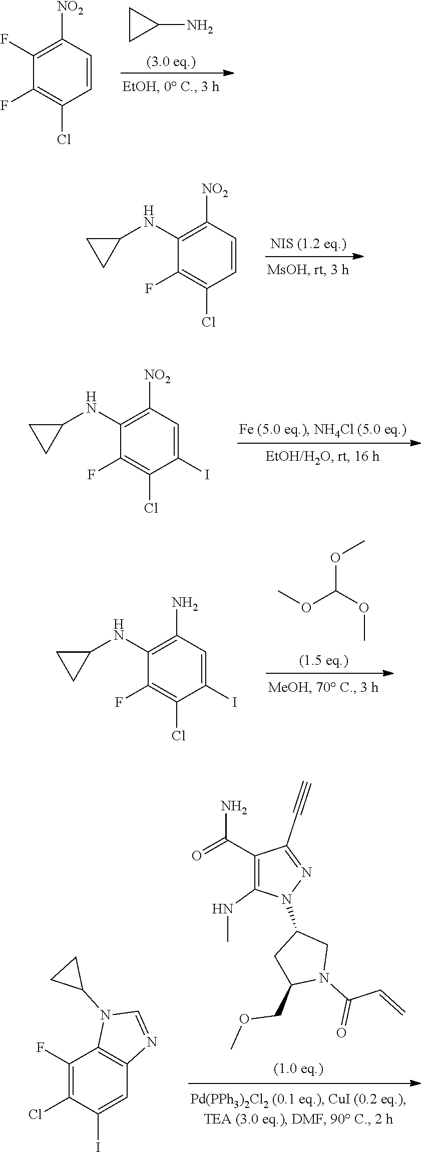

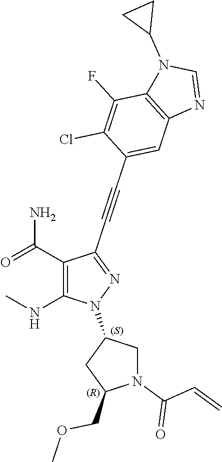

3-[2-(6-chloro-1-cyclopropyl-7-fluoro-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide. MS ESI calculated for C₂₆H₂₇ClFN₇O₃[M+H]⁺, 540.18, found 540.30; ¹H NMR (300 MHz, CDCl₃) δ 8.01 (brs, 1H), 7.12 (brs, 1H), 6.83 (brs, 1H), 6.67-6.33 (m, 2H), 5.77-5.71 (m, 1H), 5.65-5.21 (m, 2H), 4.66-4.38 (m, 1H), 4.26-3.83 (m, 2H), 3.73-3.28 (m, 6H), 3.05-3.01 (m, 3H), 2.89-2.57 (m, 1H), 2.36-2.31 (m, 1H), 1.44-0.97 (m, 4H).

### Example 148: 3-[2-(6-Fluoro-1-methyl-1,3-benzodiazol-5-yl)ethynyl]-1-[(3S,5R)-5-(fluoromethyl)-1-(prop-2-enoyl)pyrrolidin-3-yl]-5-(methylamino)pyrazole-4-carboxamide

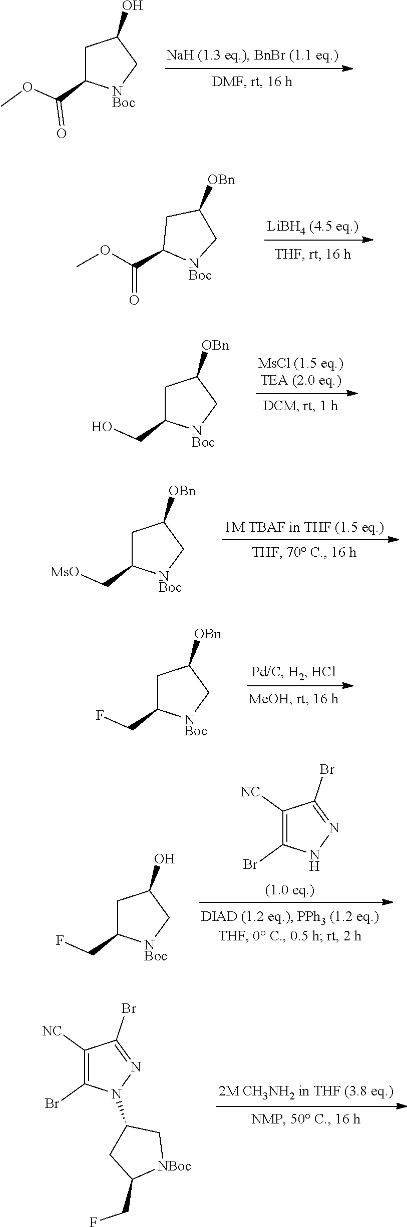

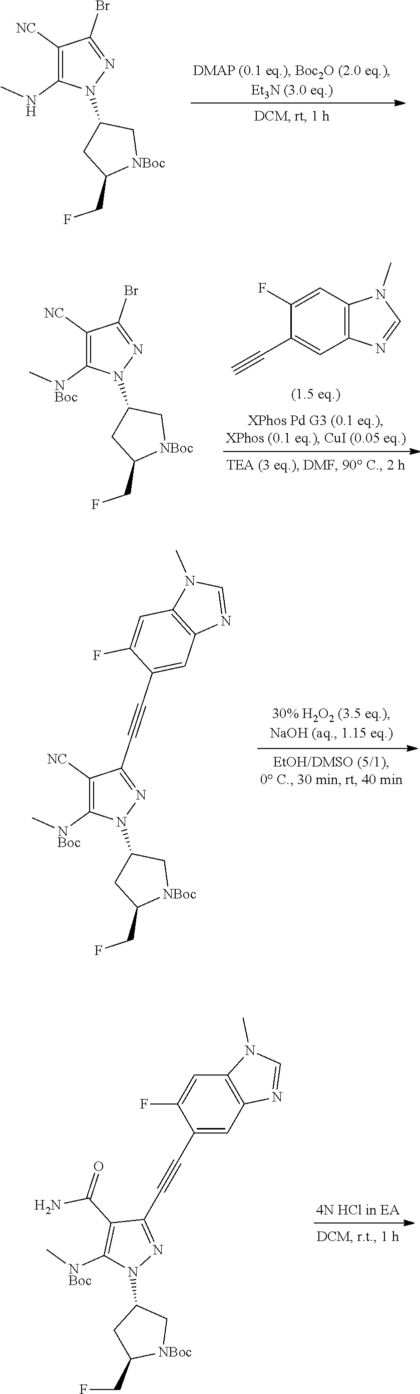 [SMILES: CNc1c(C#N)c(Br)nn1[C@H]1C[C@H](CF)N(C(=O)OC(C)(C)C)C1 ; CN(C(=O)OC(C)(C)C)c1c(C#N)c(Br)nn1[C@H]1C[C@H](CF)N(C(=O)OC(C)(C)C)C1 ; C#Cc1cc2ncn(C)c2cc1F ; CN(C(=O)OC(C)(C)C)c1c(C#N)c(C#Cc2cc3ncn(C)c3cc2F)nn1[C@H]1C[C@H](CF)N(C(=O)OC(C)(C)C)C1 ; CN(C(=O)OC(C)(C)C)c1c(C(N)=O)c(C#Cc2cc3ncn(C)c3cc2F)nn1[C@H]1C[C@H](CF)N(C(=O)OC(C)(C)C)C1]

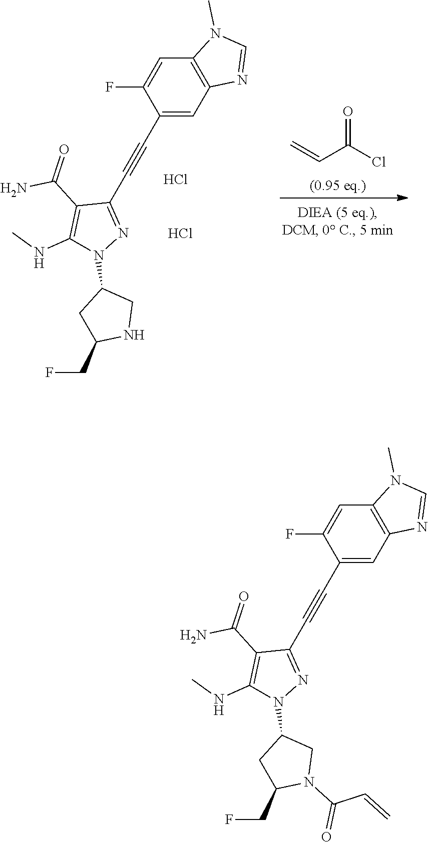

3-[2-(6-fluoro-1-methyl-1,3-benzodiazol-5-yl)ethynyl]-1-[(3S,5R)-5-(fluoromethyl)-1-(prop-2-enoyl)pyrrolidin-3-yl]-5-(methylamino)pyrazole-4-carboxamide. MS ESI calculated for C₂₃H₂₃F₂N₇O₂[M+H]⁺, 468.19, found 468.25; ¹H NMR (300 MHz, CDCl₃) δ 7.98 (d, J=6.3 Hz, 1H), 7.91 (s, 1H), 7.14 (d, J=9.0 Hz, 2H), 6.80-6.78 (m, 1H), 6.43 (d, J=2.1 Hz, 1H), 6.41 (s, 1H), 5.76-5.72 (m, 1H), 5.43-5.40 (m, 1H), 5.37-5.29 (m, 1H), 5.05-4.85 (m, 1H), 4.68-4.40 (m, 2H), 4.17-4.04 (m, 2H), 3.84 (s, 3H), 3.03 (d, J=6.0 Hz, 3H), 2.89-2.78 (m, 1H), 2.43-2.46 (m, 1H).

### Example 149: 1-[(3S,5R)-5-(Methoxymethyl)-1-(prop-2-enoyl)pyrrolidin-3-yl]-5-(methylamino)-3-[2-(4,6,7-trifluoro-1,2-dimethyl-1,3-benzodiazol-5-yl)ethynyl]pyrazole-4-carboxamide

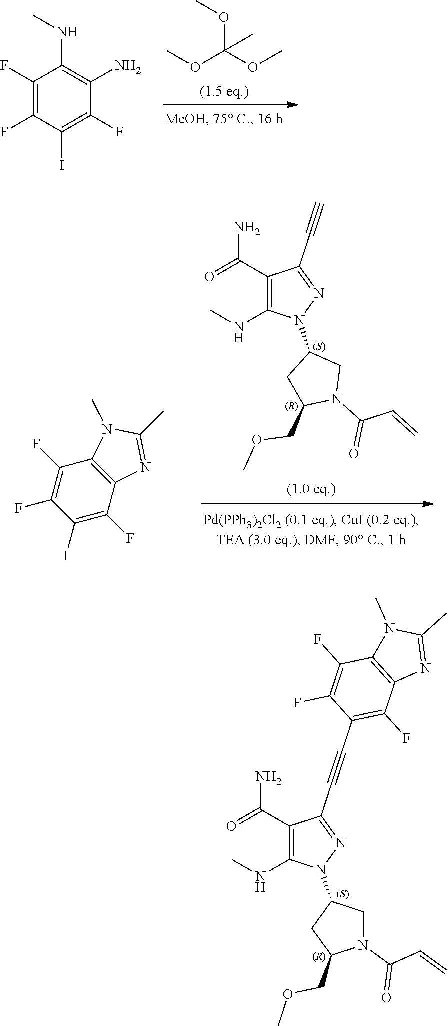

1-[(3S,5R)-5-(methoxymethyl)-1-(prop-2-enoyl)pyrrolidin-3-yl]-5-(methylamino)-3-[2-(4,6,7-trifluoro-1,2-dimethyl-1,3-benzodiazol-5-yl)ethynyl]pyrazole-4-carboxamide. MS ESI calculated for C₂₅H₂₆F₃N₇O₃ [M+H]⁺, 530.20, found 530.20; ¹H NMR (400 MHz, CDCl₃) δ 7.05-6.98 (m, 1H), 6.89-6.82 (m, 1H), 6.49-6.41 (m, 2H), 5.78-5.72 (m, 1H), 5.56-5.32 (m, 2H), 4.58-4.32 (m, 1H), 4.18-4.01 (m, 2H), 3.98-3.91 (m, 4H), 3.54-3.45 (m, 1H), 3.38 (s, 3H), 3.09-2.99 (m, 3H), 2.79-2.71 (m, 1H), 2.61 (s, 3H), 2.39-2.31 (m, 1H).

### Example 150: 3-[2-(1-Cyclopropyl-6-fluoro-2-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

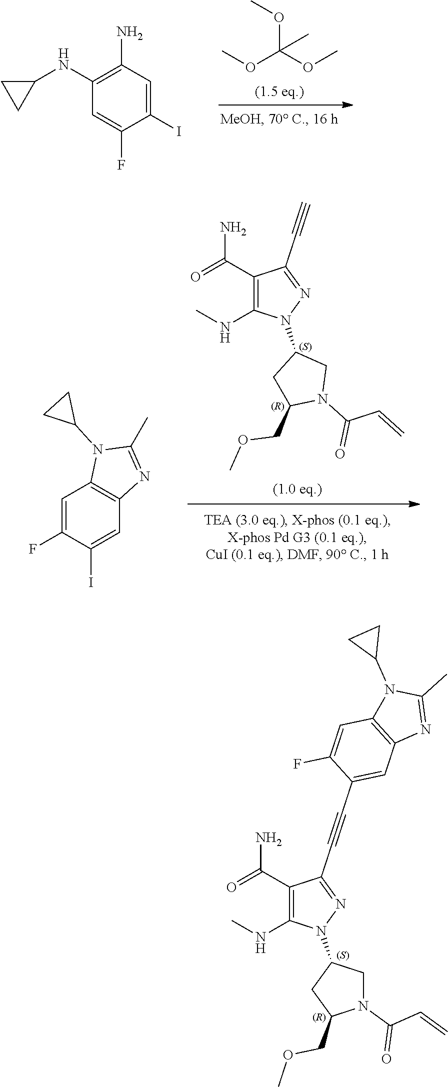

3-[2-(1-cyclopropyl-6-fluoro-2-methyl-1,3-benzodiazol-5-yl) ethynyl]-1-[(3S,5R)-5-(methoxymethyl)-1-(prop-2-enoyl)pyrrolidin-3-yl]-5-(methylamino)pyrazole-4-carboxamide. MS ESI calculated for C₂₇H₃₀FN₇O₃[M+H]⁺, 520.25, found 520.30; ¹H NMR (400 MHz, CDCl₃) δ 7.87 (s, 1H), 7.16 (s, 1H), 6.83 (d, J=6.0 Hz, 1H), 6.58-6.35 (m, 2H), 5.72-5.68 (m, 1H), 5.58-5.22 (m, 2H), 4.56 (d, J=8.9 Hz, 1H), 4.12-4.03 (m, 2H), 3.91-3.85 (m, 1H), 3.54-3.41 (m, 1H), 3.38 (d, J=4.4 Hz, 3H), 3.20-3.15 (m, 1H), 3.05-3.01 (m, 3H), 2.80-2.48 (m, 3H), 2.31-2.26 (m, 1H), 1.29-1.27 (m, 2H), 1.11-1.02 (m, 2H).

### Example 151: 3-[2-(1-Cyclopropyl-6,7-difluoro-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

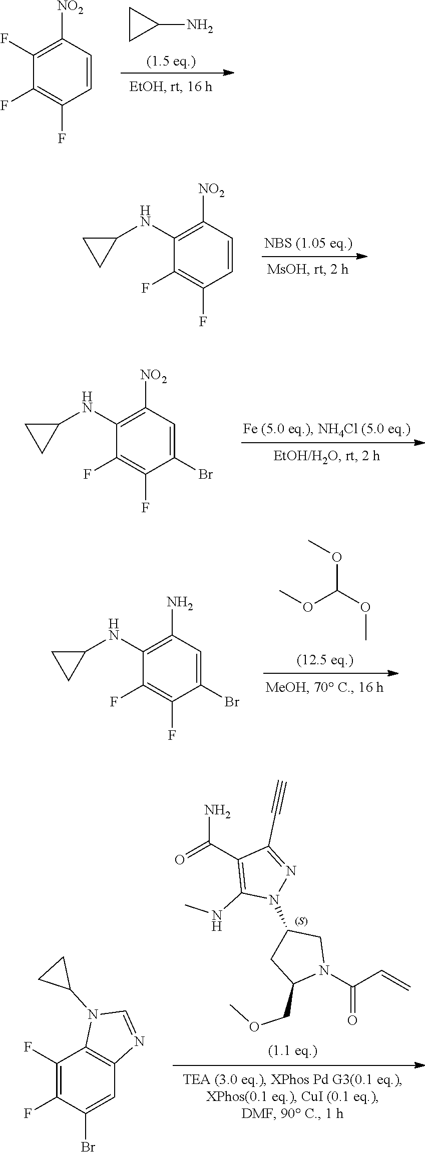

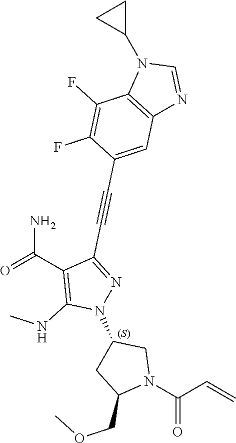

3-[2-(1-cyclopropyl-6,7-difluoro-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide. MS ESI calculated for C₂₆H₂₇F₂N₇O₃[M+H]⁺, 524.21, found 524.30; ¹H NMR (400 MHz, CDCl₃) δ 7.94 (s, 1H), 7.75 (d, J=5.0 Hz, 1H), 7.05-6.95 (m, 1H), 6.85-6.82 (m, 1H), 6.55-6.39 (m, 2H), 5.72-5.68 (m, 1H), 5.53-5.45 (m, 1H), 5.41-5.26 (m, 1H), 4.58 (d, J=9.1 Hz, 1H), 4.16-3.87 (m, 3H), 3.73-3.52 (m, 1H), 3.55-3.42 (m, 1H), 3.39 (d, J=4.6 Hz, 3H), 3.05 (d, J=15.5 Hz, 3H), 2.83-2.62 (m, 1H), 2.45-2.26 (m, 1H), 1.28-1.08 (m, 4H).

### Example 152: 1-[(3S,5R)-5-(Methoxymethyl)-1-(prop-2-enoyl)pyrrolidin-3-yl]-5-(methylamino)-3-[2-(4,6,7-trifluoro-1-methyl-1,3-benzodiazol-5-yl)ethynyl]pyrazole-4-carboxamide

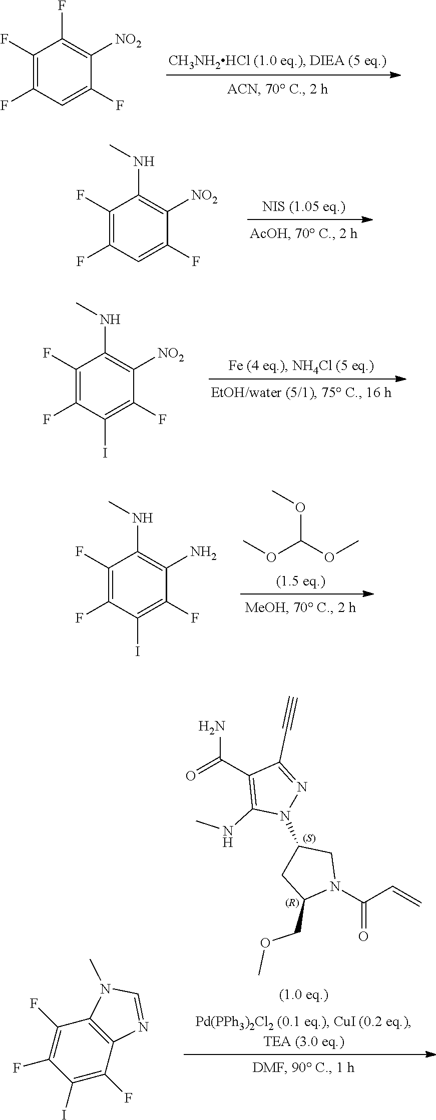

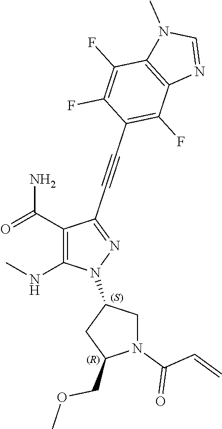

1-[(3S,5R)-5-(methoxymethyl)-1-(prop-2-enoyl)pyrrolidin-3-yl]-5-(methylamino)-3-[2-(4,6,7-trifluoro-1-methyl-1,3-benzodiazol-5-yl)ethynyl]pyrazole-4-carboxamide. MS ESI calculated for C₂₄H₂₄F₃N₇O₃[M+H]⁺, 516.19, found 516.10; ¹H NMR (400 MHz, CDCl₃) δ 7.86 (s, 1H), 6.99 (s, 1H), 6.57-6.39 (m, 2H), 5.74-5.71 (m, 1H), 5.54-5.42 (m, 2H), 4.59-4.56 (m, 1H), 4.15-4.02 (m, 6H), 3.53-3.44 (m, 1H), 3.39 (d, J=4 Hz, 3H), 3.05 (d, J=12 Hz, 3H), 2.76-2.68 (m, 1H), 2.42-2.30 (m, 1H).

### Example 153: 3-[2-(1-Cyclopropyl-4,6,7-trifluoro-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

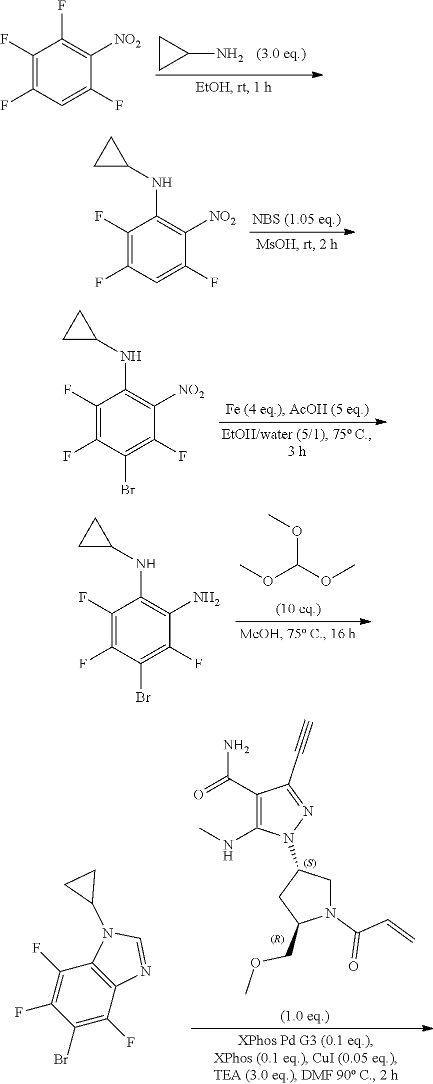

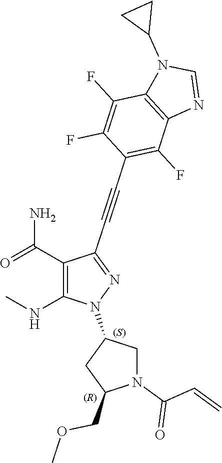

3-[2-(1-cyclopropyl-4,6,7-trifluoro-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide. MS ESI calculated for C₂₆H₂₆F₃N₇O₃[M+H]⁺, 542.20, found 542.20; ¹H NMR (400 MHz, CDCl₃) δ 7.92 (s, 1H), 6.99 (s, 1H), 6.50-6.41 (m, 2H), 5.72-5.69 (m, 1H), 5.55-5.48 (m, 1H), 5.48-5.24 (m, 1H), 4.61-4.39 (m, 1H), 4.08-4.01 (m, 2H), 3.99-3.39 (m, 1H), 3.62-3.60 (m, 1H), 3.54-3.42 (m, 1H), 3.39 (d, J=5.0 Hz, 3H), 3.05 (d, J=15.8 Hz, 3H), 2.81-2.63 (m, 1H), 2.39-2.26 (m, 1H), 1.32-1.23 (m, 2H), 1.22-1.12 (m, 2H).

### Example 154: 1-[(3S,5R)-5-[(Difluoromethoxy)methyl]-1-(prop-2-enoyl)pyrrolidin-3-yl]-3-[2-(1-ethyl-6,7-difluoro-1,3-benzodiazol-5-yl)ethynyl]-5-(methylamino)pyrazole-4-carboxamide

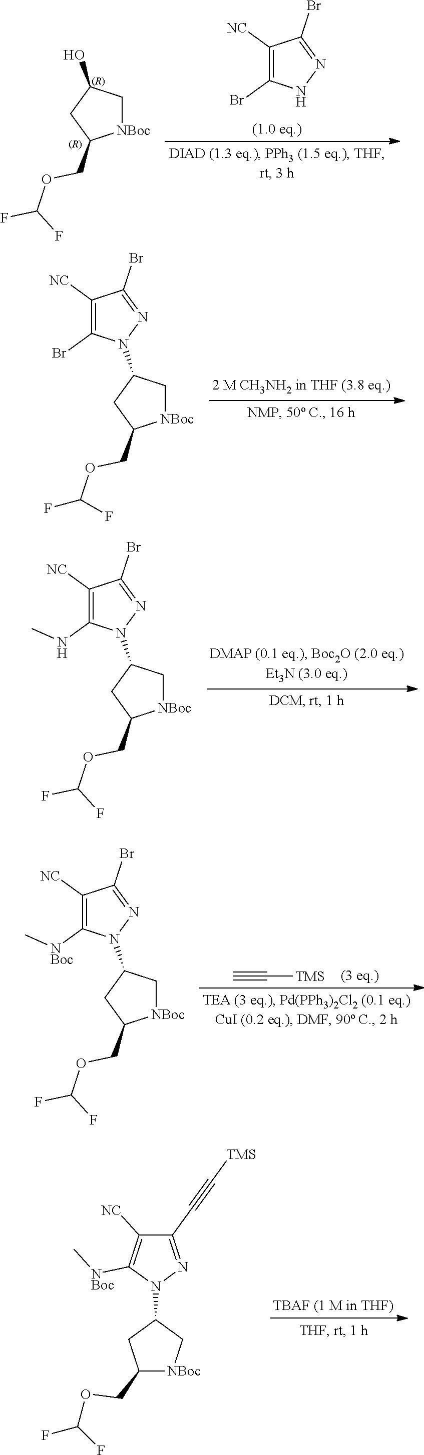

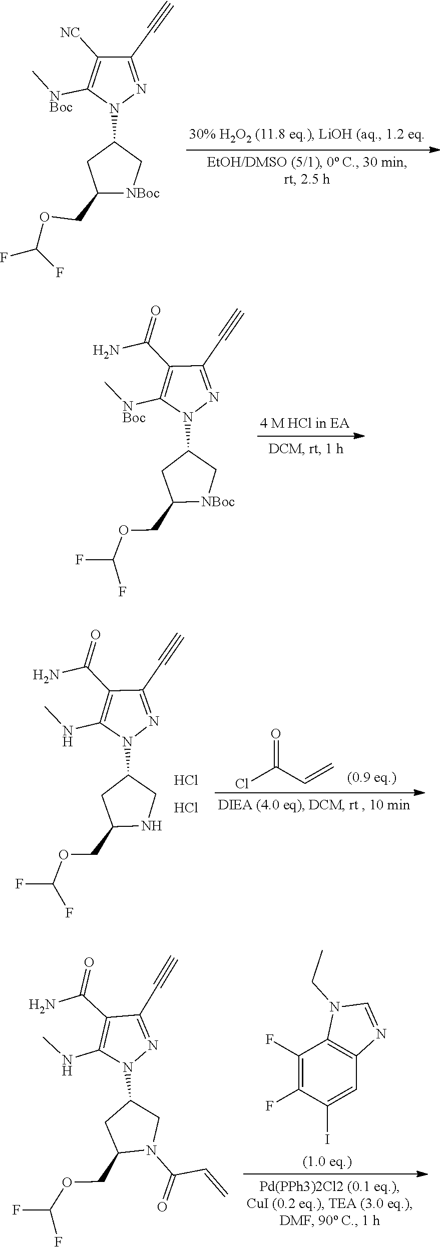

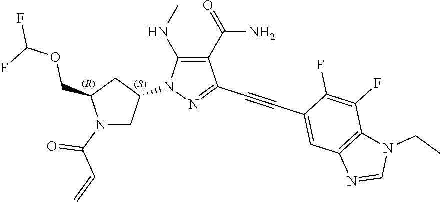

1-[(3S,5R)-5-[(difluoromethoxy)methyl]-1-(prop-2-enoyl)pyrrolidin-3-yl]-3-[2-(i-ethyl-6,7-difluoro-1,3-benzodiazol-5-yl)ethynyl]-5-(methylamino)pyrazole-4-carboxamide. MS ESI calculated for C₂₅H₂₅F₄N₇O₃[M+H]+, 548.20, found 548.15; ¹H NMR (400 MHz, CDCl₃) δ 7.91 (s, 1H), 7.76-7.45 (m, 1H), 7.02 (s, 1H), 6.82-6.43 (m, 1H), 6.52-6.34 (m, 3H), 5.80-5.70 (m, 1H), 5.51-5.28 (m, 2H), 4.68-4.61 (m, 1H), 4.46-4.33 (m, 3H), 4.09-3.98 (m, 2H), 3.94-3.74 (m, 1H), 3.04-2.96 (m, 3H), 2.83-2.63 (m, 1H), 2.41-2.32 (m, 1H), 1.57-1.32 (m, 3H).

### Example 155: 3-[2-(1-Cyclopropyl-1,3-benzodiazol-5-yl)ethynyl]-1-[(3S,5R)-5-[(difluoromethoxy)methyl]-1-(prop-2-enoyl)pyrrolidin-3-yl]-5-(methylamino)pyrazole-4-carboxamide

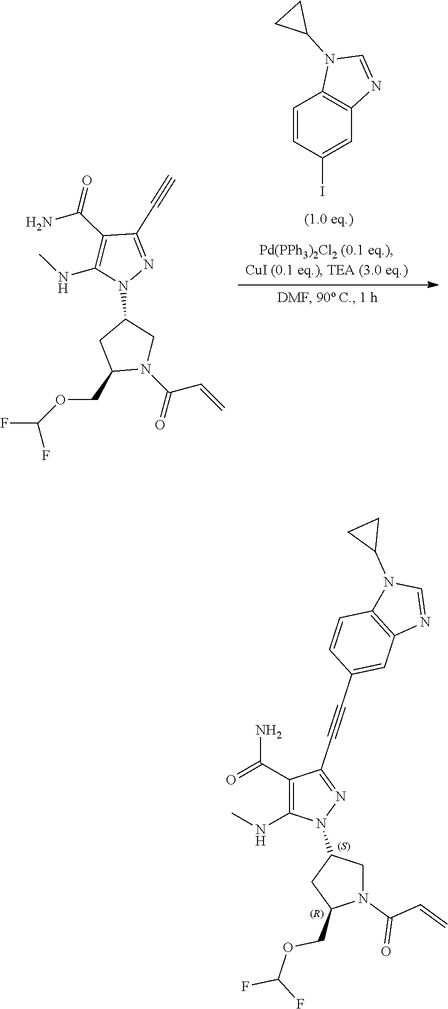

3-[2-(1-cyclopropyl-1,3-benzodiazol-5-yl)ethynyl]-1-[(3S,5R)-5-[(difluoromethoxy)methyl]-1-(prop-2-enoyl)pyrrolidin-3-yl]-5-(methylamino)pyrazole-4-carboxamide. MS ESI calculated for C₂₆H₂₇F₂N₇O₃ [M+H]⁺, 524.21, found 524.20; ¹H NMR (400 MHz, CDCL₃) δ 8.04 (d, J=96.4 Hz, 2H), 7.48 (d, J=8.0 Hz, 1H), 6.94-6.92 (m, 1H), 6.77 (d, J=6.2 Hz, 1H), 6.56-5.97 (m, 3H), 5.79-5.69 (m, 1H), 5.40-5.38 (m, 1H), 4.68-4.60 (m, 1H), 4.41 (dd, J=10.3, 3.1 Hz, 1H), 4.13-4.12 (m, 1H), 4.04-4.01 (m, 1H), 3.93-3.92 (m, 1H), 3.40 (s, 1H), 3.03-2.98 (m, 3H), 2.83-2.37 (m, 1H), 2.41-2.31 (m, 1H), 1.20-1.18 (m, 2H), 1.07-1.05 (m, 2H).

### Example 156: 1-((3S,5R)-1-Acryloyl-5-((difluoromethoxy)methyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide

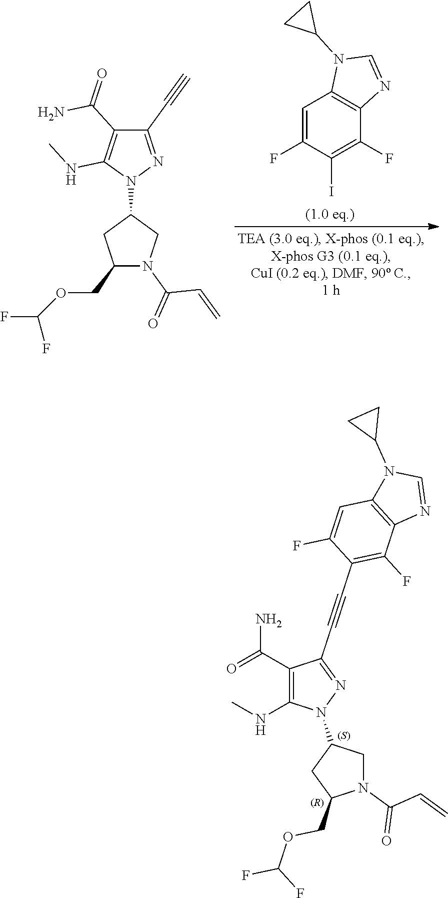

1-((3S,5R)-1-acryloyl-5-((difluoromethoxy)methyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide. MS ESI calculated for C₂₆H₂₅F₄N₇O₃[M+H]⁺, 560.20; found 560.15; ¹H NMR (400 MHz, CDCl₃) δ 7.95 (s, 1H), 7.27 (s, 1H), 7.16 (d, J=8.2 Hz, 1H), 7.05 (s, 1H), 6.88-6.00 (m, 3H), 5.74-5.71 (m, 1H), 5.56-5.01 (m, 2H), 4.63 (d, J=9.2 Hz, 1H), 4.42 (d, J=10.3, 3.2 Hz, 1H), 4.14 (d, J=9.8, 8.0 Hz, 1H), 4.04 (d, J=9.9, 8.1 Hz, 1H), 3.94 (dd, J=10.4, 2.3 Hz, 1H), 3.38-3.35 (m, 1H), 3.03 (s, 3H), 2.84-2.81 (m, 1H), 2.37-2.35 (m, 1H), 1.28-1.14 (m, 2H), 1.11-1.03 (m, 2H).

### Example 157: 3-[2-(1-Ethyl-4,6,7-trifluoro-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

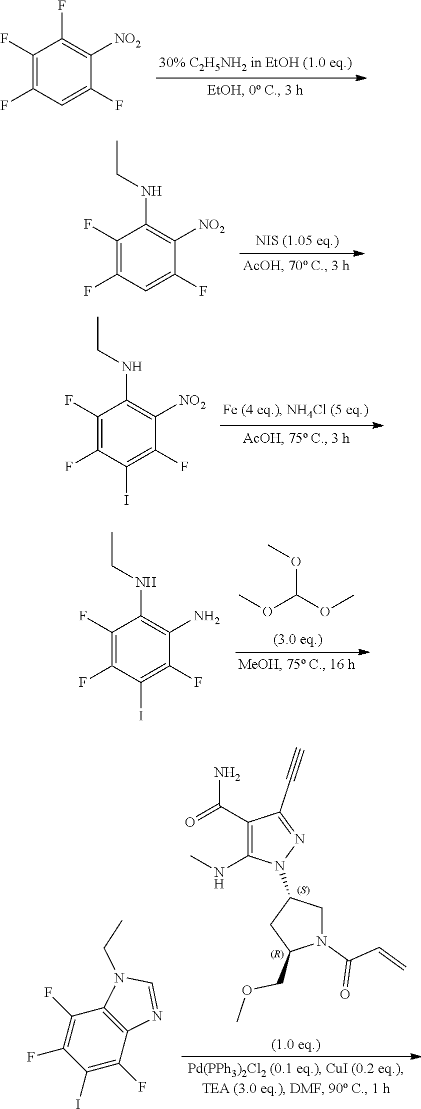

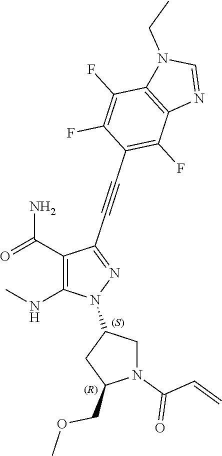

3-[2-(1-ethyl-4,6,7-trifluoro-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide. MS ESI calculated for C₂₅H₂₆F₃N₇O₃ [M+H]⁺, 530.20, found 530.25; ¹H NMR (400 MHz, CDCl₃) δ 7.92 (s, 1H), 6.99 (s, 1H), 6.63-6.32 (m, 2H), 5.80-5.70 (m, 1H), 5.57-5.25 (m, 2H), 4.57 (d, J=9.0 Hz, 1H), 4.41 (q, J=7.2 Hz, 2H), 4.12 (d, J=8.7 Hz, 1H), 4.07-3.97 (m, 1H), 3.93-3.90 (m, 1H), 3.56-3.41 (m, 1H), 3.39 (d, J=5.1 Hz, 3H), 3.05 (d, J=15.7 Hz, 3H), 2.77-2.66 (m, 1H), 2.34-2.29 (m, 1H), 1.60 (t, J=7.3 Hz, 3H).

### Example 158: 3-[2-(1-Cyclopropyl-6,7-difluoro-1,3-benzodiazol-5-yl)ethynyl]-1-[(3S,5S)-5-methyl-1-(prop-2-enoyl)pyrrolidin-3-yl]-5-(methylamino)pyrazole-4-carboxamide

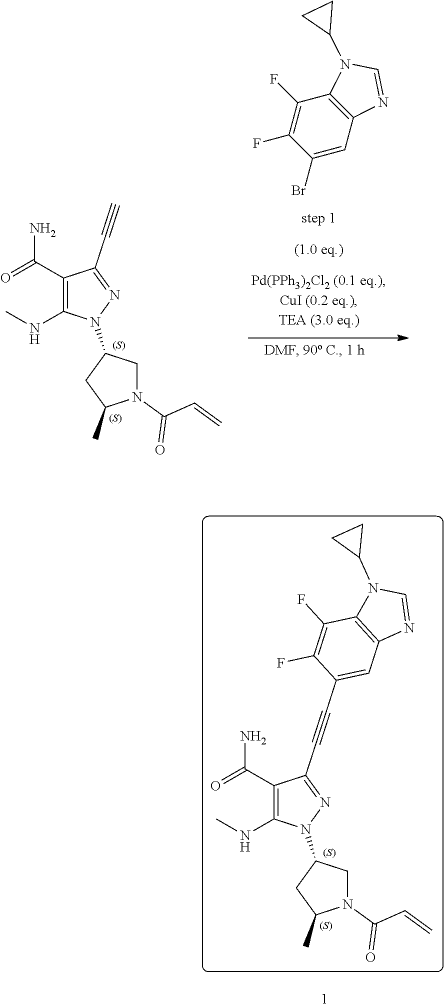

3-[2-(1-cyclopropyl-6,7-difluoro-1,3-benzodiazol-5-yl)ethynyl]-1-[(3S,5S)-5-methyl-1-(prop-2-enoyl)pyrrolidin-3-yl]-5-(methylamino)pyrazole-4-carboxamide. MS ESI calculated for C₂₅H₂₅F₂N₇O₂[M+H]+, 494.50, found 494.20; ¹H NMR (400 MHz, CDCl₃) δ 7.95 (s, 1H), 7.74 (s, 1H), 7.02 (s, 1H), 6.59-6.40 (m, 1H), 6.40-6.29 (m, 1H), 5.80-5.59 (m, 1H), 5.40 (s, 1H), 5.13-4.96 (m, 1H), 4.52-4.32 (0, 1H), 4.16-3.93 (m, 2H), 3.61-3.46 (m, 1H), 3.01-2.96 (m, 3H), 2.87-2.66 (m, 1H), 2.07-1.85 (D, 1H), 1.35-1.27 (m, 3H), 1.26-1.21 (m, 2H), 1.18-1.12 (n, 2H).

### Example 159: 3-[2-(1-Cyclopropyl-4,6-difluoro-1,3-benzodiazol-5-yl)ethynyl]-1-[(3 S,5S)-5-methyl-1-(prop-2-enoyl)pyrrolidin-3-yl]-5-(methylamino)pyrazole-4-carboxamide

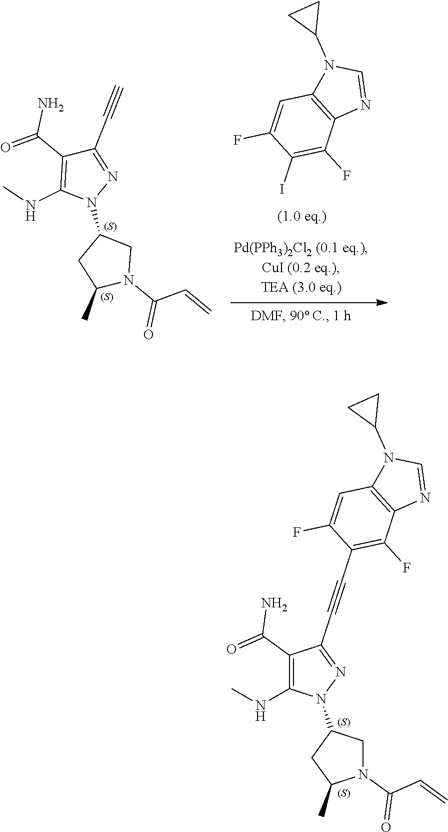

3-[2-(1-cyclopropyl-4,6-difluoro-1,3-benzodiazol-5-yl)ethynyl]-1-[(3S,5S)-5-methyl-1-(prop-2-enoyl)pyrrolidin-3-yl]-5-(methylamino)pyrazole-4-carboxamide. MS ESI calculated for C₂₅H₂₅F₂N₇O₂[M+H]+, 494.20, found 494.25; ¹H NMR (400 MHz, CDCl₃) δ 7.97 (s, 1H), 7.17 (d, J=4 Hz, 1H), 7.09 (s, 1H), 6.58-6.40 (m, 2H), 5.76-5.69 (m, 1H), 5.43 (s, 1H), 5.18-5.10 (m, 1H), 4.58-4.49 (m, 1H), 4.15-4.00 (m, 2H), 3.43-3.35 (m, 1H), 3.07-3.02 (m, 3H), 2.87-2.79 (m, 1H), 2.80-2.03 (m, 1H), 1.37 (d, J=4 Hz, 3H), 1.28-1.21 (Dm, 2H), 1.11-1.06 (m, 2H).

### Example 161: 1-((3S,5R)-1-Acryloyl-5-((difluoromethoxy)methyl)pyrrolidin-3-yl)-3-((1-cyclopropyl-6-fluoro-1H-benzo[d]imidazol-5-yl)ethynyl)-5-(methylamino)-1H-pyrazole-4-carboxamide

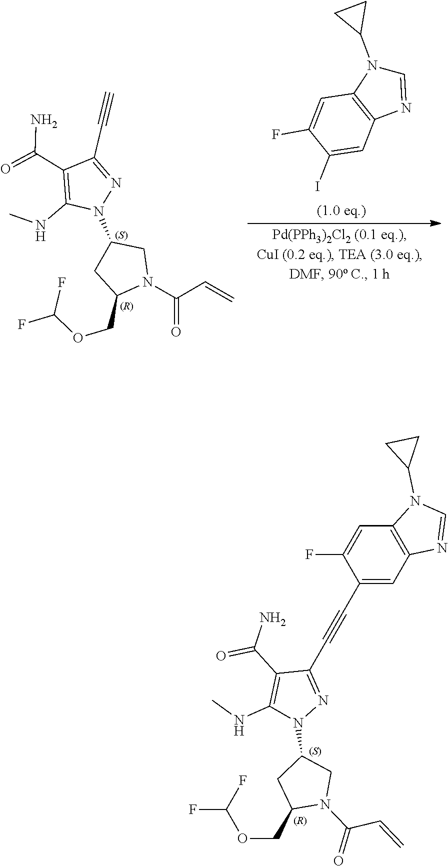

1-((3S,5R)-1-acryloyl-5-((difluoromethoxy)methyl)pyrrolidin-3-yl)-3-((1-cyclopropyl-6-fluoro-1H-benzo[d]imidazol-5-yl)ethynyl)-5-(methylamino)-1H-pyrazole-4-carboxamide. MS ESI calculated for C₂₆H₂₆F₃N₇O₃[M+H]+, 542.20, found 542.20; ¹H NMR (400 MHz, CDCl₃) δ 7.99 (s, 2H), 7.33 (d, J=9.1 Hz, 1H), 7.13 (s, 1H), 6.83 (d, J=6.3 Hz, 1H), 6.53-6.40 (m, 2H), 6.23 (s, 1H), 5.82-5.71 (m, 1H), 5.57-5.33 (m, 2H), 4.66 (d, J=9.1 Hz, 1H), 4.44 (dd, J=10.4, 3.1 Hz, 1H), 4.24-3.81 (m, 3H), 3.39-3.34 (m, 1H), 3.05 (d, J=5.8 Hz, 3H), 2.85 (dt, J=12.7, 9.6 Hz, 1H), 2.38 (dd, J=13.1, 7.2 Hz, 1H), 1.22 (q, J=6.5, 5.9 Hz, 2H), 1.09-1.01 (in, 2H).

### Example 162: 3-[2-(6-Chloro-1-cyclopropyl-1,3-benzodiazol-5-yl)ethynyl]-1-[(3S,5R)-5-(difluoromethyl)-1-(prop-2-enoyl)pyrrolidin-3-yl]-5-(methylamino)pyrazole-4-carboxamidee

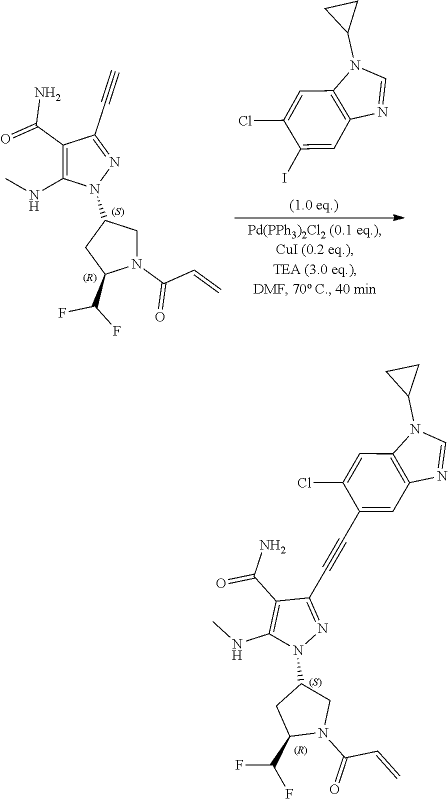

3-[2-(6-chloro-1-cyclopropyl-1,3-benzodiazol-5-yl)ethynyl]-1-[(3S,5R)-5-(difluoromethyl)-1-(prop-2-enoyl)pyrrolidin-3-yl]-5-(methylamino)pyrazole-4-carboxamide. MS ESI calculated for C₂₅H₂₄ClF₂N₇O₂ [M+H]⁺, 528.16, found 528.20; ¹H NMR (400 MHz, CDCl₃) δ 8.06 (d, J=22.6 Hz, 2H), 7.68 (s, 1H), 7.20 (s, 1H), 6.81 (s, 1H), 6.53-6.37 (m, 2H), 6.32-6.28 (m, 1H), 5.80-5.75 (m, 1H), 5.41-5.28 (m, 2H), 4.70-4.65 (m, 1H), 4.20-4.15 (m, 1H), 4.06-4.02 (m, 1H), 3.40-3.32 (m, 1H), 3.05 (s, 3H), 2.82-2.69 (m, 1H), 2.64-2.58 (m, 1H), 1.30-1.19 (m, 2H), 1.15-1.05 (m, 2H).

### Example 163: 3-[2-(1-Cyclopropyl-4,6-difluoro-1,3-benzodiazol-5-yl)ethynyl]-1-[(3S,5R)-5-(difluoromethyl)-1-(prop-2-enoyl)pyrrolidin-3-yl]-5-(methylamino)pyrazole-4-carboxamide

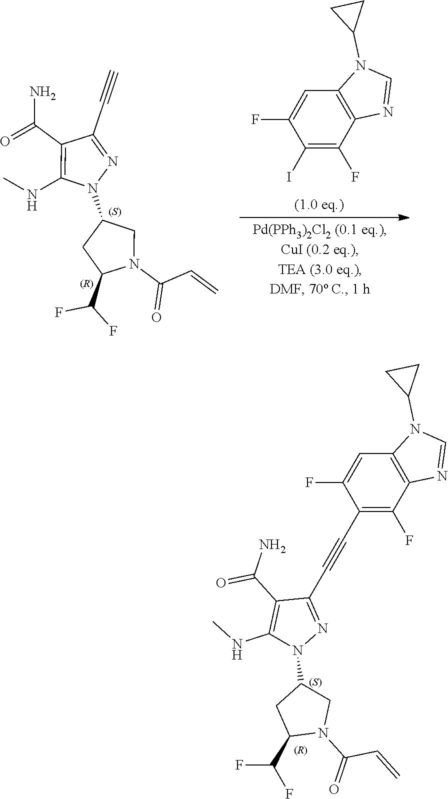

3-[2-(1-cyclopropyl-4,6-difluoro-1,3-benzodiazol-5-yl)ethynyl]-1-[(3S,5R)-5-(difluoromethyl)-1-(prop-2-enoyl)pyrrolidin-3-yl]-5-(methylamino)pyrazole-4-carboxamide. MS ESI calculated for C₂₅H₂₃F₄N₇O₂ [M+H]⁺, 530.18, found 530.25; ¹H NMR (400 MHz, CDCl₃) δ 7.97 (s, 1H), 7.18 (d, J=8.2 Hz, 1H), 7.07 (s, 1H), 6.52-6.37 (m, 2H), 6.34-6.13 (m, 1H), 5.80 (dd, J=8.6, 3.6 Hz, 1H), 5.45 (s, 1H), 5.32-5.31 (m, 1H), 4.70 (dd, J=28.9, 9.0 Hz, 1H), 4.20-4.17 (m, 1H), 4.06-4.03 (m, 1H), 3.41-3.37 (m, 1H), 3.05-3.02 (m, 3H), 2.75-2.70 (m, 1H), 2.63-2.60 (m, 1H), 1.29-1.17 (m, 2H), 1.17-1.05 (m, 2H).

### Example 164: 1-((3S,5R)-1-Acryloyl-5-methylpyrrolidin-3-yl)-3-((1-cyclopropyl-6-fluoro-1H-benzo[d]imidazol-5-yl)ethynyl)-5-(methylamino)-1H-pyrazole-4-carboxamide

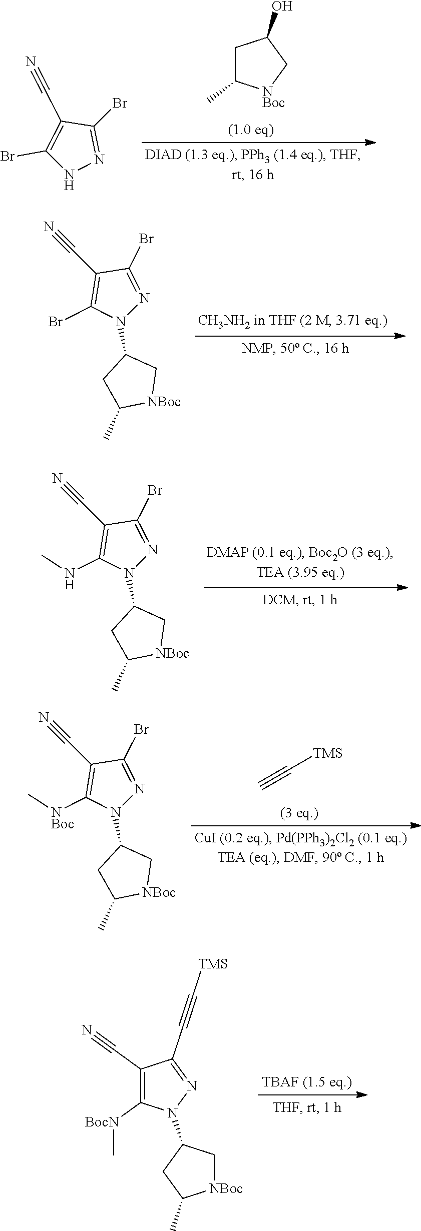

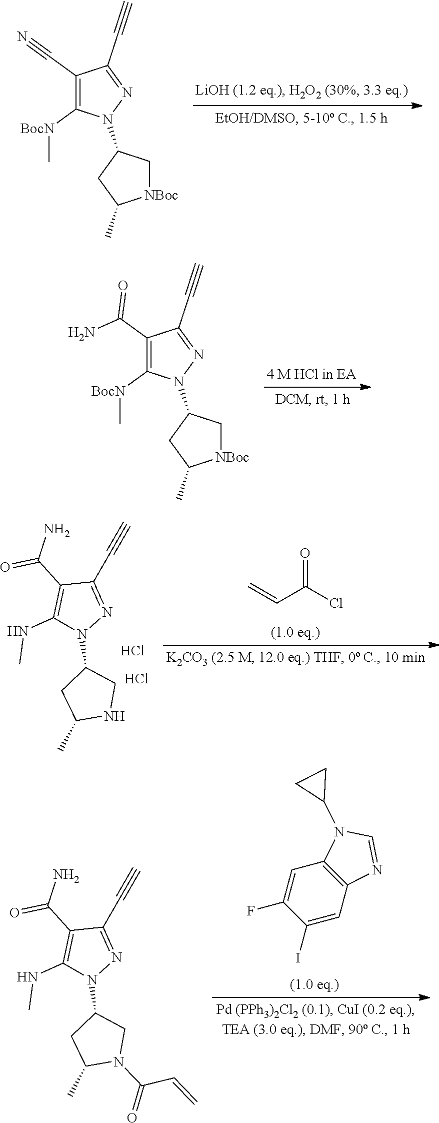

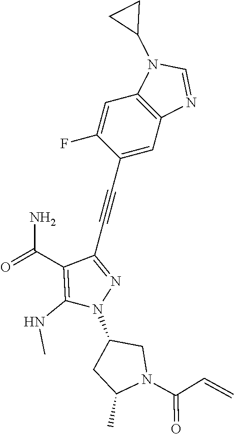 [SMILES: C=CC(=O)N1C[C@@H](n2nc(C#Cc3cc4ncn(C5CC5)c4cc3F)c(C(N)=O)c2NC)C[C@H]1C]

### Step 1: Tert-butyl (2R,4S)-4-(3,5-dibromo-4-cyanopyrazol-1-yl)-2-methylpyrrolidine-1-carboxylate

To a stirred solution of 3,5-dibromo-1H-pyrazole-4-carbonitrile (21.19 g, 84.47 mmol), tert-butyl(2R,4R)-4-hydroxy-2-methylpyrrolidine-1-carboxylate (17 g, 84.47 mmol) and PPh3 (33.23 g, 0.12 mol) in THF (170.00 mL) was added DIAD (22.20 g, 0.11 mol) dropwise at 0° C. The reaction mixture was degassed with nitrogen for three times and stirred for 16 h at room temperature. The resulting mixture was concentrated under reduced pressure. The residue was purified by silica gel column chromatography, eluted with PE/EA (4/1). The fractions contained desired product were combined and concentrated to afford tert-butyl (2R,4S)-4-(3,5-dibromo-4-cyanopyrazol-1-yl)-2-methylpyrrolidine-1-carboxylate (42.70 g, 87%) as an off-white solid. MS ESI calculated for C14H18Br2N4O2 [M+H−56]+, 376.98, 378.98, 380.98, found 376.80, 378.80, 380.80; 1H NMR (400 MHz, CDCl3) δ 5.06-4.80 (m, 2H), 4.07 (d, J=51.2 Hz, 1H), 3.88-3.47 (m, 1H), 2.64-2.53 (m, 1H), 2.28 (s, 1H), 1.44-1.33 (m, 3H), 1.29 (s, 9H).

### Step 2: Tert-butyl (2S,4S)-4-[3-bromo-4-cyano-5-(methylamino)pyrazol-1-yl]-2-methylpyrrolidine-1-carboxylate

To a stirred solution of tert-butyl (2R,4S)-4-(3,5-dibromo-4-cyanopyrazol-1-yl)-2-methylpyrrolidine-1-carboxylate (44.65 g, 0.10 mol) in NMP (10.00 mL) was added CH3NH2 (0.19 L, 0.39 mol, 2 M in THF). The reaction mixture was stirred for 16 h at 50° C. under nitrogen atmosphere. The resulting mixture was concentrated under reduced pressure. The residue was purified by reverse phase chromatography with the following conditions: column: Spherical C18, 20-40 μm, 330 g; Mobile Phase A: water (10 mmol/L NH₄HCO₃), Mobile Phase B: ACN; Flow rate: 80 mL/min; Gradient (B %): 30%˜60% within 30 min; Detector: UV 254/210 nm. The fractions contained desired product were combined and concentrated to afford tert-butyl (2S,4S)-4-[3-bromo-4-cyano-5-(methylamino)pyrazol-1-yl]-2-methylpyrrolidine-1-carboxylate (24.30 g, 58%) as an off-white solid. MS ESI calculated for C₁₅H₂₂BrN₅O₂ [M+H−56]+, 328.10, 330.09, found 328.00, 330.00; 1H NMR (400 MHz, CDCl3) δ 4.89 (s, 1H), 4.37-4.24 (m, 1H), 3.98-3.88 (m, 1H), 3.91-3.80 (m, 1H), 3.40-3.35 (m, 1H), 3.18 (d, J=5.1 Hz, 3H), 2.44-2.39 (m, 1H), 2.37-2.24 (m, 1H), 1.46 (s, 9H), 1.35 (d, J=6.2 Hz, 3H).

### Step 3: Tert-butyl (2R,4S)-4-{3-bromo-5-[(tert-butoxycarbonyl)(methyl)amino]-4-cyanopyrazol-1-yl}-2-methylpyrrolidine-1-carboxylate

To a stirred solution of tert-butyl (2R,4S)-4-[3-bromo-4-cyano-5-(methylamino)pyrazol-1-yl]-2-methylpyrrolidine-1-carboxylate (24.30 g, 63.24 mmol) and DMAP (0.77 g, 6.32 mmol) in DCM (243.00 mL) were added TEA (35.16 mL, 0.25 mol) and Boc₂O (41.40 g, 0.19 mol) dropwise at 0° C. under nitrogen atmosphere. The reaction mixture was stirred for 1 h at room temperature under nitrogen atmosphere. The resulting mixture was concentrated under reduced pressure. The residue was purified by silica gel column chromatography, eluted with PE/EA (4/1). The fractions contained desired product were combined and concentrated to afford tert-butyl (2R,4S)-4-{3-bromo-5-[(tert-butoxycarbonyl)(methyl)amino]-4-cyanopyrazol-1-yl}-2-methylpyrrolidine-1-carboxylate (26.80 g, 78%) as an off-white solid. MS ESI calculated for C20H30BrN5O4 [M+H−56]+, 428.15, 430.15, found 428.10, 430.10; 1H NMR (400 MHz, CDCl3) δ 4.53-4.45 (m, 1H), 3.96 (s, 2H), 3.56-3.48 (m, 1H), 3.23 (s, 3H), 2.51-2.44 (m, 1H), 2.23 (s, 1H), 1.47 (s, 18H), 1.42-1.36 (m, 3H).

### Step 4: Tert-butyl (2R,4S)-4-{5-[(tert-butoxycarbonyl)(methyl)amino]-4-cyano-3-[2-(trimethylsilyl)ethynyl]pyrazol-1-yl}-2-methylpyrrolidine-1-carboxylate

To a stirred mixture of tert-butyl (2R,4S)-4-{3-bromo-5-[(tert-butoxycarbonyl)(methyl)amino]-4-cyanopyrazol-1-yl}-2-methylpyrrolidine-1-carboxylate (26.80 g, 55.33 mmol), Pd(PPh3)2Cl2 (3.88 g, 5.53 mmol) and CuI (2.11 g, 11.07 mmol) in DMF (268.00 mL) were added TEA (23.07 mL, 0.16 mol) and trimethylsilylacetylene (23.46 mL, 0.16 mol) at room temperature under nitrogen atmosphere. The reaction mixture was degassed with nitrogen for three times and stirred for 1 h at 90° C. The resulting mixture was concentrated under reduced pressure. The residue was purified by silica gel column chromatography, eluted with 23% EA in PE. The fractions contained desired product were combined and concentrated tert-butyl (2R,4S)-4-{5-[(tert-butoxycarbonyl)(methyl)amino]-4-cyano-3-[2-(trimethylsilyl)ethynyl]pyrazol-1-yl}-2-methylpyrrolidine-1-carboxylate (22.30 g, 72%) as a brown solid. MS ESI calculated for C25H39N5O4Si [M+H]+, 502.28, found 502.35; 1H NMR (400 MHz, CDCl3) δ 4.53-4.46 (m, 1H), 3.98-3.92 (m, 2H), 3.68-3.41 (m, 1H), 3.24 (d, J=4.4 Hz, 3H), 2.49 (s, 1H), 2.30-2.25 (m, 1H), 1.48-1.45 (m, 18H), 1.41 (d, J=5.9 Hz, 3H), 0.30 (s, 9H).

### Step 5: Tert-butyl (2R,4S)-4-{5-[(tert-butoxycarbonyl)(methyl)amino]-4-cyano-3-ethynylpyrazol-1-yl}-2-methylpyrrolidine-1-carboxylate

To a stirred solution of tert-butyl (2R,4S)-4-{5-[(tert-butoxycarbonyl)(methyl)amino]-4-cyano-3-[2-(trimethylsilyl)ethynyl]pyrazol-1-yl}-2-methylpyrrolidine-1-carboxylate (22.30 g, 44.45 mmol) in THF (223.00 mL) was added 1 M TBAF in THF (66.67 mL, 66.67 mmol) at 0° C. under nitrogen atmosphere. The reaction mixture was stirred for 1 h at room temperature. The resulting mixture was concentrated under reduced pressure. The residue was purified by silica gel column chromatography, eluted with 25% EA in PE. The fractions contained desired product were combined and concentrated to afford tert-butyl (2R,4S)-4-{5-[(tert-butoxycarbonyl)(methyl)amino]-4-cyano-3-ethynylpyrazol-1-yl}-2-methylpyrrolidine-1-carboxylate (14.30 g, 67%) as an off-white solid. MS ESI calculated for C22H31N5O4 [M+H−112]+, 318.24, found 318.05; 1H NMR (400 MHz, CDCl3) δ 4.56-4.51 (m, 1H), 3.99-3.95 (m, 2H), 3.74-3.44 (m, 1H), 3.39-3.32 (m, 1H), 3.25 (s, 3H), 2.63-2.39 (m, 1H), 2.38-2.14 (m, 1H), 1.54-1.49 (m, 18H), 1.40 (d, J=7.0 Hz, 3H).

### Step 6: Tert-butyl (2R,4S)-4-{5-[(tert-butoxycarbonyl)(methyl)amino]-4-carbamoyl-3-ethynylpyrazol-1-yl}-2-methylpyrrolidine-1-carboxylate

To a stirred solution of tert-butyl (2R,4S)-4-{5-[(tert-butoxycarbonyl)(methyl)amino]-4-cyano-3-ethynylpyrazol-1-yl}-2-methylpyrrolidine-1-carboxylate (14.30 g, 33.29 mmol) in EtOH (235.00 mL) and DMSO (47.00 mL) were added 0.5 M LiOH (79.90 mL, 39.95 mmol) and H₂O₂ (30%, 9.05 mL, 0.11 mol) at 0° C. The reaction mixture was stirred for 1.5 h at 5-10° C. The resulting mixture was quenched with sat. Na2SO3 (aq.) (2.5 M) at 0° C. The resulting mixture was diluted with water (500 mL) and extracted with EA (3×500 mL). The combined organic layers was washed with brine (5×300 mL), dried over anhydrous Na2SO4, filtered and concentrated under reduced pressure. The residue was purified by silica gel column chromatography, eluted with 63% EA in PE. The fractions contained desired product were combined and concentrated to afford tert-butyl (2R,4S)-4-{5-[(tert-butoxycarbonyl)(methyl)amino]-4-carbamoyl-3-ethynylpyrazol-1-yl}-2-methylpyrrolidine-1-carboxylate (16.50 g, 88%) as a light yellow solid. MS ESI calculated for C22H33N5O5 [M+H−156]+, 292.25, found 292.25; 1H NMR (400 MHz, CDCl3) δ 6.84 (s, 1H), 5.68 (s, 1H), 4.77-4.68 (m, 1H), 4.07-3.37 (m, 4H), 3.08-3.02 (m, 6H), 2.48-2.42 (m, 1H), 1.87 (s, 1H), 1.58-1.32 (m, 18H).

### Step 7: 3-Ethynyl-5-(methylamino)-1-[(3S,5R)-5-methylpyrrolidin-3-yl]pyrazole-4-carboxamide dihydrochloride

To a stirred mixture of tert-butyl (2R,4S)-4-{5-[(tert-butoxycarbonyl)(methyl)amino]-4-carbamoyl-3-ethynylpyrazol-1-yl}-2-methylpyrrolidine-1-carboxylate (16.50 g, 36.87 mmol) in DCM (165.00 mL) was added 4 M HCl in EA (165.00 mL, 0.66 mol). The reaction mixture was stirred for 1 h at room temperature. The precipitated solids were collected by filtration and washed with CH2Cl2 (3×50 mL). The filter cake was dried to afford 3-ethynyl-5-(methylamino)-1-[(3S,5R)-5-methylpyrrolidin-3-yl]pyrazole-4-carboxamide dihydrochloride (9.50 g, 68%) as a light yellow solid which was used in the next step directly without further purification. MS ESI calculated for C12H19Cl2N5O [M+H−2HCl]+, 248.14, found 248.05.

### Step 8: 3-Ethynyl-1-[(3S,5R)-5-methyl-1-(prop-2-enoyl)pyrrolidin-3-yl]-5-(methylamino)pyrazole-4-carboxamide

To a stirred mixture of 3-ethynyl-5-(methylamino)-1-[(3S,5R)-5-methylpyrrolidin-3-yl]pyrazole-4-carboxamide dihydrochloride (2.50 g, 7.81 mmol) and acryloyl chloride (0.71 g, 7.81 mmol) in THF (25.00 mL) was added 2.5 M K2CO3 (37.47 mL, 93.68 mmol) dropwise at 0° C. under nitrogen atmosphere. The resulting mixture was stirred for 10 min at 0° C. under nitrogen atmosphere. The resulting mixture was washed with 3×50 mL of water. The resulting mixture was extracted with EA (3×100 mL). The combined organic layers was washed with brine (2×50 mL), dried over anhydrous Na2SO4. After filtration, the filtrate was concentrated under reduced pressure. The residue was purified by silica gel column chromatography, eluted with CH2Cl2/MeOH (20/1). The fractions contained desired product were combined and concentrated to afford 3-ethynyl-1-[(3S,5R)-5-methyl-1-(prop-2-enoyl)pyrrolidin-3-yl]-5-(methylamino)pyrazole-4-carboxamide (2.40 g, 96%) as a white solid. MS ESI calculated for C15H19N5O2 [M+H]+, 302.15, found 302.10; 1H NMR (400 MHz, CDCl3) δ 6.81 (s, 1H), 6.54-6.42 (m, 1H), 6.41-6.30 (m, 1H), 5.84-5.64 (m, 1H), 5.42 (s, 1H), 4.86-4.81 (m, 1H), 4.50-3.76 (m, 3H), 3.48 (d, J=4.2 Hz, 1H), 2.98 (s, 3H), 2.74-2.54 (m, 1H), 2.52-2.28 (m, 1H), 1.45 (d, J=6.2 Hz, 3H).

### Step 9: 1-((3S,5R)-1-Acryloyl-5-methylpyrrolidin-3-yl)-3-((1-cyclopropyl-6-fluoro-1H-benzo[d]imidazol-5-yl)ethynyl)-5-(methylamino)-1H-pyrazole-4-carboxamide

To a stirred mixture of 3-ethynyl-1-[(3S,5R)-5-methyl-1-(prop-2-enoyl)pyrrolidin-3-yl]-5-(methylamino)pyrazole-4-carboxamide (0.25 g, 0.83 mmol), Pd(PPh3)2Cl2 (58.23 mg, 0.08 mmol), 1-cyclopropyl-6-fluoro-5-iodo-1,3-benzodiazole (0.25 g, 0.83 mmol) and CuI (31.60 mg, 0.17 mmol) in DMF (5.00 mL) was added TEA (0.35 mL, 2.49 mmol). The reaction mixture was degassed with argon for three timed and stirred for 1 h at 90° C. The resulting mixture was washed with 3×20 mL of water. The resulting mixture was extracted with EA (3×50 mL). The combined organic layers was washed with brine (3×20 mL), dried over anhydrous Na2SO4. After filtration, the filtrate was concentrated under reduced pressure. The residue was purified by silica gel column chromatography, eluted with CH2Cl2/MeOH (10/1) to afford the crude product which was further purified by reverse flash chromatography with the following conditions: column: C18 silica gel; mobile phase: ACN in water (10 mmol/L NH4HCO3), 40% to 60% gradient in 20 min; detector: UV 254 nm. The fractions contained desired product were combined and concentrated to afford 1-((3S,5R)-1-acryloyl-5-methylpyrrolidin-3-yl)-3-((1-cyclopropyl-6-fluoro-1H-benzo[d]imidazol-5-yl)ethynyl)-5-(methylamino)-1H-pyrazole-4-carboxamide (0.14 g, 36%) as a white solid. MS ESI calculated for C25H26FN7O2 [M+H]+, 476.21, found 476.20; 1H NMR (400 MHz, CDCl3) δ 8.08-7.86 (m, 2H), 7.42-7.24 (m, 1H), 7.15 (s, 1H), 6.68 (s, 1H), 6.59-6.27 (m, 2H), 5.83-5.64 (m, 1H), 5.50 (s, 1H), 4.83 (q, J=8.5 Hz, 1H), 4.57-3.80 (m, 3H), 3.39-3.32 (m, 1H), 2.99 (d, J=5.6 Hz, 3H), 2.77-2.26 (m, 2H), 1.48 (d, J=6.2 Hz, 3H), 1.29-1.16 (m, 2H), 1.14-0.98 (m, 2H).

### Example 165: 3-[2-(1-Cyclopropyl-4,6-difluoro-1,3-benzodiazol-5-yl)ethynyl]-1-[(3S,5R)-5-methyl-1-(prop-2-enoyl)pyrrolidin-3-yl]-5-(methylamino)pyrazole-4-carboxamide

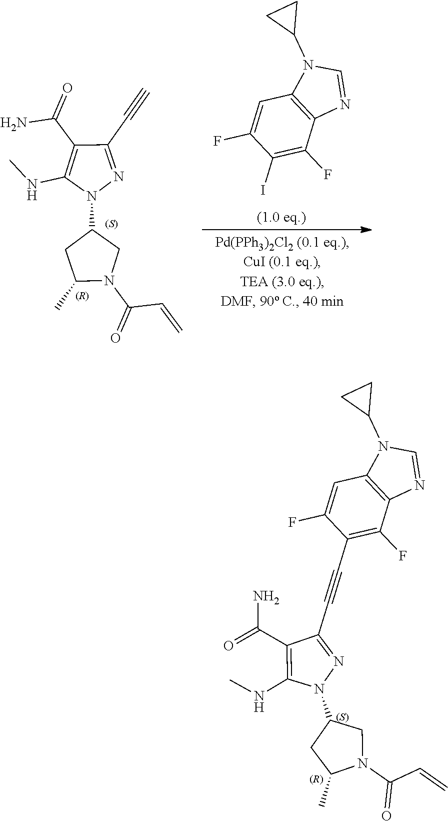

To a stirred solution of 3-ethynyl-1-[(3S,5R)-5-methyl-1-(prop-2-enoyl)pyrrolidin-3-yl]-5-(methylamino)pyrazole-4-carboxamide (0.30 g, 0.99 mmol), 1-cyclopropyl-4,6-difluoro-1,3-benzodiazole (0.19 g, 0.99 mmol), Pd(PPh3)2Cl2 (69.88 mg, 0.10 mmol) and CuI (37.92 mg, 0.20 mmol) in DMF (5.00 mL) was added TEA (0.42 mL, 2.98 mmol). The reaction mixture was degassed with argon for three times and stirred for 40 min at 90° C. The resulting mixture was cooled and concentrated under vacuum. The residue was purified by silica gel column chromatography, eluted with DCM/MeOH (10/1) to afford the crude product which was further purified by reverse phase flash with the following conditions: column, C18 silica gel; Mobile Phase A: water (10 mmol/L NH4HCO3), Mobile Phase B: ACN; Gradient: 20 B to 50 B in 30 min; 210/254 nm. The fractions contained desired product were combined and concentrated under reduced pressure to afford 3-[2-(1-cyclopropyl-4,6-difluoro-1,3-benzodiazol-5-yl)ethynyl]-1-[(3S,5R)-5-methyl-1-(prop-2-enoyl)pyrrolidin-3-yl]-5-(methylamino)pyrazole-4-carboxamide (0.10 g, 21%) as a white solid. MS ESI calculated for C25H25F2N7O2 [M+H]+, 494.20, found 494.10; 1H NMR (400 MHz, CDCl3) δ 7.97 (s, 1H), 7.26-6.99 (m, 2H), 6.68 (s, 1H), 6.58-6.29 (m, 2H), 5.82-5.61 (m, 1H), 5.43 (s, 1H), 4.92-4.77 (m, 1H), 4.56-4.09 (m, 2H), 4.09-3.80 (m, 1H), 3.48-3.36 (m, 1H), 3.00 (d, J=5.5 Hz, 3H), 2.83-2.29 (m, 2H), 1.49 (d, J=6.2 Hz, 3H), 1.32-1.25 (m, 2H), 1.19-1.05 (m, 2H).

### Example 166: 1-((3S,5R)-1-Acryloyl-5-methylpyrrolidin-3-yl)-3-((6-chloro-1-cyclopropyl-1H-benzo[d]imidazol-5-yl)ethynyl)-5-(methylamino)-1H-pyrazole-4-carboxamide

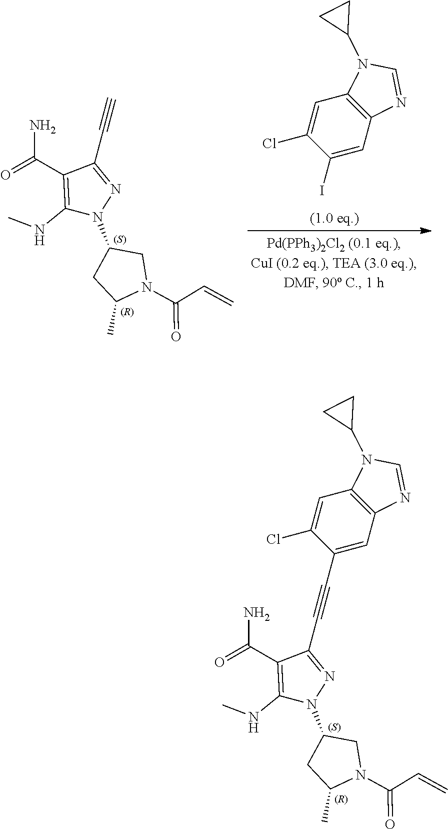

1-((3S,5R)-1-acryloyl-5-methylpyrrolidin-3-yl)-3-((6-chloro-1-cyclopropyl-1H-benzo[d]imidazol-5-yl)ethynyl)-5-(methylamino)-1H-pyrazole-4-carboxamide. MS ESI calculated for C₂₅H₂₆ClN₇O₂[M+H]⁺, 492.18, found 492.25; ¹H NMR (400 MHz, CDCl₃) δ 8.10 (s, 1H), 8.02 (s, 1H), 7.68 (s, 1H), 7.22 (s, 1H), 6.67 (s, 1H), 6.54-6.36 (m, 2H), 5.81-5.67 (m, 1H), 5.45 (s, 1H), 4.84 (p, J=8.6, 7.9 Hz, 1H), 4.57-4.10 (m, 2H), 4.09-3.79 (m, 1H), 3.46-3.40 (m, 1H), 3.00 (s, 3H), 2.76-2.30 (m, 2H), 1.49 (d, J=6.2 Hz, 3H), 1.31-1.19 (m, 2H), 1.13-1.04 (m, 2H).

### Example 167: 5-Amino-3-[2-(1-cyclopropyl-6-fluoro-1,3-benzodiazol-5-yl)ethynyl]-1-[(3S,5R)-5-(methoxymethyl)-1-(prop-2-enoyl)pyrrolidin-3-yl]pyrazole-4-carboxamide

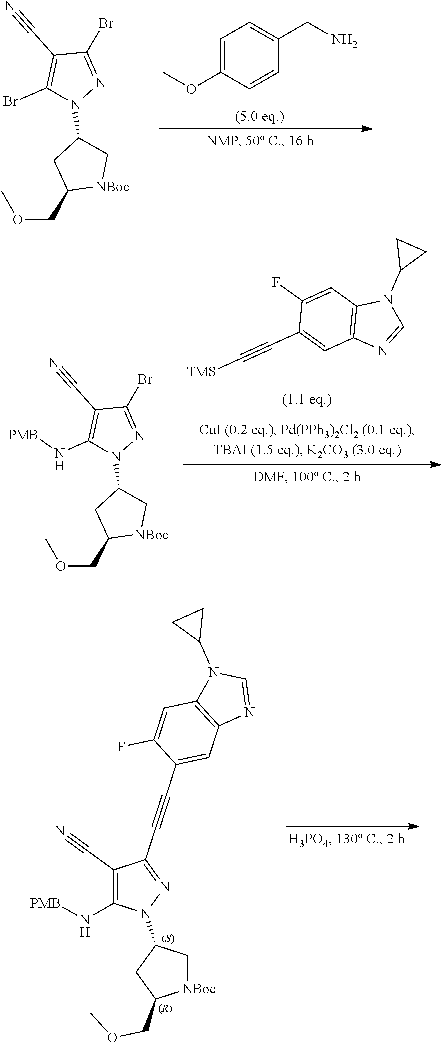

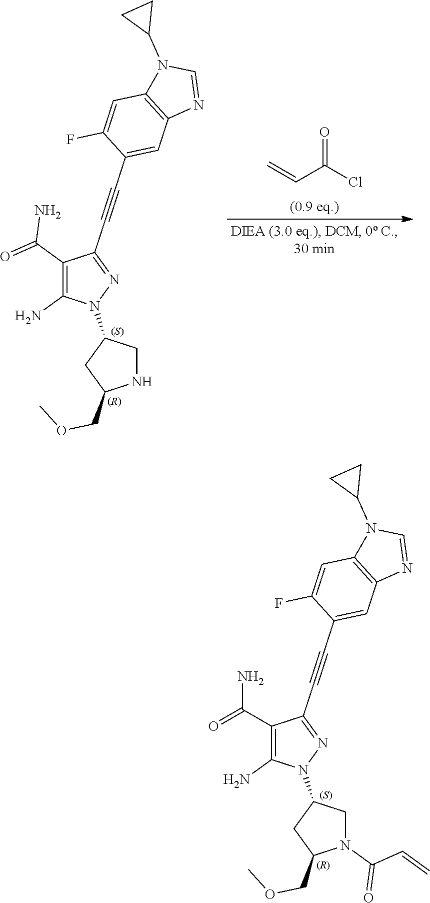

### Step 1: Tert-butyl (2R,4S)-4-(3-bromo-4-cyano-5-((4-methoxybenzyl)amino)-1H-pyrazol-1-yl)-2-(methoxymethyl)pyrrolidine-1-carboxylate

To a stirred solution of tert-butyl (2R,4S)-4-(3,5-dibromo-4-cyanopyrazol-1-yl)-2-(methoxymethyl)pyrrolidine-1-carboxylate (1.00 g, 2.15 mmol) and (4-methoxyphenyl)methanamine (1.48 g, 10.77 mmol) in NMP (10.00 mL) at room temperature under nitrogen atmosphere. The resulting mixture was stirred for 16 h at 50° C. under nitrogen atmosphere. The residue was purified by reverse flash chromatography with the following conditions: column: C18 silica gel; mobile phase: MeOH in water (10 mmol/L NH4HCO3), 10% to 50% gradient in 10 min; detector: UV 210 nm. The fractions contained desired product were combined and concentrated to afford tert-butyl (2R,4S)-4-(3-bromo-4-cyano-5-((4-methoxybenzyl)amino)-1H-pyrazol-1-yl)-2-(methoxymethyl)pyrrolidine-1-carboxylate (0.70 g, 62%) as an off-white solid. MS ESI calculated for C23H30BrN5O4 [M+H]+, 520.15, 522.15, found 520.15, 522.15; 1H NMR (400 MHz, CDCl3) δ 7.28-7.10 (d, J=1.8 Hz, 1H), 6.90-6.86 (m, 1H), 3.80-3.78 (s, 1H), 3.39-3.34 (m, 6H), 2.82-2.80 (d, J=0.8 Hz, 9H), 2.34-2.31 (t, J=8.1 Hz, 6H), 2.05-1.96 (m, 6H).

### Step 2: Tert-butyl (2R,4S)-4-{4-cyano-3-[2-(1-cyclopropyl-6-fluoro-1,3-benzodiazol-5-yl)ethynyl]-5-{[(4-methoxyphenyl)methyl]amino}pyrazol-1-yl}-2-(methoxymethyl)pyrrolidine-1-carboxylates

To a stirred mixture of tert-butyl (2R,4S)-4-(3-bromo-4-cyano-5-((4-methoxybenzyl)amino)-1H-pyrazol-1-yl)-2-(methoxymethyl)pyrrolidine-1-carboxylate (1.00 g, 1.92 mmol), Pd(PPh3)2Cl2 (0.13 g, 0.19 mmol), K2CO3 (0.80 g, 5.76 mmol), CuI (73.19 mg, 0.38 mmol) and TBAI (1.06 g, 2.88 mmol) in DMF (10.00 mL) was added 1-cyclopropyl-5-ethynyl-6-fluoro-1,3-benzodiazole (0.46 g, 2.30 mmol). The reaction mixture was degassed with argon for three times and stirred for 2 h at 100° C. The resulting mixture was cooled down and concentrated under vacuum. The residue was purified by silica gel column chromatography, eluted with DCM/MeOH (10/1). The fractions contained desired product were combined and concentrated under reduced pressure to afford tert-butyl (2R,4S)-4-{4-cyano-3-[2-(1-cyclopropyl-6-fluoro-1,3-benzodiazol-5-yl)ethynyl]-5-{[(4-methoxyphenyl)methyl]amino)pyrazol-1-yl}-2-(methoxymethyl)pyrrolidine-1-carboxylates (0.60 g, 48%) as a yellow solid. MS ESI calculated for C35H38FN7O4 [M+H]+, 640.30, found 640.55.

### Step 3: 5-Amino-3-[2-(1-cyclopropyl-6-fluoro-1,3-benzodiazol-5-yl)ethynyl]-1-[(3S,5R)-5-(methoxymethyl)pyrrolidin-3-yl]pyrazole-4-carboxamide

A solution of tert-butyl (2R,4S)-4-{4-cyano-3-[2-(1-cyclopropyl-6-fluoro-1,3-benzodiazol-5-yl)ethynyl]-5-{[(4-methoxyphenyl)methyl]amino}pyrazol-1-yl}-2-(methoxymethyl)pyrrolidine-1-carboxylates (0.60 g, 0.94 mmol) in H3PO4 (2.00 mL) was stirred for 2 h at 130° C. The reaction mixture was diluted with ice water (20 mL). The resulting mixture was extracted with EA (3×20 mL). The combined organic layers was washed with water (2×10 mL), dried over anhydrous Na2SO4. After filtration, the filtrate was concentrated under reduced pressure. The residue was purified by silica gel column chromatography, eluted with DCM/MeOH (15/1). The fractions contained desired product were combined and concentrated under reduced pressure to afford 5-amino-3-[2-(1-cyclopropyl-6-fluoro-1,3-benzodiazol-5-yl)ethynyl]-1-[(3S,5R)-5-(methoxymethyl)pyrrolidin-3-yl]pyrazole-4-carboxamide (0.26 g, 63%) as a yellow solid. MS ESI calculated for C22H24FN7O2 [M+H]+, 438.20, found 438.25.

### Step 4: 5-Amino-3-[2-(1-cyclopropyl-6-fluoro-1,3-benzodiazol-5-yl)ethynyl]-1-[(3S,5R)-5-(methoxymethyl)-1-(prop-2-enoyl)pyrrolidin-3-yl]pyrazole-4-carboxamide

To a stirred solution of 5-amino-3-[2-(1-cyclopropyl-6-fluoro-1,3-benzodiazol-5-yl)ethynyl]-1-[(3S,5R)-5-(methoxymethyl)pyrrolidin-3-yl]pyrazole-4-carboxamide (0.26 g, 0.59 mmol) in DCM (4.00 mL) and DIEA (0.31 mL, 1.78 mmol) was added acryloyl chloride (48.41 mg, 0.53 mmol) dropwise at 0° C. The reaction mixture was stirred for 30 min at 0° C. The resulting mixture was cooled down and concentrated under vacuum. The residue was purified by reverse phase flash with the following conditions: column: C₁₈ silica gel; mobile phase: ACN in water (10 mmol/L NH4HCO3), 10% to 50% gradient in 30 min; detector: UV 254 nm. The fractions contained desired product were combined and concentrated under reduced pressure to afford 5-amino-3-[2-(1-cyclopropyl-6-fluoro-1,3-benzodiazol-5-yl)ethynyl]-1-[(3S,5R)-5-(methoxymethyl)-1-(prop-2-enoyl)pyrrolidin-3-yl]pyrazole-4-carboxamide (0.13 g, 44%) as a white solid. MS ESI calculated for C25H26FN7O3 [M+H]+, 492.21, found 492.15; 1H NMR (400 MHz, CDCl3) δ 7.98 (brs, 2H), 7.33 (d, J=9.1 Hz, 1H), 7.09 (s, 1H), 6.61-6.29 (i, 2H), 5.83-5.37 (m, 4H), 5.23-4.83 (m, 1H), 4.65-4.29 (m, 1H), 4.13-3.81 (m, 3H), 3.61-3.29 (m, 5H), 3.09-2.61 (m, 1H), 2.38-2.36 (m, 1H), 1.32-1.00 (i, 4H).

### Example 168: 5-Amino-3-[2-(1-cyclopropyl-6-fluoro-1,3-benzodiazol-5-yl)ethynyl]-1-[(3 S)-1-(prop-2-enoyl)pyrrolidin-3-yl]pyrazole-4-carboxamide

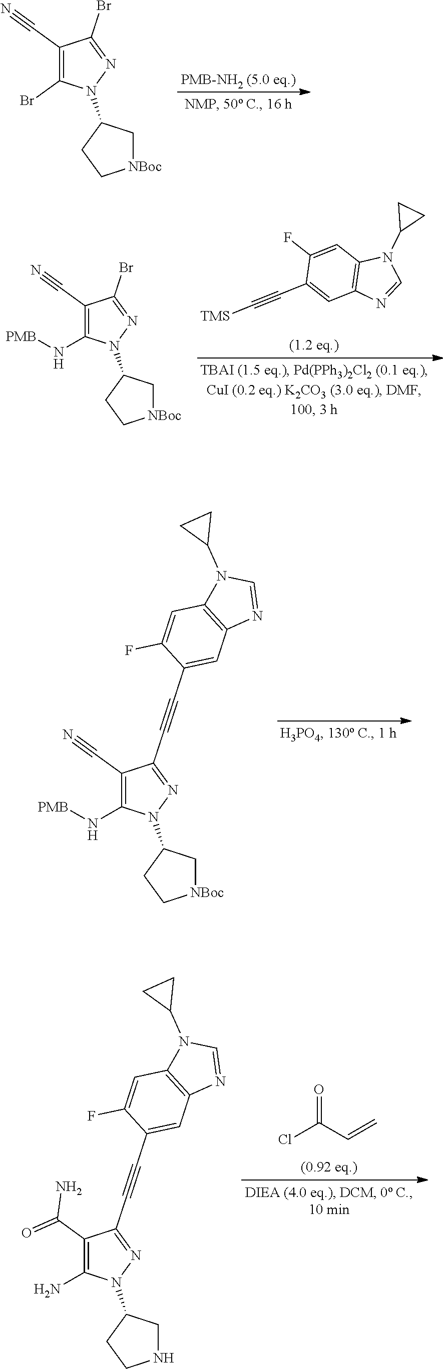

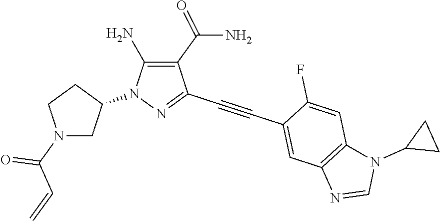 [SMILES: C=CC(=O)N1CC[C@H](n2nc(C#Cc3cc4ncn(C5CC5)c4cc3F)c(C(N)=O)c2N)C1]

### Step 1: Tert-butyl-(3S)-3-(3-bromo-4-cyano-5-1[(4-methoxyphenyl)methyl]amino) pyrazol-1-yl)pyrrolidine-1-carboxylate

To a stirred solution of tert-butyl (3S)-3-(3,5-dibromo-4-cyanopyrazol-1-yl)pyrrolidine-1-carboxylate (5.00 g, 11.90 mmol) in NMP (50.00 mL) was added (4-methoxyphenyl)methanamine (8.16 g, 59.51 mmol) at room temperature. The reaction mixture was stirred for 16 h at 50° C. The resulting mixture was diluted with EA (50.00 mL). The residue was washed with water (3×30 mL). The combined organic layers was washed with brine (2×30 mL), dried over anhydrous Na2SO4. After filtration, the filtrate was concentrated under reduced pressure. The residue was purified by silica gel column chromatography, eluted with 35% EA in PE. The fractions contained desired product were combined and concentrated to afford tert-butyl (3S)-3-(3-bromo-4-cyano-5-{[(4-methoxyphenyl)methyl]amino}pyrazol-1-yl)pyrrolidine-1-carboxylate (4.80 g, 84%) as a light yellow oil. MS ESI calculated for C13H16Br2N₄O₂ [M+H−100]+, 376.12, found 376.15.

### Step 2: Tert-butyl (3S)-3-{4-cyano-3-[2-(1-cyclopropyl-6-fluoro-1,3-benzodiazol-5-yl)ethynyl]-5-{[(4-methoxyphenyl)methyl]amino}pyrazol-1-yl}pyrrolidine-1-carboxylate

To a stirred solution of tert-butyl (3S)-3-(3-bromo-4-cyano-5-{[(4-methoxyphenyl)methyl]amino}pyrazol-1-yl)pyrrolidine-1-carboxylate (2.00 g, 4.19 mmol) and 1-cyclopropyl-6-fluoro-5-[2-(trimethylsilyl)ethynyl]-1,3-benzodiazole (1.37 g, 5.04 mmol) in DMF (20.00 mL) were added 2.5 M K2CO3 (1.74 g, 12.59 mmol), TBAI (2.33 g, 6.29 mmol), CuI (0.16 g, 0.84 mmol) and Pd(PPh3)2Cl2 (0.29 g, 0.42 mmol). The reaction mixture was degassed with argon for three times and stirred for 3 h at 100° C. The resulting mixture was concentrated under reduced pressure. The residue was purified by silica gel column chromatography, eluted with 4% MeOH in DCM to afford the crude product which was further purified by reverse phase flash with the following conditions: column: Spherical C18, 20-40 μm, 40 g; Mobile Phase A: water (10 mmol/L NH₄HCO₃); Mobile Phase B: ACN; Flow rate: 60 mL/min; Gradient (B %): 0% to 46% within 35 min; Detector: UV 254/220 nm. The fractions were combined and concentrated under reduced pressure to afford tert-butyl (3S)-3-{4-cyano-3-[2-(1-cyclopropyl-6-fluoro-1,3-benzodiazol-5-yl)ethynyl]-5-{[(4-methoxyphenyl)methyl]amino}pyrazol-1-yl}pyrrolidine-1-carboxylate (1.36 g, 54%) as a light yellow solid. MS ESI calculated for C33H34FN7O3 [M+H]+ 596.27; found 596.35; 1H NMR (400 MHz, CDCl3) δ 7.96-7.93 (m, 2H), 7.38-7.30 (m, 3H), 6.90 (d, J=8.2 Hz, 2H), 4.71 (s, 1H), 3.82 (s, 3H), 3.78-3.61 (m, 3H), 3.49-3.32 (m, 2H), 3.26 (s, 2H), 2.49-2.19 (m, 1H), 1.46 (s, 9H), 1.22-1.19 (m, 2H), 1.09-1.05 (m, 2H), 1.03-0.98 (m, 2H).

### Step 3: (S)-5-Amino-3-((1-cyclopropyl-6-fluoro-1H-benzo[d]imidazol-5-yl)ethynyl)-1-(pyrrolidin-3-yl)-1H-pyrazole-4-carboxamide

A solution of tert-butyl (3S)-3-{4-cyano-3-[2-(1-cyclopropyl-6-fluoro-1,3-benzodiazol-5-yl)ethynyl]-5-{[(4-methoxyphenyl)methyl]amino}pyrazol-1-yl}pyrrolidine-1-carboxylate (0.55 g, 0.92 mmol) in H3PO4 (5.50 mL) was stirred for 1 h at 130° C. The residue was basified to pH 8 with NaOH (aq.). The residue was purified by reverse phase flash with the following conditions: column: Spherical C18, 20-40 μm, 40 g; Mobile Phase A: water (10 mmol/L NH4HCO3), Mobile Phase B: ACN; Flow rate: 60 mL/min; Gradient (B %): 0% hold 5 min, 5% to 22% within 10 min, 22% hold 5 min, 22% to 45% within 20 min, 45% hold 3 min, 45% to 95% within 2 min, 95% hold 5 min; Detector: UV 254/210 nm. The fractions were combined and concentrated under reduced pressure to afford (S)-5-amino-3-((1-cyclopropyl-6-fluoro-1H-benzo[d]imidazol-5-yl)ethynyl)-1-(pyrrolidin-3-yl)-1H-pyrazole-4-carboxamide (0.27 g, 73%) as a light green solid. MS ESI calculated for C20H20FN7O [M+H]+, 394.17, found 394.10; 1H NMR (400 MHz, CDCl3) δ 8.11-8.01 (m, 1H), 8.00-7.89 (m, 2H), 7.31 (d, J=8.9 Hz, 1H), 6.83 (d, J=9.4 Hz, 1H), 5.30 (s, 1H), 4.89 (s, 1H), 3.39-3.36 (m, 1H), 2.98 (s, 1H), 2.90 (s, 1H), 2.14 (s, 1H), 1.69 (s, 2H), 1.46 (s, 1H), 1.32-1.13 (m, 2H), 1.11-0.99 (m, 4H).

### Step 4: 5-Amino-3-[2-(1-cyclopropyl-6-fluoro-1,3-benzodiazol-5-yl)ethynyl]-1-[(3S)-1-(prop-2-enoyl)pyrrolidin-3-yl]pyrazole-4-carboxamide

To a stirred solution of (S)-5-amino-3-((1-cyclopropyl-6-fluoro-1H-benzo[d]imidazol-5-yl)ethynyl)-1-(pyrrolidin-3-yl)-1H-pyrazole-4-carboxamide (0.27 g, 0.68 mmol) and DIEA (0.35 g, 2.72 mmol) in DCM (10.00 mL) was added acryloyl chloride (2.50 mL, 0.62 mmol) dropwise at 0° C. under nitrogen atmosphere. The reaction mixture was stirred for 10 min at 0° C. under nitrogen atmosphere. The reaction was quenched with water (10 mL) at 0° C. The resulting mixture was extracted with EA (3×30 mL). The combined organic layers was washed with brine (2×20 mL), dried over anhydrous Na2SO4. After filtration, the filtrate was concentrated under reduced pressure. The residue was purified by silica gel column chromatography, eluted with 6% MeOH in DCM to afford the crude product which was further purified by reverse phase chromatography with the following conditions: column: Spherical C18, 20-40 μm, 40 g; Mobile Phase A: water (10 mmol/L NH4HCO3), Mobile Phase B: ACN; Flow rate: 60 mL/min; Gradient (B %): 5% to 35% within 30 min; Detector: UV 254/210 nm. The collected fractions were combined and concentrated under reduced pressure to afford 5-amino-3-[2-(1-cyclopropyl-6-fluoro-1,3-benzodiazol-5-yl)ethynyl]-1-[(3S)-1-(prop-2-enoyl)pyrrolidin-3-yl]pyrazole-4-carboxamide (64.30 mg, 21%) as a white solid. MS ESI calculated for C23H22FN7O2 [M+H]+, 448.18, found 448.30; 1H NMR (400 MHz, CDCl3) δ 7.98 (d, J=6.6 Hz, 2H), 7.32 (d, J=9.0 Hz, 1H), 7.10 (s, 1H), 6.55-6.34 (m, 2H), 5.81-5.58 (m, 3H), 5.43 (s, 1H), 4.76-4.72 (m, 1H), 4.11-3.90 (m, 3H), 3.84-3.60 (m, 1H), 3.39-3.35 (m, 1H), 2.85-2.53 (m, 1H), 2.47-2.28 (m, 1H), 1.24-1.19 (m, 2H), 1.13-1.01 (m, 2H).

### Example 169: 1-((3S,5S)-1-Acryloyl-5-methylpyrrolidin-3-yl)-3-((1-cyclopropyl-6-fluoro-1H-benzo[d]imidazol-5-yl)ethynyl)-5-(methylamino)-1H-pyrazole-4-carboxamide

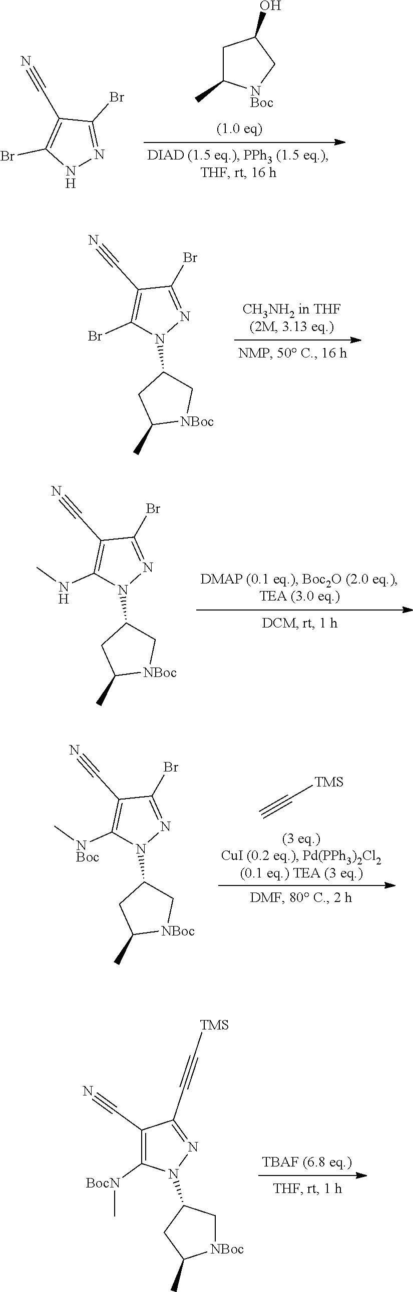

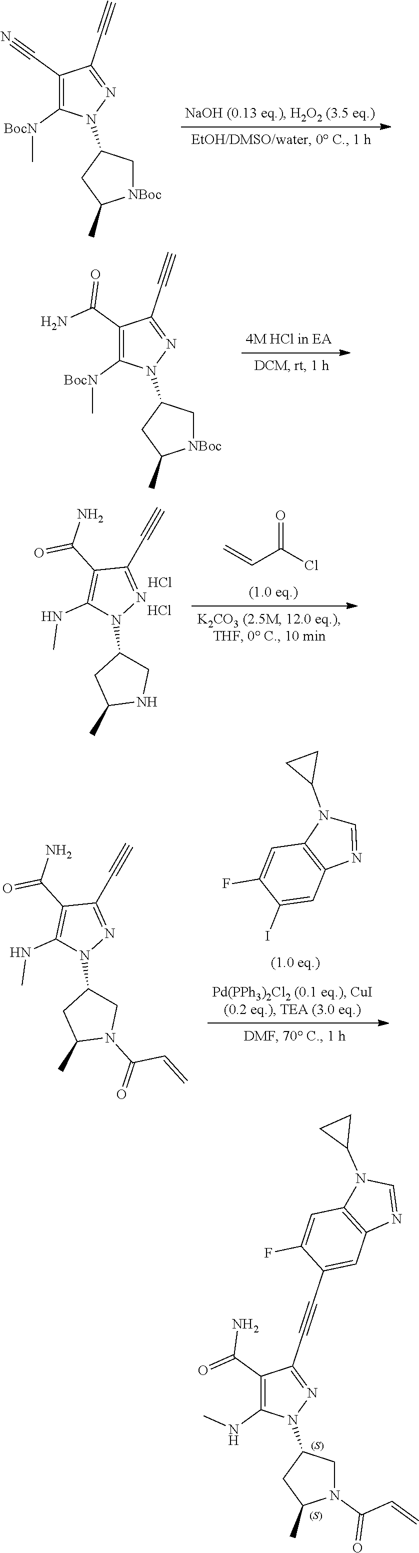

1-((3S,5S)-1-acryloyl-5-methylpyrrolidin-3-yl)-3-((1-cyclopropyl-6-fluoro-1H-benzo[d]imidazol-5-yl)ethynyl)-5-(methylamino)-1H-pyrazole-4-carboxamide. MS ESI calculated for C₂₅H₂₆FN₇O₂[M+H]+, 476.21, found 476.25; ¹H NMR (400 MHz, DMSO-d₆) δ 8.36 (s, 1H), 7.95 (d, J=6.2 Hz, 1H), 7.63 (d, J=9.4 Hz, 1H), 7.47 (s, 1H), 6.81 (s, 1H), 6.72-6.50 (m, 2H), 6.19-6.16 (m, 1H), 5.70-5.67 (m, 1H), 5.24-5.21 (m, 1H), 4.53-4.27 (m, 1H), 3.99-3.96 (m, 1H), 3.82 (d, J=6.8 Hz, 1H), 3.57-3.47 (m, 1H), 2.96 (dd, J=5.7, 1.2 Hz, 3H), 2.76-2.54 (m, 1H), 2.24-1.94 (m, 1H), 1.26 (dd, J=6.5, 2.3 Hz, 3H), 1.15-1.00 (m, 4H).

### Example 170: 3-[2-(1-Cyclopropyl-6-fluoro-1,3-benzodiazol-5-yl)ethynyl]-1-[(3S,5R)-5-(difluoromethyl)-1-(prop-2-enoyl)pyrrolidin-3-yl]-5-(methylamino)pyrazole-4-carboxamide

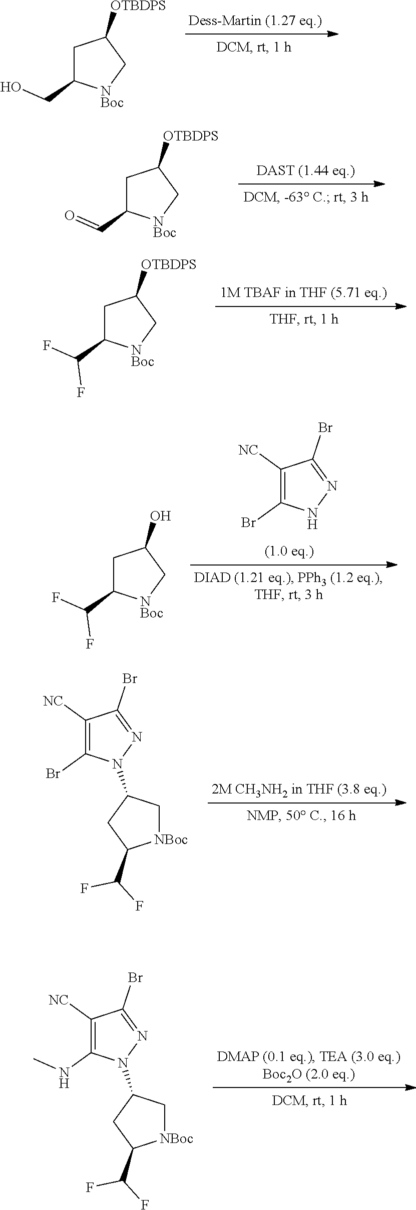

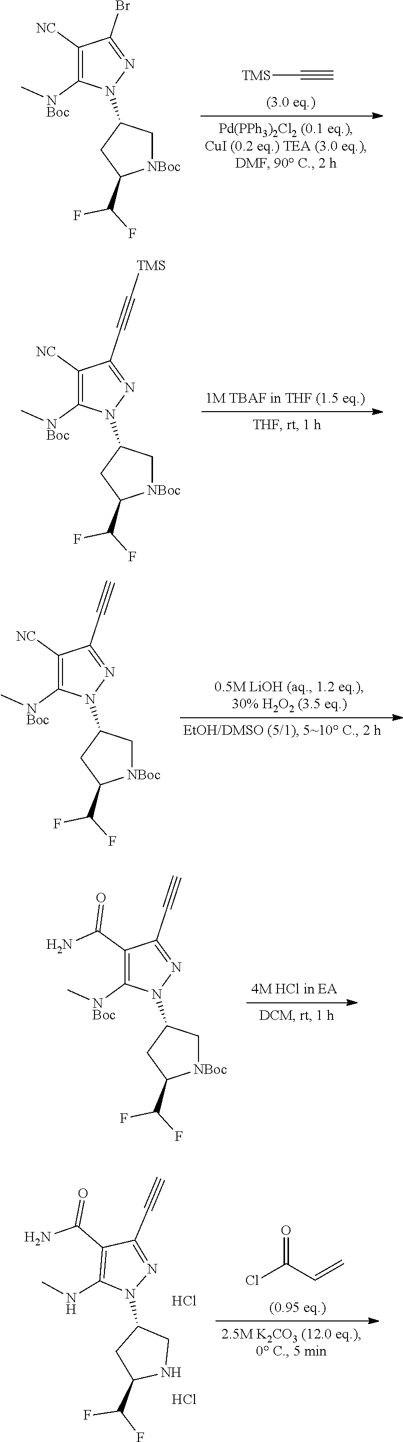

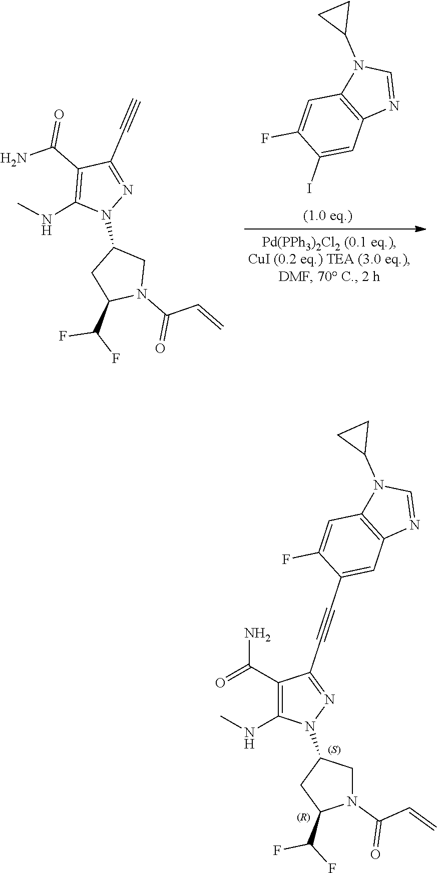

3-[2-(1-cyclopropyl-6-fluoro-1,3-benzodiazol-5-yl)ethynyl]-1-[(3S,5R)-5-(difluoromethyl)-1-(prop-2-enoyl)pyrrolidin-3-yl]-5-(methylamino)pyrazole-4-carboxamide. MS ESI calculated for C₂₅H₂₄F₃N₇O₂[M+H]+, 512.19, found 512.05; ¹H NMR (400 MHz, CDCl₃) δ 8.01 (s, 1H), 7.98 (d, J=6.0 Hz, 1H), 7.33 (d, J=9.2 Hz, 1H), 7.13 (s, 1H), 6.82 (s, 1H), 6.50-6.17 (m, 3H), 5.82-5.79 (m, 1H), 5.42-5.30 (m, 2H), 4.75-4.65 (m, 1H), 4.21-4.17 (m, 1H), 4.09-4.04 (m, 1H), 3.41-3.37 (m, 1H), 3.05 (s, 3H), 2.79-2.71 (m, 1H), 2.66-2.63 (m, 1H), 1.25-1.20 (m, 2H), 1.10-1.06 (m, 2H).

### Example 171: 3-[2-(1-Cyclopropyl-6,7-difluoro-1,3-benzodiazol-5-yl)ethynyl]-5-(methylamino)-1-[(3S)-1-(prop-2-enoyl)pyrrolidin-3-yl]pyrazole-4-carboxamide

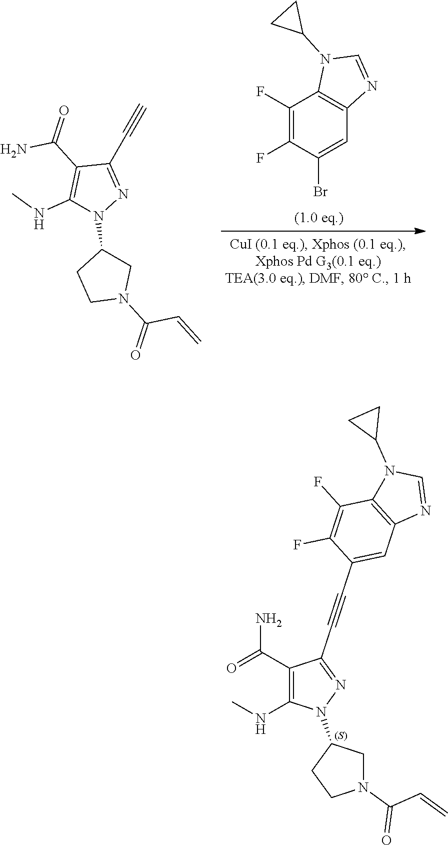

3-[2-(1-cyclopropyl-6,7-difluoro-1,3-benzodiazol-5-yl)ethynyl]-5-(methylamino)-1-[(3S)-1-(prop-2-enoyl)pyrrolidin-3-yl]pyrazole-4-carboxamide. MS ESI calculated for C₂₄H₂₃F₂N₇O₂ [M+H]⁺, 480.19, found 480.25; ¹H NMR (400 MHz, CDCl₃) δ 7.93 (d, J=1.6 Hz, 1H), 7.78-7.72 (m, 1H), 7.04 (s, 1H), 6.74-6.68 (m, 1H), 6.58-6.35 (m, 2H), 5.77-5.68 (m, 1H), 5.50 (s, 1H), 5.13-4.97 (m, 1H), 4.14-4.04 (m, 3H), 3.83-3.53 (m, 2H), 3.02 (t, J=5.6 Hz, 3H), 2.80-2.55 (m, 1H), 2.45-2.40 (m, 1H), 1.26-1.23 (m, 2H), 1.17-1.13 (m, 2H).

### Example 172: 3-((1-Cyclopropyl-6-fluoro-1H-benzo[d]imidazol-5-yl)ethynyl)-1-(1-(2-fluoroacryloyl)azetidin-3-yl)-5-(methylamino)-1H-pyrazole-4-carboxamide

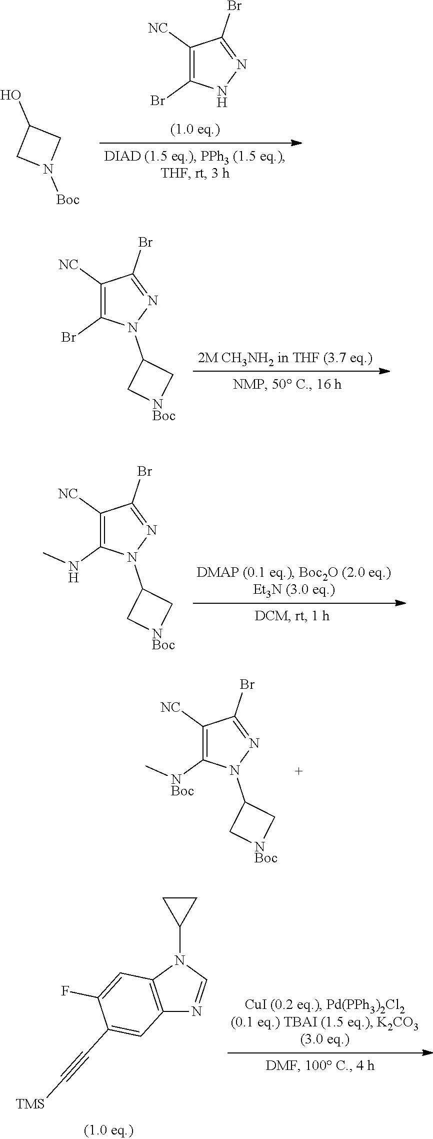 [SMILES: CC(C)(C)OC(=O)N1CC(O)C1 ; N#Cc1c(Br)n[nH]c1Br ; CC(C)(C)OC(=O)N1CC(n2nc(Br)c(C#N)c2Br)C1 ; CNc1c(C#N)c(Br)nn1C1CN(C(=O)OC(C)(C)C)C1 ; CN(C(=O)OC(C)(C)C)c1c(C#N)c(Br)nn1C1CN(C(=O)OC(C)(C)C)C1 ; C[Si](C)(C)C#Cc1cc2ncn(C3CC3)c2cc1F]

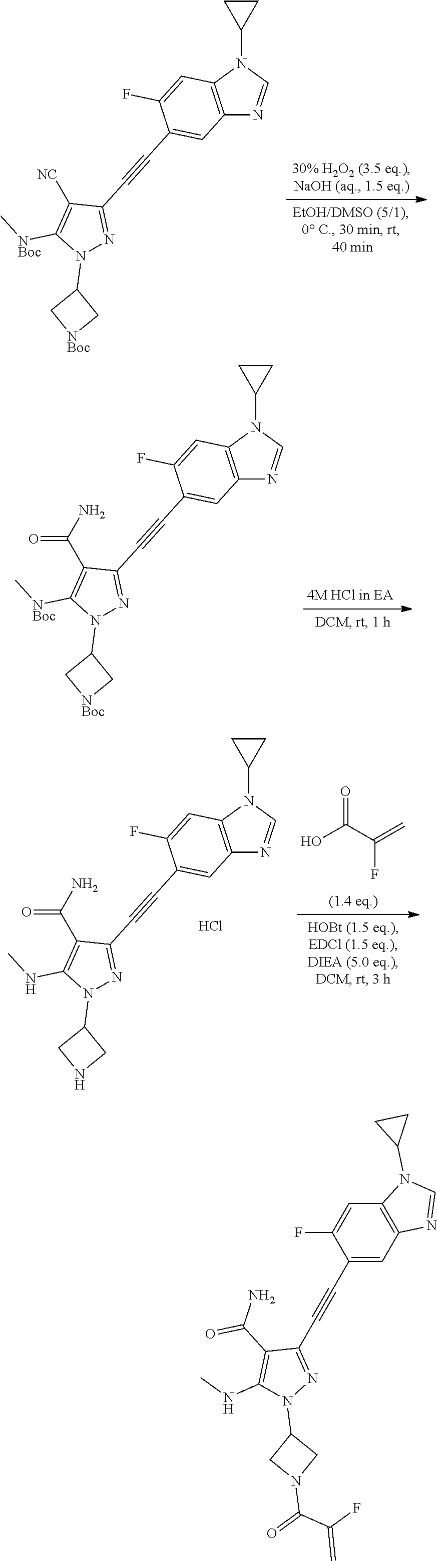

3-((1-cyclopropyl-6-fluoro-1H-benzo[d]imidazol-5-yl)ethynyl)-1-(1-(2-fluoroacryloyl)azetidin-3-yl)-5-(methylamino)-1H-pyrazole-4-carboxamide. MS ESI calculated for C₂₃H₂₁F₂N₇O₂ [M+H]⁺, 466.17, found 466.05; ¹H NMR (400 MHz, CDCl₃) δ 7.98 (d, J=4.7 Hz, 2H), 7.31 (d, J=9.1 Hz, 1H), 7.13 (s, 1H), 6.71 (q, J=6.0 Hz, 1H), 5.76-5.50 (m, 2H), 5.29-5.21 (m, 1H), 5.16-5.00 (m, 2H), 4.79 (s, 1H), 4.65 (dd, J=11.4, 5.8 Hz, 1H), 4.53 (t, J=9.7 Hz, 1H), 3.39-3.35 (m, 1H), 2.95 (d, J=5.8 Hz, 3H), 1.20 (d, J=7.2 Hz, 2H), 1.06 (s, 2H).

### Example 173: 3-((1-Cyclopropyl-4,6-difluoro-1H-benzo[d]imidazol-5-yl)ethynyl)-1-(1-(2-fluoroacryloyl)azetidin-3-yl)-5-(methylamino)-1H-pyrazole-4-carboxamide

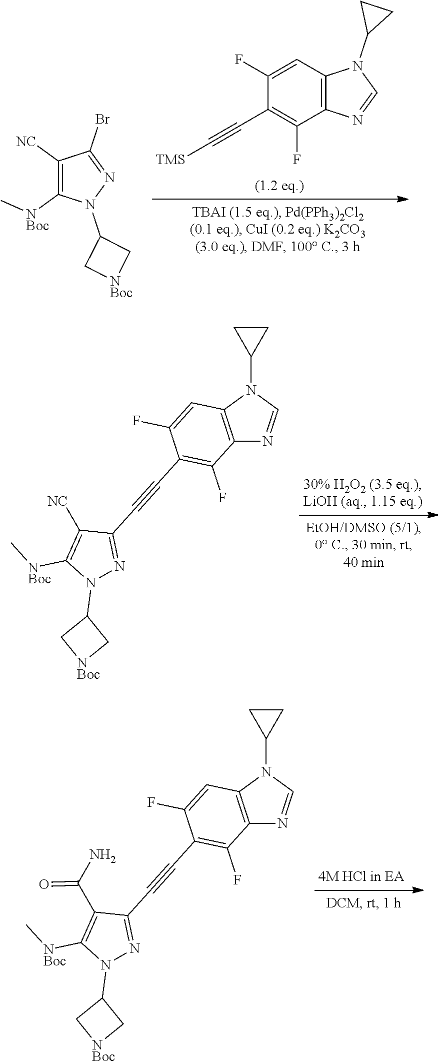 [SMILES: CN(C(=O)OC(C)(C)C)c1c(C#N)c(Br)nn1C1CN(C(=O)OC(C)(C)C)C1 ; C[Si](C)(C)C#Cc1c(F)cc2c(ncn2C2CC2)c1F ; CN(C(=O)OC(C)(C)C)c1c(C#N)c(C#Cc2c(F)cc3c(ncn3C3CC3)c2F)nn1C1CN(C(=O)OC(C)(C)C)C1 ; CN(C(=O)OC(C)(C)C)c1c(C(N)=O)c(C#Cc2c(F)cc3c(ncn3C3CC3)c2F)nn1C1CN(C(=O)OC(C)(C)C)C1]

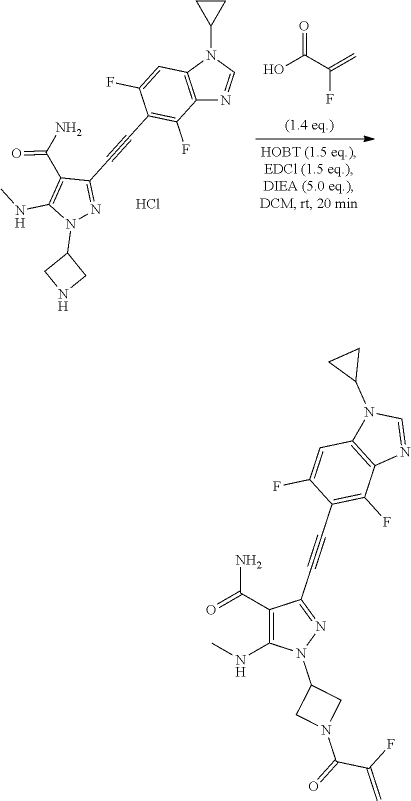

### Step 1: Tert-butyl 3-{5-[(tert-butoxycarbonyl)(methyl)amino]-4-cyano-3-[2-(1-cyclopropyl-4,6-difluoro-1,3-benzodiazol-5-yl)ethynyl]pyrazol-1-yl}azetidine-1-carboxylate

To a stirred solution of tert-butyl 3-{3-bromo-5-[(tert-butoxycarbonyl)(methyl)amino]-4-cyanopyrazol-1-yl}azetidine-1-carboxylate (1.50 g, 3.29 mmol) and 1-cyclopropyl-4,6-difluoro-5-[2-(trimethylsilyl)ethynyl]-1,3-benzodiazole (1.15 g, 3.94 mmol) in DMF (20.00 mL) were added K2CO3 (1.36 g, 9.86 mmol), TBAI (1.82 g, 4.93 mmol), CuI (0.13 g, 0.65 mmol) and Pd(PPh3)2Cl2 (0.23 g, 0.32 mmol) at room temperature under nitrogen atmosphere. The reaction mixture was degassed with nitrogen for three times and stirred for 3 h at 100° C. The resulting mixture was concentrated under reduced pressure. The residue was purified by silica gel column chromatography, eluted with 58% EA in PE. The fractions contained desired product were combined and concentrated to afford tert-butyl 3-{5-[(tert-butoxycarbonyl)(methyl)amino]-4-cyano-3-[2-(1-cyclopropyl-4,6-difluoro-1,3-benzodiazol-5-yl)ethynyl]pyrazol-1-yl}azetidine-1-carboxylate (1.20 g, 61%) as a light yellow solid. MS ESI calculated for C30H33F2N7O4 [M+H]+, 594.26, found 594.34; 1H NMR (400 MHz, CDCl3) δ 8.02 (s, 1H), 7.19 (d, J=8.0 Hz, 1H), 4.91 (s, 1H), 4.54 (s, 1H), 4.32 (t, J=7.8 Hz, 3H), 3.43-3.39 (m, 1H), 3.29 (s, 3H), 1.48 (s, 18H), 1.29-1.23 (m, 2H), 1.13-1.06 (m, 2H).

### Step 2: Tert-butyl 3-{5-[(tert-butoxycarbonyl)(methyl)amino]-4-carbamoyl-3-[2-(1-cyclopropyl-4,6-difluoro-1,3-benzodiazol-5-yl)ethynyl]pyrazol-1-yl}azetidine-1-carboxylate

To a stirred solution of tert-butyl 3-{5-[(tert-butoxycarbonyl)(methyl)amino]-4-cyano-3-[2-(1-cyclopropyl-4,6-difluoro-1,3-benzodiazol-5-yl)ethynyl]pyrazol-1-yl}azetidine-1-carboxylate (0.60 g, 1.01 mmol) and 30% H₂O₂ (0.36 mL, 3.53 mmol) in EtOH (10.00 mL) and DMSO (2.00 mL) was added LiOH (2.32 mL, 1.16 mmol) dropwise at 0° C. under nitrogen atmosphere. The reaction mixture was stirred for 30 min at 0° C. and 40 min at room temperature under nitrogen atmosphere. The resulting mixture was diluted with EA (10.00 mL). The residue was washed with water (3×5 mL). The combined organic layers were washed with brine (2×5 mL), dried over anhydrous Na2SO4. After filtration, the filtrate was concentrated under reduced pressure. The residue was purified by silica gel column chromatography, eluted with MeOH in DCM (0-3%). The fractions contained desired product were combined and concentrated to afford tert-butyl 3-{5-[(tert-butoxycarbonyl)(methyl)amino]-4-carbamoyl-3-[2-(1-cyclopropyl-4,6-difluoro-1,3-benzodiazol-5-yl)ethynyl]pyrazol-1-yl}azetidine-1-carboxylate (0.49 g, 78%) as a light yellow solid. MS ESI calculated for C30H35F2N7O5 [M+H]+, 612.27, found 612.35.

### Step 3: 1-(Azetidin-3-yl)-3-[2-(1-cyclopropyl-4,6-difluoro-1,3-benzodiazol-5-yl)ethynyl]-5-(methylamino)pyrazole-4-carboxamide hydrochloride

To a stirred solution of tert-butyl 3-{5-[(tert-butoxycarbonyl)(methyl)amino]-4-carbamoyl-3-[2-(1-cyclopropyl-4,6-difluoro-1,3-benzodiazol-5-yl)ethynyl]pyrazol-1-yl}azetidine-1-carboxylate (0.46 g, 0.79 mmol) in DCM (10.00 mL) was added HCl (4 M in EA) (10.00 mL) dropwise at 0° C. under nitrogen atmosphere. The reaction mixture was stirred for 1 h at room temperature under nitrogen atmosphere. The resulting mixture was concentrated under reduced pressure to afford 1-(azetidin-3-yl)-3-[2-(1-cyclopropyl-4,6-difluoro-1,3-benzodiazol-5-yl)ethynyl]-5-(methylamino)pyrazole-4-carboxamide hydrochloride (0.32 g, crude) as a light yellow solid. MS ESI calculated for C20H20ClF2N7O [M+H−HCl]+, 412.16, found 412.10.

### Step 4: 3-((1-Cyclopropyl-4,6-difluoro-1H-benzo[d]imidazol-5-yl)ethynyl)-1-(1-(2-fluoroacryloyl)azetidin-3-yl)-5-(methylamino)-1H-pyrazole-4-carboxamide

To a stirred solution of 1-(azetidin-3-yl)-3-[2-(1-cyclopropyl-4,6-difluoro-1,3-benzodiazol-5-yl)ethynyl]-5-(methylamino)pyrazole-4-carboxamide hydrochloride (0.33 g, 0.67 mmol) and 2-fluoroprop-2-enoic acid (85.38 mg, 0.94 mmol) in DCM (6.00 mL) was added HOBT (0.14 g, 1.02 mmol), EDCI (0.20 g, 1.02 mmol) and DIEA (0.43 g, 3.38 mmol) dropwise at 0° C. The reaction mixture was stirred for 20 min at room temperature. The residue was purified by silica gel column chromatography, eluted with MeOH in DCM (0-4%) to afford the crude product which was further purified by reverse phase flash with the following conditions: Column: Spherical C18, 20-40 μm, 40 g; Mobile Phase A: water (10 mmol/L NH4HCO3), Mobile Phase B: ACN; Flow rate: 60 mL/min; Gradient: 5B to 35B in 30 min; Detector: UV 254/210 nm. The fractions contained desired product were combined and concentrated to afford 3-((1-cyclopropyl-4,6-difluoro-1H-benzo[d]imidazol-5-yl)ethynyl)-1-(1-(2-fluoroacryloyl)azetidin-3-yl)-5-(methylamino)-1H-pyrazole-4-carboxamide (84.80 mg, 25%) as a white solid. MS ESI calculated for C23H20F3N7O2 [M+H]+, 484.16, found 484.15; 1H NMR (400 MHz, CDCl3) δ 7.97 (s, 1H), 7.19 (d, J=8.2 Hz, 1H), 7.10 (s, 1H), 6.72 (s, 1H), 5.74 (d, J=3.1 Hz, 1H), 5.67-5.40 (m, 1H), 5.33-5.26 (m, 1H), 5.17-4.98 (m, 2H), 4.81 (s, 1H), 4.68-4.66 (m, 1H), 4.55 (t, J=9.6 Hz, 1H), 3.40-3.36 (m, 1H), 2.96 (s, 3H), 1.30-1.20 (m, 2H), 1.15-1.06 (m, 2H).

### Example 174: 1-((3S,5R)-1-Acryloyl-5-(hydroxymethyl)pyrrolidin-3-yl)-3-((1-cyclopropyl-6-fluoro-1H-benzo[d]imidazol-5-yl)ethynyl)-5-(methylamino)-1H-pyrazole-4-carboxamide

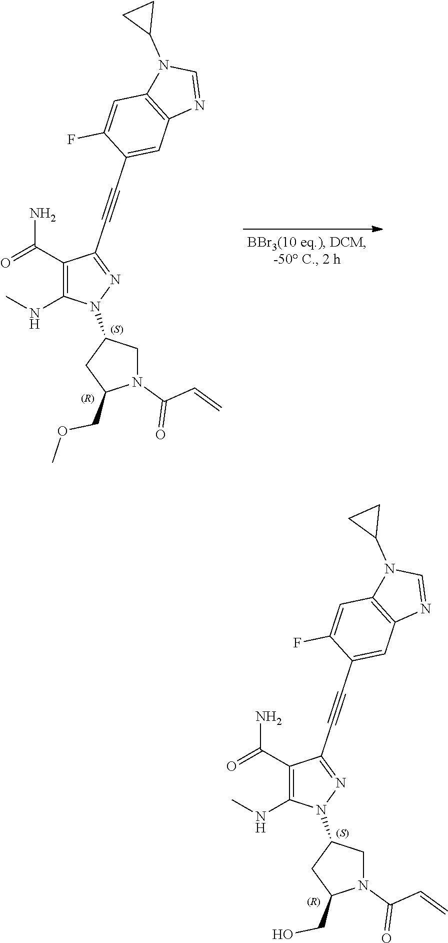

1-((3S,5R)-1-Acryloyl-5-(hydroxymethyl)pyrrolidin-3-yl)-3-((1-cyclopropyl-6-fluoro-1H-benzo[d]imidazol-5-yl)ethynyl)-5-(methylamino)-1H-pyrazole-4-carboxamide. MS ESI calculated for C₂₅H₂₆FN₇O₃[M+H]⁺, 492.21, found 492.10; ¹H NMR (400 MHz, DMSO-d₆) δ 8.37 (s, 1H), 7.96-7.76 (m, 1H), 7.65 (d, J=9.4 Hz, 1H), 7.50 (s, 1H), 6.77 (d, J=18.5 Hz, 1H), 6.73-6.53 (m, 2H), 6.17-5.95 (m, 1H), 5.69-5.36 (m, 1H), 5.30-5.08 (m, 1H), 5.04-4.83 (m, 1H), 4.34-4.19 (m, 1H), 4.09-3.74 (m, 2H), 3.71-3.44 (m, 3H), 2.96-2.72 (m, 3H), 2.63-2.57 (m, 1H), 2.40-2.26 (m, 1H), 1.14-1.08 (m, 2H), 1.08-1.02 (m, 2H).

### Example 175: Tert-butyl 3-[2-(1-cyclopropyl-6-fluoro-1,3-benzodiazol-5-yl)ethynyl]-5-(methylamino)-1-[1-(prop-2-enoyl)azetidin-3-yl]pyrazole-4-carboxamide

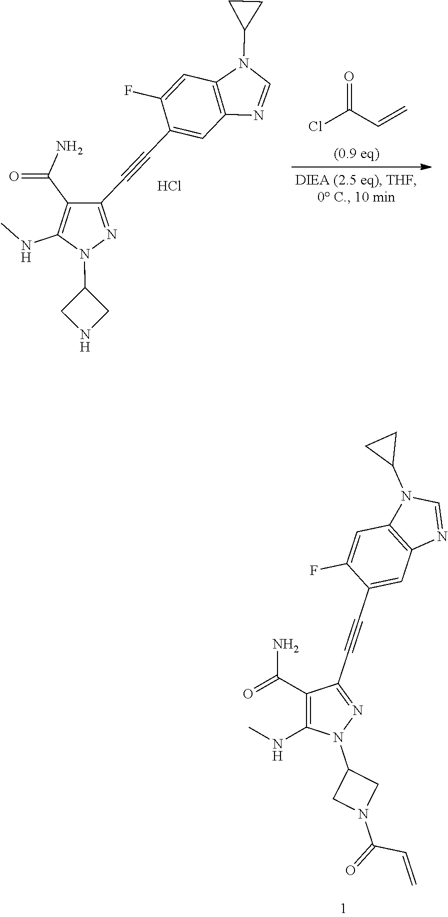

To a stirred mixture of 1-(azetidin-3-yl)-3-[2-(1-cyclopropyl-6-fluoro-1,3-benzodiazol-5-yl)ethynyl]-5-(methylamino)pyrazole-4-carboxamide hydrochloride (0.18 g, 0.42 mmol) and DIEA (0.13 g, 1.05 mmol) in THF (1.80 mL) was added acryloyl chloride (34.30 mg, 0.37 mmol) dropwise at 0° C. The reaction mixture was stirred for 10 min at 0° C. under nitrogen atmosphere. The resulting mixture was concentrated under reduced pressure. The residue was purified by silica gel column chromatography, eluted with DCM/MeOH (10/1) to afford crude product which was further purified by reverse phase flash with the following conditions: Column: YMC-Actus Triart C18 ExRS, 30 mm×150 mm, 5 μm; Mobile Phase A: water (10 mmol/L NH4HCO3), Mobile Phase B: ACN; Flow rate: 60 mL/min; Gradient: 25 B to 45 B in 8 min, 254 nm; RT1: 8 min. The fractions contained desired product were combined and concentrated to afford 3-[2-(1-cyclopropyl-6-fluoro-1,3-benzodiazol-5-yl)ethynyl]-5-(methylamino)-1-[1-(prop-2-enoyl)azetidin-3-yl]pyrazole-4-carboxamide (19.90 mg, 10%) as an off-white solid. MS ESI calculated for C23H22FN7O2 [M+H]+, 448.18, found 448.05; 1H NMR (300 MHz, CDCl3) δ 8.01 (d, J=5.3 Hz, 2H), 7.34 (d, J=8.9 Hz, 1H), 6.44-6.35 (m, 1H), 6.25-6.21 (m, 1H), 5.76-5.71 (m, 1H), 5.29 (s, 1H), 4.98 (s, 1H), 4.67-4.46 (m, 3H), 3.44-3.36 (m, 1H), 2.97 (d, J=5.2 Hz, 3H), 2.03 (s, 1H), 1.33-1.15 (m, 3H), 1.09 (s, 2H).

### Example 176: 1-((3S,5R)-1-Acryloyl-5-(fluoromethyl)pyrrolidin-3-yl)-3-((1-cyclopropyl-6-fluoro-1H-benzo[d]imidazol-5-yl)ethynyl)-5-(methylamino)-1H-pyrazole-4-carboxamide

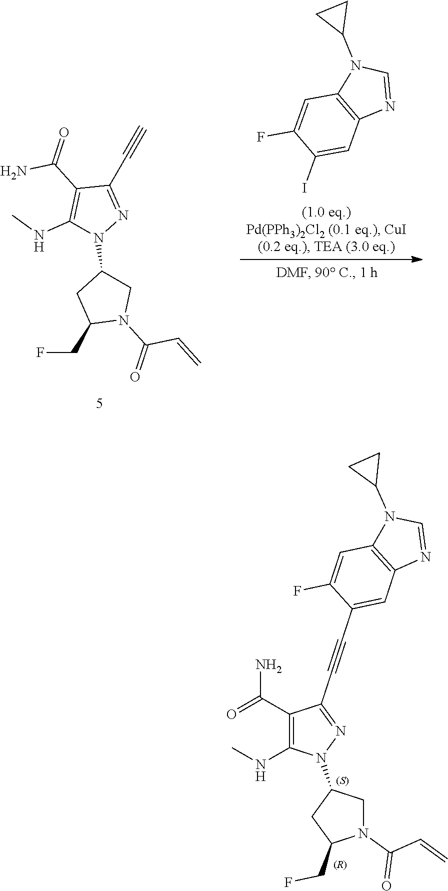

1-((3S,5R)-1-acryloyl-5-(fluoromethyl)pyrrolidin-3-yl)-3-((1-cyclopropyl-6-fluoro-1H-benzo[d]imidazol-5-yl)ethynyl)-5-(methylamino)-1H-pyrazole-4-carboxamide. MS ESI calculated for C₂₅H₂₅F₂N₇O₂[M+H]+, 494.20, found 494.45; ¹H NMR (400 MHz, DMSO-d₆) δ 8.56 (s, 1H), 7.72 (t, J=102.6 Hz, 3H), 7.14-6.42 (m, 3H), 6.23-6.14 (m, 1H), 5.71-5.64 (m, 1H), 5.26-5.18 (Cu, 1H), 4.89-4.34 (P, 3H), 4.17-3.67 (m, 2H), 3.51 (s, 1H), 2.96 (d, J=5.2 Hz, 3H), 2.79-2.64 (i, 1H), 2.39-2.37 (m, 1H), 1.20-0.95 (m, 4H).

### Example 177: 1-((3S,5R)-1-Acryloyl-5-(methoxymethyl)pyrrolidin-3-yl)-3-((4,6-difluoro-1H-benzo[d]imidazol-5-yl)ethynyl)-5-(methylamino)-1H-pyrazole-4-carboxamide

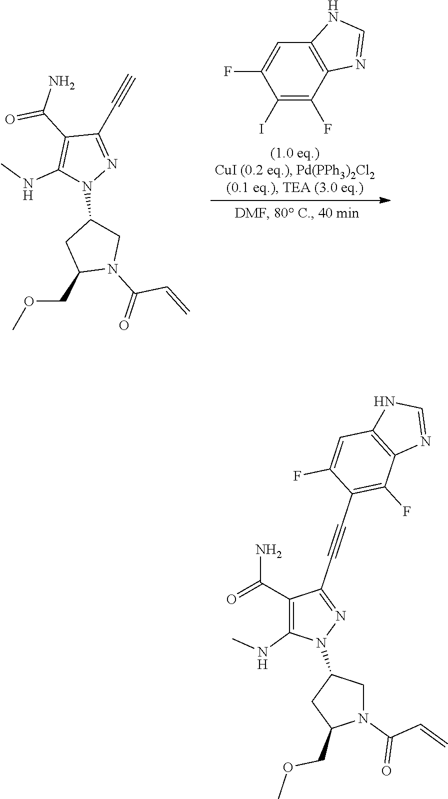 [SMILES: C#Cc1nn([C@H]2C[C@H](COC)N(C(=O)C=C)C2)c(NC)c1C(N)=O ; Fc1cc2[nH]cnc2c(F)c1I ; C=CC(=O)N1C[C@@H](n2nc(C#Cc3c(F)cc4[nH]cnc4c3F)c(C(N)=O)c2NC)C[C@@H]1COC]

1-((3S,5R)-1-acryloyl-5-(methoxymethyl)pyrrolidin-3-yl)-3-((4,6-difluoro-1H-benzo[d]imidazol-5-yl)ethynyl)-5-(methylamino)-1H-pyrazole-4-carboxamide. MS ESI calculated for C₂₃H₂₃F₂N₇O₃[M+H]+, 484.18, found 484.30; ¹H NMR (400 MHz, DMSO-d₆) δ 13.07 (s, 1H) 8.41 (s, 1H), 7.66-7.30 (m, 2H), 6.86-6.41 (m, 3H), 6.17-6.02 (m, 1H), 5.69-5.56 (m, 1H), 5.28-5.16 (m, 1H), 4.58-4.35 (m, 1H), 4.09-3.71 (m, 2H), 3.66-3.43 (m, 2H), 3.38-3.25 (m, 3H), 2.97-3.20 (m, 3H), 2.63-2.51 (m, 1H), 2.37-2.25 (m, 1H).

### Example 178: 1-((3S,5R)-1-Acryloyl-5-(fluoromethyl)pyrrolidin-3-yl)-3-((1-cyclopropyl-1H-benzo[d]imidazol-5-yl)ethynyl)-5-(methylamino)-1H-pyrazole-4-carboxamide

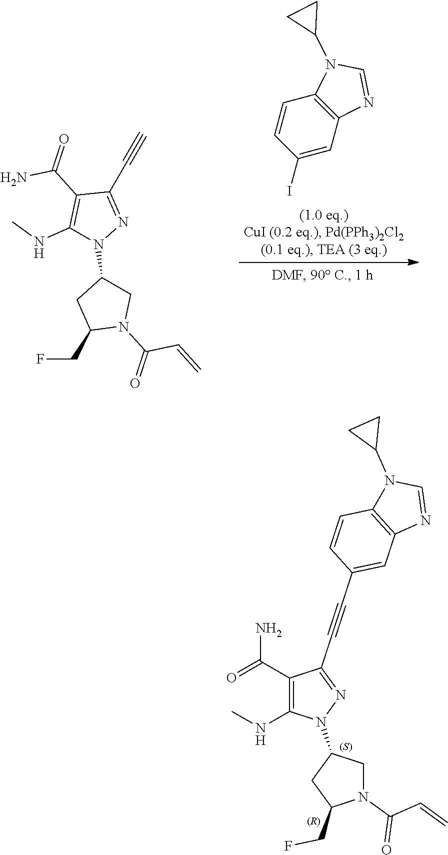

1-((3S,5R)-1-acryloyl-5-(fluoromethyl)pyrrolidin-3-yl)-3-((1-cyclopropyl-1H-benzo[d]imidazol-5-yl)ethynyl)-5-(methylamino)-1H-pyrazole-4-carboxamide. MS ESI calculated for C₂₅H₂₆FN₇O₂[M+H]+, 476.21, found 476.15; ¹H NMR (400 MHz, CDCl₃) δ 8.11 (s, 2H), 7.52 (d, J=4 Hz, 1H), 6.96 (s, 1H), 6.78 (s, 1H), 6.47-6.44 (m, 2H), 5.79-5.74 (m, 1H), 5.39-5.34 (m, 2H), 5.05-4.91 (m, 1H), 4.69-4.44 (m, 2H), 4.20-4.07 (m, 2H), 3.44 (s, 1H), 3.08-1.82 (m, 3H), 1.44-1.39 (m, 1H), 1.37-1.25 (m, 1H), 1.24 (d, J=4 Hz, 2H), 1.10 (s, 2H).

### Example 179: 1-((3S,5R)-1-Acryloyl-5-(fluoromethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide

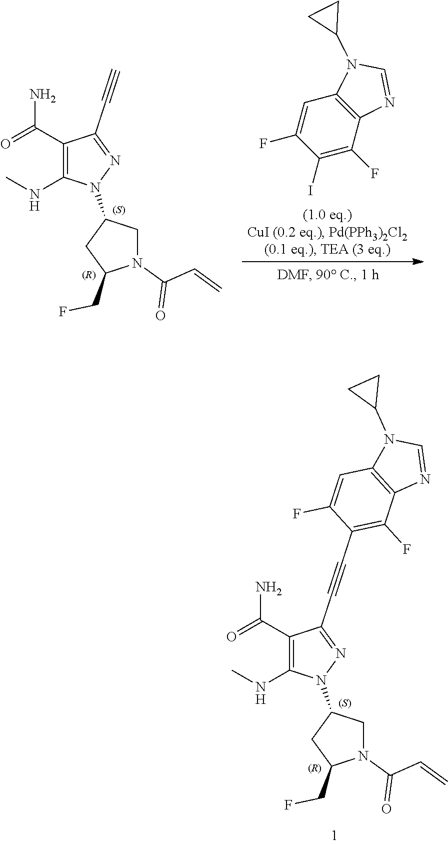

1-((3S,5R)-1-acryloyl-5-(fluoromethyl)pyrrolidin-3-yl)-3-((1-cyclopropyl-4,6-difluoro-1H-benzo[d]imidazol-5-yl)ethynyl)-5-(methylamino)-1H-pyrazole-4-carboxamide. MS ESI calculated for C₂₅H₂₄F₃N₇O₂[M+H]+, 512.19, found 512.35; ¹H NMR (400 MHz, CDCl₃) δ 8.00 (s, 1H), 7.19-7.08 (m, 2H), 6.50-6.41 (m, 2H), 5.86-5.74 (m, 1H), 5.41-5.19 (m, 2H), 5.06-4.90 (dd, J=4, 4 Hz, 1H), 4.68-4.44 (m, 2H), 4.19-4.14 (m, 1H), 4.14-4.06 (m, 1H), 3.43-3.73 (m, 1H), 3.06 (s, 3H), 2.90-2.82 (m, 1H), 2.44-2.39 (m, 1H), 1.27-1.23 (m, 2H), 1.12-1.07 (m, 2H).

### Example 180: 3-[2-(1-Cyclopropyl-6-fluoro-1,3-benzodiazol-5-yl)ethynyl]-1-[(3S,5S)-5-(fluoromethyl)-1-(prop-2-enoyl)pyrrolidin-3-yl]-5-(methylamino)pyrazole-4-carboxamide

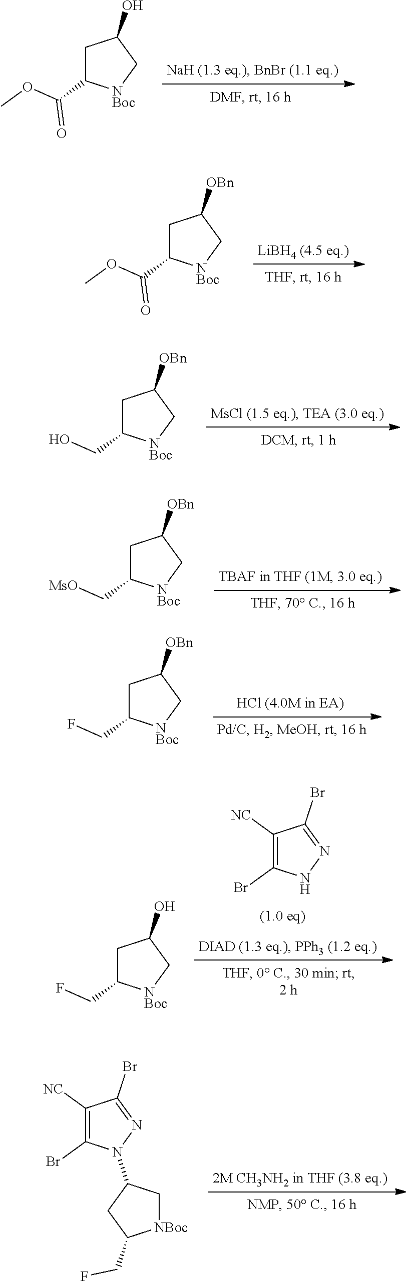

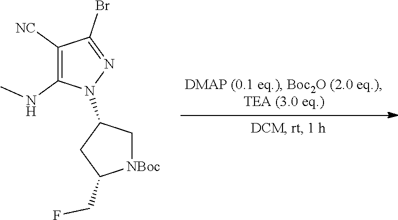 [SMILES: CNc1c(C#N)c(Br)nn1[C@H]1C[C@@H](CF)N(C(=O)OC(C)(C)C)C1]

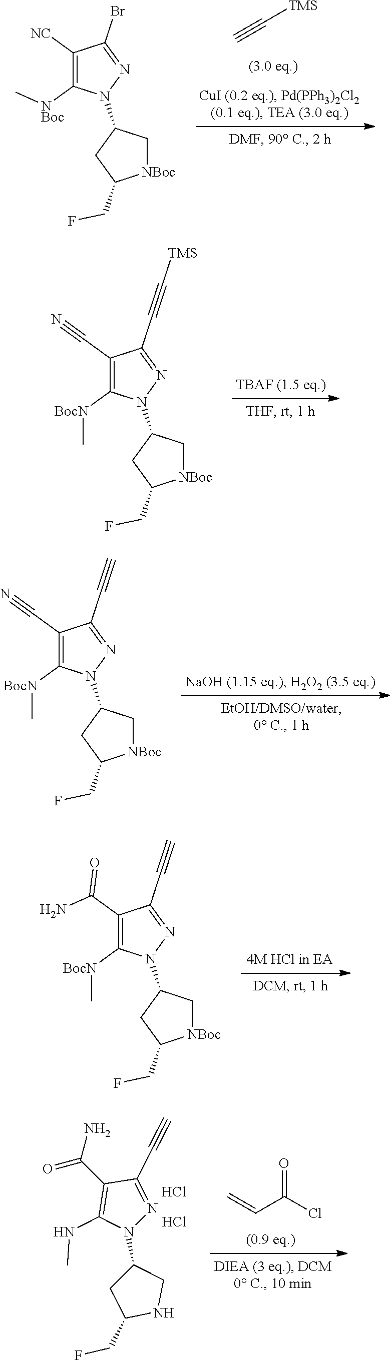

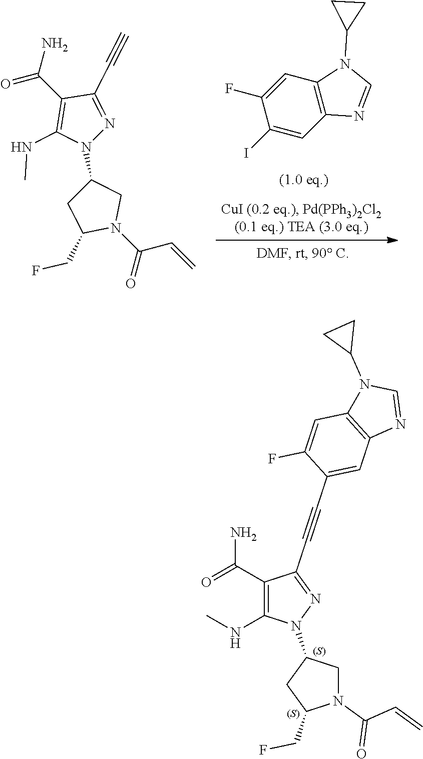

### Step 1: 1-(Tert-butyl) 2-methyl (2S,4R)-4-(benzyloxy)pyrrolidine-1,2-dicarboxylate

To a stirred solution of 1-(tert-butyl) 2-methyl (2S,4R)-4-hydroxypyrrolidine-1,2-dicarboxylate (30.00 g, 0.12 mol) and BnBr (23.01 g, 0.13 mol) in DMF (300.00 mL) was added NaH (60% in mineral oil) (6.36 g, 0.16 mol) in portions at 0° C. under nitrogen atmosphere. The reaction mixture was stirred for 16 h at room temperature under nitrogen atmosphere. The resulting mixture was diluted with water (100 mL) at 0° C. and extracted with EA (3×300 mL). The combined organic layers was washed with water (2×100 mL), dried over anhydrous Na2SO4. After filtration, the filtrate was concentrated under reduced pressure to afford 1-(tert-butyl) 2-methyl (2S,4R)-4-(benzyloxy)pyrrolidine-1,2-dicarboxylate (40.00 g, 97%) as a yellow oil. MS ESI calculated for C18H25NO5 [M+H]+, 336.17, found, 336.20.

### Step 2: Tert-butyl (2S,4R)-4-(benzyloxy)-2-(hydroxymethyl)pyrrolidine-1-carboxylate

To a stirred solution of 1-(tert-butyl) 2-methyl (2S,4R)-4-(benzyloxy)pyrrolidine-1,2-dicarboxylate (40.00 g, 0.12 mol) in THF (400.00 mL) was added LiBH4 (11.69 g, 0.53 mol) in portions at 0° C. under nitrogen atmosphere. The reaction mixture was stirred for 16 h at room temperature under nitrogen atmosphere. The resulting mixture was added water (200 mL) and quenched with NH4Cl (400 mL) at 0° C. The aqueous layers was extracted with EA (3×500 mL). The combined organic layers was washed with water (2×400 mL), dried over anhydrous Na2SO4. After filtration, the filtrate was concentrated under reduced pressure. The residue was purified by silica gel column chromatography, eluted with EA in PE (50-100%). The fractions contained desired product were combined and concentrated under reduced pressure to afford tert-butyl (2S,4R)-4-(benzyloxy)-2-(hydroxymethyl)pyrrolidine-1-carboxylate (23.00 g, 62%) as a yellow oil. MS ESI calculated for C17H25NO4 [M+H−100]+, 208.18, found 208.05; 1H NMR (400 MHz, DMSO-d6) δ 7.62-7.19 (m, 5H), 4.72-4.56 (m, 1H), 4.46 (d, J=4.3 Hz, 2H), 4.32-4.09 (m, 2H), 3.39-3.10 (m, 4H), 2.04 (d, J=19.8 Hz, 2H), 1.40 (s, 9H).

### Step 3: Tert-butyl (2S,4R)-4-(benzyloxy)-2-[(methanesulfonyloxy)methyl]pyrrolidine-1-carboxylate

To a stirred solution of tert-butyl (2S,4R)-4-(benzyloxy)-2-(hydroxymethyl)pyrrolidine-1-carboxylate (22.00 g, 71.57 mmol) and TEA (29.84 mL, 0.21 mol) in DCM (220.00 mL) was added MsCl (8.31 mL, 0.11 mol) in dropwise at 0° C. under nitrogen atmosphere. The reaction mixture was stirred for 1.5 h at room temperature under nitrogen atmosphere. The resulting mixture was diluted with water at 0° C. (100 mL). The aqueous layer was extracted with DCM (3×300 mL). The combined organic layers were washed with water (2×200 mL), dried over anhydrous Na2SO4. After filtration, the filtrate was concentrated under reduced pressure to afford tert-butyl (2S,4R)-4-(benzyloxy)-2-[(methanesulfonyloxy) methyl]pyrrolidine-1-carboxylate (27.00 g, 97%) as a yellow oil. MS ESI calculated for C18H27NO6S [M+H−100]+, 286.16, found 286.05.

### Step 4: Tert-butyl (2S,4R)-4-(benzyloxy)-2-(fluoromethyl)pyrrolidine-1-carboxylate

To a stirred solution of tert-butyl (2S,4R)-4-(benzyloxy)-2-[(methanesulfonyloxy) methyl]pyrrolidine-1-carboxylate (27.00 g, 70.04 mmol) in THF (270.00 mL) was added TBAF (54.94 g, 0.21 mol) dropwise at room temperature. The reaction mixture was stirred for 16 h at 70° C. The resulting mixture was cooled down and concentrated under reduced pressure. The residue was purified by silica gel column chromatography, eluted with EA in PE (0-50%). The fractions contained desired product were combined and concentrated under reduced pressure to afford tert-butyl (2S,4R)-4-(benzyloxy)-2-(fluoromethyl)pyrrolidine-1-carboxylate (18.00 g, 83%) as a white solid. MS ESI calculated for C17H24FNO3 [M+H−100]+, 210.17, found 210.20; 1H NMR (400 MHz, CDCl3) δ 7.42-7.35 (m, 1H), 7.35-7.28 (m, 4H), 4.70-4.65 (m, 3H), 4.48-4.33 (m, 1H), 4.16 (d, J=33.1 Hz, 2H), 3.67-3.39 (m, 2H), 2.28-2.12 (m, 2H), 1.49 (s, 9H).

### Step 5: Tert-butyl (2S,4R)-2-(fluoromethyl)-4-hydroxypyrrolidine-1-carboxylate

A stirred solution of tert-butyl (2S,4R)-4-(benzyloxy)-2-(fluoromethyl)pyrrolidine-1-carboxylate (18.00 g, 58.18 mmol) and Pd/C (10% wt in water) (3.71 g, 3.49 mmol) in MeOH (180.00 mL) was added 4 M HCl in EA (1.80 mL, 31.53 mmol) dropwise at room temperature. The reaction mixture was stirred for 16 h at room temperature under hydrogen atmosphere. The resulting mixture was filtered, the filter cake was washed with MeOH (2×100 mL). The filtrate was concentrated under reduced pressure. The residue was purified by silica gel column chromatography, eluted with EA in PE (30-80%). The fractions contained desired product were combined and concentrated under reduced pressure to afford tert-butyl (2S,4R)-2-(fluoromethyl)-4-hydroxypyrrolidine-1-carboxylate (12.00 g, 94%) as an off-white oil. MS ESI calculated for C10H18FNO3 [M+H−56]+, 164.13, found 164.05; 1H NMR (400 MHz, CDCl3) δ 4.75-4.35 (m, 3H), 4.13 (d, J=17.8 Hz, 1H), 3.48 (d, J=10.9 Hz, 2H), 2.25-2.00 (m, 3H), 1.49 (s, 9H).

### Step 6: Tert-butyl (2S,4S)-4-(3,5-dibromo-4-cyanopyrazol-1-yl)-2-(fluoromethyl)pyrrolidine-1-carboxylate

To a stirred solution of 3,5-dibromo-1H-pyrazole-4-carbonitrile (11.44 g, 45.61 mmol) and tert-butyl (2S,4R)-2-(fluoromethyl)-4-hydroxypyrrolidine-1-carboxylate (10.00 g, 45.60 mmol) in THF (100.00 mL) was added PPh3 (14.36 g, 54.73 mmol) at room temperature under nitrogen atmosphere. The reaction mixture was stirred for 30 min at room temperature under nitrogen atmosphere. To the above mixture was added DIAD (11.99 g, 59.29 mmol) dropwise at 0° C. The reaction mixture was stirred for another 2 h at room temperature. The resulting mixture was diluted with water (100 mL) at 0° C. and extracted with EA (3×300 mL). The combined organic layers was washed with water (2×100 mL), dried over anhydrous Na2SO4. After filtration, the filtrate was concentrated under reduced pressure to afford tert-butyl (2S,4S)-4-(3,5-dibromo-4-cyanopyrazol-1-yl)-2-(fluoromethyl)pyrrolidine-1-carboxylate (20.00 g, 97%) as an off-white oil. MS ESI calculated for C14H17Br2FN₄O₂ [M+H−56]+, 394.97, 396.97, 398.97, found 394.90, 396.90, 398.90; 1H NMR (400 MHz, CDCl3) δ 6.50-6.45 (m, 1H), 5.15-4.87 (m, 2H), 4.78-4.65 (m, 1H), 4.27-3.94 (m, 1H), 3.63 (s, 1H), 2.72-2.65 (m, 1H), 1.29 (s, 9H).

### Step 7: Tert-butyl (2S,4S)-4-[3-bromo-4-cyano-5-(methylamino)pyrazol-1-yl]-2-(fluoromethyl)pyrrolidine-1-carboxylate

To a stirred mixture of tert-butyl (2S,4S)-4-(3,5-dibromo-4-cyanopyrazol-1-yl)-2-(fluoromethyl)pyrrolidine-1-carboxylate (20.00 g, 44.23 mmol) in NMP (100.00 mL) was added CH3NH2 (1 M in THF) (84.05 mL, 0.17 mol). The reaction mixture was stirred for 16 h at 50° C. The residue was purified by reverse flash chromatography with the following conditions: column: C18 silica gel; mobile phase A: 10 mmol/L NH4HCO3 in water, mobile phase B: ACN, 20B to 80B gradient in 30 min; detector: UV 254 nm. The fractions contained desired product were combined and concentrated to afford tert-butyl (2S,4S)-4-[3-bromo-4-cyano-5-(methylamino)pyrazol-1-yl]-2-(fluoromethyl)pyrrolidine-1-carboxylate (15.00 g, 84%) as a yellow solid. MS ESI calculated for C15H21BrFN5O2 [M+H]+, 402.09, 404.09, found 402.15, 404.15; 1H NMR (400 MHz, CDCl3) δ 4.95-4.85 (m, 1H), 4.78-4.65 (m, 1H), 4.58-4.35 (m, 2H), 4.15-3.96 (m, 2H), 3.43 (d, J=14.1 Hz, 1H), 3.42-3.25 (m, 3H), 2.89-2.51 (m, 1H), 2.65-2.36 (m, 1H), 1.47 (s, 9H).

### Step 8: Tert-butyl (2S,4S)-4-{3-bromo-5-[(tert-butoxycarbonyl)(methyl)amino]-4-cyanopyrazol-1-yl}-2-(fluoromethyl)pyrrolidine-1-carboxylate

To a stirred solution of tert-butyl (2S,4S)-4-[3-bromo-4-cyano-5-(methylamino)pyrazol-1-yl]-2-(fluoromethyl)pyrrolidine-1-carboxylate (15.00 g, 37.29 mmol), Et3N (15.55 mL, 0.11 mol) and DMAP (0.46 g, 3.73 mmol) in DCM (150.00 mL) was added Boc₂O (16.28 g, 74.58 mmol) dropwise at 0° C. under nitrogen atmosphere. The reaction mixture was stirred for 1 h at room temperature. The resulting mixture was added water (100 mL) and extracted with DCM (3×300 mL). The combined organic layers was washed with water (2×200 mL), dried over anhydrous Na2SO4. After filtration, the filtrate was concentrated under reduced pressure to afford tert-butyl (2S,4S)-4-{3-bromo-5-[(tert-butoxycarbonyl)(methyl)amino]-4-cyanopyrazol-1-yl}-2-(fluoromethyl)pyrrolidine-1-carboxylate (16.00 g, 85%) as a white solid. MS ESI calculated for C20H29BrFN5O4 [M+H−112]+, 390.14, 392.14, found 389.95, 391.95; 1H NMR (400 MHz, CDCl3) δ 4.87-4.45 (m, 3H), 4.28-4.16 (m, 2H), 3.62-3.56 (m, 1H), 3.33-3.21 (m, 3H), 2.67-2.56 (m, 2H), 1.54 (s, 18H).

### Step 9: Tert-butyl (2S,4S)-4-{5-[(tert-butoxycarbonyl)(methyl)amino]-4-cyano-3-[2-(trimethylsilyl)ethynyl]pyrazol-1-yl}-2-(fluoromethyl)pyrrolidine-1-carboxylate

To a stirred solution of tert-butyl (2S,4S)-4-{3-bromo-5-[(tert-butoxycarbonyl)(methyl)amino]-4-cyanopyrazol-1-yl}-2-(fluoromethyl)pyrrolidine-1-carboxylate (8.00 g, 15.92 mmol), trimethylsilylacetylene (4.69 g, 47.77 mmol), Pd(PPh3)2Cl2 (1.12 g, 1.59 mmol) and CuI (0.61 g, 3.18 mmol) in DMF (80.00 mL) was added TEA (6.64 mL, 47.77 mmol). The reaction mixture was degassed with argon for three times and stirred for 2 h at 90° C. The resulting mixture was cooled and concentrated under vacuum. The residue was purified by silica gel column chromatography, eluted with EA in PE (10-50%). The fractions contained desired product were combined and concentrated to afford tert-butyl (2S,4S)-4-{5-[(tert-butoxycarbonyl)(methyl)amino]-4-cyano-3-[2-(trimethylsilyl)ethynyl]pyrazol-1-yl}-2-(fluoromethyl)pyrrolidine-1-carboxylate (7.40 g, 89%) as a white solid. MS ESI calculated for C25H38FN5O4Si [M+H]+, 520.27, found 520.45; 1H NMR (400 MHz, CDCl3) δ 4.74-4.51 (m, 3H), 4.42-4.91 (m, 2H), 3.61-3.49 (m, 1H), 3.24 (s, 3H), 2.53 (d, J=36.9 Hz, 2H), 1.47 (s, 18H), 0.30 (s, 9H).

### Step 10: Tert-butyl (2S,4S)-4-{5-[(tert-butoxycarbonyl)(methyl)amino]-4-cyano-3-ethynylpyrazol-1-yl}-2-(fluoromethyl)pyrrolidine-1-carboxylate

To a stirred solution of tert-butyl (2S,4S)-4-{5-[(tert-butoxycarbonyl)(methyl)amino]-4-cyano-3-[2-(trimethylsilyl)ethynyl]pyrazol-1-yl}-2-(fluoromethyl)pyrrolidine-1-carboxylate (7.40 g, 14.24 mmol) in THF (74.00 mL) was added TBAF (5.58 mL, 21.36 mmol) dropwise at 0° C. The reaction mixture was stirred for 1 h at room temperature. The residue was purified by silica gel column chromatography, eluted with EA in PE (20-90%). The fractions contained desired product were combined and concentrated to afford tert-butyl (2S,4S)-4-{5-[(tert-butoxycarbonyl)(methyl)amino]-4-cyano-3-ethynylpyrazol-1-yl}-2-(fluoromethyl)pyrrolidine-1-carboxylate (4.70 g, 73%) as a yellow solid. MS ESI calculated for C22H30FN5O [M+H]+, 448.23, found 448.20.

### Step 11: Tert-butyl (2S,4S)-4-{5-[(tert-butoxycarbonyl)(methyl)amino]-4-carbamoyl-3-ethynylpyrazol-1-yl}-2-(fluoromethyl)pyrrolidine-1-carboxylate

To a stirred solution of tert-butyl (2S,4S)-4-{5-[(tert-butoxycarbonyl)(methyl)amino]-4-cyano-3-ethynylpyrazol-1-yl}-2-(fluoromethyl)pyrrolidine-1-carboxylate (2.50 g, 5.58 mmol) and NaOH (1 M in water) (6.42 mL, 6.42 mmol) in DMSO (2.50 mL) and EtOH (25.00 mL) was added H₂O₂ (30% in water) (0.46 mL, 19.55 mmol) dropwise at 0° C. The reaction mixture was stirred for 1 h at 0° C. The resulting mixture was diluted with water (25 mL) and extracted with EA (3×50 mL). The combined organic layers was washed with water (2×25 mL), dried over anhydrous Na2SO4. After filtration, the filtrate was concentrated under reduced pressure. The residue was purified by silica gel column chromatography, eluted with EA in PE (50-100%). The fractions contained desired product were combined and concentrated to afford tert-butyl (2S,4S)-4-{5-[(tert-butoxycarbonyl)(methyl)amino]-4-carbamoyl-3-ethynylpyrazol-1-yl}-2-(fluoromethyl)pyrrolidine-1-carboxylate (2.40 g, 92%) as a white solid. MS ESI calculated for C22H32FN5O5 [M+H]+, 466.24, found, 466.20.

### Step 12: 3-Ethynyl-1-[(3S,5S)-5-(fluoromethyl)pyrrolidin-3-yl]-5-(methylamino)pyrazole-4-carboxamide dihydrochloride

To a stirred solution of tert-butyl (2S,4S)-4-{5-[(tert-butoxycarbonyl)(methyl)amino]-4-carbamoyl-3-ethynylpyrazol-1-yl}-2-(fluoromethyl)pyrrolidine-1-carboxylate (2.30 g, 4.94 mmol) in DCM (10.00 mL) was added HCl (4 M in EA) (10.00 mL, 0.17 mol) dropwise at 0° C. The reaction mixture was stirred for 1 h at room temperature. The resulting mixture was concentrated under reduced pressure to afford 3-ethynyl-1-[(3S,5S)-5-(fluoromethyl)pyrrolidin-3-yl]-5-(methylamino)pyrazole-4-carboxamide dihydrochloride (1.48 g, crude) as a yellow solid. MS ESI calculated for C12H16FN5O [M+H−2 HCl]+, 266.13, found 266.15.

### Step 13: 3-Ethynyl-1-[(3S,5S)-5-(fluoromethyl)-1-(prop-2-enoyl)pyrrolidin-3-yl]-5-(methylamino)pyrazole-4-carboxamide

To a stirred solution of 3-ethynyl-1-[(3S,5S)-5-(fluoromethyl)pyrrolidin-3-yl]-5-(methylamino)pyrazole-4-carboxamide dihydrochloride (1.48 g, 4.90 mmol) and DIEA (2.56 mL, 14.71 mmol) in DCM (23.00 mL) was added acryloyl chloride (0.40 g, 4.42 mmol) dropwise at 0° C. under nitrogen atmosphere. The reaction mixture was stirred for 10 min at 0° C. The resulting mixture was added water (20 mL) and extracted with EA (3×50 mL). The combined organic layers was washed with brine (1×20 mL), dried over anhydrous Na2SO4. After filtration, the filtrate was concentrated under reduced pressure. The residue was purified by silica gel column chromatography, eluted with MeOH in DCM (0-5%). The fractions contained desired product were combined and concentrated to afford 3-ethynyl-1-[(3S,5S)-5-(fluoromethyl)-1-(prop-2-enoyl)pyrrolidin-3-yl]-5-(methylamino)pyrazole-4-carboxamide (1.30 g, 83%) as a yellow solid. MS ESI calculated for C15H18FN5O2 [M+H]+, 320.14, found 320.10.

### Step 14: 3-[2-(1-Cyclopropyl-6-fluoro-1,3-benzodiazol-5-yl)ethynyl]-1-[(3S,5S)-5-(fluoromethyl)-1-(prop-2-enoyl)pyrrolidin-3-yl]-5-(methylamino)pyrazole-4-carboxamide

To a stirred mixture of 3-ethynyl-1-[(3S,5S)-5-(fluoromethyl)-1-(prop-2-enoyl)pyrrolidin-3-yl]-5-(methylamino)pyrazole-4-carboxamide (0.30 g, 0.94 mmol), 1-cyclopropyl-6-fluoro-5-iodo-1,3-benzodiazole (0.29 g, 0.94 mmol), CuI (35.78 mg, 0.18 mmol) and Pd(PPh3)2Cl2 (65.94 mg, 0.09 mmol) in DMF (9.30 mL) was added TEA (0.28 g, 2.77 mmol). The reaction mixture was degassed with nitrogen for three times and stirred for 1 h at 90° C. The resulting solution was concentrated under reduced pressure. The residue was purified by silica gel column chromatography, eluted with CH2Cl2/MeOH (10/1) to afford crude product which was further purified by reverse flash chromatography with the following conditions: column: C18 silica gel; mobile phase: ACN in water (10 mmol/L NH4HCO3), 5% to 70% gradient in 30 min; detector: UV 254 nm. The fractions contained desired product were combined and concentrated to afford 3-[2-(1-cyclopropyl-6-fluoro-1,3-benzodiazol-5-yl)ethynyl]-1-[(3S,5S)-5-(fluoromethyl)-1-(prop-2-enoyl)pyrrolidin-3-yl]-5-(methylamino)pyrazole-4-carboxamide (95.90 mg, 20%) as an off-white solid. MS ESI calculated for C25H25F2N7O2 [M+H]+, 494.21, found 494.35; 1H NMR (400 MHz, CDCl3) δ 8.01 (d, J=6.7 Hz, 2H), 7.34 (d, J=8.9 Hz, 1H), 7.15 (s, 1H), 6.68 (s, 1H), 6.43 (d, J=8.4 Hz, 2H), 5.77 (m, 1H), 5.39 (s, 1H), 4.96-4.87 (m, 1H), 4.82 (d, J=23.0 Hz, 1H), 4.77-4.60 (m, 1H), 4.51 (s, 1H), 4.21-4.07 (m, 1H), 3.43-3.37 (m, 1H), 3.01 (s, 3H), 2.81 (q, J=10.3 Hz, 1H), 2.59 (s, 1H), 1.30-1.16 (m, 2H), 1.09 (q, J=3.9, 3.4 Hz, 2H).

### Example 181: 3-[2-(1-Cyclopropyl-4,6-difluoro-1,3-benzodiazol-5-yl)ethynyl]-1-[(3S,5S)-5-(fluoromethyl)-1-(prop-2-enoyl)pyrrolidin-3-yl]-5-(methylamino)pyrazole-4-carboxamide

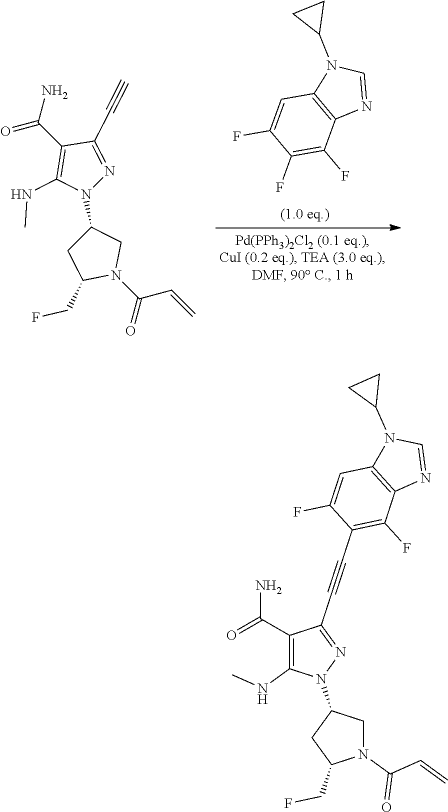 [SMILES: C#Cc1nn([C@H]2C[C@@H](CF)N(C(=O)C=C)C2)c(NC)c1C(N)=O ; Fc1cc2c(ncn2C2CC2)c(F)c1F ; C=CC(=O)N1C[C@@H](n2nc(C#Cc3c(F)cc4c(ncn4C4CC4)c3F)c(C(N)=O)c2NC)C[C@H]1CF]

To a stirred solution of 3-ethynyl-1-[(3S,5S)-5-(fluoromethyl)-1-(prop-2-enoyl)pyrrolidin-3-yl]-5-(methylamino)pyrazole-4-carboxamide (0.25 g, 0.78 mmol), 1-cyclopropyl-4,6-difluoro-5-iodo-1,3-benzodiazole (0.25 g, 0.78 mmol), CuI (29.82 mg, 0.16 mmol) and Pd(PPh3)2Cl2 (54.95 mg, 0.08 mmol) in DMF (2.50 mL, 32.30 mmol) was added TEA (0.24 g, 2.35 mmol). The reaction mixture was degassed with nitrogen for three times and stirred for 1 h at 90° C. The resulting mixture was concentrated under reduced pressure. The residue was purified by silica gel column chromatography, eluted with DCM/MeOH (10/1) to afford the crude product which was further purified by reverse flash chromatography with the following conditions: column: C18 silica gel; mobile phase: ACN in water, 5% to 35% gradient in 40 min; detector: UV 254 nm. The fractions contained desired product were combined and concentrated to afford 3-[2-(1-cyclopropyl-4,6-difluoro-1,3-benzodiazol-5-yl)ethynyl]-1-[(3S,5S)-5-(fluoromethyl)-1-(prop-2-enoyl)pyrrolidin-3-yl]-5-(methylamino)pyrazole-4-carboxamide (91.70 mg, 22%) as a white solid. MS ESI calculated for C25H24F3N7O2 [M+H]+, 512.19, found 512.10; 1H NMR (400 MHz, CDCl3) δ 7.98 (s, 1H), 7.19 (d, J=8.3 Hz, 1H), 7.10 (s, 1H), 6.65-6.36 (m, 2H), 5.79-5.73 (m, 1H), 5.43 (s, 1H), 5.00-4.59 (m, 3H), 4.58-4.44 (m, 1H), 4.12-4.07 (m, 1H), 3.42-3.38 (m, 1H), 3.01 (s, 3H), 2.87-2.44 (m, 2H), 1.30-1.21 (m, 2H), 1.21-1.05 (m, 2H).

### Example 182: 3-((1-Cyclopropyl-6-fluoro-1H-benzo[d]imidazol-5-yl)ethynyl)-1-((3S,5R)-5-(methoxymethyl)-1-propioloylpyrrolidin-3-yl)-5-(methylamino)-1H-pyrazole-4-carboxamide

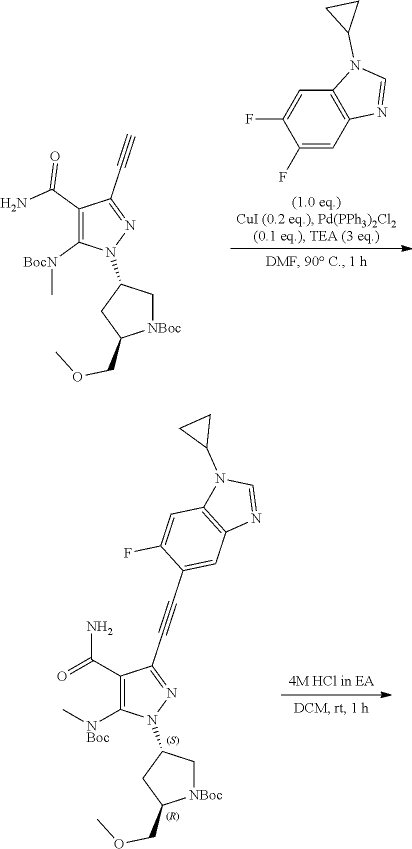

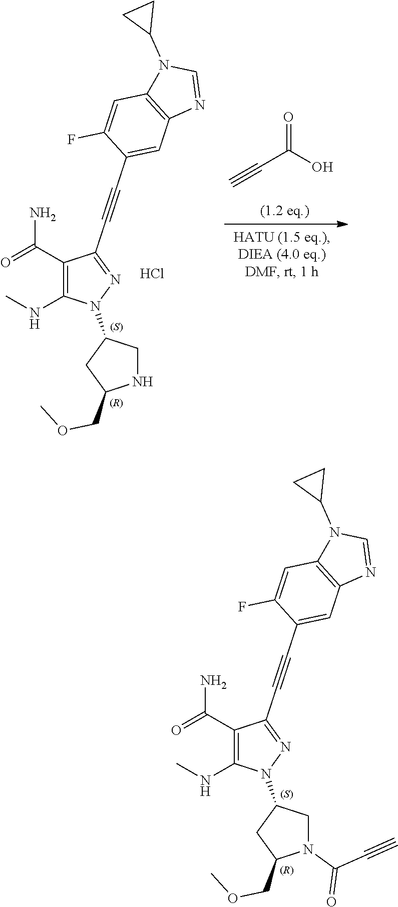

Step 1: Tert-butyl (2R,4S)-4-(5-((tert-butoxycarbonyl)(methyl)amino)-4-carbamoyl-3-((1-cyclopropyl-6-fluoro-1H-benzo[d]imidazol-5-yl)ethynyl)-1H-pyrazol-1-yl)-2-(methoxymethyl)pyrrolidine-1-carboxylate

To a stirred mixture of tert-butyl (2R,4S)-4-{5-[(tert-butoxycarbonyl)(methyl)amino]-4-carbamoyl-3-ethynylpyrazol-1-yl}-2-(methoxymethyl)pyrrolidine-1-carboxylate (2.00 g, 4.18 mmol), CuI (0.16 g, 0.83 mmol), Pd(PPh₃)₂Cl₂ (0.29 g, 0.41 mmol) and 1-cyclopropyl-6-fluoro-5-iodo-1,3-benzodiazole (1.27 g, 4.18 mmol) in DMF (20.00 mL) was added TEA (1.75 mL, 12.56 mmol). The reaction mixture was degassed with nitrogen for three times and stirred for 1 h at 90° C. The resulting mixture was allowed to cool down to room temperature and concentrated under reduced pressure. The residue was purified by silica gel column chromatography, eluted with DCM/MeOH (10/1). The fractions contained desired product were combined and concentrated to afford tert-butyl (2R,4S)-4-(5-((tert-butoxycarbonyl)(methyl)amino)-4-carbamoyl-3-((1-cyclopropyl-6-fluoro-1H-benzo[d]imidazol-5-yl)ethynyl)-1H-pyrazol-1-yl)-2-(methoxymethyl)pyrrolidine-1-carboxylate (2.20 g, 80%) as an off-white solid. MS ESI calculated for C₃₃H₄₂FN₇O₆[M+H]⁺, 652.32, found 652.85; ¹H NMR (400 MHz, CDCl₃) δ 10.42 (s, 1H), 8.11-7.91 (m, 2H), 7.33 (d, J=8.9 Hz, 1H), 5.52 (s, 1H), 5.07 (s, 1H), 4.23 (s, 1H), 3.90-3.50 (m, 3H), 3.38 (s, 1H), 3.19-3.12 (m, 6H), 2.66 (s, 1H), 2.29 (s, 1H), 1.82 (s, 1H), 1.48 (t, J=7.4 Hz, 18H), 1.28-1.17 (m, 2H), 1.11-0.79 (m, 2H).

### Step 2: 3-((1-Cyclopropyl-6-fluoro-1H-benzo[d]imidazol-5-yl)ethynyl)-1-((3S,5R)-5-(methoxymethyl)pyrrolidin-3-yl)-5-(methylamino)-1H-pyrazole-4-carboxamide hydrochloride

To a stirred mixture of tert-butyl (2R,4S)-4-(5-((tert-butoxycarbonyl)(methyl)amino)-4-carbamoyl-3-((1-cyclopropyl-6-fluoro-1H-benzo[d]imidazol-5-yl)ethynyl)-1H-pyrazol-1-yl)-2-(methoxymethyl)pyrrolidine-1-carboxylate (2.20 g, 3.37 mmol) in DCM (11.00 mL) was added 4 M HCl in EA (22.00 mL) dropwise at 0° C. under nitrogen atmosphere. The reaction mixture was stirred for 1 h at room temperature under nitrogen atmosphere. The precipitated solids were collected by filtration and washed with DCM (3×20 mL). The crude product 3-((1-cyclopropyl-6-fluoro-1H-benzo[d]imidazol-5-yl)ethynyl)-1-((3S,5R)-5-(methoxymethyl)pyrrolidin-3-yl)-5-(methylamino)-1H-pyrazole-4-carboxamide hydrochloride (1.60 g, crude) as an off-white was used in the next step directly without further purification. MS ESI calculated for C₂₃H₂₇ClFN₇O₂ [M+H−HCl]⁺, 452.21, found 452.30.

### Step 3: 3-((1-Cyclopropyl-6-fluoro-1H-benzo[d]imidazol-5-yl)ethynyl)-1-((3S,5R)-5-(methoxymethyl)-1-propioloylpyrrolidin-3-yl)-5-(methylamino)-1H-pyrazole-4-carboxamide

To a stirred mixture of 3-((1-cyclopropyl-6-fluoro-1H-benzo[d]imidazol-5-yl)ethynyl)-1-((3S,5R)-5-(methoxymethyl)pyrrolidin-3-yl)-5-(methylamino)-1H-pyrazole-4-carboxamide hydrochloride (0.30 g, 0.61 mmol) and HATU (0.35 g, 0.92 mmol) in DMF (3.00 mL) were added DIEA (0.43 mL, 2.46 mmol) and propiolic acid (50.85 mg, 0.73 mmol) dropwise at 0° C. under nitrogen atmosphere. The reaction mixture was stirred for 1 h at room temperature under nitrogen atmosphere. The resulting mixture was concentrated under reduced pressure. The residue was purified by silica gel column chromatography, eluted with DCM/MeOH (10/1) to afford crude product which was further purified by reverse flash chromatography with the following conditions: column: C18 silica gel; mobile phase: ACN in water (10 mmol/L NH₄HCO₃), 10% to 50% gradient in 30 min; detector: UV 254 nm. The fractions contained desired product were combined and concentrated to afford 3-((1-cyclopropyl-6-fluoro-1H-benzo[d]imidazol-5-yl)ethynyl)-1-((3S,5R)-5-(methoxymethyl)-1-propioloylpyrrolidin-3-yl)-5-(methylamino)-1H-pyrazole-4-carboxamide (71.50 mg, 23%) as an off-white solid. MS ESI calculated for C₂₆H₂₆FN₇O₃[M+H]⁺, 504.21, found 504.20; ¹H NMR (400 MHz, CDCl₃) δ 8.01 (s, 2H), 7.36 (s, 1H), 7.11 (s, 1H), 6.80 (s, 1H), 5.50-5.32 (m, 2H), 4.51-4.47 (m, 1H), 4.25-4.06 (m, 1H), 4.05-3.84 (m, 1H), 3.69-3.63 (m, 1H), 3.44-3.35 (m, 5H), 3.10-2.99 (m, 4H), 2.97-2.69 (m, 1H), 2.39-2.35 (m, 1H), 1.21-1.19 (m, 2H), 1.06 (s, 2H).

### Example 183: 1-((3S,5R)-1-(But-2-ynoyl)-5-(methoxymethyl)pyrrolidin-3-yl)-3-((1-cyclopropyl-6-fluoro-1H-benzo[d]imidazol-5-yl)ethynyl)-5-(methylamino)-1H-pyrazole-4-carboxamide

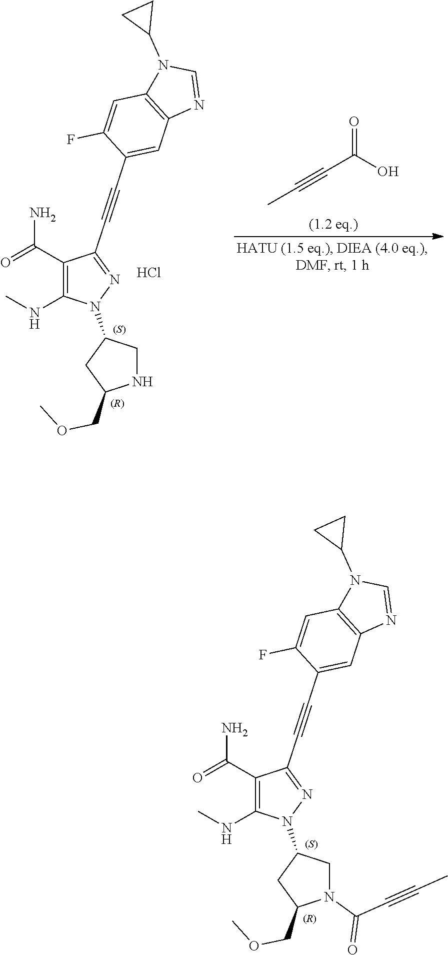

1-((3S,5R)-1-(But-2-ynoyl)-5-(methoxymethyl)pyrrolidin-3-yl)-3-((1-cyclopropyl-6-fluoro-1H-benzo[d]imidazol-5-yl)ethynyl)-5-(methylamino)-1H-pyrazole-4-carboxamide. MS ESI calculated for C₂₇H₂₈FN₇O₃[M+H]⁺, 518.22, found 518.30; ¹H NMR (400 MHz, CDCl₃) δ 8.02-7.93 (m, 2H), 7.31-7.26 (m, 1H), 7.11 (s, 1H), 6.80 (s, 1H), 5.49-5.31 (m, 2H), 4.49-4.43 (m, 1H), 4.21-4.02 (m, 1H), 4.02-3.81 (m, 1H), 3.65-3.62 (m, 1H), 3.43-3.32 (m, 5H), 3.02 (d, J=16.6 Hz, 3H), 2.93-2.66 (m, 1H), 2.36-2.33 (m, 1H), 2.02 (s, 1H), 1.98 (s, 2H), 1.21-1.18 (m, 2H), 1.15-1.02 (m, 2H).

### Example 184: 3-[2-(1-Cyclopropyl-4,6-difluoro-1,3-benzodiazol-5-yl)ethynyl]-1-[(3 S,5R)-5-(methoxymethyl)-1-(prop-2-ynoyl)pyrrolidin-3-yl]-5-(methylamino)pyrazole-4-carboxamide

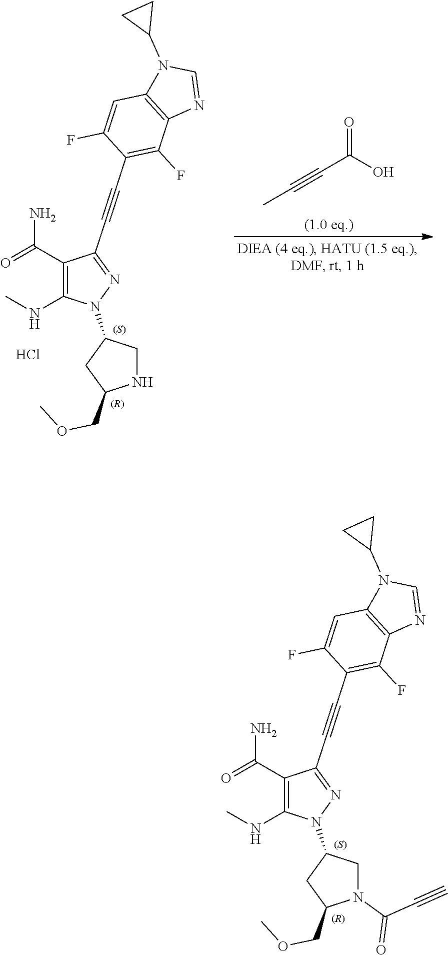

3-[2-(1-cyclopropyl-4,6-difluoro-1,3-benzodiazol-5-yl)ethynyl]-1-[(3S,5R)-5-(methoxymethyl)-1-(prop-2-ynoyl)pyrrolidin-3-yl]-5-(methylamino)pyrazole-4-carboxamide. MS ESI calculated for C₂₆H₂₅F₂N₇O₃[M+H]+, 522.20, found 522.35; ¹H NMR (400 MHz, CDCl₃) δ 7.96 (d, J=4 Hz, 1H), 7.19-7.16 (m, 1H), 7.08 (s, 1H), 6.85-6.73 (m, 1H), 5.49-5.35 (m, 2H), 4.59-4.48 (m, 1H), 4.26-4.21 (m, 1H), 4.07-4.02 (m, 1H), 3.80-3.78 (m, 1H), 3.60-3.57 (m, 1H), 3.44-3.36 (m, 4H), 3.09-3.01 (m, 4H), 2.98-2.76 (m, 1H), 2.42-2.33 (m, 1H), 1.28-1.22 (m, 2H), 1.11-1.07 (in, 2H).

### Example 185: 1-[(3S,5R)-1-(But-2-ynoyl)-5-(methoxymethyl)pyrrolidin-3-yl]-3-[2-(1-cyclopropyl-4,6-difluoro-1,3-benzodiazol-5-yl)ethynyl]-5-(methylamino)pyrazole-4-carboxamide

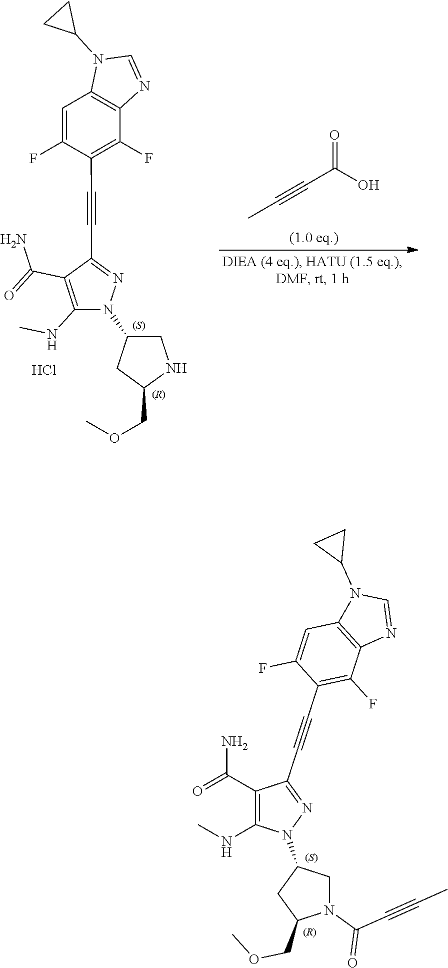

To a stirred solution of 3-((1-cyclopropyl-4,6-difluoro-1H-benzo[d]imidazol-5-yl)ethynyl)-1-((3S,5R)-5-(methoxymethyl)pyrrolidin-3-yl)-5-(methylamino)-1H-pyrazole-4-carboxamide hydrochloride (0.43 g, 0.93 mmol) in DMF (4.37 mL) were added 2-butynoic acid (78.25 mg, 0.93 mmol), HATU (0.53 g, 1.39 mmol) and DIEA (0.62 mL, 3.55 mmol) dropwise at 0° C. under nitrogen atmosphere. The reaction mixture was stirred for 1 h at room temperature. The resulting mixture was concentrated under reduced pressure. The residue was purified by silica gel column chromatography, eluted with DCM/MeOH (10/1) to afford the crude product which was further purified by Prep-HPLC with the following conditions: Column: XBridge Shield RP18 OBD Column, 30×150 mm, 5 μm; Mobile Phase A: water (10 mmol/L NH₄HCO₃), Mobile Phase B: ACN; Flow rate: 60 mL/min; Gradient: 28% B to 45% B in 8 min; 254/210 nm; RT1: 6.65 min. The fractions contained desired product were combined and concentrated to afford 1-[(3S,5R)-1-(but-2-ynoyl)-5-(methoxymethyl)pyrrolidin-3-yl]-3-[2-(1-cyclopropyl-4,6-difluoro-1,3-benzodiazol-5-yl)ethynyl]-5-(methylamino)pyrazole-4-carboxamide (0.14 g, 28%) as a white solid. MS ESI calculated for C₂₇H₂₇F₂N₇O₃[M+H]⁺, 536.21, found 536.15; ¹H NMR (400 MHz, CDCl₃) δ 7.97 (s, 1H), 7.19-7.16 (m, 1H), 7.08 (s, 1H), 5.49-5.32 (m, 2H), 4.55-4.45 (m, 1H), 4.20-3.99 (m, 1H), 3.93-3.86 (m, 1H), 3.77-3.56 (m, 1H), 3.43-3.40 (m, 2H), 3.37 (s, 3H), 3.06 (s, 3H), 2.96-2.74 (m, 1H), 2.39-2.30 (m, 1H), 2.00 (s, 3H), 1.27-1.22 (m, 2H), 1.11-1.07 (m, 2H).

### Example 186: 3-[2-(1-Cyclopropyl-4,6-difluoro-1,3-benzodiazol-5-yl)ethynyl]-5-(methylamino)-1-[(3S)-1-(prop-2-enoyl)pyrrolidin-3-yl]pyrazole-4-carboxamide

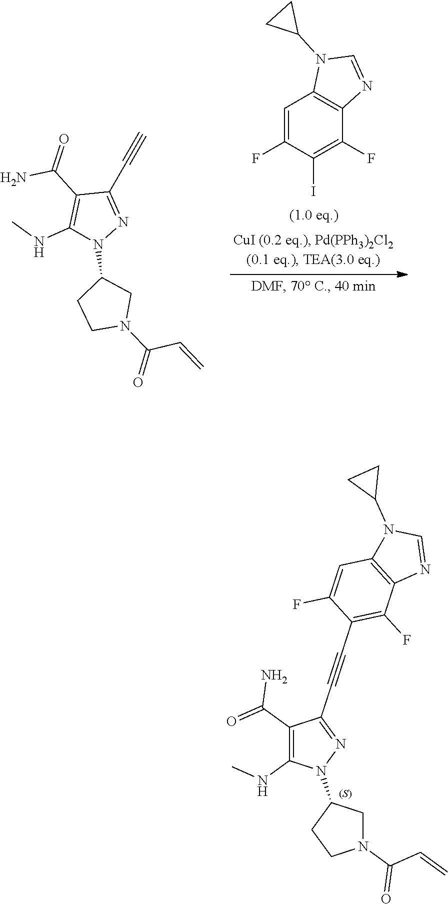

3-[2-(1-cyclopropyl-4,6-difluoro-1,3-benzodiazol-5-yl)ethynyl]-5-(methylamino)-1-[(3S)-1-(prop-2-enoyl)pyrrolidin-3-yl]pyrazole-4-carboxamide. MS ESI calculated for C24H₂₃F₂N₇O₂ [M+H]⁺, 480.49, found 480.35; ¹H NMR (400 MHz, DMSO-d₆) δ 8.41-8.39 (s, 1H), 7.58-7.50 (d, J=8.8 Hz, 2H), 6.80-6.54 (m, 3H), 6.17-6.10 (m, 1H), 5.77-5.65 (m, 1H), 5.24-5.09 (m, 1H), 4.09-3.80 (m, 2H), 3.78-3.69 (m, 1H), 3.68-3.49 (m, 2H), 2.97-2.90 (m, 3H), 2.42-2.40 (m, 1H), 2.32-2.30 (m, 1H), 1.17-1.04 (m, 4H).

### Example 187: 3-[2-(1-Cyclopropyl-4,6-difluoro-1,3-benzodiazol-5-yl)ethynyl]-1-[(3S,5R)-5-(hydroxymethyl)-1-(prop-2-enoyl)pyrrolidin-3-yl]-5-(methylamino)pyrazole-4-carboxamide

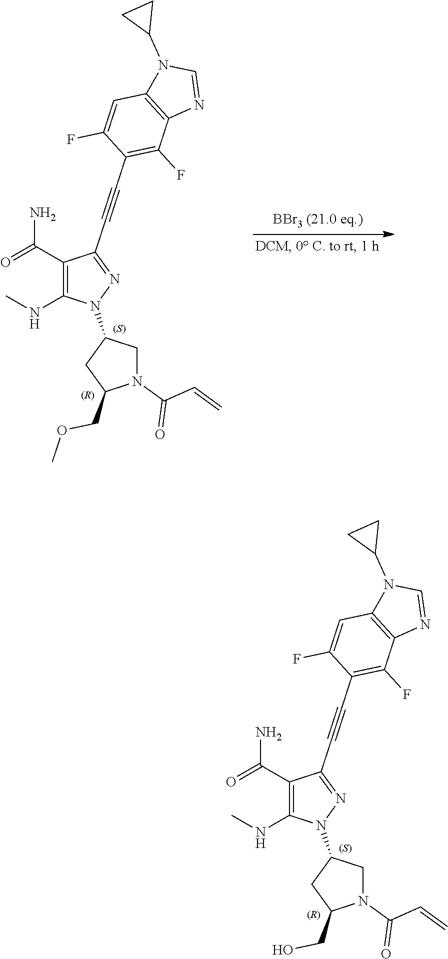

3-[2-(1-cyclopropyl-4,6-difluoro-1,3-benzodiazol-5-yl)ethynyl]-1-[(3S,5R)-5-(hydroxymethyl)-1-(prop-2-enoyl)pyrrolidin-3-yl]-5-(methylamino)pyrazole-4-carboxamide. MS ESI calculated for C₂₅H₂₅F₂N₇O₃[M+H]⁺, 510.20, found 510.15; ¹H NMR (400 MHz, DMSO-d₆) δ 8.42 (s, 1H), 7.59 (d, J=8.8 Hz, 2H), 6.80-6.54 (m, 3H), 6.17-6.05 (m, 1H), 5.68 (m, 1H), 5.31-5.24 (m, 1H), 5.02-4.93 (m, 1H), 4.34-4.22 (m, 1H), 4.09-4.05 (m, 1H), 3.94-3.73 (m, 1H), 3.68-3.54 (m, 1H), 3.59-3.47 (m, 2H), 3.29-2.96 (m, 3H), 2.68-2.53 (m, 1H), 2.48-2.25 (m, 1H), 1.22-1.01 (m, 4H).

### Example 188: 3-[2-(1-Cyclopropyl-6-fluoro-1,3-benzodiazol-5-yl)ethynyl]-5-(methylamino)-1-[1-(prop-2-ynoyl)pyrrolidin-3-yl]pyrazole-4-carboxamide

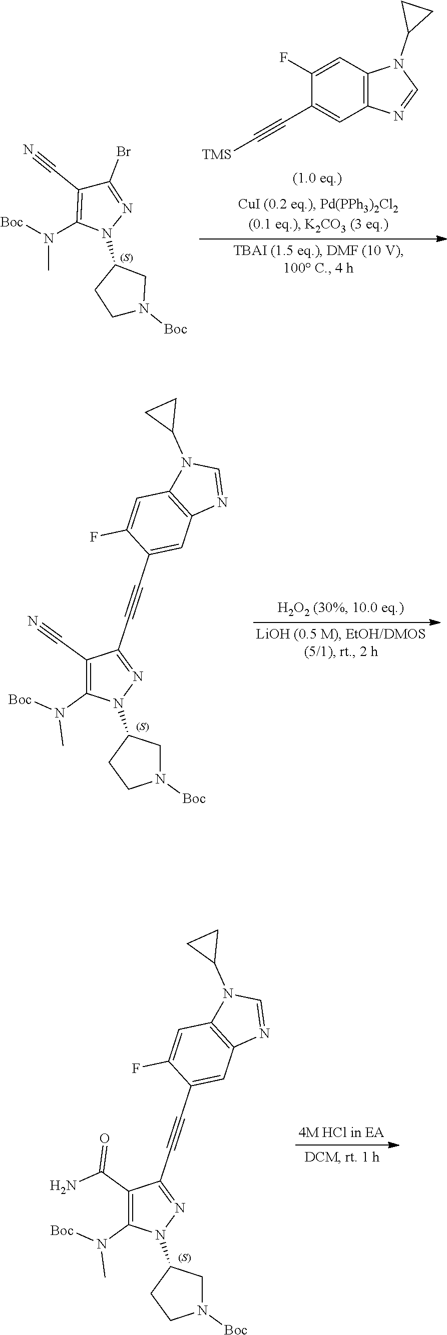

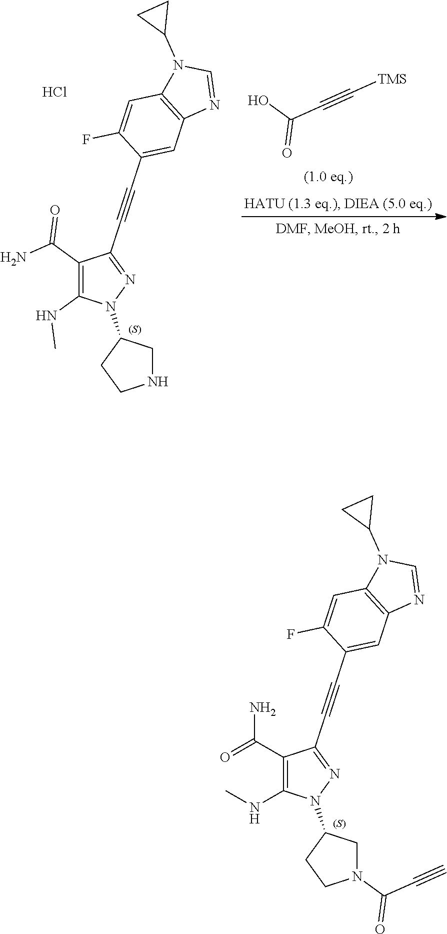

3-[2-(1-cyclopropyl-6-fluoro-1,3-benzodiazol-5-yl)ethynyl]-5-(methylamino)-1-[1-(prop-2-ynoyl)pyrrolidin-3-yl]pyrazole-4-carboxamide. MS ESI calculated for C₂₄H₂₂FN₇O₂ [M+H]⁺, 460.18, found 460.15; ¹H NMR (400 MHz, CDCl₃) δ 8.28 (d, J=12.8 Hz, 1H), 8.05 (s, 1H), 7.37 (d, J=8.6 Hz, 1H), 7.10 (s, 1H), 5.43 (s, 1H), 5.07 (s, 1H), 4.21-4.01 (m, 2H), 3.81 (d, J=90.7 Hz, 2H), 3.45 (s, 1H), 3.10 (d, J=15.7 Hz, 1H), 3.02 (d, J=11.0 Hz, 3H), 2.68 (s, 1H), 2.42 (s, 1H), 1.29-1.24 (m, 2H), 1.14 (s, 2H).

### Example 189: 5-Amino-3-[2-(1-cyclopropyl-4,6-difluoro-1,3-benzodiazol-5-yl)ethynyl]-1-[(3S,5R)-5-(methoxymethyl)-1-(prop-2-enoyl)pyrrolidin-3-yl]pyrazole-4-carboxamide

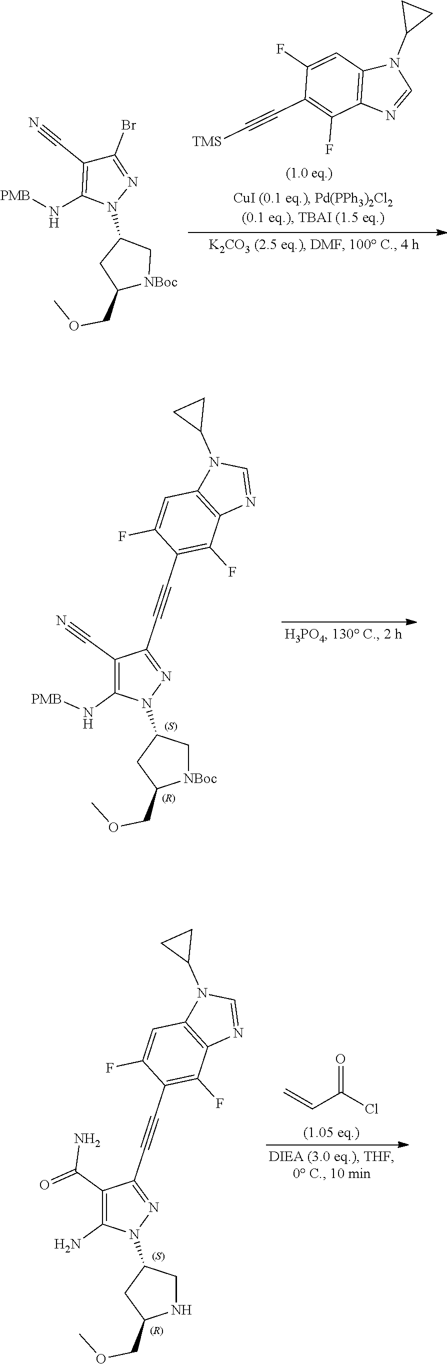

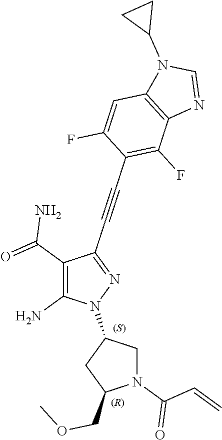

5-amino-3-[2-(1-cyclopropyl-4,6-difluoro-1,3-benzodiazol-5-yl)ethynyl]-1-[(3S,5R)-5-(methoxymethyl)-1-(prop-2-enoyl)pyrrolidin-3-yl]pyrazole-4-carboxamide. MS ESI calculated for C₂₅H₂₅F₂N₇O₃[M+H]⁺, 510.20, found 510.10; ¹H NMR (400 MHz, DMSO-d₆) δ 8.42 (s, 1H), 7.59 (d, J=8.8 Hz, 1H), 7.45 (s, 1H), 6.78-6.65 (m, 2H), 6.57-6.48 (m, 1H), 6.16-6.07 (m, 1H), 5.69-5.61 (m, 1H), 5.12 (q, J=8.6, 7.9 Hz, 1H), 4.56-4.34 (m, 1H), 4.00-3.65 (m, 2H), 3.57-3.48 (m, 2H), 3.42 (d, J=5.7 Hz, 1H), 3.37-3.32 (m, 3H), 2.51-2.42 (m, 1H), 2.31-2.22 (m, 1H), 1.17-1.03 (m, 4H).

### Example 190: 5-Amino-3-[2-(1-cyclopropyl-4,6-difluoro-1,3-benzodiazol-5-yl)ethynyl]-1-[(3S)-1-(prop-2-enoyl)pyrrolidin-3-yl]pyrazole-4-carboxamide

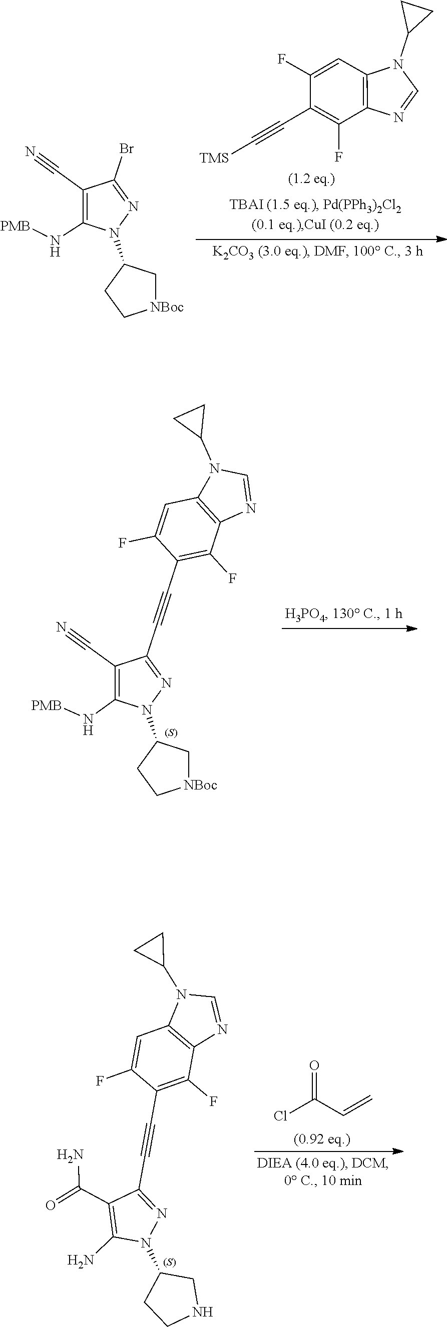

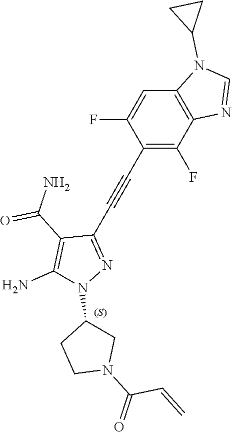

5-amino-3-[2-(1-cyclopropyl-4,6-difluoro-1,3-benzodiazol-5-yl)ethynyl]-1-[(S)-1-(prop-2-enoyl)pyrrolidin-3-yl]pyrazole-4-carboxamide. MS ESI calculated for C₂₃H₂₁F₂N₇O₂[M+H]⁺, 466.17, found 466.35; ¹H NMR (400 MHz, DMSO-d₆) δ 8.42 (s, 1H), 7.59 (d, J=8.9 Hz, 1H), 7.45 (s, 1H), 6.72-6.53 (m, 4H), 6.19-6.14 (m, 1H), 5.72-5.66 (m, 1H), 4.05-4.93 (m, 1H), 4.09-3.74 (m, 2H), 3.71-3.48 (m, 3H), 2.45-2.33 (m, 1H), 2.28-2.25 (m, 1H), 1.16-1.02 (i, 4H).

### Example 191: 3-[2-(6-Chloro-1-ethyl-1,3-benzodiazol-5-yl)ethynyl]-1-[(3S,5R)-5-(hydroxymethyl)-1-(prop-2-enoyl)pyrrolidin-3-yl]-5-(methylamino)pyrazole-4-carboxamide

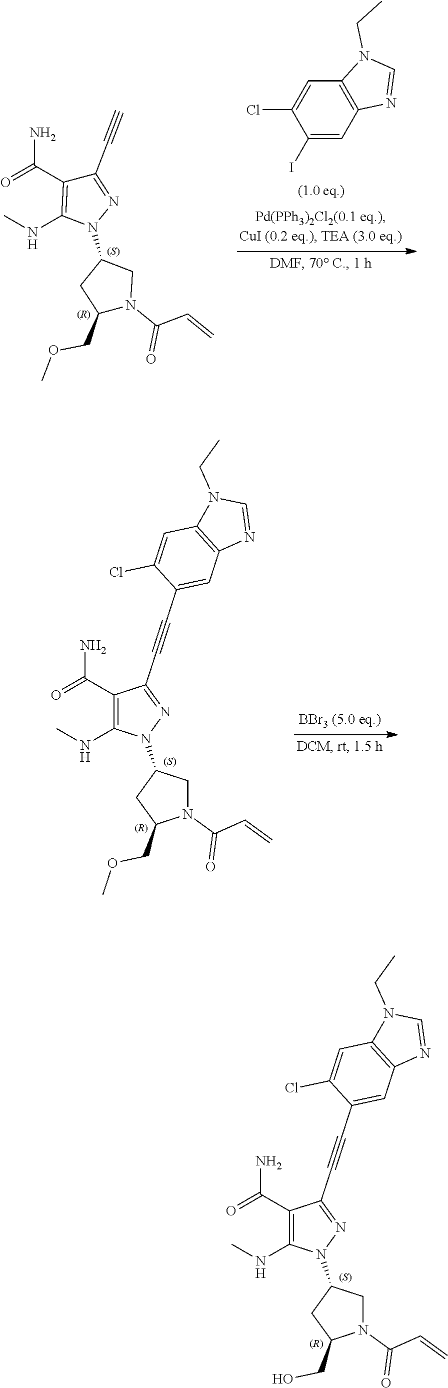

3-[2-(6-chloro-1-ethyl-1,3-benzodiazol-5-yl)ethynyl]-1-[(3S,5R)-5-(hydroxymethyl)-1-(prop-2-enoyl)pyrrolidin-3-yl]-5-(methylamino)pyrazole-4-carboxamide. MS ESI calculated for C₂₄H₂₆ClN₇O₃[M+H]⁺, 496.18, found 496.35; ¹H NMR (400 MHz, CDCl₃) δ 8.43 (s, 1H), 8.01 (d, J=4 Hz, 2H), 7.49 (s, 1H), 6.86 (s, 1H), 6.75-6.57 (m, 2H), 6.20-6.14 (m, 1H), 5.71-5.67 (m, 1H), 5.38-5.24 (m, 1H), 5.10 (t, J=12 Hz, 1H), 4.95 (t, J=12.6 Hz, 3H), 4.38 (d, J=4 Hz, 2H), 4.33-4.28 (m, 2H), 3.92-3.76 (m, 3H), 3.71-3.66 (m, 1H), 3.55-3.49 (m, 1H), 1.41 (t, J=12 Hz, 3H).

### Example 192: 3-[2-(6-Chloro-1-ethyl-1,3-benzodiazol-5-yl)ethynyl]-5-(methylamino)-1-[1-(prop-2-enoyl)azetidin-3-yl]pyrazole-4-carboxamide

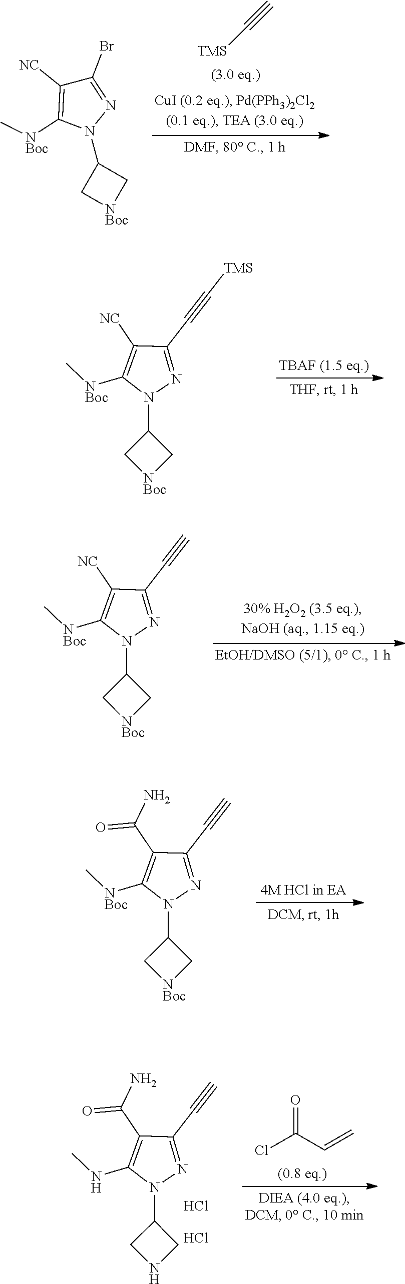

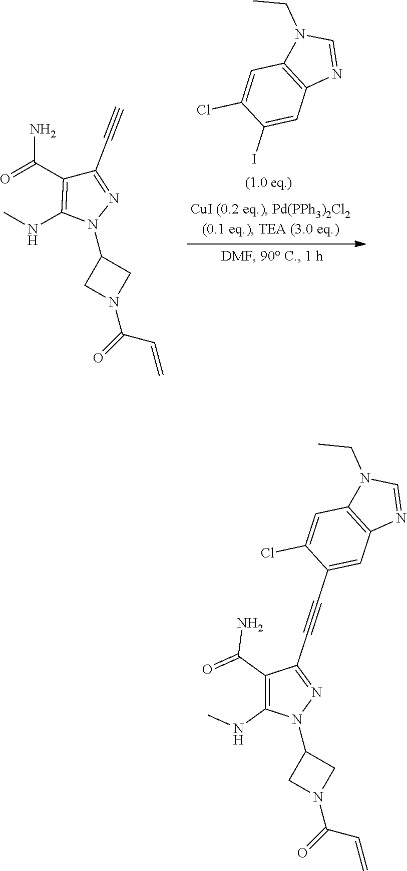 [SMILES: C#Cc1nn(C2CN(C(=O)C=C)C2)c(NC)c1C(N)=O ; CCn1cnc2cc(I)c(Cl)cc21 ; C=CC(=O)N1CC(n2nc(C#Cc3cc4ncn(CC)c4cc3Cl)c(C(N)=O)c2NC)C1]

### Step 1: Tert-butyl 3-(5-((tert-butoxycarbonyl)(methyl)amino)-4-cyano-3-((trimethylsilyl)ethynyl)-1H-pyrazol-1-yl)azetidine-1-carboxylate

To a stirred mixture of tert-butyl 3-{3-bromo-5-[(tert-butoxycarbonyl)(methyl)amino]-4-cyanopyrazol-1-yl}azetidine-1-carboxylate (2.80 g, 6.13 mmol), Pd(PPh₃)₂Cl₂ (0.43 g, 0.61 mmol) and CuI (0.23 g, 1.21 mmol) in DMF (28.00 mL) were added TEA (2.56 mL, 18.42 mmol) and trimethylsilylacetylene (1.81 g, 18.41 mmol) at room temperature. The reaction mixture was degassed with nitrogen for three times and stirred for 1 h at 80° C. The resulting mixture was concentrated under reduced pressure. The residue was purified by silica gel column chromatography, eluted with PE/EA (2/1). The fractions contained desired product were combined and concentrated to afford tert-butyl 3-(5-((tert-butoxycarbonyl)(methyl)amino)-4-cyano-3-((trimethylsilyl)ethynyl)-1H-pyrazol-1-yl)azetidine-1-carboxylate (2.90 g, 94%) as a light yellow solid. MS ESI calculated for C₂₃H₃₅N₅O₄Si [M+H−112]⁺, 362.25, found 362.00; ¹H NMR (400 MHz, CDCl₃) δ 4.85 (s, 1H), 4.47 (s, 1H), 4.28 (d, J=7.9 Hz, 3H), 3.23 (s, 3H), 1.46 (d, J=3.0 Hz, 18H), 0.29 (s, 9H).

### Step 2: Tert-butyl 3-(5-((tert-butoxycarbonyl)(methyl)amino)-4-cyano-3-ethynyl-1H-pyrazol-1-yl)azetidine-1-carboxylate

To a stirred solution of tert-butyl 3-(5-((tert-butoxycarbonyl)(methyl)amino)-4-cyano-3-((trimethylsilyl)ethynyl)-1H-pyrazol-1-yl)azetidine-1-carboxylate (2.84 g, 5.99 mmol) in THF (19.88 mL) was added TBAF (8.99 mL, 8.99 mmol) dropwise at room temperature under nitrogen atmosphere. The reaction mixture was stirred for 1 h at room temperature under nitrogen atmosphere. The residue was purified by silica gel column chromatography, eluted with PE/EA (2/1). The fractions contained desired product were combined and concentrated to afford tert-butyl 3-(5-((tert-butoxycarbonyl)(methyl)amino)-4-cyano-3-ethynyl-1H-pyrazol-1-yl)azetidine-1-carboxylate (2.00 g, 83%) as an off-white solid. MS ESI calculated for C₂₀H₂₇N₅O₄ [M+H−56]⁺, 346.21, found 346.10.

### Step 3: Tert-butyl 3-(5-((tert-butoxycarbonyl)(methyl)amino)-4-carbamoyl-3-ethynyl-1H-pyrazol-1-yl)azetidine-1-carboxylate

To a stirred solution of tert-butyl 3-(5-((tert-butoxycarbonyl)(methyl)amino)-4-cyano-3-ethynyl-1H-pyrazol-1-yl)azetidine-1-carboxylate (2 g, 4.98 mmol) in EtOH (20.00 mL) and DMSO (4.0 mL) were added H₂O₂ (30%) (1.35 mL, 17.43 mmol) and 0.5 M NaOH (11.46 mL, 5.73 mmol) dropwise at 0° C. under nitrogen atmosphere. Then the reaction mixture was stirred for 1 h at 0° C. The resulting mixture was extracted with EtOAc (3×100 mL). The combined organic layers was washed with brine (3×50 mL), dried over anhydrous Na₂SO₄. After filtration, the filtrate was concentrated under reduced pressure. The residue was purified by silica gel column chromatography, eluted with DCM/EA (2/1), the fractions contained desired product were combined and concentrated to afford tert-butyl 3-(5-((tert-butoxycarbonyl)(methyl)amino)-4-carbamoyl-3-ethynyl-1H-pyrazol-1-yl)azetidine-1-carboxylate (2.00 g, 95%) as an off-white solid. MS ESI calculated for C₂₀H₂₉N₅O₅ [M+H]⁺, 420.22, found 420.20.

### Step 4: 1-(Azetidin-3-yl)-3-ethynyl-5-(methylamino)pyrazole-4-carboxamide dihydrochloride

To a stirred solution of tert-butyl 3-(5-((tert-butoxycarbonyl)(methyl)amino)-4-carbamoyl-3-ethynyl-1H-pyrazol-1-yl)azetidine-1-carboxylate (2.10 g, 4.51 mmol) in DCM (10.00 mL) was added 4 M HCl in EA (20.00 mL) dropwise at 0° C. under nitrogen atmosphere. The reaction mixture was stirred for 1 h at room temperature under nitrogen atmosphere. The precipitated solids were collected by filtration and washed with DCM (3×50 mL). The crude product 1-(azetidin-3-yl)-3-ethynyl-5-(methylamino)pyrazole-4-carboxamide dihydrochloride (0.77 g, crude) was used in the next step directly without further purification. MS ESI calculated for C₁₀H₁₅Cl₂N₅O [M+H−2HCl]⁺, 220.07, found 220.25.

### Step 5: 3-Ethynyl-5-(methylamino)-1-[1-(prop-2-enoyl)azetidin-3-yl]pyrazole-4-carboxamide

To a stirred solution of 1-(azetidin-3-yl)-3-ethynyl-5-(methylamino)pyrazole-4-carboxamide dihydrochloride (0.77 g, 1.85 mmol) in DCM (10.00 mL) were added acryloyl chloride (5.90 mL, 1.48 mmol) and DIEA (1.29 mL, 7.38 mmol) dropwise at 0° C. under nitrogen atmosphere. The reaction mixture was stirred for 10 min at 0° C. under nitrogen atmosphere. The resulting mixture was diluted with water (30.00 mL) and extracted with EA (3×50 mL). The combined organic layers was washed with brine (3×20 mL), dried over anhydrous Na₂SO₄. After filtration, the filtrate was concentrated under reduced pressure. The residue was purified by silica gel column chromatography, eluted with CH₂Cl₂/MeOH (10/1). The fractions contained desired product were combined and concentrated to afford 3-ethynyl-5-(methylamino)-1-[1-(prop-2-enoyl)azetidin-3-yl]pyrazole-4-carboxamide (0.30 g, 60%) as a white solid. MS ESI calculated for C₁₃H₁₅N₅O₂ [M+H]⁺, 274.12, found 274.10; ¹H NMR (400 MHz, Chloroform-d) δ 6.80 (s, 2H), 6.45-6.29 (m, 1H), 6.34-6.17 (m, 2H), 6.14-6.02 (m, 1H), 4.56 (s, 3H), 4.03-3.88 (m, 1H), 3.85-3.65 (m, 1H), 3.50 (d, J=2.7 Hz, 1H), 2.95 (s, 3H).

### Step 6: 3-[2-(6-Chloro-1-ethyl-1,3-benzodiazol-5-yl)ethynyl]-5-(methylamino)-1-[1-(prop-2-enoyl)azetidin-3-yl]pyrazole-4-carboxamide

To a stirred mixture of 3-ethynyl-5-(methylamino)-1-[1-(prop-2-enoyl)azetidin-3-yl]pyrazole-4-carboxamide (80.00 mg, 0.29 mmol), 6-chloro-1-ethyl-5-iodo-1,3-benzodiazole (89.73 mg, 0.29 mmol), CuI (11.15 mg, 0.06 mmol) and Pd(PPh₃)₂Cl₂ (20.55 mg, 0.03 mmol) in DMF (2.00 mL) was added TEA (0.12 mL, 0.88 mmol). The reaction mixture was degassed with nitrogen for three times and stirred for 1 h at 90° C. The resulting mixture was diluted with water (20.00 mL). The resulting mixture was extracted with EA (3×50 mL). The combined organic layers were washed with brine (3×30 mL), dried over anhydrous Na₂SO₄. After filtration, the filtrate was concentrated under reduced pressure. The residue was purified by silica gel column chromatography, eluted with CH₂Cl₂/MeOH (10/1) to afford the crude product which was further purified by Prep-HPLC with the following conditions: Column: Xselect CSH OBD Column 30×150 mm 5 μm; Mobile Phase A: water (0.1% FA), Mobile Phase B: ACN; Flow rate: 60 mL/min; Gradient: 20% B to 30% B in 8 min; Wave Length: 220 nm; RT1: 6.62 min. The fractions contained desired product were combined and concentrated to afford 3-[2-(6-chloro-1-ethyl-1,3-benzodiazol-5-yl)ethynyl]-5-(methylamino)-1-[1-(prop-2-enoyl)azetidin-3-yl]pyrazole-4-carboxamide (28.10 mg, 21%) as a white solid. MS ESI calculated for C₂₂H₂₂ClN₇O₂ [M+H]⁺, 452.15, found 452.35; ¹H NMR (400 MHz, DMSO-d₆) δ 8.43 (s, 1H), 8.06 (s, 1H), 8.01 (s, 1H), 7.52 (s, 1H), 6.87 (s, 1H), 6.83-6.67 (m, 1H), 6.45-6.29 (m, 1H), 6.24-6.09 (m, 1H), 5.80-5.68 (m, 1H), 5.51-5.39 (m, 1H), 4.73-4.52 (m, 2H), 4.47-4.22 (m, 4H), 2.91 (d, J=5.7 Hz, 3H), 1.41 (t, J=7.2 Hz, 3H).

### Example 193: 3-[2-(1-Cyclopropyl-4,6-difluoro-1,3-benzodiazol-5-yl)ethynyl]-5-(methylamino)-1-[1-(prop-2-enoyl)azetidin-3-yl]pyrazole-4-carboxamide

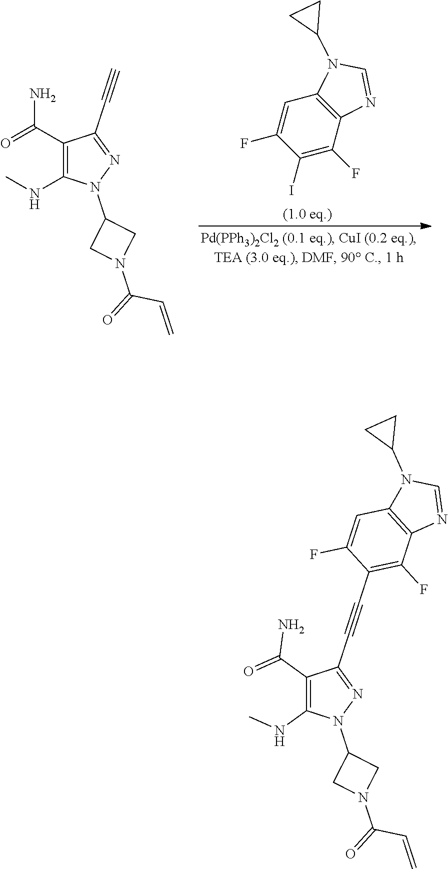 [SMILES: C#Cc1nn(C2CN(C(=O)C=C)C2)c(NC)c1C(N)=O ; Fc1cc2c(ncn2C2CC2)c(F)c1I ; C=CC(=O)N1CC(n2nc(C#Cc3c(F)cc4c(ncn4C4CC4)c3F)c(C(N)=O)c2NC)C1]

3-[2-(1-cyclopropyl-4,6-difluoro-1,3-benzodiazol-5-yl)ethynyl]-5-(methylamino)-1-[1-(prop-2-enoyl)azetidin-3-yl]pyrazole-4-carboxamide. MS ESI calculated for C₂₃H₂₁F₂N₇O₂[M+H]⁺, 466.17, found 466.40; ¹H NMR (400 MHz, DMSO-d₆) δ 8.43 (s, 1H), 7.60 (d, J=8.7 Hz, 2H), 6.76 (q, J=6.8, 6.2 Hz, 2H), 6.46-6.23 (m, 1H), 6.24-6.07 (m, 1H), 5.79-5.65 (m, 1H), 5.51-5.34 (m, 1H), 4.72-4.56 (m, 2H), 4.39 (t, J=9.2 Hz, 1H), 4.37-4.23 (m, 1H), 3.61-3.46 (m, 1H), 2.91 (d, J=5.7 Hz, 3H), 1.17-1.03 (m, 4H).

### Example 194: 5-Amino-3-[2-(6-chloro-1-ethyl-1,3-benzodiazol-5-yl)ethynyl]-1-[(3S,5R)-5-(hydroxymethyl)-1-(prop-2-enoyl)pyrrolidin-3-yl]pyrazole-4-carboxamide

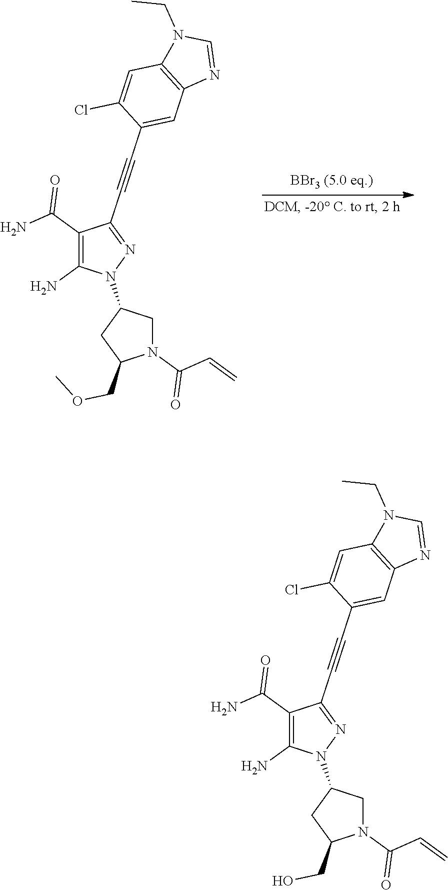 [SMILES: C=CC(=O)N1C[C@@H](n2nc(C#Cc3cc4ncn(CC)c4cc3Cl)c(C(N)=O)c2N)C[C@@H]1COC ; C=CC(=O)N1C[C@@H](n2nc(C#Cc3cc4ncn(CC)c4cc3Cl)c(C(N)=O)c2N)C[C@@H]1CO]

To a solution of 5-amino-3-[2-(6-chloro-1-ethyl-1,3-benzodiazol-5-yl)ethynyl]-1-[(3S,5R)-5-(methoxymethyl)-1-(prop-2-enoyl)pyrrolidin-3-yl]pyrazole-4-carboxamide (0.44 g, 0.89 mmol) in DCM (22.00 mL) was added BBr₃ (1.11 g, 4.44 mmol) dropwise at −20° C. The reaction mixture was allowed to warm to room temperature and stirred for 2 h. The resulting mixture was quenched with NaHCO₃ (aq.). and concentrated under reduced pressure. The residue product was purified by reverse phase flash with the following conditions: C18 silica gel; mobile phase: ACN in water (10 mmol/L NH₄HCO₃), 5% to 70% gradient in 30 min; detector: UV 254 nm. The fractions contained desired product were combined and concentrated to afford 5-amino-3-[2-(6-chloro-1-ethyl-1,3-benzodiazol-5-yl)ethynyl]-1-[(3S,5R)-5-(hydroxymethyl)-1-(prop-2-enoyl)pyrrolidin-3-yl]pyrazole-4-carboxamide (0.18 g, 42%) as an off-white solid. MS ESI calculated for C₂₃H₂₄ClN₇O₃[M+H]⁺, 482.17, found 482.30; ¹H NMR (400 MHz, DMSO-d₆) δ 8.43 (s, 1H), 8.01 (d, J=6.2 Hz, 2H), 7.35 (s, 1H), 6.96-6.46 (m, 4H), 6.22-6.12 (m, 1H), 5.69-5.67 (m, 1H), 5.14-5.05 (m, 1H), 4.89-4.88 (m, 1H), 4.33-4.27 (m, 3H), 4.01-3.66 (m, 2H), 3.58-3.55 (m, 1H), 3.55-3.45 (m, 1H), 2.48-2.11 (m, 2H), 1.41 (t, J=7.2 Hz, 3H).

### Example 195: 3-[2-(6-Chloro-1-cyclopropyl-1,3-benzodiazol-5-yl)ethynyl]-1-[(3S,5R)-5-(hydroxymethyl)-1-(prop-2-enoyl)pyrrolidin-3-yl]-5-(methylamino)pyrazole-4-carboxamide

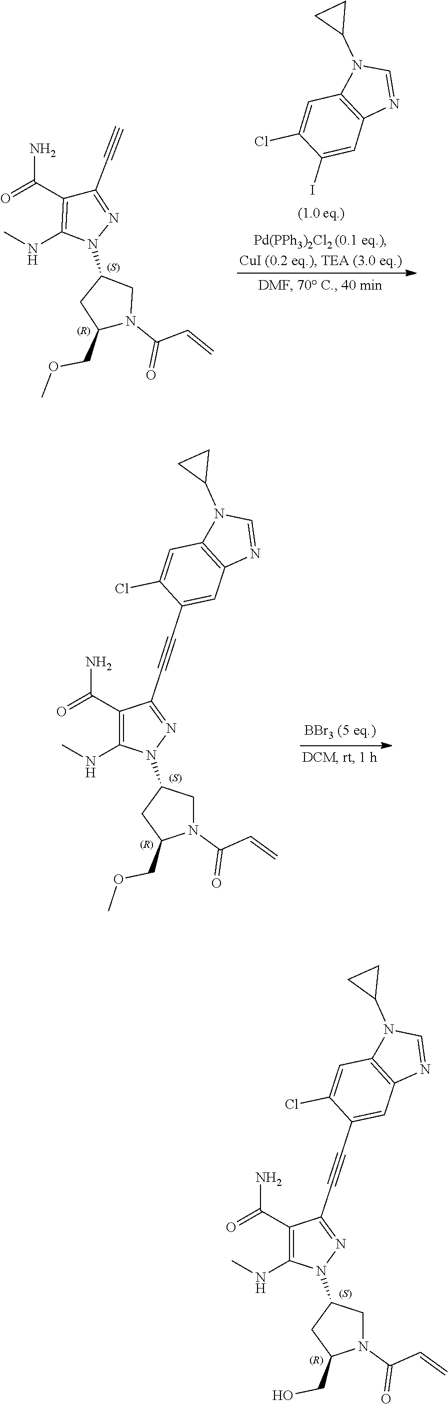

3-[2-(6-chloro-1-cyclopropyl-1,3-benzodiazol-5-yl)ethynyl]-1-[(3S,5R)-5-(hydroxymethyl)-1-(prop-2-enoyl)pyrrolidin-3-yl]-5-(methylamino)pyrazole-4-carboxamide. MS ESI calculated for C₂₅H₂₆ClN₇O₃[M+H]⁺, 508.18, found 508.10; ¹H NMR (400 MHz, CDCl₃) δ 8.08 (d, J=13.3 Hz, 2H), 7.68 (s, 1H), 7.20 (s, 1H), 6.57-6.40 (m, 2H), 5.78-5.66 (m, 1H), 5.52-5.21 (m, 2H), 4.55 (d, J=73.4 Hz, 1H), 4.23-4.12 (m, 1H), 4.10-3.94 (m, 2H), 3.73-3.62 (m, 1H), 3.41-3.28 (m, 1H), 3.03 (s, 3H), 2.81-2.66 (m, 1H), 2.21-2.13 (m, 1H), 1.29-1.18 (m, 2H), 1.18-1.06 (m, 2H).

### Example 196: 5-Amino-3-[2-(6-chloro-1-ethyl-1,3-benzodiazol-5-yl)ethynyl]-1-[(3S,5R)-5-(methoxymethyl)-1-(prop-2-enoyl)pyrrolidin-3-yl]pyrazole-4-carboxamide

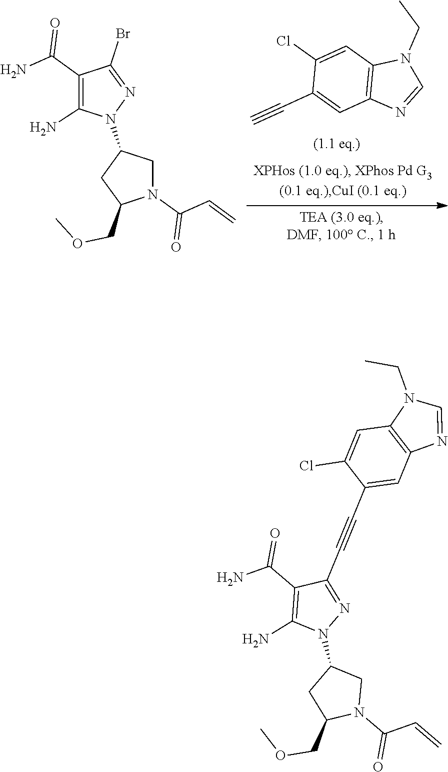 [SMILES: C=CC(=O)N1C[C@@H](n2nc(Br)c(C(N)=O)c2N)C[C@@H]1COC ; C#Cc1cc2ncn(CC)c2cc1Cl ; C=CC(=O)N1C[C@@H](n2nc(C#Cc3cc4ncn(CC)c4cc3Cl)c(C(N)=O)c2N)C[C@@H]1COC]

5-amino-3-[2-(6-chloro-1-ethyl-1,3-benzodiazol-5-yl)ethynyl]-1-[(3S,5R)-5-(methoxymethyl)-1-(prop-2-enoyl)pyrrolidin-3-yl]pyrazole-4-carboxamide. MS ESI calculated for C₂₄H₂₆ClN₇O₃[M+H]⁺, 496.19, found 496.15; ¹H NMR (400 MHz, CDCl₃) δ 8.10 (d, J=4.6 Hz, 1H), 7.98 (s, 1H), 7.51 (s, 1H), 7.16 (s, 1H), 6.46-6.42 (m, 2H), 5.73-5.70 (m, 1H), 5.64 (s, 2H), 5.46 (s, 1H), 5.08-5.03 (m, 1H), 4.59 (d, J=9.1 Hz, 1H), 4.25-4.20 (m, 2H), 4.09-3.83 (m, 3H), 3.51-3.46 (m, 1H), 3.39 (s, 3H), 2.69 (m, 1H), 2.36-2.31 (m, 1H), 1.58 (t, J=7.3 Hz, 3H).

### Example 197: 3-[2-(6-Chloro-1-cyclopropyl-1,3-benzodiazol-5-yl)ethynyl]-5-(methylamino)-1-[1-(prop-2-enoyl)azetidin-3-yl]pyrazole-4-carboxamide

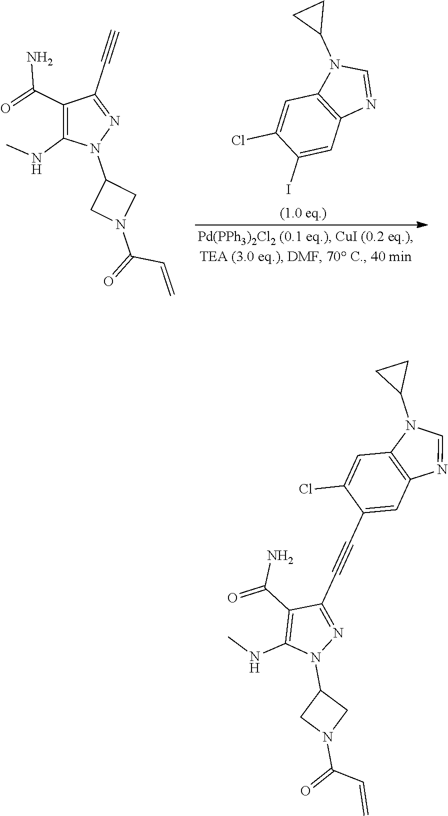 [SMILES: C#Cc1nn(C2CN(C(=O)C=C)C2)c(NC)c1C(N)=O ; Clc1cc2c(cc1I)ncn2C1CC1 ; C=CC(=O)N1CC(n2nc(C#Cc3cc4ncn(C5CC5)c4cc3Cl)c(C(N)=O)c2NC)C1]

3-[2-(6-chloro-1-cyclopropyl-1,3-benzodiazol-5-yl)ethynyl]-5-(methylamino)-1-[1-(prop-2-enoyl)azetidin-3-yl]pyrazole-4-carboxamide. MS ESI calculated for C₂₃H₂₂ClN₇O₂ [M+H]⁺, 464.15, found 464.35; ¹H NMR (400 MHz, CDCl₃) δ 8.23 (s, 1H), 7.88 (s, 1H), 7.23-7.09 (m, 1H), 6.72 (s, 1H), 6.48-6.27 (m, 1H), 6.30-6.13 (m, 1H), 5.75-5.72 (m, 1H), 5.42 (s, 1H), 5.33-5.20 (m, 1H), 4.99 (d, J=8.1 Hz, 1H), 4.63-4.52 (m, 3H), 3.41 (s, 1H), 2.97 (s, 3H), 1.26 (t, J=7.4 Hz, 2H), 1.11 (s, 2H).

### Example 198: 3-[2-(1-Cyclopropyl-6-fluoro-1,3-benzodiazol-5-yl)ethynyl]-1-[(3S,5R)-5-(hydroxymethyl)-1-(prop-2-ynoyl)pyrrolidin-3-yl]-5-(methylamino)pyrazole-4-carboxamide

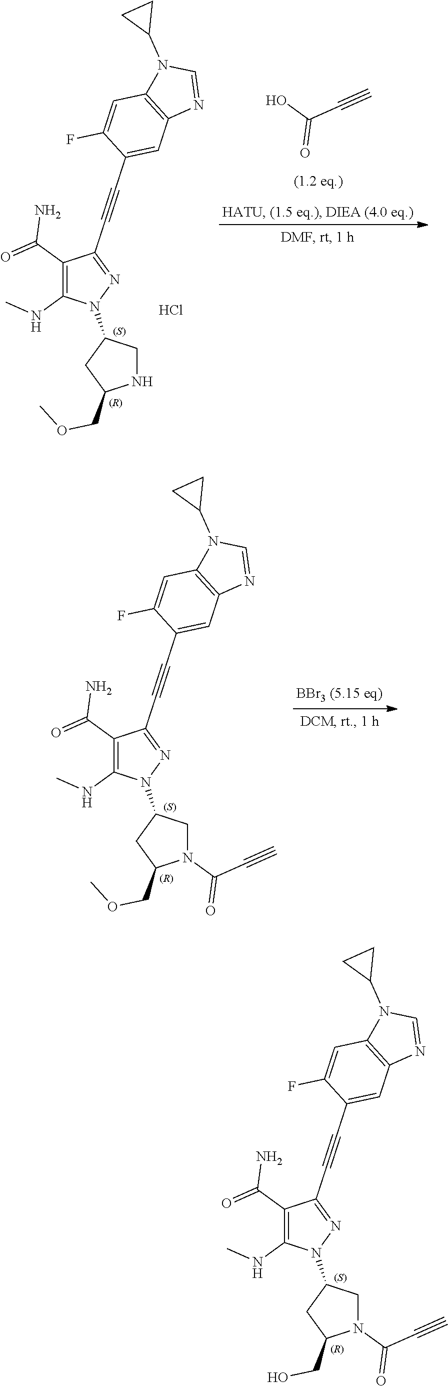

3-[2-(1-cyclopropyl-6-fluoro-1,3-benzodiazol-5-yl)ethynyl]-1-[(3S,5R)-5-(hydroxymethyl)-1-(prop-2-ynoyl)pyrrolidin-3-yl]-5-(methylamino)pyrazole-4-carboxamide (19.30 mg, 19%) as an off-white solid. MS ESI calculated for C₂₅H₂₄FN₇O₃[M+H]⁺, 490.20, found 490.20; ¹H NMR (400 MHz, DMSO-d₆) δ 8.36 (s, 1H), 7.96 (d, J=2.6 Hz, 1H), 7.63 (s, 1H), 7.47 (s, 1H), 6.72-6.65 (m, 2H), 5.16-5.13 (m, 1H), 4.98-4.96 (m, 1H), 4.39 (s, 1H), 4.24 (s, 1H), 3.89-3.82 (m, 1H), 3.76-3.70 (m, 2H), 3.52-3.49 (m, 2H), 2.95 (d, J=1.5 Hz, 3H), 2.63-2.59 (m, 1H), 2.42-2.33 (m, 1H), 1.10-1.06 (in, 4H).

### Example 199: 5-Amino-3-[2-(6-chloro-1-cyclopropyl-1,3-benzodiazol-5-yl)ethynyl]-1-[(3S,5R)-5-(hydroxymethyl)-1-(prop-2-enoyl)pyrrolidin-3-yl]pyrazole-4-carboxamide

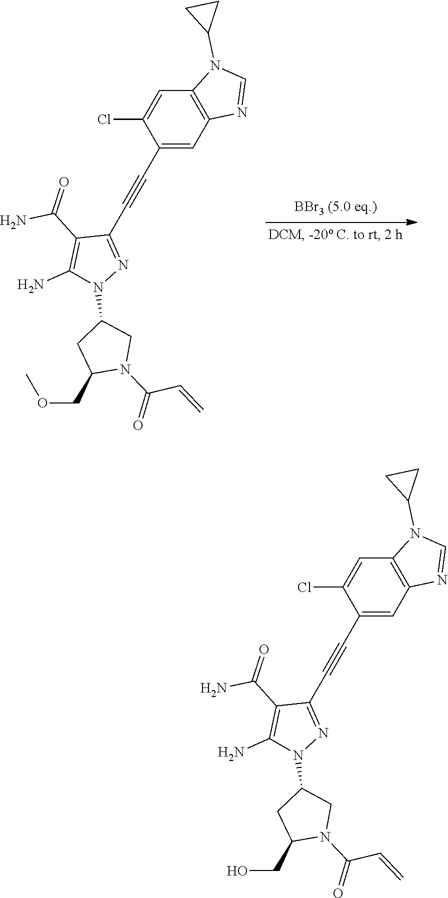 [SMILES: C=CC(=O)N1C[C@@H](n2nc(C#Cc3cc4ncn(C5CC5)c4cc3Cl)c(C(N)=O)c2N)C[C@@H]1COC ; C=CC(=O)N1C[C@@H](n2nc(C#Cc3cc4ncn(C5CC5)c4cc3Cl)c(C(N)=O)c2N)C[C@@H]1CO]

5-amino-3-[2-(6-chloro-1-cyclopropyl-1,3-benzodiazol-5-yl)ethynyl]-1-[(3S,5R)-5-(hydroxymethyl)-1-(prop-2-enoyl)pyrrolidin-3-yl]pyrazole-4-carboxamide. MS ESI calculated for C₂₄H₂₄ClN₇O₃[M+H]⁺, 494.16, found 494.15; ¹H NMR (400 MHz, DMSO-d₆) δ 8.39 (s, 1H), 8.02 (s, 1H), 7.88 (s, 1H), 7.35 (s, 1H), 6.84-6.45 (m, 4H), 6.18-6.14 (m, 1H), 5.69-5.67 (m, 1H), 5.20-4.85 (m, 2H), 4.38-4.28 (m, 1H), 4.03-3.66 (m, 2H), 3.56-3.51 (m, 2H), 3.48 (d, J=5.2 Hz, 1H), 2.44-2.27 (m, 2H), 1.17-1.02 (in, 4H).

### Example 200: 5-Amino-3-[2-(6-chloro-1-cyclopropyl-1,3-benzodiazol-5-yl)ethynyl]-1-[(3S,5R)-5-(methoxymethyl)-1-(prop-2-enoyl)pyrrolidin-3-yl]pyrazole-4-carboxamide

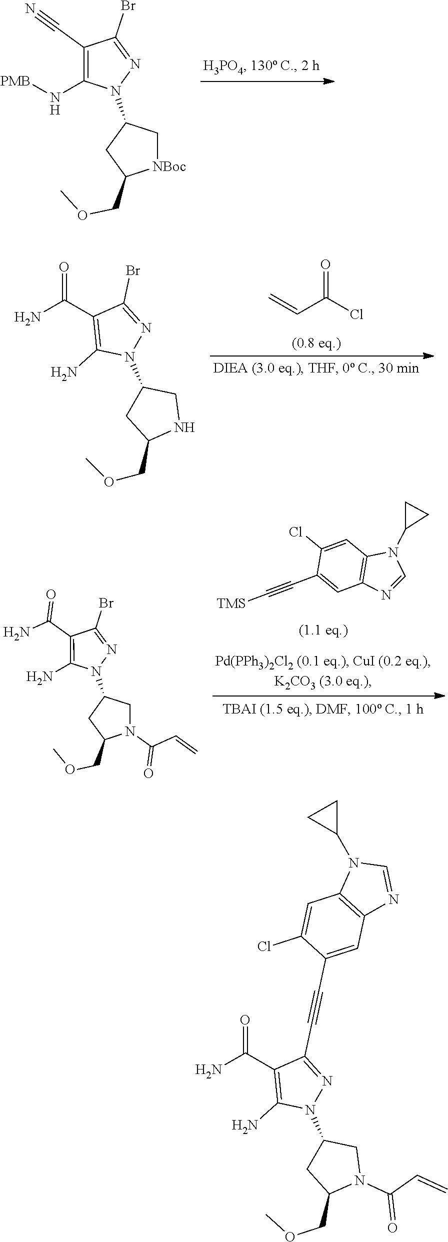

5-amino-3-[2-(6-chloro-1-cyclopropyl-1,3-benzodiazol-5-yl)ethynyl]-1-[(3S,5R)-5-(methoxymethyl)-1-(prop-2-enoyl)pyrrolidin-3-yl]pyrazole-4-carboxamide. MS ESI calculated for C₂₅H₂₆ClN₇O₃[M+H]+, 508.19, found 508.35; ¹H NMR (400 MHz, CDCl₃) δ 8.05 (d, J=27.4 Hz, 2H), 7.67 (s, 1H), 7.16 (s, 1H), 6.51-6.36 (m, 2H), 5.75-5.72 (m, 1H), 5.60 (s, 2H), 5.44 (s, 1H), 5.09-5.05 (m, 1H), 4.60 (d, J=8.9 Hz, 1H), 4.06 (d, J=8.2 Hz, 2H), 3.99-3.86 (m, 1H), 3.54-3.50 (m, 1H), 3.39 (s, 4H), 2.72-2.65 (m, 1H), 2.47-2.29 (m, 1H), 1.25-1.20 (m, 2H), 1.10-1.06 (m, 2H).

### Example 201: 5-Amino-3-[2-(1-cyclopropyl-4,6-difluoro-1,3-benzodiazol-5-yl)ethynyl]-1-[(3S,5R)-5-(hydroxymethyl)-1-(prop-2-enoyl)pyrrolidin-3-yl]pyrazole-4-carboxamide

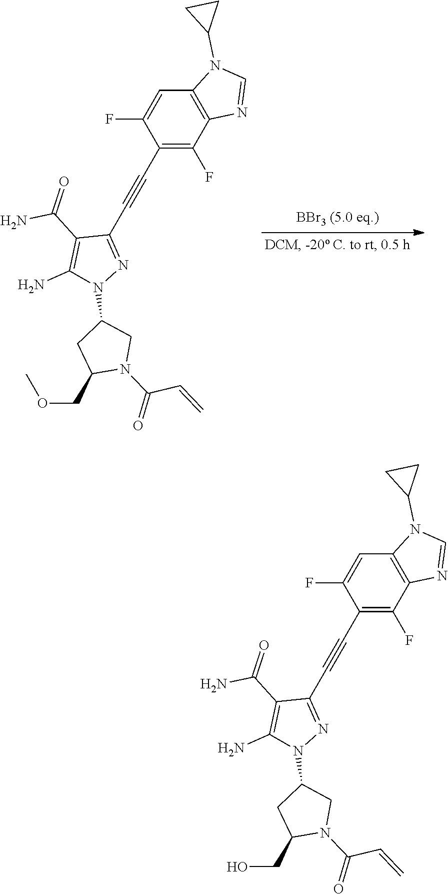 [SMILES: C=CC(=O)N1C[C@@H](n2nc(C#Cc3c(F)cc4c(ncn4C4CC4)c3F)c(C(N)=O)c2N)C[C@@H]1COC ; C=CC(=O)N1C[C@@H](n2nc(C#Cc3c(F)cc4c(ncn4C4CC4)c3F)c(C(N)=O)c2N)C[C@@H]1CO]

5-amino-3-[2-(1-cyclopropyl-4,6-difluoro-1,3-benzodiazol-5-yl)ethynyl]-1-[(3S,5R)-5-(hydroxymethyl)-1-(prop-2-enoyl)pyrrolidin-3-yl]pyrazole-4-carboxamide. MS ESI calculated for C₂₄H₂₃F₂N₇O₃[M+H]+, 496.19, found 496.15; ¹H NMR (300 MHz, DMSO-d₆) δ 8.43 (s, 1H), 7.61 (d, J=8.8 Hz, 1H), 7.47 (s, 1H), 6.70 (s, 3H), 6.59-6.53 (m, 1H), 6.17 (d, J=16.6 Hz, 1H), 5.69 (d, J=10.4 Hz, 1H), 5.13 (s, 2H), 4.32 (d, J=15.6 Hz, 1H), 3.98-3.71 (m, 2H), 3.59-3.40 (m, 3H), 2.43 (d, J=57.7 Hz, 2H), 1.11 (t, J=7.7 Hz, 4H).

### Example 202: 5-Amino-3-[2-(1-cyclopropyl-6-fluoro-1,3-benzodiazol-5-yl)ethynyl]-1-[1-(prop-2-enoyl)azetidin-3-yl]pyrazole-4-carboxamide

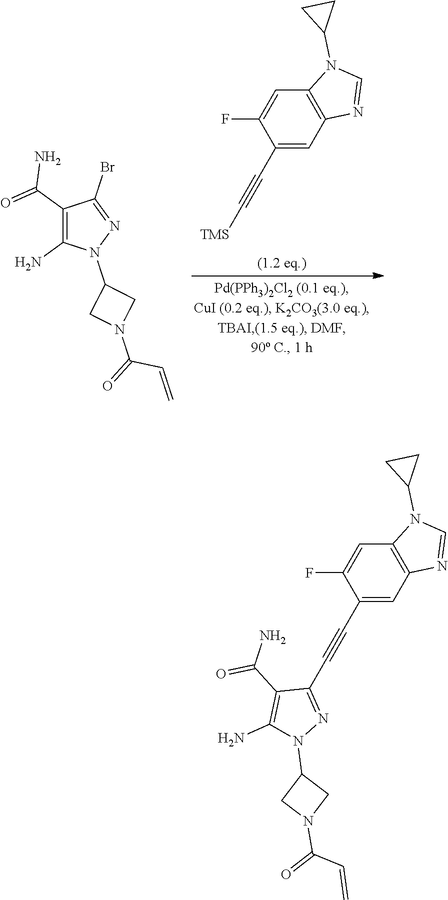 [SMILES: C=CC(=O)N1CC(n2nc(Br)c(C(N)=O)c2N)C1 ; C[Si](C)(C)C#Cc1cc2ncn(C3CC3)c2cc1F ; C=CC(=O)N1CC(n2nc(C#Cc3cc4ncn(C5CC5)c4cc3F)c(C(N)=O)c2N)C1]

5-amino-3-[2-(1-cyclopropyl-6-fluoro-1,3-benzodiazol-5-yl)ethynyl]-1-[1-(prop-2-enoyl)azetidin-3-yl]pyrazole-4-carboxamide. MS ESI calculated for C₂₂H₂₀FN₇O₂[M+H]⁺, 434.17, found 434.14; ¹H NMR (300 MHz, DMSO-d₆) δ 8.38 (s, 1H), 8.01 (d, J=6.4 Hz, 1H), 7.66 (d, J=9.5 Hz, 1H), 7.40 (s, 1H), 6.66 (s, 3H), 6.43-6.39 (m, 1H), 6.23-6.11 (m, 1H), 5.79-5.67 (m, 1H), 5.29 (s, 1H), 4.65 (t, J=8.6 Hz, 1H), 4.52 (d, J=7.7 Hz, 1H), 4.35 (t, J=9.3 Hz, 1H), 4.21 (t, J=7.5 Hz, 1H), 3.54 (d, J=6.3 Hz, 1H), 1.16-1.00 (m, 4H).

### Example 203: 3-((1-Cyclopropyl-6-fluoro-1H-benzo[d]imidazol-5-yl)ethynyl)-1-((3S,5R)-1-(4-hydroxy-4-methylpent-2-ynoyl)-5-(methoxymethyl)pyrrolidin-3-yl)-5-(methylamino)-1H-pyrazole-4-carboxamide

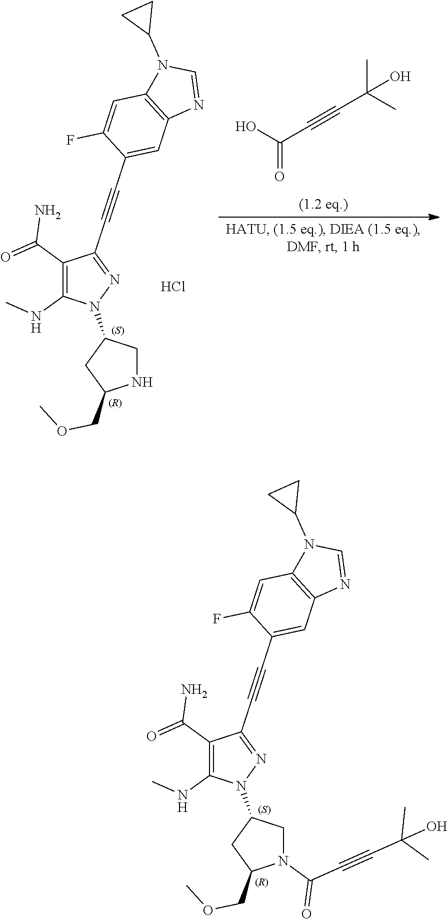

3-((1-cyclopropyl-6-fluoro-1H-benzo[d]imidazol-5-yl)ethynyl)-1-((3S,5R)-1-(4-hydroxy-4-methylpent-2-ynoyl)-5-(methoxymethyl)pyrrolidin-3-yl)-5-(methylamino)-1H-pyrazole-4-carboxamide (44.40 mg, 30%) as a white solid. MS ESI calculated for C₂₉H₃₂FN₇O₄ [M+H]⁺, 562.25, found 562.45; ¹H NMR (400 MHz, CDCl₃) δ 8.02 (d, J=50.8 Hz, 2H), 7.33 (s, 1H), 7.11 (s, 1H), 6.79 (s, 1H), 5.63-5.24 (m, 1H), 4.62-4.39 (m, 2H), 4.22-3.98 (m, 2H), 3.88 (d, J=9.6 Hz, 1H), 3.70-3.50 (m, 1H), 3.40 (d, J=13.5 Hz, 5H), 3.03 (d, J=10.3 Hz, 3H), 2.94-2.70 (m, 1H), 2.40-2.25 (m, 1H), 1.60 (d, J=12.3 Hz, 6H), 1.35-0.97 (m, 4H).

### Example 204: 3-[2-(1-Cyclopropyl-6-fluoro-1,3-benzodiazol-5-yl)ethynyl]-1-{1-[(2E)-4-(dimethylamino)but-2-enoyl]azetidin-3-yl}-5-(methylamino)pyrazole-4-carboxamide

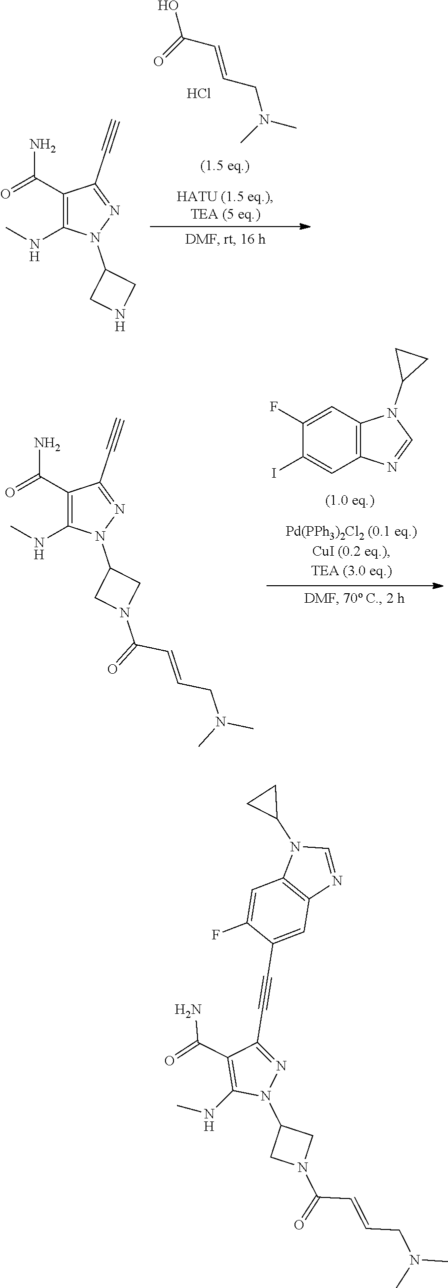

3-[2-(1-cyclopropyl-6-fluoro-1,3-benzodiazol-5-yl)ethynyl]-1-{1-[(2E)-4-(dimethylamino)but-2-enoyl]azetidin-3-yl}-5-(methylamino)pyrazole-4-carboxamide. MS ESI calculated for C₂₆H₂₉FN₅O₂ [M+H]⁺, 505.24, found 505.40; ¹H NMR (400 MHz, CDCl₃) δ 7.98 (d, J=4.8 Hz, 2H), 7.26 (s, 1H), 7.12 (s, 1H), 6.92-6.85 (m, 1H), 6.75-6.67 (m, 1H), 6.46 (s, 1H), 5.36 (s, 1H), 5.33-5.22 (m, 1H), 4.95 (t, J=7.4 Hz, 1H), 4.72 (t, J=8.5 Hz, 1H), 4.61 (t, J=8.3 Hz, 1H), 4.51 (t, J=9.4 Hz, 1H), 3.50 (s, 2H), 3.37-3.32 (m, 1H), 2.95 (d, J=5.9 Hz, 3H), 2.61 (s, 6H), 1.28-1.16 (m, 2H), 1.10-1.02 (m, 2H).

### Example 205: 1-((3S,5R)-1-Acryloyl-5-(methoxymethyl)pyrrolidin-3-yl)-3-((1-cyclopropyl-6-fluoro-1H-benzo[d]imidazol-5-yl)ethynyl)-5-(ethylamino)-1H-pyrazole-4-carboxamide

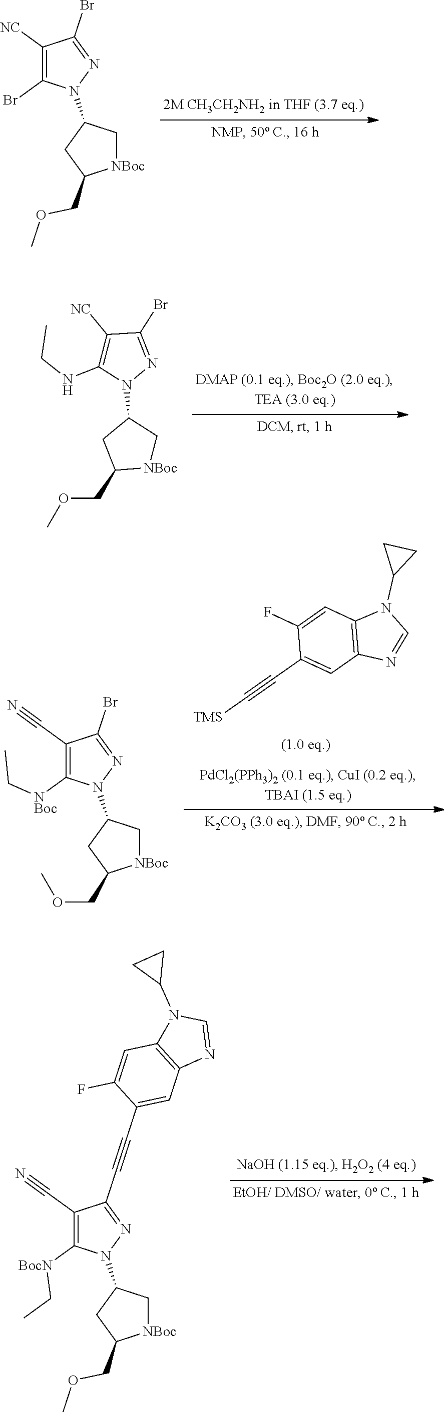 [SMILES: COC[C@H]1C[C@H](n2nc(Br)c(C#N)c2Br)CN1C(=O)OC(C)(C)C ; CCNc1c(C#N)c(Br)nn1[C@H]1C[C@H](COC)N(C(=O)OC(C)(C)C)C1 ; CCN(C(=O)OC(C)(C)C)c1c(C#N)c(Br)nn1[C@H]1C[C@H](COC)N(C(=O)OC(C)(C)C)C1 ; C[Si](C)(C)C#Cc1cc2ncn(C3CC3)c2cc1F ; CCN(C(=O)OC(C)(C)C)c1c(C#N)c(C#Cc2cc3ncn(C4CC4)c3cc2F)nn1[C@H]1C[C@H](COC)N(C(=O)OC(C)(C)C)C1]

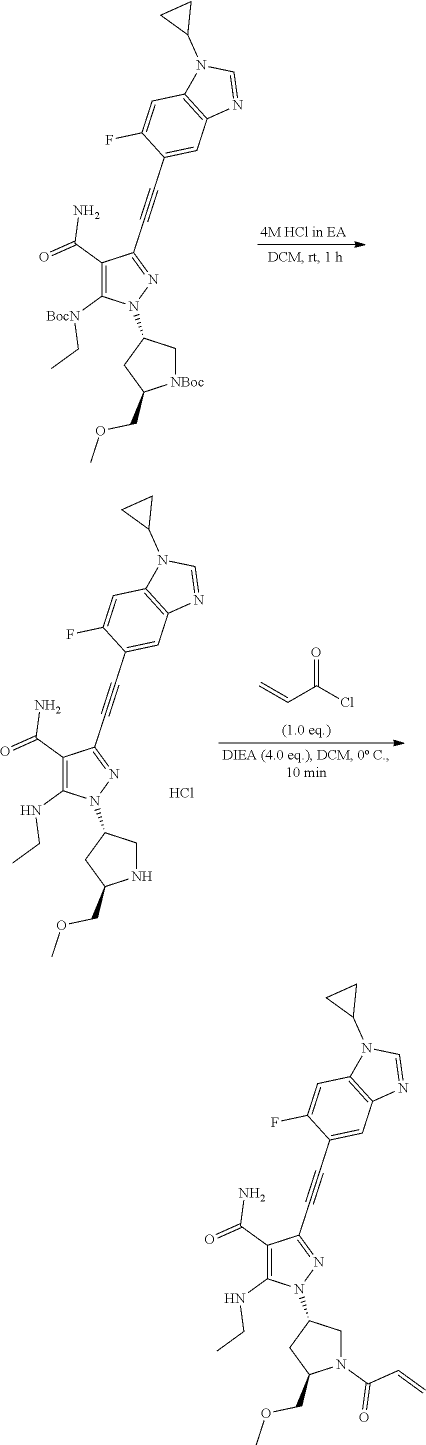

### Step 1: Tert-butyl (2R,4S)-4-[3-bromo-4-cyano-5-(ethylamino)pyrazol-1-yl]-2-(methoxymethyl)pyrrolidine-1-carboxylate

To a stirred solution of tert-butyl(2R,4S)-4-(3,5-dibromo-4-cyanopyrazol-1-yl)-2-(methoxymethyl)pyrrolidine-1-carboxylate (5.00 g, 10.77 mmol) in NMP (10.00 mL) was added ethylamine solution 2.0 M in THF (19.98 mL, 39.96 mmol) dropwise at room temperature under nitrogen atmosphere. The reaction mixture was stirred for 16 h at 50° C. under nitrogen atmosphere. The residue was purified by reverse flash chromatography with the following conditions: column: C18 silica gel; mobile phase: ACN in water (10 mmol/L NH₄HCO₃), 25% to 40% gradient in 20 min; detector: UV 254 nm. The fractions contained desired product were combined and concentrated to afford tert-butyl (2R,4S)-4-[3-bromo-4-cyano-5-(ethylamino)pyrazol-1-yl]-2-(methoxymethyl)pyrrolidine-1-carboxylate (3.80 g, 82%) as a light yellow oil. MS ESI calculated for C₁₇H₂₆BrN₅O₃ [M+H]+, 428.12, 430.12, found 428.20, 430.20.

### Step 2: Tert-butyl(2R,4S)-4-{3-bromo-5-[(tert-butoxycarbonyl)(ethyl)amino]-4-cyanopyrazol-1-yl}-2-(methoxymethyl)pyrrolidine-1-carboxylate

To a stirred solution of tert-butyl(2R,4S)-4-[3-bromo-4-cyano-5-(ethylamino)pyrazol-1-yl]-2-(methoxymethyl)pyrrolidine-1-carboxylate (3.69 g, 8.61 mmol) and TEA (2.62 g, 25.84 mmol) in DCM (37.00 mL) were added Boc₂O (3.76 g, 17.23 mmol) and DMAP (0.11 g, 0.86 mmol) in portions at 0° C. under nitrogen atmosphere. The reaction mixture was stirred for 1 h at room temperature under nitrogen atmosphere. The resulting mixture was concentrated under reduced pressure. The residue was purified by silica gel column chromatography, eluted with PE/EA (1/2). The fractions contained desired product were combined and concentrated to afford tert-butyl (2R,4S)-4-{3-bromo-5-[(tert-butoxycarbonyl)(ethyl)amino]-4-cyanopyrazol-1-yl}-2-(methoxymethyl)pyrrolidine-1-carboxylate (2.30 g, 50%) as a yellow oil. MS ESI calculated for C₂₂H₃₄BrN₅O₅ [M+H]⁺, 528.17, 530.17, found 528.20, 530.20.

### Step 3: Tert-butyl (2R,4S)-4-(5-((tert-butoxycarbonyl)(ethyl)amino)-4-cyano-3-((1-cyclopropyl-6-fluoro-1H-benzo[d]imidazol-5-yl)ethynyl)-1H-pyrazol-1-yl)-2-(methoxymethyl)pyrrolidine-1-carboxylate

To a stirred mixture of tert-butyl(2R,4S)-4-{3-bromo-5-[(tert-butoxycarbonyl)(ethyl)amino]-4-cyanopyrazol-1-yl}-2-(methoxymethyl)pyrrolidine-1-carboxylate (2.07 g, 3.91 mmol), 1-cyclopropyl-6-fluoro-5-[2-(trimethylsilyl)ethynyl]-1,3-benzodiazole (1.28 g, 4.70 mmol), CuI (0.15 g, 0.78 mmol), Pd(PPh₃)₂Cl₂ (0.27 g 0.39 mmol) and K₂CO₃ (1.62 g 11.75 mmol) in DMF (20.00 mL) was added TBAI (2.17 g, 5.87 mmol). The reaction mixture was degassed with nitrogen for three times and stirred for 2 h at 90° C. The resulting mixture was diluted with water (30 mL) and extracted with EA (3×30 mL). The combined organic layers was washed with brine (3×10 mL), dried over anhydrous Na₂SO₄, filtered and concentrated under reduced pressure. The residue was purified by silica gel column chromatography, eluted with 4% MeOH in DCM. The fractions contained desired product were combined and concentrated to afford tert-butyl (2R,4S)-4-(5-((tert-butoxycarbonyl)(ethyl)amino)-4-cyano-3-((1-cyclopropyl-6-fluoro-1H-benzo[d]imidazol-5-yl)ethynyl)-1H-pyrazol-1-yl)-2-(methoxymethyl)pyrrolidine-1-carboxylate (1.70 g, 67%) as a dark yellow solid. MS ESI calculated for C₃₄H₄₂FN₇O₅ [M+H]⁺, 648.32, found 648.70; ¹H NMR (400 MHz, CDCl₃) δ 8.02 (s, 1H), 7.67-7.63 (m, 1H), 7.55-7.52 (m, 1H), 7.46-7.41 (m, 1H), 5.00 (s, 1H), 4.22 (s, 1H), 3.70-3.69 (m, 1H), 3.35 (d, J=8.8 Hz, 4H), 2.95 (s, 1H), 2.88 (s, 1H), 2.71 (s, 1H), 2.62 (s, 1H), 2.32 (s, 1H), 2.21 (s, 1H), 1.47 (d, J=4.3 Hz, 21H), 1.19 (d, J=2.3 Hz, 2H), 1.14-1.01 (m, 2H).

Step 4: Tert-butyl (2R,4S)-4-(5-((tert-butoxycarbonyl)(ethyl)amino)-4-carbamoyl-3-((1-cyclopropyl-6-fluoro-1H-benzo[d]imidazol-5-yl)ethynyl)-1H-pyrazol-1-yl)-2-(methoxymethyl)pyrrolidine-1-carboxylate

To a stirred solution of tert-butyl (2R,4S)-4-(5-((tert-butoxycarbonyl)(ethyl)amino)-4-cyano-3-((1-cyclopropyl-6-fluoro-1H-benzo[d]imidazol-5-yl)ethynyl)-1H-pyrazol-1-yl)-2-(methoxymethyl)pyrrolidine-1-carboxylat (1.53 g, 2.36 mmol) in DMSO (2.60 mL) and EtOH (12.70 mL) were added NaOH (0.11 g, 2.71 mmol) and H₂O₂ (30%) (0.32 g, 9.44 mmol) at 0° C. The reaction mixture was stirred for 1 h at 0° C. Then the reaction mixture was warmed up to 25° C. and stirred for another 0.5 h at room temperature. The resulting mixture was diluted with water (10 mL) and extracted with EA (3×10 mL). The combined organic layers was washed with brine (5×10 mL), dried over anhydrous Na₂SO₄, filtered and concentrated under reduced pressure. The residue was purified by silica gel column chromatography, eluted with 65% EtOH in EA. The fractions contained desired product were combined and concentrated to afford tert-butyl (2R,4S)-4-(5-((tert-butoxycarbonyl)(ethyl)amino)-4-carbamoyl-3-((1-cyclopropyl-6-fluoro-1H-benzo[d]imidazol-5-yl)ethynyl)-1H-pyrazol-1-yl)-2-(methoxymethyl)pyrrolidine-1-carboxylate (0.96 g, 61%) as a dark yellow solid. MS ESI calculated for C₃₄H₄₄FN₇O₆[M+H−100]⁺, 566.33, found 566.35; ¹H NMR (400 MHz, CDCl₃) δ 8.04 (s, 1H), 7.99 (s, 1H), 7.35 (d, J=8.7 Hz, 1H), 7.19 (s, 1H), 5.62 (s, 1H), 5.20 (s, 2H), 4.23 (s, 1H), 4.04-4.01 (m, 1H), 3.72 (d, J=9.0 Hz, 2H), 3.49 (s, 1H), 2.85 (s, 1H), 2.62 (s, 3H), 2.58 (s, 1H), 2.30 (s, 1H), 2.22 (s, 1H), 1.59-1.41 (m, 13H), 1.37 (d, J=3.3 Hz, 8H), 1.15-1.03 (m, 2H), 0.92-0.81 (m, 2H).

### Step 5: 3-((1-Cyclopropyl-6-fluoro-1H-benzo[d]imidazol-5-yl)ethynyl)-5-(ethylamino)-1-((3S,5R)-5-(methoxymethyl)pyrrolidin-3-yl)-1H-pyrazole-4-carboxamide hydrochloride

To a stirred solution of tert-butyl (2R,4S)-4-(5-((tert-butoxycarbonyl)(ethyl)amino)-4-carbamoyl-3-((1-cyclopropyl-6-fluoro-1H-benzo[d]imidazol-5-yl)ethynyl)-1H-pyrazol-1-yl)-2-(methoxymethyl)pyrrolidine-1-carboxylate (0.50 g, 0.75 mmol) and DCM (5.00 mL) in 4 M HCl (g) in EA (4.17 mL, 16.68 mmol) was stirred for 1 h at room temperature under argon atmosphere. The resulting mixture was concentrated under reduced pressure. The residue was purified by trituration with DCM/PE (1/5, 500 mL) to afford 3-((1-cyclopropyl-6-fluoro-1H-benzo[d]imidazol-5-yl)ethynyl)-5-(ethylamino)-1-((3S,5R)-5-(methoxymethyl)pyrrolidin-3-yl)-1H-pyrazole-4-carboxamide hydrochloride (0.61 g, crude) as a light yellow solid. MS ESI calculated for C₂₄H₂₈FN₇O₂[M+H]⁺, 466.23, found 466.35.

### Step 6: 1-((3S,5R)-1-Acryloyl-5-(methoxymethyl)pyrrolidin-3-yl)-3-((1-cyclopropyl-6-fluoro-1H-benzo[d]imidazol-5-yl)ethynyl)-5-(ethylamino)-1H-pyrazole-4-carboxamide

To a stirred mixture of 3-((1-cyclopropyl-6-fluoro-1H-benzo[d]imidazol-5-yl)ethynyl)-5-(ethylamino)-1-((3S,5R)-5-(methoxymethyl)pyrrolidin-3-yl)-1H-pyrazole-4-carboxamide hydrochloride (0.20 g, 0.43 mmol) and 2.5 M K₂CO₃ (0.71 mL, 5.13 mmol) was added acryloyl chloride (38.88 mg, 0.43 mmol) in THF (0.40 mL) dropwise at 0° C. The reaction mixture was stirred for 10 min at 0° C. The resulting mixture was extracted with EA (3×10 mL). The combined organic layers was washed with brine (2×5 mL), dried over anhydrous Na₂SO₄. After filtration, the filtrate was concentrated under reduced pressure. The residue was purified by silica gel column chromatography, eluted with CH₂Cl₂/MeOH (20/1). The fractions contained desired product were combined and concentrated to afford 1-((3S,5R)-1-acryloyl-5-(methoxymethyl)pyrrolidin-3-yl)-3-((1-cyclopropyl-6-fluoro-1H-benzo[d]imidazol-5-yl)ethynyl)-5-(ethylamino)-1H-pyrazole-4-carboxamide (74.50 mg, 33%) as an off-white solid. MS ESI calculated for C₂₇H₃₀FN₇O₃[M+H]⁺, 520.24, found 520.20; ¹H NMR (400 MHz, CDCl₃) δ 8.10-7.84 (m, 2H), 7.31 (d, J=9.0 Hz, 1H), 7.11 (s, 1H), 6.85-6.07 (m, 3H), 5.76-5.66 (m, 1H), 5.87-4.90 (m, 2H), 4.56 (d, J=9.1 Hz, 1H), 4.04 (m, 3H), 3.52-3.40 (m, 1H), 3.37 (d, J=3.9 Hz, 4H), 3.35-3.21 (m, 2H), 2.70-2.30 (m, 2H), 1.30 (t, J=7.2 Hz, 3H), 1.21-1.02 (m, 4H).

### Example 206: 5-Amino-3-[2-(1-cyclopropyl-6-fluoro-2-methyl-1,3-benzodiazol-5-yl)ethynyl]-1-[(3S,5R)-5-(methoxymethyl)-1-(prop-2-enoyl)pyrrolidin-3-yl]pyrazole-4-carboxamide

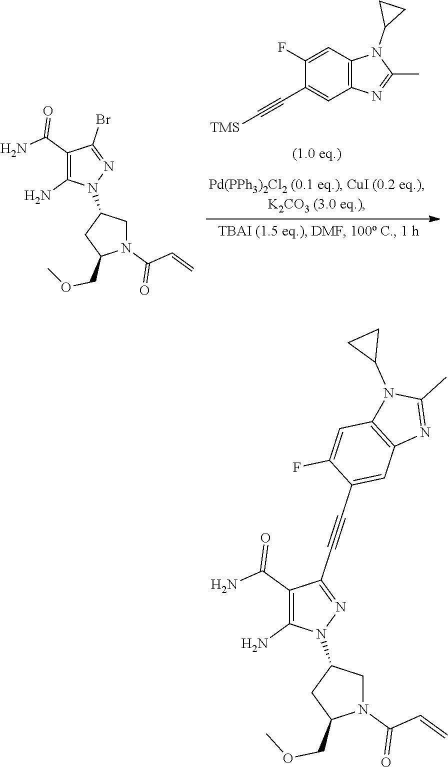 [SMILES: C=CC(=O)N1C[C@@H](n2nc(Br)c(C(N)=O)c2N)C[C@@H]1COC ; Cc1nc2cc(C#C[Si](C)(C)C)c(F)cc2n1C1CC1 ; C=CC(=O)N1C[C@@H](n2nc(C#Cc3cc4nc(C)n(C5CC5)c4cc3F)c(C(N)=O)c2N)C[C@@H]1COC]

5-amino-3-[2-(1-cyclopropyl-6-fluoro-2-methyl-1,3-benzodiazol-5-yl)ethynyl]-1-[(3S,5R)-5-(methoxymethyl)-1-(prop-2-enoyl)pyrrolidin-3-yl]pyrazole-4-carboxamide. MS ESI calculated for C₂₆H₂₈FN₇ [M+H]⁺, 506.55, found 506.40; ¹H NMR (400 MHz, CDCL₃) δ 7.90-7.85 (m, 1H), 7.40-7.32 (m, 1H), 7.10-7.07 (s, 1H), 6.61-6.35 (m, 2H), 5.80-5.74 (m, 1H), 5.50 (m, 3H), 5.10-4.88 (m, 1H), 4.65-4.42 (m, 1H), 4.10-3.93 (m, 2H), 3.90-3.89 (m, 1H), 3.56-3.45 (m, 1H), 3.40-3.35 (m, 3H), 3.32-3.30 (s, 1H), 3.02-2.75 (m, 3H), 2.68-2.58 (m, 1H), 2.40-2.39 (m, 1H), 1.42-1.23 (m, 2H), 1.20-1.13 (s, 2H).

### Example 207: 5-Amino-3-[2-(1-cyclopropyl-6-fluoro-1,3-benzodiazol-5-yl)ethynyl]-1-[(3S,5R)-5-(hydroxymethyl)-1-(prop-2-enoyl)pyrrolidin-3-yl]pyrazole-4-carboxamide

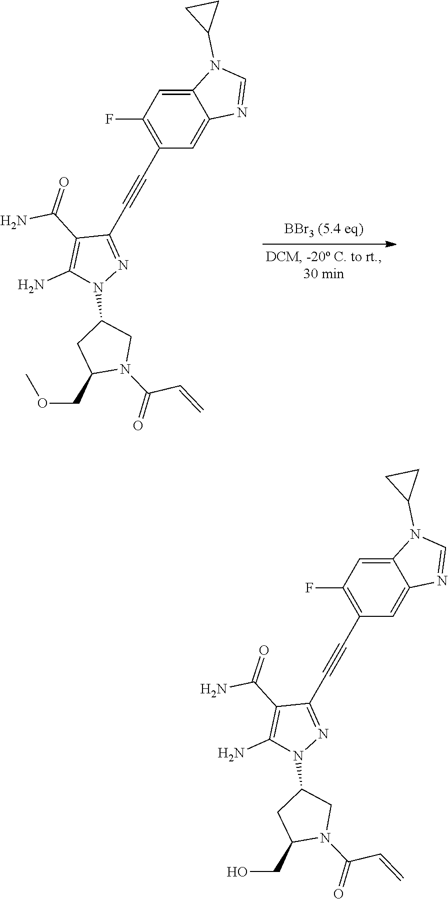 [SMILES: C=CC(=O)N1C[C@@H](n2nc(C#Cc3cc4ncn(C5CC5)c4cc3F)c(C(N)=O)c2N)C[C@@H]1COC ; C=CC(=O)N1C[C@@H](n2nc(C#Cc3cc4ncn(C5CC5)c4cc3F)c(C(N)=O)c2N)C[C@@H]1CO]

5-amino-3-[2-(1-cyclopropyl-6-fluoro-1,3-benzodiazol-5-yl)ethynyl]-1-[(3S,5R)-5-(hydroxymethyl)-1-(prop-2-enoyl)pyrrolidin-3-yl]pyrazole-4-carboxamide (79.60 mg, 45%) as an off-white solid. MS ESI calculated for C₂₄H₂₄FN₇O₃ [M+H]⁺, 478.20, found, 478.35; ¹H NMR (400 MHz, DMSO-d₆) S 8.36 (s, 1H), 7.96 (d, J=6.5 Hz, 1H), 7.65 (d, J=9.4 Hz, 1H), 7.34 (s, 1H), 7.05-6.37 (m, 4H), 6.18-6.14 (m, 1H), 5.69-5.67 (m, 1H), 5.13-5.11 (m, 1H), 5.06-4.88 (m, 1H), 4.32 (d, J=19.2 Hz, 1H), 4.05-3.84 (m, 2H), 3.75-3.50 (m, 3H), 2.39 (s, 2H), 1.12-1.09 (m, 2H), 1.06 (t, J=4.4 Hz, 2H).

### Example 208: 5-Amino-3-[2-(6-chloro-1-cyclopropyl-1,3-benzodiazol-5-yl)ethynyl]-1-[1-(prop-2-enoyl)azetidin-3-yl]pyrazole-4-carboxamide

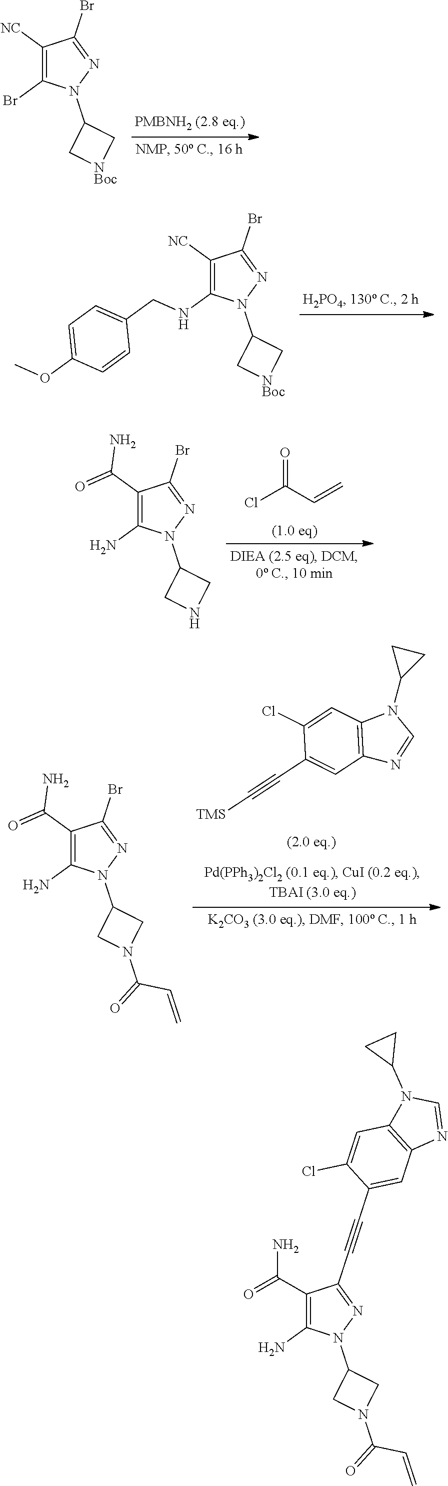

5-amino-3-[2-(6-chloro-1-cyclopropyl-1,3-benzodiazol-5-yl)ethynyl]-1-[1-(prop-2-enoyl)azetidin-3-yl]pyrazole-4-carboxamide (64.20 mg, 55%) as an off-white solid. MS ESI calculated for C₂₂H₂₀ClN₇O₂[M+H]⁺, 450.14, found 450.30; ¹H NMR (400 MHz, DMSO-d₆) δ 8.40 (s, 1H), 8.07 (s, 1H), 7.89 (s, 1H), 7.39 (s, 1H), 6.74 (s, 1H), 6.64 (s, 2H), 6.43-6.39 (m, 1H), 6.16-6.10 (m, 1H), 5.72-5.68 (m, 1H), 5.32-5.21 (m, 1H), 4.65 (t, J=8.6 Hz, 1H), 4.51 (t, J=7.2 Hz, 1H), 4.35 (t, J=9.3 Hz, 1H), 4.23-4.12 (m, 1H), 3.68-3.54 (m, 1H), 1.13-0.99 (m, 4H).

### Example 209: 5-Amino-3-[2-(6-chloro-1-ethyl-1,3-benzodiazol-5-yl)ethynyl]-1-[1-(prop-2-enoyl)azetidin-3-yl]pyrazole-4-carboxamide

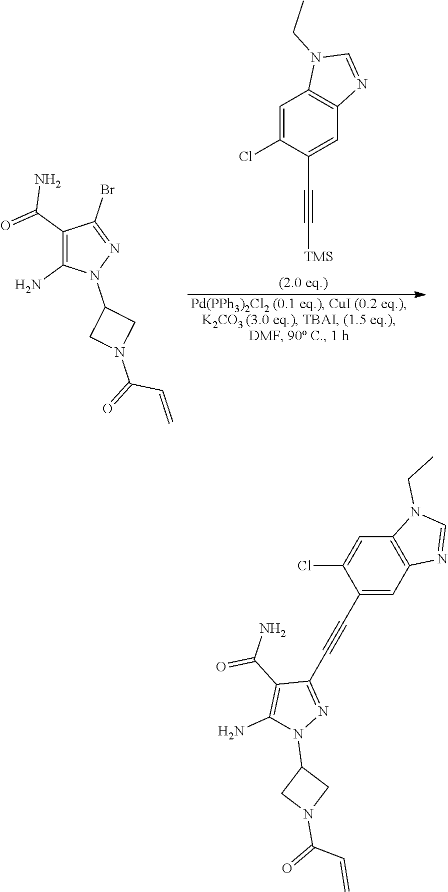

5-amino-3-[2-(6-chloro-1-ethyl-1,3-benzodiazol-5-yl)ethynyl]-1-[1-(prop-2-enoyl)azetidin-3-yl]pyrazole-4-carboxamide. MS ESI calculated for C₂₁H₂₀ClN₇O₂[M+H]⁺, 438.14, found 438.05; ¹H NMR (400 MHz, DMSO-d₆) δ 8.43 (s, 1H), 8.06 (s, 1H), 8.01 (s, 1H), 7.38 (s, 1H), 6.75 (s, 1H), 6.64 (s, 2H), 6.42-6.33 (m, 1H), 6.22-6.16 (m, 1H), 5.65-5.72 (m, 1H), 5.28 (q, J=7.6, 6.6 Hz, 1H), 4.63 (d, J=8.5 Hz, 1H), 4.53-4.46 (m, 1H), 4.31 (q, J=7.3 Hz, 3H), 4.22 (d, J=5.6 Hz, 1H), 1.42 (t, J=7.2 Hz, 3H).

### Example 210: 5-Amino-3-[2-(1-cyclopropyl-4,6-difluoro-1,3-benzodiazol-5-yl)ethynyl]-1-[1-(prop-2-enoyl)azetidin-3-yl]pyrazole-4-carboxamide

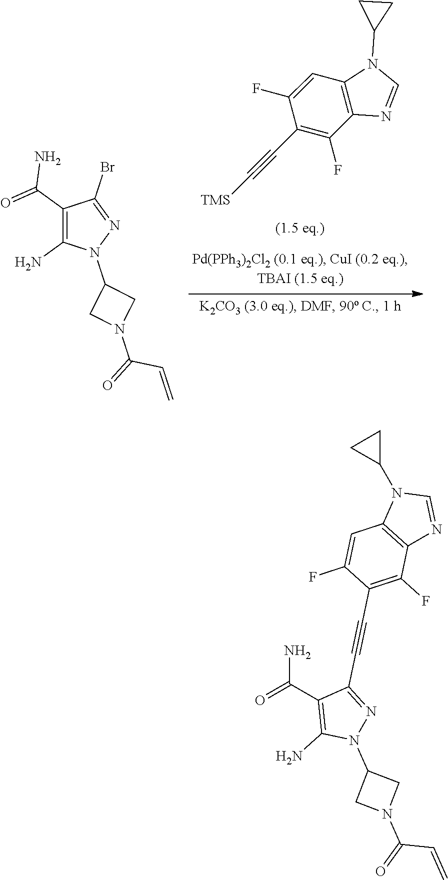 [SMILES: C=CC(=O)N1CC(n2nc(Br)c(C(N)=O)c2N)C1 ; C[Si](C)(C)C#Cc1c(F)cc2c(ncn2C2CC2)c1F ; C=CC(=O)N1CC(n2nc(C#Cc3c(F)cc4c(ncn4C4CC4)c3F)c(C(N)=O)c2N)C1]

5-amino-3-[2-(1-cyclopropyl-4,6-difluoro-1,3-benzodiazol-5-yl)ethynyl]-1-[1-(prop-2-enoyl)azetidin-3-yl]pyrazole-4-carboxamide. MS ESI calculated for C₂₂H₁₉F₂N₇O₂[M+H]⁺, 452.16, found 452.35; ¹H NMR (400 MHz, DMSO-d₆) δ 8.42 (s, 1H), 7.60 (d, J=8.8 Hz, 1H), 7.48 (s, 1H), 6.67 (s, 3H), 6.43-6.38 (m, 1H), 6.16-6.12 (m, 1H), 5.74-5.71 (m, 1H), 5.32-5.25 (m, 1H), 4.64 (m, 1H), 4.56-4.52 (m, 1H), 4.35 (t, J=9.2 Hz, 1H), 4.27-4.21 (m, 1H), 3.57-3.52 (m, 1H), 1.19-1.02 (m, 4H).

### Example 211: 5-Amino-3-[2-(1-cyclopropyl-6-fluoro-1,3-benzodiazol-5-yl)ethynyl]-1-[(3S,5R)-5-[(difluoromethoxy)methyl]-1-(prop-2-enoyl)pyrrolidin-3-yl]pyrazole-4-carboxamide

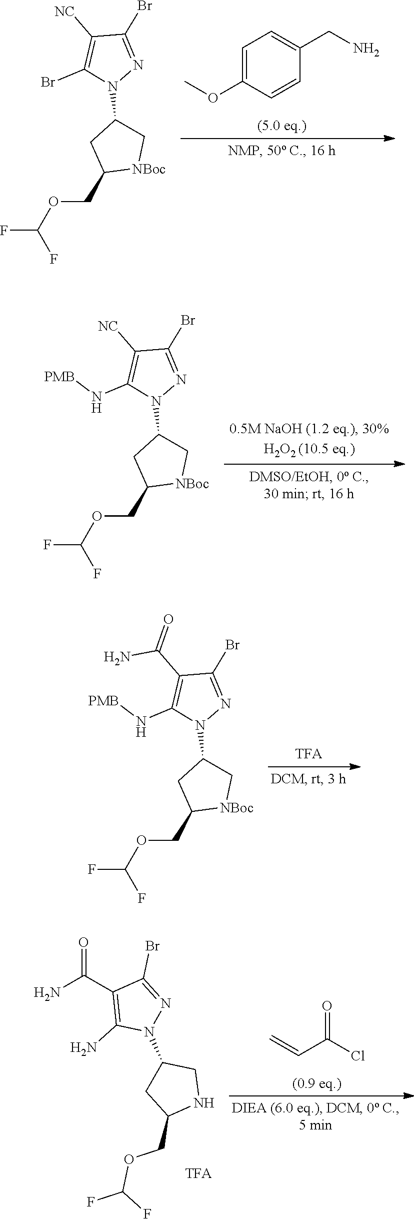

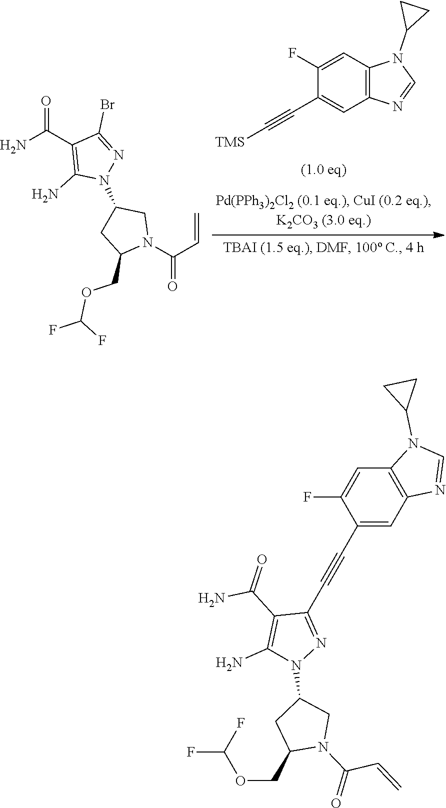 [SMILES: C=CC(=O)N1C[C@@H](n2nc(Br)c(C(N)=O)c2N)C[C@@H]1COC(F)F ; C[Si](C)(C)C#Cc1cc2ncn(C3CC3)c2cc1F ; C=CC(=O)N1C[C@@H](n2nc(C#Cc3cc4ncn(C5CC5)c4cc3F)c(C(N)=O)c2N)C[C@@H]1COC(F)F]

5-amino-3-[2-(1-cyclopropyl-6-fluoro-1,3-benzodiazol-5-yl)ethynyl]-1-[(3S,5R)-5-[(difluoromethoxy)methyl]-1-(prop-2-enoyl)pyrrolidin-3-yl]pyrazole-4-carboxamide. MS ESI calculated for C₂₅H₂₄F₃N₇O₃[M+H]⁺, 528.19, found 528.40; ¹H NMR (400 MHz, CDCl₃) δ 8.08 (s, 2H), 7.48 (s, 1H), 7.07 (s, 1H), 6.60-5.98 (m, 3H), 5.76 (t, J=6.1 Hz, 1H), 5.48 (d, J=58.1 Hz, 3H), 4.96 (q, J=8.0 Hz, 1H), 4.68 (d, J=9.2 Hz, 1H), 4.53-4.36 (m, 1H), 4.09-4.06 (m, 2H), 3.98 (d, J=9.9 Hz, 1H), 3.41 (s, 1H), 2.84-2.72 (m, 1H), 2.49-2.31 (m, J=13.0, 7.1 Hz, 1H), 1.24 (d, J=6.7 Hz, 2H), 1.09 (s, 2H).

### Example 212: 5-Amino-3-[2-(1-cyclopropyl-1,3-benzodiazol-5-yl)ethynyl]-1-[(3S,5R)-5-[(difluoromethoxy)methyl]-1-(prop-2-enoyl)pyrrolidin-3-yl]pyrazole-4-carboxamide

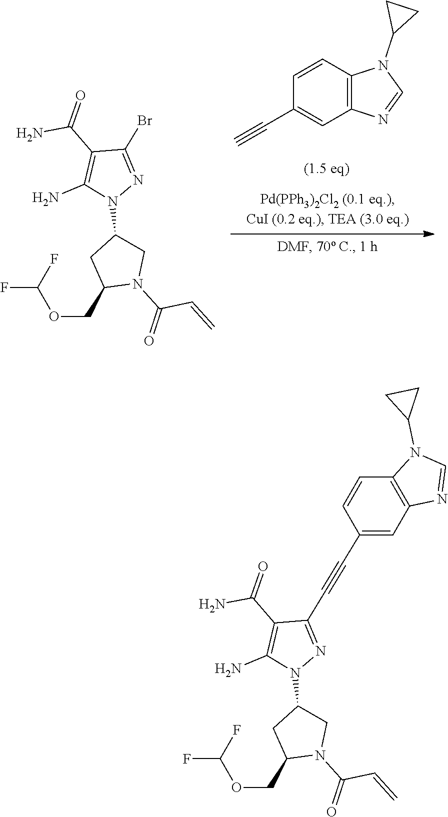 [SMILES: C=CC(=O)N1C[C@@H](n2nc(Br)c(C(N)=O)c2N)C[C@@H]1COC(F)F ; C#Cc1ccc2c(c1)ncn2C1CC1 ; C=CC(=O)N1C[C@@H](n2nc(C#Cc3ccc4c(c3)ncn4C3CC3)c(C(N)=O)c2N)C[C@@H]1COC(F)F]

5-amino-3-[2-(1-cyclopropyl-1,3-benzodiazol-5-yl)ethynyl]-1-[(3S,5R)-5-[(difluoromethoxy)methyl]-1-(prop-2-enoyl)pyrrolidin-3-yl]pyrazole-4-carboxamide. MS ESI calculated for C₂₅H₂₅F₂N₇O₃[M+H]⁺, 510.20, found 510.35; ¹H NMR (400 MHz, CDCl₃) δ 8.11 (s, 1H), 8.03 (s, 1H), 7.63-7.51 (m, 2H), 6.90 (s, 1H), 6.54-6.40 (m, 2H), 6.26 (s, 1H), 5.75 (t, J=6.1 Hz, 1H), 5.48 (d, J=51.1 Hz, 3H), 5.02-4.93 (m, 1H), 4.68 (d, J=9.0 Hz, 1H), 4.51-4.36 (m, 1H), 4.14-3.84 (m, 3H), 3.52-3.34 (m, 1H), 2.84-2.72 (m, 1H), 2.51-2.31 (m, 1H), 1.29-1.17 (m, 2H), 1.20-1.07 (m, 2H).

### Example 213: 3-[2-(1-Cyclopropyl-6-fluoro-1,3-benzodiazol-5-yl)ethynyl]-5-(methylamino)-1-{[1-(prop-2-enoyl)azetidin-3-yl]methyl}pyrazole-4-carboxamide

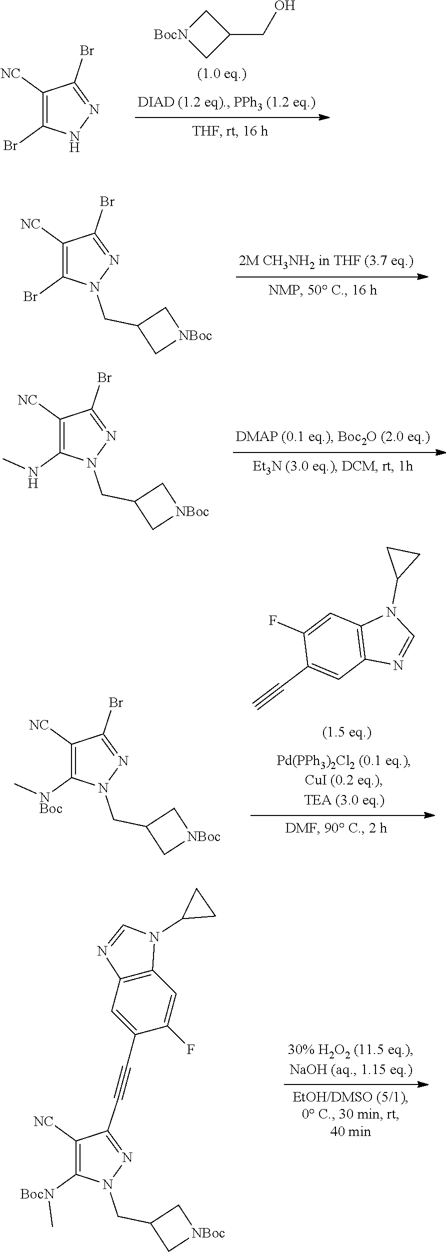

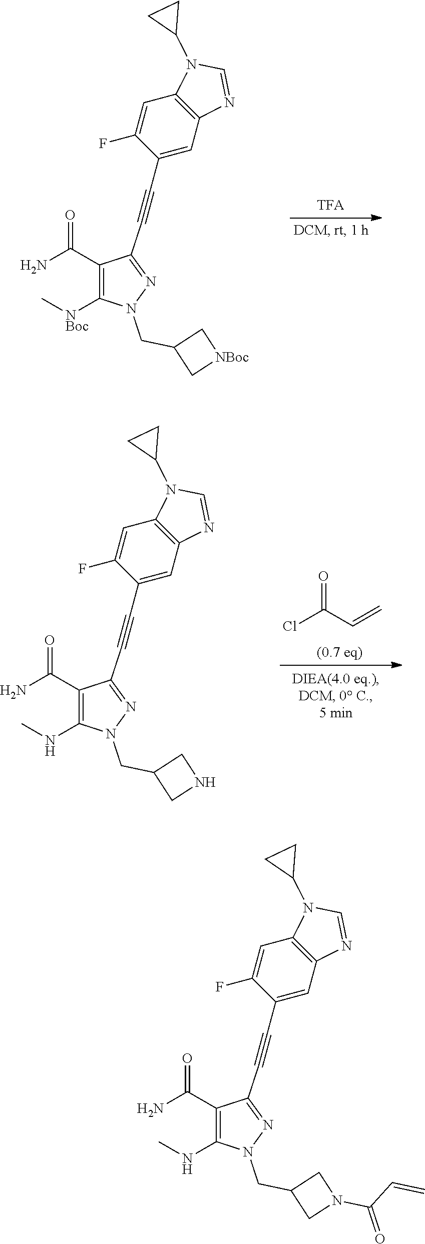

3-[2-(1-cyclopropyl-6-fluoro-1,3-benzodiazol-5-yl)ethynyl]-5-(methylamino)-1-{[1-(prop-2-enoyl)azetidin-3-yl]methyl}pyrazole-4-carboxamide. MS ESI calculated for C₂₄H₂₄FN₇O₂ [M+H]+, 462.20, found 462.30; ¹H NMR (400 MHz, CDCl₃) δ 8.01-7.96 (m, 2H), 7.33 (d, J=8 Hz, 1H), 7.12 (s, 1H), 6.76 (s, 1H), 6.39-6.34 (m, 1H), 6.24-6.16 (m, 1H), 5.71-5.68 (m, 1H), 5.36 (s, 1H), 4.40-4.34 (m, 3H), 4.28-4.23 (m, 1H), 4.19-4.15 (m, 1H), 3.93-3.89 (m, 1H), 3.42-3.37 (m, 1H), 3.33-3.29 (m, 1H), 3.02 (s, 3H), 1.25-1.18 (m, 2H), 1.11-1.07 (in, 2H).

### Example 214: 5-Amino-3-[2-(6-chloro-1-cyclopropyl-2-methyl-1,3-benzodiazol-5-yl)ethynyl]-1-[(3 S,5R)-5-(methoxymethyl)-1-(prop-2-enoyl)pyrrolidin-3-yl]pyrazole-4-carboxamide

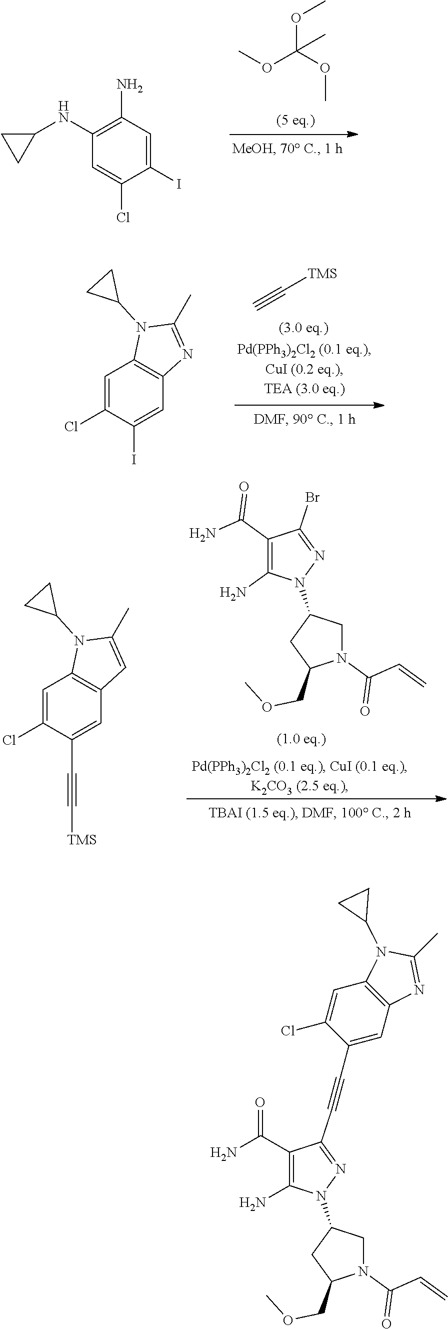

5-amino-3-[2-(6-chloro-1-cyclopropyl-2-methyl-1,3-benzodiazol-5-yl)ethynyl]-1-[(3S,5R)-5-(methoxymethyl)-1-(prop-2-enoyl)pyrrolidin-3-yl]pyrazole-4-carboxamide. MS ESI calculated for C₂₆H₂₈ClN₇O₃[M+H]+, 522.19, found 522.15; ¹H NMR (400 MHz, CDCl₃) δ 7.92 (d, J=5.0 Hz, 1H), 7.56 (s, 1H), 7.20 (s, 1H), 6.58-6.35 (m, 2H), 5.76-5.62 (m, 3H), 5.55 (s, 1H), 5.17-4.88 (m, 1H), 4.62-4.37 (m, 1H), 4.11-3.94 (m, 2H), 3.89-3.80 (m, 1H), 3.47-3.39 (m, 1H), 3.38 (d, J=2.0 Hz, 3H), 3.22-3.14 (m, 1H), 2.68 (s, 4H), 2.37-2.28 (m, 1H), 1.29-1.20 (m, 2H), 1.11-1.03 (in, 2H).

### Example 215: 5-Amino-3-[2-(1-cyclopropyl-4,6-difluoro-1,3-benzodiazol-5-yl)ethynyl]-1-[(3 S,5R)-5-[(difluoromethoxy)methyl]-1-(prop-2-enoyl)pyrrolidin-3-yl]pyrazole-4-carboxamide

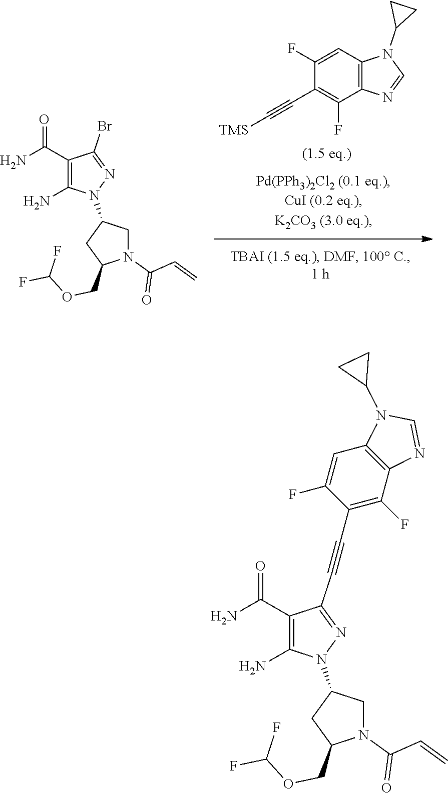 [SMILES: C=CC(=O)N1C[C@@H](n2nc(Br)c(C(N)=O)c2N)C[C@@H]1COC(F)F ; C[Si](C)(C)C#Cc1c(F)cc2c(ncn2C2CC2)c1F ; C=CC(=O)N1C[C@@H](n2nc(C#Cc3c(F)cc4c(ncn4C4CC4)c3F)c(C(N)=O)c2N)C[C@@H]1COC(F)F]

5-amino-3-[2-(1-cyclopropyl-4,6-difluoro-1,3-benzodiazol-5-yl)ethynyl]-1-[(3S,5R)-5-[(difluoromethoxy)methyl]-1-(prop-2-enoyl)pyrrolidin-3-yl]pyrazole-4-carboxamide. MS ESI calculated for C₂₅H₂₃F₄N₇O₃[M+H]⁺, 546.18, found 546.15; ¹H NMR (400 MHz, CDCl₃) δ 7.97-7.80 (s, 1H), 7.19-7.18 (m, 1H), 7.10-6.90 (m, 1H), 6.44-6.40 (m, 2H), 6.30-6.10 (m, 1H), 5.83-5.70 (m, 1H), 5.60-5.49 (m, 3H), 4.95-4.90 (m, 1H), 4.70-4.68 (m, 1H), 4.42-4.40 (m, 1H), 4.17-3.93 (m, 3H), 3.39-3.30 (m, 1H), 2.85-2.71 (m, 1H), 2.39-2.35 (m, 1H), 1.25-1.20 (m, 2H), 1.10-1.05 (m, 2H).

### Example 216: 3-[2-(1-Cyclopropyl-6-fluoro-1,3-benzodiazol-5-yl)ethynyl]-5-(cyclopropylamino)-1-[(3S,5R)-5-(methoxymethyl)-1-(prop-2-enoyl)pyrrolidin-3-yl]pyrazole-4-carboxamide

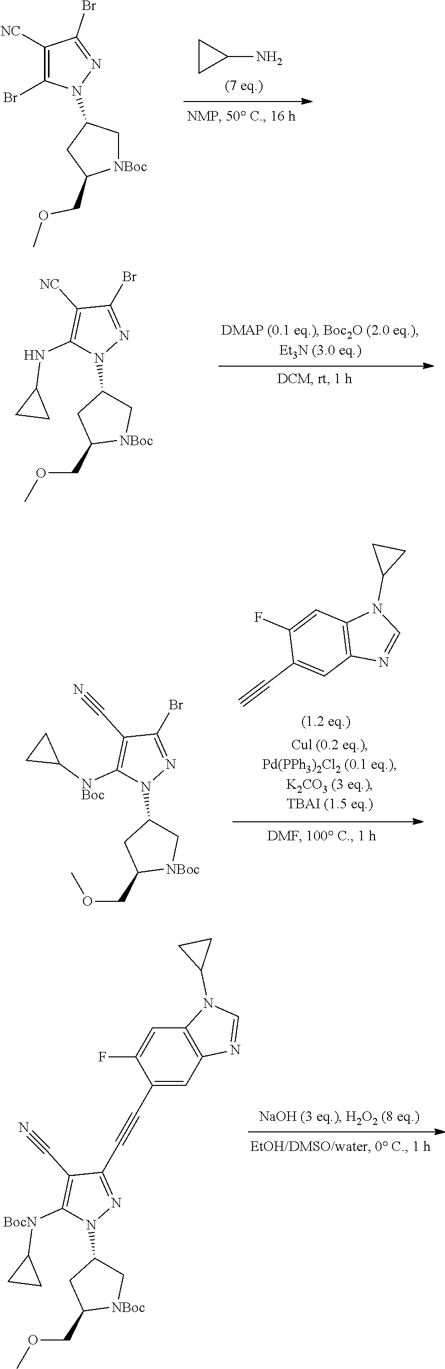

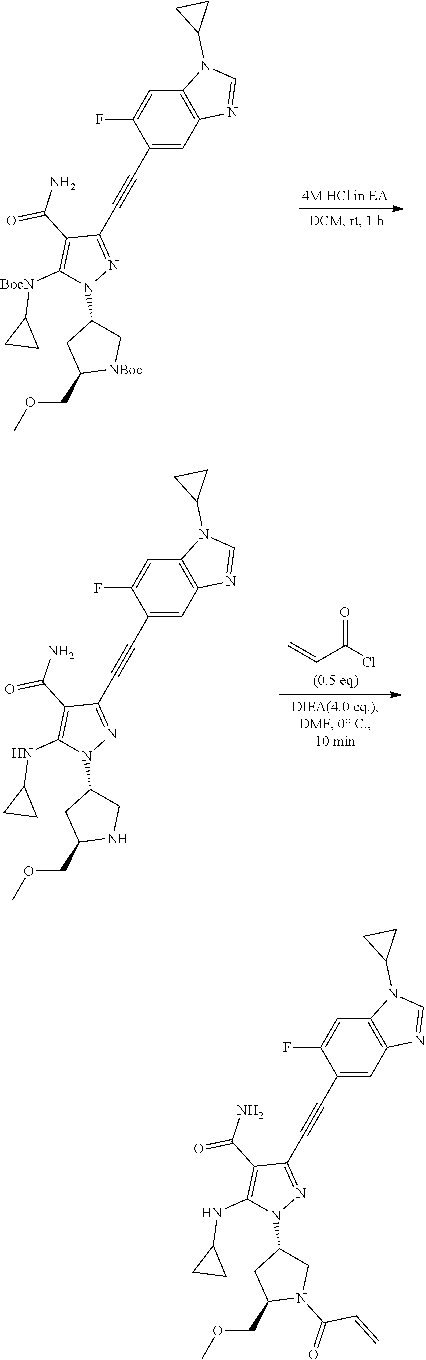

3-[2-(1-cyclopropyl-6-fluoro-1,3-benzodiazol-5-yl)ethynyl]-5-(cyclopropylamino)-1-[(3S,5R)-5-(methoxymethyl)-1-(prop-2-enoyl)pyrrolidin-3-yl]pyrazole-4-carboxamide. MS ESI calculated for C₂₉H₃₀FN₆O₃[M+H]⁺, 532.24, found 532.45; ¹H NMR (400 MHz, CDCl₃) δ 7.97 (d, J=5.8 Hz, 2H), 7.40 (d, J=2.3 Hz, 1H), 7.34 (d, J=8.5 Hz, 1H), 7.10 (s, 1H), 6.56-6.39 (m, 2H), 6.15-5.84 (m, 1H), 5.76-5.69 (m, 1H), 5.34 (s, 1H), 4.59-4.57 (m, 1H), 4.13-3.99 (m, 2H), 3.91-3.87 (m, 1H), 3.54-3.44 (m, 1H), 3.36 (d, J=10.4 Hz, 4H), 3.01-2.74 (m, 2H), 2.35-2.32 (m, 1H), 1.24-1.16 (m, 2H), 1.09-1.05 (m, 2H), 0.94-0.83 (m, 2H), 0.81-0.73 (m, 2H).

### Example 217: 3-[2-(1-cyclopropyl-6-fluoro-1,3-benzodiazol-5-yl)ethynyl]-1-[(3S,5R)-5-(2-hydroxypropan-2-yl)-1-(prop-2-enoyl)pyrrolidin-3-yl]-5-(methylamino)pyrazole-4-carboxamide

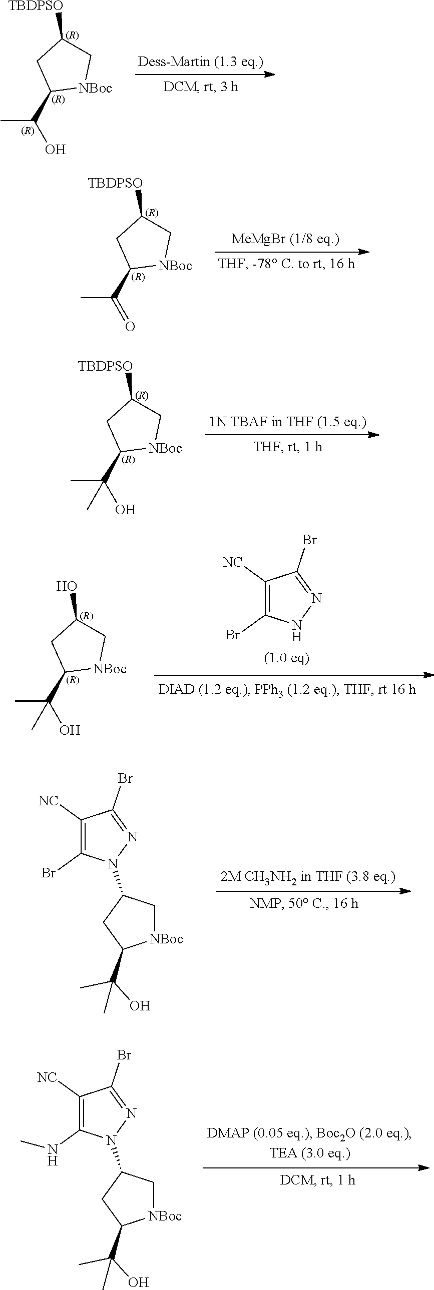

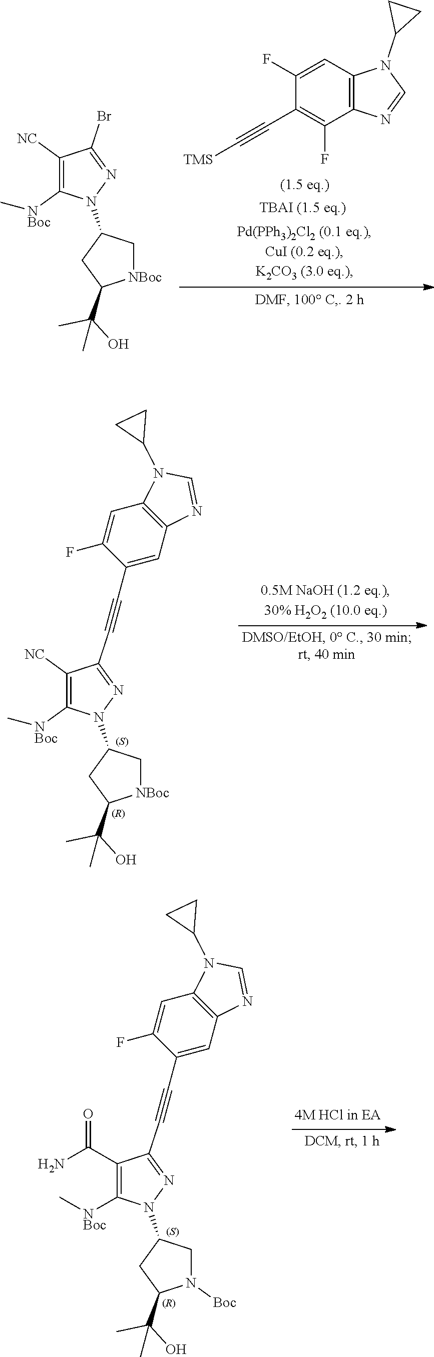

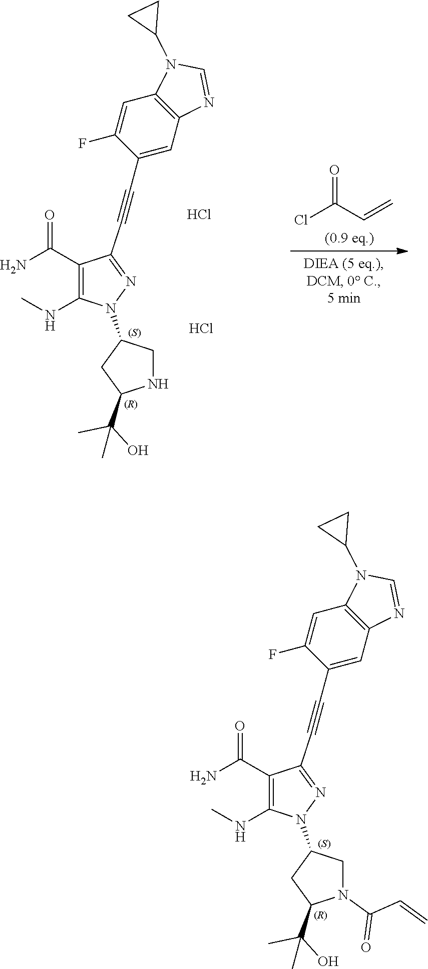

3-[2-(1-cyclopropyl-6-fluoro-1,3-benzodiazol-5-yl)ethynyl]-1-[(3S,5R)-5-(2-hydroxypropan-2-yl)-1-(prop-2-enoyl)pyrrolidin-3-yl]-5-(methylamino)pyrazole-4-carboxamide. MS ESI calculated for C₂₇H₃₀FN₇O₃[M+H]⁺, 520.24, found 520.20; ¹H NMR (400 MHz, CDCl₃) δ 8.36 (s, 1H), 7.95 (d, J=4.6 Hz, 1H), 7.64 (d, J=4.9 Hz, 1H), 7.47 (s, 1H), 6.72-6.59 (m, 3H), 6.18-6.13 (m, 1H), 5.71-5.68 (m, 1H), 5.41-5.35 (m, 1H), 4.96 (s, 1H), 4.40-4.31 (m, 1H), 4.19-4.15 (m, 3H), 4.09-3.49 (m, 3H), 2.48-2.35 (m, 2H), 1.17-1.03 (m, 10H).

### Example 218: 3-[2-(1-Cyclopropyl-6-fluoro-1,3-benzodiazol-5-yl)ethynyl]-1-[(3S,5R)-5-[(1R)-1-hydroxyethyl]-1-(prop-2-enoyl)pyrrolidin-3-yl]-5-(methylamino)pyrazole-4-carboxamide

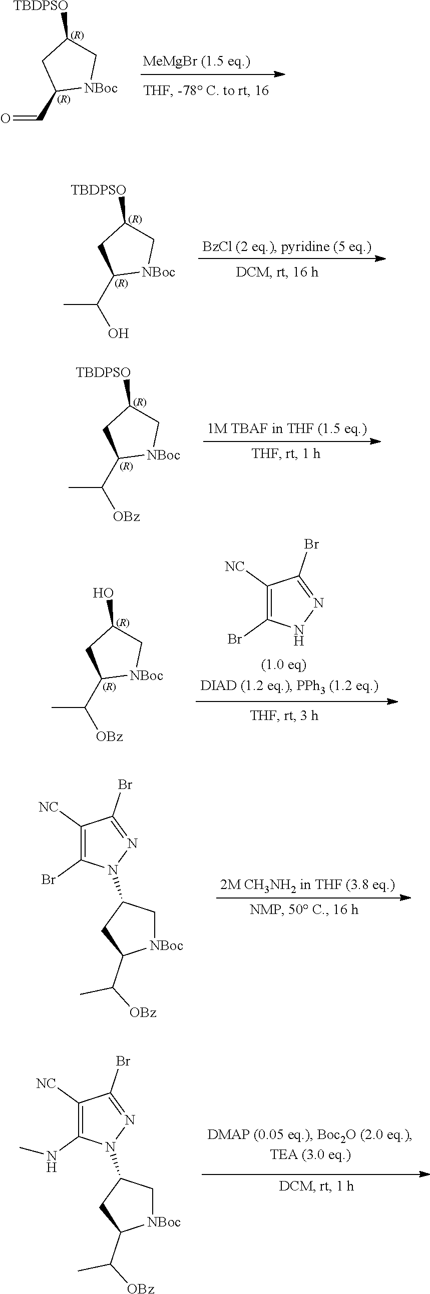

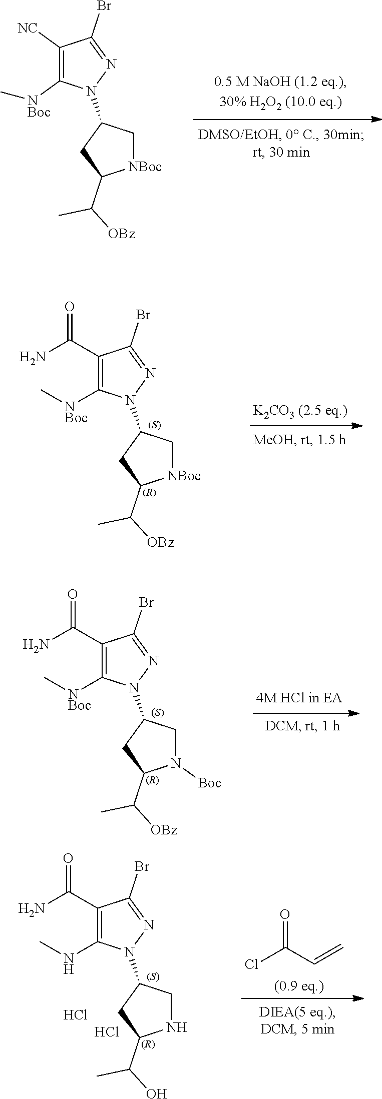

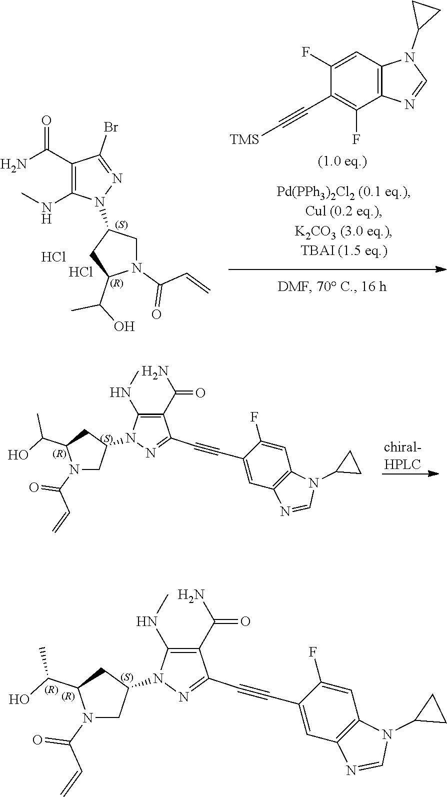

3-[2-(1-cyclopropyl-6-fluoro-1,3-benzodiazol-5-yl)ethynyl]-1-[(3S,5R)-5-[(1R)-1-hydroxyethyl]-1-(prop-2-enoyl)pyrrolidin-3-yl]-5-(methylamino)pyrazole-4-carboxamide. MS ESI calculated for C₂₆H₂₈FN₇O₃[M+H]⁺, 506.22, found 506.45; ¹H NMR (400 MHz, DMSO-d₆) δ 8.36 (s, 1H), 7.95-7.91 (m, 1H), 7.64 (d, J=9.4 Hz, 1H), 7.49 (s, 1H), 6.76 (s, 1H), 6.74-6.64 (m, 1H), 6.67-6.54 (m, 1H), 6.22-6.10 (m, 1H), 5.68-5.60 (m, 1H), 5.33-5.30 (m, 1H), 4.90 (d, J=4.5 Hz, 1H), 4.16 (d, J=7.5 Hz, 1H), 4.03-4.01 (m, 1H), 3.92-3.73 (m, 2H), 3.52-3.50 (m, 1H), 2.95 (t, J=5.7 Hz, 3H), 2.47-2.20 (m, 2H), 1.17-1.01 (m, 7H).

### Example 219: 3-[2-(1-Cyclopropyl-6-fluoro-1,3-benzodiazol-5-yl)ethynyl]-1-[(3S,5R)-5-[(1S)-1-hydroxyethyl]-1-(prop-2-enoyl)pyrrolidin-3-yl]-5-(methylamino)pyrazole-4-carboxamide

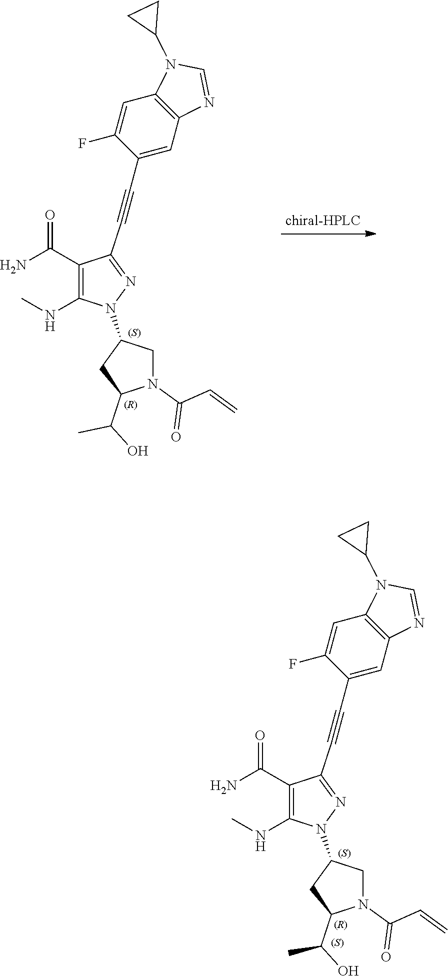

3-[2-(1-cyclopropyl-6-fluoro-1,3-benzodiazol-5-yl)ethynyl]-1-[(3S,5R)-5-[(1S)-1-hydroxyethyl]-1-(prop-2-enoyl)pyrrolidin-3-yl]-5-(methylamino)pyrazole-4-carboxamide. MS ESI calculated for C₂₆H₂₈FN₇O₃[M+H]⁺, 506.22, found 506.40; ¹H NMR (400 MHz, DMSO-d₆) δ 8.36 (s, 1H), 7.95-7.90 (m, 1H), 7.64 (d, J=9.4 Hz, 1H), 7.48 (s, 1H), 6.88-6.50 (m, 3H), 6.14-6.10 (m, 1H), 5.66-5.62 (m, 1H), 5.33-5.18 (m, 1H), 4.99 (d, J=19.8 Hz, 1H), 4.50-4.17 (m, 1H), 3.99-3.96 (m, 2H), 3.70-3.69 (m, 1H), 3.52-3.50 (m, 1H), 2.98-2.92 (m, 3H), 2.44-2.24 (m, 2H), 1.15-0.98 (m, 7H).

### Example 220: 1-((3S,5R)-1-Acryloyl-5-((R)-1-hydroxyethyl)pyrrolidin-3-yl)-3-((6-chloro-1-cyclopropyl-2-methyl-1H-benzo[d]imidazol-5-yl)ethynyl)-5-(methylamino)-1H-pyrazole-4-carboxamide

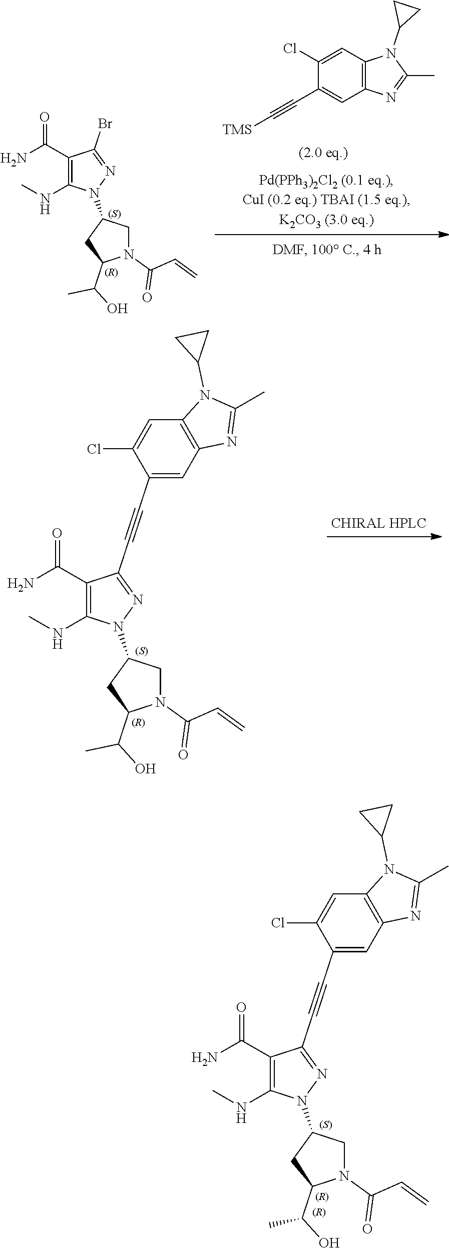

1-((3S,5R)-1-Acryloyl-5-((R)-1-hydroxyethyl)pyrrolidin-3-yl)-3-((6-chloro-1-cyclopropyl-2-methyl-1H-benzo[d]imidazol-5-yl)ethynyl)-5-(methylamino)-1H-pyrazole-4-carboxamide. MS ESI calculated for C₂₇H₃₀ClN₇O₃[M+H]⁺, 536.21, found 536.35; ¹H NMR (400 MHz, DMSO-d₆) δ 7.86 (s, 1H), 7.71 (s, 1H), 7.53 (s, 1H), 6.94-6.78 (m, 1H), 6.77-6.53 (m, 2H), 6.20-6.08 (m, 1H), 5.73-5.65 (m, 1H), 5.44-5.28 (m, 1H), 5.06-5.00 (m, 1H), 4.36-3.66 (m, 3H), 3.33 (s, 2H), 2.95 (m, 3H), 2.60 (s, 3H), 2.54-2.18 (m, 2H), 1.28-1.16 (m, 2H), 1.09-1.04 (m, 5H).

### Example 221: 1-((3S,5R)-1-Acryloyl-5-((S)-1-hydroxyethyl)pyrrolidin-3-yl)-3-((6-chloro-1-cyclopropyl-2-methyl-1H-benzo[d]imidazol-5-yl)ethynyl)-5-(methylamino)-1H-pyrazole-4-carboxamide

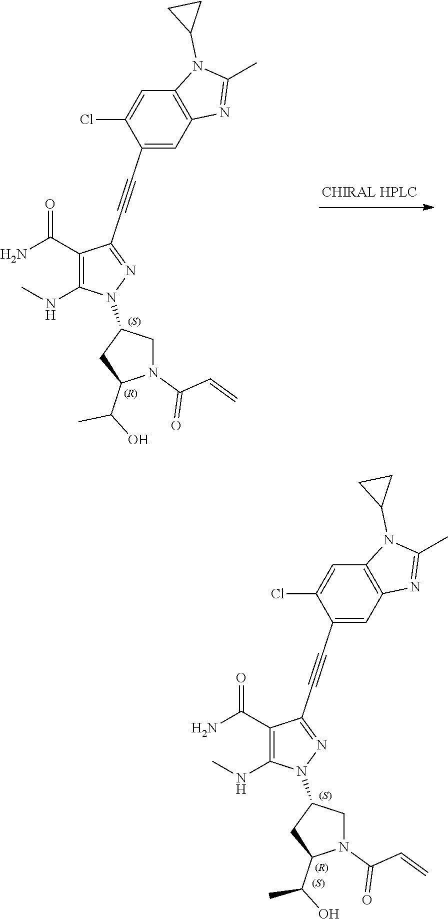

The racemic product (0.30 g) was purified by Prep-SFC with the following conditions: Column: CHIRAL ART Cellulose-SC, 2×25 cm, 5 μm; Mobile Phase A: Hex:DCM=1:1 (0.5% 2M NH₃-MeOH), Mobile Phase B: EtOH; Flow rate: 20 mL/min; Gradient: 50% B to 50% B in 12 min; Wave length: 220/254 nm; RT2: 10.04 min; Sample solvent: EtOH:DCM=1:1; Injection Volume: 0.3 mL; RT2: 10.04 min. The fractions contained desired product were combined and concentrated to afford 1-((3S,5R)-1-acryloyl-5-((S)-1-hydroxyethyl)pyrrolidin-3-yl)-3-((6-chloro-1-cyclopropyl-2-methyl-1H-benzo[d]imidazol-5-yl)ethynyl)-5-(methylamino)-1H-pyrazole-4-carboxamide (0.12 g, 35%) as a white solid. MS ESI calculated for C₂₇H₃₀ClN₇O₃ [M+H]⁺, 536.21, found 536.45; ¹H NMR (400 MHz, DMSO-4) S 7.85 (s, 1H), 7.71 (s, 1H), 7.51 (s, 1H), 6.99-6.50 (m, 3H), 6.20-6.13 (m, 1H), 5.70-5.62 (m, 1H), 5.31-5.19 (m, 1H), 5.03-4.95 (m, 1H), 4.50-4.20 (m, 1H), 4.06-3.95 (m, 2H), 3.75-3.66 (m, 1H), 3.40-3.34 (m, 1H), 3.02-2.95 (m, 3H), 2.60 (s, 3H), 2.37-2.30 (m, 2H), 1.25-1.18 (m, 2H), 1.11-0.99 (m, 5H).

### Example 222: 3-[2-(6-Chloro-1-cyclopropyl-1,3-benzodiazol-5-yl)ethynyl]-1-[(3S,5R)-5-[(1R)-1-hydroxyethyl]-1-(prop-2-enoyl)pyrrolidin-3-yl]-5-(methylamino)pyrazole-4-carboxamide

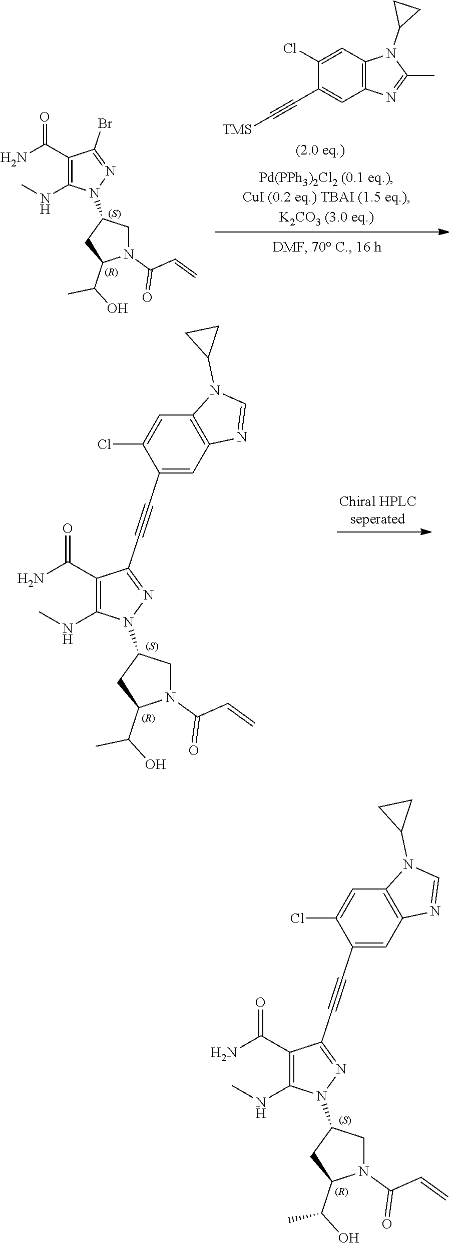

3-[2-(6-chloro-1-cyclopropyl-1,3-benzodiazol-5-yl)ethynyl]-1-[(3S,5R)-5-[(1R)-1-hydroxyethyl]-1-(prop-2-enoyl)pyrrolidin-3-yl]-5-(methylamino)pyrazole-4-carboxamide. MS ESI calculated for C₂₆H₂₈ClN₇O₃[M+H]⁺, 522.19, found 522.15; ¹H NMR (400 MHz, CDCl₃) δ 8.39 (s, 1H), 8.02 (s, 1H), 7.88 (s, 1H), 7.49 (s, 1H), 6.84 (s, 1H), 6.72-6.57 (m, 2H), 6.21-6.13 (m, 1H), 5.71-5.66 (m, 1H), 5.39-5.32 (m, 1H), 5.09-4.89 (m, 1H), 4.27-4.16 (m, 1H), 3.92-3.76 (m, 2H), 3.57-3.52 (m, 1H), 2.96 (t, J=12 Hz, 3H), 2.39-2.09 (m, 3H), 1.15-1.04 (m, 7H).

### Example 223: 3-[2-(6-Chloro-1-cyclopropyl-1,3-benzodiazol-5-yl)ethynyl]-1-[(3S,5R)-5-[(1S)-1-hydroxyethyl]-1-(prop-2-enoyl)pyrrolidin-3-yl]-5-(methylamino)pyrazole-4-carboxamide

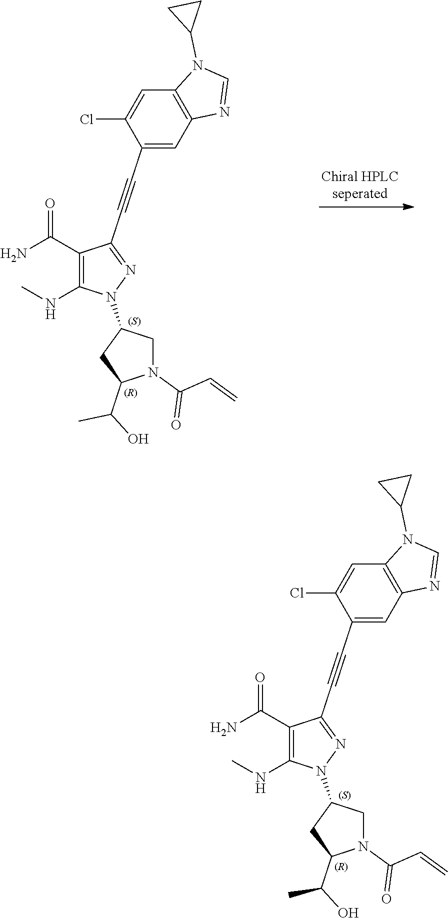

3-[2-(6-chloro-1-cyclopropyl-1,3-benzodiazol-5-yl)ethynyl]-1-[(3S,5R)-5-[(1S)-1-hydroxyethyl]-1-(prop-2-enoyl)pyrrolidin-3-yl]-5-(methylamino)pyrazole-4-carboxamide. MS ESI calculated for C₂₆H₂₈ClN₇O₃[M+H]⁺, 522.19, found 522.20; ¹H NMR (400 MHz, CDCl₃) δ 8.41 (s, 1H), 8.03 (s, 1H), 7.89 (s, 1H), 7.52 (s, 1H), 6.87-6.57 (m, 3H), 6.15 (d, J=10 Hz, 1H), 5.71-5.64 (m, 1H), 5.28 (s, 1H), 5.04-4.97 (m, 1H), 4.43-4.30 (m, 1H), 4.05-3.97 (m, 2H), 3.74-3.68 (m, 1H), 3.55 (s, 1H), 2.97-2.95 (m, 3H), 2.51 (s, 1H), 2.35 (s, 1H), 1.13-1.01 (m, 7H).

### Example 224: 3-[2-(1-Cyclopropyl-6-fluoro-2-methyl-1,3-benzodiazol-5-yl)ethynyl]-1-[(3S,5R)-5-[(1R)-1-hydroxyethyl]-1-(prop-2-enoyl)pyrrolidin-3-yl]-5-(methylamino)pyrazole-4-carboxamide

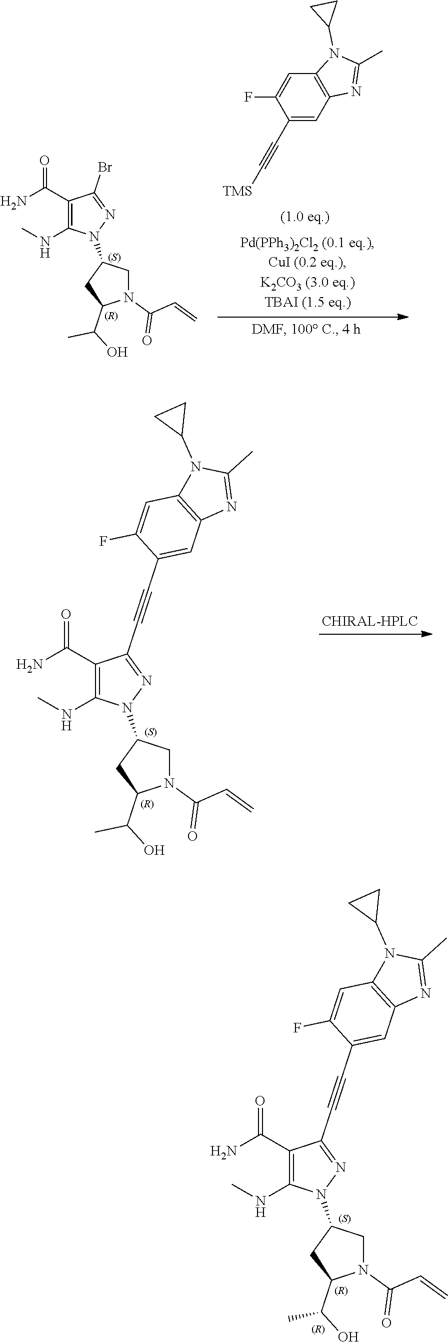

3-[2-(1-cyclopropyl-6-fluoro-2-methyl-1,3-benzodiazol-5-yl)ethynyl]-1-[(3S,5R)-5-[(1R)-1-hydroxyethyl]-1-(prop-2-enoyl)pyrrolidin-3-yl]-5-(methylamino)pyrazole-4-carboxamide. MS ESI calculated for C₂₇H₃₀FN₇O₃. [M+H]⁺, 520.24, found 520.25; ¹H NMR (400 MHz, DMSO-d₆) δ 7.79 (d, J=6.4 Hz, 1H), 7.50 (d, J=9.6 Hz, 2H), 6.78-6.54 (m, 3H), 6.23-6.11 (m, 1H), 5.73-5.61 (m, 1H), 5.41-5.29 (m, 1H), 5.06-4.89 (m, 1H), 4.33-3.70 (m, 4H), 2.95 (t, J=5.7 Hz, 3H), 2.59 (s, 3H), 2.46 (s, 3H), 1.25-1.11 (m, 2H), 1.11-1.02 (m, 5H).

### Example 225: 3-[2-(1-Cyclopropyl-6-fluoro-2-methyl-1,3-benzodiazol-5-yl)ethynyl]-1-[(3S,5R)-5-[(1S)-1-hydroxyethyl]-1-(prop-2-enoyl)pyrrolidin-3-yl]-5-(methylamino)pyrazole-4-carboxamide

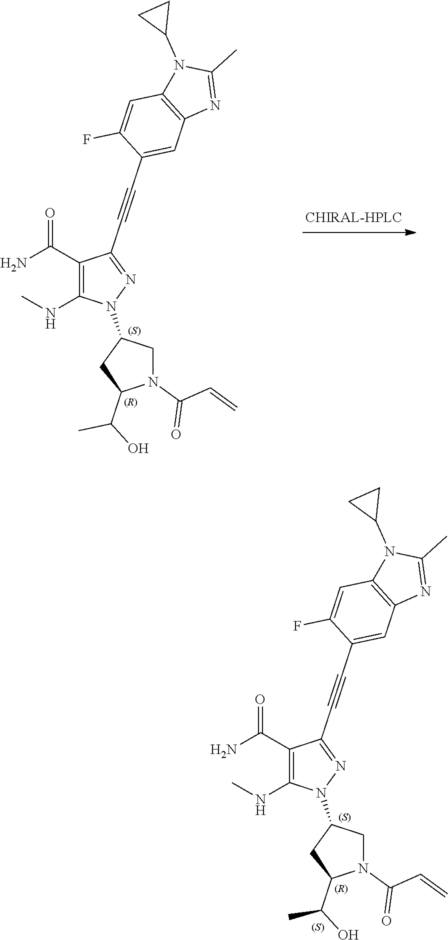

3-[2-(1-cyclopropyl-6-fluoro-2-methyl-1,3-benzodiazol-5-yl)ethynyl]-1-[(3S,5R)-5-[(1S)-1-hydroxyethyl]-1-(prop-2-enoyl)pyrrolidin-3-yl]-5-(methylamino)pyrazole-4-carboxamide. MS ESI calculated for C₂₇H₃₀FN₇O₃[M+H]⁺, 520.24, found 520.60; ¹H NMR (400 MHz, DMSO-d₆) δ 7.78 (d, J=6.3 Hz, 1H), 7.49 (d, J=9.7 Hz, 2H), 6.83-6.54 (m, 3H), 6.15 (d, J=17.0 Hz, 1H), 5.72-5.61 (m, 1H), 5.31-5.20 (m, 1H), 5.01-4.94 (m, 1H), 4.48-4.24 (m, 1H), 4.06-3.90 (m, 2H), 3.79-3.68 (m, 1H), 3.01-2.89 (m, 3H), 2.59 (s, 3H), 2.49 (s, 1H), 2.40-2.29 (m, 1H), 1.25-1.12 (m, 2H), 1.12-1.05 (m, 2H), 1.03 (s, 3H).

### Example 226: 3-[2-(6-Chloro-1-cyclopropyl-1,3-benzodiazol-5-yl)ethynyl]-1-[(3S,5R)-1-(4-hydroxy-methylpent-2-ynoyl)-5-(methoxymethyl)pyrrolidin-3-yl]-5-(methylamino)pyrazole-4-carboxamide

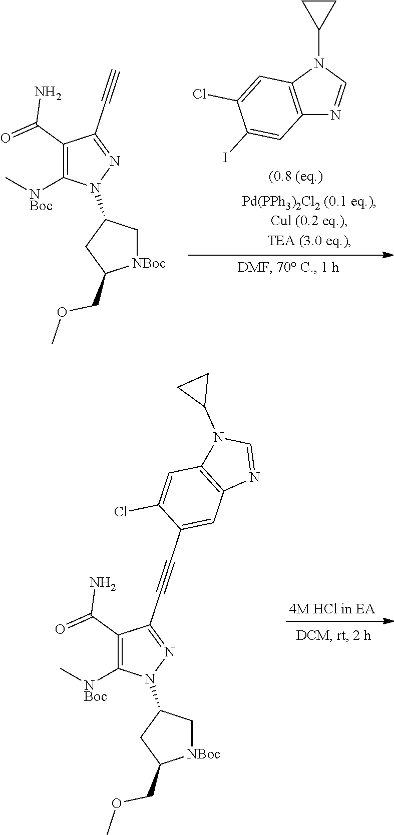 [SMILES: C#Cc1nn([C@H]2C[C@H](COC)N(C(=O)OC(C)(C)C)C2)c(N(C)C(=O)OC(C)(C)C)c1C(N)=O ; Clc1cc2c(cc1I)ncn2C1CC1 ; COC[C@H]1C[C@H](n2nc(C#Cc3cc4ncn(C5CC5)c4cc3Cl)c(C(N)=O)c2N(C)C(=O)OC(C)(C)C)CN1C(=O)OC(C)(C)C]

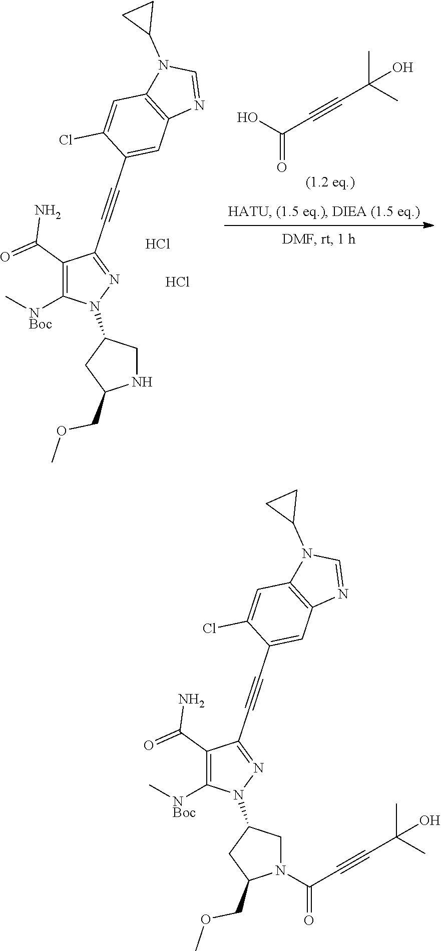

3-[2-(6-chloro-1-cyclopropyl-1,3-benzodiazol-5-yl)ethynyl]-1-[(3S,5R)-1-(4-hydroxy-4-methylpent-2-ynoyl)-5-(methoxymethyl)pyrrolidin-3-yl]-5-(methylamino)pyrazole-4 carboxamide. MS ESI calculated for C₂₉H₃₂ClN₇O₄ [M+H]+, 578.22, found 578.30; ¹H NMR (400 MHz, Chloroformn-d) δ 8.17-8.06 (m, 2H), 7.69 (s, 1H), 7.19 (s, 1H), 6.84 (s, 1H), 5.56-5.29 (m, 2H), 4.48 (d, J=8.7 Hz, 1H), 4.18 (d, J=10.6, 8.1 Hz, 1H), 4.06 (d, J=17.8, 7.3 Hz, 1H), 3.94-3.85 (m, 1H), 3.79-3.53 (m, 1H), 3.47-3.36 (m, 3H), 3.04 (d, J=14.9 Hz, 3H), 2.86-2.74 (m, 1H), 2.17 (s, 2H), 1.60 (d, J=13.1 Hz, 6H), 1.30-1.21 (m, 2H), 1.15-1.07 (in, 2H).

### Example 227: 3-[2-(1-Cyclopropyl-6-fluoro-2-methyl-1,3-benzodiazol-5-yl)ethynyl]-1-[(3 S,5R)-1-(4-hydroxy-4-methylpent-2-ynoyl)-5-(methoxymethyl)pyrrolidin-3-yl]-5-(methylamino)pyrazole-4-carboxamide

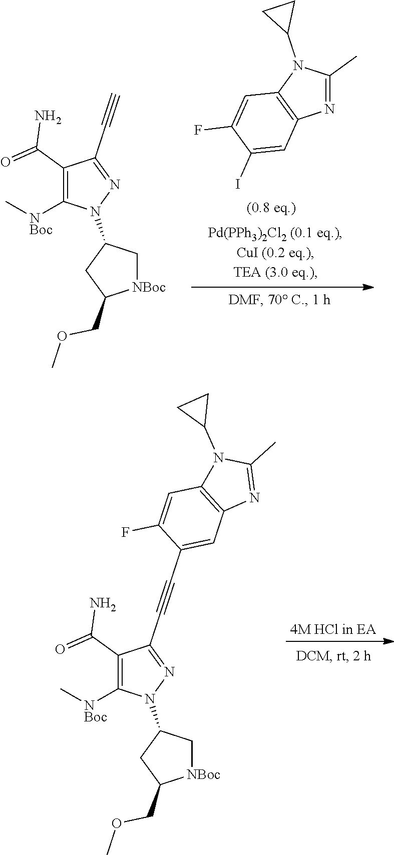

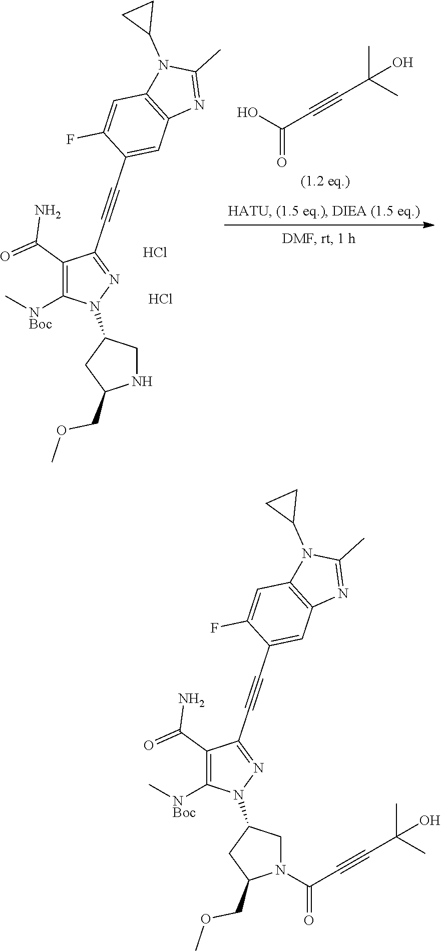

3-[2-(1-cyclopropyl-6-fluoro-2-methyl-1,3-benzodiazol-5-yl)ethynyl]-1-[(3S,5R)-1-(4-hydroxy-4-methylpent-2-ynoyl)-5-(methoxymethyl)pyrrolidin-3-yl]-5-(methylamino)pyrzole-4-carboxamide. MS ESI calculated for C₃₀H₃₄FN₇O₄[M+H]+, 576.27, found 576.30; ¹H NMR (400 MHz, Chloroform-d) δ 7.82 (t, J=7.4 Hz, 1H), 7.13 (s, 1H), 6.82 (d, J=5.9 Hz, 1H), 5.50-5.30 (m, 2H), 4.47 (di, J=8.8 Hz, 1H), 4.21-4.12 (m, 1H), 4.12-4.01 (m, 1H), 3.92-3.83 (m, 1H), 3.73-3.59 (m, 1H), 3.40 (d, J=13.0 Hz, 3H), 3.29 (s, 1H), 3.09-2.96 (m, 3H), 2.75 (di, J=4.2 Hz, 3H), 2.43-2.28 (m, 2H), 1.60 (di, J=14.8 Hz, 6H), 1.35-1.25 (m, 2H), 1.10 (s, 2H).

### Example 228: 3-[2-(6-Chloro-1-cyclopropyl-2-methyl-1,3-benzodiazol-5-yl)ethynyl]-1-[(3 S,5R)-1-(4-hydroxy-4-methylpent-2-ynoyl)-5-(methoxymethyl)pyrrolidin-3-yl]-5-(methylamino)pyrazole-4-carboxamide

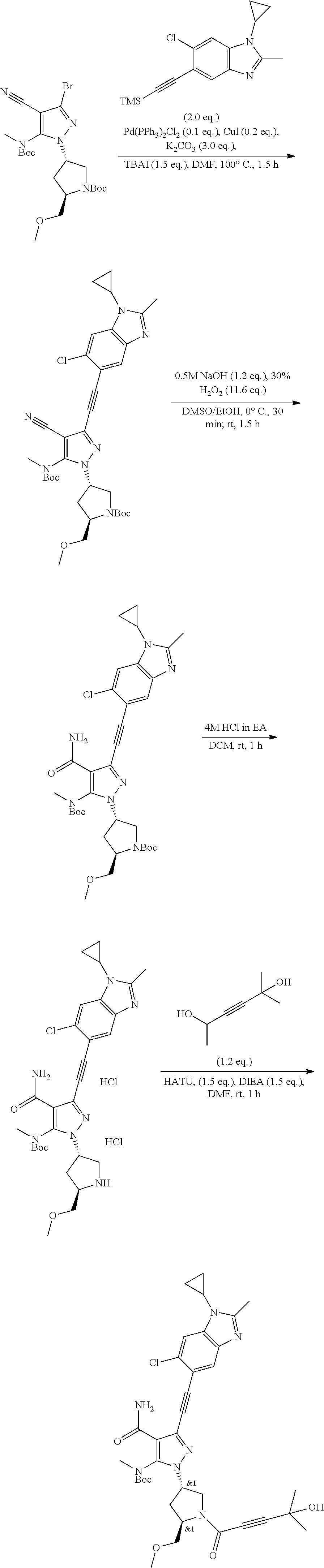

3-[2-(6-chloro-1-cyclopropyl-2-methyl-1,3-benzodiazol-5-yl)ethynyl]-1-[(3S,5R)-1-(4-hydroxy-4-methylpent-2-ynoyl)-5-(methoxymethyl)pyrrolidin-3-yl]-5-(methylamino)pyrazole-4-carboxamide. MS ESI calculated for C₃₀H₃₄ClN₇O₄[M+H]⁺, 592.24, found 592.30; ¹H NMR (400 MHz, CDCl₃) δ 7.93 (s, 1H), 7.57 (s, 1H), 7.28 (s, 1H), 6.83 (s, 1H), 5.48-5.35 (m, 2H), 4.52-4.43 (m, 1H), 4.18-4.13 (m, 2H), 3.77-3.74 (m, 1H), 3.70-3.54 (m, 1H), 3.42-3.38 (m, 1H), 3.23 (s, 2H), 3.06-3.01 (m, 1H), 2.69 (s, 4H), 2.46 (s, 3H), 2.40-2.30 (m, 2H), 1.61-1.58 (m, 6H), 1.31-1.29 (m, 2H), 1.08 (s, 2H).

### Example 230: 1-((3S,5R)-1-Acryloyl-5-(methoxymethyl)pyrrolidin-3-yl)-3-((1-cyclobutyl-6-fluoro-1H-benzo[d]imidazol-5-yl)ethynyl)-5-(methylamino)-1H-pyrazole-4-carboxamide

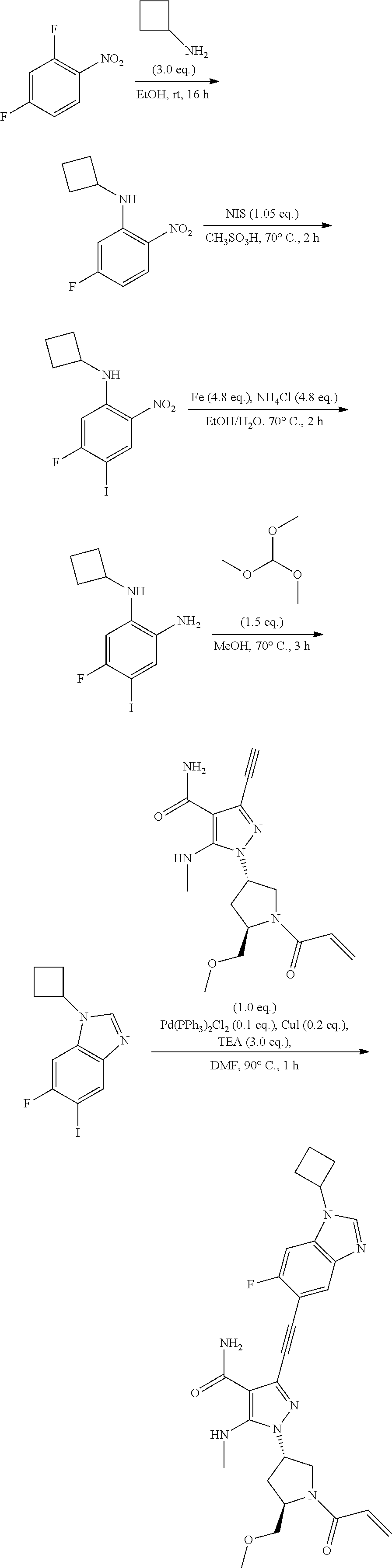

1-((3S,5R)-1-Acryloyl-5-(methoxymethyl)pyrrolidin-3-yl)-3-((1-cyclobutyl-6-fluoro-1H-benzo[d]imidazol-5-yl)ethynyl)-5-(methylamino)-1H-pyrazole-4-carboxamide. MS ESI calculated for C₂₇H₃₀FN₇O₃[M+H]⁺, 520.24, found 520.25; ¹H NMR (400 MHz, CDCl₃) δ 8.13 (d, J=25.1 Hz, 1H), 8.00 (d, J=5.9 Hz, 1H), 7.17 (d, J=9.4 Hz, 2H), 6.81 (s, 1H), 6.61-6.32 (m, 2H), 5.75-5.68 (m, 1H), 5.61 (s, 1H), 5.57-5.22 (m, 1H), 4.85-6.78 (m, 1H), 4.64-4.37 (m, 1H), 4.15-4.09 (m, 1H), 4.06-4.00 (m, 1H), 3.94-3.87 (m, 1H), 3.56-3.42 (m, 1H), 3.38 (di, J=4.4 Hz, 3H), 3.04 (di, J=15.5 Hz, 3H), 2.79-2.62 (m, 3H), 2.58-2.50 (m, 2H), 2.38-2.30 (m, 1H), 2.09-2.01 (m, 2H).

### Example 231: 1-((3S,5R)-1-Acryloyl-5-(methoxymethyl)pyrrolidin-3-yl)-3-((1-cyclobutyl-6-fluoro-2-methyl-1H-benzo[d]imidazol-5-yl)ethynyl)-5-(methylamino)-1H-pyrazole-4-carboxamide

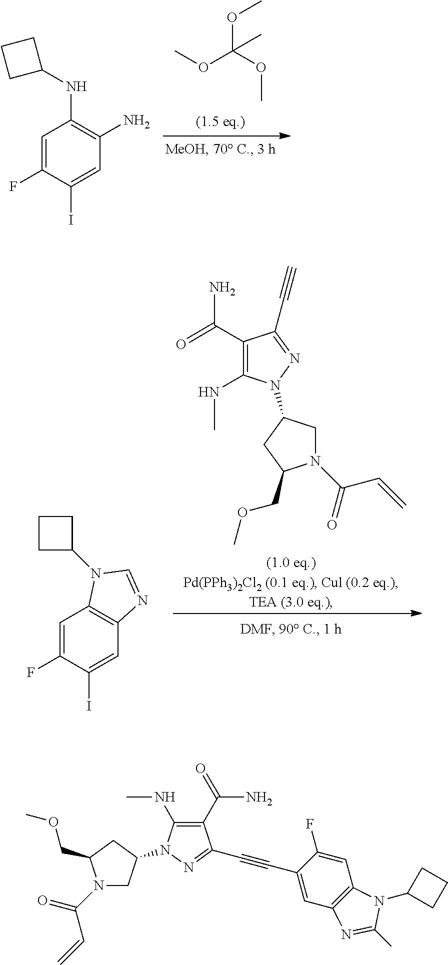

1-((3S,5R)-1-acryloyl-5-(methoxymethyl)pyrrolidin-3-yl)-3-((1-cyclobutyl-6-fluoro-2-methyl-1H-benzo[d]imidazol-5-yl)ethynyl)-5-(methylamino)-1H-pyrazole-4-carboxamide. MS ESI calculated for C₂₈H₃₂FN₇O₃ [M+H]⁺, 534.26, found 534.40; ¹H NMR (400 MHz, CDCl₃) δ 7.88 (d, J=6.2 Hz, 1H), 7.34 (d, J=10.3 Hz, 1H), 7.13 (s, 1H), 6.84 (d, J=6.3 Hz, 1H), 6.44 (d, J=7.9 Hz, 2H), 5.78-5.67 (m, 1H), 5.59-5.46 (m, 1H), 5.33 (d, J=26.1 Hz, 1H), 4.89-4.82 (m, 1H), 4.57 (d, J=8.9 Hz, 1H), 4.15-4.10 (m, 1H), 4.06-4.00 (m, 1H), 3.92 (d, J=9.8 Hz, 1H), 3.57-3.43 (m, 1H), 3.39 (d, J=3.9 Hz, 3H), 3.13-2.98 (m, 3H), 2.92-2.70 (m, 3H), 2.65 (s, 5H), 2.44-2.25 (m, 1H), 2.09-2.01 (m, 2H).

### Example 232: 3-[2-(6-Chloro-1-cyclobutyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

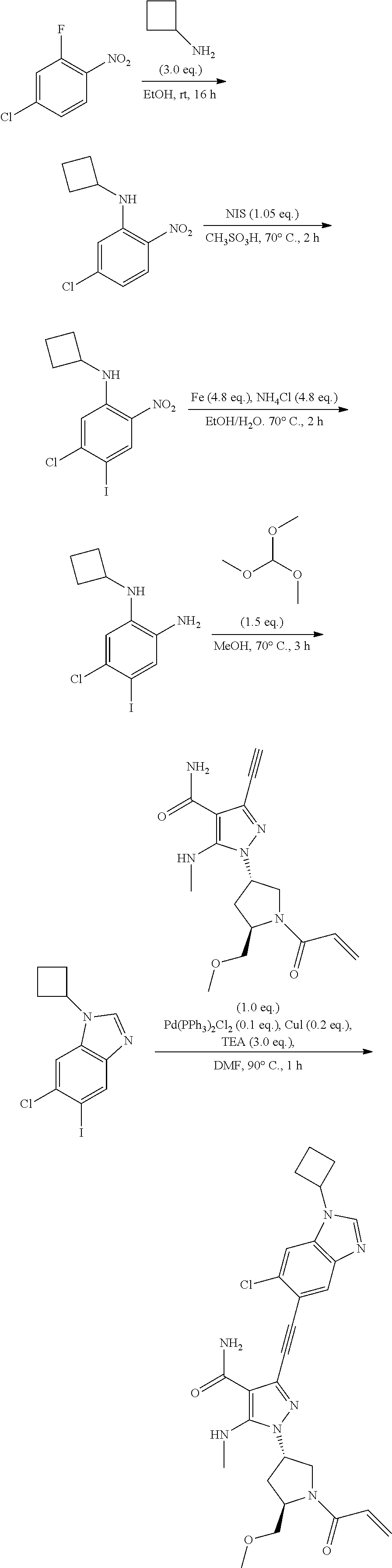

3-[2-(6-chloro-1-cyclobutyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide. MS ESI calculated for C₂₇H₃₀ClN₇O₃[M+H]⁺, 536.21, found 536.15; ¹H NMR (400 MHz, CDCl₃) δ 8.10 (s, 2H), 7.72-7.67 (m, 1H), 7.21 (s, 1H), 6.83 (s, 1H), 6.57-6.39 (m, 2H), 5.73-5.70 (m, 1H), 5.56-5.48 (m, 1H), 5.36-5.28 (m, 1H), 4.85-4.77 (m, 1H), 4.57 (d, J=12 Hz, 1H), 4.15-3.96 (m, 2H), 3.93-3.90 (m, 1H), 3.52-3.38 (m, 4H), 3.07-2.97 (m, 3H), 2.77-2.66 (m, 3H), 2.61-2.51 (m, 2H), 2.41-2.29 (m, 1H), 2.11-2.02 (m, 2H).

### Example 233: 1-((3S,5R)-1-Acryloyl-5-(methoxymethyl)pyrrolidin-3-yl)-3-((6-chloro-1-cyclobutyl-2-methyl-1H-benzo[d]imidazol-5-yl)ethynyl)-5-(methylamino)-1H-pyrazole-4-carboxamide

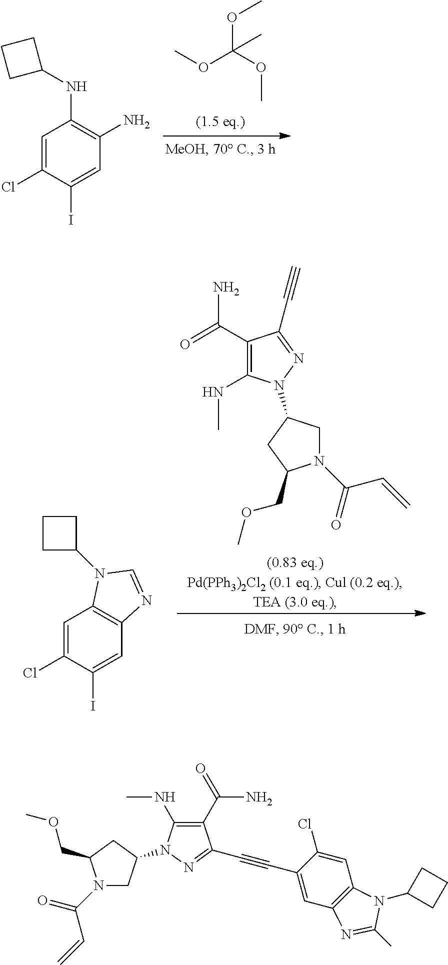

1-((3S,5R)-1-acryloyl-5-(methoxymethyl)pyrrolidin-3-yl)-3-((6-chloro-1-cyclobutyl-2-methyl-1H-benzo[d]imidazol-5-yl)ethynyl)-5-(methylamino)-1H-pyrazole-4-carboxamide. MS ESI calculated for C₂₈H₃₂ClN₇O₃ [M+H]⁺, 550.23, found 550.30; ¹H NMR (400 MHz, Methanol-d₄) δ 7.93-7.90 (s, 1H), 7.86-7.70 (m, 1H), 6.71-6.70 (m, 1H), 6.32-6.20 (m, 1H), 5.78-5.70 (m, 1H), 5.44-5.30 (m, 1H), 5.07-5.01 (m, 1H), 4.69-4.52 (m, 1H), 4.16-4.04 (m, 2H), 3.95-3.78 (m, 1H), 3.56-3.50 (m, 1H), 3.41-3.40 (m, 3H), 3.04-3.01 (m, 3H), 2.84-2.80 (m, 2H), 2.75-2.57 (m, 6H), 2.46-2.40 (m, 1H), 2.15-1.98 (m, 2H).

### Example 234: 3-{2-[6-Fluoro-1-(oxetan-3-yl)-1,3-benzodiazol-5-yl]ethynyl}-1-[(3S,5R)-5-(methoxymethyl)-1-(prop-2-enoyl)pyrrolidin-3-yl]-5-(methylamino)pyrazole-4-carboxamide

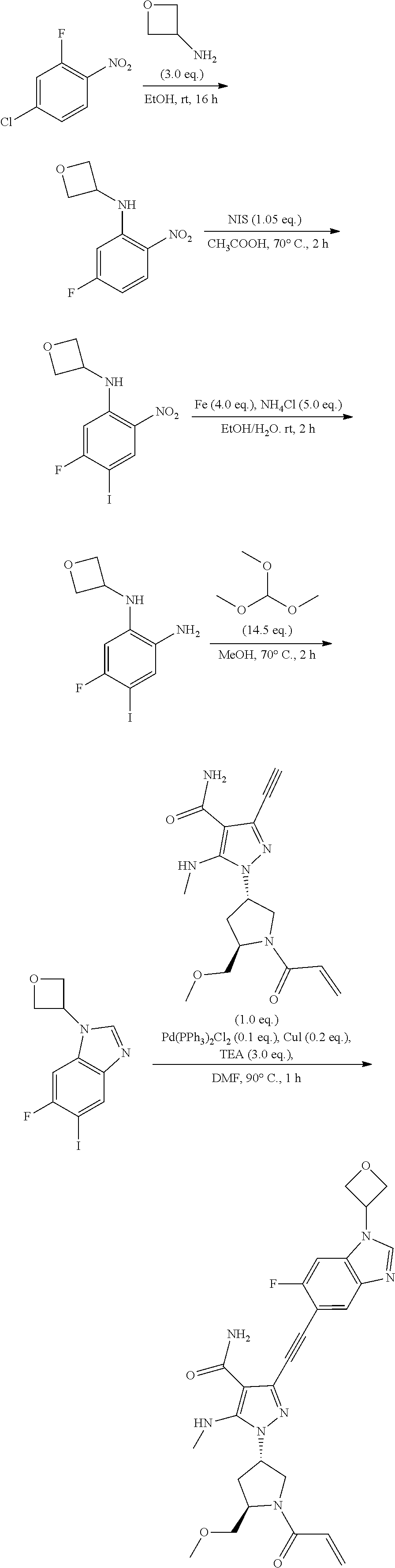

3-12-[6-fluoro-1-(oxetan-3-yl)-1,3-benzodiazol-5-yl]ethynyl)-1-[(3S,5R)-5-(methoxymethyl)-1-(prop-2-enoyl)pyrrolidin-3-yl]-5-(methylamino)pyrazole-4-carboxamide. MS ESI calculated for C₂₆H₂₈FN₇O₄[M+H]⁺, 522.22, found 522.25; ¹H NMR (400 MHz, CDCl₃) δ 8.13 (s, 1H), 8.04 (s, 1H), 7.57 (d, J=8.40 Hz, 1H), 7.10 (s, 1H), 6.83 (s, 1H), 6.55-6.35 (m, 2H), 5.74-5.70 (m, 1H), 5.54-5.50 (m, 2H), 5.48-5.26 (m, 3H), 5.10-5.07 (m, 2H), 4.56 (d, J=8.40 Hz, 1H), 4.14-4.10 (m, 1H), 4.09-4.02 (m, 1H), 3.93-3.90 (m, 1H), 3.53-3.40 (m, 1H), 3.37 (d, J=4.40 Hz, 3H), 3.04 (d, J=14.40 Hz, 3H), 2.70-2.68 (m, 1H), 2.42-2.25 (m, 1H).

### Example 235: 3-{2-[6-Fluoro-2-methyl-1-(oxetan-3-yl)-1,3-benzodiazol-5-yl]ethynyl}-1-[(3 S,5R)-5-(methoxymethyl)-1-(prop-2-enoyl)pyrrolidin-3-yl]-5-(methylamino)pyrazole-4-carboxamide

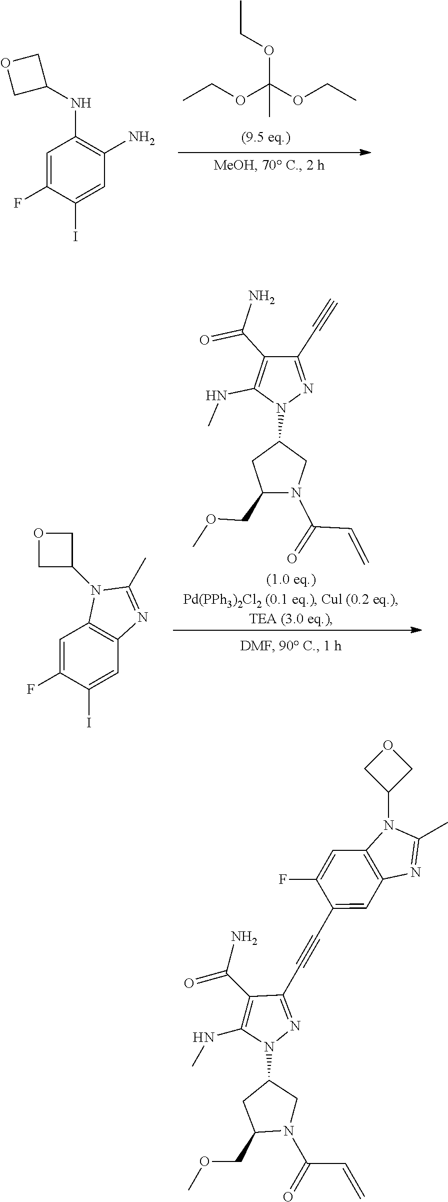

3-{2-[6-fluoro-2-methyl-1-(oxetan-3-yl)-1,3-benzodiazol-5-yl]ethynyl)-1-[(3S,5R)-5-(methoxymethyl)-1-(prop-2-enoyl)pyrrolidin-3-yl]-5-(methylamino)pyrazole-4-carboxamide. MS ESI calculated for C₂₇H₃₀FN₇O₄[M+H]⁺, 536.23, found 536.65; ¹H NMR (400 MHz, CDCl₃) δ 7.93-7.88 (m, 2H), 7.09 (s, 1H), 6.83 (s, 1H), 6.58-6.33 (m, 2H), 5.75-5.70 (m, 1H), 5.54-5.50 (m, 2H), 5.31-5.27 (m, 3H), 5.19-5.11 (m, 2H), 4.60-4.37 (m, 1H), 4.15-4.10 (m, 1H), 4.06-4.02 (m, 1H), 3.94-3.90 (m, 1H), 3.53-3.41 (m, 1H), 3.40-3.38 (m, 3H), 3.08-2.95 (m, 3H), 2.78-2.57 (m, 4H), 2.43-2.26 (m, 1H).

### Example 236: 3-[2-(6-Fluoro-3-methylquinolin-7-yl)ethynyl]-1-[(3S,5R)-5-(methoxymethyl)-1-(prop-2-enoyl)pyrrolidin-3-yl]-5-(methylamino)pyrazole-4-carboxamide

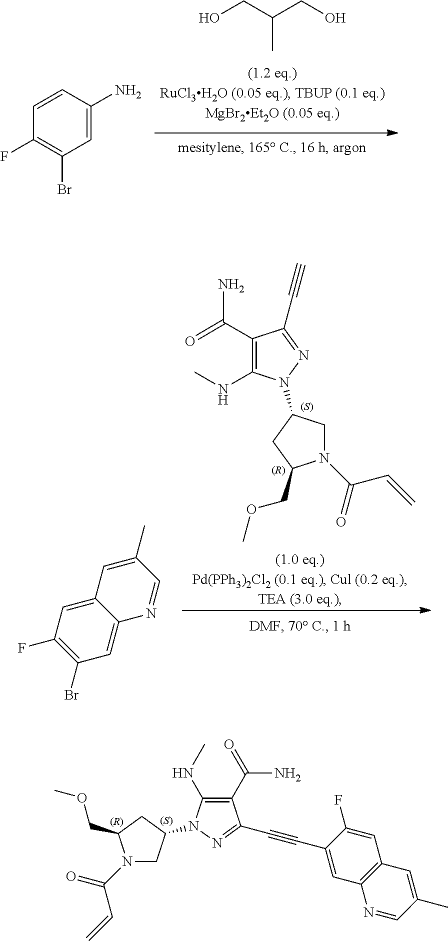

3-[2-(6-fluoro-3-methylquinolin-7-yl)ethynyl]-1-[(3S,5R)-5-(methoxymethyl)-1-(prop-2-enoyl)pyrrolidin-3-yl]-5-(methylamino)pyrazole-4-carboxamide. MS ESI calculated for C₂₆H₂₇FN₆O₃[M+H]⁺, 491.21, found 491.30; ¹H NMR (400 MHz, Chloroform-d) δ 8.78 (s, 1H), 8.36 (d, J=6.7 Hz, 1H), 7.90 (s, 1H), 7.43 (d, J=9.5 Hz, 1H), 7.16-6.98 (m, 1H), 6.84 (s, 1H), 6.56-6.36 (m, 2H), 5.71-5.68 (m, 1H), 5.58-5.45 (m, 1H), 5.41 (s, 1H), 4.56 (d, J=9.0 Hz, 1H), 4.11 (t, J=9.1 Hz, 1H), 4.09-3.96 (m, 1H), 4.00-3.87 (m, 1H), 3.58-3.42 (m, 1H), 3.37 (d, J=4.8 Hz, 3H), 3.08-3.00 (m, 3H), 2.77-2.65 (m, 1H), 2.55 (s, 3H), 2.44-2.27 (m, 1H).

### Example 237: 1-((3S,5R)-1-Acryloyl-5-(methoxymethyl)pyrrolidin-3-yl)-3-((6-fluoro-1-isopropyl-1H-benzo[d]imidazol-5-yl)ethynyl)-5-(methylamino)-1H-pyrazole-4-carboxamide

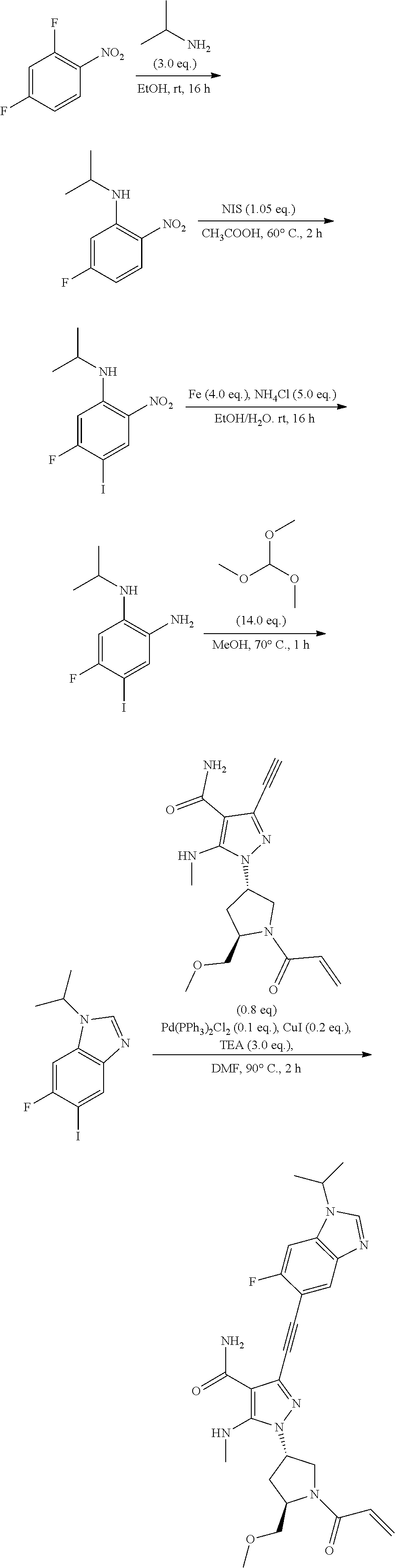

1-((3S,5R)-1-Acryloyl-5-(methoxymethyl)pyrrolidin-3-yl)-3-((6-fluoro-1-isopropyl-1H-benzo[d]imidazol-5-yl)ethynyl)-5-(methylamino)-1H-pyrazole-4-carboxamide. MS ESI calculated for C₂₆H₃₀FN₇O₃[M+H]⁺, 508.60, found 508.40; ¹H NMR (400 MHz, CDCl₃) δ 8.09 (d, J=42.0 Hz, 2H), 7.18 (d, J=40.0 Hz, 2H), 6.78 (d, J=46.0 Hz, 1H), 6.54-6.39 (m, 2H), 5.72 (dd, J=8.5, 3.8 Hz, 1H), 5.53-5.24 (m, 2H), 4.60 (dd, J=16.4, 8.4 Hz, 2H), 4.18-4.09 (m, 2H), 3.92 (dd, J=9.7, 2.9 Hz, 1H), 3.53-3.37 (m, 4H), 3.05 (d, J=15.5 Hz, 3H), 2.78-2.66 (m, 1H), 2.38-2.34 (m, 1H), 1.66 (d, J=6.7 Hz, 6H).

### Example 238: 3-{2-[6-Fluoro-1-(1-methylcyclopropyl)-1,3-benzodiazol-5-yl]ethynyl}-1-[(3S,5R)-5-(methoxymethyl)-1-(prop-2-enoyl)pyrrolidin-3-yl]-5-(methylamino)pyrazole-4-carboxamide

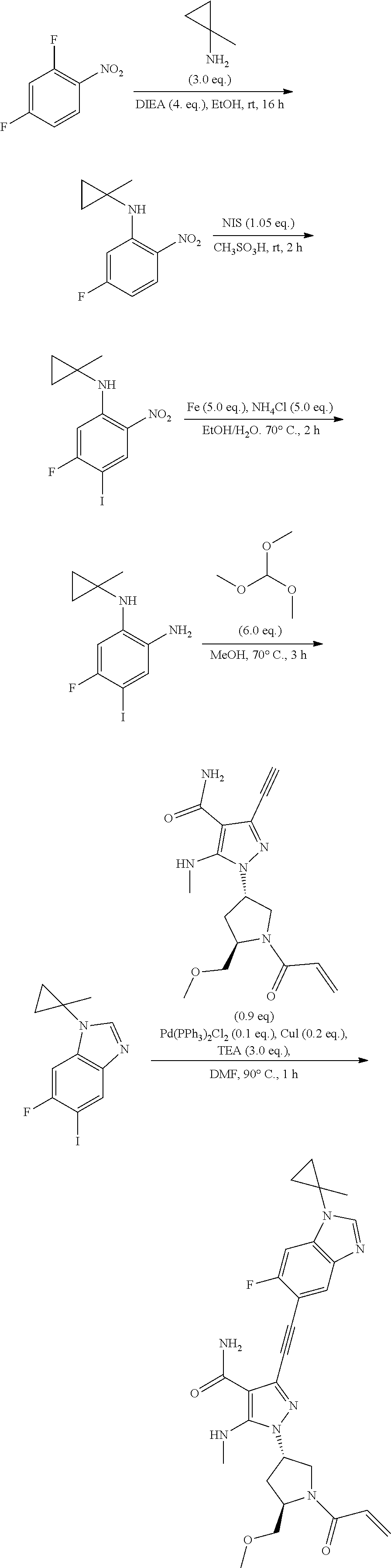

3-{2-[6-fluoro-1-(1-methylcyclopropyl)-1,3-benzodiazol-5-yl]ethynyl}-1-[(3S,5R)-5-(methoxymethyl)-1-(prop-2-enoyl)pyrrolidin-3-yl]-5-(methylamino)pyrazole-4-carboxamide. MS ESI calculated for C₂₇H₃₀FN₇O₃[M+H]⁺, 520.24, found 520.35; ¹H NMR (400 MHz, DMSO-d₆) δ 8.43-8.41 (s, 1H), 7.96-7.95 (m, 1H), 7.74-7.59 (m, 1H), 7.52-7.50 (m, 1H), 6.87-6.53 (m, 3H), 6.17-6.16 (m, 1H), 5.69-5.67 (m, 1H), 5.28-5.20 (m, 1H), 4.59-4.35 (m, 1H), 4.06-3.74 (m, 2H), 3.68-3.43 (m, 2H), 3.31-3.30 (m, 3H), 2.96-2.94 (m, 3H), 2.63-2.62 (m, 1H), 2.30-2.28 (m, 1H), 1.55-1.52 (s, 3H), 1.19-1.17 (m, 2H), 1.11-1.00 (m, 2H).

### Example 239: 1-((3S,5R)-1-Acryloyl-5-(methoxymethyl)pyrrolidin-3-yl)-3-((1-(1-cyanocyclopropyl)-6-fluoro-1H-benzo[d]imidazol-5-yl)ethynyl)-5-(methylamino)-1H-pyrazole-4-carboxamide

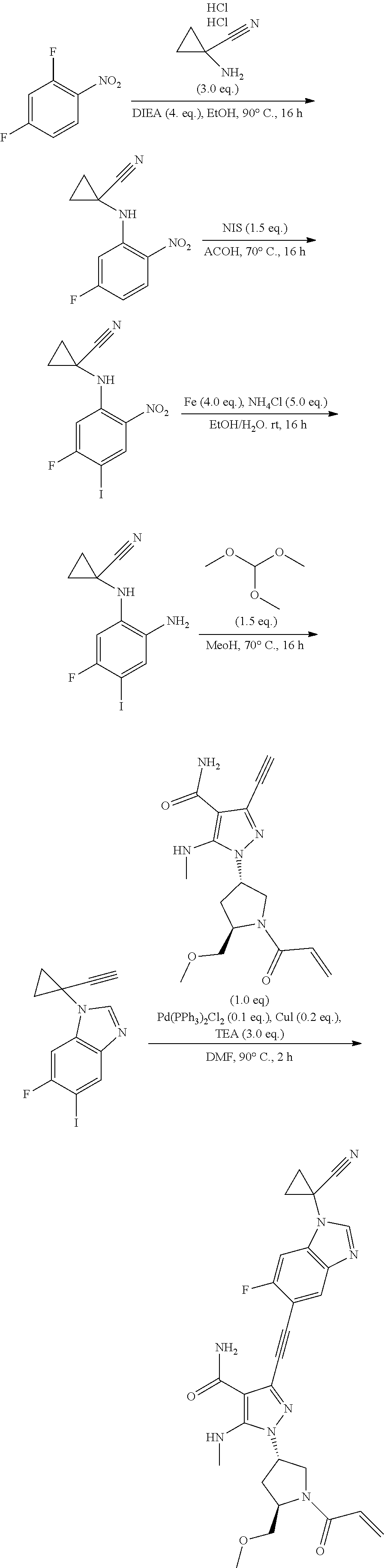

1-((3S,5R)-1-acryloyl-5-(methoxymethyl)pyrrolidin-3-yl)-3-((1-(1-cyanocyclopropyl)-6-fluoro-1H-benzo[d]imidazol-5-yl)ethynyl)-5-(methylamino)-1H-pyrazole-4-carboxamide. MS ESI calculated for C₂₇H₂₇FN₈O₃[M+H]+, 531.22, found 531.30; ¹H NMR (400 MHz, Chloroform-d) δ 8.02 (s, 2H), 7.37 (d, J=8.6 Hz, 1H), 7.02 (d, J=16.9 Hz, 1H), 6.76 (dd, J=45.9, 6.1 Hz, 1H), 6.57-6.35 (m, 2H), 5.70 (dd, J=8.4, 3.9 Hz, 1H), 5.60-5.14 (m, 2H), 4.48 (d, J=54.9 Hz, 1H), 4.12-3.99 (m, 2H), 3.90 (dd, J=9.6, 2.9 Hz, 1H), 3.52-3.31 (m, 4H), 3.03 (dd, J=15.3, 5.9 Hz, 3H), 2.76-2.64 (m, 1H), 2.40-2.28 (m, 1H), 2.05-1.92 (m, 2H), 1.84-1.72 (in, 2H).

### Example 240: 5-Amino-3-[2-(1-cyclopropyl-6-fluoro-1,3-benzodiazol-5-yl)ethynyl]-1-[(3 S,5R)-5-[(1R)-1-hydroxyethyl]-1-(prop-2-enoyl)pyrrolidin-3-yl]pyrazole-4-carboxamide

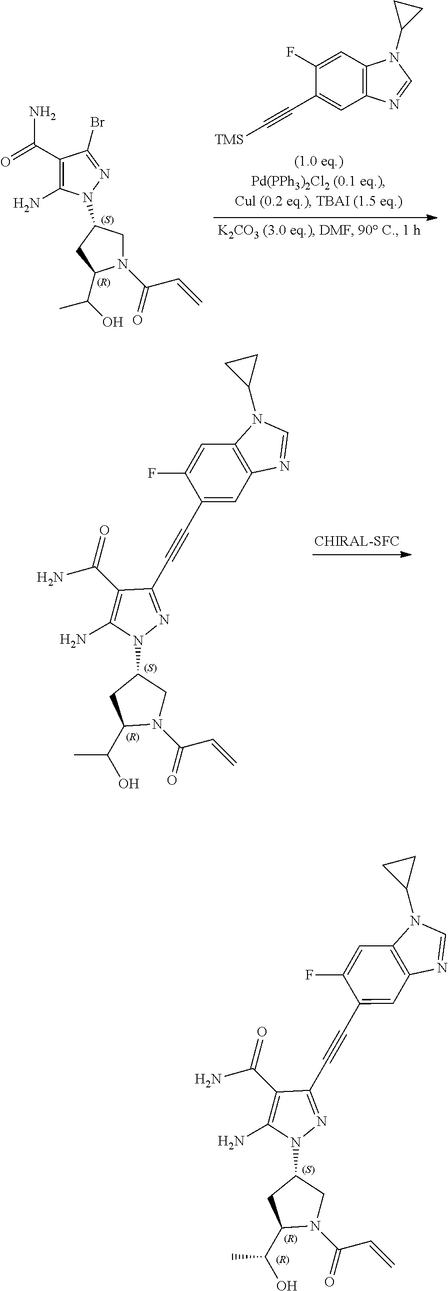

5-amino-3-[2-(1-cyclopropyl-6-fluoro-1,3-benzodiazol-5-yl)ethynyl]-1-[(3S,5R)-5-[(1R)-1-hydroxyethyl]-1-(prop-2-enoyl)pyrrolidin-3-yl]pyrazole-4-carboxamide. MS ESI calculated for C₂₅H₂₆FN₇O₃[M+H]⁺, 492.21, found 492.35; ¹H NMR (400 MHz, DMSO-d₆) δ 8.38 (s, 1H), 8.00-7.93 (m, 1H), 7.65 (d, J=9.20 Hz, 1H), 7.37 (s, 1H), 6.77-6.49 (m, 4H), 6.18-6.10 (m, 1H), 5.69-5.84 (m, 1H), 5.14-4.66 (m, 2H), 4.32-4.11 (m, 1H), 3.97-3.77 (m, 2H), 3.67-3.62 (m, 1H), 3.53-3.49 (m, 1H), 2.41-2.36 (m, 1H), 2.29-2.22 (m, 1H), 1.10-1.03 (m, 7H).

### Example 241: 5-Amino-3-[2-(1-cyclopropyl-6-fluoro-1,3-benzodiazol-5-yl)ethynyl]-1-[(3S,5R)-5-[(1S)-1-hydroxyethyl]-1-(prop-2-enoyl)pyrrolidin-3-yl]pyrazole-4-carboxamide

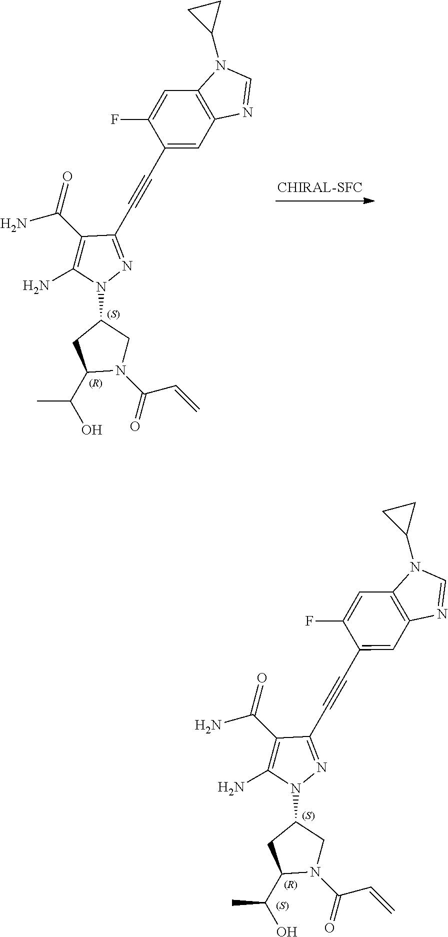

The racemic product 1-((3S,5R)-1-acryloyl-5-(1-hydroxyethyl)pyrrolidin-3-yl)-5-amino-3-((1-cyclopropyl-6-fluoro-1H-benzo[d]imidazol-5-yl)ethynyl)-1H-pyrazole-4-carboxamide (0.10 g) was purified by Prep-CHIRAL-HPLC with the following conditions Column: CHIRAL ART Cellulose-SC, 2×25 cm, 5 μm; Mobile Phase A: Hex (0.5% 2 M NH₃-MeOH)-HPLC, Mobile Phase B: MeOH:EtOH=1:1-HPLC; Flow rate: 20 mL/min; Gradient: 50% B to 50% B in 25 min; Wave Length: 220/254 nm; RT2: 22.72 min; Sample Solvent: MeOH:DCM=1:1; Injection Volume: 0.4 mL; The fractions contained desired product were combined and concentrated to afford 5-amino-3-[2-(1-cyclopropyl-6-fluoro-1,3-benzodiazol-5-yl)ethynyl]-1-[(3S,5R)-5-[(1S)-1-hydroxyethyl]-1-(prop-2-enoyl)pyrrolidin-3-yl]pyrazole-4-carboxamide (47.60 mg, 18%) as an off-white solid. MS ESI calculated for C₂₅H₂₆FN₇O₃[M+H]⁺, 492.21, found 492.30; ¹H NMR (400 MHz, DMSO-d₆) S 8.36 (s, 1H), 7.96 (d, J=6.40 Hz, 1H), 7.64 (d, J=10.40 Hz, 1H), 7.37 (s, 1H), 6.85-6.51 (m, 4H), 6.20-6.06 (m, 1H), 5.72-5.59 (m, 1H), 5.14-4.87 (m, 2H), 4.46-4.18 (m, 1H), 4.11-3.77 (m, 2H), 3.72-3.58 (m, 1H), 3.54-3.48 (m, 1H), 2.48-2.38 (m, 1H), 2.30 (s, 1H), 1.15-0.99 (m, 7H).

### Example 242: 5-Amino-3-[2-(6-chloro-1-cyclopropyl-1,3-benzodiazol-5-yl)ethynyl]-1-[(3 S,5R)-5-[(1R)-1-hydroxyethyl]-1-(prop-2-enoyl)pyrrolidin-3-yl]pyrazole-4-carboxamide

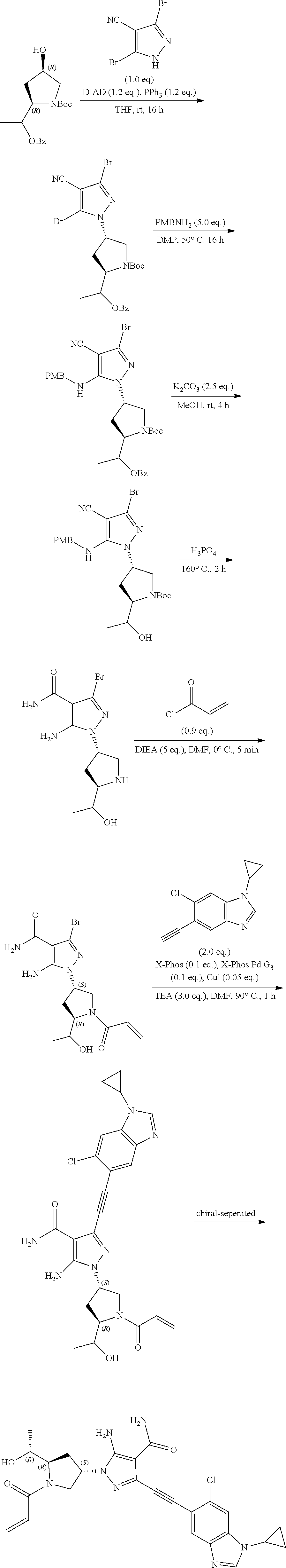

5-amino-3-[2-(6-chloro-1-cyclopropyl-1,3-benzodiazol-5-yl)ethynyl]-1-[(3S,5R)-5-[(1R)-1-hydroxyethyl]-1-(prop-2-enoyl)pyrrolidin-3-yl]pyrazole-4-carboxamide. MS ESI calculated for C₂₅H₂₆ClN₇O₃[M+H]+, 508.18, found 508.40; ¹H NMR (400 MHz, CDCl₃) δ 8.40 (s, 1H), 8.00 (s, 1H), 7.90 (s, 1H), 6.71-6.51 (m, 1H), 6.19-6.11 (m, 1H), 5.72-5.57 (m, 1H), 5.03 (s, 1H), 4.27-4.14 (m, 1H), 3.90-3.64 (m, 3H), 3.54-3.50 (m, 1H), 2.41-2.26 (m, 2H), 1.13-1.03 (in, 7H₁).

### Example 243: 5-Amino-3-[2-(6-chloro-1-cyclopropyl-1,3-benzodiazol-5-yl)ethynyl]-1-[(3 S,5R)-5-[(1 S)-1-hydroxyethyl]-1-(prop-2-enoyl)pyrrolidin-3-yl]pyrazole-4-carboxamide

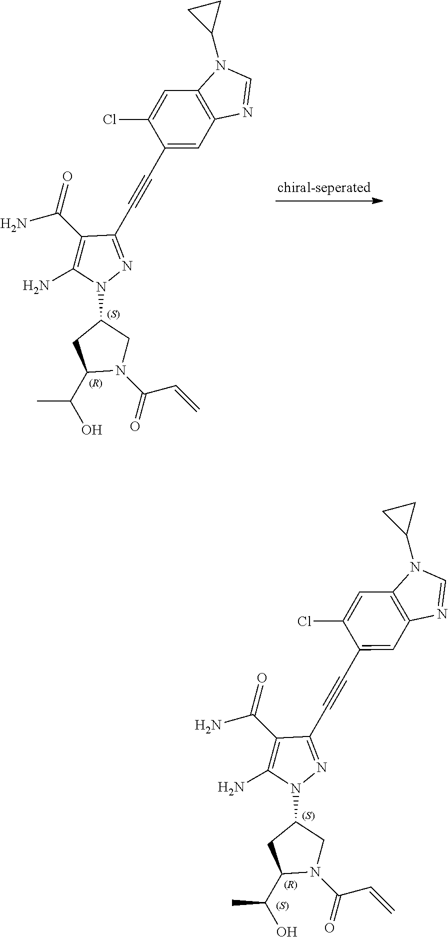

5-amino-3-[2-(6-chloro-1-cyclopropyl-1,3-benzodiazol-5-yl)ethynyl]-1-[(3S,5R)-5-[(15)-1-hydroxyethyl]-1-(prop-2-enoyl)pyrrolidin-3-yl]pyrazole-4-carboxamide. MS ESI calculated for C₂₅H₂₆ClN₇O₃[M+H]+, 508.18, found 508.40; ¹H NMR (400 MHz, CDCl₃) δ 8.40 (s, 1H), 8.01 (s, 1H), 7.90 (s, 1H), 6.80-6.51 (i, 1H), 6.16-6.10 (m, 1H), 5.71-5.63 (m, 1H), 5.05 (s, 1H), 4.41 (s, 1H), 4.05-3.84 (m, 2H), 3.55-3.50 (m, 1H), 2.67-2.47 (m, 1H), 2.45-2.25 (m, 2H), 1.14-1.08 (m, 4H), 1.06-1.01 (m, 3H).

### Example 244: 1-((3S,5R)-1-Acryloyl-5-((R)-1-hydroxyethyl)pyrrolidin-3-yl)-5-amino-3-((6-chloro-1-cyclopropyl-2-methyl-1H-benzo[d]imidazol-5-yl)ethynyl)-1H-pyrazole-4-carboxamide

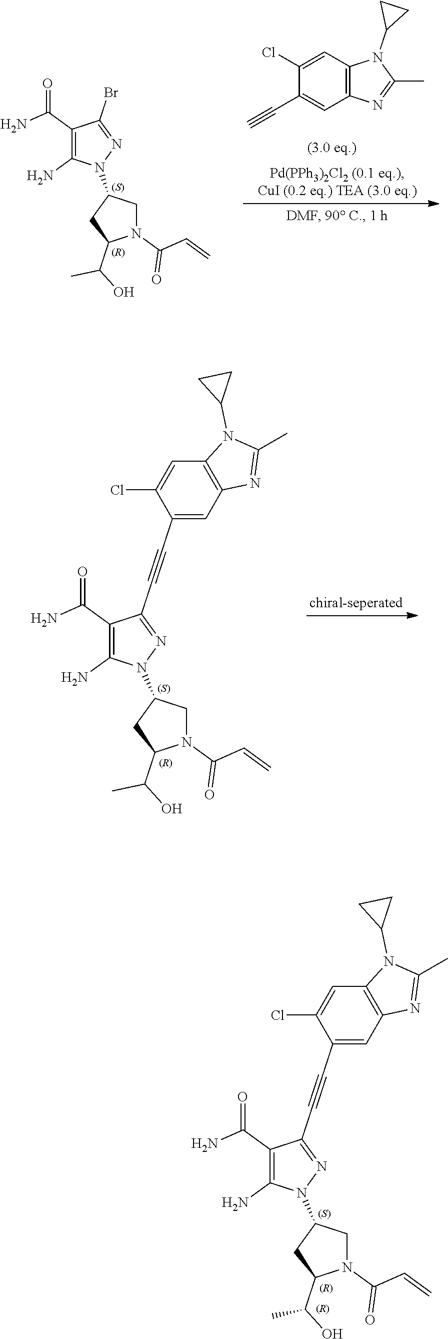

1-((3S,5R)-1-acryloyl-5-((R)-1-hydroxyethyl)pyrrolidin-3-yl)-5-amino-3-((6-chloro-1-cyclopropyl-2-methyl-1H-benzo[d]imidazol-5-yl)ethynyl)-1H-pyrazole-4-carboxamide. MS ESI calculated for C₂₆H₂₈ClN₇O₃[M+H]+, 522.19, found 522.25; ¹H NMR (400 MHz, Chloroform-d) δ 7.91 (s, 1H), 7.55 (s, 1H), 7.14 (s, 1H), 6.56-6.27 (m, 2H), 5.94-5.49 (m, 3H), 5.11 (s, 1H), 4.52-3.86 (m, 4H), 3.36-3.06 (m, 1H), 2.69-2.65 (m, 3H), 2.56 (d, J=11.6 Hz, 1H), 2.38 (t, J=9.7 Hz, HH), 1.31-1.18 (i, 6H), 1.07 (s, 2H).

### Example 245: 1-((3S,5R)-1-Acryloyl-5-((S)-1-hydroxyethyl)pyrrolidin-3-yl)-5-amino-3-((6-chloro-1-cyclopropyl-2-methyl-1H-benzo[d]imidazol-5-yl)ethynyl)-1H-pyrazole-4-carboxamide

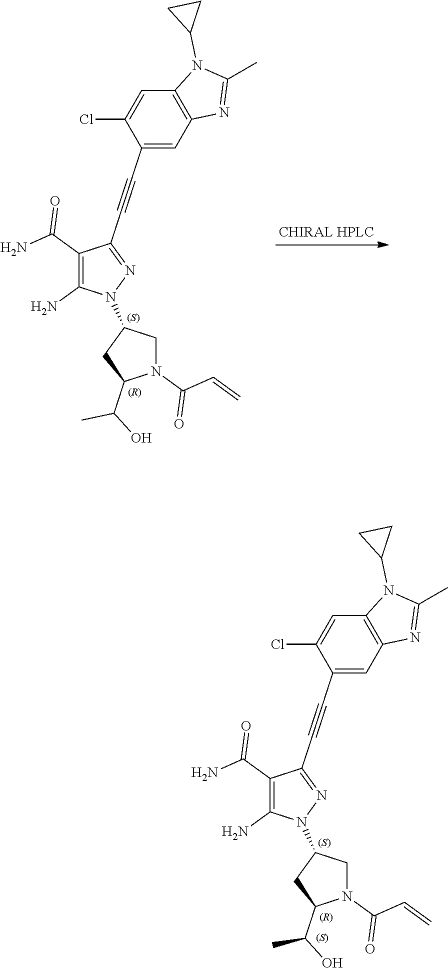

1-((3S,5R)-1-acryloyl-5-((S)-1-hydroxyethyl)pyrrolidin-3-yl)-5-amino-3-((6-chloro-1-cyclopropyl-2-methyl-1H-benzo[d]imidazol-5-yl)ethynyl)-1H-pyrazole-4-carboxamide. MS ESI calculated for C₂₆H₂₈ClN₇O₃[M+H]⁺, 522.19, found 522.20; ¹H NMR (400 MHz, DMSO-d₆) δ 7.86 (s, 1H), 7.72 (s, 1H), 7.38 (s, 1H), 6.87-6.48 (m, 4H), 6.17-6.10 (m, 1H), 5.69-5.62 (m, 1H), 5.18-4.86 (m, 2H), 4.52-4.17 (m, 1H), 4.06-3.98 (m, 1H), 3.91-3.85 (m, 1H), 3.69-3.62 (m, 1H), 3.35 (dd, J=7.1, 3.8 Hz, 1H), 2.60 (s, 3H), 2.48-2.27 (m, 2H), 1.26-1.15 (m, 2H), 1.14-0.96 (m, 5H).

### Example 246: 5-Amino-3-[2-(1-cyclopropyl-6-fluoro-2-methyl-1,3-benzodiazol-5-yl)ethynyl]-1-[(3 S,5R)-5-[(1R)-1-hydroxyethyl]-1-(prop-2-enoyl)pyrrolidin-3-yl]pyrazole-4-carboxamide

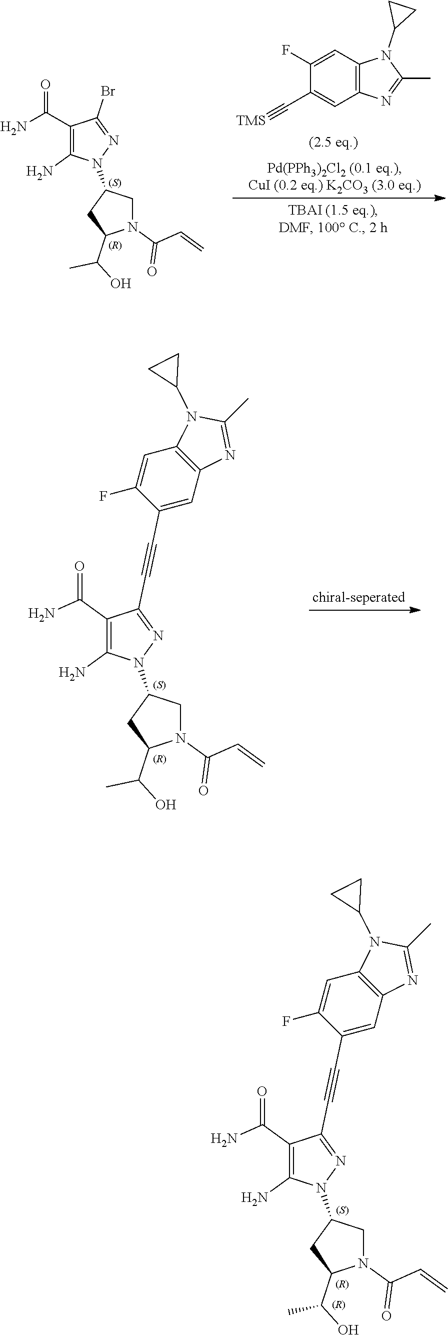

5-amino-3-[2-(1-cyclopropyl-6-fluoro-2-methyl-1,3-benzodiazol-5-yl)ethynyl]-1-[(3S,5R)-5-[(1R)-1-hydroxyethyl]-1-(prop-2-enoyl)pyrrolidin-3-yl]pyrazole-4-carboxamide. MS ESI calculated for C₂₆H₂₈FN₇O₃[M+H]⁺, 506.22, found 506.35; ¹H NMR (400 MHz, CDCl₃) δ 7.83 (d, J=4 Hz, 1H), 7.25 (s, 1H), 7.09 (s, 1H), 6.50-6.38 (m, 2H), 5.75-5.72 (m, 3H), 5.68 (s, 1H), 5.53 (s, 1H), 4.47-4.38 (m, 2H), 4.15-4.04 (m, 2H), 2.39 (s, 1H), 2.71 (s, 3H), 2.63-2.55 (m, 1H), 2.39 (s, 1H), 2.30-2.26 (m, 2H), 2.24-2.21 (m, 3H), 1.08 (s, 2H).

### Example 247: 5-Amino-3-[2-(1-cyclopropyl-6-fluoro-2-methyl-1,3-benzodiazol-5-yl)ethynyl]-1-[(3S,5R)-5-[(1S)-1-hydroxyethyl]-1-(prop-2-enoyl)pyrrolidin-3-yl]pyrazole-4-carboxamide

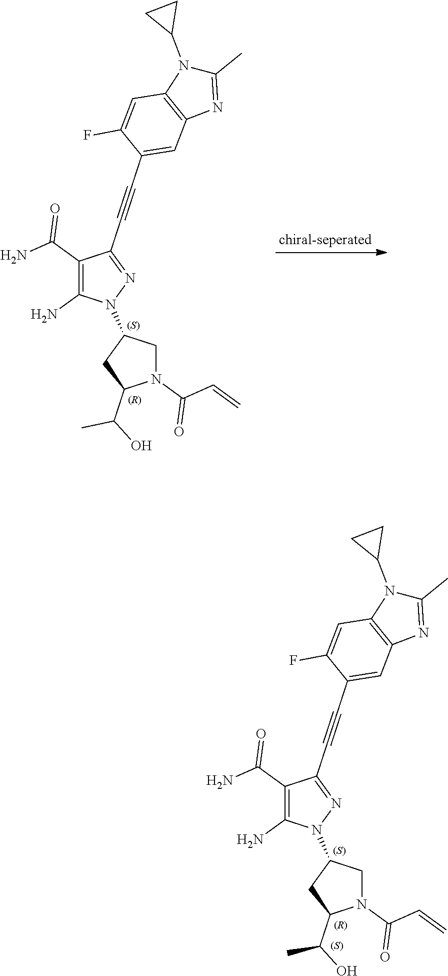

5-amino-3-[2-(1-cyclopropyl-6-fluoro-2-methyl-1,3-benzodiazol-5-yl)ethynyl]-1-[(3S,5R)-5-[(1S)-1-hydroxyethyl]-1-(prop-2-enoyl)pyrrolidin-3-yl]pyrazole-4-carboxamide. MS ESI calculated for C₂₆H₂₈FN₇O₃[M+H]⁺, 506.22, found 506.35; ¹H NMR (400 MHz, CDCl₃) δ 7.82 (d, J=4 Hz, 1H), 7.26-7.23 (m, 1H), 7.11 (s, 1H), 6.45-6.40 (m, 2H), 5.79-5.73 (m, 1H), 5.73 (s, 2H), 5.68 (s, 1H), 5.50 (s, 1H), 4.86-4.68 (m, 1H), 4.52-4.45 (m, 1H), 4.15-4.12 (m, 1H), 4.05-4.01 (m, 1H), 3.91-3.84 (m, 1H), 3.28-3.21 (m, 1H), 2.76-2.69 (m, 4H), 2.31-2.15 (m, 1H), 1.32-1.21 (m, 4H), 1.09-1.05 (m, 2H).

### Example 248: 3-{2-[6-Chloro-1-(oxetan-3-yl)-1,3-benzodiazol-5-yl]ethynyl}-1-[(3S,5R)-5-(methoxymethyl)-1-(prop-2-enoyl)pyrrolidin-3-yl]-5-(methylamino)pyrazole-4-carboxamide

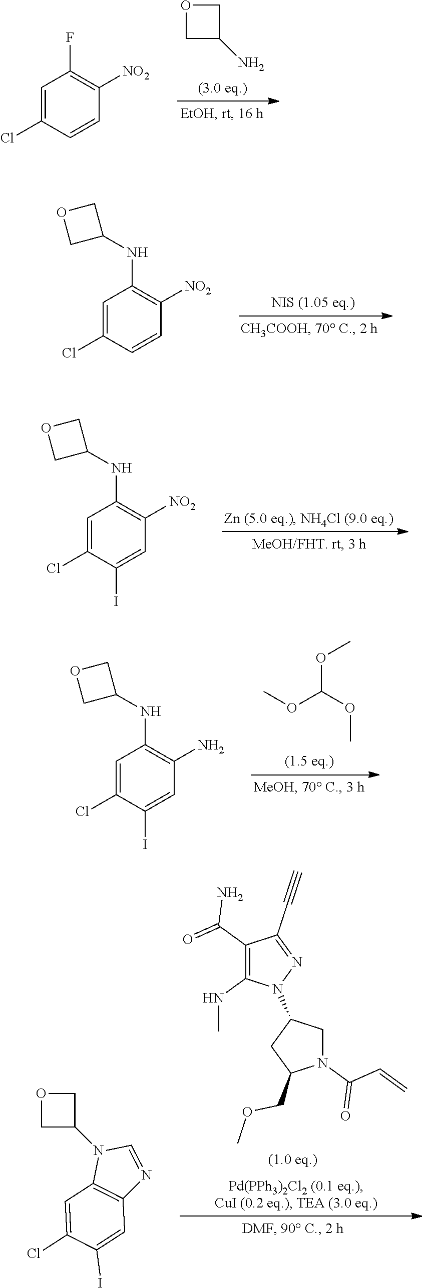

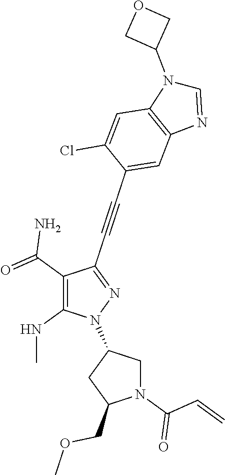 [SMILES: C=CC(=O)N1C[C@@H](n2nc(C#Cc3cc4ncn(C5COC5)c4cc3Cl)c(C(N)=O)c2NC)C[C@@H]1COC]

3-{2-[6-chloro-1-(oxetan-3-yl)-1,3-benzodiazol-5-yl]ethynyl}-1-[(3S,5R)-5-(methoxymethyl)-1-(prop-2-enoyl)pyrrolidin-3-yl]-5-(methylamino)pyrazole-4-carboxamide. MS ESI calculated for C₂₆H₂₈ClN₇O₄[M+H]⁺, 538.19, found 538.25; ¹H NMR (400 MHz, Chloroform-d) δ 8.12 (d, J=5.1 Hz, 2H), 7.88 (d, J=4.6 Hz, 1H), 7.20-7.15 (m, 1H), 6.83 (s, 1H), 6.55-6.35 (m, 2H), 5.76-5.67 (m, 1H), 5.51-5.48 (m, 2H), 5.45 (s, 1H), 5.27 (t, J=7.6 Hz, 2H), 5.08-5.01 (m, 2H), 4.56 (d, J=9.0 Hz, 1H), 4.11 (t, J=9.1 Hz, 1H), 4.06-3.94 (m, 1H), 3.90-3.87 (m, 1H), 3.57-3.41 (m, 1H), 3.37 (d, J=5.2 Hz, 3H), 3.03 (d, J=15.6 Hz, 3H), 2.78-2.65 (m, 1H), 2.34-2.31 (m, 1H).

### Example 249: 3-{2-[6-Chloro-2-methyl-1-(oxetan-3-yl)-1,3-benzodiazol-5-yl]ethynyl}-1-[(3S,5R)-5-(methoxymethyl)-1-(prop-2-enoyl)pyrrolidin-3-yl]-5-(methylamino)pyrazole-4-carboxamide

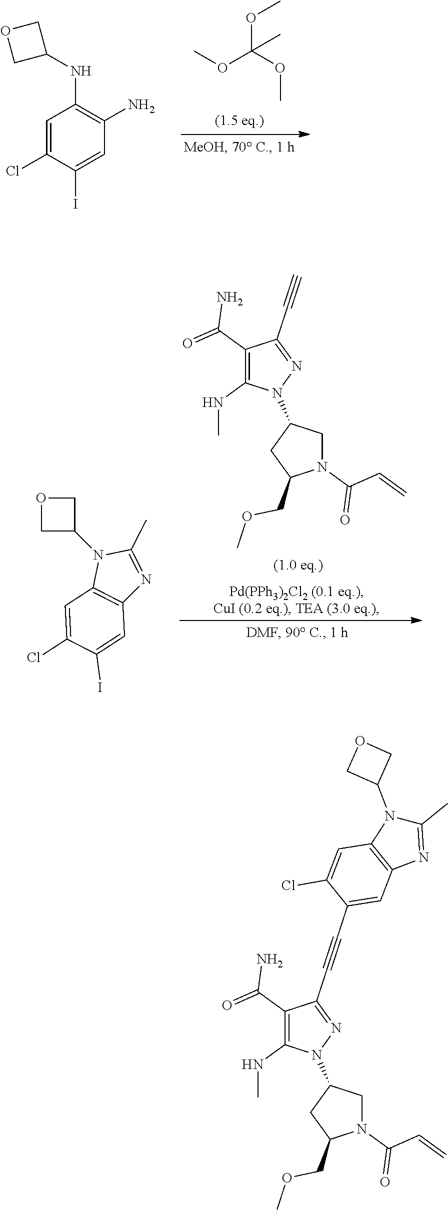

3-{2-[6-chloro-2-methyl-1-(oxetan-3-yl)-1,3-benzodiazol-5-yl]ethynyl}-1-[(3S,5R)-5-(methoxymethyl)-1-(prop-2-enoyl)pyrrolidin-3-yl]-5-(methylamino)pyrazole-4-carboxamide. MS ESI calculated for C₂₇H₃₀ClN₇O₄[M+H]⁺, 552.20, found 552.25; ¹H NMR (400 MHz, Chloroform-d) δ 8.13 (d, J=3.0 Hz, 1H), 7.99 (d, J=5.8 Hz, 1H), 7.22 (s, 1H), 6.83 (d, J=6.0 Hz, 1H), 6.55-6.35 (m, 2H), 5.71-5.65 (m, 1H), 5.58-5.44 (m, 2H), 5.26 (t, J=7.7 Hz, 2H), 5.17-5.10 (m, 2H), 4.56 (d, J=9.1 Hz, 1H), 4.10-4.05 (m, 1H), 4.03 (t, J=9.3 Hz, 1H), 3.90-3.84 (m, 1H), 3.55-3.40 (m, 1H), 3.37 (d, J=5.0 Hz, 3H), 3.03-2.94 (m, 3H), 2.78-2.65 (m, 1H), 2.60 (s, 3H), 2.34-2.28 (m, 1H).

### Example 250: 3-[2-(1-Cyclopropyl-6-fluoro-1,3-benzodiazol-5-yl)ethynyl]-5-[(2-hydroxyethyl)amino]-1-[(3S,5R)-5-(methoxymethyl)-1-(prop-2-enoyl)pyrrolidin-3-yl]pyrazole-4-carboxamide

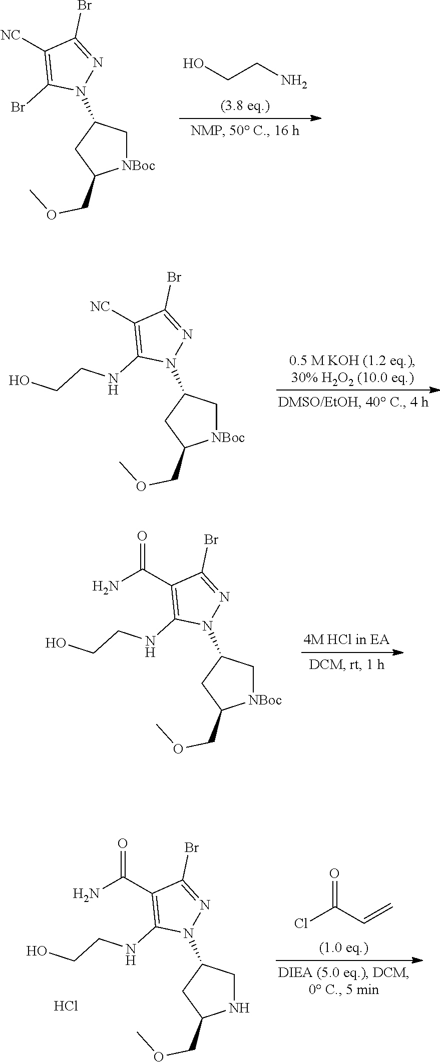

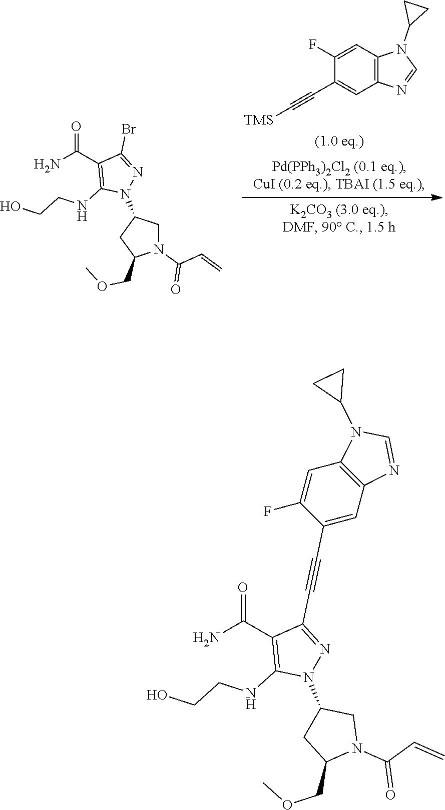 [SMILES: C[Si](C)(C)C#Cc1cc2ncn(C3CC3)c2cc1F ; C=CC(=O)N1C[C@@H](n2nc(Br)c(C(N)=O)c2NCCO)C[C@@H]1COC ; C=CC(=O)N1C[C@@H](n2nc(C#Cc3cc4ncn(C5CC5)c4cc3F)c(C(N)=O)c2NCCO)C[C@@H]1COC]

3-[2-(1-cyclopropyl-6-fluoro-1,3-benzodiazol-5-yl)ethynyl]-5-[(2-hydroxyethyl)amino]-1-[(3S,5R)-5-(methoxymethyl)-1-(prop-2-enoyl)pyrrolidin-3-yl]pyrazole-4-carboxamide. MS ESI calculated for C₂₇H₃₀FN₇O₄[M+H]+, 536.24, found 536.50; ¹H NMR (400 MHz, CDCl₃) δ 8.02-7.94 (m, 2H), 7.32 (d, J=9.20 Hz, 2H), 6.53-6.37 (m, 2H), 5.88-5.21 (m, 4H), 4.58 (d, J=9.20 Hz, 1H), 4.07-3.81 ((, 3H), 3.71-3.41 (m, 4H), 3.39-3.22 (m, 6H), 2.73-2.61 (m, 1H), 2.40-2.24 (m, 1H), 1.21-1.07 (m, 2H), 1.06 (d, J=7.20 Hz, 2H).

### Example 251: 3-[2-(1-Cyclopropyl-4,6-difluoro-1,3-benzodiazol-5-yl)ethynyl]-5-[(2-hydroxyethyl)amino]-1-[(3 S,5R)-5-(methoxymethyl)-1-(prop-2-enoyl)pyrrolidin-3-yl]pyrazole-4-carboxamide

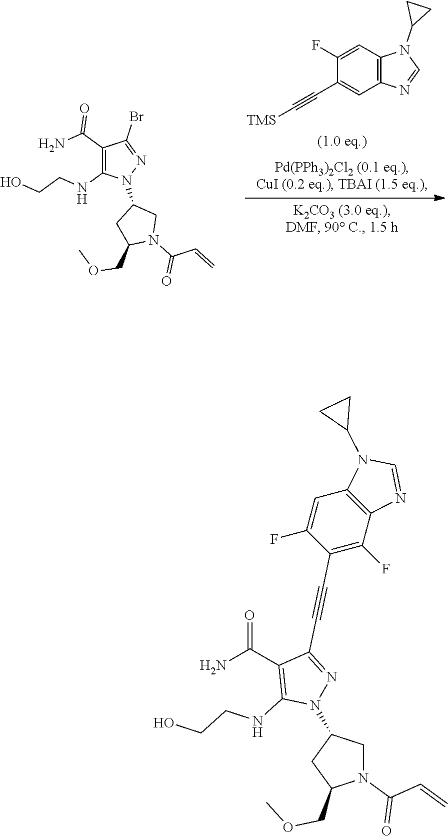 [SMILES: C=CC(=O)N1C[C@@H](n2nc(Br)c(C(N)=O)c2NCCO)C[C@@H]1COC ; C[Si](C)(C)C#Cc1cc2ncn(C3CC3)c2cc1F ; C=CC(=O)N1C[C@@H](n2nc(C#Cc3c(F)cc4c(ncn4C4CC4)c3F)c(C(N)=O)c2NCCO)C[C@@H]1COC]

3-[2-(1-cyclopropyl-4,6-difluoro-1,3-benzodiazol-5-yl)ethynyl]-5-[(2-hydroxyethyl)amino]-1-[(3S,5R)-5-(methoxymethyl)-1-(prop-2-enoyl)pyrrolidin-3-yl]pyrazole-4-carboxamide. MS ESI calculated for C₂₇H₂₉F₂N₇O₄[M+H]⁺, 554.22, found 554.25; ¹H NMR (400 MHz, CDCl₃) δ 7.99 (s, 1H), 7.17 (d, J=8.00 Hz, 1H), 6.56-6.38 (m, 2H), 5.76-5.67 (m, 2H), 5.48-5.39 (m, 1H), 4.62-4.39 (m, 1H), 4.03 (d, J=8.10 Hz, 2H), 3.88-3.85 (m, 1H), 3.67 (s, 2H), 3.52-3.42 (m, 1H), 3.38 (d, J=3.20 Hz, 4H), 3.32 (s, 2H), 2.96-2.60 (m, 1H), 2.39-2.25 (m, 1H), 1.25-1.20 (m, 2H), 1.08 (s, 2H).

### Example 252: 3-[2-(1-Cyclopropyl-6-fluoro-2-methyl-1,3-benzodiazol-5-yl)ethynyl]-5-[(2-hydroxyethyl)amino]-1-[(3S,5R)-5-(methoxymethyl)-1-(prop-2-enoyl)pyrrolidin-3-yl]pyrazole-4-carboxamide

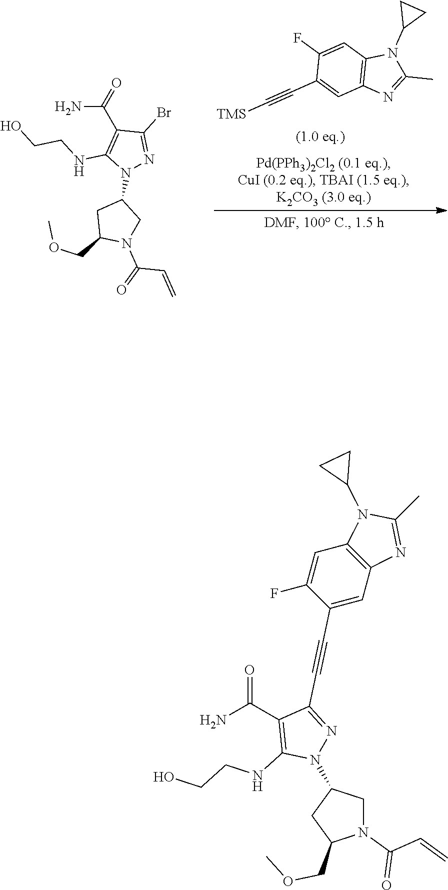 [SMILES: C=CC(=O)N1C[C@@H](n2nc(Br)c(C(N)=O)c2NCCO)C[C@@H]1COC ; Cc1nc2cc(C#C[Si](C)(C)C)c(F)cc2n1C1CC1 ; C=CC(=O)N1C[C@@H](n2nc(C#Cc3cc4nc(C)n(C5CC5)c4cc3F)c(C(N)=O)c2NCCO)C[C@@H]1COC]

3-[2-(1-cyclopropyl-6-fluoro-2-methyl-1,3-benzodiazol-5-yl)ethynyl]-5-[(2-hydroxyethyl)amino]-1-[(3S,5R)-5-(methoxymethyl)-1-(prop-2-enoyl)pyrrolidin-3-yl]pyrazole-4-carboxamide. MS ESI calculated for C₂₈H₃₂FN₇O₄ [M+H]⁺, 550.25, found 550.40; ¹H NMR (400 MHz, CDCl₃) δ 7.79 (d, J=4 Hz, 1H), 7.50 (s, 2H), 6.79-6.58 (m, 3H), 6.23-6.15 (m, 1H), 5.71-5.68 (m, 1H), 5.67-5.25 (m, 1H), 4.90-4.86 (m, 1H), 4.50-4.45 (m, 1H), 4.05-4.01 (m, 1H), 3.90-3.73 (m, 1H), 3.62-3.56 (m, 3H), 3.49-3.44 (m, 2H), 3.36-3.27 (m, 5H), 2.75-2.59 (m, 3H), 2.44 (d, J=4 Hz, 1H), 2.30-2.26 (m, 1H), 1.22-1.17 (m, 2H), 1.06-1.02 (m, 2H).

### Example 253: 1-((3S,5R)-1-Acryloyl-5-(methoxymethyl)pyrrolidin-3-yl)-3-((6-chloro-1-cyclopropyl-2-methyl-1H-benzo[d]imidazol-5-yl)ethynyl)-5-((2-hydroxyethyl)amino)-1H-pyrazole-4-carboxamide

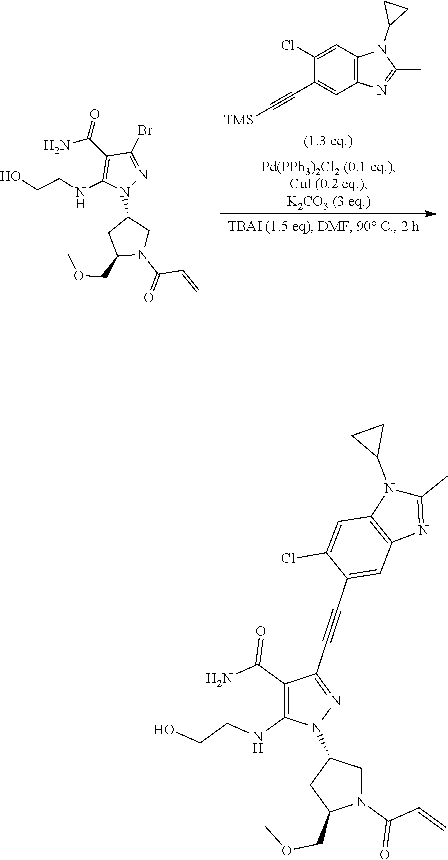 [SMILES: C=CC(=O)N1C[C@@H](n2nc(Br)c(C(N)=O)c2NCCO)C[C@@H]1COC ; Cc1nc2cc(C#C[Si](C)(C)C)c(Cl)cc2n1C1CC1 ; C=CC(=O)N1C[C@@H](n2nc(C#Cc3cc4nc(C)n(C5CC5)c4cc3Cl)c(C(N)=O)c2NCCO)C[C@@H]1COC]

1-((3S,5R)-1-acryloyl-5-(methoxymethyl)pyrrolidin-3-yl)-3-((6-chloro-1-cyclopropyl-2-methyl-1H-benzo[d]imidazol-5-yl)ethynyl)-5-((2-hydroxyethyl)amino)-1H-pyrazole-4-carboxamide. MS ESI calculated for C₂₈H₃₂ClN₇O₄ [M+H]⁺, 566.22, found 566.35; ¹H NMR (400 MHz, CDCl₃) δ 7.93 (d, J=5.6 Hz, 1H), 7.58 (s, 1H), 7.44 (s, 1H), 6.44-6.42 (m, 2H), 5.86-5.70 (m, 2H), 5.60 (s, 1H), 5.48-5.44 (m, 1H), 4.61-4.58 (m, 1H), 4.06-3.95 (m, 2H), 3.87 (dd, J=9.8, 3.2 Hz, 1H), 3.66 (s, 2H), 3.47-3.42 (m, 1H), 3.38 (d, J=2.5 Hz, 3H), 3.31 (s, 2H), 3.21-3.16 (m, 1H), 2.95-2.68 (m, 4H), 2.32-2.30 (m, 1H), 1.29-1.25 (m, 2H), 1.10-1.02 (in, 2H).

### Example 254: 3-[2-(6-Fluoro-2H-1,3-benzodioxol-5-yl)ethynyl]-1-[(3S,5R)-5-(methoxymethyl)-1-(prop-2-enoyl)pyrrolidin-3-yl]-5-(methylamino)pyrazole-4-carboxamide

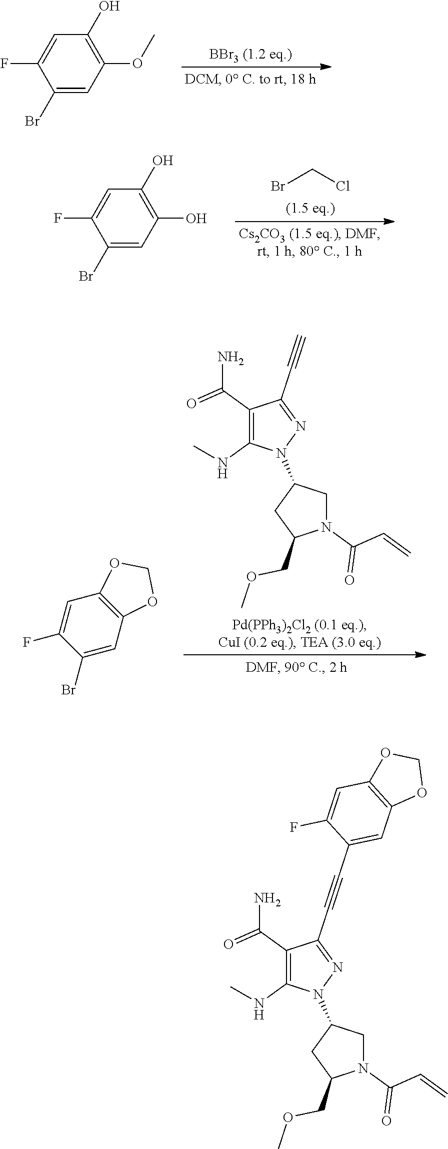

3-[2-(6-fluoro-2H-1,3-benzodioxol-5-yl)ethynyl]-1-[(3S,5R)-5-(methoxymethyl)-1-(prop-2-enoyl)pyrrolidin-3-yl]-5-(methylamino)pyrazole-4-carboxamide. MS ESI calculated for C₂₃H₂₄FN₅O₅[M+H]⁺, 470.18, found 470.20; ¹H NMR (400 MHz, Chloroform-d) δ 6.91 (d, J=5.8 Hz, 1H), 6.80 (d, J=6.2 Hz, 1H), 6.64 (d, J=8.7 Hz, 1H), 6.52-6.34 (m, 2H), 6.03 (s, 2H), 5.70-5.68 (m, 1H), 5.48-5.46 (m, 1H), 5.29 (s, 1H), 4.55 (d, J=9.0 Hz, 1H), 4.07 (t, J=8.9 Hz, 1H), 4.00 (t, J=9.1 Hz, 1H), 3.89-3.85 (m, 1H), 3.52-3.40 (m, 1H), 3.36 (d, J=4.6 Hz, 3H), 3.01-2.98 (m, 3H), 2.69-2.62 (m, 1H), 2.29-2.21 (m, 1H), 1.26 (s, 1H).

### Example 256: 3-[2-(3-Chloro-6-fluoroquinolin-7-yl)ethynyl]-1-[(3S,5R)-5-(methoxymethyl)-1-(prop-2-enoyl)pyrrolidin-3-yl]-5-(methylamino)pyrazole-4-carboxamide

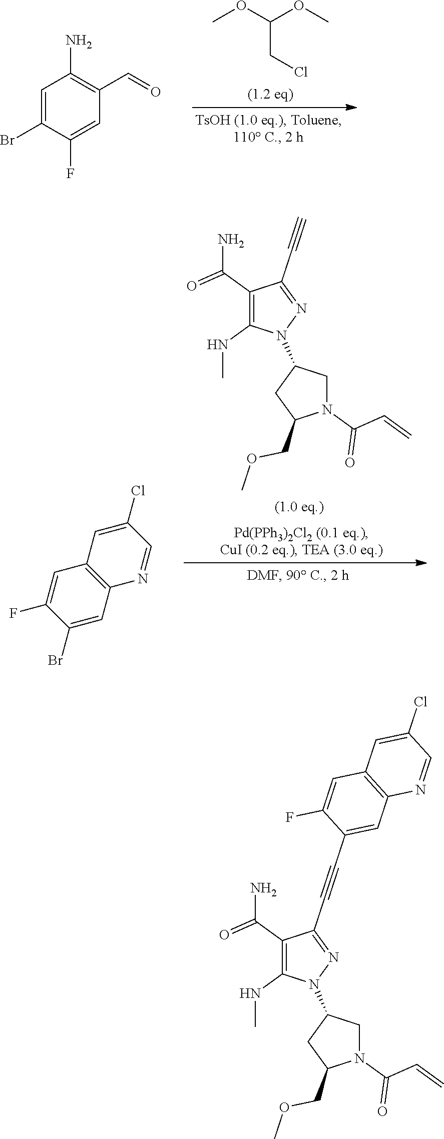

3-[2-(3-chloro-6-fluoroquinolin-7-yl)ethynyl]-1-[(3S,5R)-5-(methoxymethyl)-1-(prop-2-enoyl)pyrrolidin-3-yl]-5-(methylamino)pyrazole-4-carboxamide. MS ESI calculated for C₂₅H₂₄ClFN₆O₃[M+H]⁺, 511.16, found 511.15; ¹H NMR (400 MHz, Chloroform-d) δ 8.82 (d, J=2.3 Hz, 1H), 8.32 (t, J=5.5 Hz, 1H), 8.08 (d, J=2.4 Hz, 1H), 7.44 (d, J=9.4 Hz, 1H), 6.96 (s, 1H), 6.84 (s, 1H), 6.58-6.36 (m, 2H), 5.72-5.70 (m, 1H), 5.58-5.45 (m, 1H), 5.39 (s, 1H), 4.57 (d, J=9.0 Hz, 1H), 4.10 (t, J=8.9 Hz, 1H), 4.09-3.97 (m, 1H), 4.00-3.87 (m, 1H), 3.54-3.41 (m, 1H), 3.37 (d, J=5.0 Hz, 3H), 3.04 (d, J=15.2 Hz, 3H), 2.70-2.68 (m, 1H), 2.35-2.31 (m, 1H).

### Example 257: 1-((3S,5R)-1-Acryloyl-5-(methoxymethyl)pyrrolidin-3-yl)-3-((6-fluoro-3-methylimidazo[1,2-a]pyridin-7-yl)ethynyl)-5-(methylamino)-1H-pyrazole-4-carboxamide

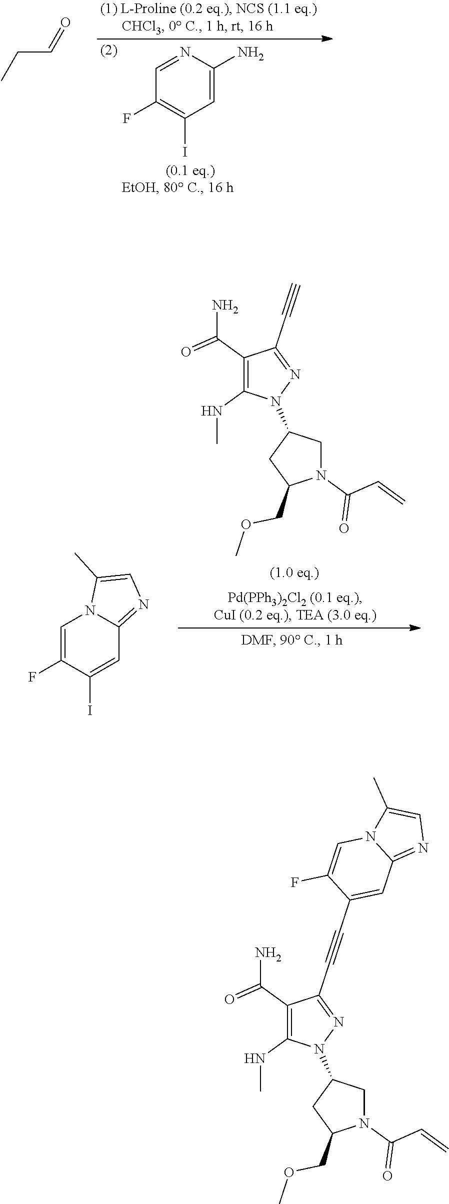

1-((3S,5R)-1-Acryloyl-5-(methoxymethyl)pyrrolidin-3-yl)-3-((6-fluoro-3-methylimidazo[1,2-a]pyridin-7-yl)ethynyl)-5-(methylamino)-1H-pyrazole-4-carboxamide. MS ESI calculated for C₂₄H₂₆FN₇O₃[M+H]⁺, 480.21, found 480.55; ¹H NMR (400 MHz, Chloroform-d) δ 6.92-6.85 (m, 2H), 6.42 (d, J=8.2 Hz, 2H), 5.71 (s, 1H), 5.51 (s, 1H), 5.30 (s, 1H), 4.62-4.35 (m, 1H), 4.16-3.96 (m, 2H), 3.90 (d, J=9.5 Hz, 1H), 3.51 (s, 1H), 3.45 (s, 1H), 3.43 (s, 1H), 3.37 (d, J=4.6 Hz, 4H), 3.09-3.00 (m, 3H), 2.70 (d, J=10.8 Hz, 1H), 2.32 (s, 1H).

### Example 258: 3-[2-(6-Chloro-3-methylquinolin-7-yl)ethynyl]-1-[(3S,5R)-5-(methoxymethyl)-1-(prop-2-enoyl)pyrrolidin-3-yl]-5-(methylamino)pyrazole-4-carboxamide

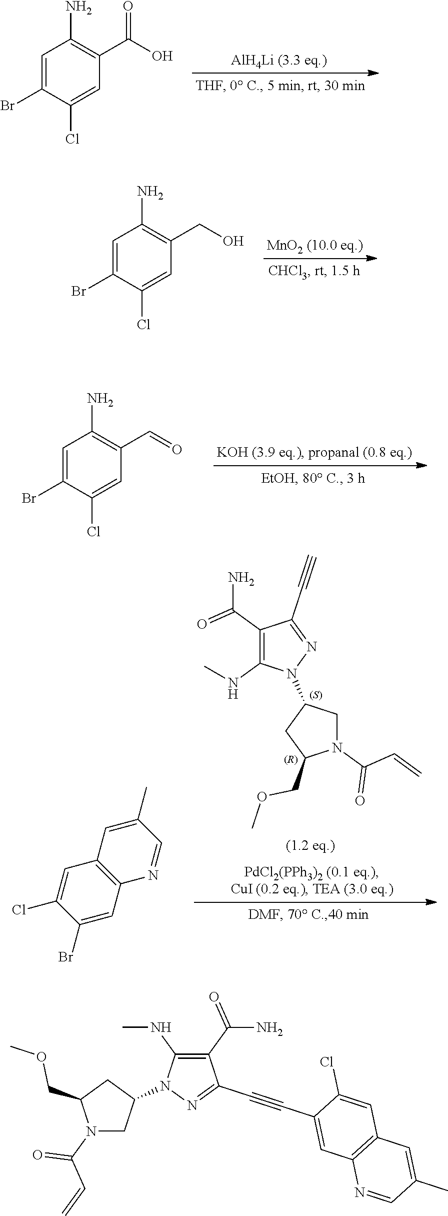

3-[2-(6-Chloro-3-methylquinolin-7-yl)ethynyl]-1-[(3S,5R)-5-(methoxymethyl)-1-(prop-2-enoyl)pyrrolidin-3-yl]-5-(methylamino)pyrazole-4-carboxamide. MS ESI calculated for C₂₆H₂₇ClN₆O₃[M+H]⁺, 507.18, found 507.50; ¹H NMR (400 MHz, DMSO-d₆) δ 8.86 (d, J=2.1 Hz, 1H), 8.31 (s, 1H), 8.20 (s, 1H), 8.14 (s, 1H), 7.54 (s, 1H), 6.90 (s, 1H), 6.78-6.55 (m, 2H), 6.17-6.13 (m, 1H), 5.70-5.60 (m, 1H), 5.28-5.18 (m, 1H), 4.64-4.31 (m, 1H), 4.09-3.67 (m, 2H), 3.63-3.42 (m, 2H), 3.31 (s, 3H), 2.96-2.86 (m, 3H), 2.62-2.52 (m, 1H), 2.50-2.40 (m, 3H), 2.34-2.25 (m, 1H).

### Example 259: 3-[2-(1-Cyclopropyl-6-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide

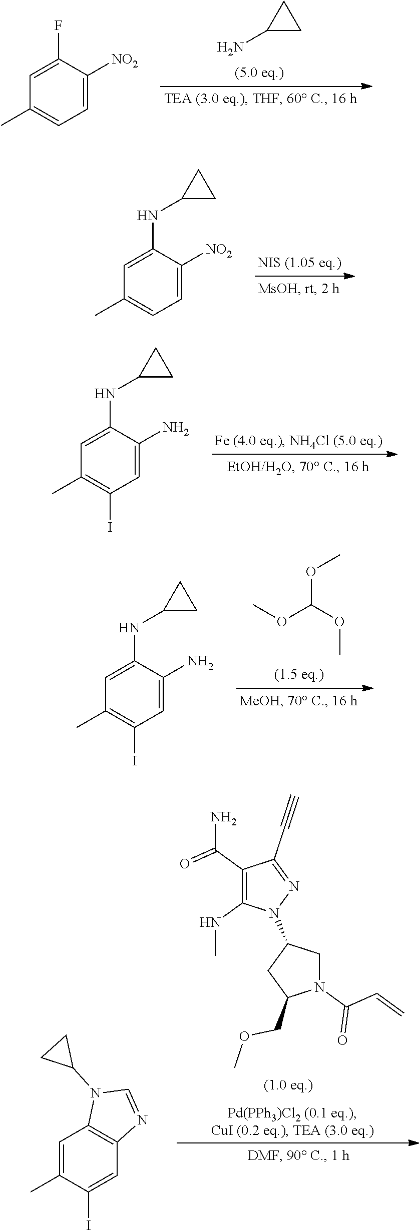

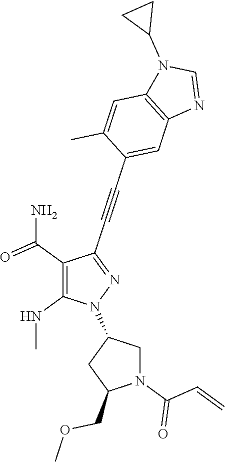 [SMILES: C=CC(=O)N1C[C@@H](n2nc(C#Cc3cc4ncn(C5CC5)c4cc3C)c(C(N)=O)c2NC)C[C@@H]1COC]

3-[2-(1-cyclopropyl-6-methyl-1,3-benzodiazol-5-yl)ethynyl]-1-[(3S,5R)-5-(methoxymethyl)-1-(prop-2-enoyl)pyrrolidin-3-yl]-5-(methylamino)pyrazole-4-carboxamide. MS ESI calculated for C₂₇H₃₁N₇O₃ [M+H]⁺, 502.25, found 502.30; ¹H NMR (400 MHz, CDCl₃) δ 8.25 (s, 1H), 7.83 (s, 1H), 7.58 (s, 1H), 7.38 (s, 1H), 6.81-6.59 (m, 3H), 6.20-6.15 (m, 1H), 5.71-5.67 (m, 1H), 5.29-5.21 (m, 1H), 4.54-4.40 (m, 1H), 4.05-3.72 (m, 2H), 3.63-3.45 (m, 3H), 3.20 (s, 3H), 2.95 (t, J=12 Hz, 3H), 2.65-2.45 (m, 3H), 2.32-2.26 (m, 1H), 1.13-1.07 (m, 2H), 1.05-1.01 (m, 2H).

### Example 260: 1-((3S,5R)-1-acryloyl-5-(methoxymethyl)pyrrolidin-3-yl)-3-((7-fluoro-2,3-dihydro-1H-benzo[d]pyrrolo[1,2-a]imidazol-6-yl)ethynyl)-5-(methylamino)-1H-pyrazole-4-carboxamide

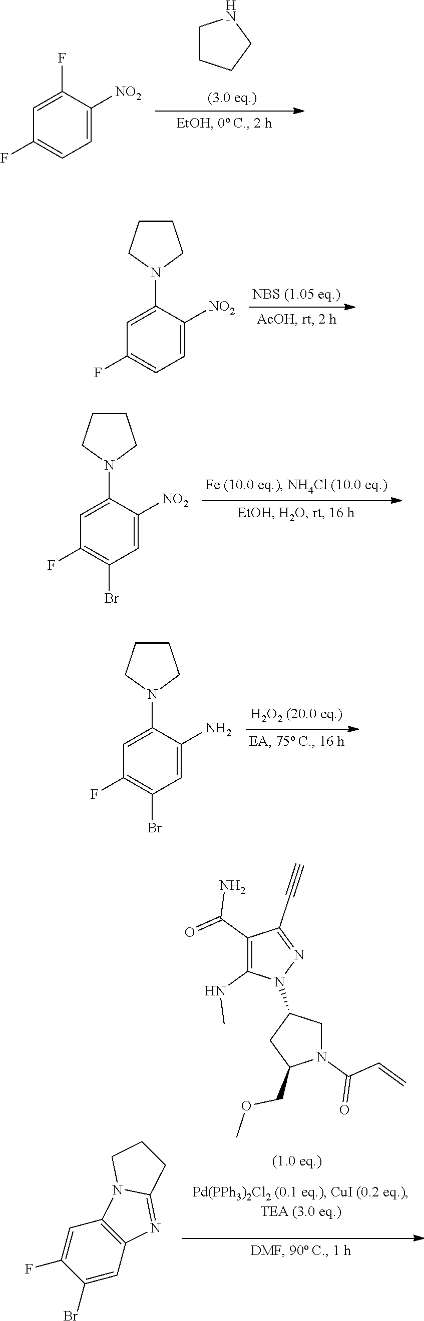

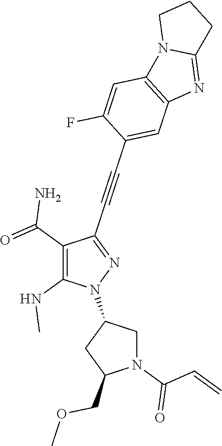 [SMILES: C=CC(=O)N1C[C@@H](n2nc(C#Cc3cc4nc5n(c4cc3F)CCC5)c(C(N)=O)c2NC)C[C@@H]1COC]

1-((3S,5R)-1-acryloyl-5-(methoxymethyl)pyrrolidin-3-yl)-3-((7-fluoro-2,3-dihydro-1H-benzo[d]pyrrolo[1,2-a]imidazol-6-yl)ethynyl)-5-(methylamino)-1H-pyrazole-4-carboxamide. MS ESI calculated for C₂₆H₂₈FN₇O₃[M+H]+, 506.22, found 506.40; ¹H NMR (400 MHz, CDCl₃) δ 7.81 (s, 1H), 7.58-7.47 (m, 2H), 6.78-6.56 (m, 3H), 6.20-6.14 (m, 1H), 5.71-5.67 (m, 1H), 5.32-5.21 (m, 1H), 4.55-4.40 (m, 1H), 4.14-4.11 (m, 2H), 4.06-3.72 (m, 2H), 3.63-3.60 (m, 2H), 3.50-3.45 (m, 3H), 2.96 (t, J=12 Hz, 5H), 2.66-2.60 (m, 2H), 2.46 (d, J=8 Hz, 1H), 2.33-2.29 (in, 1H).

### Example 261: 3-(2-{6-Chloro-3-cyclopropylimidazo[1,2-a]pyridin-7-yl)ethynyl)-1-[(3 S,5R)-5-(methoxymethyl)-1-(prop-2-enoyl)pyrrolidin-3-yl]-5-(methylamino)pyrazole-4-carboxamide

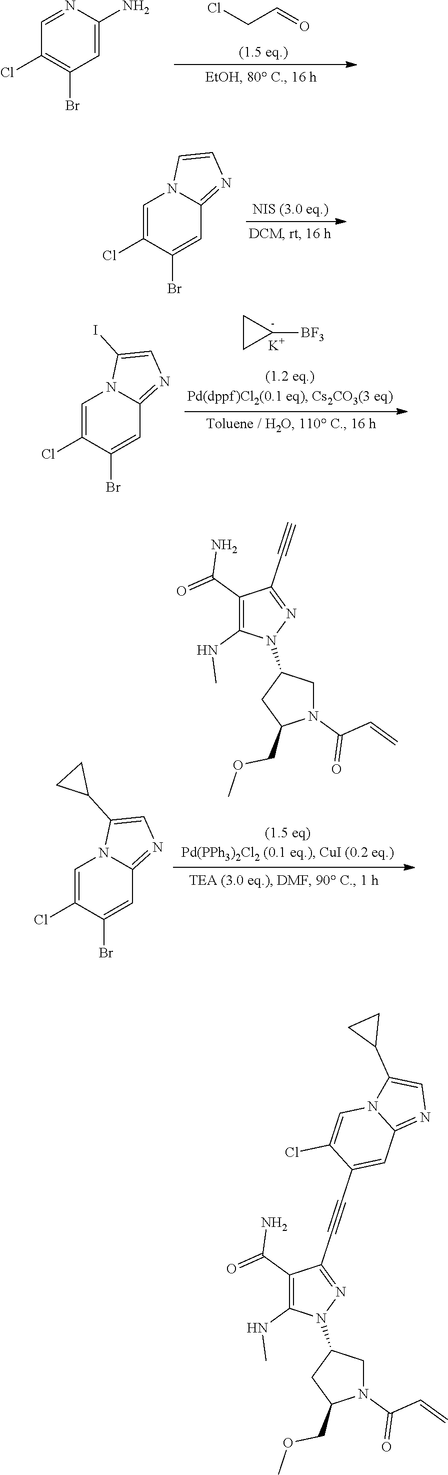

### Step 1: 7-Bromo-6-chloroimidazo[1,2-a]pyridine

A solution of 4-bromo-5-chloropyridin-2-amine (3.00 g, 14.46 mmol) and chloroacetaldehyde (4.26 g, 21.69 mmol) in EtOH (30.00 mL) was stirred for 16 h at 80° C. under nitrogen atmosphere. The resulting mixture was concentrated under reduced pressure. The residue was purified by silica gel column chromatography, eluted with DCM/MeOH (10/1). The fractions contained desired product were combined and concentrated to afford 7-bromo-6-chloroimidazo[1,2-a]pyridine (2.50 g, 74%) as a yellow solid. MS ESI calculated for C₇H₄BrClN₂ [M+H]⁺, 231.48, 233.48, found 231.00, 233.00.

### Step 2: 7-Bromo-6-chloro-3-iodoimidazo[1,2-a]pyridine

To a stirred solution of 7-bromo-6-chloroimidazo[1,2-a]pyridine (2.10 g, 9.07 mmol) in DCM (21.00 mL) was added NIS (6.12 g, 27.21 mmol) in portions at room temperature under nitrogen atmosphere. The reaction mixture was stirred for 16 h at room temperature under nitrogen atmosphere. The resulting mixture was diluted with water (70 mL) and extracted with EA (2×100 mL). The combined organic layers was washed with water (2×100 mL), dried over anhydrous Na₂SO₄. After filtration, the filtrate was concentrated under reduced pressure. The residue was purified by silica gel column chromatography, eluted with DCM/MeOH (5/1). The fractions contained desired product were combined and concentrated to afford 7-bromo-6-chloro-3-iodoimidazo[1,2-a]pyridine (1.80 g, 55%) as an off-white solid. MS ESI calculated for C₇H₃BrClIN₂ [M+H]+, 356.82, 358.82, found 356.90, 358.70.

### Step 3: 7-Bromo-6-chloro-3-cyclopropylimidazo[1,2-a]pyridine

To a stirred solution of 7-bromo-6-chloro-3-iodoimidazo[1,2-a]pyridine (0.76 g, 2.12 mmol) and potassium 1-(trifluoro-lambda4-boranyl)cyclopropan-1-ide (0.37 g, 2.55 mmol) in toluene (7.00 mL) and water (0.70 mL) were added Cs₂CO₃ (2.08 g, 6.38 mmol) and Pd(dppf)Cl₂ (0.15 g, 0.21 mmol). The reaction mixture was irradiated with microwave radiation for 16 h at 110° C. The resulting mixture was concentrated under reduced pressure. The residue was purified by silica gel column chromatography, eluted with PE/EA (5/1). The fractions contained desired product were combined and concentrated to afford 7-bromo-6-chloro-3-cyclopropylimidazo[1,2-a]pyridine (35.00 mg, 6%) as a brown solid. MS ESI calculated for C₁₀H₈BrClN₂ [M+H]⁺, 270.96, 272.95, found 270.90, 272.90.

### Step 4: 3-(2-{6-Chloro-3-cyclopropylimidazo[1,2-a]pyridin-7-yl}ethynyl)-1-[(3S,5R)-5-(methoxymethyl)-1-(prop-2-enoyl)pyrrolidin-3-yl]-5-(methylamino)pyrazole-4-carboxamide

To a stirred solution of 7-bromo-6-chloro-3-cyclopropylimidazo[1,2-a]pyridine (35.00 mg, 0.12 mmol) and 3-ethynyl-1-[(3S,5R)-5-(methoxymethyl)-1-(prop-2-enoyl)pyrrolidin-3-yl]-5-(methylamino)pyrazole-4-carboxamide (64.07 mg, 0.19 mmol) and Pd(PPh₃)₂Cl₂ (9.05 mg, 0.01 mmol) and CuI (4.91 mg, 0.02 mmol) in DMF (0.50 mL) was added TEA (39.13 mg, 0.38 mmol). The reaction mixture was degassed with nitrogen for three times and stirred for 1 h at 90° C. The resulting mixture was concentrated under reduced pressure. The residue was purified by silica gel column chromatography, eluted with DCM/MeOH (5/1). The fractions contained desired product were combined and concentrated to afford the crude product which was further purified by reverse flash chromatography with the following conditions: column: C18 silica gel; mobile phase: ACN in water (10 mmol/L NH₄HCO₃), 10% to 50% gradient in 10 min; detector: UV 254 nm. The fractions contained desired product were combined and concentrated to afford 3-(2-{6-chloro-3-cyclopropylimidazo[1,2-a]pyridin-7-yl}ethynyl)-1-[(3S,5R)-5-(methoxymethyl)-1-(prop-2-enoyl)pyrrolidin-3-yl]-5-(methylamino)pyrazole-4-carboxamide (32.40 mg, 48%) as an off-white solid. MS ESI calculated for C₂₆H₂UClN₇O₃[M+H]⁺, 522.19, found 522.25; ¹H NMR (400 MHz, Chloroform-d) δ 8.90-8.12 (s, 1H), 7.10-6.90 (m, 2H), 6.60-6.44 (m, 2H), 5.80-5.73 (s, 1H), 5.63-5.53 (s, 1H), 5.41-5.31 (s, 1H), 4.67-4.42 (m, 1H), 4.19-3.87 (m, 3H), 3.60-3.44 (m, 1H), 3.40-3.39 (m, 3H), 3.17-3.06 (m, 4H), 2.80-2.72 (m, 1H), 2.49-2.24 (m, 1H), 1.22-1.20 (s, 2H), 0.85-0.78 (s, 2H).

### Example 262: 3-(2-(3-Cyano-6-fluoroimidazo[1,2-a]pyridin-7-yl)ethynyl)-1-[(3S,5R)-5-(methoxymethyl)-1-(prop-2-enoyl)pyrrolidin-3-yl]-5-(methylamino)pyrazole-4-carboxamide

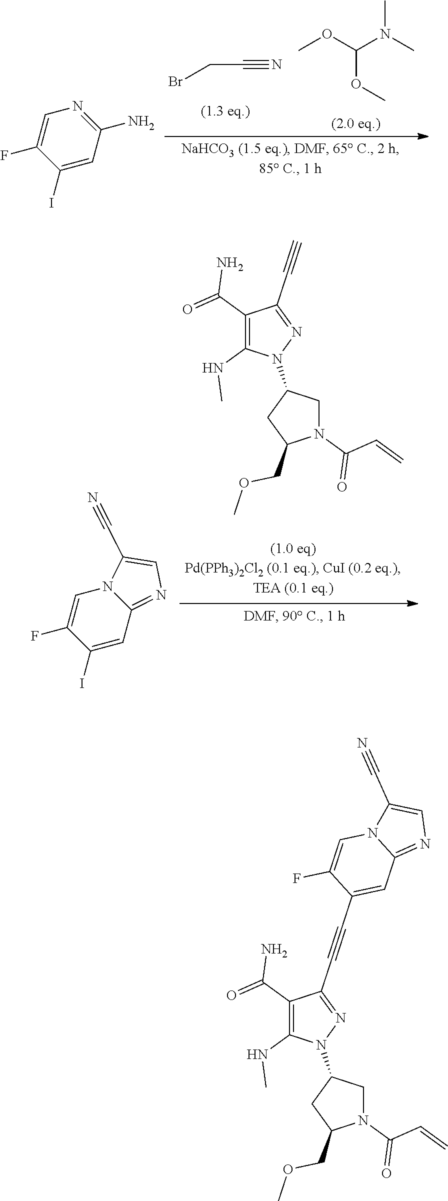

3-(2-{3-cyano-6-fluoroimidazo[1,2-a]pyridin-7-yl}ethynyl)-1-[(3 S,5R)-5-(methoxymethyl)-1-(prop-2-enoyl)pyrrolidin-3-yl]-5-(methylamino)pyrazole-4-carboxamide. MS ESI calculated for C₂₄H₂₃FN₈O₃ [M+H]+, 491.19, found 491.35; ¹H NMR (400 MHz, CDCl₃) δ 9.15 (s, 1H), 8.57 (s, 1H), 8.31-8.29 (m, 1H), 7.52 (s, 1H), 6.65-6.55 (m, 3H), 6.19 (d, J=16.4 Hz, 1H), 5.70 (d, J=2.4 Hz, 1H), 5.28-5.26 (m, 1H), 4.41-4.03 (m, 1H), 3.90-3.58 (m, 2H), 3.60-3.58 (m, 2H), 3.31 (s, 3H), 2.96 (s, 3H), 2.59 (m, 1H), 2.20-2.39 (m, 1H).

### Example 263: 3-(2-{3-Cyanopyrazolo[1,5-a]pyridin-6-yl}ethynyl)-1-[(3S,5R)-5-(methoxymethyl)-1-(prop-2-enoyl)pyrrolidin-3-yl]-5-(methylamino)pyrazole-4-carboxamide

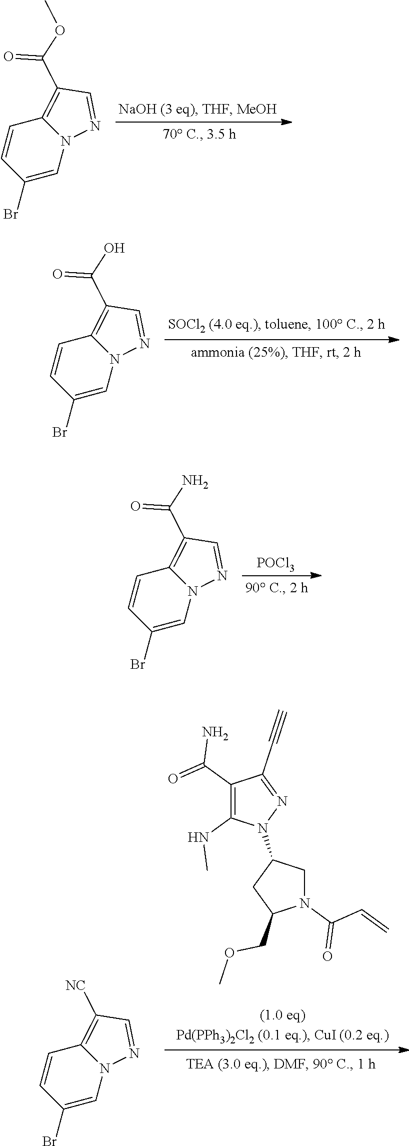

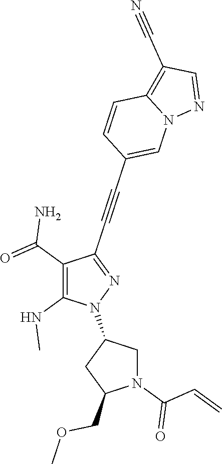 [SMILES: C=CC(=O)N1C[C@@H](n2nc(C#Cc3ccc4c(C#N)cnn4c3)c(C(N)=O)c2NC)C[C@@H]1COC]

3-(2-{3-cyanopyrazolo[1,5-a]pyridin-6-yl}ethynyl)-1-[(3S,5R)-5-(methoxymethyl)-1-(prop-2-enoyl)pyrrolidin-3-yl]-5-(methylamino)pyrazole-4-carboxamide. MS ESI calculated for C₂₄H₂₄N₅O₃ [M+H]+, 473.20, found 473.25; ¹H NMR (400 MHz, DMSO-d₆) δ 9.43 (s, 1H), 8.76 (s, 1H), 8.00 (dd, J=9.1, 1.0 Hz, 1H), 7.73 (dd, J=9.1, 1.5 Hz, 1H), 7.32 (s, 1H), 6.87 (s, 1H), 6.73 (dd, J=16.7, 10.3 Hz, 2H), 6.20-6.14 (m, 1H), 5.76-5.70 (m, 1H), 5.29-5.23 (m, 1H), 4.54 (s, 1H), 4.03-3.60 (m, 2H), 3.42-3.53 (m, 2H), 3.32 (s, 3H), 2.95-2.89 (m, 3H), 2.52-2.45 (m, 1H), 2.32 (s, 1H).

### Example 264: 3-[2-(3,6-Difluoroquinolin-7-yl)ethynyl]-1-[(3S,5R)-5-(methoxymethyl)-1-(prop-2-enoyl)pyrrolidin-3-yl]-5-(methylamino)pyrazole-4-carboxamide

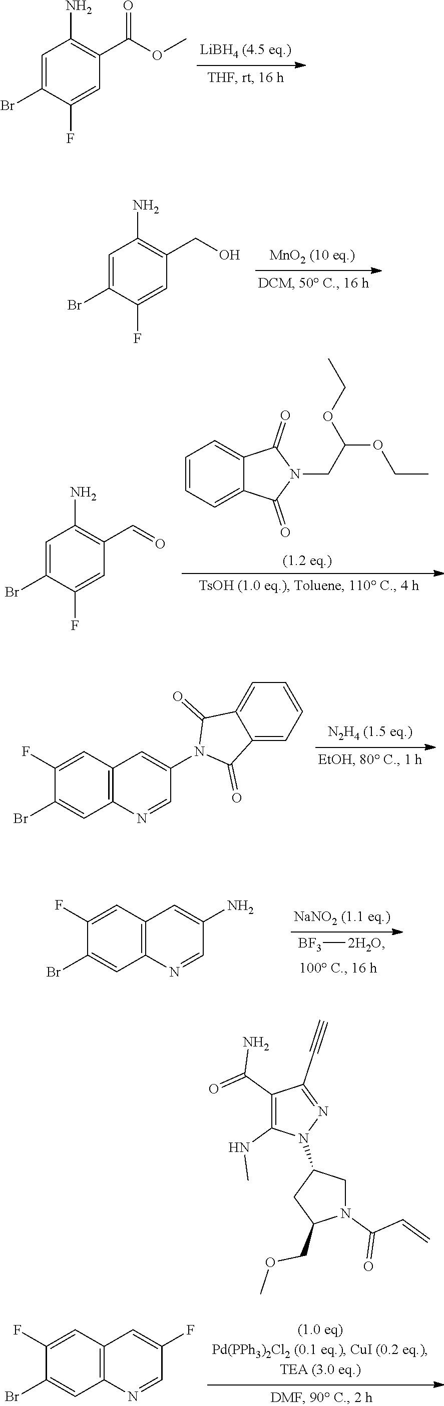

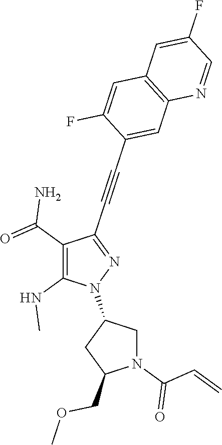 [SMILES: C=CC(=O)N1C[C@@H](n2nc(C#Cc3cc4ncc(F)cc4cc3F)c(C(N)=O)c2NC)C[C@@H]1COC]

3-[2-(3,6-difluoroquinolin-7-yl)ethynyl]-1-[(3S,5R)-5-(methoxymethyl)-1-(prop-2-enoyl)pyrrolidin-3-yl]-5-(methylamino)pyrazole-4-carboxamide. MS ESI calculated for C₂₅H₂₄F₂N₆O₃[M+H]⁺, 495.19, found 495.30. ¹H NMR (400 MHz, Chloroform-d) δ 8.84 (d, J=2.7 Hz, 1H), 8.38 (d, J=6.6 Hz, 1H), 7.75 (d, J=2.7 Hz, 1H), 7.50 (d, J=9.6 Hz, 1H), 6.86 (d, J=6.1 Hz, 2H), 6.49-6.41 (m, 2H), 5.73-5.64 (m, 1H), 5.60-5.37 (m, 2H), 4.59 (d, J=9.0 Hz, 1H), 4.14-4.02 (m, 2H), 3.92-3.62 (m, 1H), 3.50-3.44 (m, 1H), 3.39 (d, J=5.0 Hz, 3H), 3.08 (d, J=5.8 Hz, 3H), 2.79-2.68 (m, 1H), 2.45-2.31 (m, 1H).

### Example 265: 3-[2-(3,6-Dimethylquinolin-7-yl)ethynyl]-1-[(3S,5R)-5-(methoxymethyl)-1-(prop-2-enoyl)pyrrolidin-3-yl]-5-(methylamino)pyrazole-4-carboxamide

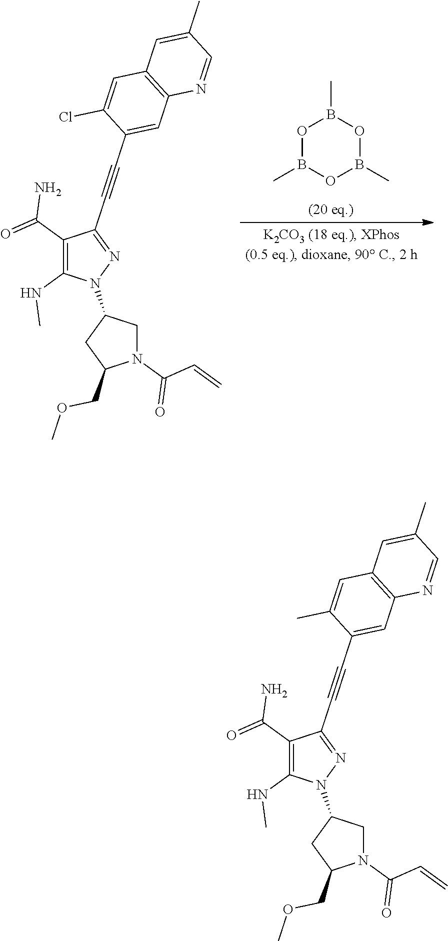

3-[2-(3,6-dimethylquinolin-7-yl)ethynyl]-1-[(3S,5R)-5-(methoxymethyl)-1-(prop-2-enoyl)pyrrolidin-3-yl]-5-(methylamino)pyrazole-4-carboxamide. MS ESI calculated for C₂₇H₃₀N₆O₃ [M+H]⁺, 487.24, found 487.30; ¹H NMR (400 MHz, Chloroform-d) δ 8.76 (d, J=2.2 Hz, 1H), 8.28 (d, J=3.7 Hz, 1H), 7.86 (s, 1H), 7.60 (s, 1H), 7.03-6.72 (m, 2H), 6.60-6.43 (m, 2H), 5.78-5.70 (m, 1H), 5.57-5.46 (m, 1H), 5.35 (d, J=27.2 Hz, 1H), 4.67-4.42 (m, 1H), 4.13-4.10 (m, 1H), 4.08-3.98 (m, 1H), 3.92-3.58 (m, 1H), 3.50-3.42 (m, 1H), 3.39 (d, J=4.6 Hz, 3H), 3.07 (d, J=5.8 Hz, 3H), 2.80-2.69 (m, 1H), 2.67 (d, J=3.7 Hz, 3H), 2.54 (s, 3H), 2.33-2.28 (m, 1H).

### Example 266: 1-((3S,5R)-1-Acryloyl-5-(methoxymethyl)pyrrolidin-3-yl)-3-((6-fluoro-4-methylcinnolin-7-yl)ethynyl)-5-(methylamino)-1H-pyrazole-4-carboxamide

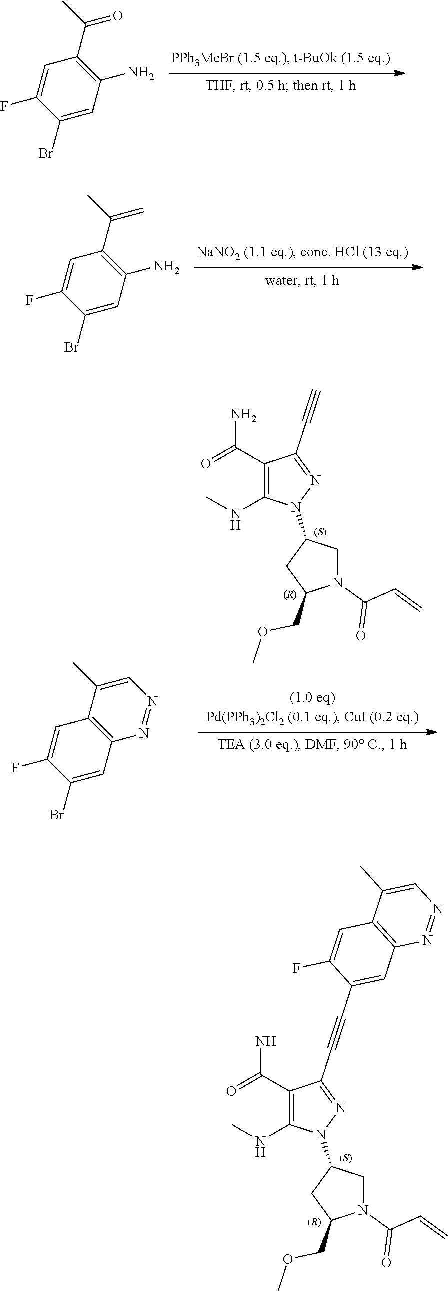

1-((3S,5R)-1-acryloyl-5-(methoxymethyl)pyrrolidin-3-yl)-3-((6-fluoro-4-methylcinnolin-7-yl)ethynyl)-5-(methylamino)-1H-pyrazole-4-carboxamide. MS ESI calculated for C₂₅H₂₆FN₇O₃ [M+H]⁺, 492.21, found 492.35; ¹H NMR (400 MHz, DMSO-d₆) S 9.28 (s, 1H), 8.82 (dd, J=7.0, 2.6 Hz, 1H), 8.12 (d, J=10.3 Hz, 1H), 7.49 (s, 1H), 6.90 (s, 1H), 6.79-6.54 (m, 2H), 6.20-6.15 (m, 1H), 5.72-5.69 (m, 1H), 5.32-5.23 (m, 1H), 4.65-4.33 (m, 1H), 4.11-3.70 (m, 2H), 3.61 (dd, J=9.4, 5.2 Hz, 1H), 3.55-3.41 (m, 2H), 3.30 (s, 3H), 2.96 (t, J=5.1 Hz, 3H), 2.66 (s, 3H), 2.38-2.27 (m, 1H).

### Example 267: 3-[2-(6-Chloro-3-fluoroquinolin-7-yl)ethynyl]-1-[(3S,5R)-5-(methoxymethyl)-1-(prop-2-enoyl)pyrrolidin-3-yl]-5-(methylamino)pyrazole-4-carboxamide

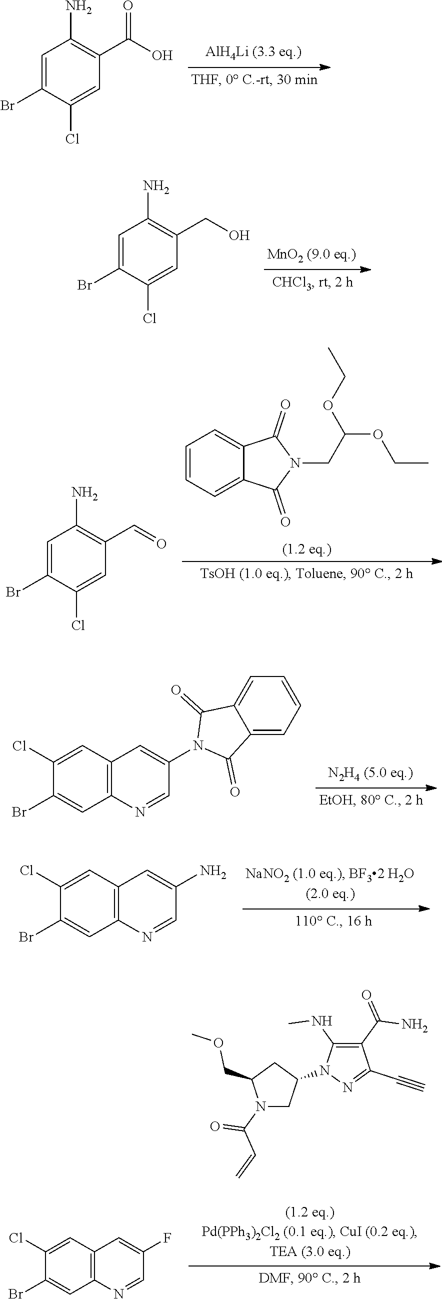

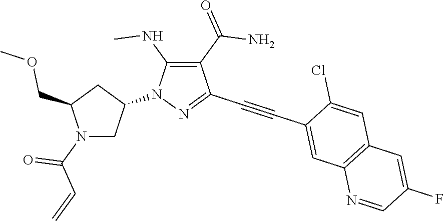 [SMILES: C=CC(=O)N1C[C@@H](n2nc(C#Cc3cc4ncc(F)cc4cc3Cl)c(C(N)=O)c2NC)C[C@@H]1COC]

3-[2-(6-chloro-3-fluoroquinolin-7-yl)ethynyl]-1-[(3S,5R)-5-(methoxymethyl)-1-(prop-2-enoyl)pyrrolidin-3-yl]-5-(methylamino)pyrazole-4-carboxamide. MS ESI calculated for C₂₅H₂₄ClFN₆O₃[M+H]⁺, 511.16, found 511.20; ¹H NMR (400 MHz, Chloroform-d) δ 8.84 (d, J=2.8 Hz, 1H), 8.41 (d, J=6.3 Hz, 1H), 7.87 (d, J=2.1 Hz, 1H), 7.69-7.56 (m, 1H), 7.06 (s, 1H), 6.83 (d, J=6.1 Hz, 1H), 6.54-6.40 (m, 1H), 6.44-6.35 (m, 1H), 5.76-5.67 (m, 1H), 5.57-5.51 (m, 1H), 5.40 (s, 1H), 4.57 (d, J=9.0 Hz, 1H), 4.16-3.97 (m, 2H), 4.00-3.87 (m, 1H), 3.54-3.41 (m, 1H), 3.37 (d, J=5.2 Hz, 3H), 3.04-2.91 (m, 3H), 2.71-2.65 (m, 1H), 2.35-2.28 (m, 1H).

### Example 268: 1-((3S,5R)-1-acryloyl-5-(methoxymethyl)pyrrolidin-3-yl)-3-((6-chloro-4-methylcinnolin-7-yl)ethynyl)-5-(methylamino)-1H-pyrazole-4-carboxamide

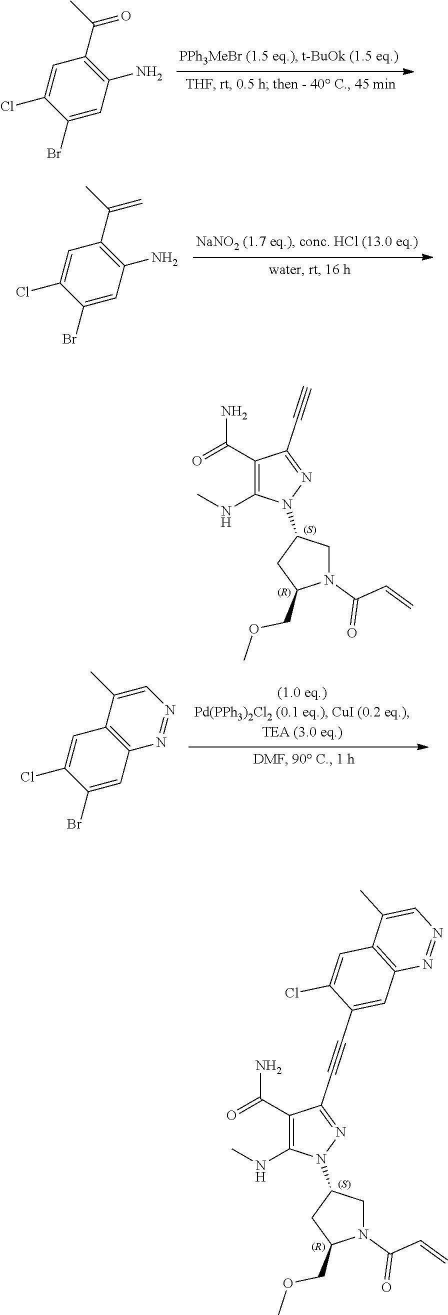

### Step 1: 5-Bromo-4-chloro-2-(prop-1-en-2-yl)aniline

To a stirred mixture of methyltriphenylphosphanium bromide (2.16 g, 6.04 mmol) in THF (10.00 mL) was added t-BuOK (0.68 g, 6.04 mmol) in portions at 0° C. under nitrogen atmosphere. After stirred for 30 min at room temperature, 1-(2-amino-4-bromo-5-chlorophenyl)ethanone (1.00 g, 4.02 mmol) in THF (5.00 mL) was added dropwise at 0° C. under nitrogen atmosphere. The reaction mixture was stirred for 45 min at −40° C. The resulting mixture was quenched with water at −40° C. and extracted with EA (3×80 mL). The combined organic layers was washed with brine (60 mL), dried over anhydrous Na₂SO₄, filtered and concentrated under reduced pressure. The residue was purified by silica gel column chromatography, eluted with PE/DCM (4/1). The fractions contained desired product were combined and concentrated to afford 5-bromo-4-chloro-2-(prop-1-en-2-yl)aniline (0.10 g, 10%) as a yellow oil. MS ESI calculated for C9H₉BrClN [M+H]+, 245.96, 247.96, found 246.05, 248.05.

### Step 2: 7-Bromo-6-chloro-4-methylcinnoline

To a stirred mixture of 5-bromo-4-chloro-2-(prop-1-en-2-yl)aniline (0.10 g, 0.43 mmol) and conc.HCl (0.46 mL, 5.54 mmol) in water (0.46 mL) was added NaNO2 (0.36 mL, 2 M) dropwise at room temperature. The reaction mixture was stirred for 16 h at room temperature. The resulting mixture was diluted with water (10 mL) and extracted with EA (3×30 mL). The combined organic layers was washed with brine (2×30 mL), dried over anhydrous Na2SO4, filtered and concentrated under reduced pressure. The residue was purified by silica gel column chromatography, eluted with PE/EA (1/1). The fractions contained desired product were combined and concentrated to afford 7-bromo-6-chloro-4-methylcinnoline (90.00 mg, 82%) as a yellow solid. MS ESI calculated for C9H6BrClN2 [M+H]+, 256.94, 258.94, found 257.00, 259.00; 1H NMR (400 MHz, CDCl3) δ 9.19 (s, 1H), 8.86 (s, 1H), 8.12 (s, 1H), 2.68 (s, 3H).

### Step 3: 1-((3S,5R)-1-acryloyl-5-(methoxymethyl)pyrrolidin-3-yl)-3-((6-chloro-4-methylcinnolin-7-yl)ethynyl)-5-(methylamino)-1H-pyrazole-4-carboxamide

To a stirred mixture of 7-bromo-6-chloro-4-methylcinnoline (80.00 mg, 0.31 mmol), 3-ethynyl-1-[(3S,5R)-5-(methoxymethyl)-1-(prop-2-enoyl)pyrrolidin-3-yl]-5-(methylamino)pyrazole-4-carboxamide (0.10 g, 0.31 mmol), Pd(PPh3)2Cl2 (21.80 mg, 0.03 mmol) and CuI (11.83 mg, 0.06 mmol) in DMF (1.00 mL) was added TEA (94.31 mg, 0.93 mmol) at room temperature. The reaction mixture was degassed with argon for three times and stirred for 1 h at 90° C. The resulting mixture was concentrated under reduced pressure. The residue was purified by silica gel column chromatography, eluted with CH2Cl2/MeOH (10/1) to afford the crude product. The crude product (120 mg) was purified by Prep-HPLC with the following conditions: Column: XBridge Prep OBD C18 Column, 30×150 mm, 5 Om; Mobile Phase A: water (10 mmol/L NH4HCO3), Mobile Phase B: ACN; Flow rate: 60 mL/min; Gradient: 25% B to 35% B in 8 min; Wave length: 254 nm; RT: 8 min. The fractions contained desired product were combined and concentrated to afford 1-((3S,5R)-1-acryloyl-5-(methoxymethyl)pyrrolidin-3-yl)-3-((6-chloro-4-methylcinnolin-7-yl)ethynyl)-5-(methylamino)-1H-pyrazole-4-carboxamide (76.50 mg, 48%) as a white solid. MS ESI calculated for C25H26ClN7O3 [M+H]+, 508.18, found 508.20; 1H NMR (400 MHz, DMSO-d6) δ 9.32 (s, 1H), 8.81 (s, 1H), 8.46 (s, 1H), 7.50 (s, 1H), 6.94 (s, 1H), 6.78-6.50 (m, 2H), 6.23-6.15 (m, 1H), 5.74-5.67 (m, 1H), 5.30-5.24 (m, 1H), 4.63-4.33 (m, 1H), 4.05 (dd, J=10.6, 7.4 Hz, 1H), 3.97-3.83 (m, 1H), 3.81-3.57 (m, 1H), 3.47 (dd, J=9.6, 4.0 Hz, 1H), 3.32 (s, 3H), 2.96 (t, J=5.1 Hz, 3H), 2.69 (s, 3H), 2.62 (t, J=10.5 Hz, 1H), 2.33 (d, J=4.5 Hz, 1H).

### Example 269: 3-(2-{3-Cyanoimidazo[1,2-a]pyridin-7-yl}ethynyl)-1-[(3S,5R)-5-(methoxymethyl)-1-(prop-2-enoyl)pyrrolidin-3-yl]-5-(methylamino)pyrazole-4-carboxamide

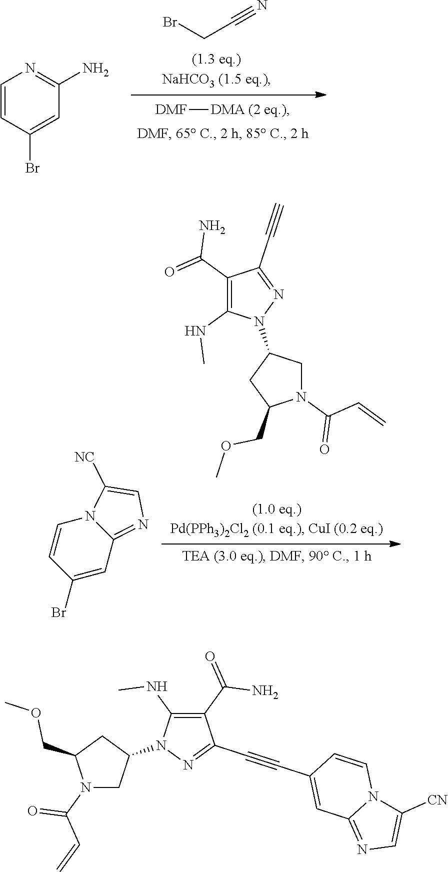

3-(2-{3-cyanoimidazo[1,2-a]pyridin-7-yl}ethynyl)-1-[(3S,5R)-5-(methoxymethyl)-1-(prop-2-enoyl)pyrrolidin-3-yl]-5-(methylamino)pyrazole-4-carboxamide. MS ESI calculated for C₂₄H₂₄N₅O₃ [M+H]⁺, 473.20, found 473.05; ¹H NMR (400 MHz, CDCl₃) δ 8.50 (s, 1H), 8.01 (s, 1H), 7.23 (d, J=6.9 Hz, 1H), 6.77 (d, J=47.9 Hz, 1H), 6.64-6.37 (m, 2H), 5.75-5.71 (m, 1H), 5.57-5.28 (m, 2H), 4.62-4.40 (m, 1H), 4.09-4.04 (m, 2H), 3.93-3.90 (m, 1H), 3.56-3.43 (m, 1H), 3.39 (d, J=5.3 Hz, 3H), 3.12-3.03 (m, 3H), 2.76-2.64 (m, 1H), 2.44-2.32 (m, 1H).

### Example 270: 1-((3S,5R)-1-acryloyl-5-(methoxymethyl)pyrrolidin-3-yl)-3-((3-cyano-4-methoxypyrazolo[1,5-a]pyridin-6-yl)ethynyl)-5-(methylamino)-1H-pyrazole-4-carboxamide

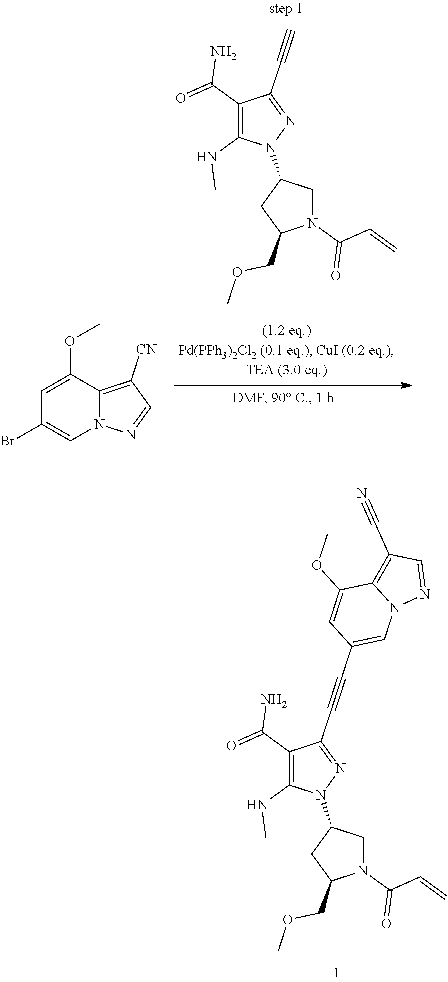 [SMILES: C#Cc1nn([C@H]2C[C@H](COC)N(C(=O)C=C)C2)c(NC)c1C(N)=O ; COc1cc(Br)cn2ncc(C#N)c12 ; C=CC(=O)N1C[C@@H](n2nc(C#Cc3cc(OC)c4c(C#N)cnn4c3)c(C(N)=O)c2NC)C[C@@H]1COC]

1-((3S,5R)-1-acryloyl-5-(methoxymethyl)pyrrolidin-3-yl)-3-((3-cyano-4-methoxypyrazolo[1,5-a]pyridin-6-yl)ethynyl)-5-(methylamino)-1H-pyrazole-4-carboxamide. MS ESI calculated for C₂₂H₂₉FN₄O₅[M+H]⁺, 503.21, found 503.35; ¹H NMR (400 MHz, Chloroform-d) δ 8.43 (d, J=1.1 Hz, 1H), 8.24 (s, 1H), 6.83 (s, 1H), 6.75 (d, J=1.1 Hz, 1H), 6.56-6.37 (m, 2H), 5.73 (dd, J=7.5, 4.8 Hz, 1H), 5.52 (m, 2H), 4.59 (d, J=8.9 Hz, 1H), 4.07 (s, 5H), 4.02-3.87 (m, 1H), 3.55-3.33 (m, 4H), 3.09 (d, J=5.7 Hz, 3H), 2.69 (m, 1H), 2.37 (m, 1H).

### Example 271: 3-(2-{1-[(1S)-2,2-Difluorocyclopropyl]-6-fluoro-1,3-benzodiazol-5-yl}ethynyl)-1-[(3S,5R)-5-(methoxymethyl)-1-(prop-2-enoyl)pyrrolidin-3-yl]-5-(methylamino)pyrazole-4-carboxamide

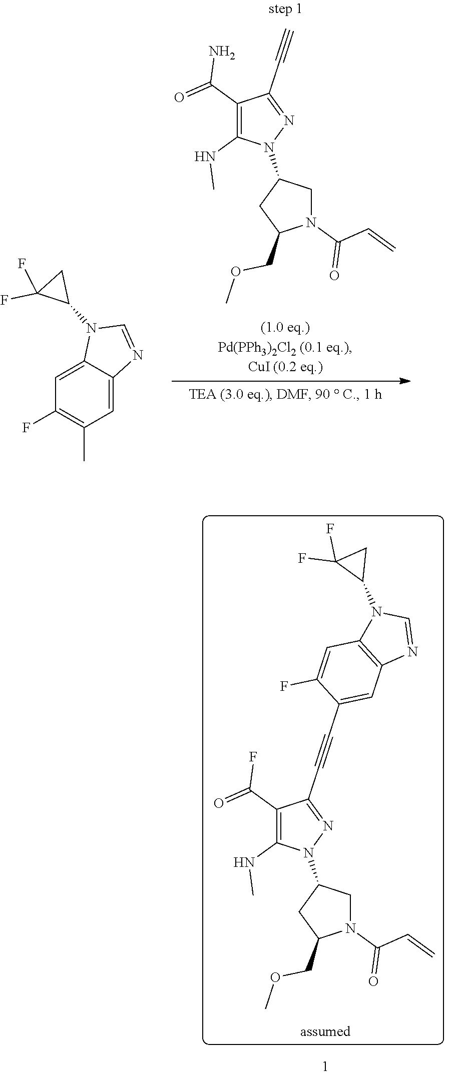 [SMILES: Cc1cc2ncn([C@H]3CC3(F)F)c2cc1F ; C#Cc1nn([C@H]2C[C@H](COC)N(C(=O)C=C)C2)c(NC)c1C(N)=O ; C=CC(=O)N1C[C@@H](n2nc(C#Cc3cc4ncn([C@H]5CC5(F)F)c4cc3F)c(C(=O)F)c2NC)C[C@@H]1COC]

To a stirred mixture of (S)-1-(2,2-difluorocyclopropyl)-6-fluoro-5-iodo-1H-benzo[d]imidazole (80 mg, 0.23 mmol) and 3-ethynyl-1-[(3S,5R)-5-(methoxymethyl)-1-(prop-2-enoyl)pyrrolidin-3-yl]-5-(methylamino)pyrazole-4-carboxamide (78.42 mg, 0.23 mmol) in DMF (0.50 mL) were added Pd(PPh₃)₂Cl₂ (16.61 mg, 0.02 mmol), CuI (9.01 mg, 0.04 mmol) and TEA (71.84 mg, 0.71 mmol). The reaction mixture was degassed with argon for three times and stirred for 1 h at 90 C. The resulting mixture was concentrated under reduced pressure. The residue was purified by silica gel column chromatography, eluted with CH₂Cl₂/MeOH (10/1) to afford the crude product which was further purified by Prep-HPLC with the following conditions: Column: XBridge Prep OBD C18 Column, 30×150 mm, 5 μm; Mobile Phase A: water (10 mmol/L NH₄HCO₃), Mobile Phase B: ACN; Flow rate: 60 mL/min; Gradient: 28% B to 38% B in 8 min, 38% B; Wave Length: 254 nm; RT1: 7.17 min. The fractions contained desired product were combined and concentrated to afford 3-(2-{1-[(1S)-2,2-difluorocyclopropyl]-6-fluoro-1,3-benzodiazol-5-yl}ethynyl)-1-[(3S,5R)-5-(methoxymethyl)-1-(prop-2-enoyl)pyrrolidin-3-yl]-5-(methylamino)pyrazole-4-carboxamide (stereochemistry arbitrarily assigned) (71.20 mg, 55%) as an off-white solid. MS ESI calculated for C₂₆H₂₆F₃N₇O₃[M+H]⁺, 542.50, found 542.55; ¹H NMR (400 MHz, CDCl₃) δ 8.04 (s, 2H), 7.26 (d, J=10.0 Hz, 2H), 6.59-6.36 (m, 2H), 5.77-5.68 (m, 1H), 5.59-5.46 (m, 1H), 5.41-5.28 (i, 1H), 4.58 (d, J=8.9 Hz, 1H), 4.08-4.02 (m, 2H), 3.97-3.92 (m, 2H), 3.55-3.42 (m, 1H), 3.39 (d, J=4.5 Hz, 3H), 3.05 (d, J=15.2 Hz, 3H), 2.79-2.66 (m, 1H), 2.44-2.25 (m, 2H), 2.09-2.04 (m, 1H).

### Example 272: 3-{2-[1-((R)-2,2-difluorocyclopropyl)-6-fluoro-1,3-benzodiazol-5-yl]ethynyl}-1-[(3 S,5R)-5-(methoxymethyl)-1-(prop-2-enoyl)pyrrolidin-3-yl]-5-(methylamino)pyrazole-4-carboxamide

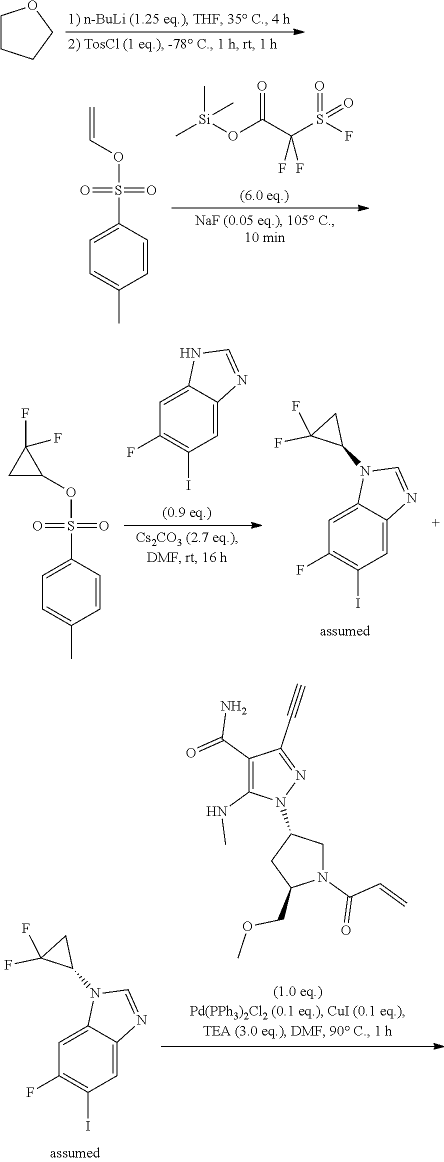

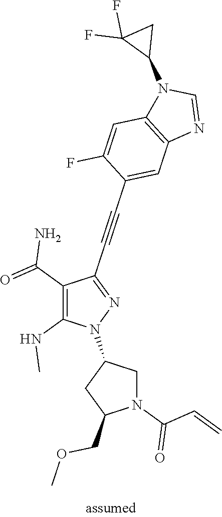 [SMILES: C=CC(=O)N1C[C@@H](n2nc(C#Cc3cc4ncn([C@@H]5CC5(F)F)c4cc3F)c(C(N)=O)c2NC)C[C@@H]1COC]

### Step 1: Vinyl 4-methylbenzenesulfonate

A solution of n-BuLi (13.00 mL, 32.77 mmol) in THF (40.00 mL) was stirred for 4 h at 35° C. under nitrogen atmosphere. Then to the above mixture was added a solution of P-toluenesulfonyl chloride (5.00 g, 26.23 mmol) in THF (13.00 mL) dropwise over 30 min at −78° C. The reaction mixture was stirred for 1 h at −78° C. and another 1 h at room temperature. The resulting mixture was quenched by addition of water (50 mL) and extracted with MTBE (3×50 mL). The combined organic layers was dried over Na₂SO₄ and filtered. The filtrate was concentrated under reduced pressure. The residue was purified by silica gel column chromatography, eluted with PE. The fractions contained desired product were combined and concentrated to afford vinyl 4-methylbenzenesulfonate (3.40 g, 65%) as a colorless oil. MS ESI calculated for C₉H₁₀O₃S [M−H]⁻, 197.04, found 196.90; ¹H NMR (400 MHz, CDCl₃) δ 7.89-7.74 (m, 2H), 7.43-7.33 (m, 2H), 6.62 (dd, J=13.6, 5.9 Hz, 1H), 4.90 (dd, J=13.5, 2.5 Hz, 1H), 4.70 (dd, J=5.9, 2.5 Hz, 1H).

### Step 2: 2,2-Difluorocyclopropyl 4-methylbenzenesulfonate

To a stirred mixture of NaF (59.31 mg, 1.41 mmol) and vinyl 4-methylbenzenesulfonate (2.80 g, 14.12 mmol) was added trimethylsilyl 2,2-difluoro-2-sulfoacetate (21.21 g, 84.74 mmol) dropwise at room temperature under nitrogen atmosphere. The reaction mixture was stirred for 10 min at 105° C. under nitrogen atmosphere. The resulting mixture was purified by silica gel column chromatography, eluted with PE/EA (12/1). The fractions contained desired product were combined and concentrated to afford 2,2-difluorocyclopropyl 4-methylbenzenesulfonate (1.70 g, 48%) as a light yellow solid; ¹H NMR (400 MHz, CDCl₃) δ 7.89-7.80 (m, 2H), 7.45-7.37 (m, 2H), 4.30-4.24 (m, 1H), 2.50 (s, 3H), 1.82-1.58 (m, 2H).

### Step 3: 1-(2,2-Difluorocyclopropyl)-6-fluoro-5-iodo-1,3-benzodiazole & 1-(2,2-difluorocyclopropyl)-5-fluoro-6-iodo-1,3-benzodiazole

To a stirred solution of 2,2-difluorocyclopropyl 4-methylbenzenesulfonate (0.42 g, 1.68 mmol) and Cs₂CO₃ (1.49 g, 4.58 mmol) in DMF (5.00 mL) was added 5-fluoro-6-iodo-3H-1,3-benzodiazole (0.40 g, 1.53 mmol) at room temperature. The reaction mixture was stirred for 16 h at room temperature. The resulting mixture was filtered, the filter cake was washed with DMF (2×20 mL). The filtrate was concentrated under reduced pressure. The residue was purified by reverse flash chromatography with the following conditions: column: C18 silica gel; mobile phase, ACN in water (NH₄HCO₃, 10 mmol/L), 10% to 50% gradient in 40 min; detector: UV 254 nm to afford the crude product (360 mg) which was further purified by Prep-HPLC with the following conditions: Column: CHIRALPAK ID, 2×25 cm, 5 μm; Mobile Phase A: Hex (0.5% 2 M NH₃-MeOH)-HPLC, Mobile Phase B: MeOH:EtOH=1:1-HPLC; Flow rate: 20 mL/min; Gradient: 20% B to 20% B in 15 min; Wave Length: 220/254 nm; RT1: 7.08 min; RT2: 11.51 min; Sample Solvent: MeOH:DCM=1:1. The faster peak (RT1: 7.08 min) was combined and concentrated to afford 1-[(1R)-2,2-difluorocyclopropyl]-6-fluoro-5-iodo-1,3-benzodiazole (80.00 mg, 15%) as a light yellow solid. MS ESI calculated for C₁₀H₆F₃IN₂ [M+H]⁺, 338.85, found 338.85; ¹H NMR (400 MHz, CDCl₃) δ 8.20 (d, J=5.5 Hz, 1H), 7.95 (d, J=1.7 Hz, 1H), 7.23 (d, J=7.3 Hz, 1H), 3.95-3.88 (m, 1H), 2.34-2.25 (m, 1H), 2.09-1.96 (m, 1H). The slower peak (RT1: 11.51 min) was combined and concentrated to afford 1-[(1S)-2,2-difluorocyclopropyl]-6-fluoro-5-iodo-1,3-benzodiazole (90.00 mg, 17%) as a light yellow solid. MS ESI calculated for C₁₀H₆F₃IN₂ [M+H]⁺, 338.85, found 338.85; ¹H NMR (400 MHz, CDCl₃) δ 8.21 (d, J=5.5 Hz, 1H), 7.97 (t, J=1.7 Hz, 1H), 7.23 (d, J=7.3 Hz, 1H), 3.95-3.88 (m, 1H), 2.34-3.25 (m, 1H), 2.07-1.99 (m, 1H).

### Step 4: 3-{2-[1-((R)-2,2-difluorocyclopropyl)-6-fluoro-1,3-benzodiazol-5-yl]ethynyl}-1-[(3S,5R)-5-(methoxymethyl)-1-(prop-2-enoyl)pyrrolidin-3-yl]-5-(methylamino)pyrazole-4-carboxamide

To a stirred mixture of 1-[(1R)-2,2-difluorocyclopropyl]-6-fluoro-5-iodo-1,3-benzodiazole (80.00 mg, 0.24 mmol), 3-ethynyl-1-[(3S,5R)-5-(methoxymethyl)-1-(prop-2-enoyl)pyrrolidin-3-yl]-5-(methylamino)pyrazole-4-carboxamide (78.42 mg, 0.24 mmol), Pd(PPh₃)₂Cl₂ (16.61 mg, 0.02 mmol) and CuI (4.51 mg, 0.02 mmol) in DMF (2.00 mL) was added TEA (71.84 mg, 0.71 mmol) dropwise at room temperature. The reaction mixture was degassed with nitrogen for three times and stirred for 1 h at 90° C. The resulting mixture was concentrated under reduced pressure. The residue was purified by silica gel column chromatography, eluted with CH₂Cl₂/MeOH (10/1) to afford the crude product (100 mg) which was further purified by reverse flash chromatography with the following conditions: column, C18 silica gel; mobile phase: ACN in water (10 mmol/L NH₄HCO₃), 5% to 70% gradient in 40 min; detector: UV 254 nm. The fractions contained desired product were combined and concentrated to afford 3-{2-[1-((R)-(2,2-difluorocyclopropyl)-6-fluoro-1,3-benzodiazol-5-yl]ethynyl}-1-[(3S,5R)-5-(methoxymethyl)-1-(prop-2-enoyl)pyrrolidin-3-yl]-5-(methylamino)pyrazole-4-carboxamide (stereochemistry arbitrarily assigned) (45.30 mg, 35%) as an off-white solid. MS ESI calculated for C₂₆H₂₆F₃N₇O₃[M+H]⁺, 542.21, found 542.30; ¹H NMR (400 MHz, CDCl₃) δ 8.04 (s, 2H), 7.29 (s, 1H), 7.09 (s, 1H), 6.86-6.67 (m, 1H), 6.57-6.37 (m, 2H), 5.72 (dd, J=8.5, 4.0 Hz, 1H), 5.59-5.08 (m, 1H), 4.61-4.36 (m, 1H), 4.09-4.01 (m, 2H), 3.96-3.90 (m, 2H), 3.55-3.42 (m, 1H), 3.39 (d, J=4.4 Hz, 3H), 3.05 (dd, J=15.4, 5.8 Hz, 3H), 2.76-2.71 (m, 1H), 2.32 (dd, J=12.4, 7.0 Hz, 2H), 2.05 (d, J=14.0 Hz, 1H).

### Example 273: 3-{2-[3-(Difluoromethyl)-6-fluoroquinolin-7-yl]ethynyl}-1-[(3S,5R)-5-(methoxymethyl)-1-(prop-2-enoyl)pyrrolidin-3-yl]-5-(methylamino)pyrazole-4-carboxamide

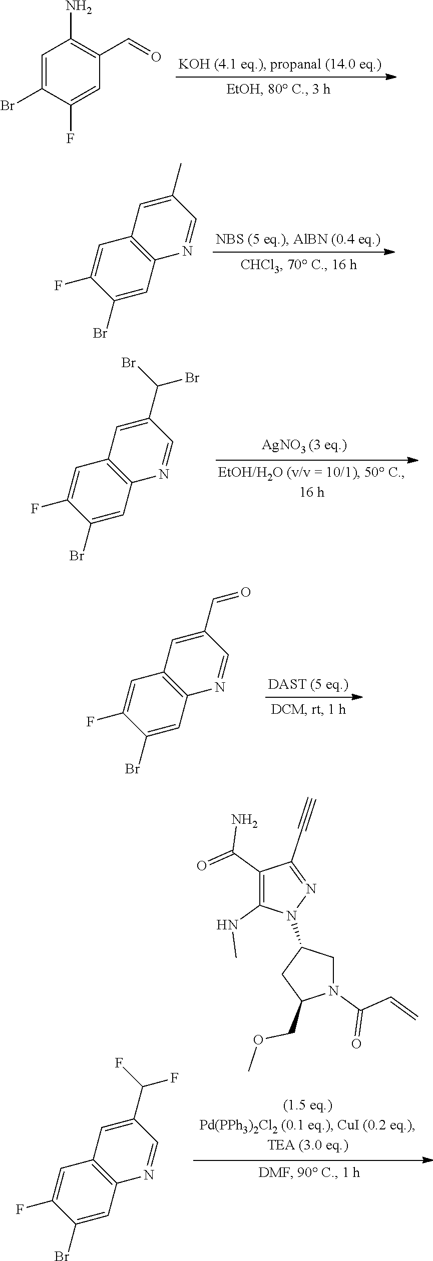

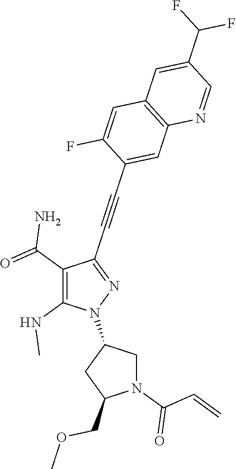 [SMILES: C=CC(=O)N1C[C@@H](n2nc(C#Cc3cc4ncc(C(F)F)cc4cc3F)c(C(N)=O)c2NC)C[C@@H]1COC]

3-{2-[3-(difluoromethyl)-6-fluoroquinolin-7-yl]ethynyl}-1-[(3S,5R)-5-(methoxymethyl)-1-(prop-2-enoyl)pyrrolidin-3-yl]-5-(methylamino)pyrazole-4-carboxamide. MS ESI calculated for C₂₆H₂₅F₃N₆O₃[M+H]⁺, 527.19, found 527.35; ¹H NMR (400 MHz, Chloroform-d) δ 9.08-9.04 (m, 1H), 8.43 (d, J=6.4 Hz, 1H), 8.27 (s, 1H), 7.60 (d, J=9.2 Hz, 1H), 7.02 (d, J=23.0 Hz, 1H), 6.94-6.78 (m, 2H), 6.52-6.34 (m, 2H), 5.74-5.65 (m, 1H), 5.54-5.51 (m, 1H), 5.48-5.29 (m, 1H), 4.65-4.41 (m, 1H), 4.17-3.98 (m, 2H), 3.93-3.54 (m, 1H), 3.47-3.44 (m, 1H), 3.40 (di, J=5.1 Hz, 3H), 3.07-2.84 (m, 3H), 2.78-2.67 (m, 1H), 2.34-2.29 (m, 1H).

### Example 274: 3-[2-(6-Fluoro-4-methylquinolin-7-yl)ethynyl]-1-[(3S,5R)-5-(methoxymethyl)-1-(prop-2-enoyl)pyrrolidin-3-yl]-5-(methylamino)pyrazole-4-carboxamide

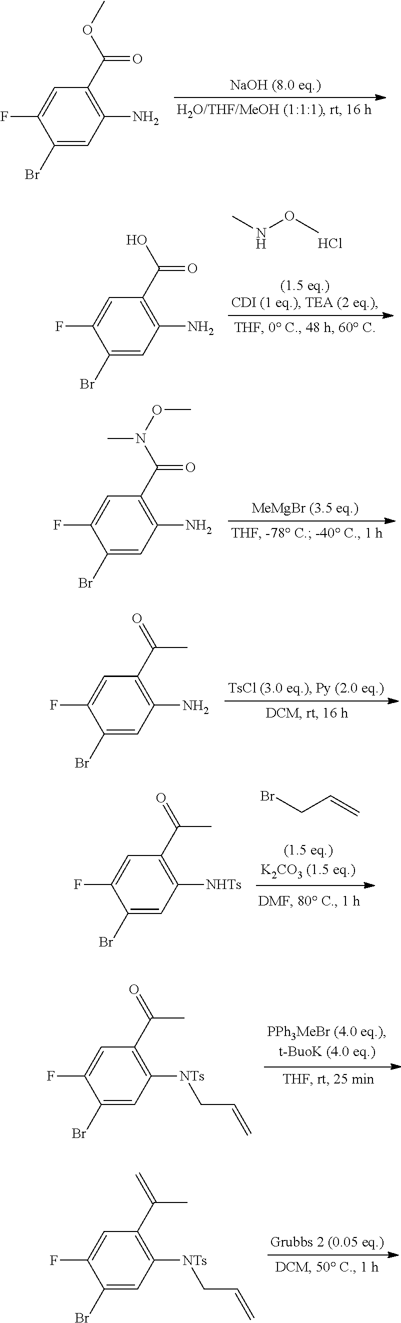

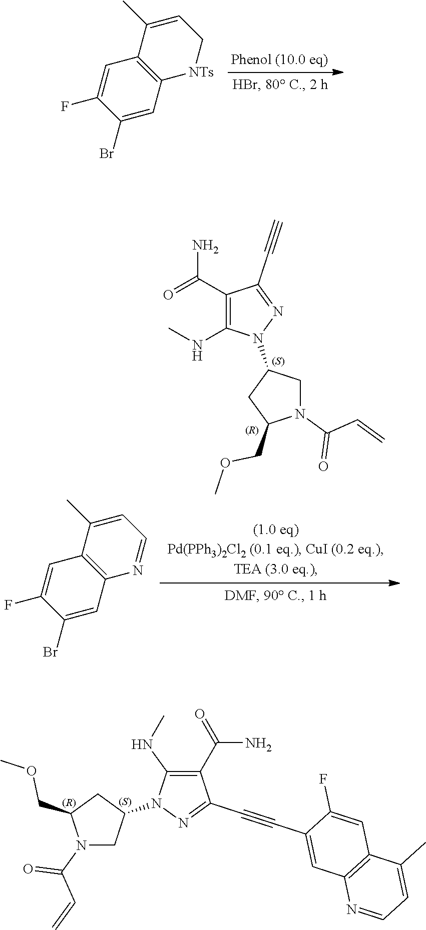

3-[2-(6-fluoro-4-methylquinolin-7-yl)ethynyl]-1-[(3S,5R)-5-(methoxymethyl)-1-(prop-2-enoyl)pyrrolidin-3-yl]-5-(methylamino)pyrazole-4-carboxamide. MS ESI calculated for C₂₆H₂₇FN₆O₃[M+H]+, 491.21, found 491.25; ¹H NMR (400 MHz, DMSO-d₆) δ 8.81 (d, J=4.4 Hz, 1H), 8.33 (d, J=6.3 Hz, 1H), 8.01 (di, J=10.9 Hz, 1H), 7.47 (d, J=4.4 Hz, 2H), 6.86 (s, 1H), 6.77-6.53 (m, 2H), 6.17 (dci, J=16.7, 2.5 Hz, 1H), 5.69 (dci, J=10.4, 2.8 Hz, 1H), 5.29-5.24 (m, 1H), 4.63-4.31 (m, 1H), 4.10-3.68 (m, 2H), 3.61 (dd, J=9.3, 5.2 Hz, 1H), 3.53-3.41 (m, 1H), 3.31 (ci, J=4.7 Hz, 3H), 2.96 (t, J=5.2 Hz, 3H), 2.67 (s, 3H), 2.32 (s, 1H).

### Example 275: 1-((3S,5R)-1-Acryloyl-5-(methoxymethyl)pyrrolidin-3-yl)-3-((1-methyl-1H-indazol-4-yl)ethynyl)-5-(methylamino)-1H-pyrazole-4-carboxamide

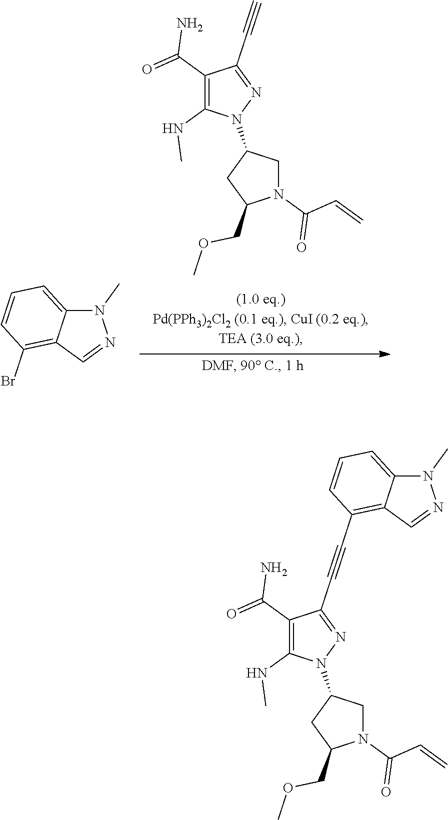 [SMILES: C#Cc1nn([C@H]2C[C@H](COC)N(C(=O)C=C)C2)c(NC)c1C(N)=O ; Cn1ncc2c(Br)cccc21 ; C=CC(=O)N1C[C@@H](n2nc(C#Cc3cccc4c3cnn4C)c(C(N)=O)c2NC)C[C@@H]1COC]

1-[(3S,5R)-5-(methoxymethyl)-1-(prop-2-enoyl)pyrrolidin-3-yl]-5-(methylamino)-3-[2-(1-methylindazol-4-yl)ethynyl]pyrazole-4-carboxamide. MS ESI calculated for C₂₄H₂₇N₇O₃ [M+H]⁺, 462.50, found 462.20; ¹H NMR (400 MHz, CDCl₃) δ 8.14 (d, J=1.0 Hz, 1H), 7.53-7.45 (m, 3H), 7.41-7.38 (m, 2H), 6.75 (dd, J=47.1, 6.1 Hz, 2H), 6.60-6.37 (m, 1H), 5.79-5.72 (m, 1H), 5.58-5.52 (m, 1H), 5.41-5.22 (m, 1H), 4.62-4.03 (m, 5H), 4.03-3.88 (m, 1H), 3.55-3.43 (m, 1H), 3.40 (d, J=4.2 Hz, 3H), 3.06 (dd, J=15.5, 5.9 Hz, 3H), 2.80-2.67 (m, 1H), 2.45-2.29 (m, 1H).

### Example 276: 3-[2-(1-Cyclopropylindazol-4-yl)ethynyl]-1-[(3S,5R)-5-(methoxymethyl)-1-(prop-2-enoyl)pyrrolidin-3-yl]-5-(methylamino)pyrazole-4-carboxamide

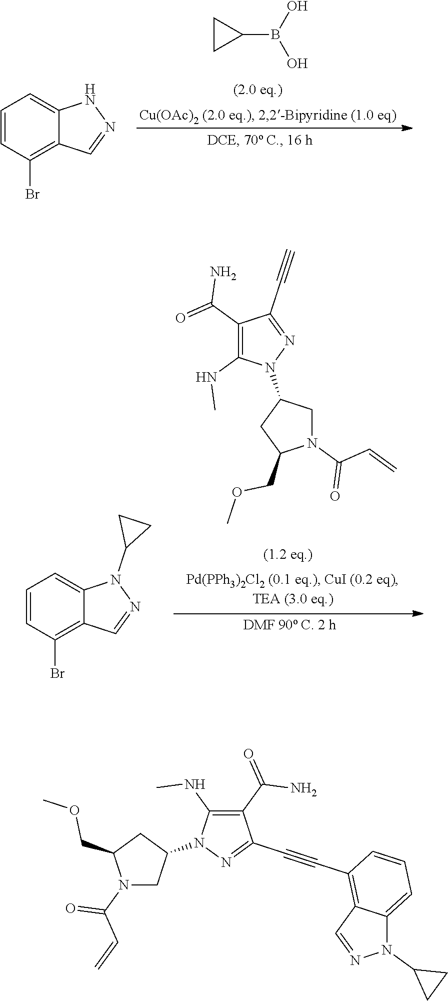 [SMILES: OB(O)C1CC1 ; Brc1cccc2[nH]ncc12 ; Brc1cccc2c1cnn2C1CC1 ; C#Cc1nn([C@H]2C[C@H](COC)N(C(=O)C=C)C2)c(NC)c1C(N)=O ; C=CC(=O)N1C[C@@H](n2nc(C#Cc3cccc4c3cnn4C3CC3)c(C(N)=O)c2NC)C[C@@H]1COC]

### Step 1: 4-Bromo-1-cyclopropylindazole

To a stirred mixture of 4-bromo-1H-indazole (2.00 g, 10.15 mmol), cyclopropylboronic acid (1.74 g, 20.30 mmol), 2,2′-Bipyridine (1.59 g, 10.15 mmol) in DCE (100.00 mL) was added Cu(OAc)₂ (3.69 g, 20.30 mmol) at room temperature under air atmosphere. The reaction mixture was stirred for 16 h at 70° C. under air atmosphere. The resulting mixture was diluted with water (50 mL) and extracted with DCM (3×100 mL). The combined organic layers was washed with brine (2×50 mL), dried over anhydrous Na₂SO₄, filtered and concentrated under reduced pressure. The residue was purified by silica gel column chromatography, eluted with PE/EA (4/1). The fractions contained desired product were combined and concentrated to afford 4-bromo-1-cyclopropylindazole (0.80 g, 33%) as a colorless oil. MS ESI calculated for C₁₀H₉BrN₂ [M+H]⁺, 236.99, 238.99, found 237.00, 239.00; ¹H NMR (400 MHz, Chloroform-d) δ 7.94 (d, J=0.9 Hz, 1H), 7.54-7.50 (m, 1H), 7.34-7.18 (m, 2H), 3.64-3.55 (m, 1H), 1.30-1.12 (m, 4H).

### Step 2: 3-[2-(1-Cyclopropylindazol-4-yl)ethynyl]-1-[(3S,5R)-5-(methoxymethyl)-1-(prop-2-enoyl)pyrrolidin-3-yl]-5-(methylamino)pyrazole-4-carboxamide

To a stirred mixture of 4-bromo-1-cyclopropylindazole (0.10 g, 0.42 mmol) and 3-ethynyl-1-[(3S,5R)-5-(methoxymethyl)-1-(prop-2-enoyl)pyrrolidin-3-yl]-5-(methylamino)pyrazole-4-carboxamide (0.16 g, 0.50 mmol) in DMF (1.00 mL) were added Pd(PPh₃)₂Cl₂ (29.60 mg, 0.04 mmol), CuI (16.06 mg, 0.08 mmol) and TEA (0.12 g, 1.26 mmol). The reaction mixture was degassed with nitrogen for three times and stirred for 2 h at 90° C. The resulting mixture was concentrated under vacuum. The residue was purified by silica gel column chromatography, eluted with DCM/MeOH (20/1) to afford the crude product which was further purified by Prep-HPLC with the following conditions: Column: XSelect CSH Prep C18 OBD Column, 19×250 mm, 5 μm; Mobile Phase A: water (10 mmol/L NH₄HCO₃), Mobile Phase B: DCM:EtOH=9:1-HPLC; Flow rate: 25 mL/min; Gradient: 57% B to 57% B in 20 min, 57% B; Wave Length: 254 nm; RT1: 15.2 min. The fractions contained desired product were combined and concentrated to afford 3-[2-(1-cyclopropylindazol-4-yl)ethynyl]-1-[(3S,5R)-5-(methoxymethyl)-1-(prop-2-enoyl)pyrrolidin-3-yl]-5-(methylamino)pyrazole-4-carboxamide (36.50 mg, 17%) as a white solid. MS ESI calculated for C₂₆H₂₉N₇O₃ [M+H]⁺, 488.23, found 488.25; ¹H NMR (400 MHz, Chloroform-d) δ 8.07 (d, J=1.0 Hz, 1H), 7.71-7.63 (m, 1H), 7.43-7.34 (m, 2H), 6.79 (d, J=6.1 Hz, 1H), 6.57-6.34 (m, 2H), 5.70-5.67 (m, 1H), 5.51-5.46 (m, 1H), 5.34 (s, 1H), 4.56 (d, J=9.1 Hz, 1H), 4.11-4.09 (m, 1H), 4.09-3.96 (m, 1H), 4.00-3.86 (m, 11H), 3.61-3.59 (m, 1H), 3.53-3.40 (m, 1H), 3.37 (d, J=4.2 Hz, 3H), 3.03-2.96 (m, 3H), 2.77-2.65 (m, 1H), 2.35-2.31 (m, 1H), 1.29-1.14 (m, 5H).

### Example 277: 3-(2-(Imidazo[1,5-a]pyridin-8-yl)ethynyl)-1-[(3S,5R)-5-(methoxymethyl)-1-(prop-2-enoyl)pyrrolidin-3-yl]-5-(methylamino)pyrazole-4-carboxamide

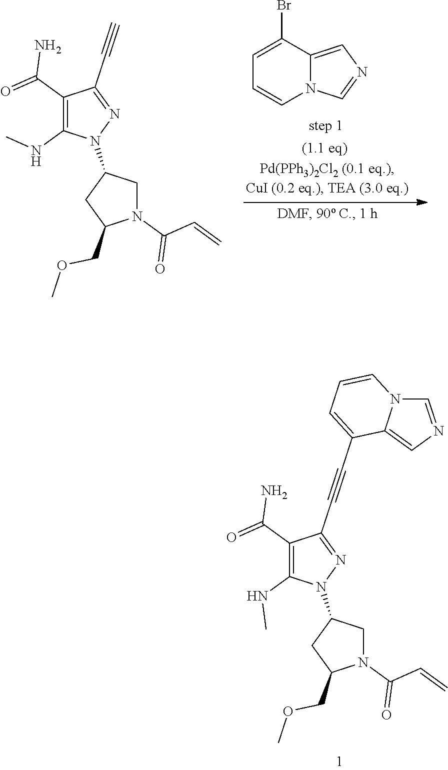 [SMILES: C#Cc1nn([C@H]2C[C@H](COC)N(C(=O)C=C)C2)c(NC)c1C(N)=O ; Brc1cccn2cncc12 ; C=CC(=O)N1C[C@@H](n2nc(C#Cc3cccn4cncc34)c(C(N)=O)c2NC)C[C@@H]1COC]

To a stirred solution of 3-ethynyl-1-[(3S,5R)-5-(methoxymethyl)-1-(prop-2-enoyl)pyrrolidin-3-yl]-5-(methylamino)pyrazole-4-carboxamide (0.15 g, 0.45 mmol) and 8-bromoimidazo[1,5-a]pyridine (98.11 mg, 0.49 mmol) in DMF (1.50 mL) were added Pd(PPh₃)₂Cl₂ (31.77 mg, 0.04 mmol) and CuI (17.24 mg, 0.09 mmol) and TEA (0.14 g, 1.35 mmol). The reaction mixture was degassed with nitrogen for three times and stirred for 1 h at 90° C. The resulting mixture was concentrated under reduced pressure. The residue was purified by silica gel column chromatography, eluted with DCM/MeOH (5/1) to afford the crude product. The crude product (150 mg) was purified by Prep-HPLC with the following conditions: Column: XBridge Shield RP18 OBD Column, 19×250 mm, 10 μm; Mobile Phase A: water (10 mmol/L NH₄HCO₃), Mobile Phase B: MeOH—HPLC; Flow rate: 25 mL/min; Gradient: 50% B to 55% B in 8 min, 55% B; Wave Length: 254 nm; RT1: 6.7 min. The fractions contained desired product were combined and concentrated to afford 3-(2-{imidazo[1,5-a]pyridin-8-yl}ethynyl)-1-[(3S,5R)-5-(methoxymethyl)-1-(prop-2-enoyl)pyrrolidin-3-yl]-5-(methylamino)pyrazole-4-carboxamide (60.90 mg, 30%) as a light yellow solid. MS ESI calculated for C₂₃H₂₅N₇O₃ [M+H]⁺, 448.49, found 448.25; ¹H NMR (400 MHz, DMSO-d₆) S 8.61-8.37 (m, 2H), 7.48-7.45 (s, 2H), 7.14-7.40 (m, 1H), 6.92-6.90 (s, 1H), 6.78-6.55 (m, 2H), 6.51-6.38 (m, 1H), 6.17-6.16 (m, 1H), 5.69-6.68 (m, 1H), 5.23-5.22 (m, 1H), 4.60-4.36 (m, 1H), 4.08-3.70 (m, 2H), 3.65-3.40 (m, 2H), 3.35-3.33 (m, 3H), 2.93-2.92 (m, 3H), 2.69-2.54 (m, 1H), 2.31-2.30 (m, 1H).

### Example 278: 3-(2-{Imidazo[1,5-a]pyridin-5-yl}ethynyl)-1-[(3S,5R)-5-(methoxymethyl)-1-(prop-2-enoyl)pyrrolidin-3-yl]-5-(methylamino)pyrazole-4-carboxamide

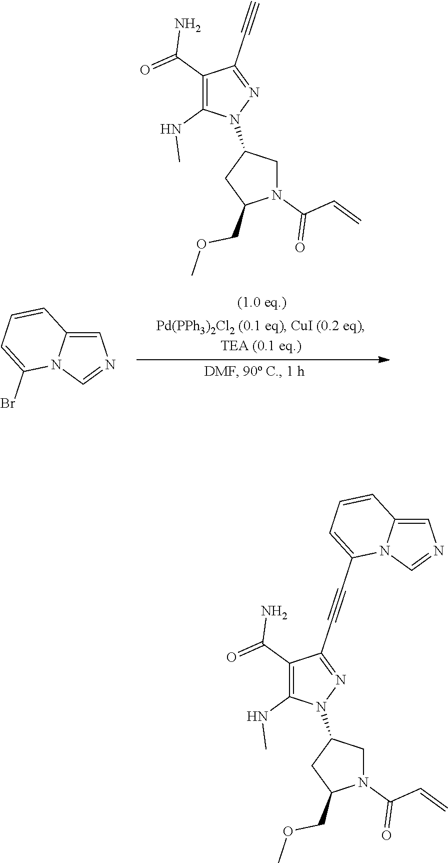 [SMILES: C#Cc1nn([C@H]2C[C@H](COC)N(C(=O)C=C)C2)c(NC)c1C(N)=O ; Brc1cccc2cncn12 ; C=CC(=O)N1C[C@@H](n2nc(C#Cc3cccc4cncn34)c(C(N)=O)c2NC)C[C@@H]1COC]

3-(2-{imidazo[1,5-a]pyridin-5-yl}ethynyl)-1-[(3S,5R)-5-(methoxymethyl)-1-(prop-2-enoyl)pyrrolidin-3-yl]-5-(methylamino)pyrazole-4-carboxamide. MS ESI calculated for C₂₃H₂₅N₇O₃ [M+H]⁺, 448.20, found 448.15; ¹H NMR (400 MHz, CDCl₃) δ 8.75-8.63 (s, 1H), 7.73 (d, J=9.2 Hz, 1H), 7.71 (s, 1H), 7.15 (s, 2H), 7.13-7.12 (m, 1H), 6.87-6.84 (m, 1H), 6.62-6.55 (m, 1H), 6.19-6.13 (m, 2H), 5.70 (d, J=4.0 Hz, 1H), 5.23-5.21 (m, 1H), 4.01 (m, 1H), 3.86-3.81 (m, 2H), 3.57-3.55 (m, 1H), 3.51-3.50 (m, 1H), 3.44 (s, 3H), 2.89 (s, 3H), 2.55-2.53 (m, 1H), 2.33-2.30 (m, 1H).

### Example 279: 3-(2-{6-Chloro-1-[(1R)-2,2-difluorocyclopropyl]-1,3-benzodiazol-5-yl}ethynyl)-1-[(3S,5R)-5-(methoxymethyl)-1-(prop-2-enoyl)pyrrolidin-3-yl]-5-(methylamino)pyrazole-4-carboxamide

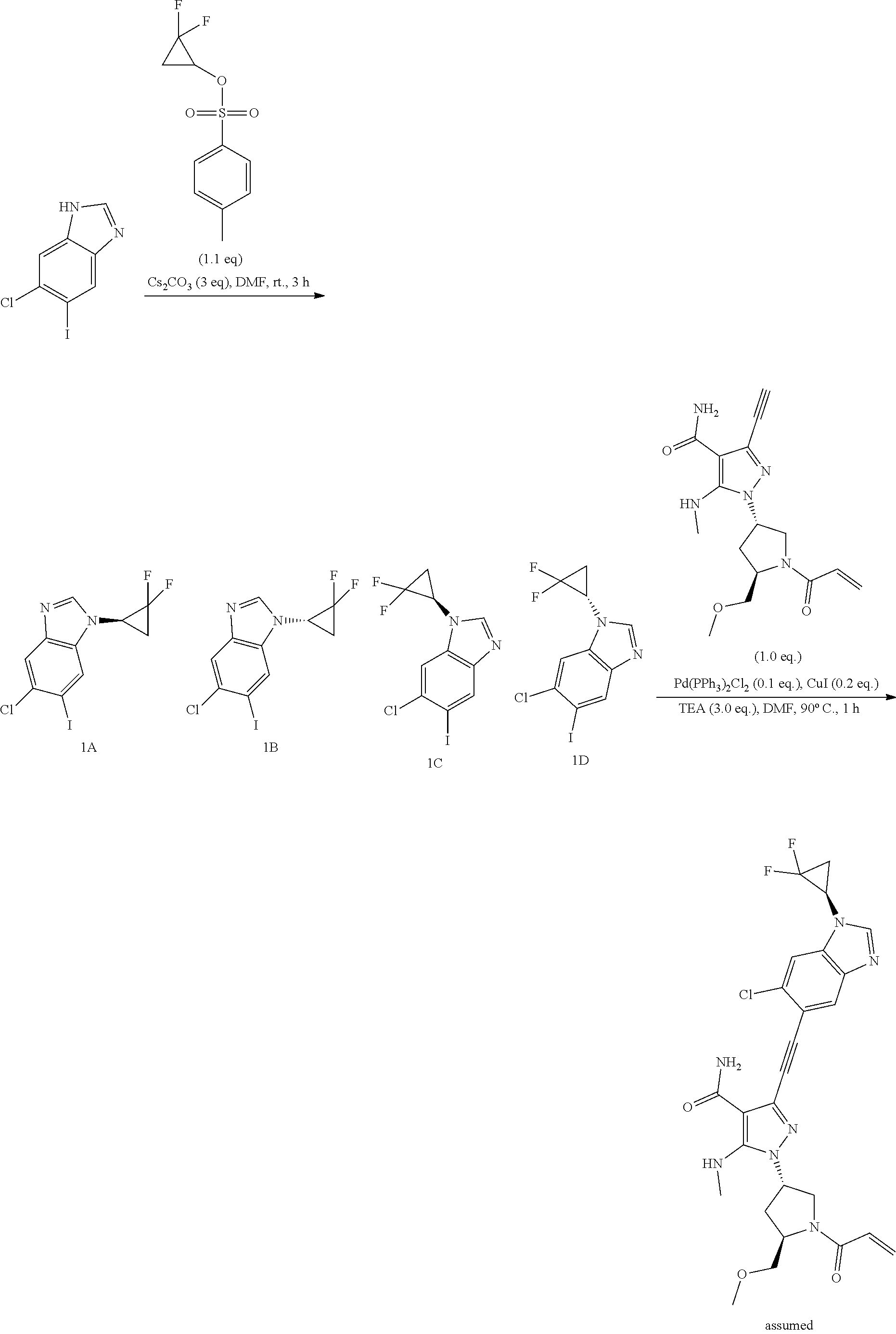

Step 1: (R)-5-chloro-1-(2,2-difluorocyclopropyl)-6-iodo-1H-benzo[d]imidazole & (S)-5-chloro-1-(2,2-difluorocyclopropyl)-6-iodo-1H-benzo[d]imidazole & (R)-6-chloro-1-(2,2-difluorocyclopropyl)-5-iodo-1H-benzo[d]imidazole & (S)-6-chloro-1-(2,2-difluorocyclopropyl)-5-iodo-1H-benzo[d]imidazole

To a stirred mixture of 5-chloro-6-iodo-3H-1,3-benzodiazole (0.45 g, 1.61 mmol) and 2,2-difluorocyclopropyl 4-methylbenzenesulfonate (0.44 g, 1.77 mmol) in DMF (2.00 mL) was added Cs₂CO₃ (1.58 g, 4.84 mmol) in portions at room temperature under nitrogen atmosphere. The reaction mixture was stirred for 3 h at room temperature under nitrogen atmosphere. The resulting mixture was filtered, the filtrate was purified by reverse flash chromatography with the following conditions: column, C18 silica gel; mobile phase, ACN in water (10 mmol/L NH₄HCO₃), 10% to 50% gradient in 30 min; detector: UV 254 nm to afford 6-chloro-1-(2,2-difluorocyclopropyl)-5-iodo-1,3-benzodiazole (320 mg). The crude product was purified by Prep-HPLC with the following conditions: Column: CHIRALPAK IG, 2×25 cm, 5 μm; Mobile Phase A: Hex (0.5% 2 M NH₃-MeOH)—HPLC, Mobile Phase B: MeOH:EtOH=1:1-HPLC; Flow rate: 20 mL/min; Gradient: 15% B to 15% B in 17 min; Wave Length: 220/254 nm; RT1: 11.40 min, RT2: 13.65 min; Sample Solvent: MeOH:DCM=1:1; Injection Volume: 0.3 mL. The faster peak contained desired product were combined and concentrated to afford (R)-5-chloro-1-(2,2-difluorocyclopropyl)-6-iodo-1H-benzo[d]imidazole (30.20 mg, 6%) as an off-white solid, MS ESI calculated for C₁₀H₆ClF₂IN₂ [M+H]⁺, 355.50, found 354.80; ¹H NMR (400 MHz, CDCl₃) δ 7.96 (s, 1H), 7.92 (d, J=1.4 Hz, 2H), 3.90-3.82 (m, 1H), 2.29-2.24 (m, 1H), 2.12-2.03 (m, 1H). And (S)-5-chloro-1-(2,2-difluorocyclopropyl)-6-iodo-1H-benzo[d]imidazole (40.10 mg, 7%) as an off-white solid, MS ESI calculated for C₁₀H₆ClF₂IN₂ [M+H]⁺, 355.50, found 354.80; ¹H NMR (400 MHz, CDCl₃) δ 7.97 (s, 1H), 7.93 (s, 2H), 3.96-3.85 (m, 1H), 2.29-2.23 (m, 1H), 2.02-1.98 (m, 1H).

The slower peak contained desired product were combined and concentrated to afford (R)-6-chloro-1-(2,2-difluorocyclopropyl)-5-iodo-1H-benzo[d]imidazole (39.89 mg, 7%) as as an off-white solid, MS ESI calculated for C₁₀H₆ClF₂IN₂ [M+H]⁺, 355.50, found, 354.80; ¹H NMR (400 MHz, CDCl₃) δ 8.35-8.29 (m, 1H), 7.96-7.72 (m, 1H), 7.65-7.60 (m, 1H), 3.93-3.89 (m, 1H), 2.36-2.22 (m, 1H), 2.02 (ddt, J=14.4, 10.0, 5.3 Hz, 1H). And (S)-6-chloro-1-(2,2-difluorocyclopropyl)-5-iodo-1H-benzo[d]imidazole (39.62 mg, 7%) as an off-white solid. MS ESI calculated for C₁₀H₆ClF₂IN₂ [M+H]⁺, 355.50, found, 354.80; ¹H NMR (400 MHz, CDCl₃) δ 8.29 (s, 1H), 7.91 (d, J=1.7 Hz, 1H), 7.60 (s, 1H), 3.94-3.90 (m, 1H), 2.29-2.26 (m, 1H), 2.08-1.95 (m, 1H).

### Step 2: 3-(2-{6-Chloro-1-[(1R)-2,2-difluorocyclopropyl]-1,3-benzodiazol-5-yl}ethynyl)-1-[(3S,5R)-5-(methoxymethyl)-1-(prop-2-enoyl)pyrrolidin-3-yl]-5-(methylamino)pyrazole-4-carboxamide

To a stirred solution of (R)-6-chloro-1-(2,2-difluorocyclopropyl)-5-iodo-1H-benzo[d]imidazole (40 mg, 0.11 mmol) and 3-ethynyl-1-[(3S,5R)-5-(methoxymethyl)-1-(prop-2-enoyl)pyrrolidin-3-yl]-5-(methylamino)pyrazole-4-carboxamide (37.39 mg, 0.11 mmol) in DMF (0.40 mL) were added Pd(PPh₃)₂Cl₂ (7.92 mg, 0.01 mmol) and TEA (34.25 mg, 0.34 mmol). The reaction mixture was degassed with argon for three times and stirred for 1 h at 90° C. The resulting mixture was concentrated under reduced pressure. The residue was purified by silica gel column chromatography, eluted with CH₂Cl₂/MeOH (10/1) to afford the crude product which was further purified by Prep-HPLC with the following conditions: Column: XBridge Prep OBD C18 Column, 30×150 mm, 5 μm; Mobile Phase A: water (10 mmol/L NH₄HCO₃), Mobile Phase B: ACN; Flow rate: 60 mL/min; Gradient: 28% B to 38% B in 8 min, 38% B; Wave Length: 254 nm; RT1: 7 min. The fractions contained desired product were combined and concentrated to afford 3-(2-{6-chloro-1-[(1R)-2,2-difluorocyclopropyl]-1,3-benzodiazol-5-yl}ethynyl)-1-[(3S,5R)-5-(methoxymethyl)-1-(prop-2-enoyl)pyrrolidin-3-yl]-5-(methylamino)pyrazole-4-carboxamide (stereochemistry arbitrarily assigned) (20.40 mg, 32%) as an off-white solid. MS ESI calculated for C₂₆H₂₆ClF₂N₇O₃ [M+H]⁺, 558.18, found, 558.20; ¹H NMR (400 MHz, CDCl₃) δ 8.12 (d, J=4.9 Hz, 2H), 7.59 (s, 1H), 6.60-6.36 (m, 2H), 5.73 (dd, J=8.7, 3.6 Hz, 1H), 5.59-5.46 (m, 1H), 5.43-5.28 (m, 2H), 4.58 (d, J=8.9 Hz, 1H), 4.17-3.88 (m, 4H), 3.55-3.43 (m, 1H), 3.39 (d, J=4.8 Hz, 3H), 3.05 (d, J=15.2 Hz, 3H), 2.79-2.67 (m, 1H), 2.39-2.34 (m, 2H), 2.08-2.03 (m, 1H).

### Example 280: 3-(2-{6-Chloro-1-[(1S)-2,2-difluorocyclopropyl]-1,3-benzodiazol-5-yl}ethynyl)-1-[(3S,5R)-5-(methoxymethyl)-1-(prop-2-enoyl)pyrrolidin-3-yl]-5-(methylamino)pyrazole-4-carboxamide

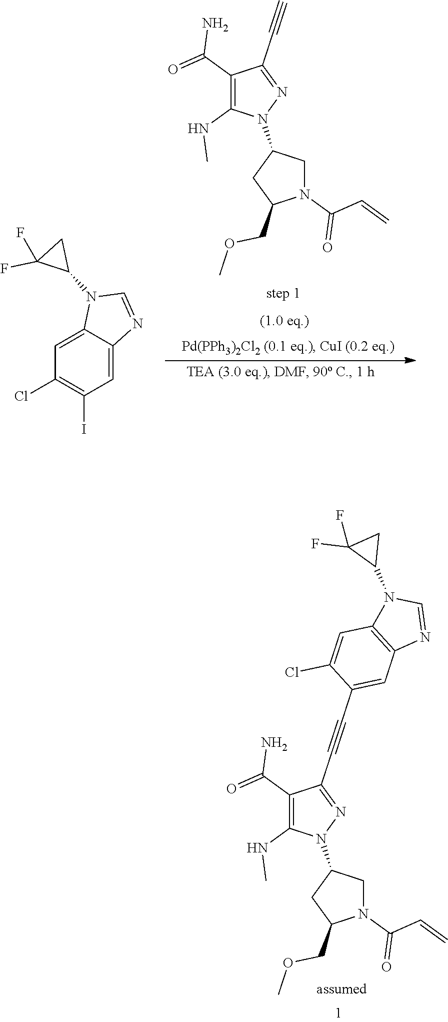

To a stirred solution of 6-chloro-1-[(1S)-2,2-difluorocyclopropyl]-5-iodo-1,3-benzodiazole (48.00 mg, 0.13 mmol), 3-ethynyl-1-[(3S,5R)-5-(methoxymethyl)-1-(prop-2-enoyl)pyrrolidin-3-yl]-5-(methylamino)pyrazole-4-carboxamide (44.87 mg, 0.13 mmol), Pd(PPh₃)₂Cl₂ (9.50 mg, 0.01 mmol) and CuI (5.16 mg, 0.03 mmol) in DMF (1.00 mL) was added TEA (41.10 mg, 0.41 mmol). The reaction mixture was degassed with nitrogen for three times and stirred for 1 h at 90° C. The resulting mixture was concentrated under reduced pressure. The residue was purified by silica gel column chromatography, eluted with DCM/MeOH (10/1) to afford the crude product which was further purified by reverse phase flash with the following conditions: Column: XBridge Prep OBD C18 Column, 30×150 mm, 5 μm; Mobile Phase A: water (10 mmol/L NH₄HCO₃), Mobile Phase B: ACN; Flow rate: 60 mL/min; Gradient: 30% B to 35% B in 8 min, Wave Length: 254 nm. The fractions contained desired product were combined and concentrated to afford 3-(2-{6-chloro-1-[(1S)-2,2-difluorocyclopropyl]-1,3-benzodiazol-5-yl}ethynyl)-1-[(3S,5R)-5-(methoxymethyl)-1-(prop-2-enoyl)pyrrolidin-3-yl]-5-(methylamino)pyrazole-4-carboxamide (stereochemistry arbitrarily assigned) (10.70 mg, 14%) as a white solid. MS ESI calculated for C₂₆H₂₆ClF₂N₇O₃ [M+H]+, 558.18, found 558.25; ¹H NMR (400 MHz, CDCl₃) δ 8.13 (s, 1H), 8.03 (s, 1H), 7.59 (s, 1H), 7.17 (s, 1H), 6.83 (d, J=6.7 Hz, H), 6.44 (d, J=9.0 Hz, 2H), 5.73-5.71 (m, 1H), 5.57-5.48 (m, 1H), 5.36 (s, 1H), 4.58 (d, J=9.0 Hz, 1H), 4.15-4.04 (m, 2H), 3.93 (s, 2H), 3.60-3.31 (m, 4H), 3.07-3.02 (m, 3H), 2.77-2.72 (m, 1H), 2.34 (s, 2H), 2.04 (t, J=6.8 Hz, 1H).

### Example 281: 3-[2-(6-chloro-4-methylquinolin-7-yl)ethynyl]-1-[(3S,5R)-5-(methoxymethyl)-1-(prop-2-enoyl)pyrrolidin-3-yl]-5-(methylamino)pyrazole-4-carboxamide

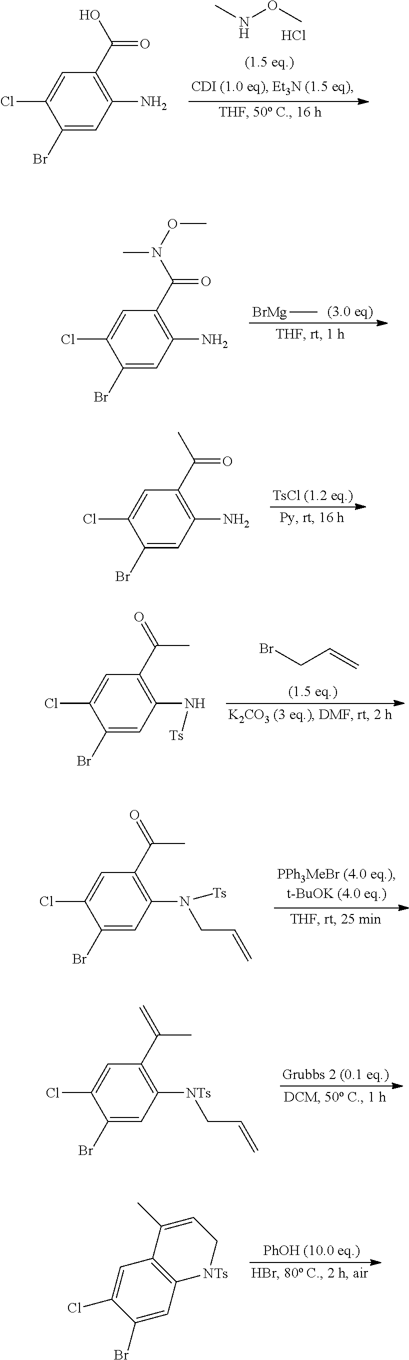

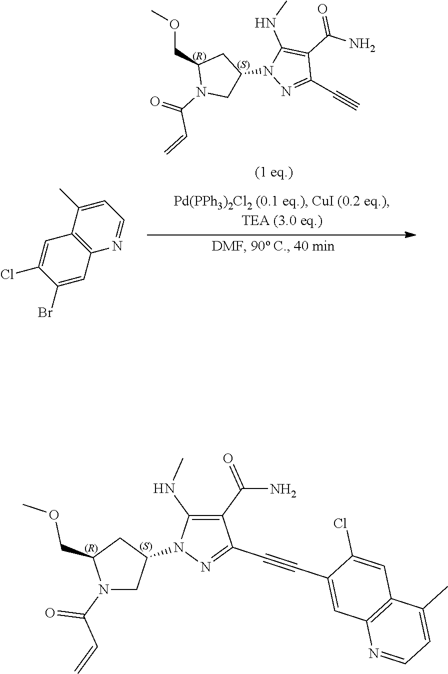

3-[2-(6-chloro-4-methylquinolin-7-yl)ethynyl]-1-[(3S,5R)-5-(methoxymethyl)-1-(prop-2-enoyl)pyrrolidin-3-yl]-5-(methylamino)pyrazole-4-carboxamide. MS ESI calculated for C₂₆H₂₇ClN₆O₃[M+H]⁺, 507.18, found 507.25; ¹H NMR (400 MHz, CDCl₃) δ 8.84 (s, 1H), 8.44 (s, 1H), 8.09 (s, 1H), 7.32 (s, 1H), 7.12 (s, 1H), 6.85 (s, 1H), 6.48-6.40 (m, 2H), 5.85-5.73 (m, 1H), 5.57-5.32 (m, 2H), 4.59 (d, J=9.0 Hz, 1H), 4.22-4.10 (m, 2H), 3.93 (d, J=8.5 Hz, 1H), 3.47 (d, J=9.6 Hz, 1H), 3.40 (d, J=4.6 Hz, 3H), 3.20-3.06 (m, 3H), 2.71 (s, 4H), 2.50-2.34 (m, 1H).

### Example 282: 1-((3S,5R)-1-Acryloyl-5-(methoxymethyl)pyrrolidin-3-yl)-3-((6-fluoro-3,4-dimethylquinolin-7-yl)ethynyl)-5-(methylamino)-1H-pyrazole-4-carboxamide

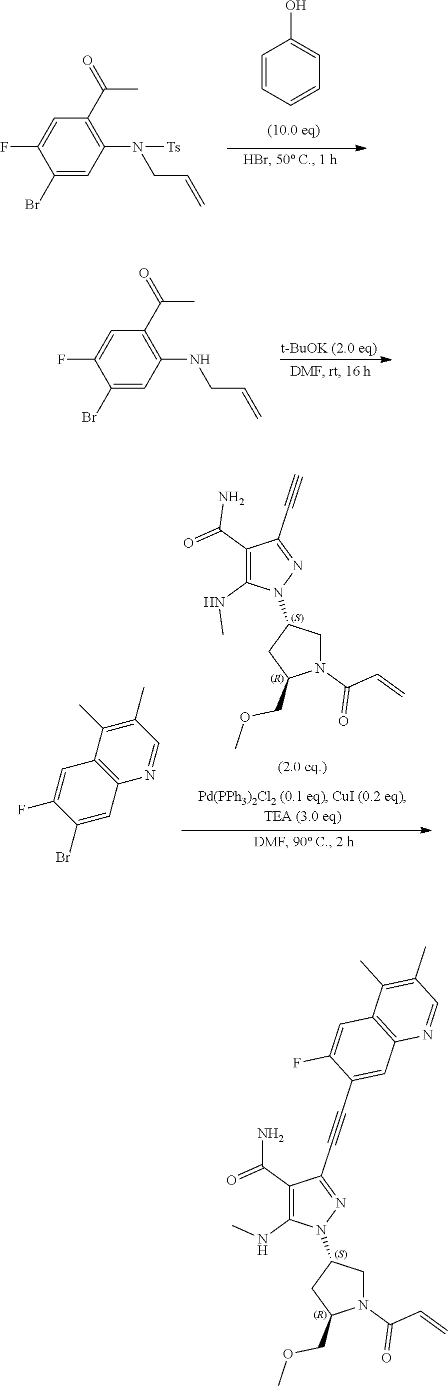

### Step 1: 1-[4-Bromo-5-fluoro-2-(prop-2-en-1-ylamino)phenyl]ethanone

To a stirred mixture of N-(2-acetyl-5-bromo-4-fluorophenyl)-N-allyl-4-methylbenzenesulfonamide (0.50 g, 1.17 mmol) in HBr (5.00 mL) was added phenol (1.10 g, 11.73 mmol) at room temperature under nitrogen atmosphere. The reaction mixture was stirred for 1 h at 50° C. under nitrogen atmosphere. The resulting mixture was concentrated under vacuum. The residue was purified by silica gel column chromatography, eluted with PE/EA (5/1). The fractions contained desired product were combined and concentrated to afford 1-[4-bromo-5-fluoro-2-(prop-2-en-1-ylamino)phenyl]ethanone (0.24 g, 75%) as a yellow solid. MS ESI calculated for C₁₁H₁₁BrFNO [M+H]⁺, 272.00, 274.00, found 271.90, 273.90; ¹H NMR (400 MHz, Chloroform-d) δ 8.85 (s, 1H), 7.49 (d, J=9.6 Hz, 1H), 6.88 (d, J=5.7 Hz, 1H), 5.93 (m, 1H), 5.35-5.15 (m, 2H), 3.86 (m, 2H), 2.57 (s, 3H).

### Step 2: 7-bromo-6-fluoro-3,4-dimethylquinoline

To a stirred mixture of 1-[4-bromo-5-fluoro-2-(prop-2-en-1-ylamino)phenyl]ethanone (0.20 g, 0.74 mmol) in DMF (7.00 mL) was added t-BuOK (0.17 g, 1.47 mmol) in portions at room temperature under nitrogen atmosphere. The reaction mixture was stirred for 16 h at room temperature under nitrogen atmosphere. The residue was purified by reverse flash chromatography with the following conditions: column: C18 silica gel; mobile phase: ACN in water (10 mmol/L NH₄HCO₃), 30% to 70% gradient in 25 min; detector: UV 254 nm. The fractions contained desired product were combined and concentrated to afford 7-bromo-6-fluoro-3,4-dimethylquinoline (63.00 mg, 34%) as a yellow solid. MS ESI calculated for C₁₁H₉BrFN [M+H]⁺, 253.99, 255.99. found 253.65, 255.65.

### Step 3. 1-((3S,5R)-1-Acryloyl-5-(methoxymethyl)pyrrolidin-3-yl)-3-((6-fluoro-3,4-dimethylquinolin-7-yl)ethynyl)-5-(methylamino)-1H-pyrazole-4-carboxamide

To a stirred mixture of 7-bromo-6-fluoro-3,4-dimethylquinoline (50.00 mg, 0.20 mmol) and 1-((3S,5R)-1-acryloyl-5-(methoxymethyl)pyrrolidin-3-yl)-3-ethynyl-5-(methylamino)-1H-pyrazole-4-carboxamide (0.13 g, 0.40 mmol) in DMF (0.50 mL) was added Pd(PPh₃)₂Cl₂ (13.81 mg, 0.02 mmol), CuI (7.50 mg, 0.04 mmol) TEA (59.73 mg, 0.59 mmol) at room temperature under nitrogen atmosphere. The reaction mixture was degassed with nitrogen for three times and stirred for 2 h at 90° C. The resulting mixture was concentrated under vacuum. The residue was purified by silica gel column chromatography, eluted with DCM/MeOH (10/1) to afford the crude product which was further purified by Prep-HPLC with the following conditions: Column: XBridge Shield RP18 OBD Column, 30×150 mm, 5 μm; Mobile Phase A: water (10 mmol/L NH₄HCO₃), Mobile Phase B: ACN; Flow rate: 60 mL/min; Gradient: 34% B to 44% B in 10 min, 44% B; Wave Length: 220 nm; RT1: 9 min. The fractions contained desired product were combined and concentrated to afford 1-((3S,5R)-1-acryloyl-5-(methoxymethyl)pyrrolidin-3-yl)-3-((6-fluoro-3,4-dimethylquinolin-7-yl)ethynyl)-5-(methylamino)-1H-pyrazole-4-carboxamide (25.50 mg, 25%) as an off-white solid. MS ESI calculated for C₂₇H₂₉FN₆O₃[M+H]⁺, 505.23, found 505.35; ¹H NMR (400 MHz, Chloroform-d) δ 8.71 (s, 1H), 8.30 (d, J=7.1 Hz, 1H), 7.67 (d, J=11.1 Hz, 1H), 7.07 (s, 1H), 6.86 (d, J=6.0 Hz, 1H), 6.60-6.37 (m, 2H), 5.73 (m, 1H), 5.62-5.20 (m, 2H), 4.58 (d, J=9.0 Hz, 1H), 4.18-3.88 (m, 3H), 3.56-3.43 (m, 1H), 3.39 (d, J=4.7 Hz, 3H), 3.06 (dd, J=15.7, 5.9 Hz, 3H), 2.80-2.67 (m, 1H), 2.58 (s, 3H), 2.50 (s, 3H), 2.37 (m, 1H).

### Example 283: 3-(2-{3-Cyclopropyl-6-fluoroimidazo[1,2-a]pyridin-7-yl}ethynyl)-1-[(3S,5R)-5-(methoxymethyl)-1-(prop-2-enoyl)pyrrolidin-3-yl]-5-(methylamino)pyrazole-4-carboxamide

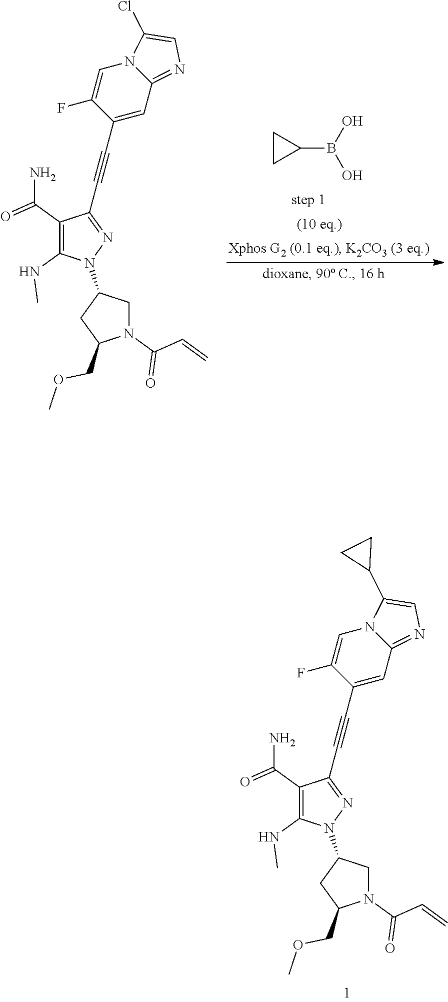 [SMILES: C=CC(=O)N1C[C@@H](n2nc(C#Cc3cc4ncc(Cl)n4cc3F)c(C(N)=O)c2NC)C[C@@H]1COC ; OB(O)C1CC1 ; C=CC(=O)N1C[C@@H](n2nc(C#Cc3cc4ncc(C5CC5)n4cc3F)c(C(N)=O)c2NC)C[C@@H]1COC]

3-(2-{3-cyclopropyl-6-fluoroimidazo[1,2-a]pyridin-7-yl}ethynyl)-1-[(3S,5R)-5-(methoxymethyl)-1-(prop-2-enoyl)pyrrolidin-3-yl]-5-(methylamino)pyrazole-4-carboxamide. MS ESI calculated for C₂₆H₂₈FN₇O₃[M+H]⁺, 506.22, found 506.25; ¹H NMR (400 MHz, CDCl₃) δ 6.88-6.70 (m, 2H), 6.44 (d, J=8.6 Hz, 2H), 5.85-5.73 (m, 1H), 5.52 (d, J=9.6 Hz, 1H), 5.33 (s, 1H), 4.60-4.43 (m, 1H), 4.20-4.06 (m, 2H), 3.93 (d, J=9.7 Hz, 1H), 3.50 (d, J=22.2 Hz, 1H), 3.39 (s, 3H), 3.05 (d, J=15.1 Hz, 3H), 2.75-2.65 (m, 1H), 2.45-2.33 (m, 1H), 1.96 (s, 1H), 1.14 (d, J=7.5 Hz, 2H), 0.77 (s, 2H).

### Example 284: 3-(2-{3-Cyclopropyl-4-methoxypyrazolo[1,5-a]pyridin-6-yl}ethynyl)-1-[(3S,5R)-5-(methoxymethyl)-1-(prop-2-enoyl)pyrrolidin-3-yl]-5-(methylamino)pyrazole-4-carboxamide

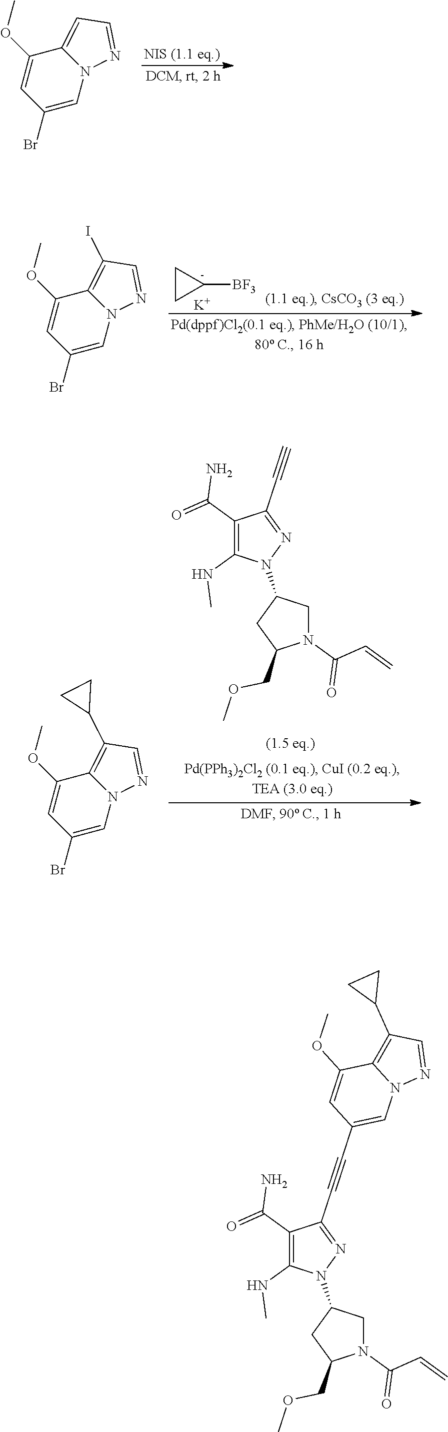

3-(2-{3-cyclopropyl-4-methoxypyrazolo[1,5-a]pyridin-6-yl}ethynyl)-1-[(3S,5R)-5-(methoxymethyl)-1-(prop-2-enoyl)pyrrolidin-3-yl]-5-(methylamino)pyrazole-4-carboxamide. MS ESI calculated for C₂₇H₃₁N₇O₄ [M+H]⁺, 518.24, found 518.40; ¹H NMR (400 MHz, Chloroform-d) δ 8.30 (s, 1H), 7.55 (s, 1H), 6.75 (s, 1H), 6.57-6.40 (m, 2H), 6.35 (s, 1H), 5.72-5.64 (m, 1H), 5.55-5.28 (m, 2H), 4.58 (d, J=8.9 Hz, 1H), 4.16-3.90 (m, 6H), 3.50-3.41 (m, 1H), 3.39 (d, J=4.5 Hz, 3H), 3.06 (d, J=14.2 Hz, 3H), 2.71-2.48 (m, 1H), 2.44-2.30 (m, 2H), 1.01-0.95 (m, 2H), 0.73-0.63 (m, 2H).

### Example 285: 1-((3S,5R)-1-Acryloyl-5-(methoxymethyl)pyrrolidin-3-yl)-3-((7-chloro-2,3-dihydro-1H-benzo[d]pyrrolo[1,2-a]imidazol-6-yl)ethynyl)-5-(methylamino)-1H-pyrazole-4-carboxamide

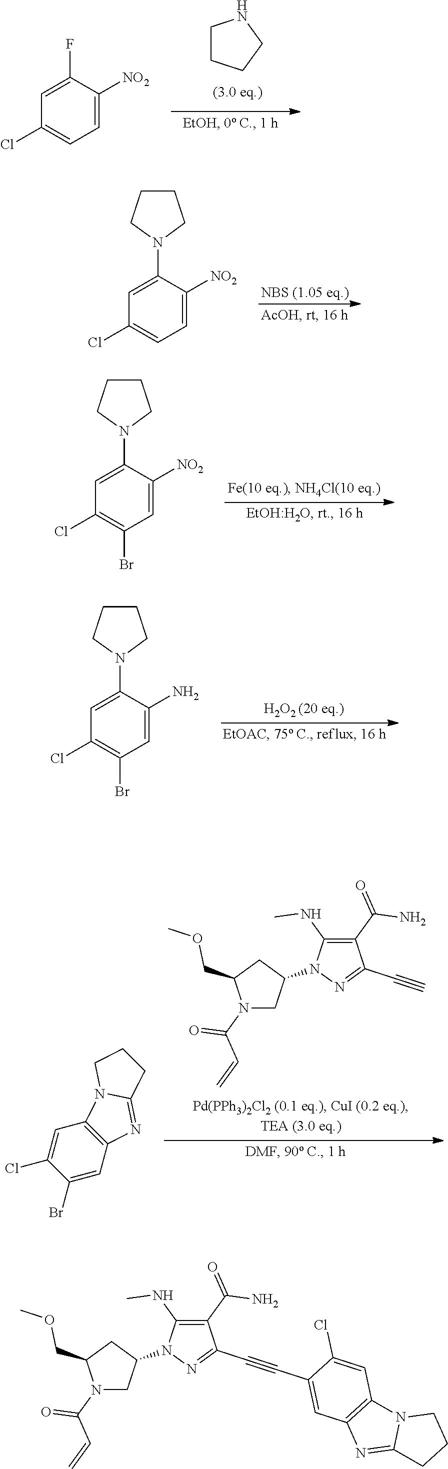

### Step 1: 1-(5-Chloro-2-nitrophenyl) pyrrolidine

To a stirred solution of 4-chloro-2-fluoro-1-nitrobenzene (3.30 g, 18.80 mmol) in EtOH (30 mL) was added pyrrolidine (4.01 g, 56.40 mmol) at 0° C. The reaction mixture was stirred for 1 h at 0° C. The resulting mixture was diluted with water (100 mL) and extracted with EtOAc (3×200 mL). The combined organic layers was washed with brine (3×100 mL), dried over anhydrous Na₂SO₄. After filtration, the filtrate was concentrated under reduced pressure to afford 1-(5-chloro-2-nitrophenyl) pyrrolidine (3.60 g, crude) as an orange solid. MS ESI calculated for C₁₀H₁₁ClN₂O₂[M+H]+, 227.05, found 226.95; ¹H NMR (400 MHz, CDCl₃) δ 7.70 (d, J=8.8 Hz, 1H), 6.91 (d, J=2.1 Hz, 1H), 6.69 (dd, J=8.8, 2.1 Hz, 1H), 3.30-3.19 (m, 4H), 2.09-1.95 (in, 4H).

### Step 2: 1-(4-Bromo-5-chloro-2-nitrophenyl) pyrrolidine

To a stirred solution of 1-(5-chloro-2-nitrophenyl) pyrrolidine (1.80 g, 7.94 mmol) in AcOH (20.00 mL) was added NBS (1.48 g, 8.34 mmol) in portions at 0° C. The reaction mixture was stirred for 16 h at room temperature. The resulting mixture was concentrated under reduced pressure. The residue was purified by silica gel column chromatography, eluted with PE/EA (12/1). The fractions contained desired product were combined and concentrated to afford 1-(4-bromo-5-chloro-2-nitrophenyl) pyrrolidine (2.50 g, 97%) as an orange solid. MS ESI calculated for C₁₀H₁₀BrClN₂O₂ [M+H]⁺, 304.97, 306.97, found 305.05, 307.05.

### Step 3: 5-Bromo-4-chloro-2-(pyrrolidin-1-yl) aniline

To a stirred solution of 1-(4-bromo-5-chloro-2-nitrophenyl) pyrrolidine (2.50 g, 8.18 mmol) and NH₄Cl (4.38 g, 81.82 mmol) in EtOH (20.00 mL) and H₂O (5.00 mL) was added Fe (4.57 g, 81.82 mmol) in portions at room temperature. The reaction mixture was stirred for 16 h at room temperature. The resulting mixture was filtered, the filter cake was washed with EtOAc (5×50 mL). The filtrate was concentrated under reduced pressure. The residue was purified by silica gel column chromatography, eluted with PE/EA (12/1). The fractions contained desired product were combined and concentrated to afford 5-bromo-4-chloro-2-(pyrrolidin-1-yl) aniline (1.80 g, 79%) as a dark red solid. MS ESI calculated for C₁₀H₁₂BrClN₂ [M+H]⁺, 274.99, 276.99, found 275.10, 277.10.

### Step 4: 6-Bromo-7-chloro-2,3-dihydro-1H-benzo[d]pyrrolo[1,2-a]imidazole

To a stirred solution of 5-bromo-4-chloro-2-(pyrrolidin-1-yl)aniline (1.80 g 6.53 mmol) in EtOAc (20.00 mL) was added H₂O₂ (14.81 g, 0.13 mol, 30%) dropwise at room temperature. The reaction mixture was stirred for 16 h at 75° C. The resulting mixture was diluted with water (10 mL) and extracted with EtOAc (5×50 mL). The combined organic layers was washed with brine (2×100 mL), dried over anhydrous Na₂SO₄. After filtration, the filtrate was concentrated under reduced pressure. The residue was purified by silica gel column chromatography, eluted with CH₂Cl₂/MeOH (12/1) The fractions contained desired product were combined and concentrated to afford 6-bromo-7-chloro-2,3-dihydro-1H-benzo[d]pyrrolo[1,2-a]imidazole (1.20 g, 60%) as a brown solid. MS ESI calculated for C₁₀H₈BrClN₂ [M+H]⁺, 270.96, 272.96, found 270.90, 272.90; ¹H NMR (400 MHz, CDCl₃) δ 7.95 (s, 1H), 7.44 (s, 1H), 4.18-4.07 (m, 2H), 3.09 (dd, J=8.3, 7.1 Hz, 2H), 2.83-2.71 (m, 2H).

### Step 5: 1-((3S,5R)-1-Acryloyl-5-(methoxymethyl)pyrrolidin-3-yl)-3-((7-chloro-2,3-dihydro-1H-benzo[d]pyrrolo[1,2-a]imidazol-6-yl)ethynyl)-5-(methylamino)-1H-pyrazole-4-carboxamide

To a stirred mixture of 6-bromo-7-chloro-2,3-dihydro-1H-benzo[d]pyrrolo[1,2-a]imidazole (0.16 g, 0.60 mmol), 3-ethynyl-1-[(3S,5R)-5-(methoxymethyl)-1-(prop-2-enoyl)pyrrolidin-3-yl]-5-(methylamino)pyrazole-4-carboxamide (0.20 g, 0.60 mmol), Pd(PPh₃)₂Cl₂ (42.36 mg, 0.06 mmol) and CuI (22.99 mg, 0.12 mmol) in DMF (2.00 mL) was added TEA (0.18 g, 1.81 mmol) dropwise at room temperature. The reaction mixture was degassed with nitrogen for three times and stirred for 1 h at 90° C. The resulting mixture was concentrated under reduced pressure. The residue was purified by silica gel column chromatography, eluted with CH₂Cl₂/MeOH (10/1) to afford the crude product which was further purified by reverse flash chromatography with the following conditions: column: C18 silica gel; mobile phase: ACN in water (10 mmol/L NH₄HCO₃), 5% to 70% gradient in 40 min; detector: UV 254 nm. The fractions contained desired product were combined and concentrated to afford 1-((3S,5R)-1-acryloyl-5-(methoxymethyl)pyrrolidin-3-yl)-3-((7-chloro-2,3-dihydro-1H-benzo[d]pyrrolo[1,2-a]imidazol-6-yl)ethynyl)-5-(methylamino)-1H-pyrazole-4-carboxamide (26.20 mg, 8%) as an off-white solid. MS ESI calculated for C₂₆H₂₈ClN₇O₃[M+H]⁺, 522.20, found 522.35; ¹H NMR (400 MHz, CDCl₃) δ 8.03 (s, 1H), 7.48 (s, 1H), 7.19 (s, 1H), 6.83 (s, 1H), 6.56-6.40 (m, 2H), 5.76-5.69 (m, 1H), 5.56-5.47 (m, 1H), 5.37 (s, 1H), 4.61-4.54 (m, 1H), 4.21-4.10 (m, 3H), 4.04 (t, J=9.1 Hz, 1H), 3.92 (d, J=9.6 Hz, 1H), 3.56-3.43 (m, 1H), 3.39 (d, J=4.4 Hz, 3H), 3.15 (s, 2H), 3.05 (d, J=15.3 Hz, 3H), 2.83-2.62 (m, 3H), 2.33 (s, 1H).

### Example 287: 1-((3S,5R)-1-Acryloyl-5-(methoxymethyl)pyrrolidin-3-yl)-3-((1-cyclopropyl-5-fluoro-1H-indazol-4-yl)ethynyl)-5-(methylamino)-1H-pyrazole-4-carboxamide

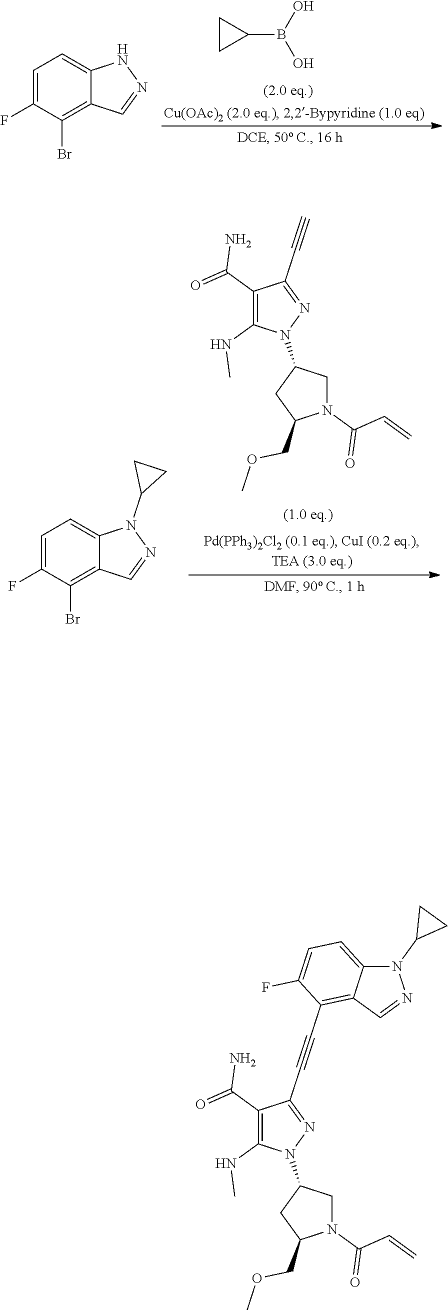 [SMILES: Fc1ccc2[nH]ncc2c1Br ; OB(O)C1CC1 ; Fc1ccc2c(cnn2C2CC2)c1Br ; C#Cc1nn([C@H]2C[C@H](COC)N(C(=O)C=C)C2)c(NC)c1C(N)=O ; C=CC(=O)N1C[C@@H](n2nc(C#Cc3c(F)ccc4c3cnn4C3CC3)c(C(N)=O)c2NC)C[C@@H]1COC]

### Step 1: 4-Bromo-1-cyclopropyl-5-fluoro-1H-indazole

To a stirred mixture of 4-bromo-5-fluoro-1H-indazole (0.50 g, 2.33 mmol), cyclopropylboronic acid (0.40 g, 4.65 mmol) and 2-(pyridin-2-yl)pyridine (0.36 g, 2.33 mmol) in DCE (10.00 mL) was added Cu(OAc)₂ (0.84 g, 4.65 mmol). The reaction mixture was stirred for 16 h at 50° C. under oxygen atmosphere. The resulting mixture was concentrated under reduced pressure. The residue was purified by silica gel column chromatography, eluted with PE/EA (1/1). The fractions contained desired product were combined and concentrated to afford 4-bromo-1-cyclopropyl-5-fluoro-1H-indazole (0.16 g, 27%) as a light orange solid. MS ESI calculated for C₁₀H₈BrFN₂ [M+H]⁺, 254.99, 256.99, found 254.95, 256.95; ¹H NMR (400 MHz, Chloroform-d) δ 7.95 (s, 1H), 7.51 (dd, J=9.0, 3.7 Hz, 1H), 7.21 (t, J=8.8 Hz, 1H), 3.66-3.56 (m, 1H), 1.25-1.19 (m, 4H).

### Step 2: 1-((3S,5R)-1-Acryloyl-5-(methoxymethyl)pyrrolidin-3-yl)-3-((1-cyclopropyl-5-fluoro-1H-indazol-4-yl)ethynyl)-5-(methylamino)-1H-pyrazole-4-carboxamide

To a stirred mixture of 4-bromo-1-cyclopropyl-5-fluoro-1H-indazole (0.16 g, 0.63 mmol), 3-ethynyl-1-[(3S,5R)-5-(methoxymethyl)-1-(prop-2-enoyl)pyrrolidin-3-yl]-5-(methylamino)pyrazole-4-carboxamide (0.21 g, 0.63 mmol), Pd(PPh₃)₂Cl₂ (44.03 mg, 0.06 mmol) and CuI (23.89 mg, 0.13 mmol) in DMF (2.00 mL) was added TEA (0.19 g, 1.88 mmol) at room temperature. The reaction mixture was degassed with nitrogen for three times and stirred for 1 h at 90° C. The resulting mixture was concentrated under reduced pressure. The residue was purified by silica gel column chromatography, eluted with CH₂Cl₂/MeOH (10/1) to afford the crude product. The crude product was purified by reverse flash chromatography with the following conditions: column: C18 silica gel; mobile phase: ACN in water (10 mmol/L NH₄HCO₃), 10% to 50% gradient in 30 min; detector: UV 254 nm. The fractions contained desired product were combined and concentrated to afford 1-((3S,5R)-1-acryloyl-5-(methoxymethyl)pyrrolidin-3-yl)-3-((1-cyclopropyl-5-fluoro-1H-indazol-4-yl)ethynyl)-5-(methylamino)-1H-pyrazole-4-carboxamide (47.40 mg, 15%) as a white solid. MS ESI calculated for C₂₆H₂₈FN₇O₃[M+H]⁺, 506.22, found 506.30; ¹H NMR (400 MHz, DMSO-d₆) δ 8.09 (d, J=3.4 Hz, 1H), 7.87 (dd, J=9.3, 4.0 Hz, 1H), 7.68-7.39 (m, 2H), 6.91 (s, 1H), 6.80-6.43 (m, 2H), 6.17 (dd, J=16.7, 2.4 Hz, 1H), 5.69 (dd, J=10.3, 2.4 Hz, 1H), 5.36-5.14 (m, 1H), 4.47 (d, J=57.4 Hz, 1H), 4.09-3.98 (m, 1H), 3.95-3.69 (m, 2H), 3.68-3.42 (m, 2H), 3.31 (d, J=4.8 Hz, 3H), 2.94 (t, J=5.3 Hz, 3H), 2.72-2.55 (m, 1H), 2.38-2.23 (m, 1H), 1.23-1.04 (m, 4H).

### Example 288: 3-(2-{6-Chloro-3-[(1R)-2,2-difluorocyclopropyl]-1,3-benzodiazol-5-yl}ethynyl)-1-[(3S,5R)-5-(methoxymethyl)-1-(prop-2-enoyl)pyrrolidin-3-yl]-5-(methylamino)pyrazole-4-carboxamide

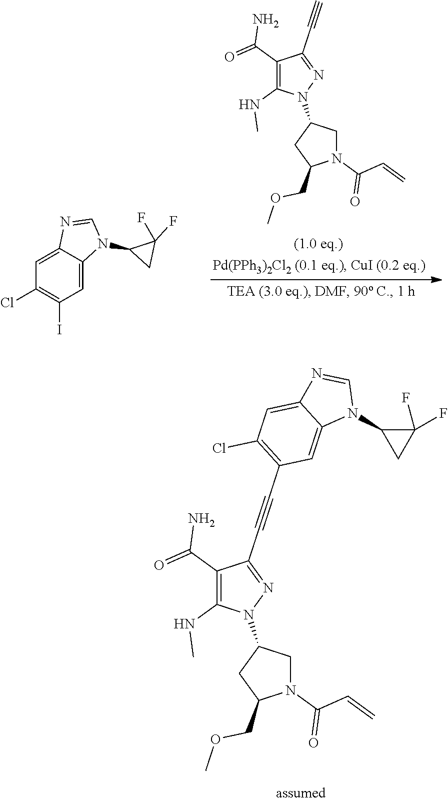 [SMILES: FC1(F)C[C@H]1n1cnc2cc(Cl)c(I)cc21 ; C#Cc1nn([C@H]2C[C@H](COC)N(C(=O)C=C)C2)c(NC)c1C(N)=O ; C=CC(=O)N1C[C@@H](n2nc(C#Cc3cc4c(cc3Cl)ncn4[C@@H]3CC3(F)F)c(C(N)=O)c2NC)C[C@@H]1COC]

3-(2-{6-chloro-3-[(1R)-2,2-difluorocyclopropyl]-1,3-benzodiazol-5-yl}ethynyl)-1-[(3S,5R)-5-(methoxymethyl)-1-(prop-2-enoyl)pyrrolidin-3-yl]-5-(methylamino)pyrazole-4-carboxamide (stereochemistry arbitrarily assigned). MS ESI calculated for C₂₆H₂₆ClF₂N₇O₃ [M+H]⁺, 558.20, found 558.25; ¹H NMR (400 MHz, CDCl₃) δ 8.07 (s, 1H), 7.91 (s, 1H), 7.84 (s, 1H), 7.29 (s, 2H), 6.45 (dd, J=12.4, 7.3 Hz, 2H), 5.73 (dd, J=8.2, 4.4 Hz, 1H), 5.58-5.30 (m, 2H), 4.60 (di, J=9.0 Hz, 1H), 4.08 (dt, J=19.2, 9.2 Hz, 2H), 3.92 (ci, J=13.5 Hz, 1H), 3.50 (dci, J=22.8, 6.7 Hz, 2H), 3.40 (d, J=5.0 Hz, 3H), 3.06 (ci, J=13.2 Hz, 3H), 2.76-2.71 (m, 1H), 2.35 (dd, J=12.7, 7.2 Hz, 2H), 2.04 (s, 1H).

### Example 289: 3-(2-{6-Chloro-3-[(1S)-2,2-difluorocyclopropyl]-1,3-benzodiazol-5-yl}ethynyl)-1-[(3 S,5R)-5-(methoxymethyl)-1-(prop-2-enoyl)pyrrolidin-3-yl]-5-(methylamino)pyrazole-4-carboxamide

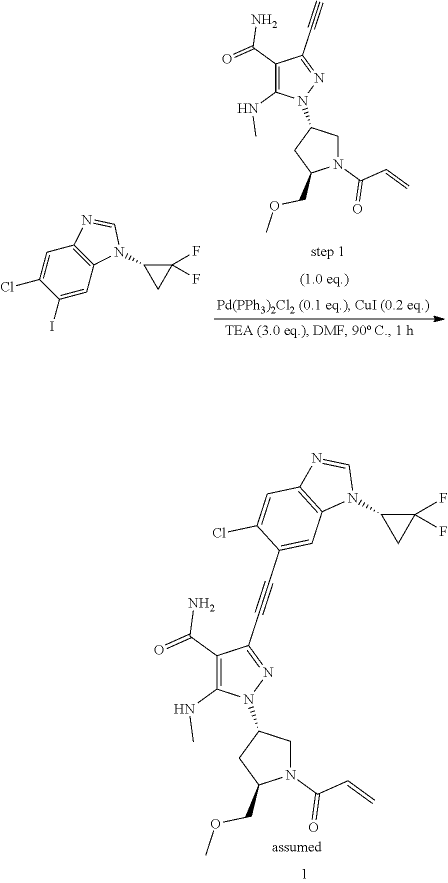 [SMILES: FC1(F)C[C@@H]1n1cnc2cc(Cl)c(I)cc21 ; C#Cc1nn([C@H]2C[C@H](COC)N(C(=O)C=C)C2)c(NC)c1C(N)=O ; C=CC(=O)N1C[C@@H](n2nc(C#Cc3cc4c(cc3Cl)ncn4[C@H]3CC3(F)F)c(C(N)=O)c2NC)C[C@@H]1COC]

3-(2-{6-chloro-3-[(1S)-2,2-difluorocyclopropyl]-1,3-benzodiazol-5-yl}ethynyl)-1-[(3S,5R)-5-(methoxymethyl)-1-(prop-2-enoyl)pyrrolidin-3-yl]-5-(methylamino)pyrazole-4-carboxamide (stereochemistry arbitrarily assigned). MS ESI calculated for C₂₆H₂₆ClF₂N₇O₃ [M+H]⁺, 558.18, found 558.25; ¹H NMR (400 MHz, CDCl₃) δ 8.14 (s, 1H), 7.89 (d, J=25.4 Hz, 2H), 7.21 (s, 1H), 6.57-6.35 (m, 2H), 5.74-5.71 (m, 1H), 5.55-5.51 (m, 1H), 5.44-5.26 (m, 1H), 4.62-4.35 (m, 1H), 4.13-4.05 (m, 2H), 4.01-3.85 (m, 2H), 3.55-3.39 (m, 4H), 3.06 (d, J=13.0 Hz, 3H), 2.76-2.71 (m, 1H), 2.42-2.28 (m, 2H), 2.09-2.02 (m, 1H).

### Example 290: 3-(2-{3-Chloro-6-fluoroimidazo[1,2-a]pyridin-7-yl}ethynyl)-1-[(3S,5R)-5-(methoxymethyl)-1-(prop-2-enoyl)pyrrolidin-3-yl]-5-(methylamino)pyrazole-4-carboxamide

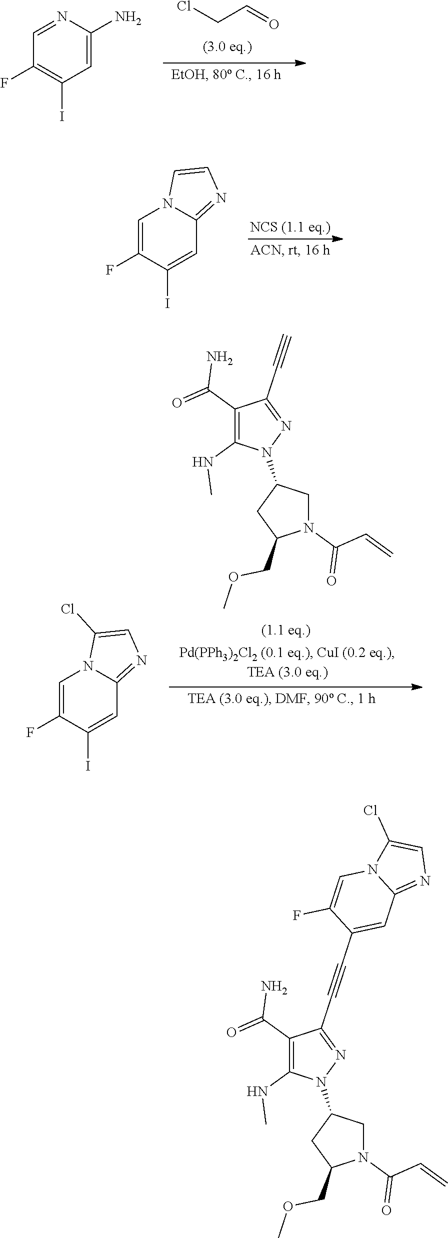

3-(2-{3-chloro-6-fluoroimidazo[1,2-a]pyridin-7-yl}ethynyl)-1-[(3S,5R)-5-(methoxymethyl)-1-(prop-2-enoyl)pyrrolidin-3-yl]-5-(methylamino)pyrazole-4-carboxamide. MS ESI calculated for C₂₃H₂₃ClFN₇O₃ [M+H]⁺, 500.15, found 500.10; ¹H NMR (400 MHz, CDCl₃) δ 8.10 (d, J=4.2 Hz, 1H), 7.90 (d, J=6.3 Hz, 1H), 7.73 (s, 1H), 6.85 (s, 1H), 6.56-6.40 (m, 2H), 5.84-5.73 (m, 1H), 5.60-5.27 (m, 2H), 4.59 (d, J=9.0 Hz, 1H), 4.15-3.99 (m, 2H), 4.10-3.93 (m, 1H), 3.54-3.37 (m, 4H), 3.06 (d, J=14.9 Hz, 3H), 2.71-2.69 (m, 1H), 2.50-2.37 (m, 1H).

### II. Biological Evaluation

### Example 1: FGFR2 Kinase Assay

Small molecule inhibition of FGFR2 kinase activity was evaluated using a fluorescence-based microfluidic mobility shift assay. FGFR2 catalyzes the production of ADP from ATP during phosphoryl transfer to the substrate peptide, FLPeptide30 (5-FAM-KKKKEEIYFFF-CONH2) (Perkin Elmer, 760430). The mobility shift assay electrophoretically separates the fluorescently labeled peptides (substrate and phosphorylated product) following the kinase reaction. Both substrate and product were measured and the ratio of these values used to generate % conversion of substrate to product by the LabChip EZ reader (Perkin Elmer). Wild type FGFR2 (Carna Bioscience, 08-134) at 0.06 nM was prepared with 1.5 M substrate, 10 mM MgCl and 100 μM ATP in a buffer containing 50 nM HEPES, 1 mM EGTA, 0.01% Brij-35, 0.05% BSA, and 2 mM DTT prior to addition of compounds in DMSO and incubation for 80 minutes at room temperature. The reaction was terminated by addition of 0.5 M EDTA. IC₅₀ values were calculated using the inhibition of conversion ratio using Dotmatics Knowledge Solutions Studies curve fitting (Dotmatics, Bishops Stortford, UK, CM23) and are presented in Table 3.

TABLE 3

| Synthetic Chemistry | |
|---|---|
| Example | FGFR2 IC₅₀ |
| 1 | A |
| 2 | A |
| 3 | A |
| 4 | A |
| 5 | A |
| 6 | A |
| 7 | A |
| 8 | A |
| 9 | B |
| 10 | A |
| 11 | A |
| 12 | A |
| 13 | A |
| 14 | A |
| 15 | A |
| 16 | A |
| 17 | A |
| 18 | A |
| 19 | A |
| 20 | A |
| 21 | A |
| 22 | A |
| 23 | A |
| 24 | B |
| 25 | A |
| 26 | B |
| 27 | A |
| 28 | A |
| 29 | A |
| 30 | A |
| 31 | A |
| 32 | A |
| 33 | A |
| 34 | A |
| 35 | A |
| 36 | B |
| 37 | A |
| 38 | A |
| 39 | B |
| 40 | A |
| 41 | B |
| 42 | A |
| 43 | A |
| 44 | A |
| 45 | A |
| 46 | A |
| 47 | A |
| 48 | B |
| 49 | A |
| 50 | A |
| 51 | A |
| 52 | A |
| 53 | B |
| 54 | A |
| 55 | >500 nM |
| 56 | A |
| 57 | >500 nM |
| 58 | >500 nM |
| 59 | A |
| 60 | A |
| 61 | A |
| 62 | A |
| 63 | >500 nM |
| 64 | A |
| 65 | A |
| 66 | A |
| 67 | A |
| 68 | A |
| 69 | A |
| 70 | A |
| 71 | A |
| 72 | A |
| 73 | A |
| 74 | A |
| 75 | A |
| 76 | A |
| 77 | A |
| 78 | A |
| 79 | A |
| 80 | A |
| 81 | A |
| 82 | A |
| 83 | A |
| 84 | A |
| 85 | A |
| 86 | A |
| 87 | A |
| 88 | A |
| 89 | A |
| 90 | A |
| 91 | A |
| 92 | A |
| 93 | A |
| 94 | A |
| 95 | B |
| 96 | A |
| 97 | A |
| 98 | A |
| 99 | A |
| 100 | A |
| 101 | A |
| 102 | A |
| 103 | A |
| 104 | >500 nM |
| 105 | >500 nM |
| 106 | >500 nM |
| 107 | B |
| 108 | A |
| 109 | A |
| 110 | A |
| 111 | A |
| 112 | A |
| 113 | A |
| 114 | A |
| 115 | A |
| 116 | A |
| 117 | A |
| 118 | A |
| 119 | A |
| 120 | A |
| 121 | A |
| 122 | B |
| 123 | A |
| 124 | A |
| 125 | A |
| 126 | A |
| 127 | A |
| 128 | A |
| 129 | B |
| 130 | A |
| 131 | A |
| 132 | A |
| 133 | A |
| 134 | A |
| 135 | A |
| 136 | A |
| 137 | A |
| 138 | A |
| 139 | A |
| 140 | A |
| 141 | A |
| 142 | A |
| 143 | A |
| 144 | A |
| 145 | A |
| 147 | A |
| 148 | A |
| 149 | A |
| 150 | A |
| 151 | A |
| 152 | A |
| 153 | A |
| 154 | A |
| 155 | A |
| 156 | A |
| 157 | A |
| 158 | A |
| 159 | A |
| 161 | A |
| 162 | A |
| 163 | A |
| 164 | A |
| 165 | A |
| 166 | A |
| 167 | A |
| 168 | A |
| 169 | A |
| 170 | A |
| 171 | A |
| 172 | A |
| 173 | A |
| 174 | A |
| 175 | A |
| 176 | A |
| 177 | A |
| 178 | A |
| 179 | A |
| 180 | A |
| 181 | A |
| 182 | A |
| 183 | A |
| 184 | A |
| 185 | A |
| 186 | A |
| 187 | A |
| 188 | A |
| 189 | A |
| 190 | A |
| 191 | A |
| 192 | A |
| 193 | A |
| 194 | A |
| 195 | A |
| 196 | A |
| 197 | A |
| 198 | A |
| 199 | A |
| 200 | A |
| 20 | A |
| 202 | A |
| 203 | A |
| 204 | B |
| 205 | A |
| 206 | A |
| 207 | A |
| 208 | A |
| 209 | A |
| 210 | A |
| 211 | A |
| 212 | A |
| 213 | A |
| 214 | A |
| 215 | A |
| 216 | A |
| 217 | >500 nM |
| 218 | A |
| 219 | A |
| 220 | A |
| 221 | A |
| 222 | A |
| 223 | A |
| 224 | A |
| 225 | A |
| 226 | A |
| 227 | A |
| 228 | A |
| 230 | A |
| 231 | A |
| 232 | A |
| 233 | A |
| 234 | A |
| 235 | A |
| 236 | A |
| 237 | A |
| 238 | A |
| 239 | A |
| 240 | A |
| 241 | A |
| 242 | A |
| 243 | A |
| 244 | A |
| 245 | A |
| 246 | A |
| 247 | A |
| 248 | A |
| 249 | A |
| 250 | A |
| 251 | A |
| 252 | A |
| 253 | A |
| 254 | A |
| 256 | A |
| 257 | A |
| 258 | A |
| 259 | A |
| 260 | A |
| 261 | A |
| 262 | A |
| 263 | A |
| 264 | A |
| 265 | A |
| 266 | A |
| 267 | A |
| 268 | A |
| 269 | A |
| 270 | A |
| 271 | A |
| 272 | A |
| 273 | A |
| 274 | A |
| 275 | A |
| 276 | A |
| 277 | A |
| 278 | A |
| 279 | A |
| 280 | A |
| 281 | A |
| 282 | A |
| 283 | A |
| 284 | B |
| 285 | A |
| 287 | A |
| 288 | A |
| 289 | >500 nM |
| 290 | A |

Note:
Biochemical assay IC₅₀ data are designated within the following ranges:
A: ≤ 0.10 μM
B: > 0.10 μM to ≤ 1.0 μM
C: > 1.0 μM to ≤ 10 μM
D: > 10 μM to 30 μM

### III. Preparation of Pharmaceutical Dosage Forms

### Example 1: Oral Capsule

The active ingredient is a compound of Table 1, or a pharmaceutically acceptable salt or solvate thereof. A capsule for oral administration is prepared by mixing 1-1000 mg of active ingredient with starch or other suitable powder blend. The mixture is incorporated into an oral dosage unit such as a hard gelatin capsule, which is suitable for oral administration.

### Example 2: Solution for Injection

The active ingredient is a compound of Table 1, or a pharmaceutically acceptable salt thereof, and is formulated as a solution in sesame oil at a concentration of 50 mg-eq/mL.

The examples and embodiments described herein are for illustrative purposes only and various modifications or changes suggested to persons skilled in the art are to be included within the spirit and purview of this application and scope of the appended claims.

1. A method of treating cancer in a patient in need thereof, wherein the method comprises administering to the patient a compound, or pharmaceutically acceptable salt or solvate thereof, having the structure of Formula (I): 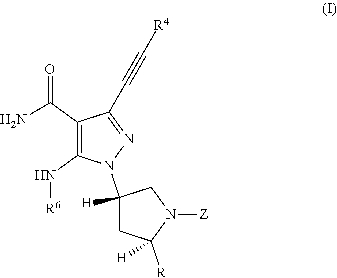
wherein,
Z is selected from a group having the structure: 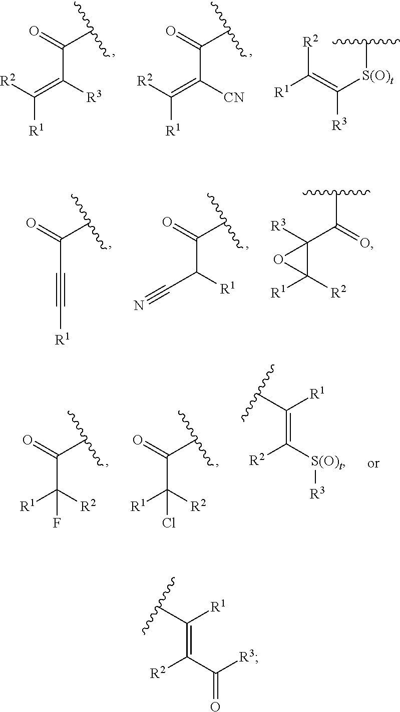
t is 1 or 2;
R¹, R², and R³ are each independently selected from hydrogen, fluoro, optionally substituted C1-C4 alkyl, or optional substituted heterocyclylalkyl;
R⁴ is an optionally substituted nitrogen-containing 9 or 10-atom heteroaryl;
R is selected from hydrogen, optionally substituted C1-C6 alkyl, optionally substituted C3-C7 carbocyclyl, optionally substituted C3-C7 carbocyclylalkyl, optionally substituted C3-C7 heterocyclyl, optionally substituted C3-C7 heterocyclylalkyl, optionally substituted C2-C7 alkenyl, —CO₂R⁵, —CONHR⁵, or —CON(R⁵)₂;
each R⁵ is independently selected from optionally substituted C1-C6 alkyl, optionally substituted C3-C7 carbocyclyl, optionally substituted C3-C7 carbocyclylalkyl, optionally substituted C3-C7 heterocyclyl, or optionally substituted C3-C7 heterocyclylalkyl; and
R⁶ is an optionally substituted alkyl, optionally substituted carbocyclylalkyl, or optionally substituted heterocyclylalkyl.

2. The method of claim 1, or a pharmaceutically acceptable salt or solvate thereof, wherein Z is

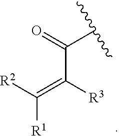

3. The method of claim 1, or pharmaceutically acceptable salt or solvate thereof, wherein R² is hydrogen.

4. The method of claim 1, or pharmaceutically acceptable salt or solvate thereof, wherein R³ is hydrogen or fluoro.

5. (canceled)

6. The method of claim 6, or pharmaceutically acceptable salt or solvate thereof, wherein R¹ is hydrogen.

7. The method of claim 1, or pharmaceutically acceptable salt or solvate thereof, wherein R¹ is optionally substituted C1-C4 alkyl.

8. The method of claim 1, or pharmaceutically acceptable salt or solvate thereof, wherein R¹ is optionally substituted C1-C2 alkyl.

9. The method of claim 1, or pharmaceutically acceptable salt or solvate thereof, wherein R¹ is optionally substituted C1 alkyl.

10. The method of claim 7, or pharmaceutically acceptable salt or solvate thereof, wherein the optionally substituted alkyl is substituted with an optionally substituted amino group.

11. The method of claim 10, or pharmaceutically acceptable salt or solvate thereof, wherein the optionally substituted amino group is a dimethylamino.

12. The method of claim 1, or a pharmaceutically acceptable salt or solvate thereof, wherein R⁴ is selected from optionally substituted benzimidazole, optionally substituted 1H-indazole, optionally substituted 2H-indazole, optionally substituted benzotriazole, optionally substituted benzoxazole, optionally substituted imidazo[4,5-c]pyridine, or optionally substituted imidazo[4,5-b]pyridine.

13. The method of claim 1, or a pharmaceutically acceptable salt or solvate thereof, wherein R⁴ is selected from quinoline, quinoxaline, pyrazolo[1,5-a]pyrimidine, imidazo[1,2-a]pyridine, pyrazolo[1,5-a]pyridine, imidazo[1,2-a]pyrimidine, imidazo[1,2-b]pyridazine, or pyrazolo[1,5-a]pyridine.

14. The method of claim 12, or a pharmaceutically acceptable salt or solvate thereof, wherein R⁴ is an optionally substituted benzimidazole.

15. The method of claim 12, or a pharmaceutically acceptable salt or solvate thereof, wherein R⁴ is an optionally substituted 1H-indazole.

16. The method of claim 12, or a pharmaceutically acceptable salt or solvate thereof, wherein R⁴ is an optionally substituted 2H-indazole.

17. The method of claim 12, or a pharmaceutically acceptable salt or solvate thereof, wherein R⁴ is an optionally substituted benzoxazole, optionally substituted imidazo[4,5-c]pyridine, or optionally substituted imidazo[4,5-b]pyridine.

18. The method of claim 1, or a pharmaceutically acceptable salt or solvate thereof, wherein the R⁴ optionally substituted nitrogen-containing 9 or 10-atom heteroaryl is optionally substituted with alkyl, cycloalkyl, or halogen.

19. The method of claim 14, or a pharmaceutically acceptable salt or solvate thereof, wherein the optionally substituted benzimidazole is optionally substituted with alkyl, cycloalkyl, or halogen.

20. The method of claim 15, or a pharmaceutically acceptable salt or solvate thereof, wherein the optionally substituted 1H-indazole is optionally substituted with alkyl, cycloalkyl, or halogen.

21. The method of claim 16, or a pharmaceutically acceptable salt or solvate thereof, wherein the optionally substituted 2H-indazole is optionally substituted with alkyl, cycloalkyl, or halogen.

22. The method of claim 1, or a pharmaceutically acceptable salt or solvate thereof, wherein R is hydrogen.

23. The method of claim 1, or a pharmaceutically acceptable salt or solvate thereof, wherein R is optionally substituted C1-C6 alkyl.

24. The method of claim 1, or a pharmaceutically acceptable salt or solvate thereof, wherein R is optionally substituted C3-C7 carbocyclyl.

25. The method of claim 1, or a pharmaceutically acceptable salt or solvate thereof, wherein R is optionally substituted C3-C7 carbocyclylalkyl.

26. The method of claim 1, or a pharmaceutically acceptable salt or solvate thereof, wherein R is optionally substituted C3-C7 heterocyclyl, or an optionally substituted C3-C7 heterocyclylalkyl.

27. The method of claim 23, or a pharmaceutically acceptable salt or solvate thereof, wherein the optionally substituted C1-C6 alkyl is a C1-C3 alkyl substituted with a C1-C3 alkoxy.

28. The method of claim 19, or a pharmaceutically acceptable salt or solvate thereof, wherein R⁴ is an optionally substituted benzimidazole further substituted with a cycloalkyl group, and at least one halogen.

29. The method of claim 28, or a pharmaceutically acceptable salt or solvate thereof, wherein R⁴ is an optionally substituted benzimidazole further substituted with a cycloalkyl group, and at least one halogen; R is a —CH₂OCH₃ group; and R⁶ is methyl.

30. (canceled)
